(19) United States
(12) Patent Application Publication
(10) Pub. No.: US 2026/0132119 A1
(43) Pub. Date: May 14, 2026

(54) Alkylphenyl Substituted Compounds, Compositions and Methods of Use

(71) Applicant: Deep Apple Therapeutics, Inc., South San Francisco, CA (US)

(72) Inventors: Craig Stivala, San Mateo, CA (US); Jason R. Zbieg, Montara, CA (US); Liang Zhao, San Mateo, CA (US); Stefan Gahbauer, South San Francisco, CA (US); Roger Benjamin Ruggeri, Waterford, CT (US); Melissa Ashley, Burlingame, CA (US)

(73) Assignee: Deep Apple Therapeutics, Inc., South San Francisco, CA (US)

(21) Appl. No.: 19/381,193

(22) Filed: Nov. 6, 2025

Related U.S. Application Data
(60) Provisional application No. 63/718,119, filed on Nov. 8, 2024.
(60) Provisional application No. 63/725,708, filed on Nov. 27, 2024.
(60) Provisional application No. 63/737,877, filed on Dec. 23, 2024.
(60) Provisional application No. 63/787,567, filed on Apr. 11, 2025.
(60) Provisional application No. 63/787,634, filed on Apr. 11, 2025.
(60) Provisional application No. 63/822,696, filed on Jun. 12, 2025.
(60) Provisional application No. 63/855,793, filed on Aug. 1, 2025.
(60) Provisional application No. 63/878,642, filed on Sep. 9, 2025.
(60) Provisional application No. 63/890,989, filed on Sep. 30, 2025.

(51) Int. Cl. C07D 401/04; A61K 31/192; A61K 31/325; A61K 31/397; A61K 31/40; A61K 31/401; A61K 31/415; A61K 31/4192; A61K 31/423; A61K 31/437; A61K 31/438; A61K 31/4418; A61K 31/4427; A61K 31/4439; A61K 31/444; A61K 31/445; A61K 31/4545; A61K 31/4709; A61K 31/502; A61K 31/506; A61K 31/517; A61K 31/53; A61K 31/5377; A61K 31/55; A61K 38/26; A61K 45/06; A61P 3/04; C07C 233/63; C07C 271/28; C07C 271/38; C07C 275/28; C07D 205/04; C07D 207/08; C07D 207/16; C07D 211/60; C07D 213/61; C07D 223/06; C07D 231/04; C07D 401/12; C07D 401/14; C07D 403/12; C07D 405/12; C07D 405/14; C07D 413/04; C07D 413/14; C07D 417/14; C07D 471/04; C07D 471/10; C07D 491/056; C07D 491/107; C07D 519/00

(52) U.S. Cl. CPC C07D 401/04; A61K 31/192; A61K 31/325; A61K 31/397; A61K 31/40; A61K 31/401; A61K 31/415; A61K 31/4192; A61K 31/423; A61K 31/437; A61K 31/438; A61K 31/4418; A61K 31/4427; A61K 31/4439; A61K 31/444; A61K 31/445; A61K 31/4545; A61K 31/4709; A61K 31/502; A61K 31/506; A61K 31/517; A61K 31/53; A61K 31/5377; A61K 31/55; A61K 38/26; A61K 45/06; A61P 3/04; C07C 233/63; C07C 271/28; C07C 271/38; C07C 275/28; C07D 205/04; C07D 207/08; C07D 207/16; C07D 211/60; C07D 213/61; C07D 223/06; C07D 231/04; C07D 401/12; C07D 401/14; C07D 403/12; C07D 405/12; C07D 405/14; C07D 413/04; C07D 413/14; C07D 417/14; C07D 471/04; C07D 471/10; C07D 491/056; C07D 491/107; C07D 519/00; C07C 2601/08; C07C 2601/14

(57) ABSTRACT

The present disclosure provides compounds that are useful for the treatment of conditions mediated by GIPR. Also provided are pharmaceutical compositions containing such compounds, and methods of treatment using such compounds.

### CROSS REFERENCE TO RELATED APPLICATIONS

The present application claims the benefit of priority to U.S. Application No. 63/718,119, filed Nov. 8, 2024, U.S. Application No. 63/725,708, filed Nov. 27, 2024, U.S. Application No. 63/737,877, filed Dec. 23, 2024, U.S. Application No. 63/787,567, filed Apr. 11, 2025, U.S. Application No. 63/787,634, filed Apr. 11, 2025, U.S. Application No. 63/822,696, filed Jun. 12, 2025, U.S. Application No. 63/855,793, filed Aug. 1, 2025, U.S. Application No. 63/878,642, filed Sep. 9, 2025, and U.S. Application No. 63/890,989, filed Sep. 30, 2025, the entire contents of each of which are incorporated by reference herein for all purposes.

### FIELD OF INVENTION

The present disclosure provides compounds that modulate GIPR and are therefore useful for the treatment of conditions, diseases and/or disorders mediated by GIPR. Also provided are pharmaceutical compositions containing such compounds and processes for preparing such.

### BACKGROUND

People are typically classified as obese when their body mass index (BMI)—a person's weight divided by the square of the person's height—is over 30 kg/m²; the range 25-30 kg/m² is defined as overweight. Obesity is a major cause of disability and is correlated with various diseases and conditions particularly cardiovascular diseases, type 2 diabetes, obstructive sleep apnea, and certain types of cancer and osteoarthritis.

Obesity is a leading preventable cause of death worldwide, with increasing rates in adults and children. In 2022, over 1 billion people were obese worldwide (879 million adults and 159 million children), representing more than a double of adult cases (and four times higher than cases among children) registered in 1990. Obesity is more common in women than in men. The World Health Organization and medical societies, e.g. the American Medical Association classify obesity as a disease.

The GBD [Global Burden of Disease] investigators found an increase in the burden of elevated BMI, with high BMI accounting for 4.0 million deaths in 2015, more than two-thirds of which were caused by cardiovascular disease (CVD), 16 even after accounting for smoking and ill health. Furthermore, a large proportion of both BMI-related deaths (41%) and BMI-related disability-adjusted life-years (34%) were caused by CVD among individuals with obesity. Circulation 2021 143:e984-e1010.

Obesity is also a risk factor for several major cancers, including post-menopausal breast, colorectal, endometrial, kidney, esophageal, pancreatic, liver, and gallbladder cancer. Excess body fat results in an approximately 17% increased risk of cancer-specific mortality. Cancers 2023, 15,485.

Moreover, the trends in obesity prevalence in the United States and around the world highlight the significant impact that obesity will continue to have on CVD incidence, cancer, Type 2 diabetes and other associated disease/conditions.

Recently GLP-1 agents have had considerable success in the treatment of obesity. Yet, many individuals on GLP-1 therapy experience adverse side effects including diarrhea, nausea and vomiting. In addition, GLP-1 agents may not sufficiently reduce fat body mass vs. lean body mass which may be aided through the role of GIPR signaling on fat cells. Further, GLP-1 agents' action on weight loss may diminish with time.

Glucose-dependent insulinotropic polypeptide (GIP) is a single 42-amino acid peptide secreted from K-cells in the small intestine (duodenum and jejunum). Human GIP is derived from the processing of proGIP, 153-amino acid precursor that is encoded by a gene localized to chromosome 17q (Inagaki et al., Endocrinol 1989; 3:1014-1021; Fehmann et al. Endocr Rev. 1995; 16:390-410). GIP was formerly called gastric inhibitory polypeptide.

GIP secretion is induced by food ingestion. GIP has a number of physiological effects in tissues, including promotion of fat storage in adipocytes and promotion of pancreatic islet-cell function and glucose-dependent insulin secretion. GIP and glucagon like polypeptide-I (GLP-1) are known insulinotropic factors (“incretins”). Intact GIP is rapidly degraded by DPPIV to an inactive form. The insulinotropic effect of GIP is lost in type 2 diabetic patients while GLP-1 's incretin effect remains intact (Nauck et al. J. Cline. Invest. 1993; 91:301-307).

The GIP receptor (GIPR) is a member of the secretin-glucagon family of class B G-protein coupled receptors (GPCRs) having an extracellular N-terminus, seven transmembrane domains and an intracellular C-terminus. The N-terminal extracellular domains of this family of receptors are usually glycosylated and form the recognition and binding domain of the receptor. GIPR is highly expressed in a number of tissues, including the pancreas, gut, adipose tissue, heart, pituitary, adrenal cortex, and brain (Usdin et al., Endocrinology. 1993, 133:2861-2870). Human GIPR comprises 466 amino acids and is encoded by a gene located on chromosome 19q13.3 (Gremlich et al., Diabetes. 1995; 44:1202-8; Volz et al., FEBS Lett. 1995, 373:23-29). Studies have suggested that alternative mRNA splicing results in the production of GIP receptor variants of differing lengths in human, rat and mouse.

GIPR knockout mice (Gipr_1_) are resistant to high fat diet-induced weight gain and have improved insulin sensitivity and lipid profiles. (Yamada et al., Diabetes. 2006, 55:S86; Miyawaki et al. Nature Med. 2002, 8:738-742). In addition, a novel small molecule GIPR antagonist SKL-14959 prevents obesity and insulin resistance. (Diabetologia 2008, 51:S373, 44th EASD Annual meeting poster). SKL-14959 polypeptide suppressed body weight gain in Mice (“Gastric inhibitory polypeptide receptor antagonist, SKL-14959, suppressed body weight gain on diet-induced obesity mice” T. Nakamura et al. Obesity Science and Practice January 2018.

There have been additional efforts to discover GIPR agents to aid weight loss. Expert Opinion on Drug Discovery 2023, VOL. 18, NO. 6, 659-669. GIPR antagonist small molecules have been disclosed e.g., WO2009/148004; EP1283058. WO2024/214038 and WO2025/224599 also disclose GIPR antagonist small molecules.

Separately, WO2006/104826 discloses glucagon receptor substituted aryl and heteroaryl derivatives as antagonists for the treatment of diabetes and related conditions.

Accordingly, these links to obesity and insulin resistance imply GIPR modulation is a useful approach for therapeutic intervention. While GLP-1 agents have been beneficial in treating some obese patients, given the prevalence of obesity in modern society and its adverse health effects there is a continuing need for new agents (e.g., GIPR antagonists) that are useful for the treatment of obesity.

### SUMMARY

The present invention relates to compounds of Formula (I), as described herein including stereochemical isomeric forms thereof and pharmaceutically acceptable salts thereof, which are useful as GIPR antagonists and/or treating or preventing GIPR mediated conditions and diseases.

More specifically, in some embodiments, provided herein are compounds of Formula (I) or a pharmaceutically acceptable salt thereof:
1. A compound of Formula (I) or a pharmaceutically acceptable salt thereof:

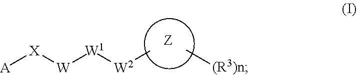

wherein:
A is absent, —(C₀-C₄)alkyl-C(O)OR², OR², or

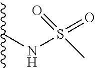 [SMILES: *NS(C)(=O)=O]

wherein the —N(H)S(O)₂—CH₃ is optionally substituted with one to three halogens;
R² is H or (C₁-C₆)alkyl;
X is —W³-L-W⁴—, wherein L is absent, —C(O)—N(H)—, or —N(H)—C(O)—, wherein W³ is bonded to A and W⁴ is bonded to W;
W³ is absent, phenyl, phenyl fused to a 5 to 6 membered heteroaryl, a 5 to 6 membered heteroaryl, or a 5 to 6 membered heteroaryl fused to a 5 to 6 membered heteroaryl, said phenyl fused to a 5 to 6 membered heteroaryl, 5 to 6 membered heteroaryl fused to a 5 to 6 membered heteroaryl, and a 5 to 6 membered heteroaryl having 1 to 3 heteroatoms selected independently from nitrogen, sulfur and oxygen;
wherein W³ is optionally mono-, di-, or tri-substituted independently with hydroxy, halo, oxo, (C₁-C₄)alkyl or (C₁-C₄)alkoxy;
W⁴ is absent, phenyl, 5 to 6 membered heteroaryl, phenyl fused to a (C₃-C₇)cycloalkyl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl, or a phenyl fused to a 5 to 7 membered heteroaryl; wherein said 5 to 6 membered heteroaryl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl and phenyl fused to a 5 to 7 membered heteroaryl have 1 to 4 heteroatoms independently selected from nitrogen, sulfur and oxygen;
wherein when W is absent and W¹ is a ring system, W⁴ optionally shares one atom with W¹ in a spiro linkage;
wherein W⁴ is optionally mono-, di-, or tri-substituted independently with halo, oxo, (C₁-C₄)alkyl or (C₁-C₄)alkoxy;
W is absent, —*C(O)—N(H)—, —*N(H)—C(O)—, or —*N(H)S(O)₂—, wherein the “*” represents the point of attachment of W to X;
W¹ is —CH(Y)—, —C(R¹⁰)(R¹¹)—, (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl;
wherein adjacent ring atoms of each of said (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl are bonded to W and W²;
wherein each of said 4 to 7 membered heterocyclic, 4 to 8 membered bridged heterocyclic, or 4 to 7 membered heterocycloalkenyl have 1 to 3 heteroatoms selected independently from nitrogen, oxygen and sulfur;
wherein R¹⁰ and R¹¹ are each independently H or (C₁-C₄)alkyl; or R¹⁰ and R¹¹ are taken together with the carbon to which they are attached to form a 3 to 6 membered cycloalkyl ring; or R¹⁰ and R¹¹ can be taken together with the carbon to which they are attached to form a (C₅-C₆)cycloalkyl ring fused to a phenyl ring;
wherein each of said (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl is optionally mono- or di-substituted independently with (C₁-C₄)alkyl, halo, (C₁-C₄)alkoxy, or trifluoromethyl, or is optionally mono-substituted with phenyl, or are spiro linked to a cyclopropyl or oxetanyl;
wherein Y is (C₁-C₆)alkyl, phenyl or (C₃-C₇)cycloalkyl) each optionally mono-substituted with phenyl or trifluoromethyl;
W² is —*C(O)—N(H)—, —*C(O)—N(H)—(C₁-C₃)alkyl-, —*NH—C(O)NH—, —*O—C(O)—NH—, —*C(O)—O—, —*S(O)₂—, —*S(O)₂—N(H)—, —*S(O)₂—C(H)₂—, —*N(R⁴)—C(O)NH—, wherein the (C₁-C₃)alkyl is optionally substituted with (C₁-C₃)alkyl, halo, (C₁-C₃)alkoxy or (C₁-C₃)alkoxy(C₁-C₃)alkyl and the “*” represents the point of attachment to W¹;
Z is 5 to 7 membered heterocycloalkyl, phenyl, 5 to 6 membered heteroaryl, C₃-C₆ cycloalkyl, 5-10 membered bridged cycloalkyl, phenyl fused to a 5 to 6 membered heteroaryl, a 5 to 6 membered heteroaryl fused to a 5 to 7 membered cycloalkyl, a 5 to 6 membered heteroaryl fused to a 5 to 7 membered heterocycloalkyl, phenyl fused to a 5 to 7 membered heterocycloalkyl or phenyl fused to a 5 to 7 membered cycloalkyl, wherein said 5 to 7 membered heterocycloalkyl, 5 to 6 membered heteroaryl, phenyl fused to a 5 to 6 membered heteroaryl, 5 to 6 membered heteroaryl fused to a 5 to 7 membered cycloalkyl, 5 to 6 membered heteroaryl fused to a 5 to 7 membered heterocycloalkyl and phenyl fused to a 5 to 7 membered heterocycloalkyl have 1 to 3 heteroatoms selected independently from nitrogen, oxygen and sulfur;
each R³ is independently (C₁-C₆)alkyl, (C₁-C₆)alkoxy, halo, phenyl, mono-, di- or tri-halo(C₁-C₄)alkyl, —SCF₃, —OCF₃, —OCHF₂, (C₃-C₇)cycloalkyl, said (C₃-C₇)cycloalkyl optionally substituted independently with 1 or 2 (C₁-C₄)alkyl, or mono-, di- or tri-halo(C₁-C₄)alkyl;
R⁴ is (C₁-C₄)alkyl or phenyl; and
n is 0, 1, 2, or 3;
with the proviso that W⁴—W—W¹—W²—Z is not

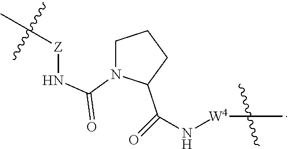

Also provided herein are pharmaceutical compositions comprising a compound of Formula (I) or a pharmaceutically acceptable salt thereof and a pharmaceutically acceptable excipient.

Also provided herein is a pharmaceutical combination composition comprising: a therapeutically effective amount of a composition comprising:
a first compound, said first compound being a compound of Formula I or a pharmaceutically acceptable salt of said compound;
a second compound, said second compound being a GLP-1 inhibitor, a GCGR agonist or a GIPR agonist; and
a pharmaceutically acceptable excipient.

Also provided herein is a pharmaceutical combination composition comprising: a therapeutically effective amount of a composition comprising:
a first compound, said first compound being a compound of Formula I or a pharmaceutically acceptable salt of said compound;
a second compound, said second compound being a GLP-1 inhibitor; and
a third compound, said third compound being a GCGR agonist or a GIPR agonist; and
a pharmaceutically acceptable excipient.

Also provided herein is a kit comprising:
a. a first compound, said first compound being a compound of Formula I, a prodrug thereof, or a pharmaceutically acceptable salt of said compound or of said prodrug and a pharmaceutically acceptable excipient in a first unit dosage form;
b. a second compound, said second compound being a GLP-1 inhibitor, a GCGR agonist or a GIPR agonist and a pharmaceutically acceptable excipient in a second unit dosage form; and
c. means for containing said first and second dosage forms wherein the amounts of first and second compounds result in a therapeutic effect.

Also provided herein is a kit wherein said second compound is a GLP-1 inhibitor.

Further provided herein are methods of modulating GIPR activity; and/or methods of treating a GIPR-mediated disease or disorder in a subject in need thereof, wherein each of these methods independently comprises administering an effective amount of a compound of Formula (I) or a pharmaceutically acceptable salt thereof, or administering an effective amount of a pharmaceutical composition comprising a compound of Formula (I) or a pharmaceutically acceptable salt thereof and a pharmaceutically acceptable excipient.

Also provided is a method of treating a condition chosen from Type 2 diabetes, elevated glucose levels, elevated insulin levels, dyslipidemia, metabolic syndrome (Syndrome X or insulin resistance syndrome), glucosuria, metabolic acidosis, Type 1 diabetes, obesity, conditions exacerbated by obesity, pancreatitis, cirrhosis and Turner's & Cushing's syndrome comprising administering to the subject an effective amount of the compound of Formula (I) and/or a pharmaceutically acceptable salt thereof, or a pharmaceutical composition as described herein.

Also provided herein is a method of treating a disease or condition chosen from Type 2 diabetes, elevated glucose levels, elevated insulin levels, dyslipidemia, metabolic syndrome (Syndrome X or insulin resistance syndrome), glucosuria, metabolic acidosis, Type 1 diabetes and obesity, conditions exacerbated by obesity, pancreatitis, cirrhosis and Turner's & Cushing's syndrome comprising administering to the subject an effective amount of a compound of Formula I or a pharmaceutical composition thereof; and
a second compound, said second compound being a GLP-1 inhibitor or a GCGR agonist.

Also provided herein are compounds of Formula (I) or a pharmaceutically acceptable salt thereof for use as a medicament. In some embodiments the compound is for use in treating disease or disorder mediated by GIPR.

Also provided herein is the use of a compound of Formula (I) or a pharmaceutically acceptable salt thereof in the manufacture of a medicament for treating a GIPR-mediated disease or disorder.

### BRIEF DESCRIPTION OF THE DRAWINGS

FIG. 1 shows the percent of body weight change from baseline in hGIPR mice on a high fat diet (HFD) over twenty-two (22) days. The data shown is mean and the error bars show the standard error of the mean (SEM). *=p<0.0332, ****=p<0.0001 compared with vehicle treated mice.

FIG. 2 shows the cumulative food intake per hGIPR mouse on a high fat diet (HFD) over twenty-two (22) days. The data shown is mean (g) and the error bars show the standard error of the mean (SEM). *=p<0.0032, **=p<0.0021.

### DETAILED DESCRIPTION

Unless otherwise stated, the following terms used in the specification and claims are defined for the purposes of this disclosure and have the following meanings.

“Alkyl” means a saturated, straight or branched hydrocarbon moiety having the specified number of carbon atoms. The term “(C₁-C₆)alkyl” refers to an alkyl moiety containing from 1 to 6 carbon atoms. Exemplary alkyls include, but are not limited to, methyl, ethyl, n-propyl, isopropyl, n-butyl, isobutyl, s-butyl, t-butyl, pentyl, and hexyl. and the like.

When the term “alkyl” is used in combination with other substituent groups, such as “halo(C₁-C₄)alkyl”, “aryl(C₁-C₄)alkyl”, or “(C₁-C₄)alkoxy(C₁-C₄)alkyl”, the term “alkyl” is intended to encompass a divalent straight or branched-chain hydrocarbon radical, wherein the point of attachment is through the alkyl moiety. The term “halo(C₁-C₄)alkyl” is intended to mean a radical having one or more halogen atoms, which may be the same or different, at one or more carbon atoms of an alkyl moiety containing from 1 to 4 carbon atoms, which is a straight or branched-chain carbon radical. Examples of “halo(C₁-C₄)alkyl” groups useful in the present invention include, but are not limited to, mono-, di- or tri-halo(C₁-C₄)alkyl-, or, CF₃ (trifluoromethyl), CCI₃ (trichloromethyl), 1,1-difluoroethyl, 2-fluoro-2-methylpropyl, 2,2-difluoropropyl, 2,2,2-trifluoroethyl, and hexafluoroisopropyl. Examples of “aryl(C₁-C₄)alkyl” or “phenyl(C₁-C₄)alkyl” groups useful in the present invention include, but are not limited to, benzyl and phenethyl. Examples of “(C₁-C₄)alkoxy(C₁-C₄)alkyl” groups useful in the present invention include, but are not limited to, methoxymethyl, methoxyethyl, methoxyisopropyl, ethoxymethyl, ethoxyethyl, ethoxyisopropyl, isopropoxymethyl, isopropoxyethyl, isopropoxyisopropyl, t-butoxymethyl, t-butoxy ethyl, and t-butoxyisopropyl.

“Alkenyl” means a saturated, straight or branched hydrocarbon moiety having the specified number of carbon atoms and at least one carbon-carbon double bond. The term “(C₁-C₆)alkenyl” refers to an alkenyl moiety containing from 1 to 6 carbon atoms. Exemplary alkenyls include, but are not limited to, ethenyl, n-propenyl, isopropenyl, n-butenyl, isobutenyl, s-butenyl, t-butenyl, pentenyl, and hexenyl. and the like.

“Alkoxy” means a —OR radical where R is alkyl as defined above, e.g., methoxy, ethoxy, propoxy, or 2-propoxy, n-, iso-, or tert-butoxy, and the like. The term “(C₁-C₄)alkoxy” refers to a straight- or branched-chain hydrocarbon radical having at least 1 and up to 4 carbon atoms attached through an oxygen linking atom.

As used herein, the term “cycloalkyl” refers to a non-aromatic, saturated, cyclic hydrocarbon ring containing the specified number of carbon atoms. For example, the term (C₃-C₈)cycloalkyl” refers to a non-aromatic cyclic hydrocarbon ring having from three to eight ring carbon atoms. Exemplary “(C₃-C₈)cycloalkyl” groups useful in the present invention include cyclopropyl, cyclobutyl, cyclopentyl, cyclohexyl, cycloheptyl, and cyclooctyl.

As used herein, “heterocycloalkyl” represents a group or moiety comprising a nonaromatic, monocyclic radical, which is saturated or partially unsaturated and includes one, two or three heteroatoms selected independently from oxygen, sulfur, and nitrogen. For example, the term “4- to 6-membered heterocycloalkyl” refers to a heterocycloalkyl group containing 4, 5, or 6 ring atoms, which includes for example, one or two heteroatoms selected independently from oxygen, sulfur, and nitrogen. Illustrative examples of 4- to 6-membered heterocycloalkyl groups useful in the present invention include, but are not limited to azetidinyl, oxetanyl, pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, 1,3-dioxanyl, 1,4-dioxanyl, 1,3-oxathiol any 1, 1,3-oxathianyl, 1,3-dithianyl, 1,4-oxathiolanyl, 1,4-oxathianyl, and 1,4-dithianyl. An example of a partially unsaturated heterocycloalkyl is a heterocycloalkenyl group.

As used herein, “amino” refers to primary, secondary and tertiary nitrogen containing groups.

“Halo” means fluoro, chloro, bromo, or iodo; in one embodiment the halo is fluoro or chloro.

As used herein (unless otherwise specified), “aryl” refers to a monocyclic aromatic ring having 5-8 carbon atoms that complies with Huckel's Rule. As used herein, “arylene” refers to a bivalent aryl group. Examples of “aryl” groups are phenyl, naphthyl, indenyl, dihydroindenyl, anthracenyl, phenanthrenyl, and the like.

“Heteroaryl” as used herein (unless otherwise specified) refers to a group or moiety comprising an aromatic monocyclic ring containing 5 to 8 ring atoms. Illustrative examples of monocyclic and bicyclic groups including a heteroaryl group useful in the present invention include, but are not limited to, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, triazinyl, benzofuranyl, isobenzofuryl, 2,3-dihydrobenzofuryl, 1,3-benzodioxolyl, dihydrobenzodioxinyl, benzothienyl, indolizinyl, indolyl, isoindolyl, dihydroindolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, imidazopyridinyl, pyrazolopyridinyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl. Examples of 5-membered “heteroaryl” groups include furanyl, thienyl, pyrrolyl, imidazolyl, pyrazolyl, triazolyl, tetrazolyl, thiazolyl, oxazolyl, isoxazolyl, oxadiazolyl, thiadiazolyl, and isothiazolyl. Examples of 6-membered “heteroaryl” groups include oxo-pyridyl, pyridinyl, pyridazinyl, pyrazinyl, and pyrimidinyl. For the avoidance of doubt, heteroaryl includes those heteroaryl groups that are substituted with an oxo group, where a tautomer retains aromaticity, for example oxo-pyridyl.

“Oxo” means an ═(O) group where the oxygen is bound to any atom and “carbonyl” means a >C(O) or >C═O or C═O group.

In some embodiments, terms ending in “yl” represent a monovalent group. In some embodiments, terms ending in “yl” represent a bivalent group, or a multivalent group. For example, when G is an alkyl group or cycloalkyl group in R-G, G is monovalent. When G is an alkyl group or cycloalkyl group in R-G-R*, G is bivalent. When G is an alkyl group or cycloalkyl group in R-G(Y)—R*, G is multivalent.

“Mammal” as used herein means domesticated animals (such as dogs, cats, and horses), and humans. In one embodiment, mammal is a human, male or female.

It is to be understood that if a carbocyclic or heterocyclic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3-, or 4-pyridyl, the term “thienyl” means 2-, or 3-thienyl, and so forth.

The term “salt” or “pharmaceutically acceptable salt” refers to salts derived from a variety of organic and inorganic counter ions well known in the art. In some embodiments, due to the acidic nature of the compounds of this invention the pharmaceutically acceptable salts form with base addition salts, such as salts formed with Na⁺ or K⁺ ions.

Generally pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Generally pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

It is understood that the pharmaceutically acceptable salts are non-toxic. For a review on suitable salts, see Handbook of Pharmaceutical Salts. Properties, Selection, and Use by Stahl and Wermuth (Wiley-VCH, 2002). Additional information on suitable pharmaceutically acceptable salts can be found in Remington The Science and Practice of Pharmacy, 23rd ed., Elsevier Science, 2020, which is incorporated herein by reference.

“Optional” or “optionally” means that the subsequently described event or circumstance may but need not occur, and that the description includes instances where the event or circumstance occurs and instances in which it does not. For example, “heterocycloalkyl group optionally substituted with an alkyl group” means that the alkyl may but need not be present, and the description includes situations where the heterocycloalkyl group is substituted with an alkyl group and situations where the heterocycloalkyl group is not substituted with alkyl.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” (includes pharmaceutically acceptable carriers, stabilizers and the like) as described herein. used herein means a

“Treating” or “treatment” of a disease includes:
(1) preventing the disease, e.g., causing the clinical symptoms of the disease not to develop in a mammal that may be exposed to or predisposed to the disease but does not yet experience or display symptoms of the disease;
(2) inhibiting the disease, e.g., arresting or reducing the development or extent of the disease or its clinical symptoms; or
(3) relieving the disease, e.g., causing regression of the disease or its clinical symptoms.
   Thus, prevent, preventing, and the like can refer to the prevention of the disease or condition in the patient. For example, if an individual at risk of contracting a condition/disease is treated with the methods of the present disclosure and does not later contract the condition/disease, then the disease has been prevented, at least over a period of time, in that individual.

A “therapeutically effective amount” means the amount of a compound of Formula (I) (or any of the embodiments thereof described herein), or combination thereof, that when administered to a mammal for treating a disease, is sufficient to treat the disease. The “therapeutically effective amount” will vary depending on the compound, the disease and its severity and the age, weight, etc., of the mammal to be treated.

The compounds described herein may in some cases exist as diastereomers, enantiomers, or other stereoisomeric forms. A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another. or “diastereomers” are stereoisomers that have at least two asymmetric atoms, but which are not mirror-images of each other. Thus, the compounds of the present invention can exist in the form of various stereoisomers, R and S isomers, depending upon the presence of asymmetric carbon atoms. As used herein, they may be referred to as the “R configuration’ or “S configuration” or the like. All chiral, diastereomeric, racemic forms, as individual forms and mixtures thereof, are within the scope of this disclosure, unless the specific stereochemistry or isomeric form is specifically indicated. Compounds of the present disclosure containing an asymmetrically substituted atom may be isolated in optically active, optically enriched, optically pure, or racemic forms. It is well known in the art how to prepare optically active forms, such as by resolution of materials. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis. Those skilled in the art are familiar with methods for determining absolute stereochemistry e.g., X-Ray powder diffraction. Stereoisomers may also be obtained by stereoselective synthesis into their compounding pure enantiomers by forming a diastereomeric salt with an optically pure chiral base or acid (e.g., 1-phenyl-ethylamine or tartaric acid) and separating the diastereomers by fractional crystallization followed by neutralization to break the salt, thus providing the corresponding pure enantiomers.

Certain compounds of Formula (I) (or any of the embodiments thereof described herein) and/or a pharmaceutically acceptable salt thereof may exist as tautomers and/or geometric isomers. All possible tautomers and cis and trans isomers, as individual forms and mixtures thereof, are within the scope of this disclosure.

Additionally, as used herein the term alkyl includes all the possible isomeric forms of said alkyl group albeit only a few examples are set forth. Furthermore, when the cyclic groups such as heteroaryl, heterocyclyl are substituted, they include all the positional isomers.

The compounds described herein include hydrates and solvates of the compounds or pharmaceutically acceptable salts thereof. The term solvate is used herein to describe a molecular complex comprising the compound of the invention and one or more pharmaceutically acceptable solvent molecules, for example, ethanol. Such solvent molecules are those commonly used in the pharmaceutical art, which are known to be innocuous to the recipient, e.g., water, ethanol, and the like. Other solvents may be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like.

The term hydrate is employed when the solvent is water. Pharmaceutically acceptable solvates include hydrates and other solvates wherein the solvent of crystallization may be isotopically substituted, e.g., D₂O. d-acetone, d-DMSO. The solvates and/or hydrates preferably exist in crystalline form. A classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

The present disclosure also includes prodrugs of compounds of Formula (I) (or any of the embodiments thereof described herein) and/or a pharmaceutically acceptable salt thereof. The term prodrug is intended to represent covalently bonded carriers, which are capable of releasing the active ingredient of Formula (I) (or any of the embodiments thereof described herein) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups, however, regenerate original functional groups in vivo or by routine manipulation. Prodrugs of compounds of Formula (I) (or any of the embodiments thereof described herein) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) (or any of the embodiments thereof described herein) and/or a pharmaceutically acceptable salt thereof are also within the scope of this disclosure.

Also included within the scope of the invention are multi-component complexes (other than salts and solvates) wherein the drug and at least one other component are present in stoichiometric or non-stoichiometric amounts. The compounds of the invention may also exist as complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized. For a review of such complexes, see J Pharm Sci, 64 (8), 1269-1288 by Haleblian (August 1975).

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubilizer. Most commonly used for these purposes are alpha-, beta- and gamma-cyclodextrins.

The present disclosure also includes polymorphic forms (amorphous as well as crystalline). The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (“glass transition”). The term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (“melting point”).

Certain compounds of the present invention or combination agents may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present invention followed by gradual or fast cooling. The presence of polymorphs may be determined by solid probe NMR spectroscopy, IR spectroscopy, differential scanning calorimetry, powder X-ray diffraction or other techniques.

The compounds disclosed herein, in some embodiments, are used in different enriched isotopic forms. Thus, the compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵C, ³⁷Cl, ⁷⁹Br, ⁸¹Br, and ¹²⁵I are all contemplated. All isotopic variations of the compounds of the present invention, whether radioactive or not, are encompassed within the scope of the present invention.

For example, compounds having the present structures except for the replacement of a hydrogen by a deuterium or tritium, or the replacement of a carbon by ¹³C- or ¹⁴C-enriched carbon are within the scope of the present disclosure. In certain embodiments, the compounds disclosed herein have some or all of the ¹H atoms replaced with ²H atoms. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs. Deuterium substituted compounds are synthesized using various methods such as described in: Dean, Dennis C.; Editor. Recent Advances in the Synthesis and Applications of Radiolabeled Compounds for Drug Discovery and Development. [In: Cuff., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32. Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Also included within the scope of the invention are metabolites of compounds of Formula I, that is, compounds formed in vivo upon administration of the drug. Some examples of metabolites in accordance with the invention include
(i) where the compound of Formula I contains a methyl group, an hydroxymethyl derivative thereof (—CH₃—>—CH₂OH):
(ii) where the compound of Formula I contains an alkoxy group, an hydroxy derivative thereof (—OR—>—OH);
(iii) where the compound of Formula I contains a tertiary amino group, a secondary amino derivative thereof (—NRR—>—NHR or —NHR);
(iv) where the compound of Formula I contains a secondary amino group, a primary derivative thereof (—NHR—>—NH₂);
(v) where the compound of Formula I contains a phenyl moiety, a phenol derivative thereof (-Ph->-PhOH); and
(vi) where the compound of Formula I contains an amide group, a carboxylic acid derivative thereof (—CONH₂—>COOH).

The present invention may be understood by reference to the following detailed description of exemplary aspects/embodiments of the invention and the examples included herein.

In some embodiments, disclosed herein is a compound of Formula (A) or a pharmaceutically acceptable salt thereof:

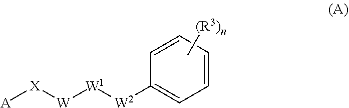

wherein
A is —C(O)OR²;
R² is H or (C₁-C₆)alkyl;
X is —W³—W⁴—;
W³ is absent, phenyl, or a 5 to 6 membered heteroaryl, said heteroaryl having 1 to 2 heteroatoms selected independently from nitrogen, sulfur and oxygen;
wherein W³ is optionally mono-, di-, or tri-substituted independently with halo, (C₁-C₄)alkyl or (C₁-C₄)alkoxy;
W⁴ is absent, phenyl, 5 to 6 membered heteroaryl, phenyl fused to a (C₃-C₇)cycloalkyl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl, or a phenyl fused to a 5 to 7 membered heteroaryl; wherein said 5 to 6 membered heteroaryl, (C₅-C₆) aryl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl and phenyl fused to a 5 to 7 membered heteroaryl have 1 to 4 heteroatoms independently selected from nitrogen, sulfur and oxygen;
wherein W⁴ is optionally mono-, di-, or tri-substituted independently with halo, (C₁-C₄)alkyl or (C₁-C₄)alkoxy;
W is absent, —N(H)—C(O)—, or —N(H)S(O₂)—;
W¹ is —CH(Y)—, —C(R¹⁰)(R¹¹)—, (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl;
wherein adjacent ring atoms of each of said (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl are bonded to W and W²;
wherein each of said 4 to 7 membered heterocyclic, 4 to 8 membered bridged heterocyclic, or 4 to 7 membered heterocycloalkenyl have 1 to 3 heteroatoms selected independently from nitrogen, oxygen and sulfur;
wherein R¹⁰ and R¹¹ are each independently H or (C₁-C₄)alkyl; or R¹⁰ and R¹¹ are taken together with the carbon to which they are attached to form a 3 to 6 membered cycloalkyl ring; or R¹⁰ and R¹¹ can be taken together with the carbon to which they are attached to form a (C₅-C₆)cycloalkyl ring fused to a phenyl ring;
wherein each of said 3 to 6 membered cycloalkyl ring, (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl is optionally mono- or di-substituted independently with (C₁-C₄)alkyl, halo, or trifluoromethyl, or is optionally mono-substituted with phenyl;
wherein Y is (C₁-C₆)alkyl, phenyl or (C₃-C₇)cycloalkyl) each optionally mono-substituted with phenyl or trifluoromethyl;
W² is —C(O)—N(H)—, —NH—C(O)NH—, —O—C(O)—NH—, —C(O)—O—, —S(O)₂—, —S(O)₂—N(H)— or —S(O)₂—C(H)₂—, —N(R⁴)—C(O)NH—;
R³ is (C₁-C₆)alkyl, or halo, —SCF₃, —OCF₃, (C₃-C₇)cycloalkyl;
R⁴ is (C₁-C₄)alkyl or phenyl; and
n is 0, 1, 2, or 3;
with the proviso that W—W¹—W² is not

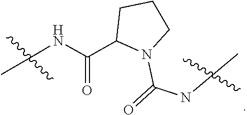 [SMILES: *NC(=O)C1CCCN1C(=O)N*]

In some embodiments, disclosed herein is a compound of Formula (B) or a pharmaceutically acceptable salt thereof:

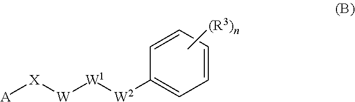

wherein
A is —C(O)OR² or —CH₂—C(O)OR²;
R² is H or (C₁-C₆)alkyl;
X is —W³—W⁴—;
W³ is absent, phenyl, or a 5 to 6 membered heteroaryl, said heteroaryl having 1 to 2 heteroatoms selected independently from nitrogen, sulfur and oxygen;
wherein W³ is optionally mono-, di-, or tri-substituted independently with halo, (C₁-C₄)alkyl or (C₁-C₄)alkoxy;
W⁴ is absent, phenyl, 5 to 6 membered heteroaryl, phenyl fused to a (C₃-C₇)cycloalkyl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl, or a phenyl fused to a 5 to 7 membered heteroaryl; wherein said 5 to 6 membered heteroaryl, (C₅-C₆) aryl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl and phenyl fused to a 5 to 7 membered heteroaryl have 1 to 4 heteroatoms independently selected from nitrogen, sulfur and oxygen;
wherein W⁴ is optionally mono-, di-, or tri-substituted independently with halo, (C₁-C₄)alkyl or (C₁-C₄)alkoxy;
W is absent, —N(H)—C(O)—, or —N(H)S(O₂)—;
W¹ is —CH(Y)—, —C(R¹⁰)(R¹¹)—, (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl;
wherein adjacent ring atoms of each of said (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl are bonded to W and W²;
wherein each of said 4 to 7 membered heterocyclic, 4 to 8 membered bridged heterocyclic, or 4 to 7 membered heterocycloalkenyl have 1 to 3 heteroatoms selected independently from nitrogen, oxygen and sulfur;
wherein R¹⁰ and R¹¹ are each independently H or (C₁-C₄)alkyl; or R¹⁰ and R¹¹ are taken together with the carbon to which they are attached to form a 3 to 6 membered cycloalkyl ring; or R¹⁰ and R¹¹ can be taken together with the carbon to which they are attached to form a (C₅-C₆)cycloalkyl ring fused to a phenyl ring;
wherein each of said 3 to 6 membered cycloalkyl ring, (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl is optionally mono- or di-substituted independently with (C₁-C₄)alkyl, halo, or trifluoromethyl, or is optionally mono-substituted with phenyl;
wherein Y is (C₁-C₆)alkyl, phenyl or (C₃-C₇)cycloalkyl) each optionally mono-substituted with phenyl or trifluoromethyl;
W² is —C(O)—N(H)—, —NH—C(O)NH—, —O—C(O)—NH—, —C(O)—O—, —S(O)₂—, —S(O)₂—N(H)— or —S(O)₂—C(H)₂—, —N(R⁴)—C(O)NH—;
R³ is (C₁-C₆)alkyl, halo, —SCF₃, —OCF₃, (C₃-C₇)cycloalkyl, or (C₃-C₇)cycloalkyl substituted with 1 or 2 (C₁-C₄)alkyl;
R⁴ is (C₁-C₄)alkyl or phenyl; and
n is 0, 1, 2, or 3;
with the proviso that W—W¹—W² is not

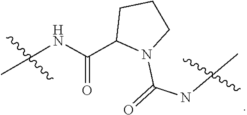 [SMILES: *NC(=O)C1CCCN1C(=O)N*]

In some embodiments, disclosed herein is a compound of Formula (C) or a pharmaceutically acceptable salt thereof:

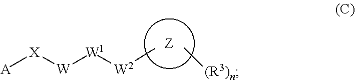

wherein:
A is absent or —(C₁-C₄)alkyl-C(O)OR²;
R² is H or (C₁-C₆)alkyl;
X is —W³—W⁴—;
W³ is absent, phenyl, or a 5 to 6 membered heteroaryl, said heteroaryl having 1 to 2 heteroatoms selected independently from nitrogen, sulfur and oxygen;
wherein W³ is optionally mono-, di-, or tri-substituted independently with halo, oxo, (C₁-C₄)alkyl or (C₁-C₄)alkoxy;
W⁴ is absent, phenyl, 5 to 6 membered heteroaryl, phenyl fused to a (C₃-C₇)cycloalkyl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl, or a phenyl fused to a 5 to 7 membered heteroaryl; wherein said 5 to 6 membered heteroaryl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl and phenyl fused to a 5 to 7 membered heteroaryl have 1 to 4 heteroatoms independently selected from nitrogen, sulfur and oxygen;
wherein W⁴ is optionally mono-, di-, or tri-substituted independently with halo, oxo, (C₁-C₄)alkyl or (C₁-C₄)alkoxy;
W is absent, —N(H)—C(O)—, or —N(H)S(O)₂—;
W¹ is —CH(Y)—, —C(R¹⁰)(R¹¹)—, (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl;
wherein adjacent ring atoms of each of said (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl are bonded to W and W²;
wherein each of said 4 to 7 membered heterocyclic, 4 to 8 membered bridged heterocyclic, or 4 to 7 membered heterocycloalkenyl have 1 to 3 heteroatoms selected independently from nitrogen, oxygen and sulfur;
wherein R¹⁰ and R¹¹ are each independently H or (C₁-C₄)alkyl; or R¹⁰ and R¹¹ are taken together with the carbon to which they are attached to form a 3 to 6 membered cycloalkyl ring; or R¹⁰ and R¹¹ can be taken together with the carbon to which they are attached to form a (C₅-C₆)cycloalkyl ring fused to a phenyl ring;
wherein each of said 3 to 6 membered cycloalkyl ring, (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl is optionally mono- or di-substituted independently with (C₁-C₄)alkyl, halo, or trifluoromethyl, or is optionally mono-substituted with phenyl;
wherein Y is (C₁-C₆)alkyl, phenyl or (C₃-C₇)cycloalkyl) each optionally mono-substituted with phenyl or trifluoromethyl;
W² is —C(O)—N(H)—, —NH—C(O)NH—, —O—C(O)—NH—, —C(O)—O—, —S(O)₂—, —S(O)₂—N(H)— or —S(O)₂—C(H)₂—, —N(R⁴)—C(O)NH—;
Z is phenyl, pyridinyl, 5-10 membered bridged cycloalkyl, or phenyl fused to a 5 to 7 membered heterocycloalkyl, wherein said 5 to 7 membered heterocycloalkyl has 1 to 3 heteroatoms selected independently from nitrogen, oxygen and sulfur; X is —W³—W⁴—;
W³ is absent, phenyl, or a 5 to 6 membered heteroaryl, said heteroaryl having 1 to 2 heteroatoms selected independently from nitrogen, sulfur and oxygen;
wherein W³ is optionally mono-, di-, or tri-substituted independently with halo, oxo, (C₁-C₄)alkyl or (C₁-C₄)alkoxy;
W⁴ is absent, phenyl, 5 to 6 membered heteroaryl, phenyl fused to a (C₃-C₇)cycloalkyl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl, or a phenyl fused to a 5 to 7 membered heteroaryl; wherein said 5 to 6 membered heteroaryl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl and phenyl fused to a 5 to 7 membered heteroaryl have 1 to 4 heteroatoms independently selected from nitrogen, sulfur and oxygen;
wherein W⁴ is optionally mono-, di-, or tri-substituted independently with halo, oxo, (C₁-C₄)alkyl or (C₁-C₄)alkoxy;
W is absent, —N(H)—C(O)—, or —N(H)S(O)₂—;
W¹ is —CH(Y)—, —C(R¹⁰)(R¹¹)—, (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl;
wherein adjacent ring atoms of each of said (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl are bonded to W and W²;
wherein each of said 4 to 7 membered heterocyclic, 4 to 8 membered bridged heterocyclic, or 4 to 7 membered heterocycloalkenyl have 1 to 3 heteroatoms selected independently from nitrogen, oxygen and sulfur;
wherein R¹⁰ and R¹¹ are each independently H or (C₁-C₄)alkyl; or R¹⁰ and R¹¹ are taken together with the carbon to which they are attached to form a 3 to 6 membered cycloalkyl ring; or R¹⁰ and R¹¹ can be taken together with the carbon to which they are attached to form a (C₅-C₆)cycloalkyl ring fused to a phenyl ring;
wherein each of said 3 to 6 membered cycloalkyl ring, (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl is optionally mono- or di-substituted independently with (C₁-C₄)alkyl, halo, or trifluoromethyl, or is optionally mono-substituted with phenyl;
wherein Y is (C₁-C₆)alkyl, phenyl or (C₃-C₇)cycloalkyl) each optionally mono-substituted with phenyl or trifluoromethyl;
W² is —C(O)—N(H)—, —NH—C(O)NH—, —O—C(O)—NH—, —C(O)—O—, —S(O)₂—, —S(O)₂—N(H)— or —S(O)₂—C(H)₂—, —N(R⁴)—C(O)NH—;
Z is phenyl, pyridinyl, 5-10 membered bridged cycloalkyl, or phenyl fused to a 5 to 7 membered heterocycloalkyl, wherein said 5 to 7 membered heterocycloalkyl has 1 to 3 heteroatoms selected independently from nitrogen, oxygen and sulfur;
each R³ is independently (C₁-C₆)alkyl, halo, mono-, di- or tri-halo(C₁-C₄)alkyl, —SCF₃, —OCF₃, or (C₃-C₇)cycloalkyl, said (C₃-C₇)cycloalkyl optionally substituted independently with 1 or 2 (C₁-C₄)alkyl, or mono-, di- or tri-halo(C₁-C₄)alkyl;
R⁴ is (C₁-C₄)alkyl or phenyl; and
n is 0, 1, 2, or 3;
with the proviso that W—W¹—W² is not

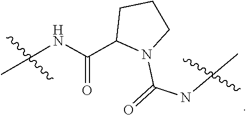 [SMILES: *NC(=O)C1CCCN1C(=O)N*]

In some embodiments, disclosed herein is a compound of Formula (D) or a pharmaceutically acceptable salt thereof:

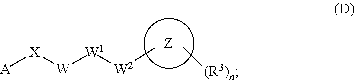

wherein:
A is absent or —(C₀-C₄)alkyl-C(O)OR²;
R² is H or (C₁-C₆)alkyl;
X is —W³—W⁴—, wherein W³ is bonded to A and W⁴ is bonded to W;
W³ is absent, phenyl, phenyl fused to a 5 to 6 membered heteroaryl, or a 5 to 6 membered heteroaryl, said phenyl fused to a 5 to 6 membered heteroaryl and heteroaryl having 1 to 2 heteroatoms selected independently from nitrogen, sulfur and oxygen;
wherein W³ is optionally mono-, di-, or tri-substituted independently with hydroxy, halo, oxo, (C₁-C₄)alkyl or (C₁-C₄)alkoxy;
W⁴ is absent, phenyl, 5 to 6 membered heteroaryl, phenyl fused to a (C₃-C₇)cycloalkyl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl, or a phenyl fused to a 5 to 7 membered heteroaryl; wherein said 5 to 6 membered heteroaryl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl and phenyl fused to a 5 to 7 membered heteroaryl have 1 to 4 heteroatoms independently selected from nitrogen, sulfur and oxygen;
wherein W⁴ is optionally mono-, di-, or tri-substituted independently with halo, oxo, (C₁-C₄)alkyl or (C₁-C₄)alkoxy;
W is absent, —N(H)—C(O)—, or —N(H)S(O)₂—, wherein the X—W—W¹ is X—N(H)—C(O)—W¹ or X—N(H)S(O)₂—W¹;
W¹ is —CH(Y)—, —C(R¹⁰)(R¹¹)—, (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl;
wherein adjacent ring atoms of each of said (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl are bonded to W and W²;
wherein each of said 4 to 7 membered heterocyclic, 4 to 8 membered bridged heterocyclic, or 4 to 7 membered heterocycloalkenyl have 1 to 3 heteroatoms selected independently from nitrogen, oxygen and sulfur;
wherein R¹⁰ and R¹¹ are each independently H or (C₁-C₄)alkyl; or R¹⁰ and R¹¹ are taken together with the carbon to which they are attached to form a 3 to 6 membered cycloalkyl ring; or R¹⁰ and R¹¹ can be taken together with the carbon to which they are attached to form a (C₅-C₆)cycloalkyl ring fused to a phenyl ring;
wherein each of said 3 to 6 membered cycloalkyl ring, (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl is optionally mono- or di-substituted independently with (C₁-C₄)alkyl, halo, (C₁-C₄)alkoxy, or trifluoromethyl, or is optionally mono-substituted with phenyl;
wherein Y is (C₁-C₆)alkyl, phenyl or (C₃-C₇)cycloalkyl) each optionally mono-substituted with phenyl or trifluoromethyl;
W² is —*C(O)—N(H)—, —*C(O)—N(H)—(C₁-C₃)alkyl-, —*NH—C(O)NH—, —*O—C(O)—NH—, —*C(O)—O—, —*S(O)₂—, —*S(O)₂—N(H)— or —*S(O)₂—C(H)₂—, —*N(R⁴)—C(O)NH—, wherein the “*” represents the point of attachment to W¹;
Z is 5 to 7 membered heterocycloalkyl, phenyl, 5 to 6 membered heteroaryl, pyridinyl, C₃-C₆ cycloalkyl, 5-10 membered bridged cycloalkyl, phenyl fused to a 5 to 6 membered heteroaryl, phenyl fused to a 5 to 7 membered heterocycloalkyl or phenyl fused to a 5 to 7 membered cycloalkyl, wherein said 5 to 7 membered heterocycloalkyl, 5 to 6 membered heteroaryl, phenyl fused to a 5 to 6 membered heteroaryl, and 5 to 7 membered heterocycloalkyl have 1 to 3 heteroatoms selected independently from nitrogen, oxygen and sulfur;
each R³ is independently (C₁-C₆)alkyl, halo, phenyl, mono-, di- or tri-halo(C₁-C₄)alkyl, —SCF₃, —OCF₃, —OCHF₂, or (C₃-C₇)cycloalkyl, said (C₃-C₇)cycloalkyl optionally substituted independently with 1 or 2 (C₁-C₄)alkyl, or mono-, di- or tri-halo(C₁-C₄)alkyl;
R⁴ is (C₁-C₄)alkyl or phenyl; and
n is 0, 1, 2, or 3;
with the proviso that W⁴—W—W¹—W²—Z is not

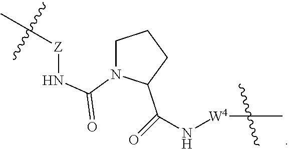

In some embodiments, disclosed herein is a compound of Formula (I) or a pharmaceutically acceptable salt thereof:

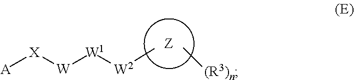

wherein:
A is absent,

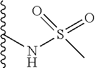 [SMILES: *NS(C)(=O)=O]

optionally substituted with one to three halogens, —(C₀-C₄)alkyl-C(O)OR² or, OR²;
R² is H or (C₁-C₆)alkyl;
X is —W³-L-W⁴—, wherein L is absent, —C(O)—N(H)—, or —N(H)—C(O)—, wherein W³ is bonded to A and W⁴ is bonded to W;
W³ is absent, phenyl, phenyl fused to a 5 to 6 membered heteroaryl, a 5 to 6 membered heteroaryl, or a 5 to 6 membered heteroaryl fused to a 5 to 6 membered heteroaryl, said phenyl fused to a 5 to 6 membered heteroaryl, 5 to 6 membered heteroaryl fused to a 5 to 6 membered heteroaryl, and heteroaryl having 1 to 3 heteroatoms selected independently from nitrogen, sulfur and oxygen;
wherein W³ is optionally mono-, di-, or tri-substituted independently with hydroxy, halo, oxo, (C₁-C₄)alkyl or (C₁-C₄)alkoxy;
W⁴ is absent, phenyl, 5 to 6 membered heteroaryl, phenyl fused to a (C₃-C₇)cycloalkyl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl, or a phenyl fused to a 5 to 7 membered heteroaryl; wherein said 5 to 6 membered heteroaryl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl and phenyl fused to a 5 to 7 membered heteroaryl have 1 to 4 heteroatoms independently selected from nitrogen, sulfur and oxygen;
wherein W⁴ is optionally mono-, di-, or tri-substituted independently with halo, oxo, (C₁-C₄)alkyl or (C₁-C₄)alkoxy;
W is absent, —*C(O)—N(H)—, —*N(H)—C(O)—, or —N(H)S(O)₂—, wherein the X—W—W¹ is X—N(H)—C(O)—W¹ or X—N(H)S(O)₂—W¹, and wherein the “*” represents the point of attachment to W;
W¹ is —CH(Y)—, —C(R¹⁰)(R¹¹)—, (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl;
wherein adjacent ring atoms of each of said (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl are bonded to W and W²;
wherein each of said 4 to 7 membered heterocyclic, 4 to 8 membered bridged heterocyclic, or 4 to 7 membered heterocycloalkenyl have 1 to 3 heteroatoms selected independently from nitrogen, oxygen and sulfur;
wherein R¹⁰ and R¹¹ are each independently H or (C₁-C₄)alkyl; or R¹⁰ and R¹¹ are taken together with the carbon to which they are attached to form a 3 to 6 membered cycloalkyl ring; or R¹⁰ and R¹¹ can be taken together with the carbon to which they are attached to form a (C₅-C₆)cycloalkyl ring fused to a phenyl ring;
wherein each of said 3 to 6 membered cycloalkyl ring, (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl is optionally mono- or di-substituted independently with (C₁-C₄)alkyl, halo, (C₁-C₄)alkoxy, or trifluoromethyl, or is optionally mono-substituted with phenyl, or is substituted with a spiro linked cyclopropyl;
wherein Y is (C₁-C₆)alkyl, phenyl or (C₃-C₇)cycloalkyl) each optionally mono-substituted with phenyl or trifluoromethyl;
W² is —*C(O)—N(H)—, —*C(O)—N(H)—(C₁-C₃)alkyl-, —*NH—C(O)NH—, —*O—C(O)—NH—, —*C(O)—O—, —*S(O)₂—, —*S(O)₂—N(H)— or —*S(O)₂—C(H)₂—, —*N(R⁴)—C(O)NH—, wherein the (C₁-C₃)alkyl is optionally substituted with (C₁-C₃)alkyl, halo, (C₁-C₃)alkoxy or (C₁-C₃)alkoxy(C₁-C₃)alkyl and the “*” represents the point of attachment to W¹;
Z is 5 to 7 membered heterocycloalkyl, phenyl, 5 to 6 membered heteroaryl, C₃-C₆ cycloalkyl, 5-10 membered bridged cycloalkyl, phenyl fused to a 5 to 6 membered heteroaryl, a 5 to 6 membered heteroaryl fused to a 5 to 7 membered cycloalkyl, a 5 to 6 membered heteroaryl fused to a 5 to 7 membered heterocycloalkyl, phenyl fused to a 5 to 7 membered heterocycloalkyl or phenyl fused to a 5 to 7 membered cycloalkyl, wherein said 5 to 7 membered heterocycloalkyl, 5 to 6 membered heteroaryl, phenyl fused to a 5 to 6 membered heteroaryl, 5 to 6 membered heteroaryl fused to a 5 to 7 membered cycloalkyl, 5 to 6 membered heteroaryl fused to a 5 to 7 membered heterocycloalkyl and 5 to 7 membered heterocycloalkyl have 1 to 3 heteroatoms selected independently from nitrogen, oxygen and sulfur;
each R³ is independently (C₁-C₆)alkyl, (C₁-C₆)alkoxy, halo, phenyl, mono-, di- or tri-halo(C₁-C₄)alkyl, —SCF₃, —OCF₃, —OCHF₂, (C₃-C₇)cycloalkyl, said (C₃-C₇)cycloalkyl optionally substituted independently with 1 or 2 (C₁-C₄)alkyl, or mono-, di- or tri-halo(C₁-C₄)alkyl; R⁴ is (C₁-C₄)alkyl or phenyl; and
n is 0, 1, 2, or 3;
with the proviso that W⁴—W—W¹—W²—Z is not

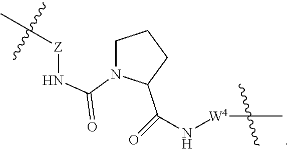

In some embodiments, compound of Formula (I) or a pharmaceutically acceptable salt thereof:

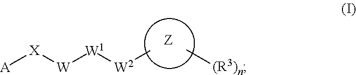

wherein:
A is absent, —(C₀-C₄)alkyl-C(O)OR², OR², or

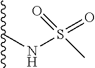 [SMILES: *NS(C)(=O)=O]

wherein the —N(H)S(O)₂—CH₃ is optionally substituted with one to three halogens;
R² is H or (C₁-C₆)alkyl;
X is —W³-L-W⁴—, wherein L is absent, —C(O)—N(H)—, or —N(H)—C(O)—, wherein W³ is bonded to A and W⁴ is bonded to W;
W³ is absent, phenyl, phenyl fused to a 5 to 6 membered heteroaryl, a 5 to 6 membered heteroaryl, or a 5 to 6 membered heteroaryl fused to a 5 to 6 membered heteroaryl, said phenyl fused to a 5 to 6 membered heteroaryl, 5 to 6 membered heteroaryl fused to a 5 to 6 membered heteroaryl, and a 5 to 6 membered heteroaryl having 1 to 3 heteroatoms selected independently from nitrogen, sulfur and oxygen;
wherein W³ is optionally mono-, di-, or tri-substituted independently with hydroxy, halo, oxo, (C₁-C₄)alkyl or (C₁-C₄)alkoxy;
W⁴ is absent, phenyl, 5 to 6 membered heteroaryl, phenyl fused to a (C₃-C₇)cycloalkyl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl, or a phenyl fused to a 5 to 7 membered heteroaryl; wherein said 5 to 6 membered heteroaryl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl and phenyl fused to a 5 to 7 membered heteroaryl have 1 to 4 heteroatoms independently selected from nitrogen, sulfur and oxygen;
wherein when W is absent and W¹ is a ring system, W⁴ optionally shares one atom with W¹ in a spiro linkage;
wherein W⁴ is optionally mono-, di-, or tri-substituted independently with halo, oxo, (C₁-C₄)alkyl or (C₁-C₄)alkoxy;
W is absent, —*C(O)—N(H)—, —*N(H)—C(O)—, or —*N(H)S(O)₂—, wherein the “*” represents the point of attachment of W to X;
W¹ is —CH(Y)—, —C(R¹⁰)(R¹¹)—, (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl;
wherein adjacent ring atoms of each of said (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl are bonded to W and W²;
wherein each of said 4 to 7 membered heterocyclic, 4 to 8 membered bridged heterocyclic, or 4 to 7 membered heterocycloalkenyl have 1 to 3 heteroatoms selected independently from nitrogen, oxygen and sulfur;
wherein R¹⁰ and R¹¹ are each independently H or (C₁-C₄)alkyl; or R¹⁰ and R¹¹ are taken together with the carbon to which they are attached to form a 3 to 6 membered cycloalkyl ring; or R¹⁰ and R¹¹ can be taken together with the carbon to which they are attached to form a (C₅-C₆)cycloalkyl ring fused to a phenyl ring;
wherein each of said (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl is optionally mono- or di-substituted independently with (C₁-C₄)alkyl, halo, (C₁-C₄)alkoxy, or trifluoromethyl, or is optionally mono-substituted with phenyl, or are spiro linked to a cyclopropyl or oxetanyl;
wherein Y is (C₁-C₆)alkyl, phenyl or (C₃-C₇)cycloalkyl) each optionally mono-substituted with phenyl or trifluoromethyl;
W² is —*C(O)—N(H)—, —*C(O)—N(H)—(C₁-C₃)alkyl-, —*NH—C(O)NH—, —*O—C(O)—NH—, —*C(O)—O—, —*S(O)₂—, —*S(O)₂—N(H)—, —*S(O)₂—C(H)₂—, or —*N(R⁴)—C(O)NH—, wherein the (C₁-C₃)alkyl is optionally substituted with (C₁-C₃)alkyl, halo, (C₁-C₃)alkoxy or (C₁-C₃)alkoxy(C₁-C₃)alkyl and the “*” represents the point of attachment to W¹;
Z is 5 to 7 membered heterocycloalkyl, phenyl, 5 to 6 membered heteroaryl, C₃-C₆ cycloalkyl, 5-10 membered bridged cycloalkyl, phenyl fused to a 5 to 6 membered heteroaryl, a 5 to 6 membered heteroaryl fused to a 5 to 7 membered cycloalkyl, a 5 to 6 membered heteroaryl fused to a 5 to 7 membered heterocycloalkyl, phenyl fused to a 5 to 7 membered heterocycloalkyl or phenyl fused to a 5 to 7 membered cycloalkyl, wherein said 5 to 7 membered heterocycloalkyl, 5 to 6 membered heteroaryl, phenyl fused to a 5 to 6 membered heteroaryl, 5 to 6 membered heteroaryl fused to a 5 to 7 membered cycloalkyl, 5 to 6 membered heteroaryl fused to a 5 to 7 membered heterocycloalkyl and phenyl fused to a 5 to 7 membered heterocycloalkyl have 1 to 3 heteroatoms selected independently from nitrogen, oxygen and sulfur;
each R³ is independently (C₁-C₆)alkyl, (C₁-C₆)alkoxy, halo, phenyl, mono-, di- or tri-halo(C₁-C₄)alkyl, —SCF₃, —OCF₃, —OCHF₂, (C₃-C₇)cycloalkyl, said (C₃-C₇)cycloalkyl optionally substituted independently with 1 or 2 (C₁-C₄)alkyl, or mono-, di- or tri-halo(C₁-C₄)alkyl;
R⁴ is (C₁-C₄)alkyl or phenyl; and
n is 0, 1, 2, or 3;
with the proviso that W⁴—W—W¹—W²—Z is not

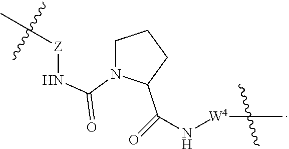

In some embodiments, A is absent, —(C₀-C₄)alkyl-C(O)OR², or —(C₁-C₄)alkyl-C(O)OR².

In some embodiments, A is absent,

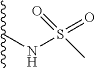 [SMILES: *NS(C)(=O)=O]

optionally substituted with one to three halogens, —(C₀-C₄)alkyl-C(O)OR² or, OR². In some embodiments, A is absent. In some embodiments, A is

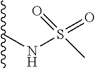 [SMILES: *NS(C)(=O)=O]

optionally substituted with one to three halogens. In some embodiments, A is —(C₀-C₄)alkyl-C(O)OR² or, OR². In some embodiments, A is OR². In some embodiments, A is OH or OCH₃. In some embodiments, A is —C(O)OH. In some embodiments, A is —CH₂—C(O)OH.

In some embodiments, W² is —*C(O)—N(H)—, —*C(O)—N(H)—(C₁-C₃)alkyl-, —*NH—C(O)NH—, —*O—C(O)—NH—, —*C(O)—O—, —*S(O)₂—, —*S(O)₂—N(H)—, —*S(O)₂—C(H)₂—, or —*N(R⁴)—C(O)NH—, wherein the (C₁-C₃)alkyl is optionally substituted with (C₁-C₃)alkyl, halo, (C₁-C₃)alkoxy or (C₁-C₃)alkoxy(C₁-C₃)alkyl and the “*” represents the point of attachment to W¹. In some embodiments, W² is —*C(O)—N(H)—. In some embodiments, W² is —*C(O)—N(H)—(C₁-C₃)alkyl-, wherein the (C₁-C₃)alkyl is optionally substituted with (C₁-C₃)alkyl, halo, (C₁-C₃)alkoxy or (C₁-C₃)alkoxy(C₁-C₃)alkyl. In some embodiments, W² is —*NH—C(O)NH—, —O—C(O)—NH—, —*C(O)—O—.

In some embodiments, A is absent or —(C₀-C₄)alkyl-C(O)OR²; W³ is phenyl; W⁴ is phenyl; and wherein W³ and W⁴ are each independently optionally mono-, di-, or tri-substituted independently with halo, (C₁-C₄)alkyl or (C₁-C₄)alkoxy.

In some embodiments, A is absent or —(C₀-C₄)alkyl-C(O)OR²; W³ is a 5 to 6 membered heteroaryl; and W⁴ is phenyl; and wherein W³ and W⁴ are each independently optionally mono-, di-, or tri-substituted independently with halo, (C₁-C₄)alkyl or (C₁-C₄)alkoxy.

In some embodiments, A is absent or —(C₀-C₄)alkyl-C(O)OR²; W³ is absent; and W⁴ is a 5 to 6 membered heteroaryl fused to a 5 to 6 membered heteroaryl having 1 to 4 heteroatoms selected independently from nitrogen, sulfur and oxygen; and wherein W⁴ is optionally mono-, di-, or tri-substituted independently with halo, oxo, (C₁-C₄)alkyl or (C₁-C₄)alkoxy.

In some embodiments, A is absent or —(C₀-C₄)alkyl-C(O)OR²; W³ is phenyl; W⁴ is phenyl fused to a 5 to 7 membered heteroaryl having 1 to 3 heteroatoms selected independently from nitrogen, sulfur and oxygen; and wherein W³ and W⁴ are each independently optionally mono-, di-, or tri-substituted independently with halo, oxo, (C₁-C₄)alkyl or (C₁-C₄)alkoxy.

In some embodiments, A is absent or —(C₀-C₄)alkyl-C(O)OR²; W³ is phenyl; and W⁴ is a 5 to 6 membered heteroaryl having 1 to 4 heteroatoms selected independently from nitrogen, sulfur and oxygen; and wherein W⁴ is optionally mono-, di-, or tri-substituted independently with halo, oxo, (C₁-C₄)alkyl or (C₁-C₄)alkoxy.

In some embodiments, Z is phenyl. In some embodiments, Z is isopropylphenyl. In some embodiments, Z is 3-fluoro-4-isopropylphenyl. In some embodiments, Z is pyridinyl.

In some embodiments, Z is

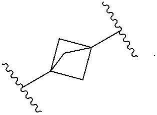 [SMILES: *C12CC(*)(C1)C2]

In some embodiments, Z is

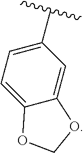 [SMILES: *c1ccc2c(c1)OCO2]

In some embodiments, Z—(R³)ₙ is

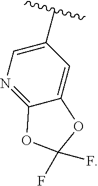 [SMILES: *c1cnc2c(c1)OC(F)(F)O2]

In some embodiments, Z is

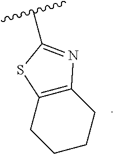 [SMILES: *c1nc2c(s1)CCCC2]

In some embodiments, W is absent, —*C(O)—N(H)—, —*N(H)—C(O)—, or —*N(H)S(O)₂—, wherein the “*” represents the point of attachment of W to X. In some embodiments, W is absent, —C(O)—N(H)—, —N(H)—C(O)—, or —N(H)S(O)₂—, wherein the X—W—W¹ is X—N(H)—C(O)—W¹, X—C(O)—N(H)—W¹, or X—N(H)S(O)₂—W¹. In some embodiments, W is —N(H)—C(O)—, wherein the X—W—W¹ is X—N(H)—C(O)—W¹. In some embodiments, W is —N(H)S(O)₂— wherein the X—W—W¹ is X—N(H)S(O)₂—W¹. In some embodiments, W is absent.

In some embodiments, each R³ is independently (C₁-C₆)alkyl, (C₁-C₆)alkoxy, halo, phenyl, mono-, di- or tri-halo(C₁-C₄)alkyl, —SCF₃, —OCF₃, —OCHF₂, (C₃-C₇)cycloalkyl, said (C₃-C₇)cycloalkyl optionally substituted independently with 1 or 2 (C₁-C₄)alkyl, or mono-, di- or tri-halo(C₁-C₄)alkyl. In some embodiments, R³ is halo and n is 1 or 2. In some embodiments, R³ is (C₁-C₃)alkyl and n is 1. In some embodiments, each R³ is independently (C₁-C₄)alkyl, halo, mono-, di- or tri-halo(C₁-C₄)alkyl and n is 1, 2 or 3.

In some embodiments, W² is —*C(O)—N(H)—, wherein the “*” represents the point of attachment to W¹.

In some embodiments, W¹ is pyrrolidinyl. In some embodiments, W¹ is —C(R¹⁰)(R¹¹)— and R¹⁰ and R¹¹ are taken together with the carbon to which they are attached to form a 3 to 6 membered cycloalkyl ring. In some embodiments, W¹ is —C(R¹⁰)(R¹¹)— and R¹⁰ is (C₁-C₄)alkyl and R¹¹ is H. In some embodiments, W¹ is cyclopentyl. In some embodiments, W¹ is cyclohexyl. In some embodiments, W¹ is cyclohexenyl. In some embodiments, W¹ is azetidinyl. In some embodiments, W¹ is piperidinyl. In some embodiments, W¹ is a 4 to 8 membered bridged heterocyclic. In some embodiments, W¹ is azabicyclo[2.1.1]hexanyl.

In some embodiments, W—W¹—W² is

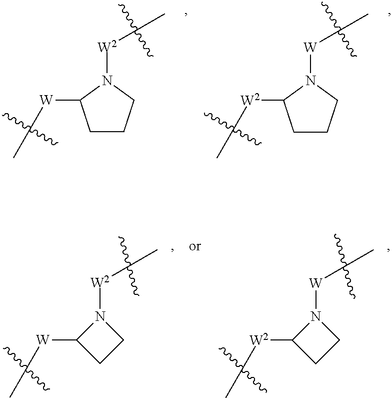

each optionally substituted, for example with one or two methyl groups or with a spiro linked cyclopropyl group. In further embodiment, W is absent.

In some embodiments, W¹ is azetidinyl. In some embodiments, W¹ is azetidinyl and W is absent.

In some embodiments, W—W¹—W² is

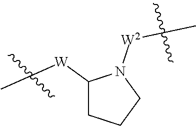

and W is absent. In some embodiments, W—W¹—W² is

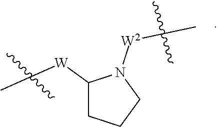

In some embodiments, W¹ is imidazolinyl. In some embodiments, W¹ is pyrazolinyl.

In some embodiments, W³ is unsubstituted. In some embodiments, W³ is mono substituted with fluoro. In some embodiments, W³ is optionally mono-, di-, or tri-substituted independently with hydroxy, halo, oxo, (C₁-C₄)alkyl or (C₁-C₄)alkoxy.

In some embodiments, W⁴ is unsubstituted. In some embodiments, W⁴ is mono-, di-, or tri-substituted independently with halo, oxo, (C₁-C₄)alkyl or (C₁-C₄)alkoxy.

In some embodiments, W is absent and W¹ is a ring system, and W⁴ is linked to W¹ through a bond between one atom in each ring system. In some embodiments, W is absent and W¹ is a ring system, and W⁴ shares one atom with W¹ in a spiro linkage.

In some embodiments, A is —C(O)OH or —CH₂—C(O)OR²; W³ is phenyl; W is —N(H)—C(O)—, wherein the X—W—W¹ is X—N(H)—C(O)—W¹; W² is —*C(O)—N(H)—; R³ is (C₁-C₃)alkyl; n is 1; and W³ is optionally mono-substituted with fluoro or methyl. In some embodiments, W¹ is cyclopentyl. In some embodiments, W¹ is cyclohexyl. In some embodiments, W¹ is cyclohexenyl. In some embodiments, W¹ is azetidinyl. In some embodiments, W¹ is piperidinyl. In some embodiments, W¹ is a 4 to 8 membered bridged heterocyclic. In some embodiments, W¹ is azabicyclo[2.1.1]hexanyl. In some embodiments, W—W¹—W² is

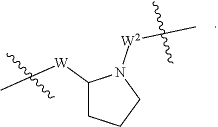

In some embodiments, W¹ is imidazolinyl. In some embodiments, W¹ is pyrazolinyl. In some embodiments, W¹ is cyclopentyl. In some embodiments, W¹ is cyclohexyl. In some embodiments, W¹ is cyclohexenyl. In some embodiments, W¹ is azetidinyl. In some embodiments, W¹ is piperidinyl. In some embodiments, W¹ is a 4 to 8 membered bridged heterocyclic. In some embodiments, W—W¹—W² is azabicyclo[2.1.1]hexanyl. In some embodiments, W—W¹—W² is

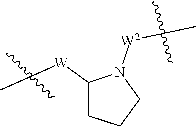

and W is absent. In some embodiments, W—W¹—W² is

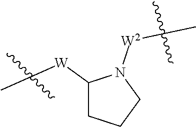

and W is —N(H)—C(O)—, wherein the X—W—W¹ is X—N(H)—C(O)—W¹. In some embodiments, W¹ is imidazolinyl. In some embodiments, W¹ is pyrazolinyl.

In some embodiments, W² is —*NH—C(O)NH—, —*O—C(O)—NH—, —*C(O)—O—, —*S(O)₂—, —*S(O)₂—N(H)— or —*S(O)₂—C(H)₂— or —*N(R⁴)—C(O)NH—, wherein the “*” represents the point of attachment to W¹. In some embodiments, W² is —*NH—C(O)NH—. In some embodiments, W² is —*O—C(O)—NH—. In some embodiments, W² is —*C(O)—O—. In some embodiments, W² is —*S(O)₂—. In some embodiments, W² is —*S(O)₂—N(H)—. In some embodiments, W² is —*S(O)₂—C(H)₂—. In some embodiments, W² is —*N(R⁴)—C(O)NH—. The “*” in the W² group represents the point of attachment to W¹.

In some embodiments, W is —N(H)—C(O)—, wherein the X—W—W¹ is X—N(H)—C(O)—W¹.

In some embodiments, W¹ is (C₃-C₇)cycloalkyl, 4 to 7 membered heterocyclic, 4 to 8 membered bridged heterocyclic, or 4 to 7 membered heterocycloalkenyl having the formula

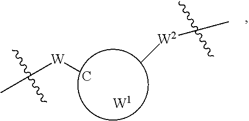

wherein W is bonded to a W¹ ring carbon and said W¹ ring carbon is a stereocenter in an R configuration.

In some embodiments, W¹ is (C₃-C₇)cycloalkyl, 4 to 7 membered heterocyclic, 4 to 8 membered bridged heterocyclic, or 4 to 7 membered heterocycloalkenyl having the formula

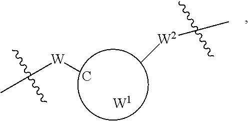

wherein W is bonded to a W¹ ring carbon and said W¹ ring carbon is a stereocenter in an S configuration.

In some embodiments, W¹ is (C₃-C₇)cycloalkyl, 4 to 7 membered heterocyclic, 4 to 8 membered bridged heterocyclic, or 4 to 7 membered heterocycloalkenyl having the formula

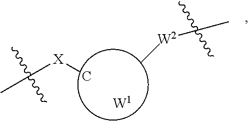

wherein W is absent, X is bonded to a W¹ ring carbon and said W¹ ring carbon is a stereocenter in an R configuration.

In some embodiments, W¹ is (C₃-C₇)cycloalkyl, 4 to 7 membered heterocyclic, 4 to 8 membered bridged heterocyclic, or 4 to 7 membered heterocycloalkenyl having the formula

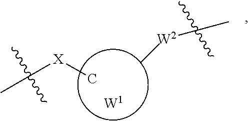

wherein W is absent, X is bonded to a W¹ ring carbon and said W¹ ring carbon is a stereocenter in an S configuration.

In some embodiments, W¹ is (C₃-C₇)cycloalkyl, 4 to 7 membered heterocyclic, 4 to 8 membered bridged heterocyclic, or 4 to 7 membered heterocycloalkenyl having the formula

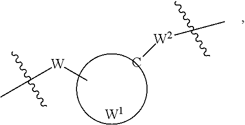

wherein W² is bonded to a W¹ ring carbon and said W¹ ring carbon is a stereocenter in an R configuration.

In some embodiments, W¹ is (C₃-C₇)cycloalkyl, 4 to 7 membered heterocyclic, 4 to 8 membered bridged heterocyclic, or 4 to 7 membered heterocycloalkenyl having the formula

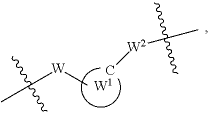

wherein W² is bonded to a W¹ ring carbon and said W¹ ring carbon is a stereocenter in an S configuration.

In some embodiments, W¹ is (C₃-C₇)cycloalkyl, 4 to 7 membered heterocyclic, 4 to 8 membered bridged heterocyclic, or 4 to 7 membered heterocycloalkenyl having the formula

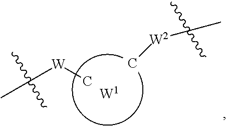

wherein W is bonded to a W¹ ring carbon and said W¹ ring carbon is a stereocenter in an R configuration; and wherein W² is bonded to a W¹ ring carbon and said W¹ ring carbon is a stereocenter in an R configuration.

In some embodiments, W¹ is (C₃-C₇)cycloalkyl, 4 to 7 membered heterocyclic, 4 to 8 membered bridged heterocyclic, or 4 to 7 membered heterocycloalkenyl having the formula

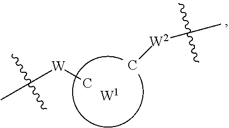

wherein W is bonded to a W¹ ring carbon and said W¹ ring carbon is a stereocenter in an S configuration; and wherein W² is bonded to a W¹ ring carbon and said W¹ ring carbon is a stereocenter in an S configuration.

In some embodiments, W¹ is (C₃-C₇)cycloalkyl, 4 to 7 membered heterocyclic, 4 to 8 membered bridged heterocyclic, or 4 to 7 membered heterocycloalkenyl having the formula

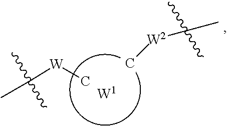

wherein W is absent, X is bonded to a W¹ ring carbon and said W¹ ring carbon is a stereocenter in an R configuration; and wherein W² is bonded to a W¹ ring carbon and said W¹ ring carbon is a stereocenter in an R configuration.

In some embodiments, W¹ is (C₃-C₇)cycloalkyl, 4 to 7 membered heterocyclic, 4 to 8 membered bridged heterocyclic, or 4 to 7 membered heterocycloalkenyl having the formula

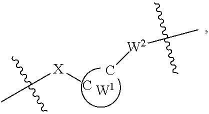

wherein W is absent, X is bonded to a W¹ ring carbon and said W¹ ring carbon is a stereocenter in an S configuration; and wherein W² is bonded to a W¹ ring carbon and said W¹ ring carbon is a stereocenter in an S configuration.

In some embodiments, W¹ is —CH(Y)— and the carbon bonded to (Y) is a stereocenter in an S configuration.

In some embodiments, W¹ is —CH(Y)— and the carbon bonded to (Y) is a stereocenter in an R configuration.

In some embodiments, W—W¹—W² is

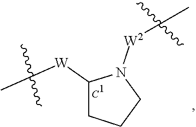

W is absent, and the C¹ carbon is a stereocenter in an R configuration.

In some embodiments, W—W¹—W² is

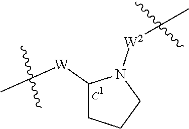

and the C¹ carbon is a stereocenter in an S configuration.

In some embodiments, when an R³ carbon that is bonded to the phenyl ring is a stereocenter the R³ stereocenter carbon is in an R configuration.

In some embodiments, when an R³ carbon that is bonded to the phenyl ring is a stereocenter the R³ stereocenter carbon is in an S configuration.

In some embodiments, A is (C₀-C₄)alkyl-C(O)OR²; R² is H or (C₁-C₆)alkyl; W³ is phenyl optionally mono- or di-substituted independently with halo, (C₁-C₄)alkyl or (C₁-C₄)alkoxy; W⁴ is pyridinyl optionally mono- or di-substituted independently with halo, (C₁-C₄)alkyl or (C₁-C₄)alkoxy; W—W¹—W² is

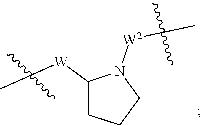

and W is absent; W² is —*C(O)NH—, wherein the “*” represents the point of attachment to W¹; Z is phenyl or pyridinyl; R³ is (C₁-C₆)alkyl, halo, mono-, di- or tri-halo(C₁-C₄)alkyl or (C₃-C₇)cycloalkyl; and n is 1 or 2. In further embodiments, W—W¹—W² is

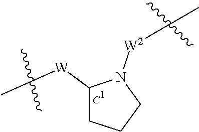

and the C¹ carbon is a stereocenter in an R configuration. In further embodiments, W³ is phenyl optionally mono-substituted independently with halo or (C₁-C₄)alkyl; W⁴ is pyridinyl optionally mono-substituted independently with halo or (C₁-C₄)alkyl; Z is phenyl or pyridinyl; R³ is (C₁-C₆)alkyl, halo or mono-, di- or tri-halo(C₁-C₄)alkyl; and n is 1 or 2.

In some embodiments, A is (C₀-C₄)alkyl-C(O)OR²; R² is H or (C₁-C₆)alkyl; W³ is phenyl optionally mono- or di-substituted independently with halo, (C₁-C₄)alkyl or (C₁-C₄)alkoxy; W⁴ is pyridinyl optionally mono- or di-substituted independently with halo, (C₁-C₄)alkyl or (C₁-C₄)alkoxy; W—W¹—W² is

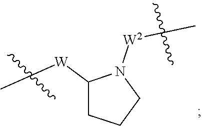

and W is absent; W² is —*C(O)NH—, wherein the “*” represents the point of attachment to W¹; Z is phenyl fused to a 5 to 7 membered heterocycloalkyl; R³ is (C₁-C₆)alkyl, halo, mono-, di- or tri-halo(C₁-C₄)alkyl or (C₃-C₇)cycloalkyl; and n is 1 or 2. In further embodiments, W² is bonded to the Z phenyl.

In some embodiments, A is (C₀-C₄)alkyl-C(O)OR²; R² is H or (C₁-C₆)alkyl; W³ is pyridyl optionally mono- or di-substituted independently with halo, (C₁-C₄)alkyl or (C₁-C₄)alkoxy; W⁴ is phenyl optionally mono- or di-substituted independently with halo, (C₁-C₄)alkyl or (C₁-C₄)alkoxy; W—W¹—W² is

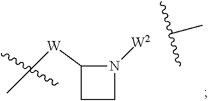

and W is absent; W² is —*C(O)NH—, wherein the “*” represents the point of attachment to W¹; Z is phenyl or pyridinyl; R³ is (C₁-C₆)alkyl, halo, mono-, di- or tri-halo(C₁-C₄)alkyl or (C₃-C₇)cycloalkyl; and n is 1 or 2.

In some embodiments, A is —C(O)OR^{2′} or absent; R² is H; W³ is phenyl, pyridinyl or 1H-[1,2,3]triazolo[4,5-b]pyridin-5-yl optionally mono- or di-substituted independently with (C₁-C₃)alkyl or halo; W⁴ is phenyl or pyridinyl optionally mono- or di-substituted independently with (C₁-C₄)alkyl or halo; W—W¹—W² is

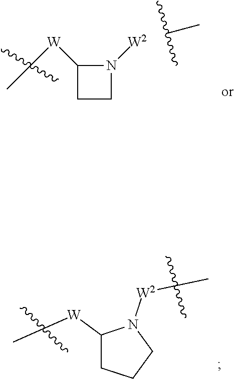

and wherein W¹ is optionally mono- or di-substituted with methyl; and W is absent; W² is —*C(O)NH—, wherein the “*” represents the point of attachment to W¹;
Z is phenyl, pyridinyl or

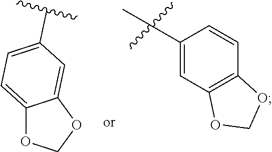 [SMILES: *c1ccc2c(c1)OCO2 ; *c1ccc2c(c1)OCO2]

R³ is (C₁-C₄)alkyl or halo; and n is 1 or 2. In a further embodiment A is —C(O)OR²; R² is H; W³ is phenyl or pyridinyl optionally mono- or di-substituted independently with (C₁-C₃)alkyl, fluoro or chloro; W⁴ is phenyl or pyridinyl optionally mono- or di-substituted independently with fluoro or chloro;
W—W¹—W² is

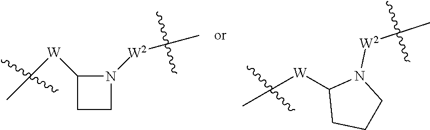

and wherein W¹ is optionally mono- or di-substituted with methyl; and W is absent; W² is —*C(O)NH—, wherein the “*” represents the point of attachment to W¹; Z is phenyl,

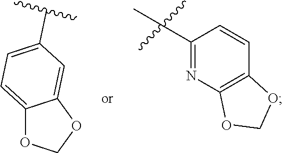 [SMILES: *c1ccc2c(c1)OCO2 ; *c1ccc2c(n1)OCO2]

R³ is (C₁-C₄)alkyl, fluoro or chloro; and
n is 1 or 2. In alternative further embodiment, A is absent; W³ is 1H-[1,2,3]triazolo[4,5-b]pyridin-5-yl; W⁴ is phenyl or pyridinyl optionally mono- or di-substituted independently with fluoro or chloro; W—W¹—W² is

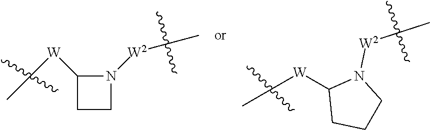

and wherein W¹ is optionally mono- or di-substituted with methyl; and W is absent; W² is —*C(O)NH—, wherein the “*” represents the point of attachment to W¹; Z is phenyl,

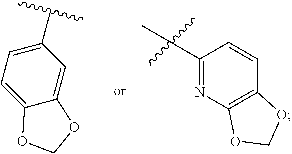 [SMILES: *c1ccc2c(c1)OCO2 ; *c1ccc2c(n1)OCO2]

R³ is (C₁-C₄)alkyl, fluoro or chloro; and n is 1 or 2.

In some embodiments, A is —C(O)OR^{2′} or absent; R² is H; W³ is phenyl, pyridinyl or 1H-[1,2,3]triazolo[4,5-b]pyridin-5-yl optionally mono- or di-substituted independently with (C₁-C₃)alkyl or halo; W⁴ is phenyl or pyridinyl optionally mono- or di-substituted independently with (C₁-C₄)alkyl or halo; W—W¹—W² is

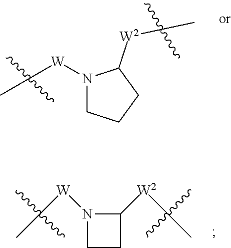

W is absent or —*NHC(O)—, wherein the “*” represents the point of attachment to X; W¹ is optionally mono- or di-substituted with methyl; W² is —*C(O)NH—, wherein the “*” represents the point of attachment to W¹; Z is phenyl, pyridinyl,

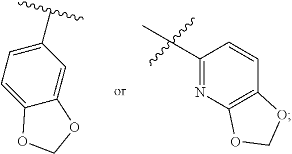 [SMILES: *c1ccc2c(c1)OCO2 ; *c1ccc2c(n1)OCO2]

R³ is (C₁-C₄)alkyl or halo; and n is 1 or 2. In a further embodiment, A is C(O)OR²; R² is H; W³ is phenyl or pyridinyl optionally mono- or di-substituted independently with (C₁-C₃)alkyl, fluoro or chloro; W⁴ is phenyl or pyridinyl optionally mono- or di-substituted independently with fluoro or chloro; W—W¹—W² is

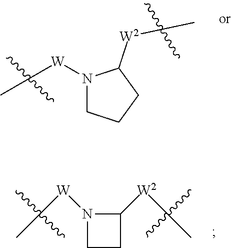

W is absent; W¹ is optionally mono- or di-substituted with methyl; W² is —*C(O)NH—, wherein the “*” represents the point of attachment to W¹; Z is phenyl,

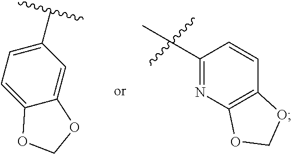 [SMILES: *c1ccc2c(c1)OCO2 ; *c1ccc2c(n1)OCO2]

R³ is (C₁-C₄)alkyl, fluoro or chloro; and n is 1 or 2. In alternative further embodiment, A is C(O)OR²; R² is H; W³ is phenyl or pyridinyl optionally mono- or di-substituted independently with (C₁-C₃)alkyl, fluoro or chloro; W⁴ is phenyl or pyridinyl optionally mono- or di-substituted independently with fluoro or chloro; W—W¹—W² is

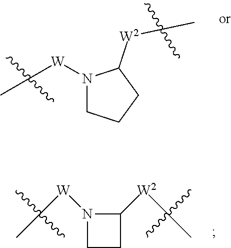

W is —*NHC(O)—, wherein the “*” represents the point of attachment to X; W¹ is optionally mono- or di-substituted with methyl; W² is —*C(O)NH—, wherein the “*” represents the point of attachment to W¹; Z is phenyl,

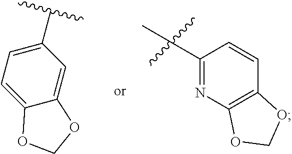 [SMILES: *c1ccc2c(c1)OCO2 ; *c1ccc2c(n1)OCO2]

R³ is (C₁-C₄)alkyl, fluoro or chloro; and n is 1 or 2.

In a further alternative embodiment, A is absent; W³ is 1H-[1,2,3]triazolo[4,5-b]pyridin-5-yl; W⁴ is phenyl or pyridinyl optionally mono- or di-substituted independently with fluoro or chloro; W—W¹—W² is

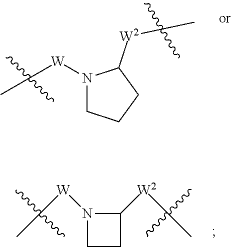

W is absent;
W¹ is optionally mono- or di-substituted with methyl; W² is —*C(O)NH—, wherein the “*” represents the point of attachment to W¹; Z is phenyl,

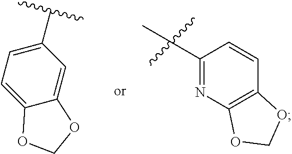 [SMILES: *c1ccc2c(c1)OCO2 ; *c1ccc2c(n1)OCO2]

R³ is (C₁-C₄)alkyl, fluoro or chloro; and n is 1 or 2.

In a further alternative embodiment, A is absent W³ is 1H-[1,2,3]triazolo[4,5-b]pyridin-5-yl; W⁴ is phenyl or pyridinyl optionally mono- or di-substituted independently with fluoro or chloro; W—W¹—W² is

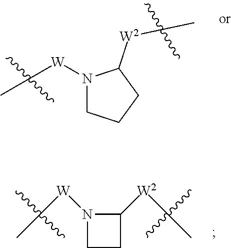

W is —*NHC(O)—, wherein the “*” represents the point of attachment to X; W¹ is optionally mono- or di-substituted with methyl; W² is —*C(O)NH—, wherein the “*” represents the point of attachment to W¹; Z is phenyl,

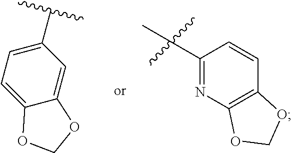 [SMILES: *c1ccc2c(c1)OCO2 ; *c1ccc2c(n1)OCO2]

R³ is (C₁-C₄)alkyl, fluoro or chloro; and n is 1 or 2.

In some embodiments, the compound of Formula (I) is a compound as recited in the Examples and Tables herein, or racemic mixtures thereof, or a pharmaceutically acceptable salt thereof. In some embodiments, the compound of Formula (I) is a compound disclosed herein:

Certain processes for the manufacture of the compounds of this invention are provided as further features of the invention and are illustrated by the examples below. Those skilled in the art will appreciate that other synthetic routes may be used to synthesize the inventive compounds. Although specific starting materials and reagents are depicted in the examples, other starting materials and reagents can be easily substituted to provide a variety of derivatives and/or reaction conditions. In addition, many of the compounds prepared by the methods described below can be further modified in light of this disclosure using conventional chemistry well known to those skilled in the art. In particular, it is noted that the compounds prepared according to these Examples may be modified further to provide new Examples within the scope of this invention. In addition, it will be evident from the detailed descriptions given in the Experimental section that the modes of preparation employed extend further than the procedures described herein. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

The starting materials are generally available from commercial sources such as Merck Sigma-Aldrich Inc. and Enamine Ltd. Aldrich Chemicals (Milwaukee, Wis.) or are readily prepared using methods known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including Supplements (also available via the Beilstein online database).

As an initial note, in the preparation of compounds of the present invention, it is noted that some of the preparation methods useful for the preparation of the compounds described herein may require protection of remote functionality (e.g., primary amine, secondary amine, carboxyl in intermediates). The need for such protection will vary depending on the nature of the remote functionality and the conditions of the preparative methods and can be readily determined by one of ordinary skill in the art. The use of such protection/deprotection methods is also within the ordinary skill in the art. For a general description of protecting groups and their use, see T. W. Greene, Protective Groups in Organic Synthesis, John Wiley & Sons, New York, 1991 and Greene's Protective Groups inorganic Synthesis, John Wiley &Sons, New York 2006.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the Formula I compound.

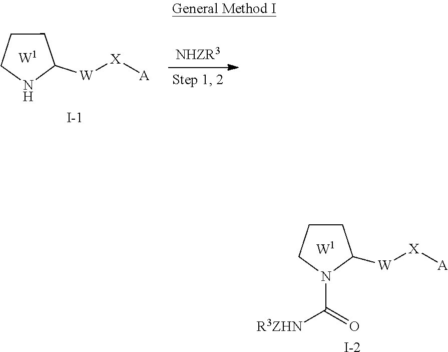

General Method I describes a method for the formation of urea derivatives represented by structural formula I-2. In the scheme, A, W, W¹, X, Z, and R³ are each as defined herein. Although W¹ is shown as a pyrrolidine structure, one skilled in the art will appreciate that this motif may be replaced with other nitrogenous W¹ structures to afford the corresponding products.

In Step 1, an amine represented by NH₂ZR³ is treated with a one-carbon dielectrophile equivalent such as phosgene, triphosgene, or carbonyldiimidazole (CDI), in a polar aprotic solvent such as DCM or DMF, in the presence of an organic base such as N, N-diisopropylethylamine (DIEA) or 4-dimethylaminopyridine (DMAP). The reaction may be conducted at a temperature of −10° C.-60° C., typically at 0° C.-room temperature, and preferably at 0° C. for reactions mediated by phosgene or triphosgene, and at room temperature for those utilizing CDI. After about 1-2 hours at the preferred temperature, the amine I-1 is introduced, and the mixture is stirred for up to 24 hours at a temperature of 0-70° C. Preferably, the reaction is conducted at room temperature for about 2 hours. The product of the reaction is a urea derivative, I-2. In Step 2, for examples where A represents a carboxylic ester functionality such as CH₂CO₂Me or CO₂Me, a saponification may be conducted to produce the corresponding carboxylic acid. This transformation is well known in the art and may be performed with a hydroxide base such as lithium or sodium hydroxide in a polar protic solvent system such as water/THF or water/methanol/THF, within a temperature range of 0° C.-100° C., typically within a range of 0-60° C., and preferably at room temperature. When conducted at room temperature, the reaction is generally complete within 2-4 hours and affords a carboxylic acid. Alternatively, and especially when A represents a tert-butyl ester, the ester may be cleaved under acidic conditions which are well known in the art. An example set of conditions for acid catalyzed hydrolysis of a methyl or ethyl ester include treatment with 6 M HCl at 60° C. for 4-24 hours. An example set of conditions for tert-butyl ester cleavage include treatment with 1-4 M HCl in dioxane, at temperatures ranging from 0° C.-60° C., preferably around room temperature, for 1-18 hours.

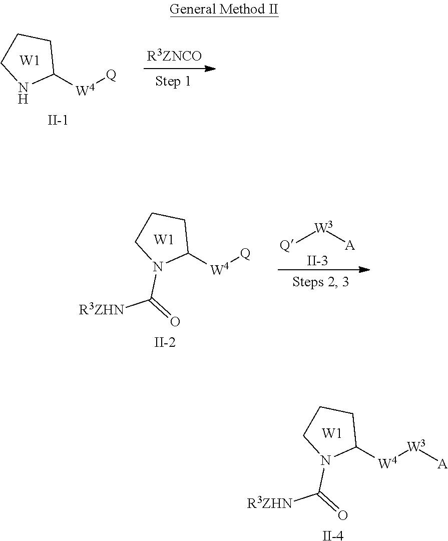

General Method II refers to a synthetic sequence for the preparation of biaryl carboxylic acid derivatives represented by structural formulas II-4. In the scheme, Q and Q′ are I, Br, Cl, B(OH)₂, or B(OR)₂, where R is alkyl, with the proviso that when Q is a halide, Q′ must be a boronate, and vice-versa; the remaining variables are as defined elsewhere herein. Although the accompanying scheme depicts a pyrrolidine structure, one skilled in the art will appreciate that the structure may instead be replaced with other nitrogenous W¹ structures to afford the corresponding products. It will further be appreciated that the sequence order of reactions may be rearranged to-afford the same result. For example, one may conduct the sequence as illustrated, or one may first perform a Suzuki coupling on II-1 or an N-protected variant, followed by subsequent N-deprotection and reaction with R³ZNCO to afford II-4. In Step 1, an amine represented by II-1 is treated with an isocyanate (R³ZNCO), in a polar aprotic solvent such as DCM or 1,4-dioxane, in the presence of an organic base such as triethylamine (TEA) or DIEA, to afford the urea II-2. This reaction typically proceeds within 1-2 hours at room temperature, although certain substrates such as weakly nucleophilic amines may require extended reaction times of up to 24 hours and/or elevated temperatures of up to around 80° C. In Step 2, a Suzuki-Miyaura cross coupling reaction is performed between structures II-2 and II-3 to afford II-4. This type of reaction is well known to those skilled in the art and can be conducted under a variety of conditions. Example conditions include mediation by the catalyst Pd(dppf)Cl₂, in the presence of K₃PO₄, in a solvent system of THF and water or 1,4-dioxane and water, at a temperature of 25° C.-120° C., preferably 60-110° C., and more preferably at 60-90° C. (Suzuki review: Kader, D. A. et al. J. Organomet. Chem. 2025, 1030, 123569). When conducted at the preferred temperature, the Suzuki reaction is typically complete within 1-8 hours and may alternatively be run overnight. In Step 3, for examples of II-4 where A represents carboxylic ester functionality such as CH₂CO₂R or CO₂R, the ester may be cleaved according to methods described previously to produce the corresponding carboxylic acid.

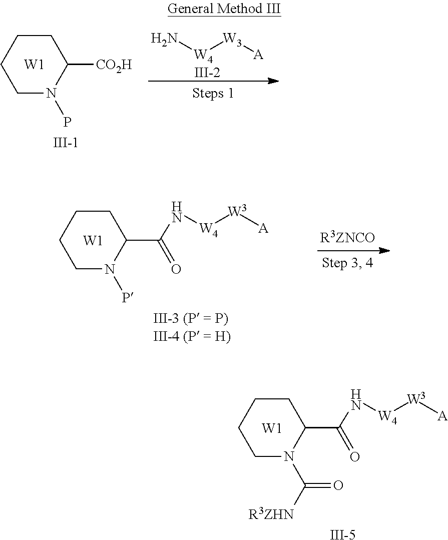

General Method III refers to a synthetic sequence for the preparation of compounds of structural formula III-5. In the scheme, P is a urethane protecting group such as Boc; P′ is H or P, and the remaining variables are as defined elsewhere herein. It will be appreciated by one skilled in the art that the piperidine ring in the scheme may be replaced with other nitrogenous W¹ structures to afford the corresponding products. It will further be appreciated that the sequence of reactions need not necessarily be run in the order depicted. In Step 1, N-protected amino acid derivative III-1 undergoes amidation with amine III-2 (prepared as described herein). There are many ways to conduct amidation reactions with amino acid derivatives, including through treatment with a coupling reagent such as HATU, or with a carbodiimide such as EDCI, or with a uranium salt such as TCFH, or via an acyl chloride or active ester, in apolar aprotic solvent such as DCM, THF, acetonitrile, or DMF, in the presence of an organic base such as TEA, DIEA, NMI, or DBU, with or without racemization suppressing additives such as HOAt or HOBt (Amidation reviews: Valeur, E and Bradley, M. Chem. Soc. Rev. 2009, 38, 606; Humphrey, J. M. and Chamberlin, A. R. Chem. Rev. 1997, 97, 2243). Amidation may be conducted at temperatures ranging from −10° C. to about 80° C., and is typically begun at 0° C. proceeded by warming to room temperature or more for 1-24 hours. The product of the reaction is represented by III-3. In Step 2, Cleavage of the protecting group P′ from III-3 according to well-known procedures affords the amine III-4. For example, if P′ is Boc, the cleavage is commonly achieved with a strong acid such as HCl or TFA in any number of suitable polar aprotic solvents such as DCM, 1,4-dioxane, ethyl acetate, or HFIP at ambient temperature. When P is Cbz, the group may be cleaved by hydrogenation or by strongly acidic conditions. (Greene's Protective Groups in Organic Synthesis, 5th Edition; Wiley, 2014.) In Step 3, the amine III-4 is treated with a commercial isocyanate (R³ZNCO) in a polar aprotic solvent such as DCM or acetonitrile, in the presence of an organic base such as TEA or DIEA, to afford III-5. The typical reaction is begun at 0° C. and is warmed to room temperature for about 2 hours, with sluggish substrates such as sterically hindered or electron deficient amines potentially requiring higher temperatures and/or extended reaction times. In cases where the requisite isocyanate is not commercially available, it may be prepared in situ via the well-known Curtius rearrangement. In this reaction, a carboxylic acid (R³ZCO₂H) undergoes reaction with diphenylphosphoryl azide (DPPA) in an inert solvent such as toluene in the presence of an organic base such as TEA to form an isocyanate in situ. The Curtius reaction can be run between room temperature and 160° C., preferably between 60° C.-100° C., and typically around 80° C., for a period of 1-24 hours, typically around 2 h. Addition of amine III-4 to the in situ-formed isocyanate affords III-5. In Step 4, for examples of III-5 where A represents carboxylic ester functionality such as CH₂CO₂R or CO₂R, the ester may be cleaved according to methods described herein to produce the corresponding carboxylic acid.

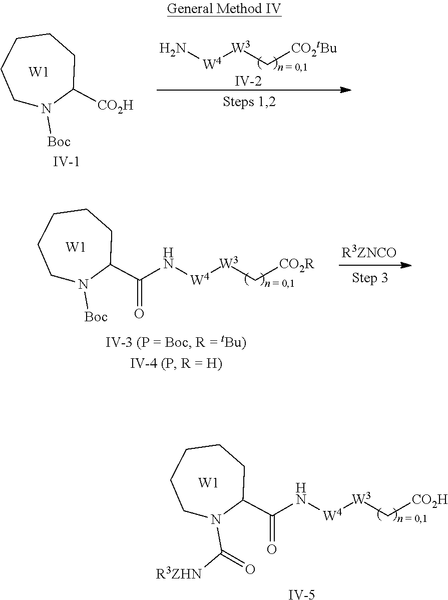

General Method IV describes a synthetic sequence for the preparation of compounds represented by structural formula IV-5. In the scheme, P=H or Boc; R=H or tert-Butyl; and all other variables are as described herein. It will be appreciated by one skilled in the art that the azepine ring depicted in the scheme may be replaced other nitrogenous W¹ structures to afford the corresponding products, and that the sequence of reactions need not necessarily be run in the order depicted. In Step 1, N-Boc derivative IV-1, undergoes an amidation reaction (previously described) with amine IV-2 (prepared as described herein), to afford IV-3. In Step 2, the N-Boc and tert-butyl ester groups are cleaved under strongly acidic conditions as described previously to afford IV-4. In Step 3, IV-4 is treated with an isocyanate, R³ZNCO as described herein to afford the compound IV-5.

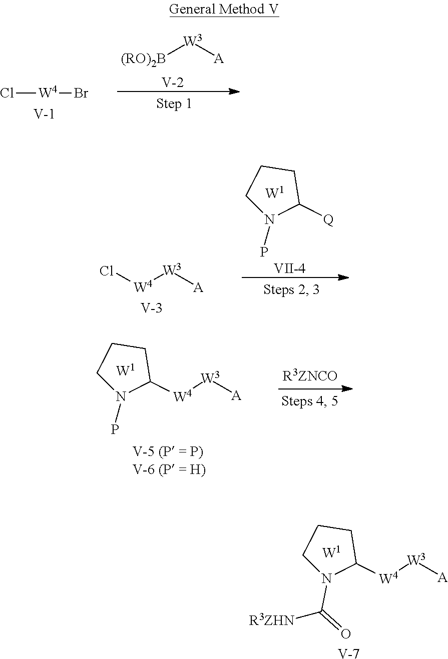

General Method V describes a synthetic sequence for the preparation of compounds represented by structural formula V-7. In the scheme, Br may be Br, Cl, or I; Q is CO₂H, CO₂Pth, or B(OH)₂; R is H or alkyl; P is a urethane protecting group such as Boc; P′ is P or H; and all other variables are as defined herein. It will be appreciated by one skilled in the art that the pyrrolidine ring depicted in the scheme may be replaced with other nitrogenous W¹ structures to afford the corresponding products. Likewise, the Boc group in the scheme may be replaced with other urethane protecting groups and paired with suitable deprotection protocols well known in the art to similar effect. It will be further appreciated that the sequence of transformations need not necessarily be run in the same order as depicted in the scheme. In Step 1, a Suzuki-Miyaura cross coupling reaction (previously described) is performed between two aryl components, represented by a halo species (V-1) and a boronic acid or boronic ester species (V-2), to afford the biaryl species V-3. It will be appreciated by one skilled in the art that the coupling species may be reversed such that V-1 is the boronate species, and V-2 is the halide species. In Step 2, V-3 is subjected to a photocatalytic decarboxylative coupling reaction with an N-protected amino acid derivative represented by V-4 (Q=CO₂H or B(OH)₂). This reaction is accomplished with dual-catalytic system utilizing NiBr₂·Bipy and Ir[dF(CF₃)ppy]₂(dtbpy))PF₆ in the presence of an inorganic base such as cesium carbonate in a polar aprotic solvent, preferably DMF, under blue light irradiation at a wavelength of around 450 nM, preferably at 450 nM. The reaction may be conducted from 0° C.-80° C., preferably room temperature, for a period of up to 24 hours. The product of the reaction is represented by V-5. Alternatively, V-3 may be subjected to a photocatalytic decarboxylative coupling reaction with an N-protected amino acid derivative represented by V-4 (Q=CO₂Pth). This reaction is mediated by the photocatalyst 1,3-Dicyano-2,4,5,6-tetrakis(diphenylamino)-benzene (4-DPAIPN) in a polar aprotic solvent such as acetonitrile in the presence of TFA at a temperature of −10° C.-60° C., preferably 0° C. to room temperature, under blue light irradiation of around 405 nM, preferably at 405 nM, for 1-24 hours, preferrably 12-18 hours, to afford V-5. In Step 3, the urethane protecting group is cleaved using any of a variety of conditions known to those skilled in the art. For example, the N-Boc group can be cleaved under acidic conditions as described herein. The product of Step 3 is an amine, V-6. In Step 4, V-6 is reacted with an isocyanate, R³ZNCO, as described previously to afford V-7. In Step 5, If A includes ester functionality, the ester may be hydrolyzed under acidic conditions or saponified under basic conditions to afford the corresponding carboxylic acid. Each set of conditions is well known to those skilled in the art and was described previously.

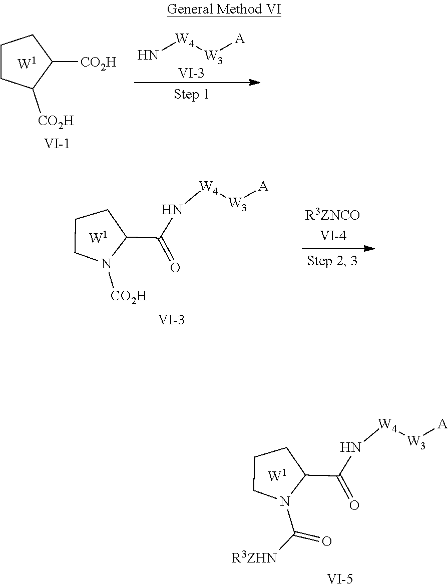

General Method VI refers to a synthetic sequence for the preparation of compounds represented by structural formula VI-5. In the scheme, A, W¹, W³, W⁴, R³ and Z are as defined elsewhere herein. Although the scheme depicts W¹ as a cyclopentyl ring, one skilled in the art will appreciate that this ring may be replaced with alternative W¹ structures to afford the corresponding products. It will be further appreciated that the sequence of transformations need not necessarily be run in the same order as depicted in the scheme. In Step 1, an amidation reaction is conducted between a dicarboxylic acid, represented by VI-1, and an amino component (VI-2). Amidation of carboxylic acids is well known to those skilled in the art and can be accomplished by a variety of methods such as through mediation by acid chlorides, or active esters, or through treatment with propanephosphonic acid cyclic anhydride (T3P). For example, VI-1 may be combined with VI-2 and T3P in an aprotic solvent such as EtOAc, DMF, or pyridine, in the presence of an organic base such as pyridine or triethylamine. The reaction may be run from −10° C. to 100° C., typically from 0° C. to 70° C. Preferably, the reaction mixture is cooled to 0° C. for the addition of the T3P, and is then warmed to room temperature where it is held for up to 24 hours, typically around 2 hours. The product of the reaction is an amide, represented by VI-3. In Step 2, a second T3P coupling is performed between VI-3 and amine component VI-4 to afford the bis-amide structure VI-5. In Step 3, for examples where A possesses ester functionality, the ester group may be hydrolyzed in the presence of strong acid or saponified with a hydroxide base according to methods generally known in the art, and as described herein. For example, if the ester is is tert-butyl, one would select acidic conditions. If R is methyl or ethyl, one may select either acidic or basic conditions for the transformation.

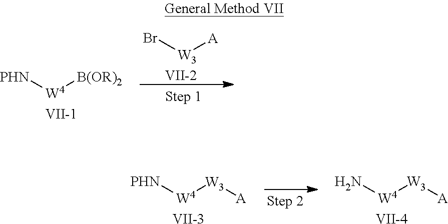

General Method VII describes a synthetic sequence for the preparation of of reaction intermediates represented by structural formula VII-4. In the scheme, P is H or a urethane protecting group such as Boc; R is H or alkyl; Br may be Br, Cl, or I; and W³, W⁴, and A as as described herein. One skilled in the art will recognize that the sequence of steps may be ordered differently than that presented in the scheme. In Step 1, a boronic acid or boronic ester derivative represented by VII-1 is subjected to a Suzuki cross-coupling reaction (described previously) with a halo component, VII-2, to afford the species VII-3 or VII-4. One skilled in the art will recognize that the cross-coupling partners can generally be reversed in this reaction such that VII-1 is the halo component, and VII-2 is the boronate component. In Step 2, if VII-3 possesses a urethane protecting group, the group may be removed using conditions well known in the art. For example, if P is Boc, it may be cleaved under acidic conditions as described herein to afford VII-4.

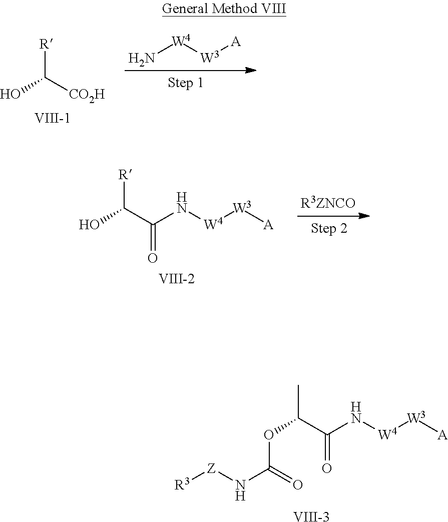

General Method VIII refers to a synthetic sequence for the preparation of compounds represented by structural formula VIII-3. In the Scheme, R′ represents alkyl group configurations defined herein under W¹, and the remaining variables are also as defined herein. It will be appreciated by one skilled in the art that the sequence of transformations need not necessarily be run in the same order as depicted in the scheme. In Step 1, a lactic acid derivative represented by VIII-1 undergoes amidation (described herein) with the amine NH₂W⁴W³A (prepared as described herein) to afford the amide product VIII-2. In Step 2, a solution of VIII-2 and an organic amine such as TEA in a polar aprotic solvent such ac DCM is treated dropwise with an isocyanate (R³ZNCO). The reaction can be run at a temperature of 0° C. to 80° C., typically 0° C.-60° C. Preferred conditions include addition of the isocyanate at 0° C. followed by warming to room temperature for 2-8 hours.

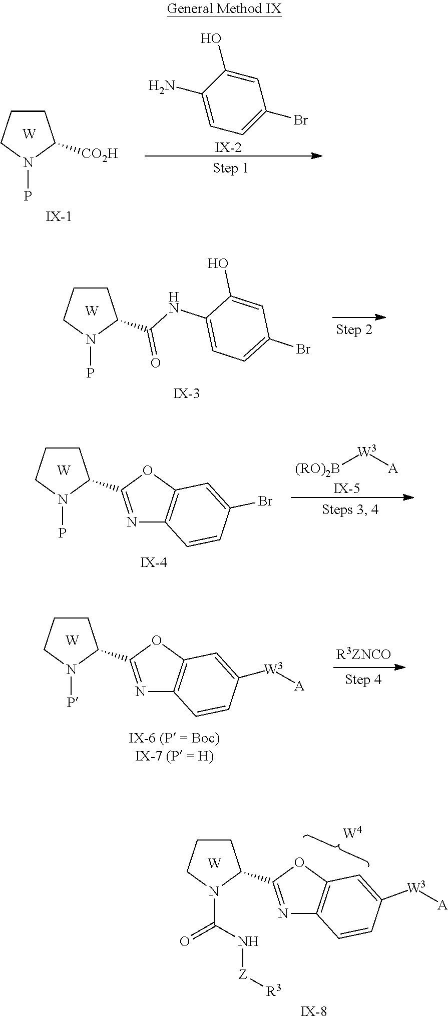

General Method IX refers to a synthetic sequence for the preparation of compounds represented by structural formula IX-8. In the scheme, Br may be Br, Cl, or I; P is a urethane protecting group such as Boc; P′ is H or P; R is H or alkyl; and the remaining variables are as defined elsewhere herein. In Step 1, an amidation reaction (described previously) is conducted between IX-1 and IX-2 to afford IX-3. In step 2, an intramolecular Mitsunobu dehydration is performed on IX-3 to afford IX-4. In this reaction, a mixture of IX-3 and triphenylphosphine in a polar aprotic solvent such as THF is treated with a dialkyl azodicarboxylate such as DIAD or DEAD. This reaction can be run between −10° C. and 80° C. Typically, the DIAD or DEAD is introduced at 0° C., and the mixture is then warmed to room temperature for 2-24 hours, preferably from 2-6 h. In Step 3, a Suzuki-Miyaura cross coupling reaction (previously described) is conducted between IX-4 and IX-5 to afford IX-6. One skilled in the art will recognize that the Suzuki coupling partners can generally be reversed, such that IX-4 may represent the boronate and IX-5 may represent the halide. In Step 4, the urethane protecting group is removed using well-known methods to afford IX-7. For example, if P′ is Boc, it may be removed under acidic conditions as described previously. In Step 4, reaction with a commercial isocyanate or one prepared in situ via the Curtius rearrangement as previously described affords the urea derivative IX-8. For examples where A is a carboxylic ester, the ester may be saponified or hydrolyzed via methods described herein to afford the corresponding carboxylic acid.

### Pharmaceutical Dosages, Compositions and Formulations

The compounds or pharmaceutical compositions described herein that can be used in therapy can be formulated and dosages established in a fashion consistent with good medical practice taking into account the disorder to be treated, the condition of the individual patient, the site of delivery of the compound or pharmaceutical composition, the method of administration and other factors known to practitioners. The compounds or pharmaceutical compositions can be prepared according to the description of preparation described herein.

A therapeutically effective amount can be the amount of a compound or pharmaceutical composition or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment and can be ascertainable by one skilled in the art using known techniques.

One of ordinary skill in the art would understand that the amount, duration, and frequency of administration of a pharmaceutical composition or compound described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

Pharmaceutical compositions or compounds of the present disclosure can be administered to a subject in need thereof in a first administration, and in one or more additional administrations. The one or more additional administrations can be administered to the subject in need thereof minutes, hours, days, weeks, or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week, or more than once per month. The compounds or pharmaceutical compositions can be administered to the subject in need thereof in cycles of 21 days, 14 days, 10 days, 7 days, 4 days, or daily over a period of one to seven days.

In general, the compounds of this disclosure will be administered in a therapeutically effective amount by any of the accepted modes of administration for agents that serve similar utilities. Therapeutically effective amounts of compounds of Formula (I) may range from about 0.01 to about 500 mg/kg patient body weight per day, which can be administered in single or multiple doses. In one embodiment, the dosage level will be about 0.01 to about 250 mg/kg per day, about 0.05 to about 100 mg/kg per day, about 0.1 to about 250 mg/kg per day, about 0.1 to about 50 mg/kg per day or about 0.5 to about 100 mg/kg per day. In addition, the dosage can be about 0.05 to about 0.5, about 0.5 to about 5 or about 5 to about 50 mg/kg per day. For oral administration, the compositions may be provided in the form of tablets containing about 1.0 to about 1000 milligrams of the active ingredient, particularly about 1.0, 5.0, 10, 15, 20, 25, 50, 75, 100, 150, 200, 250, 300, 400, 500, 600, 750, 800, 900, and 1000 milligrams of the active ingredient. The actual amount of the compound of this disclosure, i.e., the active ingredient, will depend upon numerous factors such as the severity of the disease to be treated, the age and relative health of the subject, the potency of the compound being utilized, the route and form of administration, and other factors.

These dosages are based on an average human subject having a weight of about 60 kg to 70 kg. The physician will readily be able to determine doses for subjects whose weight falls outside this range, such as infants and the elderly. Dosage regimens may be adjusted to provide the optimum desired response. For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian Subjects to be treated; each unit containing a predetermined quantity of active compound calculated to produce the desired therapeutic effect in association with the required pharmaceutical carrier. The specification for the dosage unit forms of the invention is dictated by and directly dependent on (a) the unique characteristics of the chemotherapeutic agent and the particular therapeutic or prophylactic effect to be achieved, and (b) the limitations inherent in the art of compounding Such an active compound for the treatment of sensitivity in individuals.

Thus, one of skill in the art would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

In general, compounds of this disclosure will be administered as pharmaceutical compositions by any one of the following routes: oral, systemic (e.g., transdermal, intranasal or by suppository), or parenteral (e.g., intramuscular, intravenous, or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions. The compounds of the invention may also be administered topically, (intra)dermally, or transdermally to the skin or mucosa. Parenteral administration and administered parenterally as used herein includes modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Also provided, in some embodiments, is a pharmaceutical composition comprising the compound of Formula (I) and/or a pharmaceutically acceptable salt thereof and a pharmaceutically acceptable excipient.

A pharmaceutically acceptable excipient includes pharmaceutically acceptable material, composition, or vehicle, such as a liquid or solid filler, diluent, solvent or encapsulating material. Each excipient must be “acceptable” in the sense of being compatible with the other ingredients of the formulation and not injurious to the patient. Some examples of materials which can serve as pharmaceutically acceptable excipients include: (1) monosaccharides, disaccharides, and other carbohydrates including glucose, sucrose; lactose; mannose, trehalose, sorbitol or dextrins and (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide, aluminum hydroxide phosphate, citrate, and other organic acids; (15) alginic acid; (16) pyrogen-free water; (17) isotonic saline; (18) Ringer's solution; (19) ethyl alcohol; (20) antioxidants including ascorbic acid and methionine; preservatives, polypeptides; proteins, such as serum albumin; hydrophilic polymers; amino acids; chelating agents such as EDTA; salt-forming counter-ions such as sodium; metal complexes; and/or non-ionic surfactants or polyethylene glycol and other non-toxic compatible substances employed in pharmaceutical formulations.

Examples of excipients and their use may be found in Remington's Pharmaceutical Sciences, 20th Edition (Lippincott Williams & Wilkins, 2000). The choice of excipient will to a large extent depend on factors such as the particular mode of administration, the effect of the excipient on solubility and stability, and the nature of the dosage form.

Methods for formulation of pharmaceutical compositions can include formulating any of the compounds described herein with one or more inert, pharmaceutically acceptable excipients (including carriers) to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically acceptable additives. This formulation can be an aqueous solution. Alternatively, the compositions described herein can be lyophilized or in powder form for re-constitution with a suitable vehicle, e.g., sterile pyrogen-free water, before use. The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nanoparticles and nanocapsules) or in macroemulsions.

The pharmaceutical compositions and formulations can be sterilized. Sterilization can be accomplished by filtration through sterile filtration.

The pharmaceutical compositions described herein can be formulated for administration as an injection. Non-limiting examples of formulations for injection can include a sterile suspension, solution, or emulsion in oily or aqueous vehicles. Suitable oily vehicles can include, but are not limited to, lipophilic solvents or vehicles such as fatty oils, synthetic fatty acid esters, or liposomes. Aqueous injection suspensions can contain substances which increase the viscosity of the suspension. The suspension can also contain suitable stabilizers. Injections can be formulated for bolus injection or continuous infusion.

For parenteral administration, the compounds can be formulated in a unit dosage injectable form (e.g., solution, suspension, emulsion) in association with a pharmaceutically acceptable parenteral vehicle. Such vehicles can be inherently nontoxic, and non-therapeutic. A vehicle can be water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

The pharmaceutical compositions provided herein may be formulated as immediate or modified release dosage forms, including delayed-, sustained, pulsed-, controlled, targeted-, and programmed-release forms. Thus, in some preferred embodiments, the active ingredient(s) is administered in a pharmaceutical composition which is an immediate release oral dosage form, preferably but not necessarily including an enteric coating. In some preferred embodiments, the active ingredients(s) are administered in a pharmaceutical composition which is an extended-release oral dosage form, preferably but not necessarily including an enteric coating. In further preferred embodiments, the active ingredients are administered in a pharmaceutical composition which contains both an immediate release dose and an extended-release dose or pulsed release dose of the first agent preferably but not necessarily also including an enteric coating. Such dual release dosage forms achieve release of an initial dose of active ingredient, followed late in time by another pulsed release, or by a sustained release dose. Methodologies for preparing such dual release dosage forms are well known in the art.

In some embodiments, the active ingredients are formulated into a controlled release matrix tablet, which contains one or more polymeric matrix materials that promote the sustained, delayed or pulsed release profile. Non-limiting examples of such polymeric matrix materials include cellulosic materials as described above, and carbomers, for example those sold by Lubrizol Corporation under the name Carbopol®, for example Carbopol® 71G NF, Carbopol® 971P NF and Carbopol® 974P NF polymers.

Some preferred examples of extended-release compositions suitable for use in the methods and compositions of the invention include, for example and not limitation, extended-release compositions found in nifedipine formulations such as Adalat CC®, Procardia® XL, Afeditab® CR and Nifedical® XL; and in diltiazem formulations such as Cardizem® CD, Cardizem® LA, Cardizem® SR, Cartia® XT and Dilacor® XR.

### Combinations

Compounds of the present disclosure may be used in methods of treating in combination with one or more other combination agents (e.g., one, two, or three other drugs) that are used in the prevention, treatment, control, amelioration, or reduction of risk of the diseases or conditions for which compounds of the present disclosure are useful. In some embodiments, the combination of the drugs together is safer or more effective than either drug alone. In some embodiments, the compound disclosed herein and the one or more combination agents have complementary activities that do not adversely affect each other. Such molecules can be present in combination in amounts that are effective for the purpose intended. Such other drug(s) may be administered, by a route and in an amount commonly used therefore, contemporaneously or sequentially with a compound of the present disclosure. When a compound of the present disclosure is used contemporaneously with one or more other drugs, in some embodiments, the agents are administered together in a single pharmaceutical composition in unit dosage form.

Examples of combination agents are GLP-1 drugs. Any GLP-1 inhibitor can be used as the second agent in combination with a compound of the present invention. A “GLP-1 receptor agonist” refers to compounds having GLP-1 receptor activity. Such exemplary compounds include exendins, exendin analogs, exendin agonists, GLP-1(7-37), GLP-1(7-37) analogs, GLP-1(7-37) agonists, and the like. The GLP-1 receptor agonist compounds may optionally be amidated.

The term “exendin” includes naturally occurring (or synthetic versions of naturally occurring) exendin peptides that are found in the salivary secretions of the Gila monster. Exendins of particular interest include exendin-3 and exendin-4. The exendins, exendin analogs, and exendin. agonists for use in the methods described herein may optionally be amidated, and may also be in an acid form, pharmaceutically acceptable salt form, or any other physiologically active form of the molecule.

The combination of a GLP-1 and a GIPR antagonist may achieve the effect of simultaneously improving insulin resistance and reducing excessive fat accumulation (obesity), while lowering blood glucose, and also interfering with lipid metabolism. In this regard, the GLP-1 part may be used to improve glucose tolerance, reduce appetite, lower blood glucose and reduce body weight; whereas the GIPR antagonist compound may be used to reduce the further accumulation of fat, impact reduction in fat mass vs. lean body mass, and improve liver function. The fat reduction effect of the GIPR antagonist and the weight loss effect of the GLP-1 may be used synergistically to treat the non-alcoholic fatty liver disease/non-alcoholic steatohepatitis. This disclosure provides a drug that will benefit patients who have one or more diseases of non-alcoholic fatty liver disease/nonalcoholic steatohepatitis, type 2 diabetes and obesity.

In one embodiment, the GLP-1 receptor agonist is selected from the group consisting of exenatide, liraglutide, lixisenatide, albiglutide, dulaglutide, semaglutide, efpeglenatide, taspoglutide. danuglipron, orforglipron, lotiglipron, PF-06954522, HM15211, L Y3298176, Medi-0382, NN-9924, TTP-054, TTP-273, CT-996, ECC5004, XW004, XW014, MDR-001, ZT002, KN-056, GL0034, GSBR-1290, noiiglutide, RGT-075, TTP-273, HRS-7535, GMA-105, TG103, GZR-18, GX-G6, ecnoglutide, PB-119, QLG2065, beinaglutide, those described in WO2018109607, those described in WO2019239319 (PCT/IB2019/054867 filed Jun. 11, 2019), and those described in WO2019239371 (PCT/IB2019/054961 filed Jun. 13, 2019). In some embodiments, liraglutide is a preferred GLP-1 receptor agonist. Each of the molecules recited above, while depicted in a list, are intended to be recited as individual molecules useful in a combination with the compounds of this invention.

In one embodiment, the molar ratio of a GLP-1 receptor agonist to a GIPR antagonist is from about 1:1 to 1:110, 1:1 to 1:100, 1:1 to 1:75, 1:1 to 1:50, 1:1 to 1:25, 1:1 to 1:10, 1:1 to 1:5, and 1:1. In one embodiment, the molar ratio of a GIPR antagonist to a GLP-1 receptor agonist is from about 1:1 to 1:110, 1:1 to 1:100, 1:1 to 1:75, 1:1 to 1:50, 1:1 to 1:25, 1:1 to 1:10, and 1:1 to 1:5.

Also examples of combination agents are GIPR agonists, which can act as functional GIPR antagonists. Combination therapy with GIPR agonists and antagonists can contribute to additional efficacy in GIPR inhibition. Exemplary GIPR agonist molecules are described in WO2018181864, WO2014192284 and WO2022076503.

In one embodiment, provided herein is a pharmaceutical combination composition comprising: a therapeutically effective amount of a composition comprising:
a first compound, said first compound being a compound of claim 1 or a pharmaceutically acceptable salt of said compound;
a second compound, said second compound being a GLP-1 inhibitor, a GCGR agonist or a GIPR agonist; and a pharmaceutically acceptable excipient.

In one embodiment, provided herein is a pharmaceutical combination composition comprising: a therapeutically effective amount of a composition comprising:
a first compound, said first compound being a compound of claim 1 or a pharmaceutically acceptable salt of said compound;
a second compound, said second compound being a GLP-1 inhibitor; and
a third compound, said third compound being a GCGR agonist or a GIPR agonist; and
a pharmaceutically acceptable excipient.

In one embodiment, the therapeutically effective amounts of the GLP-1 receptor agonist and the GIPR antagonist are administered to the subject simultaneously.

In one embodiment, the therapeutically effective amounts of the GLP-1 receptor agonist and the GIPR antagonist are administered to the subject sequentially.

Any GCGR agonist can be used as the second agent in combination with a compound of the present invention. The combination of a GCGR agonist and a GIPR antagonist may achieve the effect of simultaneously improving insulin resistance and reducing excessive fat accumulation (obesity), while lowering blood glucose, and also interfering with lipid metabolism. Examples of GCGR agonists include antibodies disclosed in U.S. Pat. No. 11,845,802.

In one embodiment, the molar ratio of a GCGR receptor agonist to a GIPR antagonist is from about 1:1 to 1:110, 1:1 to 1:100, 1:1 to 1:75, 1:1 to 1:50, 1:1 to 1:25, 1:1 to 1:10, 1:1 to 1:5, and 1:1. In one embodiment, the molar ratio of a GIPR antagonist to a GCGR receptor agonist is from about 1:1 to 1:110, 1:1 to 1:100, 1:1 to 1:75, 1:1 to 1:50, 1:1 to 1:25, 1:1 to 1:10, and 1:1 to 1:5.

In one embodiment, the therapeutically effective amounts of the GCGR receptor agonist and the GIPR antagonist are administered to the subject simultaneously.

In one embodiment, the therapeutically effective amounts of the GCGR receptor agonist and the GIPR antagonist are administered to the subject sequentially.

The addition of a third hormone to incretin-based therapies, glucagon GCGR agonism has been shown to significantly increase energy expenditure. Thus, the GCGR agonist can be used in combination with the GIPR antagonist and the GLP-1 receptor agonist (GIPR/GLP-1R/GCGR triple agents) in triple therapy. Examples of triple therapy agents include NN9423 (NovoNordisk) and Lilly's LY3437943. [Expert Opinion on Drug Discovery 2023, VOL. 18, NO. 6, 659-669.

Due to the compounds of the invention action on metabolic diseases the compounds may also be beneficially used in combination with other agents such as anti-diabetic agents.

Suitable anti-diabetic agents include insulin, metformin, GLP-1 receptor agonists (described herein above), SGL T2 inhibitors, monoacylglycerol Oacyltransferase inhibitors, phosphodiesterase (PDE)-10 inhibitors, AMPK activators [e.g., ETC-1002 (bempedoic acid)], sulfonylureas (e.g., acetohexamide, chlorpropamide, diabinese, glibenclamide, glipizide, glyburide, glimepiride, gliclazide, glipentide, gliquidone, glisolamide, tolazamide, and tolbutamide), meglitinides, a-amylase inhibitors (e.g., tendamistat, trestatin and AL-3688), an a-glucoside hydrolase inhibitor (e.g., acarbose), a-glucosidase inhibitors (e.g., adiposine, camiglibose, emiglitate, miglitol, voglibose, pradimicin-Q, and salbostatin), PPARy agonists (e.g., balaglitazone, ciglitazone, darglitazone, englitazone, isaglitazone, pioglitazone and rosiglitazone), PPAR a/y agonists (e.g., CLX-0940, GW-1536, GW-1929, GW-2433, KRP—297, L-796449, LR-90, MK-0767 and SB-219994), protein tyrosine phosphatase-1 B (PTP-1 B) inhibitors [e.g., trodusquemine, hyrtiosal extract, and compounds disclosed by Zhang, S. et al., Drug Discovery Today, 12(9/10), 373-381 (2007)], SIRT-1 activators (e.g., resveratrol, GSK2245840 or GSK184072), dipeptidyl peptidase IV (OPP-IV) inhibitors (e.g., those in WO2005116014, sitagliptin, vildagliptin, alogliptin, dutogliptin, linagliptin and saxagliptin), insulin secretagogues, fatty acid oxidation inhibitors, A2 antagonists, c-jun amino-terminal kinase (JNK) inhibitors, glucokinase activators (GKa) such as those described in WO2010103437, WO2010103438, WO2010013161, WO2007122482, TTP-399, TTP-355, TTP-547, AZD1656, ARRY403, MK-0599, TAK-329, AZD5658 or GKM-001, insulin, insulin mimetics, glycogen phosphorylase inhibitors (e.g., GSK1362885), VPAC2 receptor agonists, glucagon receptor modulators such as those described in Demong, D. E. et al., Annual Reports in Medicinal Chemistry 2008, 43, 119-137, GPR119 modulators, particularly agonists, such as those described in WO2010140092, WO2010128425, WO2010128414, WO2010106457, Jones, R. M. et al., Annual Reports in Medicinal Chemistry 2009, 44, 149-170 (e.g., MBX-2982, GSK1292263, APD597 and PSN821), FGF21 derivatives or analogs such as those described in Kharitonenkov, A. et al., Current Opinion in Investigational Drugs 2009, 10(4)359-364, TGR5 (also termed GPBAR1) receptor modulators, particularly agonists, such as those described in Zhong, M., Current Topics in Medicinal Chemistry, 2010, 10(4), 386-396 and INT777, GPR40 agonists, such as those described in Medina, J. C., Annual Reports in Medicinal Chemistry, 2008, 43, 75-85, including but not limited to TAK-875, GPR120 modulators, particularly agonists, high-affinity nicotinic acid receptor (HM74A) activators, and SGL T1 inhibitors, such asGSK1614235. A further representative listing of anti-diabetic agents that can be combined with the compounds of the present invention can be found, for example, at page 28, line 35 through page 30, line 19 of WO2011005611.

Due to the compounds of the invention action on metabolic diseases the compounds may also be beneficially used in combination with other agents. Other combination agents include amylin, NPY2 receptor agents, cannabinoid receptor agents, serotonin receptor agents and MC4R receptor agents which are useful for the treatment of obesity.

In addition, other agents such as lipid modulating agents (HMG-CoA reductase inhibitors such as atorvastatin, pravastatin, pitavastatin, lovastatin, simvastatin, fluvastatin, NK-104 (a.k.a. itavastatin, or nisvastatin or nisbastatin) and ZD-4522 (a.k.a. rosuvastatin, or atavastatin or visastatin) are useful as combination agents.

Due to the compounds of the invention action on metabolic diseases the compounds may also be beneficially used in combination with other cardiovascular agents such as anti-hypertensive agents. Any anti-hypertensive agent can be used as the second agent in such combinations. Such antihypertensive activity is readily determined by those skilled in the art according to standard assays (e.g., blood pressure measurements). Examples include calcium channel blockers (amlodipine besylate), ACE-inhibitors, angiotensin II receptor antagonists, beta-adrenergic receptor blockers, alpha-adrenergic receptor blockers, peripheral vasodilators and diuretics.

Accordingly, the pharmaceutical compositions of the present disclosure also include those that contain one or more other active ingredients, in addition to a compound of the present disclosure. The weight ratio of the compound of the present disclosure to the second active agent may be varied and will depend upon the effective dose of each ingredient. Generally, an effective dose of each will be used. In some embodiments, combination therapy includes therapies in which the compound of the present disclosure and one or more other drugs are administered separately, and in some cases, the two or more agents are administered on different, overlapping schedules. It is also contemplated that when used in combination with one or more other active ingredients, the compounds of the present disclosure and the other active ingredients may be used in lower doses than when each is used singly. In some embodiments, the combination agent is a drug for reduction of symptoms of obesity.

As the present invention has an aspect that relates to the treatment of the disease/conditions described herein with a combination of active ingredients which may be administered jointly, the invention also relates to combining separate pharmaceutical compositions in a single dosage form, such as (but not limited to) a single tablet or capsule, a bilayer or multilayer tablet or capsule, or through the use of segregated components or compartments within a tablet or capsule.

Since the present invention has an aspect that relates to the treatment of the disease/conditions described herein with a combination of active ingredients which may be administered separately, the invention also relates to combining separate pharmaceutical compositions in kit form. The kit comprises two, or more, separate pharmaceutical compositions: a compound of Formula I a prodrug thereof or a salt of such compound or prodrug and a second compound as described above. The kit comprises a means for containing the separate compositions such as a container, a divided bottle or a divided foil packet. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

In some embodiments, provided herein is a kit comprising:
a. a first compound, said first compound being a compound of Formula I, a prodrug thereof, or a pharmaceutically acceptable salt of said compound or of said prodrug and a pharmaceutically acceptable excipient in a first unit dosage form;
b. a second compound, said second compound being a GLP-1 inhibitor, a GCGR agonist or a GIPR agonist and a pharmaceutically acceptable excipient in a second unit dosage form; and
c. means for containing said first and second dosage forms
   wherein the amounts of first and second compounds result in a therapeutic effect.

In some embodiments, the second compound is a GLP-1 inhibitor.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen on which the tablets or capsules so specified should be ingested. Another example of such a memory aid is a calendar printed on the card, e.g., as follows “First Week, Monday, Tuesday, . . . etc. Second Week, Monday, Tuesday, . . . etc. Other variations of memory aids will be readily apparent. A “daily dose” can be a single tablet or capsule or several pills or capsules to be taken on a given day. Also, a daily dose of Formula I compound can consist of one tablet or capsule while a daily dose of the second compound can consist of several tablets or capsules and vice versa. The memory aid should reflect this. In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered microchip memory coupled with a liquid crystal readout, or audible reminder signal which, for example, reads out the date that the last daily dose has been taken and/or reminds one when the next dose is to be taken.

The compounds, pharmaceutical compositions, and methods of the present disclosure can be useful for treating a subject such as, but not limited to, a mammal, a human, a non-human mammal, a domesticated animal (e.g., laboratory animals, household pets, or livestock), a non-domesticated animal (e.g., wildlife), a dog, a cat, a rodent, a mouse, a hamster, a cow, a bird, a chicken, a fish, a pig, a horse, a goat, a sheep, or a rabbit. In preferred embodiments, compounds, pharmaceutical compositions, and methods of the present disclosure are used for treating a human; male or female.

The present invention further comprises use of a compound of Formula I for use as a medicament (Such as a unit dosage tablet or unit dosage capsule). In another embodiment, the present invention comprises the use of a compound of Formula I for the manufacture of a medicament (such as a unit dosage tablet or unit dosage capsule) to treat one or more of the conditions discussed herein.

In practicing the methods described herein, therapeutically effective amounts of the compounds or pharmaceutical compositions described herein can be administered to a subject in need thereof, often for treating and/or preventing a condition or progression thereof. The compounds of this invention and pharmaceutical compositions thereof can affect the physiology of the subject, such as the metabolic system, the immune system, inflammatory response, or other physiologic affect. In addition, the compounds of this invention and pharmaceutical compositions thereof can affect the neuronal (nervous system) based on the incretins role in addictive behavior, Alzheimer's and Parkinson's. A therapeutically effective amount can vary widely depending on the severity of the disease, the age and relative health of the subject, the potency of the compounds used, and other factors.

The Formula I compounds of this invention, their prodrugs and the salts of such compounds and prodrugs (including combinations thereof) are all adapted to therapeutic use as agents that mediate the GIPR in mammals, particularly humans e.g., males, females. For example, these compounds act as GIPR receptor antagonists and thus are useful for the treatment of the various conditions (e.g., those described herein) in which such action is implicated.

Given the positive correlation between mediation of the GIPR receptor with the number of physiological effects in tissues—including promotion of fat storage in adipocytes, promotion of pancreatic islet cell function and beta-cell survival, glucose-dependent insulin secretion, and that GIPR is highly expressed in a number of tissues (including the pancreas, gut, adipose tissue, heart, pituitary, adrenal cortex, and brain) (Usdin et al., Endocrinology. 1993, 133:2861-2870), GIPR antagonists are useful for the treatment of glucose metabolism (e.g. Type 2 diabetes, elevated glucose levels, elevated insulin levels, dyslipidemia, metabolic syndrome (Syndrome X or insulin resistance syndrome), glucosuria, metabolic acidosis, Type 1 diabetes, obesity and conditions exacerbated by obesity), pancreatitis, cirrhosis and Turner's & Cushing's syndrome.

Preferred disease/conditions include obesity and type II diabetes. An especially preferred disease/condition is obesity.

Through the compounds of this invention capacity to treat obesity they also have a treatment impact on cardiovascular diseases, including coronary artery disease, myocardial infarction, heart failure. stroke, venous thromboembolic disease, and pulmonary hypertension. Circulation 2021 143:e984-e1010.

Through the compounds of this invention capacity to treat obesity they also have a treatment impact on cancer including post-menopausal breast, colorectal, endometrial, esophageal, pancreatic, renal, liver, stomach, gallbladder, ovarian, thyroid, multiple myeloma, and meningioma. Other cancers associated with obesity include cancers of the mouth, pharynx and larynx, prostate, and male breast, as well as diffuse large B-cell lymphoma. A high risk associated with obesity is shown for three of the most challenging cancers to treat—pancreatic, esophageal, and gallbladder—as well as the two most prevalent malignancies—breast and colorectal cancer. Cancers 2023, 15,485

Through the compounds of this invention capacity to treat obesity they also have a treatment impact on obstructive sleep apnea, and associated conditions.

Through the compounds of this invention capacity to treat obesity they also have a treatment impact on osteoarthritis and associated disease/conditions.

Further the compounds of this invention can be used to treat any GIPR-related condition, disease, or disorder including the following: diabetes [e.g. Type 1 diabetes mellitus (T1 D), Type 2 diabetes mellitus (T2DM), including pre-diabetes], idiopathic T1 D (Type 1 b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, depression, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug).

### EXAMPLES

As can be appreciated by the skilled artisan, the representative examples (below) are not intended to comprise a comprehensive list of all means by which the compounds described and claimed in this application may be synthesized. Further methods will be evident to those of ordinary skill in the art. Additionally, the various synthetic steps described in the examples may be performed in an alternate sequence or order to give the desired compounds. The disclosure further encompasses “intermediate” compounds, including structures produced from the synthetic procedures described, whether isolated or generated in-situ and not isolated, prior to obtaining the finally desired compound. These intermediates are included in the scope of this disclosure. Exemplary embodiments of such intermediate compounds are set forth in the Examples below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). The starting materials and the intermediates, and the final products of a reaction may be isolated and purified if desired using conventional techniques, including but not limited to filtration, distillation, crystallization, chromatography and the like. Such materials may be characterized using conventional means, including physical constants and spectral data.

The chemical names may be generated using the ChemDraw Professional, for example ChemDraw Professional Version 22.2.0.3348, 23.0.1.11 or 23.1.1.3. Other software naming programs such as Marvin JS 21.19.0 software naming programs may also be used.

The following abbreviations may be used in throughout the text: AIBN=azobisisobutyronitrile, BINAP=2,2′-bis(diphenylphosphino)-1,1′-binaphthyl, Boc=tert-butyloxycarbonyl, BSA=Bovine Serum Albumin, bpy or bipy=bipyridine, BTMG=2-tert-Butyl-1,1,3,3-tetramethylguanidine, cAMP=Cyclic adenosine monophosphate, Cbz=(benzyloxycarbonyl, CDI=carbonyldiimidazole, DMA=Dimethylacetamide, DMSO=dimethyl sulfoxide, ex=excitation; em=emission, LCMS=liquid chromatography-mass spectrometry, ESI=Electrospray Ionization, M+H=a unit higher than the monoisotopic mass of the uncharged molecule, HPLC=High pressure liquid chromatography, Prep-HPLC=preparatory scale HPLC, AcOH=acetic acid, ACN or MeCN=acetonitrile, PE=petroleum ether, EA or EtOAc=ethyl acetate, EDCI=1-ethyl-3-(3-dimethylaminopropyl)carbodiimide, dtbbpy=4,4′-Di-tert-butyl-2,2′-dipyridyl, DBU=1,8-diazabicyclo[5.4.0]undec-7-ene, DEA=diethanolamine, DEAD=diethylazodicarboxylate, DIAD=Diisopropyl azodicarboxylate, DIEA=N,N-diisopropylethylamine, DMAP=4-Dimethylaminopyridine, DME=dimethyl ether, DMSO=dimethyl sulfoxide, DMF=N, N-dimethylacetamide, DCM=dichloromethane, DPPA=diphenylphosphoryl azide, dppf=1,1′-Bis(diphenylphosphino)ferrocene, EtOH=ethanol, FA=formic acid, MeOH=methanol, MtBE/MTBE=methyl tert-butyl ether, TEA or Et₃N=triethylamine, TFA=trifluoroacetic acid, GOI=genes of interest, HATU=hexafluorophosphate azabenzotriazole tetramethyl uronium, HFIP=hexafluoroisopropanol, HMDS=hexamethyldisilazane, HCl=hydrochloric acid, HBSS=Hanks' Balanced Salt Solution, HEPES=4-(2-hydroxyethyl)-1-piperazineethane sulfonic acid, HOAt=1-hydroxy-7-azabenzotriazole, HOBt=1-hydroxybenzotriazole, SFC=Supercritical fluid chromatography, TCFH=N,N,N′,N′-tetramethylchloroformamidinium hexafluorophosphate, THF=tetrahydrofuran, TLC=thin layer chromatography, TMG=tetramethylguanidine, T3P=propanephosphonic acid cyclic anhydride, Oac=acetate, m-CPBA=meta-chloroperoxybenzoic acid, NiBr₂·Bipy=(2, 2′-bipyridine) nickel (II) dibromide, NMM=N-methylmorpholine, NMI=1-Methylimidazole, POCl₃=Phosphoryl chloride, py=pyridine, Pd/C=palladium on carbon, HTRF—Homogeneous Time-Resolved Fluorescence, IBMX=Isobutylmethylxanthine, RT=retention time, h=hour, hrs=hours, aq.=aqueous, min.=minute, sat.=saturated, equiv=equivalent, e.e.=enantiomeric excess, UV=ultraviolet, 4-CZIPN=tetrakis(9H-carbazol-9-yl)benzene-1,4-dicarbonitrile, 4-DPAIPN=1,3-Dicyano-2,4,5,6-tetrakis(diphenylamino)-benzene, XPhos Pd G3=2-Dicyclohexylphosphino-2′,4′,6′-triisopropyl-1,1′-biphenyl)[2-(2′-amino-1,1′-biphenyl)]palladium(II) methanesulfonate, Ir[dF(CF₃)ppy]₂(dtbpy))PF₆=([4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate); GIP=Gastric inhibitory polypeptide or glucose-dependent insulinotropic polypeptide; GIPR=Gastric inhibitory polypeptide receptor; GIPR=Gastric inhibitory polypeptide receptor and/or glucose-dependent insulinotropic polypeptide receptor.

### Compound Examples

The following compounds are provided as examples of this invention.

### Synthesis and Characterization Examples

For certain compounds disclosed herein the absolute stereochemistry has not been independently confirmed. Thus, in some instances, the absolute stereo configurations of one or more chiral centers are arbitrarily assigned (e.g., stereochemistry of one chiral center is known and remaining chiral centers arbitrarily assigned). Accordingly, the enantiomers or diastereomers are identified by their respective properties, for example, retention times on a prep chiral HPLC, chiral SFC, NMR shift or optical rotation or its biological activities (e.g., as described further in the Examples). Thus, should the stereochemistry assigned to any compound or compounds ultimately be proven incorrect, then the analytical data (e.g., prep chiral HPLC, chiral SFC, NMR shift or optical rotation or biological activity) associated with each compound is determinative of the actual identity of the compound. In addition, in light of such corrected stereochemical designation appropriate adjustments to the stereochemistry identification contained in the description, examples, tables and claims should be adjusted as needed by one skilled in the art.

Chiral analytical separation methods (e.g., supercritical fluid chromatography (SFC), and high performance liquid chromatography (HPLC)) used in the following synthetic examples are summarized in Table A below. These methods were used to identify a single compound/stereoisomer from a mixture of chiral compounds based on a peak retention time.

TABLE A

| | | | | | Column | Back | |
|---|---|---|---|---|---|---|---|
| Method | Column | Mobile Phase | Flow Type | Flow Rate | Temp. | Pressure | |
| A | CHIRALPAK IC-3, | A: Hexanes | Isocratic: 30% | 1.67 mL/min | Ambient | | |
| | 4.6 × 50 mm, 3 μM | (0.1% TFA) | B | | | | |
| | | B: EtOH | | | | | |
| B | CHIRAL Cellulose-SB, | A: MtBE (0.1% | Isocratic: 15% | 1.67 mL/min | Ambient | | |
| | 4.6 × 50 mm, | TFA) B: MeOH | B | | | | |
| | 3 μM | | | | | | |
| C | (R, R)-Whelk-O1 | A: CO₂ B: | Isocratic: 50% | 4.0 mL/min | 35° C. | 1500 | psi |
| | (4.6 × 100 mm, 3.5 | MeOH (0.1% | B | | | | |
| | μm) | DEA) | | | | | |
| D | CHIRALPAK AD- | A: CO₂ B: | Gradient | 2.0 mL/min | 35° C. | 1500 | psi |
| | 3, | MeOH (0.1% | (B %): 10% to | | | | |
| | 3.0 × 100 mm, 3 | DEA) | 50% in 4.0 | | | | |
| | μM | | min, hold 2.0 | | | | |
| | | | min at 50% | | | | |
| E | CHIRALART | A: CO₂ B: | Gradient | 4.0 mL/min | 35° C. | 1500 | psi |
| | Cellulose-SB, 4.6 × | MeOH (0.1% | (B %): 10% to | | | | |
| | 50 mm, 3 μM | DEA) | 50% in 2.0 | | | | |
| | | | min, hold 1.0 | | | | |
| | | | min at 50% | | | | |
| F | CHIRALART | A: CO₂ B: | Gradient | 2.0 mL/min | 35° C. | 1500 | psi |
| | Cellulose-SB, 4.6 × | MeOH (0.1% | (B %): 10% to | | | | |
| | 50 mm, 3 μM | DEA) | 50% in 4.0 | | | | |
| | | | min, hold 2.0 | | | | |
| | | | min at 50% | | | | |
| G | CHIRALPAK IK- | A: Hexanes | Isocratic: 30% | 1.67 mL/min | Ambient | | |
| | 3, | (0.1% TFA) B: | B | | | | |
| | 4.6 × 50 mm, 3 μM | EtOH | | | | | |
| I | CHIRALPAK IK- | A: Hexanes | Isocratic: 50% | 1.67 mL/min | Ambient | | |
| | 3, | (0.1% TFA) B: | B | | | | |
| | 4.6 × 50 mm, 3 μM | EtOH | | | | | |
| J | CHIRAL Cellulose-SB, | A: MtBE (0.1% | Isocratic: 10% | 1.67 mL/min | Ambient | | |
| | 4.6 × 50 mm, | TFA) B: MeOH | B | | | | |
| | 3 μM | | | | | | |
| K | Cellulose SZ, 4.6 × | A: Hexanes | Isocratic: 30% | 1.67 mL/min | Ambient | | |
| | 50 mm, 3 μM | (0.1% TFA) B: | B | | | | |
| | | EtOH | | | | | |
| L | CHIRAL Cellulose-SB, | A: Hexanes | Isocratic: 20% | 1.67 mL/min | Ambient | | |
| | 4.6 × 50 mm, | (0.1% TFA) B: | B | | | | |
| | 3 μM | EtOH | | | | | |
| M | CHIRAL Cellulose-SB, | A: Hexanes | Isocratic: 30% | 1.67 mL/min | Ambient | | |
| | 4.6 × 50 mm, | (0.1% TFA) B: | B | | | | |
| | 3 μM | EtOH | | | | | |
| N | CHIRALPAK IA- | A: MtBE (0.1% | Isocratic: 10% | 1.67 mL/min | Ambient | | |
| | 3, | TFA) B: MeOH | B | | | | |
| | 4.6 × 50 mm, 3 μM | | | | | | |
| O | CHIRALPAK IK- | A: Hexanes | Isocratic: 10% | 1.67 mL/min | Ambient | | |
| | 3, | (0.1% TFA) B: | B | | | | |
| | 4.6 × 50 mm, 3 μM | EtOH | | | | | |
| P | CHIRALPAK IK- | A: Hexanes | Isocratic: 20% | 1.67 mL/min | Ambient | | |
| | 3, | (0.1% TFA) B: | B | | | | |
| | 4.6 × 50 mm, 3 μM | EtOH | | | | | |
| Q | CHIRALPAK IE-3, | A: Hexanes | Isocratic: 40% | 1.67 mL/min | Ambient | | |
| | 4.6 × 50 mm, 3 μM | (0.1% TFA) B: | B | | | | |
| | | EtOH | | | | | |
| R | Cellulose SZ, 4.6 × | A: Hexanes | Isocratic: 50% | 1.67 mL/min | Ambient | | |
| | 50 mm, 3 μM | (0.1% TFA) B: | B | | | | |
| | | EtOH | | | | | |
| S | CHIRAL Cellulose-SB, | A: 1:1 | Isocratic: 10% | 1.67 mL/min | Ambient | | |
| | 4.6 × 50 mm, | MtBE:Hexanes | B | | | | |
| | 3 μM | (0.1% TFA) B: | | | | | |
| | | EtOH | | | | | |
| T | CHIRALPAK ID- | A: 1:1 | Isocratic: 10% | 1.67 mL/min | Ambient | | |
| | 3, | MtBE:Hexanes | B | | | | |
| | 4.6 × 50 mm, 3 μM | (0.1% TFA) B: | | | | | |
| | | MeOH | | | | | |
| U | CHIRALPAK IC-3, | A: MtBE (0.1% | Isocratic: 10% | 1.67 mL/min | Ambient | | |
| | 4.6 × 50 mm, 3 μM | TFA) B: MeOH | B | | | | |
| V | CHIRAL Cellulose-SB, | A: 1:1 | Isocratic: 10% | 1.67 mL/min | Ambient | | |
| | 4.6 × 50 mm, | MtBE:Hexanes | B | | | | |
| | 3 μM | (0.1% TFA) B: | | | | | |
| | | MeOH | | | | | |
| W | CHIRALPAK IC-3, | A: Hexanes | Isocratic: 20% | 1.67 mL/min | Ambient | | |
| | 4.6 × 50 mm, 3 μM | (0.1% TFA) B: | B | | | | |
| | | EtOH | | | | | |
| X | CHIRALPAK ID- | A: CO₂ B: IPA | Gradient | 2.0 mL/min | 35° C. | 100 | bar |
| | 3, | (20 mM NH₃) | (B %): 10% to | | | | |
| | 3.0 × 50 mm, 3 μM | | 50% in 2.0 | | | | |
| | | | min, hold 1.0 | | | | |
| | | | min at 50% | | | | |
| Y | CHIRALPAK AD- | A: CO₂ B: | Isocratic: 25% | 2.0 mL/min | 35° C. | 100 | bar |
| | 3, | MeOH (0.1% | B | | | | |
| | 3.0 × 100 mm, 3 | DEA) | | | | | |
| | μM | | | | | | |
| Z | CHIRALPAK IC-3, | A: Hexanes | Isocratic: 40% | 1.67 mL/min | Ambient | | |
| | 4.6 × 50 mm, 3 μM | (0.1% TFA) B: | B | | | | |
| | | EtOH | | | | | |
| AA | CHIRALPAK IC-3, | A: Hexanes | Isocratic: 30% | 1.67 mL/min | Ambient | | |
| | 4.6 × 50 mm, 3 μM | (0.1% TFA) B: | B | | | | |
| | | EtOH | | | | | |
| AB | CHIRALPAK IG- | A: Hexanes | Isocratic: 50% | 1.67 mL/min | Ambient | | |
| | 3, | (0.1% TFA) B: | B | | | | |
| | 4.6 × 50 mm, 3 μM | EtOH | | | | | |
| AC | CHIRAL ART | A: CO₂ B: | Gradient | 2.0 mL/min | 35° C. | 110 | bar |
| | Cellulose-SB, 3.0 × | MeOH (0.1% | (B %): 10% to | | | | |
| | 50 mm, 3 μM | DEA) | 50% in 2.0 | | | | |
| | | | min, hold 1.0 | | | | |
| | | | min at 50% | | | | |
| AD | CHIRALPAK IK- | A: CO₂ B: | Gradient | 2.0 mL/min | 35° C. | 100 | bar |
| | 3, 3.0 × 50 mm, 3 | MeOH (0.1% | (B %): 10% to | | | | |
| | μM | DEA) | 50% in 2.0 | | | | |
| | | | min, hold 1.0 | | | | |
| | | | min at 50% | | | | |
| AE | Opti-Chiral C3-3, | A: CO₂ B: | Gradient | 2.0 mL/min | 35° C. | 100 | bar |
| | 3.0 × 50 mm, 3 μM | MeOH (0.1% | (B %): 10% to | | | | |
| | | DEA) | 50% in 2.0 | | | | |
| | | | min, hold 1.0 | | | | |
| | | | min at 50% | | | | |
| AE | CHIRALPAK ID- | A: MtBE (0.1% | Isocratic: 20% | 1.67 mL/min | Ambient | | |
| | 3, | DEA) B: MeOH | B | | | | |
| | 4.6 × 50 mm, 3 μM | | | | | | |
| AF | CHIRALPAK IA- | A: Hexanes | Isocratic: 30% | 1.67 mL/min | Ambient | | |
| | 3, | (0.1% TFA) B: | B | | | | |
| | 4.6 × 50 mm, 3 μM | EtOH | | | | | |
| AG | CHIRALPAK ID- | A: Hexanes | Isocratic: 50% | 1.67 mL/min | Ambient | | |
| | 3, | (0.1% TFA) B: | B | | | | |
| | 4.6 × 50 mm, 3 μM | EtOH | | | | | |
| AH | CHIRAL NX (2), | A: Hexanes | Isocratic: 30% | 1.67 mL/min | Ambient | | |
| | 4.6 × 50 mm, 3 μM | (0.1% TFA) B: | B | | | | |
| | | EtOH | | | | | |
| AI | CHIRALPAK IE-3, | A: MtBE (0.1% | Isocratic: 10% | 1.0 mL/min | Ambient | | |
| | 4.6 × 50 mm, 3 μM | TFA) B: MeOH | B | | | | |
| AJ | CHIRAL NX (2), | A: Hexanes | Isocratic: 50% | 1.0 mL/min | Ambient | | |
| | 4.6 × 50 mm, 3 μM | (0.1% TFA) B: | B | | | | |
| | | EtOH | | | | | |
| AK | CHIRALPAK IK- | A: Hexanes | Isocratic: 50% | 1.67 mL/min | Ambient | | |
| | 3, | (0.1% DEA) B: | B | | | | |
| | 4.6 × 50 mm, 3 μM | EtOH | | | | | |
| AL | CHIRALPAK IK- | A: Hexanes | Isocratic: 20% | 1.67 mL/min | Ambient | | |
| | 3, | (0.1% DEA) B: | B | | | | |
| | 4.6 × 50 mm, 3 μM | EtOH | | | | | |
| AM | CHIRALPAK IK- | A: Hexanes | Isocratic: 30% | 1.67 mL/min | Ambient | | |
| | 3, | (0.1% DEA) B: | B | | | | |
| | 4.6 × 50 mm, 3 μM | EtOH | | | | | |
| AN | CHIRAL ART | A: Hexanes | Isocratic: 30% | 1.67 mL/min | Ambient | | |
| | Amylose-C, 4.6 × | (0.1% TFA) B: | B | | | | |
| | 50 mm, 3 μM | EtOH:MeOH | | | | | |
| | | (1:1) | | | | | |
| AO | Lux 3μ Cellulose-2, | A: Hexanes | Isocratic: 30% | 1.67 mL/min | Ambient | | |
| | 4.6 × 50 mm, 3 μM | (0.1% TFA) B: | B | | | | |
| | | EtOH | | | | | |
| AP | CHIRALPAK IG- | A: MtBE (0.1% | Isocratic: 30% | 1.67 mL/min | Ambient | | |
| | 3, | TFA) B: MeOH | B | | | | |
| | 4.6 × 50 mm, 3 μM | | | | | | |
| AQ | CHIRALPAK ID- | A: Hexanes | Isocratic: 30% | 1.67 mL/min | Ambient | | |
| | 3, | (0.1% TFA) B: | B | | | | |
| | 4.6 × 50 mm, 3 μM | EtOH | | | | | |
| AR | CHIRALPAK ID- | A: Hexanes | Isocratic: 30% | 1.67 mL/min | Ambient | | |
| | 3, | (0.1% TFA) B: | B | | | | |
| | 4.6 × 50 mm, 3 μM | IPA | | | | | |
| AS | Enantiocel A4-3, | A: Hexanes | Isocratic: 30% | 1.67 mL/min | Ambient | | |
| | 4.6 × 50 mm, 3 μM | (0.1% DEA) B: | B | | | | |
| | | EtOH:MeCN | | | | | |
| | | (5:1) | | | | | |
| AT | CHIRALPAK IH- | A: Hexanes | Isocratic: 30% | 1.67 mL/min | Ambient | | |
| | 3, | (0.1% DEA) B: | B | | | | |
| | 4.6 × 50 mm, 3 μM | EtOH | | | | | |
| AU | CHIRALPAK ID- | A: 1:1 | Isocratic: 10% | 1.67 mL/min | Ambient | | |
| | 3, | MtBE:Hexanes | B | | | | |
| | 4.6 × 50 mm, 3 μM | (0.1% TFA) B: | | | | | |
| | | EtOH | | | | | |
| AV | CHIRALPAK IA- | A: 1:1 | Isocratic: 30% | 1.67 mL/min | Ambient | | |
| | 3, | MtBE:Hexanes (0.1% | B | | | | |
| | 4.6 × 50 mm, 3 μM | TFA) B: MeOH | | | | | |
| AW | CHIRALPAK IC-3, | A: Hexanes | Isocratic: 10% | 1.67 mL/min | Ambient | | |
| | 4.6 × 50 mm, 3 μM | (0.1% TFA) B: | B | | | | |
| | | EtOH | | | | | |
| AX | CHIRALPAK IC-3, | A: Hexanes | Isocratic: 40% | 1.67 mL/min | Ambient | | |
| | 4.6 × 50 mm, 3 μM | (0.1% DEA) B: | B | | | | |
| | | EtOH | | | | | |
| AY | Cellulose-SB, 4.6 × | A: Hexanes | Isocratic: 50% | 1.0 mL/min | Ambient | | |
| | 50 mm, 3 μM | (0.1% TFA) B: | B | | | | |
| | | EtOH | | | | | |
| AZ | CHIRAL Cellulose-SB, | A: 1:1 | Isocratic: 20% | 1.67 mL/min | Ambient | | |
| | 4.6 × 50 mm, | MtBE:Hexanes | B | | | | |
| | 3 μM | (0.1% TFA) B: | | | | | |
| | | MeOH | | | | | |
| BA | CHIRAL Cellulose-SB, | A: 1:1 | Isocratic: 30% | 1.67 mL/min | Ambient | | |
| | 4.6 × 50 mm, | MtBE:Hexanes | B | | | | |
| | 3 μM | (0.1% TFA) B: | | | | | |
| | | MeOH | | | | | |
| BB | CHIRALPAK IK- | A: Hexanes | Isocratic: 30% | 1.67 mL/min | Ambient | | |
| | 3, | (0.1% TFA) B: | B | | | | |
| | 4.6 × 50 mm, 3 μM | IPA | | | | | |
| BC | Cellulose-SB, 4.6 × | A: Hexanes | Isocratic: 20% | 1.0 mL/min | Ambient | | |
| | 50 mm, 3 μM | (0.1% TFA) B: | B | | | | |
| | | EtOH | | | | | |
| BD | CHIRALPAK IA- | A: 1:1 | Isocratic: 20% | 1.67 mL/min | Ambient | | |
| | 3, | MtBE:Hexanes (0.1% | B | | | | |
| | 4.6 × 50 mm, 3 μM | TFA) B: MeOH | | | | | |
| BE | CHIRALPAK IM- | A: Hexanes | Isocratic: 50% | 1.67 mL/min | Ambient | | |
| | 3, | (0.1% TFA) B: | B | | | | |
| | 4.6 × 50 mm, 3 μM | EtOH | | | | | |
| BF | CHIRALPAK IA- | A: 1:1 | Isocratic: 40% | 1.67 mL/min | Ambient | | |
| | 3, | MtBE:Hexanes (0.1% | B | | | | |
| | 4.6 × 50 mm, 3 μM | TFA) B: MeOH | | | | | |
| BG | CHIRALPAK IC-3, | A: Hexanes | Isocratic: 50% | 1.67 mL/min | Ambient | | |
| | 4.6 × 50 mm, 3 μM | (0.1% TFA) B: | B | | | | |
| | | EtOH | | | | | |
| BH | CHIRALPAK IK- | A: MtBE (0.1% | Isocratic: 50% | 1.67 mL/min | Ambient | | |
| | 3, | TFA) B: MeOH | B | | | | |
| | 4.6 × 50 mm, 3 μM | | | | | | |
| BI | Cellulose-SB, 4.6 × | A: Hexanes | Isocratic: 50% | 1.67 mL/min | Ambient | | |
| | 50 mm, 3 μM | (0.1% TFA) B: | B | | | | |
| | | EtOH | | | | | |
| BJ | CHIRALPAK IM- | A: MtBE (0.1% | Isocratic: 30% | 1.67 mL/min | Ambient | | |
| | 3, | TFA) B: MeOH | B | | | | |
| | 4.6 × 50 mm, 3 μM | | | | | | |
| BK | Cellulose SZ, 4.6 × | A: MtBE (0.1% | Isocratic: 50% | 1.67 mL/min | Ambient | | |
| | 50 mm, 3 μM | TFA) B: MeOH | B | | | | |
| BL | CHIRALPAK IM- | A: MtBE (0.1% | Isocratic: 50% | 1.67 mL/min | Ambient | | |
| | 3, | TFA) B: MeOH | B | | | | |
| | 4.6 × 50 mm, 3 μM | | | | | | |
| BM | CHIRALPAK IM- | A: Hexanes | Isocratic: 30% | 1.67 mL/min | Ambient | | |
| | 3, | (0.1% TFA) B: | B | | | | |
| | 4.6 × 50 mm, 3 μM | EtOH | | | | | |
| BN | Lux 3μ Cellulose-2, | A: Hexanes | Isocratic: 50% | 1.0 mL/min | Ambient | | |
| | 4.6 × 50 mm, 3 μM | (0.1% TFA) B: | B | | | | |
| | | EtOH | | | | | |
| BO | Cellulose SZ, 4.6 × | A: MtBE (0.1% | Isocratic: 30% | 1.67 mL/min | Ambient | | |
| | 50 mm, 3 μM | DEA) B: MeOH | B | | | | |
| BP | CHIRALPAK IK- | A: MtBE (0.1% | Isocratic: 30% | 1.67 mL/min | Ambient | | |
| | 3, | TFA) B: MeOH | B | | | | |
| | 4.6 × 50 mm, 3 μM | | | | | | |
| BQ | Cellulose SB, 4.6 × | A: Hexanes | Isocratic: 30% | 1.67 mL/min | Ambient | | |
| | 50 mm, 3 μM | (0.1% TFA) B: | B | | | | |
| | | EtOH | | | | | |
| BR | CHIRALPAK IC-3, | A: MtBE (0.1% | Isocratic: 20% | 1.67 mL/min | Ambient | | |
| | 4.6 × 50 mm, 3 μM | TFA) B: MeOH | B | | | | |
| BS | Cellulose SB, 4.6 × | A: Hexanes | Isocratic: 30% | 1.0 mL/min | Ambient | | |
| | 50 mm, 3 μM | (0.1% TFA) B: | B | | | | |
| | | EtOH | | | | | |
| BT | CHIRALPAK IG- | A: Hexanes | Isocratic: 50% | 1.67 mL/min | Ambient | | |
| | 3, | (0.1% TFA) B: | B | | | | |
| | 4.6 × 50 mm, 3 μM | IPA | | | | | |
| BU | Cellulose SZ, 4.6 × | A: MtBE (0.1% | Isocratic: 50% | 1.0 mL/min | Ambient | | |
| | 50 mm, 3 μM | TFA) B: MeOH | B | | | | |
| BV | CHIRALPAK ID- | A: 1:1 | Isocratic: 10% | 1.0 mL/min | Ambient | | |
| | 3, | MtBE:Hexanes | B | | | | |
| | 4.6 × 50 mm, 3 μM | (0.1% TFA) B: | | | | | |
| | | EtOH | | | | | |
| BW | Cellulose SB, 4.6 × | A: Hexanes | Isocratic: 30% | 1.0 mL/min | Ambient | | |
| | 50 mm, 3 μM | (0.1% TFA) B: | B | | | | |
| | | 5:1 EtOH:MeCN | | | | | |
| BX | Lux 3μ Cellulose-2, | A: Hexanes | Isocratic: 30% | 1.0 mL/min | Ambient | | |
| | 4.6 × 50 mm, 3 μM | (0.1% TFA) B: | B | | | | |
| | | EtOH | | | | | |

### Procedures for Preparing Intermediate Compounds Used in the Examples

### Preparation of methyl 4-(5-fluoro-6-(pyrrolidin-2-yl)pyridin-3-yl)benzoate

### Step 1: methyl 4-(6-chloro-5-fluoropyridin-3-yl)benzoate

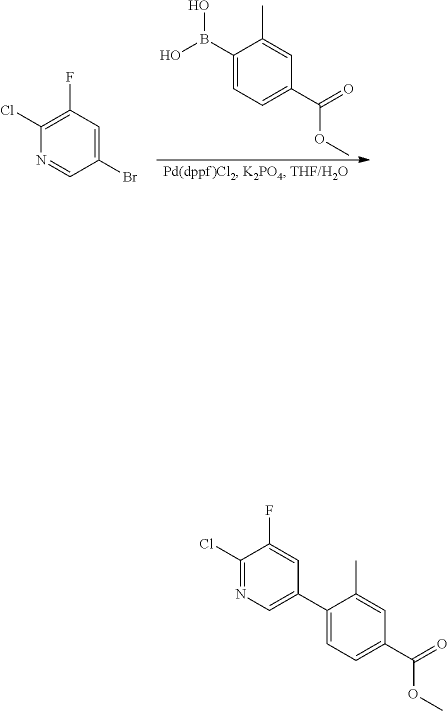 [SMILES: Fc1cc(Br)cnc1Cl ; COC(=O)c1ccc(B(O)O)c(C)c1 ; COC(=O)c1ccc(-c2cnc(Cl)c(F)c2)c(C)c1]

A solution of 5-bromo-2-chloro-3-fluoropyridine (10 g, 47.5 mmol) and 4-(methoxycarbonyl)phenylboronic acid (8.55 g, 47.5 mmol), K₃PO₄ (30.3 g, 143 mmol) and Pd(dppf)Cl₂ (3.48 g, 4.75 mmol) in THF (30 mL) and H₂O (7.5 mL) was stirred at 60° C. for 2 hours under nitrogen atmosphere. Upon completion, the resulting mixture was concentrated under reduced pressure. The crude residue was directly purified by silica gel column chromatography, eluted with PE/EA (6:1) to afford methyl 4-(6-chloro-5-fluoropyridin-3-yl)benzoate (5.6 g, 44%) as a white solid. LCMS (ESI) [M+H]⁺: 266.0.

### Step 2: tert-butyl 2-(3-fluoro-5-(4-(methoxycarbonyl)phenyl)pyridin-2-yl)pyrrolidine-1-carboxylate

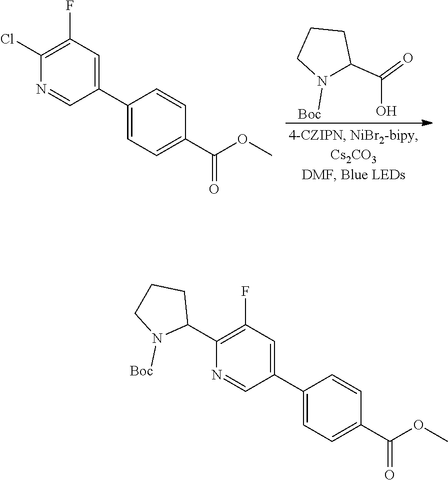 [SMILES: COC(=O)c1ccc(-c2cnc(Cl)c(F)c2)cc1 ; CC(C)(C)OC(=O)N1CCCC1C(=O)O ; COC(=O)c1ccc(-c2cnc(C3CCCN3C(=O)OC(C)(C)C)c(F)c2)cc1]

A solution of methyl 4-(6-chloro-5-fluoropyridin-3-yl)benzoate (5.6 g, 21.1 mmol), Cs₂CO₃ (20.4 g, 63.2 mmol), tetrakis(9H-carbazol-9-yl)benzene-1,4-dicarbonitrile (416 mg, 0.527 mmol) and NiBr₂-bipy (790 mg, 2.11 mmol) in DMF (16 mL) was treated with (tert-butoxycarbonyl)proline (6.81 g, 31.6 mmol) at room temperature under nitrogen atmosphere. The resulting mixture was stirred under irradiation at 450 nm with blue LEDs for 24 hours. Upon completion, the resulting mixture was diluted with H₂O (8 mL) and extracted with ethyl acetate (3×40 mL). The combined organic layers were washed with brine (3×50 mL), dried over anhydrous Na₂SO₄, filtered, and concentrated under reduced pressure. The crude residue was purified by silica gel column chromatography, eluted with PE/EA (5:1) to afford tert-butyl 2-(3-fluoro-5-(4-(methoxycarbonyl)phenyl)pyridin-2-yl)pyrrolidine-1-carboxylate (2.00 g, 24%) as a brown oil. LCMS (ESI) [M+H]⁺: 401.2.

### Step 3: methyl 4-(5-fluoro-6-(pyrrolidin-2-yl)pyridin-3-yl)benzoate

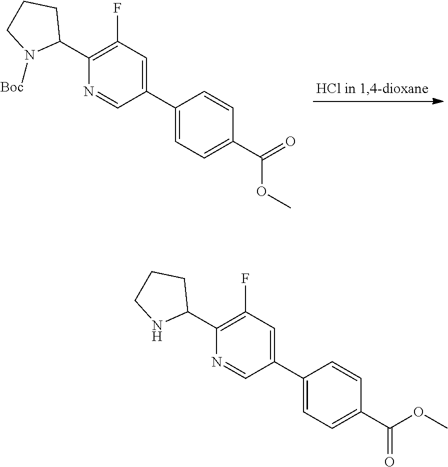 [SMILES: COC(=O)c1ccc(-c2cnc(C3CCCN3C(=O)OC(C)(C)C)c(F)c2)cc1 ; COC(=O)c1ccc(-c2cnc(C3CCCN3)c(F)c2)cc1]

A solution of tert-butyl 2-(3-fluoro-5-(4-(methoxycarbonyl)phenyl)pyridin-2-yl)pyrrolidine-1-carboxylate (2.00 g, 5.00 mmol) in hydrochloric acid (8 mL, 32 mmol, 4.0 M in 1,4-dioxane) was stirred at room temperature for 2 hours under nitrogen atmosphere. Upon completion, the resulting mixture was concentrated under reduced pressure. The crude residue was purified by reversed-phase flash chromatography (conditions: column: C18 silica gel; mobile phase A: water (0.1% formic acid); mobile phase B: MeCN; gradient (B %): 0% to 100% over 30 min; detector: UV 254 nm) to afford methyl 4-(5-fluoro-6-(pyrrolidin-2-yl)pyridin-3-yl)benzoate (1.05 g, 70%) as a yellow green solid. LCMS (ESI) [M+H]⁺: 301.1.

### Preparation of 4-(5-fluoro-6-(pyrrolidin-2-yl)pyridin-3-yl)benzoic acid

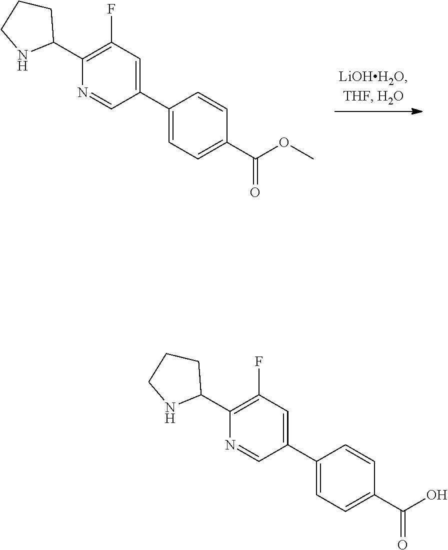 [SMILES: COC(=O)c1ccc(-c2cnc(C3CCCN3)c(F)c2)cc1 ; O=C(O)c1ccc(-c2cnc(C3CCCN3)c(F)c2)cc1]

To a mixture of methyl 4-(5-fluoro-6-(pyrrolidin-2-yl)pyridin-3-yl)benzoate (1.00 g, 3.33 mmol) in THF (20 mL) were added LiOH monohydrate (420 mg, 1.00 mmol) and H₂O (10 mL). The resulting mixture was stirred at room temperature for 2 hours under an atmosphere of nitrogen. Upon completion, the mixture was acidified to pH 3 with HCl (1 M in water) and the resulting mixture concentrated under reduced pressure. The crude residue was directly purified by reversed phase flash chromatography (conditions: column: C18 silica gel; mobile phase A: Water (0.1% FA), mobile phase B: MeCN; Gradient (B %): 0 to 100% in 25 min; detector: UV 254 nm) to afford 4-(5-fluoro-6-(pyrrolidin-2-yl)pyridin-3-yl)benzoic acid (750 mg, 79%) as an off-white solid. LCMS [M+H]⁺: 286.1

### Preparation of 5-(4-(3,3-dimethylazetidine-2-carboxamido)phenyl)-6-methylpicolinic acid

### Step 1: methyl 3,3-dimethyl-2-(picolinamido)butanoate

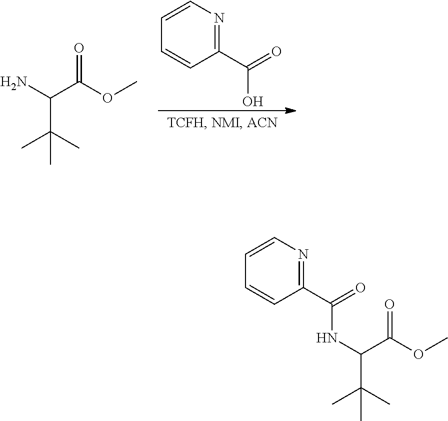

A solution of picolinic acid (20.3 g, 165 mmol), TCFH (77.3 g, 275 mmol), NMI (56.5 g, 689 mmol) and methyl 2-amino-3,3-dimethylbutanoate (20.0 g, 138 mmol) in ACN (100 mL) was stirred at room temperature for 1 hour. Upon completion, the resulting mixture was concentrated under reduced pressure. The crude residue was directly purified by silica gel column chromatography, eluted with petroleum ether/ethyl acetate (1:1) to afford methyl 3,3-dimethyl-2-(pyridin-2-ylformamido)butanoate (20 g, 58%) as a yellow oil. LCMS (ESI) [M+H]⁺: 251.3.

### Step 2: methyl 3,3-dimethyl-1-picolinoylazetidine-2-carboxylate

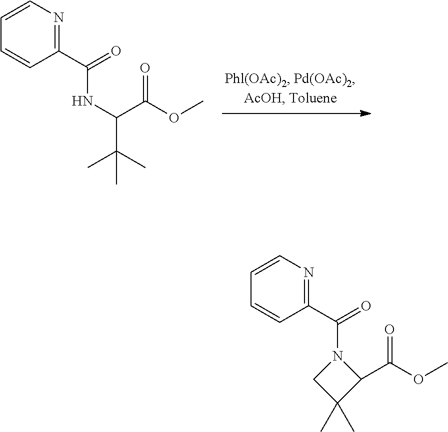 [SMILES: COC(=O)C(NC(=O)c1ccccn1)C(C)(C)C ; COC(=O)C1N(C(=O)c2ccccn2)CC1(C)C]

A solution of methyl 3,3-dimethyl-2-(pyridin-2-ylformamido)butanoate (5.0 g, 20.0 mmol), Pd(OAc)₂ (224.2 mg, 1.0 mmol) and PhI(OAc)₂ (16.18 g, 49.9 mmol) in acetic acid (1 mL) and toluene (50 mL) was stirred at 110° C. for 1 hour under an atmosphere of nitrogen. Upon completion, the resulting mixture was concentrated under reduced pressure. The crude residue was directly purified by silica gel column chromatography, eluted with petroleum ether/ethyl acetate (1:1) to afford methyl 3,3-dimethyl-1-(pyridine-2-carbonyl)azetidine-2-carboxylate (4.0 g, 80%) as a yellow oil. LCMS (ESI) [M+H]⁺: 249.2.

### Step 3: 3,3-dimethyl-1-picolinoylazetidine-2-carboxylic acid

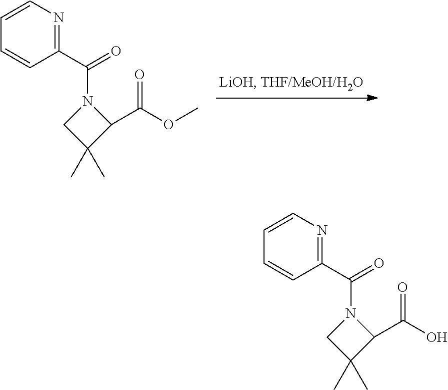 [SMILES: COC(=O)C1N(C(=O)c2ccccn2)CC1(C)C ; CC1(C)CN(C(=O)c2ccccn2)C1C(=O)O]

To a solution of methyl 3,3-dimethyl-1-(pyridine-2-carbonyl)azetidine-2-carboxylate (4.0 g, 16.1 mmol) in MeOH (20 mL) and THF (20 mL) was added lithium hydroxide monohydrate (6.76 g, 161 mmol) in H₂O (10 mL) at room temperature. The resulting mixture was stirred at 80° C. for 1 day. Upon completion, the resulting mixture was concentrated under reduced pressure. The crude residue was directly purified by reversed phase flash chromatography (conditions: column: C18 silica gel; mobile phase A: Water (0.1% FA), mobile phase B: MeCN; Gradient (B %): 0 to 100% in 25 min; detector: UV 254 nm) to afford 3,3-dimethyl-1-picolinoylazetidine-2-carboxylic acid (3.20 g, 85%) as a white solid. LCMS (ESI) [M+H]⁺: 130.1.

### Step 4: methyl 5-(4-aminophenyl)-6-methylpicolinate

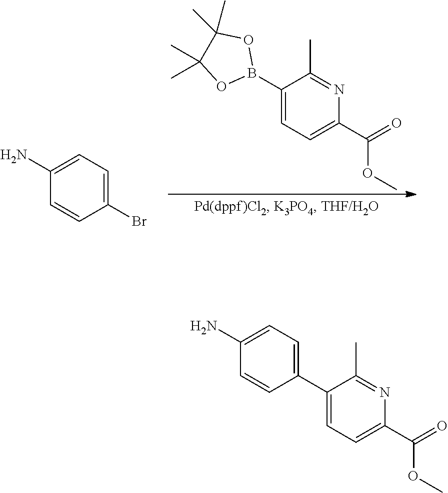

To a mixture of methyl 6-methyl-5-(4,4,5,5-tetramethyl-1,3,2-dioxaborolan-2-yl)picolinate (2.63 g, 9.50 mmol), 4-bromoaniline (1.63 g, 9.50 mmol) and Pd(dppf)Cl₂ (695 mg, 0.950 mmol) in THF (50 mL) was added a solution of K₃PO₄ (5.04 g, 23.8 mol) in H₂O (10 mL). The mixture was stirred at 60° C. for 2 hours under an atmosphere of nitrogen. Upon completion, the resulting mixture was concentrated under reduced pressure. The resulting mixture was diluted with water (50 mL) and extracted with ethyl acetate (2×50 mL). The combined organic layers were washed with H₂O (2×30 mL), dried over anhydrous Na₂SO₄, filtered and concentrated under reduced pressure. The crude residue was purified by silica gel column chromatography, eluted with petroleum ether/ethyl acetate (8:1) to afford methyl 5-(4-aminophenyl)-6-methylpicolinate (1.43 g, 32%) as a white solid. LCMS (ESI) [M+H]⁺: 243.

### Step 5: methyl 5-(4-(3,3-dimethyl-1-picolinoylazetidine-2-carboxamido)phenyl)-6-methylpicolinate

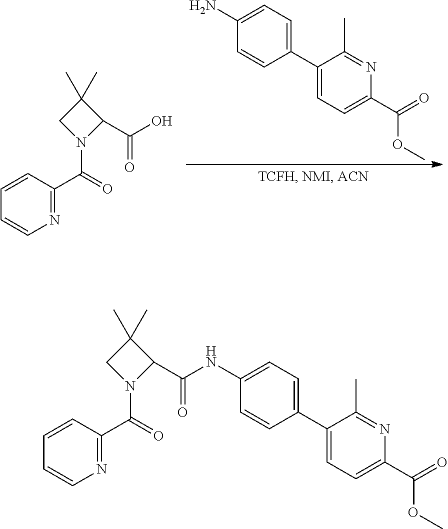 [SMILES: CC1(C)CN(C(=O)c2ccccn2)C1C(=O)O ; COC(=O)c1ccc(-c2ccc(N)cc2)c(C)n1 ; COC(=O)c1ccc(-c2ccc(NC(=O)C3N(C(=O)c4ccccn4)CC3(C)C)cc2)c(C)n1]

A solution of methyl 5-(4-aminophenyl)-6-methylpyridine-2-carboxylate (1.21 g, 5.0 mmol), TCFH (2.72 g, 10.0 mmol), NMI (2.6 g, 30.0 mmol) and 3,3-dimethyl-1-picolinoylazetidine-2-carboxylic acid (1.17 g, 5 mmol) in ACN (80 mL) was stirred at room temperature for 1 hour. Upon completion, the resulting mixture was concentrated under reduced pressure. The crude residue was directly purified by silica gel column chromatography, eluted with petroleum ether/ethyl acetate (1:1) to afford methyl 5-(4-(3,3-dimethyl-1-picolinoylazetidine-2-carboxamido)phenyl)-6-methylpicolinate (1.2 g, 52%) as a yellow solid. LCMS (ESI) [M+H]⁺: 459.2.

### Step 6: 5-(4-(3,3-dimethylazetidine-2-carboxamido)phenyl)-6-methylpicolinic acid

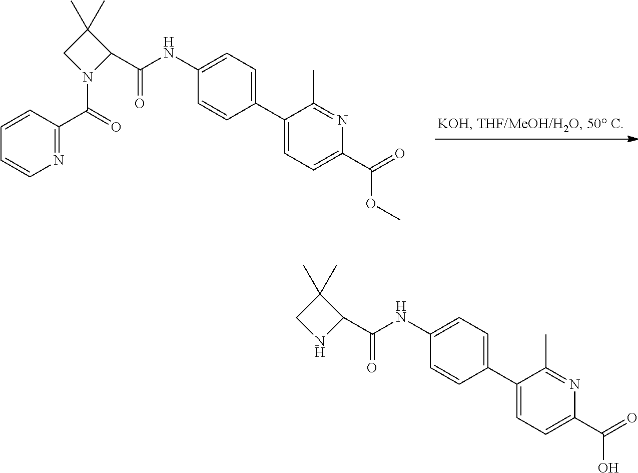 [SMILES: COC(=O)c1ccc(-c2ccc(NC(=O)C3N(C(=O)c4ccccn4)CC3(C)C)cc2)c(C)n1 ; Cc1nc(C(=O)O)ccc1-c1ccc(NC(=O)C2NCC2(C)C)cc1]

To a solution of methyl 5-(4-(3,3-dimethyl-1-picolinoylazetidine-2-carboxamido)phenyl)-6-methylpicolinate (1.2 g, 2.62 mmol) in MeOH (20 mL) and THF (20 mL) was added lithium hydroxide monohydrate (550 mg, 13.1 mmol) in H₂O (10 mL) at room temperature. The resulting mixture was stirred at 50° C. for 1 hour. Upon completion, the mixture was acidified to pH 6 with HCl (1M aq.). The resulting mixture was concentrated under reduced pressure. The crude residue was directly purified by reversed phase flash chromatography (conditions: column: C18 silica gel; mobile phase A: Water (0.1% FA), mobile phase B: MeCN; Gradient (B %): 0 to 100% in 25 min; detector: UV 254 nm) to afford 5-(4-(3,3-dimethylazetidine-2-carboxamido)phenyl)-6-methylpicolinic acid (750 mg, 84%) as a white solid. LCMS (ESI) [M+H]⁺: 340.1.

### Preparation of 4′-(3,3-dimethylazetidine-2-carboxamido)-[1,1′-biphenyl]-4-carboxylic acid

### Step 1: methyl 4′-amino-[1,1′-biphenyl]-4-carboxylate

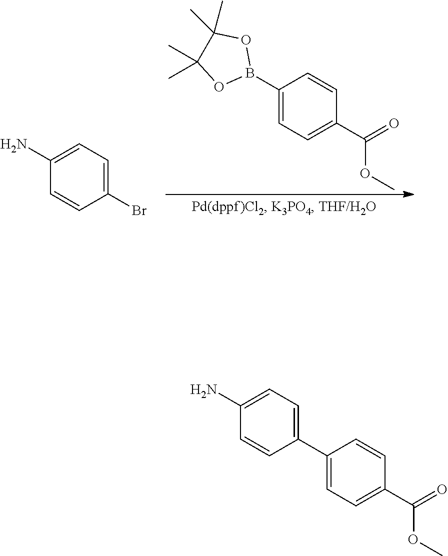

To a mixture of methyl 4-(4,4,5,5-tetramethyl-1,3,2-dioxaborolan-2-yl)benzoate (2.49 g, 9.50 mmol), 4-bromoaniline (1.63 g, 9.50 mmol) and Pd(dppf)Cl₂ (695 mg, 0.950 mmol) in THF (20 mL) was added solution of K₃PO₄ (5.04 g, 23.8 mol) in H₂O (10 mL) under nitrogen atmosphere. The mixture was stirred at 60° C. for 2 hours. Upon completion, the resulting mixture was concentrated under reduced pressure. The crude residue was purified by silica gel column chromatography, eluted with petroleum ether/ethyl acetate (8:1) to afford methyl 4′-amino-[1,1′-biphenyl]-4-carboxylate (1.68 g, 78%) as a white solid. LCMS (ESI) [M+H]⁺: 228.

### Step 2: methyl 4′-(3,3-dimethyl-1-picolinoylazetidine-2-carboxamido)-[1,1′-biphenyl]-4-carboxylate

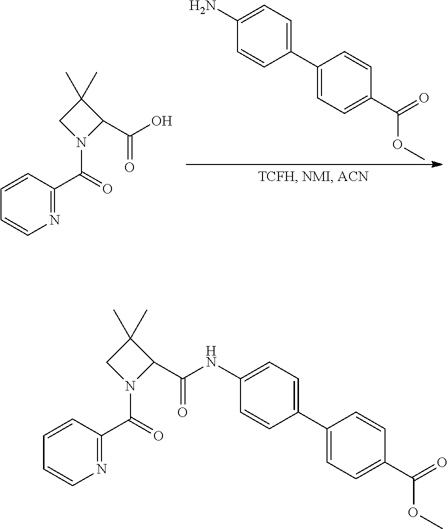 [SMILES: CC1(C)CN(C(=O)c2ccccn2)C1C(=O)O ; COC(=O)c1ccc(-c2ccc(N)cc2)cc1 ; COC(=O)c1ccc(-c2ccc(NC(=O)C3N(C(=O)c4ccccn4)CC3(C)C)cc2)cc1]

A solution of methyl 4′-amino-[1,1′-biphenyl]-4-carboxylate (1.14 g, 5.0 mmol), TCFH (2.72 g, 10.0 mmol), NMI (2.6 g, 30.0 mmol) and 3,3-dimethyl-1-picolinoylazetidine-2-carboxylic acid (1.17 g, 5 mmol) in ACN (30 mL) was stirred at room temperature for 1 hour. Upon completion, the resulting mixture was concentrated under reduced pressure. The crude residue was directly purified by silica gel column chromatography, eluted with petroleum ether/ethyl acetate (1:1) to afford methyl 4′-(3,3-dimethyl-1-picolinoylazetidine-2-carboxamido)-[1,1′-biphenyl]-4-carboxylate (1.37 g, 50%) as a yellow solid. LCMS (ESI) [M+H]⁺: 444.1.

### Step 3: 4′-(3,3-dimethylazetidine-2-carboxamido)-[1,1′-biphenyl]-4-carboxylic acid

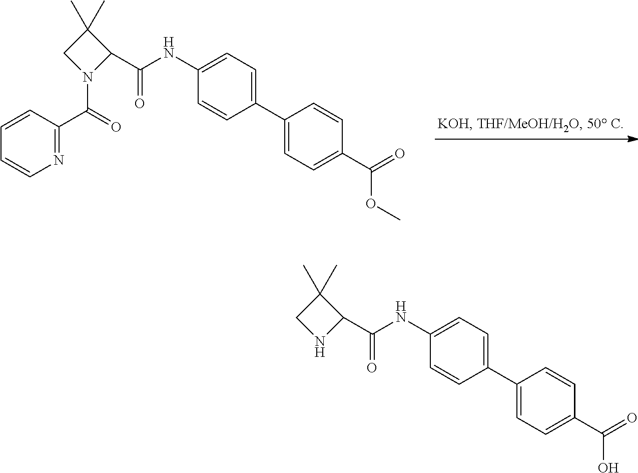 [SMILES: COC(=O)c1ccc(-c2ccc(NC(=O)C3N(C(=O)c4ccccn4)CC3(C)C)cc2)cc1 ; CC1(C)CNC1C(=O)Nc1ccc(-c2ccc(C(=O)O)cc2)cc1]

To a solution of methyl 4′-(3,3-dimethyl-1-picolinoylazetidine-2-carboxamido)-[1,1′-biphenyl]-4-carboxylate (1.37 g, 3.09 mmol) in MeOH (20 mL) and THF (20 mL) was added lithium hydroxide monohydrate (651 mg, 15.5 mmol) in H₂O (10 mL) at room temperature. The resulting mixture was stirred at 50° C. for 1 hour. Upon completion, the mixture was acidified to pH 6 with HCl (1M aq.) and then concentrated under reduced pressure. The crude residue was directly purified by reversed phase flash chromatography (conditions: column: C18 silica gel; mobile phase A: Water (0.1% FA), mobile phase B: MeCN; Gradient (B %): 0 to 100% in 25 min; detector: UV 254 nm) to afford 4′-(3,3-dimethylazetidine-2-carboxamido)-[1,1′-biphenyl]-4-carboxylic acid (785 mg, 78%) as a white solid. LCMS (ESI) [M+H]⁺: 325.1.

### Preparation of tert-butyl 4-(6-chloropyridin-3-yl)benzoate

### Step 1: tert-butyl 4-(6-chloropyridin-3-yl)benzoate

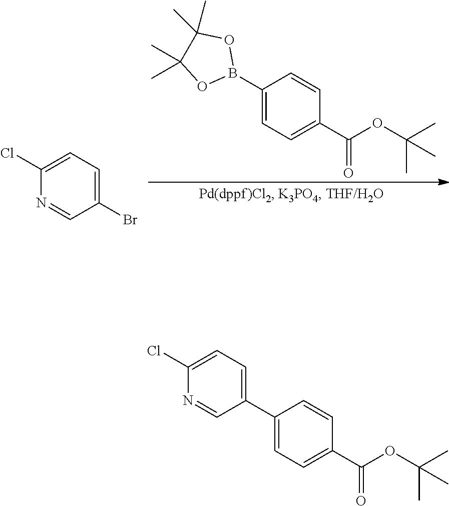 [SMILES: Clc1ccc(Br)cn1 ; CC(C)(C)OC(=O)c1ccc(B2OC(C)(C)C(C)(C)O2)cc1 ; CC(C)(C)OC(=O)c1ccc(-c2ccc(Cl)nc2)cc1]

To a mixture of tert-butyl 4-(4,4,5,5-tetramethyl-1,3,2-dioxaborolan-2-yl)benzoate (28.9 g, 95.0 mmol), 5-bromo-2-chloropyridine (18.3 g, 95.0 mmol) and Pd(dppf)Cl₂ (6.95 g, 9.50 mmol) in THF (200 mL) was added solution of K₃PO₄ (50.4 g, 2.38 mol) in H₂O (40 mL) under nitrogen atmosphere. The mixture was stirred at 60° C. for 2 hours. Upon completion, the resulting mixture was concentrated under reduced pressure. The crude residue was directly purified by silica gel column chromatography, eluted with petroleum ether/ethyl acetate (8:1) to afford tert-butyl 4-(6-chloropyridin-3-yl)benzoate (16.9 g, 61%) as a white solid. LCMS (ESI) [M+H]⁺: 290.

### Preparation of tert-butyl 4-(6-chloropyridin-3-yl)benzoate

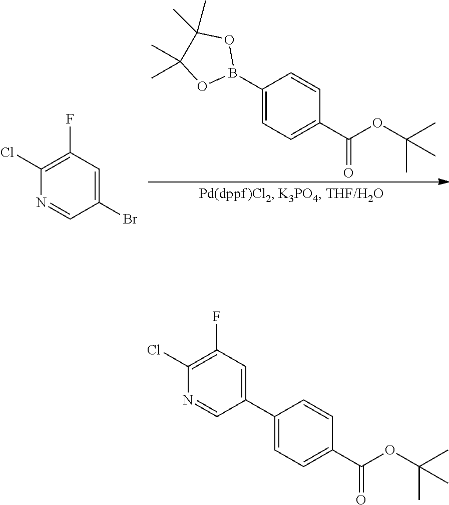 [SMILES: Fc1cc(Br)cnc1Cl ; CC(C)(C)OC(=O)c1ccc(B2OC(C)(C)C(C)(C)O2)cc1 ; CC(C)(C)OC(=O)c1ccc(-c2cnc(Cl)c(F)c2)cc1]

To a mixture of methyl tert-butyl 4-(4,4,5,5-tetramethyl-1,3,2-dioxaborolan-2-yl)benzoate (28.9 g, 95.0 mmol), 5-bromo-2-chloro-3-fluoropyridine (20.0 g, 95.0 mmol) and Pd(dppf)Cl₂ (6.95 g, 9.50 mmol) in THF (200 mL) was added a solution of K₃PO₄ (50.4 g, 238 mol) in H₂O (40 mL) under nitrogen atmosphere. The mixture was stirred at 60° C. for 2 hours. Upon completion, the resulting mixture was concentrated under reduced pressure. The crude residue was directly purified by silica gel column chromatography, eluted with petroleum ether/ethyl acetate (8:1) to afford tert-butyl 4-(6-chloropyridin-3-yl)benzoate (18.5 g, 63%) as a white solid. LCMS (ESI) [M+H]⁺: 308.

### Example 1: (S)-4-(6-(1-((4-isopropylphenyl)carbamoyl)pyrrolidin-2-yl)pyridin-3-yl)benzoic acid (Compound 1a) and (R)-4-(6-(1-((4-isopropylphenyl)carbamoyl)pyrrolidin-2-yl)pyridin-3-yl)benzoic acid (Compound 1b)

### Step 1: 2-(5-bromopyridin-2-yl)-N-(4-isopropylphenyl)pyrrolidine-1-carboxamide

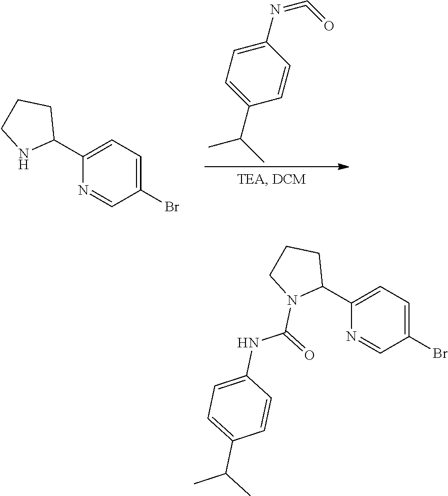 [SMILES: Brc1ccc(C2CCCN2)nc1 ; CC(C)c1ccc(N=C=O)cc1 ; CC(C)c1ccc(NC(=O)N2CCCC2c2ccc(Br)cn2)cc1]

A solution of 5-bromo-2-(pyrrolidin-2-yl)pyridine (1.0 g, 4.40 mmol), 1-isocyanato-4-isopropylbenzene (1.42 g, 8.81 mmol) and TEA (1.34 g, 13.21 mmol) in DCM (10 mL) were stirred at 25° C. for 1 hour. Upon completion, the resulting mixture was concentrated under reduced pressure. The crude residue was purified by silica gel column chromatography, eluted with PE/EA (1/2), to afford 2-(5-bromopyridin-2-yl)-N-(4-isopropylphenyl)pyrrolidine-1-carboxamide (800 mg, 47% yield) as a yellow solid. LCMS (ESI) [M+H]⁺: 388.0/390.0

### Step 2: methyl 4-(6-(1-((4-isopropylphenyl)carbamoyl)pyrrolidin-2-yl)pyridin-3-yl)benzoate

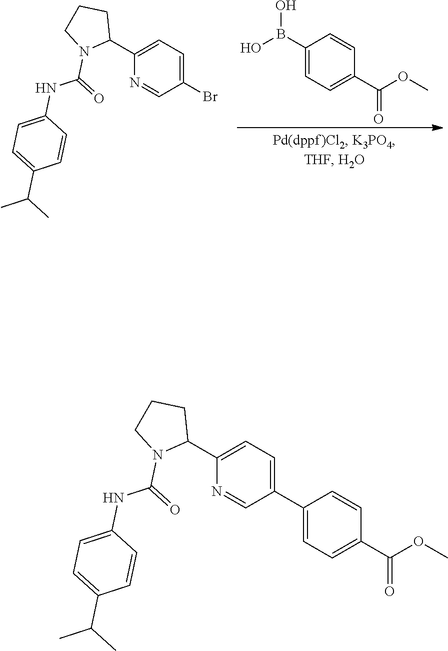 [SMILES: CC(C)c1ccc(NC(=O)N2CCCC2c2ccc(Br)cn2)cc1 ; COC(=O)c1ccc(B(O)O)cc1 ; COC(=O)c1ccc(-c2ccc(C3CCCN3C(=O)Nc3ccc(C(C)C)cc3)nc2)cc1]

A solution of 2-(5-bromopyridin-2-yl)-N-(4-isopropylphenyl)pyrrolidine-1-carboxamide (200 mg, 0.52 mmol), 4-(methoxycarbonyl)phenylboronic acid (92.7 mg, 0.52 mmol), Pd(dppf)Cl₂ (37.7 mg, 0.052 mmol) and K₃PO₄ (328 mg, 1.55 mmol) in THF (2 mL) and H₂O (0.2 mL) were stirred at 80° C. for 1 hour under nitrogen atmosphere. Upon completion, the resulting mixture was concentrated under reduced pressure. The crude residue was purified by silica gel column chromatography, eluted with PE/EA (1/1), to afford methyl 4-(6-(1-((4-isopropylphenyl)carbamoyl)pyrrolidin-2-yl)pyridin-3-yl)benzoate (150 mg, 66% yield) as a yellow oil. LCMS (ESI) [M+H]⁺: 444.2

### Step 3: 4-(6-(1-((4-isopropylphenyl)carbamoyl)pyrrolidin-2-yl)pyridin-3-yl)benzoic acid

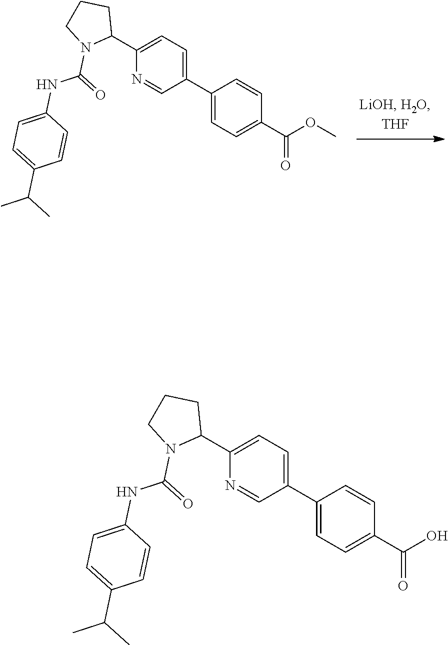

A solution of methyl 4-(6-(1-((4-isopropylphenyl)carbamoyl)pyrrolidin-2-yl)pyridin-3-yl)benzoate (80 mg, 0.180 mmol) and LiOH (8.6 mg, 0.36 mmol) in THF (1 mL) and H₂O (1 mL) was stirred at 25° C. for 1 hour. Upon completion, the mixture was acidified to pH=2 with HCl (1 M in water). The resulting mixture was extracted with EtOAc (3×10 mL). The combined organic layers were washed with H₂O (10 mL), dried over anhydrous Na₂SO₄, filtered and concentrated under reduced pressure. The crude residue was purified by reversed-phase flash chromatography (conditions: column, C18 silica gel; mobile phase, FA (0.1%) in H₂O/ACN, 10% to 60% gradient in 10 min; detector, UV 254 nm) to yield 4-(6-(1-((4-isopropylphenyl)carbamoyl)pyrrolidin-2-yl)pyridin-3-yl)benzoic acid (50 mg, 64% yield) as a white solid. LCMS (ESI) [M+H]⁺: 430.2

### Step 4: (S)-4-(6-(1-((4-isopropylphenyl)carbamoyl)pyrrolidin-2-yl)pyridin-3-yl)benzoic acid (Compound 1a) and (R)-4-(6-(1-((4-isopropylphenyl)carbamoyl)pyrrolidin-2-yl)pyridin-3-yl)benzoic acid (Compound 1b)

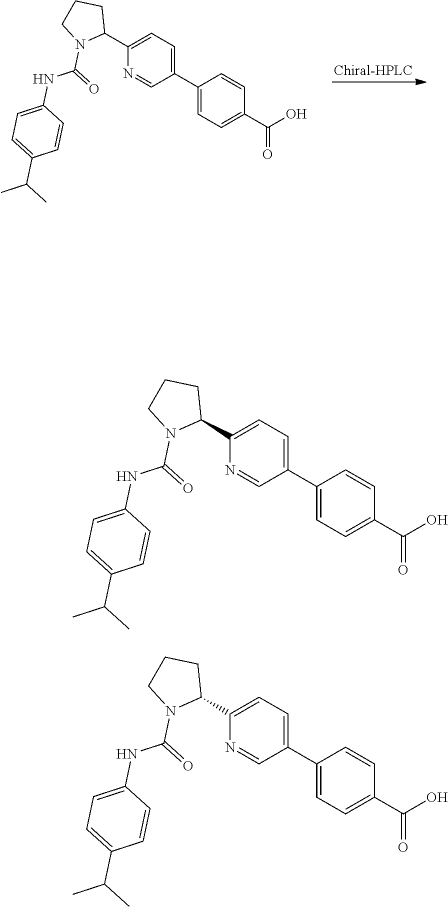

4-(6-(1-((4-isopropylphenyl)carbamoyl)pyrrolidin-2-yl)pyridin-3-yl)benzoic acid (40 mg, 0.093 mmol) was separated by prep-Chiral-HPLC (Column: CHIRAL ART Cellulose-SC, 3*25 cm, 5 μm; Mobile Phase A: Hex (0.1% FA), Mobile Phase B: EtOH; Flow rate: 40 mL/min; Gradient (B %): isocratic 30% B; Wave Length: 206/246 nm; RT1 (min): 7.61; RT2 (min): 15.22) to afford (S)-4-(6-(1-((4-isopropylphenyl)carbamoyl)pyrrolidin-2-yl)pyridin-3-yl)benzoic acid (Compound 1a) (1^{st} peak, 13.8 mg, 34% yield; Method A, Peak 1, 1.24 min) and (R)-4-(6-(1-((4-isopropylphenyl)carbamoyl)pyrrolidin-2-yl)pyridin-3-yl)benzoic acid (Compound 1b) (2^{nd} peak, 13.2 mg, 33% yield; Method A, Peak 2, 2.47 min).

| Cmpd# | Characterization Data |
|---|---|
| 1a | LCMS (ESI) [M + H]⁺: 430.2 |
| | ¹H NMR (400 MHz, Methanol-d₄) δ 8.81 (d, J = 2.1 Hz, 1H), 8.19-8.05 (m, 3H), 7.77 (d, J = 8.3 |
| | Hz, 2H), 7.46 (d, J = 8.2 Hz, 1H), 7.26 (d, J = 8.5 Hz, 2H), 7.10 (d, J = 8.5 Hz, 2H), 5.21 (dd, J = 8.0, |
| | 3.0 Hz, 1H), 3.93-3.82 (m, 1H), 3.76-3.61 (m, 1H), 2.83 (hept, J = 6.9 Hz, 1H), 2.52-2.37 (m, |
| | 1H), 2.16-2.00 (m, 3H), 1.20 (d, J = 6.9 Hz, 6H). |
| 1b | LCMS (ESI) [M + H]⁺: 430.2 |
| | ¹H NMR (400 MHz, Methanol-d₄) δ 8.81 (d, J = 2.0 Hz, 1H), 8.21-8.05 (m, 3H), 7.77 (d, J = 8.4 |
| | Hz, 2H), 7.46 (d, J = 8.2 Hz, 1H), 7.26 (d, J = 8.5 Hz, 2H), 7.10 (d, J = 8.5 Hz, 2H), 5.21 (dd, J = 7.9, |
| | 2.9 Hz, 1H), 3.92-3.81 (m, 1H), 3.77-3.64 (m, 1H), 2.83 (hept, J = 6.9 Hz, 1H), 2.52-2.37 (m, |
| | 1H), 2.16-1.98 (m, 3H), 1.20 (d, J = 6.9 Hz, 6H). |

### Example 2: (S)-2-(4-(6-(1-((4-isopropylphenyl)carbamoyl)pyrrolidin-2-yl)pyridin-3-yl)phenyl)acetic acid (Compound 2a) and (R)-2-(4-(6-(1-((4-isopropylphenyl)carbamoyl)pyrrolidin-2-yl)pyridin-3-yl)phenyl)acetic acid (Compound 2b)

### Step 1: methyl 2-(4-(6-(1-((4-isopropylphenyl)carbamoyl)pyrrolidin-2-yl)pyridin-3-yl)phenyl)acetate

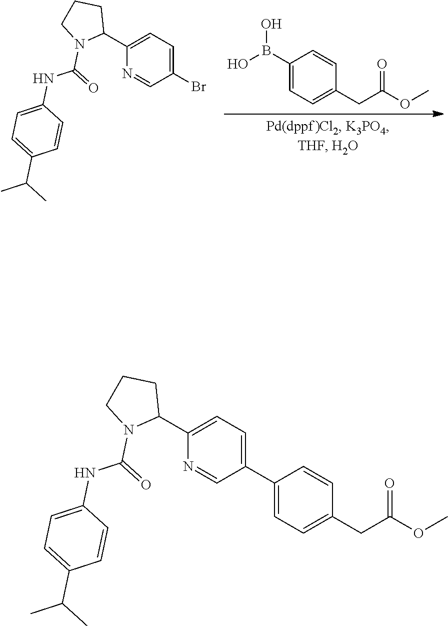 [SMILES: CC(C)c1ccc(NC(=O)N2CCCC2c2ccc(Br)cn2)cc1 ; COC(=O)Cc1ccc(B(O)O)cc1 ; COC(=O)Cc1ccc(-c2ccc(C3CCCN3C(=O)Nc3ccc(C(C)C)cc3)nc2)cc1]

A solution of 2-(5-bromopyridin-2-yl)-N-(4-isopropylphenyl)pyrrolidine-1-carboxamide (200 mg, 0.52 mmol), 4-(2-methoxy-2-oxoethyl)phenylboronic acid (99.9 mg, 0.52 mmol), Pd(dppf)Cl₂ (37.7 mg, 0.052 mmol) and K₃PO₄ (328 mg, 1.55 mmol) in THF (2 mL) H₂O (0.2 mL) was stirred at 80° C. for 1 hour under nitrogen atmosphere. Upon completion, the resulting mixture was concentrated under reduced pressure. The crude residue was purified by silica gel column chromatography, eluted with PE/EA (1/1), to afford methyl 2-(4-(6-(1-((4-isopropylphenyl)carbamoyl)pyrrolidin-2-yl)pyridin-3-yl)phenyl)acetate (150 mg, 63% yield) as a yellow oil. LCMS (ESI) [M+H]⁺: 458.2.

### Step 2: 2-(4-(6-(1-((4-isopropylphenyl)carbamoyl)pyrrolidin-2-yl)pyridin-3-yl)phenyl)acetic acid

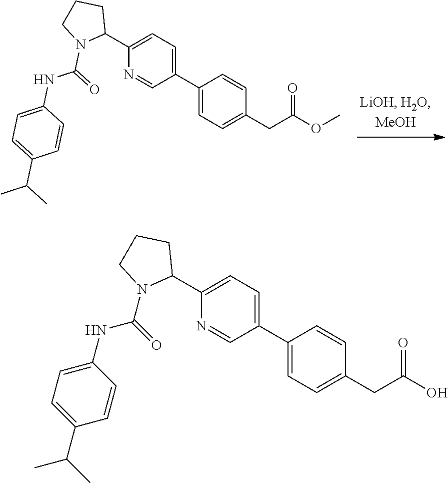

A solution of methyl 2-(4-(6-(1-((4-isopropylphenyl)carbamoyl)pyrrolidin-2-yl)pyridin-3-yl)phenyl)acetate (100 mg, 0.22 mmol) and LiOH (10.5 mg, 0.44 mmol) in THF (1 mL) and H₂O (1 mL) was stirred at 25° C. for 1 hour. Upon completion, the mixture was acidified to pH=2 with HCl (1 M in water). The resulting mixture was extracted with EtOAc (3×10 mL). The combined organic layers were washed with H₂O (10 mL), dried over anhydrous Na₂SO₄, filtered and concentrated under reduced pressure. The crude residue was purified by reversed-phase flash chromatography (conditions: column, C18 silica gel; mobile phase, FA (0.1%) in H₂O/ACN, 10% to 60% gradient in 10 min; detector, UV 254 nm) to yield 2-(4-(6-(1-((4-isopropylphenyl)carbamoyl)pyrrolidin-2-yl)pyridin-3-yl)phenyl)acetic acid (50 mg, 51% yield) as a white solid. LCMS (ESI) [M+H]⁺: 444.2.

### Step 3: (S)-2-(4-(6-(1-((4-isopropylphenyl)carbamoyl)pyrrolidin-2-yl)pyridin-3-yl)phenyl)acetic acid and (R)-2-(4-(6-(1-((4-isopropylphenyl)carbamoyl)pyrrolidin-2-yl)pyridin-3-yl)phenyl)acetic acid

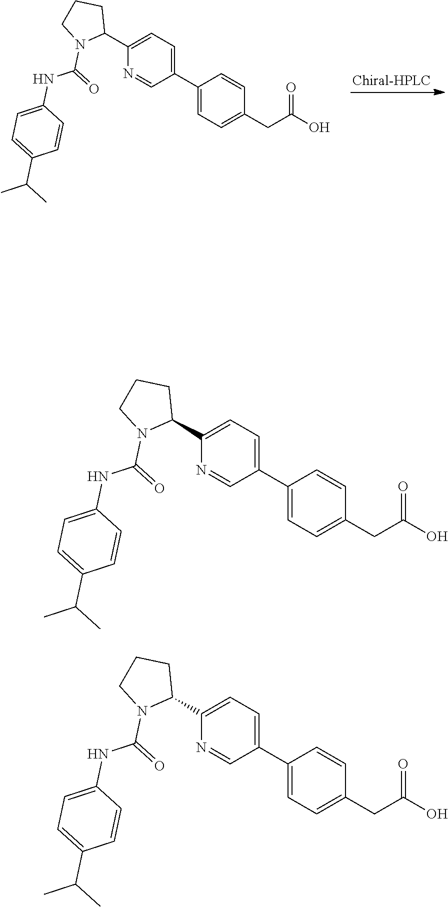

2-(4-(6-(1-((4-isopropylphenyl)carbamoyl)pyrrolidin-2-yl)pyridin-3-yl)phenyl)acetic acid (40 mg, 0.090 mmol) was separated by prep-Chiral-HPLC (Column: CHIRAL ART Cellulose-SC, 3*25 cm, 5 μm; Mobile Phase A: Hex (0.1% FA), Mobile Phase B: EtOH; Flow rate: 40 mL/min; Gradient (B %): isocratic 30% B; Wave Length: 206/246 nm; RT1 (min): 7.61; RT2 (min): 15.22) to afford (S)-2-(4-(6-(1-((4-isopropylphenyl)carbamoyl)pyrrolidin-2-yl)pyridin-3-yl)phenyl)acetic acid (Compound 2a) (1^{st} peak, 16.4 mg, 42% yield; Method B, Peak 1, 0.59 min) and (R)-2-(4-(6-(1-((4-isopropylphenyl)carbamoyl)pyrrolidin-2-yl)pyridin-3-yl)phenyl)acetic acid(Compound 2b) (2^{nd} peak, 14.7 mg, 38% yield; Method B, Peak 2, 0.94 min).

| Cmpd # | Characterization Data |
|---|---|
| 2a | LCMS (ESI) [M + H]⁺: 444.2 |
| | ¹H NMR (400 MHz, Methanol-d₄) δ 8.73 (d, J = 2.1 Hz, 1H), 8.03 (d, J = 9.8 Hz, 1H), 7.60 (d, |
| | J = 8.1 Hz, 2H), 7.42 (dd, J = 8.1, 5.0 Hz, 3H), 7.26 (d, J = 8.5 Hz, 2H), 7.10 (d, J = 8.5 Hz, 2H), |
| | 5.19 (dd, J = 7.9, 2.9 Hz, 1H), 3.88-3.81 (m, 1H), 3.74-3.61 (m, 3H), 2.82 (hept, J = 6.9 Hz, |
| | 1H), 2.54-2.37 (m, 1H), 2.13-2.00 (m, 3H), 1.19 (d, J = 6.9 Hz, 6H). |
| 2b | LCMS (ESI) [M + H]⁺: 444.2 |
| | ¹H NMR (400 MHz, Methanol-d₄) δ 8.73 (d, J = 1.9 Hz, 1H), 8.03 (d, J = 10.1 Hz, 1H), 7.60 (d, |
| | J = 8.1 Hz, 2H), 7.42 (dd, J = 8.0, 5.0 Hz, 3H), 7.26 (d, J = 8.5 Hz, 2H), 7.10 (d, J = 8.5 Hz, 2H), |
| | 5.19 (dd, J = 7.9, 2.8 Hz, 1H), 3.89-3.82 (m, 1H), 3.74-3.61 (m, 3H), 2.82 (hept, J = 6.9 Hz, |
| | 1H), 2.55-2.38 (m, 1H), 2.13-2.00 (m, 3H), 1.19 (d, J = 6.9 Hz, 6H). |

### Example 6: (R)-4′-(2-((4-isopropylphenyl)carbamoyl)pyrrolidine-1-carboxamido)-[1,1′-biphenyl]-4-carboxylic acid

### Step 1: tert-butyl (R)-2-((4-isopropylphenyl)carbamoyl)pyrrolidine-1-carboxylate

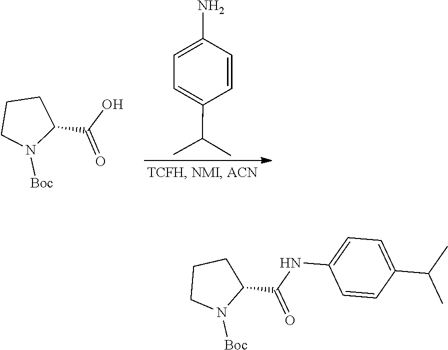

To a mixture of (tert-butoxycarbonyl)-D-proline (1.00 g, 4.6 mmol) and 4-isopropylaniline (691 mg, 5.1 mmol) in ACN (12 mL) were added N-(chloro(dimethylamino)methylene)-N-methylmethanaminium hexafluorophosphate(V) (1.56 g, 5.6 mmol) and 1-methyl-1H-imidazole (1.14 g, 13.9 mmol). The resulting mixture was stirred at room temperature for 1 hour. Upon completion, the mixture was concentrated under reduced pressure. The crude residue was purified by reversed-phase flash chromatography (conditions: column, C18 silica gel; mobile phase A: Water (10 mmol/L NH₄HCO₃); Mobile Phase B: MeCN; Gradient: 0% to 100% B in 20 min; detector, UV 254 nm) to give tert-butyl (R)-2-((4-isopropylphenyl)carbamoyl)pyrrolidine-1-carboxylate (1.1 g, 71% yield) as a light yellow solid. LCMS (ESI) [M+H]⁺: 333

### Step 2: (R)—N-(4-isopropylphenyl)pyrrolidine-2-carboxamide

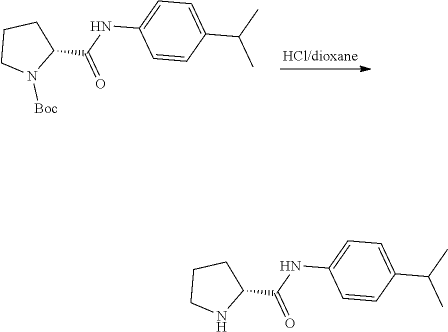 [SMILES: CC(C)c1ccc(NC(=O)[C@H]2CCCN2C(=O)OC(C)(C)C)cc1 ; CC(C)c1ccc(NC(=O)[C@H]2CCCN2)cc1]

To a mixture of tert-butyl (R)-2-((4-isopropylphenyl)carbamoyl)pyrrolidine-1-carboxylate (1.05 g, 3.2 mmol) in dioxane (2 mL) was added HCl (40 mmol, 10 mL, 4 M in dioxane) dropwise at 0° C. The resulting mixture was warmed to room temperature and stirred for 2 hours. Upon completion, the mixture was concentrated under reduced pressure to give (R)—N-(4-isopropylphenyl)pyrrolidine-2-carboxamide (820 mg, crude) as a yellow solid, which was used in the next step without further purification. LCMS (ESI) [M+H]⁺: 233

### Step 3: methyl (R)-4′-(2-((4-isopropylphenyl)carbamoyl)pyrrolidine-1-carboxamido)-[1,1′-biphenyl]-4-carboxylate

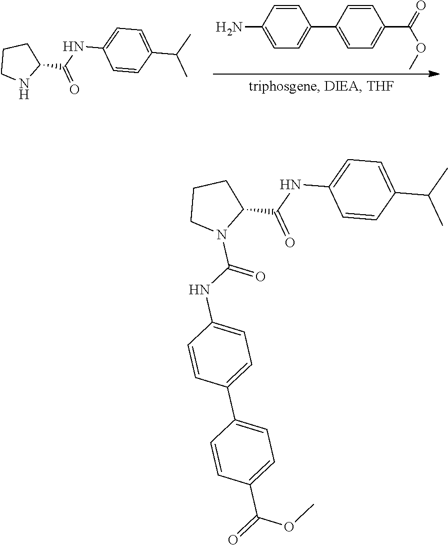 [SMILES: CC(C)c1ccc(NC(=O)[C@H]2CCCN2)cc1 ; COC(=O)c1ccc(-c2ccc(N)cc2)cc1 ; COC(=O)c1ccc(-c2ccc(NC(=O)N3CCC[C@@H]3C(=O)Nc3ccc(C(C)C)cc3)cc2)cc1]

To a solution of methyl 4′-amino-[1,1′-biphenyl]-4-carboxylate (147 mg, 0.6 mmol) in THF (5 mL) was added DIEA (417 mg, 3.2 mmol) and triphosgene (77 mg, 0.3 mmol) at 0° C. The resulting mixture was warmed to room temperature and stirred for 30 minutes. The reaction was cooled to 0° C. and (R)—N-(4-isopropylphenyl)pyrrolidine-2-carboxamide (150 mg, 0.6 mmol) was added. The mixture was warmed to room temperature and stirred for an additional 2 hours. Upon completion, the resulting mixture was diluted with water (8 mL) and extracted with EtOAc (3×10 mL). The combined organic layers were dried over anhydrous Na₂SO₄, filtered and concentrated under reduced pressure. The crude residue was purified by silica gel column chromatography, eluted with PE/EA (2:3) to afford methyl (R)-4′-(2-((4-isopropylphenyl)carbamoyl)pyrrolidine-1-carboxamido)-[1,1′-biphenyl]-4-carboxylate (180 mg, 57% yield) as a yellow solid. LCMS (ESI) [M+H]⁺: 486

### Step 4: (R)-4′-(2-((4-isopropylphenyl)carbamoyl)pyrrolidine-1-carboxamido)-[1,1′-biphenyl]-4-carboxylic acid

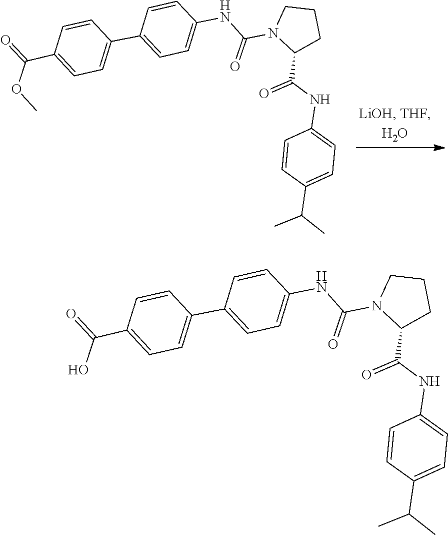 [SMILES: COC(=O)c1ccc(-c2ccc(NC(=O)N3CCC[C@@H]3C(=O)Nc3ccc(C(C)C)cc3)cc2)cc1 ; CC(C)c1ccc(NC(=O)[C@H]2CCCN2C(=O)Nc2ccc(-c3ccc(C(=O)O)cc3)cc2)cc1]

To a solution of methyl (R)-4′-(2-((4-isopropylphenyl)carbamoyl)pyrrolidine-1-carboxamido)-[1,1′-biphenyl]-4-carboxylate (100 mg, 0.2 mmol) in THF (1.5 mL) was added a solution of LiOH monohydrate (26 mg, 0.6 mmol) and H₂O (1.5 mL). The resulting mixture was stirred at room temperature for 2 hours. Upon completion, the mixture was acidified to pH 3 with HCl (1 M) and then concentrated under reduced pressure. The crude residue was purified by Prep-HPLC (conditions: Column: Xselect CSH Prep C18, 30*150 mm 5 μm; Mobile Phase A: Water (0.1% FA), Mobile Phase B: ACN; Flow rate: 60 mL/min; Gradient (B %): 36% to 61% B in 10 min; Wave Length: 254 nm/220 nm; RT1 (min): 11.37) to afford (R)-4′-(2-((4-isopropylphenyl)carbamoyl)pyrrolidine-1-carboxamido)-[1,1′-biphenyl]-4-carboxylic acid (44.0 mg Compound 6) as an off-white solid.

| Cmpd | |
|---|---|
| # | Characterization Data |
| 6 | LCMS (ESI) [M + H]⁺: 472.3 |
| | ¹H NMR (400 MHz, DMSO-d₆) δ 12.89 (bs, 1H), 9.91 |
| | (s, 1H), 8.44 (s, 1H), 7.97 (d, J = 8.0 Hz, 2H), 7.76 (d, J = |
| | 8.4 Hz, 2H), 7.70-7.60 (m, 4H), 7.51 (d, J = 8.4 Hz, 2H), 7.16 |
| | (d, J = 8.4 Hz, 2H), 4.49-4.46 (m, 1H), 3.70-3.64 (m, 1H), 3.58- |
| | 3.49 (m, 1H), 2.86-2.79 (m, 1H), 2.19-2.16 (m, 1H), 2.06-1.90 |
| | (m, 3H), 1.17 (d, J = 7.2 Hz, 6H). |

### Example 7: (S)-3-fluoro-4′-(1-((4-isopropylphenyl)carbamoyl)piperidine-2-carboxamido)-[1,1′-biphenyl]-4-carboxylic acid

### Step 1: methyl 4′-((tert-butoxycarbonyl)amino)-3-fluoro-[1,1′-biphenyl]-4-carboxylate

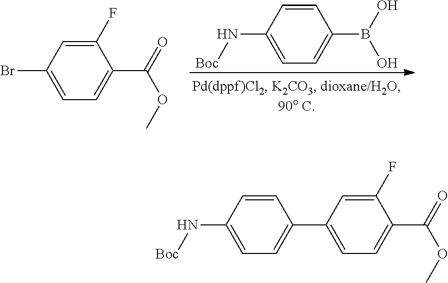 [SMILES: COC(=O)c1ccc(Br)cc1F ; CC(C)(C)OC(=O)Nc1ccc(B(O)O)cc1 ; COC(=O)c1ccc(-c2ccc(NC(=O)OC(C)(C)C)cc2)cc1F]

To a mixture of methyl 4-bromo-2-fluorobenzoate (20.0 g, 85.8 mmol) and (4-((tert-butoxycarbonyl)amino)phenyl)boronic acid (30.5 g, 129 mmol) in dioxane (300 mL) were added H₂O (75 mL), K₂CO₃ (35.6 g, 258 mmol) and Pd(dppf)Cl₂·CH₂Cl₂ (7.01 g, 8.5 mmol) at room temperature under nitrogen atmosphere. The resulting mixture was heated at 90° C. for 1 hour. Upon completion, the mixture was allowed to cool down to room temperature and was then filtered. The filter cake was washed with DCM (3×350 mL). The combined filtrates were concentrated under reduced pressure. The crude residue was purified by silica gel column chromatography, eluted with PE/EA (3:1) to afford methyl 4′-((tert-butoxycarbonyl)amino)-3-fluoro-[1,1′-biphenyl]-4-carboxylate (23.0 g, 78% yield) as a yellow solid. LCMS (ESI) [M+H]⁺: 346

### Step 2: methyl 4′-amino-3-fluoro-[1,1′-biphenyl]-4-carboxylate

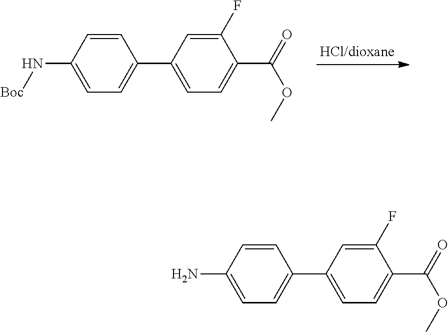 [SMILES: COC(=O)c1ccc(-c2ccc(NC(=O)OC(C)(C)C)cc2)cc1F ; COC(=O)c1ccc(-c2ccc(N)cc2)cc1F]

To a mixture of methyl 4′-((tert-butoxycarbonyl)amino)-3-fluoro-[1,1′-biphenyl]-4-carboxylate (10.0 g, 29.0 mmol) in dioxane (30 mL) was added HCl (600 mmol, 150 mL, 4 M in dioxane) dropwise at 0° C. The resulting mixture was warmed to room temperature and stirred for 1 hour. Upon completion, the reaction mixture was concentrated under reduced pressure to give methyl 4′-amino-3-fluoro-[1,1′-biphenyl]-4-carboxylate (7.00 g, crude) as a white solid, which was used in the next step without further purification. LCMS (ESI) [M+H]⁺: 246

### Step 3: tert-butyl (S)-2-((3′-fluoro-4′-(methoxycarbonyl)-[1,1′-biphenyl]-4-yl)carbamoyl)piperidine-1-carboxylate

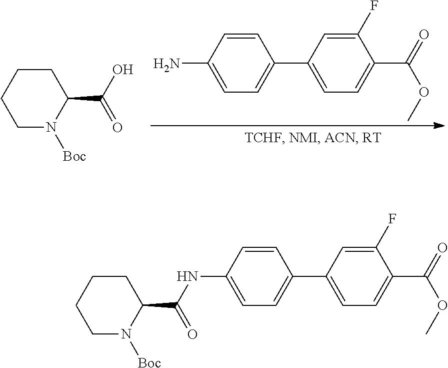 [SMILES: CC(C)(C)OC(=O)N1CCCC[C@H]1C(=O)O ; COC(=O)c1ccc(-c2ccc(N)cc2)cc1F ; COC(=O)c1ccc(-c2ccc(NC(=O)[C@@H]3CCCCN3C(=O)OC(C)(C)C)cc2)cc1F]

A solution of (S)-1-(tert-butoxycarbonyl)piperidine-2-carboxylic acid (200 mg, 0.9 mmol), methyl 4′-amino-3-fluoro-[1,1′-biphenyl]-4-carboxylate (214 mg, 0.9 mmol), N-(chloro(dimethylamino)methylene)-N-methylmethanaminium hexafluorophosphate(V) (293 mg, 1.0 mmol) and 1-methyl-1H-imidazole (215 mg, 2.6 mmol) in ACN (6 mL) was stirred for 2 hours at room temperature. Upon completion, the reaction mixture was directly purified by reverse phase flash chromatography (conditions: column, C18 silica gel; mobile phase A, Water (10 mmol/L NH₄HCO₃), mobile phase B: MeCN; Gradient (B %): 0% to 100% B in 20 min; detector, UV 254 nm) to yield tert-butyl (S)-2-((3′-fluoro-4′-(methoxycarbonyl)-[1,1′-biphenyl]-4-yl)carbamoyl)piperidine-1-carboxylate (160 mg, 40% yield) as a white solid. LCMS (ESI) [M+H]⁺: 457

### Step 4: methyl (S)-3-fluoro-4′-(piperidine-2-carboxamido)-[1,1′-biphenyl]-4-carboxylate

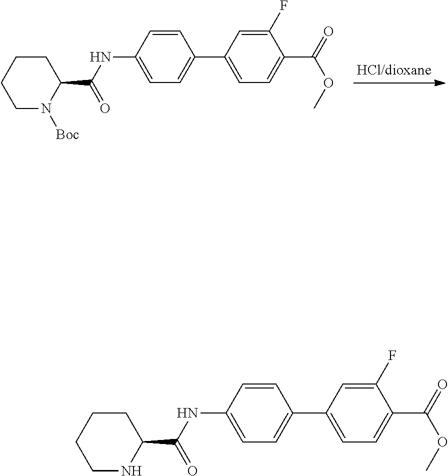 [SMILES: COC(=O)c1ccc(-c2ccc(NC(=O)[C@@H]3CCCCN3C(=O)OC(C)(C)C)cc2)cc1F ; COC(=O)c1ccc(-c2ccc(NC(=O)[C@@H]3CCCCN3)cc2)cc1F]

To a solution of tert-butyl (S)-2-((3′-fluoro-4′-(methoxycarbonyl)-[1,1′-biphenyl]-4-yl)carbamoyl)piperidine-1-carboxylate (140 mg, 0.3 mmol) in dioxane (1 mL) was added HCl (20 mmol, 5 mL, 4 M in dioxane) dropwise at 0° C. The resulting mixture was warmed to room temperature and stirred for 2 hours. Upon completion, the resulting mixture was concentrated under reduced pressure to afford methyl (S)-3-fluoro-4′-(piperidine-2-carboxamido)-[1,1′-biphenyl]-4-carboxylate (100 mg, crude) as a white solid, which was used in the next step without further purification. LCMS (ESI) [M+H]⁺: 357

### Step 5: methyl (S)-3-fluoro-4′-(1-((4-isopropylphenyl)carbamoyl)piperidine-2-carboxamido)-[1,1′-biphenyl]-4-carboxylate

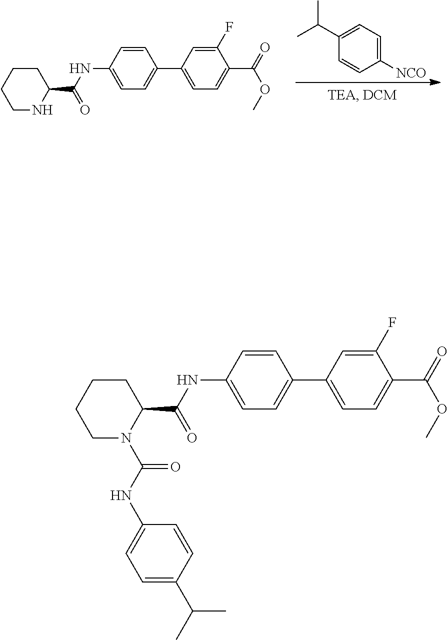

To a solution of methyl (S)-3-fluoro-4′-(piperidine-2-carboxamido)-[1,1′-biphenyl]-4-carboxylate (90 mg, 0.2 mmol) in DCM (3 mL) was added triethylamine (25 mg, 0.2 mmol) and 1-isocyanato-4-isopropylbenzene (41 mg, 0.2 mmol) sequentially at 0° C. The resulting mixture was warmed to room temperature and stirred for 2 hours. Upon completion, the reaction was quenched with ice water at 0° C. The resulting mixture was extracted with EtOAc (3×6 mL). The combined organic layers were dried over anhydrous Na₂SO₄, filtered and concentrated under reduced pressure. The crude residue was purified by reversed-phase flash chromatography (conditions: column, C18 silica gel; mobile phase A: water (10 mmol/L NH₄HCO₃), Mobile Phase B: MeCN; Gradient (B %): 0% to 100% B in 20 min; detector, UV 254 nm) to yield methyl (S)-3-fluoro-4′-(1-((4-isopropylphenyl)carbamoyl)piperidine-2-carboxamido)-[1,1′-biphenyl]-4-carboxylate (80 mg, 61% yield) as a white solid. LCMS (ESI) [M+H]⁺: 518

### Step 6: (S)-3-fluoro-4′-(1-((4-isopropylphenyl)carbamoyl)piperidine-2-carboxamido)-[1,1′-biphenyl]-4-carboxylic acid

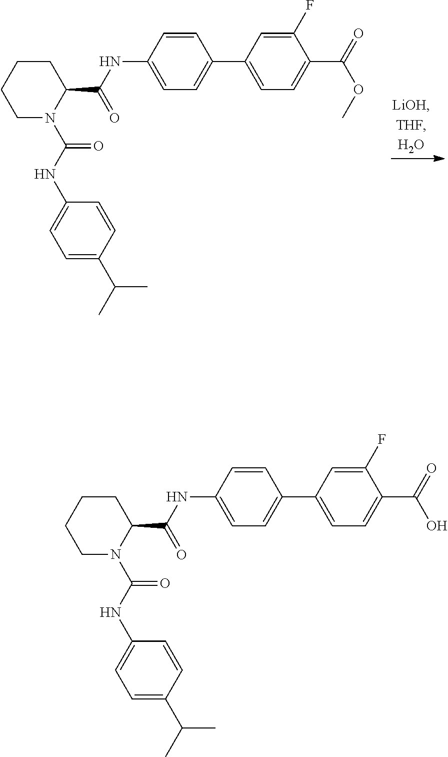 [SMILES: COC(=O)c1ccc(-c2ccc(NC(=O)[C@@H]3CCCCN3C(=O)Nc3ccc(C(C)C)cc3)cc2)cc1F ; CC(C)c1ccc(NC(=O)N2CCCC[C@H]2C(=O)Nc2ccc(-c3ccc(C(=O)O)c(F)c3)cc2)cc1]

To a solution of methyl (S)-3-fluoro-4′-(1-((4-isopropylphenyl)carbamoyl)piperidine-2-carboxamido)-[1,1′-biphenyl]-4-carboxylate (78 mg, 0.1 mmol) in THF (2 mL) was added a solution of LiOH monohydrate (11 mg, 0.4 mmol) in H₂O (2 mL) and at 0° C. The resulting mixture was warmed to room temperature and stirred for 2 hours. Upon completion, the mixture was acidified to pH 5 with aqueous HCl (2 M) at 0° C. and then was concentrated under reduced pressure. The crude residue was purified by Prep-HPLC (conditions: Column: Xselect CSH Prep C18, 30*150 mm 5 μm; Mobile Phase A: Water (0.1% formic acid), Mobile Phase B: MeOH; Flow rate: 60 mL/min; Gradient (B %): 57% to 75% B in 12 min; Wave Length: 254 nm/220 nm; RT1 (min): 13.55) to afford (S)-3-fluoro-4′-(1-((4-isopropylphenyl)carbamoyl)piperidine-2-carboxamido)-[1,1′-biphenyl]-4-carboxylic acid (26.3 mg, Compound 7; Method C, Peak 1, 2.11 min—analyzed against Compound 8) as a white solid.

| Cmpd | |
|---|---|
| # | Characterization Data |
| 7 | LCMS (ESI) [M + H]⁺: 504.3 |
| | ¹H NMR (400 MHz, DMSO-d₆) δ 13.17 (bs, 1H), 10.07 |
| | (s, 1H), 8.48 (s, 1H), 7.93-7.91 (m, 1H), 7.76 (s, 4H), 7.63-7.60 |
| | (m, 2H), 7.38-7.35 (m, 2H), 7.10 (d, J = 8.4 Hz, 2H), 4.91 (d, |
| | J = 5.6 Hz, 1H), 3.97 (d, J = 12.4 Hz, 1H), 3.44-3.41 (m, 1H), |
| | 2.84-2.81 (m, 1H), 2.18 (d, J = 13.6 Hz, 1H), 1.72-1.62 (m, |
| | 3H), 1.47-1.23 (m, 2H), 1.13 (d, J = 6.8 Hz, 6H). |

### Example 8: (R)-3-fluoro-4′-(1-((4-isopropylphenyl)carbamoyl)piperidine-2-carboxamido)-[1,1′-biphenyl]-4-carboxylic acid

### Step 1: tert-butyl (R)-2-((3′-fluoro-4′-(methoxycarbonyl)-[1,1′-biphenyl]-4-yl)carbamoyl)piperidine-1-carboxylate

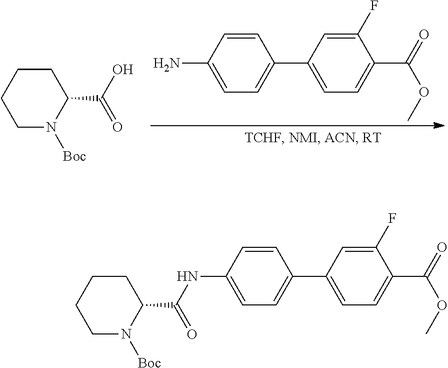 [SMILES: CC(C)(C)OC(=O)N1CCCC[C@@H]1C(=O)O ; COC(=O)c1ccc(-c2ccc(N)cc2)cc1F ; COC(=O)c1ccc(-c2ccc(NC(=O)[C@H]3CCCCN3C(=O)OC(C)(C)C)cc2)cc1F]

A solution of (R)-1-(tert-butoxycarbonyl)piperidine-2-carboxylic acid (200 mg, 0.8 mmol), methyl 4′-amino-3-fluoro-[1,1′-biphenyl]-4-carboxylate (224 mg, 0.9 mmol), N-(chloro(dimethylamino)methylene)-N-methylmethanaminium hexafluorophosphate(V) (256 mg, 0.9 mmol) and 1-methyl-1H-imidazole (358 mg, 4.3 mmol) in ACN (6 mL) was stirred for 2 hours at room temperature. Upon completion, the mixture was concentrated under reduced pressure. The crude residue was purified by reversed-phase flash chromatography (conditions: column, C18 silica gel; mobile phase A: Water (10 mmol/L NH₄HCO₃), Mobile Phase B: MeCN; Gradient (B %): 0% to 100% B in 20 min; detector, UV 254 nm) to give tert-butyl (R)-2-((3′-fluoro-4′-(methoxycarbonyl)-[1,1′-biphenyl]-4-yl)carbamoyl)piperidine-1-carboxylate (180 mg, 45% yield) as a white solid. LCMS (ESI) [M+H]⁺: 457

### Step 2: methyl (R)-3-fluoro-4′-(piperidine-2-carboxamido)-[1,1′-biphenyl]-4-carboxylate

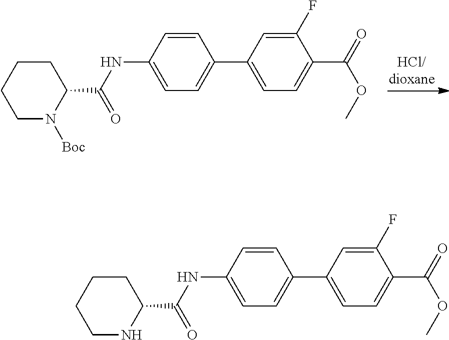 [SMILES: COC(=O)c1ccc(-c2ccc(NC(=O)[C@H]3CCCCN3C(=O)OC(C)(C)C)cc2)cc1F ; COC(=O)c1ccc(-c2ccc(NC(=O)[C@H]3CCCCN3)cc2)cc1F]

To a solution of tert-butyl (R)-2-((3′-fluoro-4′-(methoxycarbonyl)-[1,1′-biphenyl]-4-yl)carbamoyl)piperidine-1-carboxylate (160 mg, 0.3 mmol) in dioxane (1 mL) was added HCl (20 mmol, 5 mL, 4 M in dioxane) dropwise at 0° C. The resulting mixture was warmed to room temperature and stirred for 2 hours. Upon completion, the resulting mixture was concentrated under reduced pressure to afford methyl (R)-3-fluoro-4′-(piperidine-2-carboxamido)-[1,1′-biphenyl]-4-carboxylate (120 mg, crude) as a white solid, which was used in the next step without further purification. LCMS (ESI) [M+H]⁺: 357

### Step 3: methyl (R)-3-fluoro-4′-(1-((4-isopropylphenyl)carbamoyl)piperidine-2-carboxamido)-[1,1′-biphenyl]-4-carboxylate

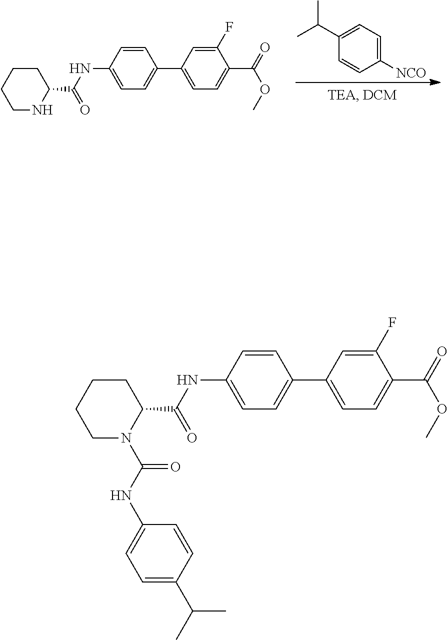

To a solution of methyl (R)-3-fluoro-4′-(piperidine-2-carboxamido)-[1,1′-biphenyl]-4-carboxylate (100 mg, 0.2 mmol) in DCM (3 mL) were added triethylamine (51 mg, 0.5 mmol) and 1-isocyanato-4-isopropylbenzene (41 mg, 0.2 mmol) at 0° C. under nitrogen atmosphere. The resulting mixture was warmed to room temperature and stirred for 2 hours. Upon completion, the reaction was quenched with ice water at 0° C. The resulting mixture was extracted with EtOAc (3×6 mL). The combined organic layers were dried over anhydrous Na₂SO₄, filtered and concentrated under reduced pressure. The crude residue was purified by reversed-phase flash chromatography (conditions: column, C18 silica gel; mobile phase A: Water (10 mmol/L NH₄HCO₃), Mobile Phase B: MeCN; Gradient (B %): 0% to 100% B in 20 min; detector, UV 254 nm) to give methyl (R)-3-fluoro-4′-(1-((4-isopropylphenyl)carbamoyl)piperidine-2-carboxamido)-[1,1′-biphenyl]-4-carboxylate (90 mg, 68% yield) as a white solid. LCMS (ESI) [M+H]⁺: 518

### Step 4: (R)-3-fluoro-4′-(1-((4-isopropylphenyl)carbamoyl)piperidine-2-carboxamido)-[1,1′-biphenyl]-4-carboxylic acid

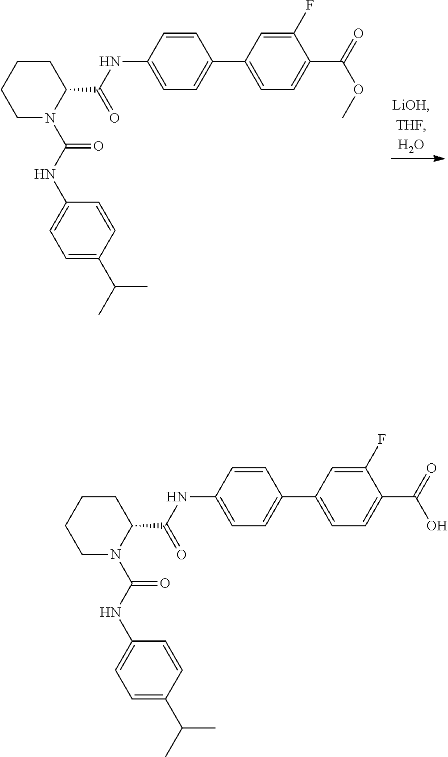 [SMILES: COC(=O)c1ccc(-c2ccc(NC(=O)[C@H]3CCCCN3C(=O)Nc3ccc(C(C)C)cc3)cc2)cc1F ; CC(C)c1ccc(NC(=O)N2CCCC[C@@H]2C(=O)Nc2ccc(-c3ccc(C(=O)O)c(F)c3)cc2)cc1]

To a solution of methyl (R)-3-fluoro-4′-(1-((4-isopropylphenyl)carbamoyl)piperidine-2-carboxamido)-[1,1′-biphenyl]-4-carboxylate (88 mg, 0.1 mmol) in THF (2 mL) was added a solution of LiOH monohydrate (4 mg, 0.1 mmol) in H₂O (2 mL) at 0° C. under nitrogen atmosphere. The resulting mixture was warmed to room temperature and stirred for 2 hours. Upon completion, the mixture was acidified to pH 5 with aqueous HCl (2 M) at 0° C. The resulting solution was concentrated under reduced pressure. The crude residue was purified by Prep-HPLC (conditions: Column: XSelect CSH Fluoro Phenyl 30*150 mm, 5 μm; Mobile Phase A: Water (0.1% FA), Mobile Phase B: MeCN; Flow rate: 60 mL/min; Gradient (B %): 45% B to 55% B in 15 min; Wave Length: 254 nm/220 nm; RT1 (min): 11.4) to afford (R)-3-fluoro-4′-(1-((4-isopropylphenyl)carbamoyl)piperidine-2-carboxamido)-[1,1′-biphenyl]-4-carboxylic acid (26.6 mg, Compound 8; Method C, Peak 2, 3.82 min—analyzed against Compound 7) as a white solid.

| Cmpd | |
|---|---|
| # | Characterization Data |
| 8 | LCMS (ESI) [M + H]⁺: 504.2 |
| | ¹H NMR (400 MHz, DMSO-d₆) δ 13.17 (bs, 1H), 10.07 |
| | (s, 1H), 8.48 (s, 1H), 7.92 (t, J = 8.2 Hz, 1H), 7.76 (s, 4H), |
| | 7.63-7.60 (m, 2H), 7.39 (d, J = 8.8 Hz, 2H), 7.10 (d, J = 8.4 Hz, |
| | 2H), 4.91 (d, J = 5.6 Hz, 1H), 3.97 (d, J = 12.5 Hz, 1H), 3.49- |
| | 3.38 (m, 1H), 2.89-2.72 (m, 1H), 2.18 (d, J = 13.6 Hz, 1H), |
| | 1.79-1.61 (m, 3H), 1.47-1.31 (m, 2H), 1.14 (d, J = 6.8 Hz, 6H). |

### Example 9: (R)-3-fluoro-4′-(1-((4-isopropylphenyl)carbamoyl)-2-methylazetidine-2-carboxamido)-[1,1′-biphenyl]-4-carboxylic acid

### Step 1: tert-butyl (R)-2-((3′-fluoro-4′-(methoxycarbonyl)-[1,1′-biphenyl]-4-yl)carbamoyl)-2-methylazetidine-1-carboxylate

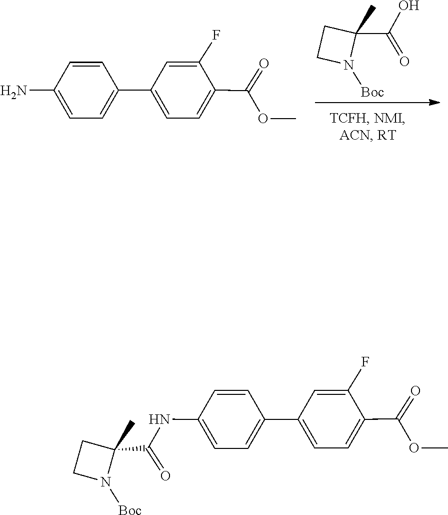 [SMILES: COC(=O)c1ccc(-c2ccc(N)cc2)cc1F ; CC(C)(C)OC(=O)N1CC[C@]1(C)C(=O)O ; COC(=O)c1ccc(-c2ccc(NC(=O)[C@@]3(C)CCN3C(=O)OC(C)(C)C)cc2)cc1F]

A solution of methyl 4′-amino-3-fluoro-[1,1′-biphenyl]-4-carboxylate (200 mg, 0.8 mmol), (R)-1-(tert-butoxycarbonyl)-2-methylazetidine-2-carboxylic acid (175 mg, 0.8 mmol), N-(chloro(dimethylamino)methylene)-N-methylmethanaminium hexafluorophosphate(V) (343 mg, 1.2 mmol) and 1-methyl-1H-imidazole (201 mg, 2.4 mmol) in ACN (6 mL) was stirred for 2 hours at room temperature. Upon completion, the resulting mixture was concentrated under reduced pressure. The crude residue was purified by reversed-phase flash chromatography (conditions: column, C18 silica gel; mobile phase A: Water (10 mmol/L NH₄HCO₃), Mobile Phase B: MeCN; Gradient (B %): 0% to 100% in 20 min; detector, UV 254 nm) to yield tert-butyl (R)-2-((3′-fluoro-4′-(methoxycarbonyl)-[1,1′-biphenyl]-4-yl)carbamoyl)-2-methylazetidine-1-carboxylate (310 mg, 85% yield) as a white solid. LCMS (ESI) [M+H]⁺: 443

### Step 2: methyl (R)-3-fluoro-4′-(2-methylazetidine-2-carboxamido)-[1,1′-biphenyl]-4-carboxylate

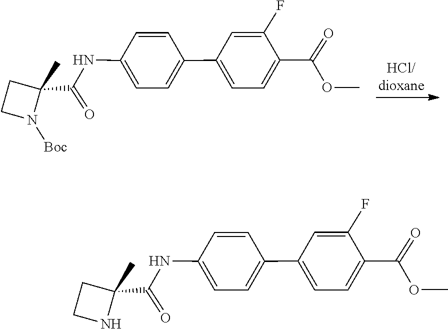 [SMILES: COC(=O)c1ccc(-c2ccc(NC(=O)[C@@]3(C)CCN3C(=O)OC(C)(C)C)cc2)cc1F ; COC(=O)c1ccc(-c2ccc(NC(=O)[C@@]3(C)CCN3)cc2)cc1F]

To a mixture of tert-butyl (R)-2-((3′-fluoro-4′-(methoxycarbonyl)-[1,1′-biphenyl]-4-yl)carbamoyl)-2-methylazetidine-1-carboxylate (200 mg, 0.4 mmol) in dioxane (1 mL) was added HCl (20 mmol, 5 mL, 4 M in dioxane) dropwise at 0° C. The resulting mixture was warmed to room temperature stirred for 2 hours. Upon completion, the resulting mixture was concentrated under reduced pressure to give methyl (R)-3-fluoro-4′-(2-methylazetidine-2-carboxamido)-[1,1′-biphenyl]-4-carboxylate (150 mg, crude) as a light yellow solid, which was used in the next step without further purification. LCMS (ESI) [M+H]⁺: 343

### Step 3: methyl (R)-3-fluoro-4′-(1-((4-isopropylphenyl)carbamoyl)-2-methylazetidine-2-carboxamido)-[1,1′-biphenyl]-4-carboxylate

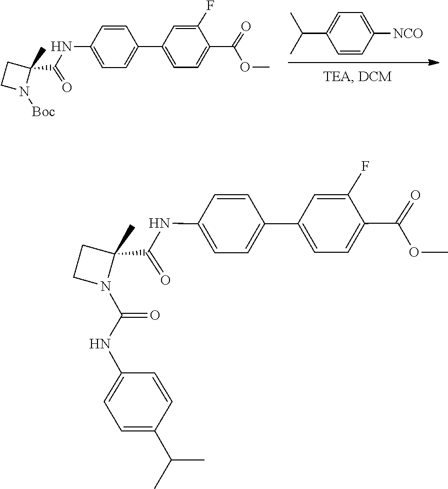

To a solution of methyl (R)-3-fluoro-4′-(2-methylazetidine-2-carboxamido)-[1,1′-biphenyl]-4-carboxylate (100 mg, 0.1 mmol) in DCM (3 mL) were added triethylamine (15 mg, 0.1 mmol) and 1-isocyanato-4-isopropylbenzene (47 mg, 0.1 mmol) at 0° C. The resulting mixture was stirred at room temperature for 2 hours. Upon completion, the reaction was quenched with ice water at 0° C. The resulting mixture was extracted with EtOAc (3×6 mL). The combined organic layers were dried over anhydrous Na₂SO₄, filtered and concentrated under reduced pressure. The crude residue was purified by silica gel column chromatography, eluted with PE/EA (1:1) to afford methyl (R)-3-fluoro-4′-(1-((4-isopropylphenyl)carbamoyl)-2-methylazetidine-2-carboxamido)-[1,1′-biphenyl]-4-carboxylate (140 mg, 95% yield) as a white solid. LCMS (ESI) [M+H]⁺: 504

### Step 4: (R)-3-fluoro-4′-(1-((4-isopropylphenyl)carbamoyl)-2-methylazetidine-2-carboxamido)-[1,1′-biphenyl]-4-carboxylic acid

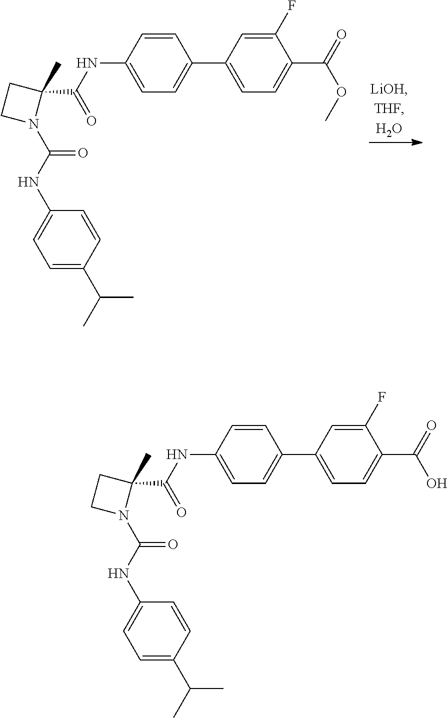 [SMILES: COC(=O)c1ccc(-c2ccc(NC(=O)[C@@]3(C)CCN3C(=O)Nc3ccc(C(C)C)cc3)cc2)cc1F ; CC(C)c1ccc(NC(=O)N2CC[C@]2(C)C(=O)Nc2ccc(-c3ccc(C(=O)O)c(F)c3)cc2)cc1]

To a solution of methyl (R)-3-fluoro-4′-(1-((4-isopropylphenyl)carbamoyl)-2-methylazetidine-2-carboxamido)-[1,1′-biphenyl]-4-carboxylate (120 mg, 0.3 mmol) in THF (3 mL) was added a solution of LiOH monohydrate (30 mg, 0.9 mmol) in H₂O (3 mL) at 0° C. The resulting mixture was warmed to room temperature and stirred for 2 hours. Upon completion, the mixture was acidified to pH 5 with aqueous HCl (2 M) at 0° C. and then was concentrated under reduced pressure. The crude residue was purified by Prep-HPLC (conditions: Column: Xselect CSH Prep C18, 30*150 mm 5 μm; Mobile Phase A: Water (0.1% FA), Mobile Phase B: ACN; Flow rate: 60 mL/min; Gradient (B %): 48% to 69% B in 10 min; Wave Length: 254 nm/220 nm; RT1(min): 10.52) to afford (R)-3-fluoro-4′-(1-((4-isopropylphenyl)carbamoyl)-2-methylazetidine-2-carboxamido)-[1,1′-biphenyl]-4-carboxylic acid (45.7 mg, Compound 9; Method D, Peak 1, 3.17 min—analyzed against Compound 10) as a white solid.

| Cmpd | |
|---|---|
| # | Characterization Data |
| 9 | LCMS (ESI) [M + H]⁺: 490.3 |
| | ¹H NMR (400 MHz, DMSO-d₆) δ 13.20 (bs, 1H), 10.74 |
| | (s, 1H), 8.58 (s, 1H), 7.94 (t, J = 8.0 Hz, 1H), 7.80 (d, J = 8.8 |
| | Hz, 2H), 7.71 (d, J = 8.8 Hz, 2H), 7.65-7.63 (m, 2H), 7.45 (d, |
| | J = 6.8 Hz, 2H), 7.16 (d, J = 8.4 Hz, 2H), 4.19-4.02 (m, 1H), |
| | 3.92-3.83 (m, 1H), 2.88-2.76 (m, 1H), 2.69-2.58 (m, 1H), 2.18- |
| | 2.03 (m, 1H), 1.72 (s, 3H), 1.17 (d, J = 8.4 Hz, 6H). |

### Example 10: (S)-3-fluoro-4′-(1-((4-isopropylphenyl)carbamoyl)-2-methylazetidine-2-carboxamido)-[1,1′-biphenyl]-4-carboxylic acid

### Step 1: tert-butyl (S)-2-((3′-fluoro-4′-(methoxycarbonyl)-[1,1′-biphenyl]-4-yl)carbamoyl)-2-methylazetidine-1-carboxylate

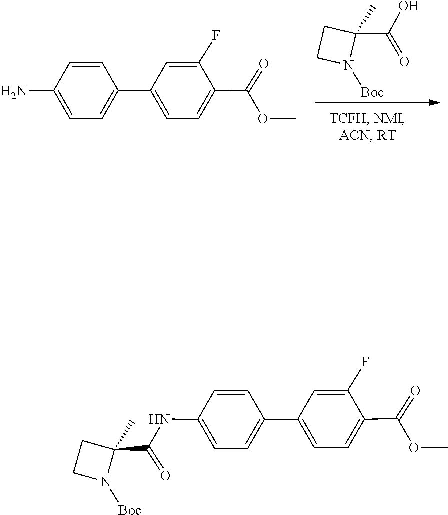 [SMILES: COC(=O)c1ccc(-c2ccc(N)cc2)cc1F ; CC(C)(C)OC(=O)N1CC[C@@]1(C)C(=O)O ; COC(=O)c1ccc(-c2ccc(NC(=O)[C@]3(C)CCN3C(=O)OC(C)(C)C)cc2)cc1F]

A solution of methyl 4′-amino-3-fluoro-[1,1′-biphenyl]-4-carboxylate (120 mg, 0.5 mmol), (S)-1-(tert-butoxycarbonyl)-2-methylazetidine-2-carboxylic acid (211 mg, 1.0 mmol), N-(chloro(dimethylamino)methylene)-N-methylmethanaminium hexafluorophosphate(V) (206 mg, 0.7 mmol) and 1-methyl-1H-imidazole (402 mg, 4.9 mmol) in ACN (6 mL) was stirred for 2 hours at room temperature. Upon completion, the resulting mixture was concentrated under reduced pressure. The crude residue was purified by reversed-phase flash chromatography (conditions: column, C18 silica gel; mobile phase A: Water (10 mmol/L NH₄HCO₃), Mobile Phase B: MeCN; Gradient (B %): 0% to 100% in 20 min; detector, UV 254 nm) to give tert-butyl (S)-2-((3′-fluoro-4′-(methoxycarbonyl)-[1,1′-biphenyl]-4-yl)carbamoyl)-2-methylazetidine-1-carboxylate (100 mg, 46% yield) as a yellow solid. LCMS (ESI) [M+H]⁺: 443

### Step 2: methyl (S)-3-fluoro-4′-(2-methylazetidine-2-carboxamido)-[1,1′-biphenyl]-4-carboxylate

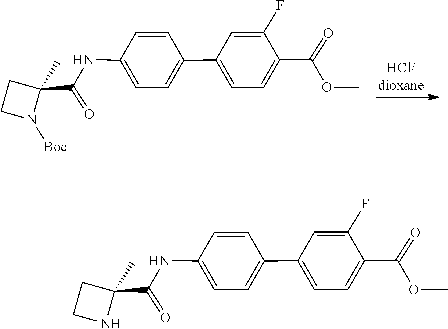 [SMILES: COC(=O)c1ccc(-c2ccc(NC(=O)[C@]3(C)CCN3C(=O)OC(C)(C)C)cc2)cc1F ; COC(=O)c1ccc(-c2ccc(NC(=O)[C@]3(C)CCN3)cc2)cc1F]

To a mixture of tert-butyl (S)-2-((3′-fluoro-4′-(methoxycarbonyl)-[1,1′-biphenyl]-4-yl)carbamoyl)-2-methylazetidine-1-carboxylate (100 mg, 0.2 mmol) in dioxane (0.6 mL) was added HCl (12 mmol, 3 mL, 4 M in dioxane) dropwise at 0° C. The resulting mixture was warmed to room temperature and stirred for 2 hours. Upon completion, the resulting mixture was concentrated under reduced pressure to give methyl (S)-3-fluoro-4′-(2-methylazetidine-2-carboxamido)-[1,1′-biphenyl]-4-carboxylate (90 mg, crude) as a light yellow solid, which was used in the next step without further purification. LCMS (ESI) [M+H]⁺: 343

### Step 3: methyl (S)-3-fluoro-4′-(1-((4-isopropylphenyl)carbamoyl)-2-methylazetidine-2-carboxamido)-[1,1′-biphenyl]-4-carboxylate

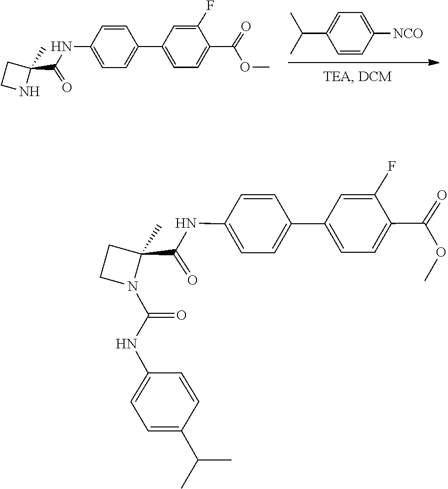

To a solution of methyl (S)-3-fluoro-4′-(2-methylazetidine-2-carboxamido)-[1,1′-biphenyl]-4-carboxylate (90 mg, 0.3 mmol) in DCM (3 mL) was added triethylamine (27 mg, 0.3 mmol) and 1-isocyanato-4-isopropylbenzene (42 mg, 0.3 mmol) at 0° C. The resulting mixture was warmed to room temperature and stirred for 2 hours. Upon completion, the reaction was quenched with ice water at 0° C. The resulting mixture was extracted with EtOAc (3×6 mL). The combined organic layers were dried over anhydrous Na₂SO₄, filtered and concentrated under reduced pressure. The crude residue was purified by reversed-phase flash chromatography (conditions: column, C18 silica gel; mobile phase A: water (0.1% formic acid), Mobile Phase B: MeCN; Gradient (B %): 0% to 100% in 20 min; detector, UV 254 nm) to give methyl (S)-3-fluoro-4′-(1-((4-isopropylphenyl)carbamoyl)-2-methylazetidine-2-carboxamido)-[1,1′-biphenyl]-4-carboxylate (100 mg, 76% yield) as white solid. LCMS (ESI) [M+H]⁺: 504

### Step 4: (S)-3-fluoro-4′-(1-((4-isopropylphenyl)carbamoyl)-2-methylazetidine-2-carboxamido)-[1,1′-biphenyl]-4-carboxylic acid

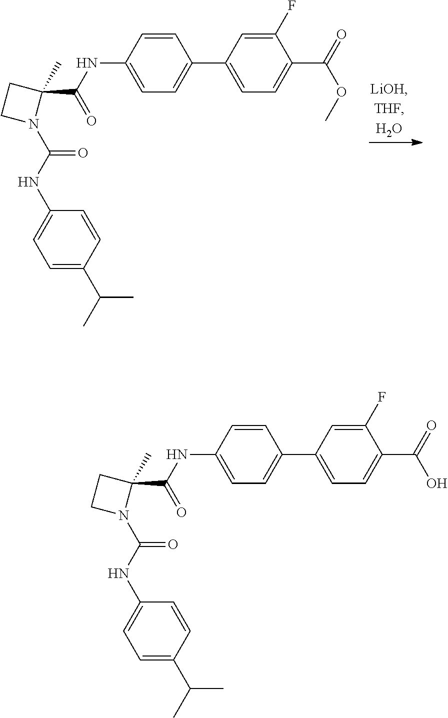 [SMILES: COC(=O)c1ccc(-c2ccc(NC(=O)[C@]3(C)CCN3C(=O)Nc3ccc(C(C)C)cc3)cc2)cc1F ; CC(C)c1ccc(NC(=O)N2CC[C@@]2(C)C(=O)Nc2ccc(-c3ccc(C(=O)O)c(F)c3)cc2)cc1]

To a solution of methyl (S)-3-fluoro-4′-(1-((4-isopropylphenyl)carbamoyl)-2-methylazetidine-2-carboxamido)-[1,1′-biphenyl]-4-carboxylate (88 mg, 0.2 mmol) in THF (3 mL) were added a solution of LiOH monohydrate (22 mg, 0.6 mmol) in H₂O (3 mL) at 0° C. under nitrogen atmosphere. The resulting mixture was warmed to room temperature and stirred for 2 hours. Upon completion, the mixture was acidified to pH 5 with aqueous HCl (2 M) at 0° C. and then was concentrated under reduced pressure. The crude residue was purified by Prep-HPLC (conditions (Column: Xbridge Phenyl OBD Column, 30*150 mm 5 μm; Mobile Phase A: Water (0.1% FA), Mobile Phase B: ACN; Flow rate: 60 mL/min; Gradient (B %): 39% B to 59% B in 15 min; Wave Length: 254 nm/220 nm; RT1 (min): 10.2) to afford (S)-3-fluoro-4′-(1-((4-isopropylphenyl)carbamoyl)-2-methylazetidine-2-carboxamido)-[1,1′-biphenyl]-4-carboxylic acid (41.4 mg, Compound 10; Method D, Peak 2, 3.51 min—analyzed against Compound 9) as a white solid.

| Cmpd | |
|---|---|
| # | Characterization Data |
| 10 | LCMS (ESI) [M + H]⁺: 490.2 |
| | ¹H NMR (400 MHz, DMSO-d₆) δ 13.24 (bs, 1H), 10.74 |
| | (s, 1H), 8.59 (s, 1H), 7.94 (t, J = 8.0 Hz, 1H), 7.80 (d, J = |
| | 8.8 Hz, 2H), 7.71 (d, J = 8.8 Hz, 2H), 7.64-7.63 (m, 2H), 7.45 |
| | (d, J = 6.8 Hz, 2H), 7.16 (d, J = 8.4 Hz, 2H), 4.19-4.02 (m, 1H), |
| | 3.92-3.83 (m, 1H), 2.88-2.76 (m, 1H), 2.69-2.58 (m, 1H), 2.18- |
| | 2.03 (m, 1H), 1.72 (s, 3H), 1.17 (d, J = 8.4 Hz, 6H). |

### Example 11: (S)-3-fluoro-4′-(1-((4-isopropylphenyl)carbamoyl)azepane-2-carboxamido)-[1,1′-biphenyl]-4-carboxylic acid

### Step 1: tert-butyl (S)-2-((4′-(tert-butoxycarbonyl)-3′-fluoro-[1,1′-biphenyl]-4-yl)carbamoyl)azepane-1-carboxylate

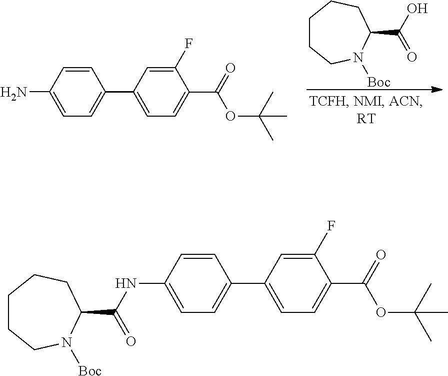 [SMILES: CC(C)(C)OC(=O)c1ccc(-c2ccc(N)cc2)cc1F ; CC(C)(C)OC(=O)N1CCCCC[C@H]1C(=O)O ; CC(C)(C)OC(=O)c1ccc(-c2ccc(NC(=O)[C@@H]3CCCCCN3C(=O)OC(C)(C)C)cc2)cc1F]

A solution of (S)-1-(tert-butoxycarbonyl)azepane-2-carboxylic acid (169 mg, 0.7 mmol), tert-butyl 4′-amino-3-fluoro-[1,1′-biphenyl]-4-carboxylate (200 mg, 0.7 mmol), N-(chloro(dimethylamino)methylene)-N-methylmethanaminium hexafluorophosphate(V) (293 mg, 1.0 mmol) and 1-methyl-1H-imidazole (171 mg, 2.1 mmol) in ACN (6 mL) was stirred for 2 hours at room temperature. Upon completion, the resulting mixture was concentrated under reduced pressure. The crude residue was purified by reversed-phase flash chromatography (conditions: column, C18 silica gel; mobile phase A: Water (10 mmol/L NH₄HCO₃), Mobile Phase B: MeCN; Gradient (B %): 5% to 95% in 25 min; detector, UV 254 nm) to yield tert-butyl (S)-2-((4′-(tert-butoxycarbonyl)-3′-fluoro-[1,1′-biphenyl]-4-yl)carbamoyl)azepane-1-carboxylate (180 mg, 62% yield) as a white solid. LCMS (ESI) [M+H]⁺: 513

### Step 2: (S)-4′-(azepane-2-carboxamido)-3-fluoro-[1,1′-biphenyl]-4-carboxylic acid

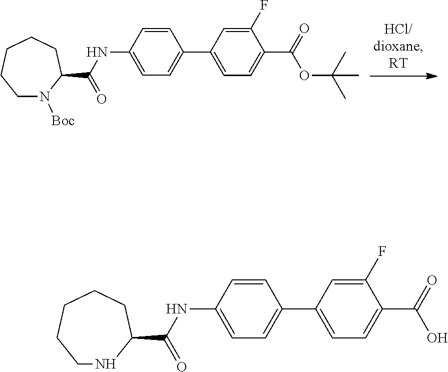 [SMILES: CC(C)(C)OC(=O)c1ccc(-c2ccc(NC(=O)[C@@H]3CCCCCN3C(=O)OC(C)(C)C)cc2)cc1F ; O=C(O)c1ccc(-c2ccc(NC(=O)[C@@H]3CCCCCN3)cc2)cc1F]

To a solution of tert-butyl (S)-2-((4′-(tert-butoxycarbonyl)-3′-fluoro-[1,1′-biphenyl]-4-yl)carbamoyl)azepane-1-carboxylate (200 mg, 0.4 mmol) in dioxane (1 mL) was added HCl in (20 mmol, 5 mL, 4 M in dioxane) dropwise at 0° C. The resulting mixture was warmed to room temperature and stirred for 2 hours. Upon completion, the resulting mixture was concentrated under reduced pressure to give (S)-4′-(azepane-2-carboxamido)-3-fluoro-[1,1′-biphenyl]-4-carboxylic acid (150 mg, crude) as a light yellow solid, which was used in the next step without further purification. LCMS (ESI) [M+H]⁺: 357

### Step 3: (S)-3-fluoro-4′-(1-((4-isopropylphenyl)carbamoyl)azepane-2-carboxamido)-[1,1′-biphenyl]-4-carboxylic acid

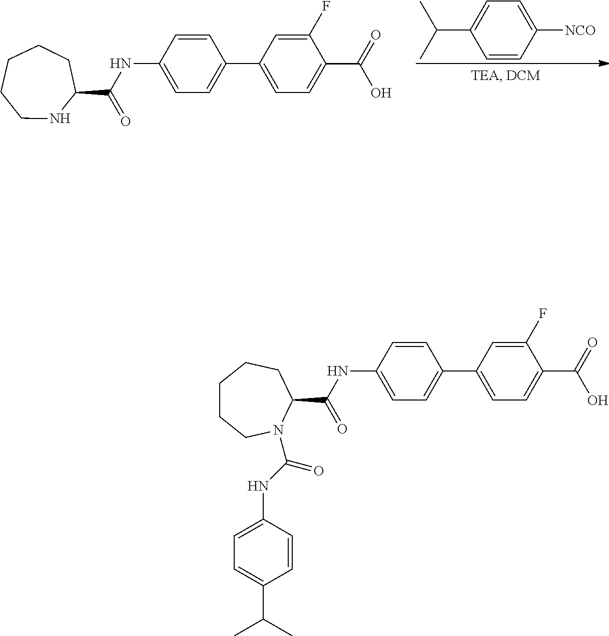

To a mixture of (S)-4′-(azepane-2-carboxamido)-3-fluoro-[1,1′-biphenyl]-4-carboxylic acid (100 mg, 0.2 mmol) in DCM (4 mL) were added triethylamine (28 mg, 0.2 mmol) and 1-isocyanato-4-isopropylbenzene (45 mg, 0.2 mmol) at 0° C. The resulting mixture was warmed to room temperature and stirred for 2 hours. Upon completion, the reaction was quenched with ice water at 0° C. The resulting mixture was extracted with EtOAc (3×6 mL). The combined organic layers were dried over anhydrous Na₂SO₄, filtered and concentrated under reduced pressure. The crude residue was purified by Prep-HPLC (conditions: Column: Xbridge Phenyl OBD Column, 30*150 mm 5 μm; Mobile Phase A: Water (0.1% FA), Mobile Phase B: MeCN; Flow rate: 60 mL/min; Gradient (B %): 41% B to 57% B in 15 min; Wave Length: 254 nm/220 nm; RT1(min): 12.2) to afford (S)-3-fluoro-4′-(1-((4-isopropylphenyl)carbamoyl)azepane-2-carboxamido)-[1,1′-biphenyl]-4-carboxylic acid (18.2 mg, Compound 11; Method C, Peak 1, 1.65 min—analyzed against Compound 12) as a white solid.

| Cmpd | |
|---|---|
| # | Characterization Data |
| 11 | LCMS (ESI) [M + H]⁺: 518.2 |
| | ¹H NMR (400 MHz, DMSO-d₆) δ 13.19 (bs, 1H), 10.10 |
| | (s, 1H), 8.26 (s, 1H), 7.93-7.91 (m, 1H), 7.77-7.76 (m, 4H), |
| | 7.63-7.60 (m, 2H), 7.39 (d, J = 8.4 Hz, 2H), 7.08 (d, J = 8.4 Hz, |
| | 2H), 4.81-4.72 (m, 1H), 3.97 (d, J = 15.2 Hz, 1H), 3.31-3.29 |
| | (m, 1H), 2.88-2.83 (m, 1H), 2.33-2.29 (m, 1H), 1.92-1.89 (m, |
| | 1H), 1.85-1.67 (m, 3H), 1.49-1.38 (m, 1H), 1.32-1.29 (m, 2H), |
| | 1.17 (d, J = 6.8 Hz, 6H). |

### Example 12: (R)-3-fluoro-4′-(1-((4-isopropylphenyl)carbamoyl)azepane-2-carboxamido)-[1,1′-biphenyl]-4-carboxylic acid

### Step 1: tert-butyl (R)-2-((4′-(tert-butoxycarbonyl)-3′-fluoro-[1,1′-biphenyl]-4-yl)carbamoyl)azepane-1-carboxylate

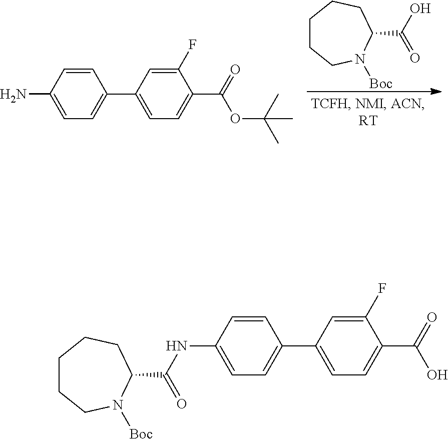 [SMILES: CC(C)(C)OC(=O)c1ccc(-c2ccc(N)cc2)cc1F ; CC(C)(C)OC(=O)N1CCCCC[C@@H]1C(=O)O ; CC(C)(C)OC(=O)N1CCCCC[C@@H]1C(=O)Nc1ccc(-c2ccc(C(=O)O)c(F)c2)cc1]

A solution of (R)-1-(tert-butoxycarbonyl)azepane-2-carboxylic acid (169 mg, 0.7 mmol), tert-butyl 4′-amino-3-fluoro-[1,1′-biphenyl]-4-carboxylate (200 mg, 0.7 mmol), N-(chloro(dimethylamino)methylene)-N-methylmethanaminium hexafluorophosphate(V) (293 mg, 1.0 mmol) and 1-methyl-1H-imidazole (171 mg, 2.1 mmol) in ACN (6 mL) was stirred for 2 hours at room temperature. Upon completion, the resulting mixture was concentrated under reduced pressure. The crude residue was purified by reversed-phase flash chromatography (conditions: column, C18 silica gel; mobile phase A: Water (10 mmol/L NH₄HCO₃), Mobile Phase B: MeCN; Gradient (B %): 5% to 95% in 25 min; detector, UV 254 nm) to give tert-butyl (R)-2-((4′-(tert-butoxycarbonyl)-3′-fluoro-[1,1′-biphenyl]-4-yl)carbamoyl)azepane-1-carboxylate (150 mg, 42% yield) as a yellow solid. LCMS (ESI) [M+H]⁺: 513

### Step 2: (R)-4′-(azepane-2-carboxamido)-3-fluoro-[1,1′-biphenyl]-4-carboxylic acid

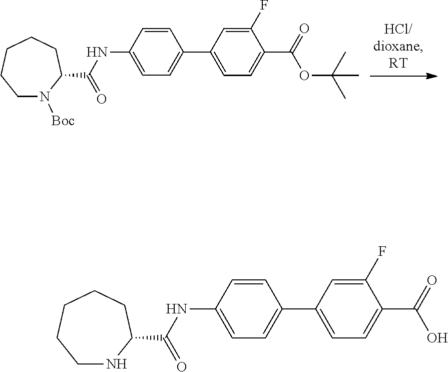 [SMILES: CC(C)(C)OC(=O)c1ccc(-c2ccc(NC(=O)[C@H]3CCCCCN3C(=O)OC(C)(C)C)cc2)cc1F ; O=C(O)c1ccc(-c2ccc(NC(=O)[C@H]3CCCCCN3)cc2)cc1F]

To a mixture of tert-butyl (R)-2-((4′-(tert-butoxycarbonyl)-3′-fluoro-[1,1′-biphenyl]-4-yl)carbamoyl)azepane-1-carboxylate (150 mg, 0.3 mmol) in dioxane (1 mL) was added HCl (20 mmol, 5 mL, 4 M in dioxane) dropwise at 0° C. The resulting mixture was warmed to room temperature and stirred for 2 hours. Upon completion, the resulting mixture was concentrated under reduced pressure to give (R)-4′-(azepane-2-carboxamido)-3-fluoro-[1,1′-biphenyl]-4-carboxylic acid (120 mg, crude) as a light yellow solid, which was used in the next step without further purification. LCMS (ESI) [M+H]⁺: 357.

### Step 3: (R)-3-fluoro-4′-(1-((4-isopropylphenyl)carbamoyl)azepane-2-carboxamido)-[1,1′-biphenyl]-4-carboxylic acid

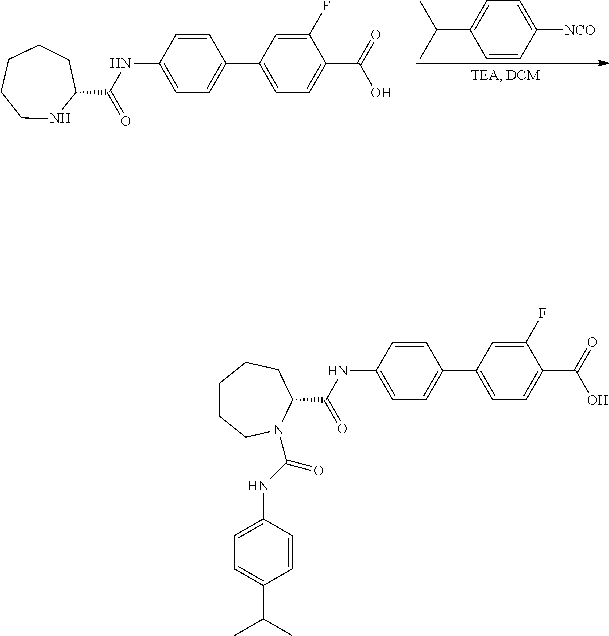

To a mixture of (R)-4′-(azepane-2-carboxamido)-3-fluoro-[1,1′-biphenyl]-4-carboxylic acid (110 mg, 0.3 mmol) in DCM (6 mL) were added triethylamine (31 mg, 0.3 mmol) and 1-isocyanato-4-isopropylbenzene (50 mg, 0.3 mmol) at 0° C. The resulting mixture was warmed to room temperature and stirred for 2 hours. Upon completion, the reaction was quenched with ice water at 0° C. The resulting mixture was extracted with EtOAc (3×8 mL). The combined organic layers were dried over anhydrous Na₂SO₄, filtered and concentrated under reduced pressure. The crude residue was purified by Prep-HPLC (conditions: Column: Xbridge Phenyl OBD Column, 30*150 mm 5 μm; Mobile Phase A: Water (0.1% FA), Mobile Phase B: ACN; Flow rate: 60 mL/min; Gradient (B %): 40% B to 60% B in 15 min; Wave Length: 254 nm/220 nm; RT1 (min): 10.6) to afford (R)-3-fluoro-4′-(1-((4-isopropylphenyl)carbamoyl)azepane-2-carboxamido)-[1,1′-biphenyl]-4-carboxylic acid (25.6 mg, Compound 12; Method C, Peak 2, 2.70 min—analyzed against Compound 11) as a white solid.

| Cmpd | |
|---|---|
| # | Characterization Data |
| 12 | LCMS (ESI) [M + H]⁺: 518.3 |
| | ¹H NMR (400 MHz, DMSO-d₆) δ 13.27 (bs, 1H), 10.12 |
| | (s, 1H), 8.27 (s, 1H), 7.92 (t, J = 8.0 Hz, 1H), 7.80-7.70 (m, |
| | 4H), 7.62-7.59 (m, 2H), 7.40 (d, J = 8.4 Hz, 2H), 7.10 (d, J = |
| | 8.4 Hz, 2H), 4.78-4.74 (m, 1H), 3.98 (d, J = 15.2 Hz, 1H), 3.31- |
| | 3.29 (m, 1H), 2.84-2.81 (m, 1H), 2.34-2.29 (m, 1H), 1.90-1.80 |
| | (m, 1H), 1.77-1.70 (m, 3H), 1.48-1.45 (m, 1H), 1.33-1.28 (m, |
| | 2H), 1.18 (d, J = 6.8 Hz, 6H). |

### Example 13: 3-fluoro-4′-((1S,2S)-2-((4-isopropylphenyl)carbamoyl)cyclopentane-1-carboxamido)-[1,1′-biphenyl]-4-carboxylic acid and 3-fluoro-4′-((1R,2R)-2-((4-isopropylphenyl)carbamoyl)cyclopentane-1-carboxamido)-[1,1′-biphenyl]-4-carboxylic acid

### Step 1: (trans)-2-((4′-(tert-butoxycarbonyl)-3′-fluoro-[1,1′-biphenyl]-4-yl)carbamoyl)cyclopentane-1-carboxylic acid

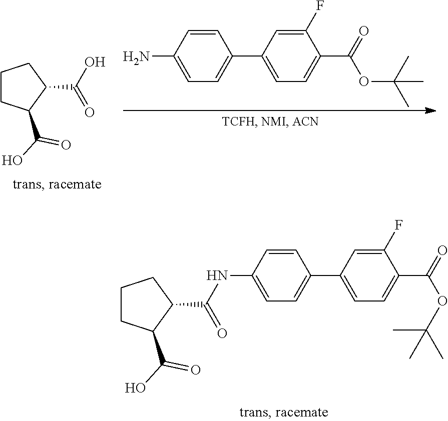 [SMILES: O=C(O)[C@H]1CCC[C@@H]1C(=O)O ; CC(C)(C)OC(=O)c1ccc(-c2ccc(N)cc2)cc1F ; CC(C)(C)OC(=O)c1ccc(-c2ccc(NC(=O)[C@H]3CCC[C@@H]3C(=O)O)cc2)cc1F]

To a mixture of (trans)-cyclopentane-1,2-dicarboxylic acid (300 mg, 1.9 mmol) and tert-butyl 4′-amino-3-fluoro-[1,1′-biphenyl]-4-carboxylate (545 mg, 1.9 mmol) in ACN (6 mL) were added N-(chloro(dimethylamino)methylene)-N-methylmethanaminium hexafluorophosphate(V) (639 mg, 2.3 mmol) and 1-methyl-1H-imidazole (467 mg, 5.7 mmol). The resulting mixture was stirred at room temperature for 1 hour. Upon completion, the resulting mixture was concentrated under reduced pressure. The crude residue was purified by silica gel column chromatography, eluted with PE/EA (1:1) to afford (trans)-2-((4′-(tert-butoxycarbonyl)-3′-fluoro-[1,1′-biphenyl]-4-yl)carbamoyl)cyclopentane-1-carboxylic acid (350 mg, 43% yield) as a white solid. LCMS (ESI) [M+H]⁺: 428

### Step 2: tert-butyl 3-fluoro-4′-((trans)-2-((4-isopropylphenyl)carbamoyl)cyclopentane-1-carboxamido)-[1,1′-biphenyl]-4-carboxylate

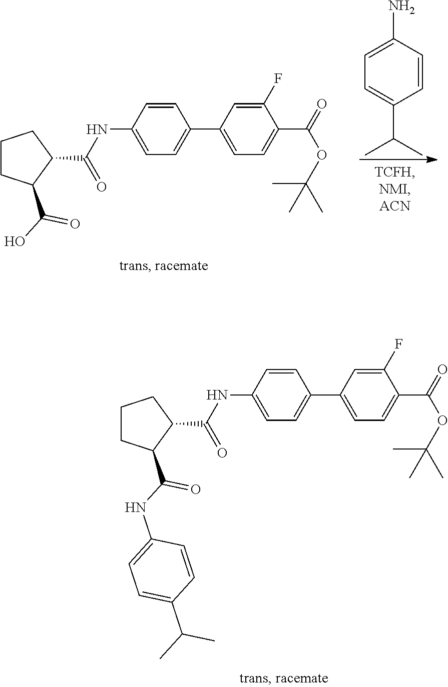

To a mixture of (trans)-2-((4′-(tert-butoxycarbonyl)-3′-fluoro-[1,1′-biphenyl]-4-yl)carbamoyl)cyclopentane-1-carboxylic acid (330 mg, 0.8 mmol) and 4-isopropylaniline (52 mg, 0.4 mmol) in ACN (8 mL) were added N-(chloro(dimethylamino)methylene)-N-methylmethanaminium hexafluorophosphate(V) (217 mg, 0.8 mmol) and 1-methyl-1H-imidazole (190 mg, 2.3 mmol). The resulting mixture was stirred at room temperature for 1 hour. Upon completion, the mixture was concentrated under reduced pressure. The crude residue was purified by silica gel column chromatography, eluted with PE/EA (3:1) to afford tert-butyl 3-fluoro-4′-((trans)-2-((4-isopropylphenyl)carbamoyl)cyclopentane-1-carboxamido)-[1,1′-biphenyl]-4-carboxylate (200 mg, 48% yield) as a white solid. LCMS (ESI) [M+H]⁺: 545

### Step 3: 3-fluoro-4′-((trans)-2-((4-isopropylphenyl)carbamoyl)cyclopentane-1-carboxamido)-[1,1′-biphenyl]-4-carboxylic acid

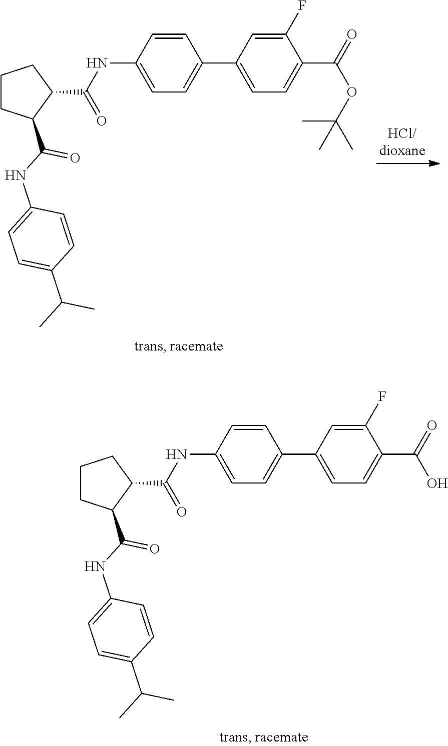 [SMILES: CC(C)c1ccc(NC(=O)[C@H]2CCC[C@@H]2C(=O)Nc2ccc(-c3ccc(C(=O)OC(C)(C)C)c(F)c3)cc2)cc1 ; CC(C)c1ccc(NC(=O)[C@H]2CCC[C@@H]2C(=O)Nc2ccc(-c3ccc(C(=O)O)c(F)c3)cc2)cc1]

To a mixture of tert-butyl 3-fluoro-4′-((trans)-2-((4-isopropylphenyl)carbamoyl)cyclopentane-1-carboxamido)-[1,1′-biphenyl]-4-carboxylate (150 mg, 0.3 mmol) in dioxane (1 mL) was added HCl (20 mmol, 5 mL, 4 M in dioxane) dropwise at 0° C. The resulting mixture was warmed to room temperature and stirred overnight. Upon completion, the resulting mixture was concentrated under reduced pressure. The crude residue was purified by reversed-phase flash chromatography (conditions: column, C18 silica gel; mobile phase A: Water (10 mmol/L NH₄HCO₃), Mobile Phase B: MeCN; Gradient (B %): 5% to 95% in 25 min; detector, UV 254 nm) to afford 3-fluoro-4′-((trans)-2-((4-isopropylphenyl)carbamoyl)cyclopentane-1-carboxamido)-[1,1′-biphenyl]-4-carboxylic acid (36 mg, 27% yield) as a white solid. LCMS (ESI) [M+H]⁺: 489

### Step 3: 3-fluoro-4′-((1S,2S)-2-((4-isopropylphenyl)carbamoyl)cyclopentane-1-carboxamido)-[1,1′-biphenyl]-4-carboxylic acid and 3-fluoro-4′-((1R,2R)-2-((4-isopropylphenyl)carbamoyl)cyclopentane-1-carboxamido)-[1,1′-biphenyl]-4-carboxylic acid

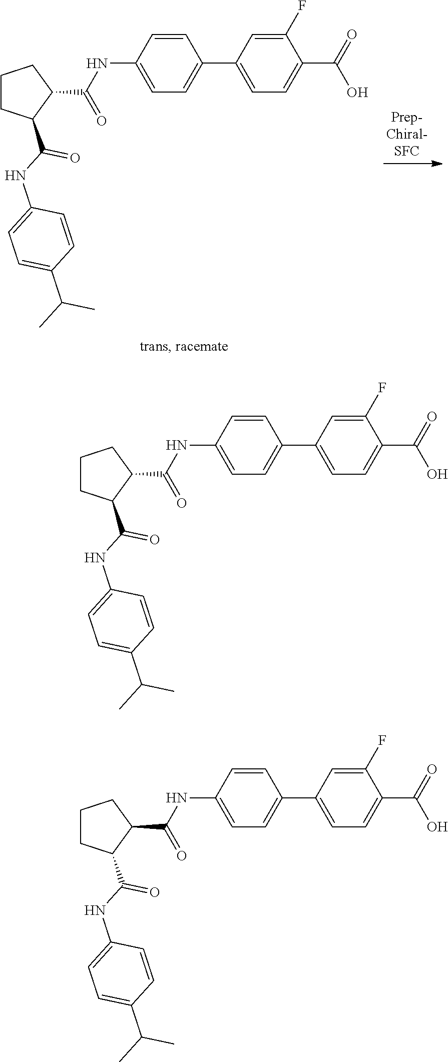 [SMILES: CC(C)c1ccc(NC(=O)[C@H]2CCC[C@@H]2C(=O)Nc2ccc(-c3ccc(C(=O)O)c(F)c3)cc2)cc1 ; CC(C)c1ccc(NC(=O)[C@H]2CCC[C@@H]2C(=O)Nc2ccc(-c3ccc(C(=O)O)c(F)c3)cc2)cc1 ; CC(C)c1ccc(NC(=O)[C@@H]2CCC[C@H]2C(=O)Nc2ccc(-c3ccc(C(=O)O)c(F)c3)cc2)cc1]

3-fluoro-4′-((trans)-2-((4-isopropylphenyl)carbamoyl)cyclopentane-1-carboxamido)-[1,1′-biphenyl]-4-carboxylic acid (34 mg) was separated by Prep-Chiral-SFC (conditions: Column: CHIRAL ART Cellulose-SB, 3*25 cm, 5 μm; Mobile Phase A: CO₂, Mobile Phase B: MeOH (10 mM NH₃); Flow rate: 100 mL/min; Gradient (B %): isocratic 40% B; Column Temperature (° C.): 35; Back Pressure (bar): 100; Wave Length: 254 nm; RT1 (min): 3.92; RT2 (min): 5.5) to afford first peak (Compound 13a) 3-fluoro-4′-((1S,2S)-2-((4-isopropylphenyl)carbamoyl)cyclopentane-1-carboxamido)-[1,1′-biphenyl]-4-carboxylic acid (14.3 mg; Method E, Peak 1, 1.30 min) as a yellow solid and second peak (Compound 13b) 3-fluoro-4′-((1R,2R)-2-((4-isopropylphenyl)carbamoyl)cyclopentane-1-carboxamido)-[1,1′-biphenyl]-4-carboxylic acid (12.1 mg; Method E, Peak 2, 1.49 min) as a yellow solid.

| Cmpd # | Characterization Data |
|---|---|
| 13a | LCMS (ESI) [M + H]⁺: 489.3 |
| | ¹H NMR (400 MHz, DMSO-d₆) δ 10.19 (s, 1H), 9.94 (s, 1H), 7.85-7.83 (m, 1H), 7.75-7.71 (m, |
| | 4H), 7.52 (d, J = 8.0 Hz, 4H), 7.14 (d, J = 8.0 Hz, 2H), 3.22 (s, 2H), 2.87-2.82 (m, 1H), 2.12- |
| | 2.07 (m, 2H), 1.78-1.70 (m, 4H), 1.15 (d, J = 6.8 Hz, 6H). |
| 13b | LCMS (ESI) [M + H]⁺: 489.3 |
| | ¹H NMR (400 MHz, DMSO-d₆) δ10.19 (s, 1H), 9.95 (s, 1H), 8.03-7.72 (m, 5H), 7.69-7.52 (m, |
| | 4H), 7.14 (d, J = 7.2 Hz, 2H), 3.23 (bs, 2H), 2.84 (bs, 1H), 2.09 (bs, 2H), 1.74 (bs, 4H), 1.16 (d, J = |
| | 5.6 Hz, 6H). |

### Example 14: (S)-3-fluoro-4′-(2-(3-(4-isopropylphenyl)-1-methylureido)-3-methylbutanamido)-[1,1′-biphenyl]-4-carboxylic acid

### Step 1: tert-butyl (S)-4′-(2-((tert-butoxycarbonyl)(methyl)amino)-3-methylbutanamido)-3-fluoro-[1,1′-biphenyl]-4-carboxylate

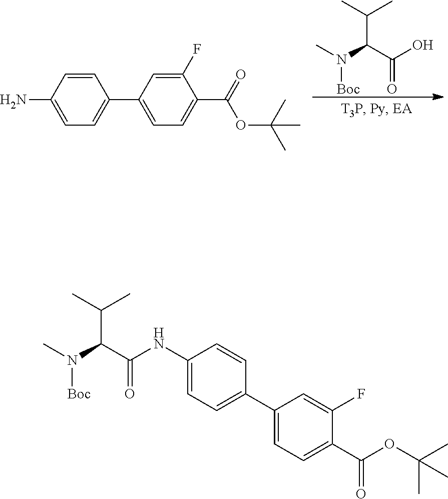

To a mixture of N-(tert-butoxycarbonyl)-N-methyl-L-valine (161 mg, 0.7 mmol) and tert-butyl 4′-amino-3-fluoro-[1,1′-biphenyl]-4-carboxylate (200 mg, 0.7 mmol) in EtOAc (2 mL) was added pyridine (6 mL) and propanephosphonic acid cyclic anhydride (50% in ethyl acetate) (664 mg, 2.0 mmol). The resulting mixture was stirred at room temperature for 2 hours. Upon completion, the mixture was concentrated under reduced pressure. The crude residue was purified by reverse-phase flash chromatography (conditions: column, C18 silica gel; mobile phase A: water (0.1% formic acid), Mobile Phase B: MeCN; Gradient (B %): 0% to 100% in 20 min; detector, UV 254 nm) to give tert-butyl (S)-4′-(2-((tert-butoxycarbonyl)(methyl)amino)-3-methylbutanamido)-3-fluoro-[1,1′-biphenyl]-4-carboxylate (230 mg, 66% yield) as a white solid. LCMS (ESI) [M+H]⁺: 501

### Step 2: (S)-3-fluoro-4′-(3-methyl-2-(methylamino)butanamido)-[1,1′-biphenyl]-4-carboxylic acid

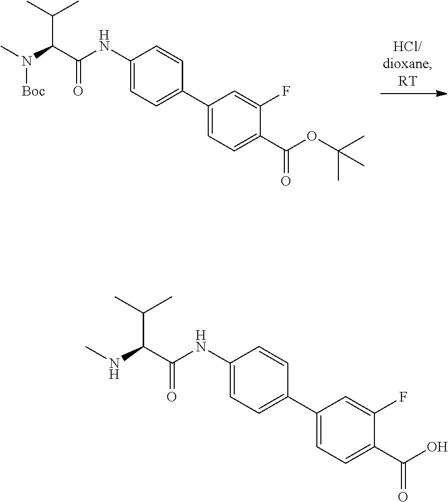

To a mixture of tert-butyl (S)-4′-(2-((tert-butoxycarbonyl)(methyl)amino)-3-methylbutanamido)-3-fluoro-[1,1′-biphenyl]-4-carboxylate (150 mg, 0.3 mmol) in dioxane (0.6 mL) was added HCl (12 mmol, 3 mL, 4 M in dioxane) dropwise at 0° C. The resulting mixture was warmed to room temperature and stirred for 6 hours. Upon completion, the resulting mixture was concentrated under reduced pressure to give (S)-3-fluoro-4′-(3-methyl-2-(methylamino)butanamido)-[1,1′-biphenyl]-4-carboxylic acid (120 mg, crude) as a yellow solid, which was used in the next step without further purification. LCMS (ESI) [M+H]⁺: 345

### Step 3: (S)-3-fluoro-4′-(2-(3-(4-isopropylphenyl)-1-methylureido)-3-methylbutanamido)-[1,1′-biphenyl]-4-carboxylic acid

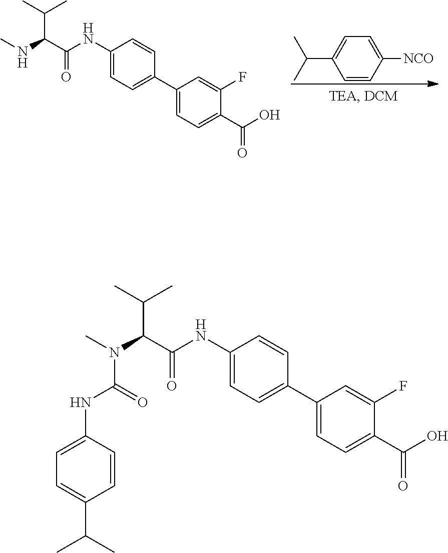

To a mixture of (S)-3-fluoro-4′-(3-methyl-2-(methylamino)butanamido)-[1,1′-biphenyl]-4-carboxylic acid (100 mg, 0.3 mmol) in DCM (4 mL) were added triethylamine (29 mg, 0.3 mmol) and 1-isocyanato-4-isopropylbenzene (46 mg, 0.3 mmol) at 0° C. The resulting mixture was warmed to room temperature and stirred for 2 hours. Upon completion, the reaction was quenched with ice water at 0° C. and the resulting mixture was extracted with EtOAc (3×8 mL). The combined organic layers were dried over anhydrous Na₂SO₄, filtered and concentrated under reduced pressure. The crude residue was purified by Prep-HPLC (conditions: Column: XSelect CSH Fluoro Phenyl 30*150 mm, 5 μm; Mobile Phase A: Water (0.1% FA), Mobile Phase B: MeCN; Flow rate: 60 mL/min; Gradient (B %): 42% B to 60% B in 15 min; Wave Length: 254 nm/220 nm; RT1 (min): 10.2) to afford (S)-3-fluoro-4′-(2-(3-(4-isopropylphenyl)-1-methylureido)-3-methylbutanamido)-[1,1′-biphenyl]-4-carboxylic acid (31.0 mg, Compound 14; Method F, Peak 2, 2.77 min—analyzed against Compound 15) as a white solid.

| Cmpd | |
|---|---|
| # | Characterization Data |
| 14 | LCMS (ESI) [M + H]⁺: 506.2 |
| | ¹H NMR (400 MHz, DMSO-d₆) δ 13.19 (bs, 1H), 10.32 |
| | (s, 1H), 8.31 (s, 1H), 7.94 (t, J = 8.0 Hz, 1H), 7.80-7.77 (m, |
| | 4H), 7.61 (d, J = 7.2 Hz, 2H), 7.41 (d, J = 6.4 Hz, 2H), 7.11 (d, |
| | J = 8.4 Hz, 2H), 4.60 (d, J = 10.8 Hz, 1H), 3.07 (s, 3H), 2.85- |
| | 2.82 (m, 1H), 2.26-2.21 (m, 1H), 1.16 (d, J = 6.8 Hz, 6H), 0.96- |
| | 0.85 (m, 6H). |

### Example 15: (R)-3-fluoro-4′-(2-(3-(4-isopropylphenyl)-1-methylureido)-3-methylbutanamido)-[1,1′-biphenyl]-4-carboxylic acid

### Step 1: tert-butyl (R)-4′-(2-((tert-butoxycarbonyl)(methyl)amino)-3-methylbutanamido)-3-fluoro-[1,1′-biphenyl]-4-carboxylate

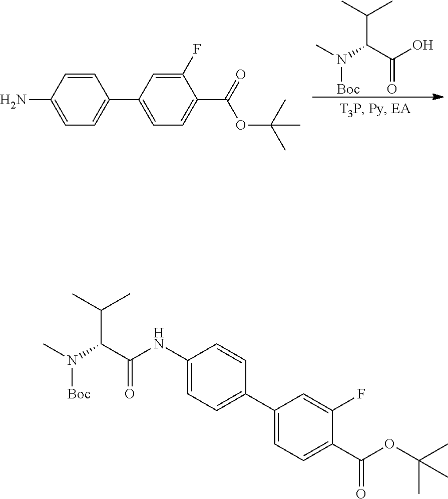

To a mixture of N-(tert-butoxycarbonyl)-N-methyl-D-valine (200 mg, 0.9 mmol) and tert-butyl 4′-amino-3-fluoro-[1,1′-biphenyl]-4-carboxylate (298 mg, 1.0 mmol) in EtOAc (3 mL) was added pyridine (9 mL) and propanephosphonic acid cyclic anhydride (50% in ethyl acetate) (3.30 g, 5.2 mmol) dropwise at 0° C. The resulting mixture was warmed to room temperature and stirred for 2 hours. Upon completion, the resulting mixture was concentrated under reduced pressure. The crude residue was purified by reversed-phase flash chromatography (conditions: column, C18 silica gel; mobile phase A: water (10 mmol/L NH₄HCO₃), Mobile Phase B: MeCN: Gradient (B %): 0% to 100% in 25 min; detector, UV 254 nm) to give tert-butyl (R)-4′-(2-((tert-butoxycarbonyl)(methyl)amino)-3-methylbutanamido)-3-fluoro-[1,1′-biphenyl]-4-carboxylate (150 mg, 35% yield) as a yellow solid. LCMS (ESI) [M+H]⁺: 501

### Step 2: (R)-3-fluoro-4′-(3-methyl-2-(methylamino)butanamido)-[1,1′-biphenyl]-4-carboxylic acid

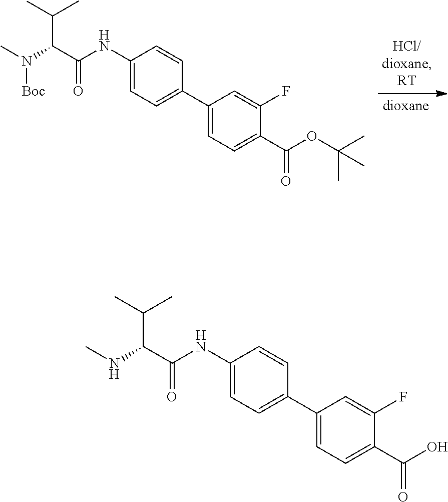

To a mixture of tert-butyl (R)-4′-(2-((tert-butoxycarbonyl)(methyl)amino)-3-methylbutanamido)-3-fluoro-[1,1′-biphenyl]-4-carboxylate (150 mg, 0.3 mmol) in dioxane (0.6 mL) was added HCl (12 mmol, 3 mL, 4 M in dioxane) dropwise at 0° C. The resulting mixture was warmed to room temperature and stirred for 6 hours. Upon completion, the resulting mixture was concentrated under reduced pressure to give (R)-3-fluoro-4′-(3-methyl-2-(methylamino)butanamido)-[1,1′-biphenyl]-4-carboxylic acid (120 mg, crude) as a yellow solid, which was used in the next step without further purification. LCMS (ESI) [M+H]⁺: 345

### Step 3: (R)-3-fluoro-4′-(2-(3-(4-isopropylphenyl)-1-methylureido)-3-methylbutanamido)-[1,1′-biphenyl]-4-carboxylic acid

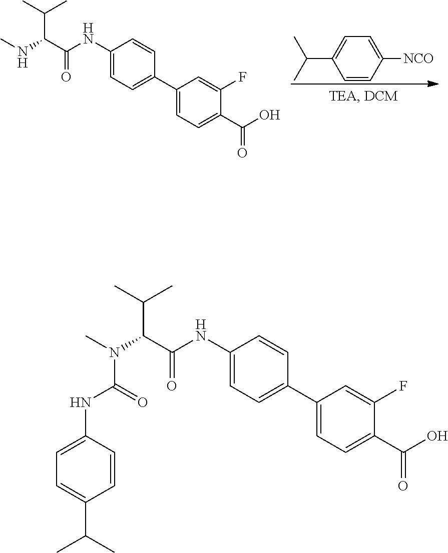

To a mixture of (R)-3-fluoro-4′-(3-methyl-2-(methylamino)butanamido)-[1,1′-biphenyl]-4-carboxylic acid (110 mg, 0.3 mmol) in DCM (5 mL) were added triethylamine (32 mg, 0.3 mmol) and 1-isocyanato-4-isopropylbenzene (52 mg, 0.3 mmol) under nitrogen atmosphere. The resulting mixture was stirred at room temperature for 2 hours. Upon completion, the reaction was quenched with ice water at 0° C. The resulting mixture was extracted with EtOAc (3×8 mL). The combined organic layers were dried over anhydrous Na₂SO₄, filtered and concentrated under reduced pressure. The crude residue was purified by Prep-HPLC (conditions: Column: Xbridge Phenyl OBD Column, 30*150 mm 5 μm; Mobile Phase A: Water (0.1% FA), Mobile Phase B: ACN; Flow rate: 60 mL/min; Gradient (B %): 40% B to 60% B in 15 min; Wave Length: 254 nm/220 nm; RT1 (min): 10.6) to afford (R)-3-fluoro-4′-(2-(3-(4-isopropylphenyl)-1-methylureido)-3-methylbutanamido)-[1,1′-biphenyl]-4-carboxylic acid (31.2 mg, Compound 15; Method F, Peak 1, 2.66 min—analyzed against Compound 14) as a white solid.

| Cmpd | |
|---|---|
| # | Characterization Data |
| 15 | LCMS (ESI) [M + H]⁺: 506.3 |
| | ¹H NMR (400 MHz, DMSO-d₆) δ 13.22 (bs, 1H), 10.34 |
| | (s, 1H), 8.32 (s, 1H), 7.94 (t, J = 8.0 Hz, 1H), 7.91-7.89 (m, |
| | 4H), 7.79-7.76 (m, 2H), 7.41 (d, J = 6.4 Hz, 2H), 7.11 (d, J = |
| | 8.4 Hz, 2H), 4.60 (d, J = 10.8 Hz, 1H), 3.07 (s, 3H), 2.85-2.82 |
| | (m, 1H), 2.26-2.22 (m, 1H), 1.18 (d, J = 6.8 Hz, 6H), |
| | 0.96-0.88 (m, 6H). |

### Example 16: 3-fluoro-4′-(1-((4-isopropylphenyl)carbamoyl)-3,3-dimethylazetidine-2-carboxamido)-[1,1′-biphenyl]-4-carboxylic acid

### Step 1: tert-butyl 2-((4′-(tert-butoxycarbonyl)-3′-fluoro-[1,1′-biphenyl]-4-yl)carbamoyl)-3,3-dimethylazetidine-1-carboxylate

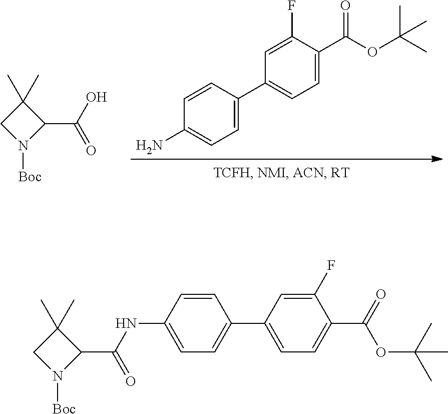 [SMILES: CC(C)(C)OC(=O)N1CC(C)(C)C1C(=O)O ; CC(C)(C)OC(=O)c1ccc(-c2ccc(N)cc2)cc1F ; CC(C)(C)OC(=O)c1ccc(-c2ccc(NC(=O)C3N(C(=O)OC(C)(C)C)CC3(C)C)cc2)cc1F]

A solution of 1-(tert-butoxycarbonyl)-3,3-dimethylazetidine-2-carboxylic acid (90 mg, 0.3 mmol), tert-butyl 4′-amino-3-fluoro-[1,1′-biphenyl]-4-carboxylate (118 mg, 0.4 mmol), 1-methyl-1H-imidazole (161 mg, 1.9 mmol) and N-(chloro(dimethylamino)methylene)-N-methylmethanaminium hexafluorophosphate(V) (115 mg, 0.4 mmol) in ACN (3 mL) was stirred at room temperature for 2 hours. Upon completion, the resulting mixture was concentrated under reduced pressure. The crude residue was purified by reversed-phase flash chromatography (conditions: column, C18 silica gel; mobile phase A: water (10 mmol/L NH₄HCO₃), Mobile Phase B: MeCN; Gradient (B %): 5% to 95% in 25 min; detector, UV 300 nm) to give tert-butyl 2-((4′-(tert-butoxycarbonyl)-3′-fluoro-[1,1′-biphenyl]-4-yl)carbamoyl)-3,3-dimethylazetidine-1-carboxylate (100 mg, 51% yield) as a white solid. LCMS (ESI) [M+H]⁺: 499

### Step 2: 4′-(3,3-dimethylazetidine-2-carboxamido)-3-fluoro-[1,1′-biphenyl]-4-carboxylic acid

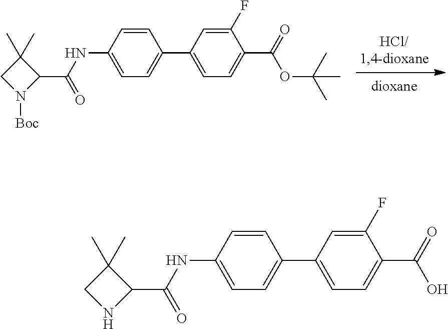 [SMILES: CC(C)(C)OC(=O)c1ccc(-c2ccc(NC(=O)C3N(C(=O)OC(C)(C)C)CC3(C)C)cc2)cc1F ; CC1(C)CNC1C(=O)Nc1ccc(-c2ccc(C(=O)O)c(F)c2)cc1]

To a solution of tert-butyl 2-((4′-(tert-butoxycarbonyl)-3′-fluoro-[1,1′-biphenyl]-4-yl)carbamoyl)-3,3-dimethylazetidine-1-carboxylate (90 mg, 0.2 mmol) in dioxane (0.6 mL) was added HCl (12 mmol, 3 mL, 4 M in dioxane) dropwise at 0° C. The resulting mixture was warmed to room temperature and stirred overnight. Upon completion, the resulting mixture was concentrated under reduced pressure to give 4′-(3,3-dimethylazetidine-2-carboxamido)-3-fluoro-[1,1′-biphenyl]-4-carboxylic acid (100 mg, crude) as a yellow solid, which was used in the next step without further purification. LCMS (ESI) [M+H]⁺: 343

### Step 3: 3-fluoro-4′-(1-((4-isopropylphenyl)carbamoyl)-3,3-dimethylazetidine-2-carboxamido)-[1,1′-biphenyl]-4-carboxylic acid

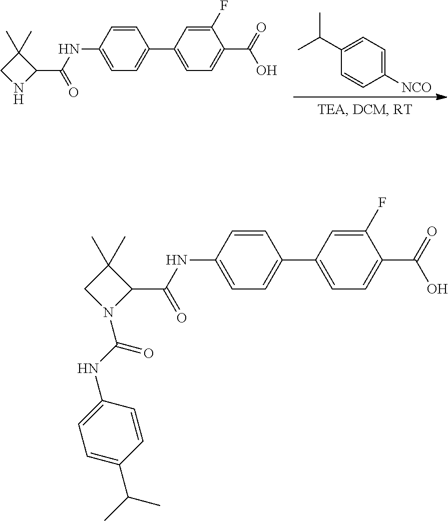

To a mixture of 4′-(3,3-dimethylazetidine-2-carboxamido)-3-fluoro-[1,1′-biphenyl]-4-carboxylic acid (90 mg, 0.2 mmol) in DCM (3 mL) were added triethylamine (36 mg, 0.3 mmol) and 1-isocyanato-4-isopropylbenzene (45 mg, 0.2 mmol) at 0° C. The resulting mixture was warmed to room temperature and stirred for 2 hours. Upon completion, the reaction was quenched with ice water at 0° C. The resulting mixture was extracted with EtOAc (3×6 mL). The combined organic layers were dried over anhydrous Na₂SO₄, filtered and concentrated under reduced pressure. The crude residue was purified by Prep-HPLC (conditions: Column: Xselect CSH Prep C18, 30*150 mm 5 μm; Mobile Phase A: Water (0.1% FA), Mobile Phase B: MeCN; Flow rate: 60 mL/min; Gradient (B %): 42% to 60% B in 12 min; Wave Length: 254 nm/220 nm; RT1 (min): 12.50) to afford 3-fluoro-4′-(1-((4-isopropylphenyl)carbamoyl)-3,3-dimethylazetidine-2-carboxamido)-[1,1′-biphenyl]-4-carboxylic acid (31 mg, 25% yield, Compound 16) as a white solid.

| Cmpd | |
|---|---|
| # | Characterization Data |
| 16 | LCMS (ESI) [M + H]⁺: 504.2 |
| | ¹H NMR (400 MHz, DMSO-d₆) δ 13.22 (bs, 1H), 10.02 |
| | (s, 1H), 8.45 (s, 1H), 7.93 (t, J = 8.2 Hz, 1H), 7.80-7.75 (m, |
| | 4H), 7.63-7.60 (m, 2H), 7.42 (d, J = 8.4 Hz, 2H), 7.11 (d, J = |
| | 8.4 Hz, 2H), 4.40 (s, 1H), 3.76 (d, J = 7.2 Hz, 1H), 3.74 (d, J = |
| | 7.6 Hz, 1H), 2.84-2.80 (m, 1H), 1.38 (s, 3H), 1.18-1.12 (m, |
| | 9H). |

### Example 17: 3-fluoro-4′-((1R,2R)-2-((4-isopropylphenyl)carbamoyl)cyclohexane-1-carboxamido)-[1,1′-biphenyl]-4-carboxylic acid

### Step 1: (1R,2R)-2-((4′-(tert-butoxycarbonyl)-3′-fluoro-[1,1′-biphenyl]-4-yl)carbamoyl)cyclohexane-1-carboxylic acid

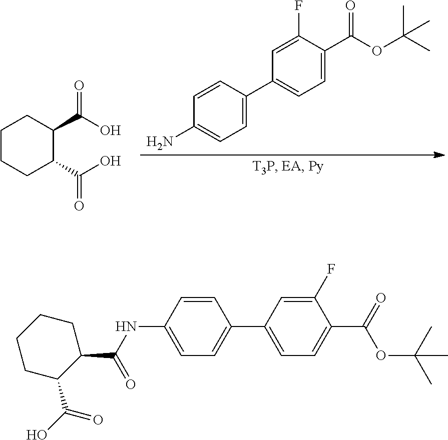 [SMILES: O=C(O)[C@@H]1CCCC[C@H]1C(=O)O ; CC(C)(C)OC(=O)c1ccc(-c2ccc(N)cc2)cc1F ; CC(C)(C)OC(=O)c1ccc(-c2ccc(NC(=O)[C@@H]3CCCC[C@H]3C(=O)O)cc2)cc1F]

To a mixture of (1R,2R)-cyclohexane-1,2-dicarboxylic acid (200 mg, 1.1 mmol) and tert-butyl 4′-amino-3-fluoro-[1,1′-biphenyl]-4-carboxylate (300 mg, 1.0 mmol) in EtOAc (3 mL) was added pyridine (9 mL) and propanephosphonic acid cyclic anhydride (50% in ethyl acetate) (2.20 g, 3.4 mmol) dropwise at 0° C. The resulting mixture was warmed to room temperature and stirred for 2 hours. Upon completion, the resulting mixture was concentrated under reduced pressure. The crude residue was purified by silica gel column chromatography, eluted with PE/EA (1:1) to afford (1R,2R)-2-((4′-(tert-butoxycarbonyl)-3′-fluoro-[1,1′-biphenyl]-4-yl)carbamoyl)cyclohexane-1-carboxylic acid (200 mg, 39% yield) as a yellow solid. LCMS (ESI) [M+H]⁺: 442

### Step 2: tert-butyl 3-fluoro-4′-((1R,2R)-2-((4-isopropylphenyl)carbamoyl)cyclohexane-1-carboxamido)-[1,1′-biphenyl]-4-carboxylate

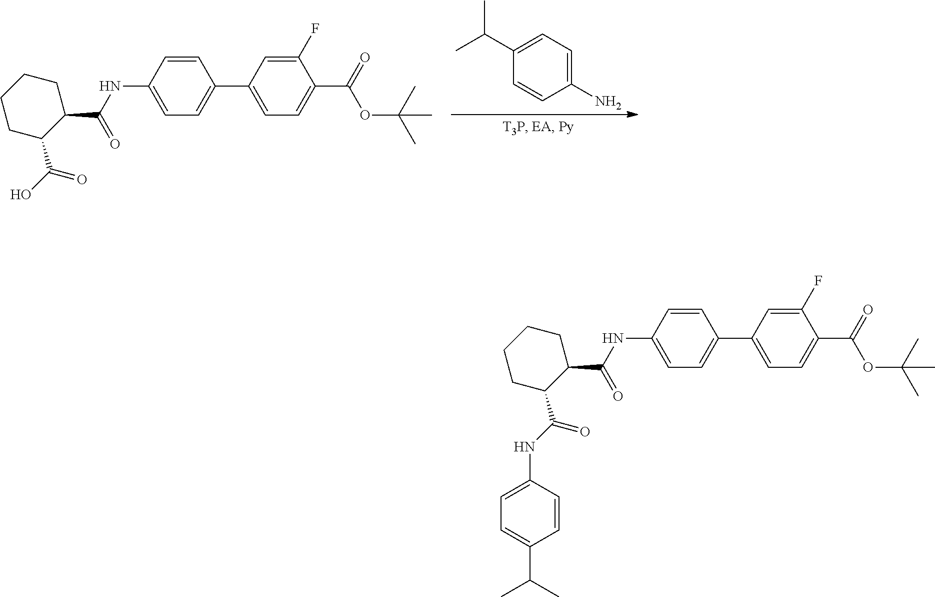

To a mixture of (1R,2R)-2-((4′-(tert-butoxycarbonyl)-3′-fluoro-[1,1′-biphenyl]-4-yl)carbamoyl)cyclohexane-1-carboxylic acid (180 mg, 0.4 mmol) and 4-isopropylaniline (110 mg, 0.8 mmol) in EtOAc (2 mL) was added pyridine (6 mL) and propanephosphonic acid cyclic anhydride (50% in ethyl acetate) (778 mg, 1.2 mmol) dropwise at 0° C. The resulting mixture was warmed to room temperature and stirred for 2 hours. Upon completion, the resulting mixture was concentrated under reduced pressure. The crude residue was purified by reversed-phase flash chromatography (conditions: column, C18 silica gel; mobile phase A: Water (0.1% formic acid), Mobile Phase B: MeCN; Gradient (B %): 5% to 95% in 25 min; detector, UV 300 nm) to give tert-butyl 3-fluoro-4′-((1R,2R)-2-((4-isopropylphenyl)carbamoyl)cyclohexane-1-carboxamido)-[1,1′-biphenyl]-4-carboxylate (150 mg, 65% yield) as a white solid. LCMS (ESI) [M+H]⁺: 559

### Step 3: 3-fluoro-4′-((1R,2R)-2-((4-isopropylphenyl)carbamoyl)cyclohexane-1-carboxamido)-[1,1′-biphenyl]-4-carboxylic acid

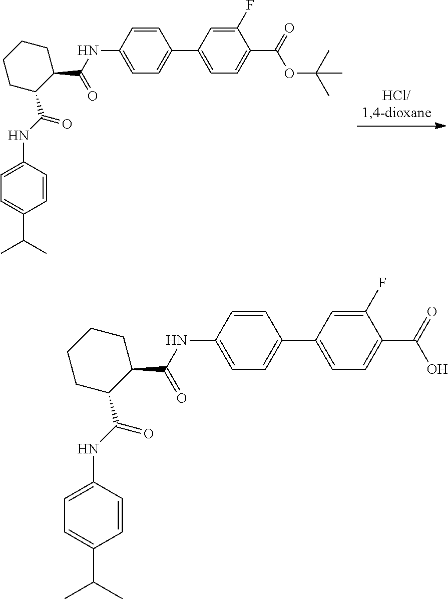 [SMILES: CC(C)c1ccc(NC(=O)[C@@H]2CCCC[C@H]2C(=O)Nc2ccc(-c3ccc(C(=O)OC(C)(C)C)c(F)c3)cc2)cc1 ; CC(C)c1ccc(NC(=O)[C@@H]2CCCC[C@H]2C(=O)Nc2ccc(-c3ccc(C(=O)O)c(F)c3)cc2)cc1]

To a mixture of tert-butyl 3-fluoro-4′-((1R,2R)-2-((4-isopropylphenyl)carbamoyl)cyclohexane-1-carboxamido)-[1,1′-biphenyl]-4-carboxylate (80 mg, 0.1 mmol) in dioxane (0.6 mL) was added HCl (12 mmol, 3 mL, 4 M in dioxane) dropwise at 0° C. The resulting mixture was warmed to room temperature and stirred for 1 hour. Upon completion, the resulting mixture was concentrated under reduced pressure. The crude residue was purified by Prep-HPLC (conditions: Column: Xselect CSH Prep C18, 30*150 mm 5 μm; Mobile Phase A: Water (0.1% FA), Mobile Phase B: MeCN; Flow rate: 60 mL/min; Gradient (B %): 46% to 64% B in 12 min; Wave Length: 254 nm/220 nm; RT1 (min): 14.43) to afford 3-fluoro-4′-((1R,2R)-2-((4-isopropylphenyl)carbamoyl)cyclohexane-1-carboxamido)-[1,1′-biphenyl]-4-carboxylic acid (26.7 mg, Compound 17; Method E, Peak 2, 1.49 min—analyzed against Compound 18) as a white solid.

| Cmpd # | Characterization Data |
|---|---|
| 17 | LCMS (ESI) [M + H]⁺: 503.2 |
| | ¹H NMR (400 MHz, DMSO-d₆) δ 13.16 (bs, 1H), 10.14 (s, 1H), 9.88 (s, 1H), 7.91 (t, J = 8.0 Hz, |
| | 1H), 7.71 (s, 4H), 7.58 (d, J = 9.6 Hz, 2H), 7.47 (d, J = 8.4 Hz, 2H), 7.11 (d, J = 8.4 Hz, 2H), 2.82- |
| | 2.73 (m, 3H), 2.09 (bs, 2H), 1.82 (bs, 2H), 1.39-1.32 (m, 4H), 1.15 (d, J = 6.8 Hz, 6H). |

### Example 18: 3-fluoro-4′-((1S,2S)-2-((4-isopropylphenyl)carbamoyl)cyclohexane-1-carboxamido)-[1,1′-biphenyl]-4-carboxylic acid

### Step 1: (1S,2S)-2-((4′-(tert-butoxycarbonyl)-3′-fluoro-[1,1′-biphenyl]-4-yl)carbamoyl)cyclohexane-1-carboxylic acid

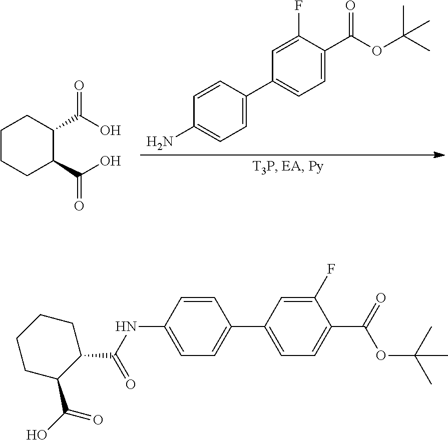 [SMILES: O=C(O)[C@H]1CCCC[C@@H]1C(=O)O ; CC(C)(C)OC(=O)c1ccc(-c2ccc(N)cc2)cc1F ; CC(C)(C)OC(=O)c1ccc(-c2ccc(NC(=O)[C@H]3CCCC[C@@H]3C(=O)O)cc2)cc1F]

To a mixture of (1S,2S)-cyclohexane-1,2-dicarboxylic acid (200 mg, 1.2 mmol) and tert-butyl 4′-amino-3-fluoro-[1,1′-biphenyl]-4-carboxylate (413 mg, 1.4 mmol) in EtOAc (3 mL) was added pyridine (9 mL) and propanephosphonic acid cyclic anhydride (50% in ethyl acetate) (1.91 g, 6.0 mmol) dropwise at 0° C. The resulting mixture was warmed to room temperature and stirred for 2 hours. Upon completion, the resulting mixture was concentrated under reduced pressure. The crude residue was purified by silica gel column chromatography, eluted with PE/EA (2:3) to afford (1S,2S)-2-((4′-(tert-butoxycarbonyl)-3′-fluoro-[1,1′-biphenyl]-4-yl)carbamoyl)cyclohexane-1-carboxylic acid (220 mg, 43% yield) as a yellow solid. LCMS (ESI) [M+H]⁺: 442

### Step 2: tert-butyl 3-fluoro-4′-((1S,2S)-2-((4-isopropylphenyl)carbamoyl)cyclohexane-1-carboxamido)-[1,1′-biphenyl]-4-carboxylate

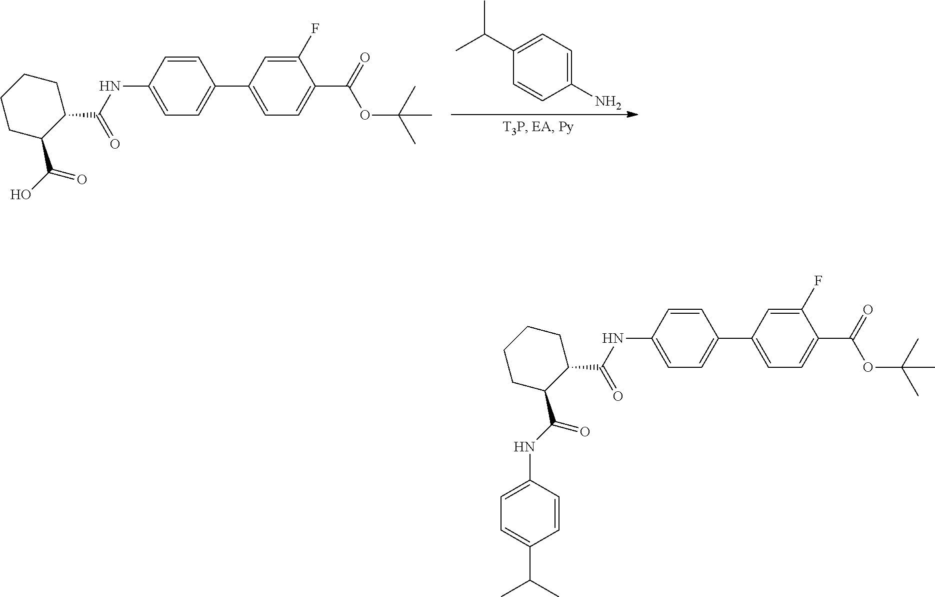

To a mixture of (1S,2S)-2-((4′-(tert-butoxycarbonyl)-3′-fluoro-[1,1′-biphenyl]-4-yl)carbamoyl)cyclohexane-1-carboxylic acid (220 mg, 0.5 mmol) and 4-isopropylaniline (1.27 g, 2.0 mmol) in EtOAc (3 mL) was added pyridine (9 mL) and propanephosphonic acid cyclic anhydride (50% in ethyl acetate) (793 mg, 2.5 mmol) dropwise at 0° C. The resulting mixture was warmed to room temperature and stirred for 2 hours. Upon completion, the resulting mixture was concentrated under reduced pressure. The crude residue was purified by reversed-phase flash chromatography (conditions: column, C18 silica gel; mobile phase A: Water (0.1% formic acid), Mobile Phase B: MeCN; Gradient (B %): 5% to 95% in 25 min; detector, UV 254 nm) to yield tert-butyl 3-fluoro-4′-((1S,2S)-2-((4-isopropylphenyl)carbamoyl)cyclohexane-1-carboxamido)-[1,1′-biphenyl]-4-carboxylate (160 mg, 58% yield) as a white solid. LCMS (ESI) [M+H]⁺: 559

### Step 3: 3-fluoro-4′-((1S,2S)-2-((4-isopropylphenyl)carbamoyl)cyclohexane-1-carboxamido)-[1,1′-biphenyl]-4-carboxylic acid

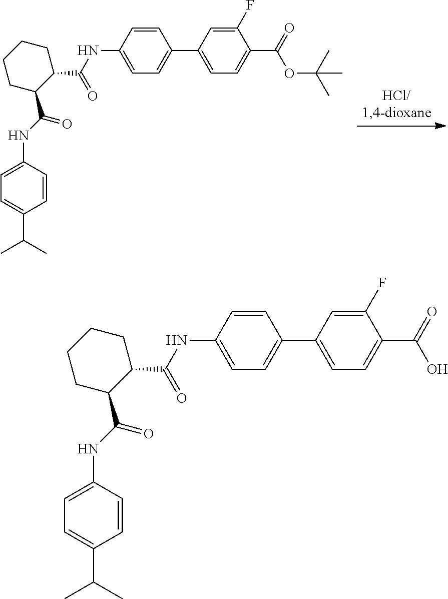 [SMILES: CC(C)c1ccc(NC(=O)[C@H]2CCCC[C@@H]2C(=O)Nc2ccc(-c3ccc(C(=O)OC(C)(C)C)c(F)c3)cc2)cc1 ; CC(C)c1ccc(NC(=O)[C@H]2CCCC[C@@H]2C(=O)Nc2ccc(-c3ccc(C(=O)O)c(F)c3)cc2)cc1]

To a mixture of tert-butyl 3-fluoro-4′-((1S,2S)-2-((4-isopropylphenyl)carbamoyl)cyclohexane-1-carboxamido)-[1,1′-biphenyl]-4-carboxylate (100 mg, 0.2 mmol) in dioxane (0.6 mL) was added HCl (12 mmol, 3 mL, 4 M in dioxane) dropwise at 0° C. The resulting mixture was warmed to room temperature and stirred for 1 hour. Upon completion, the resulting mixture was concentrated under reduced pressure. The crude residue was purified by Prep-HPLC (conditions: Column: Xselect CSH Prep C18, 30*150 mm 5 μm; Mobile Phase A: Water (0.1% FA), Mobile Phase B: ACN; Flow rate: 60 mL/min; Gradient (B %): 45% to 65% B in 10 min; Wave Length: 254 nm/220 nm; RT1 (min): 12.40) to afford 3-fluoro-4′-((1S,2S)-2-((4-isopropylphenyl)carbamoyl)cyclohexane-1-carboxamido)-[1,1′-biphenyl]-4-carboxylic acid (33.5 mg, Compound 18; Method E, Peak 1, 1.25 min—analyzed against Compound 17) as an off-white solid.

| Cmpd # | Characterization Data |
|---|---|
| 18 | LCMS (ESI) [M + H]⁺: 503.3 |
| | ¹H NMR (400 MHz, DMSO-d₆) δ 13.13 (bs, 1H), 10.17 (s, 1H), 9.89 (s, 1H), 7.91 (t, J = 8.0 Hz, |
| | 1H), 7.70 (s, 4H), 7.56 (d, J = 10.4 Hz, 2H), 7.48 (d, J = 8.4 Hz, 2H), 7.11 (d, J = 8.4 Hz, 2H), |
| | 2.82-2.73 (m, 3H), 1.99 (bs, 2H), 1.81 (bs, 2H), 1.33-1.21 (m, 4H), 1.13 (d, J = 7.2 Hz, 6H). |

### Example 19: 3-fluoro-4′-(1-(3-(4-isopropylphenyl)-1-methylureido)cyclopentane-1-carboxamido)-[1,1′-biphenyl]-4-carboxylic acid

### Step 1: tert-butyl 4′-(1-((tert-butoxycarbonyl)amino)cyclopentane-1-carboxamido)-3-fluoro-[1,1′-biphenyl]-4-carboxylate

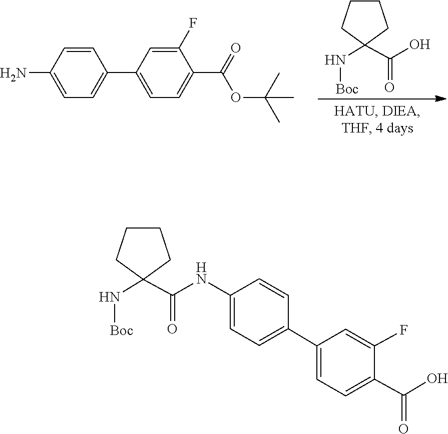 [SMILES: CC(C)(C)OC(=O)c1ccc(-c2ccc(N)cc2)cc1F ; CC(C)(C)OC(=O)NC1(C(=O)O)CCCC1 ; CC(C)(C)OC(=O)NC1(C(=O)Nc2ccc(-c3ccc(C(=O)O)c(F)c3)cc2)CCCC1]

To a mixture of 1-((tert-butoxycarbonyl)amino)cyclopentane-1-carboxylic acid (239 mg, 1.0 mmol) in THF (7 mL) were added DIEA (270 mg, 2.1 mmol), tert-butyl 4′-amino-3-fluoro-[1,1′-biphenyl]-4-carboxylate (300 mg, 1.0 mmol) and HATU (437 mg, 1.1 mmol). The resulting mixture was stirred at room temperature for 4 days. Upon completion, the resulting mixture was concentrated under reduced pressure. The crude residue was purified by silica gel column chromatography, eluted with PE/EA (1:1) to afford tert-butyl 4′-(1-((tert-butoxycarbonyl)amino)cyclopentane-1-carboxamido)-3-fluoro-[1,1′-biphenyl]-4-carboxylate (300 mg, 58% yield) as a yellow solid. LCMS (ESI) [M+H]⁺: 499

### Step 2: 4′-(1-aminocyclopentane-1-carboxamido)-3-fluoro-[1,1′-biphenyl]-4-carboxylic acid

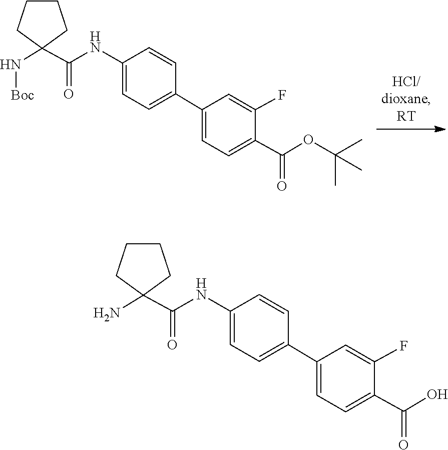 [SMILES: CC(C)(C)OC(=O)NC1(C(=O)Nc2ccc(-c3ccc(C(=O)OC(C)(C)C)c(F)c3)cc2)CCCC1 ; NC1(C(=O)Nc2ccc(-c3ccc(C(=O)O)c(F)c3)cc2)CCCC1]

To a mixture of tert-butyl 4′-(1-((tert-butoxycarbonyl)amino)cyclopentane-1-carboxamido)-3-fluoro-[1,1′-biphenyl]-4-carboxylate (300 mg, 0.6 mmol) in dioxane (1 mL) was added HCl (20 mmol, 5 mL, 4 M in dioxane) dropwise at 0° C. The resulting mixture was warmed to room temperature and stirred for 12 hours. Upon completion, the resulting mixture was concentrated under reduced pressure to yield 4′-(1-aminocyclopentane-1-carboxamido)-3-fluoro-[1,1′-biphenyl]-4-carboxylic acid (220 mg, crude) as a yellow solid, which was used in the next step without further purification. LCMS (ESI) [M+H]⁺: 343

### Step 3: 3-fluoro-4′-(1-(methylamino)cyclopentane-1-carboxamido)-[1,1′-biphenyl]-4-carboxylic acid

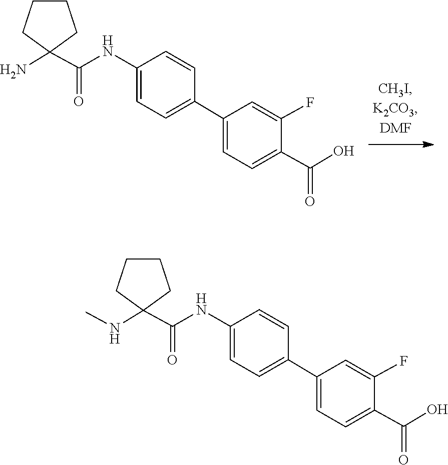 [SMILES: NC1(C(=O)Nc2ccc(-c3ccc(C(=O)O)c(F)c3)cc2)CCCC1 ; CNC1(C(=O)Nc2ccc(-c3ccc(C(=O)O)c(F)c3)cc2)CCCC1]

To a mixture of 4′-(1-aminocyclopentane-1-carboxamido)-3-fluoro-[1,1′-biphenyl]-4-carboxylic acid (200 mg, 0.6 mmol) in DMF (6 mL) were added K₂CO₃ (242 mg, 1.8 mmol) and methyl iodide (83 mg, 0.6 mmol). The resulting mixture was stirred at room temperature for 1 hour. Upon completion, the resulting mixture was diluted with H₂O (8 mL) and extracted with EtOAc (3×10 mL). The combined organic layers were dried over anhydrous Na₂SO₄, filtered and concentrated under reduced pressure. The crude residue was purified by silica gel column chromatography, eluted with PE/EA (2:3) to afford 3-fluoro-4′-(1-(methylamino)cyclopentane-1-carboxamido)-[1,1′-biphenyl]-4-carboxylic acid (150 mg, 72% yield) as a yellow solid. LCMS (ESI) [M+H]⁺: 357

### Step 4: 3-fluoro-4′-(1-(3-(4-isopropylphenyl)-1-methylureido)cyclopentane-1-carboxamido)-[1,1′-biphenyl]-4-carboxylic acid

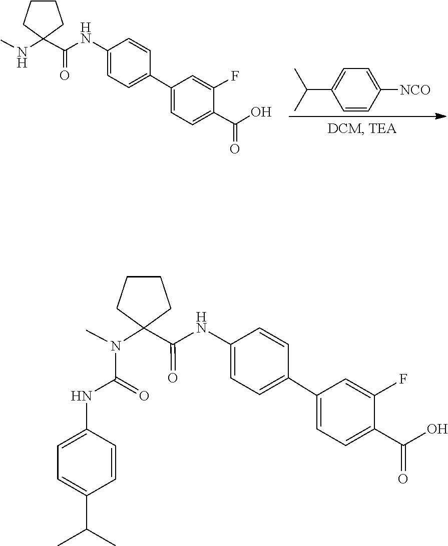

To a mixture of 3-fluoro-4′-(1-(methylamino)cyclopentane-1-carboxamido)-[1,1′-biphenyl]-4-carboxylic acid (100 mg, 0.3 mmol) in DCM (3 mL) were added triethylamine (28 mg, 0.3 mmol) and 1-isocyanato-4-isopropylbenzene (45 mg, 0.3 mmol) dropwise under nitrogen atmosphere. The resulting mixture was stirred at room temperature for 2 hours. Upon completion, the reaction was quenched with ice water at 0° C. The resulting mixture was extracted with EtOAc (3×6 mL). The combined organic layers were dried over anhydrous Na₂SO₄, filtered and concentrated under reduced pressure. The crude residue was purified by Prep-HPLC (conditions: Column: XSelect CSH Fluoro Phenyl 30*150 mm, 5 μm; Mobile Phase A: Water (0.1% FA), Mobile Phase B: ACN; Flow rate: 60 mL/min; Gradient (B %): 41% B to 58% B in 15 min; Wave Length: 254 nm/220 nm; RT1 (min): 10.8) to afford 3-fluoro-4′-(1-(3-(4-isopropylphenyl)-1-methylureido)cyclopentane-1-carboxamido)-[1,1′-biphenyl]-4-carboxylic acid (35.2 mg, 24% yield, Compound 19) as a white solid.

| Cmpd # | Characterization Data |
|---|---|
| 19 | LCMS (ESI) [M + H]⁺: 518.2 |
| | ¹H NMR (400 MHz, DMSO-d₆) δ 7.99-7.95 (m, 1H), 7.71-7.66 (m, 4H), 7.65-7.55 (m, 2H), 7.26 |
| | (d, J = 13.2 Hz, 2H), 7.12 (d, J = 8.4 Hz, 2H), 4.84 (s, 3H), 2.84-2.82 (m, 1H), 2.37-2.34 (m, 2H), |
| | 2.02-1.97 (m, 2H), 1.86-1.82 (m, 4H), 1.18 (d, J = 6.8 Hz, 6H). |

### Example 20: 4-(6-((S)-1-((4-((S)-2,2-dimethylcyclopropyl)phenyl)carbamoyl)pyrrolidin-2-yl)pyridin-3-yl)benzoic acid, 4-(6-((R)-1-((4-((S)-2,2-dimethylcyclopropyl)phenyl)carbamoyl)pyrrolidin-2-yl)pyridin-3-yl)benzoic acid, 4-(6-((S)-1-((4-((R)-2,2-dimethylcyclopropyl)phenyl)carbamoyl)pyrrolidin-2-yl)pyridin-3-yl)benzoic acid and 4-(6-((R)-1-((4-((R)-2,2-dimethylcyclopropyl)phenyl)carbamoyl)pyrrolidin-2-yl)pyridin-3-yl)benzoic acid

### Synthesis of 4-[(1S)-2,2-dimethylcyclopropyl]aniline and 4-[(1R)-2,2-dimethylcyclopropyl]aniline

### Step 1: 4-methyl-N′-(propan-2-ylidene)benzenesulfonohydrazide

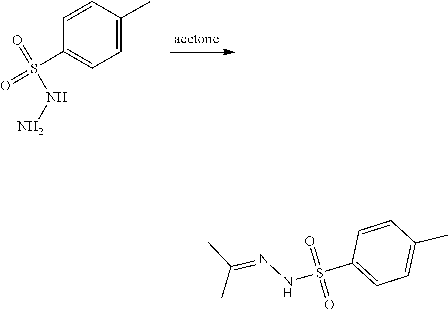

A solution of 4-toluenesulfonyl hydrazide (2.0 g, 10.7 mmol) in acetone (20 mL) was stirred at room temperature for 1 hour. Upon completion, the resulting mixture was concentrated under reduced pressure to give 4-methyl-N′-(propan-2-ylidene)benzenesulfonohydrazide (2.2 g, crude) as a yellow solid which was used in the next step without further purification. LCMS (ESI) [M+H]⁺: 227.

### Step 2: 4-(2,2-dimethylcyclopropyl)aniline

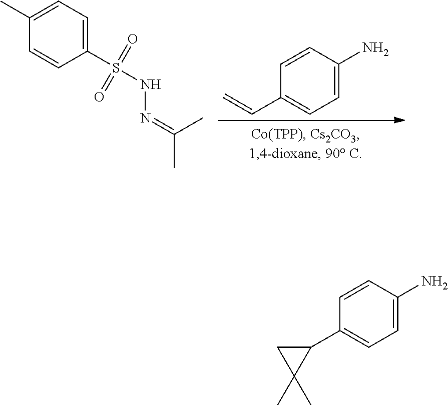

A solution of 4-methyl-N′-(propan-2-ylidene)benzenesulfonohydrazide (2.0 g, 8.8 mmol), 4-vinylaniline (2.1 g, 17.6 mmol), (tetraphenylporphinato)cobalt (300 mg, 0.4 mmol) and Cs₂CO₃ (4.3 g, 13.2 mmol) in 1,4-dioxane (20 mL) was stirred at 90° C. for 12 hours under nitrogen atmosphere. Upon completion, the resulting mixture was cooled to room temperature and concentrated under reduced pressure. The crude residue was purified by silica gel column chromatography, eluted with PE/EA (1:1) to afford 4-(2,2-dimethylcyclopropyl)aniline (300 mg, 21.0% yield) as a white solid. LCMS (ESI) [M+H]⁺: 162.

### Step 3: 4-[(1S)-2,2-dimethylcyclopropyl]aniline and 4-[(1R)-2,2-dimethylcyclopropyl]aniline

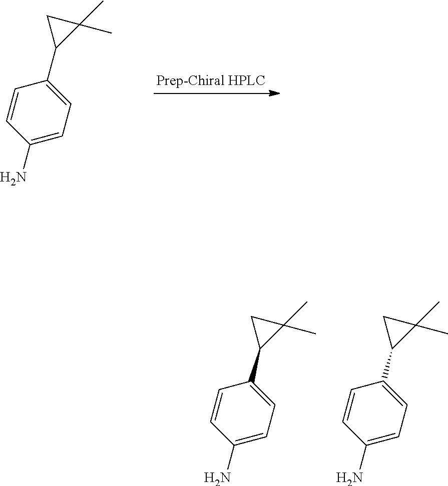

4-(2,2-dimethylcyclopropyl)aniline (4.0 g) was separated by Prep Chiral HPLC (conditions: Column: CHIRALPAK-IK, 3*25 mm, 5 μm; Mobile Phase A: Hex (0.1% DEA), Mobile Phase B: IPA; Flow rate: 70 mL/min; Gradient (B %): isocratic 2% B) to yield (S)-4-(2,2-dimethylcyclopropyl)aniline (Peak 1, 1.4 g, 36% yield) and (R)-4-(2,2-dimethylcyclopropyl)aniline (Peak 2, 1.2 g, 30% yield). LCMS (ESI) [M+H]⁺: 162

### Step 4: methyl 4-(6-(pyrrolidin-2-yl)pyridin-3-yl)benzoate

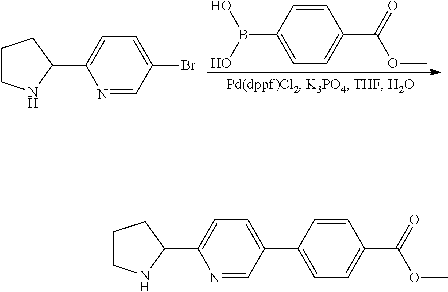

A solution of 5-bromo-2-(pyrrolidin-2-yl)pyridine (400 mg, 1.76 mmol), 4-(methoxycarbonyl)phenylboronic acid (380 mg, 2.1 mmol), Pd(dppf)Cl₂ (129 mg, 0.18 mmol) and K₃PO₄ (1.12 g, 5.28 mmol) in THF (4 mL) and H₂O (0.4 mL) was stirred at 80° C. for 1 hour under nitrogen atmosphere. Upon completion, the resulting mixture was concentrated under reduced pressure. The crude residue was purified by silica gel column chromatography, eluted with DCM/MeOH (3/1) to afford methyl 4-(6-(pyrrolidin-2-yl)pyridin-3-yl)benzoate (350 mg, 70% yield) as a yellow solid. LCMS (ESI) [M+H]⁺: 283.1

### Step 5: methyl 4-(6-(1-((4-((S)-2,2-dimethylcyclopropyl)phenyl)carbamoyl)pyrrolidin-2-yl)pyridin-3-yl)benzoate

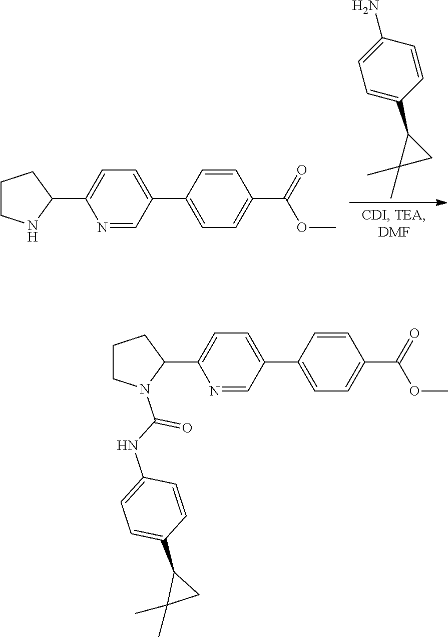 [SMILES: COC(=O)c1ccc(-c2ccc(C3CCCN3)nc2)cc1 ; CC1(C)C[C@@H]1c1ccc(N)cc1 ; COC(=O)c1ccc(-c2ccc(C3CCCN3C(=O)Nc3ccc([C@H]4CC4(C)C)cc3)nc2)cc1]

A solution of methyl 4-(6-(pyrrolidin-2-yl)pyridin-3-yl)benzoate (150 mg, 0.53 mmol), 4-[(1S)-2,2-dimethylcyclopropyl]aniline (Peak 1, 343 mg, 2.12 mmol), CDI (345 mg, 2.12 mmol) and triethylamine (215 mg, 2.12 mmol) in DMF (2 mL) was stirred at 40° C. for 1 hour. Upon completion, the resulting mixture cooled to room temperature and diluted with EtOAc (30 mL). The organic layer was washed with H₂O (10 mL), dried over anhydrous Na₂SO₄, filtered and concentrated under reduced pressure. The crude residue was purified by silica gel column chromatography, eluted with DCM/MeOH (10/1) to afford methyl 4-(6-(1-((4-((S)-2,2-dimethylcyclopropyl)phenyl)carbamoyl)pyrrolidin-2-yl)pyridin-3-yl)benzoate (100 mg, 40% yield) as a yellow solid. LCMS (ESI) [M+H]⁺: 470.2

### Step 6: 4-(6-(1-((4-((S)-2,2-dimethylcyclopropyl)phenyl)carbamoyl)pyrrolidin-2-yl)pyridin-3-yl)benzoic acid

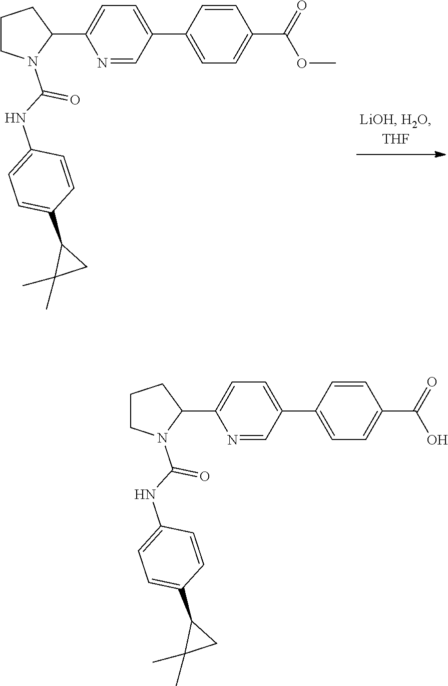 [SMILES: COC(=O)c1ccc(-c2ccc(C3CCCN3C(=O)Nc3ccc([C@H]4CC4(C)C)cc3)nc2)cc1 ; CC1(C)C[C@@H]1c1ccc(NC(=O)N2CCCC2c2ccc(-c3ccc(C(=O)O)cc3)cn2)cc1]

A solution of methyl 4-(6-(1-((4-((S)-2,2-dimethylcyclopropyl)phenyl)carbamoyl)pyrrolidin-2-yl)pyridin-3-yl)benzoate (100 mg, 0.21 mmol) and LiOH monohydrate (10.2 mg, 0.43 mmol) in THF (1 mL) and H₂O (1 mL) was stirred at room temperature for 1 hour. Upon completion, the mixture was acidified to pH 2 with HCl (2 M). The resulting mixture was extracted with EtOAc (3×10 mL). The combined organic layers were washed with H₂O (10 mL), dried over anhydrous Na₂SO₄, filtered and concentrated under reduced pressure. The crude residue was purified by reversed-phase flash chromatography (conditions: column, C18 silica gel; Mobile Phase A: Water (0.1% FA), Mobile Phase B: ACN; Gradient (B %): 10% to 50% in 10 min; detector, UV 254 nm) to yield 4-(6-(1-((4-((S)-2,2-dimethylcyclopropyl)phenyl)carbamoyl)pyrrolidin-2-yl)pyridin-3-yl)benzoic acid (50 mg, 51% yield) as a white solid. LCMS (ESI) [M+H]⁺: 456.2

### Step 7: 4-(6-((S)-1-((4-((S)-2,2-dimethylcyclopropyl)phenyl)carbamoyl)pyrrolidin-2-yl)pyridin-3-yl)benzoic acid and 4-(6-((R)-1-((4-((S)-2,2-dimethylcyclopropyl)phenyl)carbamoyl)pyrrolidin-2-yl)pyridin-3-yl)benzoic acid

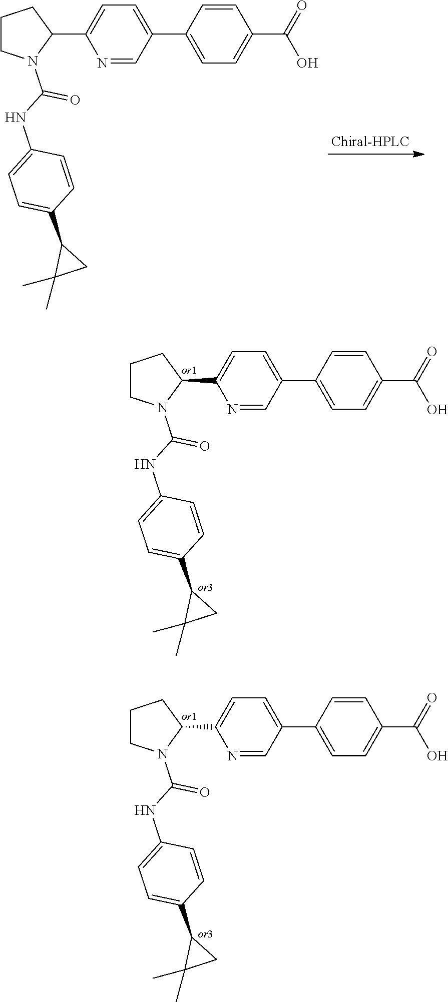

4-(6-(1-((4-((S)-2,2-dimethylcyclopropyl)phenyl)carbamoyl)pyrrolidin-2-yl)pyridin-3-yl)benzoic acid (40 mg, 0.088 mmol) was separated by prep-Chiral-HPLC (Column: CHIRALPAK IK 3*25 cm, 5 μm; Mobile Phase A: Hex (0.1% FA), Mobile Phase B: EtOH; Flow rate: 40 mL/min; Gradient (B %): isocratic 30% B; Wave Length: 250/260 nm; RT1 (min): 9.2; RT2 (min): 14.2) to afford (Compound 20a) 4-(6-((S)-1-((4-((S)-2,2-dimethylcyclopropyl)phenyl)carbamoyl)pyrrolidin-2-yl)pyridin-3-yl)benzoic acid (1^{st} peak, 14.7 mg, 36% yield; Method G, Peak 1, 1.10 min) and (Compound 20b) 4-(6-((R)-1-((4-((S)-2,2-dimethylcyclopropyl)phenyl)carbamoyl)pyrrolidin-2-yl)pyridin-3-yl)benzoic acid (2^{nd} peak, 13.7 mg, 34% yield; Method G, Peak 2, 2.60 min).

### Step 8: methyl 4-(6-(1-((4-((R)-2,2-dimethylcyclopropyl)phenyl)carbamoyl)pyrrolidin-2-yl)pyridin-3-yl)benzoate

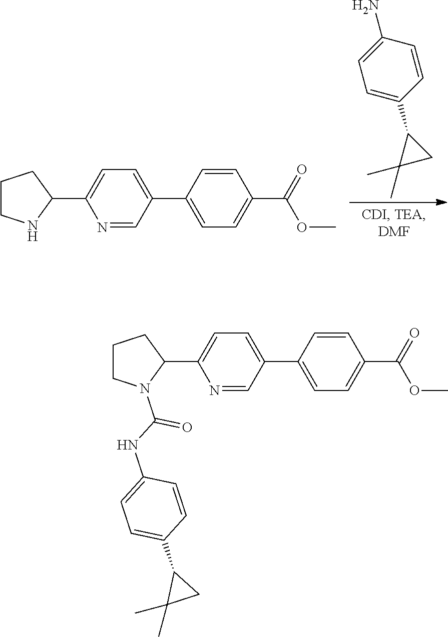 [SMILES: COC(=O)c1ccc(-c2ccc(C3CCCN3)nc2)cc1 ; CC1(C)C[C@H]1c1ccc(N)cc1 ; COC(=O)c1ccc(-c2ccc(C3CCCN3C(=O)Nc3ccc([C@@H]4CC4(C)C)cc3)nc2)cc1]

A solution of methyl 4-(6-pyrrolidin-2-yl)pyridin-3-yl)benzoate (150 mg, 0.53 mmol), 4-[(1R)-2,2-dimethylcyclopropyl]aniline (Peak 2, 343 mg, 2.12 mmol), CDI (345 mg, 2.12 mmol) and triethylamine (215 mg, 2.12 mmol) in DMF (2 mL) was stirred at 40° C. for 1 hour. Upon completion, the resulting mixture was diluted with EtOAc (30 mL). The organic layer was washed with H₂O (10 mL), dried over anhydrous Na₂SO₄, filtered and concentrated under reduced pressure. The crude residue was purified by silica gel column chromatography, eluted with DCM/MeOH (10/1) to afford methyl 4-(6-(1-((4-((R)-2,2-dimethylcyclopropyl)phenyl)carbamoyl)pyrrolidin-2-yl)pyridin-3-yl)benzoate (100 mg, 40% yield) as a yellow solid. LCMS (ESI) [M+H]⁺: 470.2

### Step 9: 4-(6-(1-((4-((R)-2,2-dimethylcyclopropyl)phenyl)carbamoyl)pyrrolidin-2-yl)pyridin-3-yl)benzoic acid

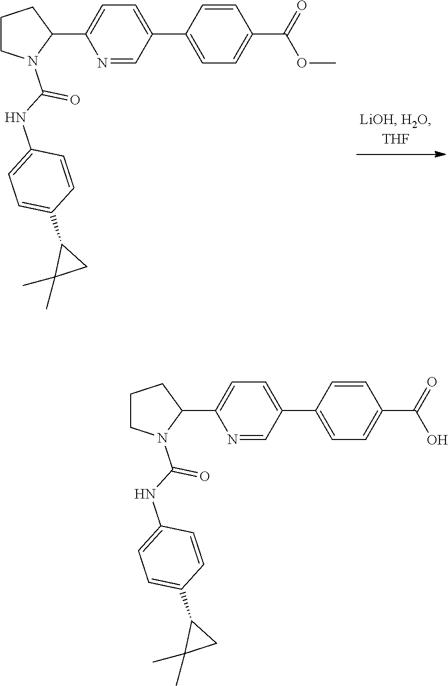 [SMILES: COC(=O)c1ccc(-c2ccc(C3CCCN3C(=O)Nc3ccc([C@@H]4CC4(C)C)cc3)nc2)cc1 ; CC1(C)C[C@H]1c1ccc(NC(=O)N2CCCC2c2ccc(-c3ccc(C(=O)O)cc3)cn2)cc1]

A solution of methyl 4-(6-(1-((4-((R)-2,2-dimethylcyclopropyl)phenyl)carbamoyl)pyrrolidin-2-yl)pyridin-3-yl)benzoate (100 mg, 0.21 mmol) and LiOH monohydrate (10.2 mg, 0.43 mmol) in THF (1 mL) and H₂O (1 mL) was stirred at room temperature for 1 hour. The mixture was acidified to pH 2 with HCl (2 M). The resulting mixture was extracted with EtOAc (3×10 mL). The combined organic layers were washed with H₂O (10 mL), dried over anhydrous Na₂SO₄, filtered and concentrated under reduced pressure. The crude residue was purified by reversed-phase flash chromatography (conditions: column, C18 silica gel; Mobile Phase A: Water (0.1% FA), Mobile Phase B: MeCN; Gradient (B %): 10% to 50% in 10 min; detector, UV 254 nm) to give 4-(6-(1-((4-((R)-2,2-dimethylcyclopropyl)phenyl)carbamoyl)pyrrolidin-2-yl)pyridin-3-yl)benzoic acid (50 mg, 51% yield) as a white solid. LCMS (ESI) [M+H]⁺: 456.2

### Step 10: 4-(6-((S)-1-((4-((R)-2,2-dimethylcyclopropyl)phenyl)carbamoyl)pyrrolidin-2-yl)pyridin-3-yl)benzoic acid and 4-(6-((R)-1-((4-((R)-2,2-dimethylcyclopropyl)phenyl)carbamoyl)pyrrolidin-2-yl)pyridin-3-yl)benzoic acid

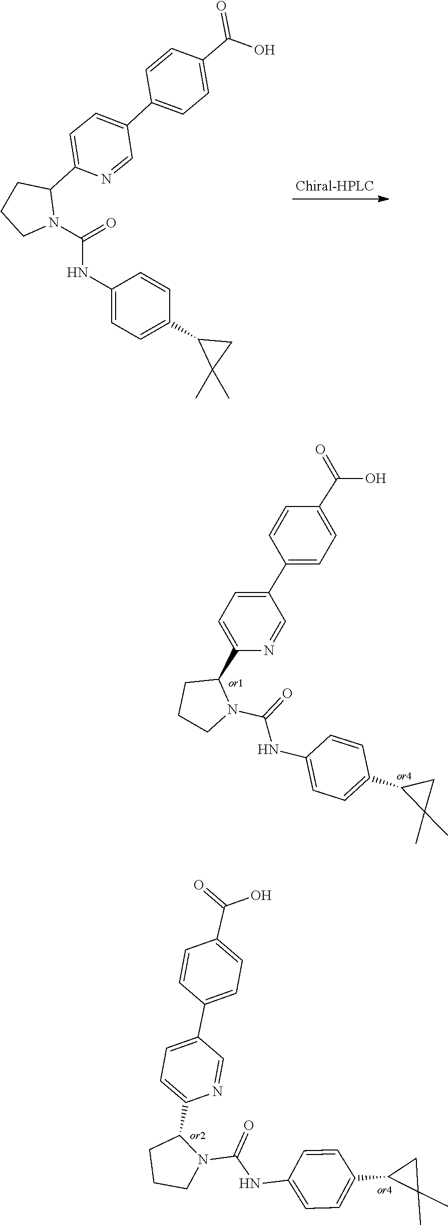 [SMILES: CC1(C)C[C@H]1c1ccc(NC(=O)N2CCCC2c2ccc(-c3ccc(C(=O)O)cc3)cn2)cc1 ; CC1(C)C[C@H]1c1ccc(NC(=O)N2CCC[C@H]2c2ccc(-c3ccc(C(=O)O)cc3)cn2)cc1 ; CC1(C)C[C@H]1c1ccc(NC(=O)N2CCC[C@@H]2c2ccc(-c3ccc(C(=O)O)cc3)cn2)cc1]

4-(6-(1-((4-((R)-2,2-dimethylcyclopropyl)phenyl)carbamoyl)pyrrolidin-2-yl)pyridin-3-yl)benzoic acid (40 mg, 0.088 mmol) was separated by prep-Chiral-HPLC (Column: CHIRALPAK IK 3*25 cm, 5 μm; Mobile Phase A: Hex (0.1% FA), Mobile Phase B: EtOH; Flow rate: 40 mL/min; Gradient (B %): isocratic 30% B; Wave Length: 250/260 nm; RT1 (min): 9.2; RT2 (min): 20) to afford (Compound 20c) 4-(6-((S)-1-((4-((R)-2,2-dimethylcyclopropyl)phenyl)carbamoyl)pyrrolidin-2-yl)pyridin-3-yl)benzoic acid (1^{st} peak, 16.4 mg, 41% yield; Method G, Peak 1, 1.08 min) and (Compound 20d) 4-(6-((R)-1-((4-((R)-2,2-dimethylcyclopropyl)phenyl)carbamoyl)pyrrolidin-2-yl)pyridin-3-yl)benzoic acid (2^{nd} peak, 15.8 mg, 39% yield; Method G, Peak 2, 2.20 min).

| Cmpd # | Characterization Data |
|---|---|
| 20a | LCMS (ESI) [M + H]⁺: 456.2 |
| | ¹H NMR (400 MHz, Methanol-d₄) δ 8.80 (d, J = 2.3 Hz, 1H), 8.17-8.06 (m, 3H), 7.77 (d, J = 8.2 |
| | Hz, 2H), 7.46 (d, J = 8.2 Hz, 1H), 7.25 (d, J = 8.6 Hz, 2H), 7.03 (d, J = 8.4 Hz, 2H), 5.21 (dd, J = |
| | 8.1, 3.0 Hz, 1H), 3.93-3.79 (m, 1H), 3.76-3.62 (m, 1H), 2.53-2.36 (m, 1H), 2.14-2.03 (m, |
| | 3H), 1.80 (dd, J = 8.5, 5.9 Hz, 1H), 1.19 (s, 3H), 0.79-0.66 (m, 5H). |
| 20b | LCMS (ESI) [M + H]⁺: 456.2 |
| | ¹H NMR (400 MHz, Methanol-d₄) δ 8.81 (d, J = 2.3 Hz, 1H), 8.18-8.06 (m, 3H), 7.77 (d, J = 8.4 |
| | Hz, 2H), 7.46 (d, J = 8.2 Hz, 1H), 7.25 (d, J = 8.5 Hz, 2H), 7.03 (d, J = 8.5 Hz, 2H), 5.21 (dd, J = |
| | 8.0, 2.9 Hz, 1H), 3.93-3.79 (m, 1H), 3.75-3.64 (m, 1H), 2.52-2.38 (m, 1H), 2.13-2.03 (m, |
| | 3H), 1.80 (dd, J = 8.5, 5.9 Hz, 1H), 1.19 (s, 3H), 0.78-0.67 (m, 5H). |
| 20c | LCMS (ESI) [M + H]⁺: 456.2 |
| | ¹H NMR (400 MHz, Methanol-d₄) δ 8.80 (d, J = 2.3 Hz, 1H), 8.17-8.03 (m, 3H), 7.77 (d, J = 8.3 |
| | Hz, 2H), 7.46 (d, J = 8.2 Hz, 1H), 7.25 (d, J = 8.5 Hz, 2H), 7.03 (d, J = 8.4 Hz, 2H), 5.21 (dd, J = |
| | 8.0, 2.9 Hz, 1H), 3.86 (dt, J = 11.7, 5.9 Hz, 1H), 3.75-3.64 (m, 1H), 2.54-2.38 (m, 1H), 2.14- |
| | 2.03 (m, 3H), 1.80 (dd, J = 8.5, 5.9 Hz, 1H), 1.19 (s, 3H), 0.80-0.66 (m, 5H). |
| 20d | LCMS (ESI) [M + H]⁺: 456.3 |
| | ¹H NMR (400 MHz, Methanol-d₄) δ 8.80 (d, J = 2.3 Hz, 1H), 8.16-8.05 (m, 3H), 7.76 (d, J = 8.4 |
| | Hz, 2H), 7.46 (d, J = 8.2 Hz, 1H), 7.25 (d, J = 8.5 Hz, 2H), 7.03 (d, J = 8.5 Hz, 2H), 5.21 (dd, J = |
| | 8.1, 3.0 Hz, 1H), 3.86 (dt, J = 11.7, 5.9 Hz, 1H), 3.69 (dt, J = 9.4, 7.2 Hz, 1H), 2.52-2.36 (m, 1H), |
| | 2.12-2.01 (m, 3H), 1.80 (dd, J = 8.5, 5.8 Hz, 1H), 1.19 (s, 3H), 0.80-0.63 (m, 5H). |

### Example 22: (R)-3-fluoro-4′-(2-(3-(4-isopropylphenyl)ureido)-3-methylbutanamido)-[1,1′-biphenyl]-4-carboxylic acid

### Step 1: tert-butyl (R)-4′-(2-((tert-butoxycarbonyl)amino)-3-methylbutanamido)-3-fluoro-[1,1′-biphenyl]-4-carboxylate

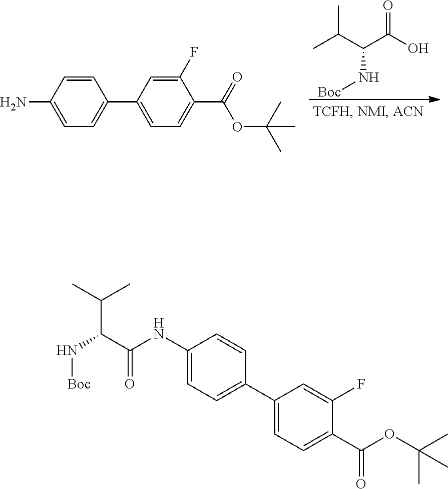 [SMILES: CC(C)(C)OC(=O)c1ccc(-c2ccc(N)cc2)cc1F ; CC(C)[C@@H](NC(=O)OC(C)(C)C)C(=O)O ; CC(C)[C@@H](NC(=O)OC(C)(C)C)C(=O)Nc1ccc(-c2ccc(C(=O)OC(C)(C)C)c(F)c2)cc1]

A solution of tert-butyl 4′-amino-3-fluoro-[1,1′-biphenyl]-4-carboxylate (150 mg, 0.5 mmol), (tert-butoxycarbonyl)-D-valine (170 mg, 0.8 mmol), 1-methyl-1H-imidazole (129 mg, 1.5 mmol) and N-(chloro(dimethylamino)methylene)-N-methylmethanaminium hexafluorophosphate(V) (220 mg, 0.8 mmol) in ACN (6 mL) was stirred at room temperature for 2 hours. Upon completion, the resulting mixture was concentrated under reduced pressure. The crude residue was purified by silica gel column chromatography, eluted with PE/EA (1:1) to afford tert-butyl (R)-4′-(2-((tert-butoxycarbonyl)amino)-3-methylbutanamido)-3-fluoro-[1,1′-biphenyl]-4-carboxylate (120 mg, 47% yield) as a light yellow solid. LCMS [M+H]⁺: 487

### Step 2: (R)-4′-(2-amino-3-methylbutanamido)-3-fluoro-[1,1′-biphenyl]-4-carboxylic acid

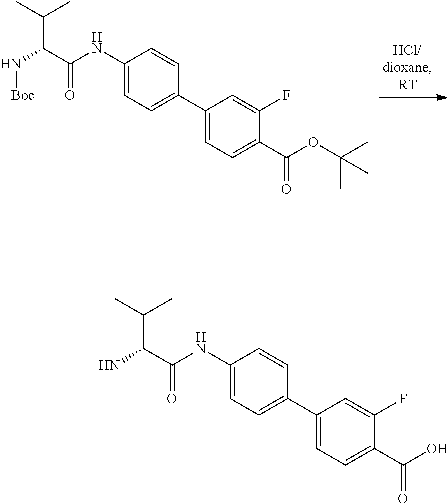 [SMILES: CC(C)[C@@H](NC(=O)OC(C)(C)C)C(=O)Nc1ccc(-c2ccc(C(=O)OC(C)(C)C)c(F)c2)cc1 ; CC(C)[C@@H](N)C(=O)Nc1ccc(-c2ccc(C(=O)O)c(F)c2)cc1]

To a mixture of tert-butyl (R)-4′-(2-((tert-butoxycarbonyl)amino)-3-methylbutanamido)-3-fluoro-[1,1′-biphenyl]-4-carboxylate (115 mg, 0.2 mmol) in dioxane (1 mL) was added HCl (20 mmol, 5 mL, 4 M in dioxane). The resulting mixture was stirred at room temperature for 1 hour. Upon completion, the resulting mixture was concentrated under reduced pressure to give (R)-4′-(2-amino-3-methylbutanamido)-3-fluoro-[1,1′-biphenyl]-4-carboxylic acid (100 mg, crude) as a yellow oil, which was used in the next step without further purification. LCMS [M+H]⁺: 331

### Step 3: (R)-3-fluoro-4′-(2-(3-(4-isopropylphenyl)ureido)-3-methylbutanamido)-[1,1′-biphenyl]-4-carboxylic acid

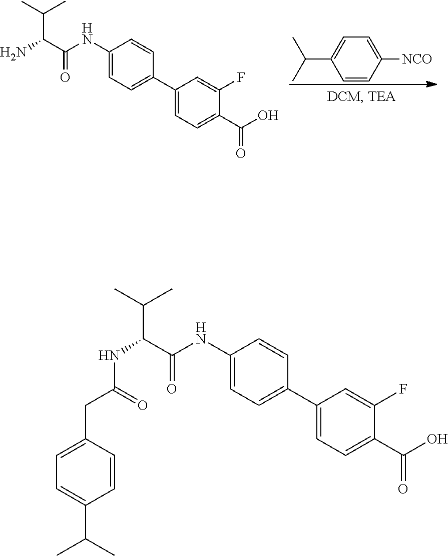

To a mixture of (R)-4′-(2-amino-3-methylbutanamido)-3-fluoro-[1,1′-biphenyl]-4-carboxylic acid (50 mg, 0.1 mmol) in DCM (2 mL) were added triethylamine (45 mg, 0.4 mmol) and 1-isocyanato-4-isopropylbenzene (24 mg, 0.1 mmol) under nitrogen atmosphere. The resulting mixture was stirred at room temperature for 2 hours. Upon completion, the reaction was quenched with ice water at 0° C. The resulting mixture was extracted with EtOAc (3×5 mL). The combined organic layers were dried over anhydrous Na₂SO₄, filtered and concentrated under reduced pressure. The crude residue was purified by Prep-HPLC (conditions: Column: Xbridge Phenyl OBD Column, 30*150 mm 5 μm; Mobile Phase A: Water (0.1% FA), Mobile Phase B: ACN; Flow rate: 60 mL/min; Gradient (B %): 38% B to 58% B in 15 min; Wave Length: 254 nm/220 nm; RT1 (min): 9.18) to afford (R)-3-fluoro-4′-(2-(3-(4-isopropylphenyl)ureido)-3-methylbutanamido)-[1,1′-biphenyl]-4-carboxylic acid (25.9 mg, Compound 22; Method E, Peak 1, 1.40 min—analyzed against Compound 23) as a white solid.

| Cmpd # | Characterization Data |
|---|---|
| 22 | LCMS (ESI) [M + H]⁺: 492.2 |
| | ¹H NMR (400 MHz, DMSO-d₆) δ 13.18 (bs, 1H), 10.39 (s, 1H), 8.61 (s, 1H), 7.93-7.91 (m, 1H), |
| | 7.75 (s, 4H), 7.62-7.59 (m, 2H), 7.30-7.27 (m, 2H), 7.10 (d, J = 8.4 Hz, 2H), 6.47 (d, J = 8.4 |
| | Hz, 1H), 4.33-4.29 (m, 1H), 2.83-2.76 (m, 1H), 2.08-2.04 (m, 1H), 1.23 (d, J = 6.8 Hz, 6H), |
| | 0.97-0.95 (m, 6H). |

### Example 23: (S)-3-fluoro-4′-(2-(3-(4-isopropylphenyl)ureido)-3-methylbutanamido)-[1,1′-biphenyl]-4-carboxylic acid

### Step 1: tert-butyl (S)-4′-(2-((tert-butoxycarbonyl)amino)-3-methylbutanamido)-3-fluoro-[1,1′-biphenyl]-4-carboxylate

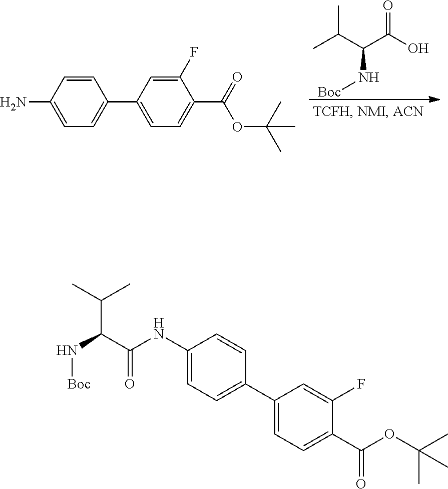 [SMILES: CC(C)(C)OC(=O)c1ccc(-c2ccc(N)cc2)cc1F ; CC(C)[C@H](NC(=O)OC(C)(C)C)C(=O)O ; CC(C)[C@H](NC(=O)OC(C)(C)C)C(=O)Nc1ccc(-c2ccc(C(=O)OC(C)(C)C)c(F)c2)cc1]

A solution of tert-butyl 4′-amino-3-fluoro-[1,1′-biphenyl]-4-carboxylate (150 mg, 0.5 mmol), (tert-butoxycarbonyl)-L-valine (170 mg, 0.8 mmol), 1-methyl-1H-imidazole (129 mg, 1.5 mmol) and N-(chloro(dimethylamino)methylene)-N-methylmethanaminium hexafluorophosphate(V) (220 mg, 0.8 mmol) in ACN (6 mL) was stirred at room temperature for 2 hours. Upon completion, the resulting mixture was concentrated under reduced pressure. The crude residue was purified by silica gel column chromatography, eluted with PE/EA (1:1) to afford tert-butyl (S)-4′-(2-((tert-butoxycarbonyl)amino)-3-methylbutanamido)-3-fluoro-[1,1′-biphenyl]-4-carboxylate (125 mg, 49% yield) as a light yellow solid. LCMS [M+H]⁺: 487

### Step 2: (S)-4′-(2-amino-3-methylbutanamido)-3-fluoro-[1,1′-biphenyl]-4-carboxylic acid

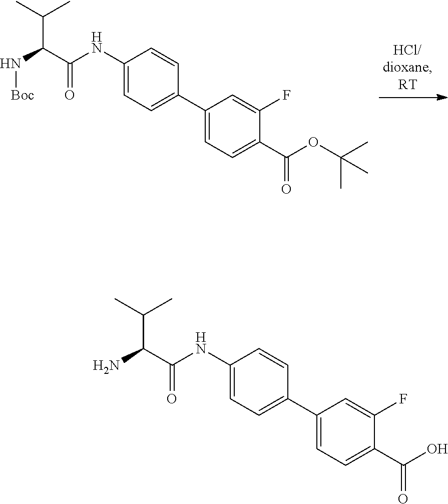 [SMILES: CC(C)[C@H](NC(=O)OC(C)(C)C)C(=O)Nc1ccc(-c2ccc(C(=O)OC(C)(C)C)c(F)c2)cc1 ; CC(C)[C@H](N)C(=O)Nc1ccc(-c2ccc(C(=O)O)c(F)c2)cc1]

To a mixture of tert-butyl (S)-4′-(2-((tert-butoxycarbonyl)amino)-3-methylbutanamido)-3-fluoro-[1,1′-biphenyl]-4-carboxylate (120 mg, 0.2 mmol) in dioxane (1 mL) was added HCl (20 mmol, 5 mL, 4 M in dioxane). The resulting mixture was stirred at room temperature for 1 hour. Upon completion, the resulting mixture was concentrated under reduced pressure to yield (S)-4′-(2-amino-3-methylbutanamido)-3-fluoro-[1,1′-biphenyl]-4-carboxylic acid (110 mg, crude) as a yellow oil, which was used in the next step without further purification. LCMS [M+H]⁺: 331

### Step 3: (S)-3-fluoro-4′-(2-(3-(4-isopropylphenyl)ureido)-3-methylbutanamido)-[1,1′-biphenyl]-4-carboxylic acid

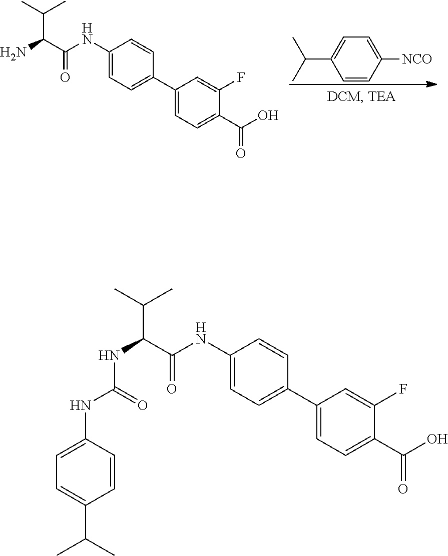

To a mixture of (S)-4′-(2-amino-3-methylbutanamido)-3-fluoro-[1,1′-biphenyl]-4-carboxylic acid (100 mg, 0.3 mmol) in DCM (4.5 mL) were added triethylamine (92 mg, 0.9 mmol) and 1-isocyanato-4-isopropylbenzene (49 mg, 0.3 mmol). The resulting mixture was stirred at room temperature for 2 hours. Upon completion, the reaction was quenched with ice water at 0° C. The resulting mixture was extracted with EtOAc (3×8 mL). The combined organic layers were dried over anhydrous Na₂SO₄, filtered and concentrated under reduced pressure. The crude residue was purified by Prep-HPLC (conditions: Column: Xbridge Phenyl OBD Column, 30*150 mm 5 μm; Mobile Phase A: Water (0.1% FA), Mobile Phase B: ACN; Flow rate: 60 mL/min; Gradient (B %): 38% B to 58% B in 15 min; Wave Length: 254 nm/220 nm; RT1 (min): 9.22) to afford (S)-3-fluoro-4′-(2-(3-(4-isopropylphenyl)ureido)-3-methylbutanamido)-[1,1′-biphenyl]-4-carboxylic acid (22.8 mg, Compound 23; Method E, Peak 2, 1.59 min—analyzed against Compound 22) as a white solid.

| Cmpd # | Characterization Data |
|---|---|
| 23 | LCMS (ESI) [M + H]⁺: 492.3 |
| | ¹H NMR (400 MHz, DMSO-d₆) δ 10.31 (s, 1H), 8.67 (s, 1H), 7.76-7.66 (m, 5H), 7.48-7.43 (m, |
| | 2H), 7.31 (d, J = 6.8 Hz, 2H), 7.10 (d, J = 8.4 Hz, 2H), 6.55 (d, J = 9.2 Hz, 1H), 4.32-4.29 (m, |
| | 1H), 2.83-2.79 (m, 1H), 2.07-2.03 (m, 1H), 1.19 (d, J = 10.8 Hz, 6H), 0.96-0.91 (m, 6H). |

### Example 25: 4′-(2-((4-isopropylphenyl)carbamoyl)pyrazolidine-1-carboxamido)-[1,1′-biphenyl]-4-carboxylic acid

### Step 1: tert-butyl 2-((4′-(tert-butoxycarbonyl)-[1,1′-biphenyl]-4-yl)carbamoyl)pyrazolidine-1-carboxylat

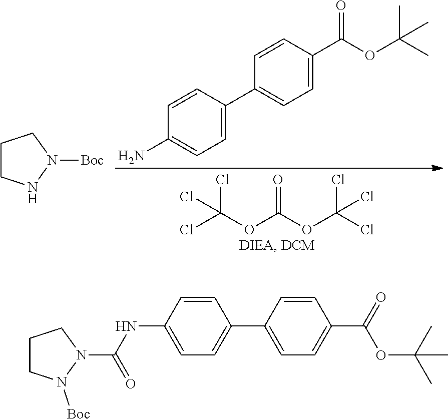

To a mixture of bis(trichloromethyl) carbonate (68 mg, 0.2 mmol) in DCM (6 mL) were added tert-butyl pyrazolidine-1-carboxylate (100 mg, 0.5 mmol) and DIEA (225 mg, 1.7 mmol) at 0° C. After 30 minutes, tert-butyl pyrazolidine-1-carboxylate (312 mg, 1.1 mmol) was added in portions. The resulting mixture was warmed to room temperature and stirred for an additional 30 minutes. Upon completion, the reaction was quenched with water at 0° C. The resulting mixture was extracted with EtOAc (3×10 mL). The combined organic layers were dried over anhydrous Na₂SO₄, filtered and concentrated under reduced pressure. The crude residue was purified by reversed-phase flash chromatography (conditions: column, C18 silica gel; mobile phase A: Water (10 mmol/L NH₄HCO₃), Mobile Phase B: MeCN; Gradient (B %): 5% to 95% gradient in 25 min; detector, UV 200 nm) to give tert-butyl 2-((4′-(tert-butoxycarbonyl)-[1,1′-biphenyl]-4-yl)carbamoyl)pyrazolidine-1-carboxylate (110 mg, 40% yield) as an off-white solid. LCMS (ESI) [M+H]⁺: 468

### Step 2: 4′-(pyrazolidine-1-carboxamido)-[1,1′-biphenyl]-4-carboxylic acid

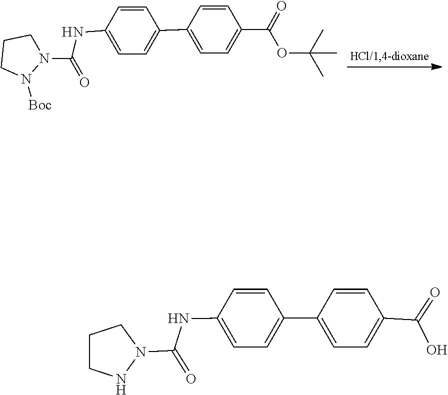 [SMILES: CC(C)(C)OC(=O)c1ccc(-c2ccc(NC(=O)N3CCCN3C(=O)OC(C)(C)C)cc2)cc1 ; O=C(O)c1ccc(-c2ccc(NC(=O)N3CCCN3)cc2)cc1]

To a mixture of tert-butyl 2-((4′-(tert-butoxycarbonyl)-[1,1′-biphenyl]-4-yl)carbamoyl)pyrazolidine-1-carboxylate (100 mg, 0.2 mmol) in dioxane (1 mL) was added HCl (20 mmol, 5 mL, 4 M in dioxane) dropwise at 0° C. The resulting mixture was warmed to room temperature and stirred for 12 hours. Upon completion, the resulting mixture was concentrated under reduced pressure to give 4′-(pyrazolidine-1-carboxamido)-[1,1′-biphenyl]-4-carboxylic acid (80 mg, crude) as a white solid. LCMS (ESI) [M+H]⁺: 312

### Step 3: 4′-(2-((4-isopropylphenyl)carbamoyl)pyrazolidine-1-carboxamido)-[1,1′-biphenyl]-4-carboxylic acid

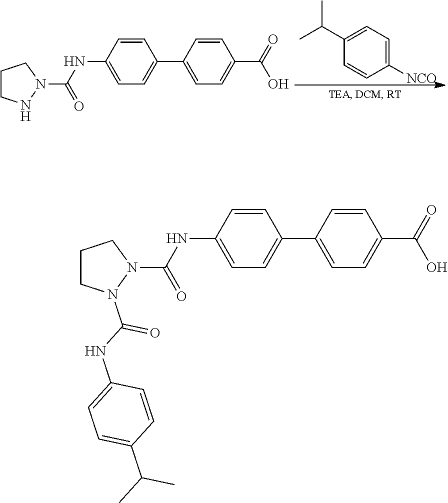

To a mixture of 4′-(pyrazolidine-1-carboxamido)-[1,1′-biphenyl]-4-carboxylic acid (78 mg, 0.2 mmol) in DCM (3.5 mL) were added triethylamine (45 mg, 0.4 mmol) and 1-isocyanato-4-isopropylbenzene (39 mg, 0.2 mmol) dropwise at 0° C. under nitrogen atmosphere. The resulting mixture was warmed to room temperature and stirred for 2 hours. Upon completion, the reaction was quenched with ice water at 0° C. The resulting mixture was extracted with EtOAc (3×8 mL). The combined organic layers were dried over anhydrous Na₂SO₄, filtered and concentrated under reduced pressure. The crude residue was purified by Prep-HPLC (conditions: Column: Xselect CSH Prep C18, 30*150 mm 5 μm; Mobile Phase A: Water (0.1% FA), Mobile Phase B: MeCN; Flow rate: 60 mL/min; Gradient (B %): 43% to 64% B in 12 min; Wave Length: 254 nm/220 nm; RT1 (min): 10.88) to afford 4′-(2-((4-isopropylphenyl)carbamoyl)pyrazolidine-1-carboxamido)-[1,1′-biphenyl]-4-carboxylic acid (26.3 mg, 24% yield, Compound 25) as a white solid.

| Cmpd # | Characterization Data |
|---|---|
| 25 | LCMS (ESI) [M + H]⁺: 473.3 |
| | ¹H NMR (400 MHz, DMSO-d₆) δ 12.90 (bs, 1H), 9.42 (s, 1H), 9.22 (s, 1H), 7.99 (d, J = 8.8 Hz, |
| | 2H), 7.83-7.75 (m, 4H), 7.70-7.65 (m, 2H), 7.57-7.52 (m, 2H), 7.15-7.10 (m, 2H), 4.17 (bs, |
| | 2H), 3.01 (bs, 2H), 2.86-2.77 (m, 1H), 2.02-1.91 (m, 2H), 1.17 (d, J = 6.9 Hz, 6H). |

### Example 29: (S)-3-(6-(1-((4-isopropylphenyl)carbamoyl)pyrrolidin-2-yl)pyridin-3-yl)benzoic acid & (R)-3-(6-(1-((4-isopropylphenyl)carbamoyl)pyrrolidin-2-yl)pyridin-3-yl)benzoic acid

### Step 1: methyl 3-(6-(1-((4-isopropylphenyl)carbamoyl)pyrrolidin-2-yl)pyridin-3-yl)benzoate

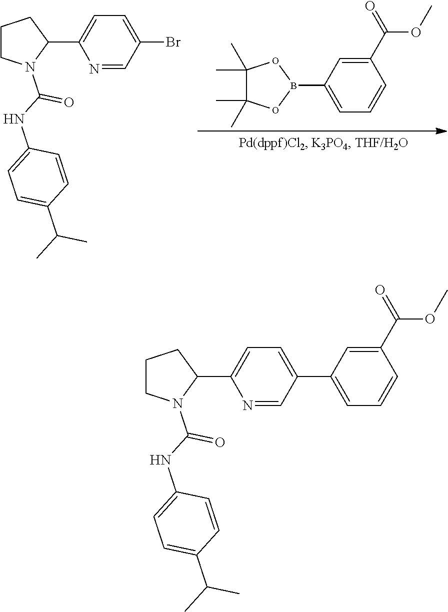

A solution of 2-(5-bromopyridin-2-yl)-N-(4-isopropylphenyl)pyrrolidine-1-carboxamide (100 mg, 0.26 mmol), methyl 3-(4,4,5,5-tetramethyl-1,3,2-dioxaborolan-2-yl)benzoate (87.8 mg, 0.34 mmol), Pd(dppf)Cl₂ (18.8 mg, 0.026 mmol), K₃PO₄ (164 mg, 0.77 mmol) in THF (2 mL) and H₂O (0.2 mL) was stirred at 80° C. for 2 hours under nitrogen atmosphere. Upon completion, the resulting mixture was concentrated under reduced pressure. The crude residue was directly purified by silica gel column chromatography, eluted with PE/EA (1:1) to afford methyl 3-(6-(1-((4-isopropylphenyl)carbamoyl)pyrrolidin-2-yl)pyridin-3-yl)benzoate (80 mg, 70% yield) as a light yellow oil. LCMS (ESI) [M+H]⁺: 444.2

### Step 2: 3-(6-(1-((4-isopropylphenyl)carbamoyl)pyrrolidin-2-yl)pyridin-3-yl)benzoic acid

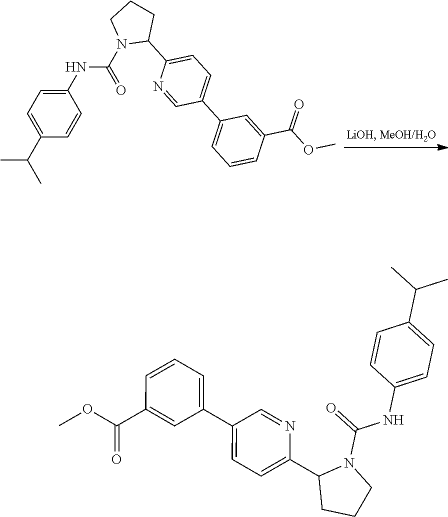

A solution of methyl 3-(6-(1-((4-isopropylphenyl)carbamoyl)pyrrolidin-2-yl)pyridin-3-yl)benzoate (75 mg, 0.163 mmol) and LiOH monohydrate (68.0 mg, 1.63 mmol) in MeOH (3 mL) and H₂O (2 mL) was stirred at room temperature for 2 hours. Upon completion, the mixture was acidified to pH=4 with conc. HCl acid and then concentrated under reduced pressure. The crude residue was directly purified by reversed-phase flash chromatography (conditions: column, C18 silica gel; mobile phase A: Water (0.1% FA), Mobile phase B: MeCN, Gradient (B %): 25% to 80% in 25 min; detector: UV 254 nm) to afford 3-(6-(1-((4-isopropylphenyl)carbamoyl)pyrrolidin-2-yl)pyridin-3-yl)benzoic acid (70 mg, 96% yield) as a white solid. LCMS (ESI) [M+H]⁺: 430.2

### Step 3: (S)-3-(6-(1-((4-isopropylphenyl)carbamoyl)pyrrolidin-2-yl)pyridin-3-yl)benzoic acid & (R)-3-(6-(1-((4-isopropylphenyl)carbamoyl)pyrrolidin-2-yl)pyridin-3-yl)benzoic acid

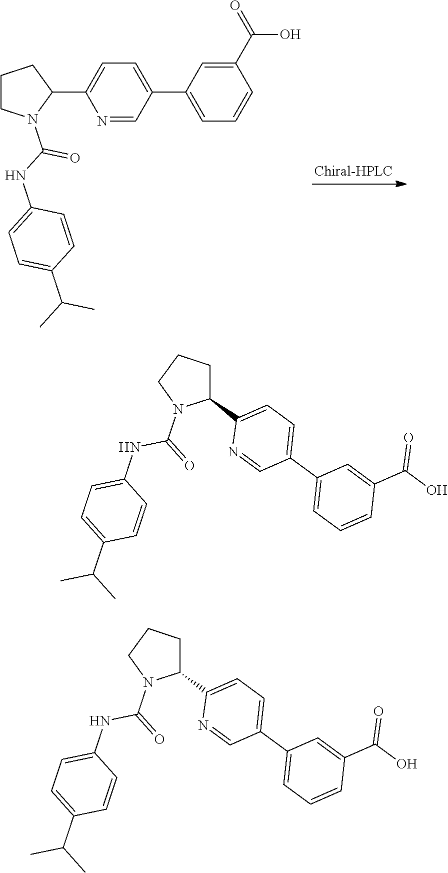

3-(6-(1-((4-isopropylphenyl)carbamoyl)pyrrolidin-2-yl)pyridin-3-yl)benzoic acid (70 mg) was separate by Prep-Chiral-HPLC (conditions: Column: CHIRAL ART Cellulose-SZ, 3*25 cm, m; Mobile Phase A: Hexane (0.1% FA), Mobile Phase B: EtOH; Flow rate: 40 mL/min; Gradient (B %): isocratic 30% B; Wave Length: 230/240 nm; RT1 (min): 8.1; RT2 (min): 11.4) to give (Compound 29a) (S)-3-(6-(1-((4-isopropylphenyl)carbamoyl)pyrrolidin-2-yl)pyridin-3-yl)benzoic acid (1^{st} peak, 18.0 mg, 26% yield; Method K, Peak 1, 1.00 min) as a white solid and (Compound 29b) (R)-3-(6-(1-((4-isopropylphenyl)carbamoyl)pyrrolidin-2-yl)pyridin-3-yl)benzoic acid (2^{nd} peak, 14.9 mg, 21 yield; Method K, Peak 2, 1.43 min) as a white solid.

| Cmpd # | Characterization Data |
|---|---|
| 29a | LCMS (ESI) [M + H]⁺: 430.2 |
| | ¹H NMR (400 MHz, DMSO-d₆) δ 13.14 (bs, 1H), 8.85 (d, J = 2.0 Hz, 1H), 8.21 (d, J = 14.2 Hz, |
| | 2H), 8.07 (dd, J = 8.2, 2.4 Hz, 1H), 8.00-7.92 (m, 2H), 7.62 (t, J = 7.8 Hz, 1H), 7.38-7.31 (m, |
| | 3H), 7.06 (d, J = 8.5 Hz, 2H), 5.19-5.11 (m, 1H), 3.79 (q, J = 5.2, 4.2 Hz, 1H), 3.66-3.53 (m, |
| | 1H), 2.79 (hept, J = 6.9 Hz, 1H), 2.37-2.22 (m, 1H), 2.05-1.89 (m, 3H), 1.15 (d, J = 6.9 Hz, 6H). |
| 29b | LCMS (ESI) [M + H]⁺: 430.2 |
| | ¹H NMR (400 MHz, DMSO-d₆) δ 13.16 (bs, 1H), 8.85 (d, J = 2.0 Hz, 1H), 8.21 (d, J = 12.7 Hz, |
| | 2H), 8.07 (dd, J = 8.2, 2.2 Hz, 1H), 8.02-7.93 (m, 2H), 7.62 (t, J = 7.8 Hz, 1H), 7.42-7.32 (m, |
| | 3H), 7.06 (d, J = 8.5 Hz, 2H), 5.21-5.07 (m, 1H), 3.85-3.70 (m, 1H), 3.59 (q, J = 7.9 Hz, 1H), |
| | 2.79 (hept, J = 6.9 Hz, 1H), 2.40-2.22 (m, 1H), 2.03-1.89 (m, 3H), 1.15 (d, J = 6.9 Hz, 6H). |

### Example 30: (S)-4-(6-(1-((4-isopropylphenyl)carbamoyl)-2-methylazetidin-2-yl)pyridin-3-yl)benzoic acid & (R)-4-(6-(1-((4-isopropylphenyl)carbamoyl)-2-methylazetidin-2-yl)pyridin-3-yl)benzoic acid

### Step 1: tert-butyl 2-(5-(4-(methoxycarbonyl)phenyl)pyridin-2-yl)-2-methylazetidine-1-carboxylate

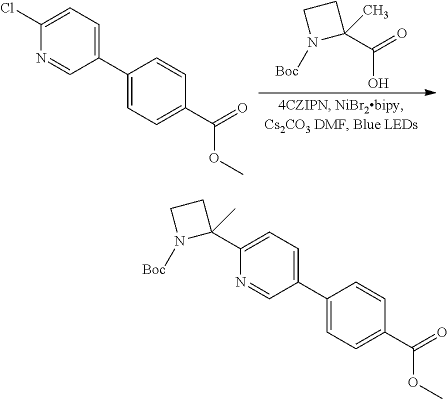

A solution of methyl 4-(6-chloropyridin-3-yl)benzoate (900 mg, 3.63 mmol), Cs₂CO₃ (1.78 g, 5.45 mmol), tetrakis(9H-carbazol-9-yl)benzene-1,4-dicarbonitrile (71.7 mg, 0.091 mmol) and NiBr₂·bipy (136 mg, 0.363 mmol) in DMF (10 mL) was treated with 1-(tert-butoxycarbonyl)-2-methylazetidine-2-carboxylic acid (1.17 g, 5.45 mmol) at room temperature under nitrogen atmosphere. The resulting mixture was stirred and irradiated with 450 nm blue LEDs for 24 hours. The precipitated solids were collected by filtration and the filter cake was washed with DMF (2×2 mL). The filtrate was concentrated under reduced pressure. The crude residue was purified by reversed-phase flash chromatography (conditions: column, C18 silica gel; mobile phase A: Water (0.1% FA), Mobile Phase B: MeCN; Gradient (B %): 22% to 50% in 16 min; detector: UV 254 nm) to afford tert-butyl 2-(5-(4-(methoxycarbonyl)phenyl)pyridin-2-yl)-2-methylazetidine-1-carboxylate (66 mg, 4.8% yield) as a greenish solid. LCMS (ESI) [M+H]⁺: 383.5

### Step 2: methyl 4-(6-(2-methylazetidin-2-yl)pyridin-3-yl)benzoate

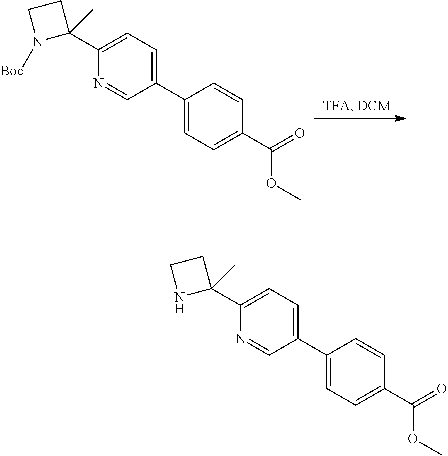 [SMILES: COC(=O)c1ccc(-c2ccc(C3(C)CCN3C(=O)OC(C)(C)C)nc2)cc1 ; COC(=O)c1ccc(-c2ccc(C3(C)CCN3)nc2)cc1]

A solution of tert-butyl 2-(5-(4-(methoxycarbonyl)phenyl)pyridin-2-yl)-2-methylazetidine-1-carboxylate (60 mg, 0.157 mmol) in DCM (2 mL) was treated with TFA (2 mL) at room temperature under nitrogen atmosphere. The resulting mixture was stirred for 1 hour. Upon completion, the resulting mixture was concentrated under reduced pressure. The crude residue was directly purified by reversed-phase flash chromatography (Conditions: column, C18 silica gel; mobile phase A: Water (0.1% FA), Mobile Phase B: MeCN; Gradient (B %): 20% to 30% in 10 min; detector: UV 254 nm) to afford methyl 4-(6-(2-methylazetidin-2-yl)pyridin-3-yl)benzoate (35 mg, 79% yield) as a white solid. LCMS (ESI) [M+H]⁺: 283.3

### Step 3: methyl 4-(6-(1-((4-isopropylphenyl)carbamoyl)-2-methylazetidin-2-yl)pyridin-3-yl)benzoate

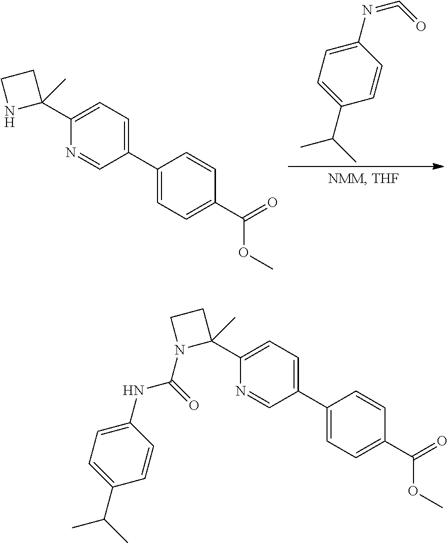

A solution of 1-isocyanato-4-isopropylbenzene (25.7 mg, 0.159 mmol) and methyl 4-(6-(2-methylazetidin-2-yl)pyridin-3-yl)benzoate (30 mg, 0.106 mmol) in THF (4 mL) was treated with NMM (32.2 mg, 0.318 mmol) at room temperature under nitrogen atmosphere. The resulting mixture was stirred for 1 hour. Upon completion, the resulting mixture was concentrated under reduced pressure. The crude residue was directly purified by reversed-phase flash chromatography (conditions: column, C18 silica gel; mobile phase A: Water (0.1% FA), Mobile Phase B: MeCN; Gradient (B %): 43% to 56% in 24 min; detector, UV 254 nm) to afford methyl 4-(6-(1-((4-isopropylphenyl)carbamoyl)-2-methylazetidin-2-yl)pyridin-3-yl)benzoate (30 mg, 64% yield) as a white solid. LCMS (ESI) [M+H]⁺: 444.6

### Step 4: 4-(6-(1-((4-isopropylphenyl)carbamoyl)-2-methylazetidin-2-yl)pyridin-3-yl)benzoic acid

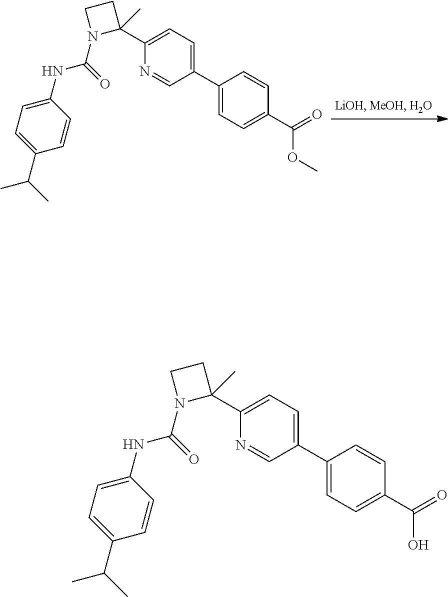

A solution of methyl 4-(6-(1-((4-isopropylphenyl)carbamoyl)-2-methylazetidin-2-yl)pyridin-3-yl)benzoate (30 mg, 0.068 mmol) and LiOH monohydrate (14.0 mg, 0.34 mmol) in MeOH (1 mL) and H₂O (1 mL) at room temperature under nitrogen atmosphere. The resulting mixture was stirred at 50° C. for 1 hour. Upon completion, the resulting mixture was concentrated under reduced pressure. The crude residue was directly purified by reversed-phase flash chromatography (conditions: column, C18 silica gel; mobile phase A: Water (0.1% FA), Mobile Phase B: MeCN; Gradient (B %): 26% to 39% in 14 min; detector: UV 254 nm) to afford 4-(6-(1-((4-isopropylphenyl)carbamoyl)-2-methylazetidin-2-yl)pyridin-3-yl)benzoic acid (15 mg, 52% yield) as a white solid. LCMS (ESI) [M+H]⁺: 430.5

### Step 5: (S)-4-(6-(1-((4-isopropylphenyl)carbamoyl)-2-methylazetidin-2-yl)pyridin-3-yl)benzoic acid & (R)-4-(6-(1-((4-isopropylphenyl)carbamoyl)-2-methylazetidin-2-yl)pyridin-3-yl)benzoic acid

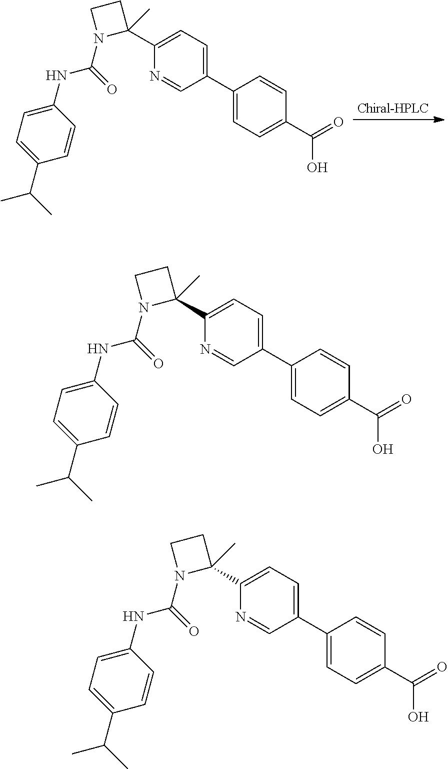 [SMILES: CC(C)c1ccc(NC(=O)N2CCC2(C)c2ccc(-c3ccc(C(=O)O)cc3)cn2)cc1 ; CC(C)c1ccc(NC(=O)N2CC[C@@]2(C)c2ccc(-c3ccc(C(=O)O)cc3)cn2)cc1 ; CC(C)c1ccc(NC(=O)N2CC[C@]2(C)c2ccc(-c3ccc(C(=O)O)cc3)cn2)cc1]

4-(6-(1-((4-isopropylphenyl)carbamoyl)-2-methylazetidin-2-yl)pyridin-3-yl)benzoic acid (14 mg, 0.033 mmol) was separated by prep Chiral-HPLC (conditions: Column: CHIRALPAK IK 3*25 cm, 5 μm; Mobile Phase A: Hexane (0.1% FA), Mobile Phase B: EtOH; Flow rate: 40 mL/min; Gradient (B %): isocratic 30% B; Wave Length: 248/205 nm; RT1 (min): 10.6; RT2 (min): 14) to give (Compound 30a) (S)-4-(6-(1-((4-isopropylphenyl)carbamoyl)-2-methylazetidin-2-yl)pyridin-3-yl)benzoic acid (1^{st} peak, 3 mg, 21% yield; Method K, Peak 1, 1.17 min) as a white solid and (Compound 30b) (R)-4-(6-(1-((4-isopropylphenyl)carbamoyl)-2-methylazetidin-2-yl)pyridin-3-yl)benzoic acid (2^{nd} peak, 3.3 mg, 24% yield; Method K, Peak 2, 1.51 min) as a white solid.

| Cmpd # | Characterization Data |
|---|---|
| 30a | LCMS (ESI) [M + H]⁺: 430.2 |
| | ¹H NMR (400 MHz, DMSO-d₆) δ 9.02 (s, 1H), 8.22 (dd, J = 8.2, 2.4 Hz, 1H), 8.05 (s, 2H), 7.89 (d, |
| | J = 8.3 Hz, 2H), 7.65 (d, J = 8.2 Hz, 1H), 7.45 (d, J = 8.5 Hz, 2H), 7.12 (d, J = 8.5 Hz, 2H), 4.08 (q, |
| | J = 8.0 Hz, 1H), 3.89-3.81 (m, 1H), 2.81 (hept, J = 6.9 Hz, 1H), 2.59 (d, J = 9.3 Hz, 1H), 2.36- |
| | 2.23 (m, 1H), 1.82 (s, 3H), 1.17 (d, J = 6.9 Hz, 6H). |
| 30b | LCMS (ESI) [M + H]⁺: 430.2 |
| | ¹H NMR (400 MHz, DMSO-d₆) δ 9.02 (s, 1H), 8.21 (d, J = 9.5 Hz, 1H), 8.06 (d, J = 8.1 Hz, 2H), |
| | 7.88 (d, J = 7.9 Hz, 2H), 7.65 (d, J = 8.2 Hz, 1H), 7.45 (d, J = 8.4 Hz, 2H), 7.12 (d, J = 8.4 Hz, 2H), |
| | 4.13-4.01 (m, 1H), 3.95-3.79 (m, 1H), 2.87-2.75 (m, 1H), 2.68-2.56 (m, 1H), 2.36-2.23 (m, |
| | 1H), 1.82 (s, 3H), 1.17 (d, J = 6.8 Hz, 6H). |

### Example 31: (S)-4-(6-(1-((4-isopropylphenyl)carbamoyl)piperidin-2-yl)pyridin-3-yl)benzoic acid & (R)-4-(6-(1-((4-isopropylphenyl)carbamoyl)piperidin-2-yl)pyridin-3-yl)benzoic acid

### Step 1: methyl 4-(6-chloropyridin-3-yl)benzoate

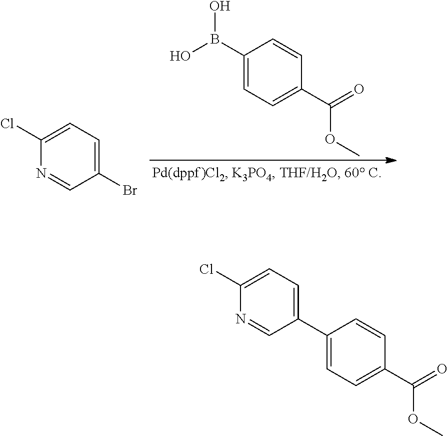 [SMILES: Clc1ccc(Br)cn1 ; COC(=O)c1ccc(B(O)O)cc1 ; COC(=O)c1ccc(-c2ccc(Cl)nc2)cc1]

A solution of 4-(methoxycarbonyl)phenylboronic acid (10.0 g, 55.6 mmol) and 5-bromo-2-chloropyridine (10.69 g, 55.6 mmol) in THF (40 mL) and H₂O (10 mL) was treated with Pd(dppf)Cl₂ (4.07 g, 5.6 mmol) and K₃PO₄ (35.38 g, 166.7 mmol) at room temperature under nitrogen atmosphere. The reaction was heated at 60° C. for 2 hours. Upon completion, the resulting mixture was concentrated under reduced pressure. The crude residue was directly purified by silica gel column chromatography, eluted with PE/EA (2:1) to afford methyl 4-(6-chloropyridin-3-yl)benzoate (6.0 g, 44% yield) as a white solid. LCMS (ESI) [M+H]⁺: 248.6

### Step 2: tert-butyl 2-(5-(4-(methoxycarbonyl)phenyl)pyridin-2-yl)piperidine-1-carboxylate

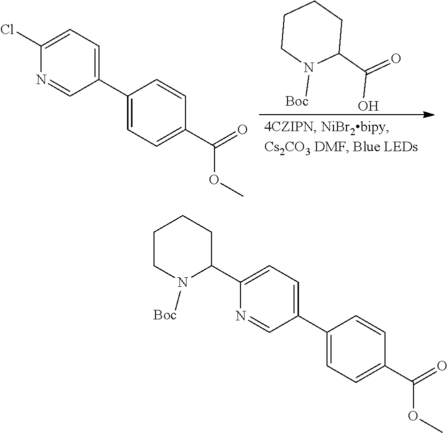 [SMILES: COC(=O)c1ccc(-c2ccc(Cl)nc2)cc1 ; CC(C)(C)OC(=O)N1CCCCC1C(=O)O ; COC(=O)c1ccc(-c2ccc(C3CCCCN3C(=O)OC(C)(C)C)nc2)cc1]

A solution of methyl 4-(6-chloropyridin-3-yl)benzoate (1.0 g, 4.04 mmol), Cs₂CO₃ (1.97 g, 6.06 mmol), NiBr₂·bipy (0.15 g, 0.404 mmol) and tetrakis(9H-carbazol-9-yl)benzene-1,4-dicarbonitrile (0.08 g, 0.101 mmol) in DMF (10 mL) was treated with 1-(tert-butoxycarbonyl)piperidine-2-carboxylic acid (1.39 g, 6.06 mmol) at room temperature under nitrogen atmosphere. The resulting mixture was stirred and irradiated with 450 nm blue LEDs for 24 hours. Upon completion, the precipitated solids were collected by filtration and the filter cake was washed with DMF (2×2 mL). The filtrate was concentrated under reduced pressure. The crude residue was purified by reversed-phase flash chromatography (conditions: column: C18 silica gel; mobile phase A: Water (0.1% FA), Mobile Phase B: MeCN; Gradient (B %): 40% to 60% in 20 min; detector: UV 254 nm) to afford tert-butyl 2-(5-(4-(methoxycarbonyl)phenyl)pyridin-2-yl)piperidine-1-carboxylate (140 mg, 8.8% yield) as a greenish solid. LCMS (ESI) [M+H]⁺: 397.4

### Step 3: methyl 4-(6-(piperidin-2-yl)pyridin-3-yl)benzoate

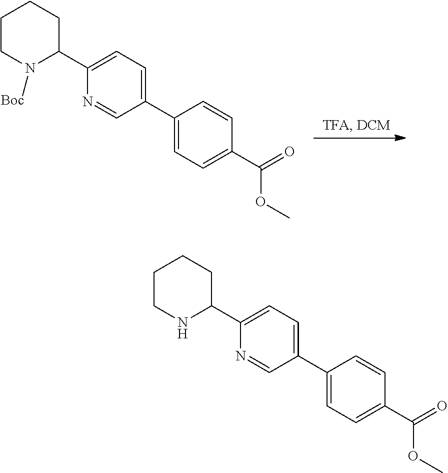 [SMILES: COC(=O)c1ccc(-c2ccc(C3CCCCN3C(=O)OC(C)(C)C)nc2)cc1 ; COC(=O)c1ccc(-c2ccc(C3CCCCN3)nc2)cc1]

A solution of tert-butyl 2-(5-(4-(methoxycarbonyl)phenyl)pyridin-2-yl)piperidine-1-carboxylate (130 mg, 0.33 mmol) in DCM (2 mL) was treated with TFA (2 mL) at room temperature under nitrogen atmosphere. The resulting mixture was stirred for 1 hour. Upon completion, the mixture was concentrated under reduced pressure. The crude residue was directly purified by reversed-phase flash chromatography (conditions: column, C18 silica gel; mobile phase A: Water (0.1% FA), Mobile Phase B: MeCN; Gradient (B %): 18% to 28% in 10 min; detector: UV 254 nm) to afford methyl 4-(6-(piperidin-2-yl)pyridin-3-yl)benzoate (80 mg, 82% yield) as a white solid. LCMS (ESI) [M+H]⁺: 297.3

### Step 4: methyl 4-(6-(1-((4-isopropylphenyl)carbamoyl)piperidin-2-yl)pyridin-3-yl)benzoate

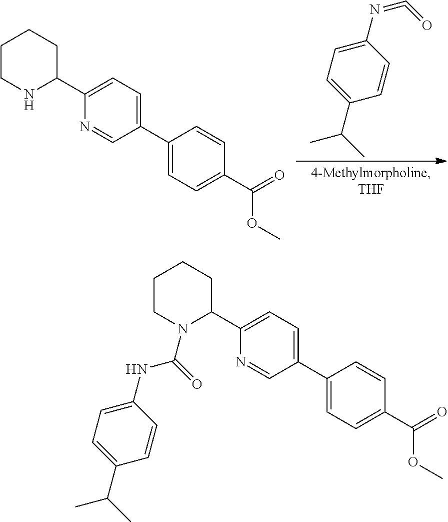

A solution of 1-isocyanato-4-isopropylbenzene (57.1 mg, 0.35 mmol) and methyl 4-(6-(piperidin-2-yl)pyridin-3-yl)benzoate (70 mg, 0.24 mmol) in THF (4 mL) was treated with NMM (71.7 mg, 0.71 mmol) at room temperature under nitrogen atmosphere. The resulting mixture was stirred for 1 hour. Upon completion, the resulting mixture was concentrated under reduced pressure. The crude residue was directly purified by reversed-phase flash chromatography (conditions: column, C18 silica gel; mobile phase A: Water (0.1% FA), Mobile Phase B: MeCN; Gradient (B %): 30% to 60% in 20 min; detector: UV 254 nm) to afford methyl 4-(6-(1-((4-isopropylphenyl)carbamoyl)piperidin-2-yl)pyridin-3-yl)benzoate (70 mg, 65% yield) as a white solid. LCMS (ESI) [M+H]⁺: 458.5

### Step 5: 4-(6-(1-((4-isopropylphenyl)carbamoyl)piperidin-2-yl)pyridin-3-yl)benzoic acid

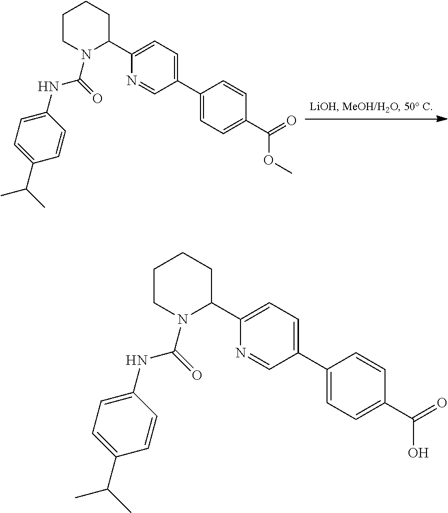

A solution of methyl 4-(6-(1-((4-isopropylphenyl)carbamoyl)piperidin-2-yl)pyridin-3-yl)benzoate (70 mg, 0.153 mmol) and LiOH monohydrate (32.0 mg, 0.765 mmol) in MeOH (4 mL) and H₂O (2 mL) at room temperature under nitrogen atmosphere. The reaction was stirred at 50° C. for 1 hour. Upon completion, the resulting mixture was concentrated under reduced pressure. The crude residue was directly purified by reversed-phase flash chromatography (conditions: column: C18 silica gel; mobile phase A: Water (0.1% FA), Mobile Phase B: MeCN; Gradient (B %): 30% to 50% in 20 min; detector: UV 254 nm) to afford 4-(6-(1-((4-isopropylphenyl)carbamoyl)piperidin-2-yl)pyridin-3-yl)benzoic acid (60 mg, 88% yield) as a white solid. LCMS (ESI) [M+H]⁺: 444.5

### Step 6: (S)-4-(6-(1-((4-isopropylphenyl)carbamoyl)piperidin-2-yl)pyridin-3-yl)benzoic acid & (R)-4-(6-(1-((4-isopropylphenyl)carbamoyl)piperidin-2-yl)pyridin-3-yl)benzoic acid

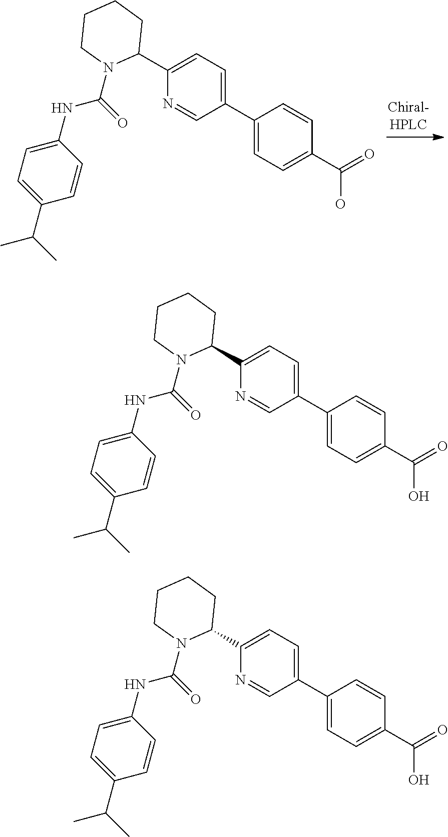 [SMILES: CC(C)c1ccc(NC(=O)N2CCCCC2c2ccc(-c3ccc(C(=O)O)cc3)cn2)cc1 ; CC(C)c1ccc(NC(=O)N2CCCC[C@H]2c2ccc(-c3ccc(C(=O)O)cc3)cn2)cc1 ; CC(C)c1ccc(NC(=O)N2CCCC[C@@H]2c2ccc(-c3ccc(C(=O)O)cc3)cn2)cc1]

4-(6-(1-((4-isopropylphenyl)carbamoyl)piperidin-2-yl)pyridin-3-yl)benzoic acid (60 mg, 88% yield) was separated by prep Chiral-HPLC (conditions: Column: CHIRAL ART Cellulose-SB, 3*25 cm, 5 μm; Mobile Phase A: Hexane (0.1% FA), Mobile Phase B: EtOH; Flow rate: 40 mL/min; Gradient (B %): isocratic 20% B; Wave Length: 240/250 nm; RT1 (min): 10; RT2 (min): 14.8) to give (Compound 31a) (S)-4-(6-(1-((4-isopropylphenyl)carbamoyl)piperidin-2-yl)pyridin-3-yl)benzoic acid (1^{st} peak, 23.3 mg, 39% yield; Method L, Peak 1, 1.19 min) as a white solid and (Compound 31b) (R)-4-(6-(1-((4-isopropylphenyl)carbamoyl)piperidin-2-yl)pyridin-3-yl)benzoic acid (2^{nd} peak, 22.0 mg, 37% yield; Method L, Peak 2, 1.71 min) as a white solid.

| Cmpd # | Characterization Data |
|---|---|
| 31a | LCMS (ESI) [M + H]⁺: 444.2 |
| | ¹H NMR (400 MHz, DMSO-d₆) δ 8.96 (d, J = 2.0 Hz, 1H), 8.55 (s, 1H), 8.16 (dd, J = 8.2, 2.3 Hz, |
| | 1H), 8.04 (d, J = 8.4 Hz, 2H), 7.86 (d, J = 8.3 Hz, 2H), 7.41-7.32 (m, 3H), 7.09 (d, J = 8.5 Hz, |
| | 2H), 5.58 (d, J = 3.8 Hz, 1H), 4.12 (d, J = 13.1 Hz, 1H), 2.96 (t, J = 12.6 Hz, 1H), 2.81 (hept, J = |
| | 6.9 Hz, 1H), 2.66 (d, J = 12.4 Hz, 1H), 1.88-1.74 (m, 1H), 1.67-1.56 (m, 2H), 1.50 (d, J = 13.4 |
| | Hz, 1H), 1.31-1.24 (m, 1H), 1.17 (d, J = 6.9 Hz, 6H). |
| 31b | LCMS (ESI) [M + H]⁺: 444.3 |
| | ¹H NMR (400 MHz, DMSO-d₆) δ 8.96 (d, J = 2.2 Hz, 1H), 8.55 (s, 1H), 8.16 (dd, J = 8.3, 2.4 Hz, |
| | 1H), 8.04 (d, J = 8.3 Hz, 2H), 7.86 (d, J = 8.4 Hz, 2H), 7.40-7.32 (m, 3H), 7.09 (d, J = 8.5 Hz, |
| | 2H), 5.60-5.54 (m, 1H), 4.12 (d, J = 13.3 Hz, 1H), 2.97 (t, J = 12.7 Hz, 1H), 2.81 (hept, J = 6.9 |
| | Hz, 1H), 2.66 (d, J = 12.7 Hz, 1H), 1.83 (d, J = 15.3 Hz, 1H), 1.65-1.57 (m, 2H), 1.50 (d, J = 12.3 |
| | Hz, 1H), 1.32-1.22 (m, 1H), 1.17 (d, J = 6.9 Hz, 6H). |

### Example 32: (S)-4-(6-(1-((3-fluoro-4-isopropylphenyl)carbamoyl)pyrrolidin-2-yl)pyridin-3-yl)benzoic acid & (R)-4-(6-(1-((3-fluoro-4-isopropylphenyl)carbamoyl)pyrrolidin-2-yl)pyridin-3-yl)benzoic acid

### Step 1: 3-fluoro-4-(prop-1-en-2-yl)aniline

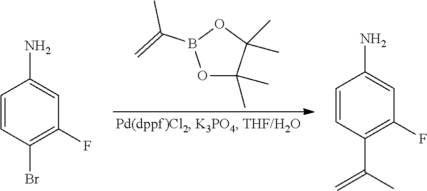

4-Bromo-3-fluoroaniline (2.0 g, 10.53 mmol), 4,4,5,5-tetramethyl-2-(prop-1-en-2-yl)-1,3,2-dioxaborolane (3.54 g, 21.05 mmol), Pd(dppf)Cl₂ (0.86 g, 1.052 mmol) and potassium phosphate tribasic (6.70 g, 31.58 mmol) were dissolved in tetrahydrofuran (8 mL) and water (0.8 mL) at room temperature under a nitrogen atmosphere. The resulting solution was stirred at 80° C. for 1 hour. Upon completion, the mixture was concentrated under reduced pressure. The crude residue was directly purified by silica gel chromatography, eluted with (dichloromethane:methanol=15:1) to give 3-fluoro-4-(prop-1-en-2-yl)aniline (1.3 g, 82% yield) as a grey oil. LCMS (ESI) [M+H]⁺: 152.2

### Step 2: 3-fluoro-4-isopropylaniline

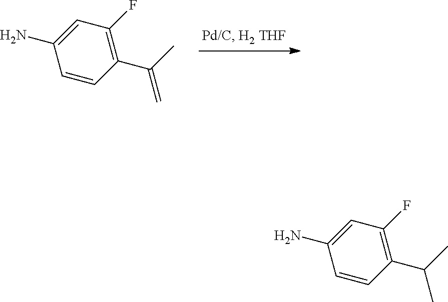

To a solution of 3-fluoro-4-(prop-1-en-2-yl)aniline (1.3 g, 8.60 mmol) in tetrahydrofuran (10 mL) was added Pd/C (10 wt %, 1.83 g, 17.2 mmol) under nitrogen atmosphere. The reaction system was degassed under vacuum and purged with H₂ gas. The mixture was hydrogenated under H₂ balloon at 25° C. for 3 hours. Upon completion, the reaction filtered over celite. The filter cake was washed with methanol (20 mL). The corresponding filtrate was concentrated under reduced pressure and the crude residue was purified by silica gel chromatography, eluted with (petroleum ether/ethyl acetate=35:1) to afford 3-fluoro-4-isopropylaniline (1.1 g, 84% yield) as a grey oil. LCMS (ESI) [M+H]⁺: 154.1

### Step 3: methyl 4-(6-(1-((3-fluoro-4-isopropylphenyl)carbamoyl)pyrrolidin-2-yl)pyridin-3-yl)benzoate

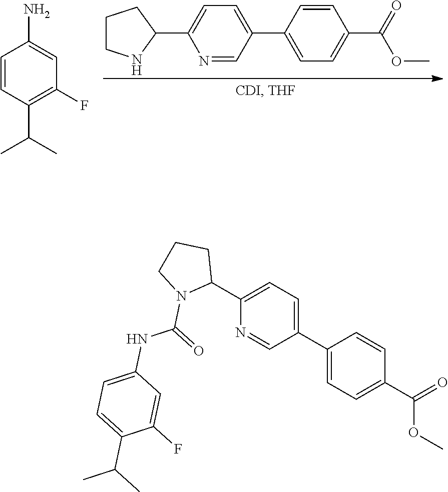

A mixture of 3-fluoro-4-isopropylaniline (369 mg, 2.41 mmol), CDI (488 mg, 3.01 mmol) in tetrahydrofuran (3 mL) was stirred at room temperature for 10 minutes. Then, methyl 4-[6-(pyrrolidin-2-yl)pyridin-3-yl]benzoate (170 mg, 0.602 mmol) was added. The resulting mixture was stirred for an additional 1 hour. Upon completion, the mixture was poured into water and extracted with DCM (3×20 mL). The combined organic layers were washed with brine, dried over anhydrous Na₂SO₄, filtered and concentrated under reduced pressure. The crude residue was purified by reversed-phase chromatography (conditions: column: C18 silica gel; Mobile Phase A: Water (0.1% FA), Mobile Phase B: acetonitrile; Gradient (B %): 25% to 100% in 30 min; Detector: 254 nm) to afford methyl 4-(6-(1-((3-fluoro-4-isopropylphenyl)carbamoyl)pyrrolidin-2-yl)pyridin-3-yl)benzoate (120 mg, 43% yield) as a light yellow solid. LCMS (ESI) [M+H]⁺: 462.2

### Step 4: 4-(6-(1-((3-fluoro-4-isopropylphenyl)carbamoyl)pyrrolidin-2-yl)pyridin-3-yl)benzoic acid

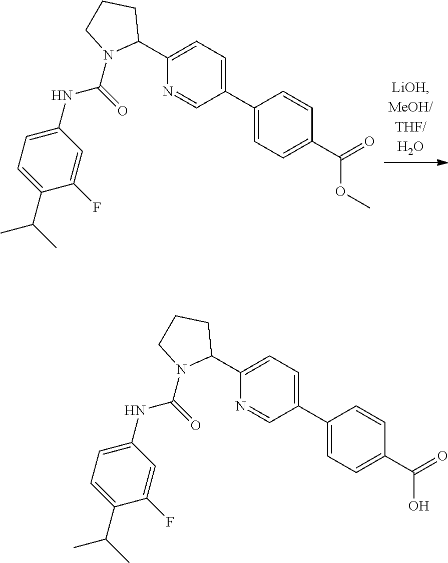

A mixture of methyl 4-(6-(1-((3-fluoro-4-isopropylphenyl)carbamoyl)pyrrolidin-2-yl)pyridin-3-yl)benzoate (100 mg, 0.217 mmol), LiOH monohydrate (46 mg, 1.09 mmol) in tetrahydrofuran (1 mL), methanol (1 mL) and water (1 mL) was stirred at room temperature for 1 hour. Upon completion, the mixture was directly purified by reversed-phase chromatography (conditions: column: C18 silica gel; Mobile Phase A: Water (0.1% FA), Mobile Phase B: acetonitrile; Gradient (B %): 25% to 100% in 30 min, Detector: 254 nm) to afford 4-(6-(1-((3-fluoro-4-isopropylphenyl)carbamoyl)pyrrolidin-2-yl)pyridin-3-yl)benzoic acid (60 mg, 62% yield) as a light grey solid. LCMS (ESI) [M+H]⁺: 448.2

### Step 5: (S)-4-(6-(1-((3-fluoro-4-isopropylphenyl)carbamoyl)pyrrolidin-2-yl)pyridin-3-yl)benzoic acid & (R)-4-(6-(1-((3-fluoro-4-isopropylphenyl)carbamoyl)pyrrolidin-2-yl)pyridin-3-yl)benzoic acid

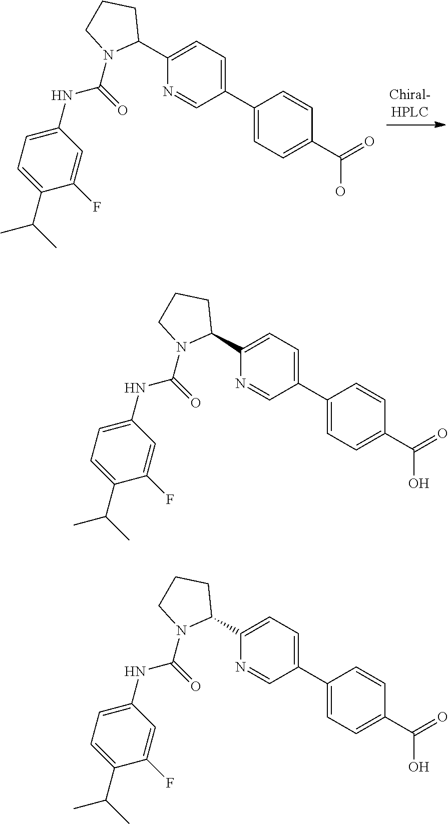 [SMILES: CC(C)c1ccc(NC(=O)N2CCCC2c2ccc(-c3ccc(C(=O)O)cc3)cn2)cc1F ; CC(C)c1ccc(NC(=O)N2CCC[C@H]2c2ccc(-c3ccc(C(=O)O)cc3)cn2)cc1F ; CC(C)c1ccc(NC(=O)N2CCC[C@@H]2c2ccc(-c3ccc(C(=O)O)cc3)cn2)cc1F]

4-(6-(1-((3-fluoro-4-isopropylphenyl)carbamoyl)pyrrolidin-2-yl)pyridin-3-yl)benzoic acid (60 mg) was separated by prep Chiral-HPLC (conditions: Column: CHIRAL ART Cellulose-SB, 3*25 cm, 5 μm; Mobile Phase A: MTBE (0.1% FA), Mobile Phase B: MeOH; Flow rate: 40 mL/min; Gradient (B %): isocratic 10% B; Wave Length: 246/204 nm; RT1 (min): 5.73; RT2 (min): 7.86) to afford (Compound 32a) (S)-4-(6-(1-((3-fluoro-4-isopropylphenyl)carbamoyl)pyrrolidin-2-yl)pyridin-3-yl)benzoic acid (1^{st} peak, 10.5 mg, 13% yield; Method J, Peak 1, 0.58 min) as a white solid and (Compound 32b) (R)-4-(6-(1-((3-fluoro-4-isopropylphenyl)carbamoyl)pyrrolidin-2-yl)pyridin-3-yl)benzoic acid (2^{nd} peak, 11.2 mg, 14% yield; Method J, Peak 2, 0.82 min) as a white solid.

| Cmpd # | Characterization Data |
|---|---|
| 32a | LCMS (ESI) [M + H]⁺: 448.1 |
| | ¹H NMR (400 MHz, DMSO-d₆) δ 13.00 (bs, 1H), 8.88 (d, J = 2.0 Hz, 1H), 8.42 (s, 1H), 8.09 (dd, |
| | J = 8.2, 2.3 Hz, 1H), 8.03 (d, J = 8.3 Hz, 2H), 7.84 (d, J = 8.3 Hz, 2H), 7.42-7.31 (m, 2H), 7.20 (dd, |
| | J = 8.5, 1.8 Hz, 1H), 7.13 (t, J = 8.6 Hz, 1H), 5.20-5.08 (m, 1H), 3.85-3.69 (m, 1H), 3.66-3.52 |
| | (m, 1H), 3.05 (hept, J = 6.9 Hz, 1H), 2.38-2.22 (m, 1H), 2.02-1.89 (m, 3H), 1.16 (d, J = 6.9 Hz, |
| | 6H). |
| 32b | LCMS (ESI) [M + H]⁺: 448.1 |
| | ¹H NMR (400 MHz, DMSO-d₆) δ 12.98 (bs, 1H), 8.88 (d, J = 2.1 Hz, 1H), 8.42 (s, 1H), 8.09 (dd, J = 8.2, |
| | 2.3 Hz, 1H), 8.03 (d, J = 8.3 Hz, 2H), 7.84 (d, J = 8.3 Hz, 2H), 7.42-7.32 (m, 2H), 7.20 (dd, |
| | J = 8.5, 1.8 Hz, 1H), 7.13 (t, J = 8.6 Hz, 1H), 5.14 (d, J = 9.0 Hz, 1H), 3.78 (q, J = 7.1, 5.2 Hz, 1H), |
| | 3.59 (q, J = 7.9 Hz, 1H), 3.05 (hept, J = 6.9 Hz, 1H), 2.37-2.22 (m, 1H), 2.05-1.80 (m, 3H), 1.16 |
| | (d, J = 6.9 Hz, 6H). |

### Example 37: (S)-4-(6-(1-((4-chloro-3-(trifluoromethyl)phenyl)carbamoyl)pyrrolidin-2-yl)-5-fluoropyridin-3-yl)benzoic acid & (R)-4-(6-(1-((4-chloro-3-(trifluoromethyl)phenyl)carbamoyl)pyrrolidin-2-yl)-5-fluoropyridin-3-yl)benzoic acid

### Step 1: methyl 4-(6-(1-((4-chloro-3-(trifluoromethyl)phenyl)carbamoyl)pyrrolidin-2-yl)-5-fluoropyridin-3-yl)benzoate

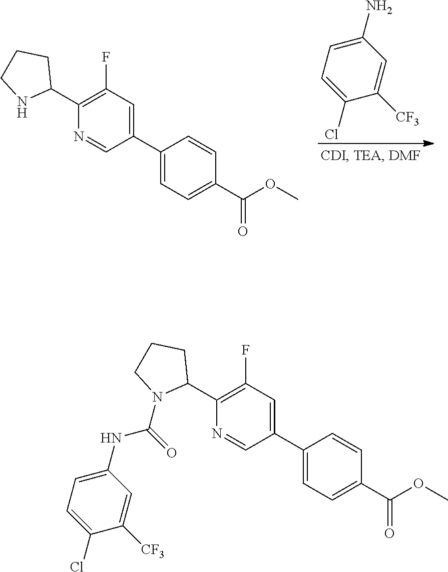 [SMILES: COC(=O)c1ccc(-c2cnc(C3CCCN3)c(F)c2)cc1 ; Nc1ccc(Cl)c(C(F)(F)F)c1 ; COC(=O)c1ccc(-c2cnc(C3CCCN3C(=O)Nc3ccc(Cl)c(C(F)(F)F)c3)c(F)c2)cc1]

A mixture of methyl 4-[5-fluoro-6-(pyrrolidin-2-yl)pyridin-3-yl]benzoate (200 mg, 0.67 mmol), CDI (540 mg, 3.33 mmol) and triethylamine (337 mg, 3.33 mmol) in DMF (5 mL) were stirred at 60° C. After 30 minutes, 4-chloro-3-(trifluoromethyl)aniline (391 mg, 2.00 mmol) was added and the resulting mixture was stirred at 60° C. for an additional 1 hour. Upon completion, the mixture was allowed to cool down to room temperature and was concentrated under reduced pressure. The crude residue was purified by reversed-phase flash chromatography (conditions: column: C18 silica gel; mobile phase A: Water (0.1% FA), Mobile Phase B: MeCN; Gradient (B %): 20% to 70% in 10 min; detector: UV 254 nm) to afford methyl 4-(6-(1-((4-chloro-3-(trifluoromethyl)phenyl)carbamoyl)pyrrolidin-2-yl)-5-fluoropyridin-3-yl)benzoate (100 mg, crude) as a yellow oil. LCMS (ESI) [M+H]⁺: 522.1

### Step 2: 4-(6-(1-((4-chloro-3-(trifluoromethyl)phenyl)carbamoyl)pyrrolidin-2-yl)-5-fluoropyridin-3-yl)benzoic acid

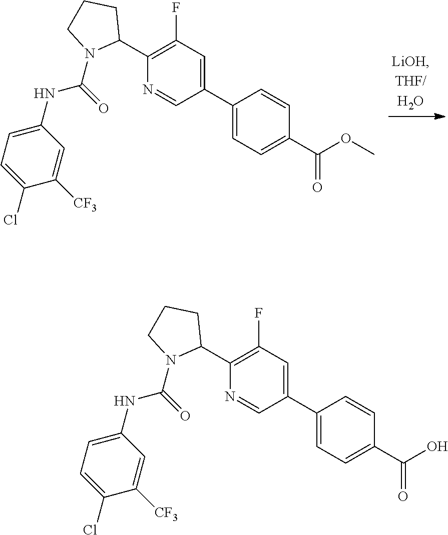 [SMILES: COC(=O)c1ccc(-c2cnc(C3CCCN3C(=O)Nc3ccc(Cl)c(C(F)(F)F)c3)c(F)c2)cc1 ; O=C(O)c1ccc(-c2cnc(C3CCCN3C(=O)Nc3ccc(Cl)c(C(F)(F)F)c3)c(F)c2)cc1]

A solution of methyl 4-(6-(1-((4-chloro-3-(trifluoromethyl)phenyl)carbamoyl)pyrrolidin-2-yl)-5-fluoropyridin-3-yl)benzoate (100 mg, 0.192 mmol) and LiOH monohydrate (40.3 mg, 0.96 mmol) in THF (1 mL) and H₂O (0.2 mL) was stirred at 50° C. for 2 hours. Upon completion, the mixture was allowed to cool down to room temperature and the reaction was quenched with water. The resulting mixture was extracted with EtOAc (3×5 mL). The combined organic layers were washed with water (3×5 mL), dried over anhydrous Na₂SO₄, filtered and concentrated under reduced pressure. The crude residue was purified by reversed-phase flash chromatography (conditions: column: C18 silica gel; mobile phase A: Water (0.1% FA), Mobile Phase B: MeCN; Gradient (B %): 10% to 60% in 20 min; detector: UV 254 nm) to afford 4-(6-(1-((4-chloro-3-(trifluoromethyl)phenyl)carbamoyl)pyrrolidin-2-yl)-5-fluoropyridin-3-yl)benzoic acid (80 mg, 82% yield) as a yellow solid. LCMS (ESI) [M+H]⁺: 508.1

### Step 3: (S)-4-(6-(1-((4-chloro-3-(trifluoromethyl)phenyl)carbamoyl)pyrrolidin-2-yl)-5-fluoropyridin-3-yl)benzoic acid & (R)-4-(6-(1-((4-chloro-3-(trifluoromethyl)phenyl)carbamoyl)pyrrolidin-2-yl)-5-fluoropyridin-3-yl)benzoic acid

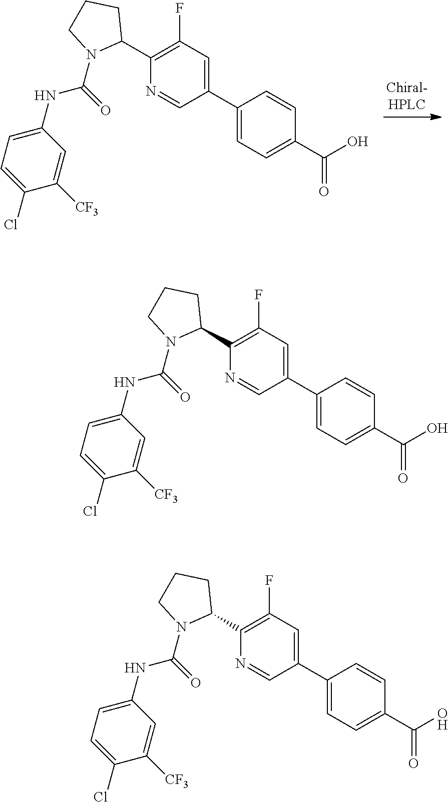 [SMILES: O=C(O)c1ccc(-c2cnc(C3CCCN3C(=O)Nc3ccc(Cl)c(C(F)(F)F)c3)c(F)c2)cc1 ; O=C(O)c1ccc(-c2cnc([C@@H]3CCCN3C(=O)Nc3ccc(Cl)c(C(F)(F)F)c3)c(F)c2)cc1 ; O=C(O)c1ccc(-c2cnc([C@H]3CCCN3C(=O)Nc3ccc(Cl)c(C(F)(F)F)c3)c(F)c2)cc1]

4-(6-(1-((4-chloro-3-(trifluoromethyl)phenyl)carbamoyl)pyrrolidin-2-yl)-5-fluoropyridin-3-yl)benzoic acid (80 mg, 0.158 mmol) was separated by prep Chiral-HPLC (conditions: Column: CHIRALPAK IK 3*25 cm, 5 μm; Mobile Phase A: Hexane (0.1% FA), Mobile Phase B: EtOH; Flow rate: 40 mL/min; Gradient (B %): isocratic 10% B; Wave Length: 253/286 nm; RT1 (min): 17.11; RT2 (min): 26.84) to afford (Compound 37a) (S)-4-(6-(1-((4-chloro-3-(trifluoromethyl)phenyl)carbamoyl)pyrrolidin-2-yl)-5-fluoropyridin-3-yl)benzoic acid (1^{st} peak, 29.4 mg, 37% yield; Method 0, Peak 1, 1.84 min) as a white solid and (Compound 37b) (R)-4-(6-(1-((4-chloro-3-(trifluoromethyl)phenyl)carbamoyl)pyrrolidin-2-yl)-5-fluoropyridin-3-yl)benzoic acid (2^{nd} peak, 27.8 mg, 35% yield; Method 0, Peak 2, 2.85 min) as a white solid.

| Cmpd # | Characterization Data |
|---|---|
| 37a | LCMS (ESI) [M + H]⁺: 508.1 |
| | ¹H NMR (400 MHz, DMSO-d₆) δ 13.09 (bs, 1H), 8.74 (dd, J = 3.4, 1.8 Hz, 2H), 8.08 (dd, J = 11.7, |
| | 2.1 Hz, 2H), 8.05-7.99 (m, 2H), 7.92-7.86 (m, 2H), 7.78 (dd, J = 8.8, 2.6 Hz, 1H), 7.52 (d, J = |
| | 8.9 Hz, 1H), 5.36 (dd, J = 8.0, 4.1 Hz, 1H), 3.87-3.73 (m, 1H), 3.71-3.60 (m, 1H), 2.43-2.29 |
| | (m, 1H), 2.21-1.97 (m, 2H), 1.97-1.83 (m, 1H). |
| 37b | LCMS (ESI) [M + H]⁺: 508.1 |
| | ¹H NMR (400 MHz, DMSO-d₆) δ 13.06 (bs, 1H), 8.78-8.70 (m, 2H), 8.12-8.06 (m, 2H), 8.05- |
| | 8.00 (m, 2H), 7.95-7.86 (m, 2H), 7.78 (dd, J = 8.9, 2.7 Hz, 1H), 7.52 (d, J = 8.8 Hz, 1H), 5.41- |
| | 5.27 (m, 1H), 3.83-3.73 (m, 1H), 3.71-3.59 (m, 1H), 2.43-2.27 (m, 1H), 2.21-1.98 (m, 2H), |
| | 1.96-1.79 (m, 1H). |

### Example 38: (S)-4-(5-fluoro-6-(1-((3-fluoro-4-(trifluoromethyl)phenyl)carbamoyl)pyrrolidin-2-yl)pyridin-3-yl)benzoic acid & (R)-4-(5-fluoro-6-(1-((3-fluoro-4-(trifluoromethyl)phenyl)carbamoyl)pyrrolidin-2-yl)pyridin-3-yl)benzoic acid

### Step 1: methyl 4-(5-fluoro-6-(1-((3-fluoro-4-(trifluoromethyl)phenyl)carbamoyl)pyrrolidin-2-yl)pyridin-3-yl)benzoate

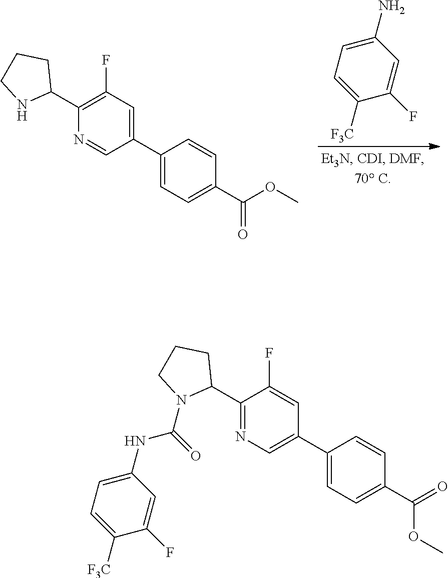 [SMILES: COC(=O)c1ccc(-c2cnc(C3CCCN3)c(F)c2)cc1 ; Nc1ccc(C(F)(F)F)c(F)c1 ; COC(=O)c1ccc(-c2cnc(C3CCCN3C(=O)Nc3ccc(C(F)(F)F)c(F)c3)c(F)c2)cc1]

A solution of 3-fluoro-4-(trifluoromethyl)aniline (358 mg, 2.00 mmol), CDI (324 mg, 2.00 mmol) in DMF (2 mL) was treated with Et₃N (202 mg, 2.00 mmol) followed by the dropwise addition of methyl 4-[5-fluoro-6-(pyrrolidin-2-yl)pyridin-3-yl]benzoate (120 mg, 0.400 mmol). The resulting mixture was stirred at 70° C. for 2 hours. Upon completion, the mixture was cooled to room temperature and was directly purified by reversed-phase flash chromatography (conditions: column: C18 silica gel; mobile phase A: Water (0.1% FA), Mobile Phase B: MeCN; Gradient (B %): 25% to 100% in 25 min; detector: UV 254 nm) to afford methyl 4-(5-fluoro-6-(1-((3-fluoro-4-(trifluoromethyl)phenyl)carbamoyl)pyrrolidin-2-yl)pyridin-3-yl)benzoate (100 mg, 50% yield) as a light yellow oil. LCMS (ESI) [M+H]⁺: 506.1

### Step 2: 4-(5-fluoro-6-(1-((3-fluoro-4-(trifluoromethyl)phenyl)carbamoyl)pyrrolidin-2-yl)pyridin-3-yl)benzoic acid

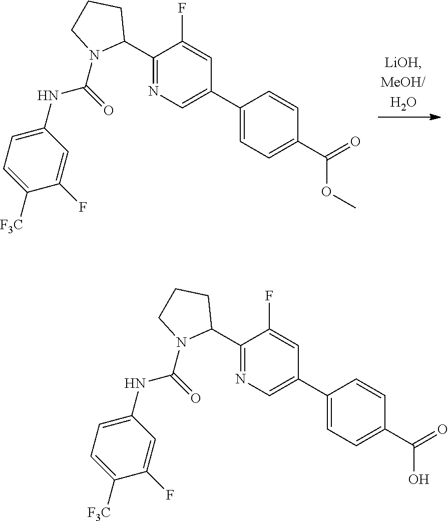 [SMILES: COC(=O)c1ccc(-c2cnc(C3CCCN3C(=O)Nc3ccc(C(F)(F)F)c(F)c3)c(F)c2)cc1 ; O=C(O)c1ccc(-c2cnc(C3CCCN3C(=O)Nc3ccc(C(F)(F)F)c(F)c3)c(F)c2)cc1]

To a solution of methyl 4-(5-fluoro-6-(1-((3-fluoro-4-(trifluoromethyl)phenyl)carbamoyl)pyrrolidin-2-yl)pyridin-3-yl)benzoate (100 mg, 0.198 mmol) in MeOH (2 mL) and H₂O (2 mL) was added LiOH monohydrate (83.0 mg, 1.98 mmol). The resulting mixture was stirred at room temperature for 2 hours. Upon completion, the mixture was acidified to pH=4 with conc. HCl acid and then concentrated under reduced pressure. The crude residue was directly purified by reversed-phase flash chromatography (conditions: column: C18 silica gel; mobile phase A: Water (0.1% FA), Mobile Phase B: MeCN; Gradient (B %): 15% to 50% in 25 min; detector: UV 254 nm) to afford 4-(5-fluoro-6-(1-((3-fluoro-4-(trifluoromethyl)phenyl)carbamoyl)pyrrolidin-2-yl)pyridin-3-yl)benzoic acid (75 mg, 77% yield) as a white solid. LCMS (ESI) [M+H]⁺: 492.1

### Step 3: (S)-4-(5-fluoro-6-(1-((3-fluoro-4-(trifluoromethyl)phenyl)carbamoyl)pyrrolidin-2-yl)pyridin-3-yl)benzoic acid & (R)-4-(5-fluoro-6-(1-((3-fluoro-4-(trifluoromethyl)phenyl)carbamoyl)pyrrolidin-2-yl)pyridin-3-yl)benzoic acid

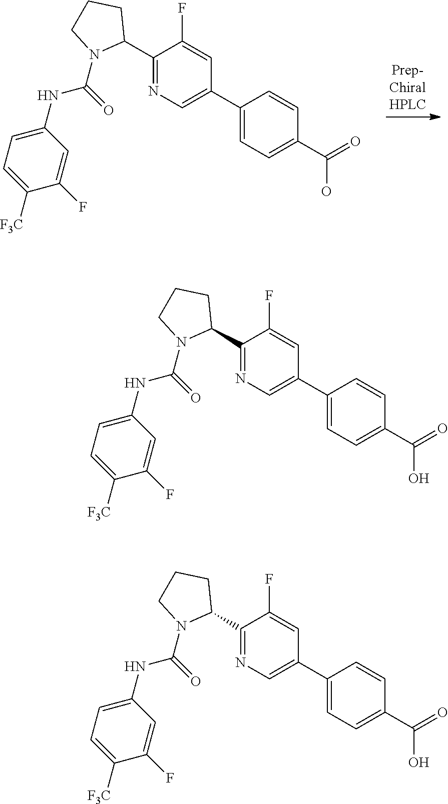 [SMILES: O=C(O)c1ccc(-c2cnc(C3CCCN3C(=O)Nc3ccc(C(F)(F)F)c(F)c3)c(F)c2)cc1 ; O=C(O)c1ccc(-c2cnc([C@@H]3CCCN3C(=O)Nc3ccc(C(F)(F)F)c(F)c3)c(F)c2)cc1 ; O=C(O)c1ccc(-c2cnc([C@H]3CCCN3C(=O)Nc3ccc(C(F)(F)F)c(F)c3)c(F)c2)cc1]

4-(5-fluoro-6-(1-((3-fluoro-4-(trifluoromethyl)phenyl)carbamoyl)pyrrolidin-2-yl)pyridin-3-yl)benzoic acid (75 mg) was separated by Prep-Chiral HPLC (conditions: Column: CHIRALPAK IK 3*25 cm, 5 μm; Mobile Phase A: Hexane (0.1% FA), Mobile Phase B: EtOH; Flow rate: 40 mL/min; Gradient (B %): isocratic 20% B; Wave Length: 250/284 nm; RT1 (min): 6.43; RT2 (min): 8.55) to give (Compound 38a) (S)-4-(5-fluoro-6-(1-((3-fluoro-4-(trifluoromethyl)phenyl)carbamoyl)pyrrolidin-2-yl)pyridin-3-yl)benzoic acid (1^{st} peak, 28.6 mg, 38% yield; Method P, Peak 1, 0.80 min) as a white solid and (Compound 38b) (R)-4-(5-fluoro-6-(1-((3-fluoro-4-(trifluoromethyl)phenyl)carbamoyl)pyrrolidin-2-yl)pyridin-3-yl)benzoic acid (2^{nd} peak, 28.2 mg, 38% yield; Method P, Peak 2, 1.03 min) as a white solid.

| Cmpd # | Characterization Data |
|---|---|
| 38a | LCMS (ESI) [M + H]⁺: 492.1 |
| | ¹H NMR (400 MHz, DMSO-d₆) δ 13.06 (bs, 1H), 8.90 (s, 1H), 8.74 (s, 1H), 8.10 (dd, J = 11.5, 1.8 |
| | Hz, 1H), 8.03 (d, J = 8.5 Hz, 2H), 7.90 (d, J = 8.5 Hz, 2H), 7.68 (d, J = 14.5 Hz, 1H), 7.58 (t, J = 8.7 |
| | Hz, 1H), 7.44 (d, J = 8.7 Hz, 1H), 5.36 (bs, 1H), 3.83-3.73 (m, 1H), 3.71-3.65 (m, 1H), 2.41- |
| | 2.30 (m, 1H), 2.16-1.96 (m, 2H), 1.96-1.84 (m, 1H). |
| 38b | LCMS (ESI) [M + H]⁺: 492.1 |
| | ¹H NMR (400 MHz, DMSO-d₆) δ 13.07 (bs, 1H), 8.90 (s, 1H), 8.74 (s, 1H), 8.10 (dd, J = 11.5, 1.8 |
| | Hz, 1H), 8.03 (d, J = 8.4 Hz, 2H), 7.90 (d, J = 8.4 Hz, 2H), 7.68 (d, J = 14.4 Hz, 1H), 7.58 (t, J = 8.7 |
| | Hz, 1H), 7.44 (d, J = 8.7 Hz, 1H), 5.36 (bs, 1H), 3.85-3.74 (m, 1H), 3.74-3.60 (m, 1H), 2.40- |
| | 2.32 (m, 1H), 2.16-1.98 (m, 2H), 1.96-1.84 (m, 1H). |

### Example 40: (S)-5-(6-(1-((4-isopropylphenyl)carbamoyl)pyrrolidin-2-yl)pyridin-3-yl)-2-methylbenzoic acid & (R)-5-(6-(1-((4-isopropylphenyl)carbamoyl)pyrrolidin-2-yl)pyridin-3-yl)-2-methylbenzoic acid

### Step 1: methyl 5-(6-(1-((4-isopropylphenyl)carbamoyl)pyrrolidin-2-yl)pyridin-3-yl)-2-methylbenzoate

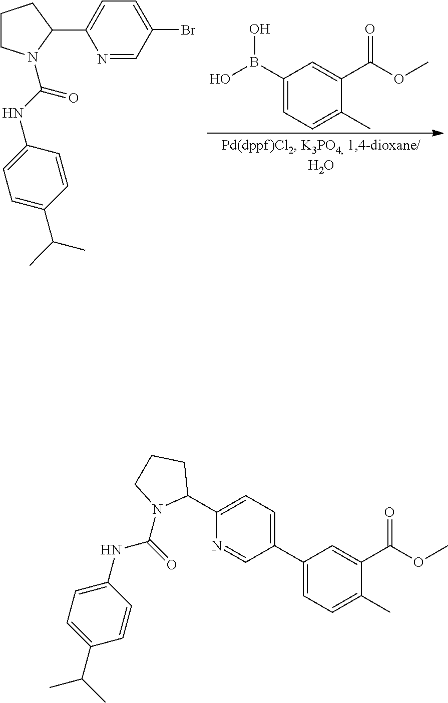 [SMILES: CC(C)c1ccc(NC(=O)N2CCCC2c2ccc(Br)cn2)cc1 ; COC(=O)c1cc(B(O)O)ccc1C ; COC(=O)c1cc(-c2ccc(C3CCCN3C(=O)Nc3ccc(C(C)C)cc3)nc2)ccc1C]

2-(5-bromopyridin-2-yl)-N-(4-isopropylphenyl)pyrrolidine-1-carboxamide (100 mg, 0.26 mmol), (3-(methoxycarbonyl)-4-methylphenyl)boronic acid (125 mg, 0.6 mmol), Pd(dppf)Cl₂ (21.0 mg, 0.026 mmol) and potassium phosphate tribasic (164 mg, 0.77 mmol) were dissolved in 1,4-dioxane (2 mL) and water (0.2 mL). The mixture was stirred at 80° C. for 1 hour under nitrogen atmosphere. Upon completion, the mixture was concentrated under reduced pressure. The crude residue was purified by silica gel chromatography, eluted with (Petroleum ether/Ethyl acetate=15:1) to give methyl 5-(6-(1-((4-isopropylphenyl)carbamoyl)pyrrolidin-2-yl)pyridin-3-yl)-2-methylbenzoate (95 mg, 81% yield) as a yellow solid. LCMS (ESI) [M+H]⁺: 458.6

### Step 2: 5-(6-(1-((4-isopropylphenyl)carbamoyl)pyrrolidin-2-yl)pyridin-3-yl)-2-methylbenzoic acid

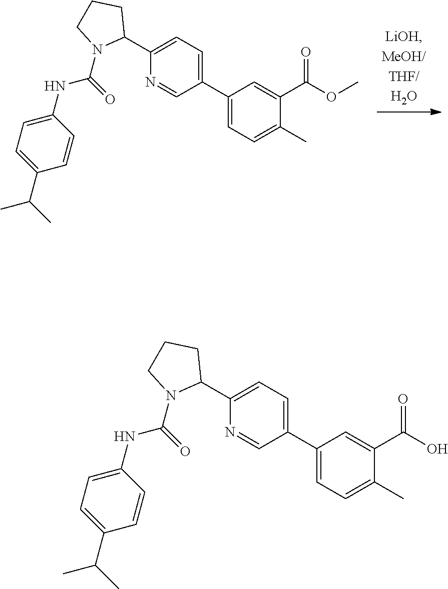

A mixture of methyl 5-(6-(1-((4-isopropylphenyl)carbamoyl)pyrrolidin-2-yl)pyridin-3-yl)-2-methylbenzoate (90 mg, 0.20 mmol), lithium hydroxide monohydrate (42.0 mg, 0.99 mmol) in tetrahydrofuran (1 mL), methanol (1 mL) and water (1 mL) was stirred at room temperature for 1 hour. Upon completion, the mixture was directly purified by reversed-phase column chromatography (conditions: column: C18 Silica gel; Mobile Phase A: Water (0.1% FA), Mobile Phase B: acetonitrile; Gradient (B %): 0% to 100% in 25 min; detector: 254 nm) to afford 5-(6-(1-((4-isopropylphenyl)carbamoyl)pyrrolidin-2-yl)pyridin-3-yl)-2-methylbenzoic acid (80 mg, 87% yield) as an off-white solid. LCMS (ESI) [M+H]⁺: 444.6

### Step 3: (S)-5-(6-(1-((4-isopropylphenyl)carbamoyl)pyrrolidin-2-yl)pyridin-3-yl)-2-methylbenzoic acid & (R)-5-(6-(1-((4-isopropylphenyl)carbamoyl)pyrrolidin-2-yl)pyridin-3-yl)-2-methylbenzoic acid

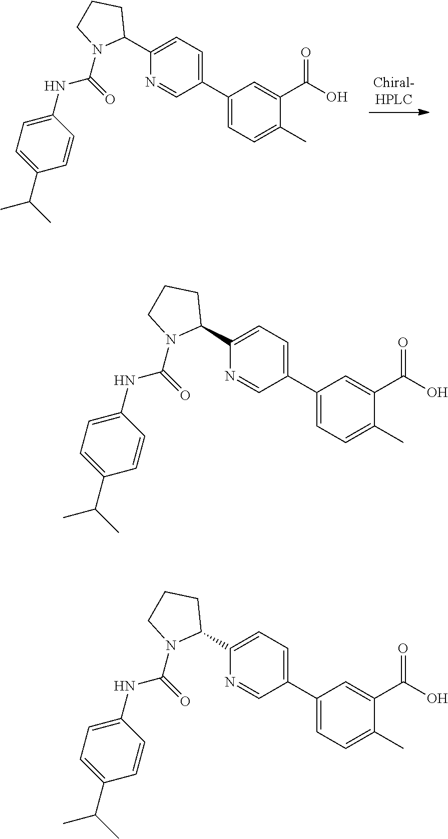 [SMILES: Cc1ccc(-c2ccc(C3CCCN3C(=O)Nc3ccc(C(C)C)cc3)nc2)cc1C(=O)O ; Cc1ccc(-c2ccc([C@@H]3CCCN3C(=O)Nc3ccc(C(C)C)cc3)nc2)cc1C(=O)O ; Cc1ccc(-c2ccc([C@H]3CCCN3C(=O)Nc3ccc(C(C)C)cc3)nc2)cc1C(=O)O]

5-(6-(1-((4-isopropylphenyl)carbamoyl)pyrrolidin-2-yl)pyridin-3-yl)-2-methylbenzoic acid (80 mg, 0.180 mmol) was separated by Prep-CHIRAL HPLC (conditions: Column: CHIRALPAK IK 3*25 cm, 5 μm; Mobile Phase A: Hexane (0.1% FA), Mobile Phase B: EtOH; Flow rate: 40 mL/min; Gradient (B %): isocratic 30% B; Wave Length: 237/254 nm; RT1 (min): 77.2; RT2 (min): 91.2) to afford (Compound 40a) (S)-5-(6-(1-((4-isopropylphenyl)carbamoyl)pyrrolidin-2-yl)pyridin-3-yl)-2-methylbenzoic acid (1^{st} peak, 31.1 mg, 39% yield; Method K, Peak 1, 0.91 min) as a white solid and (Compound 40b) (R)-5-(6-(1-((4-isopropylphenyl)carbamoyl)pyrrolidin-2-yl)pyridin-3-yl)-2-methylbenzoic acid (2^{nd} peak, 30.0 mg, 38% yield; Method K, Peak 2, 1.79 min) as a white solid. LCMS (ESI) [M+H]⁺: 444.6

| Cmpd # | Characterization Data |
|---|---|
| 40a | LCMS (ESI) [M + H]⁺: 444.2 |
| | ¹H NMR (400 MHz, DMSO-d₆) δ 12.99 (bs, 1H), 8.81 (d, J = 2.1 Hz, 1H), 8.22 (s, 1H), 8.07 (d, J = |
| | 1.8 Hz, 1H), 8.03 (dd, J = 8.2, 2.3 Hz, 1H), 7.78 (dd, J = 7.9, 1.9 Hz, 1H), 7.42 (d, J = 8.1 Hz, 1H), |
| | 7.41-7.30 (m, 3H), 7.06 (d, J = 8.5 Hz, 2H), 5.13 (dd, J = 8.3, 2.5 Hz, 1H), 3.83-3.72 (m, 1H), |
| | 3.63-3.52 (m, 1H), 2.79 (hept, J = 6.9 Hz, 1H), 2.55 (s, 3H), 2.39-2.26 (m, 1H), 2.05-1.85 (m, |
| | 3H), 1.15 (d, J = 6.9 Hz, 6H). |
| 40b | LCMS (ESI) [M + H]⁺: 444.2 |
| | ¹H NMR (400 MHz, DMSO-d₆) δ 12.98 (bs, 1H), 8.81 (d, J = 2.1 Hz, 1H), 8.22 (s, 1H), 8.07 (d, J = |
| | 1.9 Hz, 1H), 8.03 (dd, J = 8.2, 2.3 Hz, 1H), 7.78 (dd, J = 7.9, 2.0 Hz, 1H), 7.42 (d, J = 8.1 Hz, 1H), |
| | 7.41-7.31 (m, 3H), 7.06 (d, J = 8.5 Hz, 2H), 5.13 (dd, J = 8.4, 2.6 Hz, 1H), 3.85-3.71 (m, 1H), |
| | 3.63-3.49 (m, 1H), 2.79 (hept, J = 6.9 Hz, 1H), 2.55 (s, 3H), 2.36-2.18 (m, 1H), 2.04-1.80 (m, |
| | 3H), 1.15 (d, J = 6.9 Hz, 6H). |

### Example 44: (S)-4-(5-fluoro-6-(1-((4-(1-(trifluoromethyl)cyclopropyl)phenyl)carbamoyl)pyrrolidin-2-yl)pyridin-3-yl)benzoic acid and (R)-4-(5-fluoro-6-(1-((4-(1-(trifluoromethyl)cyclopropyl)phenyl)carbamoyl)pyrrolidin-2-yl)pyridin-3-yl)benzoic acid

### Step 1: methyl 4-(5-fluoro-6-(1-((4-(1-(trifluoromethyl)cyclopropyl)phenyl)carbamoyl)pyrrolidin-2-yl)pyridin-3-yl)benzoate

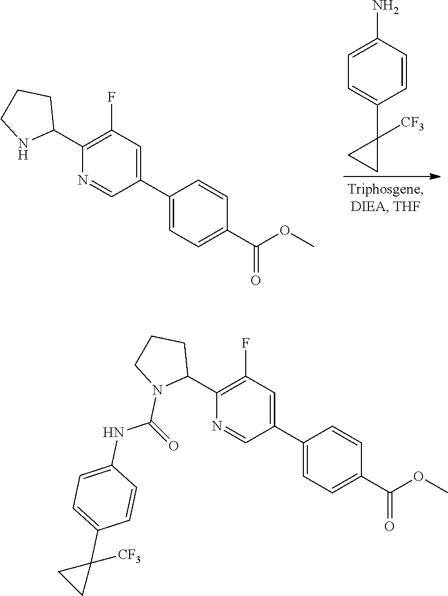 [SMILES: COC(=O)c1ccc(-c2cnc(C3CCCN3)c(F)c2)cc1 ; Nc1ccc(C2(C(F)(F)F)CC2)cc1 ; COC(=O)c1ccc(-c2cnc(C3CCCN3C(=O)Nc3ccc(C4(C(F)(F)F)CC4)cc3)c(F)c2)cc1]

A mixture of ditrichloromethyl carbonate (40 mg, 0.1 mmol) and 4-[1-(trifluoromethyl)cyclopropyl]aniline (67 mg, 0.3 mmol) in THF (2 mL) was stirred at 0° C. for 5 minutes under nitrogen atmosphere. Then, methyl 4-(5-fluoro-6-(pyrrolidin-2-yl)pyridin-3-yl)benzoate (100 mg, 0.3 mmol) was added dropwise. The resulting mixture was warmed to room temperature and was allowed to stir for an additional 1 hour. Upon completion, the reaction was quenched with ice water at 0° C. The resulting mixture was extracted with EtOAc (3×50 mL). The combined organic layers were dried over anhydrous Na₂SO₄, filtered and concentrated under reduced pressure. The crude residue was purified by silica gel column chromatography, eluted with PE/EA (5:1) to afford methyl 4-(5-fluoro-6-(1-((4-(1-(trifluoromethyl)cyclopropyl)phenyl)carbamoyl)pyrrolidin-2-yl)pyridin-3-yl)benzoate (140 mg, 79% yield) as a light yellow solid. LCMS (ESI) [M+H]⁺: 528

### Step 2: 4-(5-fluoro-6-(1-((4-(1-(trifluoromethyl)cyclopropyl)phenyl)carbamoyl)pyrrolidin-2-yl)pyridin-3-yl)benzoic acid

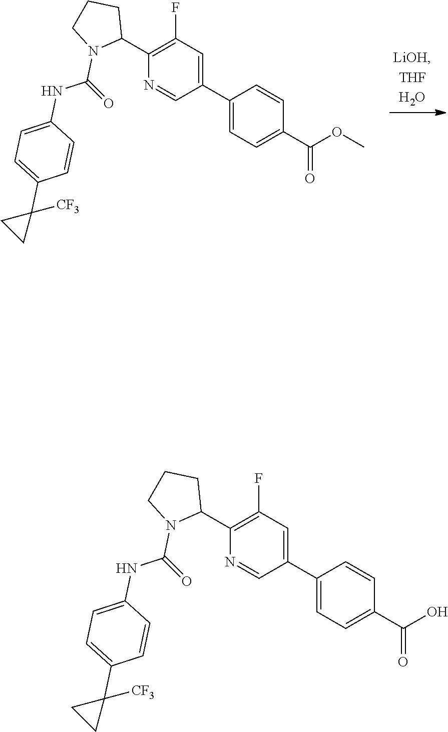 [SMILES: COC(=O)c1ccc(-c2cnc(C3CCCN3C(=O)Nc3ccc(C4(C(F)(F)F)CC4)cc3)c(F)c2)cc1 ; O=C(O)c1ccc(-c2cnc(C3CCCN3C(=O)Nc3ccc(C4(C(F)(F)F)CC4)cc3)c(F)c2)cc1]

To a stirred mixture of methyl 4-(5-fluoro-6-(1-((4-(1-(trifluoromethyl)cyclopropyl)phenyl)carbamoyl)pyrrolidin-2-yl)pyridin-3-yl)benzoate (125 mg, 0.2 mmol) in H₂O (0.5 mL) and THF (2 mL) was added LiOH monohydrate (50.4 mg, 1.2 mmol) in portions at room temperature. After 16 hours, the mixture was acidified to pH 6 with 1 M HCl (aq). The resulting mixture was extracted with EtOAc (3×20 mL). The combined organic layers were washed with brine, dried over anhydrous Na₂SO₄, filtered and concentrated under reduced pressure. The crude residue was purified by reversed-phase flash chromatography (conditions: column: C18 silica gel; mobile phase A: water (0.1% formic acid), Mobile Phase B: MeCN; Gradient (B %): 10% to 40% in 20 min; detector: UV 254 nm) to afford 4-(5-fluoro-6-(1-((4-(1-(trifluoromethyl)cyclopropyl)phenyl)carbamoyl)pyrrolidin-2-yl)pyridin-3-yl)benzoic acid (85 mg, 69% yield) as a white solid. LCMS (ESI) [M+H]⁺: 514

### Step 3: (S)-4-(5-fluoro-6-(1-((4-(1-(trifluoromethyl)cyclopropyl)phenyl)carbamoyl)pyrrolidin-2-yl)pyridin-3-yl)benzoic acid and (R)-4-(5-fluoro-6-(1-((4-(1-(trifluoromethyl)cyclopropyl)phenyl)carbamoyl)pyrrolidin-2-yl)pyridin-3-yl)benzoic acid

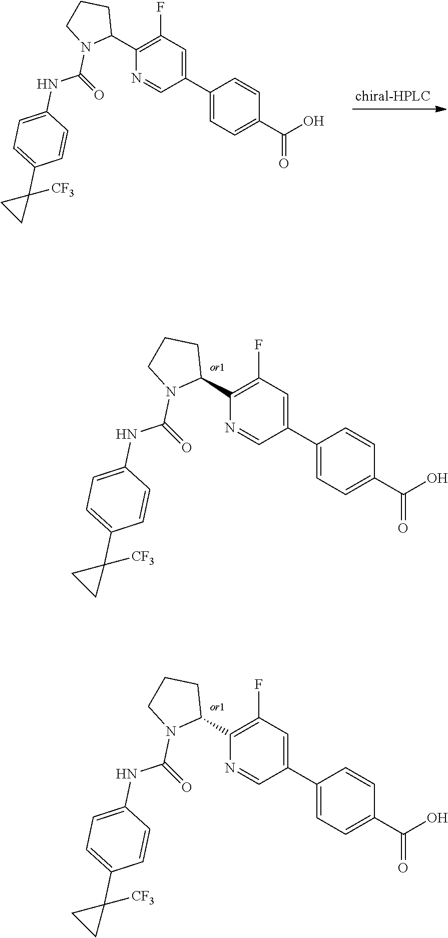 [SMILES: O=C(O)c1ccc(-c2cnc(C3CCCN3C(=O)Nc3ccc(C4(C(F)(F)F)CC4)cc3)c(F)c2)cc1 ; O=C(O)c1ccc(-c2cnc([C@@H]3CCCN3C(=O)Nc3ccc(C4(C(F)(F)F)CC4)cc3)c(F)c2)cc1 ; O=C(O)c1ccc(-c2cnc([C@H]3CCCN3C(=O)Nc3ccc(C4(C(F)(F)F)CC4)cc3)c(F)c2)cc1]

4-(5-fluoro-6-(1-((4-(1-(trifluoromethyl)cyclopropyl)phenyl)carbamoyl)pyrrolidin-2-yl)pyridin-3-yl)benzoic acid (83 mg) was purified by Prep-Chiral-HPLC (conditions: Column: CHIRALPAK IK 3*25 cm, 5 μm; Mobile Phase A: Hexane (0.1% formic acid), Mobile Phase B: EtOH; Flow rate: 40 mL/min; Gradient (B %): isocratic 30% B; Wave Length: 248/286 nm; RT1 (min): 6.42; RT2 (min): 10.44) to afford first peak (S)-4-(5-fluoro-6-(1-((4-(1-(trifluoromethyl)cyclopropyl)phenyl)carbamoyl)pyrrolidin-2-yl)pyridin-3-yl)benzoic acid (25.3 mg, Compound 44a; Method K, Peak 1, 0.82 min) as a white solid and second peak (R)-4-(5-fluoro-6-(1-((4-(1-(trifluoromethyl)cyclopropyl)phenyl)carbamoyl)pyrrolidin-2-yl)pyridin-3-yl)benzoic acid (25.1 mg, Compound 44b; Method K, Peak 2, 1.29 min) as a white solid.

| Cmpd # | Characterization Data |
|---|---|
| 44a | LCMS (ESI) [M + H]⁺: 514.2 |
| | ¹H NMR (400 MHz, DMSO-d₆) δ 13.07 (bs, 1H), 8.73 (d, J = 1.6 Hz, 1H), 8.34 (s, 1H), 8.10-7.99 |
| | (m, 3H), 7.91-7.84 (m, 2H), 7.51-7.41 (m, 2H), 7.25 (d, J = 8.4 Hz, 2H), 5.40-5.21 (m, 1H), |
| | 3.88-3.70 (m, 1H), 3.69-3.56 (m, 1H), 2.40-2.31 (m, 1H), 2.18-2.08 (m, 1H), 2.08-1.97 (m, |
| | 1H), 1.97-1.83 (m, 1H), 1.30-1.21 (m, 2H), 1.07-0.97 (m, 2H). |
| 44b | LCMS (ESI) [M + H]⁺: 514.2 |
| | ¹H NMR (400 MHz, DMSO-d₆) δ13.07 (bs, 1H), 8.73 (d, J = 1.6 Hz, 1H), 8.34 (s, 1H), 8.10-7.99 |
| | (m, 3H), 7.91-7.84 (m, 2H), 7.51-7.41 (m, 2H), 7.25 (d, J = 8.4 Hz, 2H), 5.40-5.21 (m, 1H), |
| | 3.88-3.70 (m, 1H), 3.69-3.56 (m, 1H), 2.40-2.31 (m, 1H), 2.18-2.08 (m, 1H), 2.08-1.97 (m, |
| | 1H), 1.97-1.83 (m, 1H), 1.30-1.21 (m, 2H), 1.07-0.97 (m, 2H). |

### Example 45: (S)-4-(5-fluoro6-(1-((2-fluoro-4-isopropylphenyl)carbamoyl)pyrrolidin-2-yl)pyridin-3-yl)benzoic acid and (R)-4-(5-fluoro-6-(1-((2-fluoro-4-isopropylphenyl)carbamoyl)pyrrolidin-2-yl)pyridin-3-yl)benzoic acid

### Step 1: 2-fluoro-4-(prop-1-en-2-yl)aniline

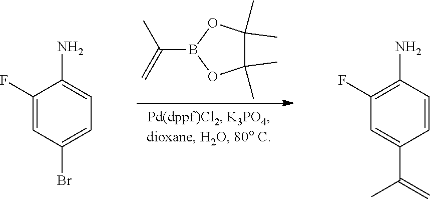

To a mixture of 4-bromo-2-fluoroaniline (2.0 g, 10.5 mmol), 4,4,5,5-tetramethyl-2-(prop-1-en-2-yl)-1,3,2-dioxaborolane (2.12 g, 12.6 mmol) and Pd(dppf)Cl₂ (0.77 g, 1.0 mmol) in dioxane (20 mL) and H₂O (4 mL) was added K₃PO₄ (6.7 g, 31.5 mmol) at room temperature under nitrogen atmosphere. The resulting mixture was stirred at 80° C. for 2 hours. Upon completion, the reaction was allowed to cool down to room temperature and was diluted with water. The resulting mixture was extracted with EtOAc (3×50 mL). The combined organic layers were washed with brine, dried over anhydrous Na₂SO₄, filtered and concentrated under reduced pressure. The crude residue was purified by silica gel column chromatography, eluted with PE/EA (7:1) to afford 2-fluoro-4-(prop-1-en-2-yl)aniline (1.2 g, 75% yield) as a light purple oil. LCMS (ESI) [M+H]⁺: 152

### Step 2: 2-fluoro-4-isopropylaniline

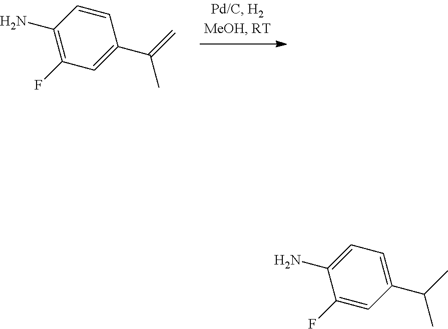

To a solution of 2-fluoro-4-(prop-1-en-2-yl)aniline (600 mg, 3.9 mmol) in MeOH (6 mL) was added Pd/C (84 mg, 0.08 mmol, 10 wt %) in portions at room temperature under nitrogen atmosphere. The resulting mixture was stirred at room temperature for 2 hours under hydrogen atmosphere. Upon completion, the resulting mixture was filtered over celite. The filter cake was washed with MeOH (3×5 mL). The filtrate was concentrated under reduced pressure to afford 2-fluoro-4-isopropylaniline (550 mg, crude) as a light purple oil, which was used in the next step directly without further purification. LCMS (ESI) [M+H]⁺: 154

### Step 3: methyl 4-(5-fluoro-6-(1-((2-fluoro-4-isopropylphenyl)carbamoyl)pyrrolidin-2-yl)pyridin-3-yl)benzoate

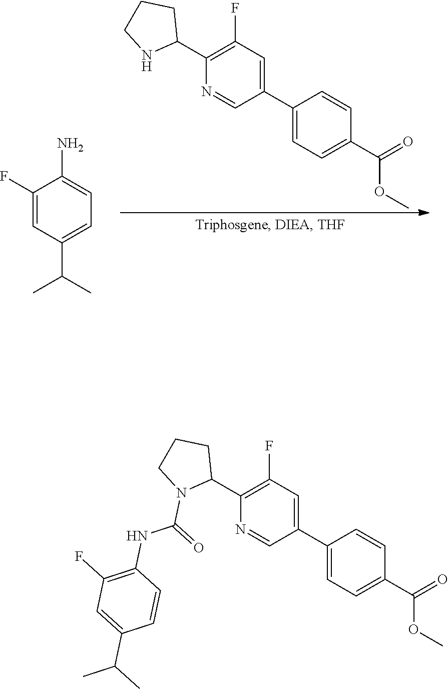 [SMILES: CC(C)c1ccc(N)c(F)c1 ; COC(=O)c1ccc(-c2cnc(C3CCCN3)c(F)c2)cc1 ; COC(=O)c1ccc(-c2cnc(C3CCCN3C(=O)Nc3ccc(C(C)C)cc3F)c(F)c2)cc1]

To a mixture of ditrichloromethyl carbonate (60 mg, 0.2 mmol) in THF (2 mL) was added 2-fluoro-4-isopropylaniline (77 mg, 0.5 mmol) and DIEA (323 mg, 2.5 mmol) dropwise at 0° C. under nitrogen atmosphere. After 10 minutes, methyl 4-(5-fluoro-6-(pyrrolidin-2-yl)pyridin-3-yl)benzoate (150 mg, 0.5 mmol) in THF (2 mL) was added dropwise over 2 minutes. The resulting mixture was warmed to room temperature and stirred for an additional 1 hour. Upon completion, the reaction was quenched with water at 0° C. The resulting mixture was extracted with EtOAc (3×20 mL). The combined organic layers were washed with brine, dried over anhydrous Na₂SO₄, filtered and concentrated under reduced pressure. The crude residue was purified by silica gel column chromatography, eluted with PE/EA (3:1) to afford methyl 4-(5-fluoro-6-(1-((2-fluoro-4-isopropylphenyl)carbamoyl)pyrrolidin-2-yl)pyridin-3-yl)benzoate (120 mg, 50% yield) as a light yellow solid. LCMS (ESI) [M+H]⁺: 480

### Step 4: 4-(5-fluoro-6-(1-((2-fluoro-4-isopropylphenyl)carbamoyl)pyrrolidin-2-yl)pyridin-3-yl)benzoic acid

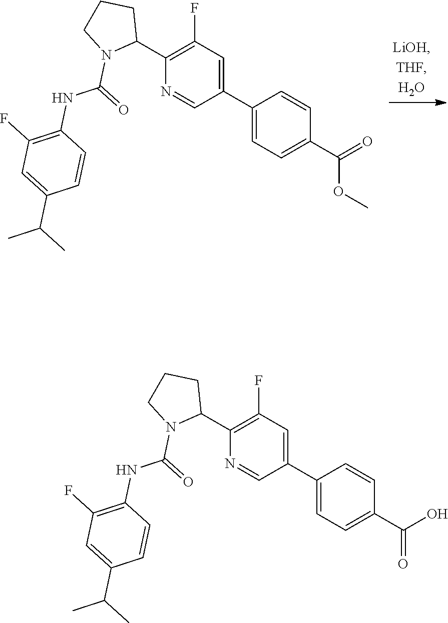 [SMILES: COC(=O)c1ccc(-c2cnc(C3CCCN3C(=O)Nc3ccc(C(C)C)cc3F)c(F)c2)cc1 ; CC(C)c1ccc(NC(=O)N2CCCC2c2ncc(-c3ccc(C(=O)O)cc3)cc2F)c(F)c1]

To a mixture of methyl 4-(5-fluoro-6-(1-((2-fluoro-4-isopropylphenyl)carbamoyl)pyrrolidin-2-yl)pyridin-3-yl)benzoate (120 mg, 0.2 mmol) in H₂O (0.5 mL) and THF (2 mL) was added LiOH monohydrate (50.4 mg, 1.2 mmol) in portions at room temperature. After 16 hours, the mixture was acidified to pH 6 with 1 M HCl (aq). The resulting mixture was extracted with EtOAc (3×20 mL). The combined organic layers were washed with brine, dried over anhydrous Na₂SO₄, filtered and concentrated under reduced pressure. The crude residue was purified by silica gel column chromatography, eluted with PE/EA (1:1) to afford 4-(5-fluoro-6-(1-((2-fluoro-4-isopropylphenyl)carbamoyl)pyrrolidin-2-yl)pyridin-3-yl)benzoic acid (90 mg, 77% yield) as a white solid. LCMS (ESI) [M+H]⁺: 466

### Step 5: (S)-4-(5-fluoro-6-(1-((2-fluoro-4-isopropylphenyl)carbamoyl)pyrrolidin-2-yl)pyridin-3-yl)benzoic acid and (R)-4-(5-fluoro-6-(1-((2-fluoro-4-isopropylphenyl)carbamoyl)pyrrolidin-2-yl)pyridin-3-yl)benzoic acid

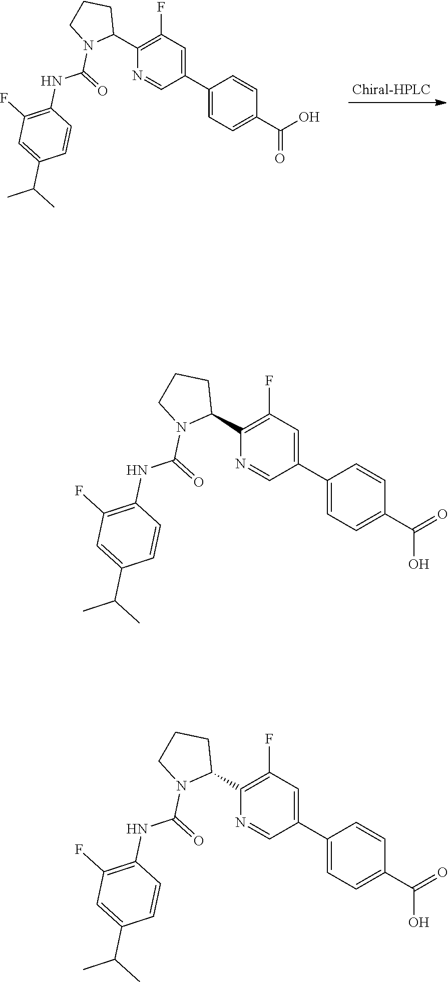 [SMILES: CC(C)c1ccc(NC(=O)N2CCCC2c2ncc(-c3ccc(C(=O)O)cc3)cc2F)c(F)c1 ; CC(C)c1ccc(NC(=O)N2CCC[C@H]2c2ncc(-c3ccc(C(=O)O)cc3)cc2F)c(F)c1 ; CC(C)c1ccc(NC(=O)N2CCC[C@@H]2c2ncc(-c3ccc(C(=O)O)cc3)cc2F)c(F)c1]

4-(5-fluoro-6-(1-((2-fluoro-4-isopropylphenyl)carbamoyl)pyrrolidin-2-yl)pyridin-3-yl)benzoic acid (90 mg) was purified by prep Chiral-HPLC (conditions: Column: CHIRAL ART Cellulose-SC, 3*25 cm, 5 μm; Mobile Phase A: Hexane (0.1% formic acid), Mobile Phase B: EtOH; Flow rate: 40 mL/min; Gradient (B %): isocratic 30% B; Wave Length: 240/250 nm; RT1 (min): 11.8; RT2 (min): 13.8) to afford first peak (S)-4-(5-fluoro-6-(1-((2-fluoro-4-isopropylphenyl)carbamoyl)pyrrolidin-2-yl)pyridin-3-yl)benzoic acid (39.6 mg, Compound 45a; Method A, Peak 1, 1.17 min) as a white solid and second peak (R)-4-(5-fluoro-6-(1-((2-fluoro-4-isopropylphenyl)carbamoyl)pyrrolidin-2-yl)pyridin-3-yl)benzoic acid (36.5 mg, Compound 45b; Method A, Peak 2, 1.48 min) as a white solid.

| Cmpd # | Characterization Data |
|---|---|
| 45a | LCMS (ESI) [M + H]⁺: 466.1 |
| | ¹H NMR (400 MHz, DMSO-d₆) δ 13.10 (s, 1H), 8.75 (d, J = 1.6 Hz, 1H), 8.16-7.98 (m, 3H), 7.97- |
| | 7.89 (m, 2H), 7.88-7.71 (m, 1H), 7.34 (t, J = 8.4 Hz, 1H), 7.09-6.98 (m, 1H), 6.98-6.85 (m, |
| | 1H), 5.41-5.29 (m, 1H), 3.81-3.70 (m, 1H), 3.70-3.59 (m, 1H), 2.90-2.78 (m, 1H), 2.41- |
| | 2.30 (m, 1H), 2.20-2.08 (m, 1H), 2.08-1.97 (m, 1H), 1.97-1.87 (m, 1H), 1.15 (d, J = 6.9 Hz, |
| | 6H). |
| 45b | LCMS (ESI) [M + H]⁺: 466.1 |
| | ¹H NMR (400 MHz, DMSO-d₆) δ 8.75 (d, J = 1.6 Hz, 1H), 8.16-7.98 (m, 3H), 7.97-7.89 (m, |
| | 2H), 7.88-7.71 (m, 1H), 7.34 (t, J = 8.4 Hz, 1H), 7.09-6.98 (m, 1H), 6.98-6.85 (m, 1H), 5.41- |
| | 5.29 (m, 1H), 3.81-3.70 (m, 1H), 3.70-3.59 (m, 1H), 2.90-2.78 (m, 1H), 2.41-2.30 (m, 1H), |
| | 2.20-2.08 (m, 1H), 2.08-1.97 (m, 1H), 1.97-1.87 (m, 1H), 1.15 (d, J = 6.9 Hz, 6H). |

### Example 46: (S)-4-(6-(1-((4-isopropylphenyl)carbamoyl)pyrrolidin-2-yl)-5-methylpyridin-3-yl)benzoic acid and (R)-4-(6-(1-((4-isopropylphenyl)carbamoyl)pyrrolidin-2-yl)-5-methylpyridin-3-yl)benzoic acid

### Step 1: tert-butyl 2-(5-chloro-3-methylpyridin-2-yl)pyrrolidine-1-carboxylate

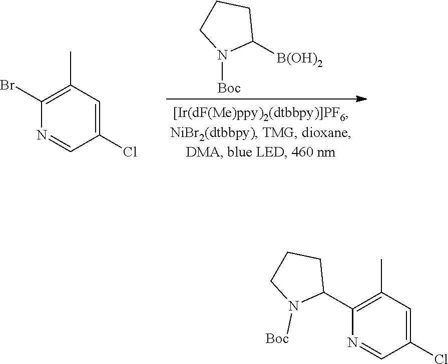

To a mixture of 2-bromo-5-chloro-3-methylpyridine (500 mg, 2.4 mmol), 1-(tert-butoxycarbonyl)pyrrolidin-2-ylboronic acid (781 mg, 3.6 mmol), [Ir(dF(Me)ppy)₂(dtbbpy)]PF₆ (61 mg, 61 μmol), N,N,N′,N′-tetramethylguanidine (836 mg, 7.2 mmol) and [4,4′-Bis(tert-butyl)-2,2′-bipyridine]nickel dibromide (117 mg, 0.2 mmol) in dioxane (8 mL) was added N,N-dimethylacetamide (2 mL) dropwise at room temperature under nitrogen atmosphere. The resulting mixture was stirred at room temperature for 16 hours with irradiation by 460 nm blue light. Upon completion, the reaction was quenched with water at room temperature. The resulting mixture was extracted with EtOAc (3×50 mL). The combined organic layers were washed with water (3×20 mL), dried over anhydrous Na₂SO₄, filtered and concentrated under reduced pressure. The crude residue was purified by silica gel column chromatography, eluted with PE/EA (6:1) to afford tert-butyl 2-(5-chloro-3-methylpyridin-2-yl)pyrrolidine-1-carboxylate (280 mg, 39% yield) as a light yellow oil. LCMS [M+H]⁺: 297

### Step 2: tert-butyl 2-(5-(4-(tert-butoxycarbonyl)phenyl)-3-methylpyridin-2-yl)pyrrolidine-1-carboxylate

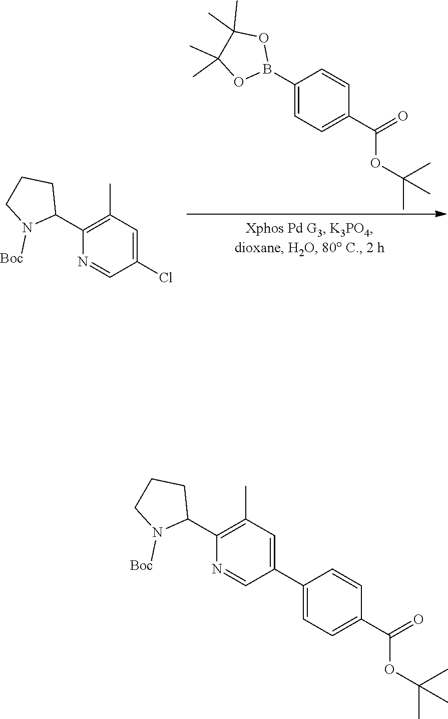 [SMILES: Cc1cc(Cl)cnc1C1CCCN1C(=O)OC(C)(C)C ; CC(C)(C)OC(=O)c1ccc(B2OC(C)(C)C(C)(C)O2)cc1 ; Cc1cc(-c2ccc(C(=O)OC(C)(C)C)cc2)cnc1C1CCCN1C(=O)OC(C)(C)C]

A solution of tert-butyl 2-(5-chloro-3-methylpyridin-2-yl)pyrrolidine-1-carboxylate (270 mg, 0.9 mmol), K₃PO₄ (579 mg, 2.7 mmol), Xphos Pd G₃ (77 mg, 91 μmol) and tert-butyl 4-(4,4,5,5-tetramethyl-1,3,2-dioxaborolan-2-yl) benzoate (332 mg, 1.0 mmol) in dioxane (4 mL) and H₂O (1 mL) was stirred at 80° C. for 2 hours under nitrogen atmosphere. The reaction was allowed to cool down to room temperature and was diluted with water. The resulting mixture was extracted with EtOAc (3×50 mL), dried over anhydrous Na₂SO₄, filtered and concentrated under reduced pressure. The crude residue was purified by silica gel column chromatography, eluted with PE/EA (2:1) to afford tert-butyl 2-(5-(4-(tert-butoxycarbonyl)phenyl)-3-methylpyridin-2-yl)pyrrolidine-1-carboxylate (200 mg, 50% yield) as a light yellow solid. LCMS [M+H]⁺: 439

### Step 3: 4-[5-methyl-6-(pyrrolidin-2-yl)pyridin-3-yl]benzoic acid

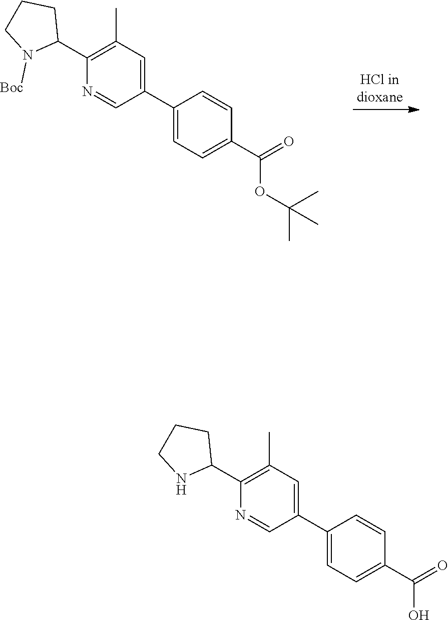 [SMILES: Cc1cc(-c2ccc(C(=O)OC(C)(C)C)cc2)cnc1C1CCCN1C(=O)OC(C)(C)C ; Cc1cc(-c2ccc(C(=O)O)cc2)cnc1C1CCCN1]

A solution of tert-butyl 2-(5-(4-(tert-butoxycarbonyl)phenyl)-3-methylpyridin-2-yl)pyrrolidine-1-carboxylate (190 mg, 0.4 mmol) in 4.0 M HCl in 1,4-dioxane (2 mL) was stirred at room temperature for 4 hours. Upon completion, the resulting mixture was concentrated under reduced pressure to afford 4-[5-methyl-6-(pyrrolidin-2-yl)pyridin-3-yl]benzoic acid (120 mg, crude), which was used directly in the next step without further purification LCMS [M+H]⁺: 283

### Step 4: 4-(6-(1-((4-isopropylphenyl)carbamoyl)pyrrolidin-2-yl)-5-methylpyridin-3-yl)benzoic acid

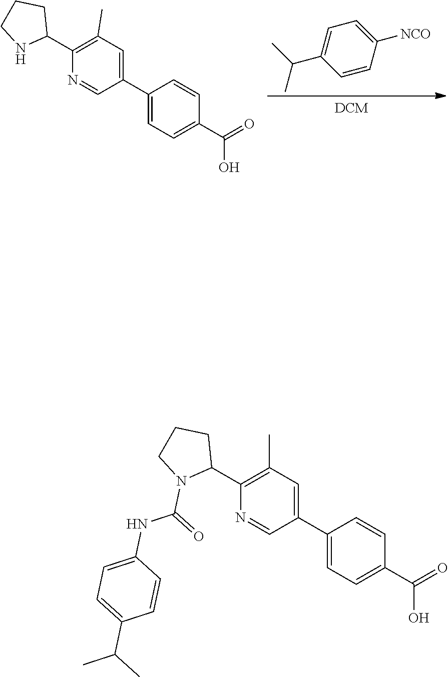

A solution of 4-[5-methyl-6-(pyrrolidin-2-yl)pyridin-3-yl]benzoic acid (110 mg, 0.3 mmol) and 1-isocyanato-4-isopropylbenzene (62 mg, 0.3 mmol) in DCM (2 mL) was stirred at room temperature for 2 hours. Upon completion, the resulting mixture was concentrated under reduced pressure. The crude residue was purified by reversed-phase flash chromatography (conditions: column: C18 silica gel; mobile phase A water (0.1% formic acid), Mobile phase B: MeCN; Gradient (B %): 30% to 80% in 20 min; detector, UV 254 nm) to afford 4-(6-(1-((4-isopropylphenyl)carbamoyl)pyrrolidin-2-yl)-5-methylpyridin-3-yl)benzoic acid (90 mg, 52% yield) as an off-white solid. LCMS [M+H]⁺: 444

### Step 5: (S)-4-(6-(1-((4-isopropylphenyl)carbamoyl)pyrrolidin-2-yl)-5-methylpyridin-3-yl)benzoic acid and (R)-4-(6-(1-((4-isopropylphenyl)carbamoyl)pyrrolidin-2-yl)-5-methylpyridin-3-yl)benzoic acid

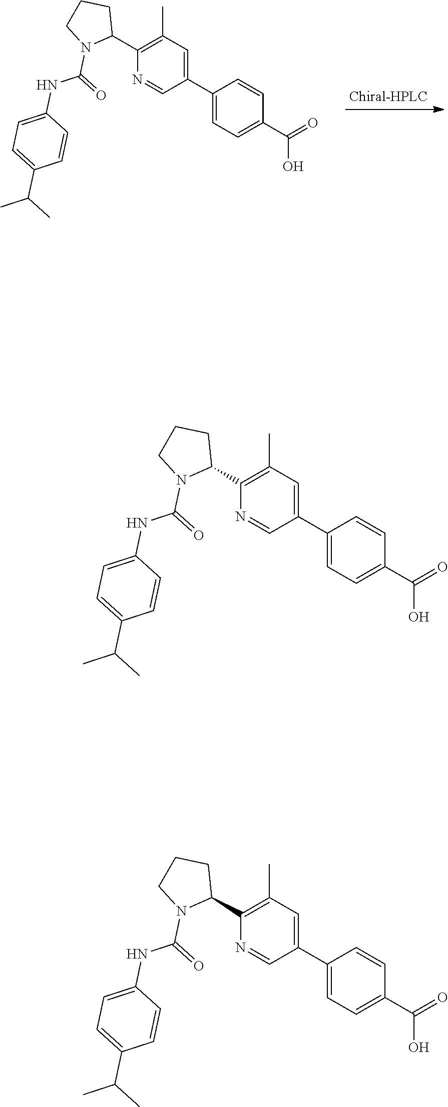 [SMILES: Cc1cc(-c2ccc(C(=O)O)cc2)cnc1C1CCCN1C(=O)Nc1ccc(C(C)C)cc1 ; Cc1cc(-c2ccc(C(=O)O)cc2)cnc1[C@H]1CCCN1C(=O)Nc1ccc(C(C)C)cc1 ; Cc1cc(-c2ccc(C(=O)O)cc2)cnc1[C@@H]1CCCN1C(=O)Nc1ccc(C(C)C)cc1]

4-(6-(1-((4-isopropylphenyl)carbamoyl)pyrrolidin-2-yl)-5-methylpyridin-3-yl)benzoic acid (90 mg, 0.2 mmol) was purified by prep-Chiral-HPLC (conditions: Column: CHIRALPAK IK 3*25 cm, 5 μm; Mobile Phase A: Hexane (0.1% formic acid), Mobile Phase B: EtOH; Flow rate: 40 mL/min; Gradient (B %): isocratic 20% B; Wave Length: 262/236 nm; RT1 (min): 14.2; RT2 (min): 17) to afford first peak (S)-4-(6-(1-((4-isopropylphenyl)carbamoyl)pyrrolidin-2-yl)-5-methylpyridin-3-yl)benzoic acid (32.1 mg, Compound 46a; Method K, Peak 1, 0.96 min) as an off-white solid and second peak (R)-4-(6-(1-((4-isopropylphenyl)carbamoyl)pyrrolidin-2-yl)-5-methylpyridin-3-yl)benzoic acid (31.0 mg, Compound 46b, Method K, Peak 2, 1.22 min) as a white solid.

| Cmpd # | Characterization Data |
|---|---|
| 46a | LCMS (ESI) [M + H]⁺: 444.2 |
| | ¹H NMR (400 MHz, DMSO-d₆) δ 12.99 (bs, 1H), 8.66 (d, J = 2.3 Hz, 1H), 8.12-7.97 (m, 3H), |
| | 7.88-7.79 (m, 3H), 7.39-7.25 (m, 2H), 7.09-6.81 (m, 2H), 5.41-5.13 (m, 1H), 3.87-3.74 (m, |
| | 1H), 3.68-3.59 (m, 1H), 2.82-2.71 (m, 1H), 2.47 (s, 3H), 2.35-2.25 (m, 1H), 2.19-2.08 (m, |
| | 1H), 2.04-1.90 (m, 1H), 1.87-1.71 (m, 1H), 1.14 (d, J = 6.9 Hz, 6H). |
| 46b | LCMS (ESI) [M + H]⁺: 444.2 |
| | ¹H NMR (400 MHz, DMSO-d₆) δ 12.99 (bs, 1H), 8.66 (d, J = 2.3 Hz, 1H), 8.12-7.97 (m, 3H), |
| | 7.87-7.79 (m, 3H), 7.39-7.25 (m, 2H), 7.09-6.81 (m, 2H), 5.41-5.13 (m, 1H), 3.87-3.74 (m, |
| | 1H), 3.68-3.59 (m, 1H), 2.82-2.71 (m, 1H), 2.47 (s, 3H), 2.35-2.25 (m, 1H), 2.19-2.08 (m, |
| | 1H), 2.04-1.90 (m, 1H), 1.87-1.71 (m, 1H), 1.14 (d, J = 6.9 Hz, 6H). |

### Example 47: (S)-4′-(1-((4-isopropylphenyl)carbamoyl)pyrrolidin-2-yl)-[1,1′-biphenyl]-4-carboxylic acid

### Step 1: tert-butyl (S)-2-(4′-(tert-butoxycarbonyl)-[1,1′-biphenyl]-4-yl)pyrrolidine-1-carboxylate

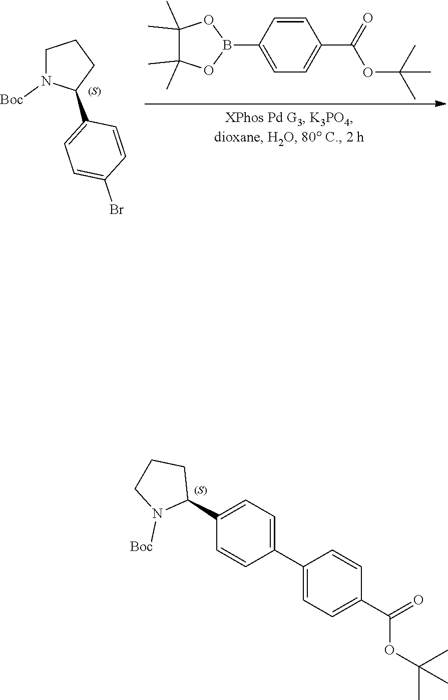

A mixture of tert-butyl (S)-2-(4-bromophenyl)pyrrolidine-1-carboxylate (293 mg, 0.9 mmol), tert-butyl 4-(4,4,5,5-tetramethyl-1,3,2-dioxaborolan-2-yl)benzoate (395 mg, 1.3 mmol), Xphos Pd G₃ (77 mg, 92 μmol) and K₃PO₄ (585 mg, 2.7 mmol) in dioxane (5 mL) and H₂O (1 mL) was stirred at 80° C. for 2 hours under nitrogen atmosphere. Upon completion, the mixture was allowed to cool down to room temperature. The resulting mixture was concentrated under reduced pressure. The crude residue was directly purified by silica gel column chromatography, eluted with PE/EA (1:1) to afford tert-butyl (S)-2-(4′-(tert-butoxycarbonyl)-[1,1′-biphenyl]-4-yl)pyrrolidine-1-carboxylate (310 mg, 79% yield) as a white solid. LCMS [M+H]⁺: 424

### Step 2: (S)-4′-(pyrrolidin-2-yl)-[1,1′-biphenyl]-4-carboxylic acid

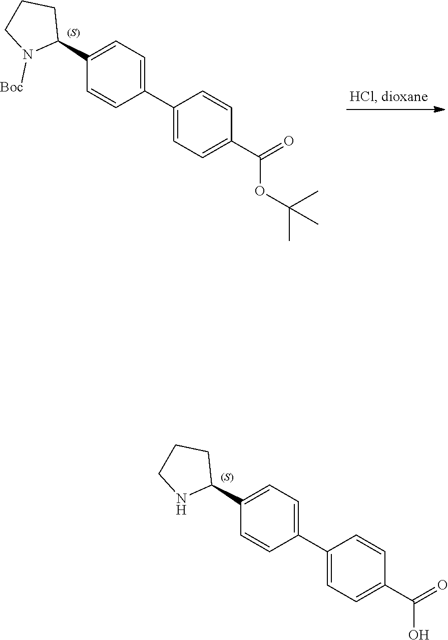

A solution of tert-butyl (S)-2-(4′-(tert-butoxycarbonyl)-[1,1′-biphenyl]-4-yl)pyrrolidine-1-carboxylate (300 mg, 0.7 mmol) in 4 M HCl in dioxane (10 mL) was stirred at room temperature for 1 hour. Upon completion, the resulting mixture was concentrated under reduced pressure to afford (S)-4′-(pyrrolidin-2-yl)-[1,1′-biphenyl]-4-carboxylic acid (180 mg, crude) as a white solid, which was used directly in the next step without further purification. LCMS [M+H]⁺: 268

### Step 3: (S)-4′-(1-((4-isopropylphenyl)carbamoyl)pyrrolidin-2-yl)-[1,1′-biphenyl]-4-carboxylic acid

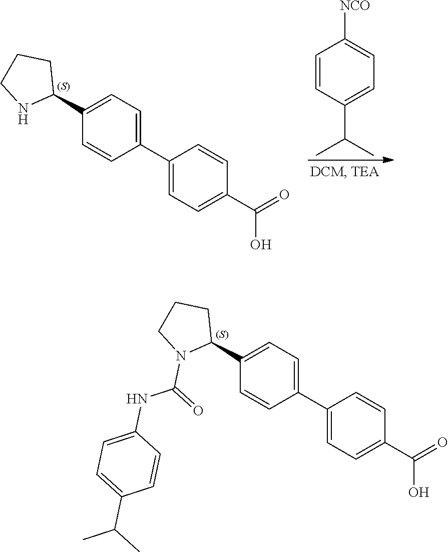

To a stirred solution of (S)-4′-(pyrrolidin-2-yl)-[1,1′-biphenyl]-4-carboxylic acid (80 mg, 0.3 mmol) and TEA (151 mg, 1.4 mmol) in DCM (1 mL) was added 1-isocyanato-4-isopropylbenzene (48 mg, 0.3 mmol) in portions at 0° C. The resulting mixture was warmed to room temperature and stirred for an additional 2 hours. Upon completion, the resulting mixture was concentrated under reduced pressure. The crude residue was directly purified by prep Prep-HPLC (conditions; Column: Xselect CSH Prep C18, 30*150 mm 5 μm; Mobile Phase A: Water (0.1% formic acid), Mobile Phase B: ACN; Flow rate: 60 mL/min; Gradient (B %): 48% to 68% in 10 min; Wave Length: 254 nm/220 nm; RT1 (min): 10.57) to afford (S)-4′-(1-((4-isopropylphenyl)carbamoyl)pyrrolidin-2-yl)-[1,1′-biphenyl]-4-carboxylic acid (36.0 mg, Compound 47; Method I; Peak 1, 1.51 min—analyzed against Compound 48) as a white solid.

| Cmpd # | Characterization Data |
|---|---|
| 47 | LCMS (ESI) [M + H]⁺: 429.2 |
| | ¹H NMR (400 MHz, DMSO-d₆) δ 12.95 (bs, 1H), 8.07 (s, 1H), 8.01-7.89 (m, 2H), 7.89-7.71 (m, |
| | 2H), 7.71-7.55 (m, 2H), 7.49-7.20 (m, 4H), 7.19-6.91 (m, 2H), 5.20-5.09 (m, 1H), 3.91-3.69 |
| | (m, 1H), 3.69-3.48 (m, 1H), 2.86-2.72 (m, 1H), 2.40-2.19 (m, 1H), 2.09-1.66 (m, 3H), 1.14 |
| | (d, J = 6.9 Hz, 6H). |

### Example 48: (R)-4′-(1-((4-isopropylphenyl)carbamoyl)pyrrolidin-2-yl)-[1,1′-biphenyl]-4-carboxylic acid

### Step 1: tert-butyl (R)-2-(4′-(tert-butoxycarbonyl)-[1,1′-biphenyl]-4-yl)pyrrolidine-1-carboxylate

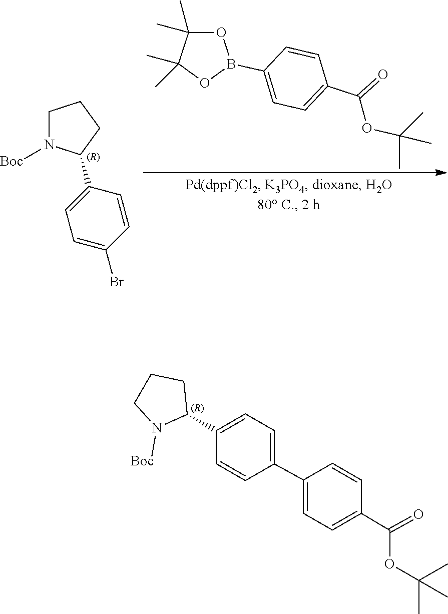

A mixture of tert-butyl (2R)-2-(4-bromophenyl)pyrrolidine-1-carboxylate (300 mg, 1.0 mmol), tert-butyl 4-(4,4,5,5-tetramethyl-1,3,2-dioxaborolan-2-yl)benzoate (279 mg, 1.0 mmol), Pd(dppf)Cl₂ (67 mg, 0.1 mmol) and K₃PO₄ (585 mg, 2.7 mmol) in dioxane (3 mL) and H₂O (0.6 mL) was stirred at 80° C. for 2 hours under nitrogen atmosphere. Upon completion, the mixture was allowed to cool down to room temperature and was diluted with water. The resulting mixture was extracted with EtOAc (3×100 mL). the combined organic layers were dried over anhydrous Na₂SO₄, filtered and concentrated under reduced pressure. The crude residue was purified by silica gel column chromatography, eluted with PE/EA (6:1) to afford tert-butyl (R)-2-(4′-(tert-butoxycarbonyl)-[1,1′-biphenyl]-4-yl)pyrrolidine-1-carboxylate (330 mg, 84% yield) as a white solid. LCMS (ESI) [M+H]⁺: 424

### Step 2: (R)-4′-(pyrrolidin-2-yl)-[1,1′-biphenyl]-4-carboxylic acid

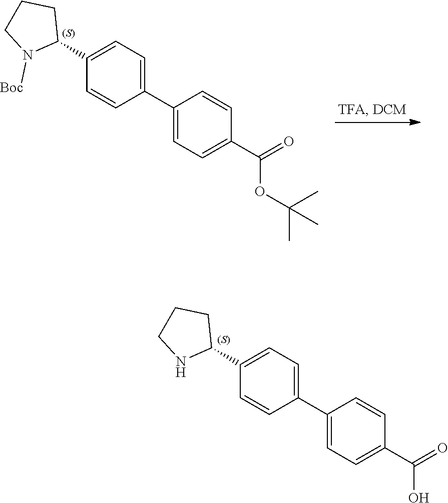

A mixture of tert-butyl (R)-2-(4′-(tert-butoxycarbonyl)-[1,1′-biphenyl]-4-yl)pyrrolidine-1-carboxylate (330 mg, 0.7 mmol) in TFA (1 mL) and DCM (3 mL) was stirred at room temperature for 6 hours. Upon completion, the resulting mixture was concentrated under reduced pressure to afford (R)-4′-(pyrrolidin-2-yl)-[1,1′-biphenyl]-4-carboxylic acid (200 mg, crude) as a white solid, which was used directly in the next step without further purification. LCMS (ESI) [M+H]⁺: 268

### Step 3: (R)-4′-(1-((4-isopropylphenyl)carbamoyl)pyrrolidin-2-yl)-[1,1′-biphenyl]-4-carboxylic acid

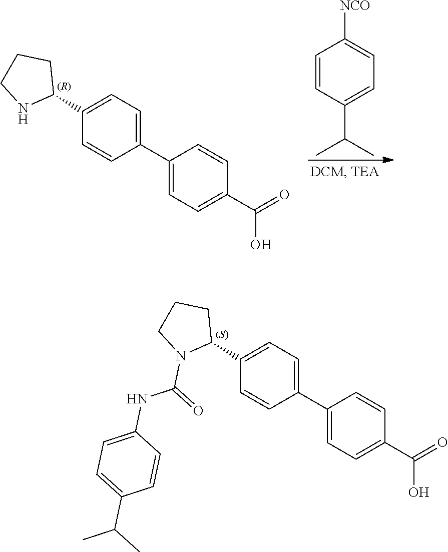

A mixture of (R)-4′-(pyrrolidin-2-yl)-[1,1′-biphenyl]-4-carboxylic acid (80 mg, 0.3 mmol), TEA (90 mg, 0.9 mmol) and 1-isocyanato-4-isopropylbenzene (48 mg, 0.3 mmol) in DCM (2 mL) was stirred at room temperature for 1 hour. Upon completion, the resulting mixture was diluted with water and extracted with EtOAc (3×50 mL). The combined organic layers were dried over anhydrous Na₂SO₄, filtered and concentrated under reduced pressure. The crude residue was purified by Prep-HPLC (conditions: Column: Xselect CSH Prep C18, 30*150 mm 5 m; Mobile Phase A: Water (0.1% formic acid), Mobile Phase B: ACN; Flow rate: 60 mL/min; Gradient (B %): 49% to 69% in 10 min; Wave Length: 254 nm/220 nm; RT1 (min): 10.25) to afford (R)-4′-(1-((4-isopropylphenyl)carbamoyl)pyrrolidin-2-yl)-[1,1′-biphenyl]-4-carboxylic acid (35.6 mg, Compound 48; Method I; Peak 2, 1.93 min—analyzed against Compound 47) as a white solid.

| Compound# | Characterization Data |
|---|---|
| 48 | LCMS (ESI) [M + H]⁺: 429.1 |
| | ¹H NMR (400 MHz, DMSO-d₆) δ 12.96 (bs, 1H), 8.08 (s, 1H), 8.05-7.99 (m, 2H), 7.80-7.71 |
| | (m, 2H), 7.71-7.63 (m, 2H), 7.43-7.27 (m, 4H), 7.10-7.01 (m, 2H), 5.22-5.01 (m, 1H), |
| | 3.89-3.73 (m, 1H), 3.65-3.52 (m, 1H), 2.83-2.70 (m, 1H), 2.39-2.28 (m, 1H), 2.01-1.83 (m, |
| | 2H), 1.83-1.73 (m, 1H), 1.15 (d, J = 6.9 Hz, 6H). |

### Example 49: (S)-4-(5-fluoro-6-(1-((4-isopropyl-3-methylphenyl)carbamoyl)pyrrolidin-2-yl)pyridin-3-yl)benzoic acid and (R)-4-(5-fluoro-6-(1-((4-isopropyl-3-methylphenyl)carbamoyl) pyrrolidin-2-yl)pyridin-3-yl)benzoic acid

### Step 1: 3-methyl-4-(prop-1-en-2-yl)aniline

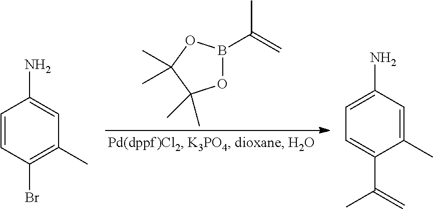

A solution of 4-bromo-3-methylaniline (1.00 g, 5.3 mmol), 4,4,5,5-tetramethyl-2-(prop-1-en-2-yl)-1,3,2-dioxaborolane (1.81 g, 10.7 mmol), Pd(dppf)Cl₂ (390 mg, 0.5 mmol), K₃PO₄ (3.42 g, 16.1 mmol) and H₂O (1 mL) in dioxane (10 mL) was stirred at 80° C. for 2 hours under nitrogen atmosphere. Upon completion, the mixture was allowed to cool down to room temperature. The resulting mixture was filtered. The filter cake was washed with DCM (3×15 mL). The filtrate was concentrated under reduced pressure. The crude residue was purified by silica gel column chromatography, eluted with PE/EA (1:1) to afford 3-methyl-4-(prop-1-en-2-yl)aniline (600 mg, 75% yield) as a yellow oil. LCMS [M+H]⁺: 148

### Step 2: 4-isopropyl-3-methylaniline

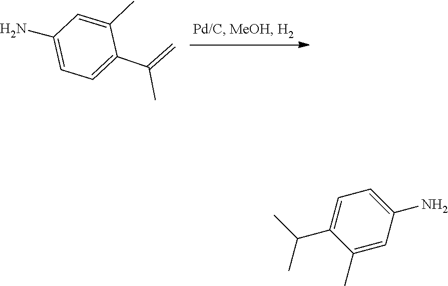

To a mixture of 3-methyl-4-(prop-1-en-2-yl)aniline (590 mg, 4.0 mmol) in MeOH (10 mL) was added Pd/C (426 mg, 10 wt %) under nitrogen atmosphere. The resulting mixture was stirred at room temperature for 4 hours under hydrogen atmosphere. Upon completion, the resulting mixture was filtered over celite. The filter cake was washed with MeOH (3×15 mL). The resulting mixture was concentrated under reduced pressure. The crude residue was purified by silica gel column chromatography, eluted with PE/EA (1:1) to afford 4-isopropyl-3-methylaniline (450 mg, 75% yield) as a yellow oil. LCMS [M+H]⁺: 150

### Step 3: 4-(5-fluoro-6-(1-((4-isopropyl-3-methylphenyl)carbamoyl)pyrrolidin-2-yl)pyridin-3-yl)benzoic acid

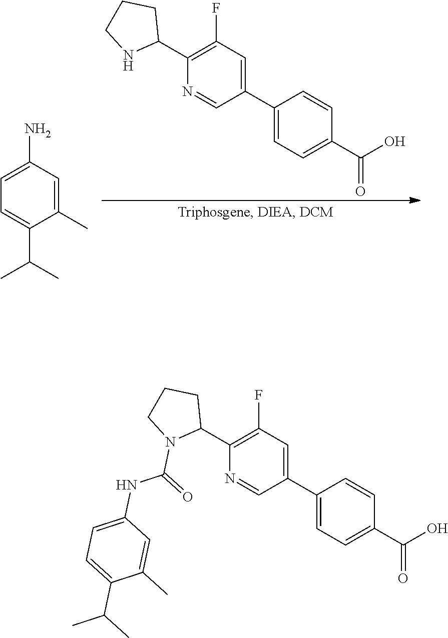 [SMILES: Cc1cc(N)ccc1C(C)C ; O=C(O)c1ccc(-c2cnc(C3CCCN3)c(F)c2)cc1 ; Cc1cc(NC(=O)N2CCCC2c2ncc(-c3ccc(C(=O)O)cc3)cc2F)ccc1C(C)C]

To a mixture of 4-isopropyl-3-methylaniline (80 mg, 0.5 mmol) in DCM (5 mL) were added with DIEA (346 mg, 2.7 mmol) and ditrichloromethyl carbonate (80 mg, 0.3 mmol) dropwise at 0° C. under nitrogen atmosphere. After 30 minutes, the reaction was warmed to room temperature and 4-(5-fluoro-6-(pyrrolidin-2-yl)pyridin-3-yl)benzoic acid (153 mg, 0.5 mmol) was added dropwise over 2 minutes. The resulting mixture was stirred for additional 2 hours. Upon completion, the reaction was quenched with ice water at 0° C. The resulting mixture was extracted with EtOAc (3×7 mL). The combined organic layers were dried over anhydrous Na₂SO₄, filtered and concentrated under reduced pressure. The crude residue was purified by silica gel column chromatography, eluted with CH₂Cl₂/MeOH (10:1) to afford 4-(5-fluoro-6-(1-((4-isopropyl-3-methylphenyl)carbamoyl)pyrrolidin-2-yl)pyridin-3-yl)benzoic acid (80 mg, 32% yield) as a yellow solid. LCMS [M+H]⁺: 462

### Step 4: (S)-4-(5-fluoro-6-(1-((4-isopropyl-3-methylphenyl)carbamoyl)pyrrolidin-2-yl)pyridin-3-yl)benzoic acid and (R)-4-(5-fluoro-6-(1-((4-isopropyl-3-methylphenyl)carbamoyl)pyrrolidin-2-yl)pyridin-3-yl)benzoic acid

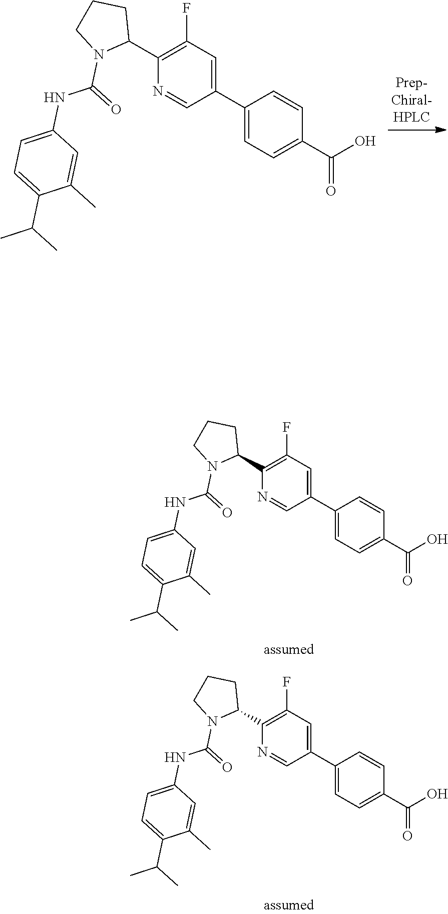 [SMILES: Cc1cc(NC(=O)N2CCCC2c2ncc(-c3ccc(C(=O)O)cc3)cc2F)ccc1C(C)C ; Cc1cc(NC(=O)N2CCC[C@H]2c2ncc(-c3ccc(C(=O)O)cc3)cc2F)ccc1C(C)C ; Cc1cc(NC(=O)N2CCC[C@@H]2c2ncc(-c3ccc(C(=O)O)cc3)cc2F)ccc1C(C)C]

4-(5-fluoro-6-(1-((4-isopropyl-3-methylphenyl)carbamoyl)pyrrolidin-2-yl)pyridin-3-yl)benzoic acid (78 mg) was separated by Prep-Chiral HPLC (conditions: Column: CHIRALPAK IK 3*25 cm, 5 μm; Mobile Phase A: Hexane (0.1% formic acid), Mobile Phase B: EtOH; Flow rate: 40 mL/min; Gradient (B %): isocratic 25% B; Wave Length: 247/210 nm; RT1 (min): 6.79; RT2 (min): 15.15) to afford first peak (S)-4-(5-fluoro-6-(1-((4-isopropyl-3-methylphenyl)carbamoyl)pyrrolidin-2-yl)pyridin-3-yl)benzoic acid (20.5 mg, Compound 49a; Method P, Peak 1, 1.53 min) as a white solid and second peak (R)-4-(5-fluoro-6-(1-((4-isopropyl-3-methylphenyl)carbamoyl)pyrrolidin-2-yl)pyridin-3-yl)benzoic acid (26.3 mg, Compound 49b; Method P, Peak 2, 2.77 min) as a white solid.

| Cmpd # | Characterization Data |
|---|---|
| 49a | LCMS (ESI) [M + H]⁺: 462.2 |
| | ¹H NMR (400 MHz, DMSO-d₆) δ 13.09 (bs, 1H), 8.73 (t, J = 1.6 Hz, 1H), 8.08-8.02 (m, 4H), |
| | 7.90-7.88 (m, 2H), 7.21 (d, J = 2.4 Hz, 2H), 7.03-7.01 (m, 1H), 5.33 (d, J = 12 Hz, 1H), 3.76- |
| | 3.74 (m, 1H), 3.64-3.60 (m, 1H), 3.00-2.97 (m, 1H), 2.36-2.32 (m, 1H), 2.19 (s, 3H), 2.11- |
| | 2.10 (m, 1H), 2.03-1.99 (m, 1H), 1.91-1.88 (m, 1H), 1.11 (d, J = 6.8 Hz, 6H). |
| 49b | LCMS (ESI) [M + H]⁺: 462.2 |
| | ¹H NMR (400 MHz, DMSO-d₆) δ 13.09 (bs, 1H), 8.73 (t, J = 1.6 Hz, 1H), 8.08-8.02 (m, 4H), |
| | 7.90-7.88 (m, 2H), 7.21 (d, J = 2.4 Hz, 2H), 7.03-7.01 (m, 1H), 5.33 (d, J = 12 Hz, 1H), 3.76- |
| | 3.74 (m, 1H), 3.64-3.60 (m, 1H), 3.00-2.97 (m, 1H), 2.36-2.32 (m, 1H), 2.19 (s, 3H), 2.11- |
| | 2.10 (m, 1H), 2.03-1.99 (m, 1H), 1.91-1.88 (m, 1H), 1.11 (d, J = 6.8 Hz, 6H). |

### Example 51: (R)-3-fluoro-4′-(2-(((4-isopropylphenyl)carbamoyl)oxy)-3-methylbutanamido)-[1,1′-biphenyl]-4-carboxylic acid

### Step 1: tert-butyl (R)-3-fluoro-4′-(2-hydroxy-3-methylbutanamido)-[1,1′-biphenyl]-4-carboxylate

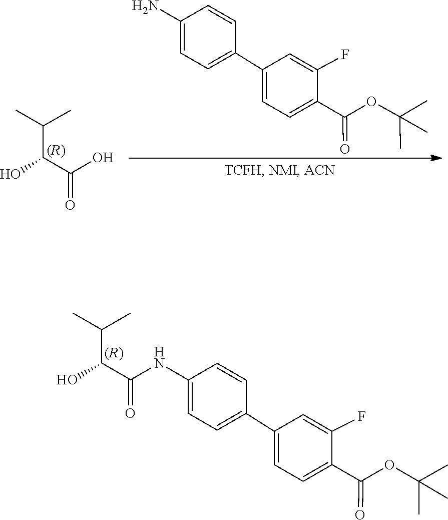

A solution of (R)-2-hydroxy-3-methylbutanoic acid (150 mg, 1.3 mmol), tert-butyl 4′-amino-3-fluoro-[1,1′-biphenyl]-4-carboxylate (365 mg, 1.3 mmol), N-(chloro(dimethylamino)methylene)-N-methylmethanaminium hexafluorophosphate(V) (356 mg, 1.3 mmol) and 1-methyl-1H-imidazole (521 mg, 6.4 mmol) in ACN (5 mL) was stirred for 2 hours at room temperature. Upon completion, the resulting mixture was concentrated under reduced pressure. The crude residue was purified by reversed-phase flash chromatography (conditions: column: C18 silica gel; mobile phase A: water (10 mmol/L NH₄HCO₃), Mobile Phase B: MeCN; Gradient (B %): 5% to 95% in 25 min; detector, UV 254 nm) to afford tert-butyl (R)-3-fluoro-4′-(2-hydroxy-3-methylbutanamido)-[1,1′-biphenyl]-4-carboxylate (140 mg, 28% yield) as a light yellow solid. LCMS (ESI) [M+H]⁺: 388

### Step 2: tert-butyl (R)-3-fluoro-4′-(2-(((4-isopropylphenyl)carbamoyl)oxy)-3-methylbutanamido)-[1,1′-biphenyl]-4-carboxylate

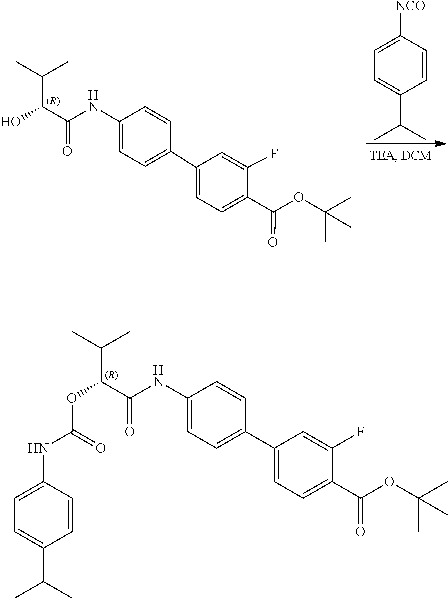

To a mixture of tert-butyl (R)-3-fluoro-4′-(2-hydroxy-3-methylbutanamido)-[1,1′-biphenyl]-4-carboxylate (130 mg, 0.3 mmol) in DCM (6 mL) were added TEA (85 mg, 0.8 mmol) and 1-isocyanato-4-isopropylbenzene (81 mg, 0.5 mmol) dropwise at 0° C. The resulting mixture was stirred at room temperature for 2 hours under nitrogen atmosphere. Upon completion, the reaction was quenched with ice water at 0° C. The resulting mixture was extracted with EtOAc (3×8 mL). The combined organic layers were dried over anhydrous Na₂SO₄, filtered and concentrated under reduced pressure. The crude residue was purified by silica gel column chromatography, eluted with PE/EA (3:1) to afford tert-butyl (R)-3-fluoro-4′-(2-(((4-isopropylphenyl)carbamoyl)oxy)-3-methylbutanamido)-[1,1′-biphenyl]-4-carboxylate (100 mg, 54% yield) as a white solid. LCMS (ESI) [M+H]⁺: 549

### Step 3: (R)-3-fluoro-4′-(2-(((4-isopropylphenyl)carbamoyl)oxy)-3-methylbutanamido)-[1,1′-biphenyl]-4-carboxylic acid

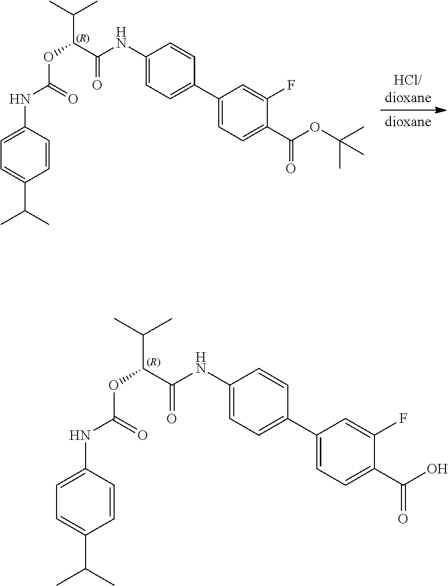

To a mixture of tert-butyl (R)-3-fluoro-4′-(2-(((4-isopropylphenyl)carbamoyl)oxy)-3-methylbutanamido)-[1,1′-biphenyl]-4-carboxylate (100 mg, 0.2 mmol) in dioxane (1 mL) was added 4M HCl in 1,4-dioxane (5 mL) dropwise at 0° C. The resulting mixture was stirred at room temperature for 2 hours. Upon completion, the resulting mixture was concentrated under reduced pressure. The crude residue was purified by Prep-HPLC (conditions: Column: XSelect CSH Fluoro Phenyl 30*150 mm, 5 μm; Mobile Phase A: Water (0.1% formic acid), Mobile Phase B: ACN; Flow rate: 60 mL/min; Gradient (B %): 41% to 62% in 15 min; Wave Length: 254 nm/220 nm; RT1 (min): 9.87) to afford (R)-3-fluoro-4′-(2-(((4-isopropylphenyl)carbamoyl)oxy)-3-methylbutanamido)-[1,1′-biphenyl]-4-carboxylic acid (35.4 mg, Compound 51; Method E, Peak 1, 1.27 min—analyzed against Compound 52) as a white solid.

| Cmpd # | Characterization Data |
|---|---|
| 51 | LCMS (ESI) [M + H]⁺: 493.2 |
| | ¹H NMR (400 MHz, DMSO-d₆) δ 13.16 (bs, 1H), 10.30 (s, 1H), 9.74 (bs, 1H), 7.93 (d, J = 8.0 Hz, |
| | 1H), 7.79-7.78 (m, 4H), 7.63-7.60 (m, 2H), 7.38 (d, J = 8.4 Hz, 2H), 7.15 (d, J = 8.4 Hz, 2H), |
| | 4.83 (d, J = 5.6 Hz, 1H), 2.84-2.82 (m, 1H), 2.23-2.18 (m, 1H), 1.16 (d, J = 6.8 Hz, 6H), 1.02 |
| | (d, J = 6.8 Hz, 6H). |

### Example 52: (S)-3-fluoro-4′-(2-(((4-isopropylphenyl)carbamoyl)oxy)-3-methylbutanamido)-[1,1′-biphenyl]-4-carboxylic acid

### Step 1: tert-butyl (S)-3-fluoro-4′-(2-hydroxy-3-methylbutanamido)-[1,1′-biphenyl]-4-carboxylate

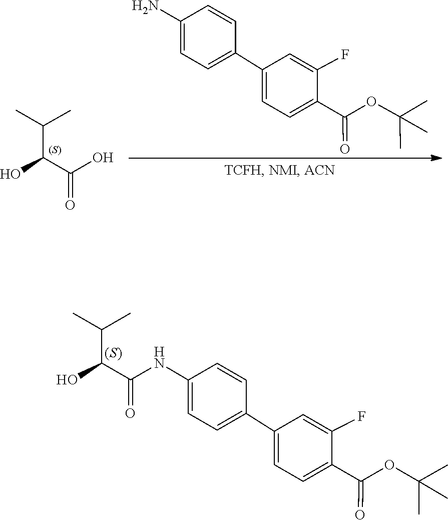

A solution of (S)-2-hydroxy-3-methylbutanoic acid (150 mg, 1.3 mmol), tert-butyl 4′-amino-3-fluoro-[1,1′-biphenyl]-4-carboxylate (365 mg, 1.3 mmol), N-(chloro(dimethylamino)methylene)-N-methylmethanaminium hexafluorophosphate(V) (356 mg, 1.3 mmol) and 1-methyl-1H-imidazole (521 mg, 6.4 mmol) in ACN (5 mL) was stirred for 2 hours at room temperature. Upon completion, the resulting mixture was concentrated under reduced pressure. The crude residue was purified by reversed-phase flash chromatography (conditions: column: C18 silica gel; mobile phase A: water (10 mmol/L NH₄HCO₃), Mobile Phase B: MeCN; Gradient (B %): 5% to 95% in 25 min; detector: UV 254 nm) to afford tert-butyl (S)-3-fluoro-4′-(2-hydroxy-3-methylbutanamido)-[1,1′-biphenyl]-4-carboxylate (145 mg, 28% yield) as a light yellow solid. LCMS (ESI) [M+H]⁺: 388

### Step 2: tert-butyl (S)-3-fluoro-4′-(2-(((4-isopropylphenyl)carbamoyl)oxy)-3-methylbutanamido)-[1,1′-biphenyl]-4-carboxylate

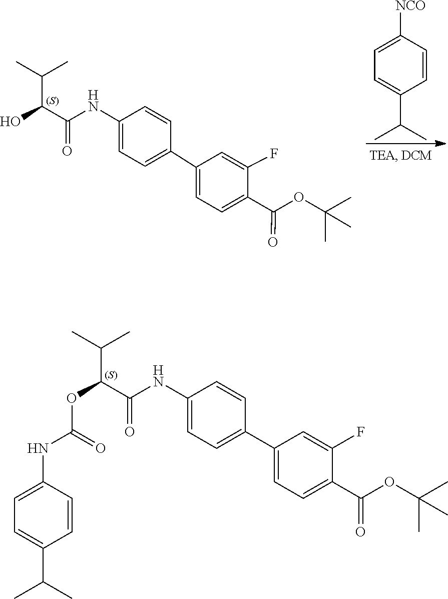

To a mixture of tert-butyl (S)-3-fluoro-4′-(2-hydroxy-3-methylbutanamido)-[1,1′-biphenyl]-4-carboxylate (135 mg, 0.3 mmol) in DCM (6 mL) were added TEA (88 mg, 0.9 mmol) and 1-isocyanato-4-isopropylbenzene (84 mg, 0.5 mmol) dropwise at 0° C. The resulting mixture was stirred at room temperature for 2 hours under nitrogen atmosphere. Upon completion, the reaction was quenched with ice water at 0° C. The resulting mixture was extracted with EtOAc (3×8 mL). The combined organic layers were dried over anhydrous Na₂SO₄, filtered and concentrated under reduced pressure. The crude residue was purified by silica gel column chromatography, eluted with PE/EA (3:1) to afford tert-butyl (S)-3-fluoro-4′-(2-(((4-isopropylphenyl)carbamoyl)oxy)-3-methylbutanamido)-[1,1′-biphenyl]-4-carboxylate (100 mg, 54% yield) as a white solid. LCMS (ESI) [M+H]⁺: 549

### Step 3: (S)-3-fluoro-4′-(2-(((4-isopropylphenyl)carbamoyl)oxy)-3-methylbutanamido)-[1,1′-biphenyl]-4-carboxylic acid

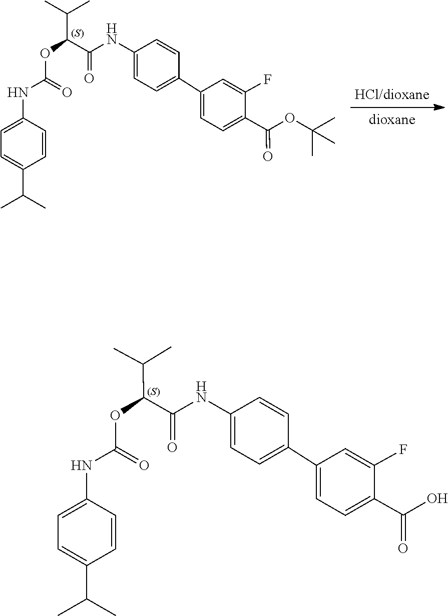

To a mixture of tert-butyl (S)-3-fluoro-4′-(2-(((4-isopropylphenyl)carbamoyl)oxy)-3-methylbutanamido)-[1,1′-biphenyl]-4-carboxylate (110 mg, 0.2 mmol) in dioxane (1 mL) was added 4M HCl in 1,4-dioxane (5 mL) dropwise at 0° C. The resulting mixture was stirred at room temperature for 2 hours. Upon completion, the resulting mixture was concentrated under reduced pressure. The crude residue was purified by Prep-HPLC (conditions: Column: XSelect CSH Fluoro Phenyl 30*150 mm, 5 μm; Mobile Phase A: Water (0.1% formic acid), Mobile Phase B: ACN; Flow rate: 60 mL/min; Gradient (B %): 41% to 62% in 15 min; Wave Length: 254 nm/220 nm; RT1 (min): 9.6) to afford (S)-3-fluoro-4′-(2-(((4-isopropylphenyl)carbamoyl)oxy)-3-methylbutanamido)-[1,1′-biphenyl]-4-carboxylic acid (36.8 mg, Compound 52; Method E, Peak 2, 1.35 min—analyzed against Compound 51) as a white solid.

| Cmpd # | Characterization Data |
|---|---|
| 52 | LCMS (ESI) [M + H]⁺: 493.2 |
| | ¹H NMR (400 MHz, DMSO-d₆) δ 13.17 (bs, 1H), 10.31 (s, 1H), 9.75 (bs, 1H), 7.92 (d, J = 8.0 Hz, |
| | 1H), 7.79-7.73 (m, 4H), 7.63-7.60 (m, 2H), 7.38 (d, J = 8.4 Hz, 2H), 7.15 (d, J = 8.4 Hz, 2H), |
| | 4.83 (d, J = 5.6 Hz, 1H), 2.84-2.80 (m, 1H), 2.33-2.17 (m, 1H), 1.20 (d, J = 24.8 Hz, 6H), 1.09 |
| | (d, J = 49.6 Hz, 6H). |

### Example 53: 3-fluoro-4′-(1-(((4-isopropylphenyl)carbamoyl)oxy)cyclopentane-1-carboxamido)-[1,1′-biphenyl]-4-carboxylic acid

### Step 1: 1-(((4-isopropylphenyl)carbamoyl)oxy)cyclopentane-1-carboxylic acid

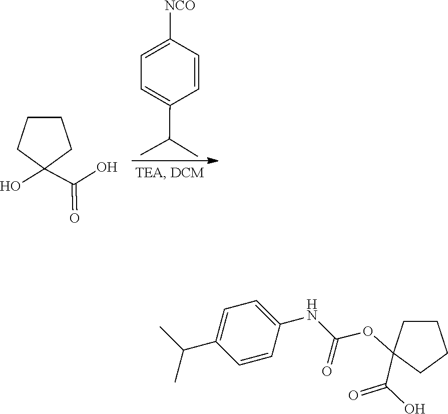

To a mixture of 1-hydroxycyclopentane-1-carboxylic acid (300 mg, 2.3 mmol) in DCM (8 mL) were added TEA (700 mg, 6.9 mmol) and 1-isocyanato-4-isopropylbenzene (743 mg, 4.6 mmol) dropwise at 0° C. The resulting mixture was stirred at room temperature for 2 hours under nitrogen atmosphere. Upon completion, the reaction was quenched with ice water at 0° C. The resulting mixture was extracted with EtOAc (3×10 mL). The combined organic layers were dried over anhydrous Na₂SO₄, filtered and concentrated under reduced pressure. The crude residue was purified by silica gel column chromatography, eluted with PE/EA (1:1) to afford 1-(((4-isopropylphenyl)carbamoyl)oxy)cyclopentane-1-carboxylic acid (180 mg, 27% yield) as a yellow solid. LCMS (ESI) [M+H]⁺: 292

### Step 2: tert-butyl 3-fluoro-4′-(1-(((4-isopropylphenyl)carbamoyl)oxy)cyclopentane-1-carboxamido)-[1,1′-biphenyl]-4-carboxylate

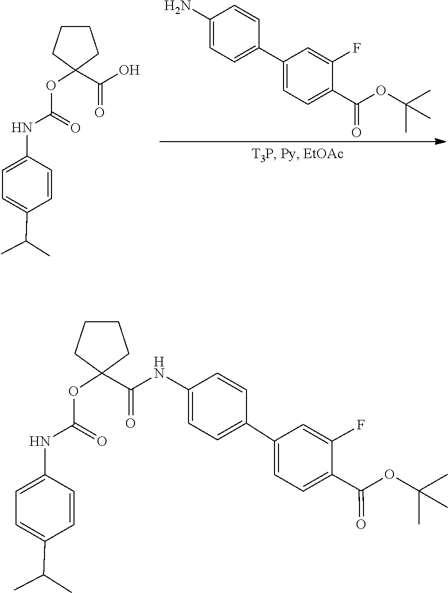 [SMILES: CC(C)c1ccc(NC(=O)OC2(C(=O)O)CCCC2)cc1 ; CC(C)(C)OC(=O)c1ccc(-c2ccc(N)cc2)cc1F ; CC(C)c1ccc(NC(=O)OC2(C(=O)Nc3ccc(-c4ccc(C(=O)OC(C)(C)C)c(F)c4)cc3)CCCC2)cc1]

To a mixture of 1-(((4-isopropylphenyl)carbamoyl)oxy)cyclopentane-1-carboxylic acid (170 mg, 0.6 mmol) and tert-butyl 4′-amino-3-fluoro-[1,1′-biphenyl]-4-carboxylate (168 mg, 0.6 mmol) in EtOAc (2 mL) was added pyridine (6 mL) and propanephosphonic acid cyclic anhydride (50% in ethyl acetate) (1.11 g, 1.7 mmol) dropwise at 0° C. The resulting mixture was warmed to room temperature and stirred for 2 hours. Upon completion, the resulting mixture was concentrated under reduced pressure. The crude residue was purified by reversed-phase flash chromatography (conditions: column: C18 silica gel; mobile phase A: water (0.1% formic acid), Mobile Phase B: MeCN; Gradient (B %): 0% to 100% in 20 min; detector, UV 254 nm. This resulted in tert-butyl 3-fluoro-4′-(1-(((4-isopropylphenyl)carbamoyl)oxy)cyclopentane-1-carboxamido)-[1,1′-biphenyl]-4-carboxylate (60 mg, 18% yield) as a yellow oil. LCMS (ESI) [M+H]⁺: 561

### Step 3: 3-fluoro-4′-(1-(((4-isopropylphenyl)carbamoyl)oxy)cyclopentane-1-carboxamido)-[1,1′-biphenyl]-4-carboxylic acid

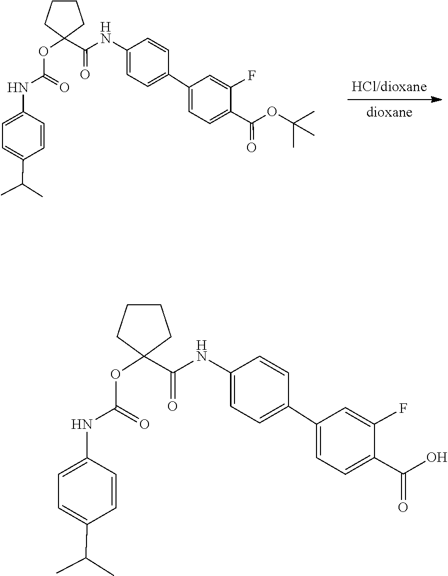

To a mixture of tert-butyl 3-fluoro-4′-(1-(((4-isopropylphenyl)carbamoyl)oxy)cyclopentane-1-carboxamido)-[1,1′-biphenyl]-4-carboxylate (58 mg, 0.1 mmol) in dioxane (0.5 mL) was added 4M HCl in 1,4-dioxane (2.5 mL) dropwise at 0° C. The resulting mixture was stirred at room temperature for 2 hours. Upon completion, the resulting mixture was concentrated under reduced pressure. The crude residue was purified by Prep-HPLC (conditions: Column: Xselect CSH Prep C18, 30*150 mm 5 μm; Mobile Phase A: Water (0.1% formic acid), Mobile Phase B: ACN; Flow rate: 60 mL/min; Gradient (B %): 54% to 72% in 10 min; Wave Length: 254 nm/220 nm; RT1 (min): 10.65) to afford 3-fluoro-4′-(1-(((4-isopropylphenyl)carbamoyl)oxy)cyclopentane-1-carboxamido)-[1,1′-biphenyl]-4-carboxylic acid (10.4 mg, 20% yield) as a white solid.

| Cmpd # | Characterization Data |
|---|---|
| 53 | LCMS (ESI) [M + H]⁺: 505.3 |
| | ¹H NMR (400 MHz, DMSO-d₆) δ 13.15 (bs, 1H), 9.74 (s, 1H), 9.59 (bs, 1H), 7.90 (d, J = 8.0 Hz, |
| | 1H), 7.87-7.79 (m, 4H), 7.75 (d, J = 9.2 Hz, 2H), 7.36 (d, J = 8.2 Hz, 2H), 7.12 (d, J = 8.4 Hz, |
| | 2H), 2.84-2.77 (m, 1H), 2.33-2.27 (m, 2H), 2.09-2.05 (m, 2H), 1.80-1.74 (m, 4H), 1.15 (d, J = |
| | 6.8 Hz, 6H). |

### Example 54A: A Synthetic Route to (S)-4-(5-fluoro-6-(1-((4-isopropylphenyl)carbamoyl)pyrrolidin-2-yl)pyridin-3-yl)benzoic acid and (R)-4-(5-fluoro-6-(1-((4-isopropylphenyl)carbamoyl)pyrrolidin-2-yl)pyridin-3-yl)benzoic acid

### Step 1: 4-(5-fluoro-6-(1-((4-isopropylphenyl)carbamoyl)pyrrolidin-2-yl)pyridin-3-yl)benzoic acid

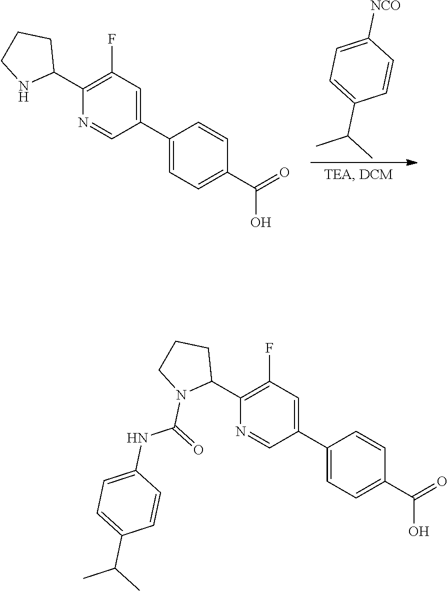

To a mixture of 4-(5-fluoro-6-(pyrrolidin-2-yl)pyridin-3-yl)benzoic acid (125 mg, 0.4 mmol) in DCM (5 mL) were added TEA (88 mg, 0.9 mmol) and 1-isocyanato-4-isopropylbenzene (84 mg, 0.5 mmol) dropwise at 0° C. under nitrogen atmosphere. The resulting mixture was stirred at room temperature for 2 hours. Upon completion, the reaction was quenched with ice water at 0° C. The resulting mixture was extracted with EtOAc (3×7 mL). The combined organic layers were dried over anhydrous Na₂SO₄, filtered and concentrated under reduced pressure. The crude residue was purified by reversed-phase flash chromatography (conditions: column: C18 silica gel; mobile phase A: water (0.1% formic acid), Mobile Phase B: MeCN; Gradient (B %): 0% to 100% in 20 min; detector: UV 254 nm) to afford 4-(5-fluoro-6-(1-((4-isopropylphenyl)carbamoyl)pyrrolidin-2-yl)pyridin-3-yl)benzoic acid (38 mg, 19% yield) as a yellow solid. LCMS [M+H]⁺: 448

### Step 2: (S)-4-(5-fluoro-6-(1-((4-isopropylphenyl)carbamoyl)pyrrolidin-2-yl)pyridin-3-yl)benzoic acid and (R)-4-(5-fluoro-6-(1-((4-isopropylphenyl)carbamoyl)pyrrolidin-2-yl)pyridin-3-yl)benzoic acid

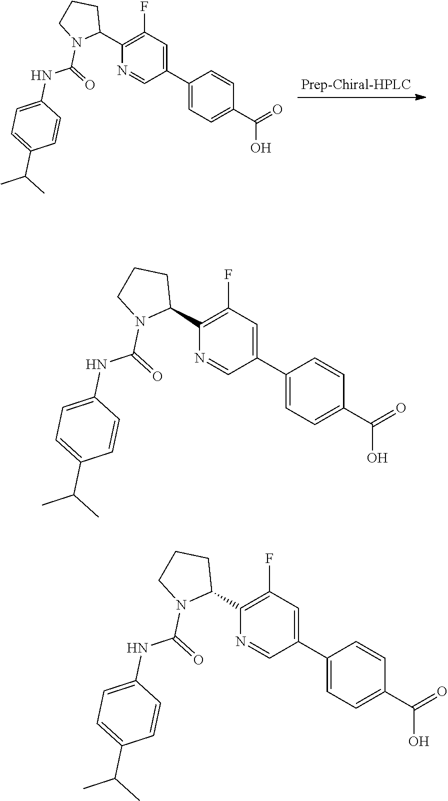 [SMILES: CC(C)c1ccc(NC(=O)N2CCCC2c2ncc(-c3ccc(C(=O)O)cc3)cc2F)cc1 ; CC(C)c1ccc(NC(=O)N2CCC[C@H]2c2ncc(-c3ccc(C(=O)O)cc3)cc2F)cc1 ; CC(C)c1ccc(NC(=O)N2CCC[C@@H]2c2ncc(-c3ccc(C(=O)O)cc3)cc2F)cc1]

4-(5-fluoro-6-(1-((4-isopropylphenyl)carbamoyl)pyrrolidin-2-yl)pyridin-3-yl)benzoic acid (99 mg) was separated by Prep-Chiral HPLC (conditions: Column: CHIRAL ART Cellulose-SC, 3*25 cm, 5 μm; Mobile Phase A: MTBE (0.1% FA), Mobile Phase B: MeOH; Flow rate: 40 mL/min; Gradient (B %): isocratic 10% B; Wave Length: 274/202 nm; RT1 (min): 7.21; RT2 (min): 10.22) to afford first peak (Compound 54a; Method U, Peak 1, 0.82 min) (S)-4-(5-fluoro-6-(1-((4-isopropylphenyl)carbamoyl)pyrrolidin-2-yl)pyridin-3-yl)benzoic acid (36.5 mg) as a white solid and second (Compound 54b; Method U, Peak 2, 1.20 min) (R)-4-(5-fluoro-6-(1-((4-isopropylphenyl)carbamoyl)pyrrolidin-2-yl)pyridin-3-yl)benzoic acid (35.1 mg) as an off-white solid.

| Cmpd # | Characterization Data |
|---|---|
| 54a | LCMS (ESI) [M + H]⁺: 448.2 |
| | ¹H NMR (400 MHz, DMSO-d₆) δ 13.02 (bs, 1H), 8.73 (s, 1H), 8.14 (s, 1H), 8.08-8.01 (m, 3H), |
| | 7.89 (d, J = 8.0 Hz, 2H), 7.32 (d, J = 8.4 Hz, 2H), 7.04 (d, J = 8.4 Hz, 2H), 5.35-5.32 (m, 1H), |
| | 3.77-3.73 (m, 1H), 3.63-3.60 (m, 1H), 2.83-2.76 (m, 1H), 2.31-2.28 (m, 1H), 2.19-2.08 (m, |
| | 1H), 2.07-1.94 (m, 1H), 1.92-1.84 (m, 1H), 1.14 (d, J = 6.8 Hz, 6H). |
| 54b | LCMS (ESI) [M + H]⁺: 448.2 |
| | ¹H NMR (400 MHz, DMSO-d₆) δ 13.01 (bs, 1H), 8.73 (s, 1H), 8.17-8.13 (m, 1H), 8.08-8.01 |
| | (m, 3H), 7.88 (d, J = 8.4 Hz, 2H), 7.32 (d, J = 8.4 Hz, 2H), 7.04 (d, J = 8.8 Hz, 2H), 5.35-5.32 |
| | (m, 1H), 3.79-3.73 (m, 1H), 3.65-3.61 (m, 1H), 2.79-2.76 (m, 1H), 2.49-2.35 (m, 1H), 2.21- |
| | 2.11 (m, 1H), 2.09-1.95 (m, 1H), 1.92-1.85 (m, 1H), 1.14 (d, J = 6.8 Hz, 6H). |

### Example 54B-1: An Alternative Synthetic Route for (S)-4-(5-fluoro-6-(1-((4-isopropylphenyl) carbamoyl)pyrrolidin-2-yl)pyridin-3-yl)benzoic acid

### Step 1: tert-butyl (2S,4R)-2-(3-fluoro-5-(4-(methoxycarbonyl)phenyl)pyridin-2-yl)-4-hydroxypyrrolidine-1-carboxylate

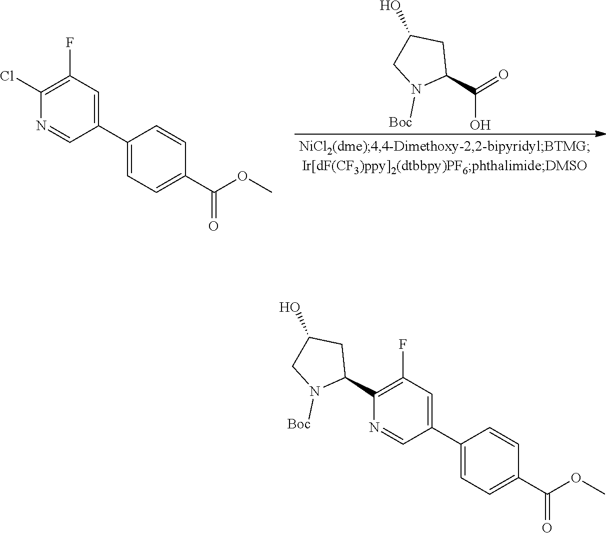 [SMILES: COC(=O)c1ccc(-c2cnc(Cl)c(F)c2)cc1 ; CC(C)(C)OC(=O)N1C[C@H](O)C[C@H]1C(=O)O ; COC(=O)c1ccc(-c2cnc([C@@H]3C[C@@H](O)CN3C(=O)OC(C)(C)C)c(F)c2)cc1]

A mixture of methyl 4-(6-chloro-5-fluoropyridin-3-yl)benzoate (500 mg, 1.88 mmol), (2S,4R)-1-(tert-butoxycarbonyl)-4-hydroxypyrrolidine-2-carboxylic acid (653 mg, 2.82 mmol), dichloronickel·1,2-dimethoxyethane (41.4 mg, 0.19 mmol), 4,4′-dimethoxy-2,2′-bipyridine (48.8 mg, 0.23 mmol), N″-tert-butyl-N,N,N′,N′-tetramethylguanidine (484 mg, 2.82 mmol), Ir[dF(CF₃)ppy]₂(dtbpy))PF₆ (42.2 mg, 0.038 mmol) and 2,3-dihydro-1H-isoindole-1,3-dione (277 mg, 1.88 mmol) in DMSO (8 mL) was stirred at 25° C. under nitrogen atmosphere. The resulting mixture was stirred and irradiated with 450 nm blue LEDs for 24 hours. Upon completion, the mixture was directly purified by reversed-phase flash chromatography (conditions: column, C18 silica gel; mobile phase A: H₂O, Mobile Phase B: MeCN (0.1% FA); Gradient (B %): 10% to 50% in 10 min; detector, UV 254 nm) to afford tert-butyl (2S,4R)-2-(3-fluoro-5-(4-(methoxycarbonyl)phenyl)pyridin-2-yl)-4-hydroxypyrrolidine-1-carboxylate (450 mg, 57% yield) as a brown oil. LCMS (ESI) [M+H]⁺: 417.1

### Step 2: tert-butyl (2S,4R)-2-(3-fluoro-5-(4-(methoxycarbonyl)phenyl)pyridin-2-yl)-4-((phenoxycarbonothioyl)oxy)pyrrolidine-1-carboxylate

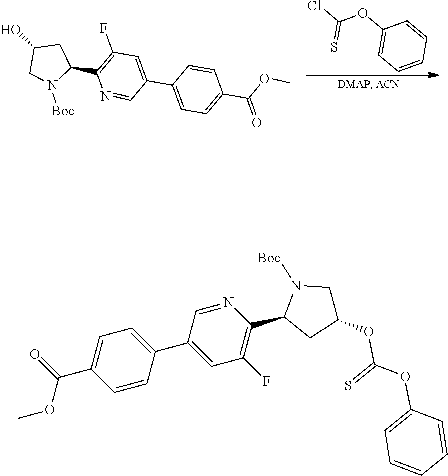

To a stirred solution of tert-butyl (2S,4R)-2-(3-fluoro-5-(4-(methoxycarbonyl)phenyl)pyridin-2-yl)-4-hydroxypyrrolidine-1-carboxylate (400 mg, 0.96 mmol) and DMAP (235 mg, 1.92 mmol) in MeCN (4 mL) was added phenyl chloromethanethioate (199 mg, 1.15 mmol) in portions at 0° C. The resulting mixture was warmed to room temperature and stirred for 12 hours. Upon completion, the mixture was diluted with water (10 mL) and extracted with EtOAc (3×10 mL). The combined organic layers were washed with H₂O (10 mL), dried over anhydrous Na₂SO₄, filtered and concentrated under reduced pressure to give crude tert-butyl (2S,4R)-2-(3-fluoro-5-(4-(methoxycarbonyl)phenyl)pyridin-2-yl)-4-((phenoxycarbonothioyl)oxy)pyrrolidine-1-carboxylate (380 mg, 71% yield) as a yellow solid, which was used in the next step directly without further purification. LCMS (ESI) [M+H]⁺: 553.1

### Step 3: tert-butyl (S)-2-(3-fluoro-5-(4-(methoxycarbonyl)phenyl)pyridin-2-yl)pyrrolidine-1-carboxylate

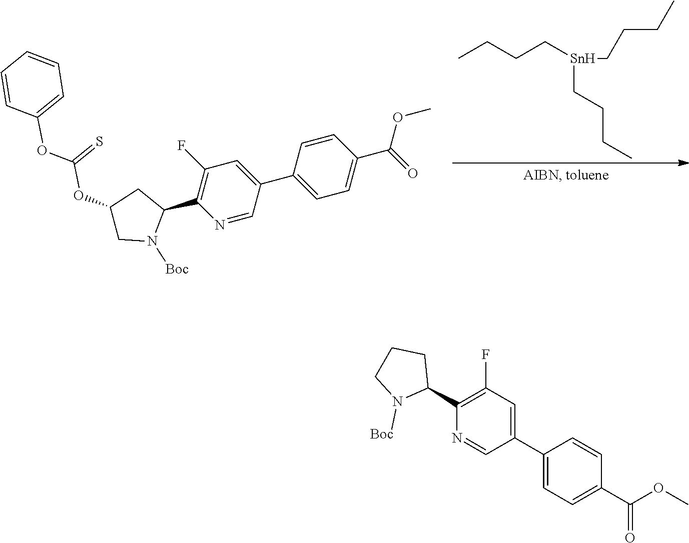

A solution of tert-butyl (2S,4R)-2-(3-fluoro-5-(4-(methoxycarbonyl)phenyl)pyridin-2-yl)-4-((phenoxycarbonothioyl)oxy)pyrrolidine-1-carboxylate (380 mg, 0.69 mmol) in toluene (3 mL) was treated with AIBN (226 mg, 1.38 mmol) at 25° C. followed by the dropwise addition of tributyltin hydride (300 mg, 1.03 mmol). The resulting mixture was stirred at 90° C. for 3 hours. Upon completion, the resulting mixture was concentrated under reduced pressure. The crude residue was purified by silica gel column chromatography, eluted with PE/EA (1/1) to afford tert-butyl (S)-2-(3-fluoro-5-(4-(methoxycarbonyl)phenyl)pyridin-2-yl)pyrrolidine-1-carboxylate (200 mg, 73% yield) as a yellow oil. LCMS (ESI) [M+H]⁺: 401.1

### Step 4: methyl (S)-4-(5-fluoro-6-(pyrrolidin-2-yl)pyridin-3-yl)benzoate

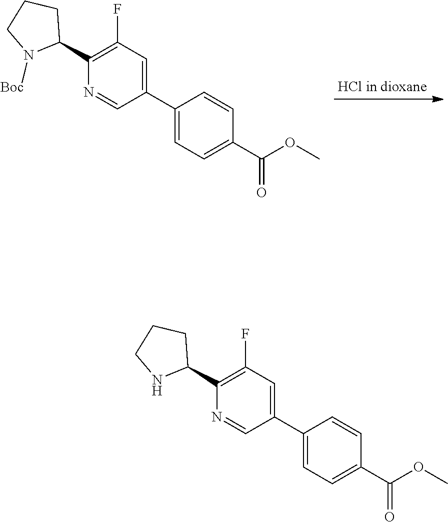 [SMILES: COC(=O)c1ccc(-c2cnc([C@@H]3CCCN3C(=O)OC(C)(C)C)c(F)c2)cc1 ; COC(=O)c1ccc(-c2cnc([C@@H]3CCCN3)c(F)c2)cc1]

To a solution of tert-butyl (S)-2-(3-fluoro-5-(4-(methoxycarbonyl)phenyl)pyridin-2-yl)pyrrolidine-1-carboxylate (200 mg, 0.50 mmol) in dioxane (2 mL) was added HCl (2 mL, 8 mmol, 4 M in dioxane) at room temperature. After 1 hour, the resulting mixture was concentrated under reduced pressure. The crude residue was purified by reversed-phase flash chromatography (conditions: column, C18 silica gel; mobile phase A: water, Mobile phase B: MeCN (0.1% FA); Gradient (B %): 10% to 50% in 20 min; detector: UV 254 nm) to afford methyl (S)-4-(5-fluoro-6-(pyrrolidin-2-yl)pyridin-3-yl)benzoate (120 mg, 80% yield) as a yellow oil. LCMS (ESI) [M+H]⁺: 301.1

### Step 5: methyl (S)-4-(5-fluoro-6-(1-((4-isopropylphenyl)carbamoyl)pyrrolidin-2-yl)pyridin-3-yl)benzoate

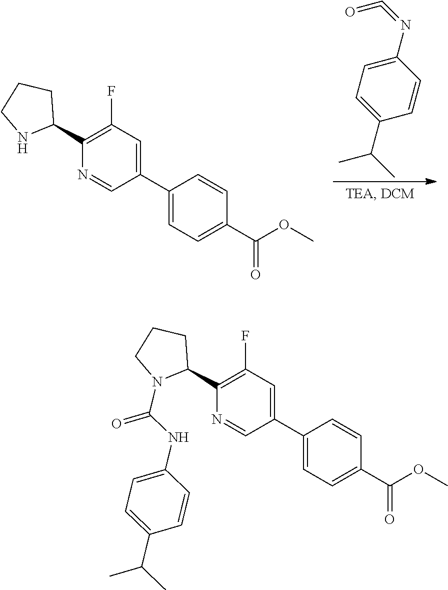

A solution of methyl (S)-4-(5-fluoro-6-(pyrrolidin-2-yl)pyridin-3-yl)benzoate (120 mg, 0.400 mmol), 1-isocyanato-4-isopropylbenzene (77 mg, 0.48 mmol) and triethylamine (80.9 mg, 0.80 mmol) in DCM (1 mL) was stirred at 25° C. for 1 hour. Upon completion, the resulting mixture was concentrated under reduced pressure. The crude residue was purified by silica gel column chromatography, eluted with PE/EA (3/1) to afford methyl (S)-4-(5-fluoro-6-(1-((4-isopropylphenyl)carbamoyl)pyrrolidin-2-yl)pyridin-3-yl)benzoate (100 mg, 54% yield) as a yellow oil. LCMS (ESI) [M+H]⁺: 462.2

### Step 6: (S)-4-(5-fluoro-6-(1-((4-isopropylphenyl)carbamoyl)pyrrolidin-2-yl)pyridin-3-yl)benzoic acid

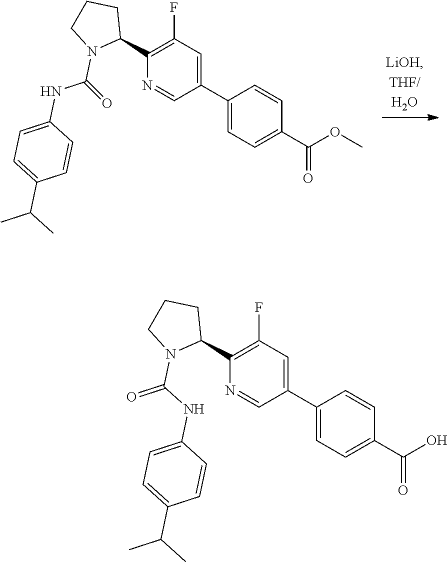

A solution of methyl (S)-4-(5-fluoro-6-(1-((4-isopropylphenyl)carbamoyl)pyrrolidin-2-yl)pyridin-3-yl)benzoate (100 mg, 0.217 mmol) and LiOH monohydrate (18.0 mg, 0.43 mmol) in THF (1 mL) and H₂O (1 mL) was stirred at 40° C. for 2 hours. Upon completion, the mixture was acidified to pH=2 with HCl (1 M in water). The resulting mixture was concentrated under reduced pressure. The crude residue was purified by reversed-phase flash chromatography (conditions: column: C18 silica gel; mobile phase A: H₂O, Mobile Phase B: MeCN (0.1% FA); Gradient (B %): 10% to 60% in 20 min; detector: UV 254 nm) to afford (S)-4-(5-fluoro-6-(1-((4-isopropylphenyl)carbamoyl)pyrrolidin-2-yl)pyridin-3-yl)benzoic acid (59.5 mg, 61% yield; Method U, Peak 1, 0.84 min) as a white solid.

| Cmpd # | Characterization Data |
|---|---|
| 54a | LCMS (ESI) [M + H]⁺: 448.2 |
| | ¹H NMR (400 MHz, DMSO-d₆) δ 13.02 (bs, 1H), 8.73 (s, 1H), 8.14 (s, 1H), 8.08-8.01 (m, 3H), |
| | 7.89 (d, J = 8.0 Hz, 2H), 7.32 (d, J = 8.4 Hz, 2H), 7.04 (d, J = 8.4 Hz, 2H), 5.35-5.32 (m, 1H), |
| | 3.77-3.73 (m, 1H), 3.63-3.60 (m, 1H), 2.83-2.76 (m, 1H), 2.31-2.28 (m, 1H), 2.19-2.08 (m, |
| | 1H), 2.07-1.94 (m, 1H), 1.92-1.84 (m, 1H), 1.14 (d, J = 6.8 Hz, 6H). |

### Example 54B-2: An Alternative Synthetic Route for (R)-4-(5-fluoro-6-(1-((4-isopropylphenyl) carbamoyl)pyrrolidin-2-yl)pyridin-3-yl)benzoic acid

### Step 1: tert-butyl (2R,4S)-2-(3-fluoro-5-(4-(methoxycarbonyl)phenyl)pyridin-2-yl)-4-hydroxypyrrolidine-1-carboxylate

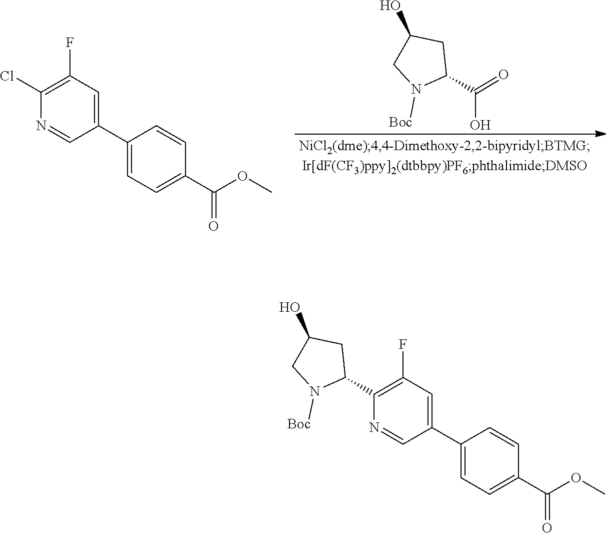 [SMILES: COC(=O)c1ccc(-c2cnc(Cl)c(F)c2)cc1 ; CC(C)(C)OC(=O)N1C[C@@H](O)C[C@@H]1C(=O)O ; COC(=O)c1ccc(-c2cnc([C@H]3C[C@H](O)CN3C(=O)OC(C)(C)C)c(F)c2)cc1]

A mixture of methyl 4-(6-chloro-5-fluoropyridin-3-yl)benzoate (500 mg, 1.88 mmol), (2R,4S)-1-(tert-butoxycarbonyl)-4-hydroxypyrrolidine-2-carboxylic acid (653 mg, 2.82 mmol), NiCl₂(DME) (41.4 mg, 0.19 mmol), 4,4′-dimethoxy-2,2′-bipyridine (48.8 mg, 0.23 mmol), N″-tert-butyl-N,N,N′,N′-tetramethylguanidine (484 mg, 2.82 mmol), Ir[dF(CF3)ppy]2(dtbpy))PF6 (42.2 mg, 0.04 mmol) and 2,3-dihydro-1H-isoindole-1,3-dione (277 mg, 1.88 mmol) in DMSO (8 mL) was stirred at 25° C. under nitrogen atmosphere and irradiated with 450 nm blue LEDs for 12 hours. Upon completion, the mixture was directly purified by reversed-phase flash chromatography (conditions: column, C18 silica gel; mobile phase A: water, Mobile phase B: MeCN (0.1% FA); Gradient (B %): 10% to 50% in 10 min; detector, UV 254 nm) to afford tert-butyl (2R,4S)-2-(3-fluoro-5-(4-(methoxycarbonyl)phenyl)pyridin-2-yl)-4-hydroxypyrrolidine-1-carboxylate (430 mg, 55% yield) as a brown oil. LCMS (ESI) [M+H]⁺: 417.1

### Step 2: tert-butyl (2R,4S)-2-(3-fluoro-5-(4-(methoxycarbonyl)phenyl)pyridin-2-yl)-4-((phenoxycarbonothioyl)oxy)pyrrolidine-1-carboxylate

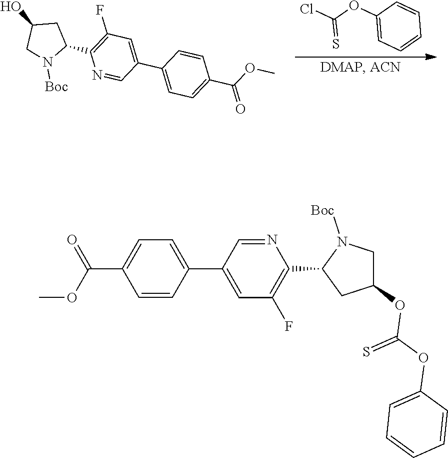 [SMILES: COC(=O)c1ccc(-c2cnc([C@H]3C[C@H](O)CN3C(=O)OC(C)(C)C)c(F)c2)cc1 ; S=C(Cl)Oc1ccccc1 ; COC(=O)c1ccc(-c2cnc([C@H]3C[C@H](OC(=S)Oc4ccccc4)CN3C(=O)OC(C)(C)C)c(F)c2)cc1]

To a stirred solution of tert-butyl (2R,4S)-2-(3-fluoro-5-(4-(methoxycarbonyl)phenyl)pyridin-2-yl)-4-hydroxypyrrolidine-1-carboxylate (400 mg, 0.96 mmol) and DMAP (235 mg, 1.92 mmol) in MeCN (4 mL) was added phenyl chloromethanethioate (199 mg, 1.15 mmol) in portions at 0° C. The reaction mixture was warmed to room temperature and stirred for 12 hours. Upon completion, the mixture was diluted with water (10 mL) and extracted with EtOAc (3×10 mL). The combined organic layers were washed with H₂O (10 mL), dried over anhydrous Na₂SO₄, filtered and concentrated under reduced pressure to give crude tert-butyl (2R,4S)-2-(3-fluoro-5-(4-(methoxycarbonyl)phenyl)pyridin-2-yl)-4-((phenoxycarbonothioyl)oxy)pyrrolidine-1-carboxylate (350 mg, 65% yield) as a yellow solid, which was used in the next step directly without further purification. LCMS (ESI) [M+H]⁺: 553.1

### Step 3: tert-butyl (R)-2-(3-fluoro-5-(4-(methoxycarbonyl)phenyl)pyridin-2-yl)pyrrolidine-1-carboxylate

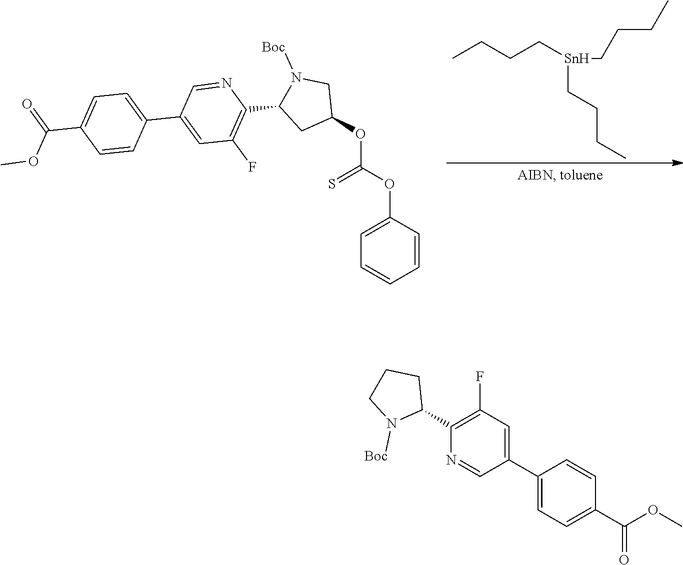

A solution of tert-butyl (2R,4S)-2-(3-fluoro-5-(4-(methoxycarbonyl)phenyl)pyridin-2-yl)-4-((phenoxycarbonothioyl)oxy)pyrrolidine-1-carboxylate (350 mg, 0.63 mmol) in toluene (3 mL) was treated with AIBN (208 mg, 1.27 mmol) at 25° C. followed by the dropwise addition of tributyltin hydride (277 mg, 0.95 mmol). The resulting mixture was stirred at 90° C. for 3 hours. Upon completion, the resulting mixture was concentrated under reduced pressure. The crude residue was purified by silica gel column chromatography, eluted with PE/EA (1/1) to afford tert-butyl (R)-2-(3-fluoro-5-(4-(methoxycarbonyl)phenyl)pyridin-2-yl)pyrrolidine-1-carboxylate (200 mg, 79% yield) as a yellow oil. LCMS (ESI) [M+H]⁺: 401.1

### Step 4: methyl (R)-4-(5-fluoro-6-(pyrrolidin-2-yl)pyridin-3-yl)benzoate

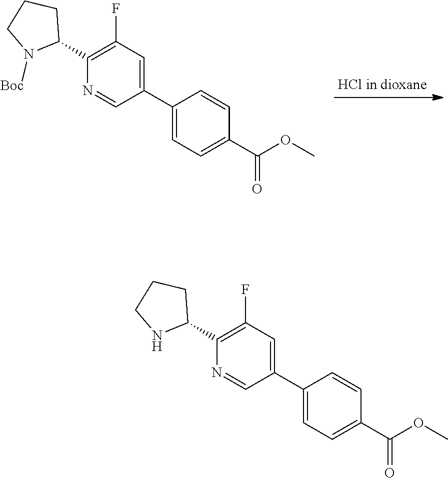 [SMILES: COC(=O)c1ccc(-c2cnc([C@H]3CCCN3C(=O)OC(C)(C)C)c(F)c2)cc1 ; COC(=O)c1ccc(-c2cnc([C@H]3CCCN3)c(F)c2)cc1]

To a solution of tert-butyl (R)-2-(3-fluoro-5-(4-(methoxycarbonyl)phenyl)pyridin-2-yl)pyrrolidine-1-carboxylate (200 mg, 0.50 mmol) in dioxane (2 mL) was added HCl (2 mL, 8 mmol, 4 M in dioxane) and the mixture was stirred at 25° C. for 1 hour. Upon completion, the resulting mixture was concentrated under reduced pressure. The crude residue was purified by reversed-phase flash chromatography (conditions: column: C18 silica gel; mobile phase A: water, Mobile Phase B: MeCN (0.1% FA); Gradient (B %): 10% to 60% in 20 min; detector: UV 254 nm) to afford methyl (R)-4-(5-fluoro-6-(pyrrolidin-2-yl)pyridin-3-yl)benzoate (140 mg, 93% yield) as a yellow oil. LCMS (ESI) [M+H]⁺: 301.1

### Step 5: methyl (R)-4-(5-fluoro-6-(1-((4-isopropylphenyl)carbamoyl)pyrrolidin-2-yl)pyridin-3-yl)benzoate

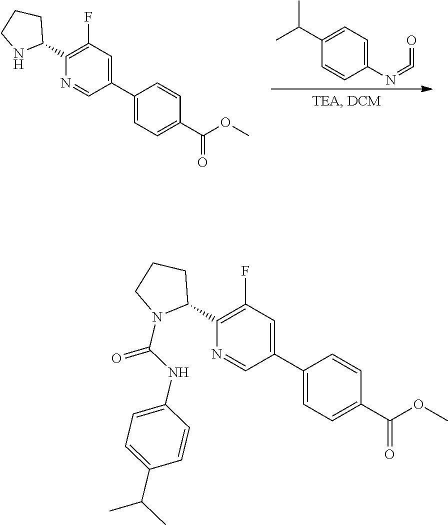 [SMILES: COC(=O)c1ccc(-c2cnc([C@H]3CCCN3)c(F)c2)cc1 ; CC(C)c1ccc(N=C=O)cc1 ; COC(=O)c1ccc(-c2cnc([C@H]3CCCN3C(=O)Nc3ccc(C(C)C)cc3)c(F)c2)cc1]

A solution of methyl (R)-4-(5-fluoro-6-(pyrrolidin-2-yl)pyridin-3-yl)benzoate (140 mg, 0.47 mmol), 1-isocyanato-4-isopropylbenzene (77 mg, 0.48 mmol) and triethylamine (80.9 mg, 0.80 mmol) in DCM (2 mL) was stirred at 25° C. for 1 hour. Upon completion, the resulting mixture was concentrated under reduced pressure. The crude residue was purified by silica gel column chromatography, eluted with PE/EA (3/1) to afford methyl (R)-4-(5-fluoro-6-(1-((4-isopropylphenyl)carbamoyl)pyrrolidin-2-yl)pyridin-3-yl)benzoate (130 mg, 60% yield) as a yellow oil. LCMS (ESI) [M+H]⁺: 462.2

### Step 6: (R)-4-(5-fluoro-6-(1-((4-isopropylphenyl)carbamoyl)pyrrolidin-2-yl)pyridin-3-yl)benzoic acid

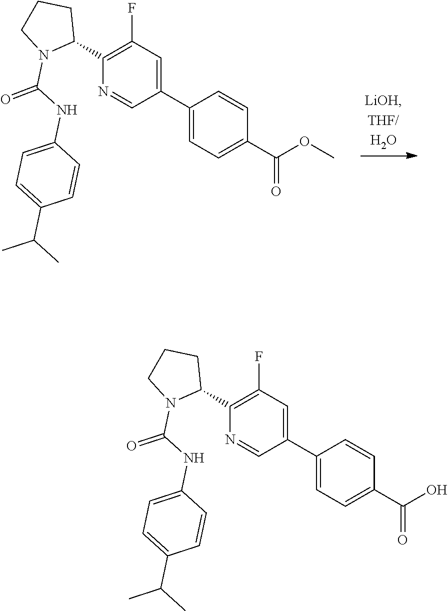

A solution of methyl (R)-4-(5-fluoro-6-(1-((4-isopropylphenyl)carbamoyl)pyrrolidin-2-yl)pyridin-3-yl)benzoate (130 mg, 0.28 mmol) and LiOH monohydrate (23.5 mg, 0.56 mmol) in THF (1 mL) and H₂O (1 mL) was stirred at 40° C. for 2 hours. Upon completion, the mixture was acidified to pH=2 with HCl (1 M in water). The resulting mixture was concentrated under reduced pressure. The crude residue was purified by reversed-phase flash chromatography (conditions: column: C18 silica gel; mobile phase A: water, Mobile Phase B: MeCN (0.1% FA); Gradient (B %): 10% to 70% in 20 min; detector: UV 254 nm) to afford (R)-4-(5-fluoro-6-(1-((4-isopropylphenyl)carbamoyl)pyrrolidin-2-yl)pyridin-3-yl)benzoic acid (95.2 mg, 76% yield; Method U, Peak 2, 1.23 min) as a white solid.

| Cmpd # | Characterization Data |
|---|---|
| 54b | LCMS (ESI) [M + H]⁺: 448.2 |
| | ¹H NMR (400 MHz, DMSO-d₆) δ 13.01 (bs, 1H), 8.73 (s, 1H), 8.17-8.13 (m, 1H), 8.08-8.01 |
| | (m, 3H), 7.88 (d, J = 8.4 Hz, 2H), 7.32 (d, J = 8.4 Hz, 2H), 7.04 (d, J = 8.8 Hz, 2H), 5.35-5.32 |
| | (m, 1H), 3.79-3.73 (m, 1H), 3.65-3.61 (m, 1H), 2.79-2.76 (m, 1H), 2.49-2.35 (m, 1H), 2.21- |
| | 2.11 (m, 1H), 2.09-1.95 (m, 1H), 1.92-1.85 (m, 1H), 1.14 (d, J = 6.8 Hz, 6H). |

### Example 57: (S)-4-(5-fluoro-6-(1-((4-(trifluoromethyl)phenyl)carbamoyl)pyrrolidin-2-yl)pyridin-3-yl)benzoic acid and (R)-4-(5-fluoro-6-(1-((4-(trifluoromethyl)phenyl)carbamoyl)pyrrolidin-2-yl)pyridin-3-yl)benzoic acid

### Step 1: 4-(5-fluoro-6-(1-((4-(trifluoromethyl)phenyl)carbamoyl)pyrrolidin-2-yl)pyridin-3-yl)benzoic acid

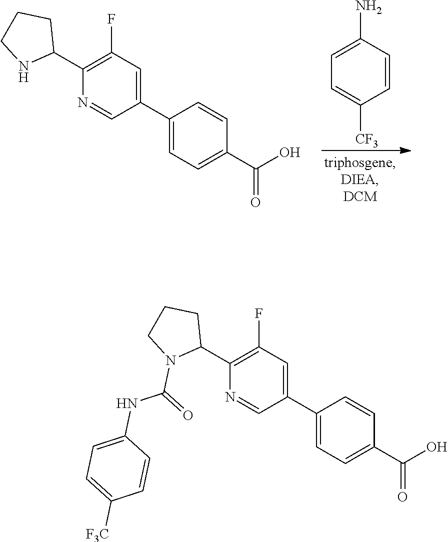 [SMILES: O=C(O)c1ccc(-c2cnc(C3CCCN3)c(F)c2)cc1 ; Nc1ccc(C(F)(F)F)cc1 ; O=C(O)c1ccc(-c2cnc(C3CCCN3C(=O)Nc3ccc(C(F)(F)F)cc3)c(F)c2)cc1]

To a mixture of 4-(trifluoromethyl)aniline (62 mg, 0.4 mmol) in DCM (5 mL) were added DIEA (248 mg, 1.9 mmol) and ditrichloromethyl carbonate (57 mg, 0.2 mmol) dropwise at 0° C. under nitrogen atmosphere. After 30 minutes, the reaction was warmed to room temperature and 4-(5-fluoro-6-(pyrrolidin-2-yl)pyridin-3-yl)benzoic acid (110 mg, 0.4 mmol) was added dropwise over 3 minutes. The resulting mixture was stirred at room temperature for an additional 2 hours. Upon completion, the reaction was quenched with ice water at 0° C. The resulting mixture was extracted with EtOAc (3×7 mL). The combined organic layers were dried over anhydrous Na₂SO₄, filtered and concentrated under reduced pressure. The crude residue was purified by silica gel column chromatography, eluted with CH₂Cl₂/MeOH (5:1) to afford 4-(5-fluoro-6-(1-((4-(trifluoromethyl)phenyl)carbamoyl)pyrrolidin-2-yl)pyridin-3-yl)benzoic acid (70 mg, 38% yield) as a yellow solid. LCMS [M+H]⁺: 474

### Step 2: (S)-4-(5-fluoro-6-(1-((4-(trifluoromethyl)phenyl)carbamoyl)pyrrolidin-2-yl)pyridin-3-yl)benzoic acid and (R)-4-(5-fluoro-6-(1-((4-(trifluoromethyl)phenyl)carbamoyl)pyrrolidin-2-yl)pyridin-3-yl)benzoic acid

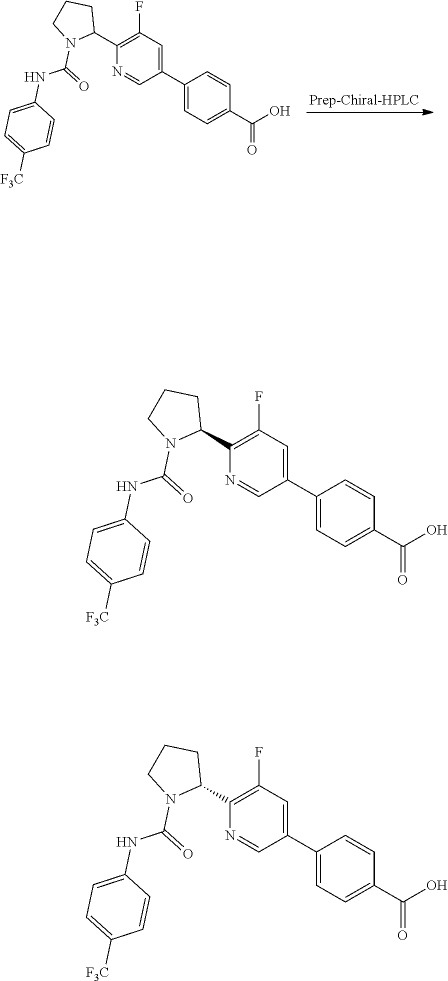 [SMILES: O=C(O)c1ccc(-c2cnc(C3CCCN3C(=O)Nc3ccc(C(F)(F)F)cc3)c(F)c2)cc1 ; O=C(O)c1ccc(-c2cnc([C@@H]3CCCN3C(=O)Nc3ccc(C(F)(F)F)cc3)c(F)c2)cc1 ; O=C(O)c1ccc(-c2cnc([C@H]3CCCN3C(=O)Nc3ccc(C(F)(F)F)cc3)c(F)c2)cc1]

4-(5-fluoro-6-(1-((4-(trifluoromethyl)phenyl)carbamoyl)pyrrolidin-2-yl)pyridin-3-yl)benzoic acid (68 mg) was separated by Prep-Chiral HPLC (conditions: Column: CHIRALPAK ID, 2*25 cm, 5 μm; Mobile Phase A: Hexane:MTBE=1:1 (0.1% FA), Mobile Phase B: MeOH; Flow rate: 20 mL/min; Gradient (B %): isocratic 10% B; Wave Length: 252/286 nm; RT1 (min): 5.53; RT2 (min): 9.88) to afford first peak (Compound 57a; Method T, Peak 1, 0.81 min) (S)-4-(5-fluoro-6-(1-((4-(trifluoromethyl)phenyl)carbamoyl)pyrrolidin-2-yl)pyridin-3-yl)benzoic acid (14.9 mg) as a white solid and second peak (Compound 57b; Method T, Peak 2, 1.20 min) (R)-4-(5-fluoro-6-(1-((4-(trifluoromethyl)phenyl)carbamoyl)pyrrolidin-2-yl)pyridin-3-yl)benzoic acid (8.9 mg) as a white solid.

| Cmpd # | Characterization Data |
|---|---|
| 57a | LCMS (ESI) [M + H]⁺: 474.1 |
| | ¹H NMR (400 MHz, DMSO-d₆) δ 8.73-8.67 (m, 2H), 8.09-8.02 (m, 3H), 7.89 (d, J = 8.2 Hz, |
| | 2H), 7.68 (d, J = 8.6 Hz, 2H), 7.53 (d, J = 8.6 Hz, 2H), 5.41-5.35 (m, 1H), 3.83-3.78 (m, 1H), |
| | 3.70-3.64 (m, 1H), 2.41-2.33 (m, 1H), 2.12-1.99 (m, 2H), 1.95-1.87 (m, 1H). |
| 57b | LCMS (ESI) [M + H]⁺: 474.1 |
| | ¹H NMR (400 MHz, DMSO-d₆) δ 8.73-8.67 (m, 2H), 8.09-8.02 (m, 3H), 7.89 (d, J = 8.2 Hz, |
| | 2H), 7.68 (d, J = 8.6 Hz, 2H), 7.53 (d, J = 8.6 Hz, 2H), 5.42-5.32 (m, 1H), 3.83-3.78 (m, 1H), |
| | 3.70-3.64 (m, 1H), 2.41-2.33 (m, 1H), 2.12-1.99 (m, 2H), 1.95-1.87 (m, 1H). |

### Example 58: (S)-4-(6-(1-((4-cyclopropyl-3-(trifluoromethyl)phenyl)carbamoyl)pyrrolidin-2-yl)-5-fluoropyridin-3-yl)benzoic acid and (R)-4-(6-(1-((4-cyclopropyl-3-(trifluoromethyl)phenyl)carbamoyl)pyrrolidin-2-yl)-5-fluoropyridin-3-yl)benzoic acid

### Step 1: 4-(6-(1-((4-cyclopropyl-3-(trifluoromethyl)phenyl)carbamoyl)pyrrolidin-2-yl)-5-fluoropyridin-3-yl)benzoic acid

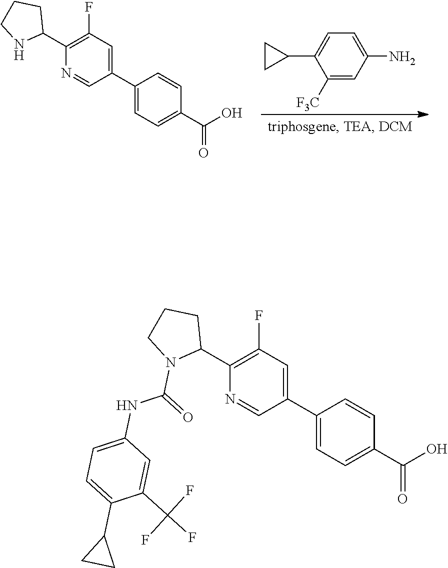 [SMILES: O=C(O)c1ccc(-c2cnc(C3CCCN3)c(F)c2)cc1 ; Nc1ccc(C2CC2)c(C(F)(F)F)c1 ; O=C(O)c1ccc(-c2cnc(C3CCCN3C(=O)Nc3ccc(C4CC4)c(C(F)(F)F)c3)c(F)c2)cc1]

To a solution of triphosgene (37 mg, 0.1 mmol) in DCM (0.5 mL) was added 4-cyclopropyl-3-(trifluoromethyl)aniline (75 mg, 0.3 mmol) and triethylamine (127 mg, 1.2 mmol) in DCM (0.5 mL) dropwise at 0° C. After 10 minutes, the reaction was warmed to room temperature and 4-(5-fluoro-6-(pyrrolidin-2-yl)pyridin-3-yl)benzoic acid (90 mg, 0.2 mmol) was added in portions. The resulting mixture was stirred at room temperature for an additional 1 hour. Upon completion, the resulting mixture was concentrated under reduced pressure. The crude residue was purified by reversed-phase flash chromatography (conditions: column: C18 silica gel; mobile phase A: water (0.1% formic acid), Mobile Phase B: MeCN; Gradient (B %): 5% to 95% in 15 min; detector, UV 254 nm) to afford 4-(6-(1-((4-cyclopropyl-3-(trifluoromethyl)phenyl)carbamoyl) pyrrolidin-2-yl)-5-fluoropyridin-3-yl)benzoic acid (126 mg, 78% yield) as a white solid. LCMS (ESI) [M+H]⁺: 514

### Step 2: (S)-4-(6-(1-((4-cyclopropyl-3-(trifluoromethyl)phenyl)carbamoyl)pyrrolidin-2-yl)-5-fluoropyridin-3-yl)benzoic acid and (R)-4-(6-(1-((4-cyclopropyl-3-(trifluoromethyl)phenyl)carbamoyl)pyrrolidin-2-yl)-5-fluoropyridin-3-yl)benzoic acid

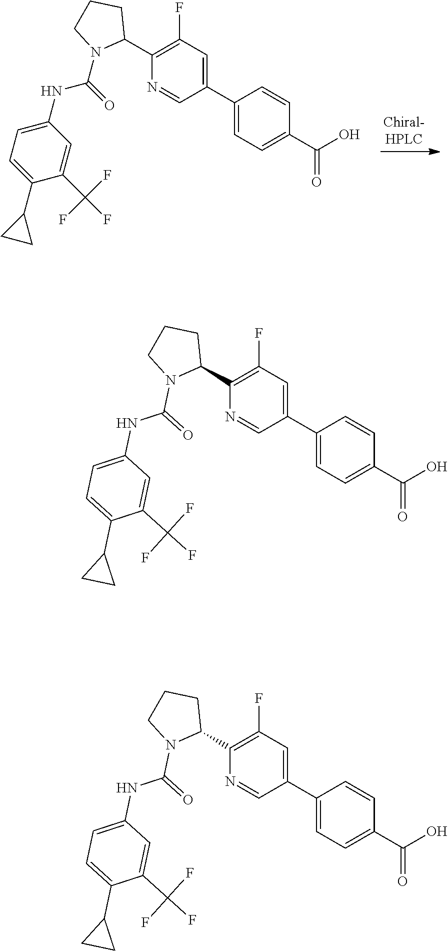 [SMILES: O=C(O)c1ccc(-c2cnc(C3CCCN3C(=O)Nc3ccc(C4CC4)c(C(F)(F)F)c3)c(F)c2)cc1 ; O=C(O)c1ccc(-c2cnc([C@@H]3CCCN3C(=O)Nc3ccc(C4CC4)c(C(F)(F)F)c3)c(F)c2)cc1 ; O=C(O)c1ccc(-c2cnc([C@H]3CCCN3C(=O)Nc3ccc(C4CC4)c(C(F)(F)F)c3)c(F)c2)cc1]

4-(6-(1-((4-cyclopropyl-3-(trifluoromethyl)phenyl)carbamoyl)pyrrolidin-2-yl)-5-fluoropyridin-3-yl)benzoic acid (125 mg) was separated by prep Chiral-HPLC (Column: CHIRAL ART Cellulose-SC, 3*25 cm, 5 μm; Mobile Phase A: Hexane (0.1% FA), Mobile Phase B: EtOH; Flow rate: 40 mL/min; Gradient (B %): isocratic 20% B; Wave Length: 252/286 nm; RT1 (min): 7.10; RT2 (min): 8.50) to afford (Compound 58a; Method W, Peak 1, 0.91 min) (S)-4-(6-(1-((4-cyclopropyl-3-(trifluoromethyl)phenyl)carbamoyl)pyrrolidin-2-yl)-5-fluoropyridin-3-yl)benzoic acid (35.2 mg) as a white solid and (Compound 58b; Method W, Peak 2, 1.14 min) (R)-4-(6-(1-((4-cyclopropyl-3-(trifluoromethyl)phenyl)carbamoyl)pyrrolidin-2-yl)-5-fluoropyridin-3-yl)benzoic acid (28.4 mg) as a white solid.

| Cmpd # | Characterization Data |
|---|---|
| 58a | LCMS (ESI) [M + H]⁺: 514.2 |
| | ¹H NMR (400 MHz, DMSO-d₆) δ 13.09 (bs, 1H), 8.73 (s, 1H), 8.50 (s, 1H), 8.09-8.02 (m, 3H), |
| | 7.90-7.86 (m, 3H), 7.61 (d, J = 8.4 Hz, 1H), 6.99 (d, J = 8.8 Hz, 1H), 5.36-5.33 (m, 1H), 3.79- |
| | 3.74 (m, 1H), 3.66-3.62 (m, 1H), 2.38-2.33 (m, 1H), 2.16-1.99 (m, 3H), 1.92-1.88 (m, 1H), |
| | 0.92-0.90 (m, 2H), 0.70-0.66 (m, 2H). |
| 58b | LCMS (ESI) [M + H]⁺: 514.2 |
| | ¹H NMR (400 MHz, DMSO-d₆) δ 13.09 (bs, 1H), 8.73 (s, 1H), 8.50 (s, 1H), 8.09-8.02 (m, 3H), |
| | 7.90-7.86 (m, 3H), 7.61 (d, J = 8.4 Hz, 1H), 6.99 (d, J = 8.8 Hz, 1H), 5.36-5.33 (m, 1H), 3.79- |
| | 3.74 (m, 1H), 3.66-3.62 (m, 1H), 2.38-2.33 (m, 1H), 2.16-1.99 (m, 3H), 1.92-1.88 (m, 1H), |
| | 0.92-0.90 (m, 2H), 0.70-0.66 (m, 2H). |

### Example 59: (S)-4-(5-fluoro-6-(1-((3-fluoro-4-isopropylphenyl)carbamoyl)pyrrolidin-2-yl)pyridin-3-yl)benzoic acid & (R)-4-(5-fluoro-6-(1-((3-fluoro-4-isopropylphenyl)carbamoyl)pyrrolidin-2-yl)pyridin-3-yl)benzoic acid

### Step 1: 3-fluoro-4-(prop-1-en-2-yl)aniline

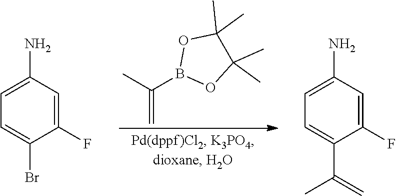

A mixture of 4-bromo-3-fluoroaniline (150 mg, 0.7 mmol) and 4,4,5,5-tetramethyl-2-(prop-1-en-2-yl)-1,3,2-dioxaborolane (0.27 g, 1.5 mmol), Pd(dppf)Cl₂ (57 mg, 79 μmol), K₃PO₄ (500 mg, 2.3 mmol) in dioxane (1 mL) and H₂O (0.1 mL) was stirred at 80° C. for 2 hours under nitrogen atmosphere. Upon completion, the resulting mixture was concentrated under reduced pressure. The crude residue was purified by silica gel column chromatography, eluted with PE/EA (1:1) to afford 3-fluoro-4-(prop-1-en-2-yl)aniline (110 mg, 92% yield) as a yellow liquid. LCMS (ESI) [M+H]⁺: 152

### Step 2: 3-fluoro-4-isopropylaniline

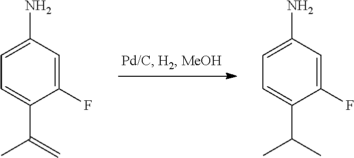

A mixture of 3-fluoro-4-(prop-1-en-2-yl)aniline (110 mg, 0.7 mmol) and Pd/C (77 mg, 0.7 mmol, 10 wt %) in MeOH (10 mL) was stirred at room temperature for 4 hours under hydrogen atmosphere. Upon completion, the resulting mixture was filtered over celite. The filter cake was washed with MeOH (3×10 mL). The filtrate was concentrated under reduced pressure. The crude residue was purified by silica gel column chromatography, eluted with PE/EA (1:1) to afford 3-fluoro-4-isopropylaniline (90 mg, 80% yield) as a yellow liquid. LCMS (ESI) [M+H]⁺: 154

### Step 3: 4-(5-fluoro-6-(1-((3-fluoro-4-isopropylphenyl)carbamoyl)pyrrolidin-2-yl)pyridin-3-yl)benzoic acid

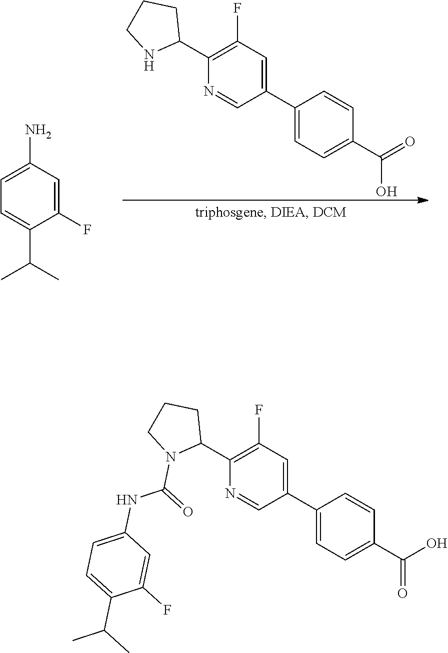 [SMILES: CC(C)c1ccc(N)cc1F ; O=C(O)c1ccc(-c2cnc(C3CCCN3)c(F)c2)cc1 ; CC(C)c1ccc(NC(=O)N2CCCC2c2ncc(-c3ccc(C(=O)O)cc3)cc2F)cc1F]

A solution of triphosgene (61 mg, 0.2 mmol) in DCM (4 mL) was treated with 3-fluoro-4-isopropylaniline (80 mg, 0.5 mmol) and DIEA (337 mg, 2.6 mmol) at 0° C. under nitrogen atmosphere. After 10 minutes, 4-(5-fluoro-6-(pyrrolidin-2-yl)pyridin-3-yl)benzoic acid (149 mg, 0.5 mmol) was added dropwise at 0° C. The resulting mixture was warmed to room temperature and stirred for 2 hours. Upon completion, the reaction was quenched with ice water at 0° C. The resulting mixture was extracted with EtOAc (3×30 mL). The combined organic layers were washed with water (3×5 mL), dried over anhydrous Na₂SO₄, filtered and concentrated under reduced pressure. The crude residue was purified by silica gel column chromatography, eluted with CH₂Cl₂/MeOH (1:1) to afford 4-(5-fluoro-6-(1-((3-fluoro-4-isopropylphenyl)carbamoyl)pyrrolidin-2-yl)pyridin-3-yl)benzoic acid (100 mg, 41% yield) as a light yellow solid. LCMS (ESI) [M+H]⁺: 466

### Step 4: (S)-4-(5-fluoro-6-(1-((3-fluoro-4-isopropylphenyl)carbamoyl)pyrrolidin-2-yl)pyridin-3-yl)benzoic acid & (R)-4-(5-fluoro-6-(1-((3-fluoro-4-isopropylphenyl)carbamoyl)pyrrolidin-2-yl)pyridin-3-yl)benzoic acid

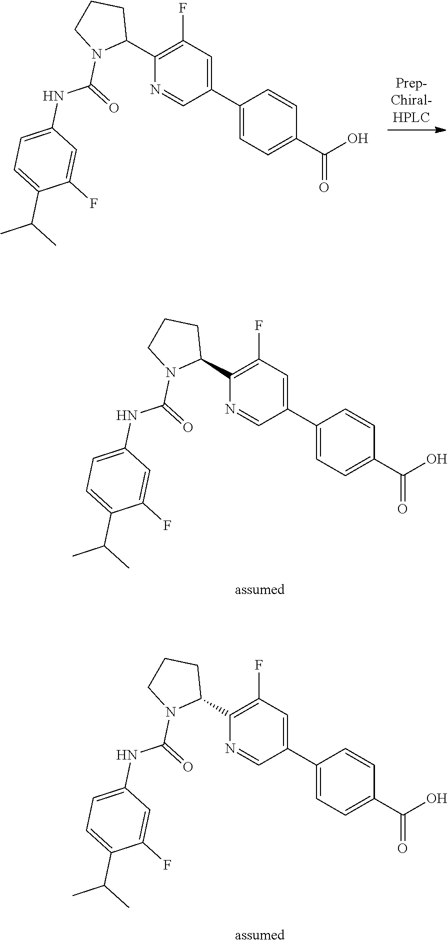 [SMILES: CC(C)c1ccc(NC(=O)N2CCCC2c2ncc(-c3ccc(C(=O)O)cc3)cc2F)cc1F ; CC(C)c1ccc(NC(=O)N2CCC[C@H]2c2ncc(-c3ccc(C(=O)O)cc3)cc2F)cc1F ; CC(C)c1ccc(NC(=O)N2CCC[C@@H]2c2ncc(-c3ccc(C(=O)O)cc3)cc2F)cc1F]

4-(5-fluoro-6-(1-((3-fluoro-4-isopropylphenyl)carbamoyl)pyrrolidin-2-yl)pyridin-3-yl)benzoic acid (100 mg) was separated by Prep-Chiral-HPLC (conditions: Column: CHIRALPAK IK 3*25 cm, 5 μm; Mobile Phase A: Hexane (0.1% formic acid), Mobile Phase B: EtOH; Flow rate: 40 mL/min; Gradient (B %): isocratic 25% B; Wave Length: 244/210 nm; RT1 (min): 6.37; RT2 (min): 9.65) to afford first peak (Compound 59a; Method G, Peak 1, 0.79 min) (S)-4-(5-fluoro-6-(1-((3-fluoro-4-isopropylphenyl)carbamoyl)pyrrolidin-2-yl)pyridin-3-yl)benzoic acid (36.4 mg, 38% yield) and second peak (Compound 59b; Method G, Peak 2, 1.01 min) (R)-4-(5-fluoro-6-(1-((3-fluoro-4-isopropylphenyl)carbamoyl)pyrrolidin-2-yl)pyridin-3-yl)benzoic acid (33.6 mg, 35% yield) as a white solid.

| Cmpd # | Characterization Data |
|---|---|
| 59a | LCMS (ESI) [M + H]⁺: 466.2 |
| | ¹H NMR (400 MHz, DMSO-d₆) δ 8.73 (t, J = 1.6 Hz, 1H), 8.37 (s, 1H), 8.09-8.02 (m, 3H), 7.90- |
| | 7.86 (m, 2H), 7.35 (d, J = 12 Hz, 1H), 7.19-7.09 (m, 2H), 5.34 (d, J = 11.6 Hz, 1H), 3.77-3.74 |
| | (m, 1H), 3.64-3.61 (m, 1H), 3.04-3.02 (m, 1H), 2.36-2.33 (m, 1H), 2.11-2.01 (m, 2H), 1.92- |
| | 1.89 (m, 1H), 1.15 (d, J = 6.8 Hz, 6H) |
| 59b | LCMS (ESI) [M + H]⁺: 466.2 |
| | ¹H NMR (400 MHz, DMSO-d₆) δ 8.73 (t, J = 1.6 Hz, 1H), 8.37 (s, 1H), 8.09-8.02 (m, 3H), 7.90- |
| | 7.86 (m, 2H), 7.35 (d, J = 12 Hz, 1H), 7.19-7.09 (m, 2H), 5.34 (d, J = 11.6 Hz, 1H), 3.77-3.74 |
| | (m, 1H), 3.64-3.61 (m, 1H), 3.04-3.02 (m, 1H), 2.36-2.33 (m, 1H), 2.11-2.01 (m, 2H), 1.92- |
| | 1.89 (m, 1H), 1.15 (d, J = 6.8 Hz, 6H) |

### Example 61: (S)-4-(2-(1-((4-isopropylphenyl)carbamoyl)pyrrolidin-2-yl)benzo[d]oxazol-5-yl)benzoic acid and (R)-4-(2-(1-((4-isopropylphenyl)carbamoyl)pyrrolidin-2-yl)benzo[d]oxazol-5-yl)benzoic acid

### Step 1: tert-butyl (R)-2-((5-bromo-2-hydroxyphenyl)carbamoyl)pyrrolidine-1-carboxylate

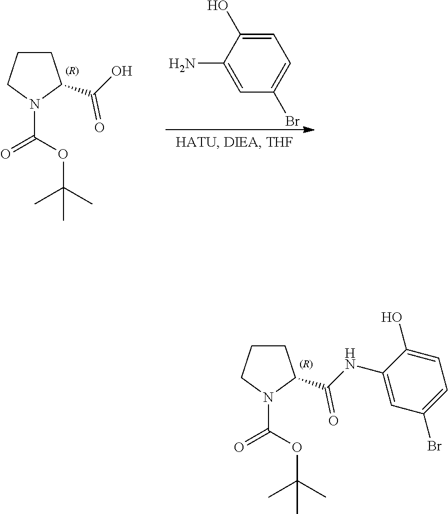

To a mixture of (tert-butoxycarbonyl)-D-proline (1.0 g, 4.6 mmol) and HATU (2.2 g, 5.5 mmol) in THF (20 mL) was treated with DIEA (0.66 g, 5.1 mmol) at 0° C. under nitrogen atmosphere. After 30 minutes, 2-amino-4-bromophenol (0.96 g, 5.1 mmol) was added dropwise. The resulting mixture was warmed to room temperature and stirred for 4 hours. Upon completion, the resulting mixture was concentrated under reduced pressure. The crude residue was purified by silica gel column chromatography, eluted with PE/EA (3/1) to afford tert-butyl (R)-2-((5-bromo-2-hydroxyphenyl)carbamoyl)pyrrolidine-1-carboxylate (1.5 g, 84% yield) as a yellow solid. LCMS (ESI) [M+H]⁺: 385

### Step 2: tert-butyl (R)-2-(5-bromobenzo[d]oxazol-2-yl)pyrrolidine-1-carboxylate

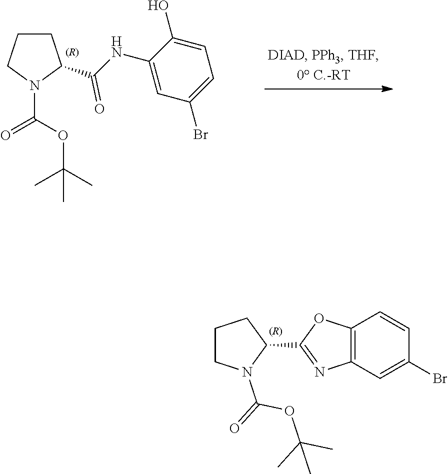

To a mixture of tert-butyl (R)-2-((5-bromo-2-hydroxyphenyl)carbamoyl)pyrrolidine-1-carboxylate (1.5 g, 3.9 mmol) and PPh₃ (1.0 g, 3.9 mmol) in THF (15 mL) was added DIAD (0.78 g, 3.9 mmol) dropwise at 0° C. under nitrogen atmosphere. The resulting mixture was stirred at room temperature for 1 hour. Upon completion, the resulting mixture was diluted with water (20 mL). The resulting mixture was extracted with EtOAc (3×30 mL). The combined organic layers were dried over anhydrous Na₂SO₄, filtered and concentrated under reduced pressure. The crude residue was purified by silica gel column chromatography, eluted with PE/EA (3/1) to afford tert-butyl (R)-2-(5-bromobenzo[d]oxazol-2-yl)pyrrolidine-1-carboxylate (750 mg, 52% yield) as an off-white solid. LCMS (ESI) [M+H]⁺: 367

### Step 3: tert-butyl (R)-2-(5-(4-(tert-butoxycarbonyl)phenyl)benzo[d]oxazol-2-yl)pyrrolidine-1-carboxylate

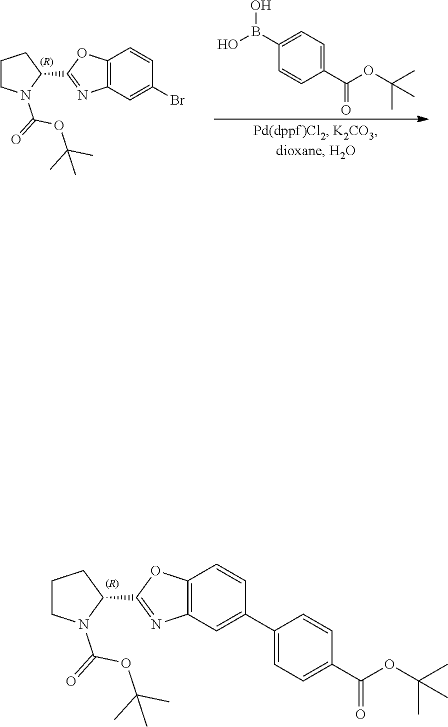

To a mixture of tert-butyl (R)-2-(5-bromobenzo[d]oxazol-2-yl)pyrrolidine-1-carboxylate (350 mg, 0.95 mmol), 4-(tert-butoxycarbonyl)phenylboronic acid (423 mg, 1.9 mmol), Pd(dppf)Cl₂ (156 mg, 0.2 mmol) and K₂CO₃ (393 mg, 2.85 mmol) in dioxane (4 mL) and H₂O (1 mL) was stirred at 90° C. for 1 hour under nitrogen atmosphere. Upon completion, the resulting mixture was concentrated under reduced pressure. The crude residue was purified by reversed-phase flash (conditions: column: C18 silica gel; mobile phase A: water (0.1% formic acid), Mobile Phase B: MeCN; Gradient (B %): 0% to 100% in 20 min; detector: UV 254 nm) to afford tert-butyl (R)-2-(5-(4-(tert-butoxycarbonyl)phenyl)benzo[d]oxazol-2-yl)pyrrolidine-1-carboxylate (300 mg, 68% yield) as a white solid. LCMS (ESI) [M+H]⁺: 465

Step 4: 4-(2-(pyrrolidin-2-yl)benzo[d]oxazol-5-yl)benzoic acid

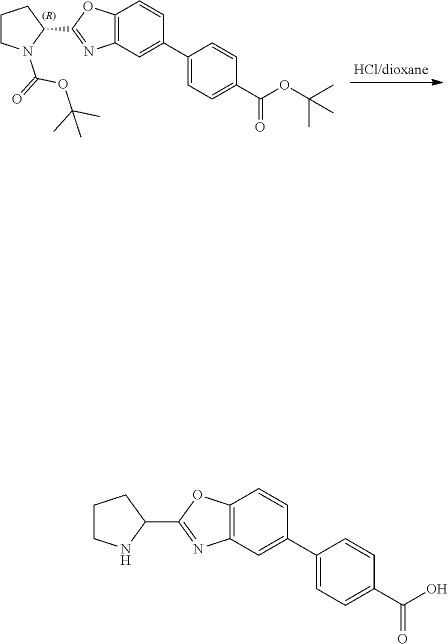

To a mixture of tert-butyl (R)-2-(5-(4-(tert-butoxycarbonyl)phenyl)benzo[d]oxazol-2-yl)pyrrolidine-1-carboxylate (220 mg, 0.47 mmol) in 4.0 M HCl in 1,4-dioxane (3 mL) was stirred at room temperature overnight. Upon completion, the resulting mixture was concentrated under reduced pressure to afford 4-(2-(pyrrolidin-2-yl)benzo[d]oxazol-5-yl)benzoic acid (180 mg, crude racemate) as an off-white solid, which was used directly in the next step without further purification. LCMS (ESI) [M+H]⁺: 309

### Step 5: 4-(2-(1-((4-isopropylphenyl)carbamoyl)pyrrolidin-2-yl)benzo[d]oxazol-5-yl)benzoic acid

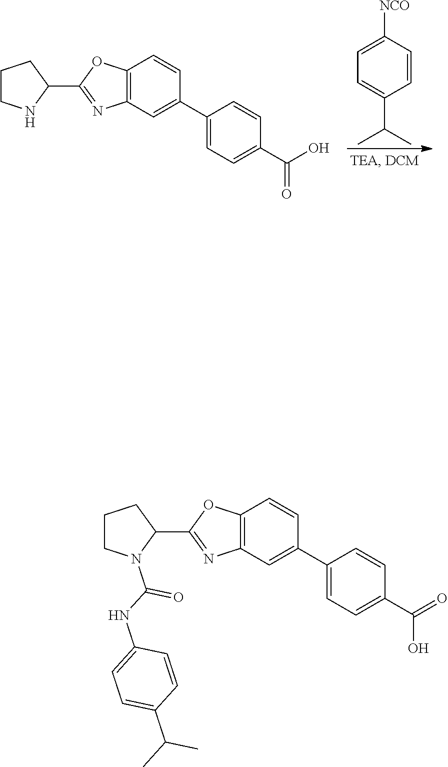

To a mixture of 4-(2-(pyrrolidin-2-yl)benzo[d]oxazol-5-yl)benzoic acid (180 mg, 0.6 mmol) and Et₃N (59.1 mg, 0.6 mmol) in DCM (3 mL) was added 1-isocyanato-4-isopropylbenzene (94.1 mg, 0.6 mmol) dropwise at 0° C. under nitrogen atmosphere. The resulting mixture was stirred at room temperature for 1 hour. Upon completion, the resulting mixture was concentrated under reduced pressure. The crude residue was purified by reversed-phase flash chromatography (conditions: column: C18 silica gel; mobile phase A: water (0.1% formic acid), Mobile Phase B: MeCN; Gradient (B %): 0% to 100% in 20 min; detector: UV 254 nm) to afford 4-(2-(1-((4-isopropylphenyl)carbamoyl)pyrrolidin-2-yl)benzo[d]oxazol-5-yl)benzoic acid (120 mg, 44% yield) as a white solid. LCMS (ESI) [M+H]⁺: 470

### Step 6: (S)-4-(2-(1-((4-isopropylphenyl)carbamoyl)pyrrolidin-2-yl)benzo[d]oxazol-5-yl)benzoic acid and (R)-4-(2-(1-((4-isopropylphenyl)carbamoyl)pyrrolidin-2-yl)benzo[d]oxazol-5-yl)benzoic acid

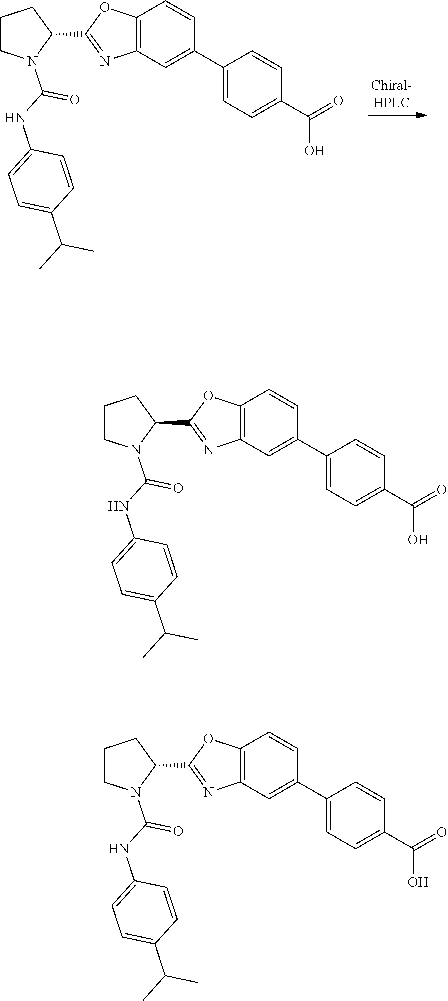 [SMILES: CC(C)c1ccc(NC(=O)N2CCC[C@@H]2c2nc3cc(-c4ccc(C(=O)O)cc4)ccc3o2)cc1 ; CC(C)c1ccc(NC(=O)N2CCC[C@H]2c2nc3cc(-c4ccc(C(=O)O)cc4)ccc3o2)cc1 ; CC(C)c1ccc(NC(=O)N2CCC[C@@H]2c2nc3cc(-c4ccc(C(=O)O)cc4)ccc3o2)cc1]

The 4-(2-(1-((4-isopropylphenyl)carbamoyl)pyrrolidin-2-yl)benzo[d]oxazol-5-yl)benzoic acid (100 mg, 0.2 mmol) was purified by prep Chiral-HPLC (conditions: Column: CHIRAL ART Cellulose-SB, 3*25 cm, 5 μm; Mobile Phase A: Hexane (0.1% FA), Mobile Phase B: EtOH; Flow rate: 40 mL/min; Gradient (B %): isocratic 30% B; Wave Length: 254/220 nm; RT1 (min): 10.7; RT2 (min): 15.5) to afford first peak (Compound 61a; Method M, Peak 1, 1.05 min) (S)-4-(2-(1-((4-isopropylphenyl)carbamoyl)pyrrolidin-2-yl)benzo[d]oxazol-5-yl)benzoic acid (19.2 mg) as a white solid and second peak (Compound 61b; Method M, Peak 2, 1.47 min) (R)-4-(2-(1-((4-isopropylphenyl)carbamoyl)pyrrolidin-2-yl)benzo[d]oxazol-5-yl)benzoic acid (44.1 mg) as a white solid.

| Cmpd # | Characterization Data |
|---|---|
| 61a | LCMS (ESI) [M + H]⁺: 470.2 |
| | ¹H NMR (400 MHz, DMSO-d₆) δ 8.35 (bs, 1H), 8.03 (bs, 3H), 7.81-7.72 (bm, 4H), 7.37 (bs, 2H), |
| | 7.08 (bs, 2H), 5.27 (bs, 1H), 3.79 (bs, 1H), 3.61 (bs, 1H), 2.79 (bs, 1H), 2.40 (bs, 1H), 2.11 (bs, 3H), |
| | 1.15 (bs, 6H). |
| 61b | LCMS (ESI) [M + H]⁺: 470.2 |
| | ¹H NMR (400 MHz, DMSO-d₆) δ 8.35 (s, 1H), 8.02-8.00 (m, 3H), 7.80-7.78 (m, 3H), 7.71 (d, J = |
| | 8.4 Hz, 1H), 7.36 (d, J = 8.0 Hz, 2H), 7.07 (d, J = 8.1 Hz, 2H), 5.29-5.23 (m, 1H), 3.78-3.77 |
| | (m, 1H), 3.61-3.59 (m, 1H), 2.80-2.73 (m, 1H), 2.50-2.39 (m, 1H), 2.13-2.08 (m, 3H), 1.30 |
| | (d, J = 6.8 Hz, 6H). |

### Example 64: (S)-4-(5-fluoro-6-(1-((5-isopropyl-4-methylpyridin-2-yl)carbamoyl)pyrrolidin-2-yl)pyridin-3-yl)benzoic acid & (R)-4-(5-fluoro-6-(1-((5-isopropyl-4-methylpyridin-2-yl)carbamoyl)pyrrolidin-2-yl)pyridin-3-yl)benzoic acid

### Step 1: 4-methyl-5-(prop-1-en-2-yl)pyridin-2-amine

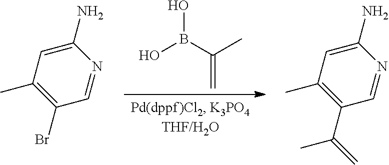

To a stirred solution of 5-bromo-4-methylpyridin-2-amine (1.0 g, 5.35 mmol) and prop-1-en-2-ylboronic acid (1.38 g, 16.03 mmol) in THF (10 mL) were added Pd(dppf)Cl₂ (391 mg, 0.535 mmol), H₂O (2 mL), and K₃PO₄ (3.40 g, 16.04 mmol) at room temperature under nitrogen atmosphere. The resulting mixture was stirred at 80° C. for 2 hours. Upon completion, the mixture was allowed to cool down to room temperature. The resulting mixture was poured into water (100 mL) and extracted with EtOAc (3×100 mL). The combined organic layers were washed with water (3×100 mL), dried over anhydrous Na₂SO₄, filtered and concentrated under reduced pressure. The crude residue was purified by silica gel column chromatography, eluted with PE/EA (1:1) to afford 4-methyl-5-(prop-1-en-2-yl)pyridin-2-amine (500 mg, 63% yield) as a yellow oil. LCMS (ESI) [M+H]⁺: 149.1

### Step 2: 5-isopropyl-4-methylpyridin-2-amine

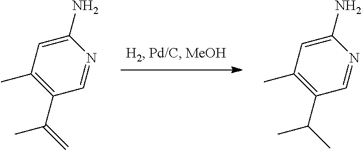

A mixture of 4-methyl-5-(prop-1-en-2-yl)pyridin-2-amine (500 mg, 3.37 mmol) and Pd/C (359 mg, 0.337 mmol, 10% on charcoal) in methanol (20 mL) was stirred at room temperature for 2 hours under hydrogen atmosphere. Upon completion, the resulting mixture was filtered over celite. The filter cake was washed with methanol (3×10 mL). The filtrate was concentrated under reduced pressure to afford 5-isopropyl-4-methylpyridin-2-amine (400 mg, crude) as a yellow oil, which was used directly in the next step without further purification. LCMS (ESI) [M+H]⁺: 151.1

### Step 3: methyl 4-(5-fluoro-6-(1-((5-isopropyl-4-methylpyridin-2-yl)carbamoyl)pyrrolidin-2-yl)pyridin-3-yl)benzoate

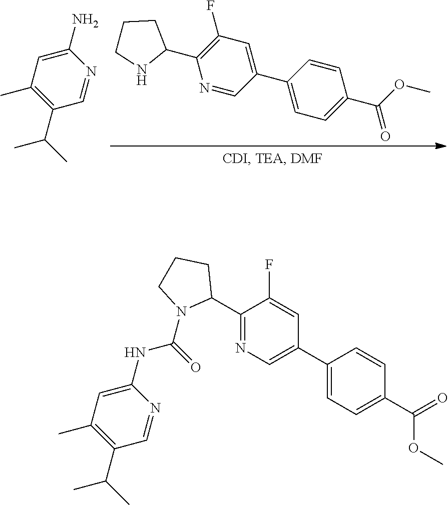

A solution of methyl 4-[5-fluoro-6-(pyrrolidin-2-yl)pyridin-3-yl]benzoate (300 mg, 1.11 mmol), 5-isopropyl-4-methylpyridin-2-amine (500 mg, 3.33 mmol), CDI (900 mg, 5.5 mmol) and triethylamine (500 mg, 5.5 mmol) in DMF (3 mL) was stirred at 60° C. for 1 hour. Upon completion, the mixture was allowed to cool down to room temperature and then was concentrated under reduced pressure. The crude residue directly was purified by reversed-phase flash chromatography (conditions: column: C18 silica gel; mobile phase A: Water (0.1% FA), mobile phase B: MeCN; Gradient (B %): 10% to 50% in 10 min; detector: UV 254 nm) to afford methyl 4-(5-fluoro-6-(1-((5-isopropyl-4-methylpyridin-2-yl)carbamoyl)pyrrolidin-2-yl)pyridin-3-yl)benzoate (200 mg) as a yellow oil. LCMS (ESI) [M+H]⁺: 477.2

### Step 4: 4-(5-fluoro-6-(1-((5-isopropyl-4-methylpyridin-2-yl)carbamoyl)pyrrolidin-2-yl)pyridin-3-yl)benzoic acid

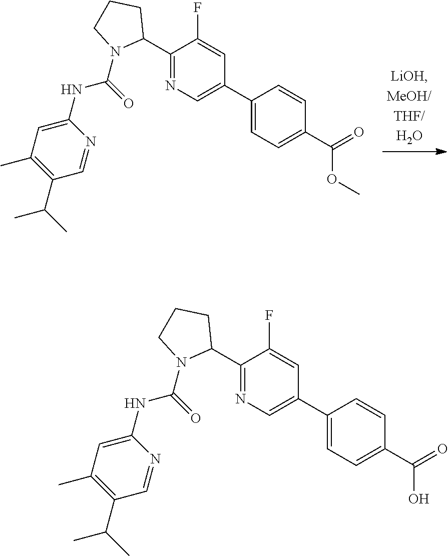

To a stirred solution of methyl 4-(5-fluoro-6-(1-((5-isopropyl-4-methylpyridin-2-yl)carbamoyl)pyrrolidin-2-yl)pyridin-3-yl)benzoate (200 mg, 0.420 mmol) and LiOH monohydrate (84 mg, 2.10 mmol) in THF (2 mL) was added H₂O (0.5 mL) under nitrogen atmosphere. The resulting mixture was stirred at 50° C. for 2 hours. Upon completion, the mixture was allowed to cool down to room temperature and then was concentrated under reduced pressure. The crude residue was directly purified by reversed-phase flash chromatography (conditions: column: C18 silica gel; mobile phase A: Water (0.1% FA), mobile phase B: MeCN; Gradient (B %): 10% to 70% in 30 min; detector: UV 254 nm) to afford 4-(5-fluoro-6-(1-((5-isopropyl-4-methylpyridin-2-yl)carbamoyl)pyrrolidin-2-yl)pyridin-3-yl)benzoic acid (100 mg, 52% yield) as a white solid. LCMS (ESI) [M+H]⁺: 463.2

### Step 5: (S)-4-(5-fluoro-6-(1-((5-isopropyl-4-methylpyridin-2-yl)carbamoyl)pyrrolidin-2-yl)pyridin-3-yl)benzoic acid & (R)-4-(5-fluoro-6-(1-((5-isopropyl-4-methylpyridin-2-yl)carbamoyl)pyrrolidin-2-yl)pyridin-3-yl)benzoic acid

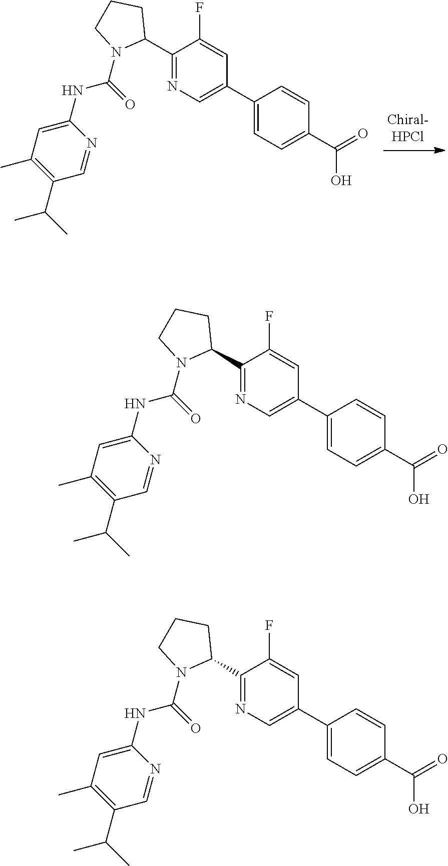 [SMILES: Cc1cc(NC(=O)N2CCCC2c2ncc(-c3ccc(C(=O)O)cc3)cc2F)ncc1C(C)C ; Cc1cc(NC(=O)N2CCC[C@H]2c2ncc(-c3ccc(C(=O)O)cc3)cc2F)ncc1C(C)C ; Cc1cc(NC(=O)N2CCC[C@@H]2c2ncc(-c3ccc(C(=O)O)cc3)cc2F)ncc1C(C)C]

4-(5-fluoro-6-{1-[(5-isopropyl-4-methylpyridin-2-yl)carbamoyl]pyrrolidin-2-yl}pyridin-3-yl)benzoic acid (100 mg) was separated by prep Chiral-HPLC (conditions: Column: CHIRAL ART Cellulose-SC, 3*25 cm, 5 μm; Mobile Phase A: Hexane (0.1% FA), Mobile Phase B: EtOH; Flow rate: 40 mL/min; Gradient (B %): isocratic 40% B; Wave Length: 220/254 nm; RT1 (min): 16.1; RT2 (min): 19) to afford (Compound 64a; Method Z, Peak 1, 1.07 min) (S)-4-(5-fluoro-6-(1-((5-isopropyl-4-methylpyridin-2-yl)carbamoyl)pyrrolidin-2-yl)pyridin-3-yl)benzoic acid (1^{st} peak, 23.1 mg, 23% yield) as a white solid and (Compound 64b; Method Z, Peak 2, 1.42 min) (R)-4-(5-fluoro-6-(1-((5-isopropyl-4-methylpyridin-2-yl)carbamoyl)pyrrolidin-2-yl)pyridin-3-yl)benzoic acid (2^{nd} peak, 23.9 mg, 24% yield) as a white solid.

| Cmpd # | Characterization Data |
|---|---|
| 64a | LCMS (ESI) [M + H]⁺: 463.3 |
| | ¹H NMR (400 MHz, DMSO-d₆) δ 13.01 (bs, 1H), 8.74 (s, 1H), 8.51 (bs, 1H), 8.08 (dd, J = 11.5, 1.9 |
| | Hz, 1H), 8.07-7.99 (m, 3H), 7.89 (d, J = 8.2 Hz, 2H), 7.55 (s, 1H), 5.36 (bs, 1H), 3.85-3.72 (m, |
| | 1H), 3.71-3.55 (m, 1H), 3.00 (hept, J = 6.9 Hz, 1H), 2.37-2.30 (m, 1H), 2.21 (s, 3H), 2.14-2.04 |
| | (m, 1H), 2.02-1.93 (m, 1H), 1.93-1.82 (m, 1H), 1.18 (dd, J = 6.9, 2.5 Hz, 6H). |
| 64b | LCMS (ESI) [M + H]⁺: 463.3 |
| | ¹H NMR (400 MHz, DMSO-d₆) δ 12.99 (bs, 1H), 8.74 (s, 1H), 8.51 (bs, 1H), 8.08 (dd, J = 11.5, 1.9 |
| | Hz, 1H), 8.07-7.98 (m, 3H), 7.89 (d, J = 8.4 Hz, 2H), 7.55 (s, 1H), 5.36 (bs, 1H), 3.84-3.72 (m, |
| | 1H), 3.69-3.59 (m, 1H), 3.07-2.90 (m, 1H), 2.34-2.30 (m, 1H), 2.21 (s, 3H), 2.15-2.03 (m, |
| | 1H), 2.03-1.95 (m, 1H), 1.95-1.84 (m, 1H), 1.18 (dd, J = 6.9, 2.5 Hz, 6H). |

### Example 65: (S)-4-(6-(1-((4-cyclopropyl-3-fluorophenyl)carbamoyl)pyrrolidin-2-yl)-5-fluoropyridin-3-yl)benzoic acid & (R)-4-(6-(1-((4-cyclopropyl-3-fluorophenyl)carbamoyl)pyrrolidin-2-yl)-5-fluoropyridin-3-yl)benzoic acid

### Step 1: 4-cyclopropyl-3-fluoroaniline

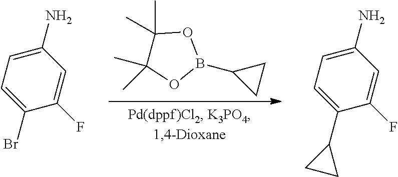

A mixture of 4-bromo-3-fluoroaniline (500 mg, 2.63 mmol), cyclopropylboronic acid (452 mg, 5.26 mmol), Pd(dppf)Cl₂ (215 mg, 0.263 mmol) and potassium phosphate tribasic (1.68 g, 7.89 mmol) in 1,4-dioxane (6 mL) was stirred at 90° C. for 1 hour under nitrogen atmosphere. Upon completion, the mixture was concentrated under reduced pressure. The crude residue was directly purified by silica gel chromatography, eluted with Petroleum ether/Ethyl acetate=15:1 to give 4-cyclopropyl-3-fluoroaniline (380 mg, 96% yield) as a brown oil. LCMS (ESI) [M+H]⁺: 152.2

### Step 2: methyl 4-(6-(1-((4-cyclopropyl-3-fluorophenyl)carbamoyl)pyrrolidin-2-yl)-5-fluoropyridin-3-yl)benzoate

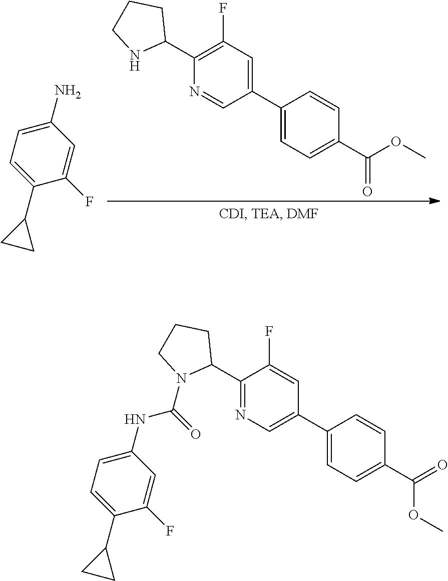 [SMILES: Nc1ccc(C2CC2)c(F)c1 ; COC(=O)c1ccc(-c2cnc(C3CCCN3)c(F)c2)cc1 ; COC(=O)c1ccc(-c2cnc(C3CCCN3C(=O)Nc3ccc(C4CC4)c(F)c3)c(F)c2)cc1]

A mixture of 4-cyclopropyl-3-fluoroaniline (302 mg, 2.00 mmol), methyl 4-[5-fluoro-6-(pyrrolidin-2-yl)pyridin-3-yl]benzoate (200 mg, 0.67 mmol), CDI (54 mg, 3.330 mmol) and triethylamine (337 mg, 3.33 mmol) in DMF (4 mL) was stirred at room temperature for 1 hour. Upon completion, the mixture was concentrated under reduced pressure. The crude residue was directly purified by reversed phase flash chromatography [conditions: column C18 silica gel; Mobile Phase A: Water (0.1% FA), Mobile Phase B: acetonitrile; Gradient: 0% B to 100% B in 20 min; detector: UV 254 nm) to afford methyl 4-(6-(1-((4-cyclopropyl-3-fluorophenyl)carbamoyl)pyrrolidin-2-yl)-5-fluoropyridin-3-yl)benzoate (80 mg, 25% yield) as an orange oil. LCMS (ESI) [M+H]⁺: 478.5

### Step 3: 4-(6-(1-((4-cyclopropyl-3-fluorophenyl)carbamoyl)pyrrolidin-2-yl)-5-fluoropyridin-3-yl)benzoic acid

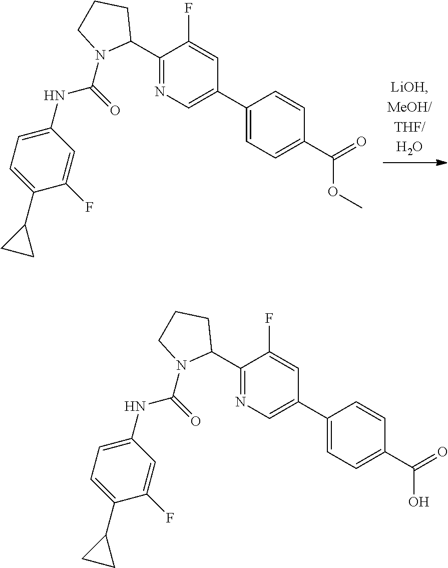 [SMILES: COC(=O)c1ccc(-c2cnc(C3CCCN3C(=O)Nc3ccc(C4CC4)c(F)c3)c(F)c2)cc1 ; O=C(O)c1ccc(-c2cnc(C3CCCN3C(=O)Nc3ccc(C4CC4)c(F)c3)c(F)c2)cc1]

A mixture of methyl 4-(6-(1-((4-cyclopropyl-3-fluorophenyl)carbamoyl)pyrrolidin-2-yl)-5-fluoropyridin-3-yl)benzoate (80 mg, 0.168 mmol), lithium hydroxide monohydrate (35 mg, 0.84 mmol) in tetrahydrofuran (1 mL), methanol (1 mL) and water (1 mL) was stirred at room temperature for 1 hour. Upon completion, the mixture was concentrated under reduced pressure. The crude residue was directly purified by reversed phase flash chromatography (conditions: column C18 silica gel; Mobile Phase A: Water (0.1% FA), Mobile Phase B: acetonitrile; Gradient: 0% B to 100% B in 25 min; detector: UV 254 nm) to afford 4-(6-(1-((4-cyclopropyl-3-fluorophenyl)carbamoyl)pyrrolidin-2-yl)-5-fluoropyridin-3-yl)benzoic acid (45 mg, 58% yield) as an off-white solid. LCMS (ESI) [M+H]⁺: 464.5

### Step 4: (S)-4-(6-(1-((4-cyclopropyl-3-fluorophenyl)carbamoyl)pyrrolidin-2-yl)-5-fluoropyridin-3-yl)benzoic acid & (R)-4-(6-(1-((4-cyclopropyl-3-fluorophenyl)carbamoyl)pyrrolidin-2-yl)-5-fluoropyridin-3-yl)benzoic acid

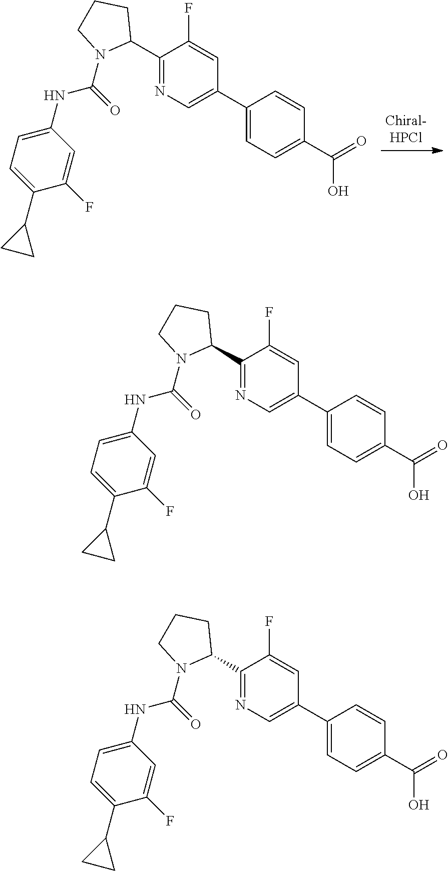 [SMILES: O=C(O)c1ccc(-c2cnc(C3CCCN3C(=O)Nc3ccc(C4CC4)c(F)c3)c(F)c2)cc1 ; O=C(O)c1ccc(-c2cnc([C@@H]3CCCN3C(=O)Nc3ccc(C4CC4)c(F)c3)c(F)c2)cc1 ; O=C(O)c1ccc(-c2cnc([C@H]3CCCN3C(=O)Nc3ccc(C4CC4)c(F)c3)c(F)c2)cc1]

4-(6-(1-((4-cyclopropyl-3-fluorophenyl)carbamoyl)pyrrolidin-2-yl)-5-fluoropyridin-3-yl)benzoic acid (45 mg, 0.173 mmol) was separated by prep Chiral-HPLC (conditions: Column: CHIRALPAK IK 3*25 cm, 5 μm; Mobile Phase A: Hexane (0.1% FA), Mobile Phase B: EtOH; Flow rate: 40 mL/min; Gradient (B %): isocratic 30% B; Wave Length: 248/285 nm; RT1 (min): 8.43; RT2 (min): 11.29) to afford (Compound 65a; Method G, Peak 1, 0.99 min) (S)-4-(6-(1-((4-cyclopropyl-3-fluorophenyl)carbamoyl)pyrrolidin-2-yl)-5-fluoropyridin-3-yl)benzoic acid (1^{st} peak, 11.8 mg, 15% yield) as a white solid and (Compound 65b; Method G, Peak 2, 1.32 min) (R)-4-(6-(1-((4-cyclopropyl-3-fluorophenyl)carbamoyl)pyrrolidin-2-yl)-5-fluoropyridin-3-yl)benzoic acid (2^{nd} peak, 11.8 mg, 15% yield) as a white solid.

| Cmpd # | Characterization Data |
|---|---|
| 65a | LCMS (ESI) [M + H]⁺: 464.1 |
| | ¹H NMR (400 MHz, DMSO-d₆) δ 13.02 (bs, 1H), 8.73 (s, 1H), 8.35 (s, 1H), 8.12-7.96 (m, 3H), |
| | 7.90 (d, J = 8.3 Hz, 2H), 7.35 (dd, J = 13.5, 2.1 Hz, 1H), 7.12 (dd, J = 8.5, 2.1 Hz, 1H), 6.80 (t, J = |
| | 8.8 Hz, 1H), 5.33 (dd, J = 8.2, 4.1 Hz, 1H), 3.81-3.69 (m, 1H), 3.65-3.55 (m, 1H), 2.39-2.27 |
| | (m, 1H), 2.17-1.95 (m, 2H), 1.95-1.83 (m, 2H), 0.91-0.81 (m, 2H), 0.64-0.56 (m, 2H). |
| 65b | LCMS (ESI) [M + H]⁺: 464.2 |
| | ¹H NMR (400 MHz, DMSO-d₆) δ 13.06 (bs, 1H), 8.73 (s, 1H), 8.35 (s, 1H), 8.10-7.95 (m, 3H), |
| | 7.89 (d, J = 8.1 Hz, 2H), 7.35 (dd, J = 13.5, 2.1 Hz, 1H), 7.12 (dd, J = 8.5, 2.1 Hz, 1H), 6.80 (t, J = |
| | 8.8 Hz, 1H), 5.33 (dd, J = 8.2, 4.1 Hz, 1H), 3.83-3.69 (m, 1H), 3.69-3.56 (m, 1H), 2.40-2.25 |
| | (m, 1H), 2.17-1.97 (m, 2H), 1.95-1.83 (m, 2H), 0.91-0.81 (m, 2H), 0.67-0.54 (m, 2H). |

### Example 66: (S)-4-(6-(1-((4-cyclopropyl-3-methylphenyl)carbamoyl)pyrrolidin-2-yl)-5-fluoropyridin-3-yl)benzoic acid & (R)-4-(6-(1-((4-cyclopropyl-3-methylphenyl)carbamoyl)pyrrolidin-2-yl)-5-fluoropyridin-3-yl)benzoic acid

### Step 1: 4-cyclopropyl-3-methylaniline

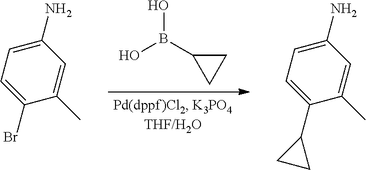

A solution of 3-methyl-4-bromoaniline (500 mg, 2.69 mmol) and cyclopropylboronic acid (462 mg, 5.37 mmol), Pd(dppf)Cl₂ (197 mg, 0.269 mmol) and K₃PO₄ (1.71 g, 8.06 mmol) in THF (5 mL) and water (1 mL) was stirred at 90° C. for 1 hour under nitrogen atmosphere. Upon completion, the resulting mixture was concentrated under reduced pressure. The crude residue was directly purified by reversed-phase flash chromatography (conditions: column: C18 silica gel; mobile phase A: Water (0.1% FA), mobile phase B: MeCN; Gradient (B %): 10% to 90% in 20 min; detector: UV 254 nm) to afford 4-cyclopropyl-3-methylaniline (200 mg, 51% yield) as a white solid. LCMS (ESI) [M+H]⁺: 148.2

### Step 2: methyl 4-(6-(1-((4-cyclopropyl-3-methylphenyl)carbamoyl)pyrrolidin-2-yl)-5-fluoropyridin-3-yl)benzoate

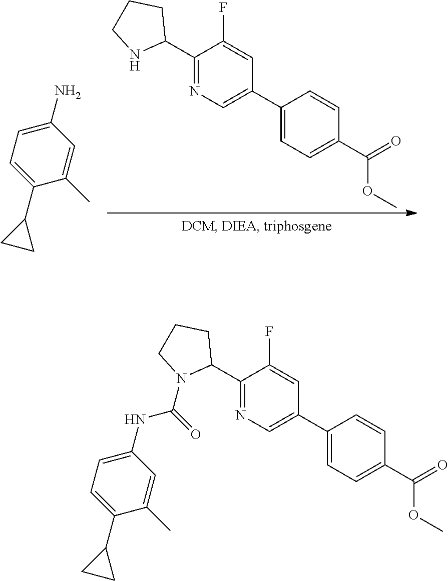 [SMILES: Cc1cc(N)ccc1C1CC1 ; COC(=O)c1ccc(-c2cnc(C3CCCN3)c(F)c2)cc1 ; COC(=O)c1ccc(-c2cnc(C3CCCN3C(=O)Nc3ccc(C4CC4)c(C)c3)c(F)c2)cc1]

To a solution of methyl 4-[5-fluoro-6-(pyrrolidin-2-yl)pyridin-3-yl]benzoate (200 mg, 0.666 mmol) and triphosgene (79 mg, 0.266 mmol) in DCM (5 mL) was added DIEA (258 mg, 2.00 mmol) and 4-cyclopropyl-3-methylaniline (147 mg, 1.00 mmol) at room temperature. The reaction mixture was stirred for 2 hours. Upon completion, the resulting mixture was concentrated under reduced pressure. The crude residue was directly purified by reversed-phase flash chromatography (conditions: column: C18 silica gel; mobile phase A: Water (0.1% FA), mobile phase B: MeCN; Gradient (B %): 10% to 80% in 23 min; detector: UV 254 nm) to afford methyl 4-(6-(1-((4-cyclopropyl-3-methylphenyl)carbamoyl)pyrrolidin-2-yl)-5-fluoropyridin-3-yl)benzoate (150 mg, 48% yield) as a yellow solid. LCMS (ESI) [M+H]⁺: 474.5

### Step 3: 4-(6-(1-((4-cyclopropyl-3-methylphenyl)carbamoyl)pyrrolidin-2-yl)-5-fluoropyridin-3-yl)benzoic acid

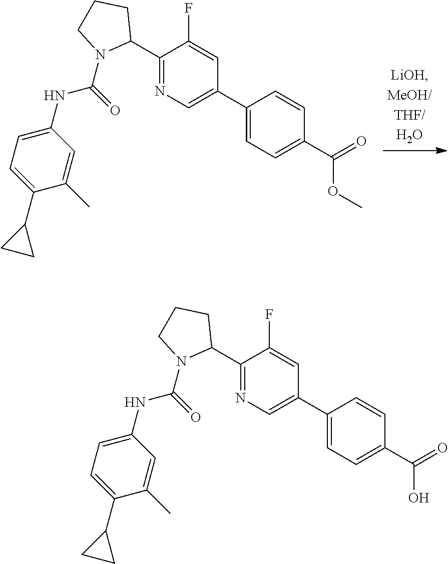 [SMILES: COC(=O)c1ccc(-c2cnc(C3CCCN3C(=O)Nc3ccc(C4CC4)c(C)c3)c(F)c2)cc1 ; Cc1cc(NC(=O)N2CCCC2c2ncc(-c3ccc(C(=O)O)cc3)cc2F)ccc1C1CC1]

A solution of methyl 4-(6-(1-((4-cyclopropyl-3-methylphenyl)carbamoyl)pyrrolidin-2-yl)-5-fluoropyridin-3-yl)benzoate (120 mg, 0.253 mmol) and LiOH monohydrate (53.3 mg, 1.27 mmol) in MeOH (2 mL), THF (2 mL) and H₂O (1 mL) was stirred at 50° C. for 1 hour. Upon completion, the resulting mixture was concentrated under reduced pressure. The crude residue was directly purified by reversed-phase flash chromatography (conditions: column: C18 silica gel; mobile phase A: Water (0.1% FA), mobile phase B: MeCN; Gradient (B %): 10% to 80% in 15 min; detector: UV 254 nm) to afford 4-(6-(1-((4-cyclopropyl-3-methylphenyl)carbamoyl)pyrrolidin-2-yl)-5-fluoropyridin-3-yl)benzoic acid (90 mg, 77% yield) as a white solid. LCMS (ESI) [M+H]⁺: 460.5

### Step 4: (S)-4-(6-(1-((4-cyclopropyl-3-methylphenyl)carbamoyl)pyrrolidin-2-yl)-5-fluoropyridin-3-yl)benzoic acid & (R)-4-(6-(1-((4-cyclopropyl-3-methylphenyl)carbamoyl)pyrrolidin-2-yl)-5-fluoropyridin-3-yl)benzoic acid

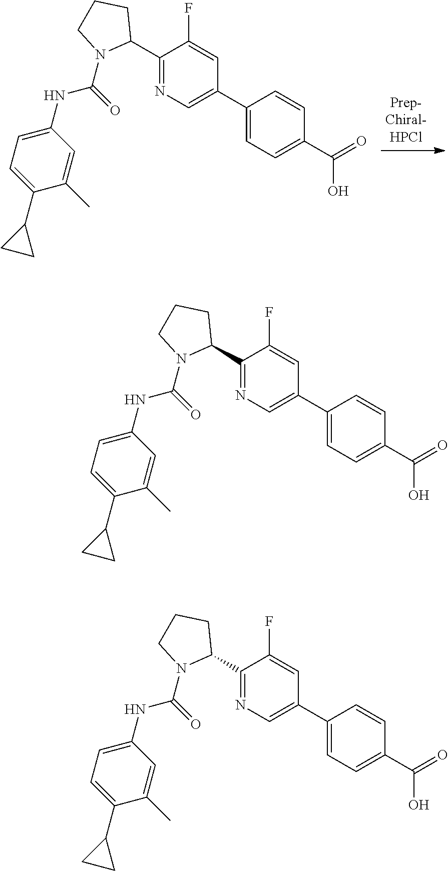 [SMILES: Cc1cc(NC(=O)N2CCCC2c2ncc(-c3ccc(C(=O)O)cc3)cc2F)ccc1C1CC1 ; Cc1cc(NC(=O)N2CCC[C@H]2c2ncc(-c3ccc(C(=O)O)cc3)cc2F)ccc1C1CC1 ; Cc1cc(NC(=O)N2CCC[C@@H]2c2ncc(-c3ccc(C(=O)O)cc3)cc2F)ccc1C1CC1]

4-(6-(1-((4-cyclopropyl-3-methylphenyl)carbamoyl)pyrrolidin-2-yl)-5-fluoropyridin-3-yl)benzoic acid (60 mg) was separated by prep Chiral-HPLC (conditions: Column: CHIRALPAK IK 3*25 cm, 5 μm; Mobile Phase A: Hexane (0.1% FA), Mobile Phase B: EtOH; Flow rate: 40 mL/min; Gradient (B %): isocratic 30% B; Wave Length: 250/286 nm; RT1 (min): 7.06; RT2 (min): 10.32) to give (Compound 66a; Method G, Peak 1, 1.20 min) (S)-4-(6-(1-((4-cyclopropyl-3-methylphenyl)carbamoyl)pyrrolidin-2-yl)-5-fluoropyridin-3-yl)benzoic acid (1^{st} peak, 23.7 mg, 40% yield) as a white solid and (Compound 66b; Method G, Peak 2, 1.85 min) (R)-4-(6-(1-((4-cyclopropyl-3-methylphenyl)carbamoyl)pyrrolidin-2-yl)-5-fluoropyridin-3-yl)benzoic acid (2^{nd} peak, 33.0 mg, 55% yield) as a white solid.

| Cmpd # | Characterization Data |
|---|---|
| 66a | LCMS (ESI) [M + H]⁺: 460.2 |
| | ¹H NMR (400 MHz, DMSO-d₆) δ 8.73 (s, 1H), 8.12-7.99 (m, 4H), 7.89 (d, J = 8.1 Hz, 2H), 7.24 |
| | (s, 1H), 7.15 (dd, J = 8.3, 2.3 Hz, 1H), 6.76 (d, J = 8.4 Hz, 1H), 5.33 (dd, J = 8.1, 4.1 Hz, 1H), 3.80- |
| | 3.71 (m, 1H), 3.61 (q, J = 7.7 Hz, 1H), 2.39-2.22 (m, 4H), 2.17-2.05 (m, 1H), 2.05-1.94 (m, |
| | 1H), 1.94-1.84 (m, 1H), 1.84-1.71 (m, 1H), 0.88-0.76 (m, 2H), 0.54-0.43 (m, 2H). |
| 66b | LCMS (ESI) [M + H]⁺: 460.2 |
| | ¹H NMR (400 MHz, DMSO-d₆) δ 13.13 (bs, 1H), 8.73 (s, 1H), 8.12-7.97 (m, 4H), 7.89 (d, J = 8.4 |
| | Hz, 2H), 7.24 (d, J = 2.3 Hz, 1H), 7.14 (dd, J = 8.3, 2.3 Hz, 1H), 6.75 (d, J = 8.4 Hz, 1H), 5.32 (dt, J = |
| | 5.1, 2.5 Hz, 1H), 3.81-3.68 (m, 1H), 3.67-3.53 (m, 1H), 2.41-2.21 (m, 4H), 2.19-2.05 (m, |
| | 1H), 2.05-1.96 (m, 1H), 1.95-1.84 (m, 1H), 1.84-1.72 (m, 1H), 0.87-0.73 (m, 2H), 0.51-0.39 |
| | (m, 2H). |

### Example 67: (S)-4-(5-fluoro-6-(1-((5-fluoro-6-isopropylpyridin-3-yl)carbamoyl)pyrrolidin-2-yl)pyridin-3-yl)benzoic acid & (R)-4-(5-fluoro-6-(1-((5-fluoro-6-isopropylpyridin-3-yl)carbamoyl)pyrrolidin-2-yl)pyridin-3-yl)benzoic acid

### Step 1: methyl 4-(5-fluoro-6-(1-((5-fluoro-6-isopropylpyridin-3-yl)carbamoyl)pyrrolidin-2-yl)pyridin-3-yl)benzoate

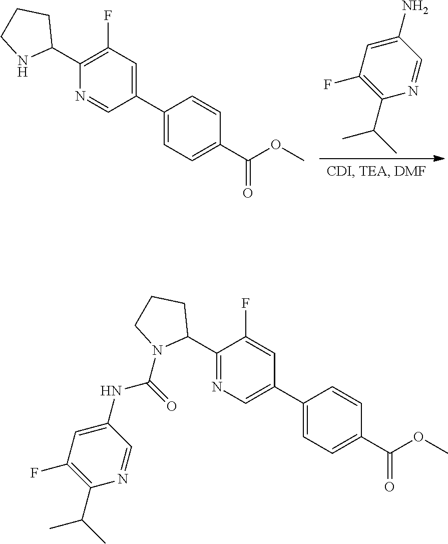

A solution of 5-fluoro-6-isopropylpyridin-3-amine (154 mg, 1.00 mmol), CDI (405 mg, 2.50 mmol) and triethylamine (253 mg, 2.50 mmol) in DMF (2 mL) was stirred at 50° C. for 1 hour. Then, methyl 4-[5-fluoro-6-(pyrrolidin-2-yl)pyridin-3-yl]benzoate (150 mg, 0.499 mmol) was added and the reaction was allowed to stir at 50° C. for an additional 1 hour. Upon completion, the resulting mixture was cooled to room temperature and concentrated under reduced pressure. The crude residue was directly purified by silica gel column chromatography, eluted with PE/EA (1/3), to afford methyl 4-(5-fluoro-6-(1-((5-fluoro-6-isopropylpyridin-3-yl)carbamoyl)pyrrolidin-2-yl)pyridin-3-yl)benzoate (100 mg, 42% yield) as a yellow oil. LCMS (ESI) [M+H]⁺: 481.2

### Step 2: 4-(5-fluoro-6-(1-((5-fluoro-6-isopropylpyridin-3-yl)carbamoyl)pyrrolidin-2-yl)pyridin-3-yl)benzoic acid

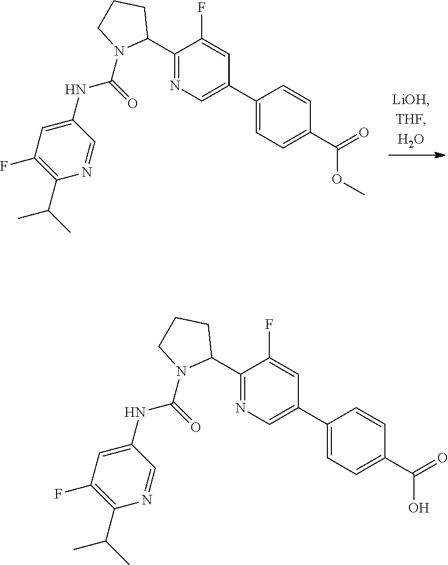 [SMILES: COC(=O)c1ccc(-c2cnc(C3CCCN3C(=O)Nc3cnc(C(C)C)c(F)c3)c(F)c2)cc1 ; CC(C)c1ncc(NC(=O)N2CCCC2c2ncc(-c3ccc(C(=O)O)cc3)cc2F)cc1F]

A solution of methyl 4-(5-fluoro-6-(1-((5-fluoro-6-isopropylpyridin-3-yl)carbamoyl)pyrrolidin-2-yl)pyridin-3-yl)benzoate (100 mg, 0.208 mmol) and LiOH monohydrate (17.6 mg, 0.42 mmol) in H₂O (1 mL) and THF (1 mL) was stirred at 25° C. for 1 hour. Upon completion, the reaction mixture was acidified to pH=2 with HCl (1 M in water). The resulting mixture was extracted with EtOAc (3×10 mL). The combined organic layers were washed with H₂O (10 mL), dried over anhydrous Na₂SO₄, filtered and concentrated under reduced pressure. The crude residue was purified by reversed-phase flash chromatography (conditions: column: C18 silica gel; mobile phase A: Water (0.1% FA), mobile phase B: MeCN; Gradient (B %): 10% to 50% in 20 min; detector: UV 254 nm) to afford 4-(5-fluoro-6-(1-((5-fluoro-6-isopropylpyridin-3-yl)carbamoyl)pyrrolidin-2-yl)pyridin-3-yl)benzoic acid (50 mg, 52% yield) as a white solid. LCMS (ESI) [M+H]⁺: 467.2

### Step 3: (S)-4-(5-fluoro-6-(1-((5-fluoro-6-isopropylpyridin-3-yl)carbamoyl)pyrrolidin-2-yl)pyridin-3-yl)benzoic acid & (R)-4-(5-fluoro-6-(1-((5-fluoro-6-isopropylpyridin-3-yl)carbamoyl)pyrrolidin-2-yl)pyridin-3-yl)benzoic acid

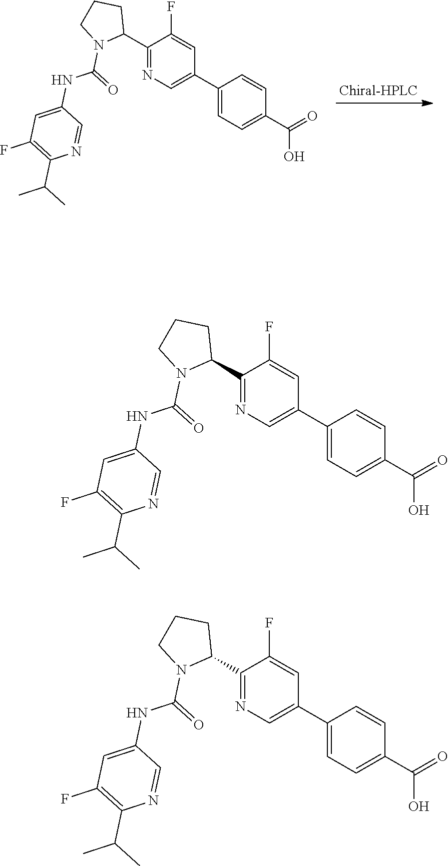 [SMILES: CC(C)c1ncc(NC(=O)N2CCCC2c2ncc(-c3ccc(C(=O)O)cc3)cc2F)cc1F ; CC(C)c1ncc(NC(=O)N2CCC[C@H]2c2ncc(-c3ccc(C(=O)O)cc3)cc2F)cc1F ; CC(C)c1ncc(NC(=O)N2CCC[C@@H]2c2ncc(-c3ccc(C(=O)O)cc3)cc2F)cc1F]

4-(5-fluoro-6-(1-((5-fluoro-6-isopropylpyridin-3-yl)carbamoyl)pyrrolidin-2-yl)pyridin-3-yl)benzoic acid (40 mg, 0.086 mmol) was separated by prep-Chiral-HPLC (conditions: Column: CHIRAL ART Cellulose-SC, 3*25 cm, 5 μm; Mobile Phase A: Hexane (0.1% FA), Mobile Phase B: EtOH; Flow rate: 40 mL/min; Gradient (B %): isocratic 30% B; Wave Length: 244/286 nm; RT1 (min): 6.53; RT2 (min): 7.6) to afford (Compound 67a; Method AA, Peak 1, 0.93 min) (S)-4-(5-fluoro-6-(1-((5-fluoro-6-isopropylpyridin-3-yl)carbamoyl)pyrrolidin-2-yl)pyridin-3-yl)benzoic acid (1^{st} peak, 12.4 mg, 31% yield) and (Compound 67b; Method AA, Peak 2, 1.27 min) (R)-4-(5-fluoro-6-(1-((5-fluoro-6-isopropylpyridin-3-yl)carbamoyl)pyrrolidin-2-yl)pyridin-3-yl)benzoic acid (2^{nd} peak, 11.6 mg, 29% yield).

| Cmpd # | Characterization Data |
|---|---|
| 67a | LCMS (ESI) [M + H]⁺: 467.2 |
| | ¹H NMR (400 MHz, Methanol-d₄) δ 8.64 (s, 1H), 8.32 (d, J = 2.2 Hz, 1H), 8.13 (d, J = 8.4 Hz, 2H), |
| | 7.91 (dd, J = 11.2, 1.9 Hz, 1H), 7.78 (d, J = 8.2 Hz, 2H), 7.73 (dd, J = 12.4, 2.1 Hz, 1H), 5.50-5.40 |
| | (m, 1H), 3.92-3.81 (m, 1H), 3.77-3.63 (m, 1H), 3.28-3.22 (m, 1H), 2.55-2.36 (m, 1H), 2.26- |
| | 1.96 (m, 3H), 1.24 (d, J = 6.9 Hz, 6H). |
| 67b | LCMS (ESI) [M + H]⁺: 467.2 |
| | ¹H NMR (400 MHz, Methanol-d₄) δ 8.63 (d, J = 1.5 Hz, 1H), 8.32 (d, J = 2.1 Hz, 1H), 8.13 (d, J = |
| | 8.4 Hz, 2H), 7.91 (dd, J = 11.1, 1.9 Hz, 1H), 7.78 (d, J = 8.5 Hz, 2H), 7.73 (dd, J = 12.4, 2.1 Hz, |
| | 1H), 5.54-5.42 (m, 1H), 3.93-3.84 (m, 1H), 3.77-3.66 (m, 1H), 3.29-3.23 (m, 1H), 2.52-2.38 |
| | (m, 1H), 2.22-1.98 (m, 3H), 1.24 (d, J = 6.9 Hz, 6H). |

### Example 68: (S)-4-(5-fluoro-6-(1-(3-(4-isopropylphenyl)ureido)-2-methylpropyl)pyridin-3-yl)benzoic acid & (R)-4-(5-fluoro-6-(1-(3-(4-isopropylphenyl)ureido)-2-methylpropyl)pyridin-3-yl)benzoic acid

### Step 1: methyl 4-(5-fluoro-6-formylpyridin-3-yl)benzoate

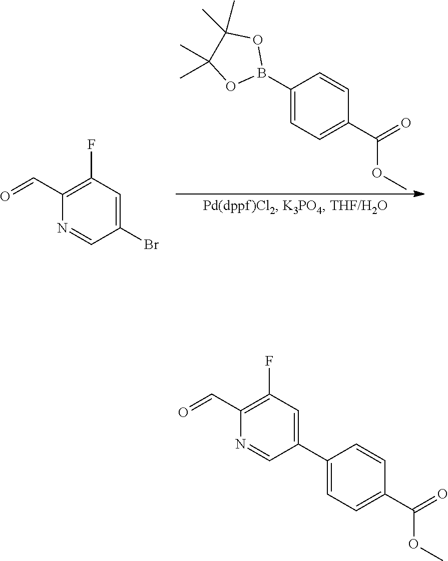

A mixture of 5-bromo-3-fluoropyridine-2-carbaldehyde (1.0 g, 4.90 mmol), methyl 4-(4,4,5,5-tetramethyl-1,3,2-dioxaborolan-2-yl)benzoate (1.28 g, 4.90 mmol), K₃PO₄ (3.12 g, 14.70 mmol) and Pd(dppf)Cl₂ (0.36 g, 0.49 mmol) in THF (10 mL) and H₂O (1 mL) was stirred at 80° C. for 2 hours under nitrogen atmosphere. Upon completion, the resulting mixture was concentrated under reduced pressure. The crude residue was directly purified by silica gel column chromatography, eluted with PE/EA (6:1) to afford methyl 4-(5-fluoro-6-formylpyridin-3-yl)benzoate (800 mg, 63% yield) as an off-white solid. LCMS (ESI) [M+H]⁺: 260.0

### Step 2: methyl 4-(5-fluoro-6-(1-hydroxy-2-methylpropyl)pyridin-3-yl)benzoate

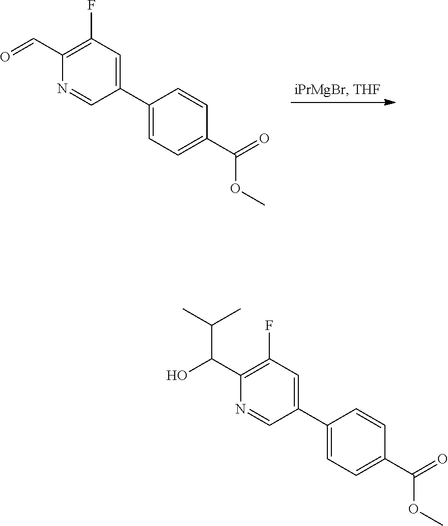 [SMILES: COC(=O)c1ccc(-c2cnc(C=O)c(F)c2)cc1 ; COC(=O)c1ccc(-c2cnc(C(O)C(C)C)c(F)c2)cc1]

i-PrMgBr (3.09 mL, 3.08 mmol, 1 M in THF) was added dropwise into a solution of methyl 4-(5-fluoro-6-formylpyridin-3-yl)benzoate (800 mg, 3.08 mmol) in THF (5 mL) at 0° C. under nitrogen atmosphere. The reaction was allowed to stir at 0° C. for 30 minutes. Upon completion, the reaction was quenched with sat. NH₄Cl (aq.) at 0° C. The aqueous layer was extracted with EtOAc (5×5 mL). The combined organic layers were concentrated under reduced pressure. The crude residue was purified by silica gel column chromatography, eluted with PE/EA (5:1) to afford methyl 4-(5-fluoro-6-(1-hydroxy-2-methylpropyl)pyridin-3-yl)benzoate (600 mg, 64% yield) as a white solid. LCMS (ESI) [M+H]⁺: 304.1

### Step 3: methyl 4-(5-fluoro-6-isobutyrylpyridin-3-yl)benzoate

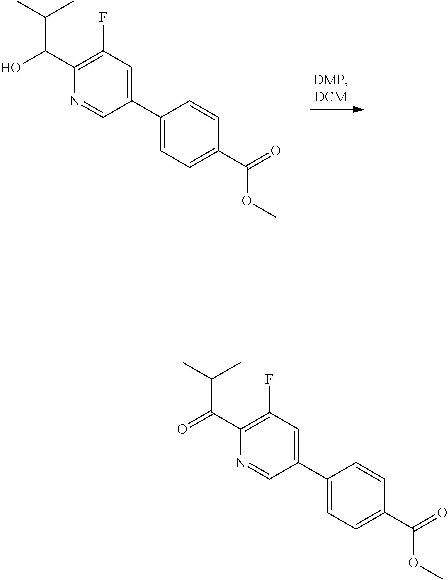

A mixture of methyl 4-[5-fluoro-6-(1-hydroxy-2-methylpropyl)pyridin-3-yl]benzoate (600 mg, 1.97 mmol) and Dess-Martin periodinane (838 mg, 1.97 mmol) in DCM (5 mL) was stirred at 25° C. for 3 hours. Upon completion, the reaction mixture was poured into water (20 mL). The aqueous layer was extracted with EtOAc (4×5 mL). The combined organic layers were concentrated under reduced pressure. The crude residue was purified by silica gel column chromatography, eluted with PE/EA (8:1) to afford methyl 4-(5-fluoro-6-isobutyrylpyridin-3-yl)benzoate (420 mg, 70% yield) as a white solid. LCMS (ESI) [M+H]⁺: 302.1

### Step 4: methyl 4-(6-(1-amino-2-methylpropyl)-5-fluoropyridin-3-yl)benzoate

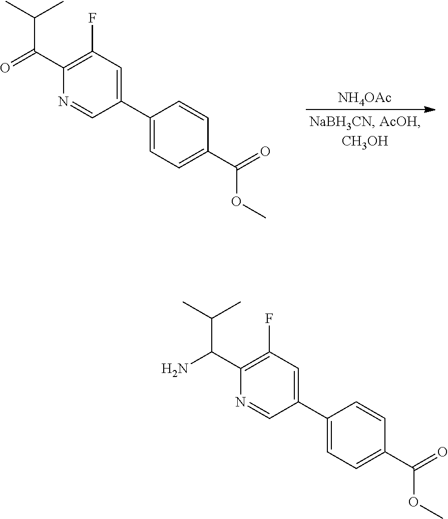 [SMILES: COC(=O)c1ccc(-c2cnc(C(=O)C(C)C)c(F)c2)cc1 ; COC(=O)c1ccc(-c2cnc(C(N)C(C)C)c(F)c2)cc1 ; "NH4OAc"]

A mixture of methyl 4-[5-fluoro-6-(2-methylpropanoyl)pyridin-3-yl]benzoate (500 mg, 1.65 mmol), ammonium acetate (639 mg, 8.29 mmol), sodium cyanoborohydride (125 mg, 1.99 mmol) and acetic acid (99 mg, 1.65 mmol) in methanol (5 mL) was stirred at 60° C. overnight. Upon completion, the resulting mixture was concentrated under reduced pressure. The crude residue was directly purified by silica gel column chromatography, eluted with DCM/MeOH (10:1) to afford methyl 4-(6-(1-amino-2-methylpropyl)-5-fluoropyridin-3-yl)benzoate (300 mg, 60% yield) as a white solid. LCMS (ESI) [M+H]⁺: 303.1

### Step 5: methyl 4-(5-fluoro-6-(1-(3-(4-isopropylphenyl)ureido)-2-methylpropyl)pyridin-3-yl)benzoate

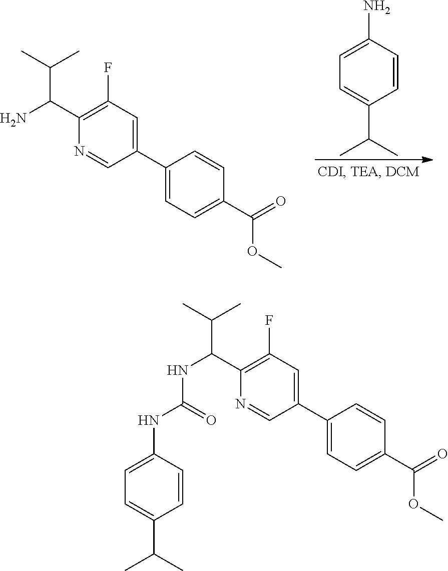

A mixture of methyl 4-(6-(1-amino-2-methylpropyl)-5-fluoropyridin-3-yl)benzoate (300 mg, 0.99 mmol), 4-isopropylaniline (134 mg, 0.99 mmol), triethylamine (202 mg, 2.00 mmol) and CDI (482 mg, 2.98 mmol) in DCM (3 mL) was stirred at 25° C. overnight. Upon completion, the resulting mixture was concentrated under reduced pressure. The crude residue was purified by silica gel column chromatography, eluted with PE/EA (1:1) to afford methyl 4-(5-fluoro-6-(1-(3-(4-isopropylphenyl)ureido)-2-methylpropyl)pyridin-3-yl)benzoate (160 mg, 35% yield) as a white solid. LCMS (ESI) [M+H]⁺: 463.2

### Step 6: 4-(5-fluoro-6-(1-(3-(4-isopropylphenyl)ureido)-2-methylpropyl)pyridin-3-yl)benzoic acid

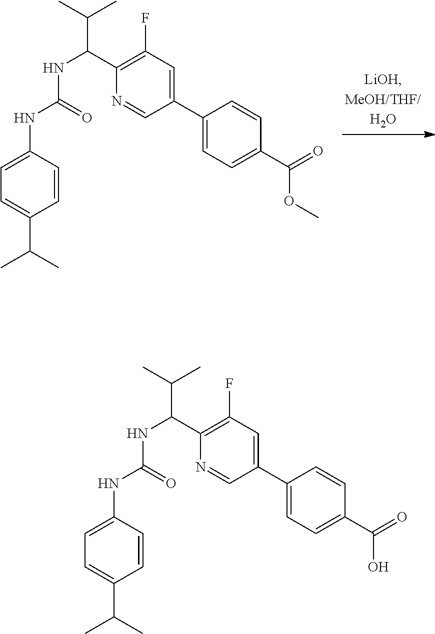 [SMILES: COC(=O)c1ccc(-c2cnc(C(NC(=O)Nc3ccc(C(C)C)cc3)C(C)C)c(F)c2)cc1 ; CC(C)c1ccc(NC(=O)NC(c2ncc(-c3ccc(C(=O)O)cc3)cc2F)C(C)C)cc1]

A mixture of methyl 4-(5-fluoro-6-(1-(3-(4-isopropylphenyl)ureido)-2-methylpropyl)pyridin-3-yl)benzoate (160 mg, 0.34 mmol) and LiOH monohydrate (42 mg, 1.03 mmol) in THF (1 mL), methanol (1 mL) and H₂O (1 mL) was stirred at 25° C. overnight. Upon completion, the resulting mixture was concentrated under reduced pressure. The crude residue was directly purified by reversed-phase flash chromatography (conditions: column: C18 silica gel; mobile phase A: Water (0.1% FA), mobile phase B: MeCN; Gradient (B %): 20% to 60% in 10 min; detector: UV 254 nm) to afford 4-(5-fluoro-6-(1-(3-(4-isopropylphenyl)ureido)-2-methylpropyl)pyridin-3-yl)benzoic acid (120 mg, 77% yield) as a white solid. LCMS (ESI) [M+H]⁺: 450.2

### Step 7: (S)-4-(5-fluoro-6-(1-(3-(4-isopropylphenyl)ureido)-2-methylpropyl)pyridin-3-yl)benzoic acid & (R)-4-(5-fluoro-6-(1-(3-(4-isopropylphenyl)ureido)-2-methylpropyl)pyridin-3-yl)benzoic acid

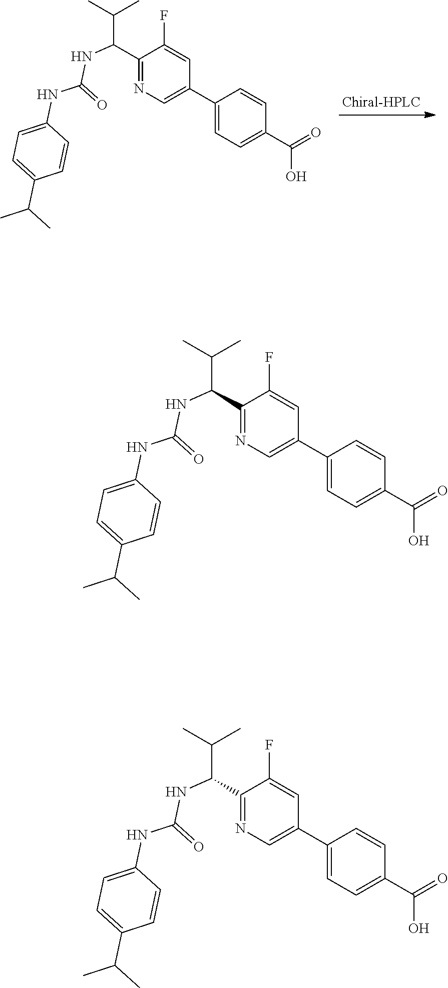 [SMILES: CC(C)c1ccc(NC(=O)NC(c2ncc(-c3ccc(C(=O)O)cc3)cc2F)C(C)C)cc1 ; CC(C)c1ccc(NC(=O)N[C@H](c2ncc(-c3ccc(C(=O)O)cc3)cc2F)C(C)C)cc1 ; CC(C)c1ccc(NC(=O)N[C@@H](c2ncc(-c3ccc(C(=O)O)cc3)cc2F)C(C)C)cc1]

4-(5-fluoro-6-(1-(3-(4-isopropylphenyl)ureido)-2-methylpropyl)pyridin-3-yl)benzoic acid (90 mg) was separated by Prep-CHIRAL-HPLC (conditions: Column: CHIRALPAK IG, 3*25 cm, 5 μm; Mobile Phase A: Hexane (0.1% TFA), Mobile Phase B: EtOH; Flow rate: 40 mL/min; Gradient (B %): isocratic 50% B; Wave Length: 240/250 nm; RT1 (min): 5.1; RT2 (min): 11.2) to afford (Compound 68a; Method AB, Peak 1, 0.66 min) (S)-4-(5-fluoro-6-(1-(3-(4-isopropylphenyl)ureido)-2-methylpropyl)pyridin-3-yl)benzoic acid (1^{st} peak, 31.7 mg, 42% yield) and (Compound 68b; Method AB, Peak 2, 1.16 min) (R)-4-(5-fluoro-6-(1-(3-(4-isopropylphenyl)ureido)-2-methylpropyl)pyridin-3-yl)benzoic acid (2^{nd} peak, 34.5 mg, 46% yield) as a white solid.

| Cmpd # | Characterization Data |
|---|---|
| 68a | LCMS (ESI) [M + H]⁺: 450.2 |
| | ¹H NMR (400 MHz, Methanol-d₄) δ 8.75 (s, 1H), 8.14 (d, J = 8.4 Hz, 2H), 7.94 (dd, J = 10.7, 1.9 Hz, |
| | 1H), 7.82 (d, J = 8.4 Hz, 2H), 7.24 (d, J = 8.5 Hz, 2H), 7.10 (d, J = 8.6 Hz, 2H), 5.17-5.07 (m, 1H), |
| | 2.82 (hept, J = 6.9 Hz, 1H), 2.16 (hept, J = 6.9 Hz, 1H), 1.20 (d, J = 6.9 Hz, 6H), 1.05 (d, J = 6.8 Hz, |
| | 3H), 0.94 (d, J = 6.7 Hz, 3H). |
| 68b | LCMS (ESI) [M + H]⁺: 450.2 |
| | ¹H NMR (400 MHz, Methanol-d₄) δ 8.75 (d, J = 1.7 Hz, 1H), 8.14 (d, J = 8.5 Hz, 2H), 7.94 (dd, J = |
| | 10.7, 1.9 Hz, 1H), 7.82 (d, J = 8.4 Hz, 2H), 7.24 (d, J = 8.6 Hz, 2H), 7.10 (d, J = 8.5 Hz, 2H), 5.13 |
| | (dd, J = 7.3, 1.3 Hz, 1H), 2.82 (p, J = 6.9 Hz, 1H), 2.16 (q, J = 6.8 Hz, 1H), 1.20 (d, J = 6.9 Hz, 6H), |
| | 1.05 (d, J = 6.7 Hz, 3H), 0.94 (d, J = 6.7 Hz, 3H). |

### Example 69: (S)-3-(5-fluoro-6-(1-((4-isopropylphenyl)carbamoyl)pyrrolidin-2-yl)pyridin-3-yl)benzoic acid & (R)-3-(5-fluoro-6-(1-((4-isopropylphenyl)carbamoyl)pyrrolidin-2-yl)pyridin-3-yl)benzoic acid

### Step 1: methyl 3-(6-chloro-5-fluoropyridin-3-yl)benzoate

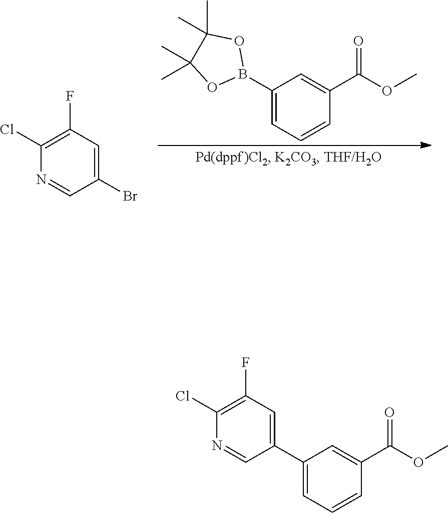

A mixture of 5-bromo-2-chloro-3-fluoropyridine (2.0 g, 9.50 mmol), methyl 3-(4,4,5,5-tetramethyl-1,3,2-dioxaborolan-2-yl)benzoate (2.49 g, 9.50 mmol), Pd(dppf)Cl₂ (800 mg, 1.09 mmol) and K₂CO₃ (4.0 g, 28.9 mmol) in THF (20 mL) and H₂O (4 mL) was stirred at 80° C. for 1 hour under nitrogen atmosphere. Upon completion, the resulting mixture was diluted with H₂O (20 mL). The resulting mixture was extracted with ethyl acetate (3×80 mL). The combined organic layers were washed with H₂O (100 mL), dried over anhydrous Na₂SO₄, filtered and concentrated under reduced pressure. The crude residue was purified by silica gel column chromatography, eluted with petroleum ether/ethyl acetate (10:1) to afford methyl 3-(6-chloro-5-fluoropyridin-3-yl)benzoate (700 mg, 28% yield) as a white solid.

### Step 2: tert-butyl 2-(3-fluoro-5-(3-(methoxycarbonyl)phenyl)pyridin-2-yl)pyrrolidine-1-carboxylate

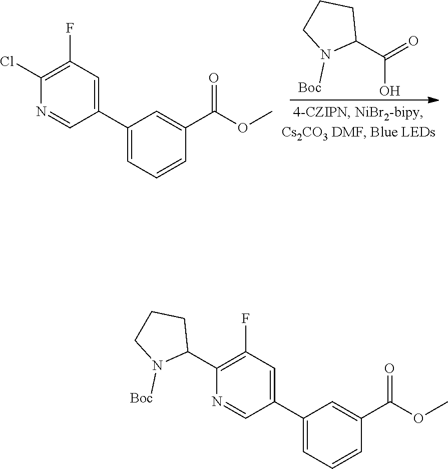 [SMILES: COC(=O)c1cccc(-c2cnc(Cl)c(F)c2)c1 ; CC(C)(C)OC(=O)N1CCCC1C(=O)O ; COC(=O)c1cccc(-c2cnc(C3CCCN3C(=O)OC(C)(C)C)c(F)c2)c1]

A solution of methyl 3-(6-chloro-5-fluoropyridin-3-yl)benzoate (600 mg, 2.25 mmol), Cs₂CO₃ (2.2 g, 6.77 mmol,), tetrakis(9H-carbazol-9-yl)benzene-1,4-dicarbonitrile (178 mg, 0.22 mmol) and NiBr₂·bipy (423 mg, 1.12 mmol) in DMF (24 mL) was treated with 1-(tert-butoxycarbonyl)pyrrolidine-2-carboxylic acid (481 mg, 2.23 mmol) at room temperature under nitrogen atmosphere. The resulting mixture was stirred under irradiation at 450 nm—with blue LEDs for 24 hours. Upon completion, the resulting mixture was diluted with H₂O (20 mL) and extracted with ethyl acetate (3×100 mL). The combined organic layers were washed with H₂O (3×50 mL), dried over anhydrous Na₂SO₄, filtered and concentrated under reduced pressure. The crude residue was purified by silica gel column chromatography, eluted with petroleum ether/ethyl acetate (3:1) to afford tert-butyl 2-(3-fluoro-5-(3-(methoxycarbonyl)phenyl)pyridin-2-yl)pyrrolidine-1-carboxylate (150 mg, 17% yield) as a brown oil. LCMS (ESI) [M+H]⁺: 401

### Step 3: methyl 3-(5-fluoro-6-(pyrrolidin-2-yl)pyridin-3-yl)benzoate

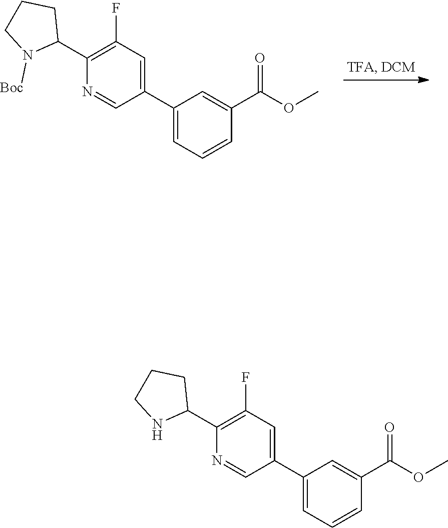 [SMILES: COC(=O)c1cccc(-c2cnc(C3CCCN3C(=O)OC(C)(C)C)c(F)c2)c1 ; COC(=O)c1cccc(-c2cnc(C3CCCN3)c(F)c2)c1]

A solution of tert-butyl 2-(3-fluoro-5-(3-(methoxycarbonyl)phenyl)pyridin-2-yl)pyrrolidine-1-carboxylate (150 mg, 0.37 mmol) and TFA (1 mL) in DCM (3 mL) was stirred at room temperature for 1 hour. Upon completion, the resulting mixture was concentrated under reduced pressure to afford methyl 3-(5-fluoro-6-(pyrrolidin-2-yl)pyridin-3-yl)benzoate (300 mg, crude) as a yellow oil, which was used directly in the next step without further purification. LCMS (ESI) [M+H]⁺: 301.

### Step 4: methyl 3-(5-fluoro-6-(1-((4-isopropylphenyl)carbamoyl)pyrrolidin-2-yl)pyridin-3-yl)benzoate

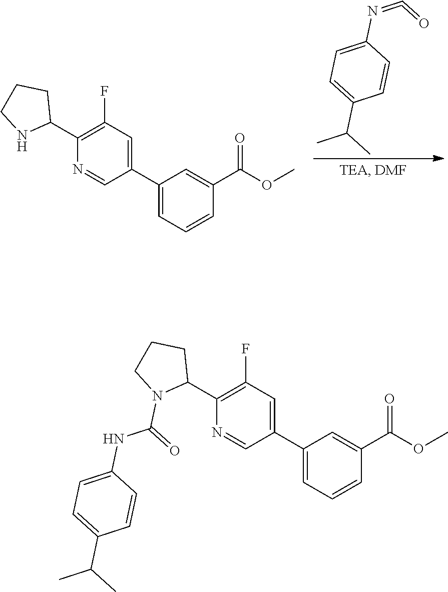

A solution of methyl 3-(5-fluoro-6-(pyrrolidin-2-yl)pyridin-3-yl)benzoate (200 mg, 0.66 mmol), TEA (202 mg, 1.99 mmol) and 1-isocyanato-4-isopropylbenzene (107 mg, 0.66 mmol) in DMF (3 mL) was stirred at room temperature for 1 hour. The resulting mixture was diluted with H₂O (20 mL) and extracted with ethyl acetate (3×50 mL). The combined organic layers were washed with H₂O (3×50 mL), dried over anhydrous Na₂SO₄, filtered and concentrated under reduced pressure. The crude residue was purified by silica gel column chromatography, eluted with petroleum ether/ethyl acetate (3:1) to afford methyl 3-(5-fluoro-6-(1-((4-isopropylphenyl)carbamoyl)pyrrolidin-2-yl)pyridin-3-yl)benzoate (130 mg, 42% yield) as a yellow oil. LCMS (ESI) [M+H]⁺: 461

### Step 5: 3-(5-fluoro-6-(1-((4-isopropylphenyl)carbamoyl)pyrrolidin-2-yl)pyridin-3-yl)benzoic acid

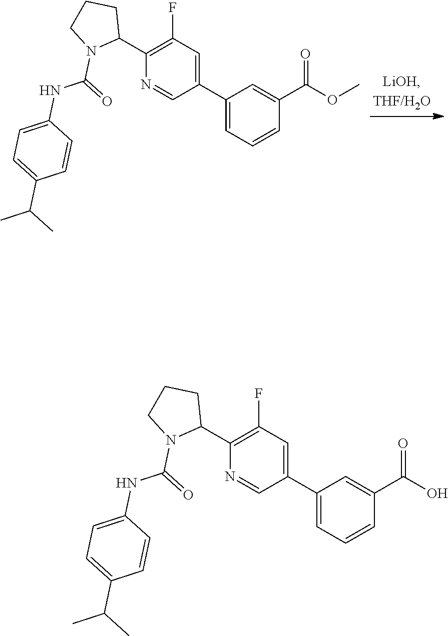

A mixture of methyl 3-(5-fluoro-6-(1-((4-isopropylphenyl)carbamoyl)pyrrolidin-2-yl)pyridin-3-yl)benzoate (130 mg, 0.28 mmol) and LiOH monohydrate (47 mg, 1.12 mmol) in H₂O (0.3 mL) and THF (3 mL) was stirred at room temperature for 1 hour. Upon completion, the resulting mixture concentrated under reduced pressure. The crude residue was purified by reversed phase flash chromatography (conditions: column: C18 silica gel; mobile phase A: Water (0.1% FA), mobile phase B: MeCN; Gradient (B %): 0 to 100% in 25 min; detector: UV 254 nm) to afford 3-(5-fluoro-6-(1-((4-isopropylphenyl)carbamoyl)pyrrolidin-2-yl)pyridin-3-yl)benzoic acid (150 mg, 79% yield) as a white solid. LCMS (ESI) [M+H]⁺: 448.2

### Step 6: (S)-3-(5-fluoro-6-(1-((4-isopropylphenyl)carbamoyl)pyrrolidin-2-yl)pyridin-3-yl)benzoic acid & (R)-3-(5-fluoro-6-(1-((4-isopropylphenyl)carbamoyl)pyrrolidin-2-yl)pyridin-3-yl)benzoic acid

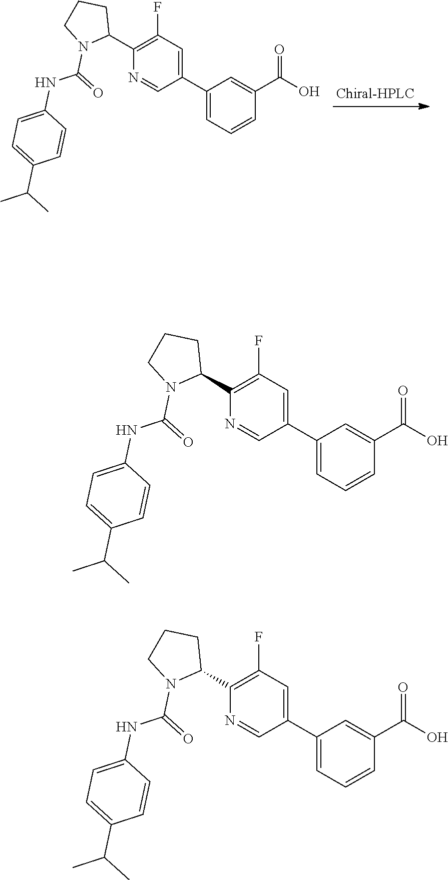 [SMILES: CC(C)c1ccc(NC(=O)N2CCCC2c2ncc(-c3cccc(C(=O)O)c3)cc2F)cc1 ; CC(C)c1ccc(NC(=O)N2CCC[C@H]2c2ncc(-c3cccc(C(=O)O)c3)cc2F)cc1 ; CC(C)c1ccc(NC(=O)N2CCC[C@@H]2c2ncc(-c3cccc(C(=O)O)c3)cc2F)cc1]

3-(5-fluoro-6-(1-((4-isopropylphenyl)carbamoyl)pyrrolidin-2-yl)pyridin-3-yl)benzoic acid (150 mg, 0.20 mmol) was separated by prep Chiral-HPLC (conditions: Column: CHIRALPAK IE, 3*25 cm, 5 μm; Mobile Phase A: MtBE (0.1% FA), Mobile Phase B: MeOH; Flow rate: 40 mL/min; Gradient (B %): isocratic 10% B; Wave Length: 246/206 nm) to afford (Compound 69a; Method AC, Peak 1, 1.41 min) (S)-3-(5-fluoro-6-(1-((4-isopropylphenyl)carbamoyl)pyrrolidin-2-yl)pyridin-3-yl)benzoic acid (1^{st} peak, 50.9 mg, 57% yield) and (Compound 69b; Method AC, Peak 2, 1.67 min) (R)-3-(5-fluoro-6-(1-((4-isopropylphenyl)carbamoyl)pyrrolidin-2-yl)pyridin-3-yl)benzoic acid (2^{nd} peak, 54.5 mg, 61% yield) as a white solid.

| Cmpd # | Characterization Data |
|---|---|
| 69a | LCMS (ESI) [M + H]⁺: 448.2 |
| | ¹H NMR (400 MHz, DMSO-d₆) δ 13.15 (bs, 1H), 8.68 (s, 1H), 8.22 (s, 1H), 8.14 (s, 1H), 8.09- |
| | 7.93 (m, 3H), 7.62 (t, J = 7.7 Hz, 1H), 7.33 (d, J = 8.6 Hz, 2H), 7.04 (d, J = 8.6 Hz, 2H), 5.38-5.27 |
| | (m, 1H), 3.81-3.69 (m, 1H), 3.67-3.56 (m, 1H), 2.78 (hept, J = 7.0 Hz, 1H), 2.36-2.31 (m, 1H), |
| | 2.15-2.07 (m, 1H), 2.04-1.96 (m, 1H), 1.92-1.83 (m, 1H), 1.14 (d, J = 6.9 Hz, 6H). |
| 69b | LCMS (ESI) [M + H]⁺: 448.2 |
| | ¹H NMR (400 MHz, DMSO-d₆) δ 8.68 (s, 1H), 8.22 (s, 1H), 8.14 (s, 1H), 8.06-7.93 (m, 3H), 7.62 |
| | (t, J = 7.7 Hz, 1H), 7.33 (d, J = 8.6 Hz, 2H), 7.04 (d, J = 8.6 Hz, 2H), 5.34 (dd, J = 8.4, 4.1 Hz, 1H), |
| | 3.81-3.70 (m, 1H), 3.66-3.57 (m, 1H), 2.78 (hept, J = 6.8 Hz, 1H), 2.37-2.30 (m, 1H), 2.16- |
| | 2.07 (m, 1H), 2.07-1.96 (m, 1H), 1.94-1.83 (m, 1H), 1.14 (d, J = 6.8 Hz, 6H). |

### Example 70: (S)-4-(5-fluoro-6-(1-((4-methyl-5-(trifluoromethyl)pyridin-2-yl)carbamoyl)pyrrolidin-2-yl)pyridin-3-yl)benzoic acid & (R)-4-(5-fluoro-6-(1-((4-methyl-5-(trifluoromethyl)pyridin-2-yl)carbamoyl)pyrrolidin-2-yl)pyridin-3-yl)benzoic acid

### Step 1: methyl 4-(5-fluoro-6-(1-((4-methyl-5-(trifluoromethyl)pyridin-2-yl)carbamoyl)pyrrolidin-2-yl)pyridin-3-yl)benzoate

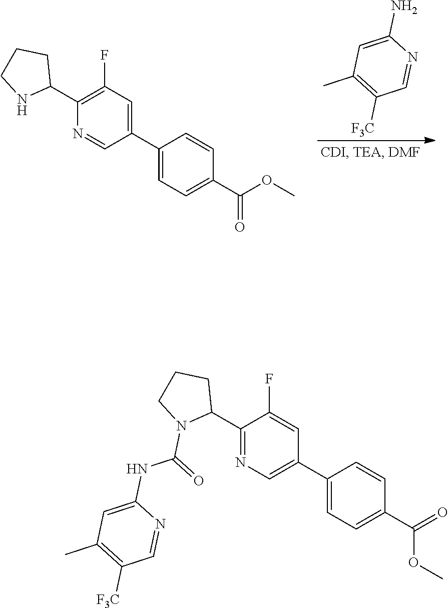

A mixture of 4-methyl-5-(trifluoromethyl)pyridin-2-amine (300 mg, 1.70 mmol), triethylamine (861 mg, 8.51 mmol) and CDI (1.1 g, 6.81 mmol) in DMF (3 mL) was stirred at 50° C. for 30 minutes. Then, methyl 4-[5-fluoro-6-(pyrrolidin-2-yl)pyridin-3-yl]benzoate (358 mg, 1.19 mmol) was added and the resulting mixture was allowed to stir at 50° C. for an additional 30 minutes. Upon completion, the mixture was concentrated under reduced pressure. The crude residue was purified by reversed phase flash chromatography (conditions: column: C18 silica gel; mobile phase A: Water (0.1% NH₄HCO₃), mobile phase B: MeCN; Gradient (B %): 0 to 100% in 25 min; detector: UV 254 nm) to afford methyl 4-(5-fluoro-6-(1-((4-methyl-5-(trifluoromethyl)pyridin-2-yl)carbamoyl)pyrrolidin-2-yl)pyridin-3-yl)benzoate (200 mg, 23% yield) as a brown solid. LCMS (ESI) [M+H]⁺: 503

### Step 2: 4-(5-fluoro-6-(1-((4-methyl-5-(trifluoromethyl)pyridin-2-yl)carbamoyl)pyrrolidin-2-yl)pyridin-3-yl)benzoic acid

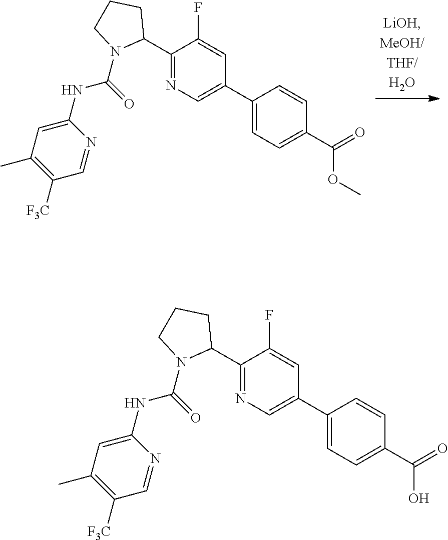

A solution of methyl 4-(5-fluoro-6-(1-((4-methyl-5-(trifluoromethyl)pyridin-2-yl)carbamoyl)pyrrolidin-2-yl)pyridin-3-yl)benzoate (200 mg, 0.39 mmol) and LiOH monohydrate (66.8 mg, 1.59 mmol) in THF (2 mL) and H₂O (0.3 mL) was stirred at 60° C. for 2 hours. Upon completion, the resulting mixture was concentrated under reduced pressure. The crude residue was purified by reversed phase flash chromatography (conditions: column: C18 silica gel; mobile phase A: Water (0.1% FA), mobile phase B: MeCN; Gradient (B %): 0 to 100% in 25 min; detector, UV 254 nm) to afford 4-(5-fluoro-6-(1-((4-methyl-5-(trifluoromethyl)pyridin-2-yl)carbamoyl)pyrrolidin-2-yl)pyridin-3-yl)benzoic acid (95 mg, 49% yield) as a white solid. LCMS (ESI) [M+H]⁺: 489.2

### Step 3: (S)-4-(5-fluoro-6-(1-((4-methyl-5-(trifluoromethyl)pyridin-2-yl)carbamoyl)pyrrolidin-2-yl)pyridin-3-yl)benzoic acid & (R)-4-(5-fluoro-6-(1-((4-methyl-5-(trifluoromethyl)pyridin-2-yl)carbamoyl)pyrrolidin-2-yl)pyridin-3-yl)benzoic acid

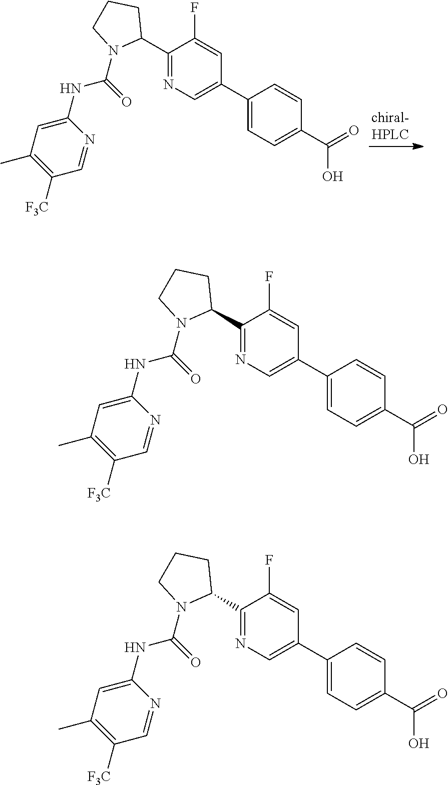 [SMILES: Cc1cc(NC(=O)N2CCCC2c2ncc(-c3ccc(C(=O)O)cc3)cc2F)ncc1C(F)(F)F ; Cc1cc(NC(=O)N2CCC[C@H]2c2ncc(-c3ccc(C(=O)O)cc3)cc2F)ncc1C(F)(F)F ; Cc1cc(NC(=O)N2CCC[C@@H]2c2ncc(-c3ccc(C(=O)O)cc3)cc2F)ncc1C(F)(F)F]

4-(5-fluoro-6-(1-((4-methyl-5-(trifluoromethyl)pyridin-2-yl)carbamoyl)pyrrolidin-2-yl)pyridin-3-yl)benzoic acid (95 mg, 0.19 mmol) was separated by prep Chiral-HPLC (conditions: Column: CHIRAL ART Cellulose-SB, 3*25 cm, 5 μm; Mobile Phase A: Hexane (0.1% FA), Mobile Phase B: EtOH; Flow rate: 40 mL/min; Gradient (B %): isocratic 20% B; Wave Length: 248/286 nm; RT1 (min): 9.33; RT2 (min): 11.25) to afford (Compound 70a; Method L; Peak 1, 1.02 min) (S)-4-(5-fluoro-6-(1-((4-methyl-5-(trifluoromethyl)pyridin-2-yl)carbamoyl)pyrrolidin-2-yl)pyridin-3-yl)benzoic acid (1^{st} peak, 35.8 mg, 38% yield) and (Compound 70b; Method L; Peak 2, 1.20 min) (R)-4-(5-fluoro-6-(1-((4-methyl-5-(trifluoromethyl)pyridin-2-yl)carbamoyl)pyrrolidin-2-yl)pyridin-3-yl)benzoic acid (2^{nd} peak, 34.3 mg, 36% yield) as a white solid.

| Cmpd # | Characterization Data |
|---|---|
| 70a | LCMS (ESI) [M + H]⁺: 489.1 |
| | ¹H NMR (400 MHz, DMSO-d₆) δ 9.29 (bs, 1H), 8.74 (s, 1H), 8.43 (s, 1H), 8.08 (d, J = 11.8 Hz, |
| | 1H), 8.02 (d, J = 8.3 Hz, 2H), 7.87 (d, J = 8.5 Hz, 3H), 5.46-5.29 (m, 1H), 3.88-3.79 (m, 1H), |
| | 3.75-3.63 (m, 1H), 2.40-2.31 (m, 4H), 2.07-1.85 (m, 3H). |
| 70b | LCMS (ESI) [M + H]⁺: 489.1 |
| | ¹H NMR (400 MHz, DMSO-d₆) δ 9.30 (bs, 1H), 8.74 (s, 1H), 8.43 (s, 1H), 8.09 (d, J = 11.0 Hz, |
| | 1H), 8.02 (d, J = 8.1 Hz, 2H), 7.87 (d, J = 11.1 Hz, 3H), 5.46-5.31 (m, 1H), 3.90-3.77 (m, 1H), |
| | 3.76-3.63 (m, 1H), 2.39-2.31 (m, 4H), 2.05-1.85 (m, 3H). |

### Example 71: (S)-4-(5-fluoro-6-(1-((4-isopropylphenyl)carbamoyl)piperidin-2-yl)pyridin-3-yl)benzoic acid & (R)-4-(5-fluoro-6-(1-((4-isopropylphenyl)carbamoyl)piperidin-2-yl)pyridin-3-yl)benzoic acid

### Step 1: methyl 4-(6-chloro-5-fluoropyridin-3-yl)benzoate

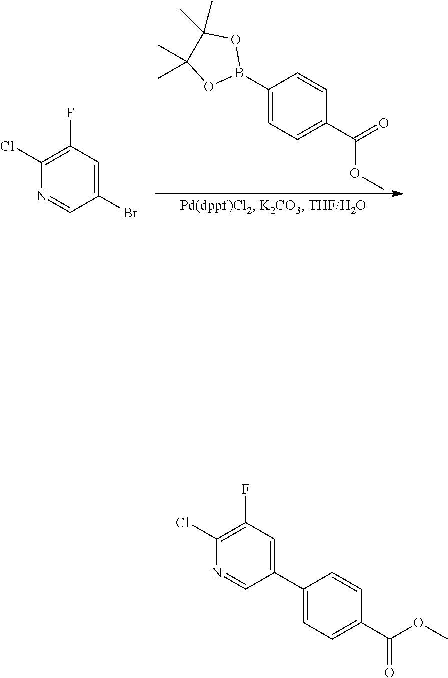

A solution of K₂CO₃ (29.5 g, 214 mmol) in H₂O (30 mL) was added to a mixture of 5-bromo-2-chloro-3-fluoropyridine (15.0 g, 71.3 mmol), methyl 3-(4,4,5,5-tetramethyl-1,3,2-dioxaborolan-2-yl)benzoate (28.0 g, 107 mmol) and Pd(dppf)Cl₂ (2.6 g, 3.56 mmol) in THF (200 mL) under nitrogen atmosphere. The mixture was stirred at 80° C. for 2 hours. Upon completion, the resulting mixture was extracted with ethyl acetate (2×100 mL). The combined organic layers were washed with H₂O (200 mL), dried over anhydrous Na₂SO₄, filtered and concentrated under reduced pressure. The crude residue was purified by silica gel column chromatography, eluted with petroleum ether/ethyl acetate (10:1) to afford methyl 4-(6-chloro-5-fluoropyridin-3-yl)benzoate (11.0 g, 58% yield) as a white solid. LCMS (ESI) [M+H]⁺: 266

### Step 2: tert-butyl 2-(3-fluoro-5-(4-(methoxycarbonyl)phenyl)pyridin-2-yl)piperidine-1-carboxylate

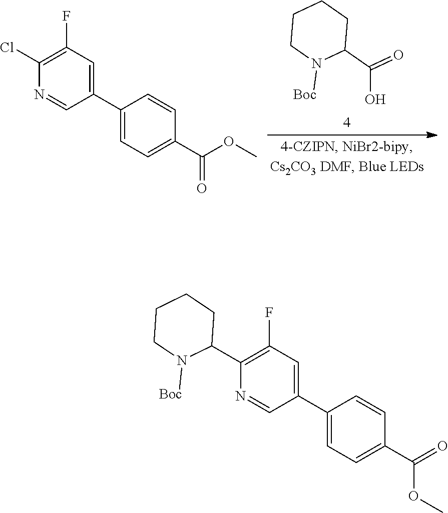 [SMILES: COC(=O)c1ccc(-c2cnc(Cl)c(F)c2)cc1 ; CC(C)(C)OC(=O)N1CCCCC1C(=O)O ; COC(=O)c1ccc(-c2cnc(C3CCCCN3C(=O)OC(C)(C)C)c(F)c2)cc1]

A solution of methyl 4-(6-chloro-5-fluoropyridin-3-yl)benzoate (2.0 g, 7.52 mmol), Cs₂CO₃ (7.3 g, 22.6 mmol), tetrakis(9H-carbazol-9-yl)benzene-1,4-dicarbonitrile (0.3 g, 0.37 mmol) and NiBr₂·bipy (0.3 g, 0.75 mmol) in DMF (120 mL) was treated with 1-(tert-butoxycarbonyl)piperidine-2-carboxylic acid (1.7 g, 7.52 mmol) at room temperature under nitrogen atmosphere. The resulting mixture was stirred under irradiation at 450 nm using blue LEDs for 24 hours. Upon completion, the resulting mixture was diluted with H₂O (100 ml) and extracted with ethyl acetate (3×100 mL). The combined organic layers were washed with H₂O (3×100 mL), dried over anhydrous Na₂SO₄, filtered and concentrated under reduced pressure. The crude residue was purified by silica gel column chromatography, eluted with petroleum ether/ethyl acetate (3:1) to afford tert-butyl 2-(3-fluoro-5-(4-(methoxycarbonyl)phenyl)pyridin-2-yl)piperidine-1-carboxylate (200 mg, 6.4% yield) as a brown oil. LCMS (ESI) [M+H]⁺: 415

### Step 3: methyl 4-(5-fluoro-6-(piperidin-2-yl)pyridin-3-yl)benzoate

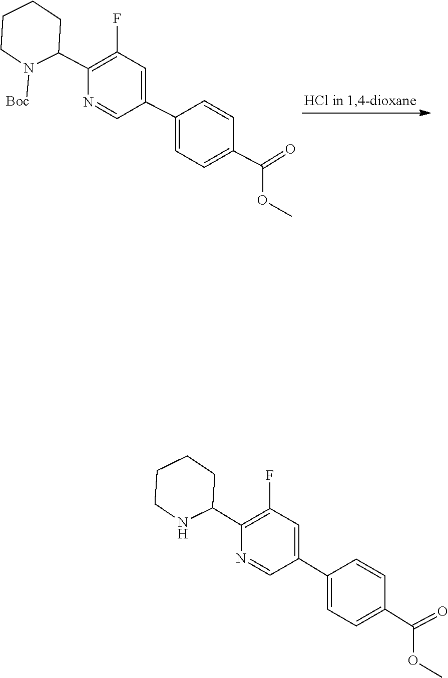 [SMILES: COC(=O)c1ccc(-c2cnc(C3CCCCN3C(=O)OC(C)(C)C)c(F)c2)cc1 ; COC(=O)c1ccc(-c2cnc(C3CCCCN3)c(F)c2)cc1]

To a mixture of tert-butyl 2-(3-fluoro-5-(4-(methoxycarbonyl)phenyl)pyridin-2-yl)piperidine-1-carboxylate (150 mg, 0.36 mmol) in dioxane (3 mL) was added 4 M HCl in 1,4-dioxane (3 mL, 12 mmol) at room temperature. The resulting mixture was stirred for 1 hour. Upon completion, the resulting mixture was concentrated under reduced pressure to afford methyl 4-(5-fluoro-6-(piperidin-2-yl)pyridin-3-yl)benzoate (158 mg, crude) as a yellow solid, which was used directly in the next step without further purification. LCMS (ESI) [M+H]⁺: 315

### Step 4: methyl 4-(5-fluoro-6-(1-((4-isopropylphenyl)carbamoyl)piperidin-2-yl)pyridin-3-yl)benzoate

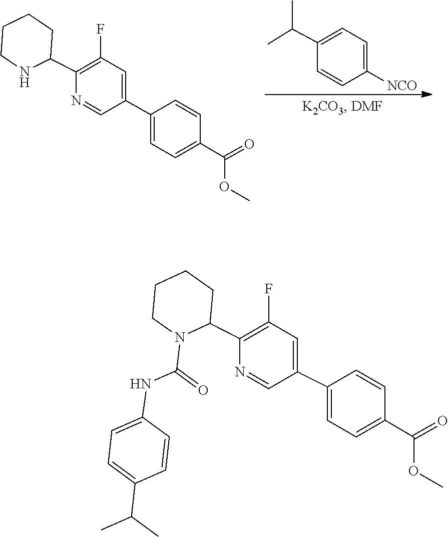

A solution of methyl 4-(5-fluoro-6-(piperidin-2-yl)pyridin-3-yl)benzoate (160 mg, 0.50 mmol), 1-isocyanato-4-isopropylbenzene (82 mg, 0.50 mmol) and K₂CO₃ (281 mg, 2.03 mmol) in DMF (2 mL) was stirred at room temperature for 30 minutes. Upon completion, the resulting mixture was diluted with H₂O (20 mL). The resulting mixture was extracted with ethyl acetate (3×30 mL). The combined organic layers were washed with H₂O (3×30 mL), dried over anhydrous Na₂SO₄, filtered and concentrated under reduced pressure. The crude residue was purified by silica gel column chromatography, eluted with petroleum ether/ethyl acetate (3:1) to afford methyl 4-(5-fluoro-6-(1-((4-isopropylphenyl)carbamoyl)piperidin-2-yl)pyridin-3-yl)benzoate (160 mg, 66% yield) as a colorless oil. LCMS (ESI) [M+H]⁺: 476

### Step 5: 4-(5-fluoro-6-(1-((4-isopropylphenyl)carbamoyl)piperidin-2-yl)pyridin-3-yl)benzoic acid

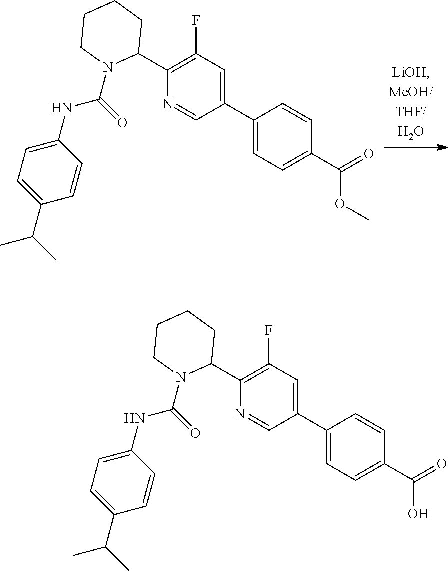

A mixture of methyl 4-(5-fluoro-6-(1-((4-isopropylphenyl)carbamoyl)piperidin-2-yl)pyridin-3-yl)benzoate (160 mg, 0.33 mmol) and LiOH monohydrate (122.6 mg, 2.92 mmol) in THF (5 mL) and H₂O (1 mL) was stirred at room temperature for 1 hour. Upon completion, the crude residue was directly purified by reversed phase flash chromatography (conditions: column, C18 silica gel; mobile phase A: Water (0.1% FA), mobile phase B: MeCN; Gradient (B %): 0 to 100% in 25 min; detector: UV 254 nm) to afford 4-(5-fluoro-6-(1-((4-isopropylphenyl)carbamoyl)piperidin-2-yl)pyridin-3-yl)benzoic acid (65 mg, 43% yield) as a white solid. LCMS (ESI) [M+H]⁺: 461.2

### Step 6: (S)-4-(5-fluoro-6-(1-((4-isopropylphenyl)carbamoyl)piperidin-2-yl)pyridin-3-yl)benzoic acid & (R)-4-(5-fluoro-6-(1-((4-isopropylphenyl)carbamoyl)piperidin-2-yl)pyridin-3-yl)benzoic acid

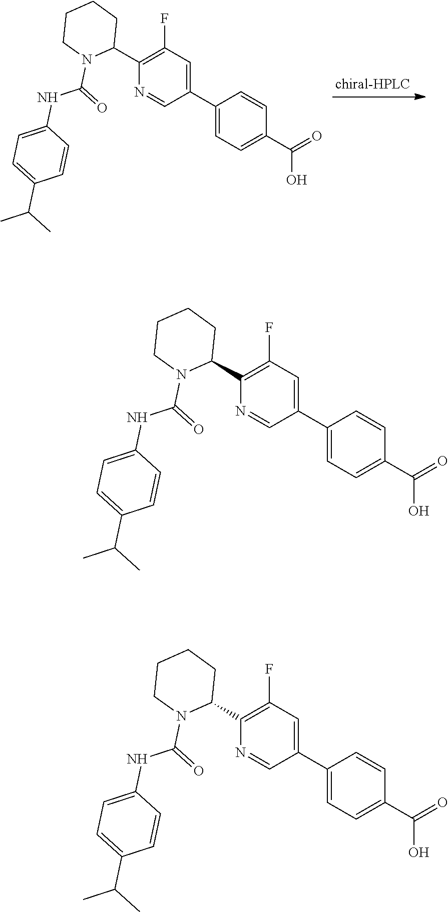 [SMILES: CC(C)c1ccc(NC(=O)N2CCCCC2c2ncc(-c3ccc(C(=O)O)cc3)cc2F)cc1 ; CC(C)c1ccc(NC(=O)N2CCCC[C@H]2c2ncc(-c3ccc(C(=O)O)cc3)cc2F)cc1 ; CC(C)c1ccc(NC(=O)N2CCCC[C@@H]2c2ncc(-c3ccc(C(=O)O)cc3)cc2F)cc1]

4-(5-fluoro-6-(1-((4-isopropylphenyl)carbamoyl)piperidin-2-yl)pyridin-3-yl)benzoic acid (65 mg, 0.141 mmol) was separated by prep Chiral-HPLC (conditions: Column: CHIRAL ART Cellulose-SC, 3*25 cm, 5 μm; Mobile Phase A: Hexane (0.1% FA), Mobile Phase B: EtOH; Flow rate: 40 mL/min; Gradient (B %): isocratic 30% B; Wave Length: 220/254 nm; RT1 (min): 6.5; RT2 (min): 8.6) to afford (Compound 71a; Method AA, Peak 1, 0.83 min) (S)-4-(5-fluoro-6-(1-((4-isopropylphenyl)carbamoyl)piperidin-2-yl)pyridin-3-yl)benzoic acid (1^{st} peak, 16.3 mg, 25% yield) and (Compound 71b; Method AA, Peak 2, 1.07 min) (R)-4-(5-fluoro-6-(1-((4-isopropylphenyl)carbamoyl)piperidin-2-yl)pyridin-3-yl)benzoic acid (2^{nd} peak, 15.9 mg, 24% yield) as a white solid.

| Cmpd # | Characterization Data |
|---|---|
| 71a | LCMS (ESI) [M + H]⁺: 462.1 |
| | ¹H NMR (400 MHz, DMSO-d₆) δ 12.98 (bs, 1H), 8.82-8.73 (m, 1H), 8.46 (s, 1H), 8.10 (d, J = 11.9 |
| | Hz, 1H), 8.03 (d, J = 8.0 Hz, 2H), 7.92 (d, J = 8.1 Hz, 2H), 7.33 (d, J = 8.6 Hz, 2H), 7.06 (d, J = 8.2 |
| | Hz, 2H), 5.84-5.57 (m, 1H), 4.14-3.99 (m, 1H), 3.66 (td, J = 10.7, 8.9, 5.1 Hz, 1H), 2.78 (hept, J = |
| | 6.9 Hz, 1H), 2.19-2.07 (m, 1H), 1.99-1.85 (m, 1H), 1.80-1.71 (m, 1H), 1.65-1.40 (m, 3H), 1.15 |
| | (d, J = 6.8 Hz, 6H). |
| 71b | LCMS (ESI) [M + H]⁺: 462.1 |
| | ¹H NMR (400 MHz, DMSO-d₆) δ 13.02 (bs, 1H), 8.77 (s, 1H), 8.46 (s, 1H), 8.10 (dd, J = 11.9, 2.0 |
| | Hz, 1H), 8.07-7.98 (m, 2H), 7.92 (d, J = 8.4 Hz, 2H), 7.33 (d, J = 8.6 Hz, 2H), 7.06 (d, J = 8.6 Hz, |
| | 2H), 5.79-5.69 (m, 1H), 4.06 (d, J = 12.6 Hz, 1H), 3.73-3.58 (m, 1H), 2.88-2.72 (m, 1H), 2.17- |
| | 2.08 (m, 1H), 2.01-1.87 (m, 1H), 1.83-1.71 (m, 1H), 1.68-1.38 (m, 3H), 1.15 (d, J = 6.8 Hz, 6H). |

### Example 72: (S)-4-(5-fluoro-6-(1-((3-methyl-4-(trifluoromethyl)phenyl)carbamoyl)pyrrolidin-2-yl)pyridin-3-yl)benzoic acid and (R)-4-(5-fluoro-6-(1-((3-methyl-4-(trifluoromethyl)phenyl)carbamoyl)pyrrolidin-2-yl)pyridin-3-yl)benzoic acid

### Step 1: methyl 4-(5-fluoro-6-(1-((3-methyl-4-(trifluoromethyl)phenyl)carbamoyl)pyrrolidin-2-yl)pyridin-3-yl)benzoate

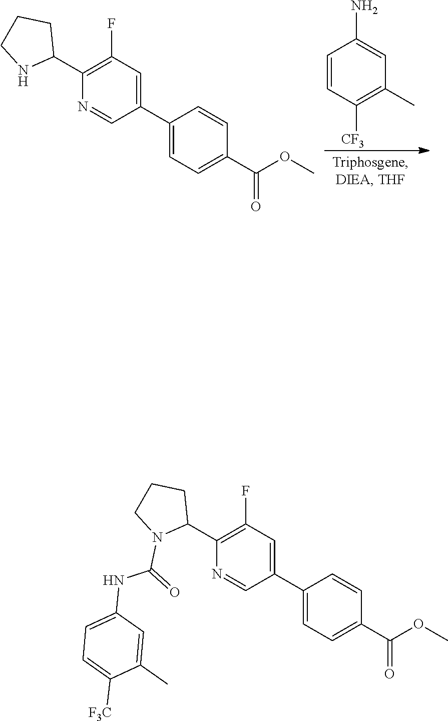 [SMILES: COC(=O)c1ccc(-c2cnc(C3CCCN3)c(F)c2)cc1 ; Cc1cc(N)ccc1C(F)(F)F ; COC(=O)c1ccc(-c2cnc(C3CCCN3C(=O)Nc3ccc(C(F)(F)F)c(C)c3)c(F)c2)cc1]

A mixture of 3-methyl-4-(trifluoromethyl)aniline (58 mg, 0.3 mmol), DIEA (129 mg, 0.9 mmol) and ditrichloromethyl carbonate (79 mg, 0.1 mmol) in THF (2 mL) was stirred at 0° C. under nitrogen atmosphere. After 10 minutes, methyl 4-[5-fluoro-6-(pyrrolidin-2-yl)pyridin-3-yl]benzoate (100 mg, 0.3 mmol) was added in portions at 0° C. The resulting mixture was warmed to room temperature and was allowed to stir for an additional 1 hour. Upon completion, the reaction was quenched with ice water. The resulting mixture was extracted with EtOAc (3×30 mL). The combined organic layers were washed with brine, dried over anhydrous Na₂SO₄, filtered and concentrated under reduced pressure. The crude residue was purified by silica gel column chromatography, eluted with PE/EA (1:1) to afford methyl 4-(5-fluoro-6-(1-((3-methyl-4-(trifluoromethyl)phenyl)carbamoyl)pyrrolidin-2-yl)pyridin-3-yl)benzoate (125 mg, 74% yield) as a yellow oil. LCMS (ESI) [M+H]⁺: 502

### Step 2: 4-(5-fluoro-6-(1-((3-methyl-4-(trifluoromethyl)phenyl)carbamoyl)pyrrolidin-2-yl)pyridin-3-yl)benzoic acid

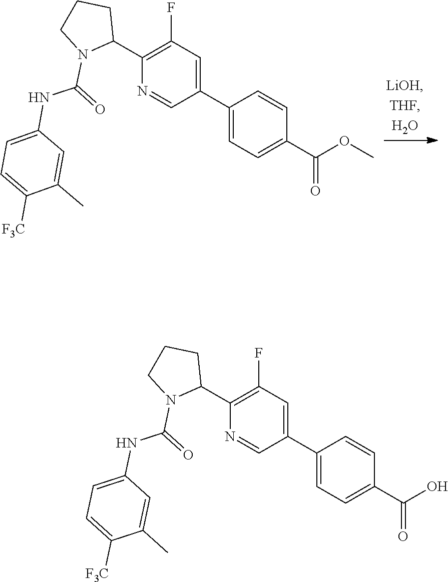 [SMILES: COC(=O)c1ccc(-c2cnc(C3CCCN3C(=O)Nc3ccc(C(F)(F)F)c(C)c3)c(F)c2)cc1 ; Cc1cc(NC(=O)N2CCCC2c2ncc(-c3ccc(C(=O)O)cc3)cc2F)ccc1C(F)(F)F]

A mixture of methyl 4-(5-fluoro-6-(1-((3-methyl-4-(trifluoromethyl)phenyl)carbamoyl)pyrrolidin-2-yl)pyridin-3-yl)benzoate (110 mg, 0.2 mmol) and LiOH monohydrate (42 mg, 1 mmol) in THF (1 mL) and H₂O (1 mL) was stirred at room temperature for 16 hours. Upon completion, the mixture was acidified to pH=3 with 1 M HCl (aq). The resulting mixture was extracted with EtOAc (3×20 mL). the combined organic layers were dried over anhydrous Na₂SO₄, filtered and concentrated under reduced pressure. The crude residue was purified by silica gel column chromatography, eluted with PE/EA (1:1) to afford 4-(5-fluoro-6-(1-((3-methyl-4-(trifluoromethyl)phenyl)carbamoyl)pyrrolidin-2-yl)pyridin-3-yl)benzoic acid (92 mg, 86% yield) as an off-white solid. LCMS (ESI) [M+H]⁺: 488

### Step 3: (S)-4-(5-fluoro-6-(1-((3-methyl-4-(trifluoromethyl)phenyl)carbamoyl)pyrrolidin-2-yl)pyridin-3-yl)benzoic acid and (R)-4-(5-fluoro-6-(1-((3-methyl-4-(trifluoromethyl)phenyl)carbamoyl)pyrrolidin-2-yl)pyridin-3-yl)benzoic acid

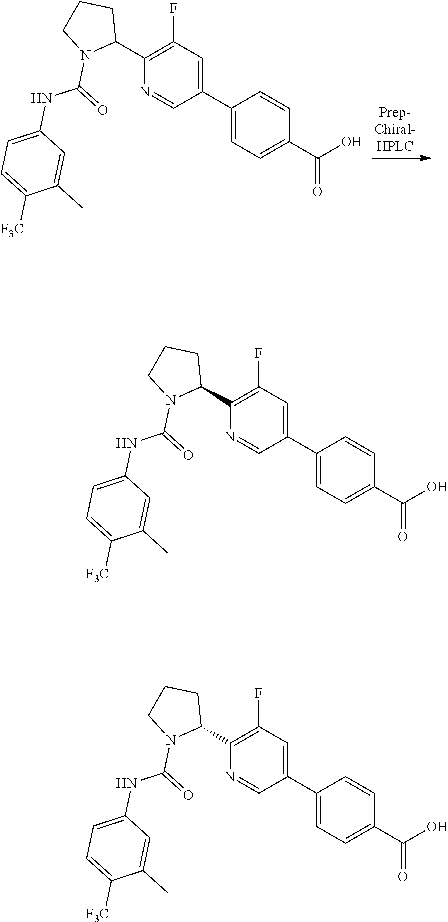 [SMILES: Cc1cc(NC(=O)N2CCCC2c2ncc(-c3ccc(C(=O)O)cc3)cc2F)ccc1C(F)(F)F ; Cc1cc(NC(=O)N2CCC[C@H]2c2ncc(-c3ccc(C(=O)O)cc3)cc2F)ccc1C(F)(F)F ; Cc1cc(NC(=O)N2CCC[C@@H]2c2ncc(-c3ccc(C(=O)O)cc3)cc2F)ccc1C(F)(F)F]

4-(5-fluoro-6-(1-((3-methyl-4-(trifluoromethyl)phenyl)carbamoyl)pyrrolidin-2-yl)pyridin-3-yl)benzoic acid (90 mg, 0.2 mmol) was purified by Prep-chiral HPLC (conditions: Column: CHIRALPAK IK 3*25 cm, 5 μm; Mobile Phase A: Hexane (0.1% formic acid), Mobile Phase B: EtOH; Flow rate: 40 mL/min; Gradient (B %): isocratic 20% B; Wave Length: 252/286 nm; RT1 (min): 7.08; RT2 (min): 10.68) to afford first peak (S)-4-(5-fluoro-6-(1-((3-methyl-4-(trifluoromethyl)phenyl)carbamoyl)pyrrolidin-2-yl)pyridin-3-yl)benzoic acid and (20.8 mg, Compound 72a; Method P, Peak 1, 0.90 min) as a white solid and second peak (R)-4-(5-fluoro-6-(1-((3-methyl-4-(trifluoromethyl)phenyl)carbamoyl)pyrrolidin-2-yl)pyridin-3-yl)benzoic acid (28.4 mg, Compound 72b; Method P, Peak 2, 1.26 min) as a white solid.

| Cmpd # | Characterization Data |
|---|---|
| 72a | LCMS (ESI) [M + H]⁺: 488.1 |
| | ¹H NMR (400 MHz, DMSO-d₆) δ 13.04 (bs, 1H), 8.73 (t, J = 1.6 Hz, 1H), 8.57 (s, 1H), 8.11-8.05 |
| | (m, 1H), 8.05-7.99 (m, 2H), 7.93-7.83 (m, 2H), 7.54 (s, 1H), 7.47 (s, 2H), 5.40-5.31 (m, 1H), |
| | 3.85-3.73 (m, 1H), 3.71-3.58 (m, 1H), 2.43-2.28 (m, 4H), 2.17-1.99 (m, 2H), 1.98-1.85 (m, |
| | 1H). |
| 72b | LCMS (ESI) [M + H]⁺: 488.2 |
| | ¹H NMR (400 MHz, DMSO-d₆) δ 13.08 (bs, 1H), 8.74 (s, 1H), 8.57 (s, 1H), 8.10-8.07 (m, 1H), |
| | 8.03 (d, J = 8.4 Hz, 2H), 7.90 (d, J = 8.4 Hz, 2H), 7.55 (s, 1H), 7.49 (s, 2H), 5.37-5.34 (m, 1H), |
| | 3.82-3.76 (m, 1H), 3.68-3.63 (m, 1H), 2.39-2.33 (m, 4H), 2.12-2.03 (m, 2H), 1.98-1.85 (m, |
| | 1H). |

### Example 73: (S)-4-(6-(1-((3-chloro-4-(trifluoromethyl)phenyl)carbamoyl)pyrrolidin-2-yl)-5-fluoropyridin-3-yl)benzoic acid and (R)-4-(6-(1-((3-chloro-4-(trifluoromethyl)phenyl)carbamoyl)pyrrolidin-2-yl)-5-fluoropyridin-3-yl)benzoic acid

### Step 1: methyl 4-(6-(1-((3-chloro-4-(trifluoromethyl)phenyl)carbamoyl)pyrrolidin-2-yl)-5-fluoropyridin-3-yl)benzoate

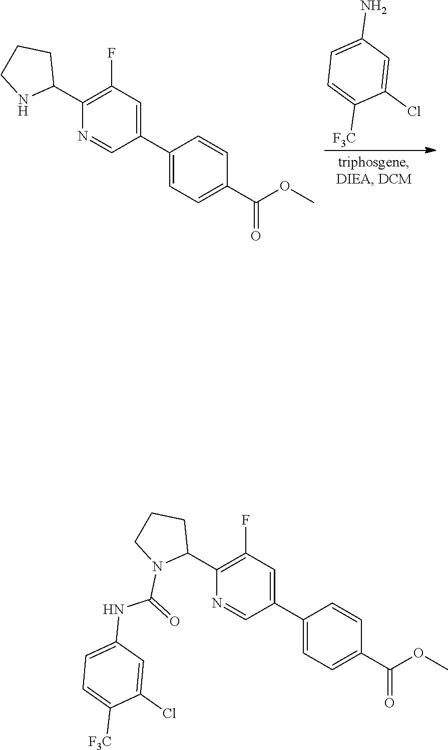 [SMILES: COC(=O)c1ccc(-c2cnc(C3CCCN3)c(F)c2)cc1 ; Nc1ccc(C(F)(F)F)c(Cl)c1 ; COC(=O)c1ccc(-c2cnc(C3CCCN3C(=O)Nc3ccc(C(F)(F)F)c(Cl)c3)c(F)c2)cc1]

A solution of 3-chloro-4-(trifluoromethyl)aniline (150 mg, 0.8 mmol) and DIEA (297 mg, 2.3 mmol) in DCM (5 mL) was treated with ditrichloromethyl carbonate (341 mg, 1.1 mmol) at 0° C. . After 10 minutes, methyl 4-[5-fluoro-6-(pyrrolidin-2-yl)pyridin-3-yl]benzoate (230 mg, 0.8 mmol) was added in portions. The resulting mixture was warmed to room temperature and was allowed to stir for an additional 30 minutes. Upon completion, the reaction was quenched with water. The resulting mixture was extracted with CH₂Cl₂ (3×50 mL). The combined organic layers were washed with brine (5×20 mL), dried over anhydrous Na₂SO₄, filtered and concentrated under reduced pressure. The crude residue was purified by silica gel column chromatography, eluted with PE/EA (2:1) to afford methyl 4-(6-(1-((3-chloro-4-(trifluoromethyl)phenyl)carbamoyl)pyrrolidin-2-yl)-5-fluoropyridin-3-yl)benzoate (160 mg, 40% yield) as an off-white solid. LCMS (ESI) [M+H]⁺: 522

### Step 2: 4-(6-(1-((3-chloro-4-(trifluoromethyl)phenyl)carbamoyl)pyrrolidin-2-yl)-5-fluoropyridin-3-yl)benzoic acid

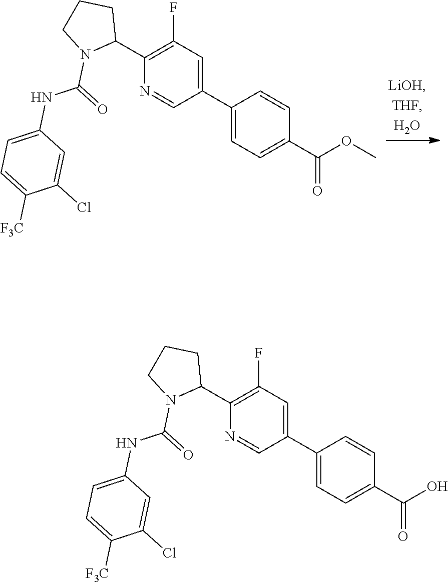 [SMILES: COC(=O)c1ccc(-c2cnc(C3CCCN3C(=O)Nc3ccc(C(F)(F)F)c(Cl)c3)c(F)c2)cc1 ; O=C(O)c1ccc(-c2cnc(C3CCCN3C(=O)Nc3ccc(C(F)(F)F)c(Cl)c3)c(F)c2)cc1]

To a stirred solution of methyl 4-(6-(1-((3-chloro-4-(trifluoromethyl)phenyl)carbamoyl)pyrrolidin-2-yl)-5-fluoropyridin-3-yl)benzoate (150 mg, 0.3 mmol) in THF (5 mL) was added LiOH monohydrate (59 mg, 1.4 mmol) in H₂O (1 mL) dropwise at 0° C. The resulting mixture was warmed to room temperature and stirred for 1 hour. Upon completion, the mixture was acidified to pH=5 with 1 M aq. HCl. The resulting mixture was extracted with EtOAc (3×10 mL). The combined organic layers were washed with brine (10 mL), dried over anhydrous Na₂SO₄, filtered and concentrated under reduced pressure. The crude residue was purified by silica gel column chromatography, eluted with PE/EA (1:5) to afford 4-(6-(1-((3-chloro-4-(trifluoromethyl)phenyl)carbamoyl)pyrrolidin-2-yl)-5-fluoropyridin-3-yl)benzoic acid (100 mg, 68% yield) as a white solid. LCMS (ESI) [M+H]⁺: 508

### Step 3: (S)-4-(6-(1-((3-chloro-4-(trifluoromethyl)phenyl)carbamoyl)pyrrolidin-2-yl)-5-fluoropyridin-3-yl)benzoic acid and (R)-4-(6-(1-((3-chloro-4-(trifluoromethyl)phenyl)carbamoyl)pyrrolidin-2-yl)-5-fluoropyridin-3-yl)benzoic acid

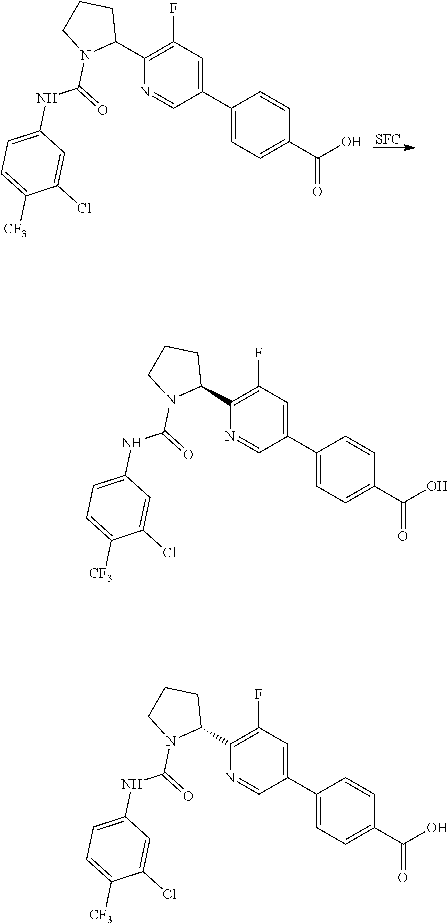

4-(6-(1-((3-chloro-4-(trifluoromethyl)phenyl)carbamoyl)pyrrolidin-2-yl)-5-fluoropyridin-3-yl)benzoic acid (90 mg) was purified by Prep-SFC (conditions Column: CHIRALPAK-IK, 3*25 mm, 5 μm; Mobile Phase A: CO₂, Mobile Phase B: MeOH; Flow rate: 80 mL/min; Gradient (B %): isocratic 35% B; Column Temperature (° C.): 25; Back Pressure (bar): 100; Wave Length: 254 nm; RT1 (min): 5.1; RT2 (min): 7.9) to afford first peak (S)-4-(6-(1-((3-chloro-4-(trifluoromethyl)phenyl)carbamoyl)pyrrolidin-2-yl)-5-fluoropyridin-3-yl)benzoic acid (38.5 mg, Compound 73a; Method AD, Peak 1, 1.38 min) as a white solid and second peak as (R)-4-(6-(1-((3-chloro-4-(trifluoromethyl)phenyl)carbamoyl)pyrrolidin-2-yl)-5-fluoropyridin-3-yl)benzoic acid (38.7 mg, Compound 73b; Method AD, Peak 2, 1.61 min) as a white solid.

| Cmpd # | Characterization Data |
|---|---|
| 73a | LCMS (ESI) [M + H]⁺: 508.1 |
| | ¹H NMR (400 MHz, DMSO-d₆) δ 13.14 (bs, 1H), 8.86 (s, 1H), 8.74 (s, 1H), 8.15-7.98 (m, 3H), |
| | 7.95-7.85 (m, 3H), 7.70-7.56 (m, 2H), 5.36 (t, J = 5.9 Hz, 1H), 3.85-3.74 (m, 1H), 3.73-3.60 |
| | (m, 1H), 2.45-2.29 (m, 1H), 2.20-1.98 (m, 2H), 1.97-1.85 (m, 1H). |
| 73b | LCMS (ESI) [M + H]⁺: 508.1 |
| | ¹H NMR (400 MHz, DMSO-d₆) δ 13.18 (bs, 1H), 8.86 (s, 1H), 8.74 (s, 1H), 8.10-8.02 (m, 3H), |
| | 7.91-7.87 (m, 3H), 7.67-7.60 (m, 2H), 5.39-5.36 (m, 1H), 3.82-3.70 (m, 1H), 3.68-3.64 (m, |
| | 1H), 2.42-2.36 (m, 1H), 2.17-2.01 (m, 2H), 1.95-1.88 (m, 1H). |

### Example 77: (S)-4-(2-(1-((4-isopropylphenyl)carbamoyl)pyrrolidin-2-yl)benzo[d]oxazol-6-yl)benzoic acid and (R)-4-(2-(1-((4-isopropylphenyl)carbamoyl)pyrrolidin-2-yl)benzo[d]oxazol-6-yl)benzoic acid

### Step 1: tert-butyl (R)-2-((4-bromo-2-hydroxyphenyl)carbamoyl)pyrrolidine-1-carboxylate

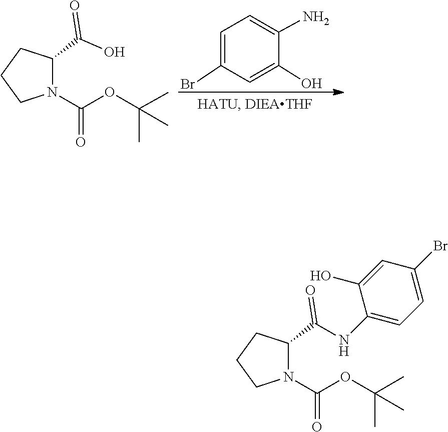

A solution of (tert-butoxycarbonyl)-D-proline (1 g, 4.6 mmol) in THF (10 mL) was treated with HATU (2.12 g, 5.5 mmol) and DIEA (900 mg, 6.9 mmol) at 0° C. under nitrogen atmosphere. After 5 minutes, 2-amino-5-bromophenol (873 mg, 4.6 mmol) was added. The resulting mixture was warmed to room temperature and stirred for 2 hours. Upon completion, the reaction mixture was diluted with EtOAc. The organic layer was washed with water (30 mL), dried over anhydrous Na₂SO₄, filtered and concentrated under reduced pressure. The crude residue was purified by reversed-phase flash chromatography (conditions: column: C18 silica gel; mobile phase A: water (0.1% formic acid), mobile phase B: MeCN; Gradient (B %): 10% to 50% in 10 min; detector: UV 254 nm) to afford tert-butyl (R)-2-((4-bromo-2-hydroxyphenyl)carbamoyl)pyrrolidine-1-carboxylate (1.5 g, 83% yield) as a white solid. LCMS (ESI) [M+H]⁺: 385

### Step 2: tert-butyl (R)-2-(6-bromobenzo[d]oxazol-2-yl)pyrrolidine-1-carboxylate

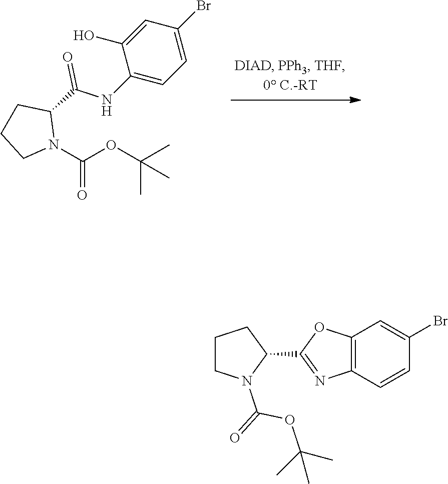 [SMILES: CC(C)(C)OC(=O)N1CCC[C@@H]1C(=O)Nc1ccc(Br)cc1O ; CC(C)(C)OC(=O)N1CCC[C@@H]1c1nc2ccc(Br)cc2o1]

A solution of tert-butyl (R)-2-((4-bromo-2-hydroxyphenyl)carbamoyl)pyrrolidine-1-carboxylate (500 mg, 1.2 mmol) in THF (4 mL) was treated with PPh₃ (340 mg, 1.2 mmol) at room temperature under nitrogen atmosphere. After 5 minutes, the reaction was cooled to 0° C. and DIAD (262 mg, 1.2 mmol) was added dropwise. The resulting mixture was warmed to room temperature and stirred for 12 hours. Upon completion, the resulting mixture was concentrated under reduced pressure. The crude residue was directly purified by silica gel column chromatography, eluted with PE/EA (1:1) to afford tert-butyl (R)-2-(6-bromobenzo[d]oxazol-2-yl)pyrrolidine-1-carboxylate (360 mg, 75% yield) as a light yellow solid. LCMS (ESI) [M+H]⁺: 367

### Step 3: tert-butyl (R)-2-(6-(4-(tert-butoxycarbonyl)phenyl)benzo[d]oxazol-2-yl)pyrrolidine-1-carboxylate

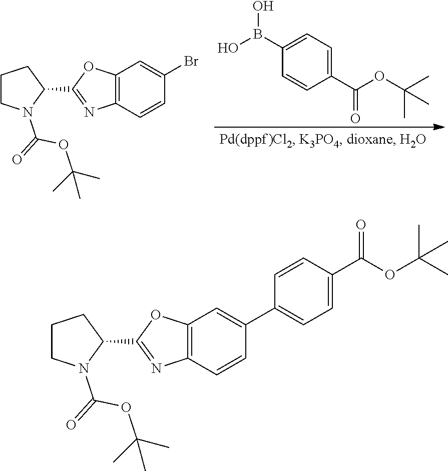 [SMILES: CC(C)(C)OC(=O)N1CCC[C@@H]1c1nc2ccc(Br)cc2o1 ; CC(C)(C)OC(=O)c1ccc(B(O)O)cc1 ; CC(C)(C)OC(=O)c1ccc(-c2ccc3nc([C@H]4CCCN4C(=O)OC(C)(C)C)oc3c2)cc1]

A mixture of tert-butyl (R)-2-(6-bromobenzo[d]oxazol-2-yl)pyrrolidine-1-carboxylate (350 mg, 0.9 mmol) and 4-(tert-butoxycarbonyl)phenylboronic acid (423 mg, 1.9 mmol), Pd(dppf)Cl₂ (69 mg, 95 μmol), K₃PO₄ (606 mg, 2.8 mmol) in 1,4-dioxane (10 mL) and H₂O (1 mL) was stirred at 80° C. for 2 hours under nitrogen atmosphere. Upon completion, the resulting mixture was diluted with water (10 mL) and extracted with EtOAc (3×50 mL). The combined organic layers were washed with water (3×10 mL), dried over anhydrous Na₂SO₄, filtered and concentrated under reduced pressure. The crude residue was purified by silica gel column chromatography, eluted with PE/EA (1:1) to afford tert-butyl (R)-2-(6-(4-(tert-butoxycarbonyl)phenyl)benzo[d]oxazol-2-yl)pyrrolidine-1-carboxylate (410 mg, 92% yield) as a light yellow solid. LCMS [M+H]⁺: 465

### Step 4: 4-(2-(pyrrolidin-2-yl)benzo[d]oxazol-6-yl)benzoic acid

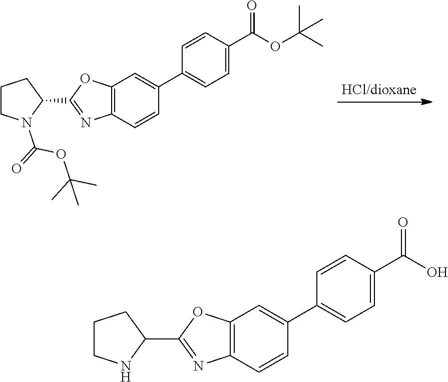 [SMILES: CC(C)(C)OC(=O)c1ccc(-c2ccc3nc([C@H]4CCCN4C(=O)OC(C)(C)C)oc3c2)cc1 ; O=C(O)c1ccc(-c2ccc3nc(C4CCCN4)oc3c2)cc1]

A mixture of tert-butyl (R)-2-(6-(4-(tert-butoxycarbonyl)phenyl)benzo[d]oxazol-2-yl)pyrrolidine-1-carboxylate (350 mg, 0.7 mmol) in 4.0 M HCl in 1,4-dioxane (4 mL) was stirred at room temperature for 12 hours. Upon completion, the resulting mixture was concentrated under reduced pressure to afford 4-(2-(pyrrolidin-2-yl)benzo[d]oxazol-6-yl)benzoic acid (230 mg as a racemate, crude) as a light yellow solid, which was used directly in the next step without further purification. LCMS (ESI) [M+H]⁺: 309

### Step 5: 4-(2-(1-((4-isopropylphenyl)carbamoyl)pyrrolidin-2-yl)benzo[d]oxazol-6-yl)benzoic acid

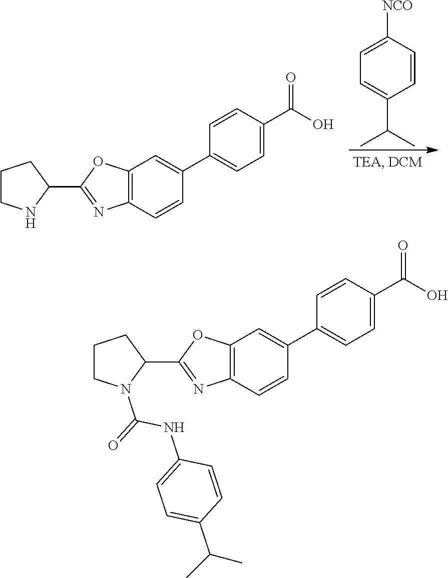

A solution of 4-(2-(pyrrolidin-2-yl)benzo[d]oxazol-6-yl)benzoic acid (150 mg, 0.4 mmol) in DCM (2 mL) was treated with TEA (49 mg, 0.4 mmol) at room temperature under nitrogen atmosphere. After 5 minutes, the reaction was cooled to 0° C. and 1-isocyanato-4-isopropylbenzene (78 mg, 0.4 mmol) was added. The resulting mixture was warmed to room temperature and stirred for 1 hour. Upon completion, the reaction was quenched with ice water at 0° C. The resulting mixture was extracted with EtOAc (3×20 mL). The combined organic layers were washed with water (3×5 mL), dried over anhydrous Na₂SO₄, filtered and concentrated under reduced pressure. The crude residue was purified by reversed-phase flash chromatography (conditions: column: C18 silica gel; mobile phase A: water (0.1% formic acid), mobile phase B: MeCN; Gradient (B %): 10% to 50% in 10 min; detector: UV 254 nm) to afford 4-(2-(1-((4-isopropylphenyl)carbamoyl)pyrrolidin-2-yl)benzo[d]oxazol-6-yl)benzoic acid (113 mg) as a white solid. LCMS (ESI) [M+H]⁺: 470

### Step 5: (S)-4-(2-(1-((4-isopropylphenyl)carbamoyl)pyrrolidin-2-yl)benzo[d]oxazol-6-yl)benzoic acid and (R)-4-(2-(1-((4-isopropylphenyl)carbamoyl)pyrrolidin-2-yl)benzo[d]oxazol-6-yl)benzoic acid

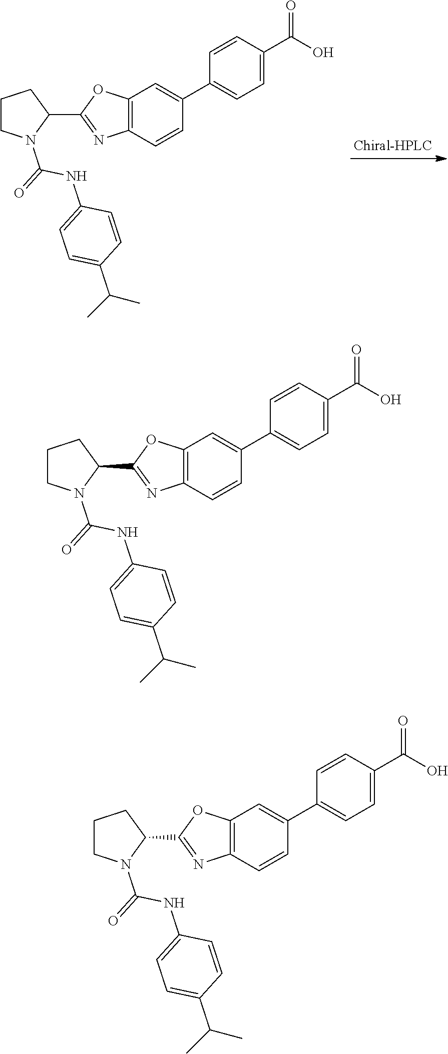 [SMILES: CC(C)c1ccc(NC(=O)N2CCCC2c2nc3ccc(-c4ccc(C(=O)O)cc4)cc3o2)cc1 ; CC(C)c1ccc(NC(=O)N2CCC[C@H]2c2nc3ccc(-c4ccc(C(=O)O)cc4)cc3o2)cc1 ; CC(C)c1ccc(NC(=O)N2CCC[C@@H]2c2nc3ccc(-c4ccc(C(=O)O)cc4)cc3o2)cc1]

4-(2-(1-((4-isopropylphenyl)carbamoyl)pyrrolidin-2-yl)benzo[d]oxazol-6-yl)benzoic acid (111 mg) was purified by prep Chiral-HPLC (conditions: Column: CHIRAL ART Cellulose-SC, 3*25 cm, 5 μm; Mobile Phase A: Hexane (0.1% formic acid), Mobile Phase B: EtOH; Flow rate: 40 mL/min; Gradient (B %): isocratic 30% B; Wave Length: 220/254 nm; RT1 (min): 11.45; RT2 (min): 14.68) to afford first peak (S)-4-(2-(1-((4-isopropylphenyl)carbamoyl)pyrrolidin-2-yl)benzo[d]oxazol-6-yl)benzoic acid (13.5 mg, Compound 77a; Method AA, Peak 1, 1.58 min) as a white solid and second peak (R)-4-(2-(1-((4-isopropylphenyl)carbamoyl)pyrrolidin-2-yl)benzo[d]oxazol-6-yl)benzoic acid (68.9 mg, Compound 77b; Method AA, Peak 2, 2.12 min) as a white solid.

| Cmpd # | Characterization Data |
|---|---|
| 77a | LCMS (ESI) [M + H]⁺: 470.2 |
| | ¹H NMR (400 MHz, DMSO-d₆) δ 12.99 (bs, 1H), 8.34 (s, 1H), 8.09 (s, 1H), 8.02 (d, J = 8.0 Hz, |
| | 2H), 7.86 (d, J = 8.0 Hz, 2H), 7.81-7.69 (m, 2H), 7.36 (d, J = 8.1 Hz, 2H), 7.07 (d, J = 8.2 Hz, |
| | 2H), 5.30-5.23 (m, 1H), 3.80-3.74 (m, 1H), 3.60-3.57 (m, 1H), 2.82-2.75 (m, 1H), 2.43-2.34 |
| | (m, 1H), 2.13-2.09 (m, 3H), 1.15 (d, J = 6.9 Hz, 6H). |
| 77b | LCMS (ESI) [M + H]⁺: 470.1 |
| | ¹H NMR (400 MHz, DMSO-d₆) δ 12.99 (bs, 1H), 8.34 (s, 1H), 8.09 (s, 1H), 8.02 (d, J = 8.4 Hz, |
| | 2H), 7.87-7.72 (m, 2H), 7.73 (d, J = 9.2 Hz, 2H), 7.36 (d, J = 8.4 Hz, 2H), 7.07 (d, J = 8.4 Hz, |
| | 2H), 5.27-5.25 (m, 1H), 3.78-3.75 (m, 1H), 3.63-3.57 (m, 1H), 2.82-2.70 (m, 1H), 2.41-2.38 |
| | (m, 1H), 2.17-2.06 (m, 3H), 1.15 (d, J = 6.8 Hz, 6H). |

### Example 80: (R)-5-(4-(2-((4-isopropylphenyl)carbamoyl)pyrrolidine-1-carboxamido)phenyl)-6-methylpicolinic acid

### Step 1: methyl 5-(4-aminophenyl)-6-methylpicolinate

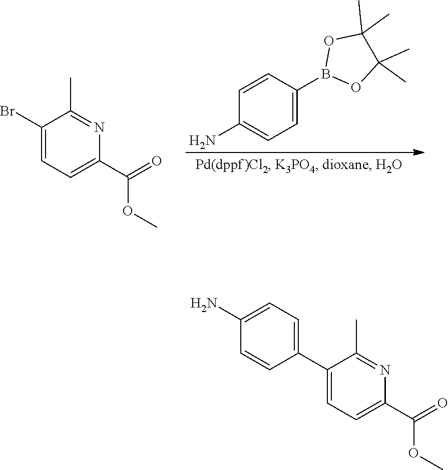 [SMILES: COC(=O)c1ccc(Br)c(C)n1 ; CC1(C)OB(c2ccc(N)cc2)OC1(C)C ; COC(=O)c1ccc(-c2ccc(N)cc2)c(C)n1]

A mixture of methyl 5-bromo-6-methylpyridine-2-carboxylate (500 mg, 2.1 mmol) and 4-(4,4,5,5-tetramethyl-1,3,2-dioxaborolan-2-yl)aniline (571 mg, 2.6 mmol), Pd(dppf)Cl₂ (159 mg, 0.2 mmol), K₃PO₄ (1.3 g, 6.5 mmol) in dioxane (10 mL) and H₂O (1 mL) was stirred at 80° C. for 2 hours under nitrogen atmosphere. Upon completion, the resulting mixture was diluted with water and extracted with EtOAc (3×30 mL). The combined organic layers were washed with water (3×5 mL), dried over anhydrous Na₂SO₄, filtered and concentrated under reduced pressure. The crude residue was purified by reversed-phase flash chromatography (conditions: column: C18 silica gel; mobile phase A: water (0.1% formic acid), mobile phase B: 10% to 50% in 10 min; detector: UV 254 nm) to afford methyl 5-(4-aminophenyl)-6-methylpicolinate (300 mg, 56% yield) as a brown solid. LCMS (ESI) [M+H]⁺: 243

### Step 2: methyl (R)-5-(4-(2-((4-isopropylphenyl)carbamoyl)pyrrolidine-1-carboxamido)phenyl)-6-methylpicolinate

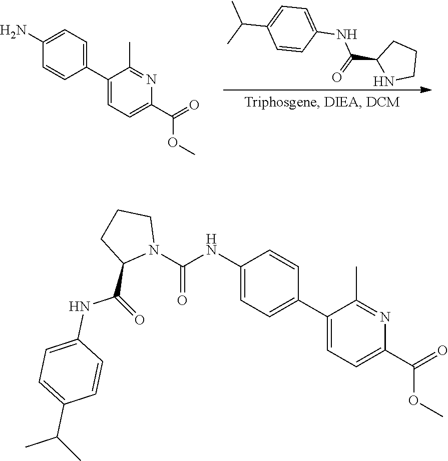 [SMILES: COC(=O)c1ccc(-c2ccc(N)cc2)c(C)n1 ; CC(C)c1ccc(NC(=O)[C@H]2CCCN2)cc1 ; COC(=O)c1ccc(-c2ccc(NC(=O)N3CCC[C@@H]3C(=O)Nc3ccc(C(C)C)cc3)cc2)c(C)n1]

A solution of triphosgene (73 mg, 0.2 mmol) in DCM (4 mL) was treated with (R)—N-(4-isopropylphenyl)pyrrolidine-2-carboxamide (143 mg, 0.6 mmol) and DIEA (240 mg, 1.8 mmol) at 0° C. for 15 min under nitrogen atmosphere followed by the addition of methyl 5-(4-aminophenyl)-6-methylpicolinate (150 mg, 0.6 mmol) at room temperature. The resulting mixture was stirred at room temperature for 2 hours. The reaction was quenched by the addition of sat. aq. NH₄Cl (2 mL) at 0° C. The resulting mixture was extracted with EtOAc (3×15 mL). The combined organic layers were washed with water, dried over anhydrous Na₂SO₄, filtered and concentrated under reduced pressure. The crude residue was purified by silica gel column chromatography, eluted with CH₂Cl₂/MeOH (5:1) to afford methyl (R)-5-(4-(2-((4-isopropylphenyl)carbamoyl)pyrrolidine-1-carboxamido)phenyl)-6-methylpicolinate (150 mg, 48% yield) as a white solid. LCMS (ESI) [M+H]⁺: 501

### Step 3: (R)-5-(4-(2-((4-isopropylphenyl)carbamoyl)pyrrolidine-1-carboxamido)phenyl)-6-methylpicolinic acid

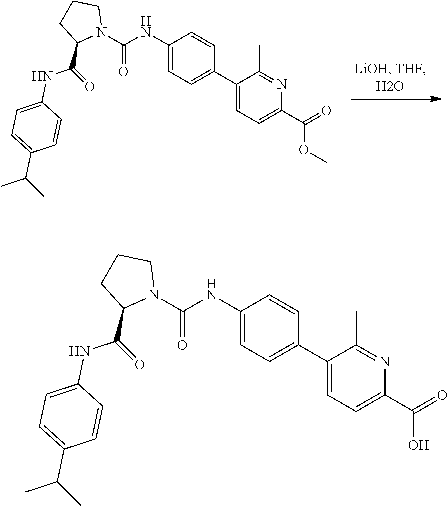

A mixture of methyl (R)-5-(4-(2-((4-isopropylphenyl)carbamoyl)pyrrolidine-1-carboxamido)phenyl)-6-methylpicolinate (140 mg, 0.2 mmol) and LiOH monohydrate (34 mg, 0.8 mmol) in THF (2 mL) and H₂O (2 mL) was stirred at room temperature for 2 hours. Upon completion, the mixture was acidified to pH=6 with HCl (1M, aq.). The resulting mixture was extracted with EtOAc (3×10 mL). The combined organic layers were washed with brine (10 mL), dried over anhydrous Na₂SO₄, filtered and concentrated under reduced pressure. The resulting mixture was concentrated under reduced pressure. The crude residue was purified by Prep-HPLC (conditions: Column: XSelect CSH Fluoro Phenyl 30*150 mm, 5 μm; Mobile Phase A: Water (0.1% formic acid), Mobile Phase B: MeCN; Flow rate: 60 mL/min; Gradient (B %): 25% B to 45% B in 15 min; Wave Length: 254 nm/220 nm; RT1 (min): 9.12 min) to afford (R)-5-(4-(2-((4-isopropylphenyl)carbamoyl)pyrrolidine-1-carboxamido)phenyl)-6-methylpicolinic acid (43.9 mg, 32% yield) as an off-white solid.

| Cmpd # | Characterization Data |
|---|---|
| 80 | LCMS (ESI) [M + H]⁺: 487.3 |
| | ¹H NMR (400 MHz, DMSO-d₆) δ 9.90 (s, 1H), 8.44 (s, 1H), 7.91 (d, J = 7.9 Hz, 1H), 7.75 (d, J = |
| | 7.9 Hz, 1H), 7.65 (d, J = 8.4 Hz, 2H), 7.51 (d, J = 8.4 Hz, 2H), 7.32 (d, J = 8.4 Hz, 2H), 7.15 (d, J = |
| | 8.8 Hz, 2H), 4.49-4.46 (m, 1H), 3.68-3.65 (m, 1H), 3.57-3.51 (m, 1H), 2.86-2.79 (m, 1H), |
| | 2.50 (s, 3H), 2.18-2.12 (m, 1H), 2.10-1.86 (m, 3H), 1.17 (d, J = 6.9 Hz, 6H). |

### Example 82: (R)-4′-(2-((4-isopropylphenyl)carbamoyl)pyrrolidine-1-carboxamido)-3-methoxy-[1,1′-biphenyl]-4-carboxylic acid

### Step 1: methyl 4′-amino-3-methoxy-[1,1′-biphenyl]-4-carboxylate

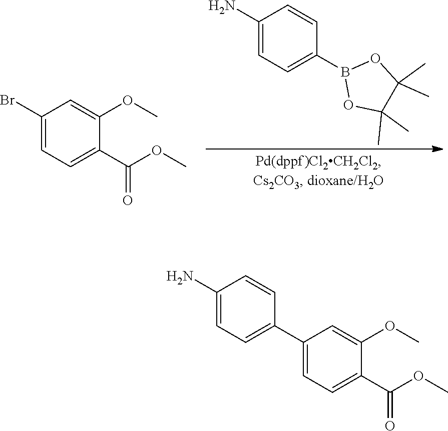 [SMILES: COC(=O)c1ccc(Br)cc1OC ; CC1(C)OB(c2ccc(N)cc2)OC1(C)C ; COC(=O)c1ccc(-c2ccc(N)cc2)cc1OC]

A mixture of methyl 4-bromo-2-methoxybenzoate (500 mg, 2.0 mmol), 4-(4,4,5,5-tetramethyl-1,3,2-dioxaborolan-2-yl)aniline (447 mg, 2.0 mmol), Pd(dppf)Cl₂·CH₂Cl₂ (333 mg, 0.4 mmol) and Cs₂CO₃ (1.99 g, 6.1 mmol) in 1,4-dioxane (16 mL) and H₂O (4 mL) was stirred at 90° C. for 1 hour under nitrogen atmosphere. Upon completion, the resulting mixture was concentrated under reduced pressure. The crude residue was purified by reversed phase flash chromatography (conditions: column: C18 silica gel; mobile phase A: water (0.1% formic acid), mobile phase B: MeCN; Gradient (B %): 0% to 100% in 20 min; detector, UV 254 nm) to afford methyl 4′-amino-3-methoxy-[1,1′-biphenyl]-4-carboxylate (470 mg, 90% yield) as a yellow solid. LCMS (ESI) [M+H]⁺: 258

### Step 2: tert-butyl (R)-2-((4-isopropylphenyl)carbamoyl)pyrrolidine-1-carboxylate

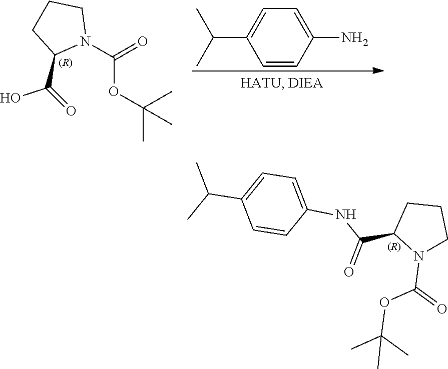

To a mixture of (tert-butoxycarbonyl)-D-proline (1.0 g, 4.6 mmol) and HATU (2.1 g, 5.6 mmol) in DCM (15 mL) was added DIEA (0.66 g, 5.1 mmol) at 0° C. under nitrogen atmosphere. After 30 minutes 4-isopropylaniline (0.69 g, 5.1 mmol) was added. The resulting mixture was warmed to room temperature and was allowed to stir for an additional 4 hours. Upon completion, the resulting mixture was concentrated under reduced pressure. The crude residue was directly purified by reversed phase flash chromatography (conditions: column: C18 silica gel; mobile phase A: water (0.1% formic acid), mobile phase B: MeCN; Gradient (B %): 0% to 100% in 20 min; detector: UV 254 nm) to afford tert-butyl (R)-2-((4-isopropylphenyl)carbamoyl)pyrrolidine-1-carboxylate (1.1 g, 71% yield) as a white solid. LCMS (ESI) [M+H]⁺: 333

### Step 3: (R)—N-(4-isopropylphenyl)pyrrolidine-2-carboxamide

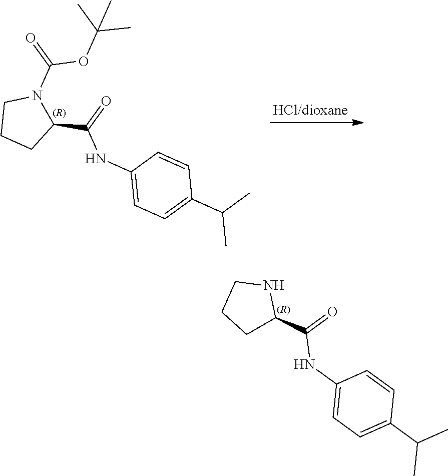

A mixture of tert-butyl (R)-2-((4-isopropylphenyl)carbamoyl)pyrrolidine-1-carboxylate (1.0 g, 3.0 mmol) in 4.0 M HCl in 1,4-dioxane (10 mL) was stirred at room temperature overnight. Upon completion, the resulting mixture was concentrated under reduced pressure to afford (R)—N-(4-isopropylphenyl)pyrrolidine-2-carboxamide (800 mg, crude) as an off-white solid, which was used directly in the next step without further purification. LCMS (ESI) [M+H]⁺: 233

### Step 4: methyl (R)-4′-(2-((4-isopropylphenyl)carbamoyl)pyrrolidine-1-carboxamido)-3-methoxy-[1,1′-biphenyl]-4-carboxylate

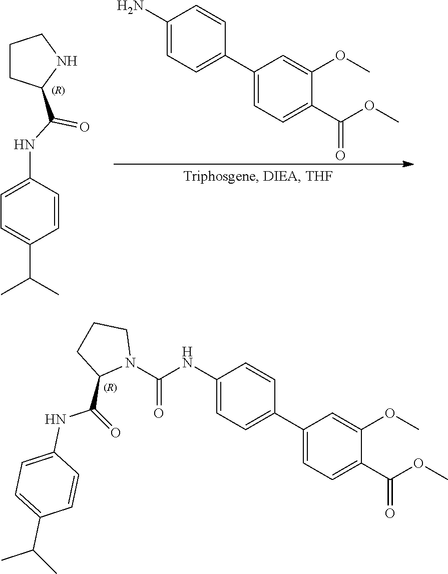

To a mixture of ditrichloromethyl carbonate (102 mg, 0.3 mmol) in DCM (5 mL) was added methyl 4′-amino-3-methoxy-[1,1′-biphenyl]-4-carboxylate (222 mg, 0.9 mmol) and DIEA (556 mg, 4.3 mmol) at 0° C. under nitrogen atmosphere. After 30 minutes, (R)—N-(4-isopropylphenyl)pyrrolidine-2-carboxamide (200 mg, 0.9 mmol) was added dropwise. The resulting mixture was warmed to room temperature and was allowed to stir for an additional 1 hour. Upon completion, the resulting mixture was concentrated under reduced pressure. The crude residue was directly purified by reversed phase flash chromatography (conditions: column: C18 silica gel; mobile phase A: water (0.1% formic acid), mobile phase b: MeCN; Gradient (B %): 0% to 100% in 20 min; detector: UV 254 nm) to afford methyl (R)-4′-(2-((4-isopropylphenyl)carbamoyl)pyrrolidine-1-carboxamido)-3-methoxy-[1,1′-biphenyl]-4-carboxylate (300 mg, 68% yield) as a white solid. LCMS (ESI) [M+H]⁺: 516

### Step 5: (R)-4′-(2-((4-isopropylphenyl)carbamoyl)pyrrolidine-1-carboxamido)-3-methoxy-[1,1′-biphenyl]-4-carboxylic acid

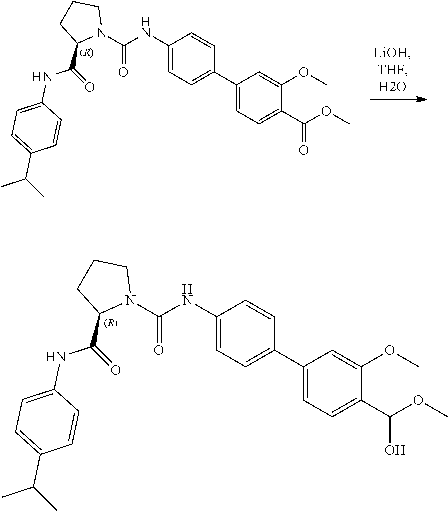

A mixture of methyl (R)-4′-(2-((4-isopropylphenyl)carbamoyl)pyrrolidine-1-carboxamido)-3-methoxy-[1,1′-biphenyl]-4-carboxylate (120 mg, 0.2 mmol) and LiOH monohydrate (50 mg, 1.2 mmol) in THF (1 mL) and H₂O (1 mL) was stirred at 50° C. for 5 hours. Upon completion, the resulting mixture was concentrated under reduced pressure. The crude residue was purified by Prep-HPLC (conditions: Column: Xselect CSH Prep C18, 30*150 mm 5 m; Mobile Phase A: water (0.1% formic acid), Mobile Phase B: ACN; Flow rate: 60 mL/min; Gradient (B %): 39% to 59% B in 10 min; Wave Length: 254 nm/220 nm; RT1 (min): 11.30 min) to afford (R)-4′-(2-((4-isopropylphenyl)carbamoyl)pyrrolidine-1-carboxamido)-3-methoxy-[1,1′-biphenyl]-4-carboxylic acid (49.5 mg, 42% yield) as a white solid.

| Cmpd # | Characterization Data |
|---|---|
| 82 | LCMS (ESI) [M + H]⁺: 502.2 |
| | ¹H NMR (400 MHz, DMSO-d₆) δ 12.46 (bs, 1H), 9.92 (bs, 1H), 8.44 (bs, 1H), 7.76-7.46 (m, 7H), |
| | 7.33-7.16 (m, 4H), 4.48-4.46 (m, 1H), 3.91 (bs, 3H), 3.79-3.68 (m, 1H), 3.58-3.52 (m, 1H), |
| | 2.83-2.79 (m, 1H), 2.19-2.11 (m, 1H), 2.08-1.88 (m, 3H), 1.17 (d, J = 6.8 Hz, 6H). |

### Example 83: (R)-3-(2-(1-((4-isopropylphenyl)carbamoyl)pyrrolidin-2-yl)benzo[d]oxazol-5-yl)benzoic acid

### Step 1: tert-butyl (R)-2-(5-(3-(tert-butoxycarbonyl)phenyl)benzo[d]oxazol-2-yl)pyrrolidine-1-carboxylate

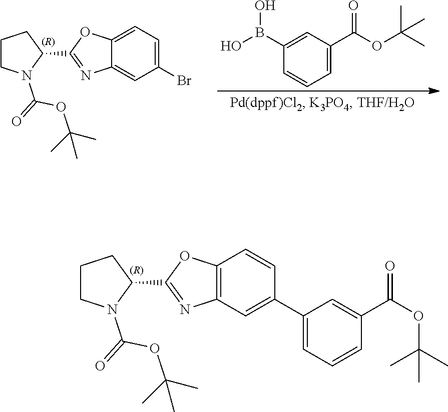

A mixture of tert-butyl (R)-2-(5-bromobenzo[d]oxazol-2-yl)pyrrolidine-1-carboxylate (300 mg, 0.8 mmol), 3-(tert-butoxycarbonyl)phenylboronic acid (362 mg, 1.6 mmol), Pd(dppf)Cl₂·CH₂Cl₂ (133 mg, 0.2 mmol) and K₃PO₄ (520 mg, 2.4 mmol) in THF (8 mL) and H₂O (2 mL) was stirred at 90° C. for 1 hour under nitrogen atmosphere. Upon completion, the resulting mixture was diluted with water (10 mL). The resulting mixture was extracted with EtOAc (3×15 mL). The combined organic layers were dried over anhydrous Na₂SO₄, filtered and concentrated under reduced pressure. The crude residue was purified by reversed phase flash chromatography (conditions: column: C18 silica gel; mobile phase A: water (0.1% formic acid), mobile phase B: MeCN; Gradient (B %): 0% to 100% in 20 min; detector, UV 254 nm) to afford tert-butyl (R)-2-(5-(3-(tert-butoxycarbonyl)phenyl)benzo[d]oxazol-2-yl)pyrrolidine-1-carboxylate (280 mg, 74% yield) as an off-white solid. LCMS (ESI) [M+H]⁺: 465

### Step 2: (R)-3-(2-(pyrrolidin-2-yl)benzo[d]oxazol-5-yl)benzoic acid

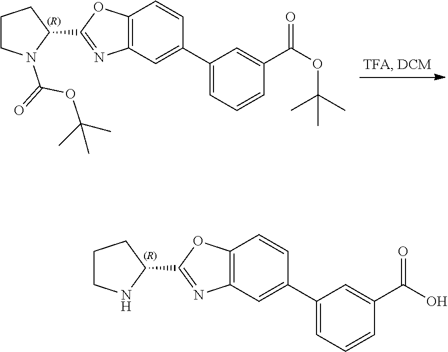

A mixture of tert-butyl (R)-2-(5-(3-(tert-butoxycarbonyl)phenyl)benzo[d]oxazol-2-yl)pyrrolidine-1-carboxylate (200 mg, 0.4 mmol) in TFA (1.5 mL) and DCM (5 mL) was stirred at room temperature for 3 hours. Upon completion, the resulting mixture was concentrated under reduced pressure to afford (R)-3-(2-(pyrrolidin-2-yl)benzo[d]oxazol-5-yl)benzoic acid (160 mg, crude) as a yellow solid, which was used in the next step without further purification. LCMS (ESI) [M+H]⁺: 309

### Step 3: (R)-3-(2-(1-((4-isopropylphenyl)carbamoyl)pyrrolidin-2-yl)benzo[d]oxazol-5-yl)benzoic acid

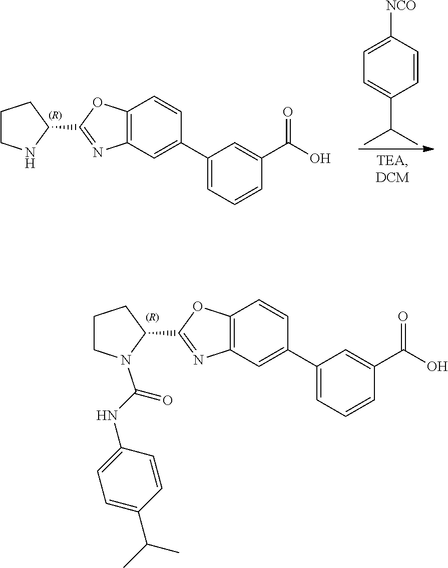

To a mixture of (R)-3-(2-(pyrrolidin-2-yl)benzo[d]oxazol-5-yl)benzoic acid (140 mg, 0.5 mmol) and Et₃N (46 mg, 0.5 mmol) in DCM (3 mL) was added 1-isocyanato-4-isopropylbenzene (73 mg, 0.5 mmol) at 0° C. under nitrogen atmosphere. The resulting mixture was stirred at room temperature for 1 hour. Upon completion, the resulting mixture was concentrated under reduced pressure. The crude residue was purified by reversed phase flash chromatography (conditions: column: C18 silica gel; Mobile Phase A: water (0.1% formic acid), Mobile Phase B: ACN; Flow rate: 35 mL/min; Gradient (B %): 45% B to 55% B in 10 min; Wave Length: 254 nm/220 nm) to afford (R)-3-(2-(1-((4-isopropylphenyl)carbamoyl)pyrrolidin-2-yl)benzo[d]oxazol-5-yl)benzoic acid (51.8 mg, 24% yield) as a white solid.

| Cmpd # | Characterization Data |
|---|---|
| 83 | LCMS (ESI) [M + H]⁺: 470.3 |
| | ¹H NMR (400 MHz, DMSO-d₆) δ 13.09 (bs, 1H), 8.34 (s, 1H), 8.19 (s, 1H), 8.01-7.91 (m, 3H), |
| | 7.80 (d, J = 8.5 Hz, 1H), 7.71-7.64 (m, 1H), 7.61 (t, J = 7.7 Hz, 1H), 7.36 (d, J = 8.4 Hz, 2H), 7.07 |
| | (d, J = 8.3 Hz, 2H), 5.30-5.23 (m, 1H), 3.83-3.73 (m, 1H), 3.65-3.55 (m, 1H), 2.86-2.71 (m, |
| | 1H), 2.39-2.36 (m, 1H), 2.18-2.02 (m, 3H), 1.15 (d, J = 6.9 Hz, 6H). |

### Example 84: (S)-2-(5-(1H-benzo[d][1,2,3]triazol-5-yl)-3-fluoropyridin-2-yl)-N-(4-isopropylphenyl)pyrrolidine-1-carboxamide & (R)-2-(5-(1H-benzo[d][1,2,3]triazol-5-yl)-3-fluoropyridin-2-yl)-N-(4-isopropylphenyl)pyrrolidine-1-carboxamide

### Step 1: tert-butyl 5-(4,4,5,5-tetramethyl-1,3,2-dioxaborolan-2-yl)-1H-benzo[d][1,2,3]triazole-1-carboxylate

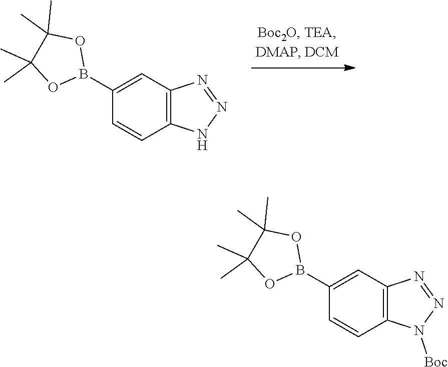 [SMILES: CC1(C)OB(c2ccc3[nH]nnc3c2)OC1(C)C ; CC(C)(C)OC(=O)n1nnc2cc(B3OC(C)(C)C(C)(C)O3)ccc21]

A solution of 5-(4,4,5,5-tetramethyl-1,3,2-dioxaborolan-2-yl)-1H-1,2,3-benzotriazole (400 mg, 1.6 mmol), Boc₂O (534 mg, 2.4 mmol), DMAP (39 mg, 0.3 mmol) and triethylamine (495 mg, 4.8 mmol) in DCM (5 mL) was stirred at room temperature under nitrogen atmosphere. After 2 hours, the resulting mixture was concentrated under reduced pressure. The crude residue was purified by silica gel column chromatography, eluted with PE/EA (1:1) to afford tert-butyl 5-(4,4,5,5-tetramethyl-1,3,2-dioxaborolan-2-yl)-1H-benzo[d][1,2,3]triazole-1-carboxylate (291 mg, 51% yield) as a white solid. LCMS (ESI) [M+H]⁺: 346

### Step 2: tert-butyl 2-(5-chloro-3-fluoropyridin-2-yl)pyrrolidine-1-carboxylate

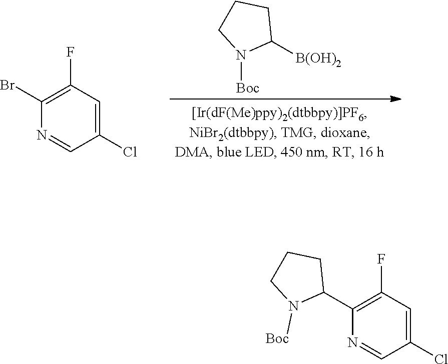 [SMILES: Fc1cc(Cl)cnc1Br ; CC(C)(C)OC(=O)N1CCCC1B(O)O ; CC(C)(C)OC(=O)N1CCCC1c1ncc(Cl)cc1F]

N,N-dimethylacetamide (140 mL) was slowly added into a solution of 2-bromo-5-chloro-3-fluoropyridine (7.0 g, 33.3 mmol), 1-(tert-butoxycarbonyl)pyrrolidin-2-ylboronic acid (10.7 g, 49.9 mmol), [Ir(dF(Me)ppy)₂(dtbbpy)]PF₆ (843 mg, 0.83 mmol), N,N,N′,N′-tetramethylguanidine (11.4 g, 99.8 mmol) and [4,4′-Bis(tert-butyl)-2,2′-bipyridine]nickel dibromide (1.6 g, 3.32 mmol) in dioxane (560 mL) at room temperature under nitrogen atmosphere. The resulting mixture was stirred at room temperature with irradiation at 450 nm blue light for 16 hours. Upon completion, the reaction was quenched with water at room temperature. The resulting mixture was extracted with EtOAc (3×300 mL). The combined organic layers were washed with water (3×200 mL), dried over anhydrous Na₂SO₄, filtered and concentrated under reduced pressure. The crude residue was purified by silica gel column chromatography, eluted with PE/EA (6:1) to afford tert-butyl 2-(5-chloro-3-fluoropyridin-2-yl)pyrrolidine-1-carboxylate (4.8 g, 48% yield) as a light yellow oil. LCMS (ESI) [M+H]⁺: 301

### Step 3: 5-chloro-3-fluoro-2-(pyrrolidin-2-yl)pyridine

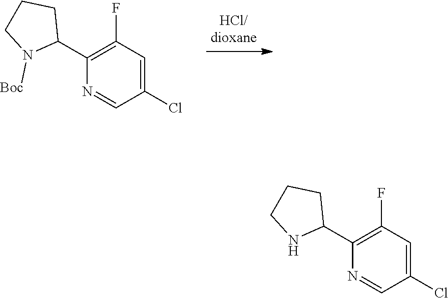 [SMILES: CC(C)(C)OC(=O)N1CCCC1c1ncc(Cl)cc1F ; Fc1cc(Cl)cnc1C1CCCN1]

HCl (50 mL, 200 mmol, 4 M in dioxane) was added slowly into a solution of tert-butyl 2-(5-chloro-3-fluoropyridin-2-yl)pyrrolidine-1-carboxylate (4.8 g, 15.96 mmol) in dioxane (20 mL) and the resulting solution was stirred at room temperature for 1 hour. Upon completion, the resulting mixture was concentrated under reduced pressure to afford 5-chloro-3-fluoro-2-(pyrrolidin-2-yl)pyridine (3.2 g, crude) as a yellow solid, which was used directly in the next step without further purification. LCMS (ESI) [M+H]⁺: 201

### Step 4: 2-(5-chloro-3-fluoropyridin-2-yl)-N-(4-isopropylphenyl)pyrrolidine-1-carboxamide

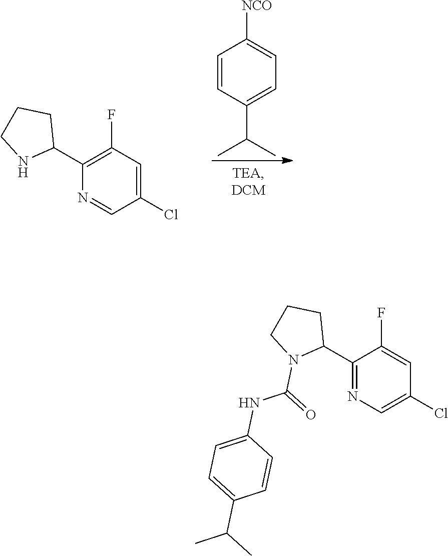

A solution of 5-chloro-3-fluoro-2-(pyrrolidin-2-yl)pyridine (3.3 g, 16.4 mmol) and 1-isocyanato-4-isopropylbenzene (2.7 g, 16.4 mmol), triethylamine (1.7 g, 16.4 mmol) in DCM (20 mL) was stirred at room temperature for 2 hours under nitrogen atmosphere. Upon completion, the resulting mixture was concentrated under reduced pressure. The crude residue was purified by reversed-phase flash chromatography (conditions: column: C18 silica gel; mobile phase A: water (10 mmol/L NH₄HCO₃), Mobile phase B: MeCN; Gradient (B %): 10% to 60% in 15 min; detector, UV 254 nm) to afford 2-(5-chloro-3-fluoropyridin-2-yl)-N-(4-isopropylphenyl)pyrrolidine-1-carboxamide (2.4 g, 40% yield) as a yellow solid. LCMS (ESI) [M+H]⁺: 362

### Step 5: tert-butyl 5-(5-fluoro-6-(1-((4-isopropylphenyl)carbamoyl)pyrrolidin-2-yl)pyridin-3-yl)-1H-benzo[d][1,2,3]triazole-1-carboxylate

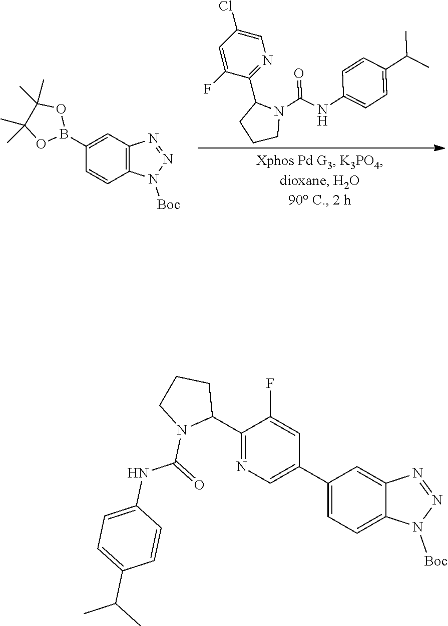 [SMILES: CC(C)(C)OC(=O)n1nnc2cc(B3OC(C)(C)C(C)(C)O3)ccc21 ; CC(C)c1ccc(NC(=O)N2CCCC2c2ncc(Cl)cc2F)cc1 ; CC(C)c1ccc(NC(=O)N2CCCC2c2ncc(-c3ccc4c(c3)nnn4C(=O)OC(C)(C)C)cc2F)cc1]

A solution of tert-butyl 5-(4,4,5,5-tetramethyl-1,3,2-dioxaborolan-2-yl)-1H-benzo[d][1,2,3]triazole-1-carboxylate (150 mg, 0.4 mmol), 2-(5-chloro-3-fluoropyridin-2-yl)-N-(4-isopropylphenyl)pyrrolidine-1-carboxamide (235 mg, 0.6 mmol), Xphos Pd G3 (36 mg, 0.04 mmol) and K₃PO₄ (276 mg, 1.3 mmol) in dioxane (5 mL) and H₂O (1 mL) was stirred at 90° C. overnight under nitrogen atmosphere. Upon completion, the resulting mixture was concentrated under reduced pressure. The crude residue was purified by silica gel column chromatography, eluted with CH₂Cl₂/MeOH (9:1) to afford tert-butyl 5-(5-fluoro-6-(1-((4-isopropylphenyl)carbamoyl)pyrrolidin-2-yl)pyridin-3-yl)-1H-benzo[d][1,2,3]triazole-1-carboxylate (52 mg, 21% yield) as a white solid. LCMS (ESI) [M+H]⁺: 545

### Step 6: 2-(5-(1H-benzo[d][1,2,3]triazol-5-yl)-3-fluoropyridin-2-yl)-N-(4-isopropylphenyl)pyrrolidine-1-carboxamide

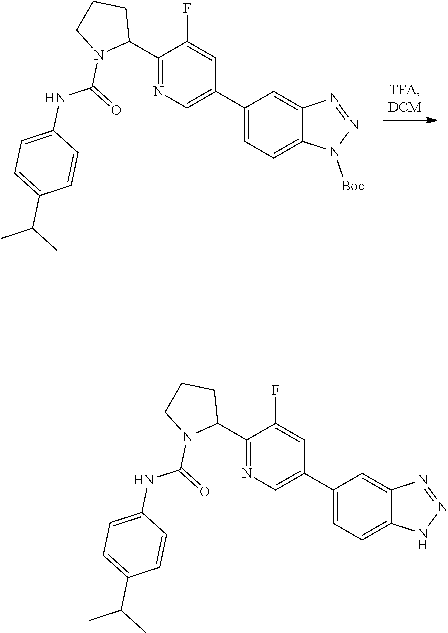 [SMILES: CC(C)c1ccc(NC(=O)N2CCCC2c2ncc(-c3ccc4c(c3)nnn4C(=O)OC(C)(C)C)cc2F)cc1 ; CC(C)c1ccc(NC(=O)N2CCCC2c2ncc(-c3ccc4[nH]nnc4c3)cc2F)cc1]

A solution of tert-butyl 5-(5-fluoro-6-(1-((4-isopropylphenyl)carbamoyl)pyrrolidin-2-yl)pyridin-3-yl)-1H-benzo[d][1,2,3]triazole-1-carboxylate (50 mg, 0.09 mmol) and TFA (1.5 mL) in DCM (5 mL) was stirred at room temperature for 1 hour. Upon completion, the resulting mixture was concentrated under reduced pressure. The crude residue was purified by reversed-phase flash chromatography (conditions: column: C18 silica gel; mobile phase A: water (10 mmol/L NH₄HCO₃), Mobile phase B: MeCN; Gradient (B %): 10% to 70% in 20 min; detector, UV 254 nm) to 2-(5-(1H-benzo[d][1,2,3]triazol-5-yl)-3-fluoropyridin-2-yl)-N-(4-isopropylphenyl)pyrrolidine-1-carboxamide (20 mg, 49% yield) as a white solid. LCMS (ESI) [M+H]⁺: 445

### Step 7: (S)-2-(5-(1H-benzo[d][1,2,3]triazol-5-yl)-3-fluoropyridin-2-yl)-N-(4-isopropylphenyl)pyrrolidine-1-carboxamide & (R)-2-(5-(1H-benzo[d][1,2,3]triazol-5-yl)-3-fluoropyridin-2-yl)-N-(4-isopropylphenyl)pyrrolidine-1-carboxamide

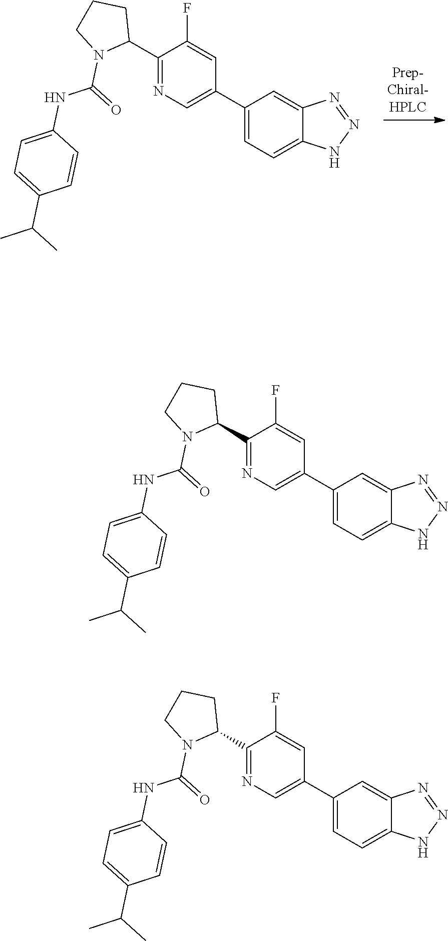 [SMILES: CC(C)c1ccc(NC(=O)N2CCCC2c2ncc(-c3ccc4[nH]nnc4c3)cc2F)cc1 ; CC(C)c1ccc(NC(=O)N2CCC[C@H]2c2ncc(-c3ccc4[nH]nnc4c3)cc2F)cc1 ; CC(C)c1ccc(NC(=O)N2CCC[C@@H]2c2ncc(-c3ccc4[nH]nnc4c3)cc2F)cc1]

2-(5-(1H-benzo[d][1,2,3]triazol-5-yl)-3-fluoropyridin-2-yl)-N-(4-isopropylphenyl)pyrrolidine-1-carboxamide (20 mg, 0.045 mmol) was separated by Prep-Chiral-HPLC (conditions: Column: CHIRALPAK IK 3*25 cm, 5 μm; Mobile Phase A: Hex (0.5% 2M NH₃-MeOH), Mobile Phase B: EtOH; Flow rate: 40 mL/min; Gradient (B %): isocratic 30% B; Wave Length: 220/254 nm; RT1 (min): 12.39; RT2 (min): 17.16) to afford (Compound 84a; Method E, Peak 1, 1.51 min) (S)-2-(5-(1H-benzo[d][1,2,3]triazol-5-yl)-3-fluoropyridin-2-yl)-N-(4-isopropylphenyl)pyrrolidine-1-carboxamide (4.5 mg, 23% yield, 1^{st} peak) as an white solid and (Compound 84b; Method E, Peak 2, 1.74 min) (R)-2-(5-(1H-benzo [d][1,2,3]triazol-5-yl)-3-fluoropyridin-2-yl)-N-(4-isopropylphenyl)pyrrolidine-1-carboxamide (5.3 mg, 23%, 2^{nd} peak) as a white solid.

| Cmpd # | Characterization Data |
|---|---|
| 84a | LCMS (ESI) [M + H]⁺: 445.3 |
| | ¹H NMR (400 MHz, DMSO-d₆) δ 8.77 (s, 1H), 8.30 (bs, 1H), 8.18-8.06 (m, 2H), 8.01 (d, J = 8.4 |
| | Hz, 1H), 7.82 (d, J = 8.8 Hz, 1H), 7.34 (d, J = 8.4 Hz, 2H), 7.04 (d, J = 8.4 Hz, 2H), 5.39-5.31 (m, |
| | 1H), 3.83-3.71 (m, 1H), 3.68-3.58 (m, 1H), 2.84-2.72 (m, 1H), 2.42-2.28 (m, 1H), 2.20-1.80 |
| | (m, 3H), 1.14 (d, J = 7.2 Hz, 6H). |
| 84b | LCMS (ESI) [M + H]⁺: 445.3 |
| | ¹H NMR (400 MHz, DMSO-d₆) δ 8.77 (s, 1H), 8.30 (bs, 1H), 8.17-8.07 (m, 2H), 8.06-7.96 (m, |
| | 1H), 7.83 (d, J = 8.4 Hz, 1H), 7.33 (d, J = 8.4 Hz, 2H), 7.04 (d, J = 8.4 Hz, 2H), 5.41-5.28 (m, 1H), |
| | 3.85-3.71 (m, 1H), 3.69-3.54 (m, 1H), 2.84-2.71 (m, 1H), 2.41-2.29 (m, 1H), 2.20-1.83 (m, |
| | 3H), 1.14 (d, J = 7.2 Hz, 6H). |

### Example 85: (R)-4-(6-(1-((2,2-difluorobenzo[d][1,3]dioxol-5-yl)carbamoyl)pyrrolidin-2-yl)-5-fluoropyridin-3-yl)benzoic acid

### Step 1: methyl 4-(6-chloro-5-fluoropyridin-3-yl)benzoate

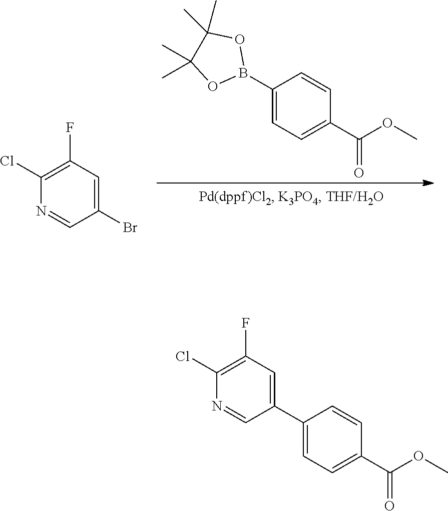

A mixture of methyl 4-(4,4,5,5-tetramethyl-1,3,2-dioxaborolan-2-yl)benzoate (249 g, 950 mmol), 5-bromo-2-chloro-3-fluoropyridine (200 g, 950 mmol) and Pd(dppf)Cl₂ (69.5 g, 95.0 mmol) in THF (2000 mL) was added solution of K₃PO₄ (504 g, 2.38 mol) in H₂O (400 mL). The mixture was stirred at 60° C. for 2 hours under an atmosphere of nitrogen. Upon completion, the resulting mixture was concentrated under reduced pressure. The mixture was extracted with ethyl acetate (2×500 mL). The combined organic layers were washed with H₂O (2×300 mL), dried over anhydrous Na₂SO₄, filtered and concentrated under reduced pressure. The crude residue was purified by silica gel column chromatography, eluted with petroleum ether/ethyl acetate (8:1) to afford methyl 4-(6-chloro-5-fluoropyridin-3-yl)benzoate (190 g, 75% yield) as a white solid. LCMS (ESI) [M+H]⁺: 266

### Step 2: tert-butyl (2R,4S)-2-(3-fluoro-5-(4-(methoxycarbonyl)phenyl)pyridin-2-yl)-4-hydroxypyrrolidine-1-carboxylate

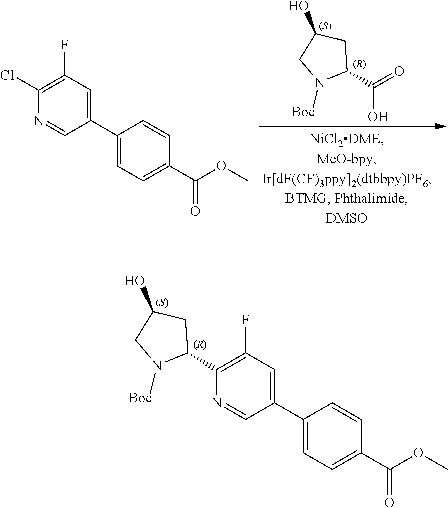

A solution of methyl 4-(6-chloro-5-fluoropyridin-3-yl)benzoate (160 g, 602 mmol), (2R,4S)-1-(tert-butoxycarbonyl)-4-hydroxypyrrolidine-2-carboxylic acid (153 g, 662 mmol), 2,3-dihydro-1H-isoindole-1,3-dione (88.6 g, 602 mmol), 4,4′-dimethoxy-2,2′-bipyridine (15.6 g, 72.3 mmol), dichloronickel;1,2-dimethoxyethane (13.2 g, 60.2 mmol), Ir[dF(CF3)ppy]2(dtbpy))PF6 (13.5 g, 12.0 mmol), and N″-tert-butyl-N,N,N′,N′-tetramethylguanidine (155 g, 903 mmol) in DMSO (3000 mL) was stirred at room temperature, irradiated with 450 nm blue LEDs under an atmosphere of nitrogen for 1 day. Upon completion, the reaction mixture was diluted with H₂O (1000 mL). The resulting mixture was extracted with ethyl acetate (3×5000 mL). The combined organic layers were washed with H₂O (5×3000 mL), dried over anhydrous Na₂SO₄, filtered and concentrated under reduced pressure. The crude residue was purified by silica gel column chromatography, eluted with petroleum ether/ethyl acetate (2:1) to afford tert-butyl (2R,4S)-2-(3-fluoro-5-(4-(methoxycarbonyl)phenyl)pyridin-2-yl)-4-hydroxypyrrolidine-1-carboxylate (45.8 g, 18%) as a yellow solid. LCMS (ESI) [M+H]⁺: 417.

See Rajender Nallagonda ACS Catal. 2024, 14, 13439-13450 for precedent for relevant stereoselective synthetic procedures.

### Step 3: tert-butyl (2R,4S)-2-(3-fluoro-5-(4-(methoxycarbonyl)phenyl)pyridin-2-yl)-4-((phenoxycarbonothioyl)oxy)pyrrolidine-1-carboxylate

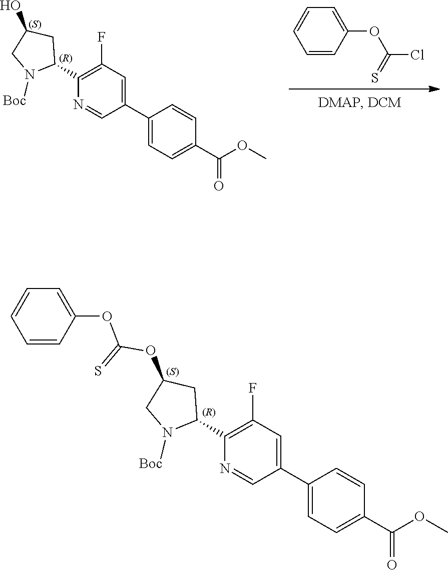

A mixture of tert-butyl (2R,4S)-2-(3-fluoro-5-(4-(methoxycarbonyl)phenyl)pyridin-2-yl)-4-hydroxypyrrolidine-1-carboxylate (20 g, 48 mmol), phenyl (41.4 g, 240 mmol) and DMAP (11.7 g, 96.0 mmol) in ACN (400 mL) was stirred at room temperature for 2 hours under an atmosphere of nitrogen. Upon completion, the resulting mixture was concentrated under reduced pressure to afford crude tert-butyl (2R,4S)-2-(3-fluoro-5-(4-(methoxycarbonyl)phenyl)pyridin-2-yl)-4-((phenoxycarbonothioyl)oxy)pyrrolidine-1-carboxylate (30 g, quant), which was used in the next step directly without further purification. LCMS (ESI) [M+H]⁺: 553.

### Step 4: tert-butyl (R)-2-(3-fluoro-5-(4-(methoxycarbonyl)phenyl)pyridin-2-yl)pyrrolidine-1-carboxylate

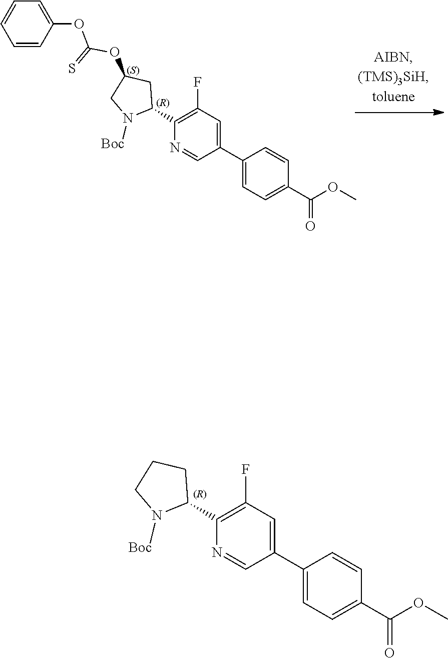

A solution of tert-butyl (2R,4S)-2-(3-fluoro-5-(4-(methoxycarbonyl)phenyl)pyridin-2-yl)-4-((phenoxycarbonothioyl)oxy)pyrrolidine-1-carboxylate (30.0 g, 54.3 mmol), 1,1,1,3,3,3-hexamethyl-2-(trimethylsilyl)trisilane (33.8 g, 135 mmol) and AIBN (17.8 g, 109 mmol) in toluene (400 mL) was stirred at 90° C. for 1 hour. Upon completion, the resulting mixture was concentrated under reduced pressure. The crude residue was directly purified by silica gel column chromatography, eluted with petroleum ether/ethyl acetate (3:1) to afford tert-butyl (R)-2-(3-fluoro-5-(4-(methoxycarbonyl)phenyl)pyridin-2-yl)pyrrolidine-1-carboxylate (12.8 g, 59%) as a colorless oil. LCMS (ESI) [M+H]⁺: 401.

### Step 5: methyl (R)-4-(5-fluoro-6-(pyrrolidin-2-yl)pyridin-3-yl)benzoate

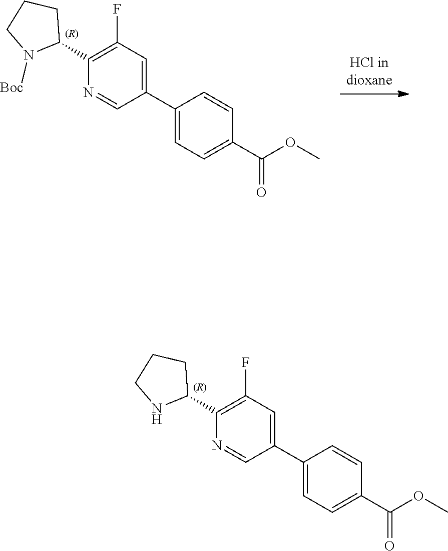

Hydrochloric acid (40 mL, 160 mmol, 4 M in 1,4-dioxane) was added into a solution of tert-butyl (R)-2-(3-fluoro-5-(4-(methoxycarbonyl)phenyl)pyridin-2-yl)pyrrolidine-1-carboxylate (12.8 g, 32.0 mmol) in 1,4-dioxane (40 mL) at room temperature. After 2 hours, the mixture was concentrated under reduced pressure to afford crude methyl (R)-4-(5-fluoro-6-(pyrrolidin-2-yl)pyridin-3-yl)benzoate (9.2 g, quant) as a colorless oil, which was used in the next step without further purification. LCMS (ESI) [M+H]⁺: 301.3.

### Step 6: (R)-4-(5-fluoro-6-(pyrrolidin-2-yl)pyridin-3-yl)benzoic acid

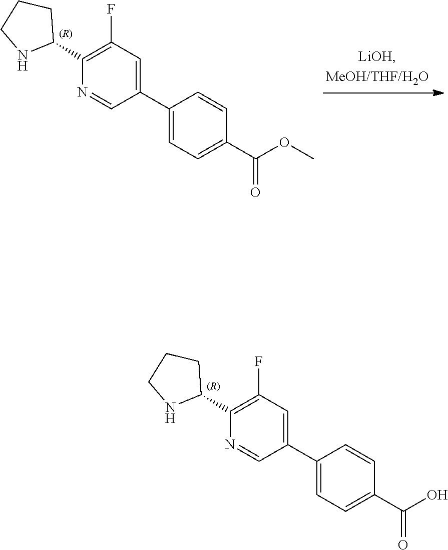

A solution of methyl (R)-4-(5-fluoro-6-(pyrrolidin-2-yl)pyridin-3-yl)benzoate (9.2 g, 30.6 mmol) and lithium hydroxide monohydrate (5.15 g, 123 mmol) in THF (500 mL), MeOH (200 mL) and H₂O (100 mL) was stirred at 60° C. for 2 hours. Upon completion, the resulting mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 30 min; detector: UV 254 nm) to afford (R)-4-(5-fluoro-6-(pyrrolidin-2-yl)pyridin-3-yl)benzoic acid (6.1 g, 69%) as a white solid. LCMS (ESI) [M+H]⁺: 287.0.

### Step 7: (R)-4-(6-(1-((2,2-difluorobenzo[d][1,3]dioxol-5-yl)carbamoyl)pyrrolidin-2-yl)-5-fluoropyridin-3-yl)benzoic acid

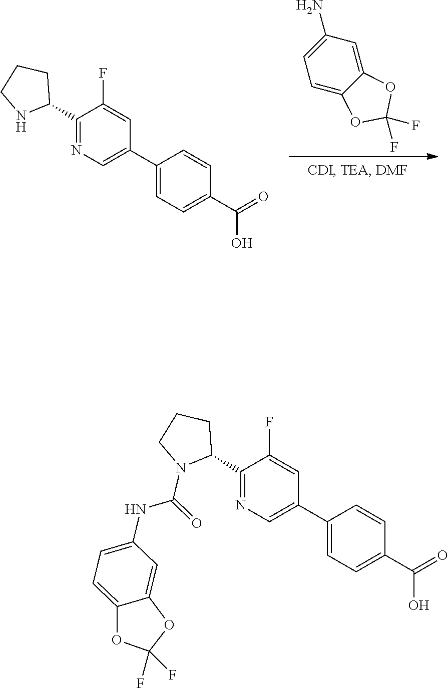 [SMILES: O=C(O)c1ccc(-c2cnc([C@H]3CCCN3)c(F)c2)cc1 ; Nc1ccc2c(c1)OC(F)(F)O2 ; O=C(O)c1ccc(-c2cnc([C@H]3CCCN3C(=O)Nc3ccc4c(c3)OC(F)(F)O4)c(F)c2)cc1]

A solution of 2,2-difluoro-1,3-benzodioxol-5-amine (363 mg, 2.10 mmol), triethylamine (212 mg, 2.10 mmol) in DMF (3 mL) was treated with CDI (340 mg, 2.10 mmol) at room temperature. After 1 minute, (R)-4-(5-fluoro-6-(pyrrolidin-2-yl)pyridin-3-yl)benzoic acid (200 mg, 0.70 mmol) was added. The resulting mixture was stirred at 60° C. for 2 hours. Upon completion, the reaction mixture was directly purified by reversed-phase flash chromatography (conditions: column: C18 silica gel; mobile phase A: water (10 mmol/L FA), Mobile phase B: MeCN; Gradient (B %): 10% to 70% in 22 min; detector, UV 254 nm) to yield (Compound 85; Method AJ, Peak 1, 1.18 min—analyzed against Compound 198) (R)-4-(6-(1-((2,2-difluorobenzo[d][1,3]dioxol-5-yl)carbamoyl)pyrrolidin-2-yl)-5-fluoropyridin-3-yl)benzoic acid (75.9 mg, 22% yield) as a white solid.

| Cmpd # | Characterization Data |
|---|---|
| 85 | LCMS (ESI) [M + H]⁺: 486.2 |
| | ¹H NMR (400 MHz, DMSO-d₆) δ 13.04 (bs, 1H), 8.74 (t, J = 1.6 Hz, 1H), 8.48 (s, 1H), 8.08 (dd, J = |
| | 11.5, 1.9 Hz, 1H), 8.07-8.00 (m, 2H), 7.93-7.86 (m, 2H), 7.58 (d, J = 2.0 Hz, 1H), 7.26-7.14 |
| | (m, 2H), 5.35 (dd, J = 8.2, 4.1 Hz, 1H), 3.76 (td, J = 8.6, 8.1, 5.1 Hz, 1H), 3.63 (dt, J = 9.5, 7.1 Hz, |
| | 1H), 2.42-2.29 (m, 1H), 2.17-2.06 (m, 1H), 2.06-1.98 (m, 1H), 1.91 (dt, J = 11.4, 5.8 Hz, 1H). |

### Example 86: (S)-4-(1′-((4-isopropylphenyl)carbamoyl)-5,6-dihydrospiro[cyclopenta[b]pyridine-7,2′-pyrrolidin]-3-yl)benzoic acid & (R)-4-(1′-((4-isopropylphenyl)carbamoyl)-5,6-dihydrospiro[cyclopenta[b]pyridine-7,2′-pyrrolidin]-3-yl)benzoic acid

### Step 1: 2-bromo-3-(bromomethyl)-5-chloropyridine

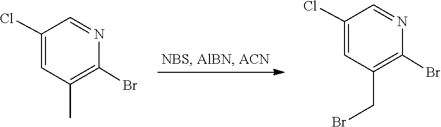

A mixture of 2-bromo-5-chloro-3-methylpyridine (1.00 g, 4.80 mmol), NBS (948 mg, 5.30 mmol), and AIBN (79.5 mg, 0.484 mmol) in acetonitrile (10.0 mL) was heated to 80° C. and stirred for 1 hour. Upon completion, the mixture was cooled to room temperature and concentrated under reduced pressure. The crude residue was directly purified by silica gel column chromatography, eluted with petroleum ether/ethyl acetate (3:1), to afford 2-bromo-3-(bromomethyl)-5-chloropyridine (800 mg, 58%) as a white oil. LCMS (ESI) [M+H]⁺: 284.8

### Step 2: 2-bromo-5-chloro-3-(2-(3,4-dihydro-2H-pyrrol-5-yl)ethyl)pyridine

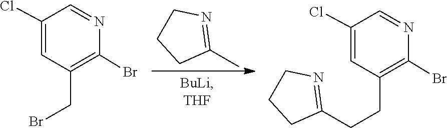

To a solution of 5-methyl-3,4-dihydro-2H-pyrrole (233 mg, 2.80 mmol) in THF (5.0 mL) at −78° C. was added dropwise n-butyllithium (3.0 mL, 7.5 mmol, 2.5 M in n-hexane) under nitrogen atmosphere. After 30 minutes, 2-bromo-3-(bromomethyl)-5-chloropyridine (800 mg, 2.80 mmol) was added portion-wise. After stirring for an additional 30 minutes, the solution was warmed to room temperature. After 1 hour, the reaction mixture was cooled in an ice bath and quenched by the dropwise addition of water (3.0 mL). The resultant mixture was extracted with ethyl acetate (3×10 mL). The combined organic layers were washed with water (10 mL), dried over anhydrous Na₂SO₄, filtered and concentrated under reduced pressure to afford crude 2-bromo-5-chloro-3-[2-(4,5-dihydro-3H-pyrrol-2-yl)ethyl]pyridine (600 mg, 74%) as a brown oil, which was used directly in the next step without further purification. LCMS (ESI) [M+H]⁺: 287.5

### Step 3: 3-chloro-5,6-dihydrospiro[cyclopenta[b]pyridine-7,2′-pyrrolidine]

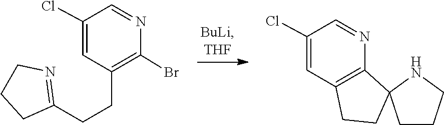 [SMILES: Clc1cnc(Br)c(CCC2=NCCC2)c1 ; Clc1cnc2c(c1)CCC21CCCN1]

To a stirred solution of 2-bromo-5-chloro-3-[2-(4,5-dihydro-3H-pyrrol-2-yl)ethyl]pyridine (600 mg, 2.09 mmol) in THF (5.0 mL) at −78° C. was added n-butyllithium (2.00 mL, 5.00 mmol, 2.5 M in n-hexane) under nitrogen atmosphere. The mixture was warmed to room temperature and stirred for 1 hour. Upon completion, the reaction was cooled in an ice bath and quenched by the dropwise addition of water (3.0 mL). The mixture was extracted with ethyl acetate (3×10 mL). The combined organic layers were washed with water (10 mL), dried over anhydrous Na₂SO₄, filtered and concentrated under reduced pressure to afford crude 3-chloro-5,6-dihydrospiro[cyclopenta[b]pyridine-7,2′-pyrrolidine](400 mg, 91%) as a brown oil, which was used directly in the next step without further purification. LCMS (ESI) [M+H]⁺: 209.0

### Step 4: 3-chloro-N-(4-isopropylphenyl)-5,6-dihydrospiro[cyclopenta[b]pyridine-7,2′-pyrrolidine]-1′-carboxamide

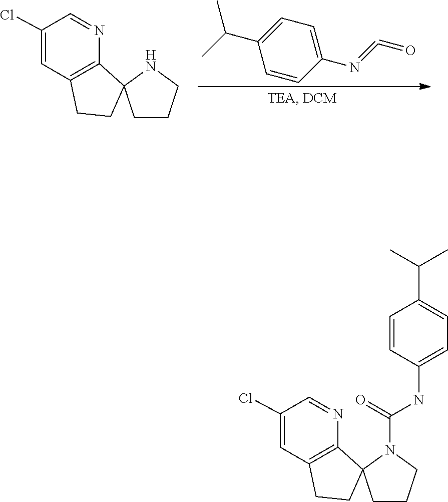

A solution of 3-chloro-5,6-dihydrospiro[cyclopenta[b]pyridine-7,2′-pyrrolidine](400 mg, 1.90 mmol), 1-isocyanato-4-isopropylbenzene (371 mg, 2.30 mmol) and TEA (388 mg, 3.80 mmol) in DCM (5.0 mL) was stirred at room temperature for 1 hour. Upon completion, the mixture was concentrated under reduced pressure. The crude residue was directly purified by reversed-phase flash chromatography (conditions: column: C18 silica gel; mobile phase A: water (10 mmol/L formic acid), mobile phase B: acetonitrile; gradient (B %): 0% to 50% over 20 min; detector: UV 254 nm) to afford 3-chloro-N-(4-isopropylphenyl)-5,6-dihydrospiro[cyclopenta[b]pyridine-7,2′-pyrrolidine]-1′-carboxamide (120 mg, 17%) as a brown oil. LCMS (ESI) [M+H]⁺: 370.1

### Step 5: methyl 4-(1′-((4-isopropylphenyl)carbamoyl)-5,6-dihydrospiro[cyclopenta[b]pyridine-7,2′-pyrrolidin]-3-yl)benzoate

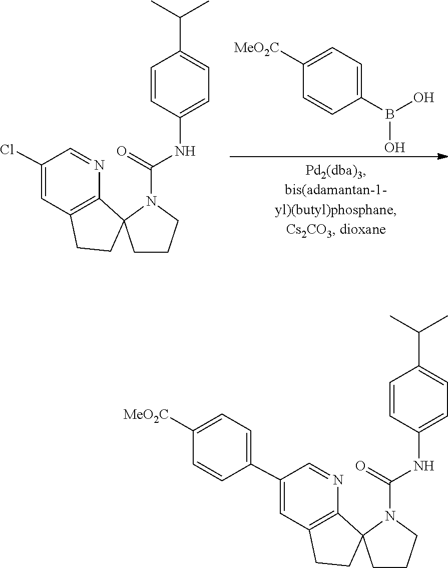 [SMILES: CC(C)c1ccc(NC(=O)N2CCCC23CCc2cc(Cl)cnc23)cc1 ; COC(=O)c1ccc(B(O)O)cc1 ; COC(=O)c1ccc(-c2cnc3c(c2)CCC32CCCN2C(=O)Nc2ccc(C(C)C)cc2)cc1]

A solution of 3-chloro-N-(4-isopropylphenyl)-5,6-dihydrospiro[cyclopenta[b]pyridine-7,2′-pyrrolidine]-1′-carboxamide (110 mg, 0.297 mmol), 4-(methoxycarbonyl)phenylboronic acid (64 mg, 0.356 mmol), Pd₂(dba)₃ (27.2 mg, 29.7 mmol), bis(adamantan-1-yl)(butyl)phosphane (32.0 mg, 89.2 mmol) and Cs₂CO₃ (194 mg, 0.595 mmol) in dioxane (2.0 mL) was heated to 100° C. and stirred for 2 hours under nitrogen atmosphere. Upon completion, the mixture was concentrated under reduced pressure. The crude residue was directly purified by silica gel column chromatography, eluted with petroleum ether/EA (2/1), to afford methyl 4-(1′-((4-isopropylphenyl)carbamoyl)-5,6-dihydrospiro[cyclopenta[b]pyridine-7,2′-pyrrolidin]-3-yl)benzoate (50 mg, 36%) as a brown oil. LCMS (ESI) [M+H]⁺: 470.2

### Step 6: 4-(1′-((4-isopropylphenyl)carbamoyl)-5,6-dihydrospiro[cyclopenta[b]pyridine-7,2′-pyrrolidin]-3-yl)benzoic acid

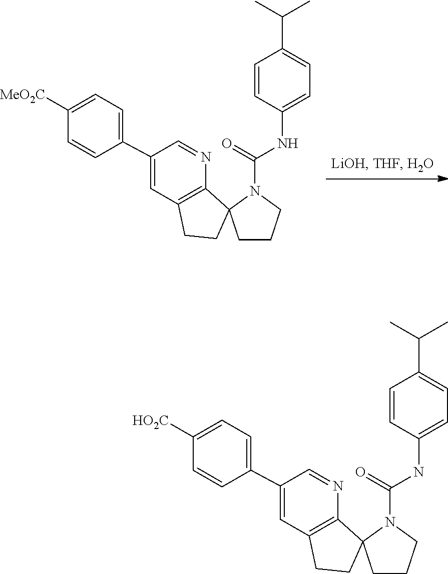 [SMILES: COC(=O)c1ccc(-c2cnc3c(c2)CCC32CCCN2C(=O)Nc2ccc(C(C)C)cc2)cc1 ; CC(C)c1ccc(NC(=O)N2CCCC23CCc2cc(-c4ccc(C(=O)O)cc4)cnc23)cc1]

A solution of 4-(1′-((4-isopropylphenyl)carbamoyl)-5,6-dihydrospiro[cyclopenta[b]pyridine-7,2′-pyrrolidin]-3-yl)benzoate (50.0 mg, 0.110 mmol) and LiOH monohydrate (8.40 mg, 0.200 mmol) in water (1.0 mL) and THF (1.0 mL) was stirred for 1 hour at room temperature. Upon completion, the mixture was acidified to pH ˜2 with HCl (12 N). The resultant mixture was extracted with ethyl acetate (3×10 mL). The combined organic layers were washed with water (10 mL), dried over anhydrous Na₂SO₄, filtered and concentrated under reduced pressure. The crude residue was purified by reversed-phase flash chromatography (conditions: column: C18 silica gel; mobile phase A: water (10 mmol/L formic acid), mobile phase B: acetonitrile; gradient (B %): 0% to 100% over 24 min; detector: UV 254 nm) to afford 4-(1′-((4-isopropylphenyl)carbamoyl)-5,6-dihydrospiro[cyclopenta[b]pyridine-7,2′-pyrrolidin]-3-yl)benzoic acid (30 mg, 62%) as a white solid. LCMS (ESI) [M+H]⁺: 456.2

### Step 7: (S)-4-(1′-((4-isopropylphenyl)carbamoyl)-5,6-dihydrospiro[cyclopenta[b]pyridine-7,2′-pyrrolidin]-3-yl)benzoic acid & (R)-4-(1′-((4-isopropylphenyl)carbamoyl)-5,6-dihydrospiro[cyclopenta[b]pyridine-7,2′-pyrrolidin]-3-yl)benzoic acid

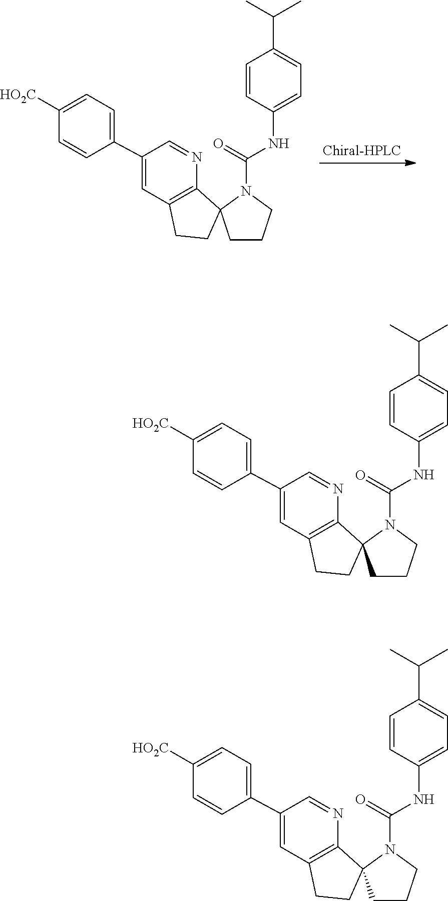 [SMILES: CC(C)c1ccc(NC(=O)N2CCCC23CCc2cc(-c4ccc(C(=O)O)cc4)cnc23)cc1 ; CC(C)c1ccc(NC(=O)N2CCC[C@]23CCc2cc(-c4ccc(C(=O)O)cc4)cnc23)cc1 ; CC(C)c1ccc(NC(=O)N2CCC[C@@]23CCc2cc(-c4ccc(C(=O)O)cc4)cnc23)cc1]

4-(1′-((4-Isopropylphenyl)carbamoyl)-5,6-dihydrospiro[cyclopenta[b]pyridine-7,2′-pyrrolidin]-3-yl)benzoic acid (30 mg) was resolved by preparative chiral-HPLC (conditions: column: CHIRAL ART Cellulose-SC, 3*25 cm, 5 μm; mobile phase A: hexanes (0.1% formic acid), mobile phase B: ethanol; flow rate: 40 mL/min; gradient (B %): isocratic 25% B; detector: 220/254 nm; RT1 (min): 6.1; RT2 (min): 7.8) to afford (Compound 86a; Method W, Peak 1, 1.17 min) (S)-4-(1′-((4-isopropylphenyl)carbamoyl)-5,6-dihydrospiro[cyclopenta[b]pyridine-7,2′-pyrrolidin]-3-yl)benzoic acid (1^{st} peak, 9.9 mg, 33%) and (Compound 86b; Method W, Peak 2, 2.06 min) (R)-4-(1′-((4-isopropylphenyl)carbamoyl)-5,6-dihydrospiro[cyclopenta[b]pyridine-7,2′-pyrrolidin]-3-yl)benzoic acid (2^{nd} peak, 13.2 mg, 44%).

| Cmpd # | Characterization Data |
|---|---|
| 86a | LCMS (ESI) [M + H]⁺: 456.3 |
| | ¹H NMR (400 MHz, methanol-d₄) δ 8.57 (d, J = 2.0 Hz, 1H), 8.11 (d, J = 8.4 Hz, 2H), 7.92 (d, J = 2.0 |
| | Hz, 1H), 7.74 (d, J = 8.4 Hz, 2H), 7.19 (d, J = 8.6 Hz, 2H), 7.06 (d, J = 8.6 Hz, 2H), 3.86-3.72 (m, |
| | 2H), 3.21-3.07 (m, 1H), 3.02-2.91 (m, 1H), 2.85-2.74 (m, 2H), 2.29-2.06 (m, 5H), 1.17 (d, J = |
| | 6.9 Hz, 6H). |
| 86b | LCMS (ESI) [M + H]⁺: 456.2 |
| | ¹H NMR (400 MHz, methanol-d₄) δ 8.57 (d, J = 2.1 Hz, 1H), 8.11 (d, J = 8.3 Hz, 2H), 7.92 (d, J = 2.0 |
| | Hz, 1H), 7.74 (d, J = 8.4 Hz, 2H), 7.19 (d, J = 8.5 Hz, 2H), 7.06 (d, J = 8.5 Hz, 2H), 3.87-3.70 (m, |
| | 2H), 3.22-3.07 (m, 1H), 3.04-2.90 (m, 1H), 2.87-2.71 (m, 2H), 2.32-2.02 (m, 5H), 1.17 (d, J = |
| | 6.8 Hz, 6H). |

### Example 89: 4-(5-fluoro-6-((2S,3S)-1-((4-isopropylphenyl)carbamoyl)-3-methylpyrrolidin-2-yl)pyridin-3-yl)benzoic acid and 4-(5-fluoro-6-((2R,3R)-1-((4-isopropylphenyl)carbamoyl)-3-methylpyrrolidin-2-yl)pyridin-3-yl)benzoic acid

### Step 1: tert-butyl 2-(5-(4-(tert-butoxycarbonyl)phenyl)-3-fluoropyridin-2-yl)-3-methylpyrrolidine-1-carboxylate

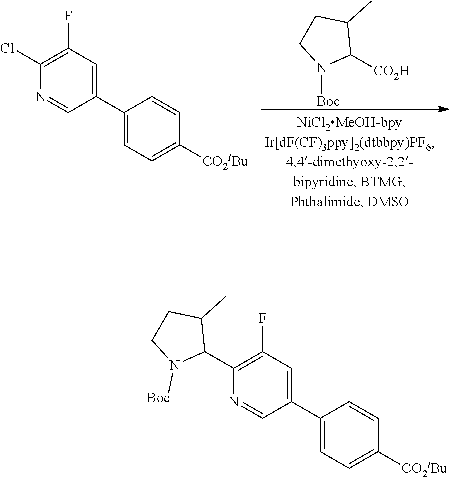 [SMILES: CC(C)(C)OC(=O)c1ccc(-c2cnc(Cl)c(F)c2)cc1 ; CC1CCN(C(=O)OC(C)(C)C)C1C(=O)O ; CC1CCN(C(=O)OC(C)(C)C)C1c1ncc(-c2ccc(C(=O)OC(C)(C)C)cc2)cc1F]

A solution of 1-(tert-butoxycarbonyl)-3-methylpyrrolidine-2-carboxylic acid (687 mg, 3.00 mmol), tert-butyl 4-(6-chloro-5-fluoropyridin-3-yl)benzoate (615 mg, 2.00 mmol), dichloronickel;1,2-dimethoxyethane (44 mg, 0.200 mmol), 4,4′-dimethoxy-2,2′-bipyridine (52 mg, 0.241 mmol), N″-tert-butyl-N,N,N′,N′-tetramethylguanidine (513 mg, 3.00 mmol), Ir[dF(CF₃)ppy]₂(dtbpy))PF₆ (45.0 mg, 40.0 μmol) and 2,3-dihydro-1H-isoindole-1,3-dione (294 mg, 2.00 mmol) in DMSO (10.0 mL) was stirred overnight at room temperature under a nitrogen atmosphere under 450 nM blue LED irradiation. Upon completion, the mixture was diluted with water and extracted with ethyl acetate (3×15 mL). The combined organic layers were washed with brine, dried over anhydrous Na₂SO₄, filtered and concentrated under reduced pressure. The crude residue was purified by reversed-phase flash chromatography (conditions: column, C18 silica gel; mobile phase A: water (0.1% formic acid), mobile phase B: acetonitrile; gradient (B %): 0% to 100% over 25 min; detector: UV 254 nm) to afford tert-butyl 2-(5-(4-(tert-butoxycarbonyl)phenyl)-3-fluoropyridin-2-yl)-3-methylpyrrolidine-1-carboxylate (150 mg, 16%) as an off-white solid. LCMS [M+H]⁺: 457.0

### Step 2: 4-(5-fluoro-6-(3-methylpyrrolidin-2-yl)pyridin-3-yl)benzoic acid

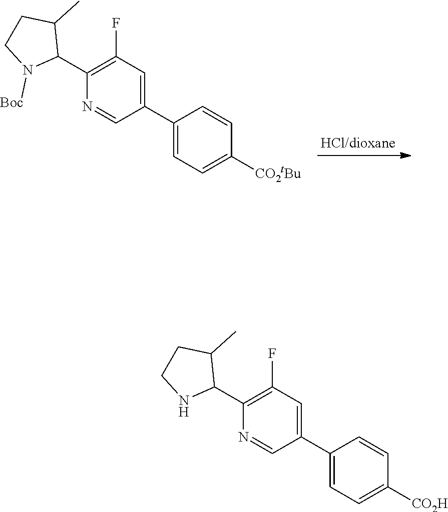 [SMILES: CC1CCN(C(=O)OC(C)(C)C)C1c1ncc(-c2ccc(C(=O)OC(C)(C)C)cc2)cc1F ; CC1CCNC1c1ncc(-c2ccc(C(=O)O)cc2)cc1F]

To a mixture of tert-butyl 2-(5-(4-(tert-butoxycarbonyl)phenyl)-3-fluoropyridin-2-yl)-3-methylpyrrolidine-1-carboxylate (145 mg, 0.318 mmol) in dioxane (1.0 mL) at 0° C. was added HCl (5.0 mL, 20 mmol, 4 M in 1,4-dioxane) dropwise. The mixture was warmed to room temperature and stirred overnight. Upon completion, the resultant mixture was concentrated under reduced pressure to afford 4-(5-fluoro-6-(3-methylpyrrolidin-2-yl)pyridin-3-yl)benzoic acid (98 mg, crude) as an off-white solid, which was used directly in the next step without further purification. LCMS [M+H]⁺: 301.0

### Step 3: 4-(5-fluoro-6-(1-((4-isopropylphenyl)carbamoyl)-3-methylpyrrolidin-2-yl)pyridin-3-yl)benzoic acid

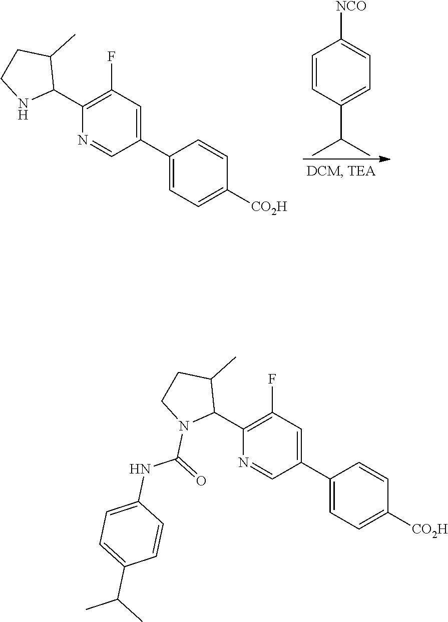

To a mixture of 4-(5-fluoro-6-(3-methylpyrrolidin-2-yl)pyridin-3-yl)benzoic acid (96.0 mg, 0.320 mmol) and TEA (97 mg, 0.960 mmol) in DCM (5.0 mL) at 0° C. was added 1-isocyanato-4-isopropylbenzene (52 mg, 0.323 mmol) dropwise. The resultant mixture was warmed to room temperature and stirred for 1 hour. Upon completion, the mixture was concentrated under reduced pressure. The crude residue was directly purified by reversed-phase flash chromatography (conditions: column: C18 silica gel; mobile phase A: water (0.1% formic acid), mobile phase B: acetonitrile; gradient (B %): 0% to 100% over 25 min; detector: UV 254 nm) to afford 4-(5-fluoro-6-(1-((4-isopropylphenyl)carbamoyl)-3-methylpyrrolidin-2-yl)pyridin-3-yl)benzoic acid (100 mg, 68%) as a white solid. LCMS [M+H]⁺: 462.2

### Step 4: 4-(5-fluoro-6-((2S,3S)-1-((4-isopropylphenyl)carbamoyl)-3-methylpyrrolidin-2-yl)pyridin-3-yl)benzoic acid and 4-(5-fluoro-6-((2R,3R)-1-((4-isopropylphenyl)carbamoyl)-3-methylpyrrolidin-2-yl)pyridin-3-yl)benzoic acid

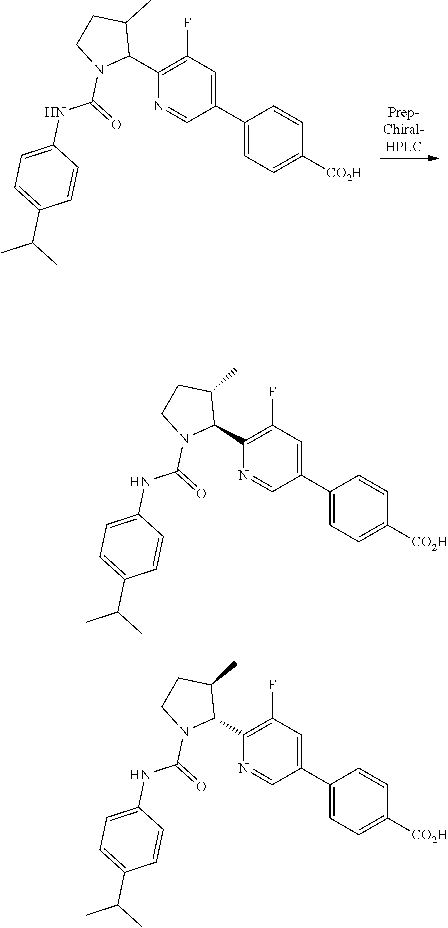 [SMILES: CC(C)c1ccc(NC(=O)N2CCC(C)C2c2ncc(-c3ccc(C(=O)O)cc3)cc2F)cc1 ; CC(C)c1ccc(NC(=O)N2CC[C@H](C)[C@H]2c2ncc(-c3ccc(C(=O)O)cc3)cc2F)cc1 ; CC(C)c1ccc(NC(=O)N2CC[C@@H](C)[C@@H]2c2ncc(-c3ccc(C(=O)O)cc3)cc2F)cc1]

4-(5-Fluoro-6-(1-((4-isopropylphenyl)carbamoyl)-3-methylpyrrolidin-2-yl)pyridin-3-yl)benzoic acid (100 mg) was resolved by preparative chiral HPLC (conditions: column: CHIRAL ART Cellulose-SC, 2*25 cm, 5 μm; mobile phase A: hexane (0.1% formic acid); mobile phase B: ethanol; flow rate: 20 mL/min; gradient (B %): isocratic 30% B; detector: 220/254 nm; RT1 (min): 5.6; RT2 (min): 7.6) to afford (Compound 89a; Method AA, Peak 1, 0.85 min) first peak, 4-(5-fluoro-6-((2S,3S)-1-((4-isopropylphenyl)carbamoyl)-3-methylpyrrolidin-2-yl)pyridin-3-yl)benzoic acid (38.8 mg, 39%), as a white solid and (Compound 89b; Method AA, Peak 2, 1.25 min) second peak, 4-(5-fluoro-6-((2R,3R)-1-((4-isopropylphenyl)carbamoyl)-3-methylpyrrolidin-2-yl)pyridin-3-yl)benzoic acid (38.9 mg, 39%), as a white solid.

| Cmpd # | Characterization Data |
|---|---|
| 89a | LCMS (ESI) [M + H]⁺: 462.2 |
| | ¹H NMR (400 MHz, DMSO-d₆) δ 8.74 (s, 1H), 8.15 (s, 1H), 8.07-8.02 (m, 3H), 7.89 (d, J = 8.0 Hz, |
| | 2H), 7.31 (d, J = 8.0 Hz, 2H), 7.03 (d, J = 8.4 Hz, 2H), 4.84 (d, J = 6.0 Hz, 1H), 3.73 (t, J = 6.2 Hz, |
| | 2H), 2.80-2.74 (m, 1H), 2.35-2.30 (m, 1H), 2.29-2.18 (m, 1H), 1.76-1.67 (m, 1H), 1.13 (d, J = |
| | 6.8 Hz, 9H). |
| 89b | LCMS (ESI) [M + H]⁺: 462.2 |
| | ¹H NMR (400 MHz, DMSO-d₆) δ 8.74 (s, 1H), 8.14 (s, 1H), 8.07-8.02 (m, 3H), 7.89 (d, J = 8.4 Hz, |
| | 2H), 7.31 (d, J = 8.4 Hz, 2H), 7.03 (d, J = 8.4 Hz, 2H), 4.83 (d, J = 6.0 Hz, 1H), 3.73 (t, J = 6.4 Hz, |
| | 2H), 2.80-2.73 (m, 1H), 2.35-2.30 (m, 1H), 2.29-2.18 (m, 1H), 1.74-1.69 (m, 1H), 1.13 (d, J = |
| | 6.8 Hz, 9H). |

### Example 90: (S)-4-(5-fluoro-6-(1-((5-trifluoromethyl)pyridin-2-yl)carbamoyl)pyrrolidin-2-yl)pyridin-3-yl)benzoic acid and (R)-4-(5-fluoro-6-(1-((5-(trifluoromethyl)pyridin-2-yl)carbamoyl)pyrrolidin-2-yl)pyridin-3-yl)benzoic acid

### Step 1: 4-(5-fluoro-6-(1-((5-(trifluoromethyl)pyridin-2-yl)carbamoyl)pyrrolidin-2-yl)pyridin-3-yl)benzoic acid

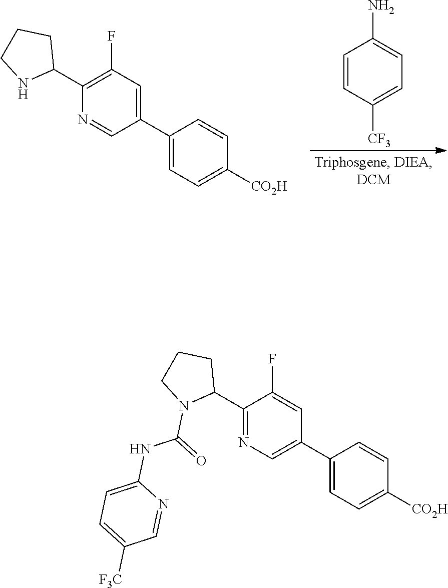 [SMILES: O=C(O)c1ccc(-c2cnc(C3CCCN3)c(F)c2)cc1 ; Nc1ccc(C(F)(F)F)cc1 ; O=C(O)c1ccc(-c2cnc(C3CCCN3C(=O)Nc3ccc(C(F)(F)F)cn3)c(F)c2)cc1]

To a mixture of 6-(trifluoromethyl)pyridin-3-amine (57 mg, 0.352 mmol) and DIEA (3.0 mL) in DCM at 0° C. was added triphosgene (41 mg, 0.139 mmol) dropwise. After 30 minutes, the solution was warmed to room temperature and 4-(5-fluoro-6-(pyrrolidin-2-yl)pyridin-3-yl)benzoic acid (100 mg, 0.350 mmol) was added. After stirring for an additional 2 hours, the mixture was cooled to 0° C. and quenched with ice water. The resultant mixture was extracted with ethyl acetate (3×15 mL). The combined organic layers were dried over anhydrous Na₂SO₄, filtered and concentrated under reduced pressure. The crude residue was purified by reversed-phase flash chromatography (conditions: column: C18 silica gel; mobile phase A: water (0.1% formic acid), mobile phase B: acetonitrile; gradient: 0% to 100% over 25 min; detector: UV 254 nm) to afford 4-(5-fluoro-6-(1-((5-(trifluoromethyl)pyridin-2-yl)carbamoyl)pyrrolidin-2-yl)pyridin-3-yl)benzoic acid (40 mg, 24%) as a white solid. LCMS [M+H]⁺: 475.1

### Step 2: (S)-4-(5-fluoro-6-(1-((5-(trifluoromethyl)pyridin-2-yl)carbamoyl)pyrrolidin-2-yl)pyridin-3-yl)benzoic acid and (R)-4-(5-fluoro-6-(1-((5-(trifluoromethyl)pyridin-2-yl)carbamoyl)pyrrolidin-2-yl)pyridin-3-yl)benzoic acid

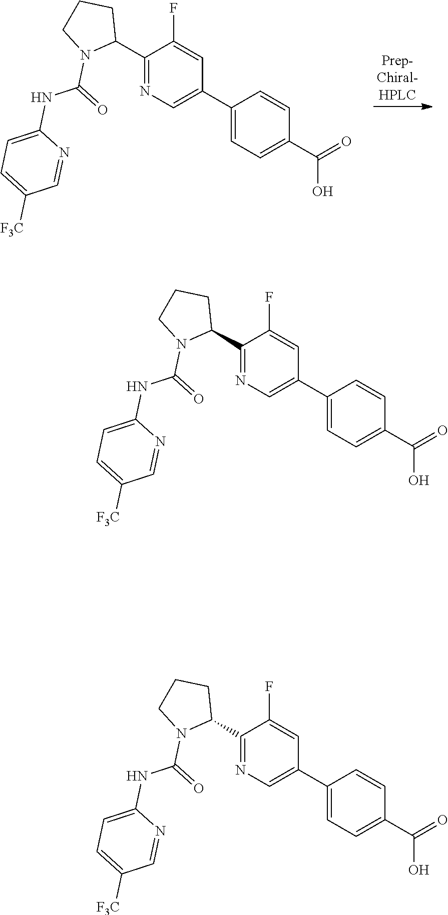 [SMILES: O=C(O)c1ccc(-c2cnc(C3CCCN3C(=O)Nc3ccc(C(F)(F)F)cn3)c(F)c2)cc1 ; O=C(O)c1ccc(-c2cnc([C@@H]3CCCN3C(=O)Nc3ccc(C(F)(F)F)cn3)c(F)c2)cc1 ; O=C(O)c1ccc(-c2cnc([C@H]3CCCN3C(=O)Nc3ccc(C(F)(F)F)cn3)c(F)c2)cc1]

4-(5-Fluoro-6-(1-((5-(trifluoromethyl)pyridin-2-yl)carbamoyl)pyrrolidin-2-yl)pyridin-3-yl)benzoic acid (40 mg) was resolved by preparative chiral HPLC (conditions: column: CHIRAL ART Cellulose-SC, 3*25 cm, 5 μm; mobile phase A: hexane (0.1% formic acid); mobile phase B: ethanol; flow rate: 40 mL/min; gradient (B %): isocratic 30% B; detector: 220/254 nm; RT1 (min): 7.2; RT2 (min): 8.3) to afford (Compound 90a; Method AA, Peak 1, 0.81 min), first peak, (S)-4-(5-fluoro-6-(1-((5-(trifluoromethyl)pyridin-2-yl)carbamoyl)pyrrolidin-2-yl)pyridin-3-yl)benzoic acid (14.0 mg, 35%) as a white solid and (Compound 90b; Method AA, Peak 2, 1.10 min), second peak, (R)-4-(5-fluoro-6-(1-((5-(trifluoromethyl)pyridin-2-yl)carbamoyl)pyrrolidin-2-yl)pyridin-3-yl)benzoic acid (13.9 mg, 35%) as a white solid.

| Cmpd # | Characterization Data |
|---|---|
| 90a | LCMS (ESI) [M + H]⁺: 475.1 |
| | ¹H NMR (400 MHz, DMSO-d₆) δ 9.39 (bs, 1H), 8.74 (s, 1H), 8.58 (bs, 1H), 8.10-7.96 (m, 5H), |
| | 7.88 (d, J = 7.2 Hz, 2H), 5.39 (bs, 1H), 3.86-3.81 (m, 1H), 3.76-3.71 (m, 1H), 2.37-2.32 (m, |
| | 1H), 2.16-1.93 (m, 3H). |
| 90b | LCMS (ESI) [M + H]⁺: 475.0 |
| | ¹H NMR (400 MHz, DMSO-d₆) δ 9.39 (bs, 1H), 8.74 (s, 1H), 8.57 (bs, 1H), 8.10-8.07 (m, 1H), |
| | 8.02 (d, J = 8.4 Hz, 2H), 7.98-7.95 (m, 2H), 7.88 (d, J = 8.4 Hz, 2H), 5.39 (bs, 1H), 3.87-3.82 |
| | (m, 1H), 3.77-3.72 (m, 1H), 2.42-2.33 (m, 1H), 2.18-1.93 (m, 3H). |

### Example 91: (S)-4-(5-fluoro-6-(1-((4-(trifluoromethyl)benzyl)carbamoyl)pyrrolidin-2-yl)pyridin-3-yl)benzoic acid and (R)-4-(5-fluoro-6-(1-((4-(trifluoromethyl)benzyl)carbamoyl)pyrrolidin-2-yl)pyridin-3-yl)benzoic acid

### Step 1: 4-(5-fluoro-6-(1-((4-(trifluoromethyl)benzyl)carbamoyl)pyrrolidin-2-yl)pyridin-3-yl)benzoic acid

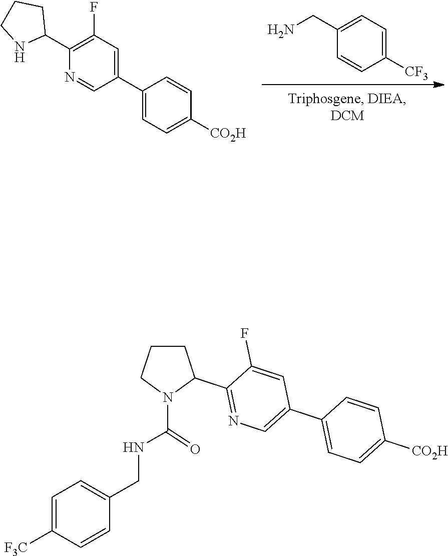 [SMILES: O=C(O)c1ccc(-c2cnc(C3CCCN3)c(F)c2)cc1 ; NCc1ccc(C(F)(F)F)cc1 ; O=C(O)c1ccc(-c2cnc(C3CCCN3C(=O)NCc3ccc(C(F)(F)F)cc3)c(F)c2)cc1]

To a solution of (4-(trifluoromethyl)phenyl)methanamine (61 mg, 0.349 mmol) and DIEA (226 mg, 1.75 mmol) in DCM (3.0 mL) at 0° C. was added triphosgene (42 mg, 0.143 mmol) dropwise. After 30 minutes, the mixture was then warmed to room temperature and 4-(5-fluoro-6-(pyrrolidin-2-yl)pyridin-3-yl)benzoic acid (100 mg, 0.350 mmol) was added dropwise over 3 minutes. After stirring for an additional 2 hours, the reaction mixture was cooled to 0° C. and quenched with ice water. The mixture was extracted with ethyl acetate (3×15 mL). The combined organic layers were dried over anhydrous Na₂SO₄, filtered and concentrated under reduced pressure. The crude residue was purified by reversed-phase flash chromatography (conditions: column: C18 silica gel; mobile phase A: water (0.1% formic acid), mobile phase B: acetonitrile; gradient (B %): 0% to 100% over 25 min; detector: UV 254 nm) to afford 4-(5-fluoro-6-(1-((4-(trifluoromethyl)benzyl)carbamoyl)pyrrolidin-2-yl)pyridin-3-yl)benzoic acid (100 mg, 59%) as a white solid. LCMS [M+H]⁺: 488.2

### Step 2: (S)-4-(5-fluoro-6-(1-((4-(trifluoromethyl)benzyl)carbamoyl)pyrrolidin-2-yl)pyridin-3-yl)benzoic acid and (R)-4-(5-fluoro-6-(1-((4-(trifluoromethyl)benzyl)carbamoyl)pyrrolidin-2-yl)pyridin-3-yl)benzoic acid

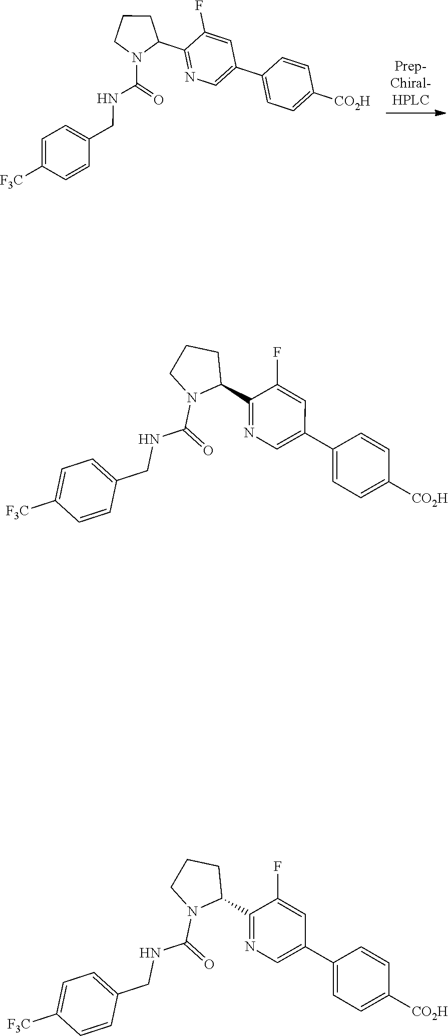 [SMILES: O=C(O)c1ccc(-c2cnc(C3CCCN3C(=O)NCc3ccc(C(F)(F)F)cc3)c(F)c2)cc1 ; O=C(O)c1ccc(-c2cnc([C@@H]3CCCN3C(=O)NCc3ccc(C(F)(F)F)cc3)c(F)c2)cc1 ; O=C(O)c1ccc(-c2cnc([C@H]3CCCN3C(=O)NCc3ccc(C(F)(F)F)cc3)c(F)c2)cc1]

4-(5-Fluoro-6-(1-((4-(trifluoromethyl)benzyl)carbamoyl)pyrrolidin-2-yl)pyridin-3-yl)benzoic acid (100 mg) was resolved by preparative chiral HPLC (conditions: column: CHIRAL ART Cellulose-SB, 3*25 cm, 5 μm; mobile phase A: hexane (0.1% formic acid), mobile phase B: ethanol; flow rate: 40 mL/min; gradient (B %): isocratic 30% B; detector: 220/254 nm; RT1 (min): 7.0; RT2 (min): 11.0) to afford (Compound 91a; Method M, Peak 1, 0.72 min), first peak, (S)-4-(5-fluoro-6-(1-((4-(trifluoromethyl)benzyl)carbamoyl)pyrrolidin-2-yl)pyridin-3-yl)benzoic acid (50.1 mg, 50%) as a white solid and (Compound 91b; Method M, Peak 2, 1.11 min), second peak, (R)-4-(5-fluoro-6-(1-((4-(trifluoromethyl)benzyl)carbamoyl)pyrrolidin-2-yl)pyridin-3-yl)benzoic acid (47.0 mg, 47%) as a white solid.

| Cmpd # | Characterization Data |
|---|---|
| 91a | LCMS (ESI) [M + H]⁺: 488.0 |
| | ¹H NMR (400 MHz, DMSO-d₆) δ 13.05 (bs, 1H), 8.76 (s, 1H), 8.06-8.03 (m, 3H), 7.89 (d, J = 8.4 |
| | Hz, 2H), 7.63 (d, J = 8.0 Hz, 2H), 7.40 (d, J = 8.0 Hz, 2H), 6.89 (t, J = 5.4 Hz, 1H), 5.25-5.22 (m, |
| | 1H), 4.27-4.25 (m, 1H), 4.22-4.20 (m, 1H), 3.65-3.60 (m, 1H), 3.52-3.48 (m, 1H), 2.34-2.29 |
| | (m, 1H), 2.06-1.96 (m, 2H), 1.88-1.84 (m, 1H). |
| 91b | LCMS (ESI) [M + H]⁺: 488.1 |
| | ¹H NMR (400 MHz, DMSO-d₆) δ 13.05 (bs, 1H), 8.76 (s, 1H), 8.06-8.03 (m, 3H), 7.89 (d, J = 8.4 |
| | Hz, 2H), 7.63 (d, J = 8.0 Hz, 2H), 7.41 (d, J = 8.0 Hz, 2H), 6.89 (t, J = 5.4 Hz, 1H), 5.25-5.22 (m, |
| | 1H), 4.31-4.26 (m, 1H), 4.22-4.18 (m, 1H), 3.64-3.61 (m, 1H), 3.60-3.48 (m, 1H), 2.34-2.29 |
| | (m, 1H), 2.11-1.95 (m, 2H), 1.88-1.84 (m, 1H). |

### Example 92: (S)-5-(4-(2-((4-isopropylphenyl)carbamoyl)-2-methylazetidine-1-carboxamido)phenyl)-6-methylpicolinic acid

### Step 1: tert-butyl (S)-2-((4-isopropylphenyl)carbamoyl)-2-methylazetidine-1-carboxylate

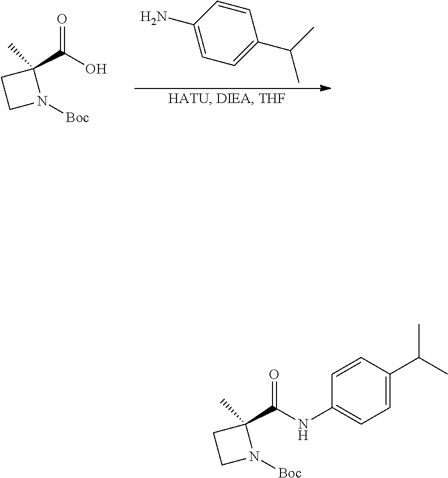

To (S)-1-(tert-butoxycarbonyl)-2-methylazetidine-2-carboxylic acid (100 mg, 0.465 mmol) in THF (5.0 mL) at 0° C. was added HATU (212 mg, 0.558 mmol) followed by the dropwise addition of DIEA (66 mg, 0.512 mmol). After 30 minutes, 4-isopropylaniline (63 mg, 0.467 mmol) was added dropwise. The mixture was warmed to room temperature and stirred for 2 hours. Upon completion, the reaction mixture was concentrated under reduced pressure. The crude residue was directly purified by silica gel column chromatography, eluted with petroleum ether/ethyl acetate (1:1), to afford tert-butyl (S)-2-((4-isopropylphenyl)carbamoyl)-2-methylazetidine-1-carboxylate (105 mg, 68%) as a white solid. LCMS [M+H]⁺: 333.2

### Step 2: (S)—N-(4-isopropylphenyl)-2-methylazetidine-2-carboxamide

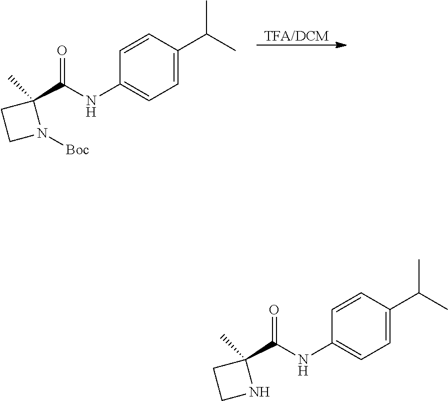 [SMILES: CC(C)c1ccc(NC(=O)[C@]2(C)CCN2C(=O)OC(C)(C)C)cc1 ; "TFA/DCM" ; CC(C)c1ccc(NC(=O)[C@]2(C)CCN2)cc1]

To a mixture of tert-butyl (S)-2-((4-isopropylphenyl)carbamoyl)-2-methylazetidine-1-carboxylate (95 mg, 0.286 mmol) in DCM (6.0 mL) at 0° C. was added TFA (3.0 mL) dropwise. The reaction mixture was warmed to room temperature and stirred for 1 hour. Upon completion, the resultant mixture was concentrated under reduced pressure to afford (S)—N-(4-isopropylphenyl)-2-methylazetidine-2-carboxamide (65 mg, crude) as a yellow solid, which was used directly in the next step without further purification. LCMS [M+H]⁺: 233.2

### Step 3: Methyl (S)-5-(4-(2-((4-isopropylphenyl)carbamoyl)-2-methylazetidine-1-carboxamido)phenyl)-6-methylpicolinate

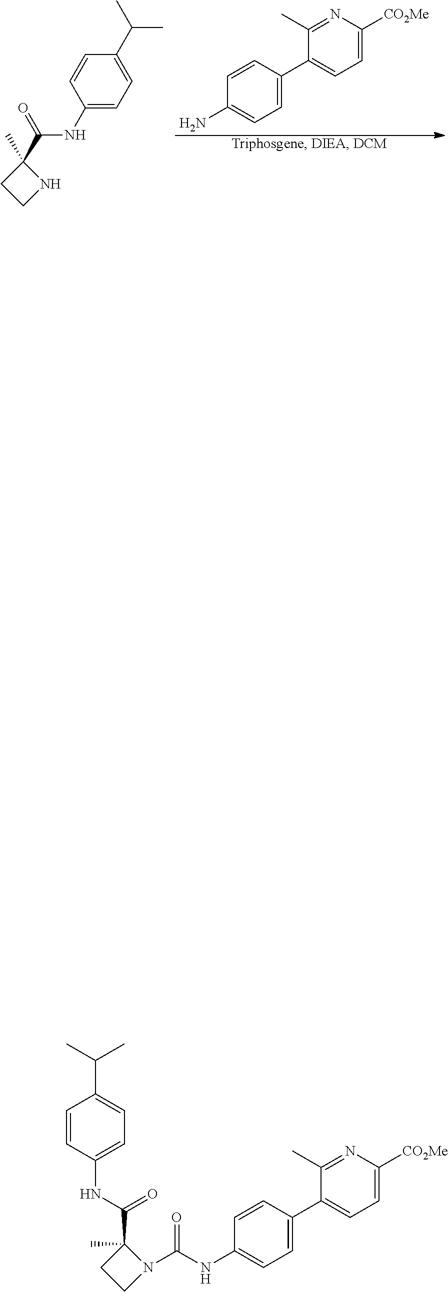 [SMILES: CC(C)c1ccc(NC(=O)[C@]2(C)CCN2)cc1 ; COC(=O)c1ccc(-c2ccc(N)cc2)c(C)n1 ; COC(=O)c1ccc(-c2ccc(NC(=O)N3CC[C@@]3(C)C(=O)Nc3ccc(C(C)C)cc3)cc2)c(C)n1]

To a solution of methyl 5-(4-aminophenyl)-6-methylpyridine-2-carboxylate (63 mg, 0.260 mmol) and DIEA (167 mg, 1.29 mmol) in DCM (5.0 mL) at 0° C. was added triphosgene (31 mg, 0.105 mmol) dropwise. After 30 minutes, the mixture was warmed to room temperature and (S)—N-(4-isopropylphenyl)-2-methylazetidine-2-carboxamide (60 mg, 0.259 mmol) was added dropwise over 2 minutes. After stirring for an additional 2 hours, the reaction was cooled to 0° C. and quenched with ice water. The resultant mixture was extracted with ethyl acetate (3×15 mL). The combined organic layers were dried over anhydrous Na₂SO₄, filtered and concentrated under reduced pressure. The crude residue was purified by silica gel column chromatography, eluted with petroleum ether/ethyl acetate (1:1), to afford methyl (S)-5-(4-(2-((4-isopropylphenyl)carbamoyl)-2-methylazetidine-1-carboxamido)phenyl)-6-methylpicolinate (62 mg, 48%) as a yellow solid. LCMS [M+H]⁺: 501.0

### Step 4: (S)-5-(4-(2-((4-isopropylphenyl)carbamoyl)-2-methylazetidine-1-carboxamido)phenyl)-6-methylpicolinic acid

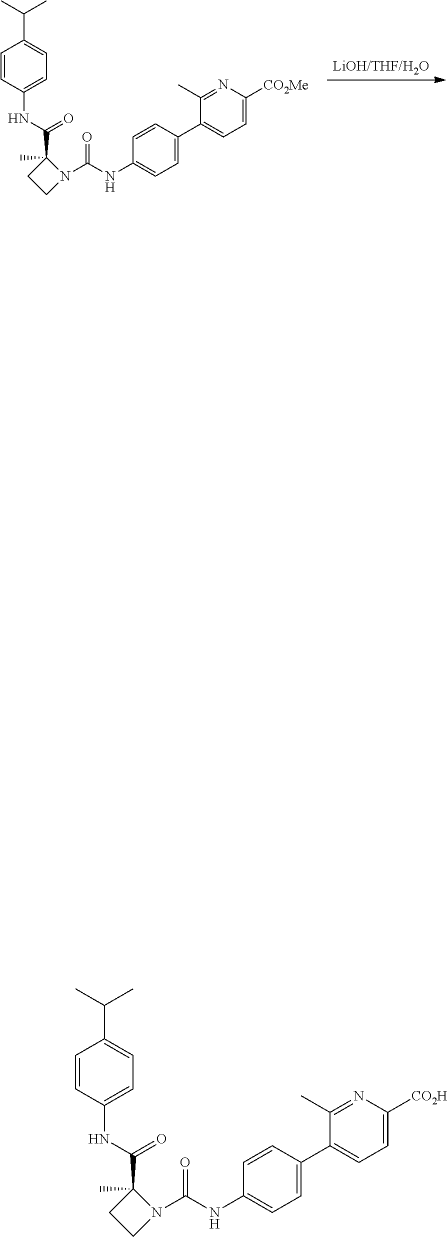 [SMILES: COC(=O)c1ccc(-c2ccc(NC(=O)N3CC[C@@]3(C)C(=O)Nc3ccc(C(C)C)cc3)cc2)c(C)n1 ; "LiOH/THF/H2O" ; Cc1nc(C(=O)O)ccc1-c1ccc(NC(=O)N2CC[C@@]2(C)C(=O)Nc2ccc(C(C)C)cc2)cc1]

To a solution of methyl (S)-5-(4-(2-((4-isopropylphenyl)carbamoyl)-2-methylazetidine-1-carboxamido)phenyl)-6-methylpicolinate (60.0 mg, 0.120 mmol) in THF (1.0 mL) and water (1.0 mL) at 0° C. was added LiOH monohydrate (12.6 mg, 0.300 mmol) portion-wise at room temperature. After 2 hours, the reaction mixture was concentrated under reduced pressure. The residue was directly purified by reversed-phase flash chromatography (conditions: column: C18 silica gel; mobile phase A: water (0.1% formic acid); mobile phase B: acetonitrile; gradient (B %): 0% to 100% over 25 min; detector: UV 200/220 nm) to afford (S)-5-(4-(2-((4-isopropylphenyl)carbamoyl)-2-methylazetidine-1-carboxamido)phenyl)-6-methylpicolinic acid (39.9 mg, 68%; Method R, Peak 1, 0.82 min—analyzed against Compound 93) as a white solid.

| Cmpd # | Characterization Data |
|---|---|
| 92 | LCMS (ESI) [M + H]⁺: 487.3 |
| | ¹H NMR (400 MHz, DMSO-d₆) δ 10.37 (s, 1H), 8.87 (bs, 1H), 7.93 (d, J = 7.6 Hz, 1H), 7.76 (d, J = |
| | 8.0 Hz, 1H), 7.67 (d, J = 8.4 Hz, 2H), 7.50 (d, J = 8.4 Hz, 2H), 7.38 (d, J = 8.4 Hz, 2H), 7.21 (d, J = |
| | 8.4 Hz, 2H), 4.11-4.09 (m, 1H), 4.07-4.05 (m, 1H), 2.89-2.82 (m, 1H), 2.67-2.62 (m, 1H), |
| | 2.52 (s, 3H), 2.11-2.06 (m, 1H), 1.73 (s, 3H), 1.18 (d, J = 7.2 Hz, 6H). |

### Example 93: (R)-5-(4-(2-((4-isopropylphenyl)carbamoyl)-2-methylazetidine-1-carboxamido)phenyl)-6-methylpicolinic acid

### Step 1: tert-butyl (R)-2-((4-isopropylphenyl)carbamoyl)-2-methylazetidine-1-carboxylate

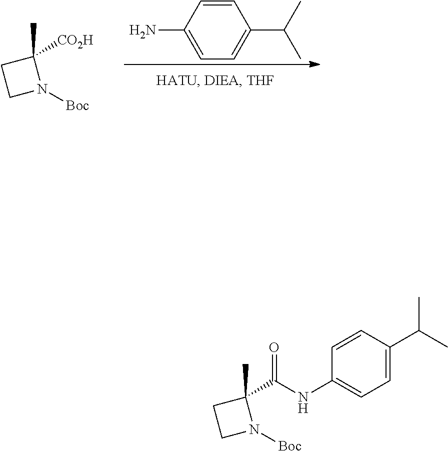

To (R)-1-(tert-butoxycarbonyl)-2-methylazetidine-2-carboxylic acid (1.59 g, 7.4 mmol) in THF (15.0 mL) at 0° C. was added HATU (3.09 g, 8.1 mmol) followed by the dropwise addition of DIEA (1.15 g, 8.9 mmol). After 30 minutes, 4-isopropylaniline (1.00 g, 7.4 mmol) was added dropwise. The reaction was warmed to room temperature and stirred for an additional 2 hours. Upon completion, the mixture was concentrated under reduced pressure. The crude residue was directly purified by silica gel column chromatography, eluted with petroleum ether/ethyl acetate (1:1), to afford tert-butyl (R)-2-((4-isopropylphenyl)carbamoyl)-2-methylazetidine-1-carboxylate (1.20 g, 49%) as a brown solid. LCMS [M+H]⁺: 333.2

### Step 2: (R)—N-(4-isopropylphenyl)-2-methylazetidine-2-carboxamide

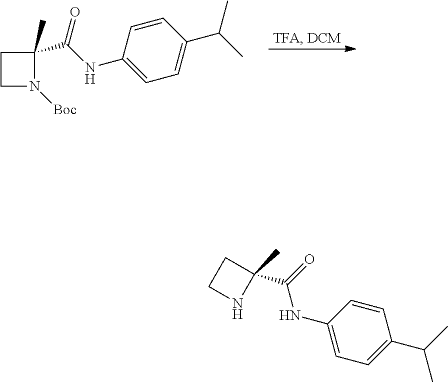 [SMILES: CC(C)c1ccc(NC(=O)[C@@]2(C)CCN2C(=O)OC(C)(C)C)cc1 ; CC(C)c1ccc(NC(=O)[C@@]2(C)CCN2)cc1]

To a mixture of tert-butyl (R)-2-((4-isopropylphenyl)carbamoyl)-2-methylazetidine-1-carboxylate (110 mg, 0.331 mmol) in DCM (6.0 mL) at 0° C. was added TFA (3.0 mL) dropwise. The resultant mixture was warmed to room temperature and stirred for 1 hour. Upon completion, the mixture was concentrated under reduced pressure to afford (R)—N-(4-isopropylphenyl)-2-methylazetidine-2-carboxamide (90 mg, crude) as a yellow oil, which was used directly in the next step without further purification. LCMS [M+H]⁺: 233.2

### Step 3: methyl (R)-5-(4-(2-((4-isopropylphenyl)carbamoyl)-2-methylazetidine-1-carboxamido)phenyl)-6-methylpicolinate

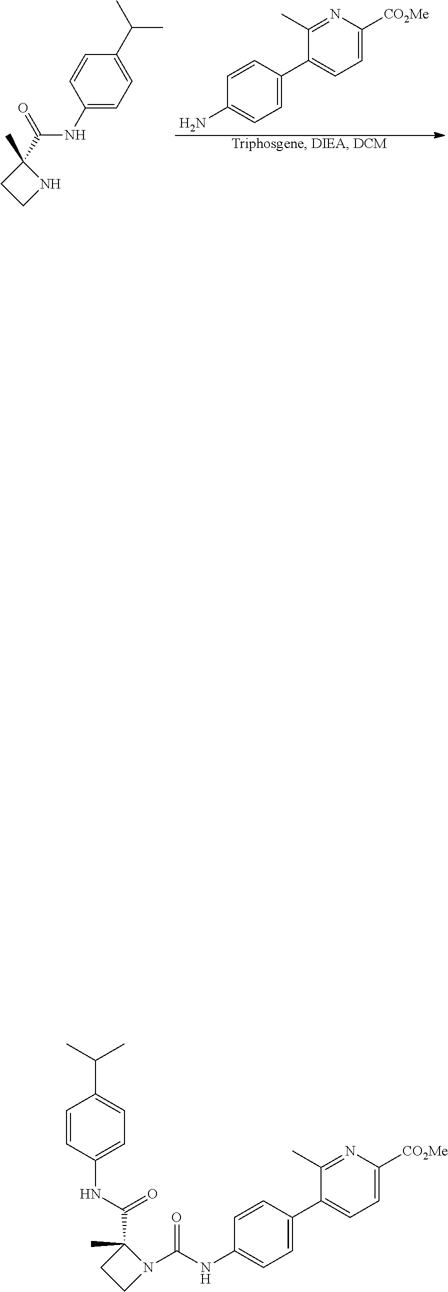 [SMILES: CC(C)c1ccc(NC(=O)[C@@]2(C)CCN2)cc1 ; COC(=O)c1ccc(-c2ccc(N)cc2)c(C)n1 ; COC(=O)c1ccc(-c2ccc(NC(=O)N3CC[C@]3(C)C(=O)Nc3ccc(C(C)C)cc3)cc2)c(C)n1]

To a solution of methyl 5-(4-aminophenyl)-6-methylpicolinate (83 mg, 0.343 mmol) and DIEA (134 mg, 1.04 mmol) in DCM (5.0 mL) at 0° C. was added triphosgene (33.5 mg, 0.114 mmol) dropwise. After 30 minutes, the reaction was warmed to room temperature and (R)—N-(4-isopropylphenyl)-2-methylazetidine-2-carboxamide (80 mg, 0.345 mmol) was added dropwise over 2 min. The reaction was allowed to stir for an additional 2 hours. Upon completion, the mixture was cooled to 0° C. and quenched with ice water. The mixture was extracted with ethyl acetate (3×15 mL). The combined organic layers were dried over anhydrous Na₂SO₄, filtered and concentrated under reduces pressure. The crude residue was purified by silica gel column chromatography, eluted with DCM/MeOH (5:1), to afford methyl (R)-5-(4-(2-((4-isopropylphenyl)carbamoyl)-2-methylazetidine-1-carboxamido)phenyl)-6-methylpicolinate (110 mg, 64%) as a yellow solid. LCMS [M+H]⁺: 501.2

### Step 4: (R)-5-(4-(2-((4-isopropylphenyl)carbamoyl)-2-methylazetidine-1-carboxamido)phenyl)-6-methylpicolinic acid

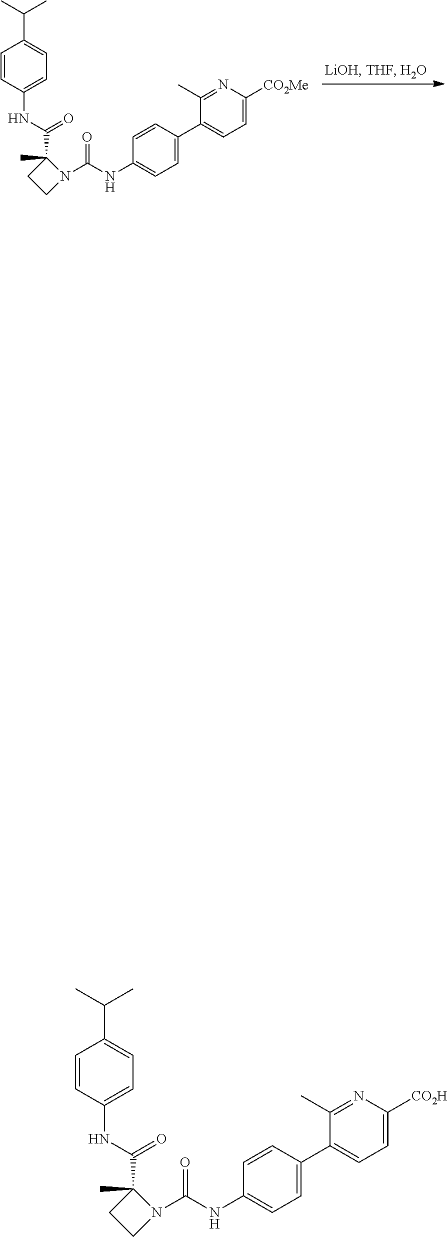

To a mixture of methyl (R)-5-(4-(2-((4-isopropylphenyl)carbamoyl)-2-methylazetidine-1-carboxamido)phenyl)-6-methylpicolinate (100 mg, 0.200 mmol) in THF (3.0 mL) and water (3.0 mL) at 0° C. was added LiOH monohydrate (25 mg, 0.595 mmol) portion-wise. The resultant mixture was warmed to room temperature and stirred for 2 hours. Upon completion, the mixture concentrated under reduced pressure. The crude residue was purified by reversed-phase flash chromatography (conditions: column: C18 silica gel; mobile phase A, water (0.1% formic acid), mobile phase B: acetonitrile; gradient (B %): 0% to 100% over 25 min; detector: UV 200/220 nm) to afford (R)-5-(4-(2-((4-isopropylphenyl)carbamoyl)-2-methylazetidine-1-carboxamido)phenyl)-6-methylpicolinic acid (43.3 mg, 45%; Method R, Peak 2, 2.10 min—analyzed against Compound 92) as a white solid.

| Cmpd # | Characterization Data |
|---|---|
| 93 | LCMS (ESI) [M + H]⁺: 487.3 |
| | ¹H NMR (400 MHz, DMSO-d₆) δ 10.37 (s, 1H), 8.87 (bs, 1H), 7.93 (d, J = 7.6 Hz, 1H), 7.77 (d, J = |
| | 8.0 Hz, 1H), 7.67 (d, J = 8.4 Hz, 2H), 7.50 (d, J = 8.4 Hz, 2H), 7.37 (d, J = 8.4 Hz, 2H), 7.20 |
| | (d, J = 8.4 Hz, 2H), 4.11-4.05 (m, 1H), 3.93-3.87 (m, 1H), 2.89-2.82 (m, 1H), 2.67-2.60 (m, |
| | 1H), 2.52 (s, 3H), 2.12-2.09 (m, 1H), 1.75 (s, 3H), 1.18 (d, J = 7.2 Hz, 6H). |

### Example 94: (S)-5-(4-(2-((4-isopropylphenyl)carbamoyl)-3,3-dimethylazetidine-1-carboxamido)phenyl)-6-methylpicolinic acid

### Step 1: (S)-1-((4-(6-(methoxycarbonyl)-2-methylpyridin-3-yl)phenyl)carbamoyl)-3,3-dimethylazetidine-2-carboxylic acid

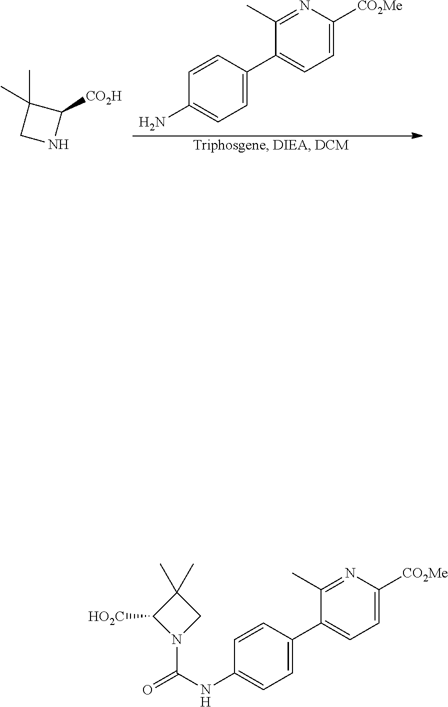

To a mixture of methyl 5-(4-aminophenyl)-6-methylpicolinate (94 mg, 0.388 mmol) and DIEA (100 mg, 0.775 mmol) in DCM (3.0 mL) at 0° C. was added triphosgene (46 mg, 0.156 mmol) dropwise. After 30 minutes, (S)-3,3-dimethylazetidine-2-carboxylic acid (50 mg, 0.388 mmol) was added dropwise over 2 minutes. The resultant mixture was warmed to room temperature stirred for 2 hours. Upon completion the reaction mixture was cooled to 0° C. and quenched with ice water. The mixture was extracted with ethyl acetate (3×15 mL). The combined organic layers were dried over anhydrous Na₂SO₄, filtered and concentrated under reduced pressure. The crude residue was purified by silica gel column chromatography, eluted with DCM/MeOH (5:1), to afford (S)-1-((4-(6-(methoxycarbonyl)-2-methylpyridin-3-yl)phenyl)carbamoyl)-3,3-dimethylazetidine-2-carboxylic acid (78 mg, 51%) as a yellow solid. LCMS [M+H]⁺: 398.2

### Step 2: methyl (S)-5-(4-(2-((4-isopropylphenyl)carbamoyl)-3,3-dimethylazetidine-1-carboxamido)phenyl)-6-methylpicolinate

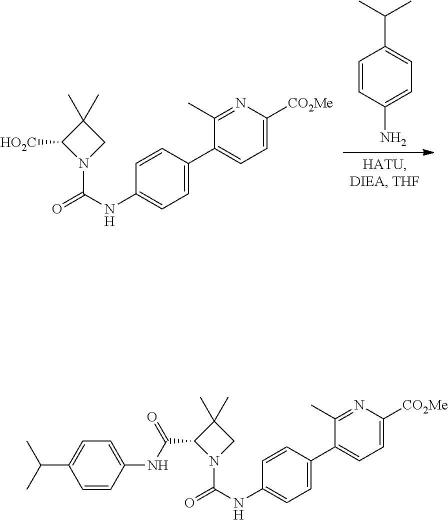

To a mixture of (S)-1-((4-(6-(methoxycarbonyl)-2-methylpyridin-3-yl)phenyl)carbamoyl)-3,3-dimethylazetidine-2-carboxylic acid (68 mg, 0.171 mmol) in THF (3.0 mL) at 0° C. was added HATU (72 mg, 0.189 mmol) followed by the dropwise addition of DIEA (27 mg, 0.209 mmol). After 30 minutes, 4-isopropylaniline (23 mg, 0.170 mmol) was added dropwise. The resultant mixture was warmed to room temperature for stirred for an additional 1 hour. Upon completion, the mixture was concentrated under reduced pressure. The crude residue was purified by silica gel column chromatography, eluted with petroleum ether/ethyl acetate (1:10), to afford methyl (S)-5-(4-(2-((4-isopropylphenyl)carbamoyl)-3,3-dimethylazetidine-1-carboxamido)phenyl)-6-methylpicolinate (61 mg, 69%) as a white solid. LCMS [M+H]⁺: 515.3

### Step 3: (S)-5-(4-(2-((4-isopropylphenyl)carbamoyl)-3,3-dimethylazetidine-1-carboxamido)phenyl)-6-methylpicolinic acid

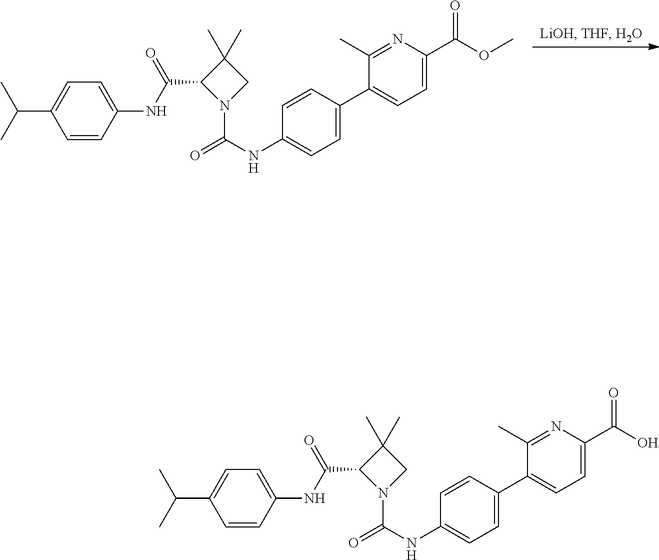

To a mixture of methyl (S)-5-(4-(2-((4-isopropylphenyl)carbamoyl)-3,3-dimethylazetidine-1-carboxamido)phenyl)-6-methylpicolinate (50 mg, 0.0973 mmol) in THF (1.0 mL) and water (1.0 mL) at 0° C. was added LiOH monohydrate (12.6 mg, 0.300 mmol) portion-wise. The resultant mixture was warmed to room temperature and stirred for 2 hours. Upon completion, the mixture was concentrated under reduced pressure. The crude residue was directly purified by preparative HPLC (conditions: column: Xselect CSH Prep C18, 30*150 mm 5 μm; mobile phase A: water (0.1% formic acid), mobile phase B: acetonitrile; flow rate: 60 mL/min; gradient (B %): 40% B to 47% B over 9 min; detector: 254/220 nm; RT1 (min): 8.8) to afford (S)-5-(4-(2-((4-isopropylphenyl)carbamoyl)-3,3-dimethylazetidine-1-carboxamido)phenyl)-6-methylpicolinic acid (28.5 mg, 59%; Method R, Peak 1, 0.86 min—analyzed against Compound 95) as a white solid.

| Cmpd # | Characterization Data |
|---|---|
| 94 | LCMS (ESI) [M + H]⁺: 501.3 |
| | ¹H NMR (400 MHz, DMSO-d₆) δ 9.77 (s, 1H), 8.69 (s, 1H), 7.91 (d, J = 7.6 Hz, 1H), 7.75 (d, J = 7.6 |
| | Hz, 1H), 7.65 (d, J = 8.8 Hz, 2H), 7.55 (d, J = 8.4 Hz, 2H), 7.34 (d, J = 8.4 Hz, 2H), 7.18 (d, J = 8.4 |
| | Hz, 2H), 4.40 (s, 1H), 3.78 (d, J = 7.2 Hz, 1H), 3.68 (d, J = 7.2 Hz, 1H), 2.87-2.81 (m, 1H), 2.52- |
| | 2.51 (m, 3H), 1.40 (s, 3H), 1.18 (d, J = 6.8 Hz, 6H), 1.11 (s, 3H). |

### Example 95: (R)-5-(4-(2-((4-isopropylphenyl)carbamoyl)-3,3-dimethylazetidine-1-carboxamido)phenyl)-6-methylpicolinic acid

### Step 1: (R)-1-((4-(6-(methoxycarbonyl)-2-methylpyridin-3-yl)phenyl)carbamoyl)-3,3-dimethylazetidine-2-carboxylic acid

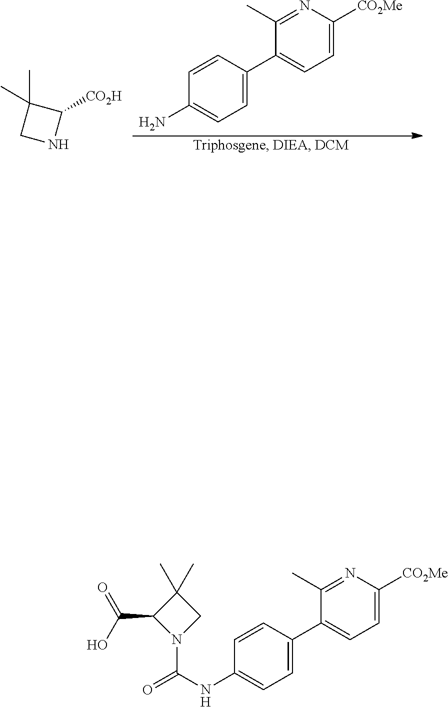

To a mixture of methyl 5-(4-aminophenyl)-6-methylpicolinate (113 mg, 0.467 mmol) and DIEA (175 mg, 1.36 mmol) in DCM (5.0 mL) at 0° C. was added triphosgene (46 mg, 0.156 mmol) dropwise. After 30 minutes, (R)-3,3-dimethylazetidine-2-carboxylic acid (50 mg, 0.388 mmol) was added dropwise over 2 minutes. The reaction mixture was warmed to room temperature and stirred for an additional 2 hours. Upon completion, the reaction mixture was cooled to 0° C., quenched with ice water, and extracted with ethyl acetate (3×7 mL). The combined organic layers were dried over anhydrous Na₂SO₄, filtered and concentrated under reduced pressure. The crude residue was purified by silica gel column chromatography, eluted with DCM/MeOH (5:1), to afford (R)-1-((4-(6-(methoxycarbonyl)-2-methylpyridin-3-yl)phenyl)carbamoyl)-3,3-dimethylazetidine-2-carboxylic acid (98 mg, 64%) as a yellow solid. LCMS [M+H]⁺: 398.2

### Step 2: methyl (R)-5-(4-(2-((4-isopropylphenyl)carbamoyl)-3,3-dimethylazetidine-1-carboxamido)phenyl)-6-methylpicolinate

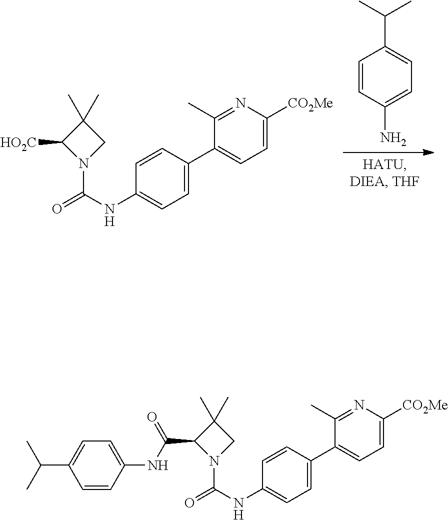

To a mixture of (R)-1-((4-(6-(methoxycarbonyl)-2-methylpyridin-3-yl)phenyl)carbamoyl)-3,3-dimethylazetidine-2-carboxylic acid (88 mg, 0.222 mmol) in THF (6.0 mL) at 0° C. was added HATU (126 mg, 0.332 mmol) followed by the dropwise addition of DIEA (86 mg, 0.667 mmol). After 30 minutes, 4-isopropylaniline (36 mg, 0.267 mmol) was added dropwise. The reaction was warmed to room temperature and stirred for an additional 1 hour. Upon completion, the reaction mixture was concentrated under reduced pressure. The crude residue was purified by silica gel column chromatography, eluted with petroleum ether/ethyl acetate (1:1) to afford methyl (R)-5-(4-(2-((4-isopropylphenyl)carbamoyl)-3,3-dimethylazetidine-1-carboxamido)phenyl)-6-methylpicolinate (110 mg, 97%) as a white solid. LCMS [M+H]⁺: 515.3

### Step 3: (R)-5-(4-(2-((4-isopropylphenyl)carbamoyl)-3,3-dimethylazetidine-1-carboxamido)phenyl)-6-methylpicolinic acid

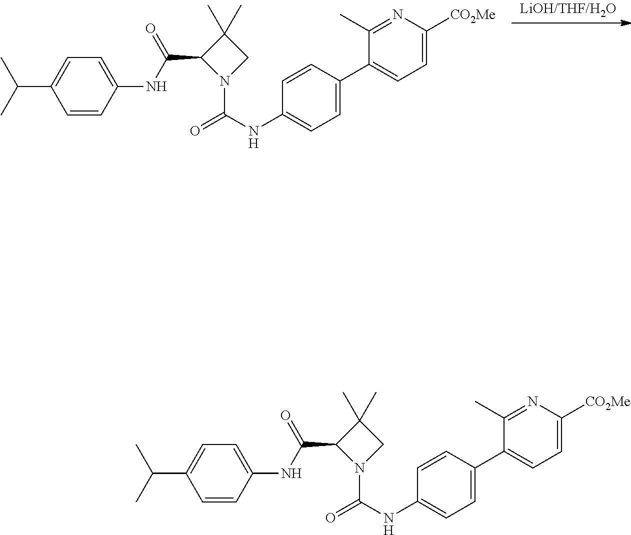 [SMILES: COC(=O)c1ccc(-c2ccc(NC(=O)N3CC(C)(C)[C@@H]3C(=O)Nc3ccc(C(C)C)cc3)cc2)c(C)n1 ; COC(=O)c1ccc(-c2ccc(NC(=O)N3CC(C)(C)[C@@H]3C(=O)Nc3ccc(C(C)C)cc3)cc2)c(C)n1]

To a mixture of methyl (R)-5-(4-(2-((4-isopropylphenyl)carbamoyl)-3,3-dimethylazetidine-1-carboxamido)phenyl)-6-methylpicolinate (100 mg, 0.195 mmol) in THF (3.0 mL) and water (3.0 mL) at 0° C. was added LiOH monohydrate (25.2 mg, 0.600 mmol) portion-wise. The reaction was warmed to room temperature and stirred for 2 hours. Upon completion, the mixture was concentrated under reduced pressure. The crude residue was directly purified by reversed-phase flash chromatography (conditions: column: C18 silica gel; mobile phase A: water (0.1% formic acid), mobile phase B: acetonitrile; gradient (B %): 0% to 100% over 25 min; detector: UV 254 nm) to afford (R)-5-(4-(2-((4-isopropylphenyl)carbamoyl)-3,3-dimethylazetidine-1-carboxamido)phenyl)-6-methylpicolinic acid (13.4 mg, 14%; Method R, Peak 2, 1.57 min—analyzed against Compound 94) as a white solid.

| Cmpd # | Characterization Data |
|---|---|
| 95 | LCMS (ESI) [M + H]⁺: 501.2 |
| | ¹H NMR (400 MHz, DMSO-d₆) δ 9.76 (s, 1H), 8.69 (s, 1H), 7.91 (d, J = 8.0 Hz, 1H), 7.76 (d, J = |
| | 8.0 Hz, 1H), 7.65 (d, J = 8.8 Hz, 2H), 7.55 (d, J = 8.4 Hz, 2H), 7.34 (d, J = 8.4 Hz, 2H), 7.18 (d, J = |
| | 8.4 Hz, 2H), 4.40 (s, 1H), 3.78 (d, J = 7.2 Hz, 1H), 3.68 (d, J = 7.2 Hz, 1H), 2.88-2.81 (m, 1H), |
| | 2.57-2.51 (m, 3H), 1.40 (s, 3H), 1.18 (d, J = 6.8 Hz, 6H), 1.11 (s, 3H). |

### Example 96: (R)-4-(5-fluoro-6-(1-((4-isobutylphenyl)carbamoyl)pyrrolidin-2-yl)pyridin-3-yl)benzoic acid and (S)-4-(5-fluoro-6-(1-((4-isobutylphenyl)carbamoyl)pyrrolidin-2-yl)pyridin-3-yl)benzoic acid

### Step 1: 4-(5-fluoro-6-(1-((4-isobutylphenyl)carbamoyl)pyrrolidin-2-yl)pyridin-3-yl)benzoic acid

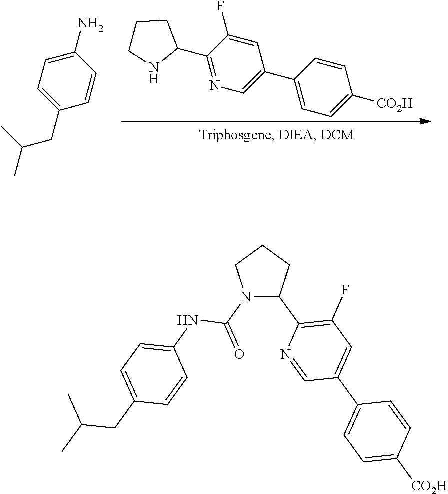

A solution of triphosgene (64 mg, 0.218 mmol) in DCM (4.0 mL) at 0° C. was treated with 4-isobutylaniline (80 mg, 0.537 mmol) and DIEA (346 mg, 2.68 mmol). After 15 minutes, 4-(5-fluoro-6-(pyrrolidin-2-yl)pyridin-3-yl)benzoic acid (153 mg, 0.535 mmol) was added. The resultant mixture was warmed to room temperature and stirred for 2 hours. Upon completion, the reaction was cooled to 0° C. and quenched by the addition of saturated ammonium chloride (10 mL). The resulting mixture was extracted with DCM (3×15 mL). The combined organic layers were washed with water, dried over anhydrous Na₂SO₄, filtered and concentrated under reduced pressure. The crude residue was purified by silica gel column chromatography, eluted with DCM/MeOH (5:1), to afford 4-(5-fluoro-6-(1-((4-isobutylphenyl)carbamoyl)pyrrolidin-2-yl)pyridin-3-yl)benzoic acid (120 mg, 49%) as a pale yellow solid. LCMS (ESI) [M+H]⁺: 403.2

### Step 2: (R)-4-(5-fluoro-6-(1-((4-isobutylphenyl)carbamoyl)pyrrolidin-2-yl)pyridin-3-yl)benzoic acid and (S)-4-(5-fluoro-6-(1-((4-isobutylphenyl)carbamoyl)pyrrolidin-2-yl)pyridin-3-yl)benzoic acid

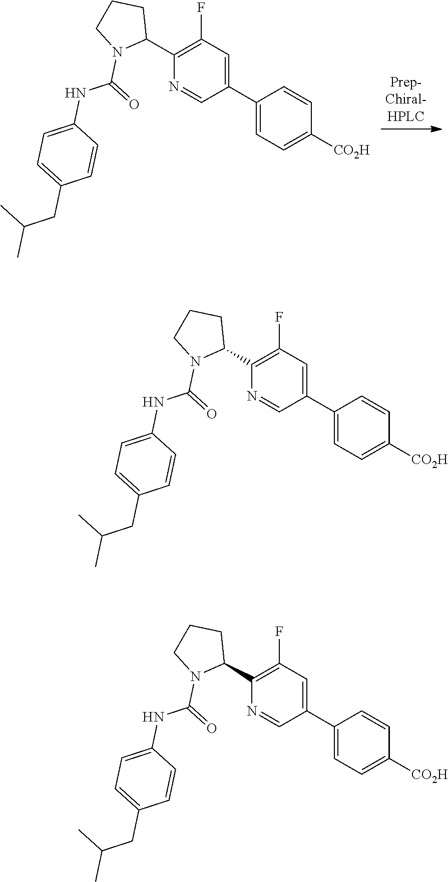 [SMILES: CC(C)Cc1ccc(NC(=O)N2CCCC2c2ncc(-c3ccc(C(=O)O)cc3)cc2F)cc1 ; CC(C)Cc1ccc(NC(=O)N2CCC[C@@H]2c2ncc(-c3ccc(C(=O)O)cc3)cc2F)cc1 ; CC(C)Cc1ccc(NC(=O)N2CCC[C@H]2c2ncc(-c3ccc(C(=O)O)cc3)cc2F)cc1]

4-(5-Fluoro-6-(1-((4-isobutylphenyl)carbamoyl)pyrrolidin-2-yl)pyridin-3-yl)benzoic acid (117 mg) was purified by preparative chiral-HPLC (conditions: column: CHIRAL ART Cellulose-SC, 3*25 cm, 5 μm; mobile phase A: hexane (0.1% formic acid); mobile phase B: ethanol; flow rate: 40 mL/min; gradient (B %): isocratic 30% B; detector: 220/254 nm; RT1 (min): 8.8; RT2 (min): 10.7) to afford first peak (Compound 96a; Method AA, Peak 2, 1.48 min) (R)-4-(5-fluoro-6-(1-((4-isobutylphenyl)carbamoyl)pyrrolidin-2-yl)pyridin-3-yl)benzoic acid (44.2 mg, 38%) as a white solid and second peak (Compound 96b; Method AA, Peak 1, 1.11 min) (S)-4-(5-fluoro-6-(1-((4-isobutylphenyl)carbamoyl)pyrrolidin-2-yl)pyridin-3-yl)benzoic acid (51.0 mg, 44%) as a white solid.

| Cmpd # | Characterization Data |
|---|---|
| 96a | LCMS (ESI) [M + H]⁺: 462.2 |
| | ¹H NMR (400 MHz, DMSO-d₆) δ 13.09 (bs, 1H) 8.73 (s, 1H), 8.14 (s, 1H), 8.10-7.99 (m, 3H), 7.88 |
| | (d, J = 8.1 Hz, 2H), 7.32 (d, J = 8.2 Hz, 2H), 6.95 (d, J = 8.2 Hz, 2H), 5.37-5.30 (m, 1H), 3.80- |
| | 3.71 (m, 1H), 3.68-3.57 (m, 1H), 2.41-2.28 (m, 3H), 2.20-1.96 (m, 2H), 1.94-1.85 (m, 1H), |
| | 1.82-1.67 (m, 1H), 0.82 (d, J = 6.6 Hz, 6H). |
| 96b | LCMS (ESI) [M + H]⁺: 462.2 |
| | ¹H NMR (400 MHz, DMSO-d₆) δ 13.11 (bs, 1H), 8.73 (s, 1H), 8.14 (s, 1H), 8.10-7.99 (m, 3H), |
| | 7.88 (d, J = 8.1 Hz, 2H), 7.32 (d, J = 8.2 Hz, 2H), 6.95 (d, J = 8.2 Hz, 2H), 5.37-5.30 (m, 1H), |
| | 3.80-3.71 (m, 1H), 3.67-3.57 (m, 1H), 2.40-2.28 (m, 3H), 2.19-2.07 (m, 1H), 2.06-1.95 (m, 1H), |
| | 1.94-1.84 (m, 1H), 1.81-1.67 (m, 1H), 0.82 (d, J = 6.6 Hz, 6H). |

### Example 97: (S)-4-(5-fluoro-6-(1-((4-isopropylphenyl)carbamoyl)-2-methylazetidin-2-yl)pyridin-3-yl)benzoic acid & (R)-4-(5-fluoro-6-(1-((4-isopropylphenyl)carbamoyl)-2-methylazetidin-2-yl)pyridin-3-yl)benzoic acid

### Step 1: tert-butyl 2-(3-fluoro-5-(4-(methoxycarbonyl)phenyl)pyridin-2-yl)-2-methylazetidine-1-carboxylate

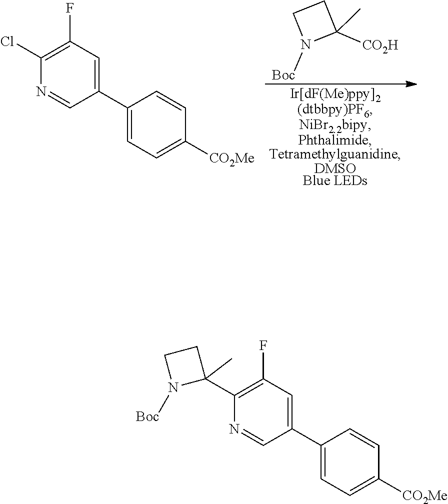

To a solution of 1-(tert-butoxycarbonyl)-2-methylazetidine-2-carboxylic acid (1.22 g, 5.65 mmol), methyl 4-(6-chloro-5-fluoropyridin-3-yl)benzoate (1.50 g, 5.65 mmol), and [4,4′-Bis(tert-butyl)-2,2′-bipyridine]nickel dibromide (0.270 g, 0.565 mmol) in DMSO (20.0 mL) at room temperature was added sequentially [Ir(dF(Me)ppy)₂(dtbbpy)]PF₆ (60.0 mg, 56.0 μmol), 2,3-dihydro-1H-isoindole-1,3-dione (1.25 g, 8.47 mmol), and tetramethylguanidine (0.650 g, 5.65 mmol). The resultant mixture was stirred for 24 hours under 450 nm blue LED irradiation. Upon completion, the mixture was diluted with water (50 mL) and extracted with ethyl acetate (6×20 mL). The combined organic layers were washed with water (5×4 mL), dried over anhydrous sodium sulfate, filtered, and concentrated under reduced pressure. The crude residue was purified by silica gel column chromatography, eluted with petroleum ether/ethyl acetate (5:1), to afford tert-butyl 2-(3-fluoro-5-(4-(methoxycarbonyl)phenyl)pyridin-2-yl)-2-methylazetidine-1-carboxylate (200 mg, 9%) as a yellow-green oil. LCMS (ESI) [M+H]⁺: 401.4

### Step 2: methyl 4-(5-fluoro-6-(2-methylazetidin-2-yl)pyridin-3-yl)benzoate

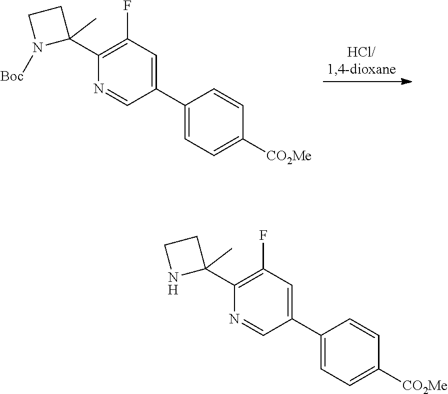 [SMILES: COC(=O)c1ccc(-c2cnc(C3(C)CCN3C(=O)OC(C)(C)C)c(F)c2)cc1 ; COC(=O)c1ccc(-c2cnc(C3(C)CCN3)c(F)c2)cc1]

To a solution of tert-butyl 2-(3-fluoro-5-(4-(methoxycarbonyl)phenyl)pyridin-2-yl)-2-methylazetidine-1-carboxylate (200 mg, 0.499 mmol) in dioxane (3.0 mL) at room temperature was added HCl (3.0 mL, 12 mmol, 4.0 M in 1,4-dioxane). The resultant solution was stirred for 1 hour. Upon completion, the mixture was concentrated under reduced pressure to afford methyl 4-[5-fluoro-6-(2-methylazetidin-2-yl)pyridin-3-yl]benzoate (150 mg, crude) as a yellow solid, which was used in the next step without further purification. LCMS (ESI) [M+H]⁺: 301.3

### Step 3: methyl 4-(5-fluoro-6-(1-((4-isopropylphenyl)carbamoyl)-2-methylazetidin-2-yl)pyridin-3-yl)benzoate

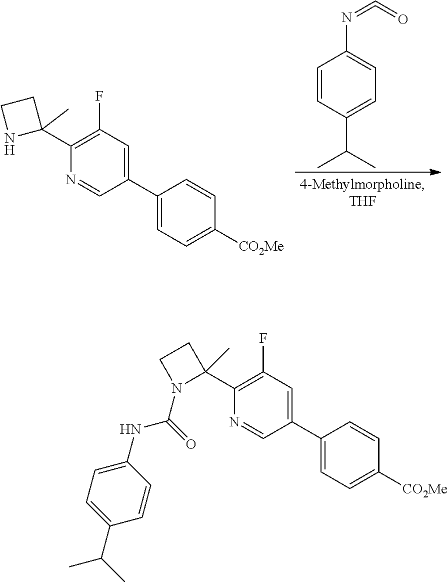 [SMILES: COC(=O)c1ccc(-c2cnc(C3(C)CCN3)c(F)c2)cc1 ; CC(C)c1ccc(N=C=O)cc1 ; COC(=O)c1ccc(-c2cnc(C3(C)CCN3C(=O)Nc3ccc(C(C)C)cc3)c(F)c2)cc1]

A solution of 1-isocyanato-4-isopropylbenzene (96.6 mg, 0.599 mmol), methyl 4-[5-fluoro-6-(2-methylazetidin-2-yl)pyridin-3-yl]benzoate (150 mg, 0.499 mmol) and 4-methylmorpholine (152 mg, 1.50 mmol) in THF (5.0 mL) was stirred at room temperature for 1 hour. Upon completion, the mixture was concentrated under reduced pressure. The crude residue was purified by silica gel column chromatography, eluted with ethyl acetate/petroleum ether (1:1) to afford methyl 4-(5-fluoro-6-(1-((4-isopropylphenyl)carbamoyl)-2-methylazetidin-2-yl)pyridin-3-yl)benzoate (100 mg, 43%) as a yellow solid. LCMS (ESI) [M+H]⁺: 462.2

### Step 4: 4-(5-fluoro-6-(1-((4-isopropylphenyl)carbamoyl)-2-methylazetidin-2-yl)pyridin-3-yl)benzoic acid

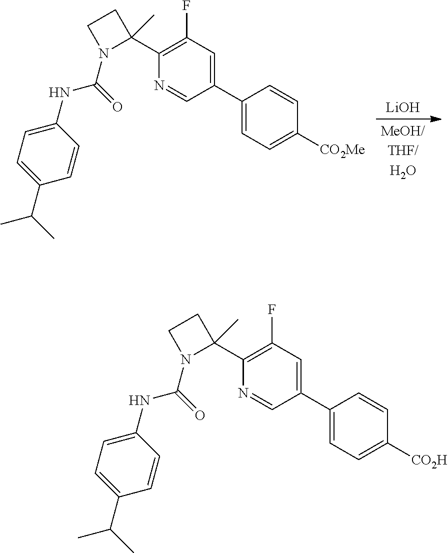 [SMILES: COC(=O)c1ccc(-c2cnc(C3(C)CCN3C(=O)Nc3ccc(C(C)C)cc3)c(F)c2)cc1 ; "LiOH" ; CC(C)c1ccc(NC(=O)N2CCC2(C)c2ncc(-c3ccc(C(=O)O)cc3)cc2F)cc1]

A solution of methyl 4-(5-fluoro-6-(1-((4-isopropylphenyl)carbamoyl)-2-methylazetidin-2-yl)pyridin-3-yl)benzoate (100 mg, 0.217 mmol) and lithium hydroxide monohydrate (45.4 mg, 1.08 mmol) in methanol (1.0 mL), THF (2.0 mL) and water (2.0 mL) was stirred at room temperature for 1 hour. Upon completion, the mixture was concentrated under reduced pressure and the crude residue was purified by reversed-phase flash chromatography (conditions: column: C18 silica gel; mobile phase: water (0.1% formic acid); mobile phase B: acetonitrile; gradient (B %): 10% to 80% over 20 min; detector: UV 254 nm) to afford 4-(5-fluoro-6-(1-((4-isopropylphenyl)carbamoyl)-2-methylazetidin-2-yl)pyridin-3-yl)benzoic acid (60 mg, 62%) as a white solid. LCMS (ESI) [M+H]⁺: 448.5

### Step 5: (S)-4-(5-fluoro-6-(1-((4-isopropylphenyl)carbamoyl)-2-methylazetidin-2-yl)pyridin-3-yl)benzoic acid & (R)-4-(5-fluoro-6-(1-((4-isopropylphenyl)carbamoyl)-2-methylazetidin-2-yl)pyridin-3-yl)benzoic acid

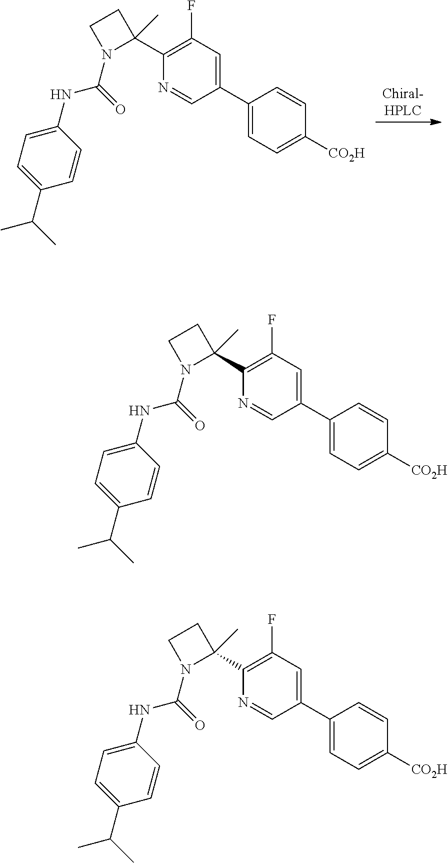 [SMILES: CC(C)c1ccc(NC(=O)N2CCC2(C)c2ncc(-c3ccc(C(=O)O)cc3)cc2F)cc1 ; "Chiral-HPLC" ; CC(C)c1ccc(NC(=O)N2CC[C@@]2(C)c2ncc(-c3ccc(C(=O)O)cc3)cc2F)cc1 ; CC(C)c1ccc(NC(=O)N2CC[C@]2(C)c2ncc(-c3ccc(C(=O)O)cc3)cc2F)cc1]

4-(5-Fluoro-6-(1-((4-isopropylphenyl)carbamoyl)-2-methylazetidin-2-yl)pyridin-3-yl)benzoic acid (60.0 mg, 0.134 mmol) was resolved by prep chiral-HPLC (conditions: column: CHIRALPAK ID, 3*25 cm, 5 μm; mobile phase A: hexane (0.1% formic acid); mobile phase B: ethanol; flow rate: 40 mL/min; gradient (B %): isocratic 50% B; detector: 220/254 nm; RT 1 (min): 7.0; RT 2 (min): 15.3) to afford (Compound 97a; Method AG, Peak 1, 0.90 min) (S)-4-(5-fluoro-6-(1-((4-isopropylphenyl)carbamoyl)-2-methylazetidin-2-yl)pyridin-3-yl)benzoic acid (1^{st} peak, 16.0 mg, 27%) as a white solid and (Compound 97b; Method AG, Peak 2, 2.60 min) (R)-4-(5-fluoro-6-(1-((4-isopropylphenyl)carbamoyl)-2-methylazetidin-2-yl)pyridin-3-yl)benzoic acid (2^{nd} peak, 19.3 mg, 32%) as a white solid

| Cmpd # | Characterization Data |
|---|---|
| 97a | LCMS (ESI) [M + H]⁺: 448.1 |
| | ¹H NMR (400 MHz, DMSO-d₆) δ 13.15 (s, 1H), 9.80 (s, 1H), 8.99 (s, 1H), 8.25 (dd, J = 12.2, 2.0 |
| | Hz, 1H), 8.07 (d, J = 8.1 Hz, 2H), 7.99 (d, J = 8.0 Hz, 2H), 7.42 (d, J = 8.4 Hz, 2H), 7.11 (d, J = |
| | 8.4 Hz, 2H), 4.09 (q, J = 8.1 Hz, 1H), 3.73 (s, 1H), 2.82 (hept, J = 6.9 Hz, 1H), 2.77-2.59 (m, 1H), |
| | 2.40-2.27 (m, 1H), 1.80 (s, 3H), 1.17 (d, J = 6.8 Hz, 6H). |
| 97b | LCMS (ESI) [M + H]⁺: 448.1 |
| | ¹H NMR (400 MHz, DMSO-d₆) δ 13.19 (s, 1H), 9.81 (bs, 1H), 8.99 (s, 1H), 8.25 (dd, J = 12.2, 1.9 |
| | Hz, 1H), 8.07 (d, J = 8.0 Hz, 2H), 7.98 (d, J = 7.7 Hz, 2H), 7.42 (d, J = 8.5 Hz, 2H), 7.11 (d, J = |
| | 8.3 Hz, 2H), 4.09 (q, J = 8.0 Hz, 1H), 3.73 (s, 1H), 2.82 (hept, J = 6.9 Hz, 1H), 2.76-2.64 (m, 1H), |
| | 2.39-2.26 (m, 1H), 1.80 (s, 3H), 1.17 (d, J = 6.9 Hz, 6H). |

### Example 98: 5-(4-(1-((4-isopropylphenyl)carbamoyl)-2-methylazetidine-2-carboxamido)phenyl)-6-methylpicolinic acid

### Step 1: methyl 5-(4-(1-(tert-butoxycarbonyl)-2-methylazetidine-2-carboxamido)phenyl)-6-methylpicolinate

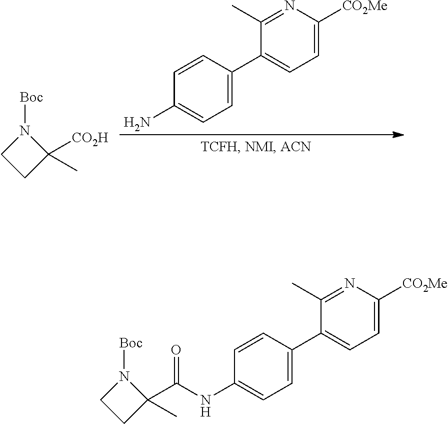 [SMILES: CC(C)(C)OC(=O)N1CCC1(C)C(=O)O ; COC(=O)c1ccc(-c2ccc(N)cc2)c(C)n1 ; COC(=O)c1ccc(-c2ccc(NC(=O)C3(C)CCN3C(=O)OC(C)(C)C)cc2)c(C)n1]

To a solution of methyl 5-(4-aminophenyl)-6-methylpyridine-2-carboxylate (300 mg, 1.24 mmol) and 1-(tert-butoxycarbonyl)-2-methylazetidine-2-carboxylic acid (400 mg, 1.86 mmol) in acetonitrile (5.0 mL) at room temperature was added 1-methylimidazole (610 mg, 7.43 mmol) and N,N,N′,N′-Tetramethylchloroformamidinium hexafluorophosphate (521 mg, 1.86 mmol). The resultant mixture was stirred for 1 hour. Upon completion, the mixture was concentrated under reduced pressure. The crude residue was directly purified by silica gel chromatography, eluted with methanol/dichloromethane (1:50) to afford methyl 5-(4-(1-(tert-butoxycarbonyl)-2-methylazetidine-2-carboxamido)phenyl)-6-methylpicolinate (450 mg, 83%) as an orange solid. LCMS (ESI) [M+H]⁺: 440.5

### Step 2: methyl 6-methyl-5-(4-(2-methylazetidine-2-carboxamido)phenyl)picolinate

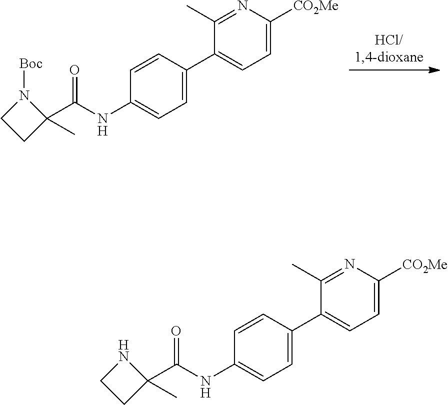 [SMILES: COC(=O)c1ccc(-c2ccc(NC(=O)C3(C)CCN3C(=O)OC(C)(C)C)cc2)c(C)n1 ; COC(=O)c1ccc(-c2ccc(NC(=O)C3(C)CCN3)cc2)c(C)n1]

A solution of methyl 5-(4-(1-(tert-butoxycarbonyl)-2-methylazetidine-2-carboxamido)phenyl)-6-methylpicolinate (450 mg, 1.02 mmol) in HCl (8 mL, 32 mmol, 4 M in 1,4-dioxane) was stirred at room temperature for 2 hours. Upon completion, the mixture was concentrated under reduced pressure. The crude residue was dissolved in dichloromethane (1.0 mL) and the solution was poured into Et₂O (20 mL). The resultant precipitate was collected by filtration, washed with Et₂O (10 mL), and dried under vacuum to afford methyl 6-methyl-5-(4-(2-methylazetidine-2-carboxamido)phenyl)picolinate (320 mg, 92%) as a light brown solid. LCMS (ESI) [M+H]⁺: 340.4

### Step 3: methyl 5-(4-(1-((4-isopropylphenyl)carbamoyl)-2-methylazetidine-2-carboxamido)phenyl)-6-methylpicolinate

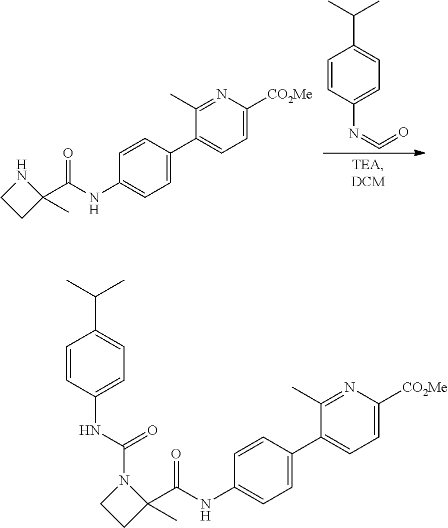 [SMILES: COC(=O)c1ccc(-c2ccc(NC(=O)C3(C)CCN3)cc2)c(C)n1 ; CC(C)c1ccc(N=C=O)cc1 ; COC(=O)c1ccc(-c2ccc(NC(=O)C3(C)CCN3C(=O)Nc3ccc(C(C)C)cc3)cc2)c(C)n1]

A solution of methyl 6-methyl-5-(4-(2-methylazetidine-2-carboxamido)phenyl)picolinate (300 mg, 0.884 mmol), 1-isocyanato-4-isopropylbenzene (285 mg, 1.77 mmol) and triethylamine (358 mg, 3.54 mmol) in dichloromethane (5.0 mL) was stirred at room temperature for 1 hour. Upon completion, the mixture was concentrated under reduced pressure. The crude residue was purified by silica gel column chromatography, eluted with petroleum ether/ethyl acetate (4:1), to afford methyl 5-(4-(1-((4-isopropylphenyl)carbamoyl)-2-methylazetidine-2-carboxamido)phenyl)-6-methylpicolinate (310 mg, 70%) as a yellow solid. LCMS (ESI) [M+H]⁺: 501.6

### Step 4: 5-(4-(1-((4-isopropylphenyl)carbamoyl)-2-methylazetidine-2-carboxamido)phenyl)-6-methylpicolinic acid

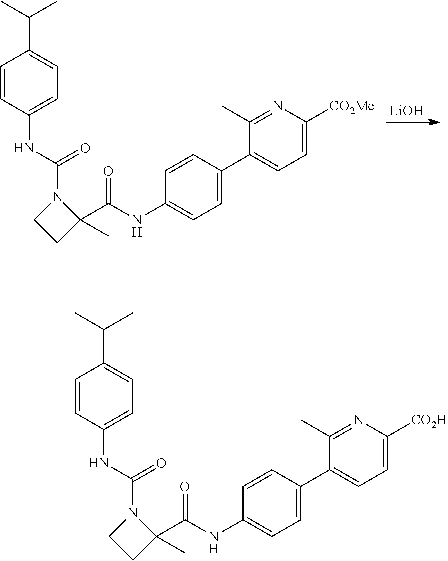

A mixture of methyl 5-(4-(1-((4-isopropylphenyl)carbamoyl)-2-methylazetidine-2-carboxamido)phenyl)-6-methylpicolinate (310 mg, 0.619 mmol), and lithium hydroxide monohydrate (130 mg, 3.09 mmol) in tetrahydrofuran (1.5 mL), methanol (1.5 mL) and water (1.5 mL) was stirred at room temperature for 1 hour. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 10% to 70% over 30 min; detector: UV 254 nm) to afford crude 5-(4-(1-((4-isopropylphenyl)carbamoyl)-2-methylazetidine-2-carboxamido)phenyl)-6-methylpicolinic acid (170 mg, 56%, 90% purity) as a pale-yellow solid. A portion of the residue (60 mg, 0.123 mmol) was purified again by reversed-phase flash chromatography (conditions: column: C18 silica gel; mobile phase A: water (0.1% formic acid); mobile phase B: acetonitrile; gradient (B %): 10% to 80% over 20 min; detector: UV 254 nm) to afford (Compound 98) 5-(4-(1-((4-isopropylphenyl)carbamoyl)-2-methylazetidine-2-carboxamido)phenyl)-6-methylpicolinic acid (17.8 mg, 30%) as a white solid.

| Cmpd # | Characterization Data |
|---|---|
| 98 | LCMS (ESI) [M + H]⁺: 487.2 |
| | ¹H NMR (300 MHz, DMSO-d₆) δ 10.75 (s, 1H), 8.62 (s, 1H), 7.94 (d, J = 7.8 Hz, 1H), 7.74 (dd, J = |
| | 16.2, 8.2 Hz, 3H), 7.45 (d, J = 7.8 Hz, 4H), 7.15 (d, J = 8.2 Hz, 2H), 4.06 (d, J = 8.0 Hz, 1H), |
| | 3.89 (s, 1H), 3.09-3.54 (s, 1H), 2.84 (s, 1H), 2.64 (s, 2H), 2.39 (s, 1H), 2.09 (s, 1H), 1.74 (s, 3H), |
| | 1.18 (d, J = 6.9 Hz, 6H). |

### Example 99: (R)-4-(5-fluoro-6-(1-((5-fluoro-6-(1-(trifluoromethyl)cyclopropyl)pyridin-3-yl)carbamoyl)pyrrolidin-2-yl)pyridin-3-yl)benzoic acid

### Step 1: methyl 5-fluoro-6-(3,3,3-trifluoroprop-1-en-2-yl)nicotinate

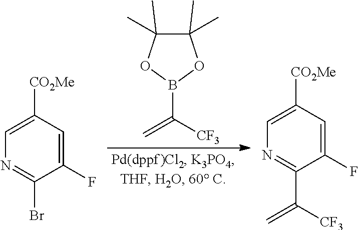

A solution of methyl 6-bromo-5-fluoropyridine-3-carboxylate (3.00 g, 12.8 mmol), 4,4,5,5-tetramethyl-2-(3,3,3-trifluoroprop-1-en-2-yl)-1,3,2-dioxaborolane (5.69 g, 25.6 mmol), K₃PO₄ (8.16 g, 38.4 mmol) and Pd(dppf)Cl₂ (940 mg, 1.28 mmol) in THF (30.0 mL) and water (10.0 mL) was stirred at 80° C. for 1 hour under nitrogen atmosphere. Upon completion, the mixture was concentrated under reduced pressure. The crude residue was purified by silica gel column chromatography, eluted with petroleum ether/ethyl acetate (1:1), to afford methyl 5-fluoro-6-(3,3,3-trifluoroprop-1-en-2-yl)nicotinate (2.00 g, 63% yield, 63% purity) as a yellow oil. LCMS (ESI) [M+H]⁺: 250.2

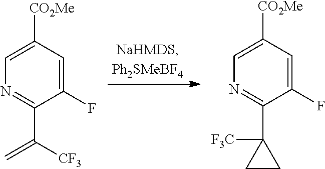

To a solution of diphenyl(methyl)sulfonium tetrafluoroborate (301 mg, 1.04 mmol) in THF (3.0 mL) at 0° C. was added NaHMDS (1.0 mL, 2.0 mmol, 2 M in THF) followed by stirring for 5 minutes. Then, methyl 5-fluoro-6-(3,3,3-trifluoroprop-1-en-2-yl)nicotinate (200 mg, 0.803 mmol) was added. The mixture was warmed to room temperature and stirred for 1 hour. Upon completion, the reaction was quenched with water (20 mL) and extracted with ethyl acetate (3×30 mL). The combined organic layers were the concentrated under reduced pressure. The crude residue was purified by silica gel column chromatography, eluted with petroleum ether/ethyl acetate (5:1) to afford methyl 5-fluoro-6-(1-(trifluoromethyl)cyclopropyl)nicotinate (60.0 mg, 28%) as a yellow oil. LCMS (ESI) [M+H]⁺: 264.2

### Step 3: 5-fluoro-6-(1-(trifluoromethyl)cyclopropyl)nicotinic acid

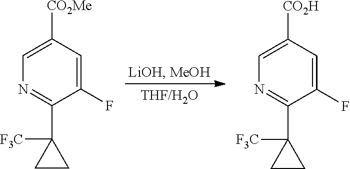

To a solution of methyl 5-fluoro-6-(1-(trifluoromethyl)cyclopropyl)nicotinate (60.0 mg, 0.228 mmol) in methanol (1.0 mL) and THF (1.0 mL) was added a solution of lithium hydroxide monohydrate (47.8 mg, 1.14 mmol) in water (0.50 mL), and the resultant mixture was stirred at room temperature for 1 hour. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 10% to 50% over 10 min; detector: UV 254 nm) to afford 5-fluoro-6-(1-(trifluoromethyl)cyclopropyl)nicotinic acid (50.0 mg, 88%) as a white solid. LCMS (ESI) [M+H]⁺: 250.2

### Step 4: methyl (R)-4-(5-fluoro-6-(1-((5-fluoro-6-(1-(trifluoromethyl)cyclopropyl)pyridin-3-yl)carbamoyl)pyrrolidin-2-yl)pyridin-3-yl)benzoate

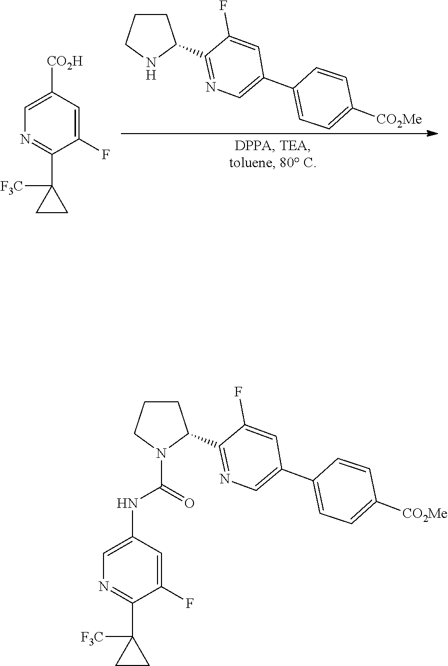 [SMILES: O=C(O)c1cnc(C2(C(F)(F)F)CC2)c(F)c1 ; COC(=O)c1ccc(-c2cnc([C@H]3CCCN3)c(F)c2)cc1 ; COC(=O)c1ccc(-c2cnc([C@H]3CCCN3C(=O)Nc3cnc(C4(C(F)(F)F)CC4)c(F)c3)c(F)c2)cc1]

A solution of methyl (R)-4-(5-fluoro-6-(pyrrolidin-2-yl)pyridin-3-yl)benzoate (72.3 mg, 0.241 mmol), DPPA (82.8 mg, 0.301 mmol), triethylamine (60.9 mg, 0.603 mmol), and 5-fluoro-6-(1-(trifluoromethyl)cyclopropyl)nicotinic acid (50.0 mg, 0.201 mmol) in toluene (3.0 mL) was stirred at 80° C. for 1 hour under nitrogen atmosphere. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 10% to 50% over 10 min; detector: UV 254 nm) to afford methyl (R)-4-(5-fluoro-6-(1-((5-fluoro-6-(1-(trifluoromethyl)cyclopropyl)pyridin-3-yl)carbamoyl)pyrrolidin-2-yl)pyridin-3-yl)benzoate (30 mg, 27%) as a yellow solid. LCMS (ESI) [M+H]⁺: 547.5

### Step 5: (R)-4-(5-fluoro-6-(1-((5-fluoro-6-(1-(trifluoromethyl)cyclopropyl)pyridin-3-yl)carbamoyl)pyrrolidin-2-yl)pyridin-3-yl)benzoic acid

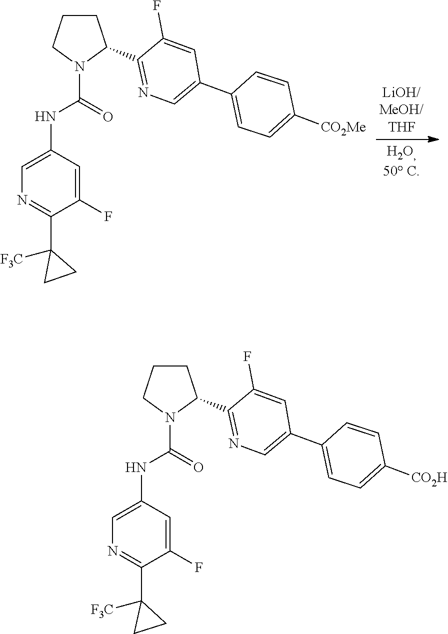 [SMILES: COC(=O)c1ccc(-c2cnc([C@H]3CCCN3C(=O)Nc3cnc(C4(C(F)(F)F)CC4)c(F)c3)c(F)c2)cc1 ; O=C(O)c1ccc(-c2cnc([C@H]3CCCN3C(=O)Nc3cnc(C4(C(F)(F)F)CC4)c(F)c3)c(F)c2)cc1]

To a solution of methyl (R)-4-(5-fluoro-6-(1-((5-fluoro-6-(1-(trifluoromethyl)cyclopropyl)pyridin-3-yl)carbamoyl)pyrrolidin-2-yl)pyridin-3-yl)benzoate (30.0 mg, 0.055 mmol) in THF (1.0 mL) and methanol (1.0 mL) was added a solution of lithium hydroxide monohydrate (11.5 mg, 0.275 mmol) in water (0.50 mL), and the resultant mixture was stirred at 50° C. for 1 hour. Upon completion, the mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 10% to 50% over 10 min; detector: UV 254 nm) to afford (R)-4-(5-fluoro-6-(1-((5-fluoro-6-(1-(trifluoromethyl)cyclopropyl)pyridin-3-yl)carbamoyl)pyrrolidin-2-yl)pyridin-3-yl)benzoic acid (16.3 mg, 56%) as a white solid.

| Cmpd # | Characterization Data |
|---|---|
| 99 | LCMS (ESI) [M + H]⁺: 533.1 |
| | ¹H NMR (400 MHz, DMSO) δ 8.87 (s, 1H), 8.73 (d, J = 1.8 Hz, 1H), 8.49 (s, 1H), 8.14-7.99 (m, |
| | 3H), 7.95-7.85 (m, 3H), 5.37 (d, J = 5.5 Hz, 1H), 3.80-3-77 (m, J = 8.2 Hz, 1H), 3.67 (d, J = 8.2 |
| | Hz, 1H), 2.40-3.36 (m, J = 12.3, 8.0 Hz, 1H), 2.18-1.86 (m, 4H), 1.41-1.35 (m, J = 4.8 Hz, 2H), |
| | 1.15 (s, 2H). |

### Example 102: (R)-5-(4-(1-((4-isopropylphenyl)carbamoyl)-3,3-dimethylazetidine-2-carboxamido)phenyl)-6-methylpicolinic acid

### Step 1: methyl (R)-3,3-dimethyl-2-(picolinamido)butanoate

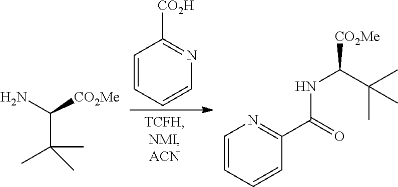

A solution of picolinic acid (1.02 g, 8.26 mmol), methyl (2R)-2-amino-3,3-dimethylbutanoate (1.00 g, 6.89 mmol), TCFH (3.86 g, 13.8 mmol) and NMI (2.83 g, 34.4 mmol) in acetonitrile (10.0 mL) was stirred at room temperature for 1 hour. Upon completion, the mixture was concentrated under reduced pressure. The crude residue was purified by silica gel column chromatography, eluted with petroleum ether/ethyl acetate (1:1) to afford methyl (2R)-3,3-dimethyl-2-(pyridin-2-ylformamido)butanoate (1.00 g, 58%) as a yellow oil. LCMS (ESI) [M+H]⁺: 251.3

### Step 2: Methyl (R)-3,3-dimethyl-1-picolinoylazetidine-2-carboxylate

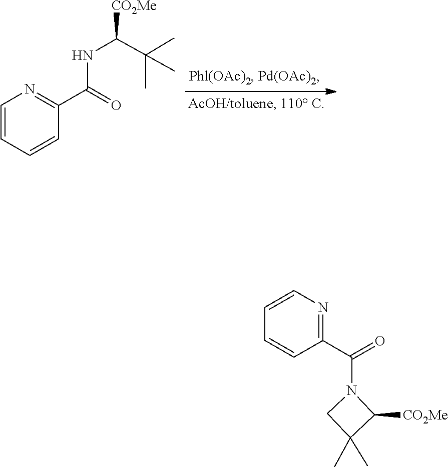 [SMILES: COC(=O)[C@H](NC(=O)c1ccccn1)C(C)(C)C ; COC(=O)[C@@H]1N(C(=O)c2ccccn2)CC1(C)C]

A solution of methyl (2R)-3,3-dimethyl-2-(pyridin-2-ylformamido)butanoate (1.00 g, 3.99 mmol), PhI(OAc)₂ (3.22 g, 9.99 mmol), acetic acid (0.48 g, 7.990 mmol) and Pd(OAc)₂ (0.04 g, 0.200 mmol) in toluene (10 mL) was stirred at 110° C. for 1 hour under nitrogen atmosphere. Upon completion, the mixture was concentrated under reduced pressure. The crude residue was directly purified by silica gel column chromatography, eluted with petroleum ether/ethyl acetate (1:1) to afford methyl (R)-3,3-dimethyl-1-picolinoylazetidine-2-carboxylate (600 mg, 61%) as a yellow oil. LCMS (ESI) [M+H]⁺: 249.2

### Step 3: (R)-3,3-dimethylazetidine-2-carboxylic acid

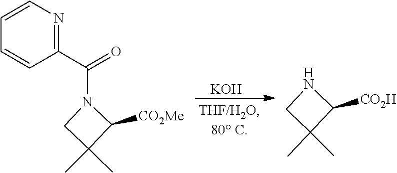

A solution of methyl (R)-3,3-dimethyl-1-picolinoylazetidine-2-carboxylate (300 mg, 1.21 mmol) and KOH (678 mg, 12.1 mmol) in methanol (2.0 mL), THF (2.0 mL) and water (1.0 mL) was stirred at 80° C. for 1 hour. Upon completion, the mixture was concentrated under reduced pressure. The crude residue was stirred with dichloromethane (20 mL). The mixture was then filtered, and the filtrate was concentrated to give crude (2R)-3,3-dimethylazetidine-2-carboxylic acid (100 mg, crude) as a white solid, which was used in the next step without further purification. LCMS (ESI) [M+H]⁺: 130.0

### Step 4: (R)-1-((4-isopropylphenyl)carbamoyl)-3,3-dimethylazetidine-2-carboxylic acid

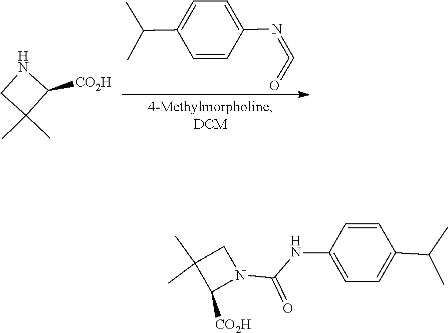

A solution of (2R)-3,3-dimethylazetidine-2-carboxylic acid (100 mg, 0.774 mmol), N-methylmorpholine (235 mg, 2.32 mmol) and 1-isocyanato-4-isopropylbenzene (187 mg, 1.16 mmol) in dichloromethane (5.0 mL) was stirred at room temperature for 1 hour under nitrogen atmosphere. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 10% to 60% over 10 min; detector: UV 254 nm) to afford (R)-1-((4-isopropylphenyl)carbamoyl)-3,3-dimethylazetidine-2-carboxylic acid (100 mg, 45%) as a pink solid. LCMS (ESI) [M+H]⁺: 291.1

### Step 5: methyl (R)-5-(4-(1-((4-isopropylphenyl)carbamoyl)-3,3-dimethylazetidine-2-carboxamido)phenyl)-6-methylpicolinate

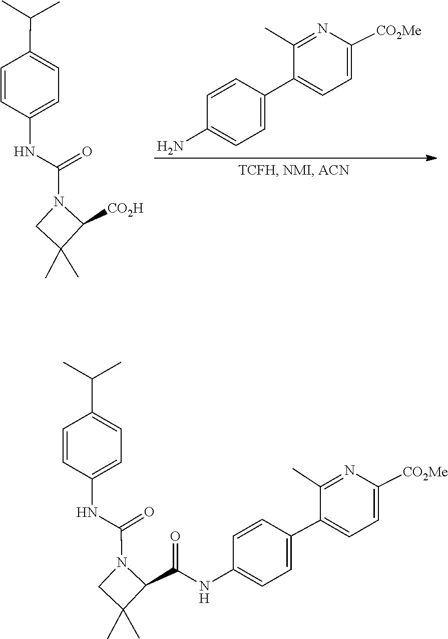 [SMILES: CC(C)c1ccc(NC(=O)N2CC(C)(C)[C@@H]2C(=O)O)cc1 ; COC(=O)c1ccc(-c2ccc(N)cc2)c(C)n1 ; COC(=O)c1ccc(-c2ccc(NC(=O)[C@@H]3N(C(=O)Nc4ccc(C(C)C)cc4)CC3(C)C)cc2)c(C)n1]

A solution of methyl 5-(4-aminophenyl)-6-methylpyridine-2-carboxylate (83.4 mg, 0.344 mmol), (2R)-1-[(4-isopropylphenyl)carbamoyl]-3,3-dimethylazetidine-2-carboxylic acid (100 mg, 0.344 mmol), TCFH (193 mg, 0.688 mmol) and NMI (141 mg, 1.72 mmol) in acetonitrile (5.0 mL) was stirred at room temperature for 1 hour. Upon completion, the mixture was concentrated under reduced pressure. The crude residue was purified by silica gel column chromatography, eluted with petroleum ether/ethyl acetate (1:1), to afford methyl (R)-5-(4-(1-((4-isopropylphenyl)carbamoyl)-3,3-dimethylazetidine-2-carboxamido)phenyl)-6-methylpicolinate (120 mg, 68%) as a yellow-green solid. LCMS (ESI) [M+H]⁺: 515.2

### Step 6: (R)-5-(4-(1-((4-isopropylphenyl)carbamoyl)-3,3-dimethylazetidine-2-carboxamido)phenyl)-6-methylpicolinic acid

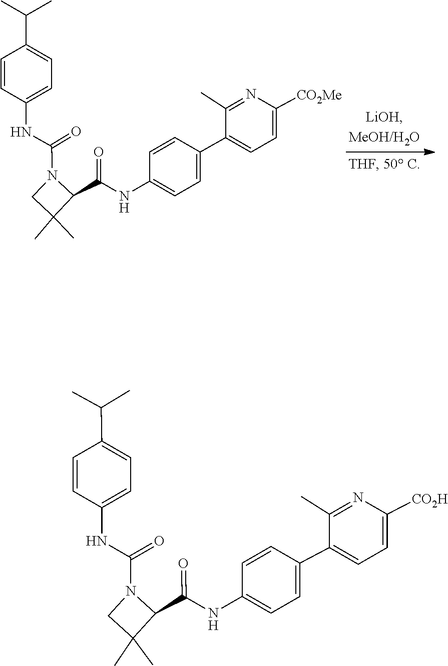 [SMILES: COC(=O)c1ccc(-c2ccc(NC(=O)[C@@H]3N(C(=O)Nc4ccc(C(C)C)cc4)CC3(C)C)cc2)c(C)n1 ; Cc1nc(C(=O)O)ccc1-c1ccc(NC(=O)[C@@H]2N(C(=O)Nc3ccc(C(C)C)cc3)CC2(C)C)cc1]

A solution of methyl (R)-5-(4-(1-((4-isopropylphenyl)carbamoyl)-3,3-dimethylazetidine-2-carboxamido)phenyl)-6-methylpicolinate (120 mg, 0.233 mmol) and lithium hydroxide monohydrate (48.7 mg, 1.16 mmol) in methanol (2.0 mL), THF (2.0 mL) and water (1.0 mL) was stirred at 50° C. for 1 hour. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 10% to 80% over 18 min; detector: UV 254 nm) to afford (Compound 102; Method E, Peak 1, 1.65 min—analyzed against Compound 103) (R)-5-(4-(1-((4-isopropylphenyl)carbamoyl)-3,3-dimethylazetidine-2-carboxamido)phenyl)-6-methylpicolinic acid (51.5 mg, 44%) as a white solid.

| Cmpd # | Characterization Data |
|---|---|
| 102 | LCMS (ESI) [M + H]⁺: 501.2 |
| | ¹H NMR (400 MHz, DMSO) δ 10.00 (s, 1H), 8.45 (s, 1H), 7.93 (d, J = 7.8 Hz, 1H), 7.83-7.72 |
| | (m, 3H), 7.44-7.35 (m, 4H), 7.14-7.04 (m, 2H), 4.41 (s, 1H), 3.76 (d, J = 7.3 Hz, 1H), 3.66 (d, |
| | J = 7.3 Hz, 1H), 2.85-2.76 (m, J = 6.9 Hz, 1H), 2.52 (s, 3H), 1.40 (s, 3H), 1.19-1.10 (m, 9H). |

### Example 103: (S)-5-(4-(1-((4-isopropylphenyl)carbamoyl)-3,3-dimethylazetidine-2-carboxamido)phenyl)-6-methylpicolinic acid

### Step 1: methyl (S)-3,3-dimethyl-2-(picolinamido)butanoate

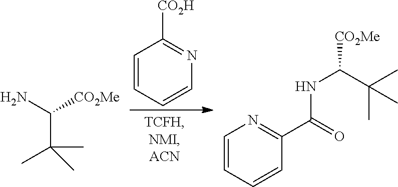

A solution of picolinic acid (2.03 g, 16.5 mmol), methyl (S)-2-amino-3,3-dimethylbutanoate (2.00 g, 13.8 mmol), TCFH (7.73 g, 27.5 mmol) and NMI (5.65 g, 68.9 mmol) in acetonitrile (20.0 mL) was stirred at room temperature for 1 hour. Upon completion, the resultant mixture was concentrated under reduced pressure. The crude residue was directly purified by silica gel column chromatography, eluted with petroleum ether/ethyl acetate (1:1), to afford methyl (S)-3,3-dimethyl-2-(picolinamido)butanoate (2.00 g, 58%) as a yellow oil. LCMS (ESI) [M+H]⁺: 251.3

### Step 2: methyl (S)-3,3-dimethyl-1-picolinoylazetidine-2-carboxylate

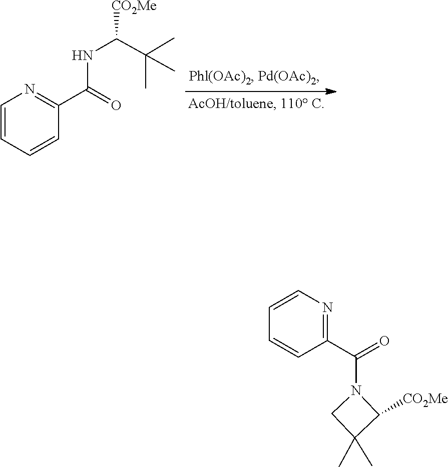 [SMILES: COC(=O)[C@@H](NC(=O)c1ccccn1)C(C)(C)C ; COC(=O)[C@H]1N(C(=O)c2ccccn2)CC1(C)C]

A solution of methyl (S)-3,3-dimethyl-2-(picolinamido)butanoate (500 mg, 1.99 mmol) and PhI(OAc)₂ (1.60 g, 4.99 mmol), acetic acid (240 mg, 3.99 mmol) and Pd(OAc)₂ (22.4 mg, 0.100 mmol) in toluene (10.0 mL) was stirred at 110° C. for 1 hour under nitrogen atmosphere. Upon completion, the mixture was concentrated under reduced pressure. The crude residue was purified by silica gel column chromatography, eluted with petroleum ether/ethyl acetate (1:1), to afford methyl (S)-3,3-dimethyl-1-picolinoylazetidine-2-carboxylate (400 mg, 81%) as a yellow oil. LCMS (ESI) [M+H]⁺: 249.2

### Step 3: (S)-3,3-dimethylazetidine-2-carboxylic acid

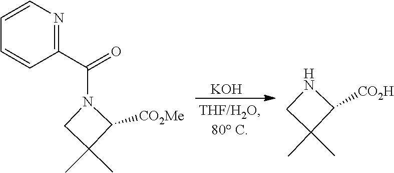

A solution of methyl (S)-3,3-dimethyl-1-picolinoylazetidine-2-carboxylate (400 mg, 1.61 mmol) and KOH (904 mg, 16.1 mmol) in methanol (2.0 mL), THF (2.0 mL), and water (1.0 mL) was stirred at 80° C. for 1 hour. Upon completion, the mixture was concentrated under reduced pressure. The crude residue was stirred with dichloromethane (20 mL). The mixture was then filtered, and the filtrate was concentrated to afford crude (S)-3,3-dimethylazetidine-2-carboxylic acid (200 mg, crude) as a white solid, which was used in the next step without further purification. LCMS (ESI) [M+H]⁺: 130.0

### Step 4: (S)-1-((4-isopropylphenyl)carbamoyl)-3,3-dimethylazetidine-2-carboxylic acid

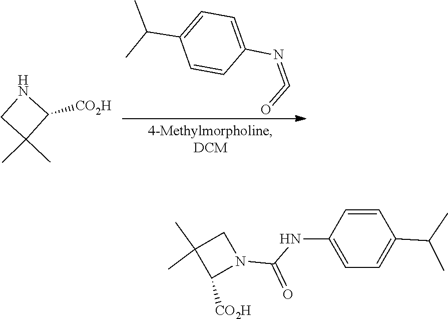

A solution of (S)-3,3-dimethylazetidine-2-carboxylic acid (200 mg, 1.55 mmol), N-methylmorpholine (470 mg, 4.65 mmol) and 1-isocyanato-4-isopropylbenzene (375 mg, 2.33 mmol) in dichloromethane (5.0 mL) was stirred at room temperature for 1 hour under nitrogen atmosphere. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 10% to 60% over 10 min; detector: UV 254 nm) to afford (S)-1-((4-isopropylphenyl)carbamoyl)-3,3-dimethylazetidine-2-carboxylic acid (200 mg, 45%) as a yellow solid. LCMS (ESI) [M+H]⁺: 291.1

### Step 5: Methyl (S)-5-(4-(1-((4-isopropylphenyl)carbamoyl)-3,3-dimethylazetidine-2-carboxamido)phenyl)-6-methylpicolinate

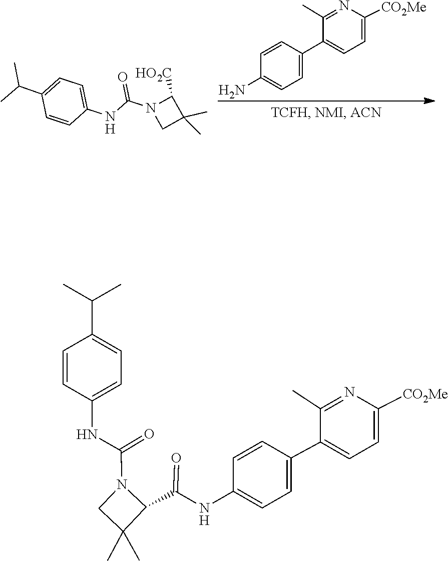

A solution of methyl 5-(4-aminophenyl)-6-methylpyridine-2-carboxylate (80.0 mg, 0.330 mmol), (S)-1-((4-isopropylphenyl)carbamoyl)-3,3-dimethylazetidine-2-carboxylic acid (144 mg, 0.495 mmol), TCFH (185 mg, 0.660 mmol) and NMI (136 mg, 1.65 mmol) in acetonitrile (5.0 mL) was stirred at room temperature for 1 hour under nitrogen atmosphere. Upon completion, the mixture was concentrated under reduced pressure. The crude residue was directly purified by silica gel column chromatography, eluted with petroleum ether/ethyl acetate (1:1), to afford methyl (S)-5-(4-(1-((4-isopropylphenyl)carbamoyl)-3,3-dimethylazetidine-2-carboxamido)phenyl)-6-methylpicolinate (70 mg, 41%) as a yellow solid. LCMS (ESI) [M+H]⁺: 515.6

### Step 6: (S)-5-(4-(1-((4-isopropylphenyl)carbamoyl)-3,3-dimethylazetidine-2-carboxamido)phenyl)-6-methylpicolinic acid

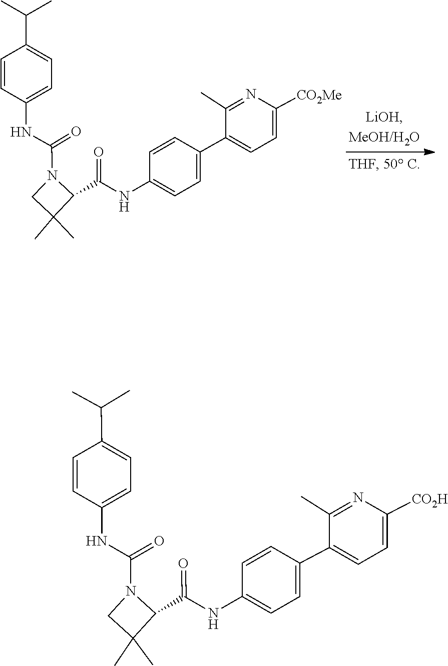 [SMILES: COC(=O)c1ccc(-c2ccc(NC(=O)[C@H]3N(C(=O)Nc4ccc(C(C)C)cc4)CC3(C)C)cc2)c(C)n1 ; Cc1nc(C(=O)O)ccc1-c1ccc(NC(=O)[C@H]2N(C(=O)Nc3ccc(C(C)C)cc3)CC2(C)C)cc1]

A solution of methyl (S)-5-(4-(1-((4-isopropylphenyl)carbamoyl)-3,3-dimethylazetidine-2-carboxamido)phenyl)-6-methylpicolinate (70 mg, 0.136 mmol) and lithium hydroxide monohydrate (28.6 mg, 0.680 mmol) in methanol (2.0 mL), THF (2.0 mL) and water (1.0 mL) was stirred at 50° C. for 1 hour. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 10% to 70% over 20 min; detector: UV 254 nm) to afford (Compound 103; Method E, Peak 2, 2.10 min—analyzed against Compound 102) (S)-5-(4-(1-((4-isopropylphenyl)carbamoyl)-3,3-dimethylazetidine-2-carboxamido)phenyl)-6-methylpicolinic acid (32.2 mg, 47%) as a white solid.

| Cmpd # | Characterization Data |
|---|---|
| 103 | LCMS (ESI) [M + H]⁺: 501.2 |
| | ¹H NMR (400 MHz, DMSO) δ 10.00 (s, 1H), 8.45 (s, 1H), 7.93 (d, J = 7.9 Hz, 1H), 7.82-7.73 (m, |
| | J = 8.3, 7.0 Hz, 3H), 7.45-7.33 (m, 4H), 7.15-7.01 (m, 2H), 4.41 (s, 1H), 3.76 (d, J = 7.3 Hz, |
| | 1H), 3.66 (d, J = 7.3 Hz, 1H), 2.81 (p, J = 6.9 Hz, 1H), 2.52 (s, 3H), 1.40 (s, 3H), 1.22-1.07 (m, |
| | 9H). |

### Example 104: (R)-5-(4-(4-((4-isopropylphenyl)carbamoyl)-2,2-dimethylazetidine-1-carboxamido)phenyl)-6-methylpicolinic acid

### Step 1: tert-butyl (R)-4-((4-isopropylphenyl)carbamoyl)-2,2-dimethylazetidine-1-carboxylate

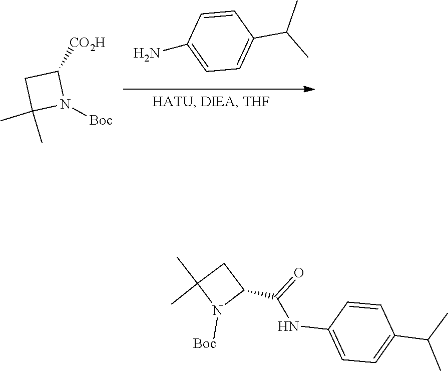

A solution of (R)-1-(tert-butoxycarbonyl)-4,4-dimethylazetidine-2-carboxylic acid (100 mg, 0.436 mmol), 4-isopropylaniline (65 mg, 0.480 mmol), HATU (249 mg, 0.654 mmol) and diisopropylethylamine (169 mg, 1.31 mmol) in THF (5.0 mL) was stirred at room temperature for 2 hours. Upon completion, the mixture was concentrated under reduced pressure. The crude residue was purified by reversed-phase flash chromatography (conditions: column: C18 silica gel; mobile phase A: water (10 mM NH₄HCO₃), mobile phase B: acetonitrile; gradient (B %): 0% to 100% over 20 min; detector: UV 254 nm) to afford tert-butyl (R)-4-((4-isopropylphenyl)carbamoyl)-2,2-dimethylazetidine-1-carboxylate (110 mg, 73% yield) as a white solid. LCMS (ESI) [M+H]⁺: 347.2

### Step 2: (R)—N-(4-isopropylphenyl)-4,4-dimethylazetidine-2-carboxamide

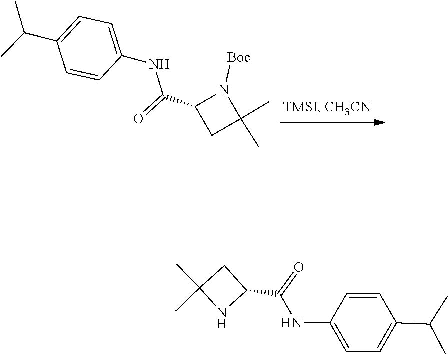

To a mixture of tert-butyl (R)-4-((4-isopropylphenyl)carbamoyl)-2,2-dimethylazetidine-1-carboxylate (80 mg, 0.231 mmol) in acetonitrile (3.0 mL) at 0° C. was added trimethylsilyl iodide (139 mg, 0.693 mmol) dropwise. The resultant mixture was warmed to room temperature and stirred for 1 hour. Upon completion, the mixture was concentrated under reduced pressure. The crude residue was directly purified by reversed-phase flash chromatography (conditions: column: C18 silica gel; mobile phase A: water (10 mM NH₄HCO₃), mobile phase B: acetonitrile; gradient (B %): 0-100% over 20 min; detector: UV 254 nm) to afford (R)—N-(4-isopropylphenyl)-4,4-dimethylazetidine-2-carboxamide (35 mg, 62% yield) as a white solid. LCMS (ESI) [M+H]⁺: 247.2

### Step 3: methyl (R)-5-(4-(4-((4-isopropylphenyl)carbamoyl)-2,2-dimethylazetidine-1-carboxamido)phenyl)-6-methylpicolinate

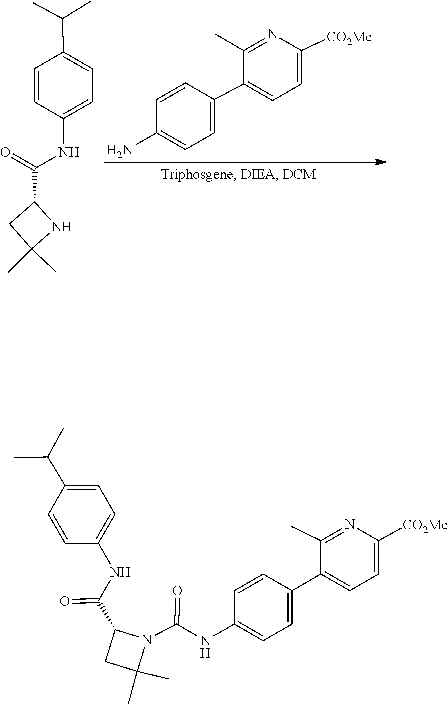 [SMILES: CC(C)c1ccc(NC(=O)[C@H]2CC(C)(C)N2)cc1 ; COC(=O)c1ccc(-c2ccc(N)cc2)c(C)n1 ; COC(=O)c1ccc(-c2ccc(NC(=O)N3[C@@H](C(=O)Nc4ccc(C(C)C)cc4)CC3(C)C)cc2)c(C)n1]

To a mixture of methyl 5-(4-aminophenyl)-6-methylpicolinate (30.0 mg, 0.122 mmol) and diisopropylethylamine (79.0 mg, 0.610 mmol) in dichloromethane (1.0 mL) at 0° C. was added triphosgene (14.0 mg, 49.0 mmol). After 30 minutes, the reaction was warmed to room temperature and (R)—N-(4-isopropylphenyl)-4,4-dimethylazetidine-2-carboxamide (30.0 mg, 0.122 mmol) was added dropwise over 1 minute. The reaction was allowed to stir for an additional 2 hours. Upon completion, the mixture was cooled to 0° C. and quenched with ice water. The mixture was extracted with ethyl acetate (3×2 mL). The combined organic layers were dried over anhydrous sodium sulfate, filtered and concentrated under reduced pressure. The crude residue was purified by silica gel column chromatography, eluted with petroleum ether/ethyl acetate (1:1), to afford methyl (R)-5-(4-(4-((4-isopropylphenyl)carbamoyl)-2,2-dimethylazetidine-1-carboxamido)phenyl)-6-methylpicolinate (28 mg, 45%) as a white solid. LCMS (ESI) [M+H]⁺: 515.3

### Step 4: (R)-5-(4-(4-((4-isopropylphenyl)carbamoyl)-2,2-dimethylazetidine-1-carboxamido)phenyl)-6-methylpicolinic acid

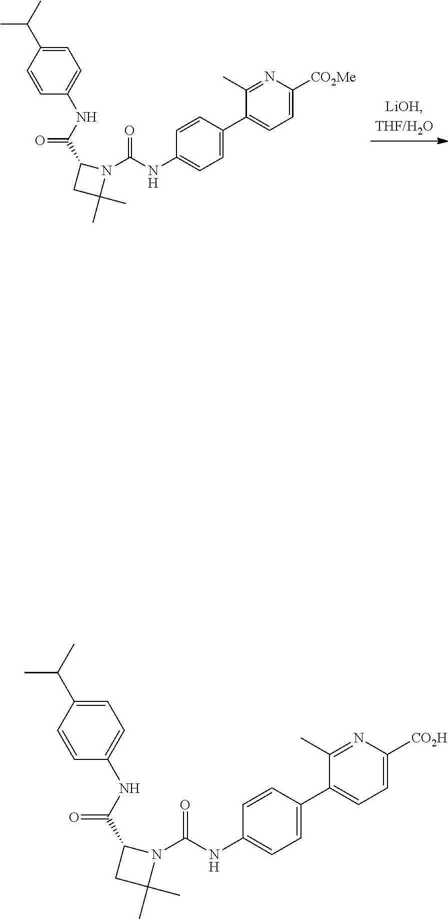 [SMILES: COC(=O)c1ccc(-c2ccc(NC(=O)N3[C@@H](C(=O)Nc4ccc(C(C)C)cc4)CC3(C)C)cc2)c(C)n1 ; Cc1nc(C(=O)O)ccc1-c1ccc(NC(=O)N2[C@@H](C(=O)Nc3ccc(C(C)C)cc3)CC2(C)C)cc1]

To a mixture of methyl (R)-5-(4-(4-((4-isopropylphenyl)carbamoyl)-2,2-dimethylazetidine-1-carboxamido)phenyl)-6-methylpicolinate (23 mg, 45.0 mmol) in THF (0.50 mL) and water (0.50 mL) at 0° C. was added lithium hydroxide monohydrate (6.00 mg, 0.135 mmol). The resultant mixture was warmed to room temperature and stirred for 2 hours. Upon completion, the mixture was concentrated under reduced pressure. The crude residue was directly purified by reversed-phase flash chromatography (conditions: column: C18 silica gel; mobile phase A, water (0.1% formic acid), mobile phase B: acetonitrile; gradient (B %): 0% to 100% over 25 min; detector: UV 254/220 nm) to afford (R)-5-(4-(4-((4-isopropylphenyl)carbamoyl)-2,2-dimethylazetidine-1-carboxamido)phenyl)-6-methylpicolinic acid (4.2 mg, 19%) as a white solid.

| Cmpd # | Characterization Data |
|---|---|
| 104 | LCMS (ESI) [M + H]⁺: 501.2 |
| | ¹H NMR (400 MHz, DMSO-d₆) δ 7.93-7.68 (m, 2H), 7.59-7.53 (m, 1H), 7.32 (d, J = 8.4 Hz, |
| | 2H), 7.20 (d, J = 8.4 Hz, 2H), 4.93-4.86 (m, 1H), 2.91-2.82 (m, 1H), 2.53-2.50 (m, 3H), |
| | 2.42-2.34 (m, 2H), 2.08-1.99 (m, 1H), 1.57-1.53 (m, 6H), 1.18 (d, J = 7.2 Hz, 8H). |

### Example 105: (R)-4-(2-(1-((5-fluoro-6-(1-trifluoromethyl)cyclopropyl)pyridin-3-yl)carbamoyl)pyrrolidin-2-yl)benzo[d]oxazol-6-yl)benzoic acid

### Step 1: (R)-4-(2-(1-((5-fluoro-6-(1-(trifluoromethyl)cyclopropyl)pyridin-3-yl)carbamoyl)pyrrolidin-2-yl)benzo[d]oxazol-6-yl)benzoic acid

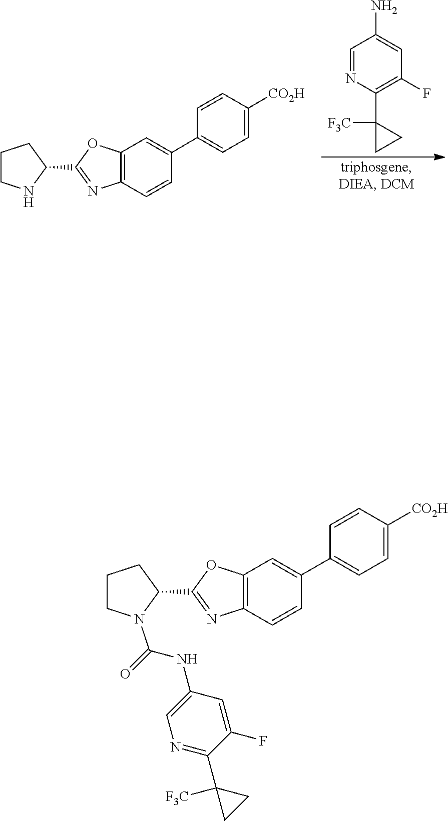 [SMILES: O=C(O)c1ccc(-c2ccc3nc([C@H]4CCCN4)oc3c2)cc1 ; Nc1cnc(C2(C(F)(F)F)CC2)c(F)c1 ; O=C(O)c1ccc(-c2ccc3nc([C@H]4CCCN4C(=O)Nc4cnc(C5(C(F)(F)F)CC5)c(F)c4)oc3c2)cc1]

To a mixture of 5-fluoro-6-(1-(trifluoromethyl)cyclopropyl)pyridin-3-amine (36.0 mg, 0.160 mmol) and diisopropylethylamine (105 mg, 0.810 mmol) in dichloromethane (5.0 mL) at 0° C. was added triphosgene (19.0 mg, 60.0 mmol). After 30 minutes, the reaction was warmed to room temperature and (R)-4-(2-(pyrrolidin-2-yl)benzo[d]oxazol-6-yl)benzoic acid (50 mg, 0.160 mmol) was added dropwise over 1 minute. The reaction was allowed to stir for an additional 2 hours. Upon completion, the mixture was cooled to 0° C. and quenched with ice water. The mixture was extracted with ethyl acetate (3×8 mL). The combined organic layers were dried over anhydrous sodium sulfate, filtered, and concentrated under reduced pressure. The crude residue was purified by preparative HPLC (conditions: column: Xselect CSH Prep C18, 30*150 mm 5 μm; mobile phase A: water (0.1% formic acid); mobile phase B: acetonitrile; gradient (B %): 47% to 67% over 10 min; detector: 254 nm/220 nm; flow rate: 60 mL/min; RT (min): 11.2) to afford (R)-4-(2-(1-((5-fluoro-6-(1-(trifluoromethyl)cyclopropyl)pyridin-3-yl)carbamoyl)pyrrolidin-2-yl)benzo[d]oxazol-6-yl)benzoic acid (7.0 mg, 8% yield) as a white solid.

| Cmpd # | Characterization Data |
|---|---|
| 105 | LCMS (ESI) [M + H]⁺: 555.2 |
| | ¹H NMR (400 MHz, DMSO-d₆) δ 9.05 (s, 1H), 8.53 (s, 1H), 8.09 (s, 1H), 8.02 (d, J = 8.4 Hz, 2H), |
| | 7.95-7.92 (m, 1H), 7.85 (d, J = 8.4 Hz, 2H), 7.79 (d, J = 8.4 Hz, 1H), 7.74 (d, J = 8.4 Hz, 1H), |
| | 5.31 (d, J = 7.6 Hz, 1H), 3.80 (s, 1H), 3.66-3.63 (m, 1H), 2.43-2.41 (m, 1H), 2.18-2.06 (m, |
| | 3H), 1.39-1.36 (m, 2H), 1.17 (s, 2H). |

### Example 106: (R)-4-(2-(1-((3-fluoro-4-(1-(trifluoromethyl)cyclopropyl)phenyl)carbamoyl)pyrrolidin-2-yl)benzo[d]oxazol-6-yl)benzoic acid

### Step 1: tert-butyl (4-bromo-3-fluorophenyl)carbamate

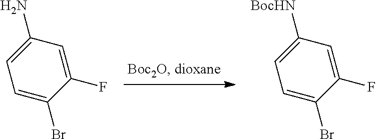 [SMILES: Nc1ccc(Br)c(F)c1 ; CC(C)(C)OC(=O)Nc1ccc(Br)c(F)c1]

A solution of 4-bromo-3-fluoroaniline (10.0 g, 52.6 mmol) and Boc₂O (23.0 g, 105 mmol) in dioxane (150 mL) was stirred at 80° C. for 12 hours. Upon completion, the mixture was concentrated under reduced pressure. The crude residue was directly purified by silica gel column chromatography, eluted with petroleum ether/ethyl acetate (5:1), to afford tert-butyl (4-bromo-3-fluorophenyl)carbamate (14.0 g, 92%) as a white solid. LCMS (ESI) [M+H]⁺: 290.0/292.0

### Step 2: tert-butyl (3-fluoro-4-(3,3,3-trifluoroprop-1-en-2-yl)phenyl)carbamate

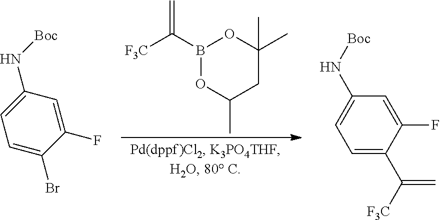 [SMILES: CC(C)(C)OC(=O)Nc1ccc(Br)c(F)c1 ; C=C(B1OC(C)CC(C)(C)O1)C(F)(F)F ; C=C(c1ccc(NC(=O)OC(C)(C)C)cc1F)C(F)(F)F]

A solution of tert-butyl (4-bromo-3-fluorophenyl)carbamate (5.00 g, 17.2 mmol), 4,4,6-trimethyl-2-(3,3,3-trifluoroprop-1-en-2-yl)-1,3,2-dioxaborinane (5.74 g, 25.9 mmol), Pd(dppf)Cl₂ (3.78 g, 5.17 mmol), and tribasic potassium phosphate (11.0 g, 51.7 mmol) in THF (50.0 mL) and water (5.0 mL) was stirred at 80° C. for 16 hours under nitrogen atmosphere. Upon completion, the mixture was cooled to room temperature and concentrated under reduced pressure. The crude residue was directly purified by silica gel column chromatography, eluted with petroleum ether/ethyl acetate (5:1), to afford tert-butyl (3-fluoro-4-(3,3,3-trifluoroprop-1-en-2-yl)phenyl)carbamate (2.90 g, 55% yield) as a pale-yellow solid.

### Step 3: tert-butyl (3-fluoro-4-(1-(trifluoromethyl)cyclopropyl)phenyl)carbamate

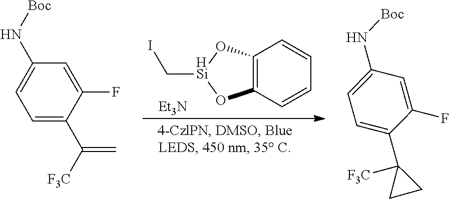

To a mixture of 1,3,2-benzodioxasilole,2-iodomethyl/N,N-diethylethanamine (1.86 g, 4.91 mmol) and 1,2,3,5-tetrakis(carbazol-9-yl)-4,6-dicyanobenzene (4-CZIPN) (129 mg, 0.160 mmol) in DMSO (18.0 mL) at room temperature was added tert-butyl (3-fluoro-4-(3,3,3-trifluoroprop-1-en-2-yl)phenyl)carbamate (1.00 g, 3.27 mmol). The resultant mixture was stirred at room temperature overnight under 450 nm blue LED irradiation. Upon completion, the reaction mixture was poured into ice water and extracted with ethyl acetate (3×20 mL). The combined organic layers were washed with brine (25 mL), dried over anhydrous sodium sulfate, filtered, and concentrated under reduced pressure. The crude residue was purified by silica gel column chromatography, eluted with petroleum ether/ethyl acetate (5:1) to afford tert-butyl (3-fluoro-4-(1-(trifluoromethyl)cyclopropyl)phenyl)carbamate (510 mg, 49%) as a pale-yellow solid. LCMS (ESI) [M+H]⁺: 320.1

### Step 4: 3-fluoro-4-(1-(trifluoromethyl)cyclopropyl)aniline

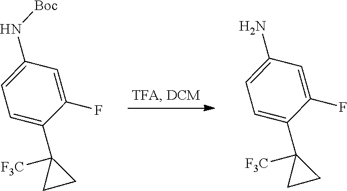 [SMILES: CC(C)(C)OC(=O)Nc1ccc(C2(C(F)(F)F)CC2)c(F)c1 ; Nc1ccc(C2(C(F)(F)F)CC2)c(F)c1]

To a mixture of tert-butyl (3-fluoro-4-(1-(trifluoromethyl)cyclopropyl)phenyl) carbamate (100 mg, 0.310 mmol) in dichloromethane (6.0 mL) at 0° C. was added trifluoroacetic acid (3.0 mL) dropwise. The reaction mixture was warmed to room temperature and stirred for 2 hours. Upon completion, the mixture was concentrated under reduced pressure to afford 3-fluoro-4-(1-(trifluoromethyl)cyclopropyl)aniline (70 mg, crude) as a yellow oil, which was used directly in the next step without further purification. LCMS (ESI) [M+H]⁺: 220.1

### Step 5: tert-butyl (R)-2-((4-bromo-2-hydroxyphenyl)carbamoyl)pyrrolidine-1-carboxylate

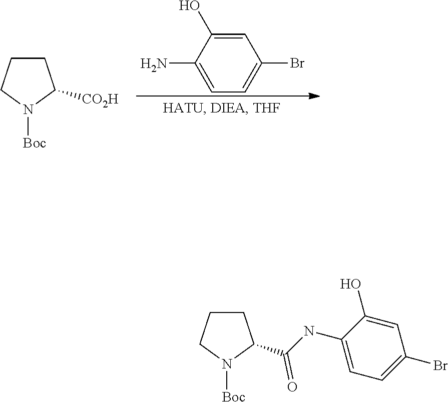

To a mixture of (tert-butoxycarbonyl)-D-proline (1.00 g, 4.64 mmol) in THF (15.0 mL) at 0° C. was added HATU (1.94 g, 5.11 mmol) followed by the dropwise addition of diisopropylethylamine (720 mg, 5.57 mmol). After 30 minutes, 2-amino-5-bromophenol (870 mg, 4.64 mmol) was added dropwise. The reaction mixture was warmed to room temperature and stirred for 12 hours. Upon completion, the mixture was concentrated under reduced pressure. The crude residue was purified by silica gel column chromatography, eluted with petroleum ether/ethyl acetate (1:1) to afford tert-butyl (R)-2-((4-bromo-2-hydroxyphenyl)carbamoyl)pyrrolidine-1-carboxylate (980 mg, 55%) as a brown solid. LCMS (ESI) [M+H]⁺: 385.1

### Step 6: tert-butyl (R)-2-(6-bromobenzo[d]oxazol-2-yl)pyrrolidine-1-carboxylate

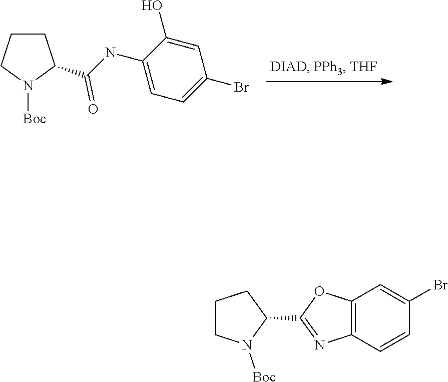

To a mixture of tert-butyl (R)-2-((4-bromo-2-hydroxyphenyl)carbamoyl) pyrrolidine-1-carboxylate (930 mg, 2.41 mmol) and triphenylphosphine (633 mg, 2.41 mmol) in THF (10.0 mL) at 0° C. was added DIAD (488 mg, 2.41 mmol) dropwise. The mixture was warmed to room temperature and stirred for 2 hours. Upon completion, the reaction mixture was cooled to 0° C., quenched with ice water, and extracted with ethyl acetate (3×12 mL). The combined organic layers were dried over anhydrous sodium sulfate, filtered, and concentrated under reduced pressure. The crude residue was purified by silica gel column chromatography, eluted with petroleum ether/ethyl acetate (1:1), to afford tert-butyl (R)-2-(6-bromobenzo[d]oxazol-2-yl)pyrrolidine-1-carboxylate (750 mg, 85% yield) as a light yellow solid. LCMS (ESI) [M+H]⁺: 367.1

### Step 7: tert-butyl (R)-2-(6-(4-(tert-butoxycarbonyl)phenyl)benzo[d]oxazol-2-yl)pyrrolidine-1-carboxylate

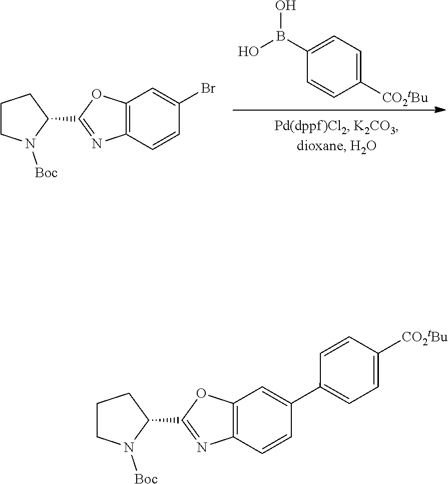 [SMILES: CC(C)(C)OC(=O)N1CCC[C@@H]1c1nc2ccc(Br)cc2o1 ; CC(C)(C)OC(=O)c1ccc(B(O)O)cc1 ; CC(C)(C)OC(=O)c1ccc(-c2ccc3nc([C@H]4CCCN4C(=O)OC(C)(C)C)oc3c2)cc1]

A mixture of tert-butyl (R)-2-(6-bromobenzo[d]oxazol-2-yl)pyrrolidine-1-carboxylate (730 mg, 1.98 mmol), (4-(tert-butoxycarbonyl)phenyl)boronic acid (883 mg, 3.97 mmol), Pd(dppf)Cl₂ (145 mg, 0.190 mmol), and K₃PO₄ (1.27 g, 5.96 mmol) in dioxane (10.0 mL) and water (1.0 mL) was stirred at 80° C. for 2 hours under nitrogen atmosphere. Upon completion, the mixture was cooled to room temperature and poured into ice water. The mixture was extracted with ethyl acetate (3×15 mL). The combined organic layers were washed with brine (18 mL), dried over anhydrous sodium sulfate, filtered, and concentrated under reduced pressure. The crude residue was purified by silica gel column chromatography, eluted with petroleum ether/ethyl acetate (1:1), to afford tert-butyl (R)-2-(6-(4-(tert-butoxycarbonyl)phenyl)benzo[d]oxazol-2-yl)pyrrolidine-1-carboxylate (920 mg, 99%) as a yellow solid. LCMS (ESI) [M+H]⁺: 465.2

### Step 8: (R)-4-(2-(pyrrolidin-2-yl)benzo[d]oxazol-6-yl)benzoic acid

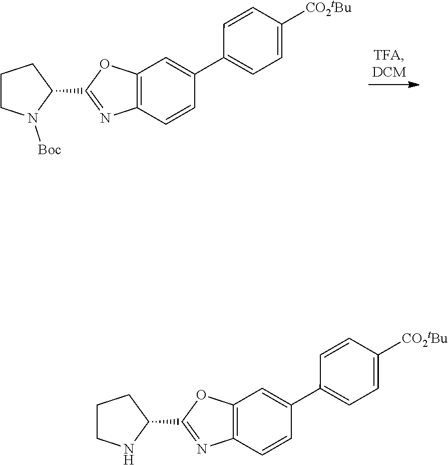 [SMILES: CC(C)(C)OC(=O)c1ccc(-c2ccc3nc([C@H]4CCCN4C(=O)OC(C)(C)C)oc3c2)cc1 ; CC(C)(C)OC(=O)c1ccc(-c2ccc3nc([C@H]4CCCN4)oc3c2)cc1]

To a mixture of tert-butyl (R)-2-(6-(4-(tert-butoxycarbonyl)phenyl)benzo[d]oxazol-2-yl)pyrrolidine-1-carboxylate (910 mg, 1.95 mmol) in dichloromethane (10.0 mL) at 0° C. was added TFA (5.0 mL) dropwise. The mixture was warmed to room temperature and stirred for 12 hours. Upon completion, the mixture was concentrated under reduced pressure to afford (R)-4-(2-(pyrrolidin-2-yl)benzo[d]oxazol-6-yl)benzoic acid (700 mg, crude) as a yellow oil, which was used directly in the next step without further purification. LCMS (ESI) [M+H]⁺: 309.1

### Step 9: (R)-4-(2-(1-((3-fluoro-4-(1-(trifluoromethyl)cyclopropyl)phenyl)carbamoyl)pyrrolidin-2-yl)benzo[d]oxazol-6-yl)benzoic acid

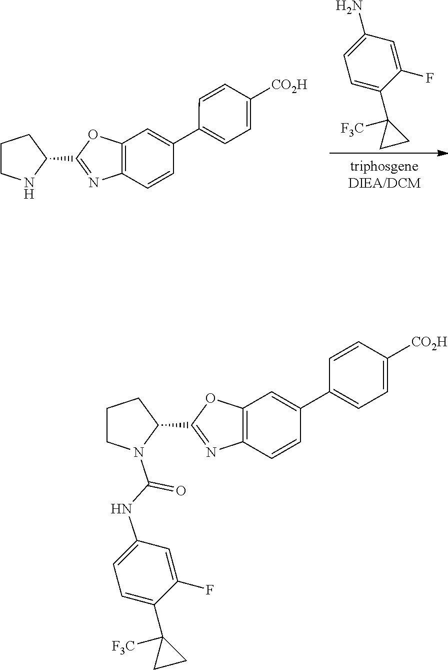 [SMILES: O=C(O)c1ccc(-c2ccc3nc([C@H]4CCCN4)oc3c2)cc1 ; Nc1ccc(C2(C(F)(F)F)CC2)c(F)c1 ; O=C(O)c1ccc(-c2ccc3nc([C@H]4CCCN4C(=O)Nc4ccc(C5(C(F)(F)F)CC5)c(F)c4)oc3c2)cc1]

To a mixture of 3-fluoro-4-(1-(trifluoromethyl)cyclopropyl)aniline (36 mg, 0.160 mmol) and diisopropylethylamine (105 mg, 0.810 mmol) in dichloromethane (5.0 mL) at 0° C. was added triphosgene (19 mg, 65.0 mmol). After 30 minutes, the reaction was warmed to room temperature and (R)-4-(2-(pyrrolidin-2-yl)benzo[d]oxazol-6-yl)benzoic acid (50 mg, 0.160 mmol) was added dropwise over 2 minutes. The reaction was allowed to stir for an additional 2 hours. Upon completion, the mixture was cooled to 0° C. and quenched with ice water. The mixture was extracted with ethyl acetate (3×8 mL). The combined organic layers were dried over anhydrous sodium sulfate, filtered, and concentrated under reduced pressure. The crude residue was purified by preparative HPLC (conditions: column: Xselect CSH Prep C18, 30*150 mm 5 μm; mobile phase A: water (0.1% formic acid), mobile phase B: acetonitrile; flow rate: 60 mL/min; gradient (B %): 47% to 67% over 10 min; detector: 254/220 nm; RT (min): 11.2) to afford (R)-4-(2-(1-((3-fluoro-4-(1-(trifluoromethyl)cyclopropyl)phenyl)carbamoyl) pyrrolidin-2-yl)benzo[d]oxazol-6-yl)benzoic acid (9.3 mg, 11% yield) as a white solid.

| Cmpd | |
|---|---|
| # | Characterization Data |
| 106 | LCMS (ESI) [M + H]⁺: 554.2 |
| | ¹H NMR (400 MHz, DMSO-d₆) δ 12.96 (bs, 1H), |
| | 8.73 (s, 1H), 8.09 (s, 1H), 8.03 (d, J = 8.4 Hz, 2H), 7.86 (d, J = |
| | 8.4 Hz, 2H), 7.80-7.71 (m, 2H), 7.48-7.45 (m, 1H), 7.32-7.30 |
| | (m, 2H), 5.30-5.27 (m, 1H), 3.79-3.78 (m, 1H), 3.63-3.61 (m, |
| | 1H), 2.41-2.38 (m, 1H), 2.15 (bs, 3H), 1.34-1.32 (m, 2H), 1.05 |
| | (s, 2H). |

### Example 109: (S)-4-(5-fluoro-6-(1-((4-isopropylphenyl)carbamoyl)pyrrolidin-2-yl)pyridin-3-yl)-3-methylbenzoic acid & (R)-4-(5-fluoro-6-(1-((4-isopropylphenyl)carbamoyl)pyrrolidin-2-yl)pyridin-3-yl)-3-methylbenzoic acid

### Step 1: methyl 4-(6-chloro-5-fluoropyridin-3-yl)-3-methylbenzoate

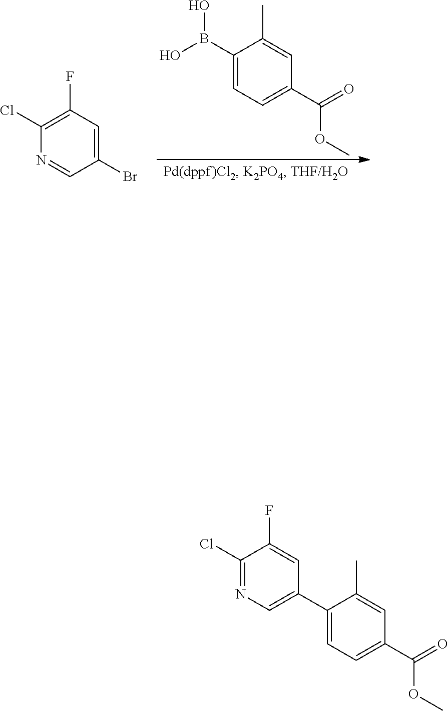 [SMILES: Fc1cc(Br)cnc1Cl ; COC(=O)c1ccc(B(O)O)c(C)c1 ; COC(=O)c1ccc(-c2cnc(Cl)c(F)c2)c(C)c1]

To a stirred solution of 5-bromo-2-chloro-3-fluoropyridine (1.00 g, 4.70 mmol) and 4-(methoxycarbonyl)-2-methylphenylboronic acid (1.11 g, 5.72 mmol) in THF (10.0 mL) under nitrogen atmosphere was added sequentially K₃PO₄ (3.00 g, 14.2 mmol), water (2.0 mL) and Pd(dppf)Cl₂ (0.390 g, 0.500 mmol). The resultant mixture was heated to 80° C. for 2 hours. Upon completion, the reaction mixture was cooled to room temperature and concentrated under reduced pressure. The crude residue was purified by silica gel column chromatography, eluted with petroleum ether/ethyl acetate (5:1), to afford methyl 4-(6-chloro-5-fluoropyridin-3-yl)-3-methylbenzoate (1.00 g, 75%) as a white solid. LCMS (ESI) [M+H]⁺: 280.0

### Step 2: tert-butyl 2-(3-fluoro-5-(4-(methoxycarbonyl)-2-methylphenyl)pyridin-2-yl)pyrrolidine-1-carboxylate

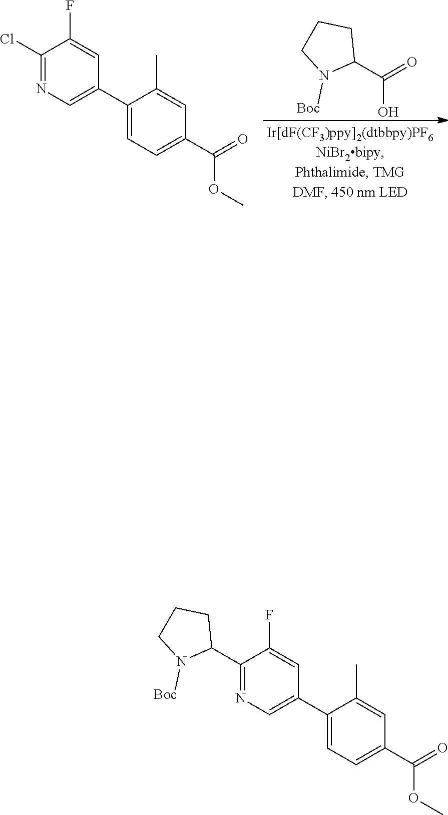 [SMILES: COC(=O)c1ccc(-c2cnc(Cl)c(F)c2)c(C)c1 ; CC(C)(C)OC(=O)N1CCCC1C(=O)O ; COC(=O)c1ccc(-c2cnc(C3CCCN3C(=O)OC(C)(C)C)c(F)c2)c(C)c1]

To a stirred solution of methyl 4-(6-chloro-5-fluoropyridin-3-yl)-3-methylbenzoate (1.00 g, 3.58 mmol) and 1-(tert-butoxycarbonyl)pyrrolidine-2-carboxylic acid (1.15 g, 5.34 mmol) in DMF (10.0 mL) under nitrogen atmosphere was added sequentially [Ir(dF(Me)ppy)₂(dtbbpy)]PF₆ (50 mg, 0.0540 mmol), 2,3-dihydro-1H-isoindole-1,3-dione (0.530 g, 3.58 mmol), N,N,N′,N′-tetramethylguanidine (0.620 g, 5.36 mmol) and [4,4′-Bis(tert-butyl)-2,2′-bipyridine]nickel dibromide (0.170 g, 0.358 mmol), and the resultant mixture was stirred at room temperature under 450 nm blue LED irradiation overnight. Upon completion, the reaction mixture was concentrated under reduced pressure. The crude residue was purified by reversed-phase flash chromatography (conditions: column: C18 silica gel; mobile phase A: water (10 mM formic acid), mobile phase B: acetonitrile; gradient (B %): 10% to 50% over 30 min; detector: 254 nm) to afford tert-butyl 2-(3-fluoro-5-(4-(methoxycarbonyl)-2-methylphenyl)pyridin-2-yl)pyrrolidine-1-carboxylate (500 mg, 34%) as a white solid. LCMS (ESI) [M+H]⁺: 415.2

### Step 3: methyl 4-(5-fluoro-6-(pyrrolidin-2-yl)pyridin-3-yl)-3-methylbenzoate

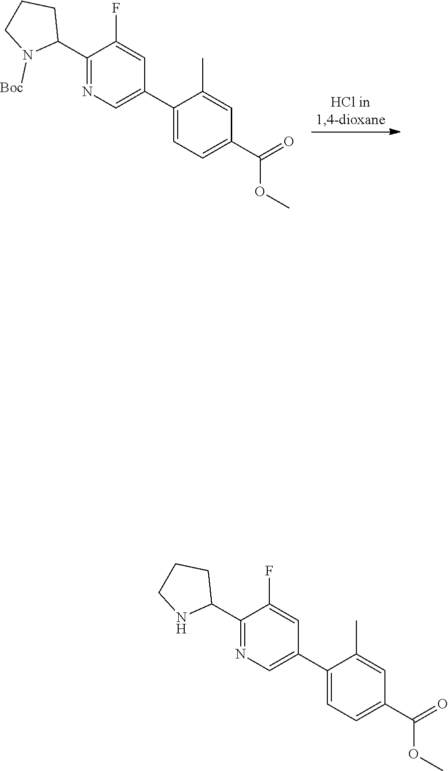 [SMILES: COC(=O)c1ccc(-c2cnc(C3CCCN3C(=O)OC(C)(C)C)c(F)c2)c(C)c1 ; COC(=O)c1ccc(-c2cnc(C3CCCN3)c(F)c2)c(C)c1]

To a solution of tert-butyl 2-(3-fluoro-5-(4-(methoxycarbonyl)-2-methylphenyl)pyridin-2-yl)pyrrolidine-1-carboxylate (500 mg, 1.21 mmol) in dioxane (2.0 mL) at room temperature was added HCl (2.0 mL, 8.0 mmol, 4 M in 1,4-dioxane). The resultant solution was stirred at room temperature for 2 hours. Upon completion, the mixture was concentrated under reduced pressure to afford methyl 4-[5-fluoro-6-(pyrrolidin-2-yl)pyridin-3-yl]-3-methylbenzoate (400 mg, 99%, crude) as a yellow solid, which was used in the next step without further purification. LCMS (ESI) [M+H]⁺: 315.1

### Step 4: methyl 4-(5-fluoro-6-(1-((4-isopropylphenyl)carbamoyl)pyrrolidin-2-yl)pyridin-3-yl)-3-methylbenzoate

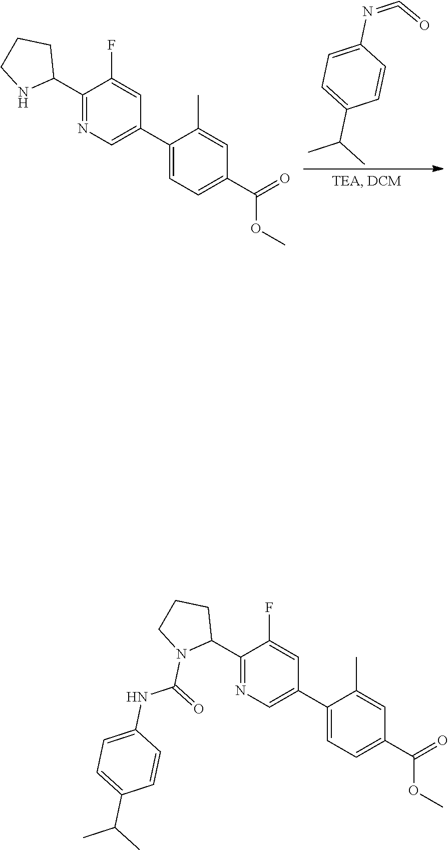

To a stirred solution of methyl 4-[5-fluoro-6-(pyrrolidin-2-yl)pyridin-3-yl]-3-methylbenzoate (300 mg, 0.950 mmol) and 1-isocyanato-4-isopropylbenzene (185 mg, 1.15 mmol) in DCM (5.0 mL) was added triethylamine (290 mg, 2.86 mmol) at room temperature. The resultant mixture was stirred at room temperature for 2 hours. Upon completion, the mixture was concentrated under reduced pressure. The crude residue was purified by silica gel column chromatography, eluted with petroleum ether/ethyl acetate (5:1), to afford methyl 4-(5-fluoro-6-(1-((4-isopropylphenyl)carbamoyl)pyrrolidin-2-yl)pyridin-3-yl)-3-methylbenzoate (200 mg, 44%) as a yellow solid. LCMS (ESI) [M+H]⁺: 476.2.

### Step 5: 4-(5-fluoro-6-(1-((4-isopropylphenyl)carbamoyl)pyrrolidin-2-yl)pyridin-3-yl)-3-methylbenzoic acid

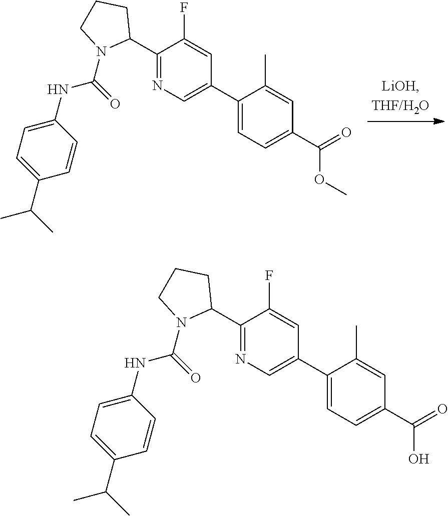

To a stirred solution of methyl 4-(5-fluoro-6-(1-((4-isopropylphenyl)carbamoyl)pyrrolidin-2-yl)pyridin-3-yl)-3-methylbenzoate (200 mg, 0.421 mmol) and LiOH monohydrate (84.0 mg, 2.00 mmol) in THF (2.0 mL) was added water (0.50 mL). The resultant mixture was heated to 50° C. for 2 hours. Upon completion, the mixture was cooled to room temperature and concentrated under reduced pressure. The crude residue was purified by reversed-phase flash chromatography (conditions: column: C18 silica gel; mobile phase A: water (10 mM formic acid); mobile phase B: acetonitrile; gradient (B %): 10% to 70% over 24 min; detector: 254 nm) to afford 4-(5-fluoro-6-(1-((4-isopropylphenyl)carbamoyl)pyrrolidin-2-yl)pyridin-3-yl)-3-methylbenzoic acid (150 mg, 77%) as a yellow solid. LCMS (ESI) [M+H]⁺: 462.2

### Step 6: (S)-4-(5-fluoro-6-(1-((4-isopropylphenyl)carbamoyl)pyrrolidin-2-yl)pyridin-3-yl)-3-methylbenzoic acid & (R)-4-(5-fluoro-6-(1-((4-isopropylphenyl)carbamoyl)pyrrolidin-2-yl)pyridin-3-yl)-3-methylbenzoic acid

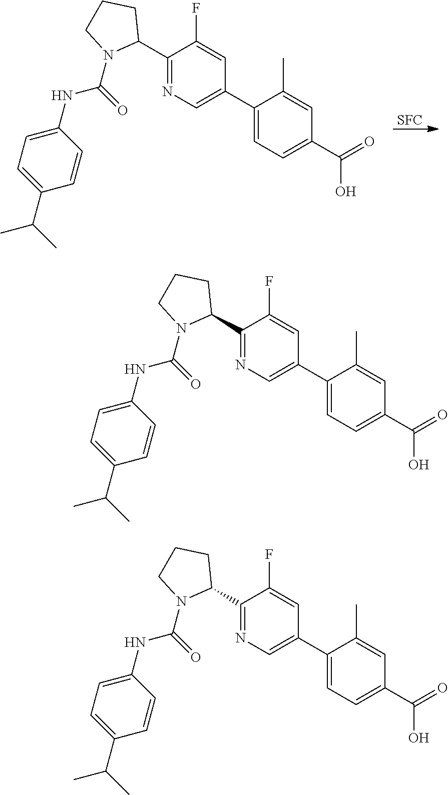

4-(5-Fluoro-6-(1-((4-isopropylphenyl)carbamoyl)pyrrolidin-2-yl)pyridin-3-yl)-3-methylbenzoic acid (100 mg, 0.217 mmol) was resolved by preparative-SFC (conditions: column: CHIRAL ART Cellulose-SC, 3*25 cm, 5 μm; mobile phase A: hexane (0.1% formic acid); mobile phase B: ethyl acetate; Flow rate: 40 mL/min; gradient (B %): isocratic 30% B; detector: 220/254 nm; RT1 (min): 6.4; RT2 (min): 8.2) to afford (Compound 109a; Method A, Peak 1, 0.84 min) (S)-4-(5-fluoro-6-(1-((4-isopropylphenyl)carbamoyl)pyrrolidin-2-yl)pyridin-3-yl)-3-methylbenzoic acid (1^{st} peak, 32.6 mg, 32%) as a white solid and (Compound 109b; Method A, Peak 2, 1.15 min) (R)-4-(5-fluoro-6-(1-((4-isopropylphenyl)carbamoyl)pyrrolidin-2-yl)pyridin-3-yl)-3-methylbenzoic acid (2^{nd} peak, 28.0 mg, 28%) as a white solid.

| Cmpd # | Characterization Data |
|---|---|
| 109a | LCMS (ESI) [M + H]⁺: 462.2 |
| | ¹H NMR (400 MHz, DMSO-d6) δ 13.01 (bs, 1H), 8.37 (s, 1H), 8.14 (s, 1H), 7.90 (d, J = 1.9 Hz, 1H), |
| | 7.86-7.74 (m, 2H), 7.39 (d, J = 8.0 Hz, 1H), 7.34 (d, J = 8.5 Hz, 2H), 7.05 (d, J = 8.5 Hz, 2H), 5.36 |
| | (dd, J = 8.3, 4.0 Hz, 1H), 3.82-3.70 (m, 1H), 3.68-3.55 (m, 1H), 2.78 (hept, J = 6.9 Hz, 1H), 2.41- |
| | 2.24 (m, 4H), 2.18-2.06 (m, 1H), 2.06-1.95 (m, 1H), 1.95-1.86 (m, 1H), 1.15 (d, J = 6.9 Hz, 6H). |
| 109b | LCMS (ESI) [M + H]⁺: 462.2 |
| | ¹H NMR (400 MHz, DMSO-d6) δ 12.99 (bs, 1H), 8.37 (s, 1H), 8.14 (s, 1H), 7.90 (d, J = 1.7 Hz, 1H), |
| | 7.84-7.75 (m, 2H), 7.40 (d, J = 8.0 Hz, 1H), 7.34 (d, J = 8.6 Hz, 2H), 7.05 (d, J = 8.6 Hz, 2H), 5.36 |
| | (dd, J = 8.4, 4.0 Hz, 1H), 3.83-3.69 (m, 1H), 3.66-3.55 (m, 1H), 2.78 (hept, J = 6.9 Hz, 1H), 2.43- |
| | 2.27 (m, 4H), 2.17-2.07 (m, 1H), 2.07-1.95 (m, 1H), 1.95-1.85 (m, 1H), 1.15 (d, J = 6.9 Hz, 6H). |

### Example 110: (S)-4-(5-fluoro-6-(1-((4-isopropylphenyl)carbamoyl)pyrrolidin-2-yl)-2-methylpyridin-3-yl)benzoic acid & (R)-4-(5-fluoro-6-(1-((4-isopropylphenyl)carbamoyl)pyrrolidin-2-yl)-2-methylpyridin-3-yl)benzoic acid

### Step 1: 5-bromo-3-fluoro-6-methylpyridin-2-amine

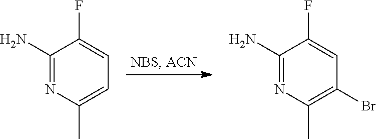

To a stirred solution of 3-fluoro-6-methylpyridin-2-amine (2.00 g, 15.8 mmol) in acetonitrile (20 mL) was added NBS (3.10 g, 17.4 mmol) at 0° C., and the resultant mixture was warmed to room temperature and stirred for 1 hour. Upon completion, the mixture was concentrated under reduced pressure. The crude residue was purified by silica gel column chromatography, eluted with petroleum ether/ethyl acetate (5:1), to afford 5-bromo-3-fluoro-6-methylpyridin-2-amine (2.00 g, 61%) as a yellow solid. LCMS (ESI) [M+H]⁺: 205.0

### Step 2: methyl 4-(6-amino-5-fluoro-2-methylpyridin-3-yl)benzoate

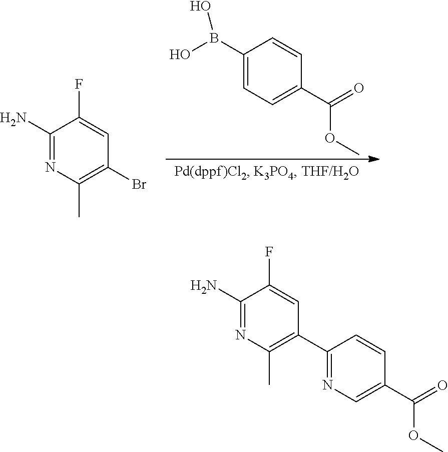

To a stirred solution of 5-bromo-3-fluoro-6-methylpyridin-2-amine (1.00 g, 4.88 mmol) and 4-(methoxycarbonyl)phenylboronic acid (1.05 g, 5.83 mmol) in THF (10.0 mL) was added sequentially Pd(dppf)Cl₂ (398.3 mg, 0.490 mmol), water (2.0 mL), and K₃PO₄ (3.20 g, 14.6 mmol), and the resultant mixture was heated to 80° C. for 2 hours. Upon completion, the mixture was filtered. The filter cake was washed with ethyl acetate (3×10 mL). The filtrate was concentrated under reduced pressure. The crude residue was purified by silica gel column chromatography, eluting with petroleum ether/ethyl acetate (5:1), to afford methyl 4-(6-amino-5-fluoro-2-methylpyridin-3-yl)benzoate (800 mg, 63%) as a yellow solid. LCMS (ESI) [M+H]⁺: 261.2

### Step 3: methyl 4-(6-bromo-5-fluoro-2-methylpyridin-3-yl)benzoate

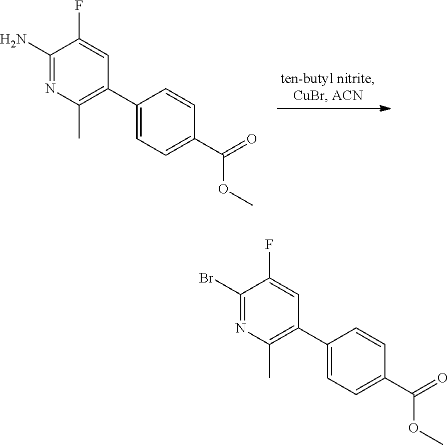

To a stirred solution of methyl 4-(6-amino-5-fluoro-2-methylpyridin-3-yl)benzoate (800 mg, 3.07 mmol) and CuBr (2.20 g, 15.4 mmol) in acetonitrile (10.0 mL) was added tert-butylnitrite (1.60 g, 15.4 mmol), and the resultant mixture was heated to 60° C. for 2 hours. Upon completion, the mixture was cooled to room temperature, filtered, and the filter cake was washed with acetonitrile (3×30 mL). The filtrate was concentrated under reduced pressure. The crude residue was purified by silica gel column chromatography, eluted with petroleum ether/ethyl acetate (5:1), to afford methyl 4-(6-bromo-5-fluoro-2-methylpyridin-3-yl)benzoate (600 mg, 60%) as a yellow solid. LCMS (ESI) [M+H]⁺: 324.1

### Step 4: tert-butyl 2-(3-fluoro-5-(4-(methoxycarbonyl)phenyl)-6-methylpyridin-2-yl)pyrrolidine-1-carboxylate

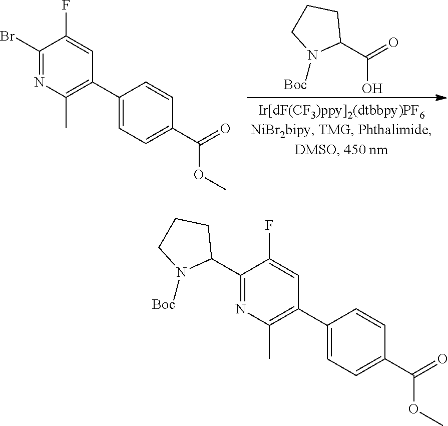

To a stirred solution of methyl 4-(6-bromo-5-fluoro-2-methylpyridin-3-yl)benzoate (600 mg, 1.85 mmol) and 1-(tert-butoxycarbonyl)pyrrolidine-2-carboxylic acid (600 mg, 2.80 mmol) in DMSO (10.0 mL) at room temperature were added [Ir(dF(Me)ppy)₂(dtbbpy)]PF₆ (28.2 mg, 0.0278 mmol), 2,3-dihydro-1H-isoindole-1,3-dione (272 mg, 1.90 mmol), N,N,N′,N′-tetramethylguanidine (320 mg, 2.80 mmol), and [4,4′-Bis(tert-butyl)-2,2′-bipyridine]nickel dibromide (90.0 mg, 0.184 mmol). The resultant mixture was then stirred at room temperature under 450 nM blue light LED irradiation overnight. Upon completion, the mixture was filtered, and the filter cake was washed with methanol (3×20 mL). The filtrate was concentrated under reduced pressure. The crude residue was purified by reversed-phase flash chromatography (conditions: column: C18 silica gel; mobile phase A: water (10 mM formic acid); mobile phase B: acetonitrile; gradient (B %): 10% to 80% over 20 min; detector: 254 nm) to afford methyl 4-(5-fluoro-6-(1-((4-isopropylphenyl)carbamoyl)pyrrolidin-2-yl)pyridin-3-yl)-3-methylbenzoate (300 mg, 39%) as a yellow solid. LCMS (ESI) [M+H]⁺: 415.2

### Step 5: methyl 4-(5-fluoro-2-methyl-6-(pyrrolidin-2-yl)pyridin-3-yl)benzoate

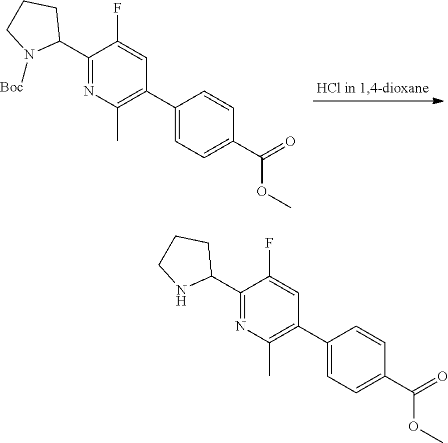

To a stirred solution of methyl 4-(5-fluoro-6-(1-((4-isopropylphenyl)carbamoyl)pyrrolidin-2-yl)pyridin-3-yl)-3-methylbenzoate (300 mg, 0.724 mmol) in dioxane (3.0 mL) was added HCl (3.0 mL, 12.0 mmol, 4.0 M in dioxane) at room temperature, and the resultant mixture was stirred for 2 hours. Upon completion, the mixture was concentrated under reduced pressure to afford methyl 4-(5-fluoro-2-methyl-6-(pyrrolidin-2-yl)pyridin-3-yl)benzoate (200 mg, crude) as a yellow solid, which was used in the next step without further purification. LCMS (ESI) [M+H]⁺: 315.1

### Step 6: methyl 4-(5-fluoro-6-(1-((4-isopropylphenyl)carbamoyl)pyrrolidin-2-yl)-2-methylpyridin-3-yl)benzoate

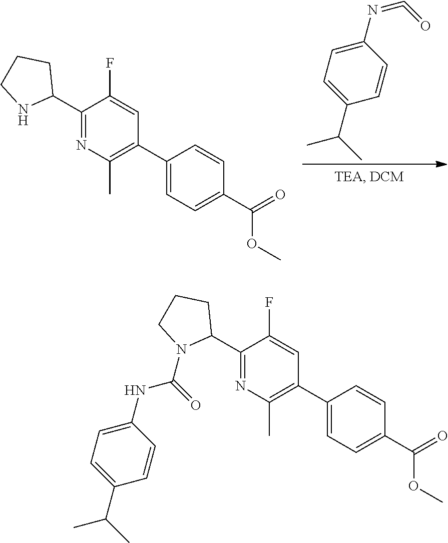

To a stirred solution of methyl 4-[5-fluoro-2-methyl-6-(pyrrolidin-2-yl)pyridin-3-yl]benzoate (200 mg, 0.636 mmol) and 1-isocyanato-4-isopropylbenzene (123 mg, 0.763 mmol) in DCM (3.0 mL) was added triethylamine (193 mg, 1.90 mmol) at room temperature, and the resultant mixture was stirred for 2 hours. Upon completion, the mixture was concentrated under reduced pressure. The crude residue was directly purified by silica gel column chromatography, eluted with petroleum ether/ethyl acetate (5:1), to afford methyl 4-(5-fluoro-6-(1-((4-isopropylphenyl)carbamoyl)pyrrolidin-2-yl)-2-methylpyridin-3-yl)benzoate (200 mg, 66%) as a yellow solid. LCMS (ESI) [M+H]⁺: 476.2

### Step 7: 4-(5-fluoro-6-(1-((4-isopropylphenyl)carbamoyl)pyrrolidin-2-yl)-2-methylpyridin-3-yl)benzoic acid

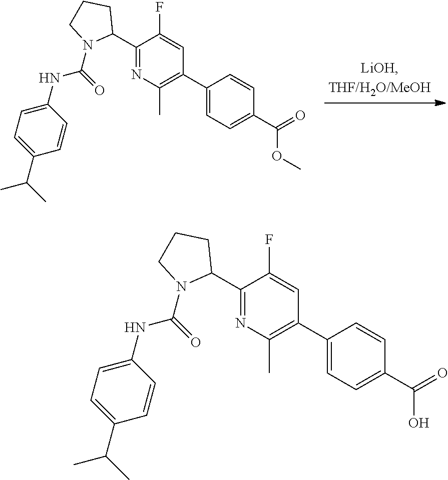 [SMILES: COC(=O)c1ccc(-c2cc(F)c(C3CCCN3C(=O)Nc3ccc(C(C)C)cc3)nc2C)cc1 ; Cc1nc(C2CCCN2C(=O)Nc2ccc(C(C)C)cc2)c(F)cc1-c1ccc(C(=O)O)cc1]

To a stirred solution of methyl 4-(5-fluoro-6-(1-((4-isopropylphenyl)carbamoyl)pyrrolidin-2-yl)-2-methylpyridin-3-yl)benzoate (100 mg, 0.210 mmol) and LiOH monohydrate (42.0 mg, 1.00 mmol) in THF (1.0 mL) and methanol (1.0 mL) was added water (0.20 mL), and the resultant mixture was heated to 50° C. for 1 hour. Upon completion, the mixture was concentrated under reduced pressure. The crude residue was purified by reversed-phase flash chromatography (conditions: column: C18 silica gel; mobile phase A: water (10 mM formic acid), mobile phase B: acetonitrile; gradient (B %): 10% to 95% over 26 min; detector: 254 nm) to afford 4-(5-fluoro-6-(1-((4-isopropylphenyl)carbamoyl)pyrrolidin-2-yl)-2-methylpyridin-3-yl)benzoic acid (80.0 mg, 82%) as a yellow solid. LCMS (ESI) [M+H]⁺: 462.2

### Step 8: (S)-4-(5-fluoro-6-(1-((4-isopropylphenyl)carbamoyl)pyrrolidin-2-yl)-2-methylpyridin-3-yl)benzoic acid & (R)-4-(5-fluoro-6-(1-((4-isopropylphenyl)carbamoyl)pyrrolidin-2-yl)-2-methylpyridin-3-yl)benzoic acid

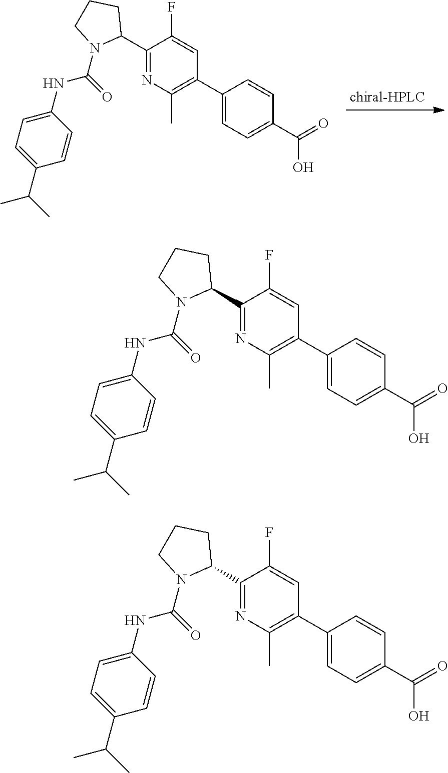 [SMILES: Cc1nc(C2CCCN2C(=O)Nc2ccc(C(C)C)cc2)c(F)cc1-c1ccc(C(=O)O)cc1 ; Cc1nc([C@@H]2CCCN2C(=O)Nc2ccc(C(C)C)cc2)c(F)cc1-c1ccc(C(=O)O)cc1 ; Cc1nc([C@H]2CCCN2C(=O)Nc2ccc(C(C)C)cc2)c(F)cc1-c1ccc(C(=O)O)cc1]

4-(5-Fluoro-6-(1-((4-isopropylphenyl)carbamoyl)pyrrolidin-2-yl)-2-methylpyridin-3-yl)benzoic acid (80.0 mg) was resolved by prep Chiral HPLC (conditions: Column: Enantiocel-A4-5; Mobile Phase A: Hexane (0.1% FA), Mobile Phase B: EtOH; Flow rate: 40 mL/min; Gradient (B %): isocratic 50% B; Wave Length: 220/254 nm; RT1 (min): 8.2; RT2 (min): 16.2) to afford (Compound 110a; Method AJ, Peak 1, 1.29 min) (S)-4-(5-fluoro-6-(1-((4-isopropylphenyl)carbamoyl)pyrrolidin-2-yl)-2-methylpyridin-3-yl)benzoic acid (1^{st} peak, 20.0 mg, 25%) as a white solid, and (Compound 110b; Method AJ, Peak 1, 2.59 min) (R)-4-(5-fluoro-6-(1-((4-isopropylphenyl)carbamoyl)pyrrolidin-2-yl)-2-methylpyridin-3-yl)benzoic acid (2^{nd} peak, 24.4 mg, 31%) as a white solid.

| Cmpd # | Characterization Data |
|---|---|
| 110a | LCMS (ESI) [M + H]⁺: 462.1 |
| | ¹H NMR (400 MHz, DMSO-d6) δ 13.09 (bs, 1H), 8.14 (s, 1H), 8.01 (d, J = 8.3 Hz, 2H), 7.55 (dd, |
| | J = 9.5, 2.4 Hz, 3H), 7.35 (d, J = 8.6 Hz, 2H), 7.05 (d, J = 8.6 Hz, 2H), 5.29 (dd, J = 8.0, 4.4 Hz, 1H), |
| | 3.79-3.67 (m, 1H), 3.65-3.54 (m, 1H), 2.78 (hept, J = 6.9 Hz, 1H), 2.38-2.28 (m, 4H), 2.21- |
| | 2.13 (m, 1H), 2.06-1.94 (m, 1H), 1.94-1.83 (m, 1H), 1.15 (d, J = 6.9 Hz, 6H). |
| 110b | LCMS (ESI) [M + H]⁺: 462.2 |
| | ¹H NMR (400 MHz, DMSO-d6) δ 13.17 (bs, 1H), 8.16 (s, 1H), 8.00 (d, J = 8.3 Hz, 2H), 7.54 (dd, |
| | J = 9.4, 6.6 Hz, 3H), 7.35 (d, J = 8.6 Hz, 2H), 7.05 (d, J = 8.6 Hz, 2H), 5.29 (dd, J = 8.1, 4.3 Hz, 1H), |
| | 3.78-3.69 (m, 1H), 3.64-3.55 (m, 1H), 2.78 (hept, J = 6.9 Hz, 1H), 2.39-2.26 (m, 4H), 2.22- |
| | 2.08 (m, 1H), 2.04-1.96 (m, 1H), 1.96-1.84 (m, 1H), 1.15 (d, J = 6.9 Hz, 6H). |

### Example 115: (S)-4-(5-fluoro-6-(1-((4-isopropylbenzyl)carbamoyl)pyrrolidin-2-yl)pyridin-3-yl)benzoic acid and (R)-4-(5-fluoro-6-(1-((4-isopropylbenzyl)carbamoyl)pyrrolidin-2-yl)pyridin-3-yl)benzoic acid

### Step 1: 4-(5-fluoro-6-(1-((4-isopropylbenzyl)carbamoyl)pyrrolidin-2-yl)pyridin-3-yl)benzoic acid

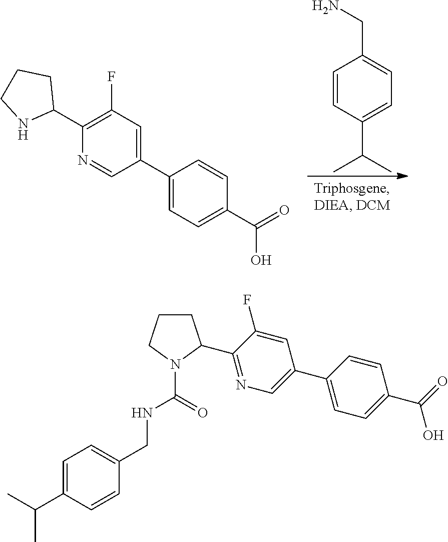

To a mixture of (4-isopropylphenyl)methanamine (52.0 mg, 0.349 mmol) and DIEA (226 mg, 1.80 mmol) in DCM (5.0 mL) was added triphosgene (42.0 mg, 0.141 mmol) dropwise at 0° C. After 30 minutes, the reaction was warmed to room temperature and 4-(5-fluoro-6-(pyrrolidin-2-yl)pyridin-3-yl)benzoic acid (100 mg, 0.350 mmol) was added dropwise over 3 minutes. The mixture was stirred for an additional 2 hours. Upon completion, the reaction was cooled 0° C. and quenched with ice water. The mixture was extracted with ethyl acetate (3×15 mL). The combined organic layers were dried over anhydrous Na₂SO₄, filtered, and concentrated under reduced pressure. The crude residue was purified by reversed-phase flash chromatography (conditions: column: C18 silica gel; mobile phase A: water (0.1% formic acid), mobile phase B: acetonitrile; gradient (B %): 0% to 100% over 25 min; detector: 254 nm) to afford 4-(5-fluoro-6-(1-((4-isopropylbenzyl)carbamoyl)pyrrolidin-2-yl)pyridin-3-yl)benzoic acid (100 mg, 62%) as an off-white solid. LCMS [M+H]⁺: 462.0

### Step 2: (S)-4-(5-fluoro-6-(1-((4-isopropylbenzyl)carbamoyl)pyrrolidin-2-yl)pyridin-3-yl)benzoic acid and (R)-4-(5-fluoro-6-(1-((4-isopropylbenzyl)carbamoyl)pyrrolidin-2-yl)pyridin-3-yl)benzoic acid

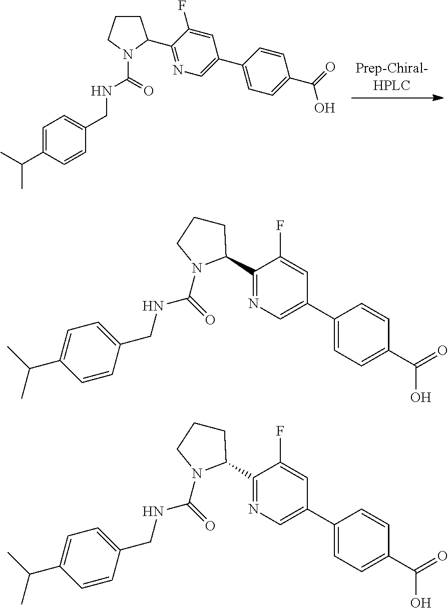 [SMILES: CC(C)c1ccc(CNC(=O)N2CCCC2c2ncc(-c3ccc(C(=O)O)cc3)cc2F)cc1 ; CC(C)c1ccc(CNC(=O)N2CCC[C@H]2c2ncc(-c3ccc(C(=O)O)cc3)cc2F)cc1 ; CC(C)c1ccc(CNC(=O)N2CCC[C@@H]2c2ncc(-c3ccc(C(=O)O)cc3)cc2F)cc1]

4-(5-fluoro-6-(1-((4-isopropylbenzyl)carbamoyl)pyrrolidin-2-yl)pyridin-3-yl)benzoic acid (100 mg) was resolved by preparative chiral HPLC (conditions: column: CHIRAL ART Cellulose-SB, 3*25 cm, 5 μm; mobile phase A: hexane (0.1% formic acid), mobile phase B: ethanol; Flow rate: 40 mL/min; gradient (B %): isocratic 50% B; Detector: 220/254 nm; RT1 (min): 7.2; RT2 (min): 10.2) to afford first peak (Compound 115a; Method M, Peak 1, 0.70 min) (S)-4-(5-fluoro-6-(1-((4-isopropylbenzyl)carbamoyl)pyrrolidin-2-yl)pyridin-3-yl)benzoic acid (38.4 mg, 38%) as a white solid and second peak (Compound 115b; Method M, Peak 2, 1.03 min) (R)-4-(5-fluoro-6-(1-((4-isopropylbenzyl)carbamoyl)pyrrolidin-2-yl)pyridin-3-yl)benzoic acid (38.9 mg, 39%) as a white solid.

| Cmpd # | Characterization Data |
|---|---|
| 115a | LCMS (ESI) [M + H]⁺: 462.2 |
| | ¹H NMR (400 MHz, DMSO-d₆) δ 13.04 (bs, 1H), 8.75 (s, 1H), 8.05-8.03 (m, 3H), 7.90 (d, J = 8.4 |
| | Hz, 2H), 7.12-7.08 (m, 4H), 6.70-6.67 (m, 1H), 5.25-5.22 (m, 1H), 4.20-4.14 (m, 1H), 4.09- |
| | 4.03 (m, 1H), 3.62-3.58 (m, 1H), 3.50-3.46 (m, 1H), 2.83-2.67 (m, 1H), 2.33-2.28 (m, 1H), 2.07- |
| | 2.05 (m, 1H), 1.97-1.94 (m, 1H), 1.87-1.83 (m, 1H), 1.15 (d, J = 8.4 Hz, 6H). |
| 115b | LCMS (ESI) [M + H]⁺: 462.2 |
| | ¹H NMR (400 MHz, DMSO-d₆) δ 13.09 (bs, 1H), 8.75 (s, 1H), 8.06-8.03 (m, 3H), 7.90 (d, J = 8.4 |
| | Hz, 2H), 7.13-7.08 (m, 4H), 6.71-6.68 (m, 1H), 5.25-5.22 (m, 1H), 4.20-4.14 (m, 1H), 4.09- |
| | 4.03 (m, 1H), 3.62-3.58 (m, 1H), 3.50-3.46 (m, 1H), 2.83-2.80 (m, 1H), 2.33-2.28 (m, 1H), 2.06- |
| | 2.03 (m, 1H), 1.97-1.94 (m, 1H), 1.87-1.84 (m, 1H), 1.15 (d, J = 6.8 Hz, 6H). |

### Example 116: (S)-2-(4-(6-((S)-1-((4-isopropylphenyl)carbamoyl)pyrrolidin-2-yl)pyridin-3-yl)phenyl)propanoic acid, (R)-2-(4-(6-((S)-1-((4-isopropylphenyl)carbamoyl)pyrrolidin-2-yl)pyridin-3-yl)phenyl)propanoic acid, (S)-2-(4-(6-((R)-1-((4-isopropylphenyl)carbamoyl)pyrrolidin-2-yl)pyridin-3-yl)phenyl)propanoic acid and (R)-2-(4-(6-((R)-1-((4-isopropylphenyl)carbamoyl)pyrrolidin-2-yl)pyridin-3-yl)phenyl)propanoic acid

### Step 1: 2-(5-bromopyridin-2-yl)-N-(4-isopropylphenyl)pyrrolidine-1-carboxamide

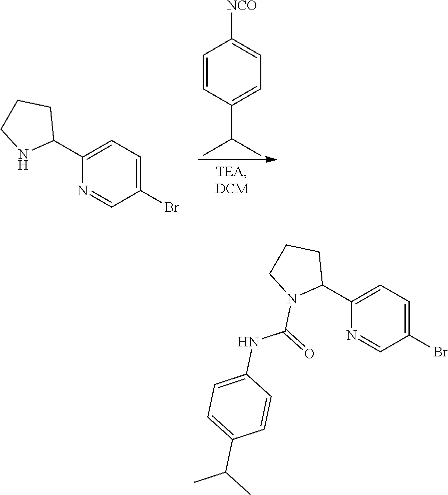

To a mixture of 5-bromo-2-(pyrrolidin-2-yl)pyridine (1.50 g, 6.60 mmol) in DCM (20.0 mL) were added triethylamine (1.00 g, 9.90 mmol) and 1-isocyanato-4-isopropylbenzene (1.28 g, 7.90 mmol) at 0° C. The mixture was warmed to room temperature and stirred for 1 hour. Upon completion, the reaction mixture was cooled to 0° C. and quenched with ice water. The aqueous layer was extracted with ethyl acetate (3×60 mL). The combined organic layers were dried over anhydrous Na₂SO₄, filtered, and concentrated under reduced pressure. The crude residue was purified by silica gel column chromatography, eluted with petroleum ether/ethyl acetate (1:1), to afford 2-(5-bromopyridin-2-yl)-N-(4-isopropylphenyl)pyrrolidine-1-carboxamide (1.20 g, 47%) as a white solid. LCMS [M+H]⁺: 388

### Step 2: methyl 2-(4-(6-(1-((4-isopropylphenyl)carbamoyl)pyrrolidin-2-yl)pyridin-3-yl)phenyl)propanoate

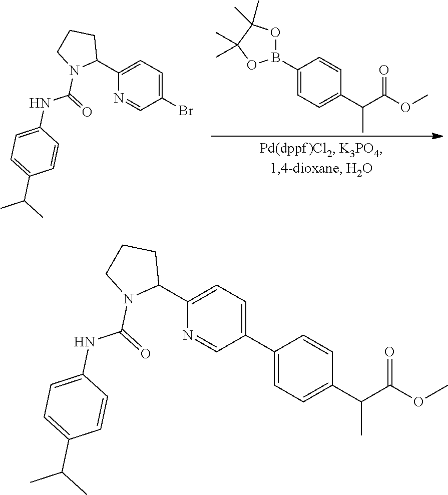

To a mixture of 2-(5-bromopyridin-2-yl)-N-(4-isopropylphenyl)pyrrolidine-1-carboxamide (200 mg, 0.517 mmol) in 1,4-dioxane (4.0 mL) were added water (1.0 mL), K₃PO₄ (219 mg, 1.00 mmol), methyl 2-(4-(4,4,5,5-tetramethyl-1,3,2-dioxaborolan-2-yl)phenyl)propanoate (224 mg, 0.841 mmol) and Pd(dppf)Cl₂ (38.0 mg, 52.0 μmol) at room temperature. The reaction mixture was then heated to 80° C. and stirred for 1 hour. Upon completion, the mixture was cooled to room temperature and filtered. The filtrate was concentrated under reduced pressure. The crude residue was purified by silica gel column chromatography, eluted with petroleum ether/ethyl acetate (1:1), to afford methyl 2-(4-(6-(1-((4-isopropylphenyl)carbamoyl)pyrrolidin-2-yl)pyridin-3-yl)phenyl)propanoate (230 mg, 95%) as a white solid. LCMS [M+H]⁺: 472

### Step 3: 2-(4-(6-(1-((4-isopropylphenyl)carbamoyl)pyrrolidin-2-yl)pyridin-3-yl)phenyl)propanoic acid

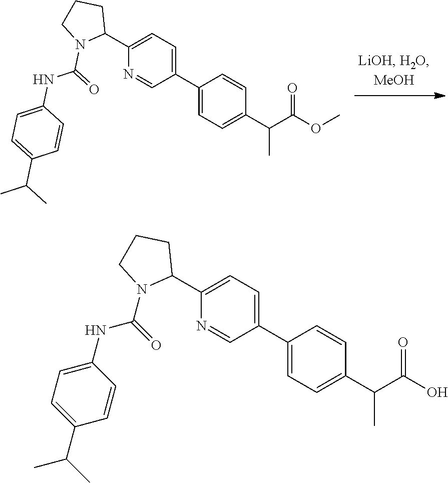

To a mixture of methyl 2-(4-(6-(1-((4-isopropylphenyl)carbamoyl)pyrrolidin-2-yl)pyridin-3-yl)phenyl)propanoate (220 mg, 0.467 mmol) in methanol (3.0 mL) were added water (3.0 mL) and LiOH monohydrate (38 mg, 0.905 mmol) at room temperature, and the resultant mixture was stirred for 12 hours. Upon completion, the mixture was acidified to pH ˜3 with HCl (1 M). The resultant mixture was extracted with ethyl acetate (3×50 mL). The combined organic layers were dried over anhydrous Na₂SO₄, filtered, and concentrated under reduced pressure. The crude residue was purified by reversed-phase flash chromatography (conditions: column: C18 silica gel; mobile phase A: water (0.1% formic acid), mobile phase B: acetonitrile; gradient (B %): 0% to 100% over 25 min; detector: 254 nm) to afford 2-(4-(6-(1-((4-isopropylphenyl)carbamoyl)pyrrolidin-2-yl)pyridin-3-yl)phenyl)propanoic acid (130 mg, 61%) as a white solid. LCMS [M+H]⁺: 458

### Step 4: (S)-2-(4-(6-((S)-1-((4-isopropylphenyl)carbamoyl)pyrrolidin-2-yl)pyridin-3-yl)phenyl)propanoic acid, (R)-2-(4-(6-((S)-1-((4-isopropylphenyl)carbamoyl)pyrrolidin-2-yl)pyridin-3-yl)phenyl)propanoic acid, (S)-2-(4-(6-((R)-1-((4-isopropylphenyl)carbamoyl)pyrrolidin-2-yl)pyridin-3-yl)phenyl)propanoic acid and (R)-2-(4-(6-((R)-1-((4-isopropylphenyl)carbamoyl)pyrrolidin-2-yl)pyridin-3-yl)phenyl)propanoic

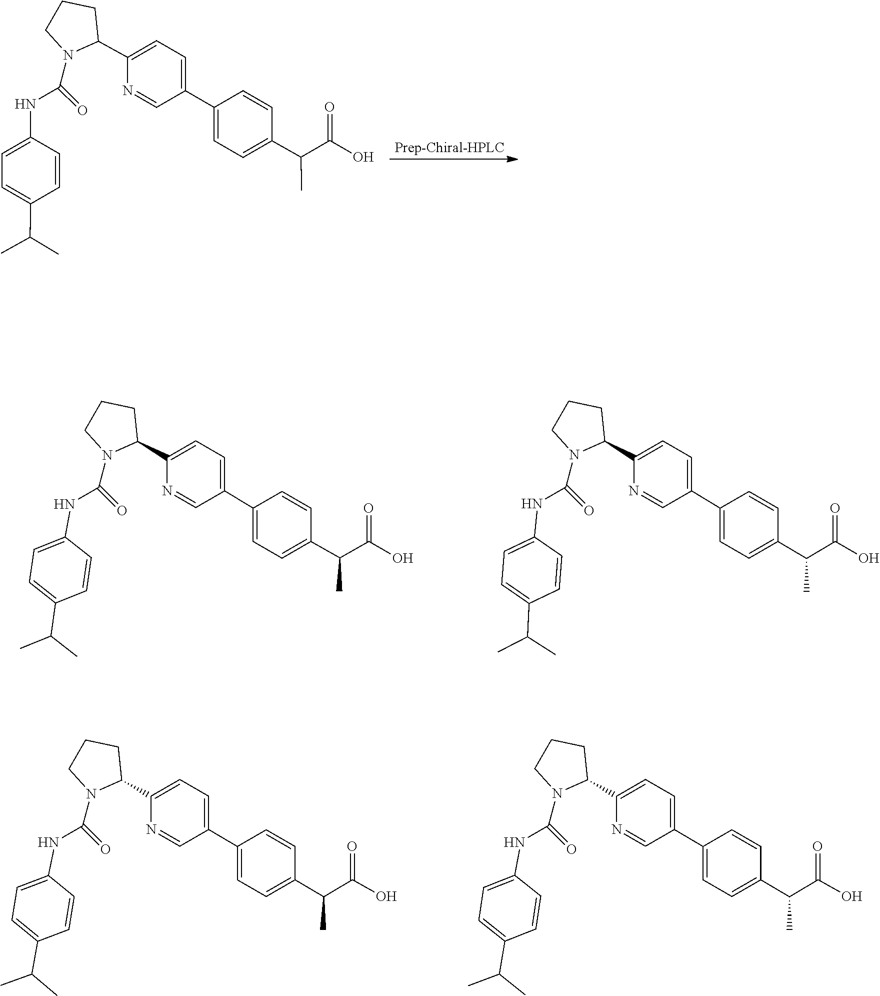 [SMILES: CC(C)c1ccc(NC(=O)N2CCCC2c2ccc(-c3ccc(C(C)C(=O)O)cc3)cn2)cc1 ; CC(C)c1ccc(NC(=O)N2CCC[C@H]2c2ccc(-c3ccc([C@H](C)C(=O)O)cc3)cn2)cc1 ; CC(C)c1ccc(NC(=O)N2CCC[C@H]2c2ccc(-c3ccc([C@@H](C)C(=O)O)cc3)cn2)cc1 ; CC(C)c1ccc(NC(=O)N2CCC[C@@H]2c2ccc(-c3ccc([C@H](C)C(=O)O)cc3)cn2)cc1 ; CC(C)c1ccc(NC(=O)N2CCC[C@@H]2c2ccc(-c3ccc([C@@H](C)C(=O)O)cc3)cn2)cc1]

2-(4-(6-(1-((4-isopropylphenyl)carbamoyl)pyrrolidin-2-yl)pyridin-3-yl)phenyl)propanoic acid (125 mg) was resolved by preparative chiral HPLC (conditions: column: CHIRALPAK ID, 3*25 cm, 5 μm; mobile phase A: hexane (0.1% formic acid), mobile phase B: ethanol; Flow rate: 40 mL/min; gradient (B %): isocratic 50% B; detector: 220/254 nm; RT1 (min): 10.2; RT2 (min): 14.3; RT3 (min): 20.6) to give Portion 1 (1^{st} peak, 60 mg) as a mixture of two stereoisomers, (Compound 116c; Method AG, Peak 1, 1.45 min) (S)-2-(4-(6-((R)-1-((4-isopropylphenyl)carbamoyl)pyrrolidin-2-yl)pyridin-3-yl)phenyl)propanoic acid (2^{nd} peak, 19.5 mg, 16%) as a white solid, and (Compound 116d; Method AG, Peak 2, 2.26 min) (R)-2-(4-(6-((R)-1-((4-isopropylphenyl)carbamoyl)pyrrolidin-2-yl)pyridin-3-yl)phenyl)propanoic acid (3^{rd} peak, 25.7 mg, 21%) as a white solid.

Portion 1 (60 mg) was further resolved by preparative chiral HPLC (conditions: column: CHIRALPAK AD 3*25 cm, 5 μm; mobile phase A: hexane (0.1% formic acid), mobile phase B: ethanol/methanol (1:1); Flow rate: 40 mL/min; gradient (B %): isocratic 30% B; detector: 220/254 nm; RT1 (min): 21.0; RT2 (min): 29.0) to give (Compound 116a; Method AN, Peak 1, 1.82 min) (S)-2-(4-(6-((S)-1-((4-isopropylphenyl)carbamoyl)pyrrolidin-2-yl)pyridin-3-yl)phenyl)propanoic acid (1^{st} peak, 17.1 mg, 14%) as a white solid and (Compound 116b; Method AN, Peak 2, 2.41 min) (R)-2-(4-(6-((S)-1-((4-isopropylphenyl)carbamoyl)pyrrolidin-2-yl)pyridin-3-yl)phenyl)propanoic acid (2^{nd} peak, 23.8 mg, 19%) as a white solid.

| Cmpd # | Characterization Data |
|---|---|
| 116a | LCMS (ESI) [M + H]⁺: 458.2 |
| | ¹H NMR (400 MHz, DMSO-d₆) δ 12.41 (bs, 1H), 8.80 (s, 1H), 8.21 (s, 1H), 8.01-7.98 (m, 1H), |
| | 7.65 (d, J = 8.4 Hz, 2H), 7.40-7.31 (m, 4H), 7.06 (d, J = 8.4 Hz, 2H), 5.13-5.11 (m, 1H), 3.80- |
| | 3.70 (m, 2H), 3.61-3.55 (m, 1H), 2.82-2.75 (m, 1H), 2.33-2.28 (m, 1H), 2.00-1.93 (m, 3H), |
| | 1.39 (d, J = 7.2 Hz, 3H), 1.15 (d, J = 7.2 Hz, 6H). |
| 116b | LCMS (ESI) [M + H]⁺: 458.2 |
| | ¹H NMR (400 MHz, DMSO-d₆) δ 12.42 (bs, 1H), 8.80 (s, 1H), 8.21 (s, 1H), 8.00-7.98 (m, 1H), |
| | 7.64 (d, J = 8.0 Hz, 2H), 7.40-7.31 (m, 5H), 7.06 (d, J = 8.4 Hz, 2H), 5.13-5.11 (m, 1H), 3.80- |
| | 3.69 (m, 2H), 3.61-3.55 (m, 1H), 2.82-2.75 (m, 1H), 2.35-2.28 (m, 1H), 2.00-1.93 (m, 3H), |
| | 1.38 (d, J = 7.2 Hz, 3H), 1.15 (d, J = 7.2 Hz, 6H). |
| 116c | LCMS (ESI) [M + H]⁺: 458.2 |
| | ¹H NMR (400 MHz, DMSO-d₆) δ 12.39 (bs, 1H), 8.80 (s, 1H), 8.21 (s, 1H), 8.00-7.98 (m, 1H), |
| | 7.65 (d, J = 8.0 Hz, 2H), 7.40-7.31 (m, 5H), 7.06 (d, J = 8.4 Hz, 2H), 5.13-5.11 (m, 1H), 3.80- |
| | 3.70 (m, 2H), 3.61-3.56 (m, 1H), 2.82-2.75 (m, 1H), 2.33-2.28 (m, 1H), 2.00-1.93 (m, 3H), |
| | 1.39 (d, J = 7.2 Hz, 3H), 1.15 (d, J = 7.2 Hz, 6H). |
| 116d | LCMS (ESI) [M + H]⁺: 458.2 |
| | ¹H NMR (400 MHz, DMSO-d₆) δ 12.40 (bs, 1H), 8.80 (s, 1H), 8.21 (s, 1H), 8.00-7.97 (m, 1H), |
| | 7.65 (d, J = 8.0 Hz, 2H), 7.40-7.31 (m, 5H), 7.06 (d, J = 8.4 Hz, 2H), 5.13-5.11 (m, 1H), 3.80- |
| | 3.69 (m, 2H), 3.61-3.55 (m, 1H), 2.82-2.75 (m, 1H), 2.33-2.28 (m, 1H), 2.00-1.93 (m, 3H), |
| | 1.39 (d, J = 7.2 Hz, 3H), 1.15 (d, J = 7.2 Hz, 6H). |

### Example 118: (R)-3-fluoro-4′-(2-((4-isopropylphenyl)carbamoyl)pyrrolidine-1-carboxamido)-[1,1′-biphenyl]-4-carboxylic acid

### Step 1: tert-butyl (R)-2-((4-isopropylphenyl)carbamoyl)pyrrolidine-1-carboxylate

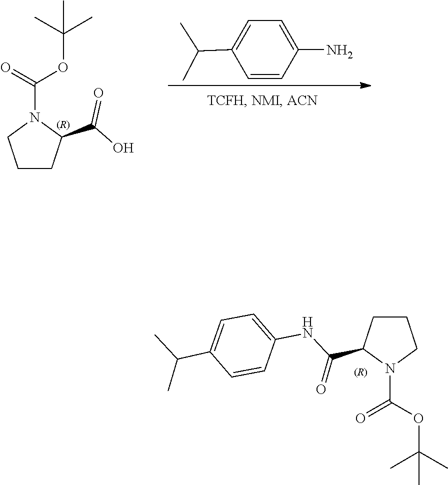

A solution of (tert-butoxycarbonyl)-D-proline (1.00 g, 4.65 mmol), 4-isopropylaniline (691 mg, 5.12 mmol), N-(chloro(dimethylamino)methylene)-N-methylmethanaminium hexafluorophosphate(V) (1.43 g, 5.11 mmol) and 1-methyl-1H-imidazole (1.14 g, 13.9 mmol) in acetonitrile (15.0 mL) was stirred at room temperature for 1 hour under nitrogen atmosphere. Upon completion, the resultant mixture was concentrated under reduced pressure. The crude residue was directly purified by silica gel column chromatography, eluted with petroleum ether/ethyl acetate (1:1), to afford tert-butyl (R)-2-((4-isopropylphenyl)carbamoyl)pyrrolidine-1-carboxylate (1.00 g, 64%) as a white solid. LCMS [M+H]⁺: 333

### Step 2: (R)—N-(4-isopropylphenyl)pyrrolidine-2-carboxamide

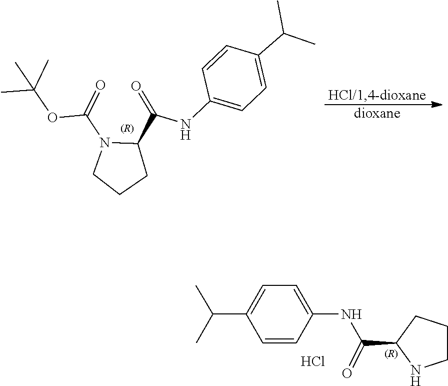

To a mixture of tert-butyl (R)-2-((4-isopropylphenyl)carbamoyl)pyrrolidine-1-carboxylate (1.00 g, 3.00 mmol) in 1,4-dioxane (2.0 mL) was added HCl (4M in 1,4-dioxane, 10.0 mL) dropwise at 0° C. The mixture was warmed to room temperature and stirred for 1 hour. Upon completion, the reaction mixture was concentrated under reduced pressure to afford (R)—N-(4-isopropylphenyl)pyrrolidine-2-carboxamide (1.20 g, crude) as a yellow solid, which was used in the next step without further purification. LCMS [M+H]⁺: 233

### Step 3: tert-butyl 4′-amino-3-fluoro-[1,1′-biphenyl]-4-carboxylate

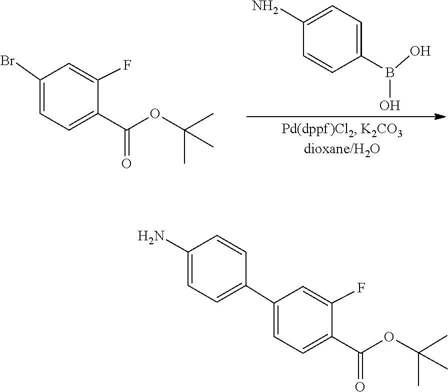 [SMILES: CC(C)(C)OC(=O)c1ccc(Br)cc1F ; Nc1ccc(B(O)O)cc1 ; CC(C)(C)OC(=O)c1ccc(-c2ccc(N)cc2)cc1F]

To a mixture of tert-butyl 4-bromo-2-fluorobenzoate (150 mg, 0.545 mmol) in 1,4-dioxane (4.0 mL) were added water (1.0 mL), K₂CO₃ (151 mg, 1.10 mmol), (4-aminophenyl)boronic acid (71.0 mg, 0.50 mmol), and Pd(dppf)Cl₂ (39.0 mg, 54.0 μmol) at room temperature under nitrogen atmosphere. The resultant mixture was stirred at 80° C. for 1 hour. Upon completion, the mixture was cooled to room temperature and diluted with water (8 mL). The resultant mixture was extracted with ethyl acetate (3×15 mL). The combined organic layers were dried over anhydrous Na₂SO₄, filtered, and concentrated under reduced pressure. The crude residue was purified by silica gel column chromatography, eluted with petroleum ether/ethyl acetate (4:1), to afford tert-butyl 4′-amino-3-fluoro-[1,1′-biphenyl]-4-carboxylate (130 mg, 82%) as a yellow solid. LCMS [M+H]⁺: 288

### Step 4: tert-butyl (R)-3-fluoro-4′-(2-((4-isopropylphenyl)carbamoyl)pyrrolidine-1-carboxamido)-[1,1′-biphenyl]-4-carboxylate

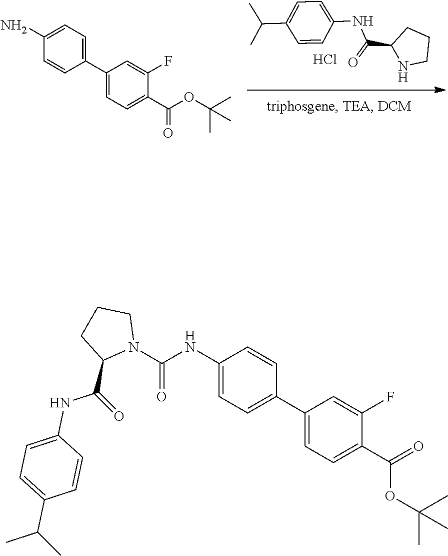 [SMILES: CC(C)(C)OC(=O)c1ccc(-c2ccc(N)cc2)cc1F ; CC(C)c1ccc(NC(=O)[C@H]2CCCN2)cc1 ; Cl ; CC(C)c1ccc(NC(=O)[C@H]2CCCN2C(=O)Nc2ccc(-c3ccc(C(=O)OC(C)(C)C)c(F)c3)cc2)cc1]

To a solution of tert-butyl 4′-amino-3-fluoro-[1,1′-biphenyl]-4-carboxylate (109 mg, 0.380 mmol) and triethylamine (112 mg, 1.11 mmol) in DCM (5.0 mL) was added triphosgene (38 mg, 0.128 mmol) dropwise at 0° C. After 30 minutes, the reaction was warmed to room temperature and (R)—N-(4-isopropylphenyl)pyrrolidine-2-carboxamide hydrochloride (102 mg, 0.381 mmol) was added dropwise over 2 minutes. After stirring for an additional 2 hours, the reaction mixture was cooled to 0° C. and was quenched with ice water. The resultant mixture was extracted with ethyl acetate (3×15 mL). The combined organic layers were dried over anhydrous Na₂SO₄, filtered, and concentrated under reduced pressure. The crude residue was purified by silica gel column chromatography, eluted with petroleum ether/ethyl acetate (1:1), to afford tert-butyl (R)-3-fluoro-4′-(2-((4-isopropylphenyl)carbamoyl)pyrrolidine-1-carboxamido)-[1,1′-biphenyl]-4-carboxylate (110 mg, 60%) as a yellow solid. LCMS [M+H]⁺: 546.0

### Step 5: (R)-3-fluoro-4′-(2-((4-isopropylphenyl)carbamoyl)pyrrolidine-1-carboxamido)-[1,1′-biphenyl]-4-carboxylic acid

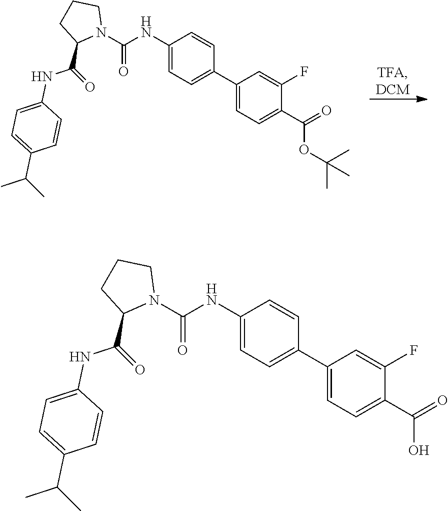

To a mixture of tert-butyl (R)-3-fluoro-4′-(2-((4-isopropylphenyl)carbamoyl)pyrrolidine-1-carboxamido)-[1,1′-biphenyl]-4-carboxylate (100 mg, 0.183 mmol) in DCM (3.0 mL) added TFA (1.0 mL) dropwise at 0° C. The resultant mixture was warmed to room temperature and stirred for 1 hours. Upon completion, the mixture was concentrated under reduced pressure. The crude residue was purified by Prep-HPLC (conditions: column: Xselect CSH Prep C18, 30*150 mm 5 μm; mobile Phase A: water (0.1% formic acid), mobile phase B: acetonitrile; Flow rate: 60 mL/min; gradient (B %): isocratic 43% to 63% B in 10 min; Detector: 254 nm/220 nm; RT (min): 10.4 min) to afford (R)-3-fluoro-4′-(2-((4-isopropylphenyl)carbamoyl)pyrrolidine-1-carboxamido)-[1,1′-biphenyl]-4-carboxylic acid (18.5 mg, 21%) as a white solid.

| Cmpd | |
|---|---|
| # | Characterization Data |
| 118 | LCMS (ESI) [M + H]⁺: 490.1 |
| | ¹H NMR (400 MHz, DMSO-d₆) δ 13.13 (bs, 1H), |
| | 9.91 (s, 1H), 8.47 (s, 1H), 7.89 (t, J = 8.0 Hz, 1H), 7.70-7.65 |
| | (m, 4H), 7.59 (d, J = 10.4 Hz, 2H), 7.51 (d, J = 8.4 Hz, 2H), |
| | 7.16 (d, J = 8.4 Hz, 2H), 4.49-4.46 (m, 1H), 3.70-3.65 (m, 1H), |
| | 3.57-3.52 (m, 1H), 2.86-2.79 (m, 1H), 2.21-2.14 (m, 1H), 2.04- |
| | 1.91 (m, 3H), 1.17 (d, J = 7.2 Hz, 6H). |

### Example 119: (R)-5-(4-(2-((3-fluoro-4-isopropylphenyl)carbamoyl)pyrrolidine-1-carboxamido)phenyl)-6-methylpicolinic acid

### Step 1: tert-butyl (R)-2-((3-fluoro-4-isopropylphenyl)carbamoyl)pyrrolidine-1-carboxylate

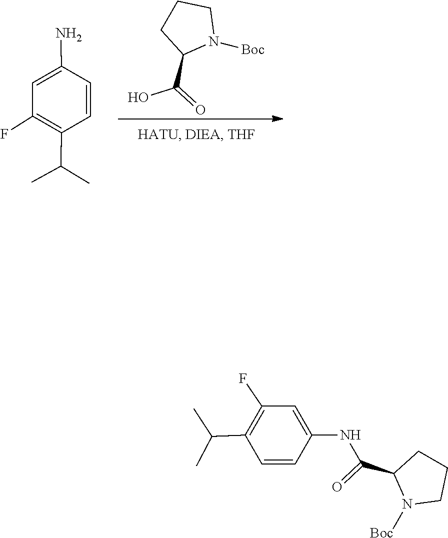 [SMILES: CC(C)c1ccc(N)cc1F ; CC(C)(C)OC(=O)N1CCC[C@@H]1C(=O)O ; CC(C)c1ccc(NC(=O)[C@H]2CCCN2C(=O)OC(C)(C)C)cc1F]

HATU (596 mg, 1.57 mmol) and DIEA (186 mg, 1.44 mmol) were added to a solution of (tert-butoxycarbonyl)-D-proline (309 mg, 1.43 mmol) in THF (6.0 mL) at 0° C. After 30 minutes, 3-fluoro-4-isopropylaniline (200 mg, 1.31 mmol) was added dropwise. The reaction was warmed to room temperature and was allowed to stir for an additional 2 hours. Upon completion, the reaction mixture was concentrated under reduced pressure. The crude residue was purified by reversed-phase flash chromatography (conditions: column: C18 silica gel; mobile phase A: water (0.1% formic acid), mobile phase B: acetonitrile; gradient (B %): 0% to 100% over 25 min; detector: 254 nm) to afford tert-butyl (R)-2-((3-fluoro-4-isopropylphenyl)carbamoyl)pyrrolidine-1-carboxylate (260 mg, 65%) as a pale-yellow solid. LCMS [M+H]⁺: 351.0

### Step 2: (R)—N-(3-fluoro-4-isopropylphenyl)pyrrolidine-2-carboxamide

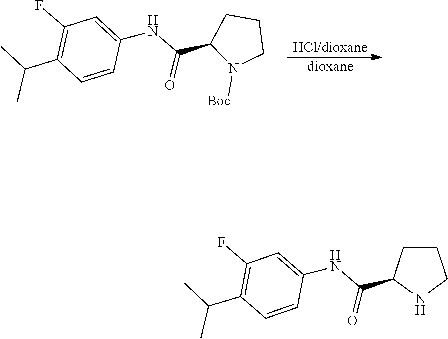 [SMILES: CC(C)c1ccc(NC(=O)[C@H]2CCCN2C(=O)OC(C)(C)C)cc1F ; CC(C)c1ccc(NC(=O)[C@H]2CCCN2)cc1F]

To a mixture of tert-butyl (R)-2-((3-fluoro-4-isopropylphenyl)carbamoyl)pyrrolidine-1-carboxylate (250 mg, 0.713 mmol) in dioxane (1.0 mL) at 0° C. was added HCl (4M in 1,4-dioxane 5.0 mL) dropwise. The reaction mixture was warmed to room temperature and was allowed to stir for 2 hours. Upon completion, the resultant mixture was concentrated under reduced pressure to afford (R)—N-(3-fluoro-4-isopropylphenyl)pyrrolidine-2-carboxamide (200 mg, crude) as a yellow solid, which was used in the next step without further purification. LCMS [M+H]⁺: 251.0

### Step 3: methyl (R)-5-(4-(2-((3-fluoro-4-isopropylphenyl)carbamoyl)pyrrolidine-1-carboxamido)phenyl)-6-methylpicolinate

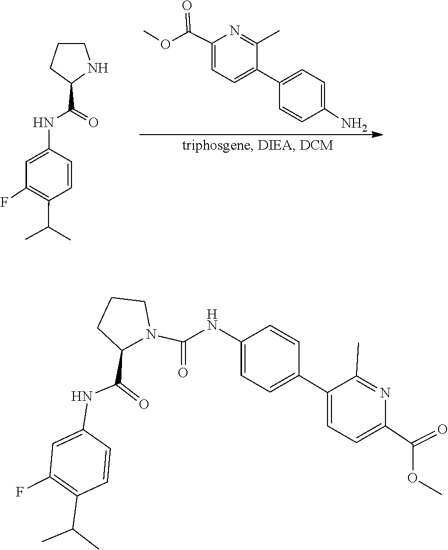 [SMILES: CC(C)c1ccc(NC(=O)[C@H]2CCCN2)cc1F ; COC(=O)c1ccc(-c2ccc(N)cc2)c(C)n1 ; COC(=O)c1ccc(-c2ccc(NC(=O)N3CCC[C@@H]3C(=O)Nc3ccc(C(C)C)c(F)c3)cc2)c(C)n1]

To a mixture of methyl 5-(4-aminophenyl)-6-methylpicolinate (184 mg, 0.760 mmol) in DCM (6.0 mL) was added DIEA (491 mg, 3.81 mmol) followed by the dropwise addition of triphosgene (90.0 mg, 0.303 mmol) at 0° C. After 30 minutes, (R)—N-(3-fluoro-4-isopropylphenyl)pyrrolidine-2-carboxamide (190 mg, 0.759 mmol) was added dropwise over 2 minutes. The mixture was warmed to room temperature and was allowed to stir for an additional 2 hours. Upon completion, the reaction mixture was quenched with ice water. The mixture was extracted with ethyl acetate (3×20 mL). The combined organic layers were dried over anhydrous Na₂SO₄, filtered, and concentrated under reduced pressure. The crude residue was purified by silica gel column chromatography, eluted with CH₂Cl₂/methanol (5:1), to afford methyl (R)-5-(4-(2-((3-fluoro-4-isopropylphenyl)carbamoyl)pyrrolidine-1-carboxamido)phenyl)-6-methylpicolinate (150 mg, 38%) as a pale-yellow solid. LCMS [M+H]⁺: 519.0

### Step 4: (R)-5-(4-(2-((3-fluoro-4-isopropylphenyl)carbamoyl)pyrrolidine-1-carboxamido)phenyl)-6-methylpicolinic acid

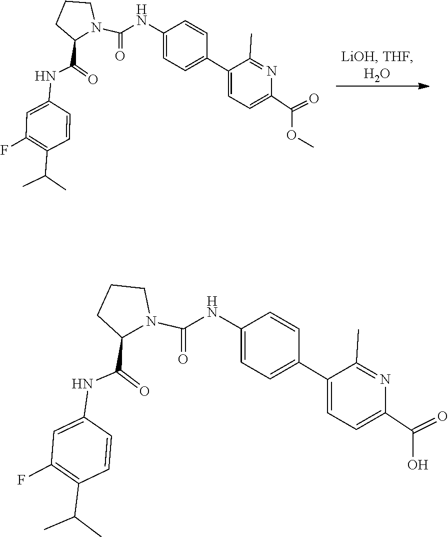 [SMILES: COC(=O)c1ccc(-c2ccc(NC(=O)N3CCC[C@@H]3C(=O)Nc3ccc(C(C)C)c(F)c3)cc2)c(C)n1 ; Cc1nc(C(=O)O)ccc1-c1ccc(NC(=O)N2CCC[C@@H]2C(=O)Nc2ccc(C(C)C)c(F)c2)cc1]

A solution of methyl (R)-5-(4-(2-((3-fluoro-4-isopropylphenyl)carbamoyl)pyrrolidine-1-carboxamido)phenyl)-6-methylpicolinate (110 mg, 0.212 mmol) and LiOH monohydrate (26.0 mg, 0.630 mmol) in THF (2.5 mL) and water (2.5 mL) was stirred at room temperature for 2 hours. Upon completion, the mixture was acidified to pH ˜3 with HCl (1 M). The resultant mixture was concentrated under reduced pressure. The crude residue purified by Prep-HPLC (conditions: column: Xselect CSH Prep C18, 30*150 mm 5 μm; mobile phase A: water (0.1% formic acid), mobile phase B: acetonitrile; Flow rate: 60 mL/min; gradient (B %): 30% B to 50% B in 15 min; Detector: 254 nm/220 nm; RT (min): 9.63) to afford (R)-5-(4-(2-((3-fluoro-4-isopropylphenyl)carbamoyl)pyrrolidine-1-carboxamido)phenyl)-6-methylpicolinic acid (39.3 mg, 37%) as a white solid.

| Cmpd | |
|---|---|
| # | Characterization Data |
| 119 | LCMS (ESI) [M + H]⁺: 505.3 |
| | ¹H NMR (400 MHz, DMSO-d₆) δ 12.89 (bs, 1H), |
| | 10.12 (s, 1H), 8.46 (s, 1H), 7.90 (d, J = 8.0 Hz, 1H), 7.75 (d, |
| | J = 8.0 Hz, 1H), 7.64 (d, J = 8.8 Hz, 2H), 7.60-7.53 (m, 1H), |
| | 7.32 (d, J = 8.8 Hz, 2H), 7.29-7.23 (m, 2H), 4.47-4.44 (m, 1H), |
| | 3.69-3.65 (m, 1H), 3.57-3.54 (m, 1H), 3.1-3.08 (m, 1H), 2.52- |
| | 2.51(m, 3H), 2.20-2.17 (m, 1H), 2.04-1.91 (m, 3H), 1.18 (d, J = |
| | 6.8 Hz, 6H). |

### Example 121: 4-(5-fluoro-6-((2S,4R)-1-((4-isopropylphenyl)carbamoyl)-4-methylpyrrolidin-2-yl)pyridin-3-yl)benzoic acid and 4-(5-fluoro-6-((2R,4R)-1-((4-isopropylphenyl)carbamoyl)-4-methylpyrrolidin-2-yl)pyridin-3-yl)benzoic acid

### Step 1: tert-butyl (4R)-2-(5-(4-(tert-butoxycarbonyl)phenyl)-3-fluoropyridin-2-yl)-4-methylpyrrolidine-1-carboxylate

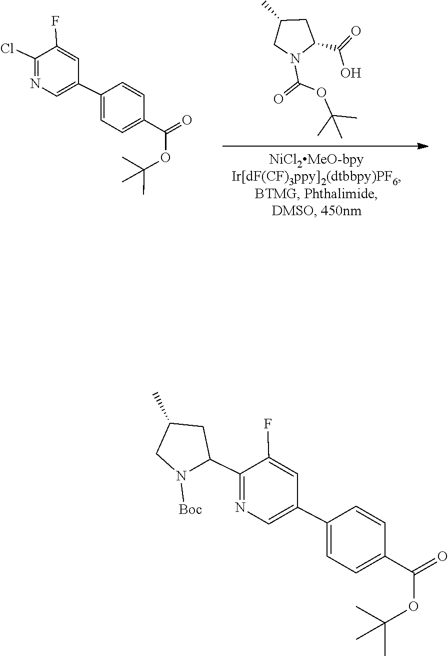 [SMILES: CC(C)(C)OC(=O)c1ccc(-c2cnc(Cl)c(F)c2)cc1 ; C[C@@H]1C[C@H](C(=O)O)N(C(=O)OC(C)(C)C)C1 ; C[C@@H]1CC(c2ncc(-c3ccc(C(=O)OC(C)(C)C)cc3)cc2F)N(C(=O)OC(C)(C)C)C1]

A solution of tert-butyl 4-(6-chloro-5-fluoropyridin-3-yl)benzoate (300 mg, 0.975 mmol), (2R,4R)-1-(tert-butoxycarbonyl)-4-methylpyrrolidine-2-carboxylic acid (335 mg, 1.46 mmol), nickel(II) chloride ethylene glycol dimethyl ether complex (21 mg, 95.9 μmol), 4,4′-dimethoxy-2,2′-bipyridine (25 mg, 0.116 mmol), N″-tert-butyl-N,N,N′,N′-tetramethylguanidine (250 mg, 1.50 mmol), Ir[dF(CF₃)ppy]₂(dtbpy))PF₆ (22 mg, 19.0 μmol), and 2,3-dihydro-1H-isoindole-1,3-dione (143 mg, 0.971 mmol) in DMSO (5.0 mL) was stirred at room temperature under nitrogen atmosphere with irradiation at 450 nm using blue LEDs for 24 hours. Upon completion, the resultant mixture was diluted with water and extracted with ethyl acetate (3×20 mL). The combined organic layers were washed with brine, dried over anhydrous Na₂SO₄, filtered and concentrated under reduced pressure. The crude residue was purified by silica gel column chromatography, eluted with petroleum ether/ethyl acetate (1:1), to afford tert-butyl (4R)-2-(5-(4-(tert-butoxycarbonyl)phenyl)-3-fluoropyridin-2-yl)-4-methylpyrrolidine-1-carboxylate (200 mg, 45%) as a pale-yellow solid. LCMS [M+H]⁺: 457.0

### Step 2: 4-(5-fluoro-6-((4R)-4-methylpyrrolidin-2-yl)pyridin-3-yl)benzoic acid

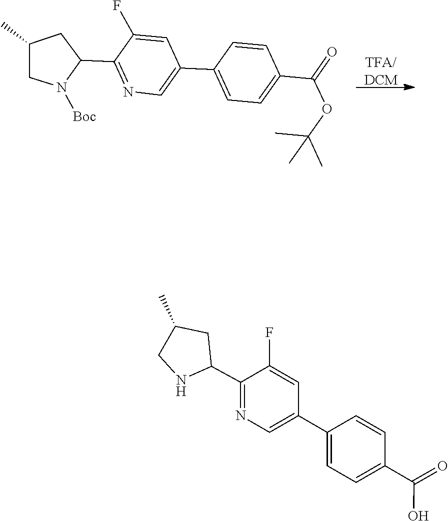 [SMILES: C[C@@H]1CC(c2ncc(-c3ccc(C(=O)OC(C)(C)C)cc3)cc2F)N(C(=O)OC(C)(C)C)C1 ; C[C@H]1CNC(c2ncc(-c3ccc(C(=O)O)cc3)cc2F)C1]

A solution of tert-butyl (4R)-2-(5-(4-(tert-butoxycarbonyl)phenyl)-3-fluoropyridin-2-yl)-4-methylpyrrolidine-1-carboxylate (200 mg, 0.438 mmol) in TFA (3.0 mL) and DCM (1.0 mL) was stirred at room temperature for 2 hours. Upon completion, the resultant mixture was concentrated under reduced pressure to afford 4-(5-fluoro-6-((4R)-4-methylpyrrolidin-2-yl)pyridin-3-yl)benzoic acid (120 mg, crude) as a pale-yellow solid, which was used in the next step without further purification. LCMS [M+H]⁺: 301.0

### Step 3: 4-(5-fluoro-6-((4R)-1-((4-isopropylphenyl)carbamoyl)-4-methylpyrrolidin-2-yl)pyridin-3-yl)benzoic acid

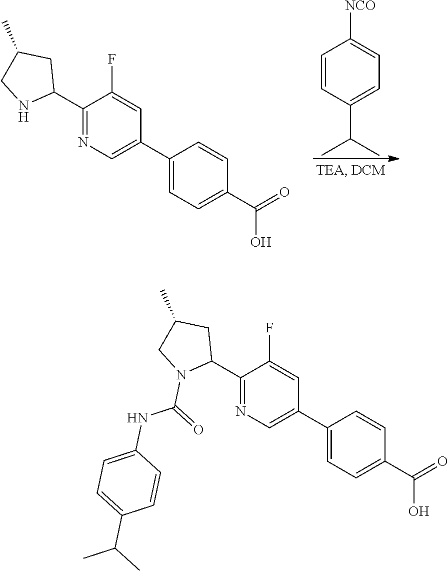

To a mixture of 4-(5-fluoro-6-((4R)-4-methylpyrrolidin-2-yl)pyridin-3-yl)benzoic acid (100 mg, 0.300 mmol) in DCM (5.0 mL) was added triethylamine (34.0 mg, 0.336 mmol), followed by the dropwise addition of 1-isocyanato-4-isopropylbenzene (54 mg, 0.335 mmol) at 0° C. The resultant mixture was warmed to room temperature and stirred for 2 hours. Upon completion, the mixture was cooled to 0° C., quenched with ice water, and extracted with ethyl acetate (3×15 mL). The combined organic layers were dried over anhydrous Na₂SO₄, filtered, and concentrated under reduced pressure. The crude residue was purified by reversed-phase flash chromatography (conditions: column: C18 silica gel; mobile phase A: water (0.1% formic acid), Mobile phase B: acetonitrile; gradient (B %): 0% to 100% over 25 min; detector: 254 nm) to afford 4-(5-fluoro-6-((4R)-1-((4-isopropylphenyl)carbamoyl)-4-methylpyrrolidin-2-yl)pyridin-3-yl)benzoic acid (80 mg, 52%) as a yellow solid. LCMS [M+H]⁺: 462.0

### Step 3: 4-(5-fluoro-6-((2S,4R)-1-((4-isopropylphenyl)carbamoyl)-4-methylpyrrolidin-2-yl)pyridin-3-yl)benzoic acid and 4-(5-fluoro-6-((2R,4R)-1-((4-isopropylphenyl)carbamoyl)-4-methylpyrrolidin-2-yl)pyridin-3-yl)benzoic acid

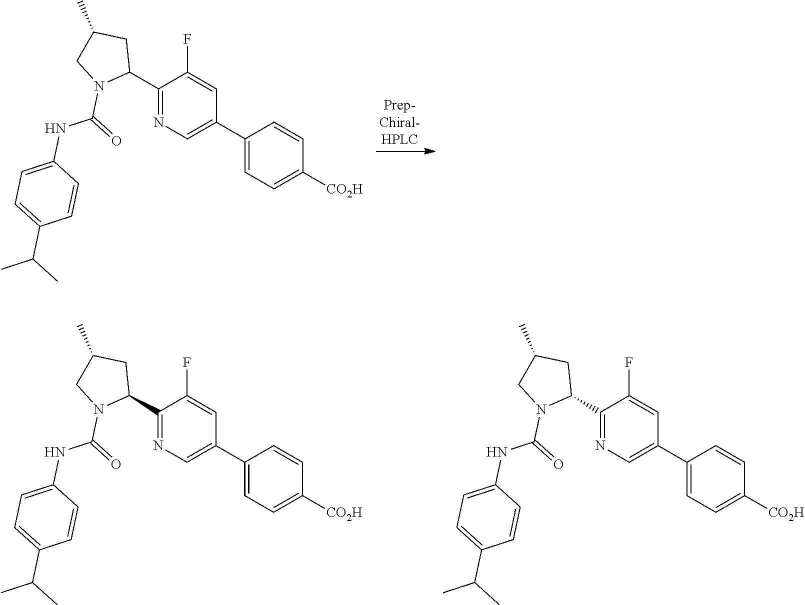 [SMILES: CC(C)c1ccc(NC(=O)N2C[C@H](C)CC2c2ncc(-c3ccc(C(=O)O)cc3)cc2F)cc1 ; CC(C)c1ccc(NC(=O)N2C[C@H](C)C[C@H]2c2ncc(-c3ccc(C(=O)O)cc3)cc2F)cc1 ; CC(C)c1ccc(NC(=O)N2C[C@H](C)C[C@@H]2c2ncc(-c3ccc(C(=O)O)cc3)cc2F)cc1]

4-(5-fluoro-6-((4R)-1-((4-isopropylphenyl)carbamoyl)-4-methylpyrrolidin-2-yl)pyridin-3-yl)benzoic acid (77 mg) was resolved by preparative chiral HPLC (conditions: column: CHIRALPAK IK 3*25 cm, 5 μm; mobile phase A: hexane (0.1% formic acid), mobile phase B: ethanol; Flow rate: 40 mL/min; gradient (B %): isocratic 20% B; detector: 220/254 nm; RT1(min): 13.8; RT2 (min): 17.0) to afford first peak (Compound 121a; Method P, Peak 1, 1.52 min) 4-(5-fluoro-6-((2S,4R)-1-((4-isopropylphenyl)carbamoyl)-4-methylpyrrolidin-2-yl)pyridin-3-yl)benzoic acid (43.3 mg, 56%) as a white solid and second peak (Compound 121b; Method P, Peak 2, 1.92 min) 4-(5-fluoro-6-((2R,4R)-1-((4-isopropylphenyl)carbamoyl)-4-methylpyrrolidin-2-yl)pyridin-3-yl)benzoic acid (12.9 mg, 17%) as a white solid.

| Cmpd # | Characterization Data |
|---|---|
| 121a | LCMS (ESI) [M + H]⁺: 462.1 |
| | ¹H NMR (400 MHz, DMSO-d₆) δ 13.09 (bs, 1H), 8.73 (s, 1H), 8.11-8.02 (m, 4H), 7.88 (d, J = 8.4 |
| | Hz, 2H), 7.43 (d, J = 8.4 Hz, 2H), 7.04 (d, J = 8.4 Hz, 2H), 5.39-5.38 (m, 1H), 3.96-3.92 (m, |
| | 1H), 3.17 (t, J = 8.6 Hz, 1H), 2.81-2.74 (m, 1H), 2.67-2.59 (m, 1H), 2.06-1.98 (m, 2H), 1.14 |
| | (d, J = 6.8 Hz, 6H), 1.09 (d, J = 6.8 Hz, 3H). |
| 121b | LCMS (ESI) [M + H]⁺: 462.1 |
| | ¹H NMR (400 MHz, DMSO-d₆) δ 13.11 (bs, 1H), 8.73 (s, 1H), 8.17 (s, 1H), 8.03-8.00 (m, 3H), |
| | 7.86 (d, J = 8.4 Hz, 2H), 7.29 (d, J = 8.9 Hz, 2H), 7.02 (d, J = 8.8 Hz, 2H), 5.27-5.23 (m, 1H), |
| | 3.90-3.86 (m, 1H), 3.25-3.21 (m, 1H), 2.80-2.73 (m, 1H), 2.51-2.49 (m, 2H), 1.62-1.59 (m, |
| | 1H), 1.14-1.09 (m, 9H). |

### Example 122: 4-(5-fluoro-6-((2S,4S)-1-((4-isopropylphenyl)carbamoyl)-4-methylpyrrolidin-2-yl)pyridin-3-yl)benzoic acid & 4-(5-fluoro-6-((2R,4S)-1-((4-isopropylphenyl)carbamoyl)-4-methylpyrrolidin-2-yl)pyridin-3-yl)benzoic acid

### Step 1: tert-butyl (4S)-2-(5-(4-(tert-butoxycarbonyl)phenyl)-3-fluoropyridin-2-yl)-4-methylpyrrolidine-1-carboxylate

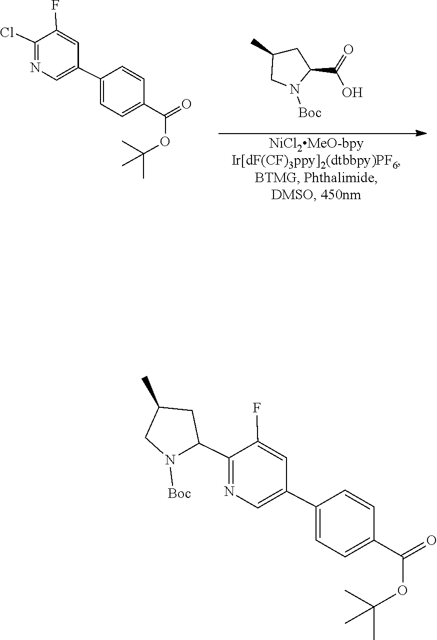 [SMILES: CC(C)(C)OC(=O)c1ccc(-c2cnc(Cl)c(F)c2)cc1 ; C[C@H]1C[C@@H](C(=O)O)N(C(=O)OC(C)(C)C)C1 ; C[C@H]1CC(c2ncc(-c3ccc(C(=O)OC(C)(C)C)cc3)cc2F)N(C(=O)OC(C)(C)C)C1]

A solution of tert-butyl 4-(6-chloro-5-fluoropyridin-3-yl)benzoate (300 mg, 0.977 mmol), (2S,4S)-1-(tert-butoxycarbonyl)-4-methylpyrrolidine-2-carboxylic acid (335 mg, 1.50 mmol), Nickel(II) chloride ethylene glycol dimethyl ether complex (21.0 mg, 98.0 μmol), 4,4′-dimethoxy-2,2′-bipyridine (25.0 mg, 0.116 mmol), N″-tert-butyl-N,N,N′,N′-tetramethylguanidine (250 mg, 1.50 mmol), Ir[dF(CF₃)ppy]₂(dtbpy))PF₆ (22 mg, 19.0 μmol), and 2,3-dihydro-1H-isoindole-1,3-dione (143 mg, 0.973 mmol) in DMSO (5.0 mL) at room temperature was stirred under 450 nm blue LED irradiation for 24 hours. Upon completion, the resultant mixture was diluted with water and extracted with ethyl acetate (3×15 mL). The combined organic layers were washed with brine, dried over anhydrous Na₂SO₄, filtered, and concentrated under reduced pressure. The crude residue was purified by silica gel column chromatography, eluted with petroleum ether/ethyl acetate (1:1), to afford tert-butyl (4S)-2-(5-(4-(tert-butoxycarbonyl)phenyl)-3-fluoropyridin-2-yl)-4-methylpyrrolidine-1-carboxylate (240 mg, 54%) as a pale-yellow solid. LCMS [M+H]⁺: 457.0

### Step 2: 4-(5-fluoro-6-((4S)-4-methylpyrrolidin-2-yl)pyridin-3-yl)benzoic acid

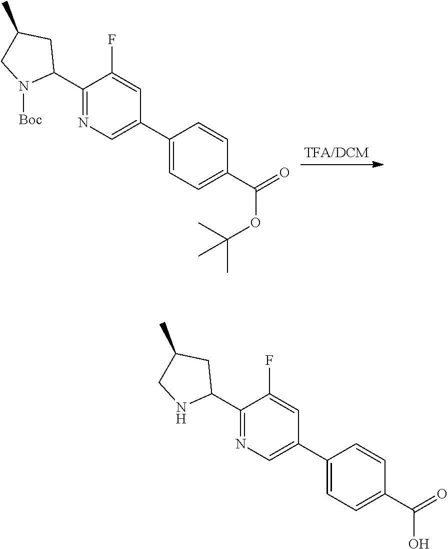 [SMILES: C[C@H]1CC(c2ncc(-c3ccc(C(=O)OC(C)(C)C)cc3)cc2F)N(C(=O)OC(C)(C)C)C1 ; C[C@@H]1CNC(c2ncc(-c3ccc(C(=O)O)cc3)cc2F)C1]

A solution of tert-butyl (4S)-2-(5-(4-(tert-butoxycarbonyl)phenyl)-3-fluoropyridin-2-yl)-4-methylpyrrolidine-1-carboxylate (230 mg, 0.504 mmol) in TFA (6.0 mL) and DCM (2.0 mL) was stirred at room temperature for 2 hours. Upon completion, the resulting mixture was concentrated under reduced pressure to afford 4-(5-fluoro-6-((4S)-4-methylpyrrolidin-2-yl)pyridin-3-yl)benzoic acid (150 mg, crude) as a pale-yellow solid, which was used directly in the next step without further purification. LCMS [M+H]⁺: 301

### Step 3: 4-(5-fluoro-6-((4S)-1-((4-isopropylphenyl)carbamoyl)-4-methylpyrrolidin-2-yl)pyridin-3-yl)benzoic acid

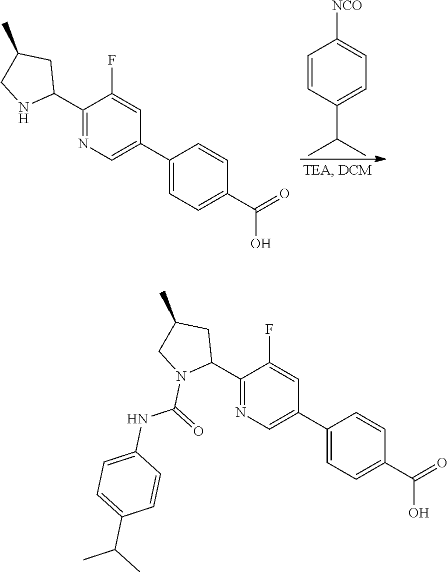

To 4-(5-fluoro-6-((4S)-4-methylpyrrolidin-2-yl)pyridin-3-yl)benzoic acid (140 mg, 0.467 mmol) in DCM (6.0 mL) was added triethylamine (47.0 mg, 0.465 mmol) followed by the dropwise addition of 1-isocyanato-4-isopropylbenzene (75.0 mg, 0.466 mmol) at room temperature under nitrogen atmosphere. The resultant mixture was stirred at room temperature for 2 hours. Upon completion, the reaction mixture was cooled in an ice bath and quenched with ice water. The resultant mixture was then extracted with ethyl acetate (3×20 mL). The combined organic layers were dried over anhydrous Na₂SO₄, filtered, and concentrated under reduced pressure. The crude residue was purified by reversed-phase flash chromatography (conditions: column: C18 silica gel; mobile phase A, water (0.1% formic acid), mobile phase B: acetonitrile; gradient: 0% to 100% over 25 min; detector: 254 nm) to afford 4-(5-fluoro-6-((4S)-1-((4-isopropylphenyl)carbamoyl)-4-methylpyrrolidin-2-yl)pyridin-3-yl)benzoic acid (120 mg, 56%) as a yellow solid. LCMS [M+H]⁺: 462.0

### Step 3: 4-(5-fluoro-6-((2S,4S)-1-((4-isopropylphenyl)carbamoyl)-4-methylpyrrolidin-2-yl)pyridin-3-yl)benzoic acid & 4-(5-fluoro-6-((2R,4S)-1-((4-isopropylphenyl)carbamoyl)-4-methylpyrrolidin-2-yl)pyridin-3-yl)benzoic acid

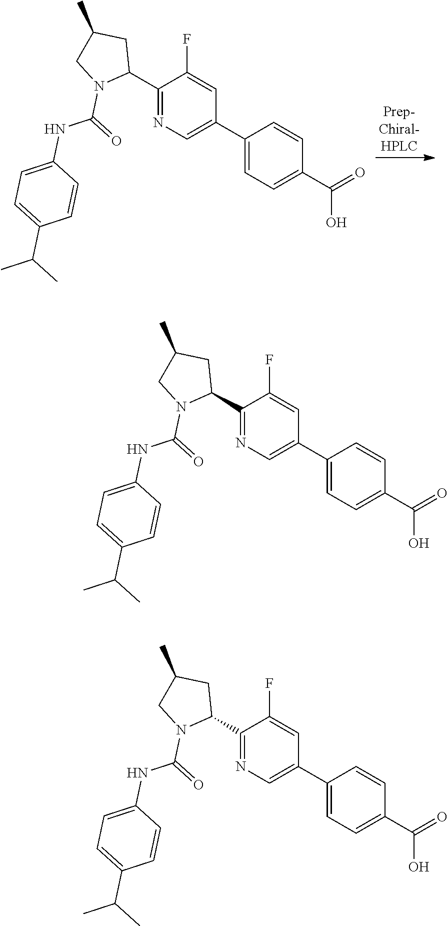 [SMILES: CC(C)c1ccc(NC(=O)N2C[C@@H](C)CC2c2ncc(-c3ccc(C(=O)O)cc3)cc2F)cc1 ; CC(C)c1ccc(NC(=O)N2C[C@@H](C)C[C@H]2c2ncc(-c3ccc(C(=O)O)cc3)cc2F)cc1 ; CC(C)c1ccc(NC(=O)N2C[C@@H](C)C[C@@H]2c2ncc(-c3ccc(C(=O)O)cc3)cc2F)cc1]

4-(5-fluoro-6-((4S)-1-((4-isopropylphenyl)carbamoyl)-4-methylpyrrolidin-2-yl)pyridin-3-yl)benzoic acid (117 mg) was resolved by preparative chiral HPLC (column: CHIRAL ART Cellulose-SC, 3*25 cm, 5 μm; mobile phase A: water (0.1% formic acid), mobile phase B: ethanol; Flow rate: 40 mL/min; gradient (B %): isocratic 30% B; Detector: 220/254 nm; RT1 (min): 7.0; RT2 (min): 11.7) to afford first peak (Compound 122a; Method W, Peak 1, 0.91 min) 4-(5-fluoro-6-((2S,4S)-1-((4-isopropylphenyl)carbamoyl)-4-methylpyrrolidin-2-yl)pyridin-3-yl)benzoic acid (25.2 mg, 22%) as a white solid, and second peak (Compound 122b; Method W, Peak 2, 1.76 min) 4-(5-fluoro-6-((2R,4S)-1-((4-isopropylphenyl)carbamoyl)-4-methylpyrrolidin-2-yl)pyridin-3-yl)benzoic acid (71.5 mg, 61%) as a white solid.

| Cmpd # | Characterization Data |
|---|---|
| 122a | LCMS (ESI) [M + H]⁺: 462.1 |
| | ¹H NMR (400 MHz, DMSO-d₆) δ 13.06 (bs, 1H), 8.73 (s, 1H), 8.17 (s, 1H), 8.05-8.01 (m, 3H), |
| | 7.88 (d, J = 8.4 Hz, 2H), 7.30 (d, J = 8.4 Hz, 2H), 7.02 (d, J = 8.8 Hz, 2H), 5.27-5.23 (m, 1H), |
| | 3.90-3.86 (m, 1H), 3.32-3.20 (m, 1H), 2.80-2.73 (m, 1H), 2.52-2.43 (m, 2H), 1.64-1.59 (m, |
| | 1H), 1.15-1.09 (m, 9H). |
| 122b | LCMS (ESI) [M + H]⁺: 462.2 |
| | ¹H NMR (400 MHz, DMSO-d₆) δ 13.07 (bs, 1H), 8.73 (s, 1H), 8.11 (s, 1H), 8.08-8.02 (m, 3H), |
| | 7.89 (d, J = 8.0 Hz, 2H), 7.44 (d, J = 8.4 Hz, 2H), 7.04 (d, J = 8.4 Hz, 2H), 5.40-5.38 (m, 1H), |
| | 3.96-3.92 (m, 1H), 3.19-3.15 (m, 1H), 2.81-2.74 (m, 1H), 2.67-2.59 (m, 1H), 2.06-1.97 (m, |
| | 2H), 1.14 (d, J = 6.8 Hz, 6H), 1.09 (d, J = 6.8 Hz, 3H). |

### Example 124: 4-(6-((2S,3S)-1-((4-isopropylphenyl)carbamoyl)-3-methylpyrrolidin-2-yl)pyridin-3-yl)benzoic acid & 4-(6-((2R,3R)-1-((4-isopropylphenyl)carbamoyl)-3-methylpyrrolidin-2-yl)pyridin-3-yl)benzoic acid

### Step 1: 1-(tert-butoxycarbonyl)-3-methyl-1H-pyrrole-2-carboxylic acid

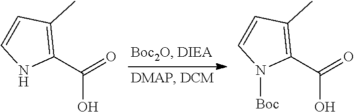

To a mixture of 3-methyl-1H-pyrrole-2-carboxylic acid (2.00 g, 16.0 mmol) and Boc₂O (3.84 g, 17.6 mmol) in DCM (30 mL) at 0° C. were added DIEA (6.20 g, 48.0 mmol) and DMAP (0.590 g, 4.80 mmol). The resultant mixture was warmed to room temperature and was allowed to stir for 16 hours. Upon completion, the mixture was concentrated under reduced pressure. The crude residue was directly purified by reversed-phase flash chromatography (conditions: column: C18 silica gel; mobile phase A: water (0.1% formic acid), mobile phase B: acetonitrile; gradient (B %): 10% to 40% gradient in 20 min; detector: 254 nm) to afford 1-(tert-butoxycarbonyl)-3-methyl-1H-pyrrole-2-carboxylic acid (1.00 g, 28%) as an off white solid. LCMS [M+H]⁺: 226.1

### Step 2: 1-(tert-butoxycarbonyl)-3-methylpyrrolidine-2-carboxylic acid

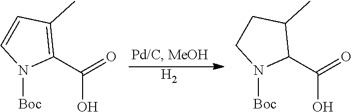 [SMILES: Cc1ccn(C(=O)OC(C)(C)C)c1C(=O)O ; "H2" ; CC1CCN(C(=O)OC(C)(C)C)C1C(=O)O]

To a mixture of 1-(tert-butoxycarbonyl)-3-methyl-1H-pyrrole-2-carboxylic acid (1.00 g, 4.40 mmol) in methanol (15.0 mL) under nitrogen atmosphere was added portion-wise Pd/C (0.940 g, 5.30 mmol, 10 wt %). The resultant mixture was stirred at 50° C. for 16 hours under 50 atm of hydrogen atmosphere. Upon completion, the mixture was filtered through celite. The filter cake was washed with methanol (3×20 mL). The filtrate was then concentrated under reduced pressure to afford 1-(tert-butoxycarbonyl)-3-methylpyrrolidine-2-carboxylic acid (810 mg, crude) as a yellow oil, which was used in the next step without further purification. LCMS [M+H]⁺: 230.1

### Step 3: tert-butyl 2-(5-(4-(tert-butoxycarbonyl)phenyl)pyridin-2-yl)-3-methylpyrrolidine-1-carboxylate

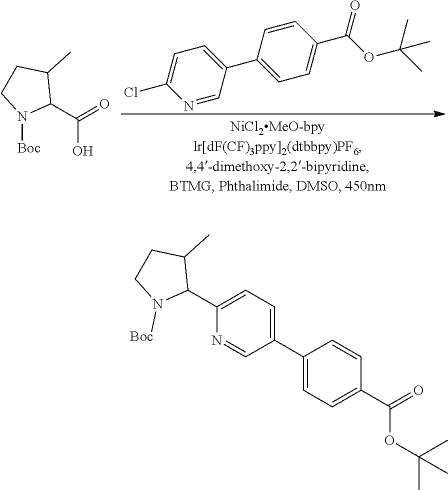

A solution of 1-(tert-butoxycarbonyl)-3-methylpyrrolidine-2-carboxylic acid (688 mg, 3.0 mmol), tert-butyl 4-(6-chloropyridin-3-yl)benzoate (580 mg, 2.00 mmol), Nickel(II) chloride ethylene glycol dimethyl ether complex (44.0 mg, 0.200 mmol), 4,4′-dimethoxy-2,2′-bipyridine (52 mg, 0.241 mmol), N″-tert-butyl-N,N,N′,N′-tetramethylguanidine (514 mg, 3.00 mmol), Ir[dF(CF₃)ppy]₂(dtbpy))PF₆ (45 mg, 40.0 μmol) and 2,3-dihydro-1H-isoindole-1,3-dione (295 mg, 2.0 mmol) in DMSO (12.0 mL) at room temperature was stirred under 450 nm blue LED irradiation for 24 hours under nitrogen atmosphere. Upon completion, the mixture was diluted with water and extracted with ethyl acetate (3×30 mL). The combined organic layers were washed with brine, dried over anhydrous Na₂SO₄, filtered, and concentrated under reduced pressure. The crude residue was purified by reversed-phase flash chromatography (column: C18 silica gel; mobile phase A: water (0.1% formic acid), mobile phase B: acetonitrile; gradient (B %): 0% to 100% gradient in 25 min; detector: 254 nm) to afford tert-butyl 2-(5-(4-(tert-butoxycarbonyl)phenyl)pyridin-2-yl)-3-methylpyrrolidine-1-carboxylate (150 mg, 17%) as a yellow oil. LCMS [M+H]⁺: 439.2

### Step 4: 4-(6-(3-methylpyrrolidin-2-yl)pyridin-3-yl)benzoic acid

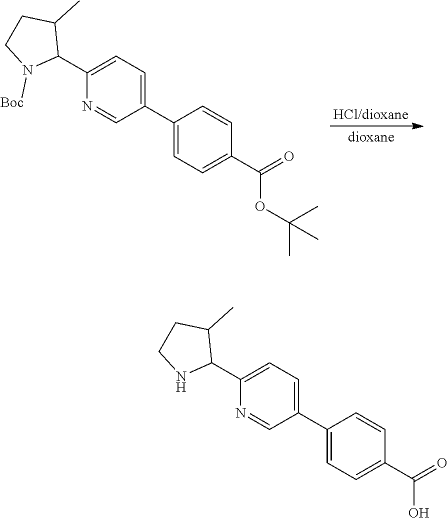 [SMILES: CC1CCN(C(=O)OC(C)(C)C)C1c1ccc(-c2ccc(C(=O)OC(C)(C)C)cc2)cn1 ; CC1CCNC1c1ccc(-c2ccc(C(=O)O)cc2)cn1]

To a mixture of tert-butyl 2-(5-(4-(tert-butoxycarbonyl)phenyl)pyridin-2-yl)-3-methylpyrrolidine-1-carboxylate (145 mg, 0.331 mmol) in 1,4-dioxane (1.0 mL) was added HCl (4M in 1,4-dioxane, 5.0 mL) dropwise at 0° C. The reaction mixture was warmed to room temperature and stirred overnight. Upon completion, the mixture was concentrated under reduced pressure to afford 4-(6-(3-methylpyrrolidin-2-yl)pyridin-3-yl)benzoic acid (130 mg, crude) as an off white solid, which was used in the next step without further purification. LCMS [M+H]⁺: 283.1

### Step 5: 4-(6-(1-((4-isopropylphenyl)carbamoyl)-3-methylpyrrolidin-2-yl)pyridin-3-yl)benzoic acid

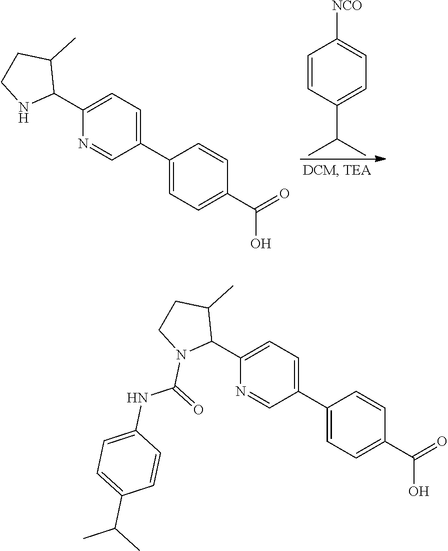

To a mixture of 4-(6-(3-methylpyrrolidin-2-yl)pyridin-3-yl)benzoic acid (120 mg, 0.426 mmol) and triethylamine (43.0 mg, 0.426 mmol) in DCM (5.0 mL) was added 1-isocyanato-4-isopropylbenzene (69.0 mg, 0.428 mmol) dropwise at 0° C. The resultant mixture was warmed to room temperature and stirred for 1 hour. Upon completion, the mixture was concentrated under reduced pressure. The crude residue was purified by reversed-phase flash chromatography (conditions: column: C18 silica gel; mobile phase A: water (0.1% formic acid), mobile phase B: acetonitrile; gradient (B %): 0% to 100% over 25 min; detector: 254 nm) to afford 4-(6-(1-((4-isopropylphenyl)carbamoyl)-3-methylpyrrolidin-2-yl)pyridin-3-yl)benzoic acid (100 mg, 53%) as a white solid. LCMS [M+H]⁺: 444.2

### Step 6: 4-(6-((2S,3S)-1-((4-isopropylphenyl)carbamoyl)-3-methylpyrrolidin-2-yl)pyridin-3-yl)benzoic acid & 4-(6-((2R,3R)-1-((4-isopropylphenyl)carbamoyl)-3-methylpyrrolidin-2-yl)pyridin-3-yl)benzoic acid

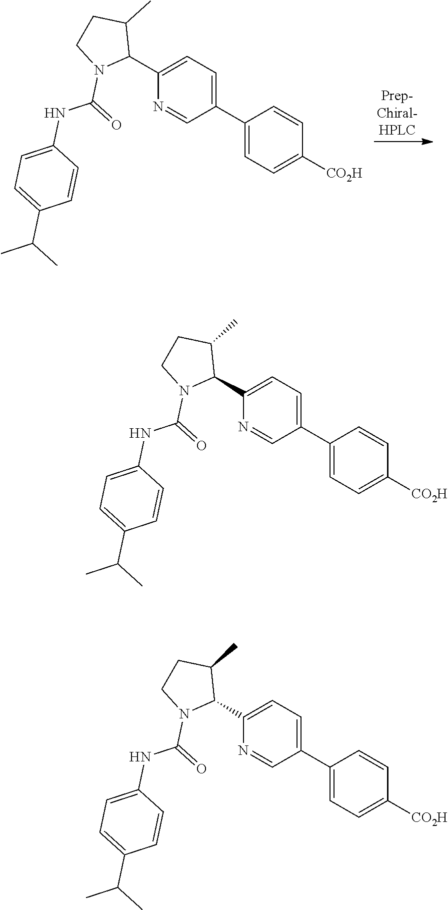 [SMILES: CC(C)c1ccc(NC(=O)N2CCC(C)C2c2ccc(-c3ccc(C(=O)O)cc3)cn2)cc1 ; CC(C)c1ccc(NC(=O)N2CC[C@H](C)[C@H]2c2ccc(-c3ccc(C(=O)O)cc3)cn2)cc1 ; CC(C)c1ccc(NC(=O)N2CC[C@@H](C)[C@@H]2c2ccc(-c3ccc(C(=O)O)cc3)cn2)cc1]

4-(6-(1-((4-isopropylphenyl)carbamoyl)-3-methylpyrrolidin-2-yl)pyridin-3-yl)benzoic acid (95 mg) was resolved by preparative chiral HPLC (conditions: column: CHIRALPAK IK 3*25 cm, 5 μm; mobile phase A: hexane (0.1% formic acid), mobile phase B: ethanol; Flow rate: 40 mL/min; gradient (B %): isocratic 30% B; detector: 220/254 nm; RT1 (min): 7.0; RT2 (min): 15.0) to afford first peak (Compound 124a; Method G, Peak 1, 1.77 min) 4-(6-((2S,3S)-1-((4-isopropylphenyl)carbamoyl)-3-methylpyrrolidin-2-yl)pyridin-3-yl)benzoic acid (45.2 mg, 48%) as a white solid, and second peak (Compound 124b; Method G, Peak 2, 2.49 min) 4-(6-((2R,3R)-1-((4-isopropylphenyl)carbamoyl)-3-methylpyrrolidin-2-yl)pyridin-3-yl)benzoic acid (43.8 mg, 46%) as a white solid.

| Cmpd # | Characterization Data |
|---|---|
| 124a | LCMS (ESI) [M + H]⁺: 444.2 |
| | ¹H NMR (400 MHz, DMSO-d₆) δ 8.87 (s, 1H), 8.18 (s, 1H), 8.08 (d, J = 7.6 Hz, 1H), 8.03 (d, J = 8.0 |
| | Hz, 2H), 7.82 (d, J = 8.0 Hz, 2H), 7.37-7.33 (m, 3H), 7.05 (d, J = 8.0 Hz, 2H), 4.64-4.62 (m, 1H), |
| | 3.75-3.73 (m, 2H), 2.81-2.74 (m, 1H), 2.33-2.28 (m, 1H), 2.12-2.08 (m, 1H), 1.68-1.63 (m, |
| | 1H), 1.14 (d, J = 7.2 Hz, 9H). |
| 124b | LCMS (ESI) [M + H]⁺: 444.2 |
| | ¹H NMR (400 MHz, DMSO-d₆) δ 8.87 (s, 1H), 8.18 (s, 1H), 8.08 (d, J = 10.4 Hz, 1H), 8.03 (d, J = 8.4 |
| | Hz, 2H), 7.83 (d, J = 8.0 Hz, 2H), 7.38-7.33 (m, 3H), 7.05 (d, J = 8.4 Hz, 2H), 4.64-4.62 (m, 1H), |
| | 3.77-3.72 (m, 2H), 2.80-2.77 (m, 1H), 2.32-2.29 (m, 1H), 2.11-2.08 (m, 1H), 1.68-1.64 (m, |
| | 1H), 1.14 (d, J = 7.2 Hz, 9H). |

### Examples 125-150 Compounds 125-150 in Table 1 were Synthesized Using the Following General Procedures

### General Synthetic Method 1:

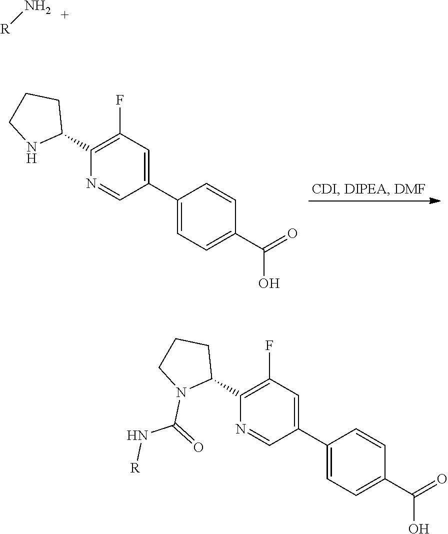

To a reaction vial was added amine building block (1.0 eq.) and CDI (1.0 equiv.), followed by anhydrous DMF (1.0 mL) and DIPEA (2.0 equiv.). The mixture was stirred at an ambient temperature for 1 hour. Then, (R)-4-(5-fluoro-6-(pyrrolidin-2-yl)pyridin-3-yl)benzoic acid (33.8 mg, 0.1 mmol, 1.0 equiv.) was added to the mixture while stirring. The mixture was stirred at ambient temperature for 10 hours. The progress of the reaction was analyzed by LCMS. Upon completion, the reaction mixture was filtered with a micropore filter and washed with DMF (1 mL). The filtration was purified directly by prep-HPLC (conditions: Table 2) to afford the desired product.

### General Synthetic Method 2:

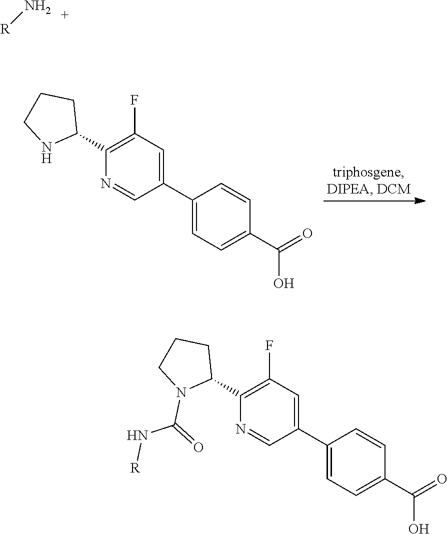

To a reaction vial was added amine building block (1.0 equiv.) and DIPEA (4.0 equiv.), followed by a solution of triphosgene (0.33 equiv.) in anhydrous DCM (1.0 mL). The mixture was cooled to 0˜5° C. and stirred for 2 hours. Then (R)-4-(5-fluoro-6-(pyrrolidin-2-yl)pyridin-3-yl)benzoic acid (33.8 mg, 0.1 mmol, 1.0 equiv.) was added to the mixture while stirring. The reaction mixture was warmed to ambient temperature and stirred for 10 hours. The progress of the reaction was analyzed by LCMS. Upon completion, the reaction mixture was filtered with a micropore filter and washed with DMF (1 mL). The filtration was purified directly by prep-HPLC (conditions: Table 2) to afford the desired product.

### General Synthetic Method 3:

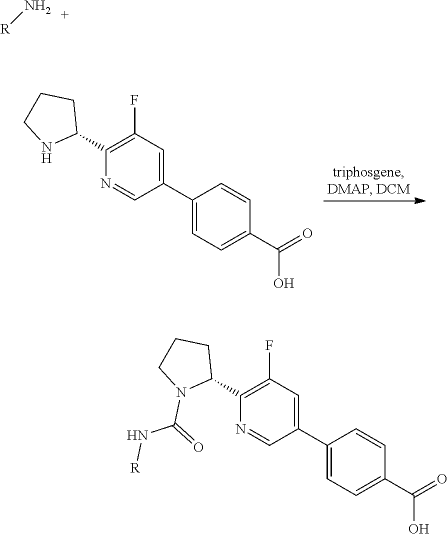

To a reaction vial was added amine building block (1.0 equiv.) and DMAP (4.0 equiv.), followed by a solution of triphosgene (0.33 equiv.) in anhydrous DCM (1.0 mL). The mixture was cooled to 0˜5° C. and stirred for 2 hours. Then (R)-4-(5-fluoro-6-(pyrrolidin-2-yl)pyridin-3-yl)benzoic acid (33.8 mg, 0.1 mmol, 1.0 equiv.) was added to the mixture while stirring. The reaction mixture was warmed to ambient temperature and stirred for 10 hours. The progress of the reaction was analyzed by LCMS. Upon completion, the reaction mixture was concentrated to dryness by blowing a steady stream of nitrogen into the vial. The crude residue was diluted with DMF (1.0 mL), filtered with a micropore filter and washed with DMF (1 mL). The filtrate was directly purified by prep-HPLC (conditions: Table 2) to afford the desired product.

TABLE 1
Compounds Prepared Using General Synthetic Methods 1-6

| Cmpd | | |
|---|---|---|
| # | Structure | Name |
| 125 | 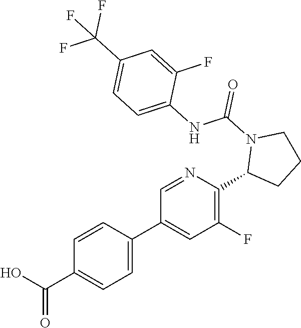 [SMILES: O=C(O)c1ccc(-c2cnc([C@H]3CCCN3C(=O)Nc3ccc(C(F)(F)F)cc3F)c(F)c2)cc1] | (R)-4-(5-fluoro-6-(1-((2-fluoro-4- (trifluoromethyl)phenyl) carbamoyl)pyrrolidin- 2-yl)pyridin-3-yl)benzoic acid |
| 126 | 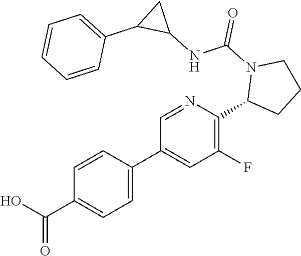 [SMILES: O=C(O)c1ccc(-c2cnc([C@H]3CCCN3C(=O)NC3CC3c3ccccc3)c(F)c2)cc1] | 4-(5-fluoro-6-((2R)-1-((2- phenylcyclopropyl) carbamoyl)pyrrolidin- 2-yl)pyridin-3-yl)benzoic acid |
| 128 | 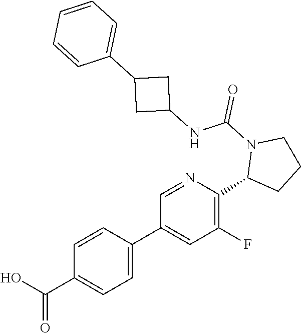 [SMILES: O=C(O)c1ccc(-c2cnc([C@H]3CCCN3C(=O)NC3CC(c4ccccc4)C3)c(F)c2)cc1] | (R)-4-(5-fluoro-6-(1-((3- phenylcyclobutyl) carbamoyl)pyrrolidin- 2-yl)pyridin-3-yl)benzoic acid |
| 129 | 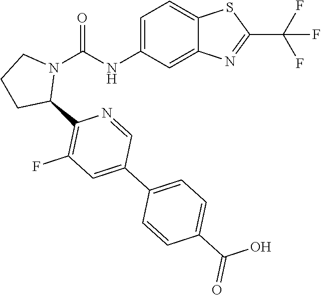 [SMILES: O=C(O)c1ccc(-c2cnc([C@H]3CCCN3C(=O)Nc3ccc4sc(C(F)(F)F)nc4c3)c(F)c2)cc1] | (R)-4-(5-fluoro-6-(1-((2- (trifluoromethyl)benzo[d]thiazol-5- yl)carbamoyl)pyrrolidin- 2-yl)pyridin-3- yl)benzoic acid |
| 130 | 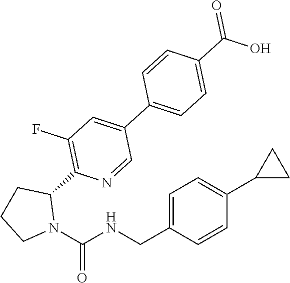 [SMILES: O=C(O)c1ccc(-c2cnc([C@H]3CCCN3C(=O)NCc3ccc(C4CC4)cc3)c(F)c2)cc1] | (R)-4-(6-(1-((4-cyclopropyl- benzyl)carbamoyl)pyrrolidin- 2-yl)-5-fluoropyridin- 3-yl)benzoic acid |
| 131 | 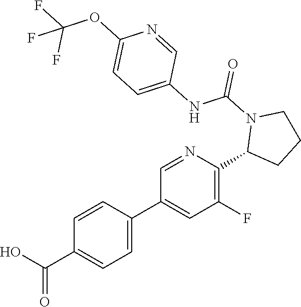 [SMILES: O=C(O)c1ccc(-c2cnc([C@H]3CCCN3C(=O)Nc3ccc(OC(F)(F)F)nc3)c(F)c2)cc1] | (R)-4-(5-fluoro-6-(1-((6- (trifluoromethoxy)pyridin-3- yl)carbamoyl)pyrrolidin- 2-yl)pyridin-3- yl)benzoic acid |
| 132 | 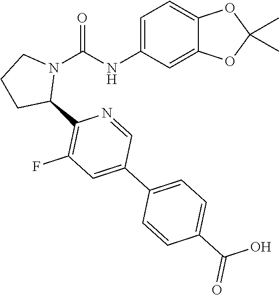 [SMILES: CC1(C)Oc2ccc(NC(=O)N3CCC[C@@H]3c3ncc(-c4ccc(C(=O)O)cc4)cc3F)cc2O1] | (R)-4-(6-(1-((2,2- dimethylbenzo[d][1,3]dioxol-5- yl)carbamoyl)pyrrolidin-2-yl)-5- fluoropyridin-3-yl)benzoic acid |
| 133 | 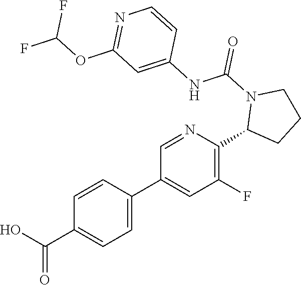 [SMILES: O=C(O)c1ccc(-c2cnc([C@H]3CCCN3C(=O)Nc3ccnc(OC(F)F)c3)c(F)c2)cc1] | (R)-4-(6-(1-((2- (difluoromethoxy)pyridin- 4-yl)carbamoyl)pyrrolidin-2-yl)-5- fluoropyridin-3-yl)benzoic acid |
| 137 | 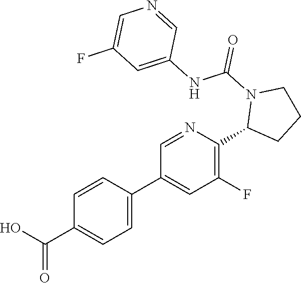 [SMILES: O=C(O)c1ccc(-c2cnc([C@H]3CCCN3C(=O)Nc3cncc(F)c3)c(F)c2)cc1] | (R)-4-(5-fluoro-6- (1-((5-fluoropyridin-3- yl)carbamoyl)pyrrolidin- 2-yl)pyridin-3- yl)benzoic acid |
| 138 | 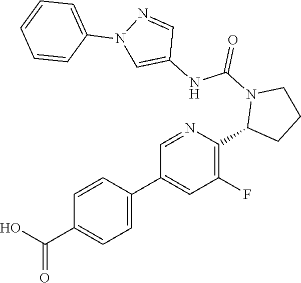 [SMILES: O=C(O)c1ccc(-c2cnc([C@H]3CCCN3C(=O)Nc3cnn(-c4ccccc4)c3)c(F)c2)cc1] | (R)-4-(5-fluoro-6-(1-((1-phenyl-1H- pyrazol-4-yl)carbamoyl)pyrrolidin-2- yl)pyridin-3-yl)benzoic acid |
| 139 | 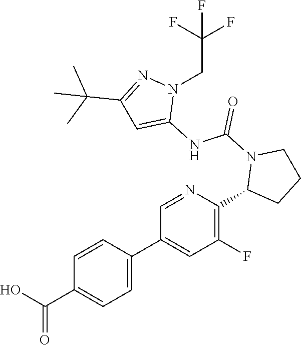 [SMILES: CC(C)(C)c1cc(NC(=O)N2CCC[C@@H]2c2ncc(-c3ccc(C(=O)O)cc3)cc2F)n(CC(F)(F)F)n1] | (R)-4-(6-(1-((3-(tert-butyl)-1-(2,2,2- trifluoroethyl)-1H-pyrazol-5- yl)carbamoyl)pyrrolidin-2-yl)-5- fluoropyridin-3-yl)benzoic acid |
| 140 | 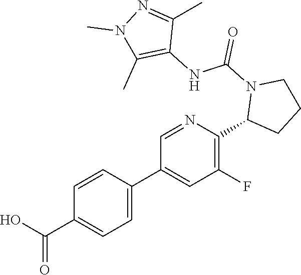 [SMILES: Cc1nn(C)c(C)c1NC(=O)N1CCC[C@@H]1c1ncc(-c2ccc(C(=O)O)cc2)cc1F] | (R)-4-(5-fluoro-6-(1- ((1,3,5-trimethyl-1H- pyrazol-4-yl)carbamoyl)pyrrolidin-2- yl)pyridin-3-yl)benzoic acid |
| 141 | 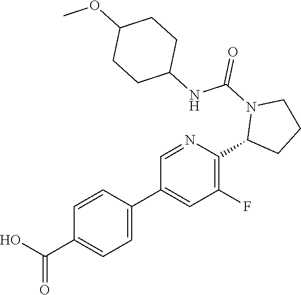 [SMILES: COC1CCC(NC(=O)N2CCC[C@@H]2c2ncc(-c3ccc(C(=O)O)cc3)cc2F)CC1] | (R)-4-(5-fluoro-6-(1-((4- methoxycyclohexyl) carbamoyl)pyrrolidin- 2-yl)pyridin-3-yl)benzoic acid |
| 142 | 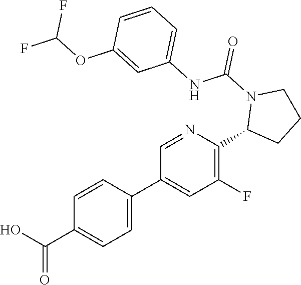 [SMILES: O=C(O)c1ccc(-c2cnc([C@H]3CCCN3C(=O)Nc3cccc(OC(F)F)c3)c(F)c2)cc1] | (R)-4-(6-(1-((3- (difluoromethoxy)phenyl)carbamoyl) pyrrolidin-2-yl)-5-fluoropyridin- 3-yl)benzoic acid |
| 143 | 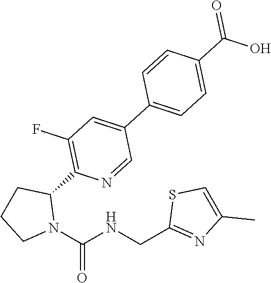 [SMILES: Cc1csc(CNC(=O)N2CCC[C@@H]2c2ncc(-c3ccc(C(=O)O)cc3)cc2F)n1] | (R)-4-(5-fluoro-6-(1- (((4-methylthiazol-2- yl)methyl)carbamoyl)pyrrolidin-2- yl)pyridin-3-yl)benzoic acid |
| 144 | 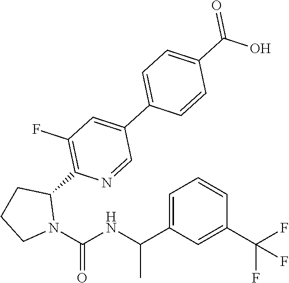 [SMILES: CC(NC(=O)N1CCC[C@@H]1c1ncc(-c2ccc(C(=O)O)cc2)cc1F)c1cccc(C(F)(F)F)c1] | 4-(5-fluoro-6-((2R)-1-((1-(3- (trifluoromethyl)phenyl) ethyl)carbamoyl)pyrrolidin- 2-yl)pyridin-3-yl)benzoic acid |
| 145 | 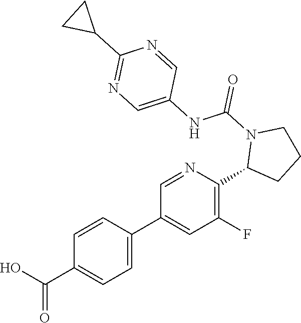 [SMILES: O=C(O)c1ccc(-c2cnc([C@H]3CCCN3C(=O)Nc3cnc(C4CC4)nc3)c(F)c2)cc1] | (R)-4-(6-(1-((2- cyclopropylpyrimidin-5- yl)carbamoyl)pyrrolidin-2-yl)-5- fluoropyridin-3-yl)benzoic acid |
| 147 | 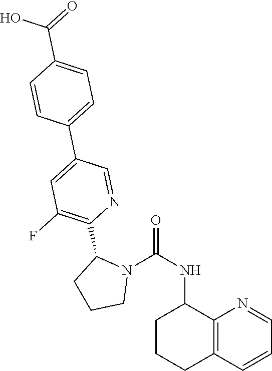 [SMILES: O=C(O)c1ccc(-c2cnc([C@H]3CCCN3C(=O)NC3CCCc4cccnc43)c(F)c2)cc1] | 4-(5-fluoro-6-((2R)-1-((5,6,7,8- tetrahydroquinolin-8- yl)carbamoyl)pyrrolidin- 2-yl)pyridin-3- yl)benzoic acid |
| 149 | 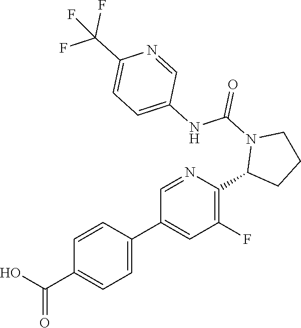 [SMILES: O=C(O)c1ccc(-c2cnc([C@H]3CCCN3C(=O)Nc3ccc(C(F)(F)F)nc3)c(F)c2)cc1] | (R)-4-(5-fluoro-6-(1-((6- (trifluoromethyl)pyridin-3- yl)carbamoyl)pyrrolidin- 2-yl)pyridin-3- yl)benzoic acid |
| 150 | 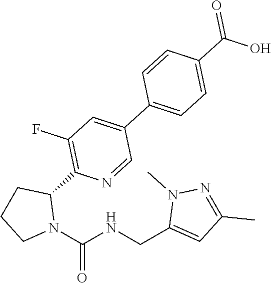 [SMILES: Cc1cc(CNC(=O)N2CCC[C@@H]2c2ncc(-c3ccc(C(=O)O)cc3)cc2F)n(C)n1] | (R)-4-(6-(1-(((1,3- dimethyl-1H-pyrazol- 5-yl)methyl)carbamoyl) pyrrolidin-2-yl)- 5-fluoropyridin-3-yl)benzoic acid |
| 244 | 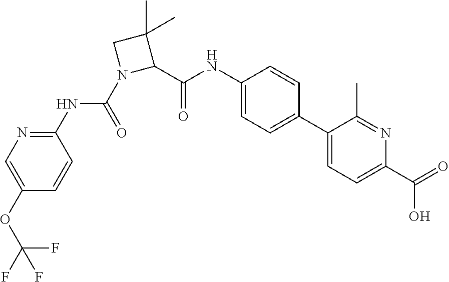 [SMILES: Cc1nc(C(=O)O)ccc1-c1ccc(NC(=O)C2N(C(=O)Nc3ccc(OC(F)(F)F)cn3)CC2(C)C)cc1] | 5-(4-(3,3-dimethyl-1-((5- (trifluoromethoxy)pyridin-2- yl)carbamoyl)azetidine-2- carboxamido)phenyl)- 6-methylpicolinic acid |
| 245 | 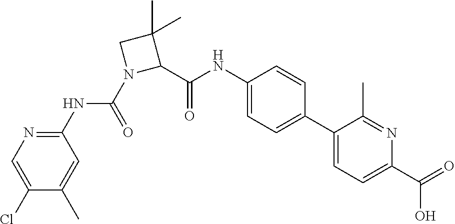 [SMILES: Cc1cc(NC(=O)N2CC(C)(C)C2C(=O)Nc2ccc(-c3ccc(C(=O)O)nc3C)cc2)ncc1Cl] | 5-(4-(1-((5-chloro-4-methylpyridin-2- yl)carbamoyl)-3,3- dimethylazetidine-2- carboxamido)phenyl)- 6-methylpicolinic acid |
| 246 | 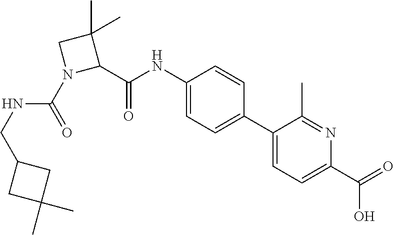 [SMILES: Cc1nc(C(=O)O)ccc1-c1ccc(NC(=O)C2N(C(=O)NCC3CC(C)(C)C3)CC2(C)C)cc1] | 5-(4-(1-(((3,3- dimethylcyclobutyl) methyl)carbamoyl)- 3,3-dimethylazetidine-2- carboxamido)phenyl)- 6-methylpicolinic acid |
| 250 | 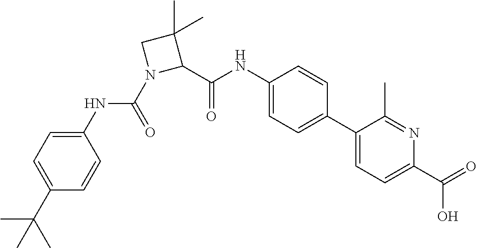 [SMILES: Cc1nc(C(=O)O)ccc1-c1ccc(NC(=O)C2N(C(=O)Nc3ccc(C(C)(C)C)cc3)CC2(C)C)cc1] | 5-(4-(1-((4-(tert- butyl)phenyl)carbamoyl)-3,3- dimethylazetidine-2- carboxamido)phenyl)- 6-methylpicolinic acid |
| 251 | 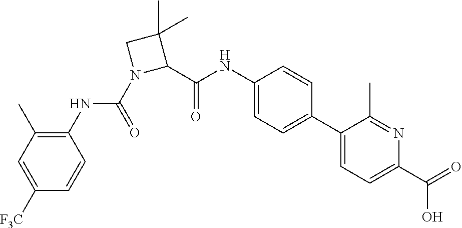 [SMILES: Cc1cc(C(F)(F)F)ccc1NC(=O)N1CC(C)(C)C1C(=O)Nc1ccc(-c2ccc(C(=O)O)nc2C)cc1] | 5-(4-(3,3-dimethyl-1-((2-methyl-4- (trifluoromethyl)phenyl)carbamoyl) azetidine-2-carboxamido)phenyl)-6- methylpicolinic acid |
| 252 | 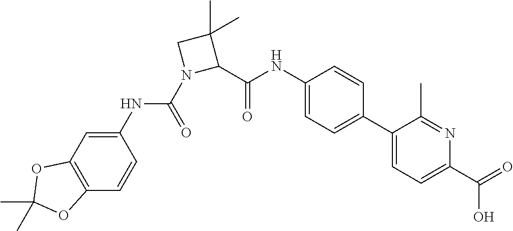 [SMILES: Cc1nc(C(=O)O)ccc1-c1ccc(NC(=O)C2N(C(=O)Nc3ccc4c(c3)OC(C)(C)O4)CC2(C)C)cc1] | 5-(4-(1-((2,2- dimethylbenzo[d][1,3]dioxol-5- yl)carbamoyl)-3,3- dimethylazetidine-2- carboxamido)phenyl)- 6-methylpicolinic acid |
| 254 | 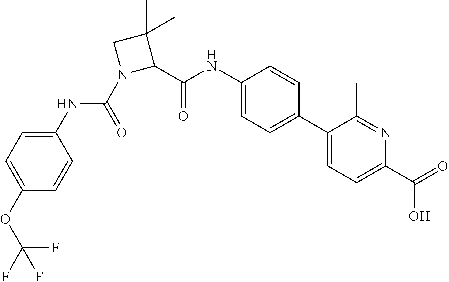 [SMILES: Cc1nc(C(=O)O)ccc1-c1ccc(NC(=O)C2N(C(=O)Nc3ccc(OC(F)(F)F)cc3)CC2(C)C)cc1] | 5-(4-(3,3-dimethyl-1-((4- (trifluoromethoxy)phenyl)carbamoyl) azetidine-2-carboxamido)phenyl)-6- methylpicolinic acid |
| 255 | 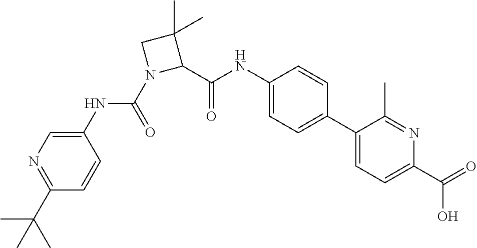 [SMILES: Cc1nc(C(=O)O)ccc1-c1ccc(NC(=O)C2N(C(=O)Nc3ccc(C(C)(C)C)nc3)CC2(C)C)cc1] | 5-(4-(1-((6-(tert-butyl)pyridin-3- yl)carbamoyl)-3,3- dimethylazetidine-2- carboxamido)phenyl)- 6-methylpicolinic acid |
| 256 | 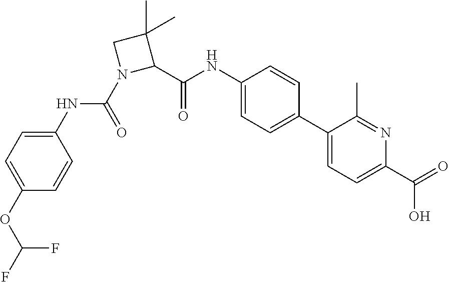 [SMILES: Cc1nc(C(=O)O)ccc1-c1ccc(NC(=O)C2N(C(=O)Nc3ccc(OC(F)F)cc3)CC2(C)C)cc1] | 5-(4-(1-((4-(difluoromethoxy) phenyl)carbamoyl)-3,3- dimethylazetidine-2- carboxamido)phenyl)- 6-methylpicolinic acid |
| 258 | 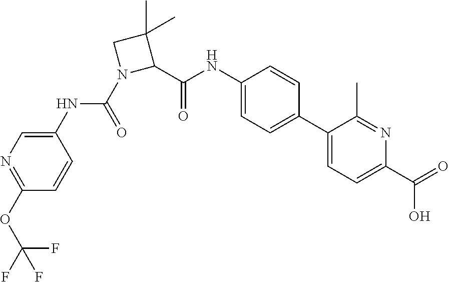 [SMILES: Cc1nc(C(=O)O)ccc1-c1ccc(NC(=O)C2N(C(=O)Nc3ccc(OC(F)(F)F)nc3)CC2(C)C)cc1] | 5-(4-(3,3-dimethyl-1-((6- (trifluoromethoxy)pyridin-3- yl)carbamoyl)azetidine-2- carboxamido)phenyl)- 6-methylpicolinic acid |
| 259 | 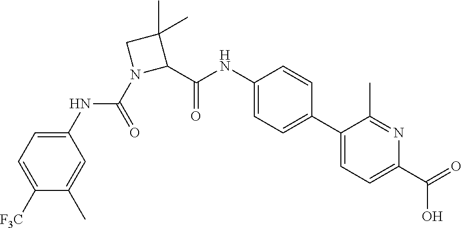 [SMILES: Cc1cc(NC(=O)N2CC(C)(C)C2C(=O)Nc2ccc(-c3ccc(C(=O)O)nc3C)cc2)ccc1C(F)(F)F] | 5-(4-(3,3-dimethyl-1-((3-methyl-4- (trifluoromethyl)phenyl)carbamoyl) azetidine-2-carboxamido)phenyl)-6- methylpicolinic acid |
| 260 | 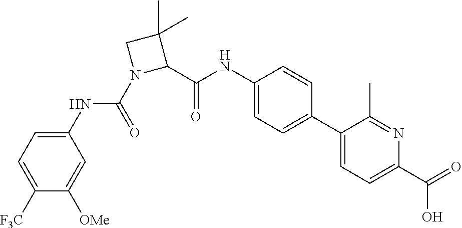 [SMILES: COc1cc(NC(=O)N2CC(C)(C)C2C(=O)Nc2ccc(-c3ccc(C(=O)O)nc3C)cc2)ccc1C(F)(F)F] | 5-(4-(1-((3-methoxy-4- (trifluoromethyl)phenyl)carbamoyl)- 3,3-dimethylazetidine-2- carboxamido)phenyl)- 6-methylpicolinic acid |
| 262 | 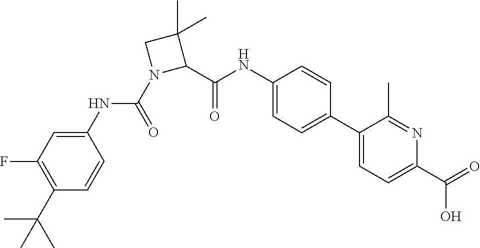 [SMILES: Cc1nc(C(=O)O)ccc1-c1ccc(NC(=O)C2N(C(=O)Nc3ccc(C(C)(C)C)c(F)c3)CC2(C)C)cc1] | 5-(4-(1-((4-(tert-butyl)-3- fluorophenyl)carbamoyl)-3,3- dimethylazetidine-2- carboxamido)phenyl)- 6-methylpicolinic acid |
| 263 | 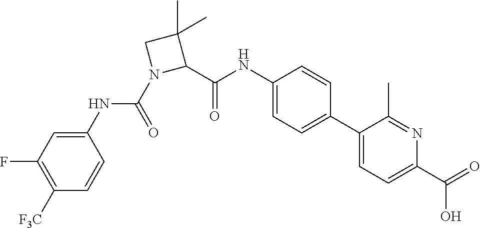 [SMILES: Cc1nc(C(=O)O)ccc1-c1ccc(NC(=O)C2N(C(=O)Nc3ccc(C(F)(F)F)c(F)c3)CC2(C)C)cc1] | 5-(4-(1-((3-fluoro-4- (trifluoromethyl)phenyl)carbamoyl)- 3,3-dimethylazetidine-2- carboxamido)phenyl)- 6-methylpicolinic acid |
| 264 | 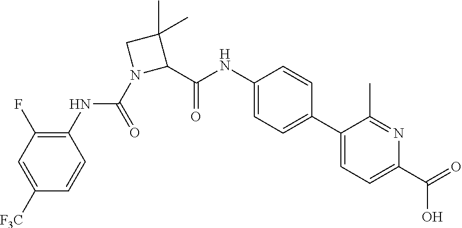 [SMILES: Cc1nc(C(=O)O)ccc1-c1ccc(NC(=O)C2N(C(=O)Nc3ccc(C(F)(F)F)cc3F)CC2(C)C)cc1] | 5-(4-(1-((2-fluoro-4- (trifluoromethyl)phenyl)carbamoyl)- 3,3-dimethylazetidine-2- carboxamido)phenyl)- 6-methylpicolinic acid |
| 265 | 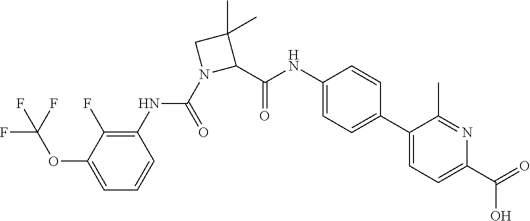 [SMILES: Cc1nc(C(=O)O)ccc1-c1ccc(NC(=O)C2N(C(=O)Nc3cccc(OC(F)(F)F)c3F)CC2(C)C)cc1] | 5-(4-(1-((2-fluoro-3- (trifluoromethoxy)phenyl)carbamoyl)- 3,3-dimethylazetidine-2- carboxamido)phenyl)- 6-methylpicolinic acid |
| 266 | 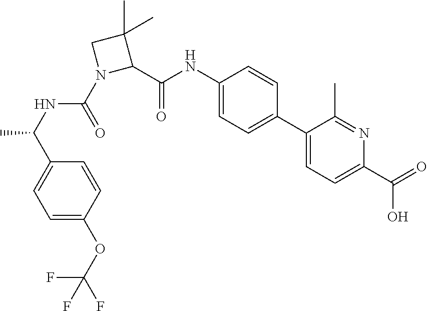 [SMILES: Cc1nc(C(=O)O)ccc1-c1ccc(NC(=O)C2N(C(=O)N[C@@H](C)c3ccc(OC(F)(F)F)cc3)CC2(C)C)cc1] | 5-(4-(3,3-dimethyl-1-(((S)-1-(4- (trifluoromethoxy)phenyl) ethyl)carbamoyl) azetidine-2-carboxamido)phenyl)-6- methylpicolinic acid |
| 267 | 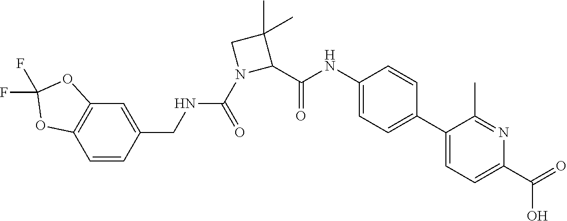 [SMILES: Cc1nc(C(=O)O)ccc1-c1ccc(NC(=O)C2N(C(=O)NCc3ccc4c(c3)OC(F)(F)O4)CC2(C)C)cc1] | 5-(4-(1-(((2,2- difluorobenzo[d][1,3]dioxol-5- yl)methyl)carbamoyl)-3,3- dimethylazetidine-2- carboxamido)phenyl)- 6-methylpicolinic acid |
| 269 | 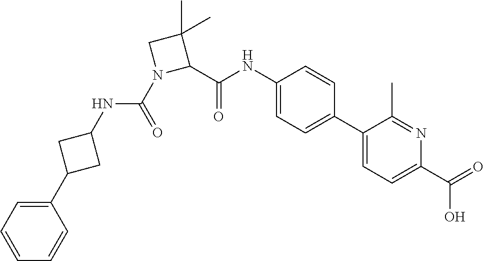 [SMILES: Cc1nc(C(=O)O)ccc1-c1ccc(NC(=O)C2N(C(=O)NC3CC(c4ccccc4)C3)CC2(C)C)cc1] | 5-(4-(3,3-dimethyl-1-((3- phenylcyclobutyl)carbamoyl) azetidine-2-carboxamido) phenyl)-6-methylpicolinic acid |
| 270 | 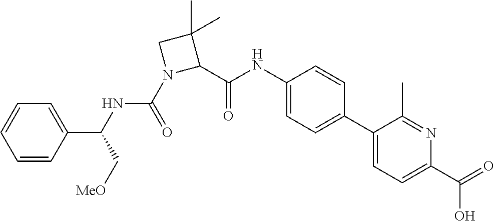 [SMILES: COC[C@@H](NC(=O)N1CC(C)(C)C1C(=O)Nc1ccc(-c2ccc(C(=O)O)nc2C)cc1)c1ccccc1] | 5-(4-(1-(((S)-2-methoxy-1- phenylethyl)carbamoyl)-3,3- dimethylazetidine-2- carboxamido)phenyl)- 6-methylpicolinic acid |
| 271 | 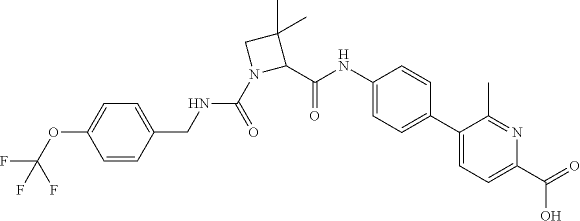 [SMILES: Cc1nc(C(=O)O)ccc1-c1ccc(NC(=O)C2N(C(=O)NCc3ccc(OC(F)(F)F)cc3)CC2(C)C)cc1] | 5-(4-(3,3-dimethyl-1-((4- (trifluoromethoxy)benzyl)carbamoyl) azetidine-2-carboxamido)phenyl)-6- methylpicolinic acid |
| 272 | 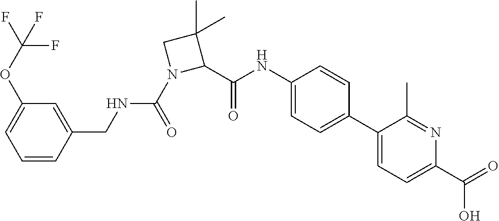 [SMILES: Cc1nc(C(=O)O)ccc1-c1ccc(NC(=O)C2N(C(=O)NCc3cccc(OC(F)(F)F)c3)CC2(C)C)cc1] | 5-(4-(3,3-dimethyl-1-((3- (trifluoromethoxy)benzyl)carbamoyl) azetidine-2-carboxamido)phenyl)-6- methylpicolinic acid |
| 273 | 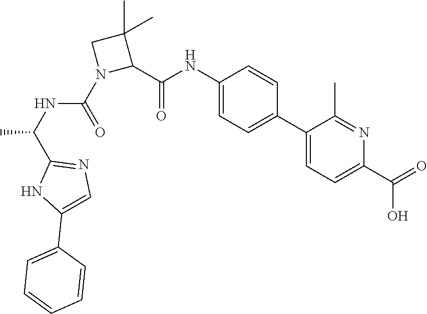 [SMILES: Cc1nc(C(=O)O)ccc1-c1ccc(NC(=O)C2N(C(=O)N[C@@H](C)c3ncc(-c4ccccc4)[nH]3)CC2(C)C)cc1] | 5-(4-(3,3-dimethyl-1-(((S)-1-(5- phenyl-1H-imidazol-2- yl)ethyl)carbamoyl)azetidine-2- carboxamido)phenyl)- 6-methylpicolinic acid |
| 274 | 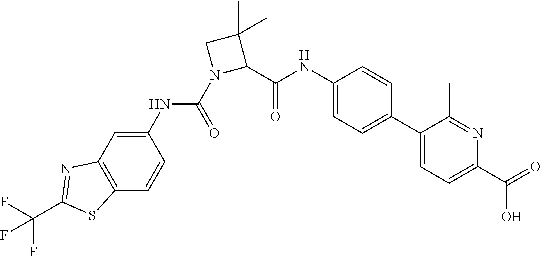 [SMILES: Cc1nc(C(=O)O)ccc1-c1ccc(NC(=O)C2N(C(=O)Nc3ccc4sc(C(F)(F)F)nc4c3)CC2(C)C)cc1] | 5-(4-(3,3-dimethyl-1-((2- (trifluoromethyl)benzo[d]thiazol-5- yl)carbamoyl)azetidine-2- carboxamido)phenyl)- 6-methylpicolinic acid |
| 275 | 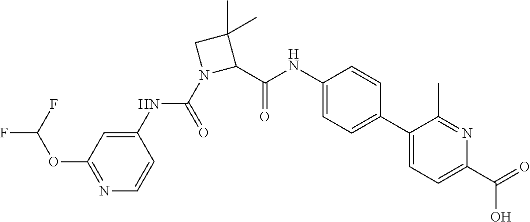 [SMILES: Cc1nc(C(=O)O)ccc1-c1ccc(NC(=O)C2N(C(=O)Nc3ccnc(OC(F)F)c3)CC2(C)C)cc1] | 5-(4-(1-((2-(difluoro- methoxy)pyridin-4- yl)carbamoyl)-3,3- dimethylazetidine-2- carboxamido)phenyl)- 6-methylpicolinic acid |
| 277 | 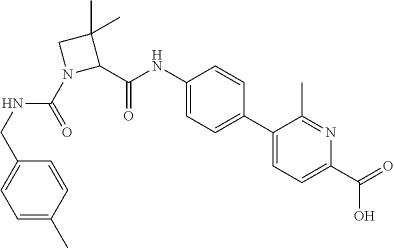 [SMILES: Cc1ccc(CNC(=O)N2CC(C)(C)C2C(=O)Nc2ccc(-c3ccc(C(=O)O)nc3C)cc2)cc1] | 5-(4-(3,3-dimethyl-1-((4- methylbenzyl)carbamoyl) azetidine-2- carboxamido)phenyl)- 6-methylpicolinic acid |
| 278 | 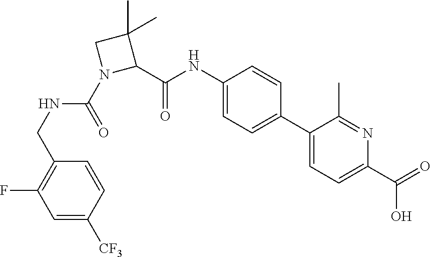 [SMILES: Cc1nc(C(=O)O)ccc1-c1ccc(NC(=O)C2N(C(=O)NCc3ccc(C(F)(F)F)cc3F)CC2(C)C)cc1] | 5-(4-(1-((2-fluoro-4- (trifluoromethyl)benzyl) carbamoyl)-3,3- dimethylazetidine-2- carboxamido)phenyl)- 6-methylpicolinic acid |
| 279 | 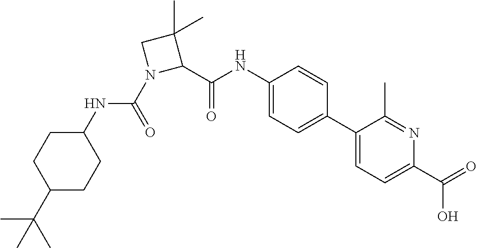 [SMILES: Cc1nc(C(=O)O)ccc1-c1ccc(NC(=O)C2N(C(=O)NC3CCC(C(C)(C)C)CC3)CC2(C)C)cc1] | 5-(4-(1-((4-(tert- butyl)cyclohexyl)carbamoyl)-3,3- dimethylazetidine-2- carboxamido)phenyl)- 6-methylpicolinic acid |
| 280 | 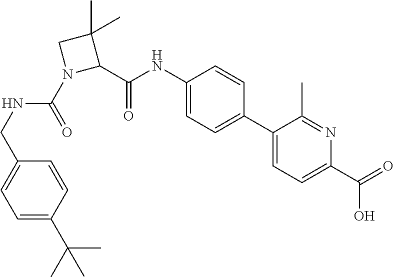 [SMILES: Cc1nc(C(=O)O)ccc1-c1ccc(NC(=O)C2N(C(=O)NCc3ccc(C(C)(C)C)cc3)CC2(C)C)cc1] | 5-(4-(1-((4-(tert-butyl) benzyl)carbamoyl)- 3,3-dimethylazetidine-2- carboxamido)phenyl)- 6-methylpicolinic acid |
| 281 | 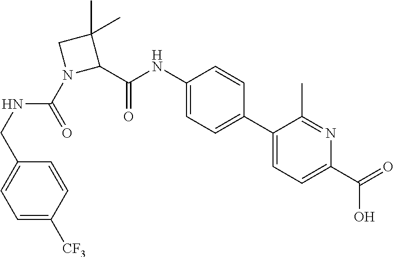 [SMILES: Cc1nc(C(=O)O)ccc1-c1ccc(NC(=O)C2N(C(=O)NCc3ccc(C(F)(F)F)cc3)CC2(C)C)cc1] | 5-(4-(3,3-dimethyl-1-((4- (trifluoromethyl)benzyl)carbamoyl) azetidine-2-carboxamido)phenyl)-6- methylpicolinic acid |
| 282 | 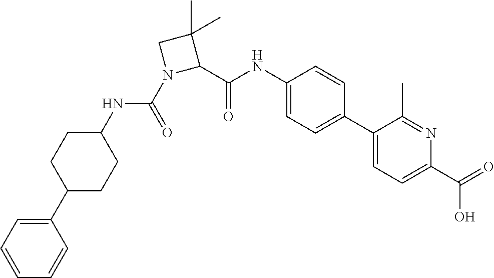 [SMILES: Cc1nc(C(=O)O)ccc1-c1ccc(NC(=O)C2N(C(=O)NC3CCC(c4ccccc4)CC3)CC2(C)C)cc1] | 5-(4-(3,3-dimethyl-1-((4- phenylcyclohexyl)carbamoyl) azetidine-2-carboxamido) phenyl)-6-methylpicolinic acid |
| 284 | 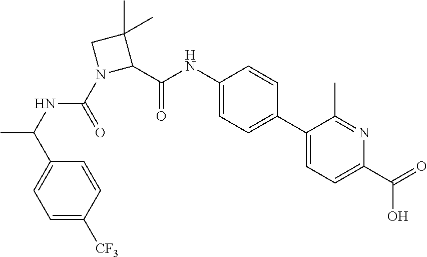 [SMILES: Cc1nc(C(=O)O)ccc1-c1ccc(NC(=O)C2N(C(=O)NC(C)c3ccc(C(F)(F)F)cc3)CC2(C)C)cc1] | 5-(4-(3,3-dimethyl-1-((1-(4- (trifluoromethyl)phenyl) ethyl)carbamoyl)azetidine-2- carboxamido)phenyl)-6- methylpicolinic acid |
| 285 | 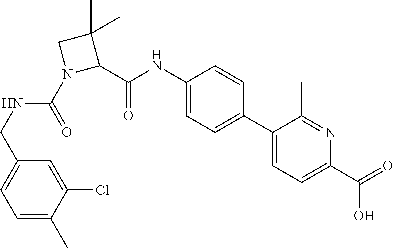 [SMILES: Cc1ccc(CNC(=O)N2CC(C)(C)C2C(=O)Nc2ccc(-c3ccc(C(=O)O)nc3C)cc2)cc1Cl] | 5-(4-(1-((3-chloro-4- methylbenzyl)carbamoyl)-3,3- dimethylazetidine-2- carboxamido)phenyl)- 6-methylpicolinic acid |

TABLE 2
Synthetic Methods and Characterization Data for Compounds 125-150

| | | Amine | | |
|---|---|---|---|---|
| | | Building | | |
| | | Block | | |
| Cmpd | | Structure | Prep-HPLC Purification | ¹H NMR; Mass Spectrum m/z [M + H]⁺ |
| # | Method | and Name | Conditions | (unless otherwise noted) |
| 125 | 2 | 2-fluoro-4- | Column: Phenomenex-Luna-C18- | ¹H NMR (400 MHz, DMSO-d₆) δ 13.08 |
| | | (trifluoro- | 10 μm-25*250 mm, Mobile | (bs, 1H), 8.76 (s, 1H), 8.24 (bs, 1H), |
| | | methyl)aniline | phase: A: 0.1% FA/H2O B: ACN, | 8.09 (dd, J = 1.4, 11.6 Hz, 1H), 8.03 (d, |
| | | | Gradient (B %): 52% to 82% in 12 | J = 8.4 Hz, 2H), 7.90 (d, J = 8.4 Hz, 2H), |
| | | | min, Flow rate: 30 mL/min, UV | 7.84 (t, J = 8.2 Hz, 1H), 7.61 (d, J = 10.1 |
| | | | wavelength: 220 nm | Hz, 1H), 7.44 (d, J = 8.8 Hz, 1H), 5.37 |
| | | | | (bs, 1H), 3.84-3.76 (m, 1H), 3.74-3.64 |
| | | | | (m, 1H), 2.45-2.31 (m, 1H), 2.16-1.88 |
| | | | | (m, 3H); 492.0 |
| 126 | 1 | 2- | Column: Phenomenex-Luna-C18- | ¹H NMR (400 MHz, Methanol-d₄) δ |
| | | phenylcyclo- | 10 μm-25*250 mm, Mobile phase: | 8.66-8.60 (m, 1H), 8.13 (d, J = 8.0 Hz, |
| | | propan-1-amine | A: 0.1% FA/H2O B: ACN, | 2H), 7.92-7.83 (m, 1H), 7.78 (dd, J = |
| | | | Gradient (B %): 42% to 72% in 12 | 8.4, 2.2 Hz, 2H), 7.23-7.14 (m, 2H), |
| | | | min, Flow rate: 30 mL/min, UV | 7.13-7.03 (m, 3H), 5.43-5.30 (m, |
| | | | wavelength: 220 nm | 1H), 3.75-3.61 (m, 1H), 3.49 (q, J = |
| | | | | 7.6 Hz, 1H), 2.74-2.63 (m, 1H), 2.48- |
| | | | | 2.32 (m, 1H), 2.13-1.92 (m, 4H), 1.19- |
| | | | | 1.02 (m, 2H); 446.0 |
| 128 | 1 | 3- | Column: Agela-Durashell-C18-10 | ¹H NMR (400 MHz, Methanol-d₄) δ |
| | | phenylcyclo- | μm-25*250 mm, Mobile phase: | 8.62 (d, J = 1.5 Hz, 1H), 8.13 (d, J = 8.4 |
| | | butan-1-amine | A: 0.1% FA/H2O B: ACN, | Hz, 2H), 7.88 (dd, J = 11.2, 1.8 Hz, 1H), |
| | | | Gradient (B %): 46% to 76% in 12 | 7.77 (d, J = 8.4 Hz, 2H), 7.28-7.19 (m, |
| | | | min, Flow rate: 30, UV | 4H), 7.16-7.06 (m, 1H), 5.35 (dd, J = |
| | | | wavelength: 220 nm | 8.7, 3.7 Hz, 1H), 4.34-4.08 (m, 1H), |
| | | | | 3.76-3.65 (m, 1H), 3.59-3.45 (m, |
| | | | | 1H), 3.04-3.02 (m, 1H), 2.71-2.59 |
| | | | | (m, 2H), 2.50-2.34 (m, 2H), 2.19- |
| | | | | 1.96 (m, 4H); 458.3 |
| 129 | 2 | 2- | Column: Agela-Durashell-C18-10 | ¹H NMR (400 MHz, Methanol-d₄) δ |
| | | (trifluoro- | μm-25*250 mm, Mobile phase: | 8.65 (s, 1H), 8.26 (d, J = 2.0 Hz, 1H), |
| | | methyl)benzo[d]thiazol- | A: 0.1% FA/H2O B: ACN, | 8.12 (d, J = 8.4 Hz, 2H), 7.97 (d, J = 8.9 |
| | | 5-amine | Gradient (B %): 50% to 80% in 12 | Hz, 1H), 7.90 (dd, J = 11.2, 1.9 Hz, 1H), |
| | | | min, Flow rate: 30 mL/min, UV | 7.77 (d, J = 8.4 Hz, 2H), 7.69 (dd, J = |
| | | | wavelength: 220 nm | 8.9, 2.1 Hz, 1H), 5.51 (dd, J = 8.3, 4.2 |
| | | | | Hz, 1H), 3.99-3.86 (m, 1H), 3.84- |
| | | | | 3.69 (m, 1H), 2.60-2.46 (m, 1H), 2.26- |
| | | | | 1.98 (m, 3H); 531.3 |
| 130 | 1 | (4- | Column: Agela-Durashell-C18-10 | ¹H NMR (400 MHz, Methanol-d₄) δ |
| | | cyclopro- | μm-25*250 mm, Mobile phase: | 8.61 (s, 1H), 8.14 (d, J = 8.4 Hz, 2H), |
| | | pylphenyl)methanamine | A: 0.1% FA/H2O B: ACN, | 7.86 (dd, J = 11.2, 1.9 Hz, 1H), 7.78 (d, |
| | | | Gradient (B %): 45% to 75% in 12 | J = 8.3 Hz, 2H), 7.08 (d, J = 7.9 Hz, 2H), |
| | | | min, Flow rate: 30 mL/min, UV | 6.94 (d, J = 8.1 Hz, 2H), 5.35 (dd, J = |
| | | | wavelength: 220 nm | 6.8, 3.3 Hz, 1H), 4.31 (d, J = 15.2 Hz, |
| | | | | 1H), 4.17 (d, J = 15.2 Hz, 1H), 3.78- |
| | | | | 3.65 (m, 1H), 3.55 (q, J = 8.0, 7.6 Hz, |
| | | | | 1H), 2.48-2.34 (m, 1H), 2.13-1.94 |
| | | | | (m, 3H), 1.89-1.76 (m, 1H), 0.94- |
| | | | | 0.82 (m, 2H), 0.62-0.52 (m, 2H); 458.3 |
| 131 | 2 | 6- | Column: Agela-Durashell-C18-10 | ¹H NMR (400 MHz, Methanol-d₄) δ |
| | | (trifluoromethoxy)pyridin- | μm-25*250 mm, Mobile phase: | 8.64 (s, 1H), 8.32 (d, J = 2.8 Hz, 1H), |
| | | 3-amine | A: 0.1% FA/H2O B: ACN, | 8.13 (d, J = 8.4 Hz, 2H), 8.00 (dd, J = |
| | | | Gradient (B %): 44% to 74% in 12 | 8.9, 2.8 Hz, 1H), 7.90 (dd, J = 11.2, 1.9 |
| | | | min, Flow rate: 30 mL/min, UV | Hz, 1H), 7.78 (d, J = 8.5 Hz, 2H), 7.06 |
| | | | wavelength: 220 nm | (d, J = 8.8 Hz, 1H), 5.52-5.42 (m, 1H), |
| | | | | 3.94-3.83 (m, 1H), 3.72 (dt, J = 9.3, |
| | | | | 7.1 Hz, 1H), 2.56-2.39 (m, 1H), 2.26- |
| | | | | 1.99 (m, 3H); 489.3 |
| 132 | 2 | 2,2- | Column: Agela-Durashell-C18-10 | ¹H NMR (400 MHz, Methanol-d₄) δ |
| | | dimethyl- | μm-25*250 mm, Mobile phase: | 8.64 (s, 1H), 8.13 (d, J = 8.4 Hz, 2H), |
| | | benzo[d][1,3]dioxol- | A: 0.1% FA/H2O B: ACN, | 7.88 (dd, J = 11.2, 1.9 Hz, 1H), 7.78 (d, |
| | | 5-amine | Gradient (B %): 44% to 74% in 12 | J = 8.4 Hz, 2H), 6.81 (d, J = 2.1 Hz, 1H), |
| | | | min, Flow rate: 30 mL/min, UV | 6.65 (dd, J = 8.3, 2.1 Hz, 1H), 6.56 (d, |
| | | | wavelength: 220 nm | J = 8.3 Hz, 1H), 5.48-5.37 (m, 1H), 3.90- |
| | | | | 3.76 (m, 1H), 3.66 (dt, J = 9.2, 7.1 Hz, |
| | | | | 1H), 2.49-2.36 (m, 1H), 2.26-2.13 |
| | | | | (m, 1H), 2.13-2.05 (m, 1H), 2.05- |
| | | | | 1.98 (m, 1H), 1.59 (s, 6H); 478.1 |
| 133 | 3 | 2- | Column: Agela-Durashell-C18-10 | ¹H NMR (400 MHz, DMSO-d₆) δ 13.09 |
| | | (difluoro- | μm-25*250 mm, Mobile phase: | (bs, 1H), 8.95 (s, 1H), 8.74 (s, 1H), 8.10 |
| | | methoxy)pyridin- | A: 0.1% FA/H2O B: ACN, | (dd, J = 1.8, 11.5 Hz, 1H), 8.03 (d, J = |
| | | 4-amine | Gradient (B %): 40% to 70% in 12 | 8.5 Hz, 2H), 7.96 (d, J = 5.9 Hz, 1H), |
| | | | min, Flow rate: 30 mL/min, UV | 7.90 (d, J = 8.4 Hz, 2H), 7.81-7.42 (m, |
| | | | wavelength: 220 nm | 1H), 7.36-7.29 (m, 1H), 7.19 (d, J = 1.6 |
| | | | | Hz, 1H), 5.36 (bs, 1H), 3.84-3.73 (m, |
| | | | | 1H), 3.73-3.62 (m, 1H), 2.48-2.27 (m, |
| | | | | 1H), 2.16-1.88 (m, 3H); 473.0 |
| 137 | 3 | 5- | Column: Welch-Xtimate-C18-7 | ¹H NMR (400 MHz, Methanol-d₄) δ |
| | | fluoropyridin- | μm-30*250 mm, Mobile phase: | 8.64 (d, J = 1.5 Hz, 1H), 8.43 (s, 1H), |
| | | 3-amine | A: 0.1% FA/H2O B: ACN, | 8.13 (d, J = 8.5 Hz, 2H), 8.04 (d, J = 2.5 |
| | | | Gradient (B %): 20% to 60% in 13 | Hz, 1H), 7.95-7.80 (m, 2H), 7.78 (d, J = |
| | | | min, Flow rate: 35 mL/min, UV | 8.4 Hz, 2H), 5.52-5.41 (m, 1H), 3.94- |
| | | | wavelength: 254 nm | 3.82 (m, 1H), 3.73 (dt, J = 9.3, 7.1 Hz, |
| | | | | 1H), 2.54-2.40 (m, 1H), 2.27-1.97 |
| | | | | (m, 3H); 425.1 |
| 138 | 3 | 1-phenyl-1H- | Column: Puningtech-Pntulips- | ¹H NMR (400 MHz, Methanol-d₄) δ |
| | | pyrazol-4- | C18-10 μm-30*250 mm, Mobile | 8.64 (s, 1H), 8.17 (s, 1H), 8.13 (d, J = |
| | | amine | phase: A: 0.1% FA/H2O B: ACN, | 8.0 Hz, 2H), 7.91 (d, J = 11.1 Hz, 1H), |
| | | | Gradient (B %): 30% to 70% in 15 | 7.78 (d, J = 6.8 Hz, 2H), 7.67 (s, 1H), |
| | | | min, Flow rate: 35 mL/min, UV | 7.62 (d, J = 8.6 Hz, 2H), 7.43 (t, J = 7.7 |
| | | | wavelength: 220 nm | Hz, 2H), 7.26 (t, J = 7.4 Hz, 1H), 5.46 |
| | | | | (dd, J = 8.2, 4.0 Hz, 1H), 3.90-3.79 (m, |
| | | | | 1H), 3.68 (q, J = 7.7 Hz, 1H), 2.54- |
| | | | | 2.39 (m, 1H), 2.28-1.97 (m, 3H); 472.1 |
| 139 | 3 | 3-(tert-butyl)- | Column: Puningtech-Pntulips- | ¹H NMR (400 MHz, DMSO-d₆) δ 13.11 |
| | | 1-(2,2,2- | C18-10 μm-30*250 mm, Mobile | (bs, 1H), 8.72 (s, 1H), 8.41 (bs, 1H), |
| | | trifluoroethyl)- | phase: A: 0.1% FA/H2O B: ACN, | 8.12-8.02 (m, 3H), 7.90 (d, J = 8.4 Hz, |
| | | 1H-pyrazol-5- | Gradient (B %): 48% to 78% in 12 | 2H), 5.97 (s, 1H), 5.34 (bs, 1H), 4.84- |
| | | amine | min, Flow rate: 35 mL/min, UV | 4.68 (m, 2H), 3.78-3.69 (m, 1H), 3.63- |
| | | | wavelength: 220 nm | 3.55 (m, 1H), 2.46-2.31 (m, 1H), 2.12- |
| | | | | 1.84 (m, 3H), 1.19 (s, 9H); 534.2 |
| 140 | 3 | 1,3,5- | Column: “Puningtech-Pntulips- | ¹H NMR (400 MHz, DMSO-d₆) δ 13.06 |
| | | trimethyl-1H- | C18-10 μm-30*250 mm, Mobile | (bs, 1H), 8.73 (s, 1H), 8.10-7.98 (m, |
| | | pyrazol-4- | phase: A: 0.1% FA/H2O B: ACN, | 3H), 7.89 (d, J = 8.2 Hz, 2H), 7.28 (s, |
| | | amine | Gradient (B %): 20% to 60% in 15 | 1H), 5.27 (dd, J = 8.1, 3.5 Hz, 1H), 3.75- |
| | | | min, Flow rate: 35 mL/min, UV | 3.65 (m, 1H), 3.61-3.50 (m, 4H), |
| | | | wavelength: 220 nm | 2.42-2.30 (m, 1H), 2.08-1.97 (m, |
| | | | | 2H), 1.97-1.79 (m, 7H); 438.2 |
| 141 | 1 | 4- | Column: Agela-Durashell-C18-10 | ¹H NMR (400 MHz, DMSO-d₆) δ 13.12 |
| | | methoxycyclohexan- | μm-25*250 mm, Mobile phase: | (bs, 1H), 8.76-8.71 (m, 1H), 8.09- |
| | | 1-amine | A: 0.1% FA/H2O B: ACN, | 7.99 (m, 3H), 7.91 (d, J = 8.4 Hz, 2H), |
| | | | Gradient (B %): 34% to 64% in 12 | 5.78 (dd, J = 7.8, 13.6 Hz, 1H), 5.21 (dd, |
| | | | min, Flow rate: 30 mL/min, UV | J = 3.6, 7.9 Hz, 1H), 3.58-2.95 (m, |
| | | | wavelength: 220 nm | 6H), 2.35-2.20 (m, 1H), 2.17-1.22 (m, |
| | | | | 12H); 442.2 |
| 142 | 3 | 3- | Column: Welch-Xtimate-C18-7 | ¹H NMR (400 MHz, Methanol-d₄) δ |
| | | (difluoromethoxy)aniline | μm-30*250 mm, Mobile phase: | 8.64 (s, 1H), 8.13 (d, J = 8.4 Hz, 2H), |
| | | | A: 0.1% FA/H2O B: ACN, | 7.90 (dd, J = 11.2, 1.9 Hz, 1H), 7.78 (d, |
| | | | Gradient (B %): 35% to 75% in 13 | J = 8.5 Hz, 2H), 7.29 (s, 1H), 7.25-7.18 |
| | | | min, Flow rate: 35 mL/min, UV | (m, 2H), 6.78-6.61 (m, 1H), 6.73 (t, J = |
| | | | wavelength: 254 nm | 74.4 Hz, 1H), 5.52-5.38 (m, 1H), 3.93- |
| | | | | 3.80 (m, 1H), 3.77-3.60 (m, 1H), |
| | | | | 2.55-2.33 (m, 1H), 2.27-1.91 (m, |
| | | | | 3H); 472.1 |
| 143 | 1 | (4- | Column: Puningtech-Pntulips- | ¹H NMR (400 MHz, Methanol-d₄) δ |
| | | methylthiazol- | C18-10 μm-30*250 mm, Mobile | 8.63 (s, 1H), 8.13 (d, J = 8.2 Hz, 2H), |
| | | 2- | phase: A: 0.1% FA/H2O B: ACN, | 7.87 (dd, J = 11.0, 1.9 Hz, 1H), 7.78 (d, |
| | | yl)methanamine | Gradient (B %): 29% to 59% in 12 | J = 8.1 Hz, 2H), 6.96 (s, 1H), 5.37 (dd, |
| | | | min, Flow rate: 35 mL/min, UV | J = 8.2, 3.7 Hz, 1H), 4.59 (d, J = 16.5 Hz, |
| | | | wavelength: 220 nm | 1H), 4.47 (d, J = 16.6 Hz, 1H), 3.82- |
| | | | | 3.70 (m, 1H), 3.60 (dt, J = 9.3, 7.3 Hz, |
| | | | | 1H), 2.50-2.39 (m, 1H), 2.34 (s, 3H), |
| | | | | 2.22-1.96 (m, 3H); 441.2 |
| 144 | 1 | 1-(3- | Column: Phenomenex-Luna-C18- | ¹H NMR (400 MHz, Methanol-d₄) δ |
| | | (trifluoro- | 10 μm-25*250 mm, Mobile | 8.59 (d, J = 25.0 Hz, 1H), 8.13 (t, J = 8.9 |
| | | methyl)phenyl)ethan- | phase: A: 0.1% FA/H2O B: ACN, | Hz, 2H), 7.84 (ddd, J = 24.1, 11.6, 2.3 |
| | | 1-amine | Gradient (B %): 30% to 70% in 15 | Hz, 1H), 7.78 (d, J = 8.4 Hz, 1H), 7.73 |
| | | | min, Flow rate: 35 mL/min, UV | (d, J = 8.4 Hz, 1H), 7.67-7.40 (m, 4H), |
| | | | wavelength: 220 nm | 5.34 (ddd, J = 12.1, 7.8, 3.8 Hz, 1H), |
| | | | | 4.98-4.87 (m, 1H), 3.82-3.72 (m, |
| | | | | 1H), 3.64-3.53 (m, 1H), 2.48-2.33 |
| | | | | (m, 1H), 2.23-1.90 (m, 3H), 1.52- |
| | | | | 1.38 (m, 3H); 502.2 |
| 145 | 3 | 2- | Column: Puningtech-Pntulips- | ¹H NMR (400 MHz, Methanol-d₄) δ |
| | | cyclopro- | C18-10 μm-30*250 mm, Mobile | 8.68-8.62 (m, 3H), 8.12 (d, J = 8.4 Hz, |
| | | pylpyrimidin- | phase: A: 0.1% FA/H2O B: ACN, | 2H), 7.90 (d, J = 11.2 Hz, 1H), 7.81- |
| | | 5-amine | Gradient (B %): 30% to 60% in 12 | 7.74 (m, 2H), 5.46 (dd, J = 8.5, 4.0 Hz, |
| | | | min, Flow rate: 35 mL/min, UV | 1H), 3.94-3.81 (m, 1H), 3.78-3.63 |
| | | | wavelength: 220 nm | (m, 1H), 2.54-2.38 (m, 1H), 2.28- |
| | | | | 1.95 (m, 4H), 1.00 (d, J = 6.8 Hz, 4H); |
| | | | | 448.0 |
| 147 | 1 | 5,6,7,8- | Column: Phenomenex-Luna- | ¹H NMR (400 MHz, Methanol-d₄) δ |
| | | tetrahydroquinolin- | C18-10 μm-25*250 mm, Mobile | 8.66-8.57 (m, 1H), 8.36-8.22 (m, |
| | | 8-amine | phase: A: 0.1% FA/H2O B: ACN, | 1H), 8.18-8.11 (m, 2H), 7.95-7.82 |
| | | | Gradient (B %): 30% to 70% in 15 | (m, 1H), 7.82-7.74 (m, 2H), 7.55 (d, J = |
| | | | min, Flow rate: 35 mL/min, UV | 6.9 Hz, 1H), 7.26-7.15 (m, 1H), 5.48- |
| | | | wavelength: 220 nm | 5.29 (m, 1H), 4.77 (t, J = 5.9 Hz, 1H), |
| | | | | 3.77-3.53 (m, 2H), 2.98-2.70 (m, |
| | | | | 2H), 2.48-2.34 (m, 1H), 2.28-1.73 |
| | | | | (m, 7H); 461.2 |
| 149 | 2 | 6- | Column: Waters-SunFire-C18-10 | ¹H NMR (400 MHz, Methanol-d₄) δ |
| | | (trifluoro- | μm-19*250 mm, Mobile phase: | 8.74 (d, J = 2.5 Hz, 1H), 8.64 (s, 1H), |
| | | methyl)pyridin- | A: 0.1% FA/H2O B: ACN, | 8.17-8.08 (m, 3H), 7.91 (dd, J = 11.1, |
| | | 3-amine | Gradient (B %): 35% to 65% in 12 | 1.9 Hz, 1H), 7.78 (d, J = 8.5 Hz, 2H), |
| | | | min, Flow rate: 25 mL/min, UV | 7.66 (d, J = 8.7 Hz, 1H), 5.53-5.44 (m, |
| | | | wavelength: 220 nm | 1H), 3.99-3.82 (m, 1H), 3.82-3.67 |
| | | | | (m, 1H), 2.56-2.40 (m, 1H), 2.25- |
| | | | | 2.00 (m, 3H); 475.2 |
| 150 | 1 | (1,3-dimethyl- | Column: Phenomenex-Luna-C18- | ¹H NMR (400 MHz, Methanol-d₄) δ |
| | | 1H-pyrazol-5- | 10 μm-25*250 mm, Mobile | 8.61 (s, 1H), 8.14 (d, J = 8.4 Hz, 2H), |
| | | yl)methanamine | phase: A: 0.1% FA/H2O B: ACN, | 7.88 (dd, J = 11.2, 1.9 Hz, 1H), 7.78 (d, |
| | | | Gradient (B %): 30% to 70% in 15 | J = 8.5 Hz, 2H), 5.91 (s, 1H), 5.42-5.29 |
| | | | min, Flow rate: 35 mL/min, UV | (m, 1H), 4.37-4.14 (m, 2H), 3.75- |
| | | | wavelength: 220 nm | 3.65 (m, 4H), 3.53 (dt, J = 9.5, 7.3 Hz, |
| | | | | 1H), 2.48-2.36 (m, 1H), 2.16-1.93 |
| | | | | (m, 6H); 438.2 |

### Example 152: (S)-5-(4-(1-((3-fluoro-4-isopropylphenyl)carbamoyl)-3,3-dimethylazetidine-2-carboxamido)phenyl)-6-methylpicolinic acid & (R)-5-(4-(1-((3-fluoro-4-isopropylphenyl)carbamoyl)-3,3-dimethylazetidine-2-carboxamido)phenyl)-6-methylpicolinic acid

### Step 1: 3-fluoro-4-(prop-1-en-2-yl)aniline

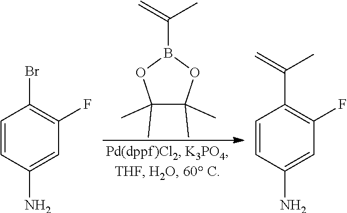

A solution of 4,4,5,5-tetramethyl-2-(prop-1-en-2-yl)-1,3,2-dioxaborolane (1.77 g, 10.5 mmol), Pd(dppf)Cl₂ (0.390 g, 0.526 mmol), K₃PO₄ (3.35 g, 15.8 mmol) and 4-bromo-3-fluoroaniline (1.00 g, 5.26 mmol) in THF (12 mL) and H₂O (3 mL) was stirred at 60° C. for 1 hour under nitrogen atmosphere. Upon completion, the resulting mixture was concentrated under reduced pressure. The crude residue was purified by silica gel column chromatography, eluted with PE/LA (1:1) to afford 3-fluoro-4-(prop-1-en-2-yl)aniline (600 mg, 75% yield) as a brown yellow oil. LCMS (ESI) [M+H]⁺: 152.1

### Step 2: 3-fluoro-4-isopropylaniline

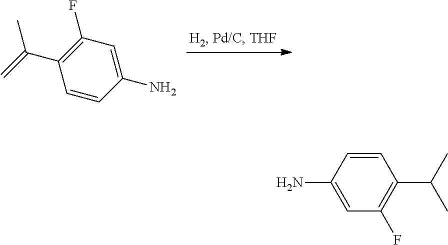

A solution of 3-fluoro-4-(prop-1-en-2-yl)aniline (600 mg, 3.97 mmol) and Pd/C (855 mg, 0.794 mmol, 10% on charcoal) in THF (50 mL) was stirred at room temperature for 1 hour under hydrogen atmosphere. Upon completion, the resulting mixture was filtered through celite. The filter cake was washed with ethyl acetate (6×10 mL). The filtrate was concentrated under reduced pressure. The crude residue was purified by silica gel column chromatography, eluted with PE/EA (1:1) to afford 3-fluoro-4-isopropylaniline (500 mg, 82% yield) as a yellow solid. LCMS (ESI) [M+H]⁺: 154.2

### Step 3: 1-((3-fluoro-4-isopropylphenyl)carbamoyl)-3,3-dimethylazetidine-2-carboxylic acid

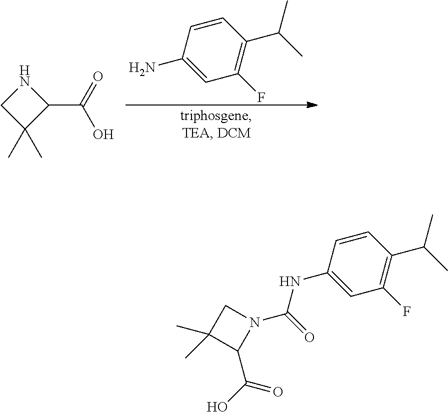

A solution of 3-fluoro-4-isopropylaniline (100 mg, 0.653 mmol) and 3,3-dimethylazetidine-2-carboxylic acid (126 mg, 0.980 mmol) and triethylamine (198 mg, 1.96 mmol) in DCM (5 mL) was treated with triphosgene (77.5 mg, 0.261 mmol) at room temperature under nitrogen atmosphere. After stirring for 1 hour, the mixture was concentrated under reduced pressure. The crude residue was purified by silica gel column chromatography, eluted with PE/EA (1:1) to afford 1-((3-fluoro-4-isopropylphenyl)carbamoyl)-3,3-dimethylazetidine-2-carboxylic acid (100 mg, 50% yield) as a white solid. LCMS (ESI) [M+H]⁺: 309.3

### Step 4: methyl 5-(4-(1-((3-fluoro-4-isopropylphenyl)carbamoyl)-3,3-dimethylazetidine-2-carboxamido)phenyl)-6-methylpicolinate

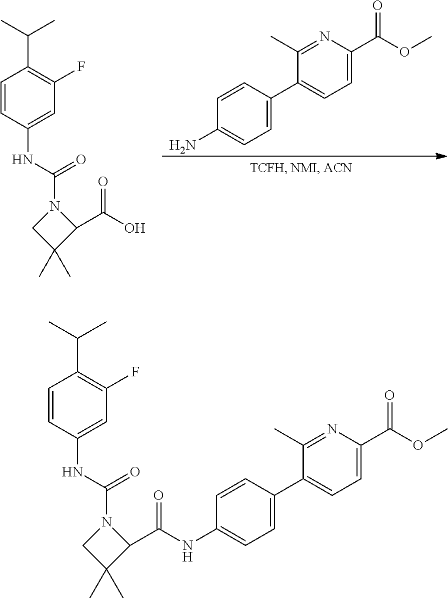 [SMILES: CC(C)c1ccc(NC(=O)N2CC(C)(C)C2C(=O)O)cc1F ; COC(=O)c1ccc(-c2ccc(N)cc2)c(C)n1 ; COC(=O)c1ccc(-c2ccc(NC(=O)C3N(C(=O)Nc4ccc(C(C)C)c(F)c4)CC3(C)C)cc2)c(C)n1]

A solution of methyl 5-(4-aminophenyl)-6-methylpyridine-2-carboxylate (78.6 mg, 0.324 mmol), 1-((3-fluoro-4-isopropylphenyl)carbamoyl)-3,3-dimethylazetidine-2-carboxylic acid (100 mg, 0.324 mmol), NMI (133 mg, 1.62 mmol) and TCFH (182 mg, 0.648 mmol) in ACN (5 mL) was stirred at room temperature for 1 hour under nitrogen atmosphere. Upon completion, the resulting mixture was concentrated under reduced pressure. The crude residue was purified by silica gel column chromatography, eluted with PE/EA (1:1) to afford methyl 5-(4-(1-((3-fluoro-4-isopropylphenyl)carbamoyl)-3,3-dimethylazetidine-2-carboxamido)phenyl)-6-methylpicolinate (80 mg, 46% yield) as a white solid. LCMS (ESI) [M+H]⁺: 533.6

### Step 5: 5-(4-(1-((3-fluoro-4-isopropylphenyl)carbamoyl)-3,3-dimethylazetidine-2-carboxamido)phenyl)-6-methylpicolinic acid

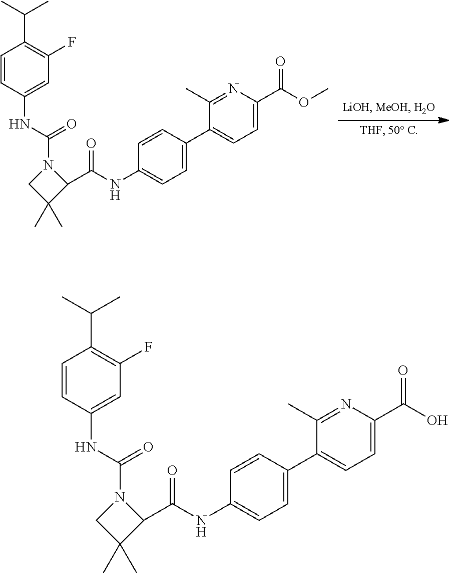

A solution of methyl 5-(4-(1-((3-fluoro-4-isopropylphenyl)carbamoyl)-3,3-dimethylazetidine-2-carboxamido)phenyl)-6-methylpicolinate (80 mg, 0.150 mmol) and LiOH monohydrate (19.7 mg, 0.470 mmol) in MeOH (1 mL), THF (2 mL) and H₂O (2 mL) was stirred at 50° C. for 1 hour under nitrogen atmosphere. Upon completion, the mixture was cooled to room temperature and acidified to pH=6 with HCl (1 M, aq.). The resulting mixture was concentrated under reduced pressure. The crude residue was purified by reversed-phase flash chromatography (conditions: column: C18 silica gel; mobile phase A: Water (0.1% FA), mobile phase B: MeCN; Gradient (B %): 10% to 80% B 15 min; detector, UV 254 nm) to afford 5-(4-(1-((3-fluoro-4-isopropylphenyl)carbamoyl)-3,3-dimethylazetidine-2-carboxamido)phenyl)-6-methylpicolinic acid (50.0 mg, 82% yield) as a white solid. LCMS (ESI) [M+H]⁺: 519.5

### Step 6: (S)-5-(4-(1-((3-fluoro-4-isopropylphenyl)carbamoyl)-3,3-dimethylazetidine-2-carboxamido)phenyl)-6-methylpicolinic acid & (R)-5-(4-(1-((3-fluoro-4-isopropylphenyl)carbamoyl)-3,3-dimethylazetidine-2-carboxamido)phenyl)-6-methylpicolinic acid

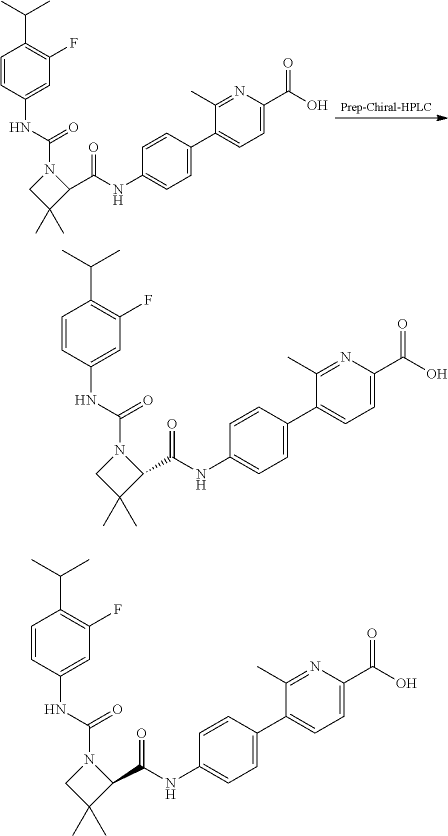

5-(4-(1-((3-fluoro-4-isopropylphenyl)carbamoyl)-3,3-dimethylazetidine-2-carboxamido)phenyl)-6-methylpicolinic acid (50 mg) was separated by prep Chiral-HPLC (conditions: Column: CHIRALPAK IG, 3*25 cm, 5 μm; Mobile Phase A: MTBE (0.1% FA), Mobile Phase B: MeOH; Flow rate: 40 mL/min; Gradient (B %): isocratic 25% B; Wave Length: 220/254 nm; RT1 (min): 5 min; RT2 (min): 14.3) to afford (Compound 152a; Method AP, Peak 1, 0.54 min) (S)-5-(4-(1-((3-fluoro-4-isopropylphenyl)carbamoyl)-3,3-dimethylazetidine-2-carboxamido)phenyl)-6-methylpicolinic acid (1^{st} peak, 15.1 mg, 30% yield) as a white solid and (Compound 152b; Method AP, Peak 2, 1.05 min) (R)-5-(4-(1-((3-fluoro-4-isopropylphenyl)carbamoyl)-3,3-dimethylazetidine-2-carboxamido)phenyl)-6-methylpicolinic acid (2^{nd} peak, 16.1 mg, 32% yield) as a white solid.

| Cmpd # | Characterization Data |
|---|---|
| 152a | LCMS (ESI) [M + H]+: 519.3 |
| | ¹H NMR (400 MHz, Methanol-d₄) δ 8.06 (d, J = 7.8 Hz, 1H), 7.85 (d, J = 7.4 Hz, 1H), 7.77 (d, J = |
| | 8.2 Hz, 2H), 7.38 (d, J = 7.5 Hz, 2H), 7.31 (d, J = 12.2 Hz, 1H), 7.20-7.10 (m, 2H), 4.51 (s, 1H), |
| | 3.86 (d, J = 7.3 Hz, 1H), 3.79 (d, J = 7.3 Hz, 1H), 3.21-3.08 (m, 1H), 2.57 (s, 3H), 1.47 (s, 3H), |
| | 1.26 (s, 3H), 1.25-1.16 (m, 6H). |
| 152b | LCMS (ESI) [M + H]⁺: 519.2 |
| | ¹H NMR (400 MHz, Methanol-d₄) δ 8.30-7.51 (m, 4H), 7.49-7.24 (m, 3H), 7.21-7.06 (m, 2H), |
| | 4.51 (s, 1H), 3.86 (s, 1H), 3.79 (d, J = 6.9 Hz, 1H), 3.13 (p, J = 6.9 Hz, 1H), 2.56 (bs, 3H), 1.47 (s, |
| | 3H), 1.26 (s, 3H), 1.24-1.12 (m, 6H). |

### Example 154: (S)-4-(5-fluoro-6-(1-((4-(1-(trifluoromethyl)cyclopropyl)phenyl)carbamoyl)pyrrolidin-2-yl)nicotinamido)benzoic acid & (R)-4-(5-fluoro-6-(1-((4-(1-(trifluoromethyl)cyclopropyl)phenyl)carbamoyl)pyrrolidin-2-yl)nicotinamido)benzoic acid

### Step 1: 6-(1-(tert-butoxycarbonyl)pyrrolidin-2-yl)-5-fluoronicotinic acid

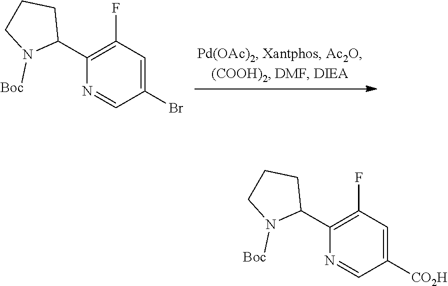 [SMILES: CC(C)(C)OC(=O)N1CCCC1c1ncc(Br)cc1F ; CC(C)(C)OC(=O)N1CCCC1c1ncc(C(=O)O)cc1F]

A mixture of tert-butyl 2-(5-bromo-3-fluoropyridin-2-yl)pyrrolidine-1-carboxylate (400 mg, 1.15 mmol), Pd(OAc)₂ (26.0 mg, 0.110 mmol), Xantphos (67.0 mg, 0.110 mmol), oxalic acid (1.00 g, 11.6 mmol), acetic anhydride (1.10 g, 11.6 mmol) and diisopropylethylamine (1.50 g, 11.6 mmol) in DMF (8.0 mL) was stirred at 110° C. for 2 hours under an atmosphere of carbon monoxide. Upon completion, the mixture was concentrated under reduced pressure. The crude residue was directly purified by reversed-phase flash chromatography (conditions: column: C18 silica gel; mobile phase A: water (0.1% formic acid), mobile phase B: acetonitrile; Gradient (B %): 0% to 100% over 25 min; detector, UV 254 nm) to afford 6-(1-(tert-butoxycarbonyl)pyrrolidin-2-yl)-5-fluoronicotinic acid (160 mg, 45% yield) as a white solid. LCMS (ESI) [M+H]⁺: 311.2

### Step 2: tert-butyl 2-(3-fluoro-5-((4-(methoxycarbonyl)phenyl)carbamoyl)pyridin-2-yl)pyrrolidine-1-carboxylate

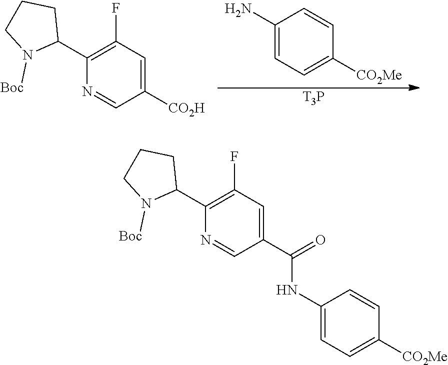

A solution of 6-(1-(tert-butoxycarbonyl)pyrrolidin-2-yl)-5-fluoronicotinic acid (150 mg, 0.480 mmol), methyl 4-aminobenzoate (90.0 mg, 0.590 mmol) and T₃P (307 mg, 0.960 mmol) in ethyl acetate (3.0 mL) was heated to 80° C. for 2 hours. Upon completion, the mixture was concentrated under reduced pressure. The crude residue was purified by reversed-phase flash chromatography (conditions: column: C18 silica gel; mobile phase A: water (0.1% formic acid), mobile phase B: acetonitrile; Gradient (B %): 0% to 100% over 25 min; detector, UV 254 nm) to afford tert-butyl 2-(3-fluoro-5-{[3-(methoxycarbonyl)phenyl]carbamoyl}pyridin-2-yl)pyrrolidine-1-carboxylate (130 mg, 61% yield) as a colorless oil. LCMS (ESI) [M+H]⁺: 444.1

### Step 3: methyl 4-(5-fluoro-6-(pyrrolidin-2-yl)nicotinamido)benzoate

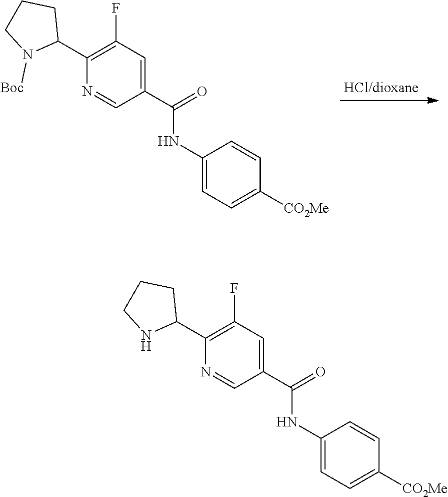 [SMILES: COC(=O)c1ccc(NC(=O)c2cnc(C3CCCN3C(=O)OC(C)(C)C)c(F)c2)cc1 ; COC(=O)c1ccc(NC(=O)c2cnc(C3CCCN3)c(F)c2)cc1]

To a solution of tert-butyl 2-(3-fluoro-5-((4-(methoxycarbonyl)phenyl)carbamoyl)pyridin-2-yl)pyrrolidine-1-carboxylate (130 mg, 0.290 mmol) in 1,4-dioxane (2.00 mL) at room temperature was added HCl (5.00 mL, 20 mmol, 4 M in 1,4-dioxane). The resultant mixture was stirred for 2 hours. Upon completion, the mixture was concentrated under reduced pressure to afford methyl 4-(5-fluoro-6-(pyrrolidin-2-yl)nicotinamido)benzoate (120 mg, crude) as a yellow solid, which was used directly in the next step without further purification. LCMS (ESI) [M+H]⁺: 301.0

### Step 4: methyl 4-(5-fluoro-6-(1-((4-(1-(trifluoromethyl)cyclopropyl)phenyl)carbamoyl)pyrrolidin-2-yl)nicotinamido)benzoate

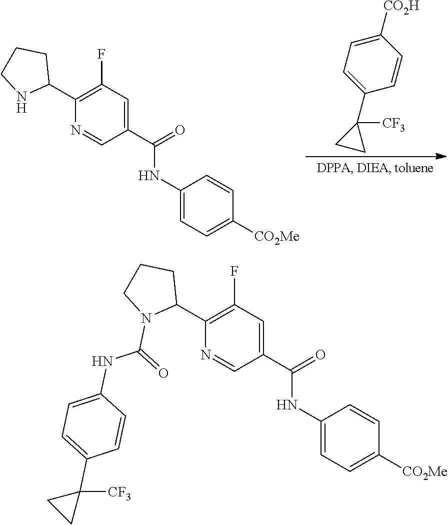

A solution of 4-(1-(trifluoromethyl)cyclopropyl)benzoic acid (100 mg, 0.430 mmol), DPPA (143 mg, 0.520 mmol) and diisopropylethylamine (224 mg, 1.73 mmol) in toluene (2.0 mL) at room temperature was stirred for 30 minutes. Then, methyl 4-(5-fluoro-6-(pyrrolidin-2-yl)nicotinamido)benzoate (149 mg, 0.430 mmol) was added. The reaction was heated to 80° C. for 2 hours. Upon completion, the mixture was concentrated under reduced pressure. The crude residue was purified by reversed-phase flash chromatography (conditions: column: C18 silica gel; mobile phase A: water (0.1% formic acid), mobile phase B: acetonitrile; Gradient (B %): 0% to 100% over 20 min; detector, UV 254 nm) to afford methyl 4-(5-fluoro-6-(1-((4-(1-(trifluoromethyl)cyclopropyl)phenyl)carbamoyl)pyrrolidin-2-yl)nicotinamido)benzoate (100 mg, 40%) as a yellow solid. LCMS (ESI) [M+H]⁺: 571.0

### Step 5: 4-(5-fluoro-6-(1-((4-(1-(trifluoromethyl)cyclopropyl)phenyl)carbamoyl)pyrrolidin-2-yl)nicotinamido)benzoic acid

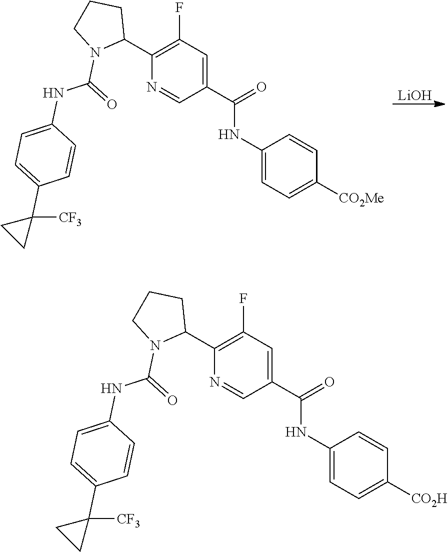 [SMILES: COC(=O)c1ccc(NC(=O)c2cnc(C3CCCN3C(=O)Nc3ccc(C4(C(F)(F)F)CC4)cc3)c(F)c2)cc1 ; "LiOH" ; O=C(O)c1ccc(NC(=O)c2cnc(C3CCCN3C(=O)Nc3ccc(C4(C(F)(F)F)CC4)cc3)c(F)c2)cc1]

A solution of methyl 4-(5-fluoro-6-(1-((4-(1-(trifluoromethyl)cyclopropyl)phenyl)carbamoyl)pyrrolidin-2-yl)nicotinamido)benzoate (201 mg, 0.350 mmol) and lithium hydroxide monohydrate (74.0 mg, 1.76 mmol) in THF (4.0 mL) and water (1.0 mL) was stirred at 40° C. for 2 hours. Upon completion, the mixture was concentrated under reduced pressure. The crude residue was purified by reversed-phase flash chromatography (conditions: column: C18 silica gel; mobile phase A: water (0.1% formic acid), mobile phase B: acetonitrile; Gradient (B %): 0% to 100% over 25 min; detector, UV 254 nm) to afford 4-(5-fluoro-6-(1-((4-(1-(trifluoromethyl)cyclopropyl)phenyl)carbamoyl)pyrrolidin-2-yl)nicotinamido)benzoic acid (50 mg, 26%) as an off-white solid. LCMS (ESI) [M+H]⁺: 557.2

### Step 6: (S)-4-(5-fluoro-6-(1-((4-(1-(trifluoromethyl)cyclopropyl)phenyl)carbamoyl)pyrrolidin-2-yl)nicotinamido)benzoic acid & (R)-4-(5-fluoro-6-(1-((4-(1-(trifluoromethyl)cyclopropyl)phenyl)carbamoyl)pyrrolidin-2-yl)nicotinamido)benzoic acid

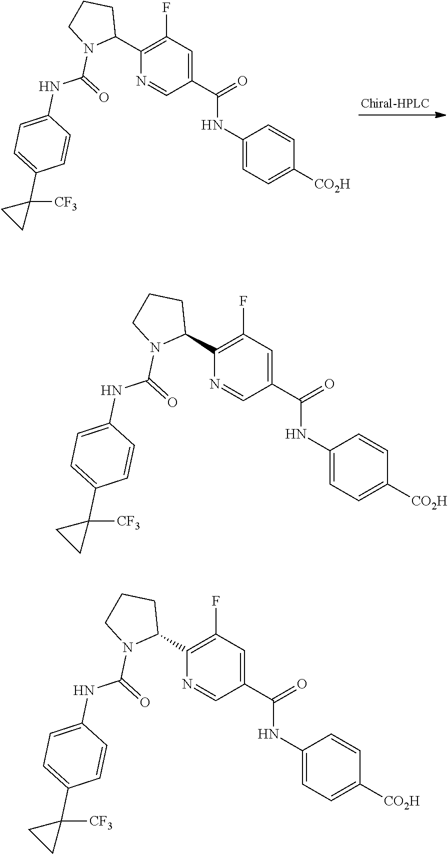 [SMILES: O=C(O)c1ccc(NC(=O)c2cnc(C3CCCN3C(=O)Nc3ccc(C4(C(F)(F)F)CC4)cc3)c(F)c2)cc1 ; O=C(O)c1ccc(NC(=O)c2cnc([C@@H]3CCCN3C(=O)Nc3ccc(C4(C(F)(F)F)CC4)cc3)c(F)c2)cc1 ; O=C(O)c1ccc(NC(=O)c2cnc([C@H]3CCCN3C(=O)Nc3ccc(C4(C(F)(F)F)CC4)cc3)c(F)c2)cc1]

4-(5-Fluoro-6-(1-((4-(1-(trifluoromethyl)cyclopropyl)phenyl)carbamoyl)pyrrolidin-2-yl)nicotinamido)benzoic acid (50 mg) was resolved by chiral HPLC (conditions: Column: CHIRALPAK IK 3*25 cm, 5 μm; Mobile Phase A: Hexanes (0.1% FA), Mobile Phase B: EtOH; Flow rate: 40 mL/min; Gradient (B %): isocratic 20% B; Wave Length: 220/254 nm; RT1 (min): 10.2; RT2 (min): 14.5) to afford (Compound 154a; Method P, Peak 1, 1.02 min) (S)-4-(5-fluoro-6-(1-((4-(1-(trifluoromethyl)cyclopropyl)phenyl)carbamoyl)pyrrolidin-2-yl)nicotinamido)benzoic acid (1^{st} peak, 15.2 mg, 35%) and (Compound 154b; Method P, Peak 2, 1.46 min) (R)-4-(5-fluoro-6-(1-((4-(1-(trifluoromethyl)cyclopropyl)phenyl)carbamoyl)pyrrolidin-2-yl)nicotinamido)benzoic acid (2^{nd} peak, 14.5 mg, 33%) as a white solid.

| Cmpd # | Characterization Data |
|---|---|
| 154a | LCMS (ESI) [M + H]⁺: 557.1 |
| | ¹H NMR (400 MHz, DMSO-d₆) δ 12.77 (s, 1H), 10.69 (s, 1H), 8.83 (s, 1H), 8.37 (s, 1H), 8.12 |
| | (dd, J = 10.6, 1.8 Hz, 1H), 7.94 (d, J = 8.8 Hz, 2H), 7.87 (d, J = 8.8 Hz, 2H), 7.44 (d, J = 8.7 Hz, |
| | 2H), 7.26 (d, J = 8.6 Hz, 2H), 5.35 (t, J = 6.4 Hz, 1H), 3.77 (q, J = 7.7 Hz, 1H), 3.64 (q, J = 7.7 |
| | Hz, 1H), 2.40-2.31 (m, 1H), 2.15-2.00 (m, 2H), 1.93-1.83 (m, 1H), 1.30-1.22 (m, 2H), |
| | 1.02 (s, 2H). |
| 154b | LCMS (ESI) [M + H]⁺: 557.1 |
| | ¹H NMR (400 MHz, DMSO-d₆) δ 12.75 (s, 1H), 10.69 (s, 1H), 8.83 (s, 1H), 8.36 (s, 1H), 8.12 |
| | (dd, J = 10.7, 1.8 Hz, 1H), 7.94 (d, J = 8.8 Hz, 2H), 7.87 (d, J = 8.8 Hz, 2H), 7.44 (d, J = 8.7 Hz, |
| | 2H), 7.26 (d, J = 8.6 Hz, 2H), 5.40-5.31 (m, 1H), 3.77 (q, J = 7.8 Hz, 1H), 3.64 (q, J = 8.2, 7.7 |
| | Hz, 1H), 2.40-2.31 (m, 1H), 2.15-1.98 (m, 2H), 1.94-1.82 (m, 1H), 1.30-1.21 (m, 2H), |
| | 1.02 (s, 2H). |

### Example 155: (S)-2-(5-(1H-benzo[d][1,2,3]triazol-5-yl)-3-fluoropyridin-2-yl)-N-(5-fluoro-6-(1-(trifluoromethyl)cyclopropyl)pyridin-3-yl)pyrrolidine-1-carboxamide & (R)-2-(5-(1H-benzo[d][1,2,3]triazol-5-yl)-3-fluoropyridin-2-yl)-N-(5-fluoro-6-(1-(trifluoromethyl)cyclopropyl)pyridin-3-yl)pyrrolidine-1-carboxamide

### Step 1: 5-chloro-3-fluoro-2-(pyrrolidin-2-yl)pyridine

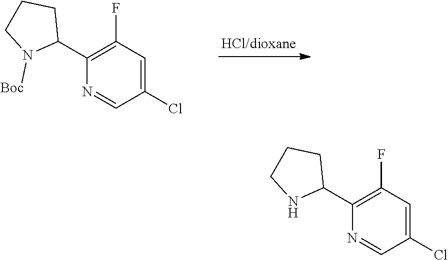 [SMILES: CC(C)(C)OC(=O)N1CCCC1c1ncc(Cl)cc1F ; Fc1cc(Cl)cnc1C1CCCN1]

To a solution of tert-butyl 2-(5-chloro-3-fluoropyridin-2-yl)pyrrolidine-1-carboxylate (500 mg, 1.66 mmol) in 1,4-dioxane (2.0 mL) at 0° C. was added HCl (8.0 mL, 32.0 mmol, 4 M in 1,4-dioxane). The resultant mixture was warmed to room temperature and stirred for 2 hours. Upon completion, the mixture was concentrated under reduced pressure to afford 5-chloro-3-fluoro-2-(pyrrolidin-2-yl)pyridine (300 mg, 90%) as a white solid, which was used directly in the next step without further purification. LCMS (ESI) [M+H]⁺: 201.1

### Step 2: 2-(5-chloro-3-fluoropyridin-2-yl)-N-(5-fluoro-6-(1-(trifluoromethyl)cyclopropyl)pyridin-3-yl)pyrrolidine-1-carboxamide

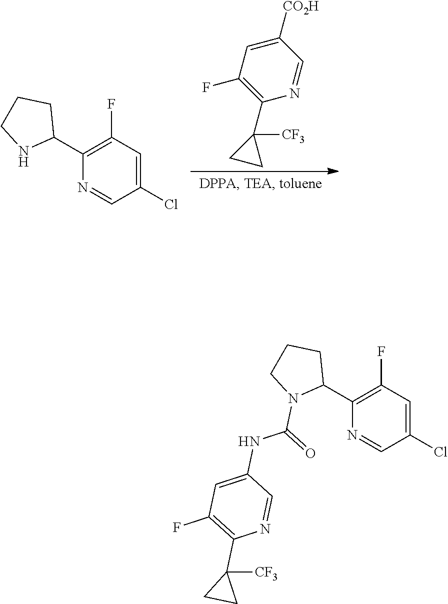 [SMILES: Fc1cc(Cl)cnc1C1CCCN1 ; O=C(O)c1cnc(C2(C(F)(F)F)CC2)c(F)c1 ; O=C(Nc1cnc(C2(C(F)(F)F)CC2)c(F)c1)N1CCCC1c1ncc(Cl)cc1F]

A solution of 5-fluoro-6-(1-(trifluoromethyl)cyclopropyl)nicotinic acid (223 mg, 0.898 mmol), DPPA (411 mg, 1.50 mmol) and triethylamine (227 mg, 2.24 mmol) in toluene (3.0 mL) was stirred at room temperature. After 30 minutes, 5-chloro-3-fluoro-2-(pyrrolidin-2-yl)pyridine (150 mg, 0.748 mmol) was added. The mixture was heated to 80° C. for 2 hours. Upon completion, the mixture was concentrated under reduced pressure. The crude residue was purified by silica gel column chromatography, eluted with petroleum ether/ethyl acetate (1:5), to afford 2-(5-chloro-3-fluoropyridin-2-yl)-N-(5-fluoro-6-(1-(trifluoromethyl)cyclopropyl)pyridin-3-yl)pyrrolidine-1-carboxamide (85 mg, 25%) as a brown oil. LCMS (ESI) [M+H]⁺: 428.2

### Step 3: 2-(5-(1H-benzo[d][1,2,3]triazol-5-yl)-3-fluoropyridin-2-yl)-N-(5-fluoro-6-(1-(trifluoromethyl)cyclopropyl)pyridin-3-yl)pyrrolidine-1-carboxamide

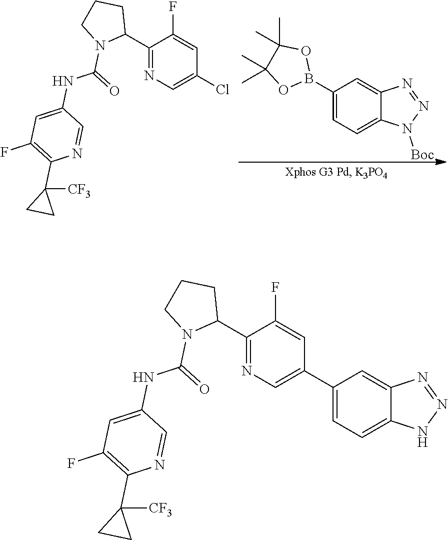

A solution of 2-(5-chloro-3-fluoropyridin-2-yl)-N-{5-fluoro-6-[1-(trifluoromethyl)cyclopropyl]pyridin-3-yl}pyrrolidine-1-carboxamide (80.0 mg, 0.179 mmol), tert-butyl 5-(4,4,5,5-tetramethyl-1,3,2-dioxaborolan-2-yl)-1,2,3-benzotriazole-1-carboxylate (92.7 mg, 0.268 mmol), tribasic potassium phosphate (114 mg, 0.537 mmol) and XPhos Pd G3 (30.3 mg, 0.036 mmol) in THF (2.0 mL) and water (0.20 mL) was stirred at 90° C. for 2 hours. Upon completion, the mixture was concentrated under reduced pressure. The crude residue was purified by reversed-phase flash chromatography (conditions: column: C18 silica gel; mobile phase A: water (0.1% formic acid), Mobile Phase B: acetonitrile; Gradient (B %): 20% to 90% over 25 min; detector: UV 254 nm) to afford 2-(5-(1H-benzo[d][1,2,3]triazol-5-yl)-3-fluoropyridin-2-yl)-N-(5-fluoro-6-(1-(trifluoromethyl)cyclopropyl)pyridin-3-yl)pyrrolidine-1-carboxamide (35 mg, 37% yield) as a white solid. LCMS (ESI) [M+H]⁺: 530.2

### Step 4: (S)-2-(5-(1H-benzo[d][1,2,3]triazol-5-yl)-3-fluoropyridin-2-yl)-N-(5-fluoro-6-(1-(trifluoromethyl)cyclopropyl)pyridin-3-yl)pyrrolidine-1-carboxamide & (R)-2-(5-(1H-benzo[d][1,2,3]triazol-5-yl)-3-fluoropyridin-2-yl)-N-(5-fluoro-6-(1-(trifluoromethyl)cyclopropyl)pyridin-3-yl)pyrrolidine-1-carboxamide

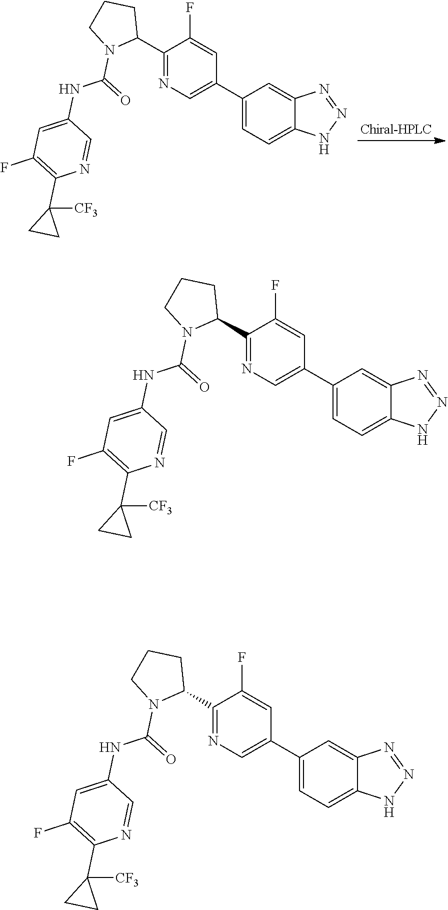 [SMILES: O=C(Nc1cnc(C2(C(F)(F)F)CC2)c(F)c1)N1CCCC1c1ncc(-c2ccc3[nH]nnc3c2)cc1F ; O=C(Nc1cnc(C2(C(F)(F)F)CC2)c(F)c1)N1CCC[C@H]1c1ncc(-c2ccc3[nH]nnc3c2)cc1F ; O=C(Nc1cnc(C2(C(F)(F)F)CC2)c(F)c1)N1CCC[C@@H]1c1ncc(-c2ccc3[nH]nnc3c2)cc1F]

2-(5-(1H-Benzo[d][1,2,3]triazol-5-yl)-3-fluoropyridin-2-yl)-N-(5-fluoro-6-(1-(trifluoromethyl)cyclopropyl)pyridin-3-yl)pyrrolidine-1-carboxamide (35 mg) was resolved by preparative chiral HPLC (conditions: Column: CHIRAL ART Cellulose-SC, 3*25 cm, 5 μm; Mobile Phase A: Hexanes (0.1% FA), Mobile Phase B: EtOH; Flow rate: 40 mL/min; Gradient (B %): isocratic 30% B; Wave Length: 220/254 nm; RT1 (min): 8.5; RT2 (min): 10.4) to afford (Compound 155a; Method AA, Peak 1, 1.09 min) (S)-2-(5-(1H-benzo[d][1,2,3]triazol-5-yl)-3-fluoropyridin-2-yl)-N-(5-fluoro-6-(1-(trifluoromethyl)cyclopropyl)pyridin-3-yl)pyrrolidine-1-carboxamide (1^{st} peak, 10.8 mg, 36%) as a white solid and (Compound 155b; Method AA, Peak 2, 1.54 min) (R)-2-(5-(1H-benzo[d][1,2,3]triazol-5-yl)-3-fluoropyridin-2-yl)-N-(5-fluoro-6-(1-(trifluoromethyl)cyclopropyl)pyridin-3-yl)pyrrolidine-1-carboxamide (2^{nd} peak 11.0 mg, 37%) as a white solid.

| Cmpd # | Characterization Data |
|---|---|
| 155a | LCMS (ESI) [M + H]⁺: 530.2 |
| | ¹H NMR (400 MHz, Methanol-d4) δ 8.67 (s, 1H), 8.43 (s, 1H), 8.16 (s, 1H), 8.02-7.92 (m, |
| | 2H), 7.89 (dd, J = 12.2, 2.2 Hz, 1H), 7.79 (d, J = 8.8 Hz, 1H), 5.52-5.45 (m, 1H), 3.95-3.85 |
| | (m, 1H), 3.79-3.69 (m, 1H), 2.47 (dt, J = 14.6, 7.5 Hz, 1H), 2.20 (s, 1H), 2.16-2.08 (m, 1H), |
| | 2.06 (s, 1H), 1.41 (q, J = 6.1, 5.4 Hz, 2H), 1.16 (s, 2H). |
| 155b | LCMS (ESI) [M + H]⁺: 530.2 |
| | ¹H NMR (400 MHz, Methanol-d4) δ 8.67 (s, 1H), 8.43 (s, 1H), 7.99-7.92 (m, 1H), 7.89 (dd, |
| | J = 12.2, 2.2 Hz, 4H), 5.48 (dd, J = 8.3, 4.1 Hz, 1H), 3.95-3.85 (m, 1H), 3.79-3.69 (m, 1H), |
| | 2.47 (dt, J = 15.1, 7.5 Hz, 1H), 2.20 (s, 3H), 1.44-1.31 (m, 2H), 1.16 (s, 2H). |

### Example 156: (R)-4-(5-fluoro-6-(1-((4-methyl-5-(1-(trifluoromethyl)cyclopropyl)pyridin-2-yl)carbamoyl)pyrrolidin-2-yl)pyridin-3-yl)benzoic acid

### Step 1: methyl 4-methyl-5-(3,3,3-trifluoroprop-1-en-2-yl)picolinate

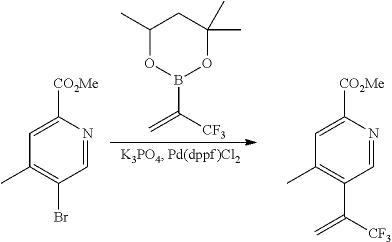

A mixture of methyl 5-bromo-4-methylpyridine-2-carboxylate (1.50 g, 6.52 mmol), 4,4,6-trimethyl-2-(3,3,3-trifluoroprop-1-en-2-yl)-1,3,2-dioxaborinane (1.45 g, 6.52 mmol), tribasic potassium phosphate (4.15 g, 19.6 mmol) and Pd(dppf)Cl₂ (0.480 g, 0.650 mmol) in THF (15.0 mL) and water (1.5 mL) was stirred at 80° C. for 3 hours under nitrogen atmosphere. Upon completion, the mixture was concentrated under reduced pressure. The crude residue was purified by silica gel column chromatography, eluted with petroleum ether/ethyl acetate (4:1), to afford methyl 4-methyl-5-(3,3,3-trifluoroprop-1-en-2-yl)picolinate (1.40 g, 88%) as a yellow solid. LCMS (ESI) [M+H]⁺: 246.0

### Step 2: methyl 4-methyl-5-(1-(trifluoromethyl)cyclopropyl)picolinate

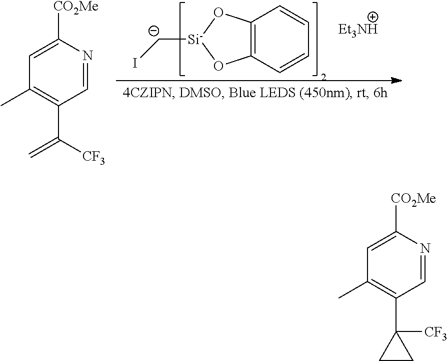

A mixture of methyl 4-methyl-5-(3,3,3-trifluoroprop-1-en-2-yl)picolinate (1.00 g, 4.07 mmol), 4CzIPN (0.030 g, 0.040 mmol) and triethylammonium bis(catecholato)iodomethylsilicate (2.36 g, 6.11 mmol) in DMSO (10.0 mL) at room temperature under nitrogen atmosphere was stirred under 450 nm blue light irradiation for 6 hours. Upon completion, the crude residue was purified by reversed-phase flash chromatography (conditions: column: C18 silica gel; mobile phase A: water (0.1% formic acid), mobile phase B: acetonitrile; Gradient (B %): 30% to 60% over 10 min; detector: UV 254 nm) to afford methyl 4-methyl-5-(1-(trifluoromethyl)cyclopropyl)picolinate (470 mg, 22%) as a yellow oil. LCMS (ESI) [M+H]⁺: 260.0

### Step 3: 4-methyl-5-(1-(trifluoromethyl)cyclopropyl)picolinic acid

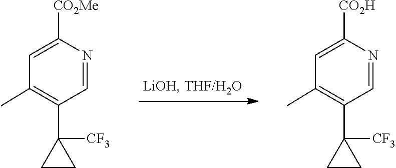 [SMILES: COC(=O)c1cc(C)c(C2(C(F)(F)F)CC2)cn1 ; Cc1cc(C(=O)O)ncc1C1(C(F)(F)F)CC1]

A mixture of methyl 4-methyl-5-(1-(trifluoromethyl)cyclopropyl)picolinate (460 mg, 1.77 mmol) and lithium hydroxide monohydrate (424 mg, 17.7 mmol) in THF (4.0 mL) and water (1.5 mL) was stirred overnight at room temperature. Upon completion, the mixture was concentrated under reduced pressure. The crude residue was purified by reversed-phase flash chromatography (conditions: column: C18 silica gel; mobile phase A: water (0.1% formic acid), mobile phase B: acetonitrile; Gradient B %: 10% to 95% over 18 min; detector, UV 254 nm) to afford 4-methyl-5-(1-(trifluoromethyl)cyclopropyl)picolinic acid (400 mg, 92%) as a white solid. LCMS (ESI) [M−H]⁻: 244.0

### Step 4: methyl (R)-4-(5-fluoro-6-(1-((4-methyl-5-(1-(trifluoromethyl)cyclopropyl)pyridin-2-yl)carbamoyl)pyrrolidin-2-yl)pyridin-3-yl)benzoate

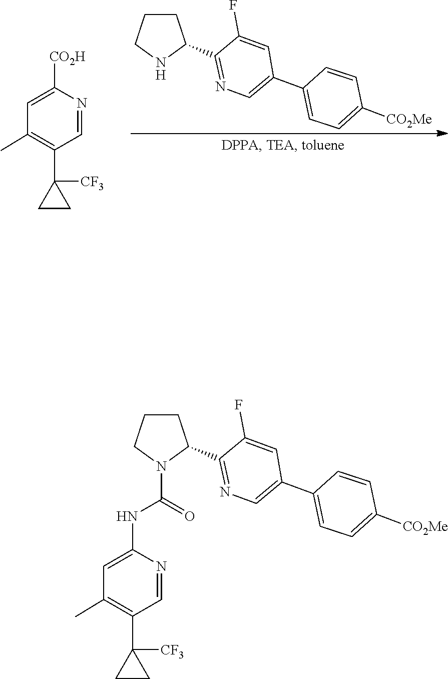 [SMILES: Cc1cc(C(=O)O)ncc1C1(C(F)(F)F)CC1 ; COC(=O)c1ccc(-c2cnc([C@H]3CCCN3)c(F)c2)cc1 ; COC(=O)c1ccc(-c2cnc([C@H]3CCCN3C(=O)Nc3cc(C)c(C4(C(F)(F)F)CC4)cn3)c(F)c2)cc1]

A mixture of methyl (R)-4-(5-fluoro-6-(pyrrolidin-2-yl)pyridin-3-yl)benzoate (100 mg, 0.330 mmol), 4-methyl-5-(1-(trifluoromethyl)cyclopropyl)picolinic acid (81.0 mg, 0.330 mmol), triethylamine (101 mg, 0.99 mmol), and DPPA (274 mg, 0.990 mmol) in toluene (3.0 mL) was stirred at 80° C. for 3 hours under nitrogen atmosphere. Upon completion, the mixture was concentrated under reduced pressure. The crude residue was purified by silica gel column chromatography, eluted with petroleum ether/ethyl acetate (6:1), to afford methyl (R)-4-(5-fluoro-6-(1-((4-methyl-5-(1-(trifluoromethyl)cyclopropyl)pyridin-2-yl)carbamoyl)pyrrolidin-2-yl)pyridin-3-yl)benzoate (40 mg, 22%) as a white solid. LCMS (ESI) [M+H]⁺: 543.1

### Step 5: (R)-4-(5-fluoro-6-(1-((4-methyl-5-(1-(trifluoromethyl)cyclopropyl)pyridin-2-yl)carbamoyl)pyrrolidin-2-yl)pyridin-3-yl)benzoic acid

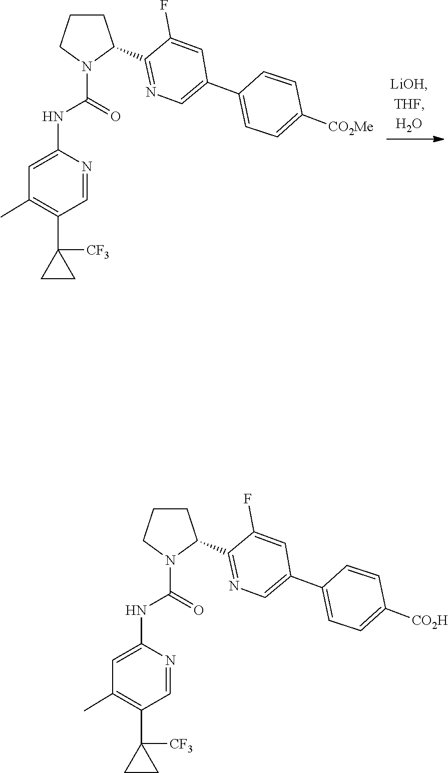 [SMILES: COC(=O)c1ccc(-c2cnc([C@H]3CCCN3C(=O)Nc3cc(C)c(C4(C(F)(F)F)CC4)cn3)c(F)c2)cc1 ; Cc1cc(NC(=O)N2CCC[C@@H]2c2ncc(-c3ccc(C(=O)O)cc3)cc2F)ncc1C1(C(F)(F)F)CC1]

A mixture of methyl (R)-4-(5-fluoro-6-(1-((4-methyl-5-(1-(trifluoromethyl)cyclopropyl)pyridin-2-yl)carbamoyl)pyrrolidin-2-yl)pyridin-3-yl)benzoate (40.0 mg, 0.090 mmol) and lithium hydroxide monohydrate (38.6 mg, 0.92 mmol) in THF (2.0 mL) and water (0.40 mL) was stirred at room temperature overnight. Upon completion, the mixture was concentrated under reduced pressure. The crude residue was purified by reversed-phase flash chromatography (conditions: column: C18 silica gel; mobile phase A: water (0.1% formic acid), mobile phase B: acetonitrile Gradient (B %): 0% to 100% over 30 min; detector: UV 254 nm) to afford (Compound 156) (R)-4-(5-fluoro-6-(1-((4-methyl-5-(1-(trifluoromethyl)cyclopropyl)pyridin-2-yl)carbamoyl)pyrrolidin-2-yl)pyridin-3-yl)benzoic acid (18.9 mg, 39%) as a white solid.

| Cmpd | |
|---|---|
| # | Characterization Data |
| 156 | LCMS (ESI) [M + H]⁺: 529.2 |
| | ¹H NMR (400 MHz, Methanol-d4) δ 8.64 (s, 1H), |
| | 8.16 (s, 1H), 8.13 (d, J = 8.4 Hz, 2H), 7.92 (dd, J = |
| | 11.2, 1.9 Hz, 1H), 7.78 (d, J = 8.4 Hz, 2H), 7.67 (s, |
| | 1H), 5.53-5.40 (m, 1H), 3.96-3.82 (m, 1H), 3.80-3.70 (m, 1H), |
| | 2.58-2.44 (m, 1H), 2.39 (s, 3H), 2.23-2.02 (m, 3H), 1.48-1.42 |
| | (m, 2H), 1.10-1.01 (m, 2H). |

### Example 157: (R)-4-(6-(1-((2,2-difluoro-[1,3]dioxolo[4,5-b]pyridin-5-yl)carbamoyl)pyrrolidin-2-yl)-5-fluoropyridin-3-yl)benzoic acid

### Step 1: 6-bromopyridine-2,3-diol

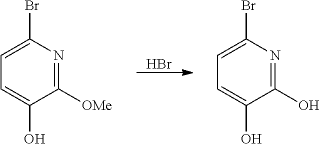 [SMILES: COc1nc(Br)ccc1O ; Br ; Oc1ccc(Br)nc1O]

A mixture of 6-bromo-2-methoxypyridin-3-ol (10.0 g, 49.0 mmol) in hydrobromic acid (20.0 mL, 40% aqueous) was stirred at 100° C. overnight. Upon completion, the mixture was cooled to room temperature and the precipitated solids were collected by filtration, washed with water (2×50 mL) and dried to afford 6-bromopyridine-2,3-diol (8.0 g, 86%) as a yellow solid. LCMS (ESI) [M+H]⁺: 190.0

### Step 2: 5-bromo-[1,3]dioxolo[4,5-b]pyridine-2-thione

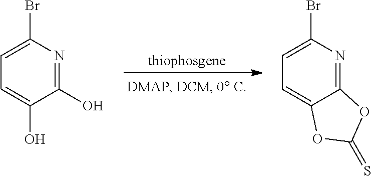 [SMILES: Oc1ccc(Br)nc1O ; "thiophosgene" ; S=c1oc2ccc(Br)nc2o1]

A mixture of 6-bromopyridine-2,3-diol (8.00 g, 42.1 mmol), thiophosgene (5.33 g, 46.3 mmol) and DMAP (10.3 g, 84.2 mmol) in dichloromethane (150 mL) was stirred at 0° C. for 3 hours under nitrogen atmosphere. Upon completion, the mixture was poured into water (100 mL) and extracted with DCM (2×40 mL). The combined organic layers were concentrated under reduced pressure in an ice bath temperature. The crude residue was purified by silica gel column chromatography, eluted with petroleum ether/ethyl acetate (8:1) to afford 5-bromo-[1,3]dioxolo[4,5-b]pyridine-2-thione (5.00 g, 51%) as a white solid.

### Step 3: 5-bromo-2,2-difluoro-[1,3]dioxolo[4,5-b]pyridine

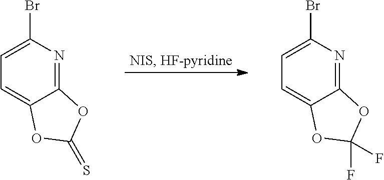 [SMILES: S=c1oc2ccc(Br)nc2o1 ; "NIS, HF-pyridine" ; FC1(F)Oc2ccc(Br)nc2O1]

A mixture of 5-bromo-[1,3]dioxolo[4,5-b]pyridine-2-thione (5.00 g, 21.5 mmol), N-iodosuccinimide (24.2 g, 108 mmol) and pyridine hydrofluoride (42.7 g, 431 mmol) in dichloromethane (300 mL) was stirred at −78° C. for 4 hours. Upon completion, the mixture was concentrated under reduced pressure in an ice bath temperature. The crude residue was purified by silica gel column chromatography, eluted with petroleum ether/ethyl acetate (8:1), to afford 5-bromo-2,2-difluoro-[1,3]dioxolo[4,5-b]pyridine (5.00 g, 97%) as a yellow oil.

### Step 4: tert-butyl (2,2-difluoro-[1,3]dioxolo[4,5-b]pyridin-5-yl)carbamate

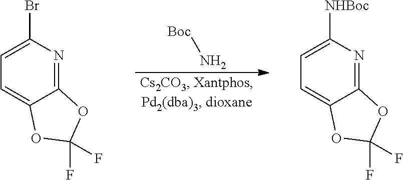

A solution of 5-bromo-2,2-difluoro-[1,3]dioxolo[4,5-b]pyridine (5.00 g, 21.0 mmol), Pd₂(dba)₃ (1.92 g, 2.10 mmol), XantPhos (1.22 g, 2.10 mmol), tert-butyl carbamate (4.92 g, 4.20 mmol) and Cs₂CO₃ (20.5 g, 63.0 mmol) in 1,4-dioxane (50.0 mL) was stirred at 90° C. for 3 hours under nitrogen atmosphere. Upon completion, the reaction mixture was concentrated under reduced pressure. The crude residue was purified by silica gel column chromatography, eluted with petroleum ether/ethyl acetate (8:1), to afford tert-butyl (2,2-difluoro-[1,3]dioxolo[4,5-b]pyridin-5-yl)carbamate (1.2 g, 21%) as a white solid. LCMS (ESI) [M+H]⁺: 275.0

### Step 5: 2,2-difluoro-[1,3]dioxolo[4,5-b]pyridin-5-amine

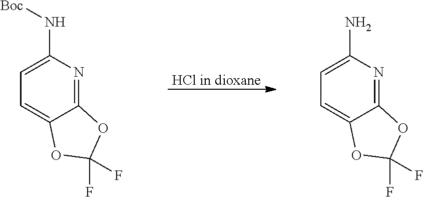 [SMILES: CC(C)(C)OC(=O)Nc1ccc2c(n1)OC(F)(F)O2 ; "HCl in dioxane" ; Nc1ccc2c(n1)OC(F)(F)O2]

To a solution of tert-butyl N-{2,2-difluoro-[1,3]dioxolo[4,5-b]pyridin-5-yl}carbamate (1.2 g, 4.37 mmol) in dioxane (10.0 mL) was added HCl (30 mL, 120 mmol, 4 M in 1,4-dioxane), and the resultant mixture was stirred at room temperature for 2 hours. Upon completion, the mixture was concentrated under reduced pressure to afford crude 2,2-difluoro-[1,3]dioxolo[4,5-b]pyridin-5-amine (550 mg, 72%, crude) as a white solid, which was used directly in the next step without further purification. LCMS (ESI) [M+H]⁺:175.0

### Step 6: (R)-4-(6-(1-((2,2-difluoro-[1,3]dioxolo[4,5-b]pyridin-5-yl)carbamoyl)pyrrolidin-2-yl)-5-fluoropyridin-3-yl)benzoic acid

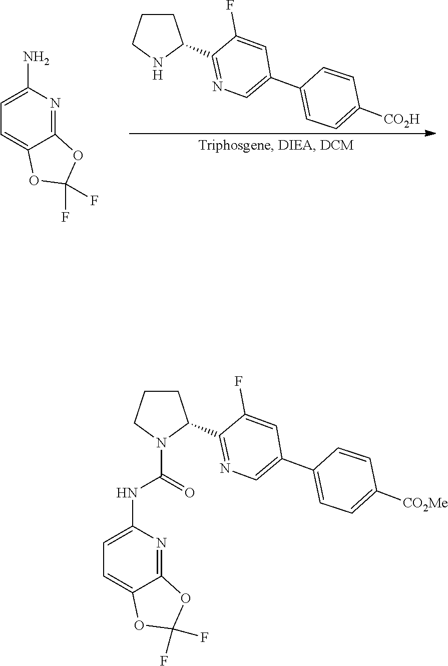 [SMILES: Nc1ccc2c(n1)OC(F)(F)O2 ; O=C(O)c1ccc(-c2cnc([C@H]3CCCN3)c(F)c2)cc1 ; COC(=O)c1ccc(-c2cnc([C@H]3CCCN3C(=O)Nc3ccc4c(n3)OC(F)(F)O4)c(F)c2)cc1]

A mixture of 2,2-difluoro-[1,3]dioxolo[4,5-b]pyridin-5-amine (50.0 mg, 0.280 mmol), (R)-4-(5-fluoro-6-(pyrrolidin-2-yl)pyridin-3-yl)benzoic acid (82.0 mg, 0.280 mmol), triphosgene (11.0 mg, 0.110 mmol) and diisopropylethylamine (185 mg, 1.43 mmol) in dichloromethane (3.0 mL) was stirred at room temperature for 2 hours under nitrogen atmosphere. Upon completion, the mixture was concentrated under reduced pressure. The crude residue was purified by reversed-phase flash chromatography (conditions: column: C18 silica gel; mobile phase A: water (0.1% formic acid), mobile phase B: acetonitrile; Gradient (B %): 10% to 60% over 19 min; detector, UV 254 nm) to afford (R)-4-(6-(1-((2,2-difluoro-[1,3]dioxolo[4,5-b]pyridin-5-yl)carbamoyl)pyrrolidin-2-yl)-5-fluoropyridin-3-yl)benzoic acid (48.9 mg, 35%) as a white solid.

| Cmpd # | Characterization Data |
|---|---|
| 157 | LCMS (ESI) [M + H]⁺: 487.1 |
| | ¹H NMR (400 MHz, Methanol-d4) δ 8.64 (s, 1H), 8.13 (d, J = 8.4 Hz, 2H), 7.91 (dd, J = 11.2, 1.9 |
| | Hz, 1H), 7.78 (d, J = 8.4 Hz, 2H), 7.56 (d, J = 8.7 Hz, 1H), 7.48 (d, J = 8.7 Hz, 1H), 5.52-5.38 (m, |
| | 1H), 3.93-3.83 (m, 1H), 3.77-3.67 (m, 1H), 2.54-2.38 (m, 1H), 2.26-2.01 (m, 3H). |

### Example 158: (R)-4-(6-(1-((2,2-difluoro-[1,3]dioxolo[4,5-b]pyridin-6-yl)carbamoyl)pyrrolidin-2-yl)-5-fluoropyridin-3-yl)benzoic acid

### Step 1: 6-bromo-[1,3]dioxolo[4,5-b]pyridine-2-thione

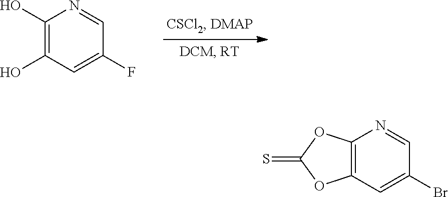 [SMILES: Oc1cc(F)cnc1O ; S=c1oc2cc(Br)cnc2o1]

Thiophosgene (9.07 g, 78.9 mmol) was added dropwise to a mixture of 5-bromopyridine-2,3-diol (10.0 g, 52.9 mmol) and DMAP (16.1 g, 132 mmol) in dichloromethane (120 mL) at 0° C. The resultant mixture was warmed to room temperature and stirred for 4 hours. Upon completion, the mixture was concentrated under reduced pressure. The crude residue was purified by silica gel column chromatography, eluted with petroleum ether/ethyl acetate (5:1), to afford 6-bromo-[1,3]dioxolo[4,5-b]pyridine-2-thione (10.0 g, 82%) as a white solid.

### Step 2: 6-bromo-2,2-difluoro-[1,3]dioxolo[4,5-b]pyridine

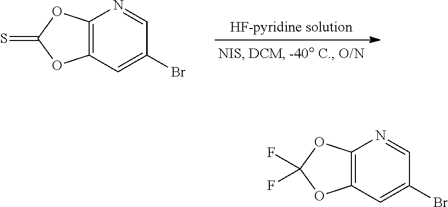 [SMILES: S=c1oc2cc(Br)cnc2o1 ; FC1(F)Oc2cc(Br)cnc2O1]

To a mixture of 6-bromo-[1,3]dioxolo[4,5-b]pyridine-2-thione (1.70 g, 7.32 mmol) in dichloromethane (20.0 mL) at −40° C. was added HF·Pyridine (7.26 g, 73.3 mmol) followed by the portion-wise addition of N-iodosuccinimide (8.24 g, 36.6 mmol). The resultant mixture was stirred at −40° C. for 1 hour and 30 minutes, was warmed to room temperature and stirred for an additional 1 hour and 30 minutes. Upon completion, the mixture was concentrated under reduced pressure. The crude residue was purified by silica gel column chromatography, eluted with petroleum ether/ethyl acetate (10:1), to afford 6-bromo-2,2-difluoro-[1,3]dioxolo[4,5-b]pyridine (600 mg, 34%) as a yellow solid.

### Step 3: tert-butyl (2,2-difluoro-[1,3]dioxolo[4,5-b]pyridin-6-yl)carbamate

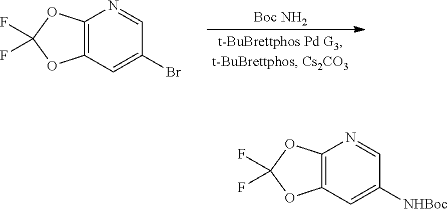 [SMILES: FC1(F)Oc2cc(Br)cnc2O1 ; "Boc NH2" ; CC(C)(C)OC(=O)Nc1cnc2c(c1)OC(F)(F)O2]

A solution of 6-bromo-2,2-difluoro-[1,3]dioxolo[4,5-b]pyridine (590 mg, 2.47 mmol), tert-butyl carbamate (436 mg, 3.71 mmol), t-BuBrettphos Pd G3 (212 mg, 0.240 mmol), t-BuBrettphos (240 mg, 0.490 mmol), and cesium carbonate (1.61 g, 4.95 mmol) in 1,4-dioxane (10.0 mL) was stirred at 60° C. for 12 hours under nitrogen atmosphere. Upon completion, the mixture was concentrated under reduced pressure. The crude residue was directly purified by silica gel column chromatography, eluted with petroleum ether/ethyl acetate (5:1), to afford tert-butyl (2,2-difluoro-[1,3]dioxolo[4,5-b]pyridin-6-yl)carbamate (210 mg, 31%) as a pale-yellow solid. LCMS (ESI) [M−H]⁻: 273.1

### Step 4: 2,2-difluoro-[1,3]dioxolo[4,5-b]pyridin-6-amine

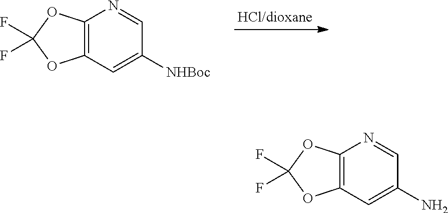 [SMILES: CC(C)(C)OC(=O)Nc1cnc2c(c1)OC(F)(F)O2 ; "HCl/dioxane" ; Nc1cnc2c(c1)OC(F)(F)O2]

A solution of tert-butyl (2,2-difluoro-[1,3]dioxolo[4,5-b]pyridin-6-yl)carbamate (200 mg, 0.720 mmol) in HCl/1,4-dioxane (6 mL, 1.0 M) was stirred at room temperature for 4 hours. Upon completion, the mixture was concentrated under reduced pressure to afford 2,2-difluoro-[1,3]dioxolo[4,5-b]pyridin-6-amine (170 mg, crude) as a white solid, which was used directly in the next step without further purification. LCMS (ESI) [M+H]⁺: 175.0

### Step 5: (R)-4-(6-(1-((2,2-difluoro-[1,3]dioxolo[4,5-b]pyridin-6-yl)carbamoyl)pyrrolidin-2-yl)-5-fluoropyridin-3-yl)benzoic acid

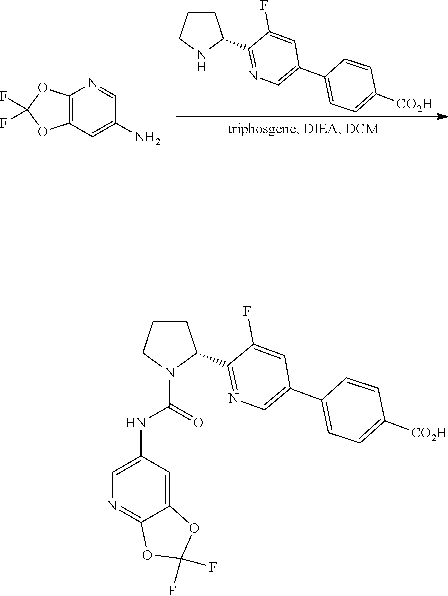 [SMILES: Nc1cnc2c(c1)OC(F)(F)O2 ; O=C(O)c1ccc(-c2cnc([C@H]3CCCN3)c(F)c2)cc1 ; O=C(O)c1ccc(-c2cnc([C@H]3CCCN3C(=O)Nc3cnc4c(c3)OC(F)(F)O4)c(F)c2)cc1]

To a mixture of 2,2-difluoro-[1,3]dioxolo[4,5-b]pyridin-6-amine (50 mg, 0.280 mmol) in dichloromethane (3.0 mL) at 0° C. was added diisopropylethylamine (186 mg, 1.43 mmol) followed by the dropwise addition of triphosgene (34 mg, 0.110 mmol) at 0° C. After 30 minutes, (R)-4-(5-fluoro-6-(pyrrolidin-2-yl)pyridin-3-yl)benzoic acid (82.0 mg, 0.280 mmol) was added dropwise over 3 minutes. The resultant mixture was warmed to room temperature and stirred for 2 hours. Upon completion, the reaction was cooled in an ice bath and quenched with ice water. The mixture was extracted with ethyl acetate (3×10 mL). The combined organic layers were dried over anhydrous sodium sulfate, filtered, and concentrated under reduced pressure. The crude residue was purified by preparative HPLC (conditions: column: XSelect CSH Fluoro Phenyl 30*150 mm, 5 μm; mobile phase A: water (0.1% formic acid); mobile phase B: acetonitrile; gradient (B %): 34% to 54% over 15 min; UV wavelength: 254 nm/220 nm; RT1 (min): 9.6) to afford (R)-4-(6-(1-((2,2-difluoro-[1,3]dioxolo[4,5-b]pyridin-6-yl)carbamoyl)pyrrolidin-2-yl)-5-fluoropyridin-3-yl)benzoic acid (25.5 mg, 18%) as a white solid.

| Cmpd # | Characterization Data |
|---|---|
| 158 | LCMS (ESI) [M + H]⁺: 487.1 |
| | ¹H NMR (400 MHz, DMSO-d₆) δ 8.74 (s, 1H), 8.15 (s, 1H), 8.09-7.99 (m, 3H), 7.89 (d, J = 8.0 |
| | Hz, 2H), 7.31 (d, J = 8.1 Hz, 2H), 7.03 (d, J = 8.1 Hz, 2H), 4.84 (d, J = 6.1 Hz, 1H), 3.73 (t, J = |
| | 6.8 Hz, 2H), 2.83-2.71 (m, 1H), 2.38-2.26 (m, 1H), 2.26-2.14 (m, 1H), 1.78-1.67 (m, 1H), |
| | 1.13 (t, J = 6.6 Hz, 9H). |

### Example 159: (R)-4-(5-fluoro-6-(1-((5-fluoro-6-(1-(trifluoromethyl)cyclopropyl)pyridin-3-yl)carbamoyl)piperidin-2-yl)pyridin-3-yl)benzoic acid & (S)-4-(5-fluoro-6-(1-((5-fluoro-6-(1-(trifluoromethyl)cyclopropyl)pyridin-3-yl)carbamoyl)piperidin-2-yl)pyridin-3-yl)benzoic acid

### Step 1: tert-butyl 2-(5-chloro-3-fluoropyridin-2-yl)piperidine-1-carboxylate

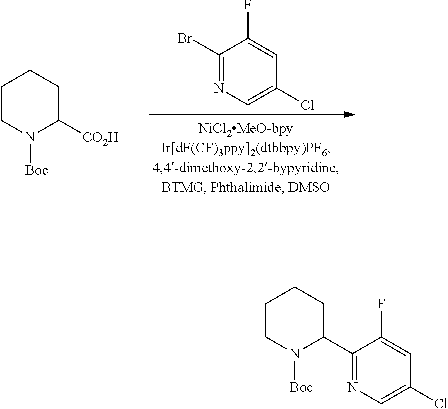 [SMILES: CC(C)(C)OC(=O)N1CCCCC1C(=O)O ; Fc1cc(Cl)cnc1Br ; CC(C)(C)OC(=O)N1CCCCC1c1ncc(Cl)cc1F]

A solution of 1-(tert-butoxycarbonyl)piperidine-2-carboxylic acid (1.00 g, 4.36 mmol), 2-bromo-5-chloro-3-fluoropyridine (1.10 g, 5.23 mmol), dichloronickel;1,2-dimethoxyethane (96 mg, 0.430 mmol), 4,4′-dimethoxy-2,2′-bipyridine (75 mg, 0.349 mmol), N″-tert-butyl-N,N,N′,N′-tetramethylguanidine (1.12 g, 6.54 mmol), Ir[dF(CF₃)ppy]₂(dtbpy))PF₆ (98 mg, 87.0 μmol) and 2,3-dihydro-1H-isoindole-1,3-dione (642 mg, 4.36 mmol) in DMSO (15.0 mL) at room temperature was stirred under 450 nm blue LED irradiation for 24 hours. Upon completion, the mixture was diluted with water and extracted with ethyl acetate (3×60 mL). The combined extracts were washed with brine, dried over anhydrous sodium sulfate, filtered and concentrated under reduced pressure. The crude residue was purified by reversed-phase flash chromatography (conditions: column: C18 silica gel; mobile phase A: water (0.1% formic acid), mobile phase B: acetonitrile; Gradient (B %): 0% to 100% over 25 min; detector: UV 254 nm) to afford tert-butyl 2-(5-chloro-3-fluoropyridin-2-yl)piperidine-1-carboxylate (361 mg, 26% yield) as a white solid. LCMS (ESI) [M+H]⁺: 315.1

### Step 2: tert-butyl 2-(5-(4-(tert-butoxycarbonyl)phenyl)-3-fluoropyridin-2-yl)piperidine-1-carboxylate

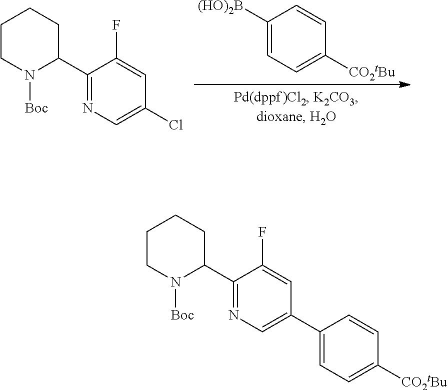 [SMILES: CC(C)(C)OC(=O)N1CCCCC1c1ncc(Cl)cc1F ; CC(C)(C)OC(=O)c1ccc(B(O)O)cc1 ; CC(C)(C)OC(=O)c1ccc(-c2cnc(C3CCCCN3C(=O)OC(C)(C)C)c(F)c2)cc1]

A solution of tert-butyl 2-(5-chloro-3-fluoropyridin-2-yl)piperidine-1-carboxylate (340 mg, 1.08 mmol), (4-(tert-butoxycarbonyl)phenyl)boronic acid (360 mg, 1.62 mmol), Pd(dppf)Cl₂ (79.0 mg, 0.100 mmol) and potassium carbonate (448 mg, 3.24 mmol) in dioxane (5.0 mL) and water (1.0 mL) was stirred at 80° C. for 2 hours under nitrogen atmosphere. Upon completion, the mixture was cooled to room temperature and concentrated under reduced pressure. The crude residue was purified by silica gel column chromatography, eluted with petroleum ether/ethyl acetate (1:1), to afford tert-butyl 2-(5-(4-(tert-butoxycarbonyl)phenyl)-3-fluoropyridin-2-yl)piperidine-1-carboxylate (320 mg, 65% yield) as a yellow solid. LCMS (ESI) [M+H]⁺: 457.2

### Step 3: 4-(5-fluoro-6-(piperidin-2-yl)pyridin-3-yl)benzoic acid

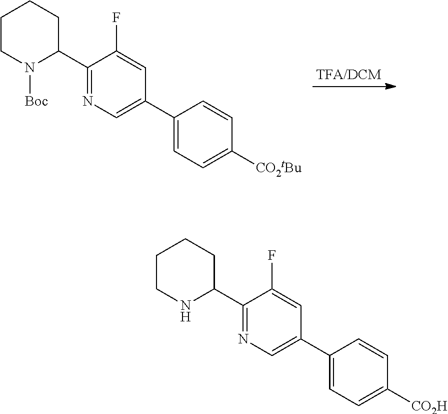 [SMILES: CC(C)(C)OC(=O)c1ccc(-c2cnc(C3CCCCN3C(=O)OC(C)(C)C)c(F)c2)cc1 ; O=C(O)c1ccc(-c2cnc(C3CCCCN3)c(F)c2)cc1]

To a mixture of tert-butyl 2-(5-(4-(tert-butoxycarbonyl)phenyl)-3-fluoropyridin-2-yl)piperidine-1-carboxylate (290 mg, 0.630 mmol) in dichloromethane (6.0 mL) at 0° C. was added trifluoroacetic acid (3.0 mL) dropwise. The reaction mixture was warmed to room temperature and stirred for 2 hours. Upon completion, the mixture was concentrated under reduced pressure. The crude residue was purified by reversed-phase flash chromatography (conditions: column: C18 silica gel; mobile phase A: water (10 mM NH₄HCO₃), mobile phase B: acetonitrile; Gradient (B %): 0% to 100% in 25 min; detector, UV 254 nm) to afford 4-(5-fluoro-6-(piperidin-2-yl)pyridin-3-yl)benzoic acid (75.0 mg, 39%) as a white solid. LCMS (ESI) [M+H]⁺: 301.1

### Step 4: 4-(5-fluoro-6-(1-((5-fluoro-6-(1-(trifluoromethyl)cyclopropyl)pyridin-3-yl)carbamoyl)piperidin-2-yl)pyridin-3-yl)benzoic acid

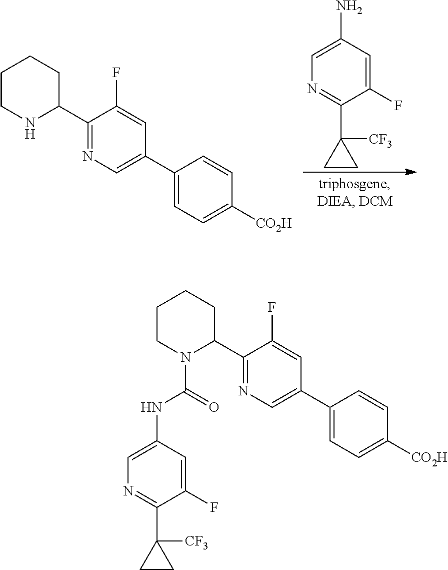 [SMILES: O=C(O)c1ccc(-c2cnc(C3CCCCN3)c(F)c2)cc1 ; Nc1cnc(C2(C(F)(F)F)CC2)c(F)c1 ; O=C(O)c1ccc(-c2cnc(C3CCCCN3C(=O)Nc3cnc(C4(C(F)(F)F)CC4)c(F)c3)c(F)c2)cc1]

To a mixture of 5-fluoro-6-(1-(trifluoromethyl)cyclopropyl)pyridin-3-amine (48.0 mg, 0.210 mmol) and diisopropylethylamine (140 mg, 1.08 mmol) in dichloromethane (3.0 mL) was added triphosgene (26 mg, 86.0 μmol) dropwise at 0° C. under nitrogen atmosphere. After 30 minutes, the mixture was warmed to room temperature and 4-(5-fluoro-6-(piperidin-2-yl)pyridin-3-yl)benzoic acid (65.0 mg, 0.210 mmol) was added dropwise. After 2 hours, the reaction was cooled in an ice bath and quenched with ice water. The mixture was extracted with ethyl acetate (3×10 mL). The combined organic layers were dried over anhydrous sodium sulfate, filtered, and concentrated under reduced pressure. The crude residue was purified by reversed-phase flash chromatography (conditions: column: C18 silica gel; mobile phase A: water (0.1% formic acid), mobile phase B: acetonitrile; Gradient (B %): 0% to 100% over 25 min; detector, UV 254 nm) to afford 4-(5-fluoro-6-(1-((5-fluoro-6-(1-(trifluoromethyl)cyclopropyl)pyridin-3-yl)carbamoyl)piperidin-2-yl)pyridin-3-yl)benzoic acid (60 mg, 51%) as a yellow solid. LCMS (ESI) [M+H]⁺: 547.1

### Step 5: (R)-4-(5-fluoro-6-(1-((5-fluoro-6-(1-(trifluoromethyl)cyclopropyl)pyridin-3-yl)carbamoyl)piperidin-2-yl)pyridin-3-yl)benzoic acid and (S)-4-(5-fluoro-6-(1-((5-fluoro-6-(1-(trifluoromethyl)cyclopropyl)pyridin-3-yl)carbamoyl)piperidin-2-yl)pyridin-3-yl)benzoic acid

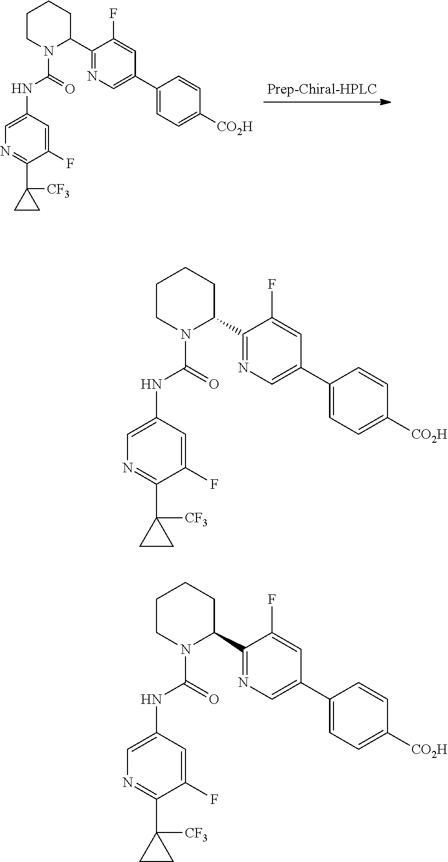

4-(5-fluoro-6-(1-((5-fluoro-6-(1-(trifluoromethyl)cyclopropyl)pyridin-3-yl)carbamoyl)piperidin-2-yl)pyridin-3-yl)benzoic acid (60 mg) was resolved by preparative chiral HPLC (conditions: column: CHIRALPAK ID, 3*25 cm, 5 μm; mobile phase A: hexane (0.1% formic acid); mobile phase B: ethanol; flow rate: 40 mL/min; gradient (B %): isocratic 25%; UV wavelength: 220/254 nm; RT1 (min): 6.5; RT2 (min): 11.3) to afford first peak (Compound 159a; Method AR, Peak 1, 0.77 min) (R)-4-(5-fluoro-6-(1-((5-fluoro-6-(1-(trifluoromethyl)cyclopropyl)pyridin-3-yl)carbamoyl)piperidin-2-yl)pyridin-3-yl)benzoic acid (20.1 mg, 34%) as a white solid and second peak (Compound 159b; Method AR, Peak 2, 1.57 min) (S)-4-(5-fluoro-6-(1-((5-fluoro-6-(1-(trifluoromethyl)cyclopropyl)pyridin-3-yl)carbamoyl)piperidin-2-yl)pyridin-3-yl)benz g 4 (19.1 mg, 32%) as a white solid.

| Cmpd # | Characterization Data |
|---|---|
| 159a | LCMS (ESI) [M + H]⁺: 547.3 |
| | ¹H NMR (400 MHz, DMSO-d₆) δ 9.14 (s, 1H), 8.78 (t, J = 1.6 Hz, 1H), 8.48 (bs, 1H), 8.12 (dd, |
| | J = 11.9, 1.9 Hz, 1H), 8.09-8.00 (m, 2H), 7.94-7.85 (m, 3H), 5.76 (d, J = 5.8 Hz, 1H), 4.14- |
| | 4.05 (m, 1H), 3.71-3.59 (m, 1H), 2.23-2.13 (m, 1H), 2.02-1.87 (m, 1H), 1.84-1.73 (m, 1H), |
| | 1.68-1.52 (m, 2H), 1.47-1.33 (m, 3H), 1.26-1.11 (m, 2H). |
| 159b | LCMS (ESI) [M + H]⁺: 547.3 |
| | ¹H NMR (400 MHz, DMSO-d₆) δ 9.14 (s, 1H), 8.80-8.75 (m, 1H), 8.51-8.46 (m, 1H), 8.12 (dd, |
| | J = 11.9, 1.9 Hz, 1H), 8.06-8.00 (m, 2H), 7.94-7.85 (m, 3H), 5.76 (d, J = 5.8 Hz, 1H), 4.15- |
| | 4.02 (m, 1H), 3.73-3.57 (m, 1H), 2.24-2.13 (m, 1H), 2.06-1.88 (m, 1H), 1.84-1.72 (m, 1H), |
| | 1.69-1.49 (m, 2H), 1.50-1.33 (m, 3H), 1.26-1.07 (m, 2H). |

### Example 161: 4-(5-fluoro-6-((2S,3R)-1-((5-fluoro-6-(1-(trifluoromethyl)cyclopropyl)pyridin-3-yl)carbamoyl)-3-methylpyrrolidin-2-yl)pyridin-3-yl)benzoic acid, 4-(5-fluoro-6-((2R,3R)-1-((5-fluoro-6-(1-(trifluoromethyl)cyclopropyl)pyridin-3-yl)carbamoyl)-3-methylpyrrolidin-2-yl)pyridin-3-yl)benzoic acid, 4-(5-fluoro-6-((2S,3S)-1-((5-fluoro-6-(1-(trifluoromethyl)cyclopropyl)pyridin-3-yl)carbamoyl)-3-methylpyrrolidin-2-yl)pyridin-3-yl)benzoic acid, and 4-(5-fluoro-6-((2R,3S)-1-((5-fluoro-6-(1-(trifluoromethyl)cyclopropyl)pyridin-3-yl)carbamoyl)-3-methylpyrrolidin-2-yl)pyridin-3-yl)benzoic acid

### Step 1: 1-(tert-butoxycarbonyl)-3-methyl-1H-pyrrole-2-carboxylic acid

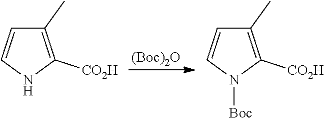

To a mixture of 3-methyl-1H-pyrrole-2-carboxylic acid (6.00 g, 47.9 mmol), di-tert-butyl dicarbonate (20.9 g, 95.9 mmol), and triethylamine (14.5 g, 144 mmol) in dichloromethane (60.0 mL) at room temperature was added DMAP (0.500 g, 4.79 mmol). The resultant mixture was stirred for 2 hours. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford 1-(tert-butoxycarbonyl)-3-methyl-1H-pyrrole-2-carboxylic acid (7.50 g, 69%) as a yellow solid. LCMS (ESI) [M+H]⁺: 226.1

### Step 2: 1-(tert-butoxycarbonyl)-3-methylpyrrolidine-2-carboxylic acid

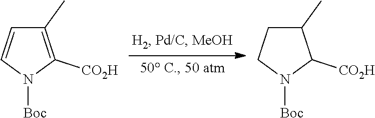 [SMILES: Cc1ccn(C(=O)OC(C)(C)C)c1C(=O)O ; CC1CCN(C(=O)OC(C)(C)C)C1C(=O)O]

A solution of 1-(tert-butoxycarbonyl)-3-methyl-1H-pyrrole-2-carboxylic acid (3.00 g, 13.3 mmol) and 10% palladium on carbon (900 mg) in methanol (120 mL) was heated to 50° C. under a hydrogen atmosphere at 50 psi for 24 hours. Upon completion, the hydrogen atmosphere was purged with nitrogen gas. The mixture was cooled to room temperature and filtered through celite. The filter cake was washed with methanol (3×30 mL). The filtrate was concentrated under reduced pressure to afford 1-(tert-butoxycarbonyl)-3-methylpyrrolidine-2-carboxylic acid (1.00 g, crude) as a brown oil, which was used directly in the next step without further purification. LCMS (ESI) [M+H]⁺: 230.0

### Step 3: tert-butyl 2-(3-fluoro-5-(4-(methoxycarbonyl)phenyl)pyridin-2-yl)-3-methylpyrrolidine-1-carboxylate

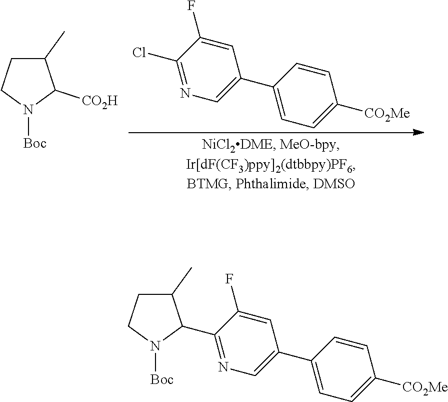 [SMILES: CC1CCN(C(=O)OC(C)(C)C)C1C(=O)O ; COC(=O)c1ccc(-c2cnc(Cl)c(F)c2)cc1 ; COC(=O)c1ccc(-c2cnc(C3C(C)CCN3C(=O)OC(C)(C)C)c(F)c2)cc1]

A solution of 1-(tert-butoxycarbonyl)-3-methylpyrrolidine-2-carboxylic acid (1.00 g, 4.36 mmol), methyl 4-(6-chloro-5-fluoropyridin-3-yl)benzoate (1.30 g, 5.23 mmol), phthalimide (600 mg, 4.36 mmol), 4,4′-dimethoxy-2,2′-bipyridine (100 mg, 0.520 mmol), dichloronickel·1,2-dimethoxyethane (100 mg, 0.430 mmol), Ir[dF(CF₃)ppy]₂(dtbpy))PF₆ (100 mg, 0.080 mmol), and N″-tert-butyl-N,N,N′,N′-tetramethylguanidine (1.10 g, 6.54 mmol) in DMSO (50.0 mL) at room temperature was stirred for 24 hours under 450 nm blue LED irradiation. Upon completion, the mixture was diluted with water (50 mL) and extracted with ethyl acetate (3×50 mL). The combined layers were washed with water (3×50 mL), dried over anhydrous sodium sulfate, filtered, and concentrated under reduced pressure. The crude residue was purified by silica gel column chromatography, eluted with petroleum ether/ethyl acetate (3:1) to afford tert-butyl 2-(3-fluoro-5-(4-(methoxycarbonyl)phenyl)pyridin-2-yl)-3-methylpyrrolidine-1-carboxylate (400 mg, 22%) as a yellow oil. LCMS (ESI) [M+H]⁺: 415.2

### Step 4: methyl 4-(5-fluoro-6-(3-methylpyrrolidin-2-yl)pyridin-3-yl)benzoate

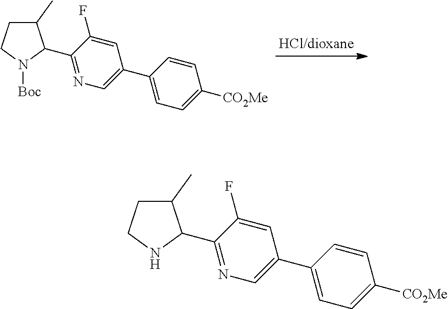 [SMILES: COC(=O)c1ccc(-c2cnc(C3C(C)CCN3C(=O)OC(C)(C)C)c(F)c2)cc1 ; COC(=O)c1ccc(-c2cnc(C3NCCC3C)c(F)c2)cc1]

To a solution of tert-butyl 2-(3-fluoro-5-(4-(methoxycarbonyl)phenyl)pyridin-2-yl)-3-methylpyrrolidine-1-carboxylate (400 mg, 0.960 mmol) in 1,4-dioxane (1.0 mL) was added a solution of HCl (5.0 mL, 10.0 mmol, 2 M in dioxane), and the resultant mixture was stirred at room temperature for 1 hour. Upon completion, the mixture was concentrated under reduced pressure to afford methyl 4-(5-fluoro-6-(3-methylpyrrolidin-2-yl)pyridin-3-yl)benzoate (380 mg, crude) as a yellow solid, which was used directly in the next step without further purification. LCMS (ESI) [M+H]⁺: 315.1

### Step 5: methyl 4-(5-fluoro-6-(1-((5-fluoro-6-(1-(trifluoromethyl)cyclopropyl)pyridin-3-yl)carbamoyl)-3-methylpyrrolidin-2-yl)pyridin-3-yl)benzoate

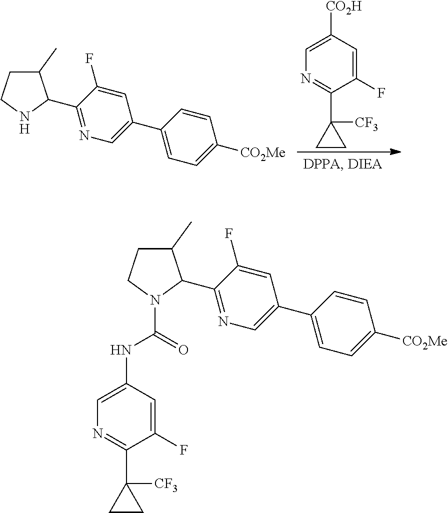 [SMILES: COC(=O)c1ccc(-c2cnc(C3NCCC3C)c(F)c2)cc1 ; O=C(O)c1cnc(C2(C(F)(F)F)CC2)c(F)c1 ; COC(=O)c1ccc(-c2cnc(C3C(C)CCN3C(=O)Nc3cnc(C4(C(F)(F)F)CC4)c(F)c3)c(F)c2)cc1]

A solution of 5-fluoro-6-(1-(trifluoromethyl)cyclopropyl)nicotinic acid (118 mg, 0.477 mmol), DPPA (142 mg, 0.515 mmol) and N, N-diisopropylethylamine (61.3 mg, 0.461 mmol) in toluene (2.0 mL) at room temperature. After 30 minutes, methyl 4-(5-fluoro-6-(3-methylpyrrolidin-2-yl)pyridin-3-yl)benzoate (150 mg, 0.477 mmol) was added and the reaction was heated at 80° C. for 2 hours. Upon completion, the mixture was cooled to room temperature and concentrated under reduced pressure. The crude product was purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford methyl 4-(5-fluoro-6-(1-((5-fluoro-6-(1-(trifluoromethyl)cyclopropyl)pyridin-3-yl)carbamoyl)-3-methylpyrrolidin-2-yl)pyridin-3-yl)benzoate (130 mg, 49%) as a colorless oil. LCMS (ESI) [M+H]⁺: 561.2

### Step 6: 4-(5-fluoro-6-(1-((5-fluoro-6-(1-(trifluoromethyl)cyclopropyl)pyridin-3-yl)carbamoyl)-3-methylpyrrolidin-2-yl)pyridin-3-yl)benzoic acid

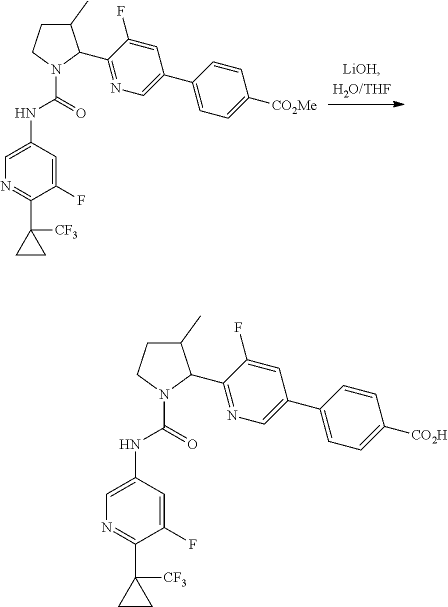 [SMILES: COC(=O)c1ccc(-c2cnc(C3C(C)CCN3C(=O)Nc3cnc(C4(C(F)(F)F)CC4)c(F)c3)c(F)c2)cc1 ; CC1CCN(C(=O)Nc2cnc(C3(C(F)(F)F)CC3)c(F)c2)C1c1ncc(-c2ccc(C(=O)O)cc2)cc1F]

A solution of methyl 4-(5-fluoro-6-(1-((5-fluoro-6-(1-(trifluoromethyl)cyclopropyl)pyridin-3-yl)carbamoyl)-3-methylpyrrolidin-2-yl)pyridin-3-yl)benzoate (130 mg, 0.230 mmol) and lithium hydroxide monohydrate (38.6 mg, 0.920 mmol) in THF (2.0 mL) and water (0.30 mL) was stirred at 50° C. for 2 hours. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford 4-(5-fluoro-6-(1-((5-fluoro-6-(1-(trifluoromethyl)cyclopropyl)pyridin-3-yl)carbamoyl)-3-methylpyrrolidin-2-yl)pyridin-3-yl)benzoic acid (68 mg, 54% yield) as a white solid. LCMS (ESI) [M+H]⁺: 547.2

### Step 6: 4-(5-fluoro-6-((2S,3R)-1-((5-fluoro-6-(1-(trifluoromethyl)cyclopropyl)pyridin-3-yl)carbamoyl)-3-methylpyrrolidin-2-yl)pyridin-3-yl)benzoic acid, 4-(5-fluoro-6-((2R,3R)-1-((5-fluoro-6-(1-(trifluoromethyl)cyclopropyl)pyridin-3-yl)carbamoyl)-3-methylpyrrolidin-2-yl)pyridin-3-yl)benzoic acid, 4-(5-fluoro-6-((2S,3S)-1-((5-fluoro-6-(1-(trifluoromethyl)cyclopropyl)pyridin-3-yl)carbamoyl)-3-methylpyrrolidin-2-yl)pyridin-3-yl)benzoic acid and 4-(5-fluoro-6-((2R,3S)-1-((5-fluoro-6-(1-(trifluoromethyl)cyclopropyl)pyridin-3-yl)carbamoyl)-3-methylpyrrolidin-2-yl)pyridin-3-yl)benzoic acid

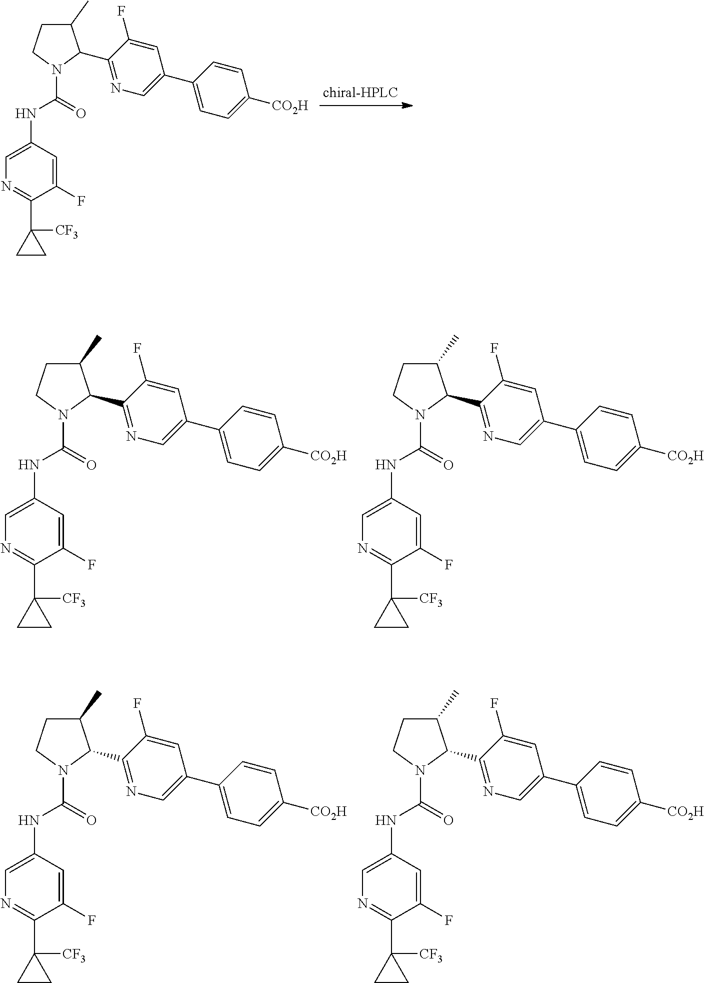 [SMILES: CC1CCN(C(=O)Nc2cnc(C3(C(F)(F)F)CC3)c(F)c2)C1c1ncc(-c2ccc(C(=O)O)cc2)cc1F ; C[C@@H]1CCN(C(=O)Nc2cnc(C3(C(F)(F)F)CC3)c(F)c2)[C@@H]1c1ncc(-c2ccc(C(=O)O)cc2)cc1F ; C[C@H]1CCN(C(=O)Nc2cnc(C3(C(F)(F)F)CC3)c(F)c2)[C@@H]1c1ncc(-c2ccc(C(=O)O)cc2)cc1F ; C[C@@H]1CCN(C(=O)Nc2cnc(C3(C(F)(F)F)CC3)c(F)c2)[C@H]1c1ncc(-c2ccc(C(=O)O)cc2)cc1F ; C[C@H]1CCN(C(=O)Nc2cnc(C3(C(F)(F)F)CC3)c(F)c2)[C@H]1c1ncc(-c2ccc(C(=O)O)cc2)cc1F]

4-(5-Fluoro-6-(1-((5-fluoro-6-(1-(trifluoromethyl)cyclopropyl)pyridin-3-yl)carbamoyl)-3-methylpyrrolidin-2-yl)pyridin-3-yl)benzoic acid (67.0 mg) was resolved by prep chiral HPLC (conditions: column: CHIRAL ART Cellulose-SC, 3*25 cm, 5 μm; mobile phase A: hexane (0.1% formic acid); mobile phase B: ethanol; flow rate: 40 mL/min; gradient (B %): isocratic 15% B; detector: 220/254 nm; RT1 (min): 8.9; RT2 (min): 10.4; RT3 (min): 15.3) to afford (Compound 161a; Method W, Peak 1, 0.76 min) 4-(5-fluoro-6-((2S,3R)-1-((5-fluoro-6-(1-(trifluoromethyl)cyclopropyl)pyridin-3-yl)carbamoyl)-3-methylpyrrolidin-2-yl)pyridin-3-yl)benzoic acid (1^{st} peak, 9.7 mg, 11%) as a white solid, (Compound 161b; Method W, Peak 4, 1.46 min) 4-(5-fluoro-6-((2R,3S)-1-((5-fluoro-6-(1-(trifluoromethyl)cyclopropyl)pyridin-3-yl)carbamoyl)-3-methylpyrrolidin-2-yl)pyridin-3-yl)benzoic acid (3^{rd} peak, 11.3 mg, 17%) as a white solid, and a mixture (2^{nd} peak, 32.0 mg, 40%). The mixture was further resolved by prep chiral HPLC (conditions: column: Chiral NX(2)5μ 3*25 cm, 5 μm; mobile phase A: hexane (0.1% formic acid); mobile phase B: ethanol; flow rate: 40 mL/min; gradient (B %): isocratic 50% B; detector: 220/254 nm; RT1 (min): 6.8; RT2 (min): 12.5) to afford (Compound 161c; Method W, Peak 2, 0.88 min) 4-(5-fluoro-6-((2R,3R)-1-((5-fluoro-6-(1-(trifluoromethyl)cyclopropyl)pyridin-3-yl)carbamoyl)-3-methylpyrrolidin-2-yl)pyridin-3-yl)benzoic acid (1^{st} peak, 10.1 mg, 15.1%) as a white solid and (Compound 161d; Method W, Peak 3, 1.14 min) 4-(5-fluoro-6-((2S,3S)-1-((5-fluoro-6-(1-(trifluoromethyl)cyclopropyl)pyridin-3-yl)carbamoyl)-3-methylpyrrolidin-2-yl)pyridin-3-yl)benzoic acid (2^{nd} peak, 8.6 mg, 12.8%) as a white solid.

| Cmpd # | Characterization Data |
|---|---|
| 161a | LCMS (ESI) [M + H]⁺: 547.2 |
| | ¹H NMR (400 MHz, DMSO-d₆) δ 8.89 (s, 1H), 8.70 (s, 1H), 8.46 (s, 1H), 8.23 (s, 1H), 8.06 |
| | (dd, J = 11.4, 1.9 Hz, 1H), 8.03-7.95 (m, 2H), 7.87 (dd, J = 12.8, 2.1 Hz, 1H), 7.62 (t, J = 7.8 |
| | Hz, 1H), 4.86 (d, J = 6.2 Hz, 1H), 3.82-3.70 (m, 2H), 2.39-2.30 (m, 1H), 2.26-2.17 (m, |
| | 1H), 1.79-1.67 (m, 1H), 1.41-1.32 (m, 2H), 1.20-1.04 (m, 5H). |
| 161c | LCMS (ESI) [M + H]⁺: 547.2 |
| | ¹H NMR (400 MHz, DMSO-d₆) δ 8.91 (s, 1H), 8.74 (s, 1H), 8.46 (s, 1H), 8.08 (d, J = 11.4 Hz, |
| | 1H), 8.03 (d, J = 8.1 Hz, 2H), 7.93-7.81 (m, 3H), 4.86 (d, J = 6.2 Hz, 1H), 3.80-3.73 (m, |
| | 2H), 2.38-2.31 (m, 1H), 2.24-2.19 (m, 1H), 1.79-1.72 (m, 1H), 1.41-1.32 (m, 2H), 1.22- |
| | 1.07 (m, 5H). |
| 161d | LCMS (ESI) [M + H]⁺: 547.2 |
| | ¹H NMR (400 MHz, DMSO-d₆) δ 8.89 (s, 1H), 8.70 (s, 1H), 8.46 (s, 1H), 8.23 (s, 1H), 8.07 |
| | (dd, J = 11.4, 1.9 Hz, 1H), 7.99 (d, J = 7.8 Hz, 2H), 7.87 (dd, J = 12.8, 2.1 Hz, 1H), 7.63 (t, J = |
| | 7.7 Hz, 1H), 4.86 (d, J = 6.1 Hz, 1H), 3.82-3.73 (m, 2H), 2.38-2.29 (m, 1H), 2.26-2.17 |
| | (m, 1H), 1.80-1.71 (m, 1H), 1.39-1.32 (m, 2H), 1.20-1.07 (m, 5H). |
| 161b | LCMS (ESI) [M + H]⁺: 547.2 |
| | ¹H NMR (400 MHz, DMSO-d₆) δ 8.90 (s, 1H), 8.75 (s, 1H), 8.46 (s, 1H), 8.08 (dd, J = 11.5, |
| | 1.9 Hz, 1H), 8.03 (d, J = 8.2 Hz, 2H), 7.94-7.80 (m, 3H), 4.86 (d, J = 6.2 Hz, 1H), 3.82- |
| | 3.73 (m, 2H), 2.34 (p, J = 6.8 Hz, 1H), 2.27-2.11 (m, 1H), 1.80-1.69 (m, 1H), 1.41-1.31 |
| | (m, 2H), 1.18-1.08 (m, 5H). |

### Example 162: (S)-2-(5-(1H-benzo[d][1,2,3]triazol-5-yl)-3-fluoropyridin-2-yl)-N-(2,2-difluorobenzo[d][1,3]dioxol-5-yl)pyrrolidine-1-carboxamide and (R)-2-(5-(1H-benzo[d][1,2,3]triazol-5-yl)-3-fluoropyridin-2-yl)-N-(2,2-difluorobenzo[d][1,3]dioxol-5-yl)pyrrolidine-1-carboxamide

### Step 1: tert-butyl 5-bromo-1H-benzo[d][1,2,3]triazole-1-carboxylate

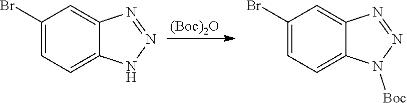 [SMILES: "(Boc)2O" ; Brc1ccc2[nH]nnc2c1 ; CC(C)(C)OC(=O)n1nnc2cc(Br)ccc21]

To 5-bromo-1H-1,2,3-benzotriazole (400 mg, 2.02 mmol), triethylamine (307 mg, 3.03 mmol), and di-tert-butyl dicarbonate (529 mg, 2.42 mmol) in dichloromethane (5.0 mL) at room temperature was added DMAP (12.3 mg, 0.101 mmol), and the resultant solution was stirred for 1 hour. Upon completion, the mixture was concentrated under reduced pressure. The crude residue was purified by silica gel column chromatography, eluted with petroleum ether/ethyl acetate (5:1), to afford tert-butyl 5-bromo-1H-benzo[d][1,2,3]triazole-1-carboxylate (300 mg, 50%) as a white solid. LCMS (ESI) [M+H]⁺: 298.0

### Step 2: (5-fluoro-6-(pyrrolidin-2-yl)pyridin-3-yl)boronic acid hydrochloride

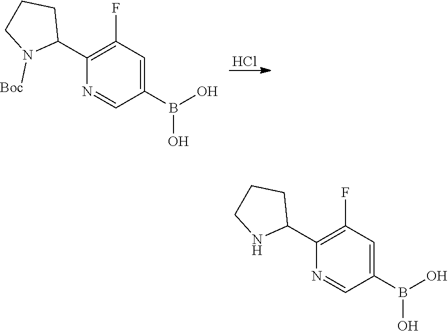 [SMILES: CC(C)(C)OC(=O)N1CCCC1c1ncc(B(O)O)cc1F ; Cl ; OB(O)c1cnc(C2CCCN2)c(F)c1]

To a solution of (6-(1-(tert-butoxycarbonyl)pyrrolidin-2-yl)-5-fluoropyridin-3-yl)boronic acid (200 mg, 0.645 mmol) in dichloromethane (5.0 mL) was added a solution of HCl in 1,4-dioxane (1.0 mL, 2.0 mmol, 2.0 M), and the resultant solution was stirred at room temperature for 1 hour. Upon completion, the mixture was concentrated under reduced pressure to afford (5-fluoro-6-(pyrrolidin-2-yl)pyridin-3-yl)boronic acid hydrochloride (120 mg, crude) as a yellow oil, which was used directly in the next step without further purification. LCMS (ESI) [M+H]⁺: 211.1

### Step 3: tert-butyl 5-(5-fluoro-6-(pyrrolidin-2-yl)pyridin-3-yl)-1H-benzo[d][1,2,3]triazole-1-carboxylate

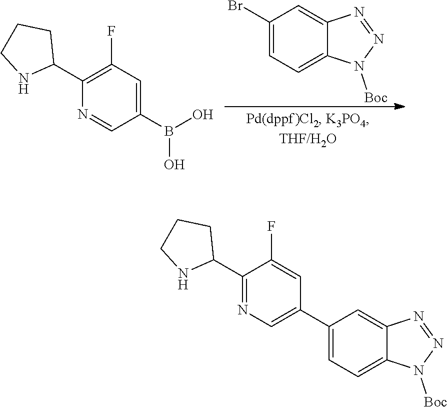 [SMILES: OB(O)c1cnc(C2CCCN2)c(F)c1 ; CC(C)(C)OC(=O)n1nnc2cc(Br)ccc21 ; CC(C)(C)OC(=O)n1nnc2cc(-c3cnc(C4CCCN4)c(F)c3)ccc21]

A solution of (5-fluoro-6-(pyrrolidin-2-yl)pyridin-3-yl)boronic acid hydrochloride (100 mg, 0.406 mmol), tert-butyl 5-bromo-1H-benzo[d][1,2,3]triazole-1-carboxylate (170 mg, 0.571 mmol), Pd(dppf)Cl₂ (34.8 mg, 0.048 mmol) and K₃PO₄ (303 mg, 1.43 mmol) in THF (1.0 mL) and water (0.10 mL) was stirred at 80° C. for 1 hour. Upon completion, the resultant mixture was concentrated under reduced pressure. The crude residue was purified by silica gel column chromatography, eluted with petroleum ether/ethyl acetate (1:1), to afford tert-butyl 5-(5-fluoro-6-(pyrrolidin-2-yl)pyridin-3-yl)-1H-benzo[d][1,2,3]triazole-1-carboxylate (80.0 mg, 44%) as a brown solid. LCMS (ESI) [M+H]⁺: 384.1

### Step 4: 5-(5-fluoro-6-(pyrrolidin-2-yl)pyridin-3-yl)-1H-benzo[d][1,2,3]triazole

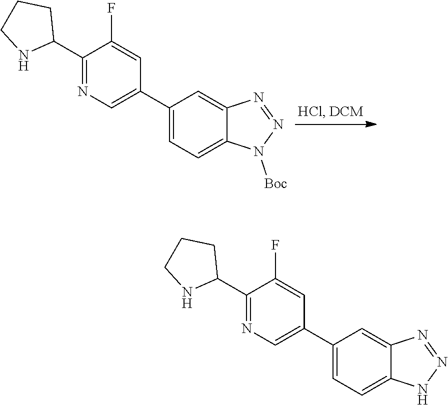 [SMILES: CC(C)(C)OC(=O)n1nnc2cc(-c3cnc(C4CCCN4)c(F)c3)ccc21 ; Fc1cc(-c2ccc3[nH]nnc3c2)cnc1C1CCCN1]

To a solution of tert-butyl 5-(5-fluoro-6-(pyrrolidin-2-yl)pyridin-3-yl)-1H-benzo[d][1,2,3]triazole-1-carboxylate (80 mg, 0.209 mmol) in dichloromethane (5.0 mL) was added a solution of HCl (1.0 mL, 2 mmol, 2.0 M in 1,4-dioxane), and the resultant mixture was stirred at room temperature for 1 hour. Upon completion, the mixture was concentrated under reduced pressure to afford 5-(5-fluoro-6-(pyrrolidin-2-yl)pyridin-3-yl)-1H-benzo[d][1,2,3]triazole (50.0 mg, crude) as a yellow oil, which was used directly in the next step without further purification. LCMS (ESI) [M+H]⁺: 284.1

### Step 5: 2-(5-(1H-benzo[d][1,2,3]triazol-5-yl)-3-fluoropyridin-2-yl)-N-(2,2-difluorobenzo[d][1,3]dioxol-5-yl)pyrrolidine-1-carboxamide

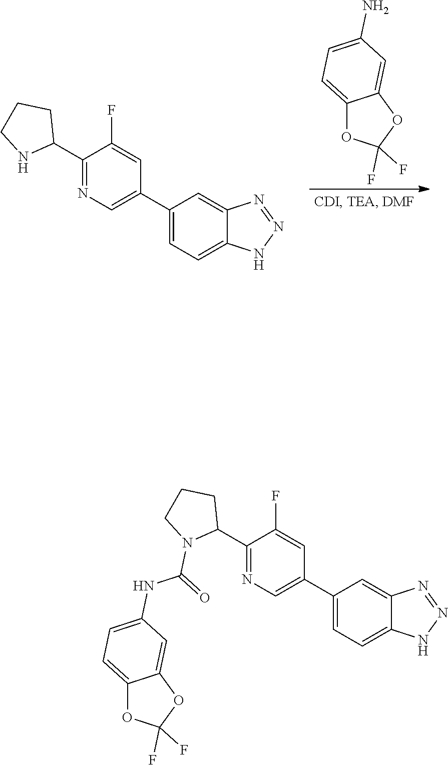 [SMILES: Fc1cc(-c2ccc3[nH]nnc3c2)cnc1C1CCCN1 ; Nc1ccc2c(c1)OC(F)(F)O2 ; O=C(Nc1ccc2c(c1)OC(F)(F)O2)N1CCCC1c1ncc(-c2ccc3[nH]nnc3c2)cc1F]

A solution of 5-(5-fluoro-6-(pyrrolidin-2-yl)pyridin-3-yl)-1H-benzo[d][1,2,3]triazole (50.0 mg, 0.176 mmol), 2,2-difluoro-1,3-benzodioxol-5-amine (61.1 mg, 0.352 mmol), carbonyldiimidazole (143 mg, 0.880 mmol) and triethylamine (89.3 mg, 0.880 mmol) in DMF (1.0 mL) was stirred at 40° C. for 1 hour. Upon completion, the mixture was directly purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford 2-(5-(1H-benzo[d][1,2,3]triazol-5-yl)-3-fluoropyridin-2-yl)-N-(2,2-difluorobenzo[d][1,3]dioxol-5-yl)pyrrolidine-1-carboxamide (35 mg, 41%) as a white solid. LCMS (ESI) [M+H]⁺: 483.1

### Step 6: (S)-2-(5-(1H-benzo[d][1,2,3]triazol-5-yl)-3-fluoropyridin-2-yl)-N-(2,2-difluorobenzo[d][1,3]dioxol-5-yl)pyrrolidine-1-carboxamide and (R)-2-(5-(1H-benzo[d][1,2,3]triazol-5-yl)-3-fluoropyridin-2-yl)-N-(2,2-difluorobenzo[d][1,3]dioxol-5-yl)pyrrolidine-1-carboxamide

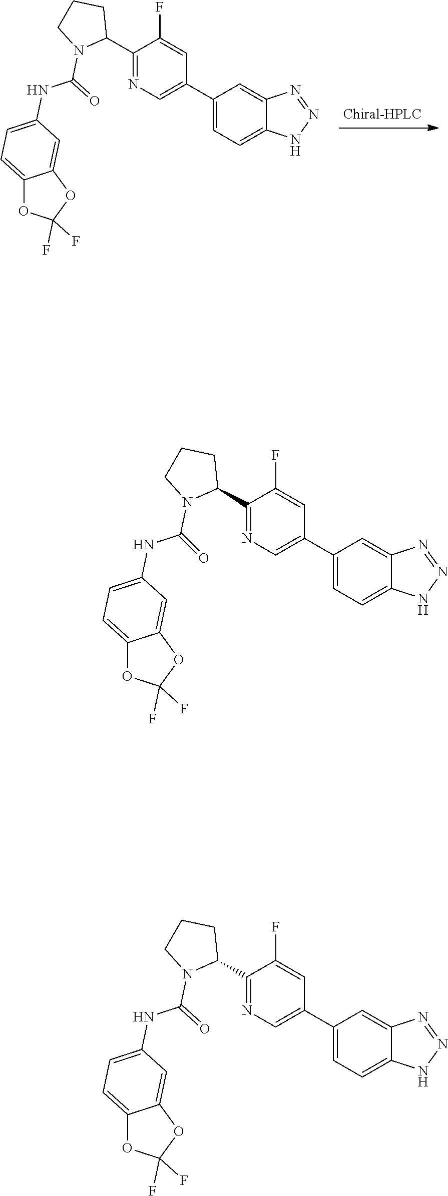 [SMILES: O=C(Nc1ccc2c(c1)OC(F)(F)O2)N1CCCC1c1ncc(-c2ccc3[nH]nnc3c2)cc1F ; O=C(Nc1ccc2c(c1)OC(F)(F)O2)N1CCC[C@H]1c1ncc(-c2ccc3[nH]nnc3c2)cc1F ; O=C(Nc1ccc2c(c1)OC(F)(F)O2)N1CCC[C@@H]1c1ncc(-c2ccc3[nH]nnc3c2)cc1F]

2-(5-(1H-Benzo[d][1,2,3]triazol-5-yl)-3-fluoropyridin-2-yl)-N-(2,2-difluorobenzo[d][1,3]dioxol-5-yl)pyrrolidine-1-carboxamide (35 mg) was resolved by preparative chiral HPLC (conditions: column: CHIRAL ART Cellulose-SB, 3*25 cm, 5 μm; mobile phase A: hexane (0.1% formic acid); mobile phase B: ethanol; Flow rate: 40 mL/min; gradient (B %): isocratic 15% B; detector: 220/254 nm; RT1 (min): 15.4; RT2 (min): 18.5) to afford (Compound 162a; Method BC, Peak 1, 1.85 min) (S)-2-(5-(1H-benzo[d][1,2,3]triazol-5-yl)-3-fluoropyridin-2-yl)-N-(2,2-difluorobenzo[d][1,3]dioxol-5-yl)pyrrolidine-1-carboxamide (1^{st} peak, 10.0 mg, 29%) and (Compound 162b; Method BC, Peak 2, 2.26 min) (R)-2-(5-(1H-benzo[d][1,2,3]triazol-5-yl)-3-fluoropyridin-2-yl)-N-(2,2-difluorobenzo[d][1,3]dioxol-5-yl)pyrrolidine-1-carboxamide (2^{nd} peak, 13.0 mg, 37%).

| Cmpd # | Characterization Data |
|---|---|
| 162a | LCMS (ESI) [M + H]⁺: 483.2 |
| | ¹H NMR (400 MHz, methanol-d₄) δ 8.67 (s, 1H), 8.16 (s, 1H), 8.04-7.89 (m, 2H), 7.79 (dd, |
| | J = 8.7, 1.6 Hz, 1H), 7.36 (d, J = 1.9 Hz, 1H), 7.11-6.98 (m, 2H), 5.51-5.40 (m, 1H), 3.93- |
| | 3.81 (m, 1H), 3.76-3.65 (m, 1H), 2.54-2.42 (m, 1H), 2.25-2.02 (m, 3H). |
| 162b | LCMS (ESI) [M + H]⁺: 483.2 |
| | ¹H NMR (400 MHz, methanol-d₄) δ 8.67 (s, 1H), 8.28-7.89 (m, 3H), 7.84-7.73 (m, 1H), |
| | 7.36 (d, J = 1.9 Hz, 1H), 7.12-6.98 (m, 2H), 5.53-5.39 (m, 1H), 3.90-3.81 (m, 1H), 3.75- |
| | 3.64 (m, 1H), 2.55-2.37 (m, 1H), 2.25-2.03 (m, 3H). |

### Example 163: (S)-2-(5-(1H-benzo[d][1,2,3]triazol-5-yl)-3-fluoropyridin-2-yl)-N-(4-(1-(trifluoromethyl)cyclopropyl)phenyl)pyrrolidine-1-carboxamide and (R)-2-(5-(1H-benzo[d][1,2,3]triazol-5-yl)-3-fluoropyridin-2-yl)-N-(4-(1-(trifluoromethyl)cyclopropyl)phenyl)pyrrolidine-1-carboxamide

### Step 1: 2-(5-chloro-3-fluoropyridin-2-yl)-N-(4-(1-(trifluoromethyl)cyclopropyl)phenyl)pyrrolidine-1-carboxamide

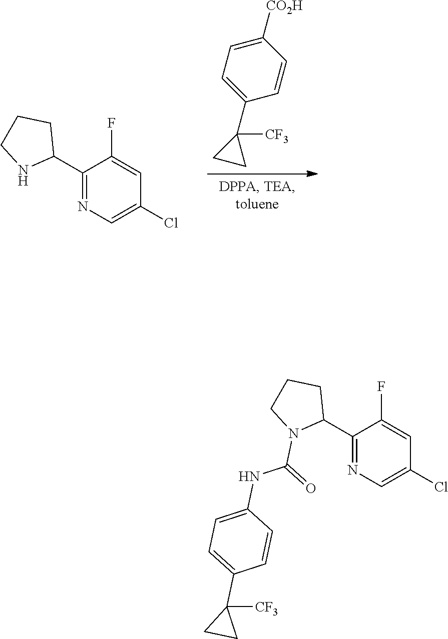 [SMILES: Fc1cc(Cl)cnc1C1CCCN1 ; O=C(O)c1ccc(C2(C(F)(F)F)CC2)cc1 ; O=C(Nc1ccc(C2(C(F)(F)F)CC2)cc1)N1CCCC1c1ncc(Cl)cc1F]

A solution of 4-(1-(trifluoromethyl)cyclopropyl)benzoic acid (241 mg, 1.05 mmol), DPPA (384 mg, 1.40 mmol) and triethylamine (212 mg, 2.09 mmol) in toluene (2.0 mL) was stirred at room temperature. After 30 minutes, 5-chloro-3-fluoro-2-(pyrrolidin-2-yl)pyridine (140 mg, 0.698 mmol) was added, and the resultant mixture was stirred at 80° C. for 1 hour. Upon completion, the mixture was concentrated under reduced pressure. The crude residue was purified by silica gel column chromatography, eluted with petroleum ether/ethyl acetate (1:1) to afford 2-(5-chloro-3-fluoropyridin-2-yl)-N-(4-(1-(trifluoromethyl)cyclopropyl)phenyl)pyrrolidine-1-carboxamide (160 mg, 54%) as a pale-yellow oil. LCMS (ESI) [M+H]⁺: 428.1

### Step 2: 2-(5-(1H-benzo[d][1,2,3]triazol-5-yl)-3-fluoropyridin-2-yl)-N-(4-(1-(trifluoromethyl)cyclopropyl)phenyl)pyrrolidine-1-carboxamide

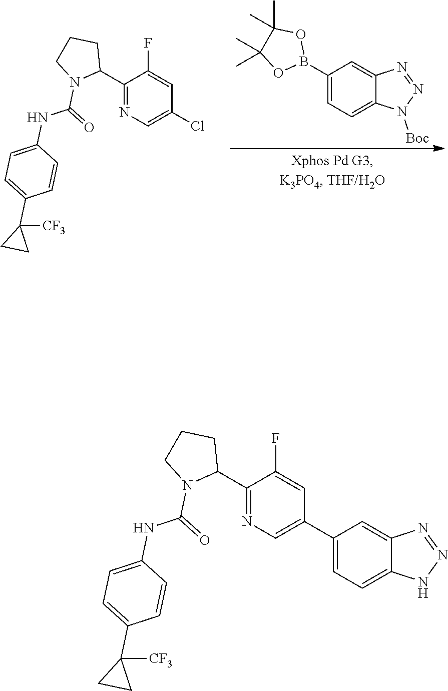 [SMILES: O=C(Nc1ccc(C2(C(F)(F)F)CC2)cc1)N1CCCC1c1ncc(Cl)cc1F ; CC(C)(C)OC(=O)n1nnc2cc(B3OC(C)(C)C(C)(C)O3)ccc21 ; O=C(Nc1ccc(C2(C(F)(F)F)CC2)cc1)N1CCCC1c1ncc(-c2ccc3[nH]nnc3c2)cc1F]

To a solution of 2-(5-chloro-3-fluoropyridin-2-yl)-N-(4-(1-(trifluoromethyl)cyclopropyl)phenyl)pyrrolidine-1-carboxamide (150 mg, 0.351 mmol) in THF (2.0 mL) and water (0.20 mL) was added tert-butyl 5-(4,4,5,5-tetramethyl-1,3,2-dioxaborolan-2-yl)-1,2,3-benzotriazole-1-carboxylate (182 mg, 0.526 mmol), K₃PO₄ (223 mg, 1.05 mmol), and XPhos Pd G3 (59.4 mg, 0.070 mmol), and the mixture was stirred at 80° C. for 2 hours. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford 2-(5-(1H-benzo[d][1,2,3]triazol-5-yl)-3-fluoropyridin-2-yl)-N-(4-(1-(trifluoromethyl)cyclopropyl)phenyl)pyrrolidine-1-carboxamide (37 mg, 21%) as a white solid. LCMS (ESI) [M+H]⁺: 511.2

### Step 3: (S)-2-(5-(1H-benzo[d][1,2,3]triazol-5-yl)-3-fluoropyridin-2-yl)-N-(4-(1-(trifluoromethyl)cyclopropyl)phenyl)pyrrolidine-1-carboxamide and (R)-2-(5-(1H-benzo[d][1,2,3]triazol-5-yl)-3-fluoropyridin-2-yl)-N-(4-(1-(trifluoromethyl)cyclopropyl)phenyl)pyrrolidine-1-carboxamide

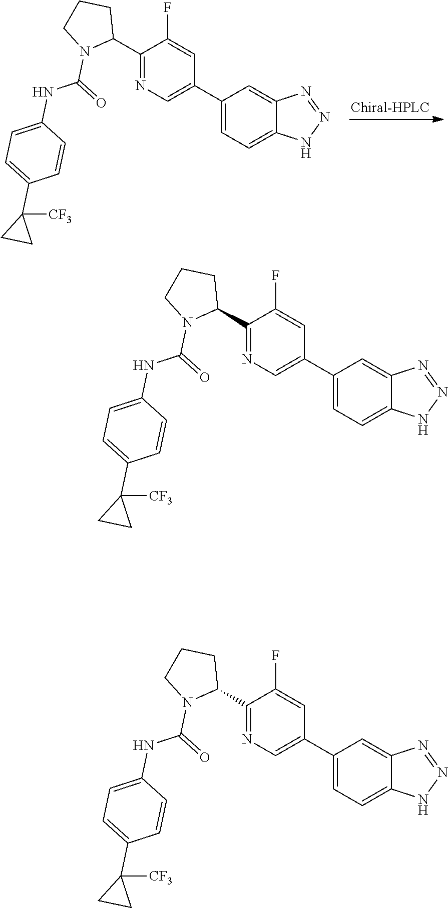 [SMILES: O=C(Nc1ccc(C2(C(F)(F)F)CC2)cc1)N1CCCC1c1ncc(-c2ccc3[nH]nnc3c2)cc1F ; O=C(Nc1ccc(C2(C(F)(F)F)CC2)cc1)N1CCC[C@H]1c1ncc(-c2ccc3[nH]nnc3c2)cc1F ; O=C(Nc1ccc(C2(C(F)(F)F)CC2)cc1)N1CCC[C@@H]1c1ncc(-c2ccc3[nH]nnc3c2)cc1F]

2-(5-(1H-Benzo[d][1,2,3]triazol-5-yl)-3-fluoropyridin-2-yl)-N-(4-(1-(trifluoromethyl)cyclopropyl)phenyl)pyrrolidine-1-carboxamide (37.0 mg) was resolved by preparative chiral HPLC (conditions: Column: CHIRAL ART Cellulose-SC, 3*25 cm, 5 μm; Mobile Phase A: Hexane (0.1% FA), Mobile Phase B: EtOH; Flow rate: 40 mL/min; Gradient (B %): isocratic 30% B; Wave Length: 220/254 nm; RT1 (min): 8.5; RT2 (min): 10.8) to afford (Compound 163a; Method AA, Peak 1, 0.99 min) (S)-2-(5-(1H-benzo[d][1,2,3]triazol-5-yl)-3-fluoropyridin-2-yl)-N-(4-(1-(trifluoromethyl)cyclopropyl)phenyl)pyrrolidine-1-carboxamide (1^{st} peak, 9.7 mg, 26%) as a white solid and (Compound 163b; Method AA, Peak 2, 1.50 min) (R)-2-(5-(1H-benzo[d][1,2,3]triazol-5-yl)-3-fluoropyridin-2-yl)-N-(4-(1-(trifluoromethyl)cyclopropyl)phenyl)pyrrolidine-1-carboxamide (2^{nd} peak, 9.6 mg, 26%) as a white solid.

| Cmpd # | Characterization Data |
|---|---|
| 163a | LCMS (ESI) [M + H]⁺: 511.2 |
| | ¹H NMR (400 MHz, DMSO-d₆) δ 15.86 (s, 1H), 8.77 (s, 1H), 8.35 (s, 1H), 8.30 (s, 1H), 8.11 |
| | (dd, J = 11.6, 1.9 Hz, 1H), 8.01 (d, J = 8.7 Hz, 1H), 7.83 (d, J = 8.7 Hz, 1H), 7.45 (d, J = 8.7 |
| | Hz, 2H), 7.26 (d, J = 8.6 Hz, 2H), 5.44-5.31 (m, 1H), 3.85-3.69 (m, 1H), 3.69-3.55 (m, |
| | 1H), 2.42-2.28 (m, 1H), 2.20-2.07 (m, 1H), 2.07-1.96 (m, 1H), 1.96-1.85 (m, 1H), 1.26 |
| | (d, J = 2.2 Hz, 2H), 1.02 (s, 2H). |
| 163b | LCMS (ESI) [M + H]⁺: 511.3 |
| | ¹H NMR (400 MHz, DMSO-d₆) δ 15.86 (s, 1H), 8.77 (d, J = 1.7 Hz, 1H), 8.35 (s, 1H), 8.30 (s, |
| | 1H), 8.11 (dd, J = 11.6, 1.9 Hz, 1H), 8.01 (d, J = 8.7 Hz, 1H), 7.83 (d, J = 8.6 Hz, 1H), 7.45 (d, |
| | J = 8.7 Hz, 2H), 7.26 (d, J = 8.5 Hz, 2H), 5.42-5.25 (m, 1H), 3.83-3.72 (m, 1H), 3.70-3.57 |
| | (m, 1H), 2.41-2.29 (m, 1H), 2.20-2.09 (m, 1H), 2.09-1.96 (m, 1H), 1.96-1.86 (m, 1H), |
| | 1.25 (dd, J = 6.8, 2.3 Hz, 2H), 1.02 (s, 2H). |

### Example 164: (S)-2-(5-(1H-benzo[d][1,2,3]triazol-5-yl)-3-fluoropyridin-2-yl)-N-(2,2-difluoro-[1,3]dioxolo[4,5-b]pyridin-6-yl)pyrrolidine-1-carboxamide and (R)-2-(5-(1H-benzo[d][1,2,3]triazol-5-yl)-3-fluoropyridin-2-yl)-N-(2,2-difluoro-[1,3]dioxolo[4,5-b]pyridin-6-yl)pyrrolidine-1-carboxamide

### Step 1: tert-butyl 5-(6-(1-(tert-butoxycarbonyl)pyrrolidin-2-yl)-5-fluoropyridin-3-yl)-1H-benzo[d][1,2,3]triazole-1-carboxylate

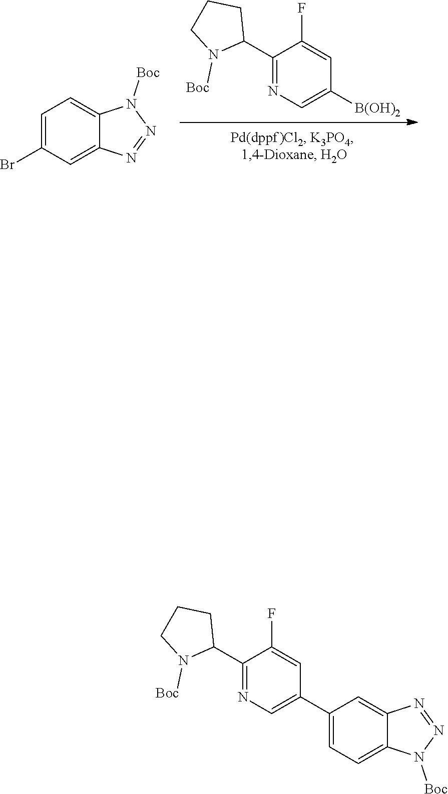 [SMILES: CC(C)(C)OC(=O)n1nnc2cc(Br)ccc21 ; CC(C)(C)OC(=O)N1CCCC1c1ncc(B(O)O)cc1F ; CC(C)(C)OC(=O)N1CCCC1c1ncc(-c2ccc3c(c2)nnn3C(=O)OC(C)(C)C)cc1F]

To tert-butyl 5-bromo-1,2,3-benzotriazole-1-carboxylate (800 mg, 2.68 mmol), and 6-[l-(tert-butoxycarbonyl)pyrrolidin-2-yl]-5-fluoropyridin-3-ylboronic acid (832 mg, 2.69 mmol) in a solution of 1,4-dioxane (6.0 mL) and water (0.5 mL) was added Pd(dppf)Cl₂ (219 mg, 0.268 mmol) and tribasic potassium phosphate (1.71 g, 8.05 mmol), and the resultant solution was stirred at 80° C. for 2 hours. Upon completion, the reaction mixture was cooled to ambient temperature and concentrated under reduced pressure. The crude residue was purified by silica gel chromatography, eluted with petroleum ether/ethyl acetate (20:1), to afford tert-butyl 5-(6-(1-(tert-butoxycarbonyl)pyrrolidin-2-yl)-5-fluoropyridin-3-yl)-1H-benzo[d][1,2,3]triazole-1-carboxylate (410 mg, 32%) as a brown oil. LCMS (ESI) [M+H]⁺: 484.5

### Step 2: 5-(5-fluoro-6-(pyrrolidin-2-yl)pyridin-3-yl)-1H-benzo[d][1,2,3]triazole

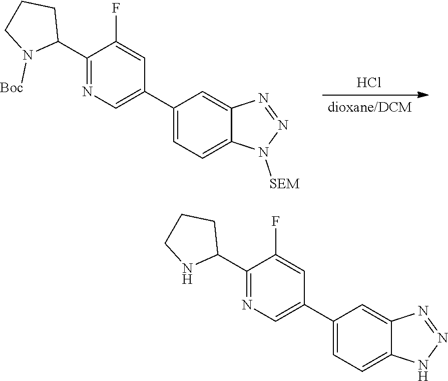

To a solution of tert-butyl 5-(6-(1-(tert-butoxycarbonyl)pyrrolidin-2-yl)-5-fluoropyridin-3-yl)-1H-benzo[d][1,2,3]triazole-1-carboxylate (400 mg, 0.827 mmol) in dichloromethane (5.0 mL) at room temperature was added a solution of HCl (1.00 mL, 2.00 mmol, 2.0 M in 1,4-dioxane), and the resultant mixture was stirred for 2 hours. Upon completion, the mixture was concentrated under reduced pressure to afford 5-(5-fluoro-6-(pyrrolidin-2-yl)pyridin-3-yl)-1H-benzo[d][1,2,3]triazole (320 mg, crude) as a brown semi-solid, which was used directly in the next step without further purification. LCMS (ESI) [M+H]⁺: 284.3

### Step 3: 2-(5-(1H-benzo[d][1,2,3]triazol-5-yl)-3-fluoropyridin-2-yl)-N-(2,2-difluoro-[1,3]dioxolo[4,5-b]pyridin-6-yl)pyrrolidine-1-carboxamide

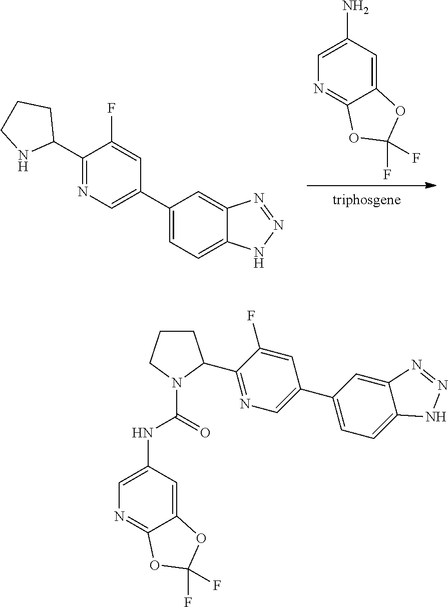 [SMILES: Fc1cc(-c2ccc3[nH]nnc3c2)cnc1C1CCCN1 ; Nc1cnc2c(c1)OC(F)(F)O2 ; O=C(Nc1cnc2c(c1)OC(F)(F)O2)N1CCCC1c1ncc(-c2ccc3[nH]nnc3c2)cc1F]

A mixture of 5-(5-fluoro-6-(pyrrolidin-2-yl)pyridin-3-yl)-1H-benzo[d][1,2,3]triazole (130 mg, 0.459 mmol), 2,2-difluoro-[1,3]dioxolo[4,5-b]pyridin-6-amine (80 mg, 0.459 mmol), N, N-diisopropylethylamine (297 mg, 2.30 mmol) and triphosgene (54.5 mg, 0.184 mmol) in dichloromethane (3.0 mL) was stirred at room temperature for 1 hour. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford 2-(5-(1H-benzo[d][1,2,3]triazol-5-yl)-3-fluoropyridin-2-yl)-N-(2,2-difluoro-[1,3]dioxolo[4,5-b]pyridin-6-yl)pyrrolidine-1-carboxamide (55 mg, 25%) as an off-white solid. LCMS (ESI) [M+H]⁺: 484.4

### Step 4: (S)-2-(5-(1H-benzo[d][1,2,3]triazol-5-yl)-3-fluoropyridin-2-yl)-N-(2,2-difluoro-[1,3]dioxolo[4,5-b]pyridin-6-yl)pyrrolidine-1-carboxamide and (R)-2-(5-(1H-benzo[d][1,2,3]triazol-5-yl)-3-fluoropyridin-2-yl)-N-(2,2-difluoro-[1,3]dioxolo[4,5-b]pyridin-6-yl)pyrrolidine-1-carboxamide

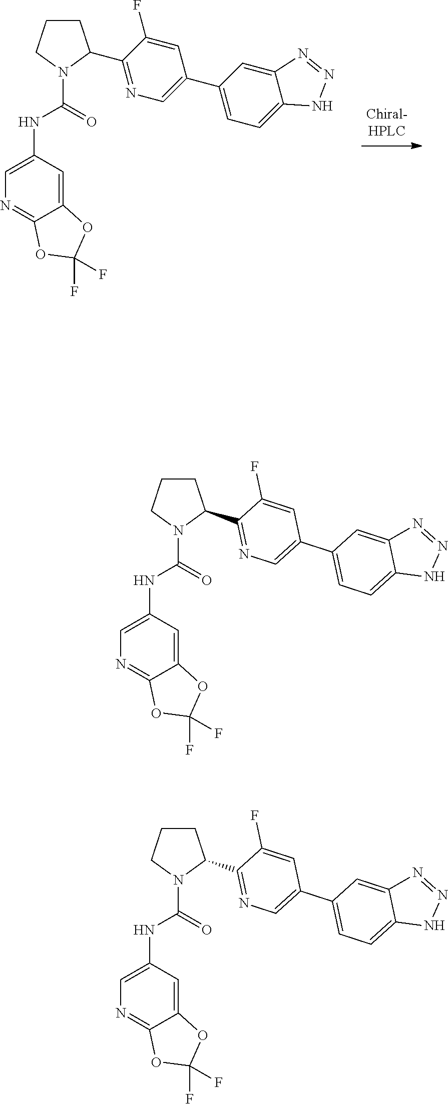 [SMILES: O=C(Nc1cnc2c(c1)OC(F)(F)O2)N1CCCC1c1ncc(-c2ccc3[nH]nnc3c2)cc1F ; O=C(Nc1cnc2c(c1)OC(F)(F)O2)N1CCC[C@H]1c1ncc(-c2ccc3[nH]nnc3c2)cc1F ; O=C(Nc1cnc2c(c1)OC(F)(F)O2)N1CCC[C@@H]1c1ncc(-c2ccc3[nH]nnc3c2)cc1F]

2-(5-(1H-benzo[d][1,2,3]triazol-5-yl)-3-fluoropyridin-2-yl)-N-(2,2-difluoro-[1,3]dioxolo[4,5-b]pyridin-6-yl)pyrrolidine-1-carboxamide (50.0 mg) was resolved by prep chiral-HPLC (conditions: column: CHIRAL ART Cellulose-SC, 3*25 cm, 5 μm; mobile phase A: hexane (0.1% formic acid); mobile phase B: ethanol; Flow rate: 40 mL/min; gradient (B %): isocratic 30% B; detector: 220/254 nm; RT1 (min): 11.2; RT2 (min): 17.2) to afford (Compound 164a; Method AS, Peak 1, 0.81 min) (S)-2-(5-(1H-benzo[d][1,2,3]triazol-5-yl)-3-fluoropyridin-2-yl)-N-(2,2-difluoro-[1,3]dioxolo[4,5-b]pyridin-6-yl)pyrrolidine-1-carboxamide (1^{st} peak, 13.8 mg, 28%) as a white solid and (Compound 164b; Method AS, Peak 2, 2.17 min) (R)-2-(5-(1H-benzo[d][1,2,3]triazol-5-yl)-3-fluoropyridin-2-yl)-N-(2,2-difluoro-[1,3]dioxolo[4,5-b]pyridin-6-yl)pyrrolidine-1-carboxamide (2^{nd} peak, 17.0 mg, 34%) as a white solid.

| Cmpd # | Characterization Data |
|---|---|
| 164a | LCMS (ESI) [M + H]⁺: 484.2 |
| | ¹H NMR (400 MHz, DMSO-d₆) δ 8.79 (s, 1H), 8.73 (s, 1H), 8.31 (s, 1H), 8.13 (dd, J = 11.6, 1.9 |
| | Hz, 1H), 8.07-7.96 (m, 3H), 7.83 (dd, J = 8.7, 1.6 Hz, 1H), 5.43-5.28 (m, 1H), 3.81-3.74 |
| | (m, 1H), 3.71-3.61 (m, 1H), 2.40-2.32 (m, 1H), 2.18-2.11 (m, 1H), 2.08-1.98 (m, 1H), |
| | 1.98-1.87 (m, 1H). |
| 164b | LCMS (ESI) [M + H]⁺: 484.2 |
| | ¹H NMR (400 MHz, DMSO-d₆) δ 8.79 (s, 1H), 8.73 (s, 1H), 8.31 (s, 1H), 8.12 (dd, J = 11.5, 1.9 |
| | Hz, 1H), 8.07-7.95 (m, 3H), 7.82 (d, J = 8.7 Hz, 1H), 5.40-5.32 (m, 1H), 3.81-3.75 (m, |
| | 1H), 3.70-3.62 (m, 1H), 2.42-2.33 (m, 1H), 2.17-2.10 (m, 1H), 2.08-2.01 (m, 1H), 1.97- |
| | 1.90 (m, 1H). |

### Example 165: (S)-2-(5-(1H-benzo[d][1,2,3]triazol-5-yl)-3-fluoropyridin-2-yl)-N-(2,2-difluoro-[1,3]dioxolo[4,5-b]pyridin-5-yl)pyrrolidine-1-carboxamide and (R)-2-(5-(1H-benzo[d][1,2,3]triazol-5-yl)-3-fluoropyridin-2-yl)-N-(2,2-difluoro-[1,3]dioxolo[4,5-b]pyridin-5-yl)pyrrolidine-1-carboxamide

### Step 1: 2-(5-(1H-benzo[d][1,2,3]triazol-5-yl)-3-fluoropyridin-2-yl)-N-(2,2-difluoro-[1,3]dioxolo[4,5-b]pyridin-5-yl)pyrrolidine-1-carboxamide

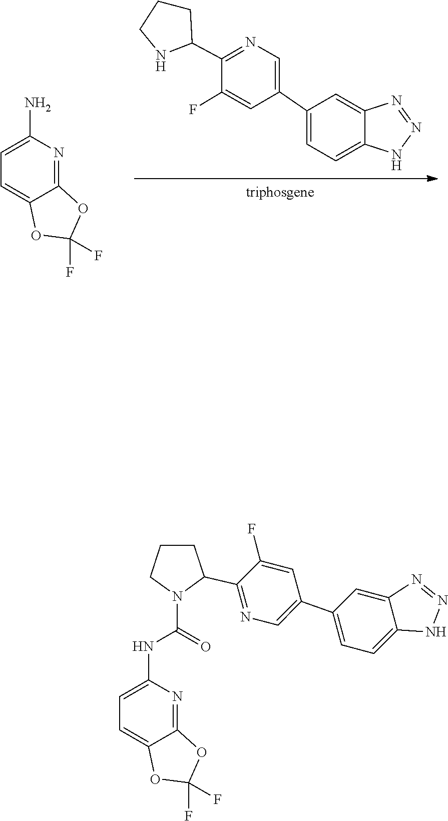 [SMILES: Nc1ccc2c(n1)OC(F)(F)O2 ; Fc1cc(-c2ccc3[nH]nnc3c2)cnc1C1CCCN1 ; O=C(Nc1ccc2c(n1)OC(F)(F)O2)N1CCCC1c1ncc(-c2ccc3[nH]nnc3c2)cc1F]

A solution of 5-[5-fluoro-6-(pyrrolidin-2-yl)pyridin-3-yl]-1H-1,2,3-benzotriazole (130 mg, 0.459 mmol), 2,2-difluoro-[1,3]dioxolo[4,5-b]pyridin-5-amine (80.0 mg, 0.459 mmol), N,N-diisopropylethylamine (297 mg, 2.30 mmol) and triphosgene (54.5 mg, 0.184 mmol) in dichloromethane (3.0 mL) was stirred at room temperature for 1 hour. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to give 2-(5-(1H-benzo[d][1,2,3]triazol-5-yl)-3-fluoropyridin-2-yl)-N-(2,2-difluoro-[1,3]dioxolo[4,5-b]pyridin-5-yl)pyrrolidine-1-carboxamide (65.0 mg, 29%) as an off-white solid. LCMS (ESI) [M+H]⁺: 484.4

### Step 2: (S)-2-(5-(1H-benzo[d][1,2,3]triazol-5-yl)-3-fluoropyridin-2-yl)-N-(2,2-difluoro-[1,3]dioxolo[4,5-b]pyridin-5-yl)pyrrolidine-1-carboxamide and (R)-2-(5-(1H-benzo[d][1,2,3]triazol-5-yl)-3-fluoropyridin-2-yl)-N-(2,2-difluoro-[1,3]dioxolo[4,5-b]pyridin-5-yl)pyrrolidine-1-carboxamide

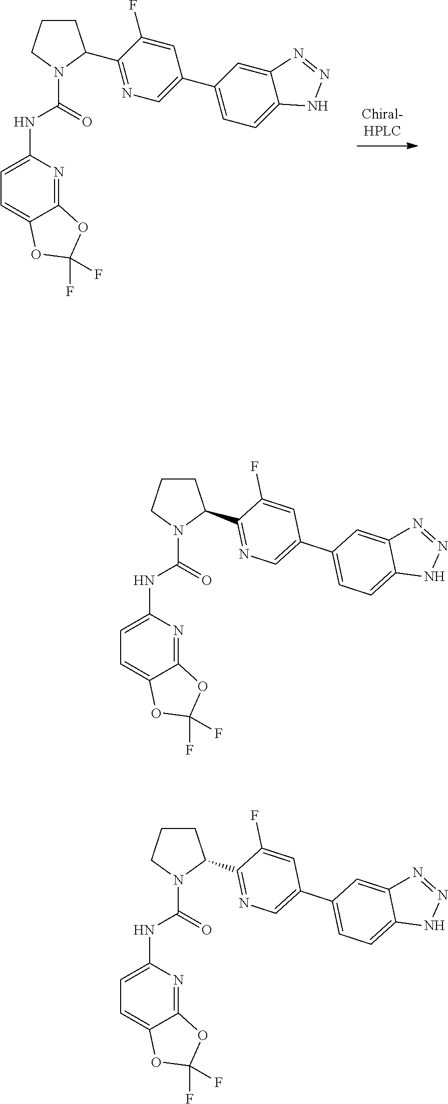 [SMILES: O=C(Nc1ccc2c(n1)OC(F)(F)O2)N1CCCC1c1ncc(-c2ccc3[nH]nnc3c2)cc1F ; O=C(Nc1ccc2c(n1)OC(F)(F)O2)N1CCC[C@H]1c1ncc(-c2ccc3[nH]nnc3c2)cc1F ; O=C(Nc1ccc2c(n1)OC(F)(F)O2)N1CCC[C@@H]1c1ncc(-c2ccc3[nH]nnc3c2)cc1F]

2-(5-(1H-benzo[d][1,2,3]triazol-5-yl)-3-fluoropyridin-2-yl)-N-(2,2-difluoro-[1,3]dioxolo[4,5-b]pyridin-5-yl)pyrrolidine-1-carboxamide (60 mg) was resolved by chiral-HPLC (conditions: column: CHIRALPAK IH3*25 cm, 5 μm; mobile phase A: hexane (0.5% 2M NH₃/methanol); mobile phase B: ethanol; Flow rate: 40 mL/min; gradient (B %): isocratic 40% B; detector: 220/254 nm; RT1 (min): 8.8; RT2 (min): 15.1) to afford (Compound 165a; Method AT, Peak 1, 1.28 min) (S)-2-(5-(1H-benzo[d][1,2,3]triazol-5-yl)-3-fluoropyridin-2-yl)-N-(2,2-difluoro-[1,3]dioxolo[4,5-b]pyridin-5-yl)pyrrolidine-1-carboxamide (1^{st} peak, 24.5 mg, 41%) as a white solid and (Compound 165b; Method AT, Peak 2, 2.28 min) (R)-2-(5-(1H-benzo[d][1,2,3]triazol-5-yl)-3-fluoropyridin-2-yl)-N-(2,2-difluoro-[1,3]dioxolo[4,5-b]pyridin-5-yl)pyrrolidine-1-carboxamide (26.8 mg, 45%) as a white solid.

| Cmpd # | Characterization Data |
|---|---|
| 165a | LCMS (ESI) [M + H]⁺: 484.2 |
| | ¹H NMR (400 MHz, DMSO-d₆) δ 9.10 (s, 1H), 8.79 (s, 1H), 8.31 (s, 1H), 8.13 (d, J = 11.6 Hz, |
| | 1H), 8.01 (d, J = 8.7 Hz, 1H), 7.83 (d, J = 8.7 Hz, 1H), 7.73 (d, J = 8.8 Hz, 1H), 7.65 (d, J = 8.8 |
| | Hz, 1H), 5.46-5.34 (m, 1H), 3.85-3.77 (m, 1H), 3.71-3.62 (m, 1H), 2.39-2.30 (m, 1H), |
| | 2.12-1.89 (m, 3H). |
| 165b | LCMS (ESI) [M + H]⁺: 484.2 |
| | ¹H NMR (400 MHz, DMSO-d₆) δ 9.10 (s, 1H), 8.78 (s, 1H), 8.30 (s, 1H), 8.13 (d, J = 11.7 Hz, |
| | 1H), 8.00 (d, J = 8.6 Hz, 1H), 7.82 (d, J = 8.7 Hz, 1H), 7.73 (d, J = 8.8 Hz, 1H), 7.65 (d, J = 8.8 |
| | Hz, 1H), 5.50-5.26 (m, 1H), 3.86-3.77 (m, 1H), 3.72-3.64 (m, 1H), 2.40-2.30 (m, 1H), |
| | 2.11-1.88 (m, 3H). |

### Example 166: 4-(5-fluoro-6-((2S,4S)-1-((5-fluoro-6-(1-(trifluoromethyl)cyclopropyl)pyridin-3-yl)carbamoyl)-4-methylpyrrolidin-2-yl)pyridin-3-yl)benzoic acid and 4-(5-fluoro-6-((2R,4S)-1-((5-fluoro-6-(1-(trifluoromethyl)cyclopropyl)pyridin-3-yl)carbamoyl)-4-methylpyrrolidin-2-yl)pyridin-3-yl)benzoic acid

### Step 1: tert-butyl (4S)-2-(3-fluoro-5-(4-(methoxycarbonyl)phenyl)pyridin-2-yl)-4-methylpyrrolidine-1-carboxylate

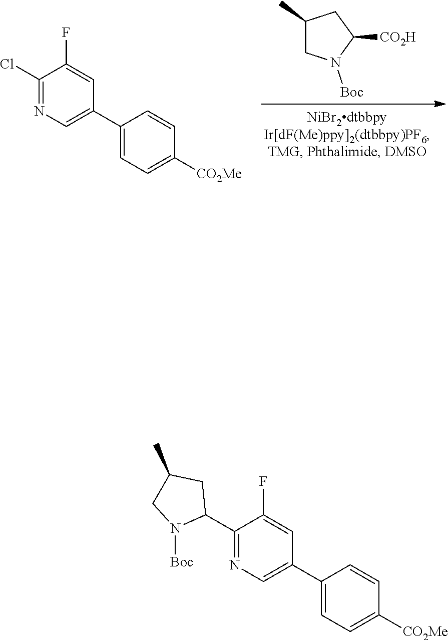 [SMILES: COC(=O)c1ccc(-c2cnc(Cl)c(F)c2)cc1 ; C[C@H]1C[C@@H](C(=O)O)N(C(=O)OC(C)(C)C)C1 ; COC(=O)c1ccc(-c2cnc(C3C[C@H](C)CN3C(=O)OC(C)(C)C)c(F)c2)cc1]

A solution of methyl 4-(6-chloro-5-fluoropyridin-3-yl)benzoate (300 mg, 1.13 mmol), (2S,4S)-1-(tert-butoxycarbonyl)-4-methylpyrrolidine-2-carboxylic acid (259 mg, 1.13 mmol), phthalimide (166 mg, 1.13 mmol), [4,4′-Bis(tert-butyl)-2,2′-bipyridine]nickel dibromide (55 mg, 0.113 mmol), 1,1,3,3-tetramethylguanidine (156 mg, 1.36 mmol), and [Ir(dF(Me)ppy)₂(dtbbpy)]PF₆ (22.9 mg, 0.0230 mmol) in DMSO (6.0 mL) at room temperature was stirred under 450 nm blue LED irradiation for 3 hours. Upon completion, the mixture was diluted with water (10 mL) and extracted with ethyl acetate (3×10 mL). The combined organic layers were concentrated under reduced pressure. The crude residue was purified by silica gel column chromatography, eluted with petroleum ether/ethyl acetate (1:1), to afford tert-butyl (4S)-2-(3-fluoro-5-(4-(methoxycarbonyl)phenyl)pyridin-2-yl)-4-methylpyrrolidine-1-carboxylate (300 mg, 64%) as a yellow oil. LCMS (ESI) [M+H]⁺: 415.2

### Step 2: methyl 4-(5-fluoro-6-((4S)-4-methylpyrrolidin-2-yl)pyridin-3-yl)benzoate

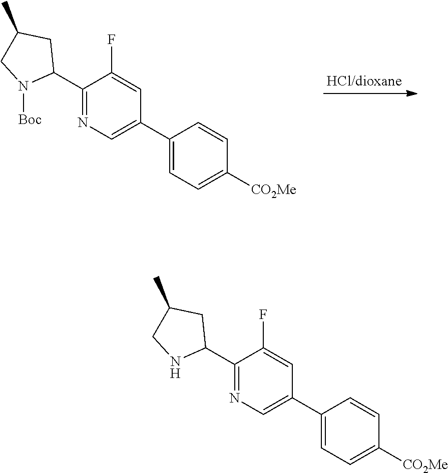 [SMILES: COC(=O)c1ccc(-c2cnc(C3C[C@H](C)CN3C(=O)OC(C)(C)C)c(F)c2)cc1 ; COC(=O)c1ccc(-c2cnc(C3C[C@H](C)CN3)c(F)c2)cc1]

To a solution of tert-butyl (4S)-2-(3-fluoro-5-(4-(methoxycarbonyl)phenyl)pyridin-2-yl)-4-methylpyrrolidine-1-carboxylate (300 mg, 0.724 mmol) in 1,4-dioxane (1.0 mL) was added a solution of HCl (3.0 mL, 6.0 mmol, 2.0 M in 1,4-dioxane), and the resultant mixture was stirred at room temperature for 30 minutes. Upon completion, the mixture was concentrated under reduced pressure to afford methyl 4-(5-fluoro-6-((4S)-4-methylpyrrolidin-2-yl)pyridin-3-yl)benzoate (238 mg, crude) as a yellow oil, which was used directly in the next step without further purification. LCMS (ESI) [M+H]⁺: 315.1

### Step 3: methyl 4-(5-fluoro-6-((4S)-1-((5-fluoro-6-(1-(trifluoromethyl)cyclopropyl)pyridin-3-yl)carbamoyl)-4-methylpyrrolidin-2-yl)pyridin-3-yl)benzoate

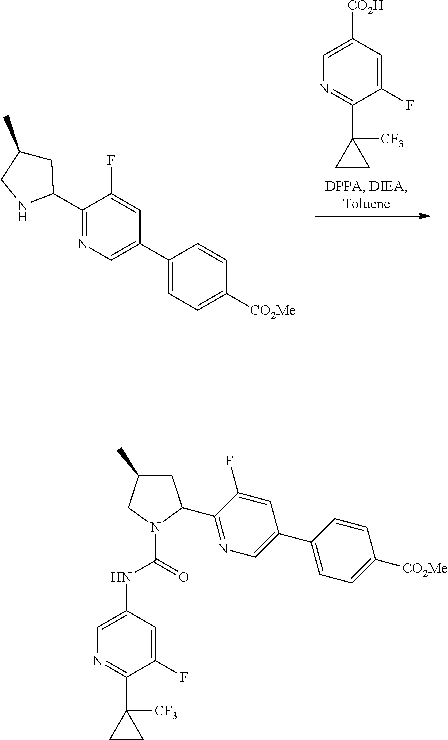 [SMILES: COC(=O)c1ccc(-c2cnc(C3C[C@H](C)CN3)c(F)c2)cc1 ; O=C(O)c1cnc(C2(C(F)(F)F)CC2)c(F)c1 ; COC(=O)c1ccc(-c2cnc(C3C[C@H](C)CN3C(=O)Nc3cnc(C4(C(F)(F)F)CC4)c(F)c3)c(F)c2)cc1]

A solution of methyl 4-(5-fluoro-6-((4S)-4-methylpyrrolidin-2-yl)pyridin-3-yl)benzoate (238 mg, 0.757 mmol), 5-fluoro-6-[1-(trifluoromethyl)cyclopropyl]pyridine-3-carboxylic acid (189 mg, 0.757 mmol), DPPA (212 mL, 0.770 mmol) and triethylamine (78.0 mg, 0.770 mmol) in toluene (3.0 mL) was stirred at 85° C. for 1 hour. Upon completion, the mixture was concentrated under reduced pressure. The crude residue was purified by silica gel column chromatography, eluted with petroleum ether/ethyl acetate (1:1) to afford methyl 4-(5-fluoro-6-((4S)-1-((5-fluoro-6-(1-(trifluoromethyl)cyclopropyl)pyridin-3-yl)carbamoyl)-4-methylpyrrolidin-2-yl)pyridin-3-yl)benzoate (90 mg, 21% yield) as a yellow oil. LCMS (ESI) [M+H]⁺: 561.1

### Step 4: 4-(5-fluoro-6-((4S)-1-((5-fluoro-6-(1-(trifluoromethyl)cyclopropyl)pyridin-3-yl)carbamoyl)-4-methylpyrrolidin-2-yl)pyridin-3-yl)benzoic acid

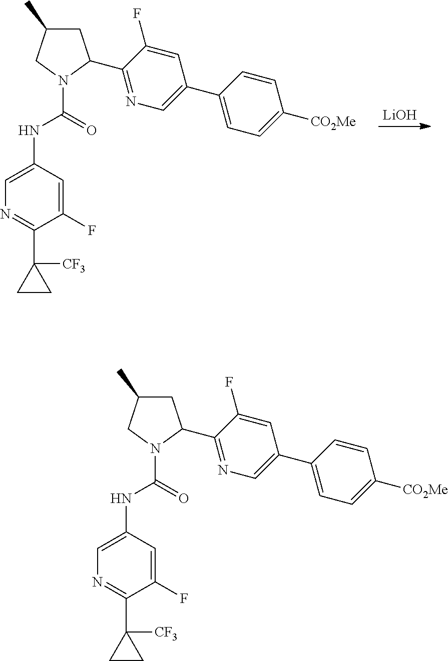 [SMILES: COC(=O)c1ccc(-c2cnc(C3C[C@H](C)CN3C(=O)Nc3cnc(C4(C(F)(F)F)CC4)c(F)c3)c(F)c2)cc1 ; "LiOH" ; COC(=O)c1ccc(-c2cnc(C3C[C@H](C)CN3C(=O)Nc3cnc(C4(C(F)(F)F)CC4)c(F)c3)c(F)c2)cc1]

A solution of methyl 4-(5-fluoro-6-((4S)-1-((5-fluoro-6-(1-(trifluoromethyl)cyclopropyl)pyridin-3-yl)carbamoyl)-4-methylpyrrolidin-2-yl)pyridin-3-yl)benzoate (60 mg, 0.107 mmol) and lithium hydroxide monohydrate (8.98 mg, 0.214 mmol) in methanol (2.5 mL), THF (2.5 mL) and water (6.0 mL) was stirred at room temperature for 1 hour. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford 4-(5-fluoro-6-((4S)-1-((5-fluoro-6-(1-(trifluoromethyl)cyclopropyl)pyridin-3-yl)carbamoyl)-4-methylpyrrolidin-2-yl)pyridin-3-yl)benzoic acid (40.0 mg, 68%) as a white solid. LCMS (ESI) [M+H]⁺: 547.1

### Step 5: 4-(5-fluoro-6-((2S,4S)-1-((5-fluoro-6-(1-(trifluoromethyl)cyclopropyl)pyridin-3-yl)carbamoyl)-4-methylpyrrolidin-2-yl)pyridin-3-yl)benzoic acid and 4-(5-fluoro-6-((2R,4S)-1-((5-fluoro-6-(1-(trifluoromethyl)cyclopropyl)pyridin-3-yl)carbamoyl)-4-methylpyrrolidin-2-yl)pyridin-3-yl)benzoic acid

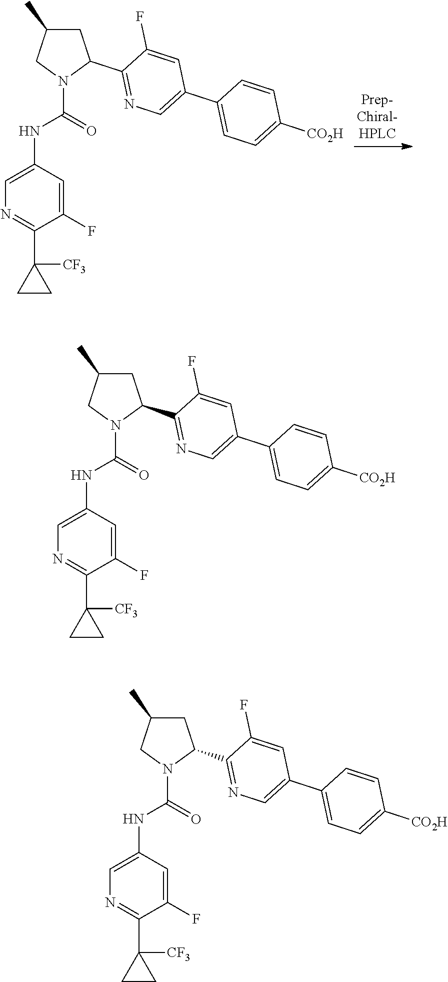 [SMILES: C[C@H]1CC(c2ncc(-c3ccc(C(=O)O)cc3)cc2F)N(C(=O)Nc2cnc(C3(C(F)(F)F)CC3)c(F)c2)C1 ; C[C@H]1C[C@@H](c2ncc(-c3ccc(C(=O)O)cc3)cc2F)N(C(=O)Nc2cnc(C3(C(F)(F)F)CC3)c(F)c2)C1 ; C[C@H]1C[C@H](c2ncc(-c3ccc(C(=O)O)cc3)cc2F)N(C(=O)Nc2cnc(C3(C(F)(F)F)CC3)c(F)c2)C1]

4-(5-Fluoro-6-((4S)-1-((5-fluoro-6-(1-(trifluoromethyl)cyclopropyl)pyridin-3-yl)carbamoyl)-4-methylpyrrolidin-2-yl)pyridin-3-yl)benzoic acid (40 mg) was resolved by chiral HPLC (conditions: column: CHIRAL ART Cellulose-SC, 3*25 cm, 5 μm; mobile phase A: hexane (0.1% formic acid); mobile phase B: ethanol; flow rate: 40 mL/min; gradient (B %): isocratic 30% B; detector: 220/254 nm; RT1 (min): 6.5; RT2 (min): 9.5) to afford (Compound 166a; Method A, Peak 1, 0.70 min) 4-(5-fluoro-6-((2S,4S)-1-((5-fluoro-6-(1-(trifluoromethyl)cyclopropyl)pyridin-3-yl)carbamoyl)-4-methylpyrrolidin-2-yl)pyridin-3-yl)benzoic acid (1^{st} peak, 4.0 mg, 6.8%) as a white solid and (Compound 166b; Method A, Peak 2, 1.27 min) 4-(5-fluoro-6-((2R,4S)-1-((5-fluoro-6-(1-(trifluoromethyl)cyclopropyl)pyridin-3-yl)carbamoyl)-4-methylpyrrolidin-2-yl)pyridin-3-yl)benzoic acid (2^{nd} peak, 23.0 mg, 58%) as a white solid.

| Cmpd # | Characterization Data |
|---|---|
| 166a | LCMS (ESI) [M + H]⁺: 547.2 |
| | ¹H NMR (400 MHz, methanol-d₄) δ 8.64 (s, 1H), 8.38 (d, J = 2.2 Hz, 1H), 8.12 (d, J = 8.2 Hz, |
| | 2H), 7.89 (dd, J = 11.2, 1.9 Hz, 1H), 7.83 (dd, J = 12.2, 2.2 Hz, 1H), 7.77 (d, J = 8.3 Hz, 2H), |
| | 5.40 (dd, J = 9.7, 6.9 Hz, 1H), 3.95 (t, J = 8.4 Hz, 1H), 3.38 (t, J = 9.8 Hz, 1H), 2.66-2.43 |
| | (m, 2H), 1.71 (q, J = 11.0 Hz, 1H), 1.46-1.34 (m, 2H), 1.17 (dd, J = 14.6, 5.7 Hz, 5H). |
| 166b | LCMS (ESI) [M + H]⁺: 547.2 |
| | ¹H NMR (400 MHz, Methanol-d₄) δ 8.61 (s, 1H), 8.42 (s, 1H), 8.15-8.08 (m, 2H), 7.88 (dt, |
| | J = 11.7, 2.3 Hz, 2H), 7.74 (dd, J = 8.4, 2.8 Hz, 2H), 5.52 (t, J = 5.4 Hz, 1H), 4.03 (dd, J = |
| | 9.3, 7.5 Hz, 1H), 3.29-3.21 (m, 1H), 2.64 (d, J = 13.7 Hz, 1H), 2.11 (s, 2H), 1.40 (s, 2H), |
| | 1.20-1.09 (m, 5H). |

### Example 167: 4-(5-fluoro-6-((2R,4R)-1-((5-fluoro-6-(1-(trifluoromethyl)cyclopropyl)pyridin-3-yl)carbamoyl)-4-methylpyrrolidin-2-yl)pyridin-3-yl)benzoic acid and 4-(5-fluoro-6-((2S,4R)-1-((5-fluoro-6-(1-(trifluoromethyl)cyclopropyl)pyridin-3-yl)carbamoyl)-4-methylpyrrolidin-2-yl)pyridin-3-yl)benzoic acid

### Step 1: tert-butyl (4R)-2-(3-fluoro-5-(4-(methoxycarbonyl)phenyl)pyridin-2-yl)-4-methylpyrrolidine-1-carboxylate

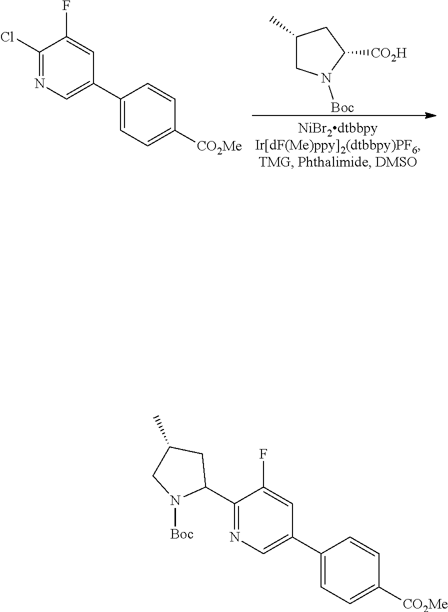 [SMILES: COC(=O)c1ccc(-c2cnc(Cl)c(F)c2)cc1 ; C[C@@H]1C[C@H](C(=O)O)N(C(=O)OC(C)(C)C)C1 ; COC(=O)c1ccc(-c2cnc(C3C[C@@H](C)CN3C(=O)OC(C)(C)C)c(F)c2)cc1]

A solution of methyl 4-(6-chloro-5-fluoropyridin-3-yl)benzoate (300 mg, 1.13 mmol), (2R,4R)-1-(tert-butoxycarbonyl)-4-methylpyrrolidine-2-carboxylic acid (259 mg, 1.13 mmol), phthalimide (166 mg, 1.13 mmol), [4,4′-Bis(tert-butyl)-2,2′-bipyridine]nickel dibromide (55 mg, 0.113 mmol), 1,1,3,3-tetramethylguanidine (156 mg, 1.36 mmol) and [Ir(dF(Me)ppy)₂(dtbbpy)]PF₆ (22.9 mg, 0.023 mmol) in DMSO (6.0 mL) at room temperature was stirred for 3 hours under 450 nm blue LED irradiation. Upon completion, the mixture was diluted with water (10 mL) and extracted with ethyl acetate (3×10 mL). The combined organic layers were concentrated under reduced pressure. The crude residue was purified by silica gel column chromatography, eluted with petroleum ether/ethyl acetate (1:1) to afford tert-butyl (4R)-2-(3-fluoro-5-(4-(methoxycarbonyl)phenyl)pyridin-2-yl)-4-methylpyrrolidine-1-carboxylate (258 mg, 55%) as a yellow oil. LCMS (ESI) [M+H]⁺: 415.2.

### Step 2: Methyl 4-(5-fluoro-6-((4R)-4-methylpyrrolidin-2-yl)pyridin-3-yl)benzoate

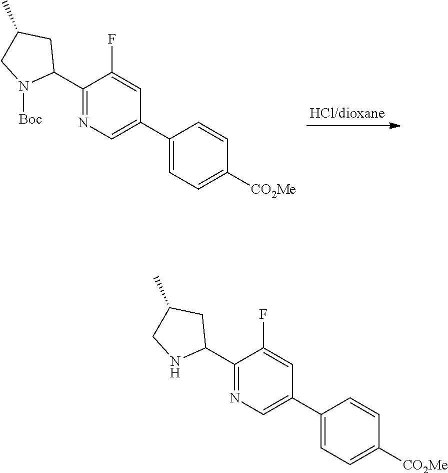 [SMILES: COC(=O)c1ccc(-c2cnc(C3C[C@@H](C)CN3C(=O)OC(C)(C)C)c(F)c2)cc1 ; COC(=O)c1ccc(-c2cnc(C3C[C@@H](C)CN3)c(F)c2)cc1]

To a solution of tert-butyl (4R)-2-(3-fluoro-5-(4-(methoxycarbonyl)phenyl)pyridin-2-yl)-4-methylpyrrolidine-1-carboxylate (258 mg, 0.622 mmol) in 1,4-dioxane (1.0 mL) was added a solution of HCl (2.0 mL, 4.0 mmol, 2.0 M in 1,4-dioxane) and the resultant mixture was stirred at room temperature for 30 minutes. Upon completion, the mixture was concentrated under reduced pressure to afford methyl 4-(5-fluoro-6-((4R)-4-methylpyrrolidin-2-yl)pyridin-3-yl)benzoate (200 mg, crude) as a yellow oil, which was used directly in the next step without further purification. LCMS (ESI) [M+H]⁺: 315.1

### Step 3: methyl 4-(5-fluoro-6-((4R)-1-((5-fluoro-6-(1-(trifluoromethyl)cyclopropyl)pyridin-3-yl)carbamoyl)-4-methylpyrrolidin-2-yl)pyridin-3-yl)benzoate

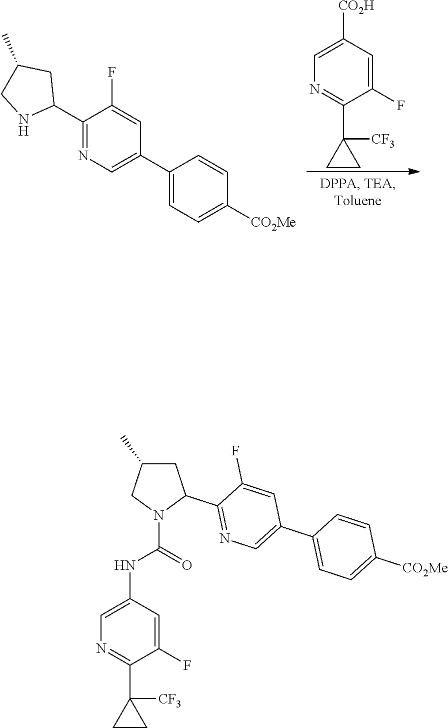 [SMILES: COC(=O)c1ccc(-c2cnc(C3C[C@@H](C)CN3)c(F)c2)cc1 ; O=C(O)c1cnc(C2(C(F)(F)F)CC2)c(F)c1 ; COC(=O)c1ccc(-c2cnc(C3C[C@@H](C)CN3C(=O)Nc3cnc(C4(C(F)(F)F)CC4)c(F)c3)c(F)c2)cc1]

A solution of methyl 4-(5-fluoro-6-((4R)-4-methylpyrrolidin-2-yl)pyridin-3-yl)benzoate (200 mg, 0.636 mmol), 5-fluoro-6-[1-(trifluoromethyl)cyclopropyl]pyridine-3-carboxylic acid (106 mg, 0.424 mmol), DPPA (212 mL, 0.770 mmol) and triethylamine (78 mg, 0.770 mmol) in toluene (3.0 mL) was stirred at 85° C. for 1 hour. Upon completion, the mixture was concentrated under reduced pressure. The crude residue was purified by silica gel column chromatography, eluted with petroleum ether/ethyl acetate (1:1) to afford methyl 4-(5-fluoro-6-((4R)-1-((5-fluoro-6-(1-(trifluoromethyl)cyclopropyl)pyridin-3-yl)carbamoyl)-4-methylpyrrolidin-2-yl)pyridin-3-yl)benzoate (70 mg, 29%) as a yellow oil. LCMS (ESI) [M+H]⁺: 561.1

### Step 4: 4-(5-fluoro-6-((4R)-1-((5-fluoro-6-(1-(trifluoromethyl)cyclopropyl)pyridin-3-yl)carbamoyl)-4-methylpyrrolidin-2-yl)pyridin-3-yl)benzoic acid

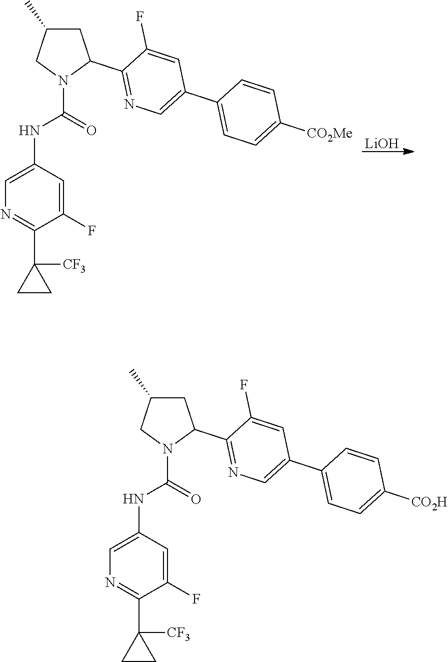 [SMILES: COC(=O)c1ccc(-c2cnc(C3C[C@@H](C)CN3C(=O)Nc3cnc(C4(C(F)(F)F)CC4)c(F)c3)c(F)c2)cc1 ; "LiOH" ; C[C@@H]1CC(c2ncc(-c3ccc(C(=O)O)cc3)cc2F)N(C(=O)Nc2cnc(C3(C(F)(F)F)CC3)c(F)c2)C1]

A solution of methyl 4-(5-fluoro-6-((4R)-1-((5-fluoro-6-(1-(trifluoromethyl)cyclopropyl)pyridin-3-yl)carbamoyl)-4-methylpyrrolidin-2-yl)pyridin-3-yl)benzoate (70.0 mg, 0.125 mmol) and lithium hydroxide monohydrate (26.2 mg, 0.625 mmol) in methanol (2.5 mL), THF (2.5 mL) and water (6.0 mL) was stirred at room temperature for 1 hour. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford 4-(5-fluoro-6-((4R)-1-((5-fluoro-6-(1-(trifluoromethyl)cyclopropyl)pyridin-3-yl)carbamoyl)-4-methylpyrrolidin-2-yl)pyridin-3-yl)benzoic acid (40.0 mg, 59%) as a white solid. LCMS (ESI) [M+H]⁺: 547.1

### Step 5: 4-(5-fluoro-6-((2R,4R)-1-((5-fluoro-6-(1-(trifluoromethyl)cyclopropyl)pyridin-3-yl)carbamoyl)-4-methylpyrrolidin-2-yl)pyridin-3-yl)benzoic acid and 4-(5-fluoro-6-((2S,4R)-1-((5-fluoro-6-(1-(trifluoromethyl)cyclopropyl)pyridin-3-yl)carbamoyl)-4-methylpyrrolidin-2-yl)pyridin-3-yl)benzoic acid

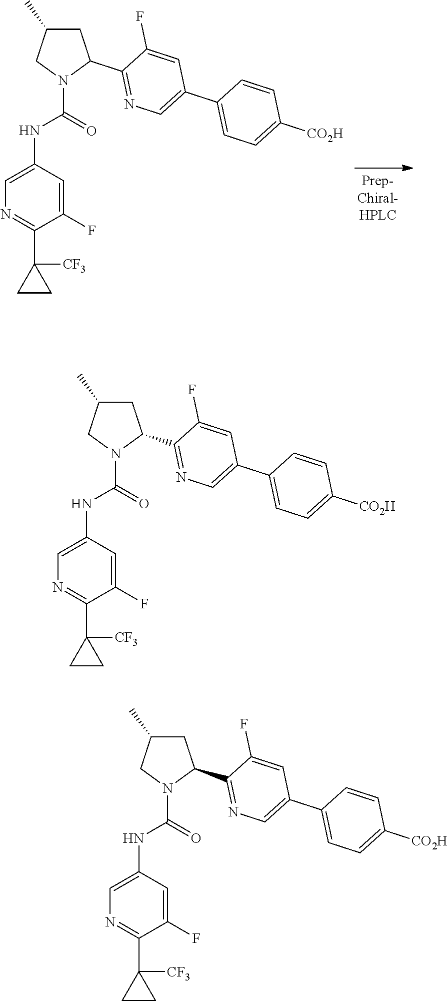 [SMILES: C[C@@H]1CC(c2ncc(-c3ccc(C(=O)O)cc3)cc2F)N(C(=O)Nc2cnc(C3(C(F)(F)F)CC3)c(F)c2)C1 ; C[C@@H]1C[C@H](c2ncc(-c3ccc(C(=O)O)cc3)cc2F)N(C(=O)Nc2cnc(C3(C(F)(F)F)CC3)c(F)c2)C1 ; C[C@@H]1C[C@@H](c2ncc(-c3ccc(C(=O)O)cc3)cc2F)N(C(=O)Nc2cnc(C3(C(F)(F)F)CC3)c(F)c2)C1]

4-(5-fluoro-6-((4R)-1-((5-fluoro-6-(1-(trifluoromethyl)cyclopropyl)pyridin-3-yl)carbamoyl)-4-methylpyrrolidin-2-yl)pyridin-3-yl)benzoic acid (40 mg) was resolved by prep chiral HPLC (conditions: column: CHIRAL ART Cellulose-SC, 3*25 cm, 5 μm; mobile phase A: hexane (0.1% formic acid); mobile phase B: ethanol; flow rate: 40 mL/min; gradient (B %): isocratic 25% B; detector: 220/254 nm; RT1 (min): 7.3; RT2 (min): 9.3) to afford (Compound 167a; Method A, Peak 1, 0.69 min) 4-(5-fluoro-6-((2R,4R)-1-((5-fluoro-6-(1-(trifluoromethyl)cyclopropyl)pyridin-3-yl)carbamoyl)-4-methylpyrrolidin-2-yl)pyridin-3-yl)benzoic acid (1^{st} peak, 8.3 mg, 21%) as a white solid and (Compound 167b; Method A, Peak 2, 0.97 min) 4-(5-fluoro-6-((2S,4R)-1-((5-fluoro-6-(1-(trifluoromethyl)cyclopropyl)pyridin-3-yl)carbamoyl)-4-methylpyrrolidin-2-yl)pyridin-3-yl)benzoic acid (2^{nd} peak, 24.0 mg, 60%) as a white solid.

| Cmpd # | Characterization Data |
|---|---|
| 167a | LCMS (ESI) [M + H]⁺: 547.3 |
| | ¹H NMR (300 MHz, DMSO-d₆) δ 8.84 (s, 1H), 8.73 (t, J = 1.6 Hz, 1H), 8.50 (d, J = 1.9 Hz, |
| | 1H), 8.09 (dd, J = 11.5, 1.9 Hz, 1H), 8.03 (d, J = 8.4 Hz, 2H), 7.96-7.85 (m, 3H), 5.41 (dd, |
| | J = 8.0, 3.3 Hz, 1H), 4.03-3.88 (m, 1H), 3.22 (t, J = 8.8 Hz, 2H), 2.13-1.96 (m, 2H), 1.37 (d, |
| | J = 2.3 Hz, 2H), 1.18-1.02 (m, 5H). |
| 167b | LCMS (ESI) [M + H]⁺: 547.2 |
| | ¹H NMR (300 MHz, DMSO-d₆) δ 8.91 (s, 1H), 8.74 (t, J = 1.6 Hz, 1H), 8.45 (d, J = 1.7 Hz, |
| | 1H), 8.12-7.98 (m, 3H), 7.95-7.79 (m, 3H), 5.33-5.23 (m, 1H), 3.97-3.89 (m, 1H), 3.26- |
| | 3.21 (m, 1H), 1.69-1.53 (m, 2H), 1.40-1.31 (m, 2H), 1.19-1.04 (m, 5H). |

### Example 168: 4-(6-((2R,4R)-1-((2,2-difluorobenzo[d][1,3]dioxol-5-yl)carbamoyl)-4-methylpyrrolidin-2-yl)-5-fluoropyridin-3-yl)benzoic acid and 4-(6-((2R,4S)-1-((2,2-difluorobenzo[d][1,3]dioxol-5-yl)carbamoyl)-4-methylpyrrolidin-2-yl)-5-fluoropyridin-3-yl)benzoic acid

### Step 1: methyl 4-(5-fluoro-6-((2R)-4-methylpyrrolidin-2-yl)pyridin-3-yl)benzoate

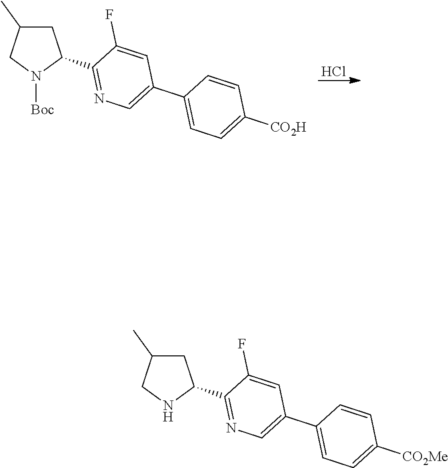 [SMILES: CC1C[C@H](c2ncc(-c3ccc(C(=O)O)cc3)cc2F)N(C(=O)OC(C)(C)C)C1 ; Cl ; COC(=O)c1ccc(-c2cnc([C@H]3CC(C)CN3)c(F)c2)cc1]

To a solution of tert-butyl (2R)-2-(3-fluoro-5-(4-(methoxycarbonyl)phenyl)pyridin-2-yl)-4-methylpyrrolidine-1-carboxylate (800 mg, 1.93 mmol) in dichloromethane (5.0 mL) at room temperature was added a solution of HCl (2.0 mL, 4. mmol, 2.0 M in 1,4-dioxane). The reaction mixture was stirred for 2 hours. Upon completion, the mixture was concentrated under reduced pressure to afford methyl 4-(5-fluoro-6-((2R)-4-methylpyrrolidin-2-yl)pyridin-3-yl)benzoate (780 mg, crude) as a brown oil, which was used directly in the next step without further purification. LCMS (ESI) [M+H]⁺: 315.3

### Step 2: 4-(5-fluoro-6-((2R)-4-methylpyrrolidin-2-yl)pyridin-3-yl)benzoic acid

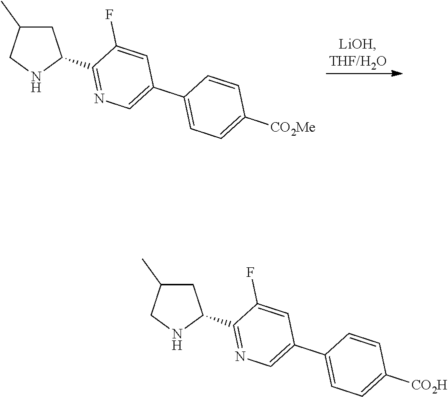 [SMILES: COC(=O)c1ccc(-c2cnc([C@H]3CC(C)CN3)c(F)c2)cc1 ; CC1CN[C@@H](c2ncc(-c3ccc(C(=O)O)cc3)cc2F)C1]

A mixture of methyl 4-(5-fluoro-6-((2R)-4-methylpyrrolidin-2-yl)pyridin-3-yl)benzoate (130 mg, 0.414 mmol) and lithium hydroxide monohydrate (104 mg, 2.48 mmol) in tetrahydrofuran (2.0 mL) and water (2.0 mL) was stirred at room temperature for 1 hour. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford 4-(5-fluoro-6-((2R)-4-methylpyrrolidin-2-yl)pyridin-3-yl)benzoic acid (115 mg, 93%) as a yellow-brown semi-solid. LCMS (ESI) [M+H]⁺: 301.3

### Step 3: 4-(6-((2R)-1-((2,2-difluorobenzo[d][1,3]dioxol-5-yl)carbamoyl)-4-methylpyrrolidin-2-yl)-5-fluoropyridin-3-yl)benzoic acid

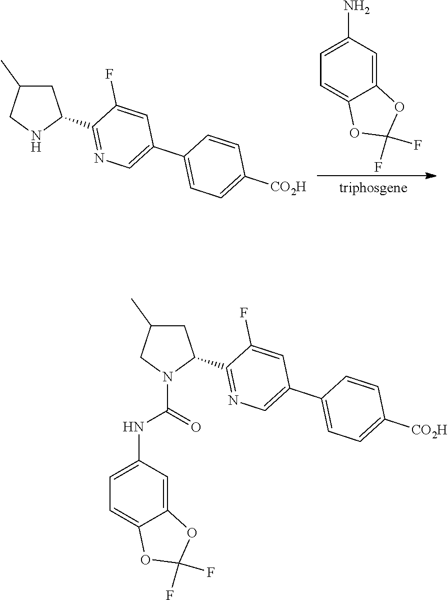 [SMILES: CC1CN[C@@H](c2ncc(-c3ccc(C(=O)O)cc3)cc2F)C1 ; Nc1ccc2c(c1)OC(F)(F)O2 ; CC1C[C@H](c2ncc(-c3ccc(C(=O)O)cc3)cc2F)N(C(=O)Nc2ccc3c(c2)OC(F)(F)O3)C1]

A solution of 4-(5-fluoro-6-((2R)-4-methylpyrrolidin-2-yl)pyridin-3-yl)benzoic acid (104 mg, 0.347 mmol), triphosgene (41.1 mg, 0.139 mmol), 2,2-difluoro-1,3-benzodioxol-5-amine (60.0 mg, 0.347 mmol), and N,N-diisopropylethylamine (224 mg, 1.74 mmol) in dichloromethane (3.0 mL) was stirred at room temperature for 1 hour. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 40 min; UV 254 nm) to give 4-(6-((2R)-1-((2,2-difluorobenzo[d][1,3]dioxol-5-yl)carbamoyl)-4-methylpyrrolidin-2-yl)-5-fluoropyridin-3-yl)benzoic acid (45 mg, 26%) as a yellow oil. LCMS (ESI) [M+H]⁺: 500.4

### Step 4: 4-(6-((2R,4R)-1-((2,2-difluorobenzo[d][1,3]dioxol-5-yl)carbamoyl)-4-methylpyrrolidin-2-yl)-5-fluoropyridin-3-yl)benzoic acid and 4-(6-((2R,4S)-1-((2,2-difluorobenzo[d][1,3]dioxol-5-yl)carbamoyl)-4-methylpyrrolidin-2-yl)-5-fluoropyridin-3-yl)benzoic acid

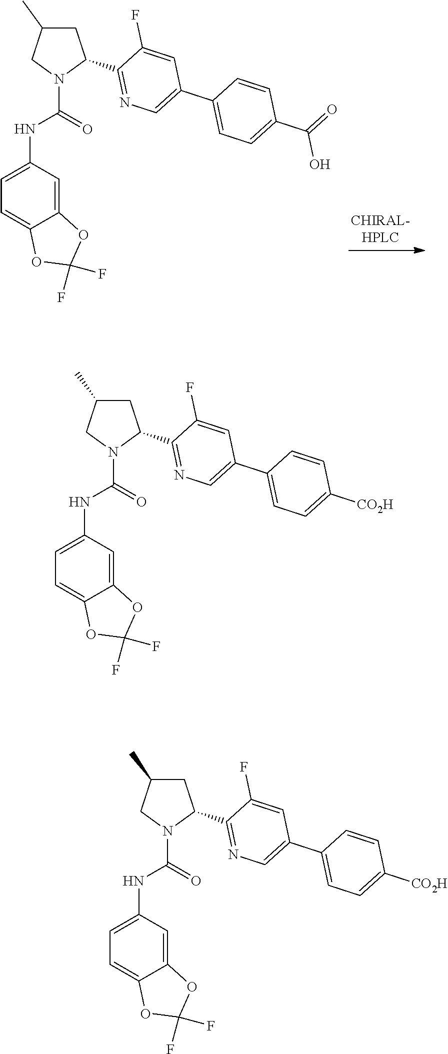 [SMILES: CC1C[C@H](c2ncc(-c3ccc(C(=O)O)cc3)cc2F)N(C(=O)Nc2ccc3c(c2)OC(F)(F)O3)C1 ; C[C@@H]1C[C@H](c2ncc(-c3ccc(C(=O)O)cc3)cc2F)N(C(=O)Nc2ccc3c(c2)OC(F)(F)O3)C1 ; C[C@H]1C[C@H](c2ncc(-c3ccc(C(=O)O)cc3)cc2F)N(C(=O)Nc2ccc3c(c2)OC(F)(F)O3)C1]

4-(6-((2R)-1-((2,2-Difluorobenzo[d][1,3]dioxol-5-yl)carbamoyl)-4-methylpyrrolidin-2-yl)-5-fluoropyridin-3-yl)benzoic acid (42.0 mg) was resolved by prep chiral-HPLC (conditions: column: CHIRALPAK ID, 3*25 cm, 5 μm; mobile phase A: MTBE/hexane (0.1% formic acid); 1:1; mobile phase B: ethanol; flow rate: 40 mL/min; gradient (B %): isocratic 10% B; detector: 220/254 nm; RT1 (min): 10.4; RT2 (min): 13.1) to afford (Compound 168a; Method AU, Peak 1, 1.16 min) 4-(6-((2R,4R)-1-((2,2-difluorobenzo[d][1,3]dioxol-5-yl)carbamoyl)-4-methylpyrrolidin-2-yl)-5-fluoropyridin-3-yl)benzoic acid (1^{st} peak, 4.3 mg, 10.2%) as a white solid and (Compound 168b; Method AU, Peak 2, 1.46 min) 4-(6-((2R,4S)-1-((2,2-difluorobenzo[d][1,3]dioxol-5-yl)carbamoyl)-4-methylpyrrolidin-2-yl)-5-fluoropyridin-3-yl)benzoic acid (2^{nd} peak, 11.6 mg, 28%) as a white solid.

| Cmpd # | Characterization Data |
|---|---|
| 168a | LCMS (ESI) [M + H]⁺: 500.2 |
| | ¹H NMR (400 MHz, DMSO-d₆) δ 8.67 (s, 1H), 8.46 (s, 1H), 7.96 (d, J = 8.0 Hz, 3H), 7.81 (d, |
| | J = 8.2 Hz, 2H), 7.46 (d, J = 2.0 Hz, 1H), 7.19-7.04 (m, 2H), 5.19 (dd, J = 9.5, 6.8 Hz, 1H), |
| | 3.82 (t, J = 8.3 Hz, 1H), 3.19-3.17 (m, 1H), 2.39-2.34 (m, 1H), 1.64-1.42 (m, 2H), 1.04 (d, |
| | J = 6.2 Hz, 3H). |
| 168b | LCMS (ESI) [M + H]⁺: 500.2 |
| | ¹H NMR (400 MHz, DMSO-d₆) δ 13.08 (s, 1H), 8.73 (s, 1H), 8.45 (s, 1H), 8.08 (dd, J = 11.4, |
| | 1.9 Hz, 1H), 8.06-7.97 (m, 2H), 7.89 (d, J = 8.2 Hz, 2H), 7.60 (s, 1H), 7.30-7.12 (m, 2H), |
| | 5.46-5.30 (m, 1H), 3.94 (t, J = 8.5 Hz, 1H), 3.18 (t, J = 8.7 Hz, 1H), 2.71-2.58 (m, 1H), |
| | 2.11-1.93 (m, 2H), 1.09 (d, J = 6.6 Hz, 3H). |

### Example 169: 4-(6-((2R,4S)-1-((2,2-difluoro-[1,3]dioxolo[4,5-b]pyridin-5-yl)carbamoyl)-4-methylpyrrolidin-2-yl)-5-fluoropyridin-3-yl)benzoic acid and 4-(6-((2R,4R)-1-((2,2-difluoro-[1,3]dioxolo[4,5-b]pyridin-5-yl)carbamoyl)-4-methylpyrrolidin-2-yl)-5-fluoropyridin-3-yl)benzoic acid

### Step 1: tert-butyl (2R)-2-(3-fluoro-5-(4-(methoxycarbonyl)phenyl)pyridin-2-yl)-4-methylpyrrolidine-1-carboxylate

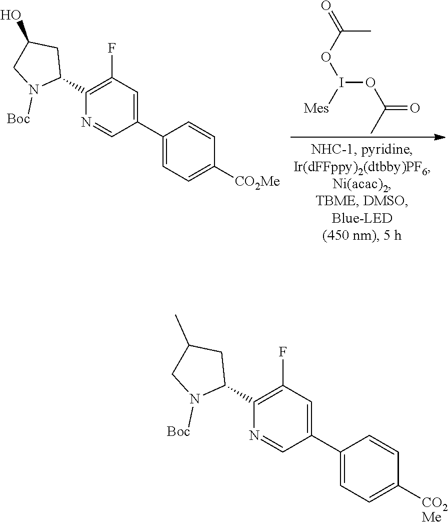

A mixture of tert-butyl (2R,4S)-2-(3-fluoro-5-(4-(methoxycarbonyl)phenyl)pyridin-2-yl)-4-hydroxypyrrolidine-1-carboxylate (1.30 g, 3.12 mmol), [acetoxy-(2,4,6-trimethylphenyl)-iodanyl]acetate (2.20 g, 6.24 mmol), 5,7-Di-tert-butyl-3-phenylbenzo[d]oxazol-3-ium tetrafluoroborate (NHC-1, 1.20 g, 4.05 mmol), pyridine (321 mg, 4.05 mmol), Ir(dFFppy)₂(dtbbpy)PF₆ (47.0 mg, 0.040 mmol) and Ni(acac)₂ (80.0 mg, 0.310 mmol) in DMSO (10.0 mL) and MTBE (10.0 mL) at room temperature was stirred under 450 nM blue LED irradiation for 5 hours. Upon completion, the mixture was diluted with water (50 mL), and the aqueous layer was extracted with ethyl acetate (5×20 mL). The combined organic layers were concentrated under reduced pressure. The crude residue was purified by silica gel column chromatography, eluted with petroleum ether/ethyl acetate (4:1) to afford tert-butyl (2R)-2-(3-fluoro-5-(4-(methoxycarbonyl)phenyl)pyridin-2-yl)-4-methylpyrrolidine-1-carboxylate (750 mg, 58%) as a yellow oil. LCMS (ESI) [M+H]⁺: 415.2

### Step 2: Methyl 4-(5-fluoro-6-((2R)-4-methylpyrrolidin-2-yl)pyridin-3-yl)benzoate

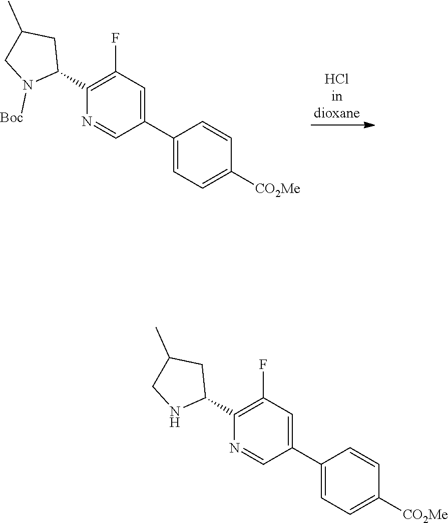 [SMILES: COC(=O)c1ccc(-c2cnc([C@H]3CC(C)CN3C(=O)OC(C)(C)C)c(F)c2)cc1 ; COC(=O)c1ccc(-c2cnc([C@H]3CC(C)CN3)c(F)c2)cc1]

To a solution of tert-butyl (2R)-2-(3-fluoro-5-(4-(methoxycarbonyl)phenyl)pyridin-2-yl)-4-methylpyrrolidine-1-carboxylate (750 mg, 1.64 mmol) in 1,4-dioxane (10.0 mL) was added HCl (4 mL, 16.0 mmol, 4.0 M in 1,4-dioxane), and the resultant mixture was stirred at room temperature for 2 hours. Upon completion, the resultant mixture was concentrated under reduced pressure to afford methyl 4-(5-fluoro-6-((2R)-4-methylpyrrolidin-2-yl)pyridin-3-yl)benzoate (550 mg, crude) as a yellow oil, which was used directly in the next step without further purification. LCMS (ESI) [M+H]⁺: 315.1

### Step 3: methyl 4-(6-((2R)-1-((2,2-difluoro-[1,3]dioxolo[4,5-b]pyridin-5-yl)carbamoyl)-4-methylpyrrolidin-2-yl)-5-fluoropyridin-3-yl)benzoate

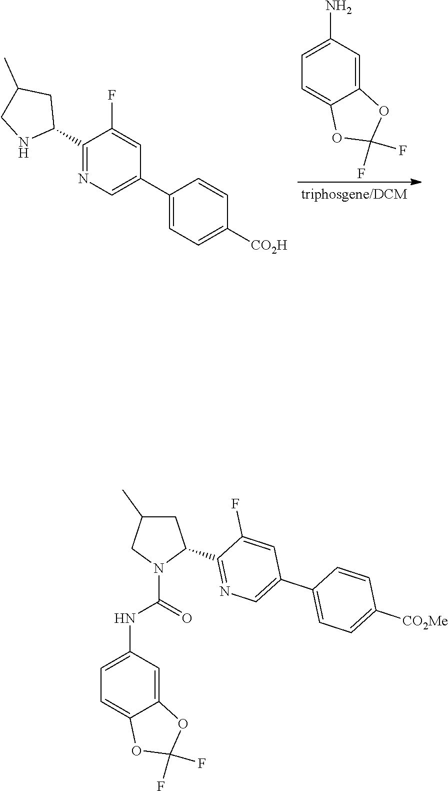 [SMILES: CC1CN[C@@H](c2ncc(-c3ccc(C(=O)O)cc3)cc2F)C1 ; Nc1ccc2c(c1)OC(F)(F)O2 ; COC(=O)c1ccc(-c2cnc([C@H]3CC(C)CN3C(=O)Nc3ccc4c(c3)OC(F)(F)O4)c(F)c2)cc1]

To a solution of 2,2-difluoro-[1,3]dioxolo[4,5-b]pyridin-5-amine (60.0 mg, 0.340 mmol) and methyl 4-(5-fluoro-6-((2R)-4-methylpyrrolidin-2-yl)pyridin-3-yl)benzoate (108 mg, 0.340 mmol), in dichloromethane (2.0 mL) at room temperature was added triphosgene (13.0 mg, 0.130 mmol) and DIEA (222 mg, 1.72 mmol), and the mixture was stirred for 1 hour. Upon completion, the mixture was concentrated under reduced pressure. The crude residue was directly purified by silica gel column chromatography, eluted with petroleum ether/ethyl acetate (6:1) to afford methyl 4-(6-((2R)-1-((2,2-difluoro-[1,3]dioxolo[4,5-b]pyridin-5-yl)carbamoyl)-4-methylpyrrolidin-2-yl)-5-fluoropyridin-3-yl)benzoate (50.0 mg, 27%) as a white solid. LCMS (ESI) [M+H]⁺: 515.1

### Step 4: 4-(6-((2R)-1-((2,2-difluoro-[1,3]dioxolo[4,5-b]pyridin-5-yl)carbamoyl)-4-methylpyrrolidin-2-yl)-5-fluoropyridin-3-yl)benzoic acid

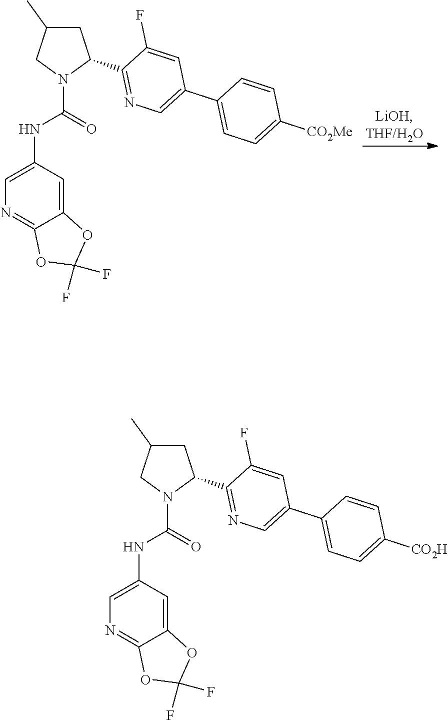 [SMILES: COC(=O)c1ccc(-c2cnc([C@H]3CC(C)CN3C(=O)Nc3cnc4c(c3)OC(F)(F)O4)c(F)c2)cc1 ; CC1C[C@H](c2ncc(-c3ccc(C(=O)O)cc3)cc2F)N(C(=O)Nc2cnc3c(c2)OC(F)(F)O3)C1]

To a mixture of methyl 4-(6-((2R)-1-((2,2-difluoro-[1,3]dioxolo[4,5-b]pyridin-5-yl)carbamoyl)-4-methylpyrrolidin-2-yl)-5-fluoropyridin-3-yl)benzoate (50.0 mg, 0.120 mmol) in water (0.50 mL) and THF (3.0 mL) was added lithium hydroxide monohydrate (52.9 mg, 1.26 mmol), and the mixture was stirred at room temperature overnight. Upon completion, the mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford 4-(6-((2R)-1-((2,2-difluoro-[1,3]dioxolo[4,5-b]pyridin-5-yl)carbamoyl)-4-methylpyrrolidin-2-yl)-5-fluoropyridin-3-yl)benzoic acid (50.0 mg, 79%) as a white solid. LCMS (ESI) [M+H]⁺: 500.1

### Step 5: 4-(6-((2R,4S)-1-((2,2-difluoro-[1,3]dioxolo[4,5-b]pyridin-5-yl)carbamoyl)-4-methylpyrrolidin-2-yl)-5-fluoropyridin-3-yl)benzoic acid and 4-(6-((2R,4R)-1-((2,2-difluoro-[1,3]dioxolo[4,5-b]pyridin-5-yl)carbamoyl)-4-methylpyrrolidin-2-yl)-5-fluoropyridin-3-yl)benzoic acid

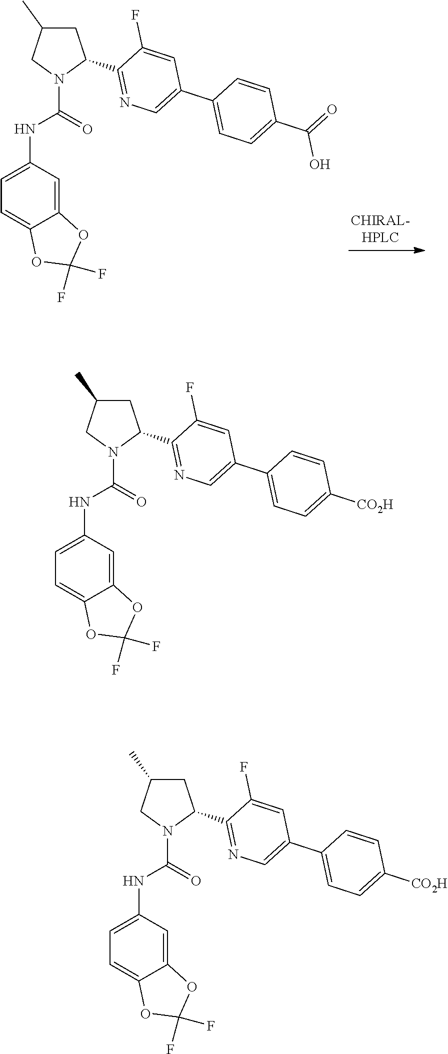 [SMILES: CC1C[C@H](c2ncc(-c3ccc(C(=O)O)cc3)cc2F)N(C(=O)Nc2ccc3c(c2)OC(F)(F)O3)C1 ; C[C@H]1C[C@H](c2ncc(-c3ccc(C(=O)O)cc3)cc2F)N(C(=O)Nc2ccc3c(c2)OC(F)(F)O3)C1 ; C[C@@H]1C[C@H](c2ncc(-c3ccc(C(=O)O)cc3)cc2F)N(C(=O)Nc2ccc3c(c2)OC(F)(F)O3)C1]

4-(6-((2R)-1-((2,2-Difluoro-[1,3]dioxolo[4,5-b]pyridin-5-yl)carbamoyl)-4-methylpyrrolidin-2-yl)-5-fluoropyridin-3-yl)benzoic acid (50.0 mg) was resolved by prep chiral-HPLC (conditions: column: CHIRALPAK IA, 3*25 cm, 5 μm; mobile phase A: MTBE/hexane (0.1% formic acid) (1:1); mobile phase B: methanol; Flow rate: 40 mL/min; gradient (B %): isocratic 30% B; detector: 220/254 nm; RT1 (min): 12.4; RT2 (min): 17.5) to afford (Compound 169a; Method AV, Peak 2, 2.08 min) 4-(6-((2R,4S)-1-((2,2-difluoro-[1,3]dioxolo[4,5-b]pyridin-5-yl)carbamoyl)-4-methylpyrrolidin-2-yl)-5-fluoropyridin-3-yl)benzoic acid (1^{st} peak, 31.7 mg, 63%) as a white solid and (Compound 169b; Method AV, Peak 1, 1.55 min) 4-(6-((2R,4R)-1-((2,2-difluoro-[1,3]dioxolo[4,5-b]pyridin-5-yl)carbamoyl)-4-methylpyrrolidin-2-yl)-5-fluoropyridin-3-yl)benzoic acid (2^{nd} peak, 6.3 mg, 13%) as a white solid.

| Cmpd # | Characterization Data |
|---|---|
| 169a | LCMS (ESI) [M + H]⁺: 501.2 |
| | ¹H NMR (400 MHz, Methanol-d₄) δ 8.64 (s, 1H), 8.13 (d, J = 8.5 Hz, 2H), 7.92 (dd, J = 11.2, |
| | 1.9 Hz, 1H), 7.79 (d, J = 8.5 Hz, 2H), 7.57 (d, J = 8.7 Hz, 1H), 7.49 (d, J = 8.7 Hz, 1H), 5.57- |
| | 5.47 (m, 1H), 4.01 (dd, J = 9.3, 7.5 Hz, 1H), 3.28-3.23 (m, 1H), 2.74-2.58 (m, 1H), 2.16- |
| | 2.07 (m, 2H), 1.16 (d, J = 6.7 Hz, 3H). |
| 169b | LCMS (ESI) [M + H]⁺: 501.2 |
| | ¹H NMR (400 MHz, Methanol-d₄) δ 8.65 (s, 1H), 8.13 (d, J = 8.4 Hz, 2H), 7.90 (dd, J = 11.2, |
| | 1.9 Hz, 1H), 7.79 (d, J = 8.4 Hz, 2H), 7.54-7.40 (m, 2H), 5.44-5.36 (m, 1H), 3.96-3.90 (m, |
| | 1H), 3.42-3.35 (m, 1H), 2.64-2.48 (m, 2H), 1.77-1.68 (m, 1H), 1.18 (d, J = 6.3 Hz, 3H). |

### Example 170: (S)-4-(6-(5-((2,2-difluorobenzo[d][1,3]dioxol-5-yl)carbamoyl)-5-azaspiro[2.4]heptan-6-yl)-5-fluoropyridin-3-yl)benzoic acid and (R)-4-(6-(5-((2,2-difluorobenzo[d][1,3]dioxol-5-yl)carbamoyl)-5-azaspiro[2.4]heptan-6-yl)-5-fluoropyridin-3-yl)benzoic acid

### Step 1: tert-butyl 6-(3-fluoro-5-(4-(methoxycarbonyl)phenyl)pyridin-2-yl)-5-azaspiro[2.4]heptane-5-carboxylate

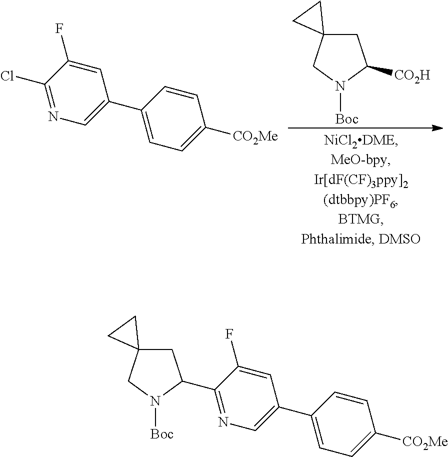

A solution of dichloronickel·1,2-dimethoxyethane (0.170 g, 0.753 mmol), phthalimide (1.11 g, 7.53 mmol), 4,4′-dimethoxy-2,2′-bipyridine (0.200 g, 0.903 mmol), Ir[dF(CF₃)ppy]₂(dtbbpy))PF₆ (0.170 g, 0.151 mmol), (S)-5-(tert-butoxycarbonyl)-5-azaspiro[2.4]heptane-6-carboxylic acid (2.00 g, 8.28 mmol), methyl 4-(6-chloro-5-fluoropyridin-3-yl)benzoate (2.00 g, 7.53 mmol), and N″-tert-butyl-N,N,N′,N′-tetramethylguanidine (1.93 g, 11.3 mmol) in DMSO (10.0 mL) at room temperature was stirred under 450 nm blue LED irradiation for 24 hours. Upon completion, the mixture was poured into water (50 mL) and extracted with ethyl acetate (10×10 mL). The combined organic layers were concentrated under reduced pressure. The crude residue was purified by silica gel column chromatography, eluted with petroleum ether/ethyl acetate (5:1) to afford tert-butyl 6-(3-fluoro-5-(4-(methoxycarbonyl)phenyl)pyridin-2-yl)-5-azaspiro[2.4]heptane-5-carboxylate (500 mg, 16%) as a yellow oil. LCMS (ESI) [M+H]⁺: 427.4

### Step 2: Methyl 4-(5-fluoro-6-(5-azaspiro[2.4]heptan-6-yl)pyridin-3-yl)benzoate

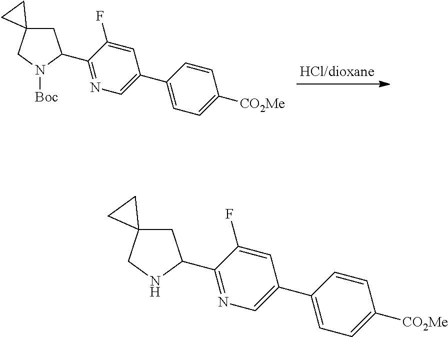 [SMILES: COC(=O)c1ccc(-c2cnc(C3CC4(CC4)CN3C(=O)OC(C)(C)C)c(F)c2)cc1 ; COC(=O)c1ccc(-c2cnc(C3CC4(CC4)CN3)c(F)c2)cc1]

To a solution of tert-butyl 6-(3-fluoro-5-(4-(methoxycarbonyl)phenyl)pyridin-2-yl)-5-azaspiro[2.4]heptane-5-carboxylate (500 mg, 1.17 mmol) in 1,4-dioxane (10.0 mL) at room temperature was added a solution of HCl (10.0 mL, 20.0 mmol, 2.0 M in 1,4-dioxane), and the resultant mixture was stirred for 1 hour. Upon completion, the mixture was concentrated under reduced pressure to afford methyl 4-(5-fluoro-6-(5-azaspiro[2.4]heptan-6-yl)pyridin-3-yl)benzoate (500 mg, crude) as a pink solid, which was used directly in the next step without further purification. LCMS (ESI) [M+H]⁺: 327.3

### Step 3: methyl 4-(6-(5-((2,2-difluorobenzo[d][1,3]dioxol-5-yl)carbamoyl)-5-azaspiro[2.4]heptan-6-yl)-5-fluoropyridin-3-yl)benzoate

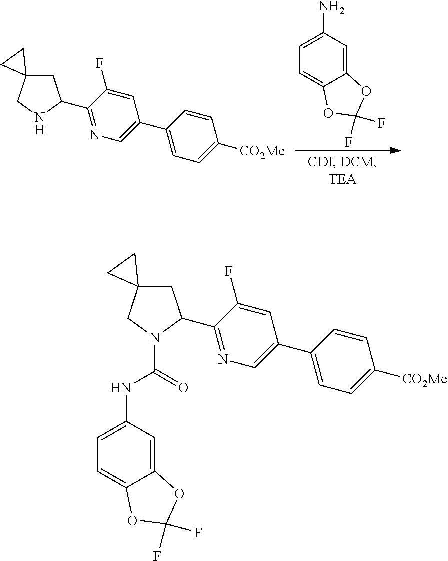 [SMILES: COC(=O)c1ccc(-c2cnc(C3CC4(CC4)CN3)c(F)c2)cc1 ; Nc1ccc2c(c1)OC(F)(F)O2 ; COC(=O)c1ccc(-c2cnc(C3CC4(CC4)CN3C(=O)Nc3ccc4c(c3)OC(F)(F)O4)c(F)c2)cc1]

A solution of 2,2-difluoro-1,3-benzodioxol-5-amine (119 mg, 0.690 mmol), carbonyldiimidazole (112 mg, 0.690 mmol), triethylamine (140 mg, 1.38 mmol), and methyl 4-(5-fluoro-6-(5-azaspiro[2.4]heptan-6-yl)pyridin-3-yl)benzoate (150 mg, 0.460 mmol) in dichloromethane (5.0 mL) was stirred at room temperature for 1 hour. Upon completion, the mixture was concentrated under reduced pressure. The crude residue was purified by silica gel column chromatography, eluted with petroleum ether/ethyl acetate (1:1) to afford methyl 4-(6-(5-((2,2-difluorobenzo[d][1,3]dioxol-5-yl)carbamoyl)-5-azaspiro[2.4]heptan-6-yl)-5-fluoropyridin-3-yl)benzoate (60.0 mg, 25%) as a white solid. LCMS (ESI) [M+H]⁺: 526.4

### Step 4: 4-(6-(5-((2,2-difluorobenzo[d][1,3]dioxol-5-yl)carbamoyl)-5-azaspiro[2.4]heptan-6-yl)-5-fluoropyridin-3-yl)benzoic acid

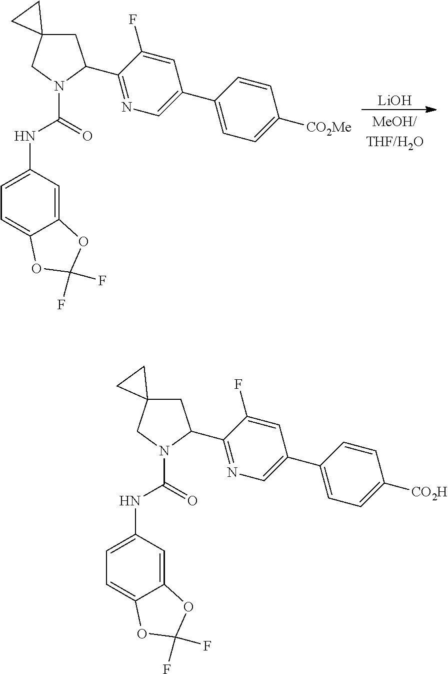 [SMILES: COC(=O)c1ccc(-c2cnc(C3CC4(CC4)CN3C(=O)Nc3ccc4c(c3)OC(F)(F)O4)c(F)c2)cc1 ; "LiOH" ; O=C(O)c1ccc(-c2cnc(C3CC4(CC4)CN3C(=O)Nc3ccc4c(c3)OC(F)(F)O4)c(F)c2)cc1]

A solution of methyl 4-(6-(5-((2,2-difluorobenzo[d][1,3]dioxol-5-yl)carbamoyl)-5-azaspiro[2.4]heptan-6-yl)-5-fluoropyridin-3-yl)benzoate (60.0 mg, 0.114 mmol) and lithium hydroxide monohydrate (23.9 mg, 0.570 mmol) in THF (2.0 mL), methanol (2 mL) and water (1 mL) was stirred at 50° C. for 1 hour. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford 4-(6-(5-((2,2-difluorobenzo[d][1,3]dioxol-5-yl)carbamoyl)-5-azaspiro[2.4]heptan-6-yl)-5-fluoropyridin-3-yl)benzoic acid (50 mg, 86%) as a white solid. LCMS (ESI) [M+H]⁺: 512.4

### Step 5: (S)-4-(6-(5-((2,2-difluorobenzo[d][1,3]dioxol-5-yl)carbamoyl)-5-azaspiro[2.4]heptan-6-yl)-5-fluoropyridin-3-yl)benzoic acid and (R)-4-(6-(5-((2,2-difluorobenzo[d][1,3]dioxol-5-yl)carbamoyl)-5-azaspiro[2.4]heptan-6-yl)-5-fluoropyridin-3-yl)benzoic acid

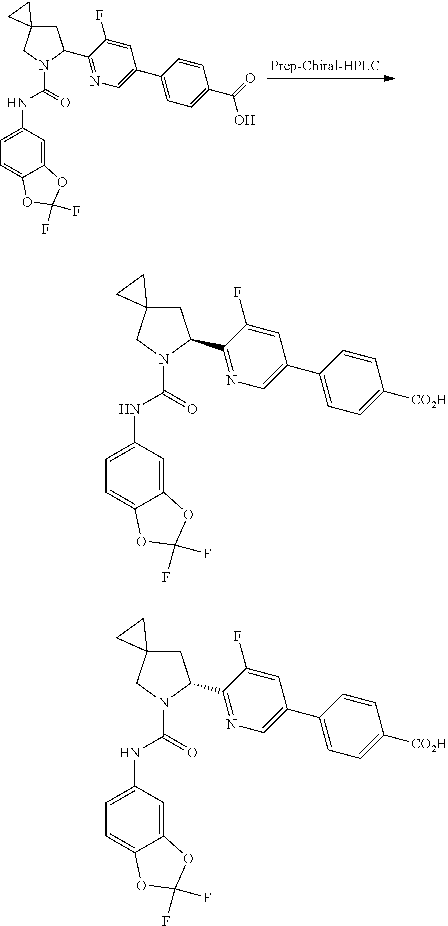 [SMILES: O=C(O)c1ccc(-c2cnc(C3CC4(CC4)CN3C(=O)Nc3ccc4c(c3)OC(F)(F)O4)c(F)c2)cc1 ; O=C(O)c1ccc(-c2cnc([C@@H]3CC4(CC4)CN3C(=O)Nc3ccc4c(c3)OC(F)(F)O4)c(F)c2)cc1 ; O=C(O)c1ccc(-c2cnc([C@H]3CC4(CC4)CN3C(=O)Nc3ccc4c(c3)OC(F)(F)O4)c(F)c2)cc1]

4-(6-(5-((2,2-Difluorobenzo[d][1,3]dioxol-5-yl)carbamoyl)-5-azaspiro[2.4]heptan-6-yl)-5-fluoropyridin-3-yl)benzoic acid (50.0 mg) was resolved by prep chiral-HPLC (conditions: Column: CHIRAL ART Cellulose-SC, 3*25 cm, 5 μm; Mobile Phase A: Hexane (0.1% FA), Mobile Phase B: EtOH; Flow rate: 40 mL/min; Gradient (B %): isocratic 12% B; Wave Length: 220/254 nm; RT1 (min): 12.9; RT2 (min): 16.7) to afford (Compound 170a; Method AW, Peak 1, 3.53 min) (S)-4-(6-(5-((2,2-difluorobenzo[d][1,3]dioxol-5-yl)carbamoyl)-5-azaspiro[2.4]heptan-6-yl)-5-fluoropyridin-3-yl)benzoic acid (1^{st} peak, 19.5 mg, 39%) as a white solid and (Compound 170b; Method AW, Peak 2, 4.73 min) (R)-4-(6-(5-((2,2-difluorobenzo[d][1,3]dioxol-5-yl)carbamoyl)-5-azaspiro[2.4]heptan-6-yl)-5-fluoropyridin-3-yl)benzoic acid (2^{nd} peak, 17.3 mg, 35%) as a white solid.

| Cmpd # | Characterization Data |
|---|---|
| 170a | LCMS (ESI) [M + H]⁺: 512.2 |
| | ¹H NMR (400 MHz, DMSO-d₆) δ 13.06 (s, 1H), 8.76 (s, 1H), 8.45 (s, 1H), 8.08 (dd, J = 11.5, |
| | 1.9 Hz, 1H), 8.03 (d, J = 8.0 Hz, 2H), 7.90 (d, J = 8.0 Hz, 2H), 7.58 (d, J = 2.0 Hz, 1H), 7.26- |
| | 7.08 (m, 2H), 5.45 (t, J = 6.6 Hz, 1H), 3.71 (d, J = 9.4 Hz, 1H), 3.56 (d, J = 9.5 Hz, 1H), 2.36 |
| | (dd, J = 12.6, 8.0 Hz, 1H), 1.92 (dd, J = 12.4, 5.2 Hz, 1H), 0.74-0.57 (m, 3H), 0.81 (s, 1H). |
| 170b | LCMS (ESI) [M + H]⁺: 512.3 |
| | ¹H NMR (400 MHz, DMSO-d₆) δ 13.00 (s, 1H), 8.76 (s, 1H), 8.44 (s, 1H), 8.10-7.99 (m, |
| | 3H), 7.90 (d, J = 8.0 Hz, 2H), 7.58 (d, J = 2.0 Hz, 1H), 7.25-7.12 (m, 2H), 5.54-5.34 (m, |
| | 1H), 3.71 (d, J = 9.4 Hz, 1H), 3.56 (d, J = 9.5 Hz, 1H), 2.37 (dd, J = 12.5, 7.9 Hz, 1H), 1.92 |
| | (dd, J = 12.4, 5.1 Hz, 1H), 0.72-0.53 (m, 3H), 0.69 (s, 1H). |

### Example 174: 4-(6-((2R,4R)-1-((2,2-difluoro-[1,3]dioxolo[4,5-b]pyridin-6-yl)carbamoyl)-4-methylpyrrolidin-2-yl)-5-fluoropyridin-3-yl)benzoic acid and 4-(6-((2R,4S)-1-((2,2-difluoro-[1,3]dioxolo[4,5-b]pyridin-6-yl)carbamoyl)-4-methylpyrrolidin-2-yl)-5-fluoropyridin-3-yl)benzoic acid

### Step 1: methyl 4-(6-((2R)-1-((2,2-difluoro-[1,3]dioxolo[4,5-b]pyridin-6-yl)carbamoyl)-4-methylpyrrolidin-2-yl)-5-fluoropyridin-3-yl)benzoate

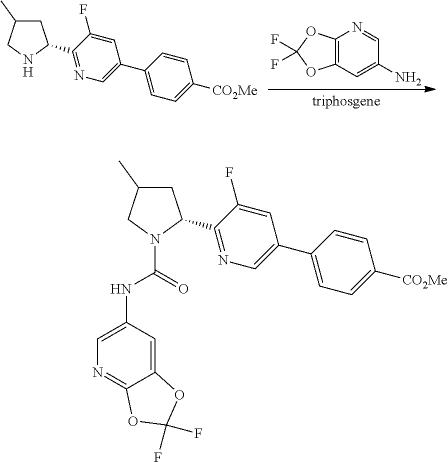 [SMILES: COC(=O)c1ccc(-c2cnc([C@H]3CC(C)CN3)c(F)c2)cc1 ; Nc1cnc2c(c1)OC(F)(F)O2 ; COC(=O)c1ccc(-c2cnc([C@H]3CC(C)CN3C(=O)Nc3cnc4c(c3)OC(F)(F)O4)c(F)c2)cc1]

To 2,2-difluoro-[1,3]dioxolo[4,5-b]pyridin-6-amine (50.0 mg, 0.287 mmol) in dichloromethane (5.0 mL) at 0° C. were added N, N-diisopropylethylamine (185 mg, 1.43 mmol) and triphosgene (34.0 mg, 0.115 mmol). After 30 minutes, the mixture was warmed to room temperature and methyl 4-(5-fluoro-6-((2R)-4-methylpyrrolidin-2-yl)pyridin-3-yl)benzoate (90.0 mg, 0.287 mmol) was added dropwise over 3 minutes. The reaction mixture was stirred for an additional 2 hours. Upon completion the reaction mixture was cooled in an ice bath and quenched with ice water. The mixture was extracted with ethyl acetate (3×8 mL). The combined organic layers were dried over anhydrous sodium sulfate, filtered, and concentrated under reduced pressure. The crude residue was purified by reversed-phase flash chromatography (conditions: column, C18 silica gel; mobile phase A: water (0.1% formic acid); mobile phase B: acetonitrile; gradient (B %): 0% to 100% over 25 min; detector: UV 254 nm) to afford methyl 4-(6-((2R)-1-((2,2-difluoro-[1,3]dioxolo[4,5-b]pyridin-6-yl)carbamoyl)-4-methylpyrrolidin-2-yl)-5-fluoropyridin-3-yl)benzoate (50 mg, 33% yield) as a yellow solid. LCMS (ESI) [M+H]⁺: 515.2

### Step 2: 4-(6-((2R)-1-((2,2-difluoro-[1,3]dioxolo[4,5-b]pyridin-6-yl)carbamoyl)-4-methylpyrrolidin-2-yl)-5-fluoropyridin-3-yl)benzoic acid

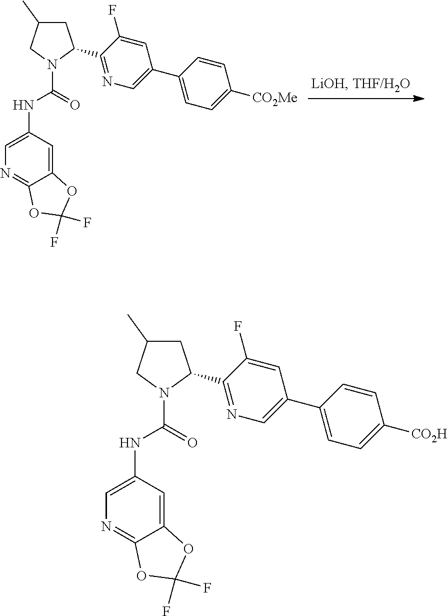 [SMILES: COC(=O)c1ccc(-c2cnc([C@H]3CC(C)CN3C(=O)Nc3cnc4c(c3)OC(F)(F)O4)c(F)c2)cc1 ; CC1C[C@H](c2ncc(-c3ccc(C(=O)O)cc3)cc2F)N(C(=O)Nc2cnc3c(c2)OC(F)(F)O3)C1]

To a solution of methyl 4-(6-((2R)-1-((2,2-difluoro-[1,3]dioxolo[4,5-b]pyridin-6-yl)carbamoyl)-4-methylpyrrolidin-2-yl)-5-fluoropyridin-3-yl)benzoate (45.0 mg, 87.0 μmol) in THF (1.5 mL) and water (1.5 mL) was added lithium hydroxide monohydrate (16.0 mg, 0.38 mmol) at 0° C. The resultant mixture was warmed to room temperature and stirred for 2 hours. Upon completion, the mixture was concentrated under reduced pressure. The crude residue was purified by reversed-phase flash chromatography (conditions: column, C18 silica gel; mobile phase A: water (0.1% formic acid); mobile phase B: acetonitrile; gradient (B %): 0% to 100% over 25 min; detector: UV 254 nm) to afford 4-(6-((2R)-1-((2,2-difluoro-[1,3]dioxolo[4,5-b]pyridin-6-yl)carbamoyl)-4-methylpyrrolidin-2-yl)-5-fluoropyridin-3-yl)benzoic acid (40.0 mg, 91%) as a white solid. LCMS (ESI) [M+H]⁺: 501.1

### Step 3: 4-(6-((2R,4R)-1-((2,2-difluoro-[1,3]dioxolo[4,5-b]pyridin-6-yl)carbamoyl)-4-methylpyrrolidin-2-yl)-5-fluoropyridin-3-yl)benzoic acid and 4-(6-((2R,4S)-1-((2,2-difluoro-[1,3]dioxolo[4,5-b]pyridin-6-yl)carbamoyl)-4-methylpyrrolidin-2-yl)-5-fluoropyridin-3-yl)benzoic acid

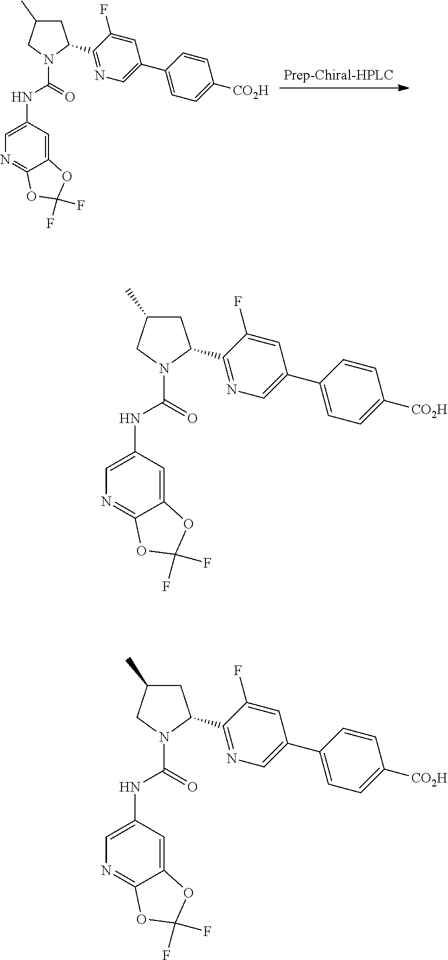 [SMILES: CC1C[C@H](c2ncc(-c3ccc(C(=O)O)cc3)cc2F)N(C(=O)Nc2cnc3c(c2)OC(F)(F)O3)C1 ; C[C@@H]1C[C@H](c2ncc(-c3ccc(C(=O)O)cc3)cc2F)N(C(=O)Nc2cnc3c(c2)OC(F)(F)O3)C1 ; C[C@H]1C[C@H](c2ncc(-c3ccc(C(=O)O)cc3)cc2F)N(C(=O)Nc2cnc3c(c2)OC(F)(F)O3)C1]

4-(6-((2R)-1-((2,2-Difluoro-[1,3]dioxolo[4,5-b]pyridin-6-yl)carbamoyl)-4-methylpyrrolidin-2-yl)-5-fluoropyridin-3-yl)benzoic acid (40.0 mg) was resolved by preparative chiral-HPLC (conditions: column: CHIRALPAK IA, 3*25 cm, 5 μm; mobile phase A: MTBE/hexane (0.1% formic acid) (1:1); mobile phase B: methanol; flow rate: 40 mL/min; gradient (B %): isocratic 30% B; detector: 220/254 nm; RT1 (min): 7.9; RT2 (min): 10.4) to afford first peak (Compound 174a; Method AV, Peak 1, 0.88 min) 4-(6-((2R,4R)-1-((2,2-difluoro-[1,3]dioxolo[4,5-b]pyridin-6-yl)carbamoyl)-4-methylpyrrolidin-2-yl)-5-fluoropyridin-3-yl)benzoic acid (5.5 mg, 13%) as a white solid, and second peak (Compound 174b; Method AV, Peak 2, 1.14 min) 4-(6-((2R,4S)-1-((2,2-difluoro-[1,3]dioxolo[4,5-b]pyridin-6-yl)carbamoyl)-4-methylpyrrolidin-2-yl)-5-fluoropyridin-3-yl)benzoic acid (20.5 mg, 51%) as a white solid.

| Compound # | Characterization Data |
|---|---|
| 174a | LCMS (ESI) [M + H]⁺: 501.2 |
| | ¹H NMR (400 MHz, DMSO-d₆) δ 13.07 (s, 1H), 8.75 (s, 1H), 8.05 (d, J = 9.6 Hz, 1H), 8.02- |
| | 7.98 (m, 2H), 7.97-7.95 (m, 2H), 7.89 (d, J = 8.4 Hz, 2H), 5.27 (t, J = 8.2 Hz, 1H), 3.92 (t, J = |
| | 7.8 Hz, 1H), 3.26-3.21 (m, 1H), 2.50-2.48 (m, 2H), 1.63-1.60 (m, 1H), 1.11 (d, J = 6.0 Hz, |
| | 3H). |
| 174b | LCMS (ESI) [M + H]⁺: 501.2 |
| | ¹H NMR (400 MHz, DMSO-d₆) δ 13.07 (s, 1H), 8.74 (s, 1H), 8.69 (s, 1H), 8.10-8.01 (m, 5H), |
| | 7.89 (d, J = 8.4 Hz, 2H), 5.41-5.39 (m, 1H), 3.94 (t, J = 8.4 Hz, 1H), 3.20 (t, J = 8.8 Hz, 1H), |
| | 2.67-2.638 (m, 1H), 2.08-2.01 (m, 2H), 1.09 (d, J = 6.4 Hz, 3H). |

### Example 187: (S)-5-(4-(1-((2,2-difluorobenzo[d][1,3]dioxol-5-yl)carbamoyl)-3,3-dimethylazetidine-2-carboxamido)phenyl)-6-methylpicolinic acid and (R)-5-(4-(1-((2,2-difluorobenzo[d][1,3]dioxol-5-yl)carbamoyl)-3,3-dimethylazetidine-2-carboxamido)phenyl)-6-methylpicolinic acid

### Step 1: methyl 3,3-dimethyl-2-(picolinamido)butanoate

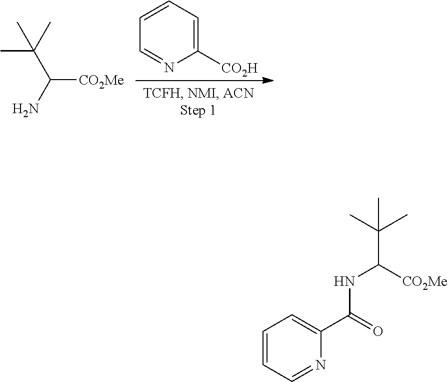

A solution of picolinic acid (5.51 g, 44.8 mmol), methyl 2-amino-3,3-dimethylbutanoate (5.00 g, 34.4 mmol), 1-methylimidazole (14.1 g, 172 mmol) and TCFH (14.5 g, 51.6 mmol) in acetonitrile (25.0 mL) at room temperature was stirred for 1 hour. Upon completion, the mixture was concentrated under reduced pressure. The crude residue was purified by silica gel column chromatography, eluted with petroleum ether/ethyl acetate (2:1) to afford methyl 3,3-dimethyl-2-(picolinamido)butanoate (7.2 g, 84%) as a colorless oil. LCMS (ESI) [M+H]⁺: 251.3.

### Step 2: methyl 3,3-dimethyl-1-picolinoylazetidine-2-carboxylate

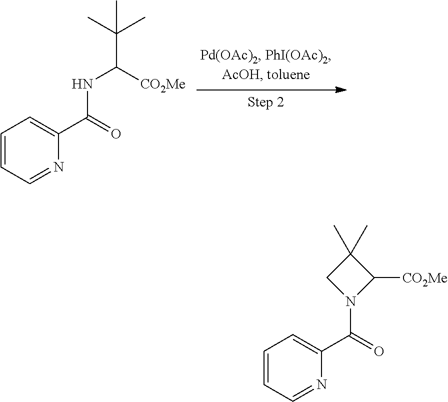 [SMILES: COC(=O)C(NC(=O)c1ccccn1)C(C)(C)C ; COC(=O)C1N(C(=O)c2ccccn2)CC1(C)C]

A solution of methyl 3,3-dimethyl-2-(pyridin-2-ylformamido)butanoate (7.20 g, 28.8 mmol) and (diacetoxyiodo)benzene (23.2 g, 71.9 mmol), acetic acid (3.45 g, 57.5 mmol) and palladium (II) acetate (323 mg, 1.44 mmol) in toluene (40.0 mL) was stirred at 110° C. for 2 hours. Upon completions, the resultant mixture was concentrated under reduced pressure. The crude residue was directly purified by silica gel column chromatography, eluted with petroleum ether/ethyl acetate (1:1), to afford methyl 3,3-dimethyl-1-picolinoylazetidine-2-carboxylate (6.50 g, 91%) as a yellow oil. LCMS (ESI) [M+H]⁺: 249.3.

### Step 3: 3,3-dimethylazetidine-2-carboxylic acid

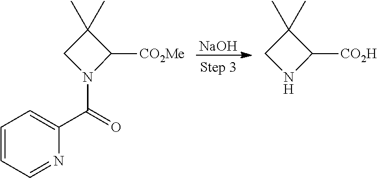

A solution of methyl 3,3-dimethyl-1-picolinoylazetidine-2-carboxylate (1.00 g, 4.03 mmol) and sodium hydroxide (1.61 g, 40.3 mmol) in methanol (4.0 mL), tetrahydrofuran (4.0 mL) and water (3.0 mL) was stirred at 50° C. for 4 hours. Upon completion, the mixture was concentrated under reduced pressure to afford crude 3,3-dimethylazetidine-2-carboxylic acid (800 mg, crude) as an off-white solid, which was used directly in the next step without further purification. LCMS (ESI) [M+H]⁺: 130.2.

### Step 4: 1-((2,2-difluorobenzo[d][1,3]dioxol-5-yl)carbamoyl)-3,3-dimethylazetidine-2-carboxylic acid

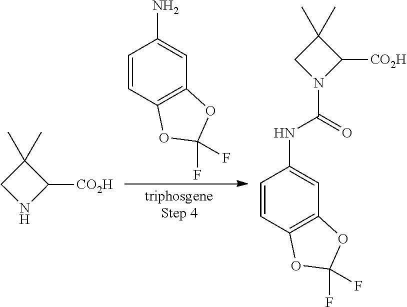

To a solution of 2,2-difluoro-1,3-benzodioxol-5-amine (250 mg, 1.44 mmol), N, N-diisopropylethylamine (933 mg, 7.22 mmol) and 3,3-dimethylazetidine-2-carboxylic acid (186 mg, 1.44 mmol) in dichloromethane (5.0 mL) at room temperature was added triphosgene (171 mg, 0.578 mmol). After 1 hour, the mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 20 min; detector: UV 254 nm) to afford 1-((2,2-difluorobenzo[d][1,3]dioxol-5-yl)carbamoyl)-3,3-dimethylazetidine-2-carboxylic acid (130 mg, 27%) as an off-white solid. LCMS (ESI) [M+H]⁺: 329.3.

### Step 5: methyl 5-(4-(1-((2,2-difluorobenzo[d][1,3]dioxol-5-yl)carbamoyl)-3,3-dimethylazetidine-2-carboxamido)phenyl)-6-methylpicolinate

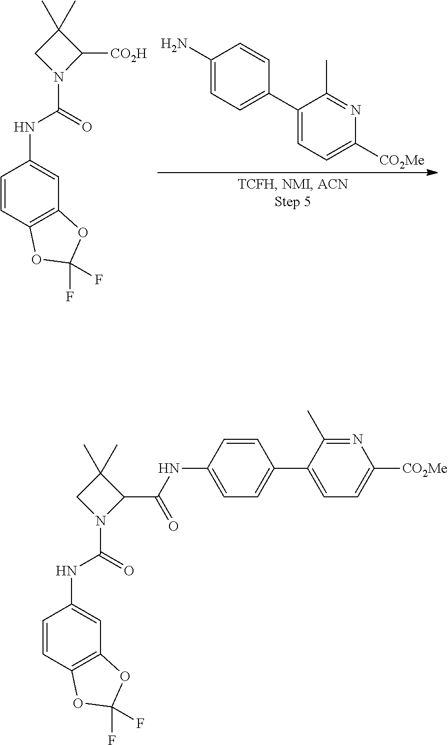 [SMILES: CC1(C)CN(C(=O)Nc2ccc3c(c2)OC(F)(F)O3)C1C(=O)O ; COC(=O)c1ccc(-c2ccc(N)cc2)c(C)n1 ; COC(=O)c1ccc(-c2ccc(NC(=O)C3N(C(=O)Nc4ccc5c(c4)OC(F)(F)O5)CC3(C)C)cc2)c(C)n1]

A solution of 1-((2,2-difluorobenzo[d][1,3]dioxol-5-yl)carbamoyl)-3,3-dimethylazetidine-2-carboxylic acid (130 mg, 0.396 mmol), TCFH (222 mg, 0.792 mmol), methyl 5-(4-aminophenyl)-6-methylpyridine-2-carboxylate (144 mg, 0.594 mmol) and 1-methylimidazole (163 mg, 1.98 mmol) in acetonitrile (3.0 mL) at room temperature was stirred for 1 hour. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford methyl 5-(4-(1-((2,2-difluorobenzo[d][1,3]dioxol-5-yl)carbamoyl)-3,3-dimethylazetidine-2-carboxamido)phenyl)-6-methylpicolinate (120 mg, 55%) as a tan solid. LCMS (ESI) [M+H]⁺: 554.0.

### Step 6: 5-(4-(1-((2,2-difluorobenzo[d][1,3]dioxol-5-yl)carbamoyl)-3,3-dimethylazetidine-2-carboxamido)phenyl)-6-methylpicolinic acid

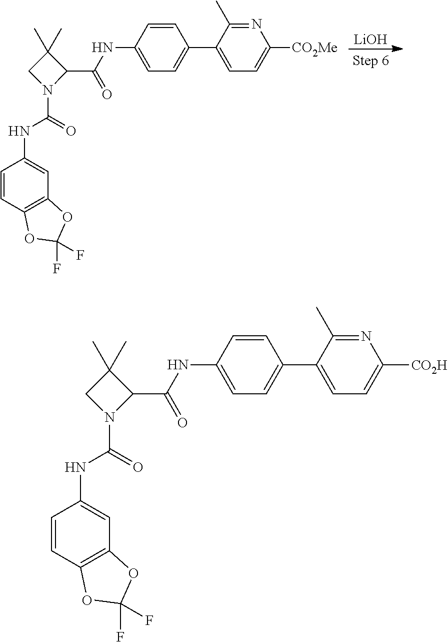 [SMILES: COC(=O)c1ccc(-c2ccc(NC(=O)C3N(C(=O)Nc4ccc5c(c4)OC(F)(F)O5)CC3(C)C)cc2)c(C)n1 ; "LiOH" ; Cc1nc(C(=O)O)ccc1-c1ccc(NC(=O)C2N(C(=O)Nc3ccc4c(c3)OC(F)(F)O4)CC2(C)C)cc1]

To a solution of methyl 5-(4-(1-((2,2-difluorobenzo[d][1,3]dioxol-5-yl)carbamoyl)-3,3-dimethylazetidine-2-carboxamido)phenyl)-6-methylpicolinate (110 mg, 0.199 mmol) in tetrahydrofuran (2.5 mL) and water (2.0 mL) at room temperature was added lithium hydroxide monohydrate (50.0 mg, 1.19 mmol). After 1 hour, the reaction mixture was directly purified by reversed-phase column chromatography (conditions: column: C18 silica gel; mobile phase A: water (0.1% formic acid); mobile phase B: acetonitrile; gradient (B %): 0% to 100% over 25 min; detector: UV 254 nm) to afford 5-(4-(1-((2,2-difluorobenzo[d][1,3]dioxol-5-yl)carbamoyl)-3,3-dimethylazetidine-2-carboxamido)phenyl)-6-methylpicolinic acid (70.0 mg, 65%) as a white solid. LCMS (ESI) [M+H]⁺: 539.5.

### Step 7: (S)-5-(4-(1-((2,2-difluorobenzo[d][1,3]dioxol-5-yl)carbamoyl)-3,3-dimethylazetidine-2-carboxamido)phenyl)-6-methylpicolinic acid and (R)-5-(4-(1-((2,2-difluorobenzo[d][1,3]dioxol-5-yl)carbamoyl)-3,3-dimethylazetidine-2-carboxamido)phenyl)-6-methylpicolinic acid

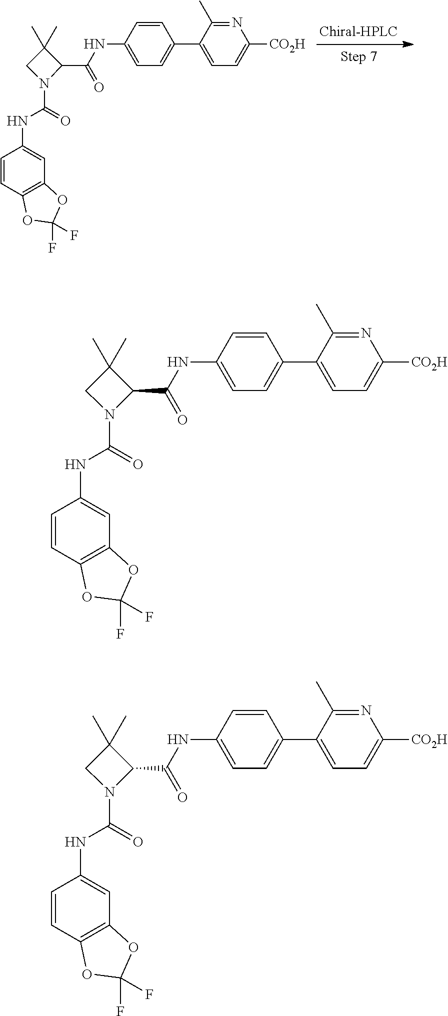 [SMILES: Cc1nc(C(=O)O)ccc1-c1ccc(NC(=O)C2N(C(=O)Nc3ccc4c(c3)OC(F)(F)O4)CC2(C)C)cc1 ; Cc1nc(C(=O)O)ccc1-c1ccc(NC(=O)[C@H]2N(C(=O)Nc3ccc4c(c3)OC(F)(F)O4)CC2(C)C)cc1 ; Cc1nc(C(=O)O)ccc1-c1ccc(NC(=O)[C@@H]2N(C(=O)Nc3ccc4c(c3)OC(F)(F)O4)CC2(C)C)cc1]

5-(4-(1-((2,2-difluorobenzo[d][1,3]dioxol-5-yl)carbamoyl)-3,3-dimethylazetidine-2-carboxamido)phenyl)-6-methylpicolinic acid (70 mg) was resolved by preparative chiral HPLC (conditions: column: CHIRAL ART Cellulose-SB, 3*25 cm, 5 μm; mobile phase A: hexane (0.1% formic acid), mobile phase B: ethanol; flow rate: 40 mL/min; Gradient (B %): isocratic 50% B; detector: 220/254 nm; RT1 (min): 6.5; RT2 (min): 13.0) to afford (Compound 187a; Method AY, Peak 1, 1.16 min) (S)-5-(4-(1-((2,2-difluorobenzo[d][1,3]dioxol-5-yl)carbamoyl)-3,3-dimethylazetidine-2-carboxamido)phenyl)-6-methylpicolinic acid (1^{st} peak, 15.7 mg, 24%) as a white solid and (Compound 187b; Method AY, Peak 2, 2.50 min) (R)-5-(4-(1-((2,2-difluorobenzo[d][1,3]dioxol-5-yl)carbamoyl)-3,3-dimethylazetidine-2-carboxamido)phenyl)-6-methylpicolinic acid (2^{nd} peak, 19.6 mg, 30%) as a white solid.

| Cmpd # | Characterization Data |
|---|---|
| 187a | LCMS (ESI) [M + H]⁺: 539.2 |
| | ¹H NMR (400 MHz, DMSO-d₆) δ 10.03 (s, 1H), 8.77 (s, 1H), 7.91 (dd, J = 8.3, 3.0 Hz, 1H), 7.76 |
| | (dd, J = 13.3, 7.2 Hz, 3H), 7.67 (t, J = 1.8 Hz, 1H), 7.41 (d, J = 8.2 Hz, 2H), 7.32-7.19 (m, 2H), |
| | 4.42 (d, J = 2.4 Hz, 1H), 3.76 (d, J = 7.4 Hz, 1H), 3.68 (d, J = 7.3 Hz, 1H), 2.49 (s, 3H), 1.41 (s, |
| | 3H), 1.14 (s, 3H). |
| 187b | LCMS (ESI) [M + H]⁺: 539.3 |
| | ¹H NMR (400 MHz, DMSO-d₆) δ 10.03 (s, 1H), 8.78 (d, J = 5.5 Hz, 1H), 7.88 (s, 1H), 7.77 (d, J = |
| | 8.1 Hz, 2H), 7.73 (d, J = 7.7 Hz, 1H), 7.67 (d, J = 2.0 Hz, 1H), 7.40 (d, J = 8.2 Hz, 2H), 7.28 (d, |
| | J = 8.8 Hz, 1H), 7.25-7.18 (m, 1H), 4.42 (s, 1H), 3.76 (d, J = 7.4 Hz, 1H), 3.68 (d, J = 7.4 Hz, 1H), |
| | 2.49 (s, 3H), 1.41 (s, 3H), 1.14 (s, 3H). |

### Example 188: (S)-5-(4-(1-((5-fluoro-6-(1-(trifluoromethyl)cyclopropyl)pyridin-3-yl)carbamoyl)-3,3-dimethylazetidine-2-carboxamido)phenyl)-6-methylpicolinic acid and (R)-5-(4-(1-((5-fluoro-6-(1-(trifluoromethyl)cyclopropyl)pyridin-3-yl)carbamoyl)-3,3-dimethylazetidine-2-carboxamido)phenyl)-6-methylpicolinic acid

### Step 1: 1-((5-fluoro-6-(1-(trifluoromethyl)cyclopropyl)pyridin-3-yl)carbamoyl)-3,3-dimethylazetidine-2-carboxylic acid

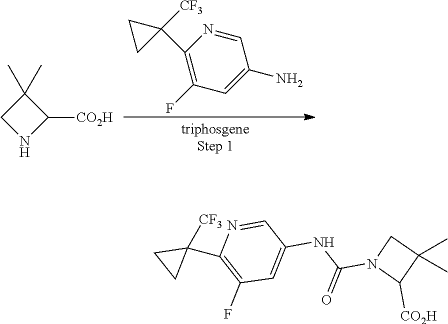

A solution of 5-fluoro-6-(1-(trifluoromethyl)cyclopropyl)pyridin-3-amine (90.0 mg, 0.409 mmol), N, N-diisopropylethylamine (264 mg, 2.04 mmol), 3,3-dimethylazetidine-2-carboxylic acid (211 mg, 1.64 mmol) and triphosgene (48.5 mg, 0.164 mmol) in dichloromethane (5.0 mL) was stirred at room temperature for 1 hour. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford 1-((5-fluoro-6-(1-(trifluoromethyl)cyclopropyl)pyridin-3-yl)carbamoyl)-3,3-dimethylazetidine-2-carboxylic acid (85.0 mg, 55%) as an off-white solid. LCMS (ESI) [M+H]⁺: 376.1.

### Step 2: methyl 5-(4-(1-((5-fluoro-6-(1-(trifluoromethyl)cyclopropyl)pyridin-3-yl)carbamoyl)-3,3-dimethylazetidine-2-carboxamido)phenyl)-6-methylpicolinate

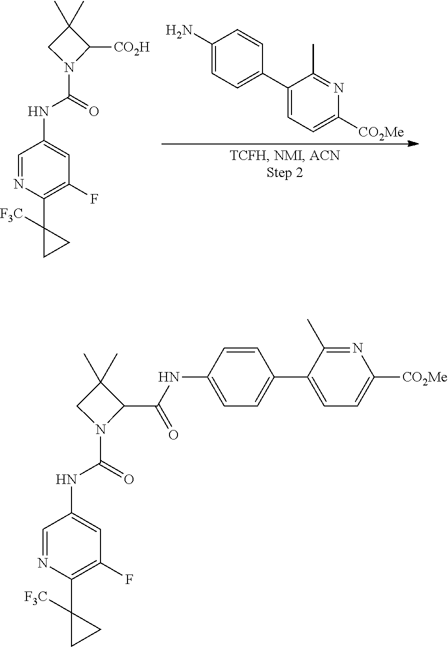 [SMILES: CC1(C)CN(C(=O)Nc2cnc(C3(C(F)(F)F)CC3)c(F)c2)C1C(=O)O ; COC(=O)c1ccc(-c2ccc(N)cc2)c(C)n1 ; COC(=O)c1ccc(-c2ccc(NC(=O)C3N(C(=O)Nc4cnc(C5(C(F)(F)F)CC5)c(F)c4)CC3(C)C)cc2)c(C)n1]

To a solution of 1-((5-fluoro-6-(1-(trifluoromethyl)cyclopropyl)pyridin-3-yl)carbamoyl)-3,3-dimethylazetidine-2-carboxylic acid (80.0 mg, 0.213 mmol), in acetonitrile (3.0 mL) at room temperature was added sequentially TCFH (120 mg, 0.426 mmol), methyl 5-(4-aminophenyl)-6-methylpyridine-2-carboxylate (62.0 mg, 0.256 mmol) and 1-methylimidazole (105 mg, 1.28 mmol). After 1 hour, the reaction mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 35 min; detector: UV 254 nm) to afford methyl 5-(4-(1-((5-fluoro-6-(1-(trifluoromethyl)cyclopropyl)pyridin-3-yl)carbamoyl)-3,3-dimethylazetidine-2-carboxamido)phenyl)-6-methylpicolinate (90 mg, 72%) as a tan solid. LCMS (ESI) [M+H]⁺: 600.6.

### Step 3: 5-(4-(1-((5-fluoro-6-(1-(trifluoromethyl)cyclopropyl)pyridin-3-yl)carbamoyl)-3,3-dimethylazetidine-2-carboxamido)phenyl)-6-methylpicolinic acid

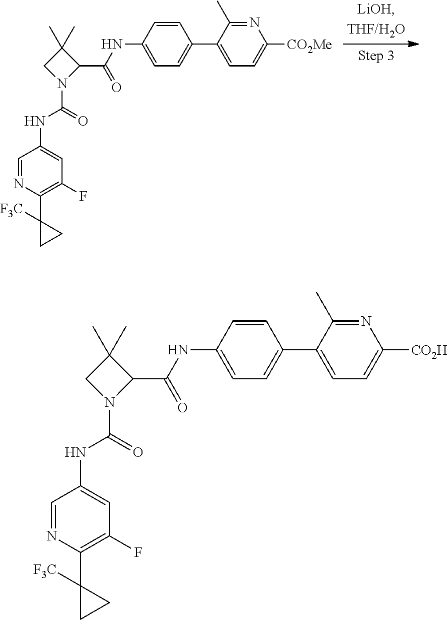 [SMILES: COC(=O)c1ccc(-c2ccc(NC(=O)C3N(C(=O)Nc4cnc(C5(C(F)(F)F)CC5)c(F)c4)CC3(C)C)cc2)c(C)n1 ; Cc1nc(C(=O)O)ccc1-c1ccc(NC(=O)C2N(C(=O)Nc3cnc(C4(C(F)(F)F)CC4)c(F)c3)CC2(C)C)cc1]

To a solution of methyl 5-(4-(1-((5-fluoro-6-(1-(trifluoromethyl)cyclopropyl)pyridin-3-yl)carbamoyl)-3,3-dimethylazetidine-2-carboxamido)phenyl)-6-methylpicolinate (60.0 mg, 0.100 mmol) in tetrahydrofuran (2.5 mL) and water (2.0 mL) at room temperature was added lithium hydroxide monohydrate (42.0 mg, 1.00 mmol). After 1 hour, the reaction mixture was directly purified by reversed-phase column chromatography (conditions: column: C18 silica gel; mobile phase A: water (0.1% formic acid); mobile phase B: acetonitrile; gradient (B %): 0% to 100% over 20 min; detector: UV 254 nm) to afford 5-(4-(1-((5-fluoro-6-(1-(trifluoromethyl)cyclopropyl)pyridin-3-yl)carbamoyl)-3,3-dimethylazetidine-2-carboxamido)phenyl)-6-methylpicolinic acid (35.0 mg, 60%) as a white solid. LCMS (ESI) [M+H]⁺: 586.6.

### Step 4: (S)-5-(4-(1-((5-fluoro-6-(1-(trifluoromethyl)cyclopropyl)pyridin-3-yl)carbamoyl)-3,3-dimethylazetidine-2-carboxamido)phenyl)-6-methylpicolinic acid and (R)-5-(4-(1-((5-fluoro-6-(1-(trifluoromethyl)cyclopropyl)pyridin-3-yl)carbamoyl)-3,3-dimethylazetidine-2-carboxamido)phenyl)-6-methylpicolinic acid

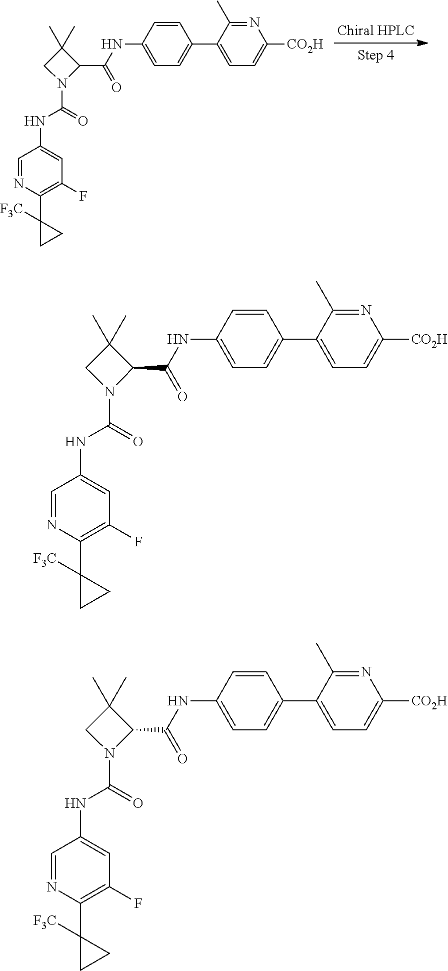 [SMILES: Cc1nc(C(=O)O)ccc1-c1ccc(NC(=O)C2N(C(=O)Nc3cnc(C4(C(F)(F)F)CC4)c(F)c3)CC2(C)C)cc1 ; Cc1nc(C(=O)O)ccc1-c1ccc(NC(=O)[C@H]2N(C(=O)Nc3cnc(C4(C(F)(F)F)CC4)c(F)c3)CC2(C)C)cc1 ; Cc1nc(C(=O)O)ccc1-c1ccc(NC(=O)[C@@H]2N(C(=O)Nc3cnc(C4(C(F)(F)F)CC4)c(F)c3)CC2(C)C)cc1]

5-(4-(1-((5-fluoro-6-(1-(trifluoromethyl)cyclopropyl)pyridin-3-yl)carbamoyl)-3,3-dimethylazetidine-2-carboxamido)phenyl)-6-methylpicolinic acid (32 mg) was resolved by preparative chiral HPLC (column: CHIRAL ART Cellulose-SB, 3*25 cm, 5 μm; mobile phase A: HEX:MTBE=1:1 (0.1% formic acid), mobile phase B: MeOH; flow rate: 40 mL/min; Gradient (B %): isocratic 20% B; detector: 220/254 nm; RT1 (min): 9.4; RT2 (min): 15.4) to afford (Compound 188a; Method AZ, Peak 1, 0.88 min) (S)-5-(4-(1-((5-fluoro-6-(1-(trifluoromethyl)cyclopropyl)pyridin-3-yl)carbamoyl)-3,3-dimethylazetidine-2-carboxamido)phenyl)-6-methylpicolinic acid (1^{st} peak, 9.8 mg, 31%) as a white solid and (Compound 188b; Method AZ, Peak 2, 1.53 min) (R)-5-(4-(1-((5-fluoro-6-(1-(trifluoromethyl)cyclopropyl)pyridin-3-yl)carbamoyl)-3,3-dimethylazetidine-2-carboxamido)phenyl)-6-methylpicolinic acid (2^{nd} peak, 8.7 mg, 27%) as a white solid.

| Cmpd # | Characterization Data |
|---|---|
| 188a | LCMS (ESI) [M + H]⁺: 586.2 |
| | ¹H NMR (400 MHz, DMSO-d₆) δ 10.09 (s, 1H), 9.17 (s, 1H), 8.51 (s, 1H), 7.98 (dd, J = 12.7, 2.1 |
| | Hz, 1H), 7.92 (d, J = 7.9 Hz, 1H), 7.81-7.72 (m, 3H), 7.41 (d, J = 8.2 Hz, 2H), 4.47 (s, 1H), 3.79 |
| | (d, J = 7.4 Hz, 1H), 3.71 (d, J = 7.4 Hz, 1H), 2.49 (s, 3H), 1.51-1.04 (m, 10H). |
| 188b | LCMS (ESI) [M + H]⁺: 586.2 |
| | ¹H NMR (400 MHz, DMSO-d₆) δ 10.09 (s, 1H), 9.18 (s, 1H), 8.51 (s, 1H), 7.98 (d, J = 12.6 Hz, |
| | 1H), 7.91 (d, J = 7.6 Hz, 1H), 7.76 (t, J = 8.7 Hz, 3H), 7.41 (d, J = 8.3 Hz, 2H), 4.47 (s, 1H), 3.79 |
| | (d, J = 7.4 Hz, 1H), 3.71 (d, J = 7.5 Hz, 1H), 2.49 (s, 3H), 1.54-1.33 (m, 5H), 1.22-1.05 (m, |
| | 5H). |

### Example 189: (S)-5-(4-(1-((2,2-difluoro-[1,3]dioxolo[4,5-b]pyridin-5-yl)carbamoyl)-3,3-dimethylazetidine-2-carboxamido)phenyl)-6-methylpicolinic acid and (R)-5-(4-(1-((2,2-difluoro-[1,3]dioxolo[4,5-b]pyridin-5-yl)carbamoyl)-3,3-dimethylazetidine-2-carboxamido)phenyl)-6-methylpicolinic acid

### Step 1: 1-((2,2-difluoro-[1,3]dioxolo[4,5-b]pyridin-5-yl)carbamoyl)-3,3-dimethylazetidine-2-carboxylic acid

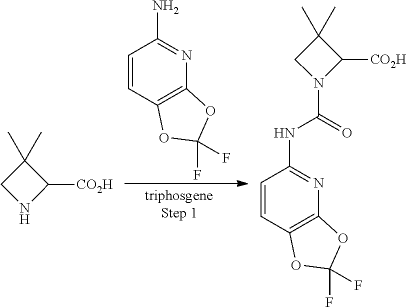

To a solution of 2,2-difluoro-[1,3]dioxolo[4,5-b]pyridin-5-amine (80 mg, 0.459 mmol) in dichloromethane (5.0 mL) at room temperature was added sequentially N, N-diisopropylethylamine (297 mg, 2.30 mmol), 3,3-dimethylazetidine-2-carboxylic acid (59.4 mg, 0.459 mmol) and triphosgene (54.5 mg, 0.184 mmol). After 1 hour, the reaction mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford 1-((2,2-difluoro-[1,3]dioxolo[4,5-b]pyridin-5-yl)carbamoyl)-3,3-dimethylazetidine-2-carboxylic acid (50.0 mg, 33%) as a yellow oil. LCMS (ESI) [M+H]⁺: 330.2.

### Step 2: methyl 5-(4-(1-((2,2-difluoro-[1,3]dioxolo[4,5-b]pyridin-5-yl)carbamoyl)-3,3-dimethylazetidine-2-carboxamido)phenyl)-6-methylpicolinate

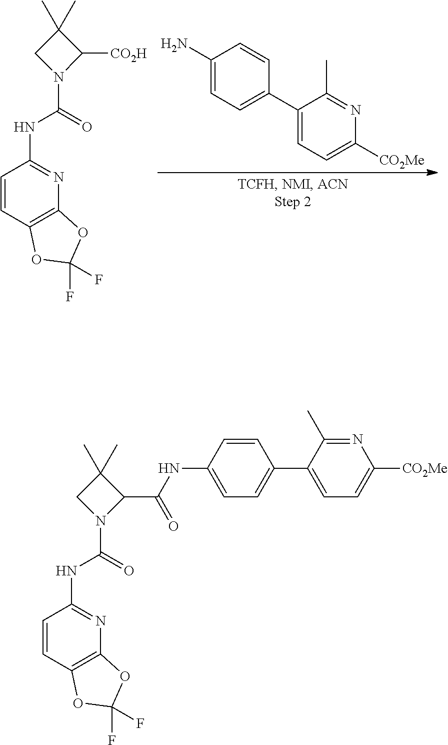 [SMILES: CC1(C)CN(C(=O)Nc2ccc3c(n2)OC(F)(F)O3)C1C(=O)O ; COC(=O)c1ccc(-c2ccc(N)cc2)c(C)n1 ; COC(=O)c1ccc(-c2ccc(NC(=O)C3N(C(=O)Nc4ccc5c(n4)OC(F)(F)O5)CC3(C)C)cc2)c(C)n1]

To a solution of 1-((2,2-difluoro-[1,3]dioxolo[4,5-b]pyridin-5-yl)carbamoyl)-3,3-dimethylazetidine-2-carboxylic acid (45.0 mg, 0.137 mmol) in acetonitrile (3.0 mL) at room temperature was added sequentially TCFH (76.7 mg, 0.274 mmol), methyl 5-(4-aminophenyl)-6-methylpyridine-2-carboxylate (49.7 mg, 0.206 mmol), and 1-methylimidazole (67.3 mg, 0.822 mmol). After 1 hour, the reaction mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford methyl 5-(4-(1-((2,2-difluoro-[1,3]dioxolo[4,5-b]pyridin-5-yl)carbamoyl)-3,3-dimethylazetidine-2-carboxamido)phenyl)-6-methylpicolinate (70.0 mg, 92%) as a tan solid. LCMS (ESI) [M+H]⁺: 554.5.

### Step 3: 5-(4-(1-((2,2-difluoro-[1,3]dioxolo[4,5-b]pyridin-5-yl)carbamoyl)-3,3-dimethylazetidine-2-carboxamido)phenyl)-6-methylpicolinic acid

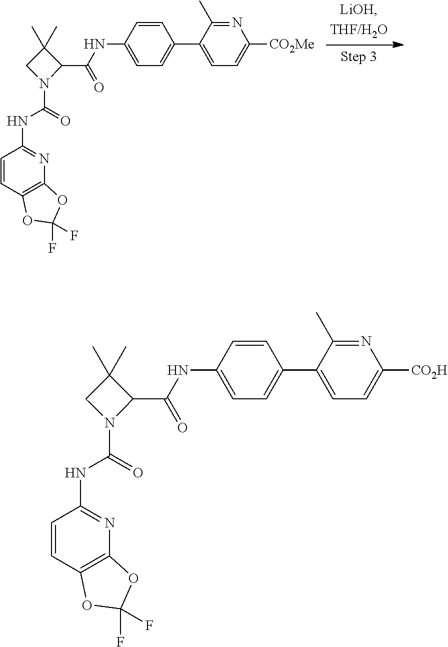 [SMILES: COC(=O)c1ccc(-c2ccc(NC(=O)C3N(C(=O)Nc4ccc5c(n4)OC(F)(F)O5)CC3(C)C)cc2)c(C)n1 ; Cc1nc(C(=O)O)ccc1-c1ccc(NC(=O)C2N(C(=O)Nc3ccc4c(n3)OC(F)(F)O4)CC2(C)C)cc1]

A mixture of methyl 5-(4-(1-((2,2-difluoro-[1,3]dioxolo[4,5-b]pyridin-5-yl)carbamoyl)-3,3-dimethylazetidine-2-carboxamido)phenyl)-6-methylpicolinate (70.0 mg, 0.126 mmol) and lithium hydroxide monohydrate (42.0 mg, 1.00 mmol) in tetrahydrofuran (1.5 mL) and water (1.5 mL) was stirred at room temperature for 1 hour. Upon completion, the reaction mixture was directly purified by reversed-phase chromatography (conditions: column: C18 silica gel; mobile phase A: water (0.1% formic acid); mobile phase B: acetonitrile; gradient (B %): 0% to 100% over 20 min; detector: UV 254 nm) to afford 5-(4-(1-((2,2-difluoro-[1,3]dioxolo[4,5-b]pyridin-5-yl)carbamoyl)-3,3-dimethylazetidine-2-carboxamido)phenyl)-6-methylpicolinic acid (42 mg, 61%) as a tan semi-solid. LCMS (ESI) [M+H]⁺: 540.5.

### Step 4: (S)-5-(4-(1-((2,2-difluoro-[1,3]dioxolo[4,5-b]pyridin-5-yl)carbamoyl)-3,3-dimethylazetidine-2-carboxamido)phenyl)-6-methylpicolinic acid and (R)-5-(4-(1-((2,2-difluoro-[1,3]dioxolo[4,5-b]pyridin-5-yl)carbamoyl)-3,3-dimethylazetidine-2-carboxamido)phenyl)-6-methylpicolinic acid

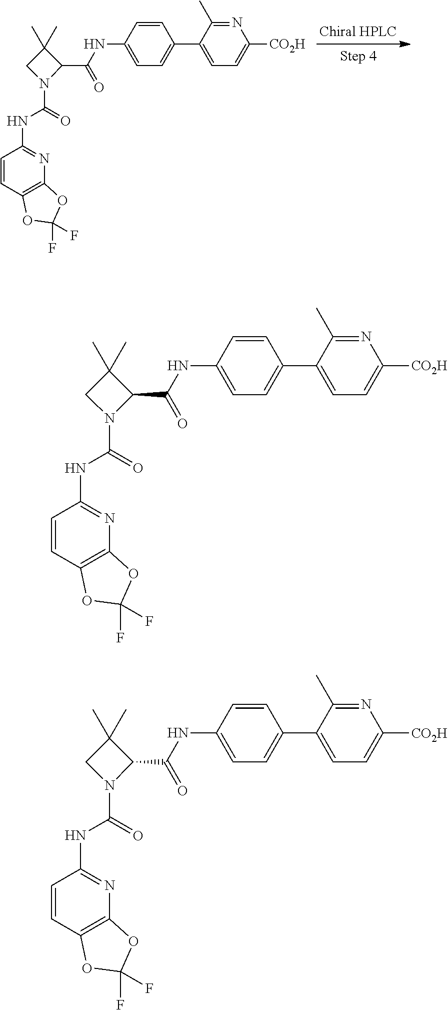 [SMILES: Cc1nc(C(=O)O)ccc1-c1ccc(NC(=O)C2N(C(=O)Nc3ccc4c(n3)OC(F)(F)O4)CC2(C)C)cc1 ; Cc1nc(C(=O)O)ccc1-c1ccc(NC(=O)[C@H]2N(C(=O)Nc3ccc4c(n3)OC(F)(F)O4)CC2(C)C)cc1 ; Cc1nc(C(=O)O)ccc1-c1ccc(NC(=O)[C@@H]2N(C(=O)Nc3ccc4c(n3)OC(F)(F)O4)CC2(C)C)cc1]

5-(4-(1-((2,2-difluoro-[1,3]dioxolo[4,5-b]pyridin-5-yl)carbamoyl)-3,3-dimethylazetidine-2-carboxamido)phenyl)-6-methylpicolinic acid (40 mg) was resolved by preparative chiral HPLC (conditions: column: CHIRAL ART Cellulose-SB, 3*25 cm, 5 μm; mobile phase A: hexane (0.1% formic acid), mobile phase B: ethanol; flow rate: 40 mL/min; Gradient (B %): isocratic 50% B; detector: 220/254 nm; RT1 (min): 8.5; RT2 (min): 18.5) to afford (Compound 189a; Method AY, Peak 1, 1.50 min) (S)-5-(4-(1-((2,2-difluoro-[1,3]dioxolo[4,5-b]pyridin-5-yl)carbamoyl)-3,3-dimethylazetidine-2-carboxamido)phenyl)-6-methylpicolinic acid (1^{st} peak, 6.5 mg, 16%) as a white solid and (Compound 189b; Method AY, Peak 2, 3.20 min) (R)-5-(4-(1-((2,2-difluoro-[1,3]dioxolo[4,5-b]pyridin-5-yl)carbamoyl)-3,3-dimethylazetidine-2-carboxamido)phenyl)-6-methylpicolinic acid (2^{nd} peak, 6.3 mg, 16%) as a white solid.

| Cmpd # | Characterization Data |
|---|---|
| 189a | LCMS (ESI) [M + H]⁺: 540.3 |
| | ¹H NMR (400 MHz, DMSO-d₆) δ 10.08 (s, 1H), 9.51 (s, 1H), 7.91 (d, J = 7.9 Hz, 1H), 7.77 (dd, J = |
| | 17.4, 9.0 Hz, 5H), 7.41 (d, J = 8.2 Hz, 2H), 4.50 (s, 1H), 3.79 (d, J = 7.7 Hz, 1H), 3.66 (d, J = 7.8 Hz, |
| | 1H), 2.45 (s, 3H), 1.42 (s, 3H), 1.11 (s, 3H). |
| 189b | LCMS (ESI) [M + H]⁺: 540.2 |
| | ¹H NMR (400 MHz, DMSO-d₆) δ 10.01 (s, 1H), 9.43 (s, 1H), 7.84 (d, J = 7.9 Hz, 1H), 7.78-7.62 |
| | (m, 5H), 7.31 (d, J = 8.1 Hz, 2H), 4.43 (s, 1H), 3.72 (d, J = 7.7 Hz, 1H), 3.59 (d, J = 7.8 Hz, 1H), |
| | 2.41 (s, 3H), 1.35 (s, 3H), 1.04 (s, 3H). |

### Example 190: (S)-2-(5-(1H-benzo[d][1,2,3]triazol-5-yl)-3-fluoropyridin-2-yl)-N-(2-cyclopropylpyrimidin-5-yl)pyrrolidine-1-carboxamide and (R)-2-(5-(1H-benzo[d][1,2,3]triazol-5-yl)-3-fluoropyridin-2-yl)-N-(2-cyclopropylpyrimidin-5-yl)pyrrolidine-1-carboxamide

### Step 1: 2-(5-chloro-3-fluoropyridin-2-yl)-N-(2-cyclopropylpyrimidin-5-yl)pyrrolidine-1-carboxamide

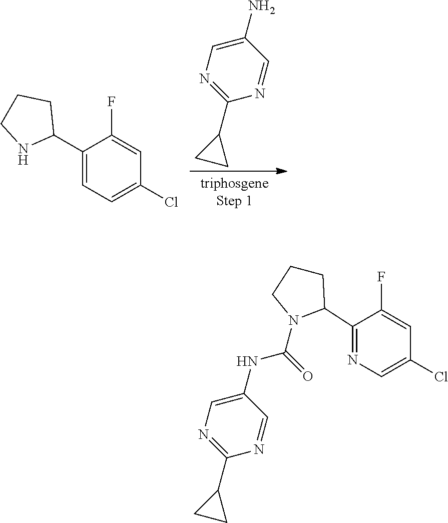 [SMILES: Fc1cc(Cl)ccc1C1CCCN1 ; Nc1cnc(C2CC2)nc1 ; O=C(Nc1cnc(C2CC2)nc1)N1CCCC1c1ncc(Cl)cc1F]

To a stirred solution of 2-cyclopropylpyrimidin-5-amine (243 mg, 1.79 mmol) and N, N-diisopropylethylamine (966 mg, 7.48 mmol) in dichloromethane (5.0 mL) at 0° C. was added triphosgene (177 mg, 0.598 mmol). After 10 minutes, 5-chloro-3-fluoro-2-(pyrrolidin-2-yl)pyridine (300 mg, 1.50 mmol) was added and the reaction was allowed to stir at 0° C. for an additional 1 hour. Upon completion, the reaction mixture was concentrated under reduced pressure. The crude residue was directly purified by silica gel column chromatography, eluted with petroleum ether/ethyl acetate (1:1) to afford 2-(5-chloro-3-fluoropyridin-2-yl)-N-(2-cyclopropylpyrimidin-5-yl)pyrrolidine-1-carboxamide (120 mg, 22%) as a yellow solid. LCMS (ESI) [M+H]⁺: 362.1.

### Step 2: 2-(5-(1H-benzo[d][1,2,3]triazol-5-yl)-3-fluoropyridin-2-yl)-N-(2-cyclopropylpyrimidin-5-yl)pyrrolidine-1-carboxamide

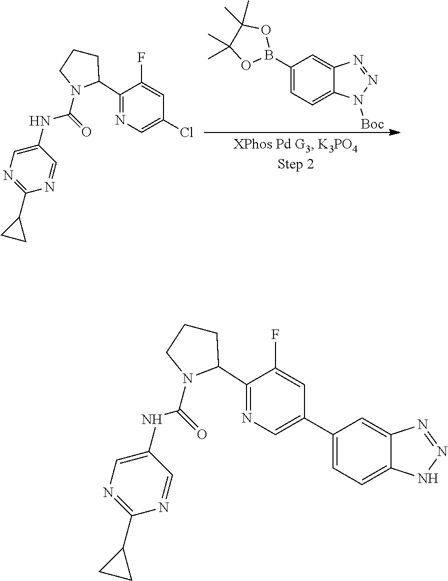 [SMILES: O=C(Nc1cnc(C2CC2)nc1)N1CCCC1c1ncc(Cl)cc1F ; CC(C)(C)OC(=O)n1nnc2cc(B3OC(C)(C)C(C)(C)O3)ccc21 ; O=C(Nc1cnc(C2CC2)nc1)N1CCCC1c1ncc(-c2ccc3[nH]nnc3c2)cc1F]

A solution of 2-(5-chloro-3-fluoropyridin-2-yl)-N-(2-cyclopropylpyrimidin-5-yl)pyrrolidine-1-carboxamide (100 mg, 0.276 mmol), tert-butyl 5-(4,4,5,5-tetramethyl-1,3,2-dioxaborolan-2-yl)-1,2,3-benzotriazole-1-carboxylate (114 mg, 0.331 mmol), XPhos Pd G3 (46.8 mg, 0.055 mmol) and tribasic potassium phosphate (176 mg, 0.828 mmol) in tetrahydrofuran (1.0 mL) and water (0.10 mL) was stirred at 90° C. for 1 hour. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 20 min; detector: UV 254 nm) to afford 2-(5-(1H-benzo[d][1,2,3]triazol-5-yl)-3-fluoropyridin-2-yl)-N-(2-cyclopropylpyrimidin-5-yl)pyrrolidine-1-carboxamide (25 mg, 20%) as a white solid. LCMS (ESI) [M+H]⁺: 445.1.

### Step 3: (S)-2-(5-(1H-benzo[d][1,2,3]triazol-5-yl)-3-fluoropyridin-2-yl)-N-(2-cyclopropylpyrimidin-5-yl)pyrrolidine-1-carboxamide and (R)-2-(5-(1H-benzo[d][1,2,3]triazol-5-yl)-3-fluoropyridin-2-yl)-N-(2-cyclopropylpyrimidin-5-yl)pyrrolidine-1-carboxamide

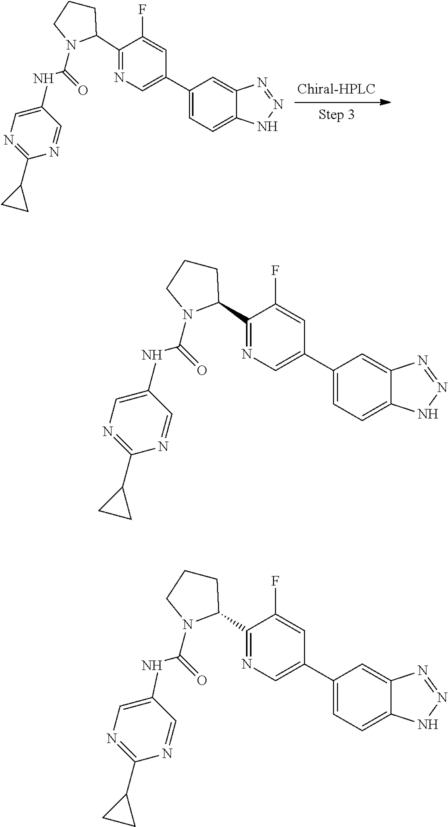 [SMILES: O=C(Nc1cnc(C2CC2)nc1)N1CCCC1c1ncc(-c2ccc3[nH]nnc3c2)cc1F ; O=C(Nc1cnc(C2CC2)nc1)N1CCC[C@H]1c1ncc(-c2ccc3[nH]nnc3c2)cc1F ; O=C(Nc1cnc(C2CC2)nc1)N1CCC[C@@H]1c1ncc(-c2ccc3[nH]nnc3c2)cc1F]

2-(5-(1H-benzo[d][1,2,3]triazol-5-yl)-3-fluoropyridin-2-yl)-N-(2-cyclopropylpyrimidin-5-yl)pyrrolidine-1-carboxamide (25 mg) was resolved by preparative chiral HPLC (conditions: column: Enantiocel-A4-5, 3.0*25 cm, 5 μm; mobile phase A: hexane (10 mM NH₃/methanol); mobile phase B: ethanol/acetonitrile (5:1); flow rate: 40 mL/min; gradient (B %): isocratic 30% B; detector: 220/254 nm; RT1 (min): 11.2; RT2 (min): 29.0) to afford (Compound 190a; Method AS, Peak 1, 1.27 min) (S)-2-(5-(1H-benzo[d][1,2,3]triazol-5-yl)-3-fluoropyridin-2-yl)-N-(2-cyclopropylpyrimidin-5-yl)pyrrolidine-1-carboxamide (1^{st} peak, 9.7 mg, 39%) as a white solid and (Compound 190b; Method AS, Peak 2, 3.61 min) (R)-2-(5-(1H-benzo[d][1,2,3]triazol-5-yl)-3-fluoropyridin-2-yl)-N-(2-cyclopropylpyrimidin-5-yl)pyrrolidine-1-carboxamide (2^{nd} peak, 7.6 mg, 30%) as a white solid.

| Cmpd # | Characterization Data |
|---|---|
| 190a | LCMS (ESI) [M + H]⁺: 445.2 |
| | ¹H NMR (400 MHz, methanol-d₄) δ 8.67 (s, 3H), 8.15 (s, 1H), 8.04-7.87 (m, 2H), 7.78 (dd, J = |
| | 8.7, 1.7 Hz, 1H), 5.54-5.44 (m, 1H), 3.95-3.83 (m, 1H), 3.79-3.66 (m, 1H), 2.55-2.42 (m, |
| | 1H), 2.24-2.03 (m, 4H), 1.00 (d, J = 6.5 Hz, 4H). |
| 190b | LCMS (ESI) [M + H]⁺: 445.2 |
| | ¹H NMR (400 MHz, methanol-d₄) δ 8.67 (s, 3H), 8.16 (s, 1H), 8.05-7.89 (m, 2H), 7.78 (dd, J = |
| | 8.6, 1.6 Hz, 1H), 5.51-5.43 (m, 1H), 3.94-3.83 (m, 1H), 3.78-3.67 (m, 1H), 2.53-2.41 (m, |
| | 1H), 2.24-2.01 (m, 4H), 1.00 (d, J = 6.5 Hz, 4H). |

### Example 191: (R)-2-(3-fluoro-5-(1H-indazol-5-yl)pyridin-2-yl)-N-(4-isopropylphenyl)pyrrolidine-1-carboxamide

### Step 1: tert-butyl (2R,4S)-2-(5-chloro-3-fluoropyridin-2-yl)-4-hydroxypyrrolidine-1-carboxylate

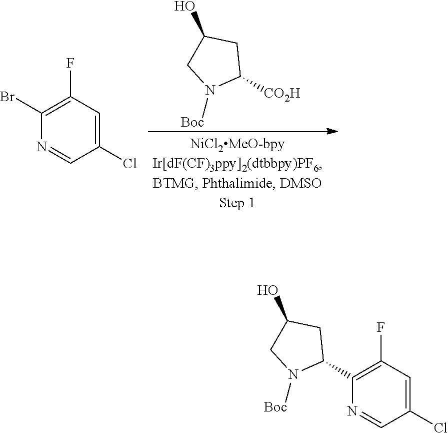 [SMILES: Fc1cc(Cl)cnc1Br ; CC(C)(C)OC(=O)N1C[C@@H](O)C[C@@H]1C(=O)O ; CC(C)(C)OC(=O)N1C[C@@H](O)C[C@@H]1c1ncc(Cl)cc1F]

A solution of dichloronickel·4,4′-dimethoxy-2,2′-bipyridine (1.49 g, 4.32 mmol), phthalimide (6.36 g, 43.2 mmol), Ir[dF(CF₃)ppy]₂(dtbbpy))PF₆ (0.97 g, 0.865 mmol), (2R,4S)-1-(tert-butoxycarbonyl)-4-hydroxypyrrolidine-2-carboxylic acid (10.0 g, 43.2 mmol), 2-bromo-5-chloro-3-fluoropyridine (9.10 g, 43.2 mmol), and N″-tert-butyl-N,N,N′,N′-tetramethylguanidine (11.1 g, 64.9 mmol) in DMSO (10.0 mL) at room temperature was stirred under 450 nm blue LED irradiation for 24 hours. Upon completion, the mixture was poured into water (50 mL) and extracted with ethyl acetate (10×10 mL). The combined layers were concentrated under reduced pressure. The crude residue was purified by silica gel column chromatography, eluted with petroleum ether/ethyl acetate (5:1) to afford tert-butyl (2R,4S)-2-(5-chloro-3-fluoropyridin-2-yl)-4-hydroxypyrrolidine-1-carboxylate (3.3 g, 24%) as a yellow oil. LCMS (ESI) [M+H]⁺: 317.1.

### Step 2: tert-butyl (2R,4S)-2-(5-chloro-3-fluoropyridin-2-yl)-4-((phenoxycarbonothioyl)oxy)pyrrolidine-1-carboxylate

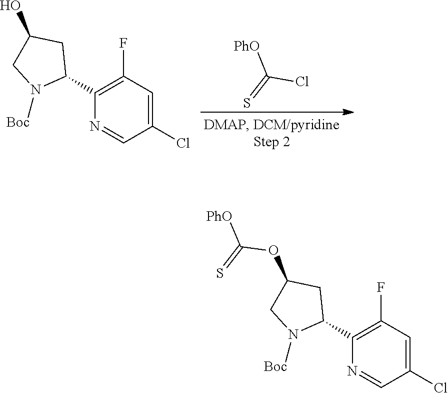 [SMILES: CC(C)(C)OC(=O)N1C[C@@H](O)C[C@@H]1c1ncc(Cl)cc1F ; S=C(Cl)Oc1ccccc1 ; CC(C)(C)OC(=O)N1C[C@@H](OC(=S)Oc2ccccc2)C[C@@H]1c1ncc(Cl)cc1F]

A solution of tert-butyl (2R,4S)-2-(5-chloro-3-fluoropyridin-2-yl)-4-hydroxypyrrolidine-1-carboxylate (3.30 g, 10.4 mmol), O-phenyl carbonochloridothioate (4.50 g, 26.0 mmol), pyridine (4.12 g, 52.1 mmol), and 4-dimethylaminopyridine (0.130 g, 1.04 mmol) in dichloromethane (20.0 mL) was stirred at room temperature for 1 hour. Upon completion, the reaction mixture was concentrated under reduced pressure to afford crude tert-butyl (2R,4S)-2-(5-chloro-3-fluoropyridin-2-yl)-4-((phenoxycarbonothioyl)oxy)pyrrolidine-1-carboxylate (6.00 g, crude) as a yellow oil, which was used directly in the next step without further purification. LCMS (ESI) [M+H]⁺: 453.1.

### Step 3: tert-butyl (R)-2-(5-chloro-3-fluoropyridin-2-yl)pyrrolidine-1-carboxylate

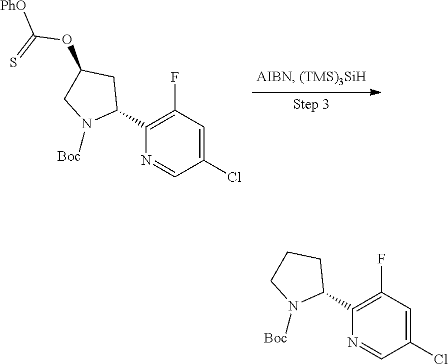 [SMILES: CC(C)(C)OC(=O)N1C[C@@H](OC(=S)Oc2ccccc2)C[C@@H]1c1ncc(Cl)cc1F ; CC(C)(C)OC(=O)N1CCC[C@@H]1c1ncc(Cl)cc1F]

A solution of tert-butyl (2R,4S)-2-(5-chloro-3-fluoropyridin-2-yl)-4-((phenoxycarbonothioyl)oxy)pyrrolidine-1-carboxylate (2.00 g, 4.41 mmol), AIBN (1.45 g, 8.83 mmol) and 1,1,1,3,3,3-hexamethyl-2-(trimethylsilyl)trisilane (2.20 g, 8.83 mmol) in toluene (10 mL) was stirred at 90° C. for 1 hour. Upon completion, the reaction mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford tert-butyl (R)-2-(5-chloro-3-fluoropyridin-2-yl)pyrrolidine-1-carboxylate (600 mg, 45%) as a colorless oil. LCMS (ESI) [M+H]⁺: 301.7.

### Step 4: (R)-5-chloro-3-fluoro-2-(pyrrolidin-2-yl)pyridine

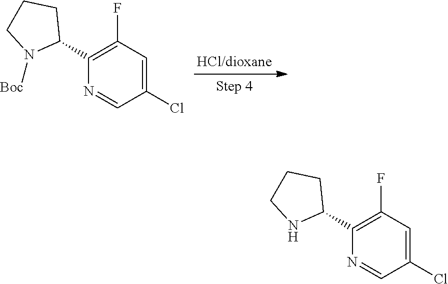 [SMILES: CC(C)(C)OC(=O)N1CCC[C@@H]1c1ncc(Cl)cc1F ; Fc1cc(Cl)cnc1[C@H]1CCCN1]

To a solution of tert-butyl (R)-2-(5-chloro-3-fluoropyridin-2-yl)pyrrolidine-1-carboxylate (2.00 g, 6.65 mmol) in 1,4-dioxane (5.0 mL) at room temperature was added a solution of hydrochloric acid (15 mL, 60 mmol, 4.0 M in 1,4-dioxane), and the resultant mixture was stirred for 1 hour. Upon completion, the mixture was concentrated under reduced pressure to afford crude (R)-5-chloro-3-fluoro-2-(pyrrolidin-2-yl)pyridine (1.5 g, crude) as a yellow solid, which was used directly in the next step without further purification. LCMS (ESI) [M+H]⁺: 201.6.

### Step 5: (R)-2-(5-chloro-3-fluoropyridin-2-yl)-N-(4-isopropylphenyl)pyrrolidine-1-carboxamide

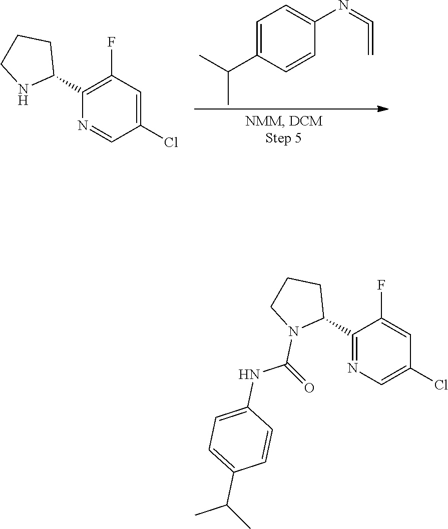 [SMILES: Fc1cc(Cl)cnc1[C@H]1CCCN1 ; C=C=Nc1ccc(C(C)C)cc1 ; CC(C)c1ccc(NC(=O)N2CCC[C@@H]2c2ncc(Cl)cc2F)cc1]

A solution of 1-isocyanato-4-isopropylbenzene (1.21 g, 7.48 mmol), 4-methylmorpholine (2.27 g, 22.4 mmol), and (R)-5-chloro-3-fluoro-2-(pyrrolidin-2-yl)pyridine (1.5 g, 7.48 mmol) in dichloromethane (8.0 mL) was stirred at room temperature for 1 hour. Upon completion, the mixture was concentrated under reduced pressure. The crude residue was directly purified by silica gel column chromatography, eluted with petroleum ether/ethyl acetate (1:1) to afford (R)-2-(5-chloro-3-fluoropyridin-2-yl)-N-(4-isopropylphenyl)pyrrolidine-1-carboxamide (1.60 g, 59%) as a colorless oil. LCMS (ESI) [M+H]⁺: 362.1.

### Step 6: (R)-2-(3-fluoro-5-(1H-indazol-5-yl)pyridin-2-yl)-N-(4-isopropylphenyl)pyrrolidine-1-carboxamide

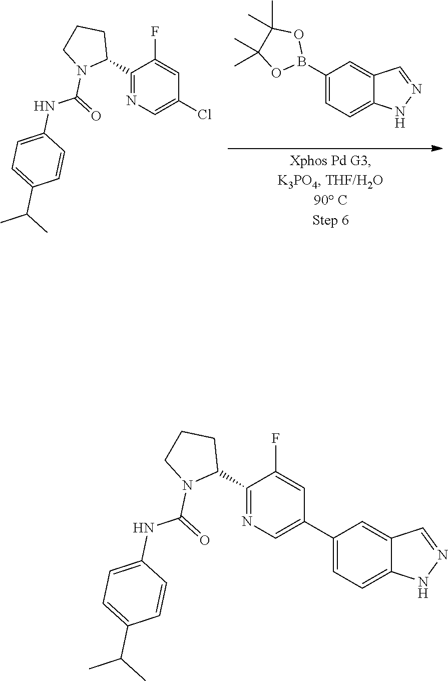

A solution of (R)-2-(5-chloro-3-fluoropyridin-2-yl)-N-(4-isopropylphenyl)pyrrolidine-1-carboxamide (60.0 mg, 0.166 mmol), 5-(4,4,5,5-tetramethyl-1,3,2-dioxaborolan-2-yl)-1H-indazole (60.7 mg, 0.249 mmol), tribasic potassium phosphate (106 mg, 0.498 mmol), and XPhos Pd G3 (28.0 mg, 0.033 mmol) in tetrahydrofuran (2.0 mL) and water (1.0 mL) was stirred at 90° C. for 1 hour. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford (Compound 191) (R)-2-(3-fluoro-5-(1H-indazol-5-yl)pyridin-2-yl)-N-(4-isopropylphenyl)pyrrolidine-1-carboxamide (28.3 mg, 38%) as a white solid.

| Cmpd # | Characterization Data |
|---|---|
| 191 | LCMS (ESI) [M + H]⁺: 444.2 |
| | ¹H NMR (400 MHz, DMSO-d₆) δ 13.20 (s, 1H), 8.70 (s, 1H), 8.14 (s, 3H), 7.99 (dd, J = 11.7, 1.9 Hz, |
| | 1H), 7.73 (d, J = 9.2 Hz, 1H), 7.64 (d, J = 8.7 Hz, 1H), 7.34 (d, J = 8.4 Hz, 2H), 7.04 (d, J = 8.4 Hz, |
| | 2H), 5.42-5.25 (m, 1H), 3.83-3.71 (m, 1H), 3.71-3.55 (m, 1H), 2.86-2.70 (m, 1H), 2.43-2.27 |
| | (m, 1H), 2.21-2.08 (m, 1H), 2.08-1.96 (m, 1H), 1.96-1.85 (m, 1H), 1.14 (d, J = 6.9 Hz, 6H). |

### Example 192: 4-(5-fluoro-6-((2R,4S)-4-methyl-1-((4-methyl-5-(1-(trifluoromethyl)cyclopropyl)pyridin-2-yl)carbamoyl)pyrrolidin-2-yl)pyridin-3-yl)benzoic acid and 4-(5-fluoro-6-((2R,4R)-4-methyl-1-((4-methyl-5-(1-(trifluoromethyl)cyclopropyl)pyridin-2-yl)carbamoyl)pyrrolidin-2-yl)pyridin-3-yl)benzoic acid

### Step 1: methyl 4-(5-fluoro-6-((2R)-4-methyl-1-((4-methyl-5-(1-(trifluoromethyl)cyclopropyl)pyridin-2-yl)carbamoyl)pyrrolidin-2-yl)pyridin-3-yl)benzoate

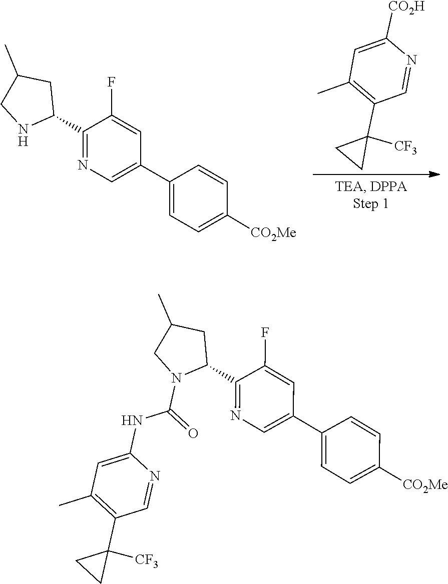 [SMILES: COC(=O)c1ccc(-c2cnc([C@H]3CC(C)CN3)c(F)c2)cc1 ; Cc1cc(C(=O)O)ncc1C1(C(F)(F)F)CC1 ; COC(=O)c1ccc(-c2cnc([C@H]3CC(C)CN3C(=O)Nc3cc(C)c(C4(C(F)(F)F)CC4)cn3)c(F)c2)cc1]

A solution of methyl 4-(5-fluoro-6-((2R)-4-methylpyrrolidin-2-yl)pyridin-3-yl)benzoate (100 mg, 0.310 mmol), 4-methyl-5-(1-(trifluoromethyl)cyclopropyl)picolinic acid (78.0 mg, 0.31 mmol), DPPA (262 mg, 0.95 mmol) and triethylamine (96 mg, 0.95 mmol) in toluene (2.0 mL) was stirred at 80° C. for 2 hours. Upon completion, the reaction mixture was concentrated under reduced pressure. The crude residue was directly purified by silica gel column chromatography, eluted with petroleum ether/ethyl acetate (1:1) to afford methyl 4-(5-fluoro-6-((2R)-4-methyl-1-((4-methyl-5-(1-(trifluoromethyl)cyclopropyl)pyridin-2-yl)carbamoyl)pyrrolidin-2-yl)pyridin-3-yl)benzoate (60.0 mg, 34%) as a white solid. LCMS (ESI) [M+H]⁺: 557.2.

### Step 2: 4-(5-fluoro-6-((2R)-4-methyl-1-((4-methyl-5-(1-(trifluoromethyl)cyclopropyl)pyridin-2-yl)carbamoyl)pyrrolidin-2-yl)pyridin-3-yl)benzoic acid

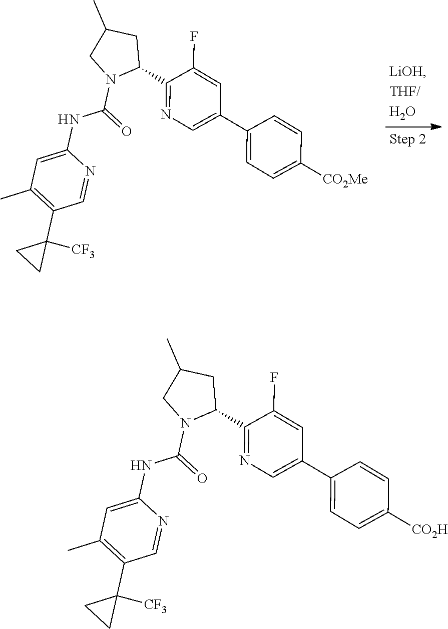 [SMILES: COC(=O)c1ccc(-c2cnc([C@H]3CC(C)CN3C(=O)Nc3cc(C)c(C4(C(F)(F)F)CC4)cn3)c(F)c2)cc1 ; Cc1cc(NC(=O)N2CC(C)C[C@@H]2c2ncc(-c3ccc(C(=O)O)cc3)cc2F)ncc1C1(C(F)(F)F)CC1]

A solution of methyl 4-(5-fluoro-6-((2R)-4-methyl-1-((4-methyl-5-(1-(trifluoromethyl)cyclopropyl)pyridin-2-yl)carbamoyl)pyrrolidin-2-yl)pyridin-3-yl)benzoate (60 mg, 0.100 mmol) and lithium hydroxide monohydrate (45.4 mg, 1.08 mmol) in tetrahydrofuran (2.0 mL) and water (0.40 mL) was stirred at room temperature for 30 minutes. Upon completion, the reaction mixture was directly purified by reversed-phase flash chromatography (conditions: column: C18 silica gel; mobile phase A: water (0.1% formic acid); mobile phase B: acetonitrile; gradient (B %): 0% to 100% over 20 min; detector: UV 254 nm) to afford 4-(5-fluoro-6-((2R)-4-methyl-1-((4-methyl-5-(1-(trifluoromethyl)cyclopropyl)pyridin-2-yl)carbamoyl)pyrrolidin-2-yl)pyridin-3-yl)benzoic acid (46.0 mg, 78%) as a white solid. LCMS (ESI) [M+H]⁺: 553.1.

### Step 3: 4-(5-fluoro-6-((2R,4S)-4-methyl-1-((4-methyl-5-(1-(trifluoromethyl)cyclopropyl)pyridin-2-yl)carbamoyl)pyrrolidin-2-yl)pyridin-3-yl)benzoic acid and 4-(5-fluoro-6-((2R,4R)-4-methyl-1-((4-methyl-5-(1-(trifluoromethyl)cyclopropyl)pyridin-2-yl)carbamoyl)pyrrolidin-2-yl)pyridin-3-yl)benzoic acid

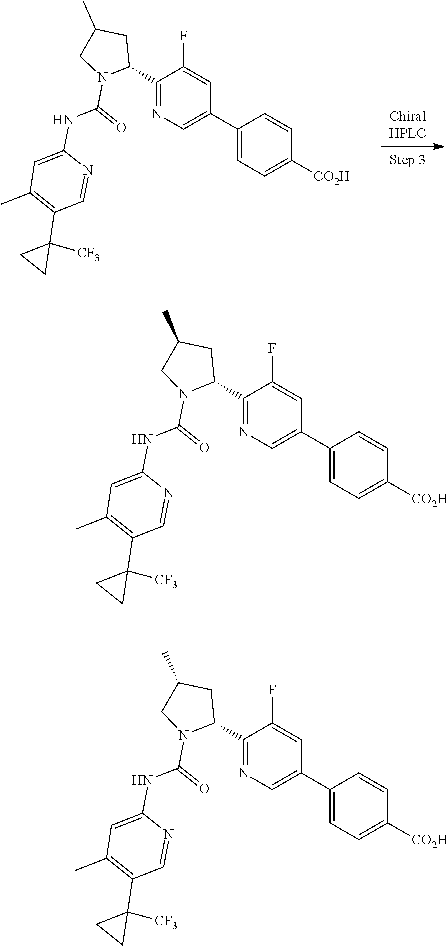 [SMILES: Cc1cc(NC(=O)N2CC(C)C[C@@H]2c2ncc(-c3ccc(C(=O)O)cc3)cc2F)ncc1C1(C(F)(F)F)CC1 ; Cc1cc(NC(=O)N2C[C@@H](C)C[C@@H]2c2ncc(-c3ccc(C(=O)O)cc3)cc2F)ncc1C1(C(F)(F)F)CC1 ; Cc1cc(NC(=O)N2C[C@H](C)C[C@@H]2c2ncc(-c3ccc(C(=O)O)cc3)cc2F)ncc1C1(C(F)(F)F)CC1]

4-(5-fluoro-6-((2R)-4-methyl-1-((4-methyl-5-(1-(trifluoromethyl)cyclopropyl)pyridin-2-yl)carbamoyl)pyrrolidin-2-yl)pyridin-3-yl)benzoic acid (46.0 mg) was resolved by preparative chiral HPLC (conditions: column: CHIRALPAK ID, 3*25 cm, 5 μm; mobile phase A: hexane/MTBE (1:1, 0.1% formic acid); mobile phase B: ethanol; flow rate: 40 mL/min; gradient (B %): isocratic 10% B; detector: 220/254 nm; RT1 (min): 21.5; RT2 (min): 28.3) to afford (Compound 192a; Method AU, Peak 1, 1.91 min) 4-(5-fluoro-6-((2R,4S)-4-methyl-1-((4-methyl-5-(1-(trifluoromethyl)cyclopropyl)pyridin-2-yl)carbamoyl)pyrrolidin-2-yl)pyridin-3-yl)benzoic acid (1^{st} peak, 20.7 mg, 46%) as a white solid, and (Compound 192b; Method AU, Peak 2, 2.55 min) 4-(5-fluoro-6-((2R,4R)-4-methyl-1-((4-methyl-5-(1-(trifluoromethyl)cyclopropyl)pyridin-2-yl)carbamoyl)pyrrolidin-2-yl)pyridin-3-yl)benzoic acid (2^{nd} peak, 5.9 mg, 15%) as a white solid.

| Cmpd # | Characterization Data |
|---|---|
| 192a | LCMS (ESI) [M + H]⁺: 543.2 |
| | ¹H NMR (400 MHz, methanol-d₄) δ 8.67-8.61 (m, 1H), 8.20-8.05 (m, 3H), 7.95-7.86 (m, 1H), |
| | 7.83-7.73 (m, 2H), 7.68 (s, 1H), 5.57-5.46 (m, 1H), 4.02 (dd, J = 9.2, 7.5 Hz, 1H), 3.28-3.21 |
| | (m, 1H), 2.76-2.58 (m, 1H), 2.44-2.35 (m, 3H), 2.17-2.06 (m, 2H), 1.49-1.40 (m, 2H), 1.16 |
| | (dd, J = 6.6, 1.4 Hz, 3H), 1.06 (s, 2H). |
| 192b | LCMS (ESI) [M + H]⁺: 543.2 |
| | ¹H NMR (400 MHz, methanol-d₄) δ 8.65 (d, J = 1.6 Hz, 1H), 8.18-8.08 (m, 3H), 7.90 (dd, J = |
| | 11.1, 1.9 Hz, 1H), 7.78 (dd, J = 8.2, 2.3 Hz, 2H), 7.59 (s, 1H), 5.39 (dd, J = 9.7, 7.0 Hz, 1H), 3.97 (t, |
| | J = 8.4 Hz, 1H), 3.37 (t, J = 9.7 Hz, 1H), 2.69-2.47 (m, 2H), 2.44-2.34 (m, 3H), 1.73 (q, J = 11.1 |
| | Hz, 1H), 1.46-1.38 (m, 2H), 1.19 (d, J = 6.3 Hz, 3H), 1.05 (s, 2H). |

### Example 193: (R)-2-(3-fluoro-5-(1-methyl-1H-benzo[d][1,2,3]triazol-6-yl)pyridin-2-yl)-N-(4-isopropylphenyl)pyrrolidine-1-carboxamide

### Step 1: (R)-2-(3-fluoro-5-(1-methyl-1H-benzo[d][1,2,3]triazol-6-yl)pyridin-2-yl)-N-(4-isopropylphenyl)pyrrolidine-1-carboxamide

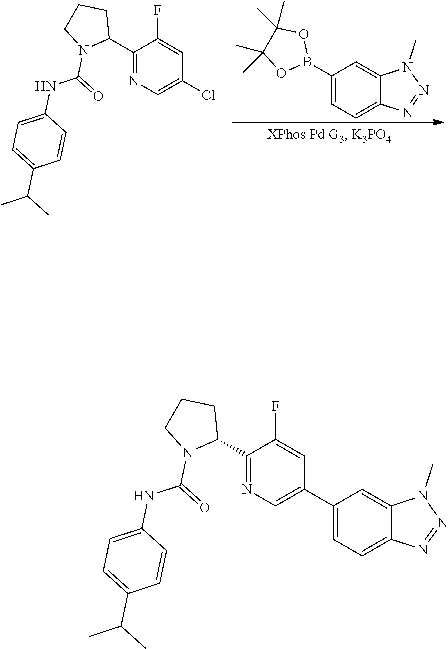 [SMILES: CC(C)c1ccc(NC(=O)N2CCCC2c2ncc(Cl)cc2F)cc1 ; Cn1nnc2ccc(B3OC(C)(C)C(C)(C)O3)cc21 ; CC(C)c1ccc(NC(=O)N2CCC[C@@H]2c2ncc(-c3ccc4nnn(C)c4c3)cc2F)cc1]

A solution of (R)-2-(5-chloro-3-fluoropyridin-2-yl)-N-(4-isopropylphenyl)pyrrolidine-1-carboxamide (84.0 mg, 0.232 mmol), 1-methyl-6-(4,4,5,5-tetramethyl-1,3,2-dioxaborolan-2-yl)-1H-benzo[d][1,2,3]triazole (60 mg, 0.232 mmol), tribasic potassium phosphate (147 mg, 0.696 mmol), and XPhos Pd G3 (19.6 mg, 0.023 mmol) in tetrahydrofuran (2.0 mL) and water (1.0 mL) was stirred at 90° C. under nitrogen atmosphere for 1 hour. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford (Compound 193) (R)-2-(3-fluoro-5-(1-methyl-1H-benzo[d][1,2,3]triazol-6-yl)pyridin-2-yl)-N-(4-isopropylphenyl)pyrrolidine-1-carboxamide (22.0 mg, 21%) as a white solid.

| Cmpd # | Characterization Data |
|---|---|
| 193 | LCMS (ESI) [M + H]⁺: 459.2 |
| | ¹H NMR (400 MHz, Methanol-d₄) δ 8.72 (s, 1H), 8.13-8.05 (m, 2H), 8.01 (dd, J = 11.2, 2.0 Hz, |
| | 1H), 7.78-7.70 (m, 1H), 7.25 (d, J = 8.5 Hz, 2H), 7.09 (d, J = 8.5 Hz, 2H), 5.53-5.41 (m, 1H), |
| | 4.39 (s, 3H), 3.92-3.82 (m, 1H), 3.77-3.65 (m, 1H), 2.89-2.76 (m, 1H), 2.55-2.40 (m, 1H), |
| | 2.25-2.00 (m, 3H), 1.19 (d, J = 6.9 Hz, 6H). |

### Example 194: (R)-2-(3-fluoro-5-(1-methyl-1H-benzo[d][1,2,3]triazol-5-yl)pyridin-2-yl)-N-(4-isopropylphenyl)pyrrolidine-1-carboxamide

### Step 1: (R)-2-(3-fluoro-5-(1-methyl-1H-benzo[d][1,2,3]triazol-5-yl)pyridin-2-yl)-N-(4-isopropylphenyl)pyrrolidine-1-carboxamide

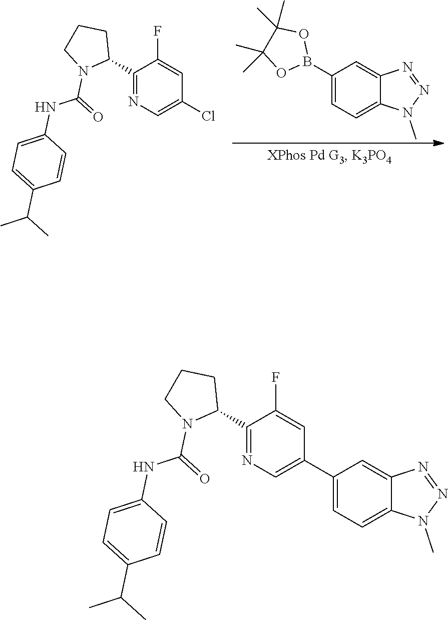 [SMILES: CC(C)c1ccc(NC(=O)N2CCC[C@@H]2c2ncc(Cl)cc2F)cc1 ; Cn1nnc2cc(B3OC(C)(C)C(C)(C)O3)ccc21 ; CC(C)c1ccc(NC(=O)N2CCC[C@@H]2c2ncc(-c3ccc4c(c3)nnn4C)cc2F)cc1]

A solution of (R)-2-(5-chloro-3-fluoropyridin-2-yl)-N-(4-isopropylphenyl)pyrrolidine-1-carboxamide (84.0 mg, 0.232 mmol), 1-methyl-5-(4,4,5,5-tetramethyl-1,3,2-dioxaborolan-2-yl)-1H-benzo[d][1,2,3]triazole (60.0 mg, 0.232 mmol), tribasic potassium phosphate (147 mg, 0.696 mmol) and XPhos Pd G3 (19.6 mg, 0.023 mmol) in tetrahydrofuran (2.0 mL) and water (1.0 mL) was stirred at 90° C. for 1 hour. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford (Compound 194) (R)-2-(3-fluoro-5-(1-methyl-1H-benzo[d][1,2,3]triazol-5-yl)pyridin-2-yl)-N-(4-isopropylphenyl)pyrrolidine-1-carboxamide (19.4 mg 18%) as a white solid.

| Cmpd # | Characterization Data |
|---|---|
| 194 | LCMS (ESI) [M + H]⁺: 459.3 |
| | ¹H NMR (400 MHz, methanol-d₄) δ 8.67 (s, 1H), 8.27 (s, 1H), 7.93 (dd, J = 11.2, 1.9 Hz, 1H), 7.88 |
| | (s, 2H), 7.25 (d, J = 8.4 Hz, 2H), 7.09 (d, J = 8.3 Hz, 2H), 5.53-5.42 (m, 1H), 4.37 (s, 3H), 3.95- |
| | 3.81 (m, 1H), 3.76-3.64 (m, 1H), 2.89-2.75 (m, 1H), 2.52-2.41 (m, 1H), 2.26-1.99 (m, 3H), |
| | 1.19 (d, J = 6.9 Hz, 6H). |

### Example 195: (R)-2-(5-(1H-benzo[d]imidazol-5-yl)-3-fluoropyridin-2-yl)-N-(4-isopropylphenyl)pyrrolidine-1-carboxamide

### Step 1: (R)-2-(5-(1H-benzo[d]imidazol-5-yl)-3-fluoropyridin-2-yl)-N-(4-isopropylphenyl)pyrrolidine-1-carboxamide

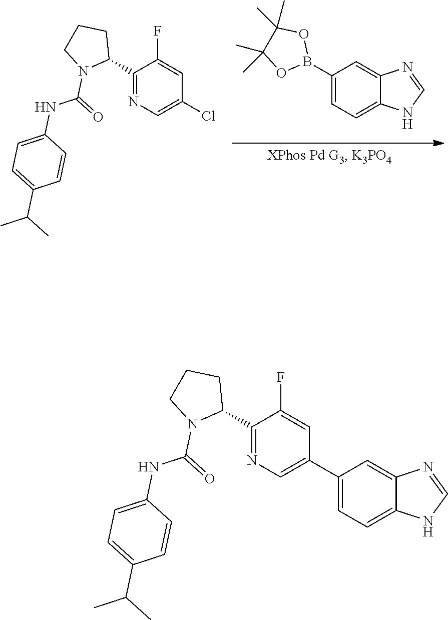 [SMILES: CC(C)c1ccc(NC(=O)N2CCC[C@@H]2c2ncc(Cl)cc2F)cc1 ; CC1(C)OB(c2ccc3[nH]cnc3c2)OC1(C)C ; CC(C)c1ccc(NC(=O)N2CCC[C@@H]2c2ncc(-c3ccc4[nH]cnc4c3)cc2F)cc1]

A solution of (R)-2-(5-chloro-3-fluoropyridin-2-yl)-N-(4-isopropylphenyl)pyrrolidine-1-carboxamide (89.0 mg, 0.246 mmol), 5-(4,4,5,5-tetramethyl-1,3,2-dioxaborolan-2-yl)-1H-benzo[d]imidazole (60 mg, 0.246 mmol), tribasic potassium phosphate (156 mg, 0.738 mmol) and XPhos Pd G3 (20.8 mg, 0.025 mmol) in tetrahydrofuran (2.0 mL) and water (1.0 mL) was stirred at 90° C. for 1 hour. Upon completion, the mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford (Compound 195) (R)-2-(5-(1H-benzo[d]imidazol-5-yl)-3-fluoropyridin-2-yl)-N-(4-isopropylphenyl)pyrrolidine-1-carboxamide (8.1 mg, 7%) as a white solid.

| Cmpd # | Characterization Data |
|---|---|
| 195 | LCMS (ESI) [M + H]⁺: 444.2 |
| | ¹H NMR (400 MHz, methanol-d₄) δ 8.62 (s, 1H), 8.24 (s, 1H), 7.89 (d, J = 1.7 Hz, 1H), 7.85 (dd, |
| | J = 11.4, 1.9 Hz, 1H), 7.72 (d, J = 8.4 Hz, 1H), 7.58 (dd, J = 8.5, 1.7 Hz, 1H), 7.25 (d, J = 8.5 Hz, |
| | 2H), 7.09 (d, J = 8.5 Hz, 2H), 5.49-5.41 (m, 1H), 3.92-3.83 (m, 1H), 3.74-3.65 (m, 1H), |
| | 2.89-2.76 (m, 1H), 2.51-2.40 (m, 1H), 2.26-1.97 (m, 3H), 1.19 (d, J = 6.9 Hz, 6H). |

### Example 196: (S)-5-(4-(1-((2-difluoro-[1,3]dioxolo[4,5-b]pyridin-6-yl)carbamoyl)-3,3-dimethylazetidine-2-carboxamido)phenyl)-6-methylpicolinic acid and (R)-5-(4-(1-((2,2-difluoro-[1,3]dioxolo[4,5-b]pyridin-6-yl)carbamoyl)-3,3-dimethylazetidine-2-carboxamido)phenyl)-6-methylpicolinic acid

### Step 1: 1-(tert-butoxycarbonyl)-3,3-dimethylazetidine-2-carboxylic acid

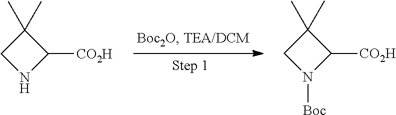

To a mixture of 3,3-dimethylazetidine-2-carboxylic acid (200 mg, 1.54 mmol) in dichloromethane (4.0 mL) at 0° C. were added triethylamine (313 mg, 3.09 mmol) and di-tert-butyldicarbonate (506 mg, 2.32 mmol). The resultant mixture was warmed to room temperature and stirred for 16 hours. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 25 min; detector: UV 254/220 nm) to afford 1-(tert-butoxycarbonyl)-3,3-dimethylazetidine-2-carboxylic acid (120 mg, 33% yield) as a white solid. LCMS (ESI) [M+H]⁺: 230.1.

### Step 2: methyl 5-(4-(1-(tert-butoxycarbonyl)-3,3-dimethylazetidine-2-carboxamido)phenyl)-6-methylpicolinate

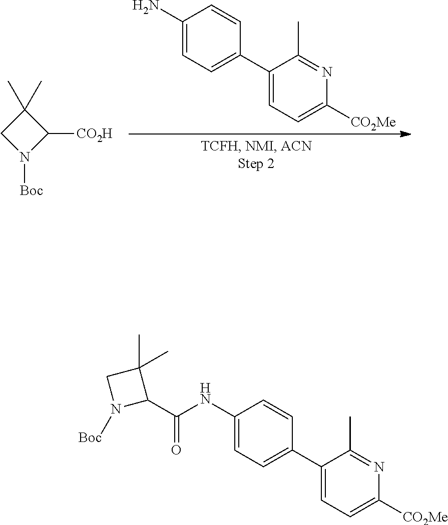 [SMILES: CC(C)(C)OC(=O)N1CC(C)(C)C1C(=O)O ; COC(=O)c1ccc(-c2ccc(N)cc2)c(C)n1 ; COC(=O)c1ccc(-c2ccc(NC(=O)C3N(C(=O)OC(C)(C)C)CC3(C)C)cc2)c(C)n1]

To a mixture of 1-(tert-butoxycarbonyl)-3,3-dimethylazetidine-2-carboxylic acid (100 mg, 0.436 mmol) and methyl 5-(4-aminophenyl)-6-methylpicolinate (105 mg, 0.436 mmol) in acetonitrile (6.0 mL) were added 1-methyl-1H-imidazole (107 mg, 1.30 mmol) and TCFH (183 mg, 0.654 mmol) at room temperature. After 2 hours, the reaction mixture was concentrated under reduced pressure. The crude residue was directly purified by silica gel column chromatography, eluted with petroleum ether/ethyl acetate (1:1) to afford methyl 5-(4-(1-(tert-butoxycarbonyl)-3,3-dimethylazetidine-2-carboxamido)phenyl)-6-methylpicolinate (150 mg, 75%) as a white solid. LCMS (ESI) [M+H]⁺: 454.2.

### Step 3: methyl 5-(4-(3,3-dimethylazetidine-2-carboxamido)phenyl)-6-methylpicolinate

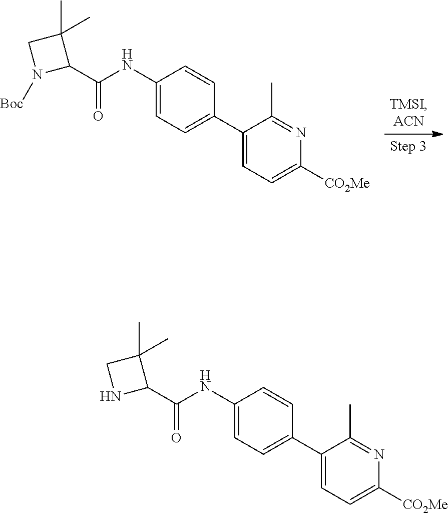 [SMILES: COC(=O)c1ccc(-c2ccc(NC(=O)C3N(C(=O)OC(C)(C)C)CC3(C)C)cc2)c(C)n1 ; COC(=O)c1ccc(-c2ccc(NC(=O)C3NCC3(C)C)cc2)c(C)n1]

A solution of methyl 5-(4-(1-(tert-butoxycarbonyl)-3,3-dimethylazetidine-2-carboxamido)phenyl)-6-methylpicolinate (150 mg, 0.330 mmol) in acetonitrile (5.0 mL) at 0° C. was treated with TMSI (198 mg, 0.990 mmol). After 5 minutes, the reaction mixture was warmed to room temperature and was allowed to stir for an additional 30 minutes. Upon completion, the reaction mixture was concentrated under reduced pressure to afford methyl 5-(4-(3,3-dimethylazetidine-2-carboxamido)phenyl)-6-methylpicolinate (120 mg, crude) as a dark yellow solid, which was used directly in the next step without further purification. LCMS (ESI) [M+H]⁺: 354.2.

### Step 4: methyl 5-(4-(1-((2,2-difluoro-[1,3]dioxolo[4,5-b]pyridin-6-yl)carbamoyl)-3,3-dimethylazetidine-2-carboxamido)phenyl)-6-methylpicolinate

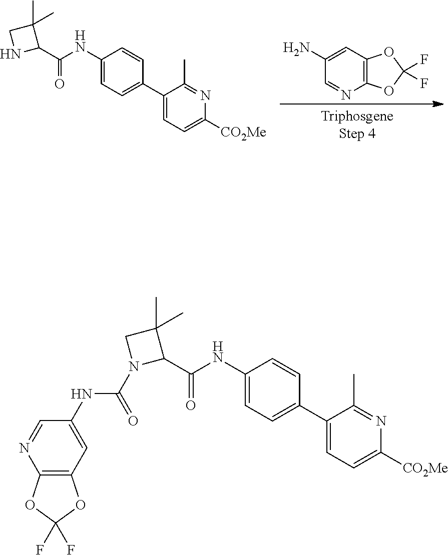 [SMILES: COC(=O)c1ccc(-c2ccc(NC(=O)C3NCC3(C)C)cc2)c(C)n1 ; Nc1cnc2c(c1)OC(F)(F)O2 ; COC(=O)c1ccc(-c2ccc(NC(=O)C3N(C(=O)Nc4cnc5c(c4)OC(F)(F)O5)CC3(C)C)cc2)c(C)n1]

To a mixture of 2,2-difluoro-[1,3]dioxolo[4,5-b]pyridin-6-amine (50.0 mg, 0.287 mmol) and N, N-diisopropylethylamine (185 mg, 1.43 mmol) in dichloromethane (6 mL) at 0° C. was added triphosgene (34 mg, 0.115 mmol). After 30 minutes, the reaction mixture was warmed to room temperature and methyl 5-(4-(3,3-dimethylazetidine-2-carboxamido)phenyl)-6-methylpicolinate (101 mg, 0.287 mmol) was added over 3 minutes. The resulting mixture was allowed to stir for an additional 2 hours. Upon completion, the reaction mixture was cooled to 0° C. and quenched with ice water. The resulting mixture was extracted with ethyl acetate (3×15 mL). The combined organic layers were dried over anhydrous sodium sulfate, filtered, and concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 25 min; detector: UV 254 nm) to afford methyl 5-(4-(1-((2,2-difluoro-[1,3]dioxolo[4,5-b]pyridin-6-yl)carbamoyl)-3,3-dimethylazetidine-2-carboxamido)phenyl)-6-methylpicolinate (40 mg, 25%) as a white solid. LCMS (ESI) [M+H]⁺: 554.2.

### Step 5: 5-(4-(1-((2,2-difluoro-[1,3]dioxolo[4,5-b]pyridin-6-yl)carbamoyl)-3,3-dimethylazetidine-2-carboxamido)phenyl)-6-methylpicolinic acid

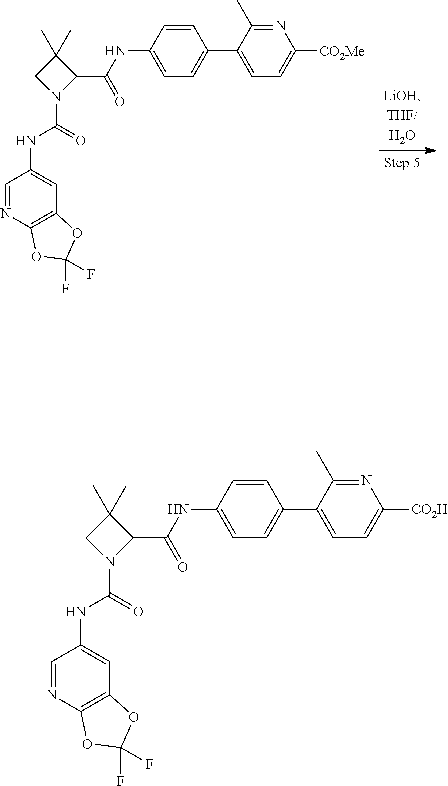 [SMILES: COC(=O)c1ccc(-c2ccc(NC(=O)C3N(C(=O)Nc4cnc5c(c4)OC(F)(F)O5)CC3(C)C)cc2)c(C)n1 ; Cc1nc(C(=O)O)ccc1-c1ccc(NC(=O)C2N(C(=O)Nc3cnc4c(c3)OC(F)(F)O4)CC2(C)C)cc1]

A solution of methyl 5-(4-(1-((2,2-difluoro-[1,3]dioxolo[4,5-b]pyridin-6-yl)carbamoyl)-3,3-dimethylazetidine-2-carboxamido)phenyl)-6-methylpicolinate (35.0 mg, 63.0 mmol) and lithium hydroxide monohydrate (8.0 mg, 0.189 mmol) in tetrahydrofuran (3.0 mL) and water (3.0 mL) was stirred at room temperature for 2 hours. Upon completion, the reaction mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 25 min; detector: UV 254 nm) to afford 5-(4-(1-((2,2-difluoro-[1,3]dioxolo[4,5-b]pyridin-6-yl)carbamoyl)-3,3-dimethylazetidine-2-carboxamido)phenyl)-6-methylpicolinic acid (24.0 mg, 70%) as a white solid. LCMS (ESI) [M+H]⁺: 540.2.

### Step 6: (S)-5-(4-(1-((2,2-difluoro-[1,3]dioxolo[4,5-b]pyridin-6-yl)carbamoyl)-3,3-dimethylazetidine-2-carboxamido)phenyl)-6-methylpicolinic acid and (R)-5-(4-(1-((2,2-difluoro-[1,3]dioxolo[4,5-b]pyridin-6-yl)carbamoyl)-3,3-dimethylazetidine-2-carboxamido)phenyl)-6-methylpicolinic acid

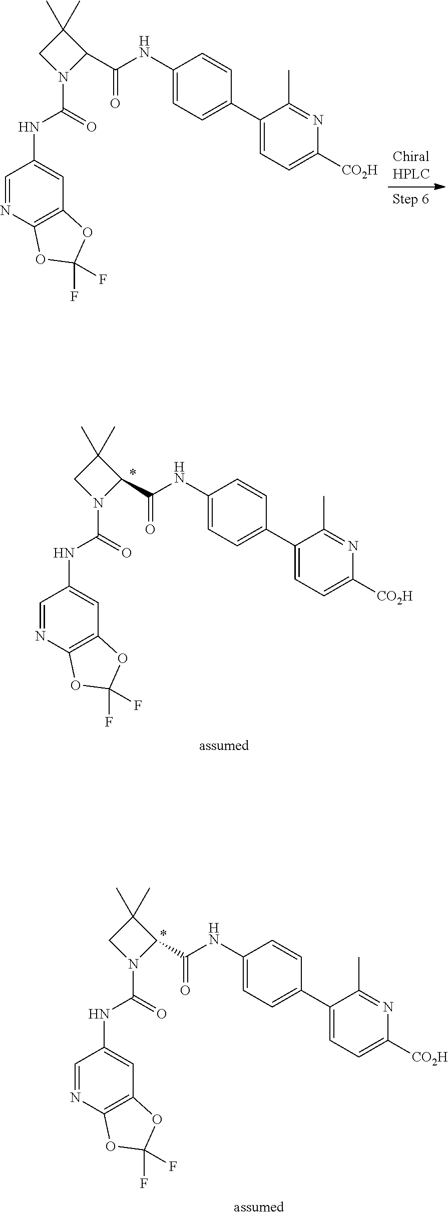 [SMILES: Cc1nc(C(=O)O)ccc1-c1ccc(NC(=O)C2N(C(=O)Nc3cnc4c(c3)OC(F)(F)O4)CC2(C)C)cc1 ; Cc1nc(C(=O)O)ccc1-c1ccc(NC(=O)[C@H]2N(C(=O)Nc3cnc4c(c3)OC(F)(F)O4)CC2(C)C)cc1 ; Cc1nc(C(=O)O)ccc1-c1ccc(NC(=O)[C@@H]2N(C(=O)Nc3cnc4c(c3)OC(F)(F)O4)CC2(C)C)cc1]

5-(4-(1-((2,2-difluoro-[1,3]dioxolo[4,5-b]pyridin-6-yl)carbamoyl)-3,3-dimethylazetidine-2-carboxamido)phenyl)-6-methylpicolinic acid (24 mg) was resolved by preparative chiral HPLC (conditions: column: CHIRAL ART Cellulose-SB, 3*25 cm, 5 μm; mobile phase A: hexane/MTBE (1:1, 0.1% formic acid); mobile phase B: methanol; flow rate: 40 mL/min; gradient (B %): isocratic 30% B; detector: 220/254 nm; RT1 (min): 5.3; RT2 (min): 13.6) to afford (Compound 196a; Method BA, Peak 1, 0.57 min) (S)-5-(4-(1-((2,2-difluoro-[1,3]dioxolo[4,5-b]pyridin-6-yl)carbamoyl)-3,3-dimethylazetidine-2-carboxamido)phenyl)-6-methylpicolinic acid (1^{st} peak, 7.9 mg, 33%) as a white solid and (Compound 196b; Method BA, Peak 2, 1.46 min) (R)-5-(4-(1-((2,2-difluoro-[1,3]dioxolo[4,5-b]pyridin-6-yl)carbamoyl)-3,3-dimethylazetidine-2-carboxamido)phenyl)-6-methylpicolinic acid (2^{nd} peak, 6.2 mg, 26%) as a white solid.

| Cmpd # | Characterization Data |
|---|---|
| 196a | LCMS (ESI) [M + H]⁺: 540.3 |
| | ¹H NMR (400 MHz, DMSO-d₆) δ 10.08 (s, 1H), 9.02 (s, 1H), 8.08 (d, J = 3.6 Hz, 2H), 7.94 |
| | (d, J = 8.0 Hz, 1H), 7.79-7.77 (m, 3H), 7.43 (d, J = 8.4 Hz, 2H), 4.45 (s, 1H), 3.97-3.86 |
| | (m, 1H), 3.79-3.77 (m, 1H), 2.41-2.39 (m, 3H), 1.43 (s, 3H), 1.24 (s, 3H). |
| 196b | LCMS (ESI) [M + H]⁺: 540.3 |
| | ¹H NMR (400 MHz, DMSO-d₆) δ 10.09 (s, 1H), 9.03 (s, 1H), 8.08 (d, J = 4.0 Hz, 2H), 7.93 |
| | (d, J = 7.6 Hz, 1H), 7.79-7.75 (m, 3H), 7.42 (d, J = 8.4 Hz, 2H), 4.51 (s, 1H), 3.79 (d, J = |
| | 7.6 Hz, 1H), 3.72 (d, J = 7.2 Hz, 1H), 2.46-2.39 (m, 3H), 1.54 (s, 3H), 1.15 (s, 3H). |

### Example 197: 4-(6-((2R,3R)-1-((2,2-difluorobenzo[d][1,3]dioxol-5-yl)carbamoyl)-3-methylpyrrolidin-2-yl)-5-fluoropyridin-3-yl)benzoic acid and 4-(6-((2S,3S)-1-((2,2-difluorobenzo[d][1,3]dioxol-5-yl)carbamoyl)-3-methylpyrrolidin-2-yl)-5-fluoropyridin-3-yl)benzoic acid

### Step 1: 1-(tert-butoxycarbonyl)-3-methyl-1H-pyrrole-2-carboxylic acid

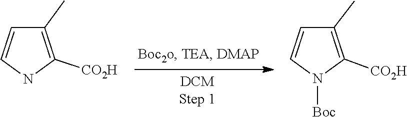

To 3-methyl-1H-pyrrole-2-carboxylic acid (4.00 g, 32.0 mmol) in dichloromethane (45 mL) at 0° C. were added sequentially Boc₂O (7.68 g, 35.2 mmol), DMAP (1.18 g, 9.59 mmol) and N, N-diisopropylethylamine (12.4 g, 95.9 mmol). The resultant mixture was warmed to room temperature and stirred for 16 hours. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %) 0% to 100% over 25 min; detector: UV 254 nm) to afford 1-(tert-butoxycarbonyl)-3-methyl-1H-pyrrole-2-carboxylic acid (3.00 g, 42% yield) as an off white solid. LCMS (ESI) [M−H]⁻: 224.1.

### Step 2: (+/−)-trans-1-(tert-butoxycarbonyl)-3-methylpyrrolidine-2-carboxylic acid

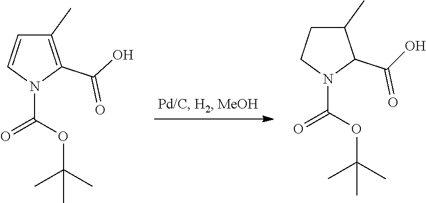 [SMILES: Cc1ccn(C(=O)OC(C)(C)C)c1C(=O)O ; CC1CCN(C(=O)OC(C)(C)C)C1C(=O)O]

A mixture of 1-(tert-butoxycarbonyl)-3-methyl-1H-pyrrole-2-carboxylic acid (3.00 g, 13.3 mmol) and 10% palladium on carbon (280 mg, 0.266 mmol) in methanol (40 mL) at room temperature was stirred overnight under an atmosphere of hydrogen (1 atm). Upon completion, the mixture was filtered through celite. The filter cake was washed with methanol (3×45 mL). The filtrate was concentrated under reduced pressure to afford (+/−)-trans-1-(tert-butoxycarbonyl)-3-methylpyrrolidine-2-carboxylic acid (2.20 g, crude) as a yellow oil, which was used directly in the next step without further purification. LCMS (ESI) [M−H]⁻: 228.1.

### Step 3: (+/−)-trans-tert-butyl 2-(5-(4-(tert-butoxycarbonyl)phenyl)-3-fluoropyridin-2-yl)-3-methylpyrrolidine-1-carboxylate

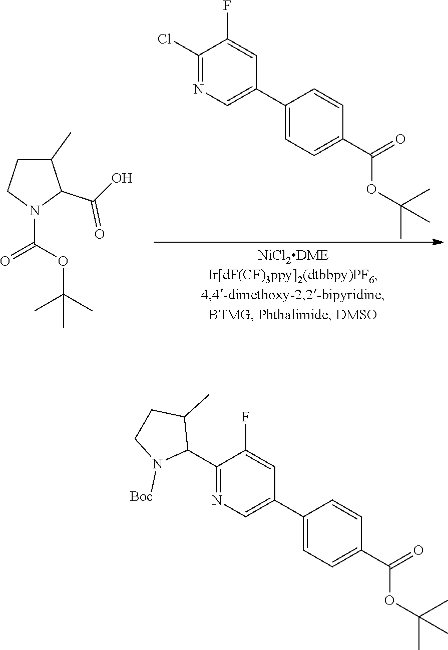 [SMILES: CC1CCN(C(=O)OC(C)(C)C)C1C(=O)O ; CC(C)(C)OC(=O)c1ccc(-c2cnc(Cl)c(F)c2)cc1 ; CC1CCN(C(=O)OC(C)(C)C)C1c1ncc(-c2ccc(C(=O)OC(C)(C)C)cc2)cc1F]

A solution of (+/−)-trans-1-(tert-butoxycarbonyl)-3-methylpyrrolidine-2-carboxylic acid (687 mg, 3.00 mmol), tert-butyl 4-(6-chloro-5-fluoropyridin-3-yl)benzoate (615 mg, 2.00 mmol), dichloronickel-1,2-dimethoxyethane (44.0 mg, 0.200 mmol), 4,4′-dimethoxy-2,2′-bipyridine (52.0 mg, 0.240 mmol), N″-tert-butyl-N,N,N′,N′-tetramethylguanidine (513 mg, 3.00 mmol), Ir[dF(CF₃)ppy]₂(dtbbpy))PF₆ (45 mg, 40.0 μmol) and 2,3-dihydro-1H-isoindole-1,3-dione (294 mg, 2.00 mmol) in DMSO (10.0 mL) at room temperature was stirred under 450 nm blue LED irradiation for 24 hours. Upon completion, the mixture was diluted with water and extracted with ethyl acetate (3×30 mL). The combined organic layers were washed with brine, dried over anhydrous sodium sulfate, filtered, and concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 25 min; detector: UV 254 nm) to afford (+/−)-trans-tert-butyl 2-(5-(4-(tert-butoxycarbonyl)phenyl)-3-fluoropyridin-2-yl)-3-methylpyrrolidine-1-carboxylate (150 mg, 16%) as an off-white solid. LCMS (ESI) [M+H]⁺: 457.2.

### Step 4: (+/−)-trans-4-(5-fluoro-6-(3-methylpyrrolidin-2-yl)pyridin-3-yl)benzoic acid

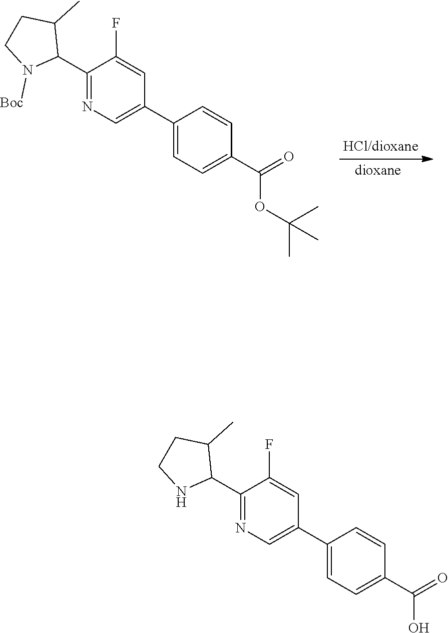 [SMILES: CC1CCN(C(=O)OC(C)(C)C)C1c1ncc(-c2ccc(C(=O)OC(C)(C)C)cc2)cc1F ; CC1CCNC1c1ncc(-c2ccc(C(=O)O)cc2)cc1F]

To a mixture of (+/−)-trans-tert-butyl 2-(5-(4-(tert-butoxycarbonyl)phenyl)-3-fluoropyridin-2-yl)-3-methylpyrrolidine-1-carboxylate (250 mg, 0.548 mmol) in 1,4-dioxane (1.0 mL) at 0° C. was added a solution of hydrochloric acid (5.0 mL, 20 mmol, 4.0 M in 1,4-dioxane) dropwise. The mixture was warmed to room temperature and stirred overnight. Upon completion, the mixture was concentrated under reduced pressure to afford (+/−)-trans-4-(5-fluoro-6-(3-methylpyrrolidin-2-yl)pyridin-3-yl)benzoic acid (160 mg, crude) as an off-white solid, which was used directly in the next step without further purification. LCMS (ESI) [M+H]⁺: 301.1.

### Step 5: (+/−)-trans-4-(6-(1-((2,2-difluorobenzo[d][1,3]dioxol-5-yl)carbamoyl)-3-methylpyrrolidin-2-yl)-5-fluoropyridin-3-yl)benzoic acid

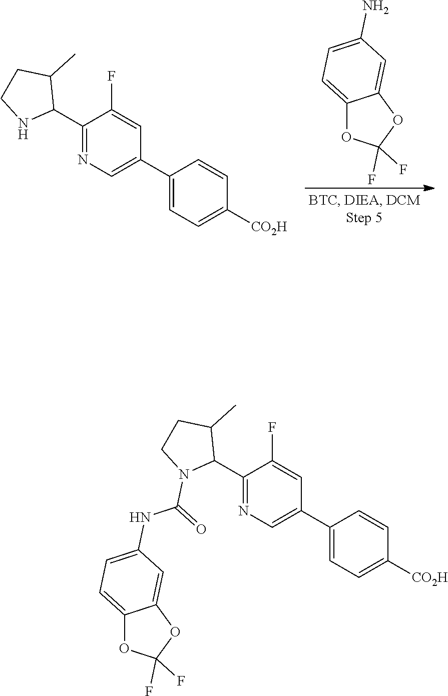 [SMILES: CC1CCNC1c1ncc(-c2ccc(C(=O)O)cc2)cc1F ; Nc1ccc2c(c1)OC(F)(F)O2 ; CC1CCN(C(=O)Nc2ccc3c(c2)OC(F)(F)O3)C1c1ncc(-c2ccc(C(=O)O)cc2)cc1F]

To 2,2-difluorobenzo[d][1,3]dioxol-5-amine (6.03 mg, 0.366 mmol) in dichloromethane (5.0 mL) at 0° C. were added N, N-diisopropylethylamine (237 mg, 1.83 mmol) followed by triphosgene (43 mg, 0.146 mmol). After 30 minutes, (+/−)-trans-4-(5-fluoro-6-(3-methylpyrrolidin-2-yl)pyridin-3-yl)benzoic acid (110 mg, 0.366 mmol) was added. The mixture was warmed to room temperature was allowed to stir for an additional 2 hours. Upon completion, the reaction was cooled in an ice bath and quenched with ice water. The mixture was extracted with ethyl acetate (3×15 mL). The combined organic layers were dried over anhydrous sodium sulfate, filtered, and concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 25 min; detector: UV 254 nm) to afford (+/−)-trans-4-(6-(1-((2,2-difluorobenzo[d][1,3]dioxol-5-yl)carbamoyl)-3-methylpyrrolidin-2-yl)-5-fluoropyridin-3-yl)benzoic acid (80 mg, 44%) as a yellow solid. LCMS (ESI) [M+H]⁺: 500.1.

### Step 6: 4-(6-((2R,3R)-1-((2,2-difluorobenzo[d][1,3]dioxol-5-yl)carbamoyl)-3-methylpyrrolidin-2-yl)-5-fluoropyridin-3-yl)benzoic acid and 4-(6-((2S,3S)-1-((2,2-difluorobenzo[d][1,3]dioxol-5-yl)carbamoyl)-3-methylpyrrolidin-2-yl)-5-fluoropyridin-3-yl)benzoic acid

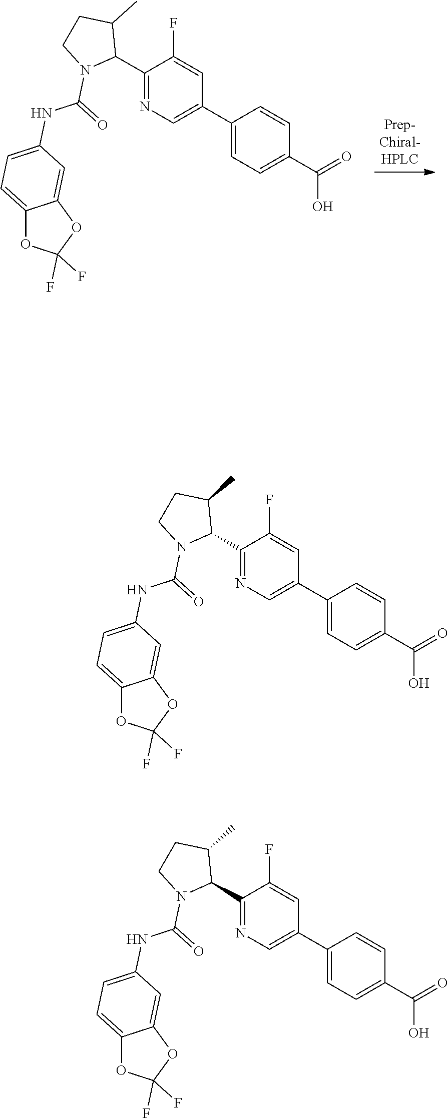 [SMILES: CC1CCN(C(=O)Nc2ccc3c(c2)OC(F)(F)O3)C1c1ncc(-c2ccc(C(=O)O)cc2)cc1F ; C[C@@H]1CCN(C(=O)Nc2ccc3c(c2)OC(F)(F)O3)[C@H]1c1ncc(-c2ccc(C(=O)O)cc2)cc1F ; C[C@H]1CCN(C(=O)Nc2ccc3c(c2)OC(F)(F)O3)[C@@H]1c1ncc(-c2ccc(C(=O)O)cc2)cc1F]

(+/−)-trans-4-(6-(1-((2,2-difluorobenzo[d][1,3]dioxol-5-yl)carbamoyl)-3-methylpyrrolidin-2-yl)-5-fluoropyridin-3-yl)benzoic acid (80 mg) was resolved by preparative chiral-HPLC (conditions: column: CHIRALPAK IK 3*25 cm, 5 μm; mobile phase A: hexane (0.1% formic acid); mobile phase B: isopropanol; flow rate: 40 mL/min; gradient (B %): isocratic 30% B; detector: 220/254 nm; RT1 (min): 9.3; RT2 (min): 13.5) to afford (Compound 197a; Method BB, Peak 1, 0.80 min) 4-(6-((2R,3R)-1-((2,2-difluorobenzo[d][1,3]dioxol-5-yl)carbamoyl)-3-methylpyrrolidin-2-yl)-5-fluoropyridin-3-yl)benzoic acid (1^{st} peak, 27.9 mg, 35%) as a white solid, and second peak (Compound 197b; Method BB, Peak 2, 1.14 min) 4-(6-((2S,3S)-1-((2,2-difluorobenzo[d][1,3]dioxol-5-yl)carbamoyl)-3-methylpyrrolidin-2-yl)-5-fluoropyridin-3-yl)benzoic acid (2^{nd} peak, 30.6 mg, 38%) as a white solid.

| Cmpd # | Characterization Data |
|---|---|
| 197a | LCMS (ESI) [M + H]⁺: 500.2 |
| | ¹H NMR (400 MHz, DMSO-d₆) δ 13.09 (bs, 1H), 8.75 (s, 1H), 8.49 (s, 1H), 8.09-7.89 (m, 5H), |
| | 7.55 (s, 1H), 7.23-7.15 (m, 2H), 4.85 (d, J = 5.2 Hz, 1H), 3.73 (s, 2H), 2.33 (t, J = 6.2 Hz, 1H), |
| | 2.22-2.20 (m, 1H), 1.75-1.68 (m, 1H), 1.13 (d, J = 6.8 Hz, 3H). |
| 197b | LCMS (ESI) [M + H]⁺: 500.2 |
| | ¹H NMR (400 MHz, DMSO-d₆) δ 13.08 (bs, 1H), 8.75 (s, 1H), 8.49 (s, 1H), 8.09-8.02 (m, 3H), |
| | 7.90 (d, J = 8.0 Hz, 2H), 7.55 (s, 1H), 7.23-7.15 (m, 2H), 4.85 (d, J = 6.4 Hz, 1H), 3.73-3.71 (m, |
| | 2H), 2.31-2.29 (m, 1H), 2.23-2.22 (m, 1H), 1.77-1.68 (m, 1H), 1.13 (d, J = 6.8 Hz, 3H). |

### Example 198: (R)-4-(6-(1-((2,2-difluorobenzo[d][1,3]dioxol-5-yl)carbamoyl)pyrrolidin-2-yl)-5-fluoropyridin-3-yl)benzoic acid and (S)-4-(6-(1-((2,2-difluorobenzo[d][1,3]dioxol-5-yl)carbamoyl)pyrrolidin-2-yl)-5-fluoropyridin-3-yl)benzoic acid

### Step 1: methyl 4-(6-(1-((2,2-difluorobenzo[d][1,3]dioxol-5-yl)carbamoyl)pyrrolidin-2-yl)-5-fluoropyridin-3-yl)benzoate

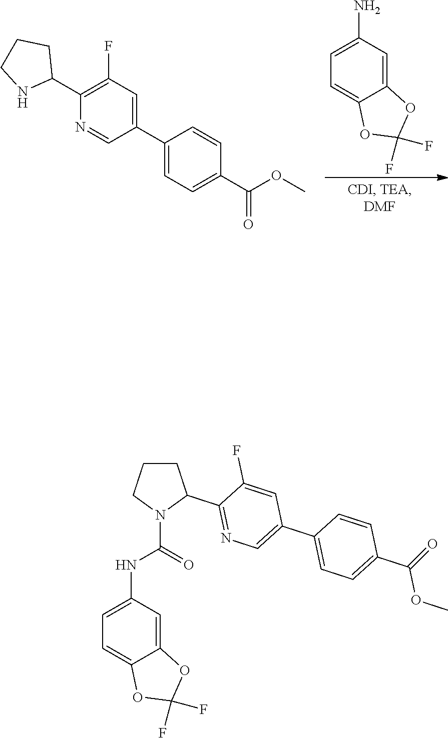 [SMILES: COC(=O)c1ccc(-c2cnc(C3CCCN3)c(F)c2)cc1 ; Nc1ccc2c(c1)OC(F)(F)O2 ; COC(=O)c1ccc(-c2cnc(C3CCCN3C(=O)Nc3ccc4c(c3)OC(F)(F)O4)c(F)c2)cc1]

Triethylamine (152 mg, 1.49 mmol) was added to a solution of 2,2-difluoro-1,3-benzodioxol-5-amine (259 mg, 1.49 mmol) and CDI (243 mg, 1.49 mmol) in DMF (3.0 mL) at room temperature. After 5 minutes, methyl 4-(5-fluoro-6-(pyrrolidin-2-yl)pyridin-3-yl)benzoate (150.0 mg, 0.49 mmol) was added and the mixture was stirred at 45° C. for 2 hours. Upon completion, the mixture was directly purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford methyl 4-(6-(1-((2,2-difluorobenzo[d][1,3]dioxol-5-yl)carbamoyl)pyrrolidin-2-yl)-5-fluoropyridin-3-yl)benzoate (170 mg, 68%) as an off-white solid. LCMS (ESI) [M+H]⁺: 500.1.

### Step 2: 4-(6-(5-((2,2-difluoro-[1,3]dioxolo[4,5-b]pyridin-5-yl)carbamoyl)-5-azaspiro[2.4]heptan-6-yl)-5-fluoropyridin-3-yl)benzoic acid

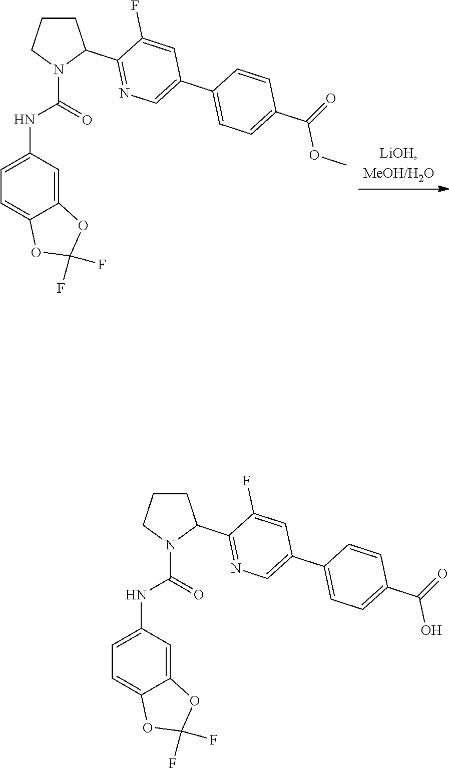 [SMILES: COC(=O)c1ccc(-c2cnc(C3CCCN3C(=O)Nc3ccc4c(c3)OC(F)(F)O4)c(F)c2)cc1 ; O=C(O)c1ccc(-c2cnc(C3CCCN3C(=O)Nc3ccc4c(c3)OC(F)(F)O4)c(F)c2)cc1]

A solution of methyl 4-(6-(1-((2,2-difluorobenzo[d][1,3]dioxol-5-yl)carbamoyl)pyrrolidin-2-yl)-5-fluoropyridin-3-yl)benzoate (160 mg, 0.32 mmol) and LiOH monohydroxide (269 mg, 6.40 mmol) in MeOH (2.0 mL) and H₂O (2.0 mL) was stirred at room temperature for 2 hours. Upon completion, the resulting mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford 4-(6-(5-((2,2-difluoro-[1,3]dioxolo[4,5-b]pyridin-5-yl)carbamoyl)-5-azaspiro[2.4]heptan-6-yl)-5-fluoropyridin-3-yl)benzoic acid (100 mg, 64%) as a white solid. LCMS (ESI) [M+H]⁺: 486.1.

### Step 3: (R)-4-(6-(1-((2,2-difluorobenzo[d][1,3]dioxol-5-yl)carbamoyl)pyrrolidin-2-yl)-5-fluoropyridin-3-yl)benzoic acid and (S)-4-(6-(1-((2,2-difluorobenzo[d][1,3]dioxol-5-yl)carbamoyl)pyrrolidin-2-yl)-5-fluoropyridin-3-yl)benzoic acid

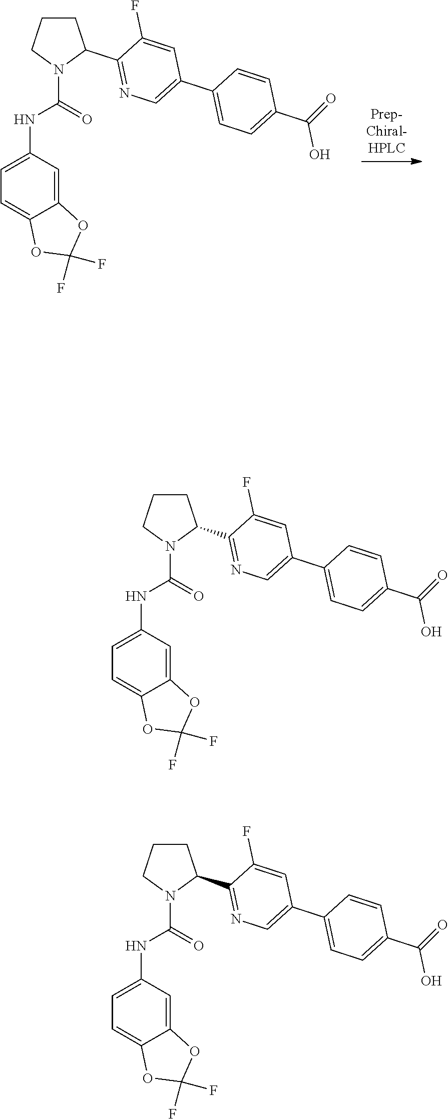 [SMILES: O=C(O)c1ccc(-c2cnc(C3CCCN3C(=O)Nc3ccc4c(c3)OC(F)(F)O4)c(F)c2)cc1 ; O=C(O)c1ccc(-c2cnc([C@H]3CCCN3C(=O)Nc3ccc4c(c3)OC(F)(F)O4)c(F)c2)cc1 ; O=C(O)c1ccc(-c2cnc([C@@H]3CCCN3C(=O)Nc3ccc4c(c3)OC(F)(F)O4)c(F)c2)cc1]

4-(6-(5-((2,2-difluoro-[1,3]dioxolo[4,5-b]pyridin-5-yl)carbamoyl)-5-azaspiro[2.4]heptan-6-yl)-5-fluoropyridin-3-yl)benzoic acid (100 mg) was resolved by preparative chiral HPLC (conditions: Column: Chiral NX(2) 5p 3*25 cm, 5 μm; Mobile Phase A: Hexane (0.1% FA), Mobile Phase B: EtOH; Flow rate: 40 mL/min; Gradient (B %): isocratic 40% B; Wave Length: 220/254 nm; RT1 (min): 7.33; RT2 (min): 17.77) to afford (Compound 85; Method AJ, Peak 1, 1.18 min) (R)-4-(6-(1-((2,2-difluorobenzo[d][1,3]dioxol-5-yl)carbamoyl)pyrrolidin-2-yl)-5-fluoropyridin-3-yl)benzoic acid (1^{st} peak, 16.2 mg, 40%) as a white solid, and (Compound 198; Method AJ, Peak 2, 2.57 min) (S)-4-(6-(1-((2,2-difluorobenzo[d][1,3]dioxol-5-yl)carbamoyl)pyrrolidin-2-yl)-5-fluoropyridin-3-yl)benzoic acid (2^{nd} peak, 19.4 mg, 48%) as a white solid.

| Cmpd # | Characterization Data |
|---|---|
| 85 | LCMS (ESI) [M + H]⁺: 486.2 |
| | ¹H NMR (400 MHz, DMSO-d₆) δ 13.02 (bs, 1H), 8.74 (d, J = 1.6 Hz, 1H), 8.48 (s, 1H), 8.08 (dd, J = |
| | 11.5, 1.9 Hz, 1H), 8.06-8.00 (m, 2H), 7.90 (d, J = 8.2 Hz, 2H), 7.58 (d, J = 2.0 Hz, 1H), 7.23 (d, |
| | J = 8.8 Hz, 1H), 7.18 (dd, J = 8.7, 2.1 Hz, 1H), 5.34 (dd, J = 8.0, 4.1 Hz, 1H), 3.79-3.71 (m, 1H), |
| | 3.68-3.58 (m, 1H), 2.41-2.30 (m, 1H), 2.20-1.85 (m, 3H) |
| 198 | LCMS (ESI) [M + H]⁺: 486.1 |
| | ¹H NMR (400 MHz, DMSO-d₆) δ 13.02 (bs, 1H), 8.74 (t, J = 1.5 Hz, 1H), 8.48 (s, 1H), 8.08 (dd, J = |
| | 11.5, 1.9 Hz, 1H), 8.03 (d, J = 8.3 Hz, 2H), 7.89 (d, J = 8.2 Hz, 2H), 7.58 (d, J = 2.0 Hz, 1H), |
| | 7.23 (d, J = 8.8 Hz, 1H), 7.18 (dd, J = 8.9, 2.0 Hz, 1H), 5.34 (dd, J = 7.7, 4.1 Hz, 1H), 3.81-3.70 |
| | (m, 1H), 3.69-3.60 (m, 1H), 2.40-2.30 (m, 1H), 2.18-1.87 (m, 3H). |

### Example 200: (R)-2-(3-fluoro-5-(1H-indazol-6-yl)pyridin-2-yl)-N-(4-isopropylphenyl)pyrrolidine-1-carboxamide

### Step 1: (R)-2-(3-fluoro-5-(1H-indazol-6-yl)pyridin-2-yl)-N-(4-isopropylphenyl)pyrrolidine-1-carboxamide

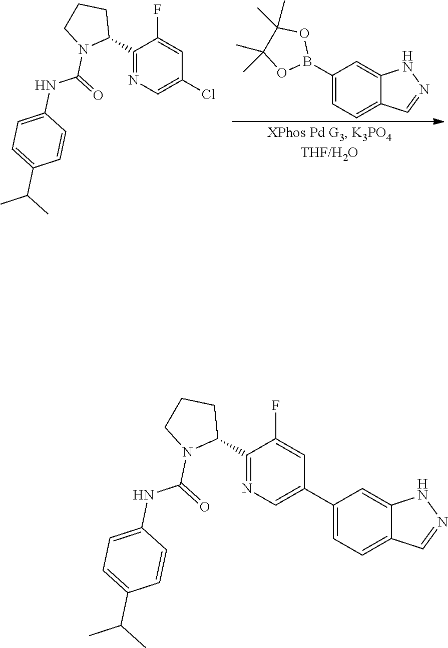 [SMILES: CC(C)c1ccc(NC(=O)N2CCC[C@@H]2c2ncc(Cl)cc2F)cc1 ; CC1(C)OB(c2ccc3cn[nH]c3c2)OC1(C)C ; CC(C)c1ccc(NC(=O)N2CCC[C@@H]2c2ncc(-c3ccc4cn[nH]c4c3)cc2F)cc1]

To a solution of (R)-2-(5-chloro-3-fluoropyridin-2-yl)-N-(4-isopropylphenyl)pyrrolidine-1-carboxamide (50.0 mg, 0.138 mmol), 6-(4,4,5,5-tetramethyl-1,3,2-dioxaborolan-2-yl)-1H-indazole (50.6 mg, 0.207 mmol), and tribasic potassium phosphate (88.0 mg, 0.414 mmol) was added XPhos Pd G3 (11.7 mg, 0.014 mmol) in tetrahydrofuran (1.5 mL) and water (0.50 mL). The reaction mixture was heated to 90° C. for 1 hour. Upon completion, the mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford (Compound 200) (R)-2-(3-fluoro-5-(1H-indazol-6-yl)pyridin-2-yl)-N-(4-isopropylphenyl)pyrrolidine-1-carboxamide (23.3 mg, 38%) as a white solid.

| Cmpd # | Characterization Data |
|---|---|
| 200 | LCMS (ESI) [M + H]⁺: 444.2 |
| | ¹H NMR (400 MHz, DMSO-d₆) δ 13.26 (s, 1H), 8.72 (s, 1H), 8.13 (d, J = 10.4 Hz, 2H), 8.04 (dd, |
| | J = 11.6, 1.9 Hz, 1H), 7.90-7.82 (m, 2H), 7.48 (dd, J = 8.4, 1.5 Hz, 1H), 7.34 (d, J = 8.5 Hz, |
| | 2H), 7.04 (d, J = 8.6 Hz, 2H), 5.39-5.29 (m, 1H), 3.85-3.70 (m, 1H), 3.69-3.53 (m, 1H), 2.78 |
| | (p, J = 6.9 Hz, 1H), 2.41-2.27 (m, 1H), 2.18-2.08 (m, 1H), 2.08-1.96 (m, 1H), 1.96-1.86 |
| | (m, 1H), 1.14 (d, J = 6.9 Hz, 6H). |

### Example 201: (R)-2-(3-fluoro-5-(4-oxo-3,4-dihydroquinazolin-7-yl)pyridin-2-yl)-N-(4-isopropylphenyl)pyrrolidine-1-carboxamide

### Step 1: 7-bromo-3-((2-(trimethylsilyl)ethoxy)methyl)quinazolin-4(3H)-one

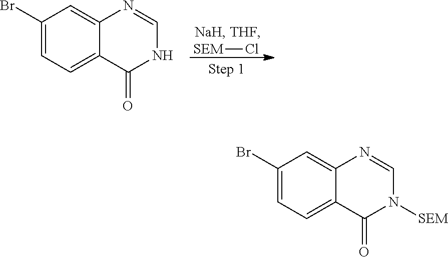 [SMILES: O=c1[nH]cnc2cc(Br)ccc12 ; C[Si](C)(C)CCOCCl ; C[Si](C)(C)CCOCn1cnc2cc(Br)ccc2c1=O]

To a solution of 7-bromo-3H-quinazolin-4-one (300 mg, 1.33 mmol) in tetrahydrofuran (5.0 mL) at 0° C. was added sodium hydride (60% in mineral oil, 96.0 mg, 4.00 mmol). After 30 minutes neat [2-(chloromethoxy)ethyl]trimethylsilane (444 mg, 2.67 mmol), was added dropwise. The resultant mixture was warmed to room temperature and stirred for 1.0 hour. Upon completion, the solvent was removed under reduced pressure. The crude residue was directly purified by silica gel column chromatography, eluted with petroleum ether/ethyl acetate (1:1) to afford 7-bromo-3-((2-(trimethylsilyl)ethoxy)methyl)quinazolin-4(3H)-one (85.0 mg, 18%) as a yellow oil. LCMS (ESI) [M+H]⁺: 357.0.

### Step 2: (R)-2-(3-fluoro-5-(4-oxo-3-((2-(trimethylsilyl)ethoxy)methyl)-3,4-dihydroquinazolin-7-yl)pyridin-2-yl)-N-(4-isopropylphenyl)pyrrolidine-1-carboxamide

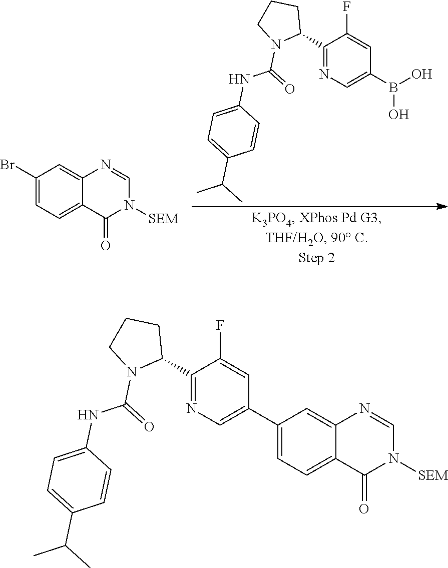 [SMILES: C[Si](C)(C)CCOCn1cnc2cc(Br)ccc2c1=O ; CC(C)c1ccc(NC(=O)N2CCC[C@@H]2c2ncc(B(O)O)cc2F)cc1 ; CC(C)c1ccc(NC(=O)N2CCC[C@@H]2c2ncc(-c3ccc4c(=O)n(COCC[Si](C)(C)C)cnc4c3)cc2F)cc1]

To a solution of 7-bromo-3-((2-(trimethylsilyl)ethoxy)methyl)quinazolin-4(3H)-one (73.0 mg, 0.205 mmol), (R)-(5-fluoro-6-(1-((4-isopropylphenyl)carbamoyl)pyrrolidin-2-yl)pyridin-3-yl)boronic acid (76.0 mg, 0.205 mmol), and tribasic potassium phosphate (87.2 mg, 0.410 mmol) in tetrahydrofuran (4.0 mL) and water (1.0 mL) was added XPhos Pd G3 (17.4 mg, 0.021 mmol). The resultant mixture was stirred at 90° C. for 1 hour. Upon completion, the mixture was concentrated under reduced pressure. The crude residue was directly purified by silica gel column chromatography, eluted with petroleum ether/ethyl acetate (1:1), to afford (R)-2-(3-fluoro-5-(4-oxo-3-((2-(trimethylsilyl)ethoxy)methyl)-3,4-dihydroquinazolin-7-yl)pyridin-2-yl)-N-(4-isopropylphenyl)pyrrolidine-1-carboxamide (40.0 mg, 32%) as a yellow oil. LCMS (ESI) [M+H]⁺: 602.2.

### Step 3: (R)-2-(3-fluoro-5-(4-oxo-3,4-dihydroquinazolin-7-yl)pyridin-2-yl)-N-(4-isopropylphenyl)pyrrolidine-1-carboxamide

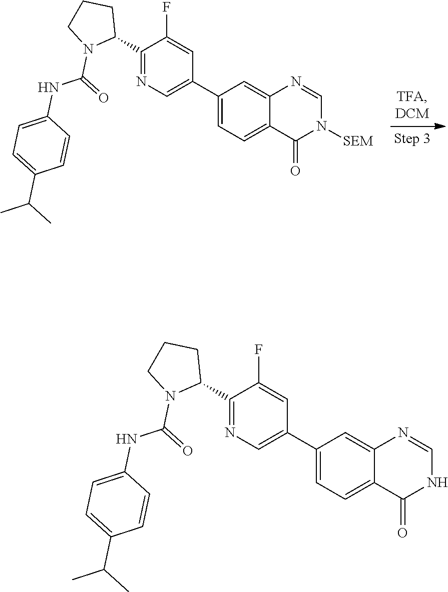 [SMILES: CC(C)c1ccc(NC(=O)N2CCC[C@@H]2c2ncc(-c3ccc4c(=O)n(COCC[Si](C)(C)C)cnc4c3)cc2F)cc1 ; CC(C)c1ccc(NC(=O)N2CCC[C@@H]2c2ncc(-c3ccc4c(=O)[nH]cnc4c3)cc2F)cc1]

To a solution of (R)-2-(3-fluoro-5-(4-oxo-3-((2-(trimethylsilyl)ethoxy)methyl)-3,4-dihydroquinazolin-7-yl)pyridin-2-yl)-N-(4-isopropylphenyl)pyrrolidine-1-carboxamide (30.0 mg, 0.050 mmol) in dichloromethane (2.5 mL) at room temperature was added trifluoroacetic acid (2.5 mL). After 3 hours, the mixture was concentrated under reduced pressure. The crude residue was purified by preparative HPLC (conditions: column: XSelect CSH Fluoro Phenyl 30*150 mm, 5 μm; mobile phase A: water (0.1% formic acid), mobile phase B: acetonitrile; flow rate: 60 mL/min; gradient (B %): 31% to 45% B over 15 min; detector: 254 nm/220 nm; RT (min): 9.9) to afford (Compound 201) (R)-2-(3-fluoro-5-(4-oxo-3,4-dihydroquinazolin-7-yl)pyridin-2-yl)-N-(4-isopropylphenyl)pyrrolidine-1-carboxamide (10.3 mg, 44%) as a white solid.

| Cmpd # | Characterization Data |
|---|---|
| 201 | LCMS (ESI) [M + H]⁺: 472.3 |
| | ¹H NMR (400 MHz, DMSO-d₆) δ 12.33 (bs, 1H), 8.81 (s, 1H), 8.25-8.09 (m, 4H), 8.06 (d, J = |
| | 1.8 Hz, 1H), 7.91 (dd, J = 8.3, 1.9 Hz, 1H), 7.34 (d, J = 8.2 Hz, 2H), 7.04 (d, J = 8.3 Hz, 2H), 5.41- |
| | 5.27 (m, 1H), 3.84-3.71 (m, 1H), 3.69-3.56 (m, 1H), 2.78 (hept, J = 6.9 Hz, 1H), 2.41-2.27 |
| | (m, 1H), 2.19-2.08 (m, 1H), 2.08-1.97 (m, 1H), 1.97-1.85 (m, 1H), 1.14 (d, J = 6.8 Hz, 6H). |

### Example 202: (R)-2-(3-fluoro-5-(4-oxo-3,4-dihydroquinazolin-6-yl)pyridin-2-yl)-N-(4-isopropylphenyl)pyrrolidine-1-carboxamide

### Step 1: 7-bromo-2-((2-(trimethylsilyl)ethoxy)methyl)phthalazin-1(2H)-one

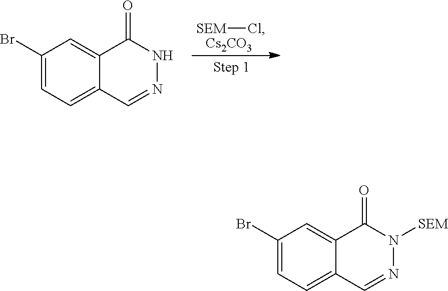

A mixture of 7-bromo-2H-phthalazin-1-one (200 mg, 0.889 mmol), [2-(chloromethoxy)ethyl]trimethylsilane (296 mg, 1.78 mmol) and cesium carbonate (869 mg, 2.67 mmol) in tetrahydrofuran (4.0 mL) was stirred for 1 hour at room temperature. Upon completion, the mixture was concentrated under reduced pressure. The crude residue was directly purified by silica gel column chromatography, eluted with petroleum ether/ethyl acetate (1:1) to afford 7-bromo-2-((2-(trimethylsilyl)ethoxy)methyl)phthalazin-1(2H)-one (100 mg, 32%) as a yellow oil. LCMS (ESI) [M+H]⁺: 355.3.

### Step 2: (R)-2-(3-fluoro-5-(4-oxo-3-((2-(trimethylsilyl)ethoxy)methyl)-3,4-dihydrophthalazin-6-yl)pyridin-2-yl)-N-(4-isopropylphenyl)pyrrolidine-1-carboxamide

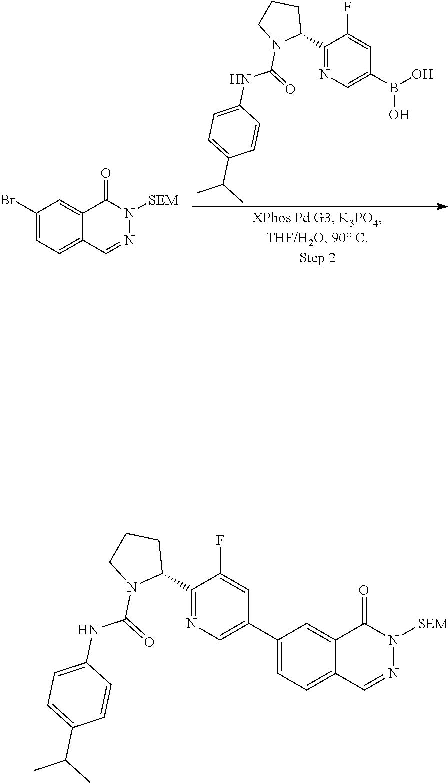 [SMILES: C[Si](C)(C)CCOCn1ncc2ccc(Br)cc2c1=O ; CC(C)c1ccc(NC(=O)N2CCC[C@@H]2c2ncc(B(O)O)cc2F)cc1 ; CC(C)c1ccc(NC(=O)N2CCC[C@@H]2c2ncc(-c3ccc4cnn(COCC[Si](C)(C)C)c(=O)c4c3)cc2F)cc1]

To a solution of (R)-(5-fluoro-6-(1-((4-isopropylphenyl)carbamoyl)pyrrolidin-2-yl)pyridin-3-yl)boronic acid (74.9 mg, 0.203 mmol), 7-bromo-2-((2-(trimethylsilyl)ethoxy)methyl)phthalazin-1(2H)-one (71.7 mg, 0.203 mmol), and tribasic potassium phosphate (85.8 mg, 0.405 mmol) in tetrahydrofuran (1.0 mL) and water (0.50 mL) was added XPhos Pd G3 (22.8 mg, 0.027 mmol). The resultant mixture was heated to 90° C. for 1 hour. Upon completion, the mixture was concentrated under reduced pressure. The crude residue was directly purified by silica chromatography, eluted with petroleum ether/ethyl acetate (1:1), to afford (R)-2-(3-fluoro-5-(4-oxo-3-((2-(trimethylsilyl)ethoxy)methyl)-3,4-dihydrophthalazin-6-yl)pyridin-2-yl)-N-(4-isopropylphenyl)pyrrolidine-1-carboxamide (50 mg, 62%) as a yellow-green solid. LCMS (ESI) [M+H]⁺: 602.8.

### Step 3: (R)-2-(3-fluoro-5-(4-oxo-3,4-dihydroquinazolin-6-yl)pyridin-2-yl)-N-(4-isopropylphenyl)pyrrolidine-1-carboxamide

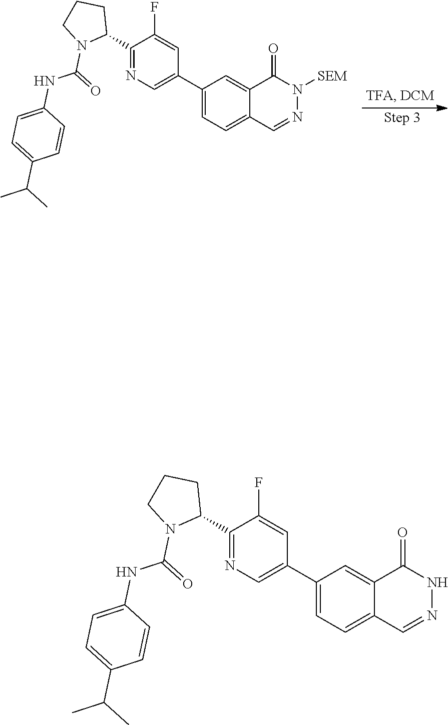 [SMILES: CC(C)c1ccc(NC(=O)N2CCC[C@@H]2c2ncc(-c3ccc4cnn(COCC[Si](C)(C)C)c(=O)c4c3)cc2F)cc1 ; CC(C)c1ccc(NC(=O)N2CCC[C@@H]2c2ncc(-c3ccc4cn[nH]c(=O)c4c3)cc2F)cc1]

To a solution of (R)-2-(3-fluoro-5-(4-oxo-3-((2-(trimethylsilyl)ethoxy)methyl)-3,4-dihydrophthalazin-6-yl)pyridin-2-yl)-N-(4-isopropylphenyl)pyrrolidine-1-carboxamide (50.0 mg, 0.083 mmol) in dichloromethane (2.0 mL) at room temperature was added trifluoroacetic acid (1.0 mL). After 3 hours, the mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford (Compound 202) (R)-2-(3-fluoro-5-(4-oxo-3,4-dihydroquinazolin-6-yl)pyridin-2-yl)-N-(4-isopropylphenyl)pyrrolidine-1-carboxamide (23.6 mg, 60%) as a white solid.

| Cmpd # | Characterization Data |
|---|---|
| 202 | LCMS (ESI) [M + H]⁺: 472.2 |
| | ¹H NMR (400 MHz, DMSO-d₆) δ 12.39 (bs, 1H), 8.75 (s, 1H), 8.41 (d, J = 2.3 Hz, 1H), 8.26- |
| | 8.05 (m, 4H), 7.77 (d, J = 8.5 Hz, 1H), 7.34 (d, J = 8.2 Hz, 2H), 7.04 (d, J = 8.2 Hz, 2H), 5.41- |
| | 5.29 (m, 1H), 3.84-3.70 (m, 1H), 3.70-3.57 (m, 1H), 2.78 (hept, J = 6.9 Hz, 1H), 2.43-2.30 |
| | (m, 1H), 2.19-2.07 (m, 1H), 2.07-1.95 (m, 1H), 1.95-1.83 (m, 1H), 1.14 (d, J = 6.9 Hz, 6H). |

### Example 203: (R)-2-(3-fluoro-5-(4-oxo-3,4-dihydroquinazolin-6-yl)pyridin-2-yl)-N-(4-isopropylphenyl)pyrrolidine-1-carboxamide

### Step 1: 6-bromo-3-((2-(trimethylsilyl)ethoxy)methyl)quinazolin-4(3H)-one

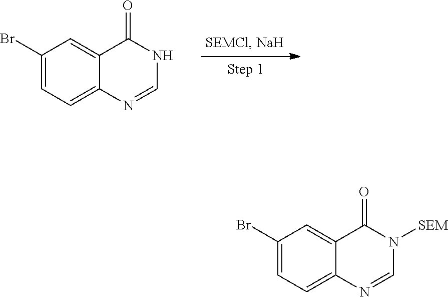 [SMILES: O=c1[nH]cnc2ccc(Br)cc12 ; C[Si](C)(C)CCOCn1cnc2ccc(Br)cc2c1=O]

To a solution of 6-bromoquinazolin-4(31H)-one (200 mg, 0.889 mmol) and [2-(chloromethoxy)ethyl]trimethylsilane (296 mg, 1.78 mmol) in tetrahydrofuran (4.0 mL) at room temperature was added sodium hydride (60% in mineral oil, 21.3 mg, 0.889 mmol). After 4 hours, the mixture was concentrated under reduced pressure. The crude residue was purified by silica gel chromatography, eluted with petroleum ether/ethyl acetate (1:1), to afford 6-bromo-3-((2-(trimethylsilyl)ethoxy)methyl)quinazolin-4(3H)-one (120 mg, 38%) as a yellow oil. LCMS (ESI) [M+H]⁺: 355.3.

### Step 2: (R)-2-(3-fluoro-5-(4-oxo-3-((2-(trimethylsilyl)ethoxy)methyl)-3,4-dihydroquinazolin-6-yl)pyridin-2-yl)-N-(4-isopropylphenyl)pyrrolidine-1-carboxamide

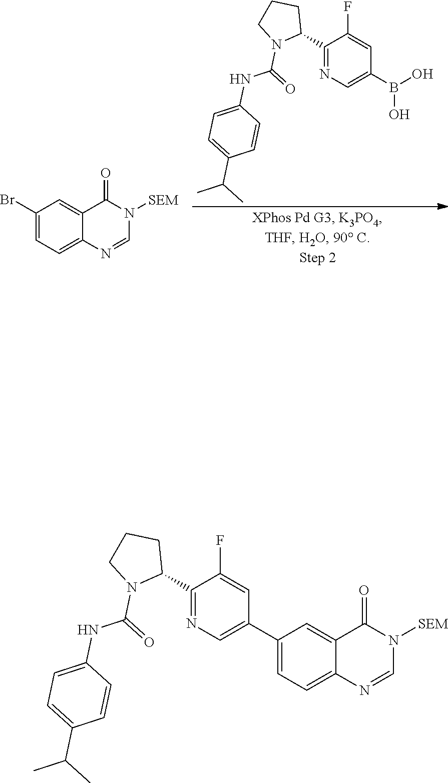 [SMILES: C[Si](C)(C)CCOCn1cnc2ccc(Br)cc2c1=O ; CC(C)c1ccc(NC(=O)N2CCC[C@@H]2c2ncc(B(O)O)cc2F)cc1 ; CC(C)c1ccc(NC(=O)N2CCC[C@@H]2c2ncc(-c3ccc4ncn(COCC[Si](C)(C)C)c(=O)c4c3)cc2F)cc1]

To a solution of (R)-(5-fluoro-6-(1-((4-isopropylphenyl)carbamoyl)pyrrolidin-2-yl)pyridin-3-yl)boronic acid (74.9 mg, 0.203 mmol), 6-bromo-3-((2-(trimethylsilyl)ethoxy)methyl)quinazolin-4(3H)-one (71.7 mg, 0.203 mmol), and tribasic potassium phosphate (85.8 mg, 0.405 mmol) in tetrahydrofuran (1.0 mL) and water (0.50 mL) was added XPhos Pd G3 (22.8 mg, 0.027 mmol). The resultant mixture was heated to 90° C. for 1 hour. Upon completion, the mixture was concentrated under reduced pressure. The crude residue was directly purified by silica gel column chromatography, eluted with petroleum ether/ethyl acetate (1:1), to afford (R)-2-(3-fluoro-5-(4-oxo-3-((2-(trimethylsilyl)ethoxy)methyl)-3,4-dihydroquinazolin-6-yl)pyridin-2-yl)-N-(4-isopropylphenyl)pyrrolidine-1-carboxamide (47.0 mg, 60%) as a yellow-green solid. LCMS (ESI) [M+H]⁺: 602.8.

### Step 3: (R)-2-(3-fluoro-5-(4-oxo-3,4-dihydroquinazolin-6-yl)pyridin-2-yl)-N-(4-isopropylphenyl)pyrrolidine-1-carboxamide

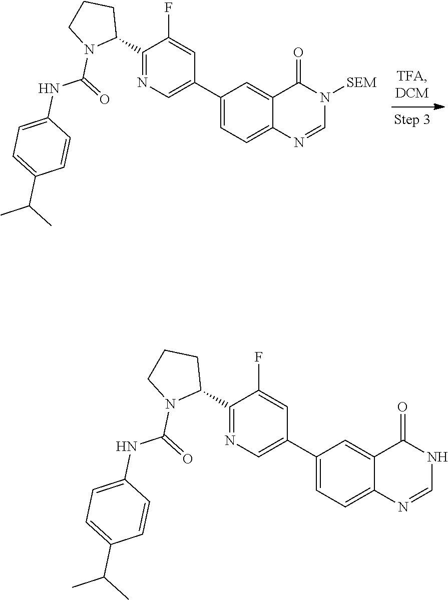 [SMILES: CC(C)c1ccc(NC(=O)N2CCC[C@@H]2c2ncc(-c3ccc4ncn(COCC[Si](C)(C)C)c(=O)c4c3)cc2F)cc1 ; CC(C)c1ccc(NC(=O)N2CCC[C@@H]2c2ncc(-c3ccc4nc[nH]c(=O)c4c3)cc2F)cc1]

To a solution of (R)-2-(3-fluoro-5-(4-oxo-3-((2-(trimethylsilyl)ethoxy)methyl)-3,4-dihydroquinazolin-6-yl)pyridin-2-yl)-N-(4-isopropylphenyl)pyrrolidine-1-carboxamide (47.0 mg, 0.078 mmol) in dichloromethane (2.0 mL) at room temperature was added trifluoroacetic acid (1.0 mL). After 3 hours, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford (Compound 203) (R)-2-(3-fluoro-5-(4-oxo-3,4-dihydroquinazolin-6-yl)pyridin-2-yl)-N-(4-isopropylphenyl)pyrrolidine-1-carboxamide (22.4 mg, 61%) as a white solid.

| Cmpd # | Characterization Data |
|---|---|
| 203 | LCMS (ESI) [M + H]⁺: 472.2 |
| | ¹H NMR (400 MHz, DMSO-d₆) δ 12.76 (s, 1H), 8.81 (s, 1H), 8.58-8.46 (m, 1H), 8.42 (s, 1H), |
| | 8.38-8.29 (m, 1H), 8.24-8.13 (m, 2H), 8.10-8.01 (m, 1H), 7.34 (d, J = 8.3 Hz, 2H), 7.04 (d, |
| | J = 8.6 Hz, 2H), 5.40-5.32 (m, 1H), 3.84-3.70 (m, 1H), 3.70-3.55 (m, 1H), 2.77 (hept, J = |
| | 6.9 Hz, 1H), 2.43-2.27 (m, 1H), 2.23-2.07 (m, 1H), 2.09-1.97 (m, 1H), 1.97-1.85 (m, 1H), |
| | 1.14 (d, J = 6.9 Hz, 6H). |

### Example 204: (R)-2-(5-(1H-[1,2,3]triazolo[4,5-b]pyridin-5-yl)-3-fluoropyridin-2-yl)-N-(4-isopropylphenyl)pyrrolidine-1-carboxamide

### Step 1: 5-chloro-1-((2-(trimethylsilyl)ethoxy)methyl)-1H-[1,2,3]triazolo[4,5-b]pyridine

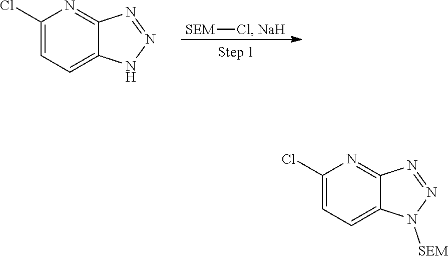

To 5-chloro-1H-[1,2,3]triazolo[4,5-b]pyridine (600 mg, 3.88 mmol) and [2-(chloromethoxy)ethyl]trimethylsilane (777 mg, 4.65 mmol) in tetrahydrofuran (8.0 mL) at room temperature was added and sodium hydride (60% in mineral oil, 466 mg, 11.6 mmol). After 2 hours, the mixture was concentrated under reduced pressure. The crude residue was directly purified by silica gel column chromatography, eluted with petroleum ether/ethyl acetate (1:1), to afford 5-chloro-1-((2-(trimethylsilyl)ethoxy)methyl)-1H-[1,2,3]triazolo[4,5-b]pyridine (500 mg, 45%) as a yellow oil. LCMS (ESI) [M+H]⁺: 285.2.

### Step 2: (R)-2-(3-fluoro-5-(1-((2-(trimethylsilyl)ethoxy)methyl)-1H-[1,2,3]triazolo[4,5-b]pyridin-5-yl)pyridin-2-yl)-N-(4-isopropylphenyl)pyrrolidine-1-carboxamide

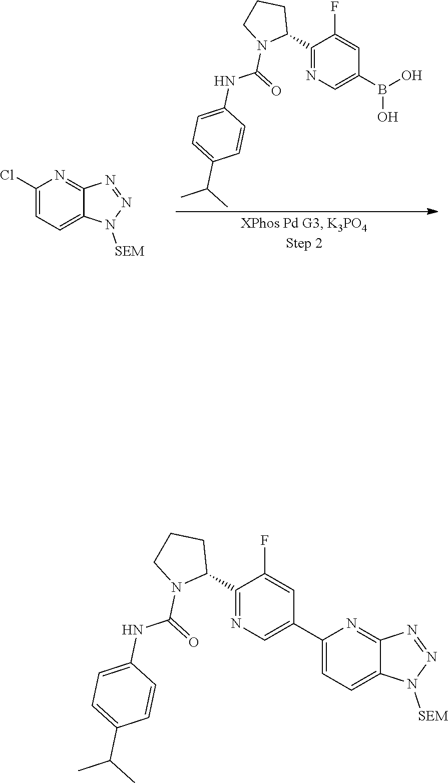 [SMILES: C[Si](C)(C)CCOCn1nnc2nc(Cl)ccc21 ; CC(C)c1ccc(NC(=O)N2CCC[C@@H]2c2ncc(B(O)O)cc2F)cc1 ; CC(C)c1ccc(NC(=O)N2CCC[C@@H]2c2ncc(-c3ccc4c(nnn4COCC[Si](C)(C)C)n3)cc2F)cc1]

To solution of (R)-(5-fluoro-6-(1-((4-isopropylphenyl)carbamoyl)pyrrolidin-2-yl)pyridin-3-yl)boronic acid (67.2 mg, 0.148 mmol), 5-chloro-1-((2-(trimethylsilyl)ethoxy)methyl)-1H-[1,2,3]triazolo[4,5-b]pyridine (42.2 mg, 0.140 mmol), and tribasic potassium phosphate (85.8 mg, 0.405 mmol) in tetrahydrofuran (1.0 mL) and water (0.50 mL) was added XPhos Pd G3 (22.8 mg, 0.027 mmol). The resultant mixture was heated to 90° C. for 1 hour. Upon completion, the mixture was cooled to ambient temperature and concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 20 min; detector: UV 254 nm) to afford (R)-2-(3-fluoro-5-(1-((2-(trimethylsilyl)ethoxy)methyl)-1H-[1,2,3]triazolo[4,5-b]pyridin-5-yl)pyridin-2-yl)-N-(4-isopropylphenyl)pyrrolidine-1-carboxamide (55 mg, 70%) as a pale yellow oil. LCMS (ESI) [M+H]⁺: 576.2.

### Step 3: (R)-2-(5-(1H-[1,2,3]triazolo[4,5-b]pyridin-5-yl)-3-fluoropyridin-2-yl)-N-(4-isopropylphenyl)pyrrolidine-1-carboxamide

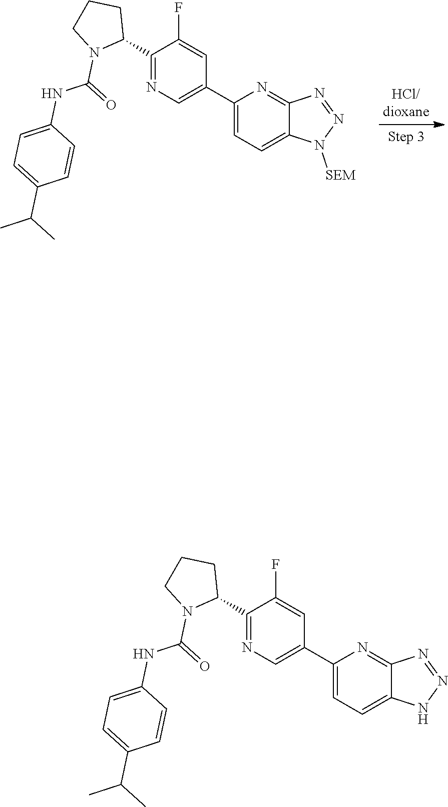 [SMILES: CC(C)c1ccc(NC(=O)N2CCC[C@@H]2c2ncc(-c3ccc4c(nnn4COCC[Si](C)(C)C)n3)cc2F)cc1 ; CC(C)c1ccc(NC(=O)N2CCC[C@@H]2c2ncc(-c3ccc4[nH]nnc4n3)cc2F)cc1]

To a solution of (R)-2-(3-fluoro-5-(1-((2-(trimethylsilyl)ethoxy)methyl)-1H-[1,2,3]triazolo[4,5-b]pyridin-5-yl)pyridin-2-yl)-N-(4-isopropylphenyl)pyrrolidine-1-carboxamide (55.0 mg, 0.090 mmol) in 1,4-dioxane (4.0 mL) at room temperature was added a solution of hydrochloric acid (2.4 mL, 9.60 mmol, 4.0 M in 1,4-dioxane). After 3 hours, the mixture was concentrated under reduced pressure. The crude residue was directly purified by preparative HPLC (conditions: column: Xselect CSH Prep C18, 30*150 mm 5 μm; mobile phase A: water (0.1% formic acid), mobile phase B: acetonitrile; Flow rate: 60 mL/min; gradient (B %): 50% to 64% over 20 min; detector: 254 nm/220 nm; RT (min): 19.1) to afford (Compound 204) (R)-2-(5-(1H-[1,2,3]triazolo[4,5-b]pyridin-5-yl)-3-fluoropyridin-2-yl)-N-(4-isopropylphenyl)pyrrolidine-1-carboxamide (12.7 mg, 29%) as a white solid.

| Cmpd # | Characterization Data |
|---|---|
| 204 | LCMS (ESI) [M + H]⁺: 446.2 |
| | ¹H NMR (400 MHz, DMSO-d₆) δ 9.13 (s, 1H), 8.58 (d, J = 8.8 Hz, 1H), 8.36 (d, J = 11.4 Hz, |
| | 1H), 8.18 (d, J = 11.1 Hz, 2H), 7.33 (d, J = 8.1 Hz, 2H), 7.04 (d, J = 8.4 Hz, 2H), 5.39-5.33 (m, |
| | 1H), 3.80-3.75 (m, 1H), 3.66-3.62 (m, 1H), 2.80-2.75 (m, 1H), 2.40-2.33 (m, 1H), 2.16- |
| | 2.10 (m, 1H), 2.06-2.00 (m, 1H), 1.94-1.88 (m, 1H), 1.14 (d, J = 6.9 Hz, 6H). |

### Example 205: (S)-4-(5-fluoro-6-(1-((4-methyl-5-(1-(trifluoromethyl)cyclopropyl)pyridin-2-yl)carbamoyl)piperidin-2-yl)pyridin-3-yl)benzoic acid and (R)-4-(5-fluoro-6-(1-((4-methyl-5-(1-(trifluoromethyl)cyclopropyl)pyridin-2-yl)carbamoyl)piperidin-2-yl)pyridin-3-yl)benzoic acid

### Step 1: methyl 4-(5-fluoro-6-(1-((4-methyl-5-(1-(trifluoromethyl)cyclopropyl)pyridin-2-yl)carbamoyl)piperidin-2-yl)pyridin-3-yl)benzoate

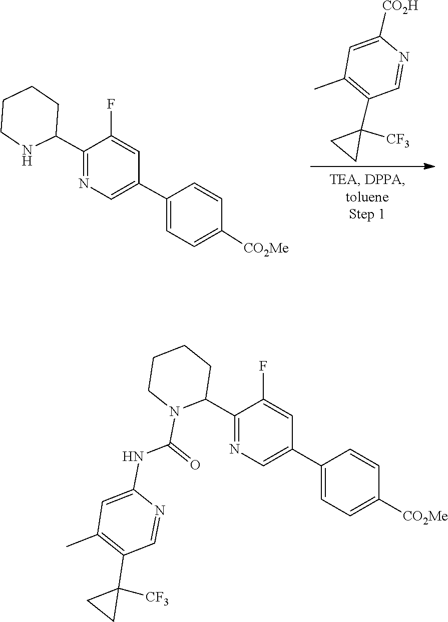 [SMILES: COC(=O)c1ccc(-c2cnc(C3CCCCN3)c(F)c2)cc1 ; Cc1cc(C(=O)O)ncc1C1(C(F)(F)F)CC1 ; COC(=O)c1ccc(-c2cnc(C3CCCCN3C(=O)Nc3cc(C)c(C4(C(F)(F)F)CC4)cn3)c(F)c2)cc1]

To a solution of methyl 4-[5-fluoro-6-(piperidin-2-yl)pyridin-3-yl]benzoate (200 mg, 0.630 mmol), in toluene (2.0 mL) was added 4-methyl-5-[l-(trifluoromethyl)cyclopropyl]pyridine-2-carboxylic acid (156 mg, 0.630 mmol), diphenylphosphoryl azide (525 mg, 1.90 mmol), and triethylamine (193 mg, 1.90 mmol). The resulting mixture was heated to 80° C. for 2 hours. Upon completion, the mixture was concentrated under reduced pressure. The crude residue was directly purified by silica gel column chromatography, eluted with petroleum ether/ethyl acetate (1:1) to afford methyl 4-(5-fluoro-6-(1-((4-methyl-5-(1-(trifluoromethyl)cyclopropyl)pyridin-2-yl)carbamoyl)piperidin-2-yl)pyridin-3-yl)benzoate (70 mg, 20%) as a white solid. LCMS (ESI) [M+H]⁺: 557.2.

### Step 2: 4-(5-fluoro-6-(1-((4-methyl-5-(1-(trifluoromethyl)cyclopropyl)pyridin-2-yl)carbamoyl)piperidin-2-yl)pyridin-3-yl)benzoic acid

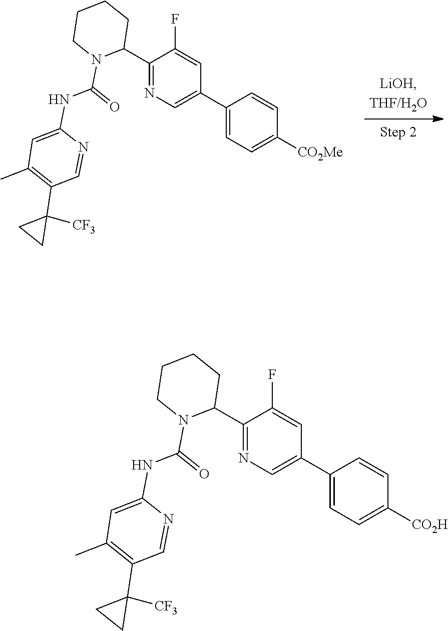 [SMILES: COC(=O)c1ccc(-c2cnc(C3CCCCN3C(=O)Nc3cc(C)c(C4(C(F)(F)F)CC4)cn3)c(F)c2)cc1 ; Cc1cc(NC(=O)N2CCCCC2c2ncc(-c3ccc(C(=O)O)cc3)cc2F)ncc1C1(C(F)(F)F)CC1]

To a solution of methyl 4-(5-fluoro-6-(1-((4-methyl-5-(1-(trifluoromethyl)cyclopropyl)pyridin-2-yl)carbamoyl)piperidin-2-yl)pyridin-3-yl)benzoate (70.0 mg, 0.120 mmol) in tetrahydrofuran (2.0 mL) and water (0.30 mL) at room temperature was added lithium hydroxide monohydrate (52.9 mg, 1.26 mmol), After 1 hour, the mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford 4-(5-fluoro-6-(1-((4-methyl-5-(1-(trifluoromethyl)cyclopropyl)pyridin-2-yl)carbamoyl)piperidin-2-yl)pyridin-3-yl)benzoic acid (40.0 mg, 59%) as a white solid. LCMS (ESI) [M+H]⁺: 553.1.

### Step 3: (S)-4-(5-fluoro-6-(1-((4-methyl-5-(1-(trifluoromethyl)cyclopropyl)pyridin-2-yl)carbamoyl)piperidin-2-yl)pyridin-3-yl)benzoic acid and (R)-4-(5-fluoro-6-(1-((4-methyl-5-(1-(trifluoromethyl)cyclopropyl)pyridin-2-yl)carbamoyl)piperidin-2-yl)pyridin-3-yl)benzoic acid

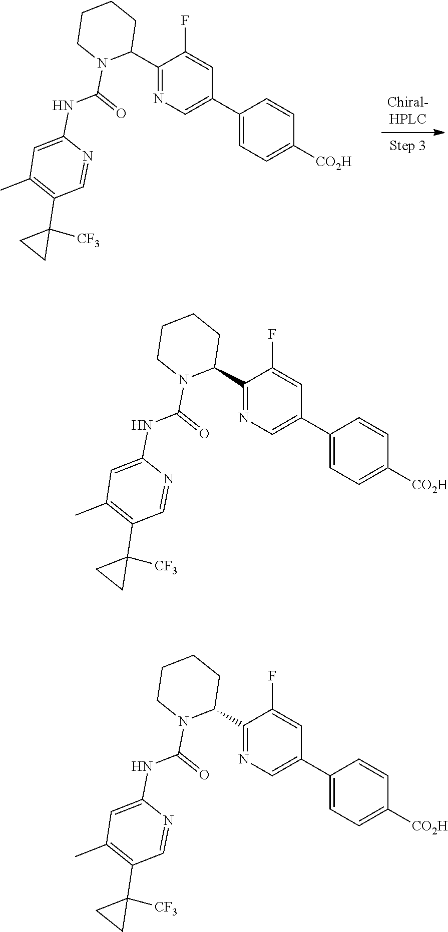 [SMILES: Cc1cc(NC(=O)N2CCCCC2c2ncc(-c3ccc(C(=O)O)cc3)cc2F)ncc1C1(C(F)(F)F)CC1 ; Cc1cc(NC(=O)N2CCCC[C@H]2c2ncc(-c3ccc(C(=O)O)cc3)cc2F)ncc1C1(C(F)(F)F)CC1 ; Cc1cc(NC(=O)N2CCCC[C@@H]2c2ncc(-c3ccc(C(=O)O)cc3)cc2F)ncc1C1(C(F)(F)F)CC1]

4-(5-fluoro-6-(1-((4-methyl-5-(1-(trifluoromethyl)cyclopropyl)pyridin-2-yl)carbamoyl)piperidin-2-yl)pyridin-3-yl)benzoic acid (40.0 mg) was resolved by preparative chiral HPLC (conditions: column: CHIRALPAK IK 3*25 cm, 5 μm; mobile phase A: hexane (0.1% formic acid), mobile phase B: ethanol; flow rate: 40 mL/min; gradient (B %): isocratic, 30%; detector: 220/254 nm; RT1 (min): 6.1; RT2 (min): 17.2) to afford (Compound 205a; Method G, Peak 1, 0.89 min) (S)-4-(5-fluoro-6-(1-((4-methyl-5-(1-(trifluoromethyl)cyclopropyl)pyridin-2-yl)carbamoyl)piperidin-2-yl)pyridin-3-yl)benzoic acid (1^{st} peak, 19.6 mg, 65%) as a white solid, and (Compound 205b; Method G, Peak 2, 1.98 min) (R)-4-(5-fluoro-6-(1-((4-methyl-5-(1-(trifluoromethyl)cyclopropyl)pyridin-2-yl)carbamoyl)piperidin-2-yl)pyridin-3-yl)benzoic acid (2^{nd} peak, 15.4 mg, 51%) as a white solid.

| Cmpd # | Characterization Data |
|---|---|
| 205a | LCMS (ESI) [M + H]⁺: 543.2 |
| | ¹H NMR (400 MHz, methanol-d₄) δ 8.69 (s, 1H), 8.17 (s, 1H), 8.15-8.07 (m, 2H), 7.88 (dd, J = |
| | 11.6, 2.0 Hz, 1H), 7.78 (d, J = 8.2 Hz, 2H), 7.62 (s, 1H), 5.86-5.77 (m, 1H), 4.09-4.00 (m, 1H), |
| | 3.88-3.76 (m, 1H), 2.41 (s, 3H), 2.37-2.27 (m, 1H), 2.11-1.99 (m, 1H), 1.90-1.82 (m, 1H), |
| | 1.75-1.64 (m, 2H), 1.62-1.52 (m, 1H), 1.47-1.41 (m, 2H), 1.10-1.04 (m, 2H). |
| 205b | LCMS (ESI) [M + H]⁺: 543.2 |
| | ¹H NMR (400 MHz, Methanol-d₄) δ 8.69 (s, 1H), 8.18 (s, 1H), 8.12 (d, J = 8.3 Hz, 2H), 7.88 (dd, |
| | J = 11.6, 1.9 Hz, 1H), 7.78 (d, J = 8.4 Hz, 2H), 7.62 (s, 1H), 5.86-5.77 (m, 1H), 4.10-4.01 (m, |
| | 1H), 3.88-3.74 (m, 1H), 2.41 (s, 3H), 2.36-2.27 (m, 1H), 2.10-1.98 (m, 1H), 1.91-1.79 (m, |
| | 1H), 1.76-1.62 (m, 2H), 1.61-1.52 (m, 1H), 1.47-1.42 (m, 2H), 1.09-1.02 (m, 2H). |

### Example 206: (S)-4-(5-fluoro-6-(4-((4-methyl-5-(1-(trifluoromethyl)cyclopropyl)pyridin-2-yl)carbamoyl)morpholin-3-yl)pyridin-3-yl)benzoic acid and (R)-4-(5-fluoro-6-(4-((4-methyl-5-(1-(trifluoromethyl)cyclopropyl)pyridin-2-yl)carbamoyl)morpholin-3-yl)pyridin-3-yl)benzoic acid

### Step 1: methyl 4-(5-fluoro-6-(4-((4-methyl-5-(1-(trifluoromethyl)cyclopropyl)pyridin-2-yl)carbamoyl)morpholin-3-yl)pyridin-3-yl)benzoate

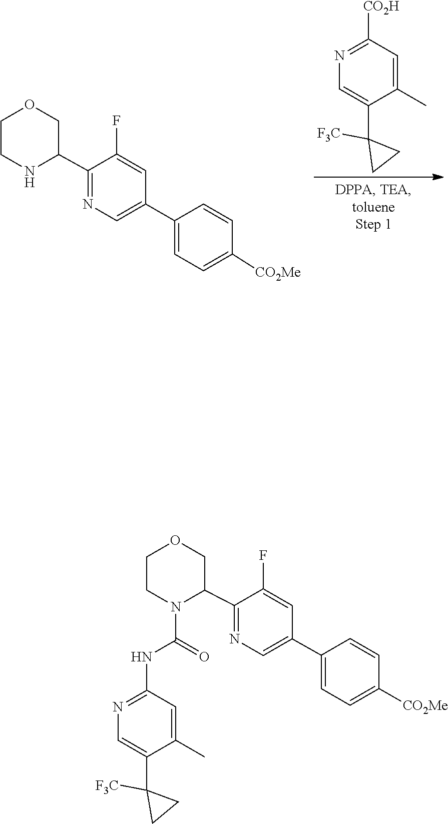 [SMILES: COC(=O)c1ccc(-c2cnc(C3COCCN3)c(F)c2)cc1 ; Cc1cc(C(=O)O)ncc1C1(C(F)(F)F)CC1 ; COC(=O)c1ccc(-c2cnc(C3COCCN3C(=O)Nc3cc(C)c(C4(C(F)(F)F)CC4)cn3)c(F)c2)cc1]

A solution of methyl 4-(5-fluoro-6-(morpholin-3-yl)pyridin-3-yl)benzoate (129 mg, 0.408 mmol), 4-methyl-5-(1-(trifluoromethyl)cyclopropyl)picolinic acid (100 mg, 0.408 mmol), diphenylphosphoryl azide (336 mg, 1.22 mmol) and triethylamine (123 mg, 1.22 mmol) in toluene (2.0 mL) was heated to 80° C. for 2 h. Upon completion, the mixture was concentrated under reduced pressure, and the crude residue was purified by silica gel column chromatography, eluted with petroleum ether/ethyl acetate (1:1), to afford methyl 4-(5-fluoro-6-(4-((4-methyl-5-(1-(trifluoromethyl)cyclopropyl)pyridin-2-yl)carbamoyl)morpholin-3-yl)pyridin-3-yl)benzoate (90 mg, 39%) as a white solid. LCMS (ESI) [M+H]⁺: 559.1.

### Step 2: 4-(5-fluoro-6-(4-((4-methyl-5-(1-(trifluoromethyl)cyclopropyl)pyridin-2-yl)carbamoyl)morpholin-3-yl)pyridin-3-yl)benzoic acid

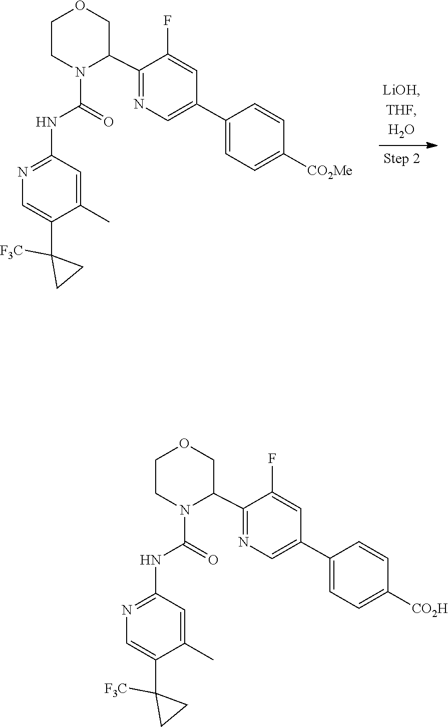 [SMILES: COC(=O)c1ccc(-c2cnc(C3COCCN3C(=O)Nc3cc(C)c(C4(C(F)(F)F)CC4)cn3)c(F)c2)cc1 ; Cc1cc(NC(=O)N2CCOCC2c2ncc(-c3ccc(C(=O)O)cc3)cc2F)ncc1C1(C(F)(F)F)CC1]

A solution of methyl 4-(5-fluoro-6-(4-((4-methyl-5-(1-(trifluoromethyl)cyclopropyl)pyridin-2-yl)carbamoyl)morpholin-3-yl)pyridin-3-yl)benzoate (90.0 mg, 0.161 mmol) and lithium hydroxide monohydrate (34.0 mg, 0.805 mmol) in tetrahydrofuran (2.0 mL) and water (0.30 mL) was stirred at room temperature for 1 hour. Upon completion, the mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford 4-(5-fluoro-6-(4-((4-methyl-5-(1-(trifluoromethyl)cyclopropyl)pyridin-2-yl)carbamoyl)morpholin-3-yl)pyridin-3-yl)benzoic acid (40.0 mg, 56%) as a white solid. LCMS (ESI) [M+H]⁺: 545.1.

### Step 3: (S)-4-(5-fluoro-6-(4-((4-methyl-5-(1-(trifluoromethyl)cyclopropyl)pyridin-2-yl)carbamoyl)morpholin-3-yl)pyridin-3-yl)benzoic acid and (R)-4-(5-fluoro-6-(4-((4-methyl-5-(1-(trifluoromethyl)cyclopropyl)pyridin-2-yl)carbamoyl)morpholin-3-yl)pyridin-3-yl)benzoic acid

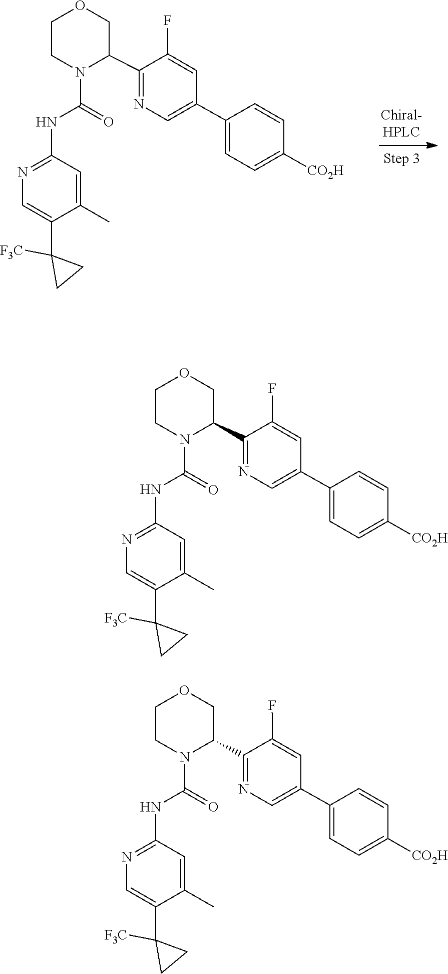

4-(5-fluoro-6-(4-((4-methyl-5-(1-(trifluoromethyl)cyclopropyl)pyridin-2-yl)carbamoyl)morpholin-3-yl)pyridin-3-yl)benzoic acid (40.0 mg) was resolved by preparative chiral HPLC (conditions: column: CHIRALPAK IA, 3*25 cm, 5 μm; mobile phase A: hexane/MTBE (1:1; 0.1% formic acid); mobile phase B: methanol; flow rate: 40 mL/min; gradient (B %): isocratic, 20%; detector: 220/254 nm; RT1 (min): 8.8; RT2 (min): 17.2) to afford (Compound 206a; Method BD, Peak 1, 1.11 min) (S)-4-(5-fluoro-6-(4-((4-methyl-5-(1-(trifluoromethyl)cyclopropyl)pyridin-2-yl)carbamoyl)morpholin-3-yl)pyridin-3-yl)benzoic acid (1^{st} peak, 10.4 mg, 26%) as a white solid, and (Compound 206b; Method BD, Peak 2, 1.81 min) (R)-4-(5-fluoro-6-(4-((4-methyl-5-(1-(trifluoromethyl)cyclopropyl)pyridin-2-yl)carbamoyl)morpholin-3-yl)pyridin-3-yl)benzoic acid (2^{nd} peak, 10.1 mg, 25%) as a white solid.

| Cmpd # | Characterization Data |
|---|---|
| 206a | LCMS (ESI) [M + H]⁺: 545.2 |
| | ¹H NMR (400 MHz, Methanol-d₄) δ 8.70 (t, J = 1.6 Hz, 1H), 8.20 (s, 1H), 8.17-8.09 (m, 2H), 7.93 |
| | (dd, J = 11.6, 1.9 Hz, 1H), 7.80 (d, J = 8.5 Hz, 2H), 7.60 (s, 1H), 5.58-5.52 (m, 1H), 4.44 (d, J = |
| | 12.1 Hz, 1H), 4.14-3.98 (m, 3H), 3.95-3.88 (m, 1H), 3.77-3.66 (m, 1H), 2.46 (s, 3H), 1.52- |
| | 1.42 (m, 2H), 1.14-1.05 (m, 2H). |
| 206b | LCMS (ESI) [M + H]⁺: 545.2 |
| | ¹H NMR (400 MHz, Methanol-d₄) δ 8.70 (t, J = 1.7 Hz, 1H), 8.20 (s, 1H), 8.16-8.10 (m, 2H), 7.94 |
| | (dd, J = 11.6, 1.9 Hz, 1H), 7.80 (d, J = 8.4 Hz, 2H), 7.59 (s, 1H), 5.59-5.53 (m, 1H), 4.45 (d, J = |
| | 12.1 Hz, 1H), 4.15-3.96 (m, 3H), 3.95-3.86 (m, 1H), 3.78-3.65 (m, 1H), 2.48 (s, 3H), 1.52- |
| | 1.43 (m, 2H), 1.14-1.04 (m, 2H). |

### Example 207: (R)-4-(5-fluoro-6-(4-((5-fluoro-6-(1-(trifluoromethyl)cyclopropyl)pyridin-3-yl)carbamoyl)morpholin-3-yl)pyridin-3-yl)benzoic acid and (S)-4-(5-fluoro-6-(4-((5-fluoro-6-(1-(trifluoromethyl)cyclopropyl)pyridin-3-yl)carbamoyl)morpholin-3-yl)pyridin-3-yl)benzoic acid

### Step 1: methyl 4-(5-fluoro-6-(4-((5-fluoro-6-(1-(trifluoromethyl)cyclopropyl)pyridin-3-yl)carbamoyl)morpholin-3-yl)pyridin-3-yl)benzoate

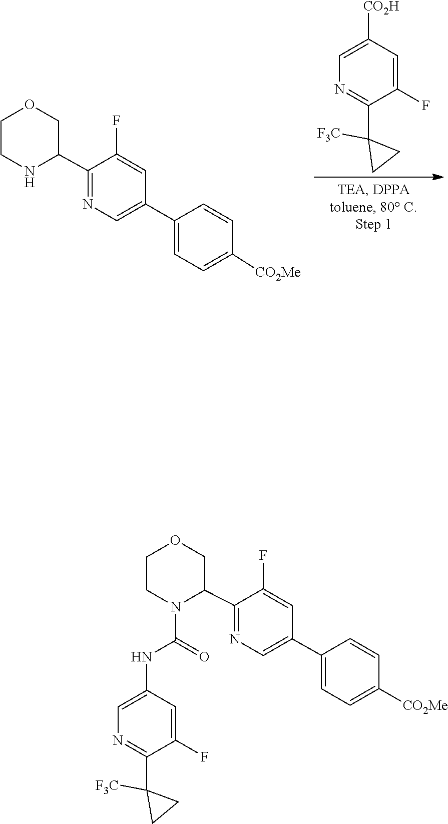 [SMILES: COC(=O)c1ccc(-c2cnc(C3COCCN3)c(F)c2)cc1 ; O=C(O)c1cnc(C2(C(F)(F)F)CC2)c(F)c1 ; COC(=O)c1ccc(-c2cnc(C3COCCN3C(=O)Nc3cnc(C4(C(F)(F)F)CC4)c(F)c3)c(F)c2)cc1]

To a solution of methyl 4-(5-fluoro-6-(morpholin-3-yl)pyridin-3-yl)benzoate (64.0 mg, 0.201 mmol) in toluene (2.0 mL) was added 5-fluoro-6-(1-(trifluoromethyl)cyclopropyl)nicotinic acid (50 mg, 0.201 mmol), diphenylphosphoryl azide (110 mg, 0.402 mmol), and triethylamine (61.0 mg, 0.603 mmol). The resultant mixture was heated at 80° C. for 2 hours. Upon completion, the mixture was cooled to room temperature and concentrated under reduced pressure. The crude residue was directly purified by silica gel column chromatography, eluted with petroleum ether/ethyl acetate (1:1), to afford methyl 4-(5-fluoro-6-(4-((5-fluoro-6-(1-(trifluoromethyl)cyclopropyl)pyridin-3-yl)carbamoyl)morpholin-3-yl)pyridin-3-yl)benzoate (40 mg, 35%) as a white solid. LCMS (ESI) [M+H]⁺: 563.1.

### Step 2: 4-(5-fluoro-6-(4-((5-fluoro-6-(1-(trifluoromethyl)cyclopropyl)pyridin-3-yl)carbamoyl)morpholin-3-yl)pyridin-3-yl)benzoic acid

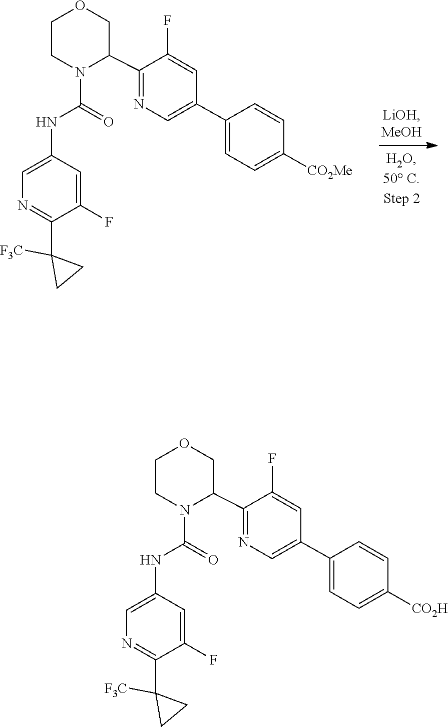 [SMILES: COC(=O)c1ccc(-c2cnc(C3COCCN3C(=O)Nc3cnc(C4(C(F)(F)F)CC4)c(F)c3)c(F)c2)cc1 ; O=C(O)c1ccc(-c2cnc(C3COCCN3C(=O)Nc3cnc(C4(C(F)(F)F)CC4)c(F)c3)c(F)c2)cc1]

To a solution of methyl 4-(5-fluoro-6-(4-((5-fluoro-6-(1-(trifluoromethyl)cyclopropyl)pyridin-3-yl)carbamoyl)morpholin-3-yl)pyridin-3-yl)benzoate (40 mg, 0.071 mmol) in methanol (2.0 mL) and water (0.30 mL) as added lithium hydroxide monohydrate (15 mg, 0.355 mmol). The resultant mixture was heated to 50° C. for 1 hour. Upon completion, the mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford 4-(5-fluoro-6-(4-((5-fluoro-6-(1-(trifluoromethyl)cyclopropyl)pyridin-3-yl)carbamoyl)morpholin-3-yl)pyridin-3-yl)benzoic acid (30.0 mg, 77%) as a white solid. LCMS (ESI) [M+H]⁺: 549.1.

### Step 3: (R)-4-(5-fluoro-6-(4-((5-fluoro-6-(1-(trifluoromethyl)cyclopropyl)pyridin-3-yl)carbamoyl)morpholin-3-yl)pyridin-3-yl)benzoic acid and (S)-4-(5-fluoro-6-(4-((5-fluoro-6-(1-(trifluoromethyl)cyclopropyl)pyridin-3-yl)carbamoyl)morpholin-3-yl)pyridin-3-yl)benzoic acid

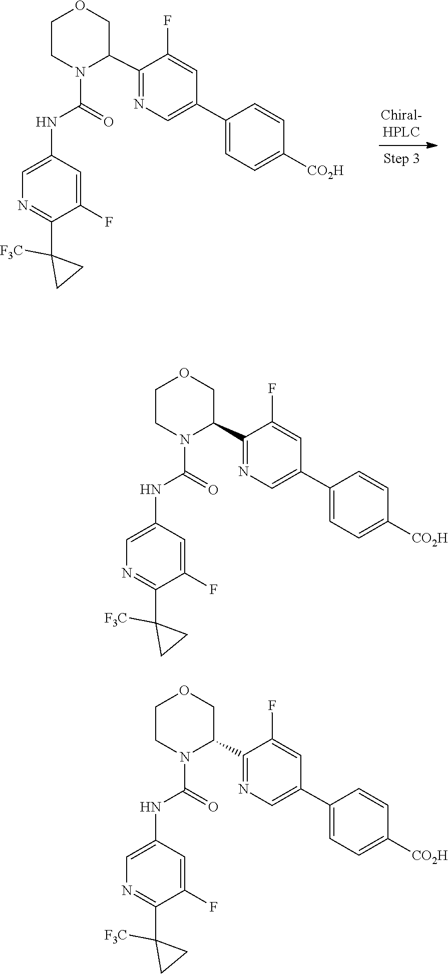

4-(5-fluoro-6-(4-((5-fluoro-6-(1-(trifluoromethyl)cyclopropyl)pyridin-3-yl)carbamoyl)morpholin-3-yl)pyridin-3-yl)benzoic acid (40.0 mg) was resolved by preparative chiral HPLC (conditions: column: CHIRAL ART Cellulose-SZ, 3*25 cm, 5 μm; mobile phase A: hexane (0.1% formic acid), mobile phase B: ethanol; flow rate: 40 mL/min; gradient (B %): isocratic, 50% B; detector: 220/254 nm; RT1 (min): 6.1; RT2 (min): 7.8) to afford (Compound 207a; Method BE, Peak 1, 0.68 min) (R)-4-(5-fluoro-6-(4-((5-fluoro-6-(1-(trifluoromethyl)cyclopropyl)pyridin-3-yl)carbamoyl)morpholin-3-yl)pyridin-3-yl)benzoic acid (1^{st} peak, 13.8 mg, 46%) as a white solid and (Compound 207b; Method BE, Peak 2, 0.94 min) (S)-4-(5-fluoro-6-(4-((5-fluoro-6-(1-(trifluoromethyl)cyclopropyl)pyridin-3-yl)carbamoyl)morpholin-3-yl)pyridin-3-yl)benzoic acid (2^{nd} peak, 13.7 mg, 45%) as a white solid.

| Cmpd # | Characterization Data |
|---|---|
| 207a | LCMS (ESI) [M + H]⁺: 549.2 |
| | ¹H NMR (400 MHz, DMSO-d₆) δ 9.21 (s, 1H), 8.77 (s, 1H), 8.49 (s, 1H), 8.15 (dd, J = 12.0, 1.9 |
| | Hz, 1H), 8.11-8.00 (m, 2H), 7.95-7.81 (m, 3H), 5.57-5.40 (m, 1H), 4.30 (d, J = 11.9 Hz, 1H), |
| | 4.05-3.81 (m, 4H), 3.60 (td, J = 11.0, 4.1 Hz, 1H), 1.42-1.32 (m, 2H), 1.22-1.08 (m, 2H). |
| 207b | LCMS (ESI) [M + H]⁺: 549.2 |
| | ¹H NMR (400 MHz, DMSO-d₆) δ 9.21 (s, 1H), 8.77 (s, 1H), 8.49 (s, 1H), 8.16 (dd, J = 12.0, 1.9 |
| | Hz, 1H), 8.04 (d, J = 8.4 Hz, 2H), 7.96-7.85 (m, 3H), 5.53-5.45 (m, 1H), 4.30 (d, J = 12.0 Hz, |
| | 1H), 4.04-3.85 (m, 4H), 3.61 (dt, J = 11.0, 5.5 Hz, 1H), 1.42-1.34 (m, 2H), 1.23-1.11 (m, |
| | 2H). |

### Example 208: (R)-2-(3-fluoro-5-(4-((trifluoromethyl)sulfonamido)phenyl)pyridin-2-yl)-N-(4-isopropylphenyl)pyrrolidine-1-carboxamide

### Step 1: 1,1,1-trifluoro-N-(4-(4,4,5,5-tetramethyl-1,3,2-dioxaborolan-2-yl)phenyl)methanesulfonamide

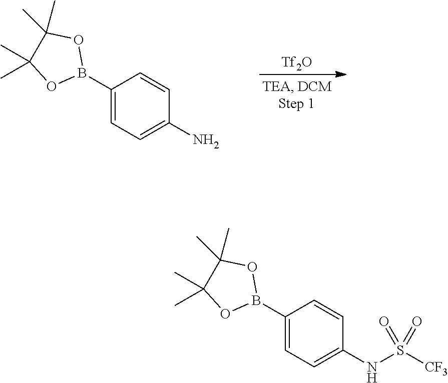 [SMILES: "Tf2O" ; CC1(C)OB(c2ccc(N)cc2)OC1(C)C ; CC1(C)OB(c2ccc(NS(=O)(=O)C(F)(F)F)cc2)OC1(C)C]

To a solution of 4-(4,4,5,5-tetramethyl-1,3,2-dioxaborolan-2-yl)aniline (200 mg, 0.913 mmol) and triethylamine (277 mg, 2.74 mmol) in dichloromethane (4.0 mL) at 0° C. was added and trifluoromethanesulfonic anhydride (283 mg, 1.00 mmol). After 2 hours, the mixture was concentrated under reduced pressure. The crude residue was directly purified by silica gel column chromatography, eluted with petroleum ether/ethyl acetate (1:1) to afford 1,1,1-trifluoro-N-(4-(4,4,5,5-tetramethyl-1,3,2-dioxaborolan-2-yl)phenyl)methanesulfonamide (130 mg, 40%) as a yellow solid. LCMS (ESI) [M+H]⁺: 352.0.

### Step 2: (R)-2-(3-fluoro-5-(4-((trifluoromethyl)sulfonamido)phenyl)pyridin-2-yl)-N-(4-isopropylphenyl)pyrrolidine-1-carboxamide

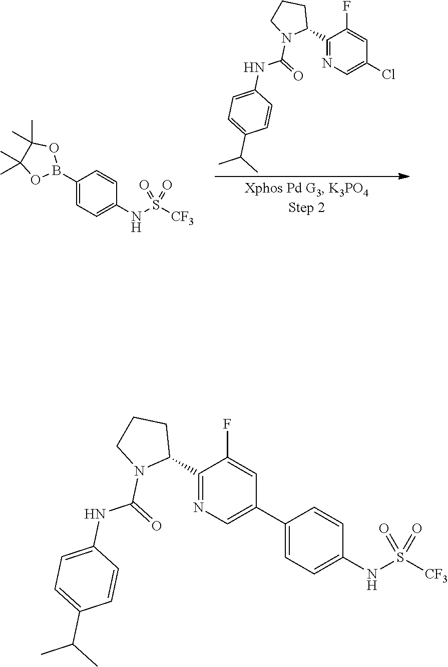 [SMILES: CC1(C)OB(c2ccc(NS(=O)(=O)C(F)(F)F)cc2)OC1(C)C ; CC(C)c1ccc(NC(=O)N2CCC[C@@H]2c2ncc(Cl)cc2F)cc1 ; CC(C)c1ccc(NC(=O)N2CCC[C@@H]2c2ncc(-c3ccc(NS(=O)(=O)C(F)(F)F)cc3)cc2F)cc1]

To a solution of (R)-2-(5-chloro-3-fluoropyridin-2-yl)-N-(4-isopropylphenyl)pyrrolidine-1-carboxamide (133 mg, 0.370 mmol), 1,1,1-trifluoro-N-(4-(4,4,5,5-tetramethyl-1,3,2-dioxaborolan-2-yl)phenyl)methanesulfonamide (130 mg, 0.370 mmol), and tribasic potassium phosphate (235 mg, 1.11 mmol) in 1,4-dioxane (1.5 mL) and water (0.50 mL) was added XPhos Pd G3 (31 mg, 0.037 mmol). The resultant mixture was heated to 90° C. for 1 hour. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 40 min; detector: UV 254 nm) to afford (Compound 208) (R)-2-(3-fluoro-5-(4-((trifluoromethyl)sulfonamido)phenyl)pyridin-2-yl)-N-(4-isopropylphenyl)pyrrolidine-1-carboxamide (25 mg, 12%) as a white solid.

| Cmpd # | Characterization Data |
|---|---|
| 208 | LCMS (ESI) [M + H]⁺: 551.1 |
| | ¹H NMR (400 MHz, methanol-d₄) δ 8.57 (s, 1H), 7.82 (dd, J = 11.3, 1.9 Hz, 1H), 7.69 (d, J = 8.6 |
| | Hz, 2H), 7.40 (d, J = 8.6 Hz, 2H), 7.24 (d, J = 8.5 Hz, 2H), 7.09 (d, J = 8.5 Hz, 2H), 5.47-5.40 |
| | (m, 1H), 3.90-3.81 (m, 1H), 3.73-3.63 (m, 1H), 2.81 (hept, J = 6.9 Hz, 1H), 2.50-2.38 (m, |
| | 1H), 2.23-2.15 (m, 1H), 2.15-2.05 (m, 1H), 2.05-1.96 (m, 1H), 1.19 (d, J = 6.9 Hz, 6H). |

### Example 209: (R)-2-(3-fluoro-5-(4-oxo-3,4-dihydrobenzo[d][1,2,3]triazin-7-yl)pyridin-2-yl)-N-(4-isopropylphenyl)pyrrolidine-1-carboxamide

### Step 1: 7-bromobenzo[d][1,2,3]triazin-4(3H)-one

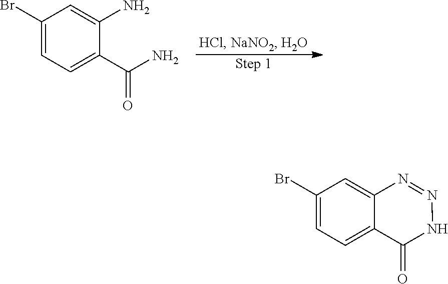

To a solution of 2-amino-4-bromobenzamide (500 mg, 2.32 mmol) and sodium nitrite (320 mg, 4.65 mmol) in water (4.0 mL) at 0° C. was added concentrated hydrochloric acid (3.0 mL) dropwise. The resultant mixture was warmed room temperature and stirred for 2 hours. Upon completion, the reaction solution was adjusted to pH ˜6.0 with saturated sodium carbonate and extracted with ethyl acetate (2×5 mL). The combined organic layers were concentrated under reduced pressure. The crude residue was directly purified by silica gel column chromatography, eluted with petroleum ether/ethyl acetate (1:1), to afford 7-bromobenzo[d][1,2,3]triazin-4(3H)-one (200 mg, 38%) as a white solid. LCMS (ESI) [M+H]⁺: 226.0.

### Step 2: 7-bromo-3-((2-(trimethylsilyl)ethoxy)methyl)benzo[d][1,2,3]triazin-4(3H)-one

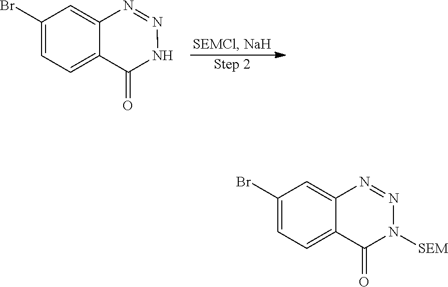

To a solution of 7-bromobenzo[d][1,2,3]triazin-4(3H)-one (200 mg, 0.881 mmol) in tetrahydrofuran (5.0 mL) at 0° C. was added sodium hydride (60% in mineral oil, 27 mg, 1.15 mmol). After 30 minutes, [2-(chloromethoxy)ethyl]trimethylsilane (147 mg, 0.884 mmol) was added dropwise. The mixture was warmed to room temperature and stirred for and additional 1 hour. Upon completion, the mixture was concentrated under reduced pressure. The crude residue was purified by silica gel column chromatography, eluted with petroleum ether/ethyl acetate (1:1), to afford 7-bromo-3-((2-(trimethylsilyl)ethoxy)methyl)benzo[d][1,2,3]triazin-4(3H)-one (110 mg, 34%) as a white solid. LCMS (ESI) [M+H]⁺: 356.0.

### Step 3: (R)-2-(3-fluoro-5-(4-oxo-3-((2-(trimethylsilyl)ethoxy)methyl)-3,4-dihydrobenzo[d][1,2,3]triazin-7-yl)pyridin-2-yl)-N-(4-isopropylphenyl)pyrrolidine-1-carboxamide

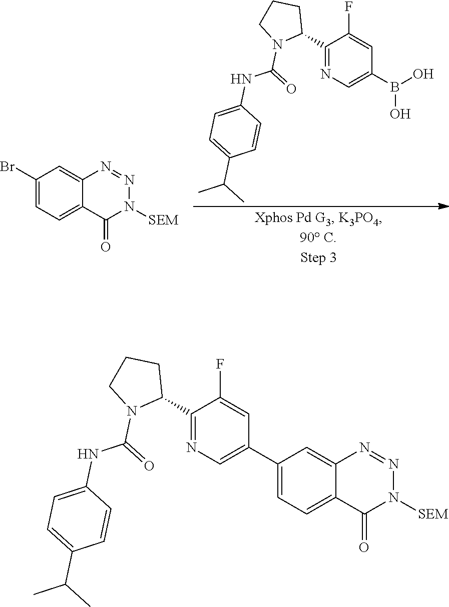 [SMILES: C[Si](C)(C)CCOCn1nnc2cc(Br)ccc2c1=O ; CC(C)c1ccc(NC(=O)N2CCC[C@@H]2c2ncc(B(O)O)cc2F)cc1 ; CC(C)c1ccc(NC(=O)N2CCC[C@@H]2c2ncc(-c3ccc4c(=O)n(COCC[Si](C)(C)C)nnc4c3)cc2F)cc1]

To a solution of 7-bromo-3-((2-(trimethylsilyl)ethoxy)methyl)benzo[d][1,2,3]triazin-4(3H)-one (110 mg, 0.300 mmol), (R)-(5-fluoro-6-(1-((4-isopropylphenyl)carbamoyl)pyrrolidin-2-yl)pyridin-3-yl)boronic acid (115 mg, 0.303 mmol), and tribasic potassium phosphate (196 mg, 0.926 mmol) in tetrahydrofuran (4.0 mL) and water (1.0 mL) was added XPhos Pd G3 (26 mg, 0.031 mmol). The resultant mixture was heated to 90° C. for 1.0 hour. Upon completion, the mixture was concentrated under reduced pressure. The crude residue was purified by silica gel column chromatography, eluted with petroleum ether/ethyl acetate (1:1), to afford (R)-2-(3-fluoro-5-(4-oxo-3-((2-(trimethylsilyl)ethoxy)methyl)-3,4-dihydrobenzo[d][1,2,3]triazin-7-yl)pyridin-2-yl)-N-(4-isopropylphenyl)pyrrolidine-1-carboxamide (70.0 mg, 37%) as a white solid. LCMS (ESI) [M+H]⁺: 603.2.

### Step 4: (R)-2-(3-fluoro-5-(4-oxo-3,4-dihydrobenzo[d][1,2,3]triazin-7-yl)pyridin-2-yl)-N-(4-isopropylphenyl)pyrrolidine-1-carboxamide

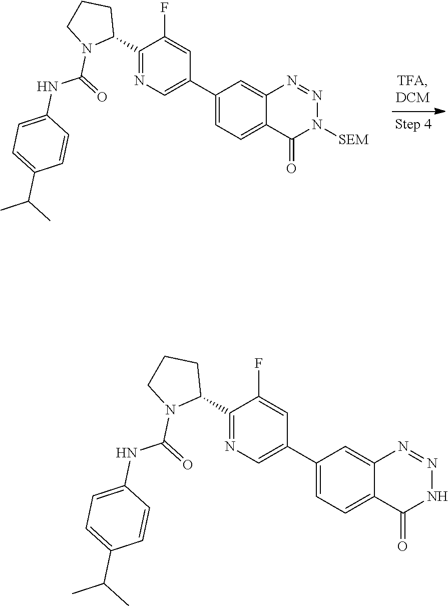 [SMILES: CC(C)c1ccc(NC(=O)N2CCC[C@@H]2c2ncc(-c3ccc4c(=O)n(COCC[Si](C)(C)C)nnc4c3)cc2F)cc1 ; CC(C)c1ccc(NC(=O)N2CCC[C@@H]2c2ncc(-c3ccc4c(=O)[nH]nnc4c3)cc2F)cc1]

To a solution of (R)-2-(3-fluoro-5-(4-oxo-3-((2-(trimethylsilyl)ethoxy)methyl)-3,4-dihydrobenzo[d][1,2,3]triazin-7-yl)pyridin-2-yl)-N-(4-isopropylphenyl)pyrrolidine-1-carboxamide (70.0 mg, 0.114 mmol) in dichloromethane (2.5 mL) at room temperature was added trifluoroacetic acid (2.5 mL). After 3 hours, the mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 60% over 20 min; detector: UV 254 nm) to afford (Compound 209) (R)-2-(3-fluoro-5-(4-oxo-3,4-dihydrobenzo[d][1,2,3]triazin-7-yl)pyridin-2-yl)-N-(4-isopropylphenyl)pyrrolidine-1-carboxamide (10.6 mg, 19%) as a white solid.

| Cmpd # | Characterization Data |
|---|---|
| 209 | LCMS (ESI) [M + H]⁺: 473.3 |
| | ¹H NMR (400 MHz, Methanol-d₄) δ 8.78 (s, 1H), 8.47 (d, J = 1.8 Hz, 1H), 8.39 (d, J = 8.3 Hz, 1H), |
| | 8.20 (dd, J = 8.3, 1.8 Hz, 1H), 8.06 (dd, J = 11.0, 1.9 Hz, 1H), 7.24 (d, J = 8.5 Hz, 2H), 7.09 (d, J = |
| | 8.6 Hz, 2H), 5.53-5.41 (m, 1H), 3.92-3.82 (m, 1H), 3.76-3.66 (m, 1H), 2.82 (hept, J = 7.0 Hz, |
| | 1H), 2.52-2.40 (m, 1H), 2.27-2.16 (m, 1H), 2.16-2.10 (m, 1H), 2.07-2.00 (m, 1H), 1.19 (d, J = |
| | 6.9 Hz, 6H). |

### Example 210: (R)-2-(3-fluoro-5-(4-oxo-3,4-dihydrobenzo[d][1,2,3]triazin-6-yl)pyridin-2-yl)-N-(4-isopropylphenyl)pyrrolidine-1-carboxamide

### Step 1: 6-bromobenzo[d][1,2,3]triazin-4(3H)-one

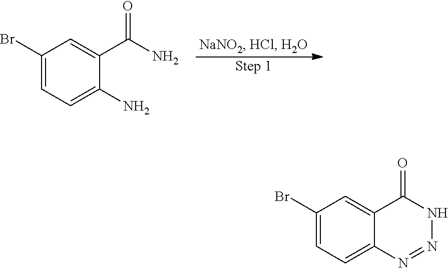

To a solution of 2-amino-5-bromobenzamide (500 mg, 2.32 mmol) and sodium nitrite (320 mg, 4.65 mmol) in water (4.0 mL) at 0° C. was added concentrated hydrochloric acid (3.0 mL) dropwise. The resultant mixture was warmed to room temperature and stirred for 2 hours. Upon completion, the reaction solution was adjusted to pH ˜6.0 with saturated sodium carbonate solution and extracted with ethyl acetated (2×5 mL). The combined organic layers were concentrated under reduced pressure. The crude residue was purified by silica gel column chromatography, eluted with petroleum ether/ethyl acetate (1:1), to afford 6-bromobenzo[d][1,2,3]triazin-4(3H)-one (204 mg, 39%) as a white solid. LCMS (ESI) [M+H]⁺: 226.0.

### Step 2: 6-bromo-3-((2-(trimethylsilyl)ethoxy)methyl)benzo[d][1,2,3]triazin-4(3H)-one

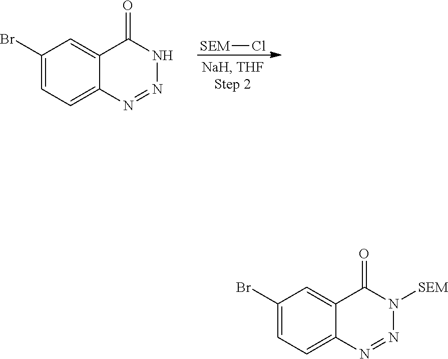 [SMILES: O=c1[nH]nnc2ccc(Br)cc12 ; C[Si](C)(C)CCOCCl ; C[Si](C)(C)CCOCn1nnc2ccc(Br)cc2c1=O]

To a solution of 6-bromobenzo[d][1,2,3]triazin-4(3H)-one (200 mg, 0.881 mmol) in tetrahydrofuran (5.0 mL) at 0° C. was added sodium hydride (60% in mineral oil, 27 mg, 1.15 mmol). After 30 minutes, [2-(chloromethoxy)ethyl]trimethylsilane (147 mg, 0.884 mmol) was added dropwise. The reaction was warmed to room temperature and stirred for 1 hour. Upon completion, the mixture was concentrated under reduced pressure. The crude residue was directly purified by silica gel column chromatography, eluted with petroleum ether/ethyl acetate (1:1), to afford 6-bromo-3-((2-(trimethylsilyl)ethoxy)methyl)benzo[d][1,2,3]triazin-4(3H)-one (118 mg, 37%) as a white solid. LCMS (ESI) [M+H]⁺: 356.0.

### Step 3: (R)-2-(3-fluoro-5-(4-oxo-3-((2-(trimethylsilyl)ethoxy)methyl)-3,4-dihydrobenzo[d][1,2,3]triazin-6-yl)pyridin-2-yl)-N-(4-isopropylphenyl)pyrrolidine-1-carboxamide

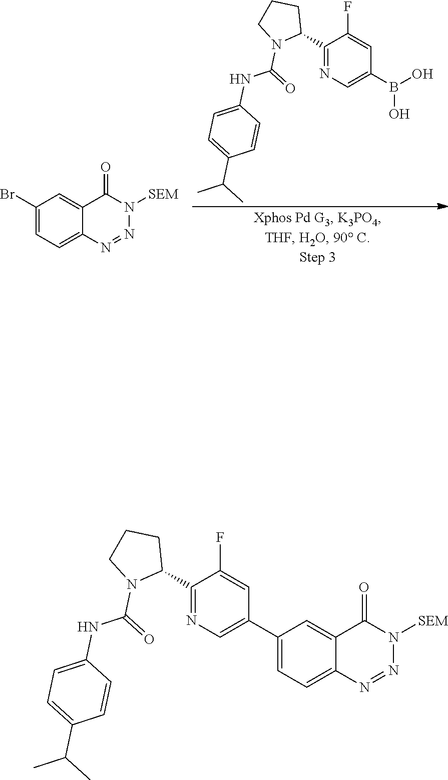 [SMILES: C[Si](C)(C)CCOCn1nnc2ccc(Br)cc2c1=O ; CC(C)c1ccc(NC(=O)N2CCC[C@@H]2c2ncc(B(O)O)cc2F)cc1 ; CC(C)c1ccc(NC(=O)N2CCC[C@@H]2c2ncc(-c3ccc4nnn(COCC[Si](C)(C)C)c(=O)c4c3)cc2F)cc1]

To a solution of 6-bromo-3-((2-(trimethylsilyl)ethoxy)methyl)benzo[d][1,2,3]triazin-4(3H)-one (110 mg, 0.300 mmol), (R)-(5-fluoro-6-(1-((4-isopropylphenyl)carbamoyl)pyrrolidin-2-yl)pyridin-3-yl)boronic acid (115 mg, 0.303 mmol), and tribasic potassium phosphate (196 mg, 0.926 mmol) in tetrahydrofuran (4.0 mL) and water (1.0 mL) was added XPhos Pd G3 (26 mg, 0.031 mmol). The resultant mixture was heated to 90° C. for 1.0 hour. Upon completion, the mixture was concentrated under reduced pressure. The crude residue was directly purified by silica gel column chromatography, eluted with petroleum ether/ethyl acetate (1:1) to afford (R)-2-(3-fluoro-5-(4-oxo-3-((2-(trimethylsilyl)ethoxy)methyl)-3,4-dihydrobenzo[d][1,2,3]triazin-6-yl)pyridin-2-yl)-N-(4-isopropylphenyl)pyrrolidine-1-carboxamide (80.0 mg, 43%) as a white solid. LCMS (ESI) [M+H]⁺: 603.2.

### Step 4: (R)-2-(3-fluoro-5-(4-oxo-3,4-dihydrobenzo[d][1,2,3]triazin-6-yl)pyridin-2-yl)-N-(4-isopropylphenyl)pyrrolidine-1-carboxamide

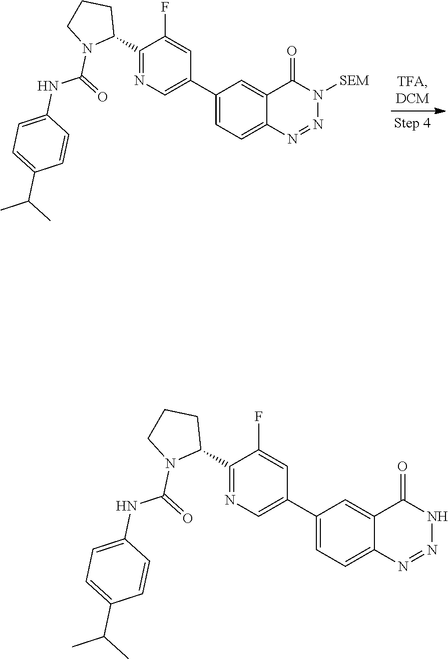 [SMILES: CC(C)c1ccc(NC(=O)N2CCC[C@@H]2c2ncc(-c3ccc4nnn(COCC[Si](C)(C)C)c(=O)c4c3)cc2F)cc1 ; CC(C)c1ccc(NC(=O)N2CCC[C@@H]2c2ncc(-c3ccc4nn[nH]c(=O)c4c3)cc2F)cc1]

To a solution of (R)-2-(3-fluoro-5-(4-oxo-3-((2-(trimethylsilyl)ethoxy)methyl)-3,4-dihydrobenzo[d][1,2,3]triazin-6-yl)pyridin-2-yl)-N-(4-isopropylphenyl)pyrrolidine-1-carboxamide (80.0 mg, 0.133 mmol) in dichloromethane (2.5 mL) at room temperature was added trifluoroacetic acid (2.5 mL). After 3 hours, the mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 60% over 20 min; detector: UV 254 nm) to afford (Compound 210) (R)-2-(3-fluoro-5-(4-oxo-3,4-dihydrobenzo[d][1,2,3]triazin-6-yl)pyridin-2-yl)-N-(4-isopropylphenyl)pyrrolidine-1-carboxamide (10.3 mg, 16%) as a white solid.

| Cmpd # | Characterization Data |
|---|---|
| 210 | LCMS (ESI) [M + H]⁺: 473.2 |
| | ¹H NMR (400 MHz, methanol-d₄) δ 8.76-8.71 (m, 1H), 8.55 (d, J = 2.0 Hz, 1H), 8.36 (dd, J = |
| | 8.5, 2.1 Hz, 1H), 8.25 (d, J = 8.5 Hz, 1H), 8.01 (dd, J = 11.1, 1.9 Hz, 1H), 7.28-7.20 (m, 2H), |
| | 7.12-7.04 (m, 2H), 5.52-5.43 (m, 1H), 3.91-3.81 (m, 1H), 3.76-3.64 (m, 1H), 2.81 (hept, J = |
| | 6.9 Hz, 1H), 2.54-2.40 (m, 1H), 2.27-1.98 (m, 3H), 1.18 (d, J = 6.9 Hz, 6H). |

### Example 211: (R)-2-(5-(3H-[1,2,3]triazolo[4,5-c]pyridin-6-yl)-3-fluoropyridin-2-yl)-N-(4-isopropylphenyl)pyrrolidine-1-carboxamide

### Step 1: 6-bromo-3H-[1,2,3]triazolo[4,5-c]pyridine

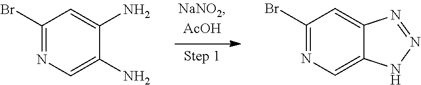 [SMILES: Nc1cnc(Br)cc1N ; Brc1cc2nn[nH]c2cn1]

To a stirred solution of sodium nitrite (11.0 mL, 22 mmol, 2.0 M in water) at 0° C. was added 6-bromopyridine-3,4-diamine (1.00 g, 5.31 mmol) followed by the slow addition of acetic acid (10.0 mL). The resultant mixture was heated to 70° C. for 2 hours. Upon completion, the mixture was poured into ice water (50 mL). The precipitated solids were collected by filtration, washed with water (3×3 mL) and dried under reduced pressure to afford 6-bromo-3H-[1,2,3]triazolo[4,5-c]pyridine (600 mg, 56%) as an off-white solid. LCMS (ESI) [M−H]: 197.0.

### Step 2: 6-bromo-3-((2-(trimethylsilyl)ethoxy)methyl)-3H-[1,2,3]triazolo[4,5-c]pyridine

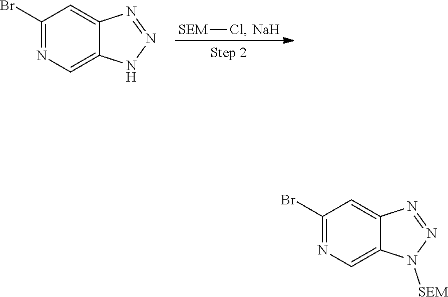

To a solution of 6-bromo-3H-[1,2,3]triazolo[4,5-c]pyridine (600 mg, 3.01 mmol) in tetrahydrofuran (10.0 mL) at 0° C. was added sodium hydride (60% in mineral oil, 216 mg, 9.04 mmol). After 30 minutes, [2-(chloromethoxy)ethyl]trimethylsilane (754 mg, 4.52 mmol) was added dropwise. The mixture was warmed to room temperature and stirred for 1.0 hour. Upon completion, the mixture was concentrated under reduced pressure. The crude residue was directly purified by silica gel column chromatography, eluted with petroleum ether/ethyl acetate (1:1) to afford 6-bromo-3-((2-(trimethylsilyl)ethoxy)methyl)-3H-[1,2,3]triazolo[4,5-c]pyridine (400 mg, 40%) as a yellow oil. LCMS (ESI) [M+H]⁺: 329.0.

### Step 3: (R)-2-(3-fluoro-5-(3-((2-(trimethylsilyl)ethoxy)methyl)-3H-[1,2,3]triazolo[4,5-c]pyridin-6-yl)pyridin-2-yl)-N-(4-isopropylphenyl)pyrrolidine-1-carboxamide

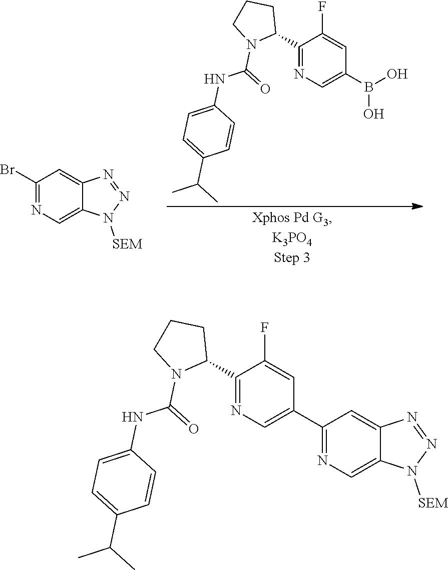 [SMILES: C[Si](C)(C)CCOCn1nnc2cc(Br)ncc21 ; CC(C)c1ccc(NC(=O)N2CCC[C@@H]2c2ncc(B(O)O)cc2F)cc1 ; CC(C)c1ccc(NC(=O)N2CCC[C@@H]2c2ncc(-c3cc4nnn(COCC[Si](C)(C)C)c4cn3)cc2F)cc1]

To a solution of 6-bromo-3-((2-(trimethylsilyl)ethoxy)methyl)-3H-[1,2,3]triazolo[4,5-c]pyridine (48.7 mg, 0.140 mmol), (R)-(5-fluoro-6-(1-((4-isopropylphenyl)carbamoyl)pyrrolidin-2-yl)pyridin-3-yl)boronic acid (54.9 mg, 0.140 mmol), and tribasic potassium phosphate (85.7 mg, 0.400 mmol) in tetrahydrofuran (4.0 mL) and water (1.0 mL) was added XPhos Pd G3 (11.4 mg, 0.010 mmol). The resultant mixture was heated to 90° C. for 1.0 hour. Upon completion, the mixture was concentrated under reduced pressure. The crude residue was directly purified by silica gel column chromatography, eluted with petroleum ether/ethyl acetate (1:1), to afford (R)-2-(3-fluoro-5-(3-((2-(trimethylsilyl)ethoxy)methyl)-3H-[1,2,3]triazolo[4,5-c]pyridin-6-yl)pyridin-2-yl)-N-(4-isopropylphenyl)pyrrolidine-1-carboxamide (60 mg, 77%) as a white solid. LCMS (ESI) [M+H]⁺: 576.3.

### Step 4: (R)-2-(5-(3H-[1,2,3]triazolo[4,5-c]pyridin-6-yl)-3-fluoropyridin-2-yl)-N-(4-isopropylphenyl)pyrrolidine-1-carboxamide

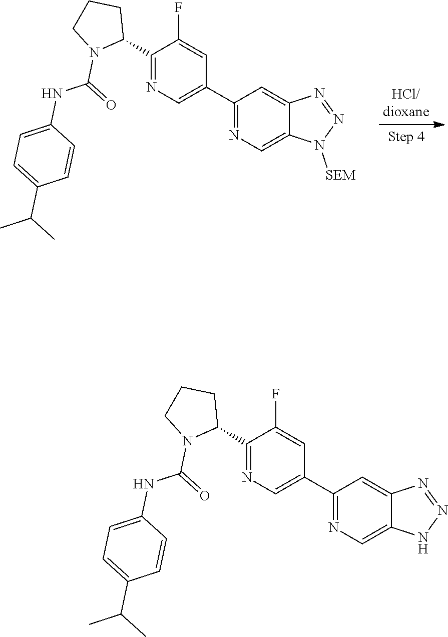 [SMILES: CC(C)c1ccc(NC(=O)N2CCC[C@@H]2c2ncc(-c3cc4nnn(COCC[Si](C)(C)C)c4cn3)cc2F)cc1 ; CC(C)c1ccc(NC(=O)N2CCC[C@@H]2c2ncc(-c3cc4nn[nH]c4cn3)cc2F)cc1]

To (R)-2-(3-fluoro-5-(3-((2-(trimethylsilyl)ethoxy)methyl)-3H-[1,2,3]triazolo[4,5-c]pyridin-6-yl)pyridin-2-yl)-N-(4-isopropylphenyl)pyrrolidine-1-carboxamide (60.0 mg, 0.100 mmol) in 1,4-dioxane (2.5 mL) at room temperature was added a solution of hydrochloric acid (1.30 mL, 5.20 mmol, 4.0 M in 1,4-dioxane). After 3 hours, the mixture was concentrated under reduced pressure. The crude residue was directly purified by preparative HPLC (conditions: column: XSelect CSH Fluoro Phenyl 30*150 mm, 5 μm; mobile phase A: water (0.1% formic acid), mobile phase B: acetonitrile; flow rate: 60 mL/min; gradient (B %): 30% to 50% over 15 min; detector: 254 nm/220 nm; RT (min): 10.0) to afford (Compound 211) (R)-2-(5-(3H-[1,2,3]triazolo[4,5-c]pyridin-6-yl)-3-fluoropyridin-2-yl)-N-(4-isopropylphenyl)pyrrolidine-1-carboxamide (12.5 mg, 26%) as a white solid.

| Cmpd # | Characterization Data |
|---|---|
| 211 | LCMS (ESI) [M + H]⁺: 446.2 |
| | ¹H NMR (400 MHz, DMSO-d₆) δ 9.54 (s, 1H), 9.14 (s, 1H), 8.51 (s, 1H), 8.36 (dd, J = 11.6, 1.8 |
| | Hz, 1H), 8.16 (s, 1H), 7.33 (d, J = 8.4 Hz, 2H), 7.04 (d, J = 8.5 Hz, 2H), 5.39-5.33 (m, 1H), |
| | 3.83-3.74 (m, 1H), 3.67-3.59 (m, 1H), 2.77 (hept, J = 7.0 Hz, 1H), 2.39-2.32 (m, 1H), 2.17- |
| | 2.07 (m, 1H), 2.06-1.97 (m, 1H), 1.97-1.88 (m, 1H), 1.13 (d, J = 6.9 Hz, 6H). |

### Example 212: (R)-2-(5-(1H-benzo[d][1,2,3]triazol-5-yl)-3-fluoropyridin-2-yl)-N-(4-methyl-5-(1-(trifluoromethyl)cyclopropyl)pyridin-2-yl)pyrrolidine-1-carboxamide

### Step 1: 5-(4,4,5,5-tetramethyl-1,3,2-dioxaborolan-2-yl)-1-((2-(trimethylsilyl)ethoxy)methyl)-1H-benzo[d][1,2,3]triazole

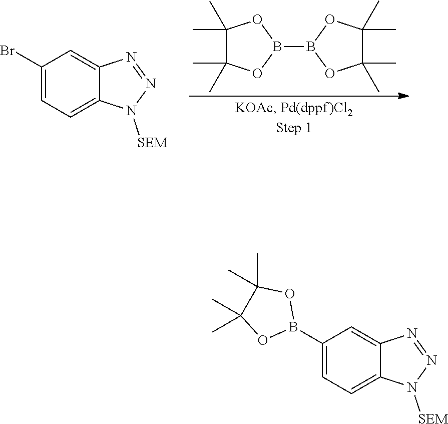

To a solution of 5-bromo-1-((2-(trimethylsilyl)ethoxy)methyl)-1H-benzo[d][1,2,3]triazole (200 mg, 0.609 mmol), bis(pinacolato)diboron (154 mg, 0.609 mmol), and potassium acetate (179 mg, 1.83 mmol) in 1,4-dioxane (3.0 mL) was added Pd(dppf)Cl₂ (44.0 mg, 0.061 mmol). The resultant mixture was heated to 90° C. for 3 hours. Upon completion, the mixture was cooled to room temperature and concentrated under reduced pressure. The crude residue was directly purified by silica gel column chromatography, eluted with petroleum ether/ethyl acetate (5:1), to afford 5-(4,4,5,5-tetramethyl-1,3,2-dioxaborolan-2-yl)-1-((2-(trimethylsilyl)ethoxy)methyl)-1H-benzo[d][1,2,3]triazole (160 mg, 69%) as a white solid. LCMS (ESI) [M+H]⁺: 376.2.

### Step 2: tert-butyl (R)-2-(3-fluoro-5-(1-((2-(trimethylsilyl)ethoxy)methyl)-1H-benzo[d][1,2,3]triazol-5-yl)pyridin-2-yl)pyrrolidine-1-carboxylate

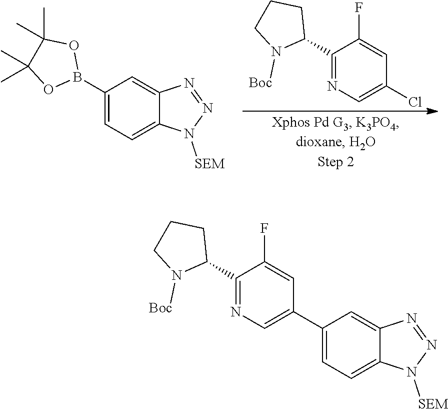 [SMILES: CC1(C)OB(c2ccc3c(c2)nnn3COCC[Si](C)(C)C)OC1(C)C ; CC(C)(C)OC(=O)N1CCC[C@@H]1c1ncc(Cl)cc1F ; CC(C)(C)OC(=O)N1CCC[C@@H]1c1ncc(-c2ccc3c(c2)nnn3COCC[Si](C)(C)C)cc1F]

To a solution of 5-(4,4,5,5-tetramethyl-1,3,2-dioxaborolan-2-yl)-1-((2-(trimethylsilyl)ethoxy)methyl)-1H-benzo[d][1,2,3]triazole (160 mg, 0.426 mmol), tert-butyl (2R)-2-(5-chloro-3-fluoropyridin-2-yl)pyrrolidine-1-carboxylate (128 mg, 0.426 mmol), and tribasic potassium phosphate (271 mg, 1.28 mmol) in 1,4-dioxane (3.0 mL) and water (0.30 mL) was added XPhos Pd G3 (36.0 mg, 0.043 mmol). The resultant mixture was heated to 80° C. for 2 hours. Upon completion, the mixture was cooled to room temperature and concentrated under reduced pressure. The crude residue was directly purified by silica gel column chromatography, eluted with petroleum ether/ethyl acetate (3:1) to afford tert-butyl (R)-2-(3-fluoro-5-(1-((2-(trimethylsilyl)ethoxy)methyl)-1H-benzo[d][1,2,3]triazol-5-yl)pyridin-2-yl)pyrrolidine-1-carboxylate (150 mg, 68%) as a white solid. LCMS (ESI) [M+H]⁺: 514.2.

### Step 3: (R)-5-(5-fluoro-6-(pyrrolidin-2-yl)pyridin-3-yl)-1H-benzo[d][1,2,3]triazole

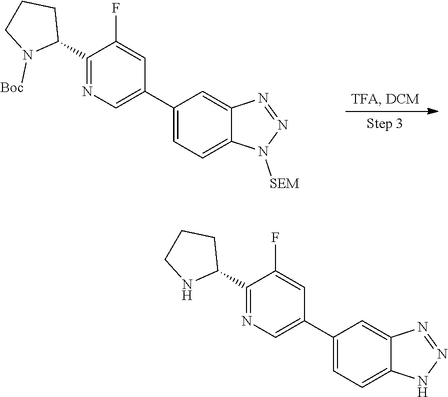 [SMILES: CC(C)(C)OC(=O)N1CCC[C@@H]1c1ncc(-c2ccc3c(c2)nnn3COCC[Si](C)(C)C)cc1F ; Fc1cc(-c2ccc3[nH]nnc3c2)cnc1[C@H]1CCCN1]

To tert-butyl (R)-2-(3-fluoro-5-(1-((2-(trimethylsilyl)ethoxy)methyl)-1H-benzo[d][1,2,3]triazol-5-yl)pyridin-2-yl)pyrrolidine-1-carboxylate (150 mg, 0.292 mmol) in dichloromethane (2.0 mL) at room temperature was added trifluoroacetic acid (1.0 mL). After 3 hours, the mixture was concentrated under reduced pressure to afford (R)-5-(5-fluoro-6-(pyrrolidin-2-yl)pyridin-3-yl)-1H-benzo[d][1,2,3]triazole (70 mg, crude) as a white solid, which was used directly in the next step without further purification. LCMS (ESI) [M+H]⁺: 284.1.

### Step 4: (R)-2-(5-(1H-benzo[d][1,2,3]triazol-5-yl)-3-fluoropyridin-2-yl)-N-(4-methyl-5-(1-(trifluoromethyl)cyclopropyl)pyridin-2-yl)pyrrolidine-1-carboxamide

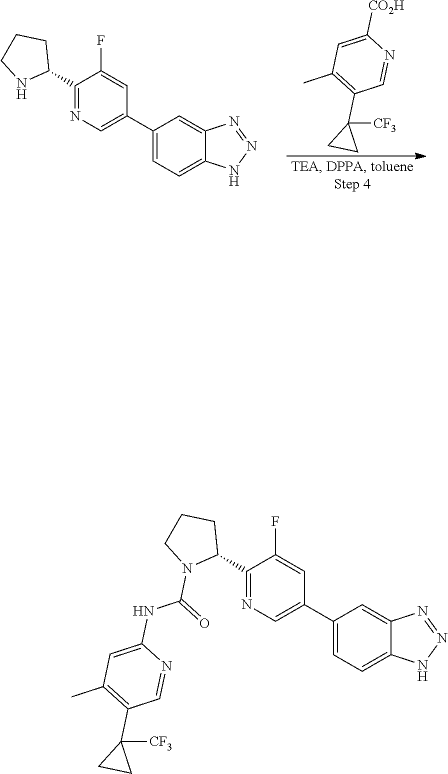 [SMILES: Fc1cc(-c2ccc3[nH]nnc3c2)cnc1[C@H]1CCCN1 ; Cc1cc(C(=O)O)ncc1C1(C(F)(F)F)CC1 ; Cc1cc(NC(=O)N2CCC[C@@H]2c2ncc(-c3ccc4[nH]nnc4c3)cc2F)ncc1C1(C(F)(F)F)CC1]

To (R)-5-(5-fluoro-6-(pyrrolidin-2-yl)pyridin-3-yl)-1H-benzo[d][1,2,3]triazole (70.0 mg, 0.176 mmol), 4-methyl-5-[1-(trifluoromethyl)cyclopropyl]pyridine-2-carboxylic acid (43 mg, 0.176 mmol), and triethylamine (53 mg, 0.528 mmol) in toluene (3.0 mL) was added diphenylphosphoryl azide (145 mg, 0.528 mmol). The resultant mixture was heated to 80° C. for 3 hours. Upon completion, the mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 60% over 20 min; detector: UV 254 nm) to afford (Compound 212) (R)-2-(5-(1H-benzo[d][1,2,3]triazol-5-yl)-3-fluoropyridin-2-yl)-N-(4-methyl-5-(1-(trifluoromethyl)cyclopropyl)pyridin-2-yl)pyrrolidine-1-carboxamide (10.8 mg, 12%) as a white solid.

| Cmpd # | Characterization Data |
|---|---|
| 212 | LCMS (ESI) [M + H]⁺: 526.2 |
| | ¹H NMR (400 MHz, methanol-d₄) δ 8.68 (s, 1H), 8.17 (s, 2H), 8.02-7.91 (m, 2H), 7.78 (d, J = 8.7 |
| | Hz, 1H), 7.68 (s, 1H), 5.52-5.42 (m, 1H), 3.94-3.83 (m, 1H), 3.81-3.68 (m, 1H), 2.54-2.43 |
| | (m, 1H), 2.39 (s, 3H), 2.25-2.17 (m, 1H), 2.17-2.03 (m, 2H), 1.48-1.40 (m, 2H), 1.09-1.00 |
| | (m, 2H). |

### Example 215: (S)-4-(6-(1-((2,2-difluorobenzo[d][1,3]dioxol-5-yl)carbamoyl)piperidin-2-yl)-5-fluoropyridin-3-yl)benzoic acid and (R)-4-(6-(1-((2,2-difluorobenzo[d][1,3]dioxol-5-yl)carbamoyl)piperidin-2-yl)-5-fluoropyridin-3-yl)benzoic acid

### Step 1: tert-butyl 2-(3-fluoro-5-(4-(methoxycarbonyl)phenyl)pyridin-2-yl)piperidine-1-carboxylate

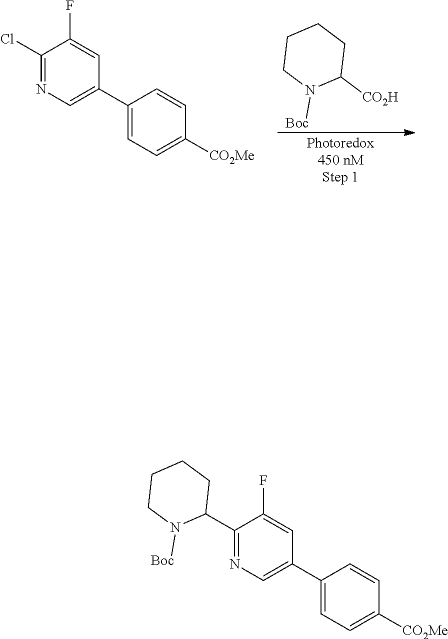 [SMILES: COC(=O)c1ccc(-c2cnc(Cl)c(F)c2)cc1 ; CC(C)(C)OC(=O)N1CCCCC1C(=O)O ; COC(=O)c1ccc(-c2cnc(C3CCCCN3C(=O)OC(C)(C)C)c(F)c2)cc1]

A solution of methyl 4-(6-chloro-5-fluoropyridin-3-yl)benzoate (5.00 g, 18.8 mmol), 1-(tert-butoxycarbonyl)piperidine-2-carboxylic acid (5.10 g, 22.6 mmol), 4,4′-dimethoxy-2,2′-bipyridine (0.500 g, 2.25 mmol), dichloronickel;1,2-dimethoxyethane (0.400 g, 1.88 mmol), phthalimide (2.80 g, 18.8 mmol), Ir[dF(CF)₃ppy]₂(dtbbpy))PF₆ (0.400 g, 0.370 mmol) and N″-tert-butyl-N,N,N′,N′-tetramethylguanidine (4.80 g, 28.2 mmol) in DMSO (100 mL) at room temperature was stirred overnight under 450 nm blue LED irradiation. Upon completion, the mixture was diluted with water (200 mL) and extracted with ethyl acetate (3×200 mL). The combined organic layers were washed with water (2×300 mL), dried over anhydrous Na₂SO₄, filtered, and concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford tert-butyl 2-(3-fluoro-5-(4-(methoxycarbonyl)phenyl)pyridin-2-yl)piperidine-1-carboxylate (1.00 g, 13%) as a yellow oil. LCMS (ESI) [M+H]⁺: 415.

### Step 2: methyl 4-(5-fluoro-6-(piperidin-2-yl)pyridin-3-yl)benzoate

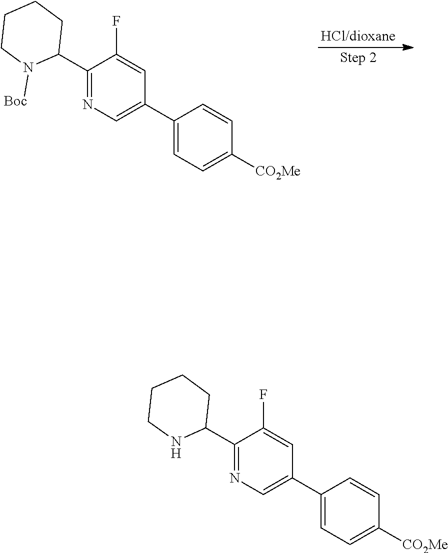 [SMILES: COC(=O)c1ccc(-c2cnc(C3CCCCN3C(=O)OC(C)(C)C)c(F)c2)cc1 ; COC(=O)c1ccc(-c2cnc(C3CCCCN3)c(F)c2)cc1]

To a solution of tert-butyl 2-(3-fluoro-5-(4-(methoxycarbonyl)phenyl)pyridin-2-yl)piperidine-1-carboxylate (1.00 g, 2.41 mmol) in 1,4-dioxane (10.0 mL) at room temperature was added a solution of hydrochloric acid (10 mL, 40 mmol, 4.0 M in 1,4-dioxane). After 2 hours, the mixture was concentrated under reduced pressure. The crude residue was directly purified by reversed-phase flash chromatography (conditions: column: C18 silica gel; mobile phase A: water (0.1% NH₄HCO₃); mobile phase B: acetonitrile; gradient (B %): 0% to 100% over 30 min; detector: UV 254 nm) to afford methyl 4-(5-fluoro-6-(piperidin-2-yl)pyridin-3-yl)benzoate (488 mg, 64%) as an orange solid. LCMS (ESI) [M+H]⁺: 315.

### Step 3: 4-(5-fluoro-6-(piperidin-2-yl)pyridin-3-yl)benzoic acid

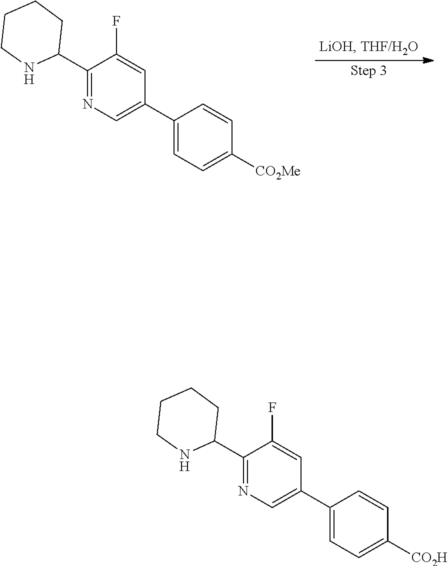 [SMILES: COC(=O)c1ccc(-c2cnc(C3CCCCN3)c(F)c2)cc1 ; O=C(O)c1ccc(-c2cnc(C3CCCCN3)c(F)c2)cc1]

To a solution of methyl 4-(5-fluoro-6-(piperidin-2-yl)pyridin-3-yl)benzoate (400 mg, 1.27 mmol) in tetrahydrofuran (5.0 mL) and water (1.0 mL) was added lithium hydroxide monohydrate (213 mg, 5.08 mmol). The mixture was heated to 60° C. for 2 hours. Upon completion, the resultant mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford 4-(5-fluoro-6-(piperidin-2-yl)pyridin-3-yl)benzoic acid (210 mg, 55%) as a white solid. LCMS (ESI) [M+H]⁺: 301.

### Step 4: 4-(6-(1-((2,2-difluorobenzo[d][1,3]dioxol-5-yl)carbamoyl)piperidin-2-yl)-5-fluoropyridin-3-yl)benzoic acid

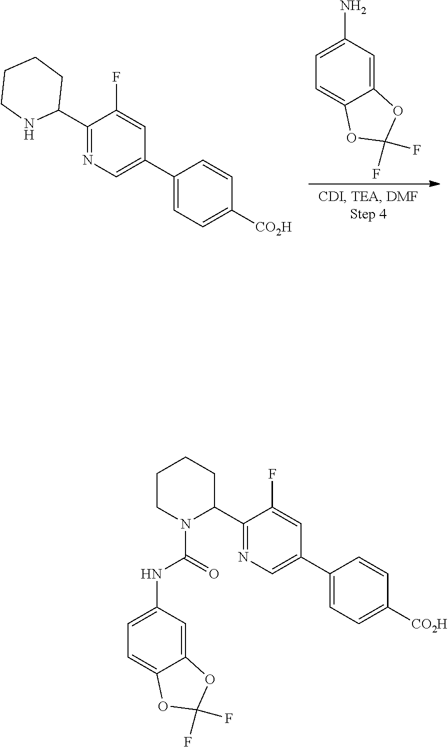 [SMILES: O=C(O)c1ccc(-c2cnc(C3CCCCN3)c(F)c2)cc1 ; Nc1ccc2c(c1)OC(F)(F)O2 ; O=C(O)c1ccc(-c2cnc(C3CCCCN3C(=O)Nc3ccc4c(c3)OC(F)(F)O4)c(F)c2)cc1]

To 4-(5-fluoro-6-(piperidin-2-yl)pyridin-3-yl)benzoic acid (60.0 mg, 0.200 mmol) in DMF (5.0 mL) was added carbonyldiimidazole (72.0 mg, 0.444 mmol) and triethylamine (70.0 mg, 0.693 mmol), and the resultant mixture was heated to 50° C. After 30 minutes, 2,2-difluorobenzo[d][1,3]dioxol-5-amine (34.0 mg, 0.200 mmol) was added, and heating was continued for an additional 2 hours. Upon completion, the mixture was concentrated under pressure. The crude residue was purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford 4-(6-(1-((2,2-difluorobenzo[d][1,3]dioxol-5-yl)carbamoyl)piperidin-2-yl)-5-fluoropyridin-3-yl)benzoic acid (50.0 mg, 50%) as a white solid. LCMS (ESI) [M+H]⁺: 500.0.

### Step 5: (S)-4-(6-(1-((2,2-difluorobenzo[d][1,3]dioxol-5-yl)carbamoyl)piperidin-2-yl)-5-fluoropyridin-3-yl)benzoic acid and (R)-4-(6-(1-((2,2-difluorobenzo[d][1,3]dioxol-5-yl)carbamoyl)piperidin-2-yl)-5-fluoropyridin-3-yl)benzoic acid

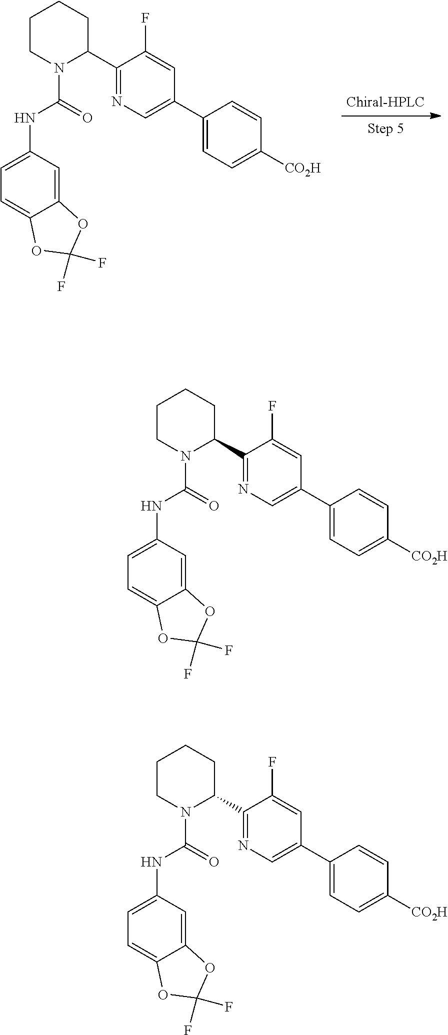 [SMILES: O=C(O)c1ccc(-c2cnc(C3CCCCN3C(=O)Nc3ccc4c(c3)OC(F)(F)O4)c(F)c2)cc1 ; O=C(O)c1ccc(-c2cnc([C@@H]3CCCCN3C(=O)Nc3ccc4c(c3)OC(F)(F)O4)c(F)c2)cc1 ; O=C(O)c1ccc(-c2cnc([C@H]3CCCCN3C(=O)Nc3ccc4c(c3)OC(F)(F)O4)c(F)c2)cc1]

4-(6-(1-((2,2-difluorobenzo[d][1,3]dioxol-5-yl)carbamoyl)piperidin-2-yl)-5-fluoropyridin-3-yl)benzoic acid (50 mg) was resolved by preparative chiral HPLC (conditions: column: CHIRALPAK IA, 3*25 cm, 5 μm; mobile phase A: hexane/MTBE (1:1; 0.1% formic acid); mobile phase B: ethanol; flow rate: 40 mL/min; gradient (B %): isocratic, 50% B; detector: 220/254 nm; RT1 (min): 6.3; RT2 (min): 12.8) to afford (Compound 215a; Method BF, Peak 1, 0.54 min) (S)-4-(6-(1-((2,2-difluorobenzo[d][1,3]dioxol-5-yl)carbamoyl)piperidin-2-yl)-5-fluoropyridin-3-yl)benzoic acid (1^{st} peak, 14.0 mg, 29%) as a white solid and (Compound 215b; Method BF, Peak 2, 1.69 min) (R)-4-(6-(1-((2,2-difluorobenzo[d][1,3]dioxol-5-yl)carbamoyl)piperidin-2-yl)-5-fluoropyridin-3-yl)benzoic acid (2^{nd} peak, 15.0 mg, 31%) as a white solid.

| Cmpd # | Characterization Data |
|---|---|
| 215a | LCMS (ESI) [M + H]⁺: 501.2 |
| | ¹H NMR (400 MHz, DMSO-d₆) δ 8.83-8.70 (m, 2H), 8.11 (d, J = 12.0 Hz, 1H), 8.03 (d, J = 8.4 |
| | Hz, 2H), 7.91 (d, J = 8.4 Hz, 2H), 7.57 (d, J = 2.2 Hz, 1H), 7.24 (d, J = 8.8 Hz, 1H), 7.17 (dd, J = |
| | 8.8, 2.1 Hz, 1H), 5.74 (d, J = 5.8 Hz, 1H), 4.12-4.00 (m, 1H), 3.69-3.63 (m, 1H), 2.21-2.10 |
| | (m, 1H), 1.98-1.88 (m, 1H), 1.81-1.72 (m, 1H), 1.67-1.51 (m, 2H), 1.48-1.35 (m, 1H). |
| 215b | LCMS (ESI) [M + H]⁺: 501.2 |
| | ¹H NMR (400 MHz, DMSO-d₆) δ 8.82-8.72 (m, 2H), 8.11 (dd, J = 11.9, 1.9 Hz, 1H), 8.03 (d, J = |
| | 8.0 Hz, 2H), 7.91 (d, J = 8.1 Hz, 2H), 7.57 (s, 1H), 7.24 (d, J = 8.7 Hz, 1H), 7.17 (d, J = 8.8 Hz, |
| | 1H), 5.78-5.69 (m, 1H), 4.13-4.01 (m, 1H), 3.72-3.60 (m, 1H), 2.21-2.11 (m, 1H), 2.01- |
| | 1.88 (m, 1H), 1.82-1.72 (m, 1H), 1.67-1.52 (m, 2H), 1.48-1.39 (m, 1H). |

### Example 216: (R)-4-(6-(4-((2,2-difluorobenzo[d][1,3]dioxol-5-yl)carbamoyl)morpholin-3-yl)-5-fluoropyridin-3-yl)benzoic acid and (S)-4-(6-(4-((2,2-difluorobenzo[d][1,3]dioxol-5-yl)carbamoyl)morpholin-3-yl)-5-fluoropyridin-3-yl)benzoic acid

### Step 1: tert-butyl 3-(3-fluoro-5-(4-(methoxycarbonyl)phenyl)pyridin-2-yl)morpholine-4-carboxylate

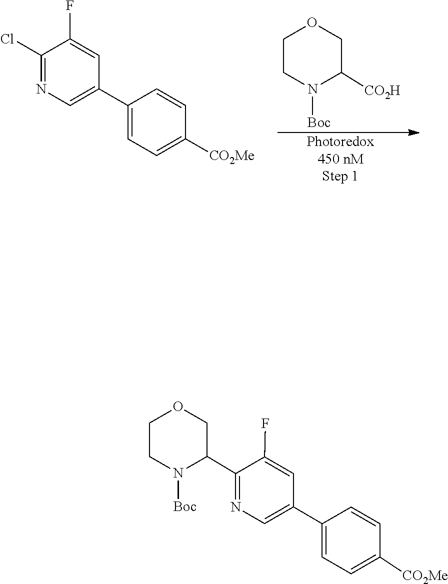 [SMILES: COC(=O)c1ccc(-c2cnc(Cl)c(F)c2)cc1 ; CC(C)(C)OC(=O)N1CCOCC1C(=O)O ; COC(=O)c1ccc(-c2cnc(C3COCCN3C(=O)OC(C)(C)C)c(F)c2)cc1]

To a solution of methyl 4-(6-chloro-5-fluoropyridin-3-yl)benzoate (4.00 g, 15.0 mmol) in DMSO (100 mL) was added sequentially 1-4-(tert-butoxycarbonyl)morpholine-3-carboxylic acid (4.10 g, 18.1 mmol), 4,4′-dimethoxy-2,2′-bipyridine (0.300 g, 1.80 mmol), dichloronickel;1,2-dimethoxyethane (0.300 g, 1.50 mmol), 2,3-dihydro-1H-isoindole-1,3-dione (2.20 g, 15.0 mmol), Ir[dF(CF)₃ppy]₂(dtbbpy))PF₆ (0.300 g, 0.300 mmol) and N″-tert-butyl-N,N,N′,N′-tetramethylguanidine (3.80 g, 22.6 mmol), and the resultant mixture was stirred at room temperature overnight under 450 nm blue LED irradiation. Upon completion, the mixture was diluted with water (200 mL) and extracted with ethyl acetate (3×200 mL). The combined organic layers were washed with water (2×300 mL), dried over anhydrous Na₂SO₄, filtered, and concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford tert-butyl 3-(3-fluoro-5-(4-(methoxycarbonyl)phenyl)pyridin-2-yl)morpholine-4-carboxylate (1.20 g, 19%) as a white solid. LCMS (ESI) [M+H]⁺: 417.

### Step 2: 4-(6-(4-(tert-butoxycarbonyl)morpholin-3-yl)-5-fluoropyridin-3-yl)benzoic

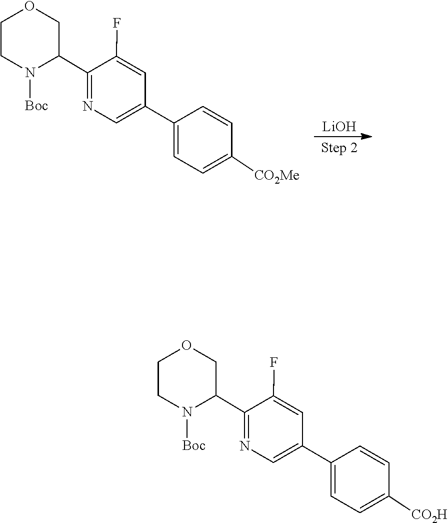 [SMILES: COC(=O)c1ccc(-c2cnc(C3COCCN3C(=O)OC(C)(C)C)c(F)c2)cc1 ; "LiOH" ; CC(C)(C)OC(=O)N1CCOCC1c1ncc(-c2ccc(C(=O)O)cc2)cc1F]

To a solution of tert-butyl 3-(3-fluoro-5-(4-(methoxycarbonyl)phenyl)pyridin-2-yl)morpholine-4-carboxylate (700 mg, 2.21 mmol) in tetrahydrofuran (7.0 mL) and water (4.0 mL) was added lithium hydroxide monohydrate (372 mg, 8.85 mmol) at room temperature. After 2 hours, the mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford 4-(6-(4-(tert-butoxycarbonyl)morpholin-3-yl)-5-fluoropyridin-3-yl)benzoic acid (300 mg, 45%) as a white solid. LCMS (ESI) [M+H]⁺: 403.0.

### Step 3: 4-(5-fluoro-6-(morpholin-3-yl)pyridin-3-yl)benzoic acid

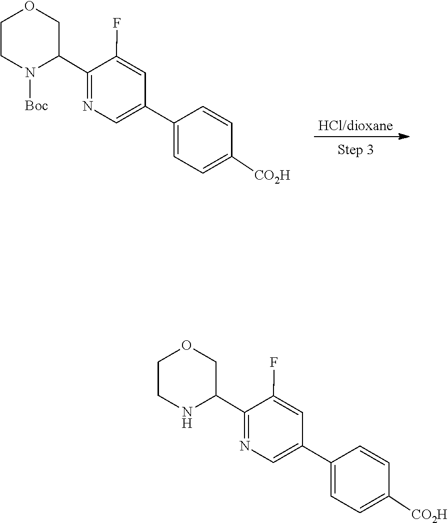 [SMILES: CC(C)(C)OC(=O)N1CCOCC1c1ncc(-c2ccc(C(=O)O)cc2)cc1F ; "HCl/dioxane" ; O=C(O)c1ccc(-c2cnc(C3COCCN3)c(F)c2)cc1]

To a solution of 4-(6-(4-(tert-butoxycarbonyl)morpholin-3-yl)-5-fluoropyridin-3-yl)benzoic acid (300 mg, 0.740 mmol) in 1,4-dioxane (10.0 mL) at room temperature was added a solution of hydrochloric acid (10.0 mL, 40.0 mmol, 4.0 M in 1,4-dioxane). After 1 hour, the mixture was concentrated under reduced pressure to afford 4-(5-fluoro-6-(morpholin-3-yl)pyridin-3-yl)benzoic acid (200 mg, crude) as a white solid, which was used directly in the next step without further purification. LCMS (ESI) [M+H]⁺: 303.0.

### Step 4: 4-(6-(4-((2,2-difluorobenzo[d][1,3]dioxol-5-yl)carbamoyl)morpholin-3-yl)-5-fluoropyridin-3-yl)benzoic acid

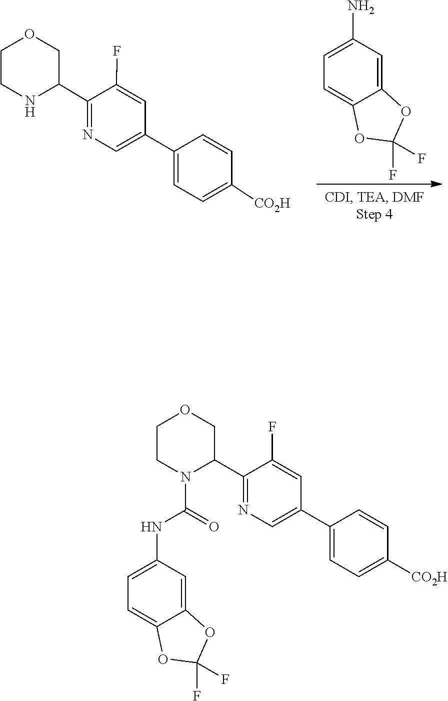 [SMILES: O=C(O)c1ccc(-c2cnc(C3COCCN3)c(F)c2)cc1 ; Nc1ccc2c(c1)OC(F)(F)O2 ; O=C(O)c1ccc(-c2cnc(C3COCCN3C(=O)Nc3ccc4c(c3)OC(F)(F)O4)c(F)c2)cc1]

To a solution of 4-(5-fluoro-6-(morpholin-3-yl)pyridin-3-yl)benzoic acid (40.0 mg, 0.130 mmol) in DMF (0.300 mL) was added triethylamine (40.0 mg, 0.309 mmol) and CDI (42.0 mg, 0.260 mmol) and the resultant mixture was heated to 50° C. After 30 minutes, the reaction was cooled to room temperature and 2,2-difluoro-1,3-benzodioxol-5-amine (22.0 mg, 0.130 mmol) was added. The resultant mixture was stirred at room temperature for an additional 30 minutes. Upon completion, the reaction mixture was directly purified by reversed-phase flash chromatography (conditions: column: C18 silica gel; mobile phase A: water (0.1% NH₄HCO₃); mobile phase B: acetonitrile; gradient (B %): 0% to 100% over 30 min; detector: UV 254 nm) to afford 4-(6-(4-((2,2-difluorobenzo[d][1,3]dioxol-5-yl)carbamoyl)morpholin-3-yl)-5-fluoropyridin-3-yl)benzoic acid (49 mg, 74%) as a white solid. LCMS (ESI) [M+H]⁺: 502.2.

### Step 5: (R)-4-(6-(4-((2,2-difluorobenzo[d][1,3]dioxol-5-yl)carbamoyl)morpholin-3-yl)-5-fluoropyridin-3-yl)benzoic acid and (S)-4-(6-(4-((2,2-difluorobenzo[d][1,3]dioxol-5-yl)carbamoyl)morpholin-3-yl)-5-fluoropyridin-3-yl)benzoic acid

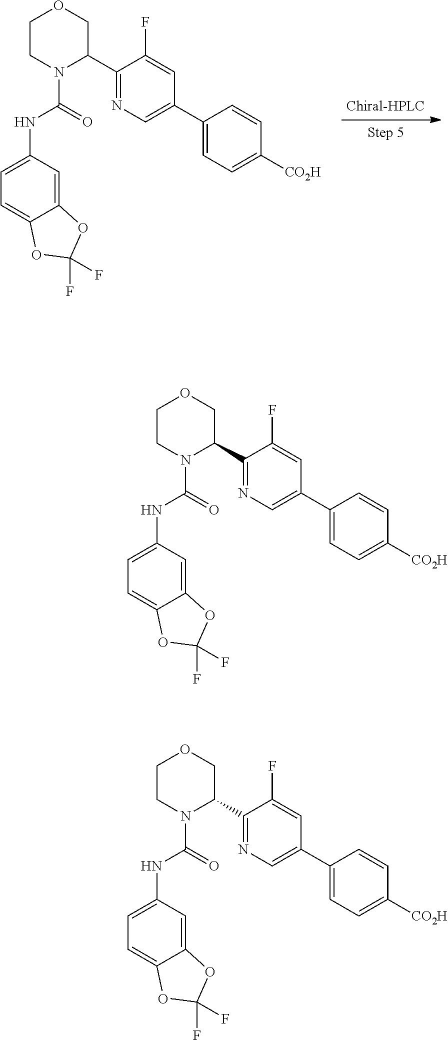 [SMILES: O=C(O)c1ccc(-c2cnc(C3COCCN3C(=O)Nc3ccc4c(c3)OC(F)(F)O4)c(F)c2)cc1 ; O=C(O)c1ccc(-c2cnc([C@@H]3COCCN3C(=O)Nc3ccc4c(c3)OC(F)(F)O4)c(F)c2)cc1 ; O=C(O)c1ccc(-c2cnc([C@H]3COCCN3C(=O)Nc3ccc4c(c3)OC(F)(F)O4)c(F)c2)cc1]

4-(6-(4-((2,2-difluorobenzo[d][1,3]dioxol-5-yl)carbamoyl)morpholin-3-yl)-5-fluoropyridin-3-yl)benzoic acid (49.0 mg) was resolved by preparative chiral HPLC (conditions: column: CHIRALPAK IA, 3*25 cm, 5 μm; mobile phase A: hexane/MTBE (1:1; 0.1% formic acid), mobile phase B: methanol; flow rate: 40 mL/min; gradient (B %): isocratic, 40% B; detector: 220/254 nm; RT1 (min): 6.0; RT2 (min): 12.5) to afford (Compound 216a; Method AV, Peak 1, 0.64 min) (R)-4-(6-(4-((2,2-difluorobenzo[d][1,3]dioxol-5-yl)carbamoyl)morpholin-3-yl)-5-fluoropyridin-3-yl)benzoic acid (1^{st} peak, 16.0 mg, 33%) as a white solid and (Compound 216b; Method AV, Peak 2, 1.83 min) (R)-4-(6-(1-((2,2-difluorobenzo[d][1,3]dioxol-5-yl)carbamoyl)piperidin-2-yl)-5-fluoropyridin-3-yl)benzoic acid (2^{nd} peak, 8.5 mg, 17%) as a white solid.

| Cmpd # | Characterization Data |
|---|---|
| 216a | LCMS (ESI) [M + H]⁺: 502.2 |
| | ¹H NMR (400 MHz, methanol-d₄) δ 8.73-8.66 (m, 1H), 8.13 (dd, J = 8.4, 1.8 Hz, 2H), 7.97- |
| | 7.86 (m, 1H), 7.83-7.75 (m, 2H), 7.37-7.30 (m, 1H), 7.10-7.00 (m, 2H), 5.51 (s, 1H), 4.42 |
| | (d, J = 12.0 Hz, 1H), 4.13-3.94 (m, 3H), 3.89-3.79 (m, 1H), 3.75-3.67 (m, 1H). |
| 216b | LCMS (ESI) [M + H]⁺: 502.2 |
| | ¹H NMR (400 MHz, methanol-d₄) δ 8.73-8.66 (m, 1H), 8.17-8.07 (m, 2H), 7.94-7.84 (m, |
| | 1H), 7.84-7.73 (m, 2H), 7.34 (s, 1H), 7.05 (s, 2H), 5.51 (d, J = 3.6 Hz, 1H), 4.42 (d, J = 12.0 |
| | Hz, 1H), 4.13-3.96 (m, 3H), 3.88-3.80 (m, 1H), 3.76-3.64 (m, 1H). |

### Example 217: (R)-3-(5-(1H-benzo[d][1,2,3]triazol-5-yl)-3-fluoropyridin-2-yl)-N-(2,2-difluorobenzo[d][1,3]dioxol-5-yl)morpholine-4-carboxamide and (S)-3-(5-(1H-benzo[d][1,2,3]triazol-5-yl)-3-fluoropyridin-2-yl)-N-(2,2-difluorobenzo[d][1,3]dioxol-5-yl)morpholine-4-carboxamide

### Step 1: tert-butyl 3-(5-chloro-3-fluoropyridin-2-yl)morpholine-4-carboxylate

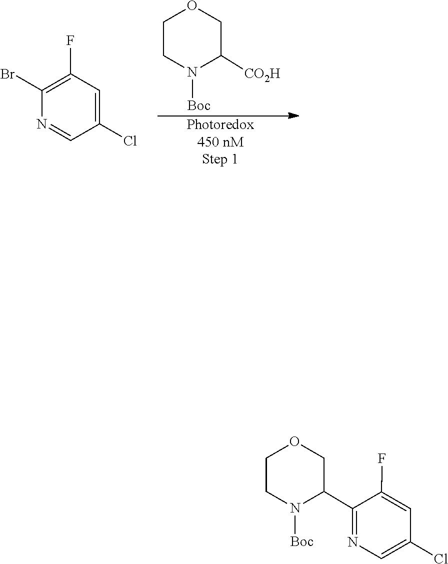 [SMILES: Fc1cc(Cl)cnc1Br ; CC(C)(C)OC(=O)N1CCOCC1C(=O)O ; CC(C)(C)OC(=O)N1CCOCC1c1ncc(Cl)cc1F]

To a solution of 2-bromo-5-chloro-3-fluoropyridine (1.00 g, 4.75 mmol) in DMF (10.0 mL) at room temperature was added sequentially 4-(tert-butoxycarbonyl)morpholine-3-carboxylic acid (1.10 g, 4.75 mmol), cesium carbonate (4.60 g, 14.3 mmol), NiBr₂·Bipy (178 mg, 0.470 mmol), and 4CzIPN (93.7 mg, 0.110 mmol), and the resultant mixture was stirred for 12 hours under 450 nm blue LED irradiation. Upon completion, the mixture was poured into water (100 mL) and extracted with ethyl acetate (3×30 mL). The combined organic layers were dried over anhydrous Na₂SO₄, filtered, and concentrated under reduced pressure. The crude residue was purified by silica gel column chromatography, eluted with petroleum ether/ethyl acetate (5:1), to afford tert-butyl 3-(5-chloro-3-fluoropyridin-2-yl)morpholine-4-carboxylate (480 mg, 31%) as a yellow oil. LCMS (ESI) [M+H]⁺: 317.0.

### Step 2: 3-(5-chloro-3-fluoropyridin-2-yl)morpholine

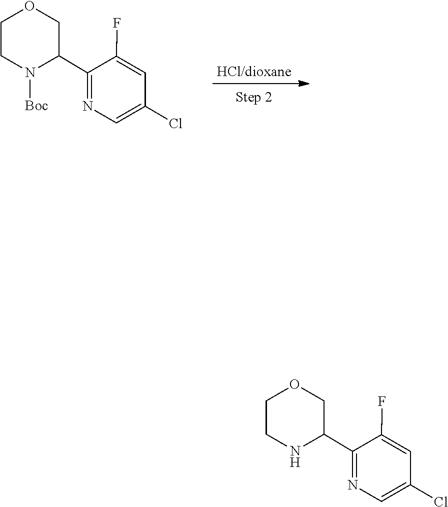 [SMILES: CC(C)(C)OC(=O)N1CCOCC1c1ncc(Cl)cc1F ; "HCl/dioxane" ; Fc1cc(Cl)cnc1C1COCCN1]

To tert-butyl 3-(5-chloro-3-fluoropyridin-2-yl)morpholine-4-carboxylate (470 mg, 1.48 mmol) in 1,4-dioxane (5 mL) was added a solution of hydrochloric acid (0.700 mL, 3.20 mmol, 4.0 M in 1,4-dioxane) at room temperature. After 2 hours, the mixture was concentrated under reduced pressure to afford 3-(5-chloro-3-fluoropyridin-2-yl)morpholine (300 mg, crude) as a pale-yellow solid, which was used directly in the next step without further purification. LCMS (ESI) [M+H]⁺: 217.1.

### Step 3: 3-(5-chloro-3-fluoropyridin-2-yl)-N-(2,2-difluorobenzo[d][1,3]dioxol-5-yl)morpholine-4-carboxamide

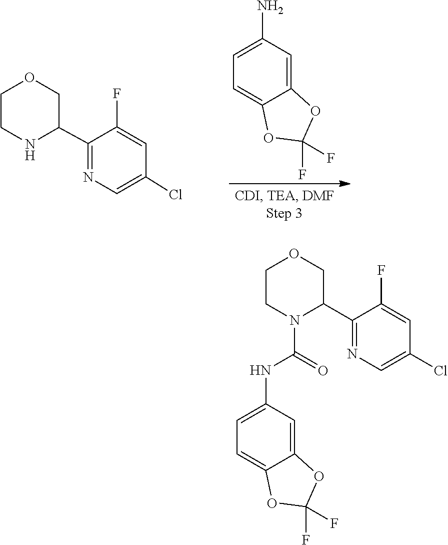 [SMILES: Fc1cc(Cl)cnc1C1COCCN1 ; Nc1ccc2c(c1)OC(F)(F)O2 ; O=C(Nc1ccc2c(c1)OC(F)(F)O2)N1CCOCC1c1ncc(Cl)cc1F]

To a solution of 2,2-difluoro-1,3-benzodioxol-5-amine (240 mg, 1.38 mmol) in DMF (3.0 mL) was added CDI (150 mg, 0.920 mmol), triethylamine (187 mg, 1.84 mmol) and 3-(5-chloro-3-fluoropyridin-2-yl)morpholine (200 mg, 0.920 mmol). The resultant mixture was heated to 40° C. for 2 hours. Upon completion, the mixture was concentrated under reduced pressure. The crude residue was purified by reversed-phase flash chromatography (conditions: column: C18 silica gel; mobile phase A: water (0.1% NH₄HCO₃); mobile phase B: acetonitrile; gradient (B %): 0% to 100% over 30 min; detector: UV 254 nm) to afford 3-(5-chloro-3-fluoropyridin-2-yl)-N-(2,2-difluorobenzo[d][1,3]dioxol-5-yl)morpholine-4-carboxamide (300 mg, 78%) as a pale yellow semi-solid. LCMS (ESI) [M+H]⁺: 416.0.

### Step 4: N-(2,2-difluorobenzo[d][1,3]dioxol-5-yl)-3-(3-fluoro-5-(1-((2-(trimethylsilyl)ethoxy)methyl)-1H-benzo[d][1,2,3]triazol-5-yl)pyridin-2-yl)morpholine-4-carboxamide

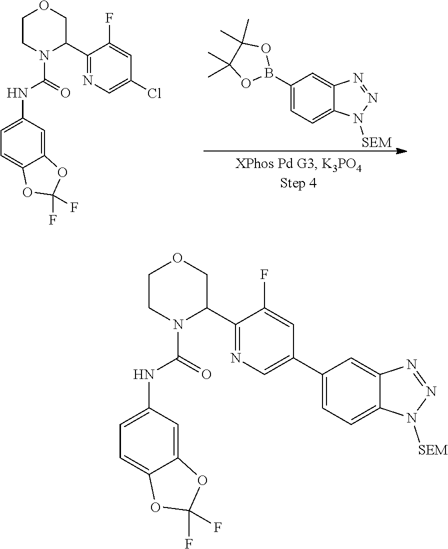 [SMILES: O=C(Nc1ccc2c(c1)OC(F)(F)O2)N1CCOCC1c1ncc(Cl)cc1F ; CC1(C)OB(c2ccc3c(c2)nnn3COCC[Si](C)(C)C)OC1(C)C ; C[Si](C)(C)CCOCn1nnc2cc(-c3cnc(C4COCCN4C(=O)Nc4ccc5c(c4)OC(F)(F)O5)c(F)c3)ccc21]

To a solution of 3-(5-chloro-3-fluoropyridin-2-yl)-N-(2,2-difluorobenzo[d][1,3]dioxol-5-yl)morpholine-4-carboxamide (120 mg, 0.280 mmol) in tetrahydrofuran (3.0 mL) and water (0.30 mL) was added 5-(4,4,5,5-tetramethyl-1,3,2-dioxaborolan-2-yl)-1-((2-(trimethylsilyl)ethoxy)methyl)-1H-benzo[d][1,2,3]triazole (108 mg, 0.28 mmol), tribasic potassium phosphate (184 mg, 0.860 mmol), and XPhos Pd G3 (24.4 mg, 0.020 mmol). The resultant mixture was heated to 85° C. for 2 hours. Upon completion, the mixture was concentrated under reduced pressure. The crude residue was purified by reversed-phase flash chromatography (conditions: column: C18 silica gel; mobile phase A: water (0.1% formic acid); mobile phase B: acetonitrile; gradient (B %): 0% to 100% over 30 min; detector: UV 254 nm) to afford N-(2,2-difluorobenzo[d][1,3]dioxol-5-yl)-3-(3-fluoro-5-(1-((2-(trimethylsilyl)ethoxy)methyl)-1H-benzo[d][1,2,3]triazol-5-yl)pyridin-2-yl)morpholine-4-carboxamide (136 mg, 74%) as a pale yellow semi-solid. LCMS (ESI) [M+H]⁺: 629.2.

### Step 5: 3-(5-(1H-benzo[d][1,2,3]triazol-5-yl)-3-fluoropyridin-2-yl)-N-(2,2-difluorobenzo[d][1,3]dioxol-5-yl)morpholine-4-carboxamide

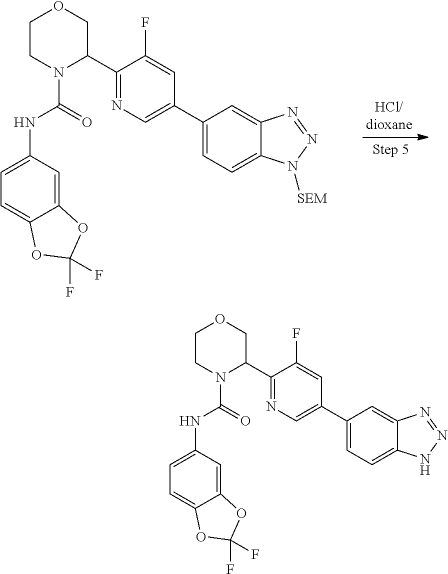 [SMILES: C[Si](C)(C)CCOCn1nnc2cc(-c3cnc(C4COCCN4C(=O)Nc4ccc5c(c4)OC(F)(F)O5)c(F)c3)ccc21 ; O=C(Nc1ccc2c(c1)OC(F)(F)O2)N1CCOCC1c1ncc(-c2ccc3[nH]nnc3c2)cc1F]

To a solution of N-(2,2-difluorobenzo[d][1,3]dioxol-5-yl)-3-(3-fluoro-5-(1-((2-(trimethylsilyl)ethoxy)methyl)-1H-benzo[d][1,2,3]triazol-5-yl)pyridin-2-yl)morpholine-4-carboxamide (130 mg, 0.200 mmol) in 1,4-dioxane (3.0 mL) was added a solution of hydrochloric acid (2.5 mL, 10.0 mmol, 4.0 M in 1,4-dioxane) at room temperature. After 3 hours, the mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0%-100% over 30 min; detector: UV 254 nm) to afford 3-(5-(1H-benzo[d][1,2,3]triazol-5-yl)-3-fluoropyridin-2-yl)-N-(2,2-difluorobenzo[d][1,3]dioxol-5-yl)morpholine-4-carboxamide (100 mg, 97%) as a white solid. LCMS (ESI) [M+H]⁺: 499.1.

### Step 6: (R)-3-(5-(1H-benzo[d][1,2,3]triazol-5-yl)-3-fluoropyridin-2-yl)-N-(2,2-difluorobenzo[d][1,3]dioxol-5-yl)morpholine-4-carboxamide and (S)-3-(5-(1H-benzo[d][1,2,3]triazol-5-yl)-3-fluoropyridin-2-yl)-N-(2,2-difluorobenzo[d][1,3]dioxol-5-yl)morpholine-4-carboxamide

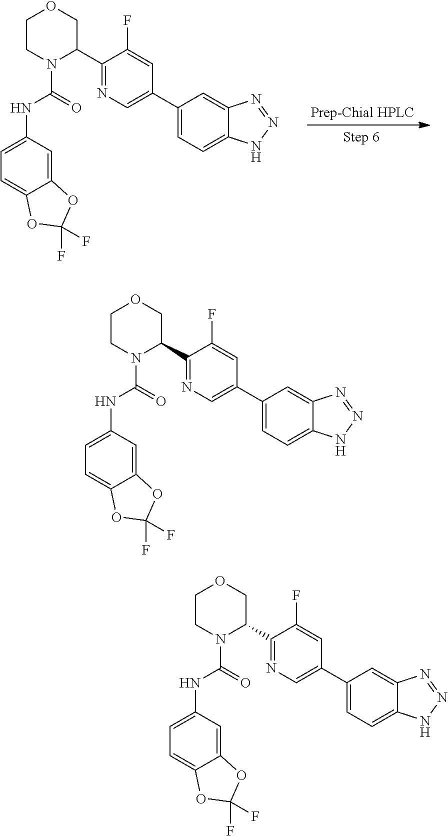 [SMILES: O=C(Nc1ccc2c(c1)OC(F)(F)O2)N1CCOCC1c1ncc(-c2ccc3[nH]nnc3c2)cc1F ; O=C(Nc1ccc2c(c1)OC(F)(F)O2)N1CCOC[C@H]1c1ncc(-c2ccc3[nH]nnc3c2)cc1F ; O=C(Nc1ccc2c(c1)OC(F)(F)O2)N1CCOC[C@@H]1c1ncc(-c2ccc3[nH]nnc3c2)cc1F]

3-(5-(1H-benzo[d][1,2,3]triazol-5-yl)-3-fluoropyridin-2-yl)-N-(2,2-difluorobenzo[d][1,3]dioxol-5-yl)morpholine-4-carboxamide (100 mg) was resolved by preparative chiral HPLC (conditions: column: CHIRALPAK IC, 2*25 cm, 5 μm; mobile phase A: hexane (0.1% formic acid), mobile phase B: ethanol; flow rate: 20 mL/min; gradient (B %): isocratic, 50%; detector: 220/254 nm; RT1 (min): 3.7; RT2 (min): 5.8) to afford (Compound 217a; Method BG, Peak 1, 0.55 min) (R)-3-(5-(1H-benzo[d][1,2,3]triazol-5-yl)-3-fluoropyridin-2-yl)-N-(2,2-difluorobenzo[d][1,3]dioxol-5-yl)morpholine-4-carboxamide (1^{st} peak, 27.2 mg, 27%) as a white solid and (Compound 217b; Method BG, Peak 2, 0.84 min) (S)-3-(5-(1H-benzo[d][1,2,3]triazol-5-yl)-3-fluoropyridin-2-yl)-N-(2,2-difluorobenzo[d][1,3]dioxol-5-yl)morpholine-4-carboxamide (2^{nd} peak, 26.5 mg, 26%) as a white solid.

| Cmpd # | Characterization Data |
|---|---|
| 217a | LCMS (ESI) [M + H]⁺: 499.2 |
| | ¹H NMR (400 MHz, DMSO-d₆) δ 8.83 (d, J = 8.5 Hz, 2H), 8.32 (s, 1H), 8.18 (d, J = 12.1 Hz, |
| | 1H), 8.01 (d, J = 8.7 Hz, 1H), 7.83 (d, J = 8.4 Hz, 1H), 7.58 (d, J = 2.0 Hz, 1H), 7.26 (d, J = 8.8 |
| | Hz, 1H), 7.17 (dd, J = 8.8, 2.1 Hz, 1H), 5.47 (d, J = 3.6 Hz, 1H), 4.28 (d, J = 11.9 Hz, 1H), 4.06- |
| | 3.88 (m, 4H), 3.64-3.55 (m, 1H). |
| 217b | LCMS (ESI) [M + H]⁺: 499.1 |
| | ¹H NMR (400 MHz, DMSO-d₆) δ 8.83 (d, J = 7.2 Hz, 2H), 8.33 (s, 1H), 8.19 (dd, J = 12.1, 1.9 |
| | Hz, 1H), 8.01 (d, J = 8.6 Hz, 1H), 7.84 (dd, J = 8.6, 1.6 Hz, 1H), 7.58 (d, J = 2.1 Hz, 1H), 7.26 |
| | (d, J = 8.8 Hz, 1H), 7.17 (dd, J = 8.8, 2.1 Hz, 1H), 5.47 (d, J = 3.4 Hz, 1H), 4.28 (d, J = 11.9 Hz, |
| | 1H), 4.05-3.81 (m, 4H), 3.68-3.52 (m, 1H). |

### Example 218: (R)—N-(2,2-difluorobenzo[d][1,3]dioxol-5-yl)-2-(3-fluoro-5-(4-((trifluoromethyl)sulfonamido)phenyl)pyridin-2-yl)pyrrolidine-1-carboxamide

### Step 1: 1,1,1-trifluoro-N-(4-(4,4,5,5-tetramethyl-1,3,2-dioxaborolan-2-yl)phenyl)methanesulfonamide

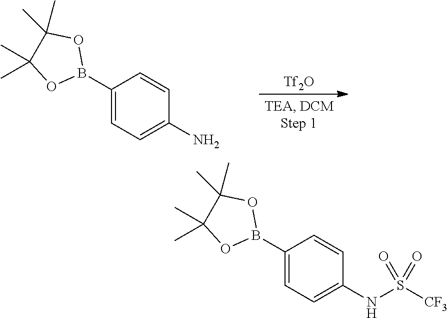

A solution of 4-(4,4,5,5-tetramethyl-1,3,2-dioxaborolan-2-yl)aniline (300 mg, 1.37 mmol), trifluoromethanesulfonic anhydride (386 mg, 1.37 mmol) and triethylamine (415 mg, 4.10 mmol) in dichloromethane (3.0 mL) was stirred at 25° C. for 3 hours. Upon completion, the mixture was concentrated under reduced pressure. The crude residue was directly purified by silica gel column chromatography, eluted with petroleum ether/ethyl acetate (3:1), to afford 1,1,1-trifluoro-N-(4-(4,4,5,5-tetramethyl-1,3,2-dioxaborolan-2-yl)phenyl)methanesulfonamide (160 mg, 33%) as a yellow solid. LCMS (ESI) [M+H]⁺: 352.0.

### Step 2: (R)-5-chloro-3-fluoro-2-(pyrrolidin-2-yl)pyridine

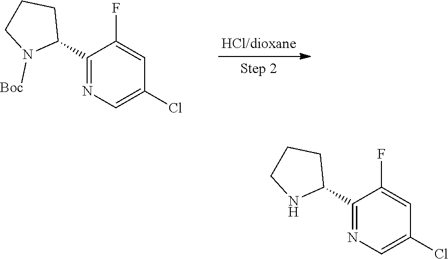 [SMILES: CC(C)(C)OC(=O)N1CCC[C@@H]1c1ncc(Cl)cc1F ; Fc1cc(Cl)cnc1[C@H]1CCCN1]

To a solution of tert-butyl (R)-2-(5-chloro-3-fluoropyridin-2-yl)pyrrolidine-1-carboxylate (500 mg, 1.66 mmol) in 1,4-dioxane (2.0 mL) was added a solution of hydrochloric acid (5.0 mL, 20 mmol, 4.0 M in 1,4-dioxane) at room temperature. After 3 hours, the reaction mixture was concentrated under reduced pressure to afford (R)-5-chloro-3-fluoro-2-(pyrrolidin-2-yl)pyridine (300 mg, 89% crude) as a white solid, which was used directly in the next step without further purification. LCMS (ESI) [M+H]⁺: 201.0.

### Step 3: (R)-2-(5-chloro-3-fluoropyridin-2-yl)-N-(2,2-difluorobenzo[d][1,3]dioxol-5-yl)pyrrolidine-1-carboxamide

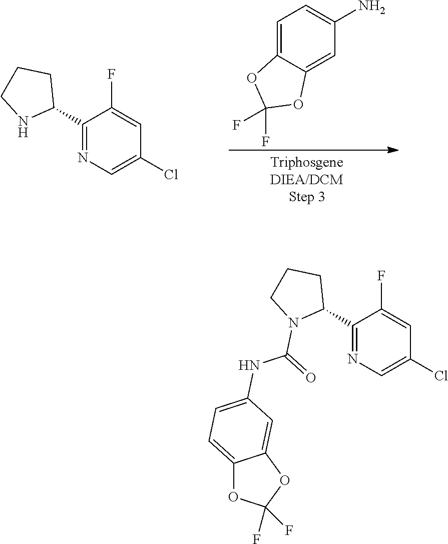 [SMILES: Fc1cc(Cl)cnc1[C@H]1CCCN1 ; Nc1ccc2c(c1)OC(F)(F)O2 ; O=C(Nc1ccc2c(c1)OC(F)(F)O2)N1CCC[C@@H]1c1ncc(Cl)cc1F]

A solution of (R)-5-chloro-3-fluoro-2-(pyrrolidin-2-yl)pyridine (200 mg, 0.996 mmol), 2,2-difluoro-1,3-benzodioxol-5-amine (164 mg, 0.996 mmol), triphosgene (120 mg, 0.400 mmol), and N, N-diisopropylethylamine (644 mg, 4.98 mmol) in dichloromethane (2.0 mL) was stirred at 25° C. for 3 h. Upon completion, the mixture was concentrated under reduced pressure, and the crude residue was purified by silica gel column chromatography, eluted with petroleum ether/ethyl acetate (1:1) to afford (R)-2-(5-chloro-3-fluoropyridin-2-yl)-N-(2,2-difluorobenzo[d][1,3]dioxol-5-yl)pyrrolidine-1-carboxamide (200 mg, 50%) as a white solid. LCMS (ESI) [M+H]⁺: 400.0.

### Step 4: (R)—N-(2,2-difluorobenzo[d][1,3]dioxol-5-yl)-2-(3-fluoro-5-(4-((trifluoromethyl)sulfonamido)phenyl)pyridin-2-yl)pyrrolidine-1-carboxamide

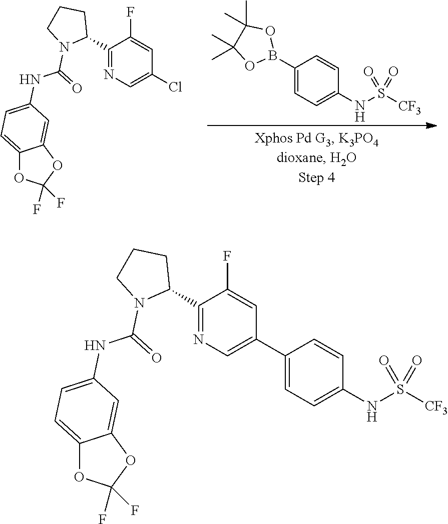 [SMILES: O=C(Nc1ccc2c(c1)OC(F)(F)O2)N1CCC[C@@H]1c1ncc(Cl)cc1F ; CC1(C)OB(c2ccc(NS(=O)(=O)C(F)(F)F)cc2)OC1(C)C ; O=C(Nc1ccc2c(c1)OC(F)(F)O2)N1CCC[C@@H]1c1ncc(-c2ccc(NS(=O)(=O)C(F)(F)F)cc2)cc1F]

A solution of (R)-2-(5-chloro-3-fluoropyridin-2-yl)-N-(2,2-difluorobenzo[d][1,3]dioxol-5-yl)pyrrolidine-1-carboxamide (100 mg, 0.250 mmol), 1,1,1-trifluoro-N-[4-(4,4,5,5-tetramethyl-1,3,2-dioxaborolan-2-yl)phenyl]methanesulfonamide (87.0 mg, 0.250 mmol), tribasic potassium phosphate (159 mg, 0.750 mmol) and XPhos Pd G3 (21.0 mg, 0.025 mmol) in 1,4-dioxane (1.0 mL) and water (0.10 mL) was stirred at 90° C. overnight. Upon completion, the reaction mixture was concentrated under reduced pressure. The crude residue was directly purified by silica gel column chromatography, eluted with dichloromethane/methanol (10:1) to afford (Compound 218) (R)—N-(2,2-difluorobenzo[d][1,3]dioxol-5-yl)-2-(3-fluoro-5-(4-((trifluoromethyl)sulfonamido)phenyl)pyridin-2-yl)pyrrolidine-1-carboxamide (34.1 mg, 23%) as a white solid.

| Cmpd # | Characterization Data |
|---|---|
| 218 | LCMS (ESI) [M + H]⁺: 589.1 |
| | ¹H NMR (400 MHz, methanol-d₄) δ 8.57 (s, 1H), 7.83 (dd, J = 11.3, 1.9 Hz, 1H), 7.69 (d, J = 8.6 |
| | Hz, 2H), 7.39 (d, J = 8.6 Hz, 2H), 7.35 (d, J = 1.8 Hz, 1H), 7.09-6.99 (m, 2H), 5.48-5.37 (m, |
| | 1H), 3.91-3.81 (m, 1H), 3.75-3.61 (m, 1H), 2.52-2.38 (m, 1H), 2.21-1.98 (m, 3H). |

### Example 220: (S)-5-(4-(1-((4-isopropylphenyl)carbamoyl)-3,3-dimethylazetidine-2-carboxamido)phenyl)picolinic acid and (R)-5-(4-(1-((4-isopropylphenyl)carbamoyl)-3,3-dimethylazetidine-2-carboxamido)phenyl)picolinic acid

### Step 1: Methyl 5-(4-aminophenyl)picolinate

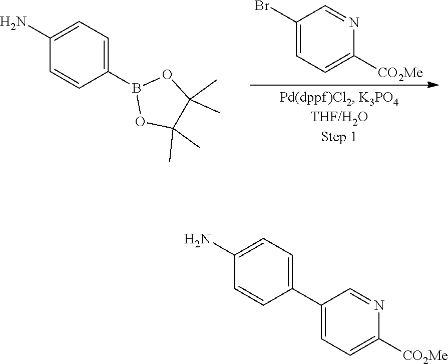 [SMILES: CC1(C)OB(c2ccc(N)cc2)OC1(C)C ; COC(=O)c1ccc(Br)cn1 ; COC(=O)c1ccc(-c2ccc(N)cc2)cn1]

A solution of 4-(4,4,5,5-tetramethyl-1,3,2-dioxaborolan-2-yl)aniline (200 mg, 0.913 mmol), methyl 5-bromopyridine-2-carboxylate (237 mg, 1.10 mmol), Pd(dppf)Cl₂ (66.8 mg, 0.091 mmol), and tribasic potassium phosphate (581 mg, 2.74 mmol) in THF (2.0 mL) and water (0.20 mL) was stirred at 80° C. for 1 hour. Upon completion, the mixture was concentrated under reduced pressure. The crude residue was directly purified by silica gel column chromatography, eluted with petroleum ether/ethyl acetate (1:1) to afford methyl 5-(4-aminophenyl)picolinate (150 mg, 71%) as a yellow solid. LCMS (ESI) [M+H]⁺: 229.0.

### Step 2: methyl 5-(4-(1-((4-isopropylphenyl)carbamoyl)-3,3-dimethylazetidine-2-carboxamido)phenyl)picolinate

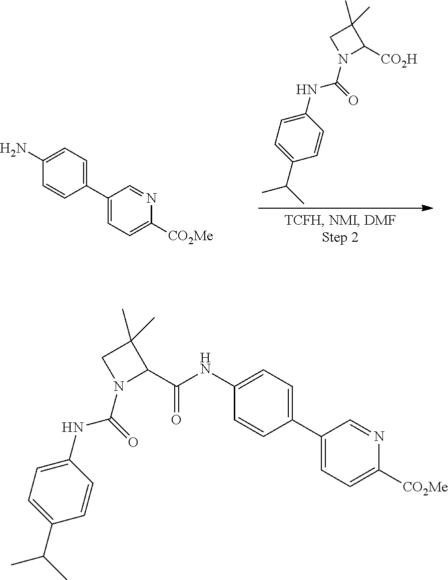 [SMILES: COC(=O)c1ccc(-c2ccc(N)cc2)cn1 ; CC(C)c1ccc(NC(=O)N2CC(C)(C)C2C(=O)O)cc1 ; COC(=O)c1ccc(-c2ccc(NC(=O)C3N(C(=O)Nc4ccc(C(C)C)cc4)CC3(C)C)cc2)cn1]

A solution of methyl 5-(4-aminophenyl)picolinate (150 mg, 0.657 mmol), 1-methylimidazole (89.9 mg, 1.09 mmol), TCFH (230 mg, 0.821 mmol), and 1-((4-isopropylphenyl)carbamoyl)-3,3-dimethylazetidine-2-carboxylic acid (159 mg, 0.548 mmol) in DMF (1.0 mL) was stirred at 25° C. for 1 hour. Upon completion, the reaction mixture was directly purified by reversed-phase flash chromatography (conditions: column: C18 silica gel; mobile phase A: water (0.1% formic acid); mobile phase B: MeCN; gradient (B %): 0% to 100% over 30 min; detector: UV 254 nm) to afford methyl 5-(4-(1-((4-isopropylphenyl)carbamoyl)-3,3-dimethylazetidine-2-carboxamido)phenyl)picolinate (100 mg, 36%) as a white solid. LCMS (ESI) [M+H]⁺: 501.2.

### Step 3: 5-(4-(1-((4-isopropylphenyl)carbamoyl)-3,3-dimethylazetidine-2-carboxamido)phenyl)picolinic acid

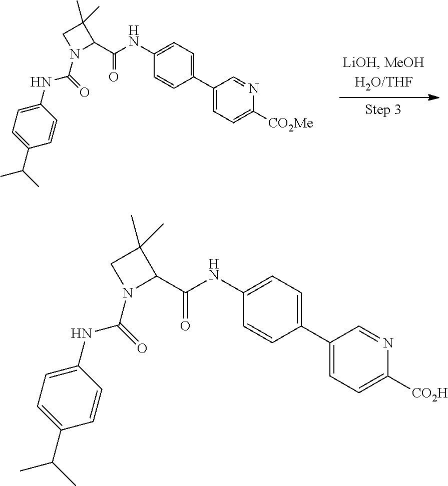 [SMILES: COC(=O)c1ccc(-c2ccc(NC(=O)C3N(C(=O)Nc4ccc(C(C)C)cc4)CC3(C)C)cc2)cn1 ; CC(C)c1ccc(NC(=O)N2CC(C)(C)C2C(=O)Nc2ccc(-c3ccc(C(=O)O)nc3)cc2)cc1]

A solution of methyl 5-(4-(1-((4-isopropylphenyl)carbamoyl)-3,3-dimethylazetidine-2-carboxamido)phenyl)picolinate (80.0 mg, 0.160 mmol) and lithium hydroxide monohydrate (13.5 mg, 0.320 mmol) in methanol (1.0 mL), THF (1.0 mL) and water (1.0 mL) was stirred at 25° C. for 1 hour. Upon completion, the reaction mixture was directly purified by reversed-phase flash chromatography (conditions: column: C18 silica gel; mobile phase A: water (0.1% formic acid); mobile phase B: MeCN; gradient (B %): 0% to 100% over 30 min; detector: UV 254 nm) to afford 5-(4-(1-((4-isopropylphenyl)carbamoyl)-3,3-dimethylazetidine-2-carboxamido)phenyl)picolinic acid (40 mg, 51%) as a white solid. LCMS (ESI) [M+H]⁺: 487.2.

### Step 4: (S)-5-(4-(1-((4-isopropylphenyl)carbamoyl)-3,3-dimethylazetidine-2-carboxamido)phenyl)picolinic acid and (R)-5-(4-(1-((4-isopropylphenyl)carbamoyl)-3,3-dimethylazetidine-2-carboxamido)phenyl)picolinic acid

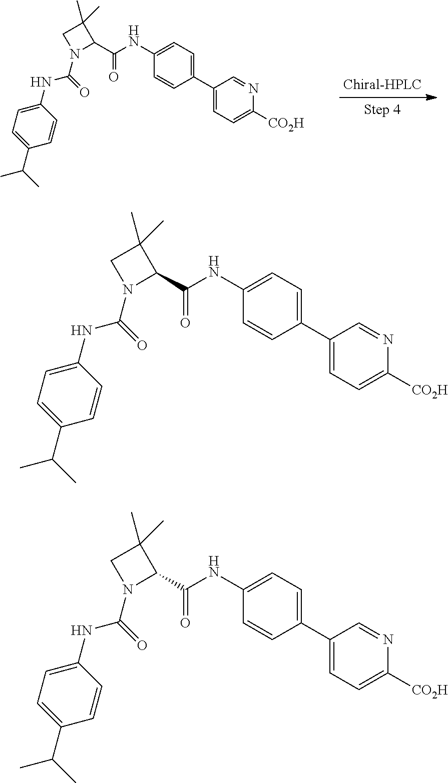 [SMILES: CC(C)c1ccc(NC(=O)N2CC(C)(C)C2C(=O)Nc2ccc(-c3ccc(C(=O)O)nc3)cc2)cc1 ; CC(C)c1ccc(NC(=O)N2CC(C)(C)[C@H]2C(=O)Nc2ccc(-c3ccc(C(=O)O)nc3)cc2)cc1 ; CC(C)c1ccc(NC(=O)N2CC(C)(C)[C@@H]2C(=O)Nc2ccc(-c3ccc(C(=O)O)nc3)cc2)cc1]

5-(4-(1-((4-isopropylphenyl)carbamoyl)-3,3-dimethylazetidine-2-carboxamido)phenyl)picolinic acid (40.0 mg) was resolved by preparative chiral HPLC (conditions: column: CHIRALPAK IK 3*25 cm, 5 μm; mobile phase A: MTBE (0.1% trifluoroacetic acid), mobile phase B: methanol; flow rate: 40 mL/min; gradient (B %): isocratic, 50% B; detector: 220/254 nm; RT1 (min): 6.3; RT2 (min): 28.4) to afford (Compound 220a) (S)-5-(4-(1-((4-isopropylphenyl)carbamoyl)-3,3-dimethylazetidine-2-carboxamido)phenyl)picolinic acid (1^{st} peak, 13.8 mg, 34%; Method BH, Peak 1, 0.84 min) as a white solid, and (Compound 220b) (R)-5-(4-(1-((4-isopropylphenyl)carbamoyl)-3,3-dimethylazetidine-2-carboxamido)phenyl)picolinic acid (2^{nd} peak, 11.3 mg, 28%; Method BH, Peak 2, 3.41 min) as a white solid.

| Cmpd # | Characterization Data |
|---|---|
| 220a | LCMS (ESI) [M + H]⁺: 487.2 |
| | ¹H NMR (400 MHz, methanol-d₄) δ 8.94 (s, 1H), 8.34-8.22 (m, 2H), 7.83 (d, J = 8.0 Hz, 2H), 7.75 |
| | (d, J = 8.2 Hz, 2H), 7.36 (d, J = 8.1 Hz, 2H), 7.14 (d, J = 7.9 Hz, 2H), 4.49 (s, 1H), 3.85 (d, J = 7.3 |
| | Hz, 1H), 3.78 (d, J = 7.2 Hz, 1H), 2.85 (p, J = 7.1 Hz, 1H), 1.47 (s, 3H), 1.27 (s, 3H), 1.23 (s, 3H), |
| | 1.21 (s, 3H). |
| 220b | LCMS (ESI) [M + H]⁺: 487.2 |
| | ¹H NMR (400 MHz, methanol-d₄) δ 8.94 (s, 1H), 8.27 (d, J = 14.2 Hz, 2H), 7.83 (d, J = 7.9 Hz, |
| | 2H), 7.75 (d, J = 8.0 Hz, 2H), 7.36 (d, J = 8.1 Hz, 2H), 7.14 (d, J = 7.9 Hz, 2H), 4.49 (s, 1H), 3.85 |
| | (d, J = 7.2 Hz, 1H), 3.78 (d, J = 7.2 Hz, 1H), 2.89-2.82 (m, 1H), 1.47 (s, 3H), 1.27 (s, 3H), 1.23 (s, |
| | 3H), 1.21 (s, 3H). |

### Example 221: (S)-4-(6-(1-((4-isopropylphenyl)carbamoyl)-3,3-dimethylazetidine-2-carboxamido)pyridin-3-yl)benzoic acid and (R)-4-(6-(1-((4-isopropylphenyl)carbamoyl)-3,3-dimethylazetidine-2-carboxamido)pyridin-3-yl)benzoic acid

### Step 1: Methyl 4-(6-aminopyridin-3-yl)benzoate

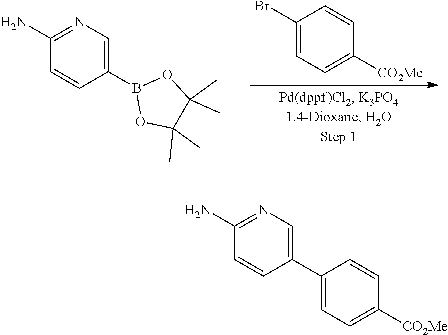 [SMILES: CC1(C)OB(c2ccc(N)nc2)OC1(C)C ; COC(=O)c1ccc(Br)cc1 ; COC(=O)c1ccc(-c2ccc(N)nc2)cc1]

A solution of methyl 4-bromobenzoate (300 mg, 1.39 mmol), 5-(4,4,5,5-tetramethyl-1,3,2-dioxaborolan-2-yl)pyridin-2-amine (461 mg, 2.09 mmol), Pd(dppf)Cl₂ (114 mg, 0.140 mmol), and tribasic potassium phosphate (888 mg, 4.18 mmol) in 1,4-dioxane (4.0 mL) and water (1.0 mL) was heated to 80° C. for 2 hours. Upon completion, the reaction mixture was concentrated under reduced pressure. The crude residue was directly purified by silica gel chromatography, eluted with petroleum ether/ethyl acetate (20:1), to afford methyl 4-(6-aminopyridin-3-yl)benzoate (280 mg, 88%) as a brown solid. LCMS (ESI) [M+H]⁺: 229.

### Step 2: methyl 4-(6-(1-((4-isopropylphenyl)carbamoyl)-3,3-dimethylazetidine-2-carboxamido)pyridin-3-yl)benzoate

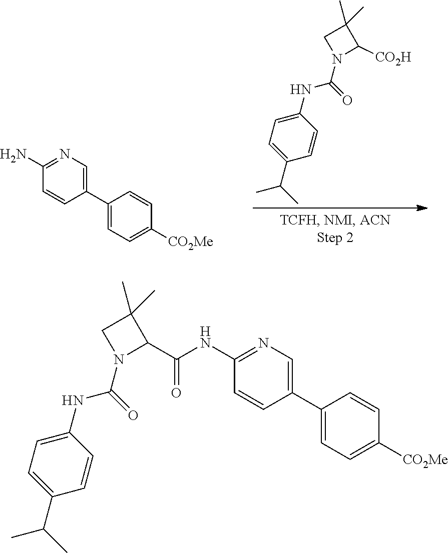

A solution of 1-((4-isopropylphenyl)carbamoyl)-3,3-dimethylazetidine-2-carboxylic acid (50.0 mg, 0.172 mmol), 1-methylimidazole (84.8 mg, 1.03 mmol), TCFH (96.6 mg, 0.344 mmol) and methyl 4-(6-aminopyridin-3-yl)benzoate (47.2 mg, 0.206 mmol) in MeCN (2.0 mL) was stirred at room temperature for 2 hours. Upon completion, the reaction mixture was concentrated under reduced pressure. The crude residue was directly purified by silica gel column chromatography, eluted with petroleum ether/ethyl acetate (5:1) to afford methyl 4-(6-(1-((4-isopropylphenyl)carbamoyl)-3,3-dimethylazetidine-2-carboxamido)pyridin-3-yl)benzoate (60.0 mg, 69%) as a tan solid. LCMS (ESI) [M+H]⁺: 501.6.

### Step 3: 4-(6-(1-((4-isopropylphenyl)carbamoyl)-3,3-dimethylazetidine-2-carboxamido)pyridin-3-yl)benzoic acid

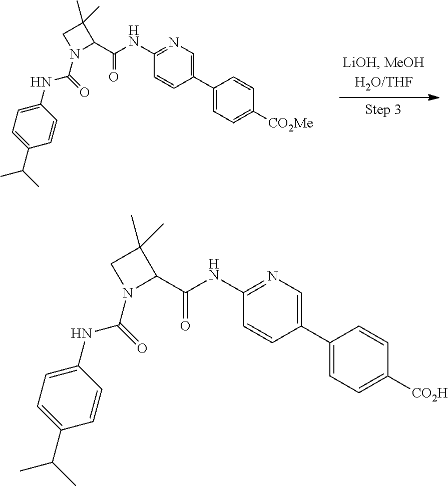 [SMILES: COC(=O)c1ccc(-c2ccc(NC(=O)C3N(C(=O)Nc4ccc(C(C)C)cc4)CC3(C)C)nc2)cc1 ; CC(C)c1ccc(NC(=O)N2CC(C)(C)C2C(=O)Nc2ccc(-c3ccc(C(=O)O)cc3)cn2)cc1]

To methyl 4-(6-(1-((4-isopropylphenyl)carbamoyl)-3,3-dimethylazetidine-2-carboxamido)pyridin-3-yl)benzoate (55.0 mg, 0.110 mmol) in a solution of methanol (1.0 mL), THF (1.0 mL), and water (1.0 mL) was added lithium hydroxide monohydrate (23.1 mg, 0.550 mmol) at room temperature. After 2 hours, the reaction mixture was concentrated under reduced pressure. The crude residue was directly purified by reversed-phase column chromatography (conditions: column: C18 silica gel; mobile phase A: water (0.1% formic acid); mobile phase B: MeCN; gradient (B %): 0% to 100% over 20 min; detector: UV 254 nm) to give 4-(6-(1-((4-isopropylphenyl)carbamoyl)-3,3-dimethylazetidine-2-carboxamido)pyridin-3-yl)benzoic acid (32.0 mg, 60%) as an off-white solid. LCMS (ESI) [M+H]⁺: 487.6.

### Step 4: (S)-4-(6-(1-((4-isopropylphenyl)carbamoyl)-3,3-dimethylazetidine-2-carboxamido)pyridin-3-yl)benzoic acid and (R)-4-(6-(1-((4-isopropylphenyl)carbamoyl)-3,3-dimethylazetidine-2-carboxamido)pyridin-3-yl)benzoic acid

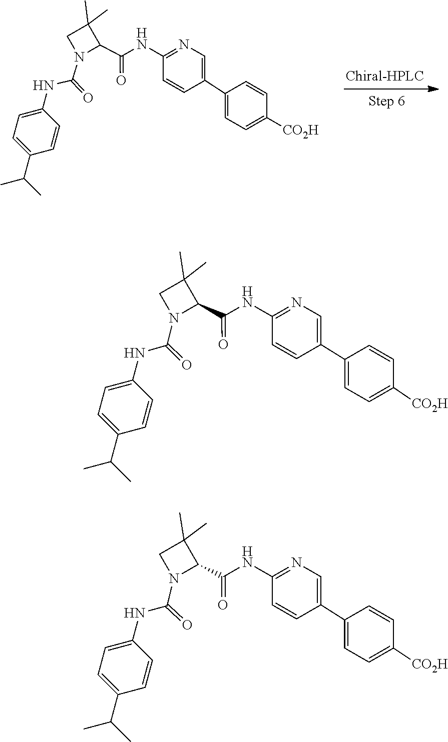 [SMILES: CC(C)c1ccc(NC(=O)N2CC(C)(C)C2C(=O)Nc2ccc(-c3ccc(C(=O)O)cc3)cn2)cc1 ; CC(C)c1ccc(NC(=O)N2CC(C)(C)[C@H]2C(=O)Nc2ccc(-c3ccc(C(=O)O)cc3)cn2)cc1 ; CC(C)c1ccc(NC(=O)N2CC(C)(C)[C@@H]2C(=O)Nc2ccc(-c3ccc(C(=O)O)cc3)cn2)cc1]

4-(6-(1-((4-isopropylphenyl)carbamoyl)-3,3-dimethylazetidine-2-carboxamido)pyridin-3-yl)benzoic acid (32 mg) was resolved by preparative chiral HPLC (conditions: Column: CHIRAL ART Cellulose-SB, 3*25 cm, 5 μm; mobile phase A: hexane (0.1% formic acid) mobile phase B: ethanol; flow rate: 40 mL/min; gradient (B %): isocratic, 50% B; detector: 220/254 nm; RT1 (min): 6.4; RT2 (min): 11.5) to afford (Compound 221a) (S)-4-(6-(1-((4-isopropylphenyl)carbamoyl)-3,3-dimethylazetidine-2-carboxamido)pyridin-3-yl)benzoic acid (1^{st} peak, 8.7 mg, 29%; Method BI, Peak 1, 0.66 min) as a white solid, and (Compound 221b) (R)-4-(6-(1-((4-isopropylphenyl)carbamoyl)-3,3-dimethylazetidine-2-carboxamido)pyridin-3-yl)benzoic acid (2^{nd} peak, 7.3 mg, 24%; Method BI, Peak 2, 1.17 min) as a white solid.

| Cmpd # | Characterization Data |
|---|---|
| 221a | LCMS (ESI) [M + H]⁺: 487.3 |
| | ¹H NMR (400 MHz, methanol-d₄) δ 8.65 (d, J = 2.5 Hz, 1H), 8.32 (d, J = 8.7 Hz, 1H), 8.13 (dd, J = |
| | 11.0, 8.3 Hz, 3H), 7.76 (d, J = 8.1 Hz, 2H), 7.37 (d, J = 8.6 Hz, 2H), 7.14 (d, J = 8.5 Hz, 2H), |
| | 4.54 (s, 1H), 3.85 (d, J = 7.4 Hz, 1H), 3.80 (d, J = 7.4 Hz, 1H), 2.85 (p, J = 6.9 Hz, 1H), 1.47 (s, |
| | 3H), 1.26 (s, 3H), 1.23 (s, 3H), 1.21 (s, 3H). |
| 221b | LCMS (ESI) [M + H]⁺: 487.3 |
| | ¹H NMR (400 MHz, methanol- d₄) δ 8.65 (d, J = 2.4 Hz, 1H), 8.32 (d, J = 8.7 Hz, 1H), 8.19-8.05 |
| | (m, 3H), 7.77 (d, J = 8.2 Hz, 2H), 7.37 (d, J = 8.6 Hz, 2H), 7.14 (d, J = 8.6 Hz, 2H), 4.54 (s, 1H), |
| | 3.85 (d, J = 7.3 Hz, 1H), 3.80 (d, J = 7.4 Hz, 1H), 2.85 (p, J = 6.9 Hz, 1H), 1.47 (s, 3H), 1.26 (s, |
| | 3H), 1.23 (s, 3H), 1.21 (s, 3H). |

### Example 222: (S)-3′-fluoro-4′-(1-((4-isopropylphenyl)carbamoyl)-3,3-dimethylazetidine-2-carboxamido)-[1,1′-biphenyl]-4-carboxylic acid and (R)-3′-fluoro-4′-(1-((4-isopropylphenyl)carbamoyl)-3,3-dimethylazetidine-2-carboxamido)-[1,1′-biphenyl]-4-carboxylic acid

### Step 1: methyl 4′-amino-3′-fluoro-[1,1′-biphenyl]-4-carboxylate

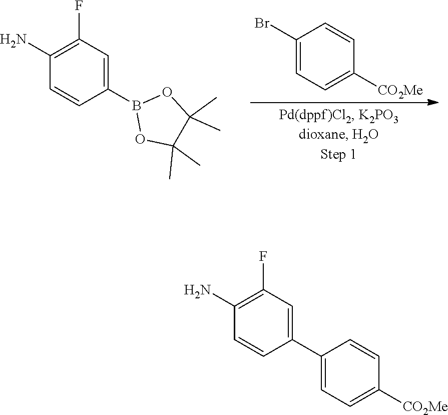 [SMILES: CC1(C)OB(c2ccc(N)c(F)c2)OC1(C)C ; COC(=O)c1ccc(Br)cc1 ; COC(=O)c1ccc(-c2ccc(N)c(F)c2)cc1]

To a solution of methyl 4-bromobenzoate (453 mg, 2.10 mmol), 2-fluoro-4-(4,4,5,5-tetramethyl-1,3,2-dioxaborolan-2-yl)aniline (500 mg, 2.11 mmol) and Pd(dppf)Cl₂ (154 mg, 0.210 mmol) in 1,4-dioxane (5.0 mL) was added a solution of K₂CO₃ (874 mg, 6.32 mmol) in water (1.0 mL), and the resultant mixture was stirred at 80° C. for 2 hours. Upon completion, the reaction mixture was diluted with water (20 mL) and extracted with ethyl acetate (3×20 mL). The combined organic layers were concentrated under reduced pressure. The crude residue was purified by silica gel column chromatography, eluted with petroleum ether/ethyl acetate (1:1) to afford methyl 4′-amino-3′-fluoro-[1,1′-biphenyl]-4-carboxylate (400 mg, 77%) as a white solid. LCMS (ESI) [M+H]⁺: 246.0.

### Step 2: methyl 3′-fluoro-4′-(1-((4-isopropylphenyl)carbamoyl)-3,3-dimethylazetidine-2-carboxamido)-[1,1′-biphenyl]-4-carboxylate

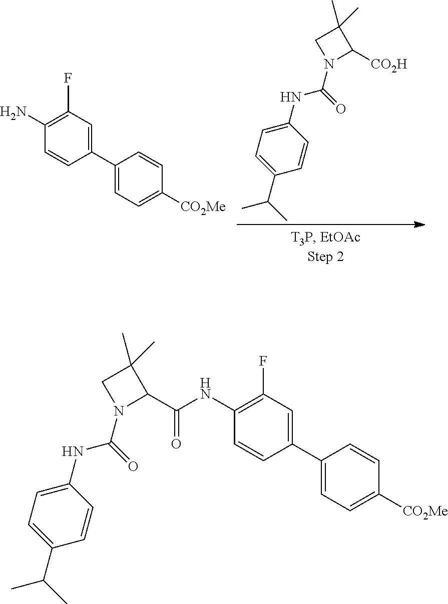 [SMILES: COC(=O)c1ccc(-c2ccc(N)c(F)c2)cc1 ; CC(C)c1ccc(NC(=O)N2CC(C)(C)C2C(=O)O)cc1 ; COC(=O)c1ccc(-c2ccc(NC(=O)C3N(C(=O)Nc4ccc(C(C)C)cc4)CC3(C)C)c(F)c2)cc1]

A solution of propanephosphonic acid cyclic anhydride (0.36 mL, 0.47 mmol, 50% in ethyl acetate) was added to a solution of methyl 4′-amino-3′-fluoro-[1,1′-biphenyl]-4-carboxylate (100 mg, 0.400 mmol) and 1-[(4-isopropylphenyl)carbamoyl]-3,3-dimethylazetidine-2-carboxylic acid (118 mg, 0.400 mmol) in ethyl acetate (2.0 mL). The resultant mixture was heated to 50° C. for 1 hour. Upon completion, the mixture was concentrated under reduced pressure. The crude residue was directly purified by silica gel column chromatography, eluted with petroleum ether/ethyl acetate (1:2), to afford methyl 3′-fluoro-4′-(1-((4-isopropylphenyl)carbamoyl)-3,3-dimethylazetidine-2-carboxamido)-[1,1′-biphenyl]-4-carboxylate (70.0 mg, 33%) as a white solid. LCMS (ESI) [M+H]⁺: 518.0.

### Step 3: 3′-fluoro-4′-(1-((4-isopropylphenyl)carbamoyl)-3,3-dimethylazetidine-2-carboxamido)-[1,1′-biphenyl]-4-carboxylic acid

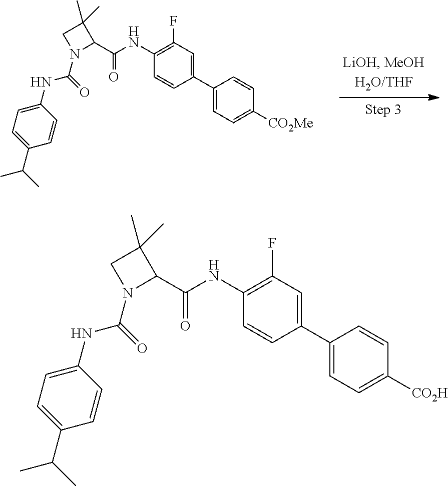 [SMILES: COC(=O)c1ccc(-c2ccc(NC(=O)C3N(C(=O)Nc4ccc(C(C)C)cc4)CC3(C)C)c(F)c2)cc1 ; CC(C)c1ccc(NC(=O)N2CC(C)(C)C2C(=O)Nc2ccc(-c3ccc(C(=O)O)cc3)cc2F)cc1]

To a mixture of methyl 3′-fluoro-4′-(1-((4-isopropylphenyl)carbamoyl)-3,3-dimethylazetidine-2-carboxamido)-[1,1′-biphenyl]-4-carboxylate (60.0 mg, 0.110 mmol), in methanol (1.0 mL), THF (1.0 mL), and water (1.0 mL) was added lithium hydroxide monohydrate (19.3 mg, 0.460 mmol) at room temperature. After 2 hours, the reaction mixture was concentrated under reduced pressure. The crude residue was directly purified by reversed phase chromatography (conditions: column: C18 silica gel; mobile phase A: water (0.1% formic acid); mobile phase B: MeCN; gradient (B %): 0% to 100% over 20 min; detector: UV 254 nm) to give 3′-fluoro-4′-(1-((4-isopropylphenyl)carbamoyl)-3,3-dimethylazetidine-2-carboxamido)-[1,1′-biphenyl]-4-carboxylic acid (45 mg, 77%) as an off-white solid. LCMS (ESI) [M+H]⁺: 504.0.

### Step 4: (S)-3′-fluoro-4′-(1-((4-isopropylphenyl)carbamoyl)-3,3-dimethylazetidine-2-carboxamido)-[1,1′-biphenyl]-4-carboxylic acid and (R)-3′-fluoro-4′-(1-((4-isopropylphenyl)carbamoyl)-3,3-dimethylazetidine-2-carboxamido)-[1,1′-biphenyl]-4-carboxylic acid

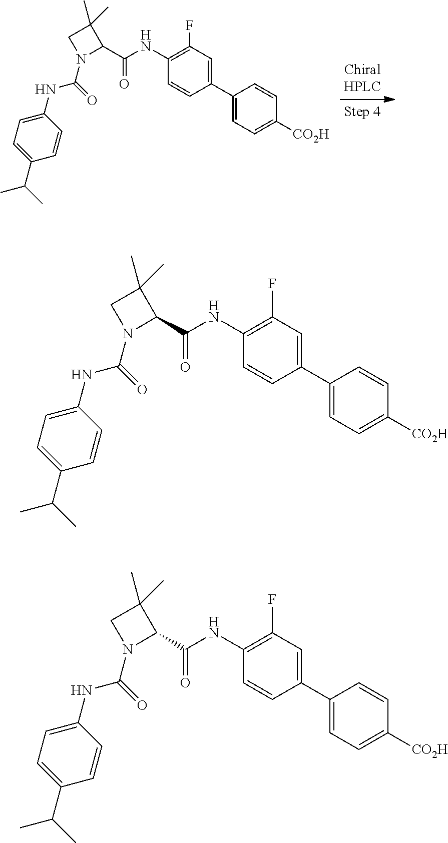 [SMILES: CC(C)c1ccc(NC(=O)N2CC(C)(C)C2C(=O)Nc2ccc(-c3ccc(C(=O)O)cc3)cc2F)cc1 ; CC(C)c1ccc(NC(=O)N2CC(C)(C)[C@H]2C(=O)Nc2ccc(-c3ccc(C(=O)O)cc3)cc2F)cc1 ; CC(C)c1ccc(NC(=O)N2CC(C)(C)[C@@H]2C(=O)Nc2ccc(-c3ccc(C(=O)O)cc3)cc2F)cc1]

3′-fluoro-4′-(1-((4-isopropylphenyl)carbamoyl)-3,3-dimethylazetidine-2-carboxamido)-[1,1′-biphenyl]-4-carboxylic acid (45.0 mg) was resolved by preparative chiral HPLC (conditions: column: CHIRAL ART Cellulose-SZ, 3*25 cm, 5 μm; mobile phase A: MTBE (0.1% formic acid), mobile phase B: methanol; flow rate: 40 mL/min; gradient (B %): isocratic, 30% B; detector: 220/254 nm; RT1 (min): 5.5; RT2 (min): 9.7) to afford (Compound 222a) (S)-3′-fluoro-4′-(1-((4-isopropylphenyl)carbamoyl)-3,3-dimethylazetidine-2-carboxamido)-[1,1′-biphenyl]-4-carboxylic acid (1^{st} peak, 13.9 mg, 31%; Method BJ, Peak 1, 0.63 min) as a white solid and (Compound 222b) (R)-3′-fluoro-4′-(1-((4-isopropylphenyl)carbamoyl)-3,3-dimethylazetidine-2-carboxamido)-[1,1′-biphenyl]-4-carboxylic acid (2^{nd} peak, 11.9 mg, 26%; Method BJ, Peak 2, 1.29 min) as a white solid.

| Cmpd # | Characterization Data |
|---|---|
| 222a | LCMS (ESI) [M + H]⁺: 504.3 |
| | ¹H NMR (400 MHz, methanol-d₄) δ 8.12-8.00 (m, 3H), 7.74 (dd, J = 8.4, 2.2 Hz, 2H), 7.53 (td, |
| | J = 8.2, 6.9, 3.0 Hz, 2H), 7.38-7.32 (m, 2H), 7.15 (d, J = 8.4 Hz, 2H), 4.56 (s, 1H), 3.87-3.76 |
| | (m, 2H), 2.85 (p, J = 6.9 Hz, 1H), 1.46 (s, 3H), 1.30 (s, 3H), 1.23 (s, 3H), 1.21 (s, 3H). |
| 222b | LCMS (ESI) [M + H]⁺: 504.3 |
| | ¹H NMR (400 MHz, methanol-d₄) δ 8.12-8.01 (m, 3H), 7.74 (d, J = 8.5 Hz, 2H), 7.58-7.49 |
| | (m, 2H), 7.36 (d, J = 8.5 Hz, 2H), 7.15 (d, J = 8.5 Hz, 2H), 4.55 (s, 1H), 3.87-3.75 (m, 2H), |
| | 2.85 (p, J = 6.9 Hz, 1H), 1.46 (s, 3H), 1.30 (s, 3H), 1.23 (s, 3H), 1.21 (s, 3H). |

### Example 223: (S)-4′-(1-((4-isopropylphenyl)carbamoyl)-3,3-dimethylazetidine-2-carboxamido)-3-methoxy-[1,1′-biphenyl]-4-carboxylic acid and (R)-4′-(1-((4-isopropylphenyl)carbamoyl)-3,3-dimethylazetidine-2-carboxamido)-3-methoxy-[1,1′-biphenyl]-4-carboxylic acid

### Step 1: methyl 4′-amino-3-methoxy-[1,1′-biphenyl]-4-carboxylate

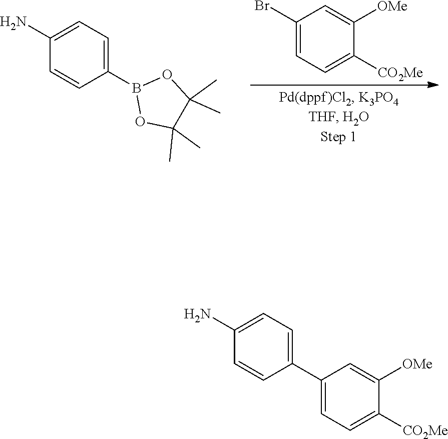 [SMILES: CC1(C)OB(c2ccc(N)cc2)OC1(C)C ; COC(=O)c1ccc(Br)cc1OC ; COC(=O)c1ccc(-c2ccc(N)cc2)cc1OC]

A solution of 4-(4,4,5,5-tetramethyl-1,3,2-dioxaborolan-2-yl)aniline (200 mg, 0.913 mmol), methyl 4-bromo-2-methoxybenzoate (268 mg, 1.09 mmol), Pd(dppf)Cl₂ (66.8 mg, 0.091 mmol) and tribasic potassium phosphate (581 mg, 2.74 mmol) in a solution of THF (2.0 mL) and water (0.20 mL) was heated to at 80° C. for 1 hour. Upon completion, the mixture was concentrated under reduced pressure. The crude residue was directly purified by silica gel column chromatography, eluted with petroleum ether/ethyl acetate (1:1) to afford methyl 4′-amino-3-methoxy-[1,1′-biphenyl]-4-carboxylate (150 mg, 63%) as a yellow solid. LCMS (ESI) [M+H]⁺: 258.1.

### Step 2: methyl 4′-(1-((4-isopropylphenyl)carbamoyl)-3,3-dimethylazetidine-2-carboxamido)-3-methoxy-[1,1′-biphenyl]-4-carboxylate

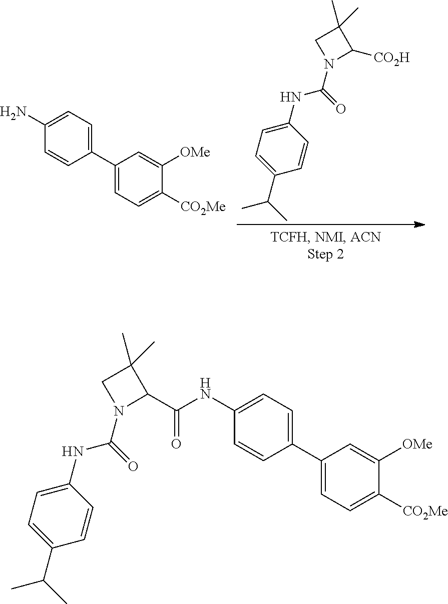 [SMILES: COC(=O)c1ccc(-c2ccc(N)cc2)cc1OC ; CC(C)c1ccc(NC(=O)N2CC(C)(C)C2C(=O)O)cc1 ; COC(=O)c1ccc(-c2ccc(NC(=O)C3N(C(=O)Nc4ccc(C(C)C)cc4)CC3(C)C)cc2)cc1OC]

A solution of methyl 4′-amino-3-methoxy-[1,1′-biphenyl]-4-carboxylate (150 mg, 0.583 mmol), (79.8 mg, 0.972 mmol), TCFH (204 mg, 0.729 mmol), and 1-((4-isopropylphenyl)carbamoyl)-3,3-dimethylazetidine-2-carboxylic acid (141 mg, 0.486 mmol) in DMF (2.0 mL) was stirred at 25° C. for 1 hour. Upon completion, the mixture was concentrated under reduced pressure. The crude residue was directly purified by reversed phase chromatography (conditions: column: C18 silica gel; mobile phase A: water (0.1% formic acid); mobile phase B: MeCN; gradient (B %): 0% to 100% over 20 min; detector: UV 254 nm) to afford methyl 4′-(1-((4-isopropylphenyl)carbamoyl)-3,3-dimethylazetidine-2-carboxamido)-3-methoxy-[1,1′-biphenyl]-4-carboxylate (100 mg, 38%) as a yellow solid. LCMS (ESI) [M+H]⁺: 530.2.

### Step 3: 4′-(1-((4-isopropylphenyl)carbamoyl)-3,3-dimethylazetidine-2-carboxamido)-3-methoxy-[1,1′-biphenyl]-4-carboxylic acid

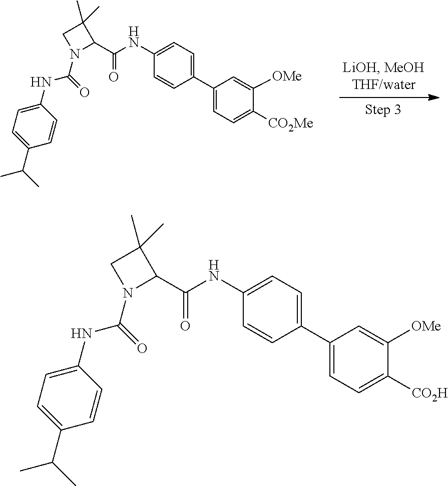 [SMILES: COC(=O)c1ccc(-c2ccc(NC(=O)C3N(C(=O)Nc4ccc(C(C)C)cc4)CC3(C)C)cc2)cc1OC ; COc1cc(-c2ccc(NC(=O)C3N(C(=O)Nc4ccc(C(C)C)cc4)CC3(C)C)cc2)ccc1C(=O)O]

A solution of methyl 4′-(1-((4-isopropylphenyl)carbamoyl)-3,3-dimethylazetidine-2-carboxamido)-3-methoxy-[1,1′-biphenyl]-4-carboxylate (80 mg, 0.151 mmol) and lithium hydroxide monohydrate (12.7 mg, 0.302 mmol) in methanol (1.0 mL), THF (1.0 mL) and water (1.0 mL) was stirred at 25° C. for 1 hour. Upon completion, the reaction mixture was concentrated under reduced pressure. The crude residue was directly purified by reversed phase chromatography (conditions: column: C18 silica gel; mobile phase A: water (0.1% formic acid); mobile phase B: MeCN; gradient (B %): 0% to 100% over 20 min; detector: UV 254 nm) to afford 4′-(1-((4-isopropylphenyl)carbamoyl)-3,3-dimethylazetidine-2-carboxamido)-3-methoxy-[1,1′-biphenyl]-4-carboxylic acid (30 mg, 38%) as a white solid. LCMS (ESI) [M+H]⁺: 516.2.

### Step 4: (S)-4′-(1-((4-isopropylphenyl)carbamoyl)-3,3-dimethylazetidine-2-carboxamido)-3-methoxy-[1,1′-biphenyl]-4-carboxylic acid and (R)-4′-(1-((4-isopropylphenyl)carbamoyl)-3,3-dimethylazetidine-2-carboxamido)-3-methoxy-[1,1′-biphenyl]-4-carboxylic acid

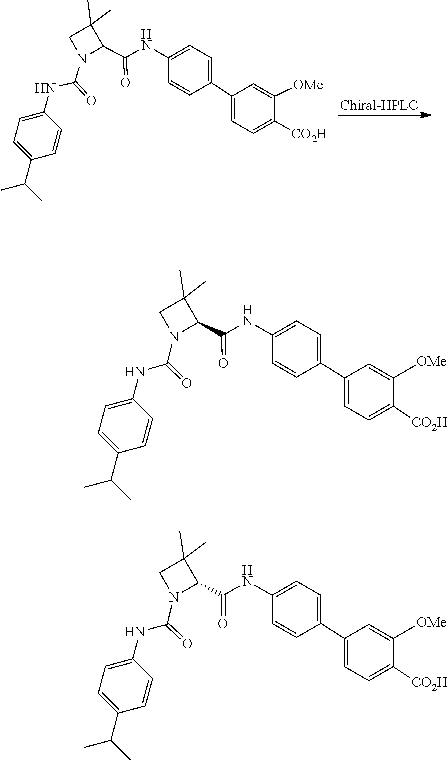 [SMILES: COc1cc(-c2ccc(NC(=O)C3N(C(=O)Nc4ccc(C(C)C)cc4)CC3(C)C)cc2)ccc1C(=O)O ; COc1cc(-c2ccc(NC(=O)[C@H]3N(C(=O)Nc4ccc(C(C)C)cc4)CC3(C)C)cc2)ccc1C(=O)O ; COc1cc(-c2ccc(NC(=O)[C@@H]3N(C(=O)Nc4ccc(C(C)C)cc4)CC3(C)C)cc2)ccc1C(=O)O]

4′-(1-((4-isopropylphenyl)carbamoyl)-3,3-dimethylazetidine-2-carboxamido)-3-methoxy-[1,1′-biphenyl]-4-carboxylic acid (30 mg) was resolved by preparative chiral HPLC (conditions: Column: CHIRAL ART Cellulose-SZ, 3*25 cm, 5 μm; mobile phase A: MTBE (0.1% formic acid), mobile phase B: methanol; flow rate: 40 mL/min; gradient (B %): isocratic, 50% B; detector: 220/254 nm; RT1 (min): 6; RT2 (min): 11.7) to afford (Compound 223a) (S)-4′-(1-((4-isopropylphenyl)carbamoyl)-3,3-dimethylazetidine-2-carboxamido)-3-methoxy-[1,1′-biphenyl]-4-carboxylic acid (1^{st} peak, 7.6 mg, 25%; Method BK, Peak 1, 0.59 min) as a white solid and (Compound 223b) (R)-4′-(1-((4-isopropylphenyl)carbamoyl)-3,3-dimethylazetidine-2-carboxamido)-3-methoxy-[1,1′-biphenyl]-4-carboxylic acid (2^{nd} peak, 7.3 mg, 24%; Method BK, Peak 2, 0.97 min) as a white solid.

| Cmpd # | Characterization Data |
|---|---|
| 223a | LCMS (ESI) [M + H]⁺: 516.4 |
| | ¹H NMR (400 MHz, methanol-d₄) δ 7.89 (d, J = 8.1 Hz, 1H), 7.74 (d, J = 8.8 Hz, 2H), 7.68 (d, J = |
| | 8.8 Hz, 2H), 7.38-7.31 (m, 3H), 7.28 (dd, J = 8.1, 1.6 Hz, 1H), 7.14 (d, J = 8.5 Hz, 2H), 4.49 (s, |
| | 1H), 3.99 (s, 3H), 3.85 (d, J = 7.3 Hz, 1H), 3.78 (d, J = 7.3 Hz, 1H), 2.85 (p, J = 6.9 Hz, 1H), 1.47 |
| | (s, 3H), 1.27 (s, 3H), 1.23 (s, 3H), 1.21 (s, 3H). |
| 223b | LCMS (ESI) [M + H]⁺: 516.3 |
| | ¹H NMR (400 MHz, methanol-d₄) δ 7.88 (d, J = 8.2 Hz, 1H), 7.75 (d, J = 8.9 Hz, 2H), 7.68 (d, J = |
| | 8.5 Hz, 2H), 7.35 (dd, J = 9.7, 8.0 Hz, 3H), 7.28 (d, J = 8.1 Hz, 1H), 7.15 (d, J = 8.3 Hz, 2H), 4.49 |
| | (d, J = 1.6 Hz, 1H), 3.99 (s, 3H), 3.85 (d, J = 7.4 Hz, 1H), 3.78 (d, J = 7.3 Hz, 1H), 2.85 (p, J = |
| | 6.8 Hz, 1H), 1.47 (s, 3H), 1.27 (s, 3H), 1.23 (s, 3H), 1.21 (s, 3H). |

### Example 224: (S)-4-(5-(1-((4-isopropylphenyl)carbamoyl)-3,3-dimethylazetidine-2-carboxamido)pyridin-2-yl)benzoic acid and (R)-4-(5-(1-((4-isopropylphenyl)carbamoyl)-3,3-dimethylazetidine-2-carboxamido)pyridin-2-yl)benzoic acid

### Step 1: methyl 4-(5-aminopyridin-2-yl)benzoate

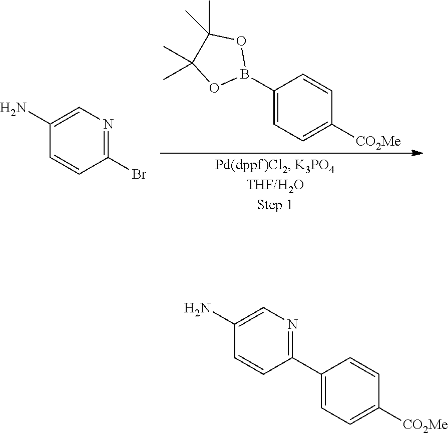 [SMILES: Nc1ccc(Br)nc1 ; COC(=O)c1ccc(B2OC(C)(C)C(C)(C)O2)cc1 ; COC(=O)c1ccc(-c2ccc(N)cn2)cc1]

A stirred mixture of methyl 4-(4,4,5,5-tetramethyl-1,3,2-dioxaborolan-2-yl)benzoate (1.4 g, 5.20 mmol) and 6-bromopyridin-3-amine (300 mg, 1.73 mmol) and Pd(dppf)Cl₂ (127 mg, 0.173 mmol) and tribasic potassium phosphate (1.10 g, 5.20 mmol) in THF (10.0 mL) and water (1.0 mL) was heated to 60° C. for 1 hour. Upon completion, the mixture was concentrated under reduced pressure. The crude residue was directly purified by silica gel column chromatography, eluted with petroleum ether/ethyl acetate (1:1) to afford methyl 4-(5-aminopyridin-2-yl)benzoate (220 mg, 55%) as a yellow solid. LCMS (ESI) [M+H]⁺: 229.

### Step 2: methyl 4-(5-(1-((4-isopropylphenyl)carbamoyl)-3,3-dimethylazetidine-2-carboxamido)pyridin-2-yl)benzoate

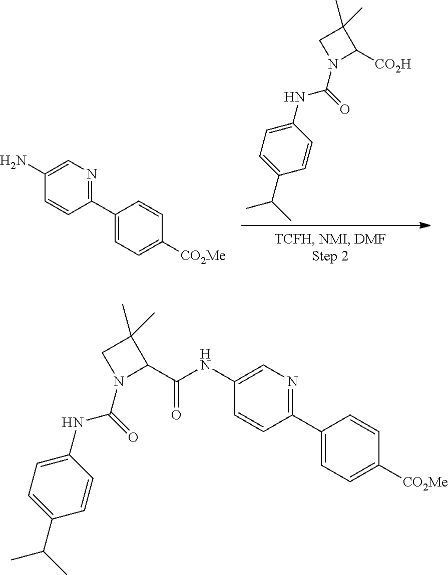 [SMILES: COC(=O)c1ccc(-c2ccc(N)cn2)cc1 ; CC(C)c1ccc(NC(=O)N2CC(C)(C)C2C(=O)O)cc1 ; COC(=O)c1ccc(-c2ccc(NC(=O)C3N(C(=O)Nc4ccc(C(C)C)cc4)CC3(C)C)cn2)cc1]

A solution of methyl 4-(5-aminopyridin-2-yl)benzoate (47.2 mg, 0.206 mmol), 1-methylimidazole (70.7 mg, 0.860 mmol), TCFH (96.6 mg, 0.344 mmol) and 1-((4-isopropylphenyl)carbamoyl)-3,3-dimethylazetidine-2-carboxylic acid (50.0 mg, 0.172 mmol) in MeCN (2.0 mL) was stirred at 25° C. for 1 hour. Upon completion, the mixture was concentrated under reduced pressure. The crude residue was directly purified by silica gel column chromatography, eluted with petroleum ether/ethyl acetate (1:1), to afford methyl 4-(5-(1-((4-isopropylphenyl)carbamoyl)-3,3-dimethylazetidine-2-carboxamido)pyridin-2-yl)benzoate (50 mg, 58%) as a yellow solid. LCMS (ESI) [M+H]⁺: 501.2.

### Step 3: 4-(5-(1-((4-isopropylphenyl)carbamoyl)-3,3-dimethylazetidine-2-carboxamido)pyridin-2-yl)benzoic acid

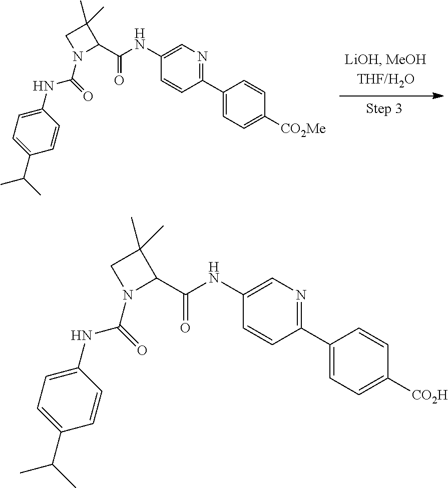 [SMILES: COC(=O)c1ccc(-c2ccc(NC(=O)C3N(C(=O)Nc4ccc(C(C)C)cc4)CC3(C)C)cn2)cc1 ; CC(C)c1ccc(NC(=O)N2CC(C)(C)C2C(=O)Nc2ccc(-c3ccc(C(=O)O)cc3)nc2)cc1]

A solution of methyl 4-(5-(1-((4-isopropylphenyl)carbamoyl)-3,3-dimethylazetidine-2-carboxamido)pyridin-2-yl)benzoate (50 mg, 0.100 mmol) and lithium hydroxide monohydrate (21.0 mg, 0.500 mmol) in methanol (1.0 mL), THF (1.0 mL) and water (1.0 mL) was stirred at 25° C. for 1 hour. Upon completion, the reaction mixture was concentrated under reduced pressure. The crude residue was directly purified by reversed phase chromatography (conditions: column: C18 silica gel; mobile phase A: water (0.1% formic acid); mobile phase B: MeCN; gradient (B %): 0% to 100% over 20 min; detector: UV 254 nm) to afford 4-(5-(1-((4-isopropylphenyl)carbamoyl)-3,3-dimethylazetidine-2-carboxamido)pyridin-2-yl)benzoic acid (40 mg, 82%) as a white solid. LCMS (ESI) [M+H]⁺: 487.2.

### Step 4: (S)-4-(5-(1-((4-isopropylphenyl)carbamoyl)-3,3-dimethylazetidine-2-carboxamido)pyridin-2-yl)benzoic acid and (R)-4-(5-(1-((4-isopropylphenyl)carbamoyl)-3,3-dimethylazetidine-2-carboxamido)pyridin-2-yl)benzoic acid

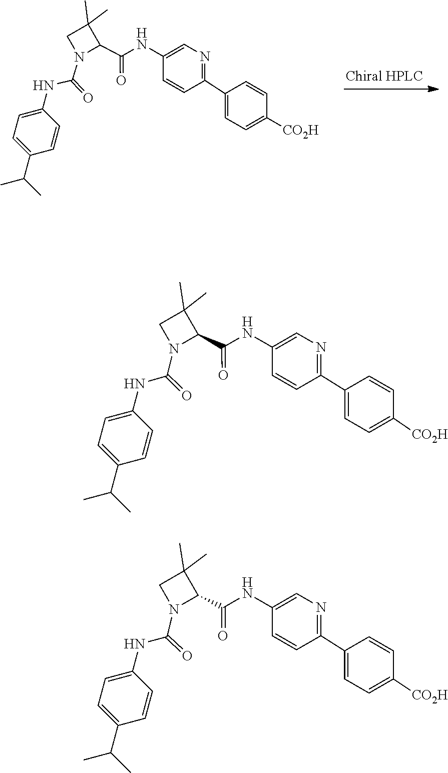 [SMILES: CC(C)c1ccc(NC(=O)N2CC(C)(C)C2C(=O)Nc2ccc(-c3ccc(C(=O)O)cc3)nc2)cc1 ; CC(C)c1ccc(NC(=O)N2CC(C)(C)[C@H]2C(=O)Nc2ccc(-c3ccc(C(=O)O)cc3)nc2)cc1 ; CC(C)c1ccc(NC(=O)N2CC(C)(C)[C@@H]2C(=O)Nc2ccc(-c3ccc(C(=O)O)cc3)nc2)cc1]

4-(5-(1-((4-isopropylphenyl)carbamoyl)-3,3-dimethylazetidine-2-carboxamido)pyridin-2-yl)benzoic acid (40 mg) was resolved by preparative chiral HPLC (conditions: column: CHIRAL ART Cellulose-SZ, 3*25 cm, 5 μm; mobile phase A: hexane (0.1% formic acid), mobile phase B: ethanol; flow rate: 40 mL/min; gradient (B %): isocratic, 50% B; detector: 220/254 nm; RT1 (min): 5.5; RT2 (min): 8.5) to afford (Compound 224a) (S)-4-(5-(1-((4-isopropylphenyl)carbamoyl)-3,3-dimethylazetidine-2-carboxamido)pyridin-2-yl)benzoic acid (1^{st} peak, 19.1 mg, 48%; Method BE, Peak 1, 0.63 min) as a white solid, and (Compound 224b) (R)-4-(5-(1-((4-isopropylphenyl)carbamoyl)-3,3-dimethylazetidine-2-carboxamido)pyridin-2-yl)benzoic acid (2^{nd} peak, 15.8 mg, 39%; Method BE, Peak 2, 0.95 min) as a white solid.

| Cmpd # | Characterization Data |
|---|---|
| 224a | LCMS (ESI) [M + H]⁺: 487.3 |
| | ¹H NMR (400 MHz, methanol-d₄) δ 8.90 (d, J = 2.5 Hz, 1H), 8.27 (dd, J = 8.7, 2.6 Hz, 1H), 8.11 |
| | (d, J = 8.5 Hz, 2H), 8.06 (d, J = 8.2 Hz, 2H), 7.93 (d, J = 8.6 Hz, 1H), 7.36 (d, J = 8.2 Hz, 2H), |
| | 7.14 (d, J = 8.6 Hz, 2H), 4.51 (d, J = 1.6 Hz, 1H), 3.86 (d, J = 7.3 Hz, 1H), 3.80 (d, J = 7.4 Hz, |
| | 1H), 2.85 (p, J = 6.9 Hz, 1H), 1.48 (s, 3H), 1.27 (s, 3H), 1.22 (s, 3H), 1.21 (s, 3H). |
| 224b | LCMS (ESI) [M + H]⁺: 487.3 |
| | ¹H NMR (400 MHz, methanol-d₄) δ 8.90 (d, J = 2.5 Hz, 1H), 8.30-8.22 (m, 1H), 8.14-8.08 (m, |
| | 2H), 8.08-8.02 (m, 2H), 7.98-7.86 (m, 1H), 7.36 (d, J = 8.5 Hz, 2H), 7.18-7.10 (m, 2H), 4.51 |
| | (d, J = 2.0 Hz, 1H), 3.86 (d, J = 7.4 Hz, 1H), 3.80 (d, J = 7.4 Hz, 1H), 2.90-2.78 (m, 1H), 1.47 |
| | (s, 3H), 1.27 (s, 3H), 1.22 (s, 3H), 1.20 (s, 3H). |

### Example 225: (S)-3-fluoro-4′-(1-((4-isopropylphenyl)carbamoyl)-3,3-dimethylazetidine-2-carboxamido)-[1,1′-biphenyl]-4-carboxylic acid and (R)-3-fluoro-4′-(1-((4-isopropylphenyl)carbamoyl)-3,3-dimethylazetidine-2-carboxamido)-[1,1′-biphenyl]-4-carboxylic acid

### Step 1: Methyl 4′-amino-3-fluoro-[1,1′-biphenyl]-4-carboxylate

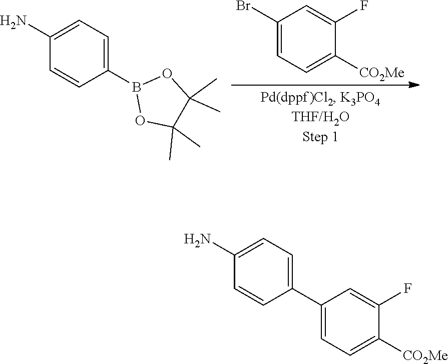 [SMILES: CC1(C)OB(c2ccc(N)cc2)OC1(C)C ; COC(=O)c1ccc(Br)cc1F ; COC(=O)c1ccc(-c2ccc(N)cc2)cc1F]

A solution of 4-(4,4,5,5-tetramethyl-1,3,2-dioxaborolan-2-yl)aniline (300 mg, 1.36 mmol), methyl 4-bromo-2-fluorobenzoate (382 mg, 1.64 mmol), tribasic potassium phosphate (872 mg, 4.10 mmol), and Pd(dppf)Cl₂ (111 mg, 0.137 mmol) in THF (5.0 mL) and water (1.0 mL) was heated to at 60° C. for 30 minutes. Upon completion, the reaction mixture was concentrated under reduced pressure. The crude residue was directly purified by silica gel column chromatography, eluted with dichloromethane/methanol (10:1) to afford methyl 4′-amino-3-fluoro-[1,1′-biphenyl]-4-carboxylate (280 mg, 83%) as a brown solid. LCMS (ESI) [M+H]⁺: 246.1.

### Step 2: methyl 3-fluoro-4′-(1-((4-isopropylphenyl)carbamoyl)-3,3-dimethylazetidine-2-carboxamido)-[1,1′-biphenyl]-4-carboxylate

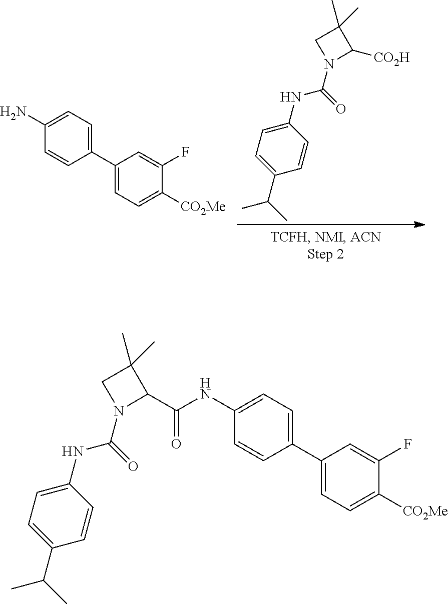 [SMILES: COC(=O)c1ccc(-c2ccc(N)cc2)cc1F ; CC(C)c1ccc(NC(=O)N2CC(C)(C)C2C(=O)O)cc1 ; COC(=O)c1ccc(-c2ccc(NC(=O)C3N(C(=O)Nc4ccc(C(C)C)cc4)CC3(C)C)cc2)cc1F]

A solution of methyl 4′-amino-3-fluoro-[1,1′-biphenyl]-4-carboxylate (50 mg, 0.204 mmol), 1-methylimidazole (83.7 mg, 1.02 mmol), TCFH (114 mg, 0.408 mmol), and 1-((4-isopropylphenyl)carbamoyl)-3,3-dimethylazetidine-2-carboxylic acid (71.0 mg, 0.245 mmol) in MeCN (2.0 mL) was stirred at 25° C. for 1 hour. Upon completion, the mixture was concentrated under reduced pressure. The crude residue was directly purified by silica gel column chromatography, eluted with petroleum ether/ethyl acetate (1:1), to afford methyl 3-fluoro-4′-(1-((4-isopropylphenyl)carbamoyl)-3,3-dimethylazetidine-2-carboxamido)-[1,1′-biphenyl]-4-carboxylate (65 mg, 57%) as a yellow solid. LCMS (ESI) [M+H]⁺: 518.2.

### Step 3: 3-fluoro-4′-(1-((4-isopropylphenyl)carbamoyl)-3,3-dimethylazetidine-2-carboxamido)-[1,1′-biphenyl]-4-carboxylic acid

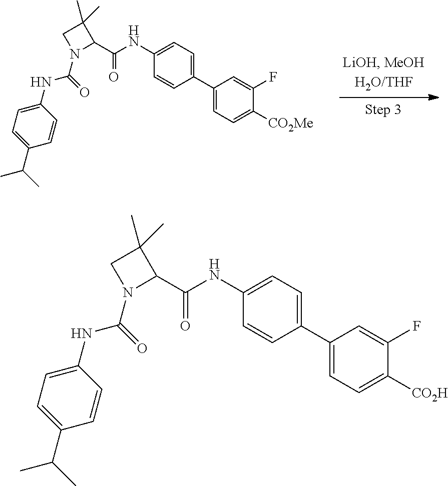 [SMILES: COC(=O)c1ccc(-c2ccc(NC(=O)C3N(C(=O)Nc4ccc(C(C)C)cc4)CC3(C)C)cc2)cc1F ; CC(C)c1ccc(NC(=O)N2CC(C)(C)C2C(=O)Nc2ccc(-c3ccc(C(=O)O)c(F)c3)cc2)cc1]

To a solution of methyl 3-fluoro-4′-(1-((4-isopropylphenyl)carbamoyl)-3,3-dimethylazetidine-2-carboxamido)-[1,1′-biphenyl]-4-carboxylate (65 mg, 0.126 mmol) in methanol (1.0 mL), THF (1.0 mL) and water (1.0 mL) was added lithium hydroxide monohydrate (26.5 mg, 0.630 mmol) at room temperature. After 2 hours, the reaction mixture was concentrated under reduced pressure. The crude residue was directly purified by reversed phase chromatography (conditions: column: C18 silica gel; mobile phase A: water (0.1% formic acid); mobile phase B: MeCN; gradient (B %): 0% to 100% over 20 min; detector: UV 254 nm) to afford 3-fluoro-4′-(1-((4-isopropylphenyl)carbamoyl)-3,3-dimethylazetidine-2-carboxamido)-[1,1′-biphenyl]-4-carboxylic acid (28 mg, 44%) as a white solid. LCMS (ESI) [M+H]⁺: 504.2.

### Step 4: (S)-3-fluoro-4′-(1-((4-isopropylphenyl)carbamoyl)-3,3-dimethylazetidine-2-carboxamido)-[1,1′-biphenyl]-4-carboxylic acid and (R)-3-fluoro-4′-(1-((4-isopropylphenyl)carbamoyl)-3,3-dimethylazetidine-2-carboxamido)-[1,1′-biphenyl]-4-carboxylic acid

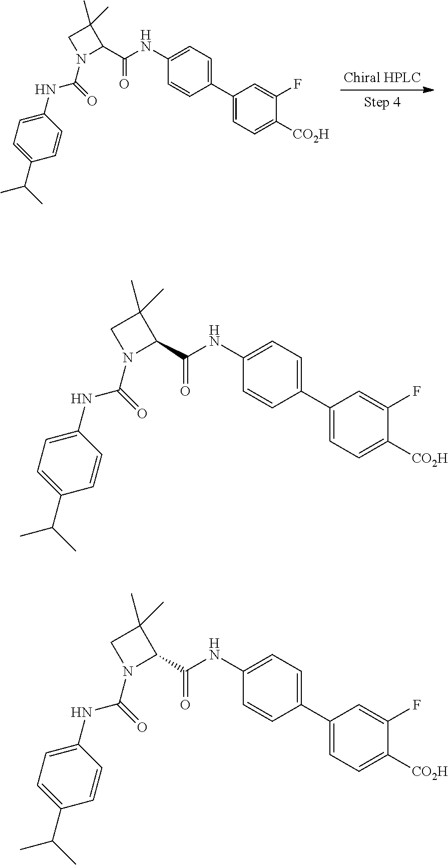 [SMILES: CC(C)c1ccc(NC(=O)N2CC(C)(C)C2C(=O)Nc2ccc(-c3ccc(C(=O)O)c(F)c3)cc2)cc1 ; CC(C)c1ccc(NC(=O)N2CC(C)(C)[C@H]2C(=O)Nc2ccc(-c3ccc(C(=O)O)c(F)c3)cc2)cc1 ; CC(C)c1ccc(NC(=O)N2CC(C)(C)[C@@H]2C(=O)Nc2ccc(-c3ccc(C(=O)O)c(F)c3)cc2)cc1]

3-fluoro-4′-(1-((4-isopropylphenyl)carbamoyl)-3,3-dimethylazetidine-2-carboxamido)-[1,1′-biphenyl]-4-carboxylic acid (28 mg) was resolved by preparative chiral HPLC (conditions: column: CHIRAL ART Cellulose-SZ, 3*25 cm, 5 μm; mobile phase A: MTBE (0.1% formic acid), mobile phase B: methanol; flow rate: 40 mL/min; gradient (B %): isocratic, 25% B; detector: 220/254 nm; RT1 (min): 6.3; RT2 (min): 14.3) to afford (Compound 225a) (S)-3-fluoro-4′-(1-((4-isopropylphenyl)carbamoyl)-3,3-dimethylazetidine-2-carboxamido)-[1,1′-biphenyl]-4-carboxylic acid (1^{st} peak, 9.8 mg, 35%; Method BJ, Peak 1, 0.66 min) as a white solid and (Compound 225b) (R)-3-fluoro-4′-(1-((4-isopropylphenyl)carbamoyl)-3,3-dimethylazetidine-2-carboxamido)-[1,1′-biphenyl]-4-carboxylic acid (2^{nd} peak, 10.2 mg, 36%; Method BJ, Peak 2, 1.53 min) as a white solid.

| Cmpd # | Characterization Data |
|---|---|
| 225a | LCMS (ESI) [M + H]⁺: 504.3 |
| | ¹H NMR (400 MHz, methanol-d₄) δ 7.97 (td, J = 8.1, 3.4 Hz, 1H), 7.79-7.72 (m, 2H), |
| | 7.67 (ddd, J = 12.3, 7.4, 3.1 Hz, 2H), 7.56-7.41 (m, 2H), 7.36 (d, J = 8.5 Hz, 2H), 7.17- |
| | 7.08 (m, 2H), 4.49 (d, J = 1.5 Hz, 1H), 3.85 (d, J = 7.2 Hz, 1H), 3.77 (dd, J = 7.4, 2.1 |
| | Hz, 1H), 2.90-2.77 (m, 1H), 1.49-1.42 (m, 3H), 1.26 (d, J = 2.6 Hz, 3H), 1.21 (ddd, J = |
| | 6.9, 3.2, 1.2 Hz, 6H). |
| 225b | LCMS (ESI) [M + H]⁺: 504.3 |
| | ¹H NMR (400 MHz, methanol-d₄) δ 7.96 (t, J = 7.9 Hz, 1H), 7.75 (dt, J = 8.6, 1.8 Hz, |
| | 2H), 7.66 (tt, J = 8.9, 6.9, 2.9 Hz, 2H), 7.57-7.40 (m, 2H), 7.36 (d, J = 8.5 Hz, 2H), |
| | 7.17-7.08 (m, 2H), 4.49 (s, 1H), 3.85 (d, J = 7.3 Hz, 1H), 3.80-3.74 (m, 1H), 2.83 (h, |
| | J = 5.3, 4.8 Hz, 1H), 1.46 (dd, J = 3.8, 2.0 Hz, 3H), 1.26 (d, J = 1.7 Hz, 3H), 1.24-1.17 |
| | (m, 6H). |

### Example 226: (S)-4′-(1-((4-isopropylphenyl)carbamoyl)-3,3-dimethylazetidine-2-carboxamido)-2-methoxy-[1,1′-biphenyl]-4-carboxylic acid and (R)-4′-(1-((4-isopropylphenyl)carbamoyl)-3,3-dimethylazetidine-2-carboxamido)-2-methoxy-[1,1′-biphenyl]-4-carboxylic acid

### Step 1: Methyl 4′-amino-2-methoxy-[1,1′-biphenyl]-4-carboxylate

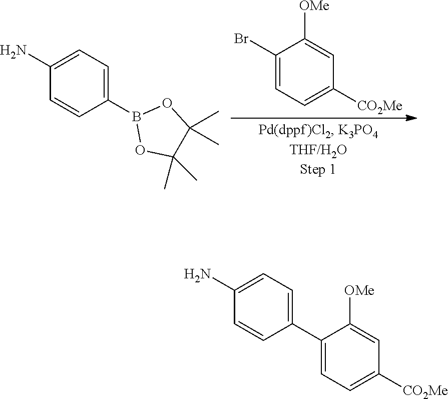 [SMILES: CC1(C)OB(c2ccc(N)cc2)OC1(C)C ; COC(=O)c1ccc(Br)c(OC)c1 ; COC(=O)c1ccc(-c2ccc(N)cc2)c(OC)c1]

A solution of 4-(4,4,5,5-tetramethyl-1,3,2-dioxaborolan-2-yl)aniline (700 mg, 3.19 mmol), methyl 4-bromo-3-methoxybenzoate (861 mg, 3.51 mmol), Pd(dppf)Cl₂ (234 mg, 0.32 mmol), and tribasic potassium phosphate (2.00 g, 9.58 mmol) in THF (10.0 mL) and water (1.0 mL) was heated to 80° C. for 2 hours. Upon completion, the reaction mixture was concentrated under reduced pressure. The crude residue was directly purified by silica gel column chromatography, eluted with petroleum ether/ethyl acetate (1:1), to afford methyl 4′-amino-2-methoxy-[1,1′-biphenyl]-4-carboxylate (500 mg, 60%) as a pale-yellow solid. LCMS (ESI) [M+H]⁺: 258.1.

### Step 2: methyl 4′-(1-((4-isopropylphenyl)carbamoyl)-3,3-dimethylazetidine-2-carboxamido)-2-methoxy-[1,1′-biphenyl]-4-carboxylate

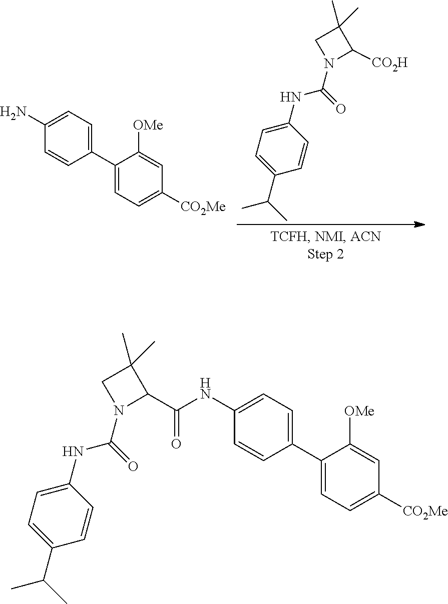 [SMILES: COC(=O)c1ccc(-c2ccc(N)cc2)c(OC)c1 ; CC(C)c1ccc(NC(=O)N2CC(C)(C)C2C(=O)O)cc1 ; COC(=O)c1ccc(-c2ccc(NC(=O)C3N(C(=O)Nc4ccc(C(C)C)cc4)CC3(C)C)cc2)c(OC)c1]

A solution of methyl 4′-amino-2-methoxy-[1,1′-biphenyl]-4-carboxylate (48.7 mg, 0.18 mmol), 1-methylimidazole (42.4 mg, 0.51 mmol), TCFH (72.4 mg, 0.25 mmol) and 1-((4-isopropylphenyl)carbamoyl)-3,3-dimethylazetidine-2-carboxylic acid (50.0 mg, 0.17 mmol) in MeCN (2.0 mL) was stirred at 25° C. for 1 hour. Upon completion, the mixture was concentrated under reduced pressure. The crude residue was directly purified by silica gel column chromatography, eluted with petroleum ether/ethyl acetate (1:1), to afford methyl 4′-(1-((4-isopropylphenyl)carbamoyl)-3,3-dimethylazetidine-2-carboxamido)-2-methoxy-[1,1′-biphenyl]-4-carboxylate (60 mg, 65%) as a yellow solid. LCMS (ESI) [M+H]⁺: 530.2.

### Step 3: 4′-(1-((4-isopropylphenyl)carbamoyl)-3,3-dimethylazetidine-2-carboxamido)-2-methoxy-[1,1′-biphenyl]-4-carboxylic acid

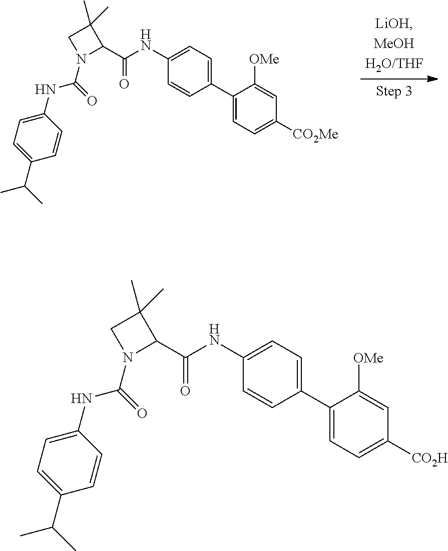 [SMILES: COC(=O)c1ccc(-c2ccc(NC(=O)C3N(C(=O)Nc4ccc(C(C)C)cc4)CC3(C)C)cc2)c(OC)c1 ; COc1cc(C(=O)O)ccc1-c1ccc(NC(=O)C2N(C(=O)Nc3ccc(C(C)C)cc3)CC2(C)C)cc1]

To methyl 4′-(1-((4-isopropylphenyl)carbamoyl)-3,3-dimethylazetidine-2-carboxamido)-2-methoxy-[1,1′-biphenyl]-4-carboxylate (50.0 mg, 0.09 mmol) in a solution of methanol (1.0 mL), THF (1.0 mL) and water (1.0 mL) was added lithium hydroxide monohydrate (78.9 mg, 1.88 mmol) at room temperature. After 2 hours, the reaction mixture was concentrated under reduced pressure. The crude residue was directly purified by reversed phase chromatography (conditions: column: C18 silica gel; mobile phase A: water (0.1% formic acid); mobile phase B: MeCN; gradient (B %): 0% to 100% over 20 min; detector: UV 254 nm) to afford 4′-(1-((4-isopropylphenyl)carbamoyl)-3,3-dimethylazetidine-2-carboxamido)-2-methoxy-[1,1′-biphenyl]-4-carboxylic acid (37 mg, 76%) as a white solid. LCMS (ESI) [M+H]⁺: 516.2.

### Step 4: (S)-4′-(1-((4-isopropylphenyl)carbamoyl)-3,3-dimethylazetidine-2-carboxamido)-2-methoxy-[1,1′-biphenyl]-4-carboxylic acid and (R)-4′-(1-((4-isopropylphenyl)carbamoyl)-3,3-dimethylazetidine-2-carboxamido)-2-methoxy-[1,1′-biphenyl]-4-carboxylic ac

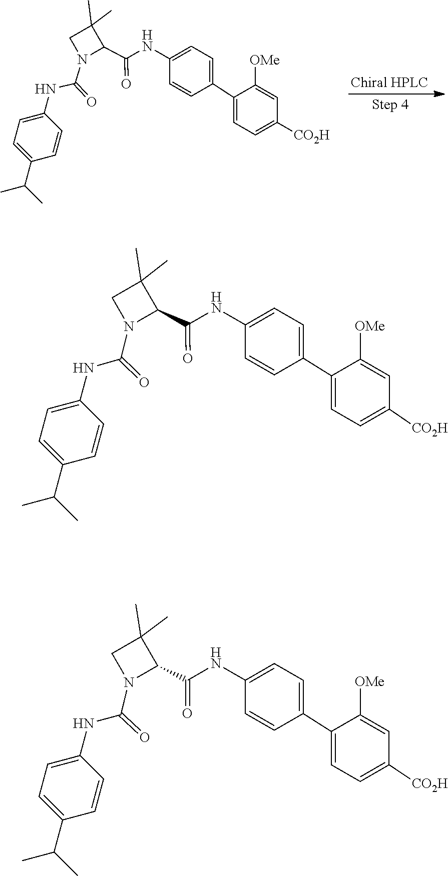 [SMILES: COc1cc(C(=O)O)ccc1-c1ccc(NC(=O)C2N(C(=O)Nc3ccc(C(C)C)cc3)CC2(C)C)cc1 ; COc1cc(C(=O)O)ccc1-c1ccc(NC(=O)[C@H]2N(C(=O)Nc3ccc(C(C)C)cc3)CC2(C)C)cc1 ; COc1cc(C(=O)O)ccc1-c1ccc(NC(=O)[C@@H]2N(C(=O)Nc3ccc(C(C)C)cc3)CC2(C)C)cc1]

4′-(1-((4-isopropylphenyl)carbamoyl)-3,3-dimethylazetidine-2-carboxamido)-2-methoxy-[1,1′-biphenyl]-4-carboxylic acid (37 mg) was resolved by preparative chiral HPLC (conditions: Column: CHIRAL ART Cellulose-SZ, 3*25 cm, 5 μm; mobile phase A: hexane (0.1% formic acid), mobile phase B: ethanol; flow rate: 40 mL/min; gradient (B %): isocratic, 50% B; detector: 220/254 nm; RT1 (min): 7.3; RT2 (min): 22.5) to afford (Compound 226a) (S)-4′-(1-((4-isopropylphenyl)carbamoyl)-3,3-dimethylazetidine-2-carboxamido)-2-methoxy-[1,1′-biphenyl]-4-carboxylic acid (1^{st} peak, 11.1 mg, 30%; Method BE, Peak 1, 0.80 min) as a white solid, and (Compound 226b) (R)-4′-(1-((4-isopropylphenyl)carbamoyl)-3,3-dimethylazetidine-2-carboxamido)-2-methoxy-[1,1′-biphenyl]-4-carboxylic acid (2^{nd} peak, 11.1 mg, 30%; Method BE, Peak 2, 2.84 min) as a white solid.

| Cmpd # | Characterization Data |
|---|---|
| 226a | LCMS (ESI) [M + H]⁺: 516.3 |
| | ¹H NMR (400 MHz, methanol-d₄) δ 7.72-7.63 (m, 4H), 7.51 (d, J = 8.7 Hz, 2H), 7.38 (dd, J = |
| | 16.8, 8.5 Hz, 3H), 7.14 (d, J = 8.5 Hz, 2H), 4.48 (s, 1H), 3.86 (s, 4H), 3.78 (d, J = 7.4 Hz, 1H), |
| | 2.85 (p, J = 6.9 Hz, 1H), 1.47 (s, 3H), 1.27 (s, 3H), 1.23 (s, 3H), 1.21 (s, 3H). |
| 226b | LCMS (ESI) [M + H]⁺: 516.3 |
| | ¹H NMR (400 MHz, methanol-d₄) δ 7.72-7.62 (m, 4H), 7.54-7.48 (m, 2H), 7.41-7.32 (m, |
| | 3H), 7.18-7.11 (m, 2H), 4.48 (s, 1H), 3.85 (d, J = 7.4 Hz, 4H), 3.78 (d, J = 7.4 Hz, 1H), 2.85 (p, |
| | J = 6.9 Hz, 1H), 1.47 (s, 3H), 1.27 (s, 3H), 1.23 (s, 3H), 1.21 (s, 3H). |

### Example 227: (S)-4′-(1-((4-isopropylphenyl)carbamoyl)-3,3-dimethylazetidine-2-carboxamido)-[1,1′-biphenyl]-4-carboxylic acid and (R)-4′-(1-((4-isopropylphenyl)carbamoyl)-3,3-dimethylazetidine-2-carboxamido)-[1,1′-biphenyl]-4-carboxylic acid

### Step 1: methyl 4′-amino-[1,1′-biphenyl]-4-carboxylate

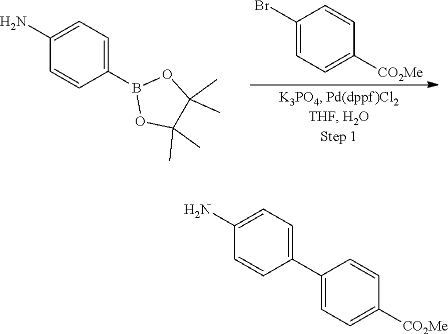 [SMILES: CC1(C)OB(c2ccc(N)cc2)OC1(C)C ; COC(=O)c1ccc(Br)cc1 ; COC(=O)c1ccc(-c2ccc(N)cc2)cc1]

A solution of 4-(4,4,5,5-tetramethyl-1,3,2-dioxaborolan-2-yl)aniline (100 mg, 0.456 mmol), methyl 4-bromobenzoate (98 mg, 0.456 mmol), tribasic potassium phosphate (291 mg, 1.37 mmol) and Pd(dppf)Cl₂ (33.0 mg, 0.045 mmol) in THF (1.0 mL) and water (0.10 mL) was heated to 80° C. for 3 hours. Upon completion, the mixture was concentrated under reduced pressure. The crude residue was directly purified by silica gel column chromatography, eluted with petroleum ether/ethyl acetate (3:1) to afford methyl 4′-amino-[1,1′-biphenyl]-4-carboxylate (90 mg, 85%) as a white solid. LCMS (ESI) [M+H]⁺: 228.0.

### Step 2: methyl 4′-(1-((4-isopropylphenyl)carbamoyl)-3,3-dimethylazetidine-2-carboxamido)-[1,1′-biphenyl]-4-carboxylate

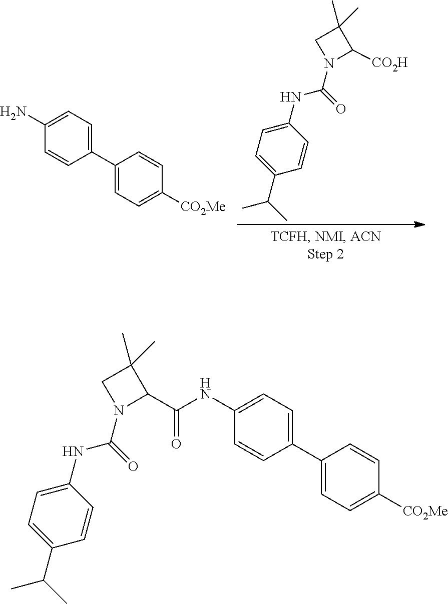 [SMILES: COC(=O)c1ccc(-c2ccc(N)cc2)cc1 ; CC(C)c1ccc(NC(=O)N2CC(C)(C)C2C(=O)O)cc1 ; COC(=O)c1ccc(-c2ccc(NC(=O)C3N(C(=O)Nc4ccc(C(C)C)cc4)CC3(C)C)cc2)cc1]

A solution of methyl 4′-amino-[1,1′-biphenyl]-4-carboxylate (90 mg, 0.396 mmol), 1-methylimidazole (97.5 mg, 1.19 mmol), TCFH (144 mg, 0.515 mmol), and 1-((4-isopropylphenyl)carbamoyl)-3,3-dimethylazetidine-2-carboxylic acid (115 mg, 0.396 mmol) in MeCN (2.0 mL) was stirred at 25° C. for 1 hour. Upon completion, the mixture was concentrated under reduced pressure. The crude residue was directly purified by silica gel column chromatography, eluted with petroleum ether/ethyl acetate (1:1), to afford methyl 4′-(1-((4-isopropylphenyl)carbamoyl)-3,3-dimethylazetidine-2-carboxamido)-[1,1′-biphenyl]-4-carboxylate (90 mg, 45%) as a yellow solid. LCMS (ESI) [M+H]⁺: 500.2.

### Step 3: 4′-(1-((4-isopropylphenyl)carbamoyl)-3,3-dimethylazetidine-2-carboxamido)-[1,1′-biphenyl]-4-carboxylic acid

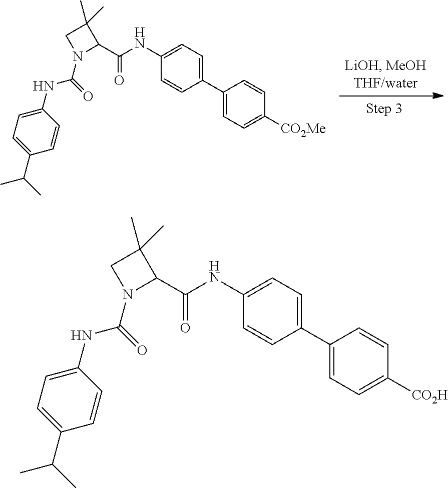 [SMILES: COC(=O)c1ccc(-c2ccc(NC(=O)C3N(C(=O)Nc4ccc(C(C)C)cc4)CC3(C)C)cc2)cc1 ; CC(C)c1ccc(NC(=O)N2CC(C)(C)C2C(=O)Nc2ccc(-c3ccc(C(=O)O)cc3)cc2)cc1]

To methyl 4′-(1-((4-isopropylphenyl)carbamoyl)-3,3-dimethylazetidine-2-carboxamido)-[1,1′-biphenyl]-4-carboxylate (90.0 mg, 0.180 mmol) in a solution of methanol (1.0 mL), THF (1.0 mL) and water (1.0 mL) was added lithium hydroxide monohydrate (37.8 mg, 0.9 mmol) at room temperature. After 4 hours, the reaction mixture was concentrated under reduced pressure. The crude residue was directly purified by reversed phase chromatography (conditions: column: C18 silica gel; mobile phase A: water (0.1% formic acid); mobile phase B: MeCN; gradient (B %): 0% to 100% over 20 min; detector: UV 254 nm) to afford 4′-(1-((4-isopropylphenyl)carbamoyl)-3,3-dimethylazetidine-2-carboxamido)-[1,1′-biphenyl]-4-carboxylic acid (60 mg, 68%) as a white solid. LCMS (ESI) [M+H]⁺: 486.2.

### Step 4: (S)-4′-(1-((4-isopropylphenyl)carbamoyl)-3,3-dimethylazetidine-2-carboxamido)-[1,1′-biphenyl]-4-carboxylic acid and (R)-4′-(1-((4-isopropylphenyl)carbamoyl)-3,3-dimethylazetidine-2-carboxamido)-[1,1′-biphenyl]-4-carboxylic acid

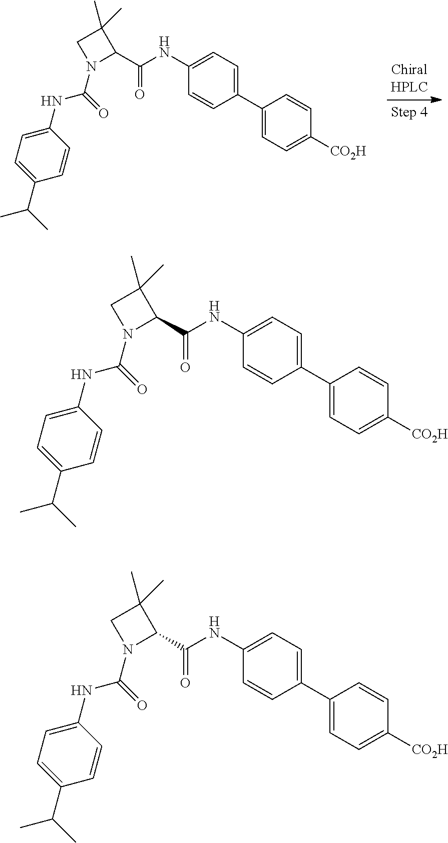 [SMILES: CC(C)c1ccc(NC(=O)N2CC(C)(C)C2C(=O)Nc2ccc(-c3ccc(C(=O)O)cc3)cc2)cc1 ; CC(C)c1ccc(NC(=O)N2CC(C)(C)[C@H]2C(=O)Nc2ccc(-c3ccc(C(=O)O)cc3)cc2)cc1 ; CC(C)c1ccc(NC(=O)N2CC(C)(C)[C@@H]2C(=O)Nc2ccc(-c3ccc(C(=O)O)cc3)cc2)cc1]

4′-(1-((4-isopropylphenyl)carbamoyl)-3,3-dimethylazetidine-2-carboxamido)-[1,1′-biphenyl]-4-carboxylic acid (50 mg) was resolved by preparative chiral HPLC (conditions: column: CHIRAL ART Cellulose-SZ, 3*25 cm, 5 μm; mobile phase A: MTBE (0.1% formic acid), mobile phase B: methanol; flow rate: 40 mL/min; gradient (B %): isocratic, 50% B; detector: 220/254 nm; RT1 (min): 5.4; RT2 (min): 8.3) to afford (Compound 227a) (S)-4′-(1-((4-isopropylphenyl)carbamoyl)-3,3-dimethylazetidine-2-carboxamido)-[1,1′-biphenyl]-4-carboxylic acid (1^{st} peak, 18.8 mg, 37%; Method BL, Peak 1, 0.52 min) as a white solid and (Compound 227b) (R)-4′-(1-((4-isopropylphenyl)carbamoyl)-3,3-dimethylazetidine-2-carboxamido)-[1,1′-biphenyl]-4-carboxylic acid (2^{nd} peak, 20.2 mg, 40%; Method BL, Peak 2, 0.93 min) as a white solid.

| Cmpd # | Characterization Data |
|---|---|
| 227a | LCMS (ESI) [M + H]⁺: 486.3 |
| | ¹H NMR (400 MHz, methanol-d₄) δ 8.08 (d, J = 8.5 Hz, 2H), 7.77-7.71 (m, 4H), 7.71-7.65 (m, |
| | 2H), 7.36 (d, J = 8.6 Hz, 2H), 7.14 (d, J = 8.5 Hz, 2H), 4.49 (s, 1H), 3.85 (d, J = 7.3 Hz, 1H), |
| | 3.78 (d, J = 7.3 Hz, 1H), 2.85 (p, J = 6.9 Hz, 1H), 1.47 (s, 3H), 1.27 (s, 3H), 1.23 (s, 3H), 1.21 (s, |
| | 3H). |
| 227b | LCMS (ESI) [M + H]⁺: 486.3 |
| | ¹H NMR (400 MHz, methanol-d₄) δ 8.07 (d, J = 8.1 Hz, 2H), 7.76-7.70 (m, 4H), 7.67 (d, J = |
| | 8.8 Hz, 2H), 7.36 (d, J = 8.6 Hz, 2H), 7.14 (d, J = 8.6 Hz, 2H), 4.49 (s, 1H), 3.85 (d, J = 7.4 Hz, |
| | 1H), 3.78 (d, J = 7.4 Hz, 1H), 2.85 (p, J = 6.9 Hz, 1H), 1.47 (s, 3H), 1.27 (s, 3H), 1.23 (s, 3H), |
| | 1.21 (s, 3H). |

### Example 228: (S)-4-(3-fluoro-5-(1-((4-isopropylphenyl)carbamoyl)-3,3-dimethylazetidine-2-carboxamido)pyridin-2-yl)benzoic acid and (R)-4-(3-fluoro-5-(1-((4-isopropylphenyl)carbamoyl)-3,3-dimethylazetidine-2-carboxamido)pyridin-2-yl)benzoic acid

### Step 1: Methyl 4-(5-amino-3-fluoropyridin-2-yl)benzoate

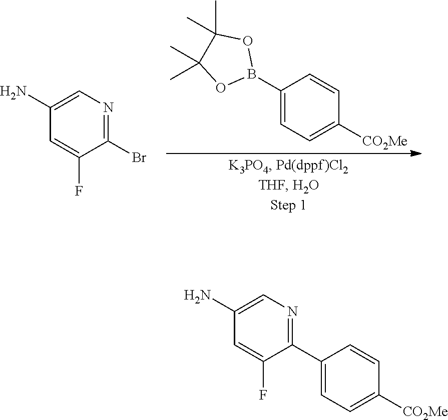 [SMILES: Nc1cnc(Br)c(F)c1 ; COC(=O)c1ccc(B2OC(C)(C)C(C)(C)O2)cc1 ; COC(=O)c1ccc(-c2ncc(N)cc2F)cc1]

A solution of methyl 4-(4,4,5,5-tetramethyl-1,3,2-dioxaborolan-2-yl)benzoate (823 mg, 3.14 mmol), 6-bromo-5-fluoropyridin-3-amine (300 mg, 1.57 mmol), Pd(dppf)Cl₂ (115 mg, 0.157 mmol), and tribasic potassium phosphate (1.00 g, 4.71 mmol) in THF (5.0 mL) and water (1.0 mL) was heated to 60° C. for 1 hour. Upon completion, the reaction mixture was concentrated under reduced pressure. The crude residue was directly purified by silica gel column chromatography, eluted with petroleum ether/ethyl acetate (1:1) to afford methyl 4-(5-amino-3-fluoropyridin-2-yl)benzoate (200 mg, 52%) as a yellow solid. LCMS (ESI) [M+H]⁺: 247.1.

### Step 2: methyl 4-(3-fluoro-5-(1-((4-isopropylphenyl)carbamoyl)-3,3-dimethylazetidine-2-carboxamido)pyridin-2-yl)benzoate

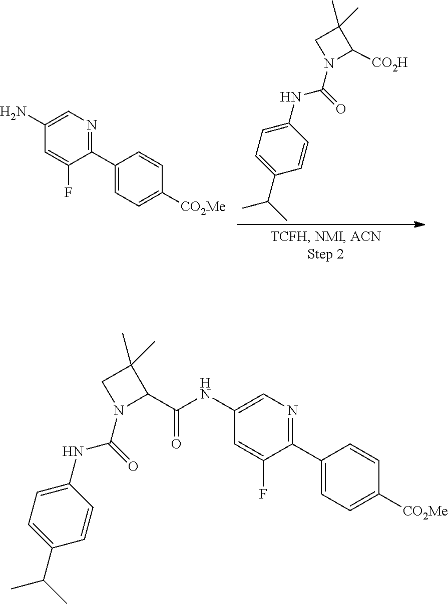 [SMILES: COC(=O)c1ccc(-c2ncc(N)cc2F)cc1 ; CC(C)c1ccc(NC(=O)N2CC(C)(C)C2C(=O)O)cc1 ; COC(=O)c1ccc(-c2ncc(NC(=O)C3N(C(=O)Nc4ccc(C(C)C)cc4)CC3(C)C)cc2F)cc1]

A solution of methyl 4-(5-amino-3-fluoropyridin-2-yl)benzoate (51.0 mg, 0.206 mmol), 1-methylimidazole (70.7 mg, 0.860 mmol), TCFH (96.6 mg, 0.344 mmol) and 1-((4-isopropylphenyl)carbamoyl)-3,3-dimethylazetidine-2-carboxylic acid (50.0 mg, 0.172 mmol) in MeCN (2.0 mL) was stirred at 25° C. for 1 hour. Upon completion, the mixture was concentrated under reduced pressure. The crude residue was directly purified by silica gel column chromatography, eluted with petroleum ether/ethyl acetate (1:1), to afford methyl 4-(3-fluoro-5-(1-((4-isopropylphenyl)carbamoyl)-3,3-dimethylazetidine-2-carboxamido)pyridin-2-yl)benzoate (60 mg, 67%) as a yellow solid. LCMS (ESI) [M+H]⁺: 518.2.

### Step 3: 4-(3-fluoro-5-(1-((4-isopropylphenyl)carbamoyl)-3,3-dimethylazetidine-2-carboxamido)pyridin-2-yl)benzoic acid

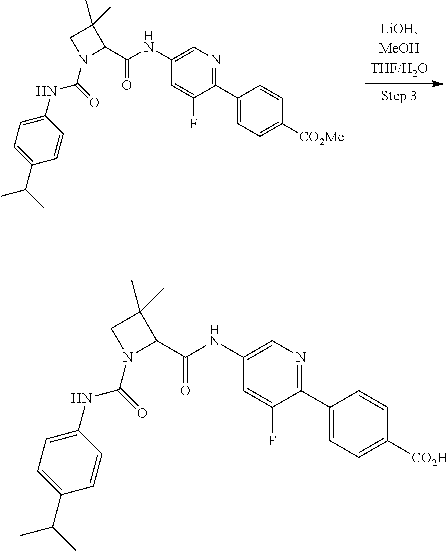 [SMILES: COC(=O)c1ccc(-c2ncc(NC(=O)C3N(C(=O)Nc4ccc(C(C)C)cc4)CC3(C)C)cc2F)cc1 ; CC(C)c1ccc(NC(=O)N2CC(C)(C)C2C(=O)Nc2cnc(-c3ccc(C(=O)O)cc3)c(F)c2)cc1]

To methyl 4-(3-fluoro-5-(1-((4-isopropylphenyl)carbamoyl)-3,3-dimethylazetidine-2-carboxamido)pyridin-2-yl)benzoate (60.0 mg, 0.116 mmol) in a solution of methanol (1.0 mL), THF (1.0 mL), and water (1.0 mL) was added lithium hydroxide monohydrate (24.4 mg, 0.580 mmol) at room temperature. After 4 hours, the reaction mixture was concentrated under reduced pressure. The crude residue was directly purified by reversed phase chromatography (conditions: column: C18 silica gel; mobile phase A: water (0.1% formic acid); mobile phase B: MeCN; gradient (B %): 0% to 100% over 20 min; detector: UV 254 nm) to afford 4-(3-fluoro-5-(1-((4-isopropylphenyl)carbamoyl)-3,3-dimethylazetidine-2-carboxamido)pyridin-2-yl)benzoic acid (40 mg, 68%) as a white solid. LCMS (ESI) [M+H]⁺: 505.2.

### Step 4: (S)-4-(3-fluoro-5-(1-((4-isopropylphenyl)carbamoyl)-3,3-dimethylazetidine-2-carboxamido)pyridin-2-yl)benzoic acid and (R)-4-(3-fluoro-5-(1-((4-isopropylphenyl)carbamoyl)-3,3-dimethylazetidine-2-carboxamido)pyridin-2-yl)benzoic acid

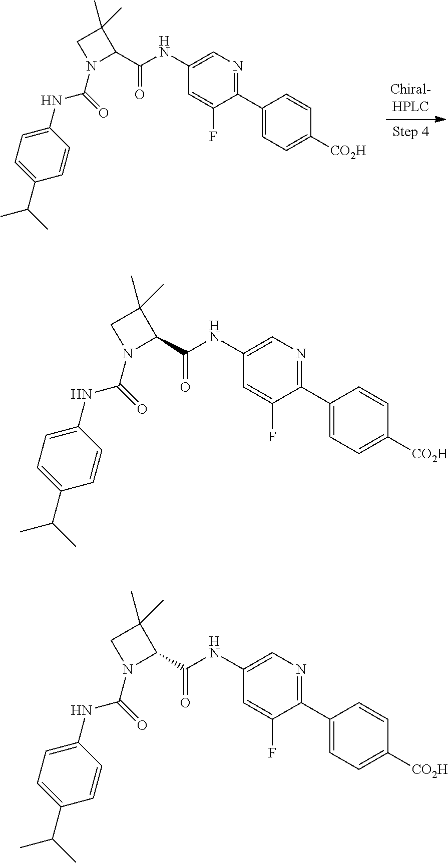 [SMILES: CC(C)c1ccc(NC(=O)N2CC(C)(C)C2C(=O)Nc2cnc(-c3ccc(C(=O)O)cc3)c(F)c2)cc1 ; CC(C)c1ccc(NC(=O)N2CC(C)(C)[C@H]2C(=O)Nc2cnc(-c3ccc(C(=O)O)cc3)c(F)c2)cc1 ; CC(C)c1ccc(NC(=O)N2CC(C)(C)[C@@H]2C(=O)Nc2cnc(-c3ccc(C(=O)O)cc3)c(F)c2)cc1]

4-(3-fluoro-5-(1-((4-isopropylphenyl)carbamoyl)-3,3-dimethylazetidine-2-carboxamido)pyridin-2-yl)benzoic acid (40 mg) was resolved by preparative chiral HPLC (conditions: column: CHIRAL ART Cellulose-SZ, 3*25 cm, 5 μm; mobile phase A: hexane (0.1% formic acid), mobile phase B: ethanol; flow rate: 40 mL/min; gradient (B %): isocratic, 25% B; detector: 220/254 nm; RT1 (min): 6.4; RT2 (min): 9.0) to afford (Compound 228a) (S)-4-(3-fluoro-5-(1-((4-isopropylphenyl)carbamoyl)-3,3-dimethylazetidine-2-carboxamido)pyridin-2-yl)benzoic acid (1^{st} peak, 14.6 mg, 36%; Method BM, Peak 1, 0.68 min) as a white solid and (Compound 228b) (R)-4-(3-fluoro-5-(1-((4-isopropylphenyl)carbamoyl)-3,3-dimethylazetidine-2-carboxamido)pyridin-2-yl)benzoic acid (2^{nd} peak, 14.5 mg, 36%; Method BM, Peak 2, 1.00 min) as a white solid.

| Cmpd # | Characterization Data |
|---|---|
| 228a | LCMS (ESI) [M + H]⁺: 505.3 |
| | ¹H NMR (400 MHz, methanol-d₄) δ 8.71 (dd, J = 2.1, 1.2 Hz, 1H), 8.29-8.22 (m, 1H), 8.15- |
| | 8.09 (m, 2H), 8.02-7.96 (m, 2H), 7.39-7.32 (m, 2H), 7.14 (dd, J = 8.4, 1.9 Hz, 2H), 4.51 (d, J = |
| | 1.3 Hz, 1H), 3.88-3.74 (m, 2H), 2.89-2.81 (m, 1H), 1.47 (d, J = 1.7 Hz, 3H), 1.26 (s, 3H), |
| | 1.22 (d, J = 1.4 Hz, 3H), 1.21 (d, J = 1.5 Hz, 3H). |
| 228b | LCMS (ESI) [M + H]⁺: 505.3 |
| | ¹H NMR (400 MHz, methanol-d₄) δ 8.71 (dd, J = 2.2, 1.2 Hz, 1H), 8.25 (dt, J = 13.2, 2.3 Hz, |
| | 1H), 8.12 (d, J = 8.1 Hz, 2H), 7.99 (dt, J = 8.4, 1.8 Hz, 2H), 7.36 (d, J = 8.5 Hz, 2H), 7.17-7.11 |
| | (m, 2H), 4.51 (s, 1H), 3.89-3.76 (m, 2H), 1.47 (s, 3H), 1.26 (s, 3H), 1.22 (s, 3H), 1.21 (s, 3H). |

### Example 229: (S)-4′-(1-((4-isopropylphenyl)carbamoyl)-3,3-dimethylazetidine-2-carboxamido)-3-methyl-[1,1′-biphenyl]-4-carboxylic acid and (R)-4′-(1-((4-isopropylphenyl)carbamoyl)-3,3-dimethylazetidine-2-carboxamido)-3-methyl-[1,1′-biphenyl]-4-carboxylic acid

### Step 1: methyl 4′-amino-3-methyl-[1,1′-biphenyl]-4-carboxylate

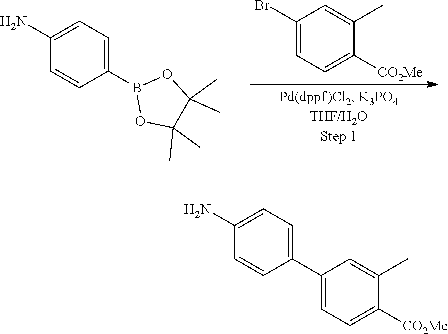 [SMILES: CC1(C)OB(c2ccc(N)cc2)OC1(C)C ; COC(=O)c1ccc(Br)cc1C ; COC(=O)c1ccc(-c2ccc(N)cc2)cc1C]

A mixture of 4-(4,4,5,5-tetramethyl-1,3,2-dioxaborolan-2-yl)aniline (300 mg, 1.36 mmol), methyl 4-bromo-2-methylbenzoate (376 mg, 1.643 mmol), Pd(dppf)Cl₂ (111 mg, 0.137 mmol), and tribasic potassium phosphate (871 mg, 4.10 mmol) in THF (5.0 mL) and water (1.0 mL) was stirred at 60° C. for 4 hours. Upon completion, the reaction mixture was concentrated under reduced pressure. The crude directly residue was purified by silica gel column chromatography, eluted with petroleum ether/ethyl acetate (1:1) to afford methyl 4′-amino-3-methyl-[1,1′-biphenyl]-4-carboxylate (274 mg, 83%) as a yellow solid. LCMS (ESI) [M+H]⁺: 228.1.

### Step 2: methyl 4′-(1-((4-isopropylphenyl)carbamoyl)-3,3-dimethylazetidine-2-carboxamido)-3-methyl-[1,1′-biphenyl]-4-carboxylate

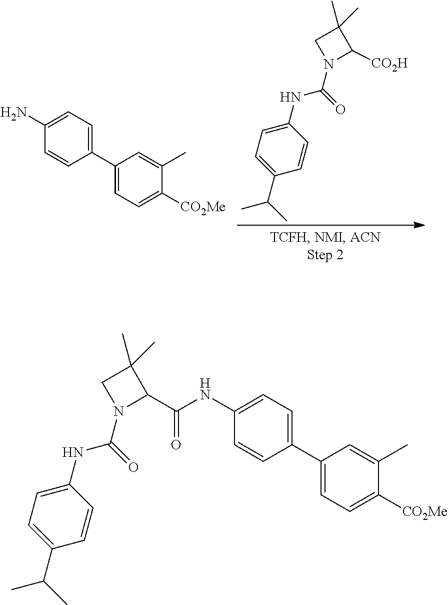 [SMILES: COC(=O)c1ccc(-c2ccc(N)cc2)cc1C ; CC(C)c1ccc(NC(=O)N2CC(C)(C)C2C(=O)O)cc1 ; COC(=O)c1ccc(-c2ccc(NC(=O)C3N(C(=O)Nc4ccc(C(C)C)cc4)CC3(C)C)cc2)cc1C]

A solution of methyl 4′-amino-3-methyl-[1,1′-biphenyl]-4-carboxylate (100 mg, 0.414 mmol), 1-methylimidazole (51.0 mg, 0.621 mmol), TCFH (174 mg, 0.621 mmol) and 1-((4-isopropylphenyl)carbamoyl)-3,3-dimethylazetidine-2-carboxylic acid (120 mg, 0.414 mmol) in MeCN (2.0 mL) was stirred at 25° C. for 1 hour. Upon completion, the reaction mixture was concentrated under reduced pressure. The crude residue was directly purified by silica gel column chromatography, eluted with petroleum ether/ethyl acetate (1:1), to afford methyl 4′-(1-((4-isopropylphenyl)carbamoyl)-3,3-dimethylazetidine-2-carboxamido)-3-methyl-[1,1′-biphenyl]-4-carboxylate (100 mg, 47%) as a yellow solid. LCMS (ESI) [M+H]⁺: 514.2.

### Step 3: 4′-(1-((4-isopropylphenyl)carbamoyl)-3,3-dimethylazetidine-2-carboxamido)-3-methyl-[1,1′-biphenyl]-4-carboxylic acid

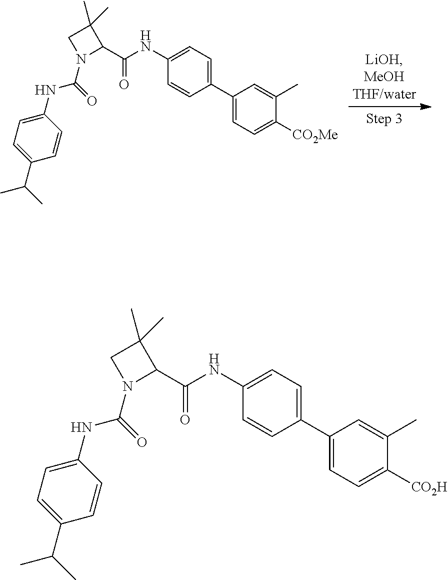 [SMILES: COC(=O)c1ccc(-c2ccc(NC(=O)C3N(C(=O)Nc4ccc(C(C)C)cc4)CC3(C)C)cc2)cc1C ; Cc1cc(-c2ccc(NC(=O)C3N(C(=O)Nc4ccc(C(C)C)cc4)CC3(C)C)cc2)ccc1C(=O)O]

A solution of methyl 4′-(1-((4-isopropylphenyl)carbamoyl)-3,3-dimethylazetidine-2-carboxamido)-3-methyl-[1,1′-biphenyl]-4-carboxylate (100 mg, 0.195 mmol) and lithium hydroxide monohydrate (40.9 mg, 0.975 mmol) in methanol (1.0 mL), THF (1.0 mL) and water (1.0 mL) was stirred at 25° C. for 4 hours. Upon completion, the reaction mixture was concentrated under reduced pressure. The crude residue was directly purified by reversed phase chromatography (conditions: column: C18 silica gel; mobile phase A: water (0.1% formic acid); mobile phase B: MeCN; gradient (B %): 0% to 100% over 20 min; detector: UV 254 nm) to afford 4′-(1-((4-isopropylphenyl)carbamoyl)-3,3-dimethylazetidine-2-carboxamido)-3-methyl-[1,1′-biphenyl]-4-carboxylic acid (30.2 mg, 31%) as a white solid. LCMS (ESI) [M+H]⁺: 500.2.

### Step 4: (S)-4′-(1-((4-isopropylphenyl)carbamoyl)-3,3-dimethylazetidine-2-carboxamido)-3-methyl-[1,1′-biphenyl]-4-carboxylic acid and (R)-4′-(1-((4-isopropylphenyl)carbamoyl)-3,3-dimethylazetidine-2-carboxamido)-3-methyl-[1,1′-biphenyl]-4-carboxylic acid

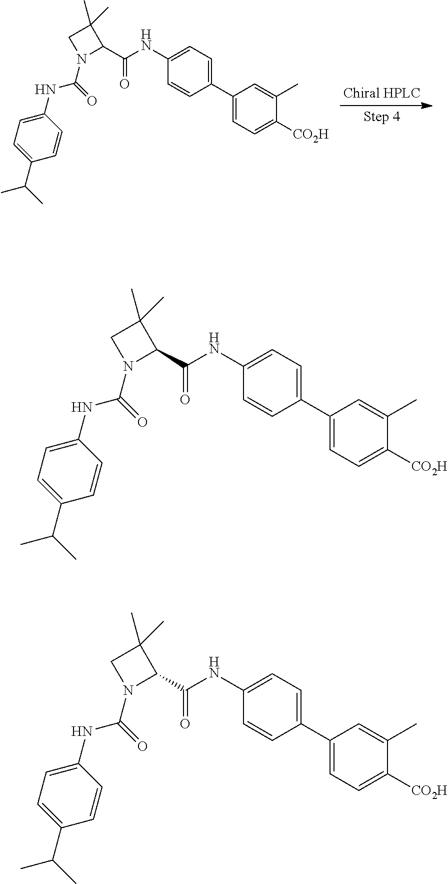 [SMILES: Cc1cc(-c2ccc(NC(=O)C3N(C(=O)Nc4ccc(C(C)C)cc4)CC3(C)C)cc2)ccc1C(=O)O ; Cc1cc(-c2ccc(NC(=O)[C@H]3N(C(=O)Nc4ccc(C(C)C)cc4)CC3(C)C)cc2)ccc1C(=O)O ; Cc1cc(-c2ccc(NC(=O)[C@@H]3N(C(=O)Nc4ccc(C(C)C)cc4)CC3(C)C)cc2)ccc1C(=O)O]

4′-(1-((4-isopropylphenyl)carbamoyl)-3,3-dimethylazetidine-2-carboxamido)-3-methyl-[1,1′-biphenyl]-4-carboxylic acid (30 mg) was resolved by preparative chiral HPLC (conditions: Column: CHIRAL ART Cellulose-SZ, 3*25 cm, 5 μm; mobile phase A: hexane (0.1% formic acid), mobile phase B: ethanol; flow rate: 40 mL/min; gradient (B %): isocratic, 30% B; detector: 220/254 nm; RT1 (min): 8.8; RT2 (min): 20.3) to afford (Compound 229a) (S)-4′-(1-((4-isopropylphenyl)carbamoyl)-3,3-dimethylazetidine-2-carboxamido)-3-methyl-[1,1′-biphenyl]-4-carboxylic acid (1^{st} peak, 9.3 mg, 30%; Method BM, Peak 1, 1.06 min) as a white solid and (Compound 229b) (R)-4′-(1-((4-isopropylphenyl)carbamoyl)-3,3-dimethylazetidine-2-carboxamido)-3-methyl-[1,1′-biphenyl]-4-carboxylic acid (2^{nd} peak, 7.2 mg, 23%; Method BM, Peak 2, 2.70 min) as a white solid.

| Cmpd # | Characterization Data |
|---|---|
| 229a | LCMS (ESI) [M + H]⁺: 501.3 |
| | ¹H NMR (400 MHz, methanol-d₄) δ 7.96 (d, J = 7.9 Hz, 1H), 7.76-7.69 (m, 2H), 7.69-7.61 (m, |
| | 2H), 7.56-7.46 (m, 2H), 7.36 (dd, J = 8.7, 2.1 Hz, 2H), 7.18-7.10 (m, 2H), 4.49 (s, 1H), 3.85 (d, |
| | J = 7.3 Hz, 1H), 3.78 (d, J = 7.3 Hz, 1H), 2.85 (p, J = 6.9 Hz, 1H), 2.64 (s, 3H), 1.47 (s, 3H), 1.27 |
| | (s, 3H), 1.22 (s, 3H), 1.21 (s, 3H). |
| 229b | LCMS (ESI) [M + H]⁺: 501.2 |
| | ¹H NMR (400 MHz, methanol-d₄) δ 7.98 (d, J = 8.0 Hz, 1H), 7.73 (dd, J = 8.7, 1.7 Hz, 2H), 7.69- |
| | 7.62 (m, 2H), 7.52 (dd, J = 10.9, 3.6 Hz, 2H), 7.40-7.32 (m, 2H), 7.14 (dd, J = 8.6, 1.7 Hz, 2H), |
| | 4.53-4.46 (m, 1H), 3.85 (d, J = 7.3 Hz, 1H), 3.78 (d, J = 7.3 Hz, 1H), 2.91-2.78 (m, 1H), 2.65 (s, |
| | 3H), 1.47 (d, J = 2.2 Hz, 3H), 1.27 (s, 3H), 1.22 (d, J = 1.4 Hz, 3H), 1.21 (d, J = 1.4 Hz, 3H). |

### Example 230: (S)-2-fluoro-4′-(1-((4-isopropylphenyl)carbamoyl)-3,3-dimethylazetidine-2-carboxamido)-[1,1′-biphenyl]-4-carboxylic acid and (R)-2-fluoro-4′-(1-((4-isopropylphenyl)carbamoyl)-3,3-dimethylazetidine-2-carboxamido)-[1,1′-biphenyl]-4-carboxylic acid

### Step 1: methyl 4′-amino-2-fluoro-[1,1′-biphenyl]-4-carboxylate

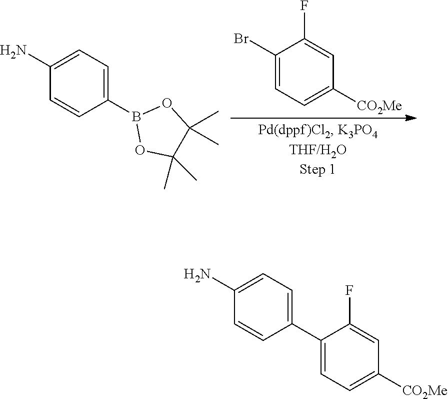 [SMILES: CC1(C)OB(c2ccc(N)cc2)OC1(C)C ; COC(=O)c1ccc(Br)c(F)c1 ; COC(=O)c1ccc(-c2ccc(N)cc2)c(F)c1]

A solution of methyl 4-bromo-3-fluorobenzoate (500 mg, 2.14 mmol), 4-(4,4,5,5-tetramethyl-1,3,2-dioxaborolan-2-yl)aniline (517.0 mg, 2.36 mmol), Pd(dppf)Cl₂ (175 mg, 0.230 mmol), tribasic potassium phosphate (1.30 g, 6.43 mmol) in THF (5.0 mL) and water (0.5 mL) was heated to 80° C. for 2 hours. Upon completion, the reaction mixture was concentrated under reduced pressure. The crude residue was directly purified by silica gel column chromatography, eluted with petroleum ether/ethyl acetate (1:1) to afford methyl 4′-amino-2-fluoro-[1,1′-biphenyl]-4-carboxylate (400 mg, 76%) as a pale-yellow solid. LCMS (ESI) [M+H]⁺: 246.1.

### Step 2: Methyl 2-fluoro-4′-(1-((4-isopropylphenyl)carbamoyl)-3,3-dimethylazetidine-2-carboxamido)-[1,1′-biphenyl]-4-carboxylate

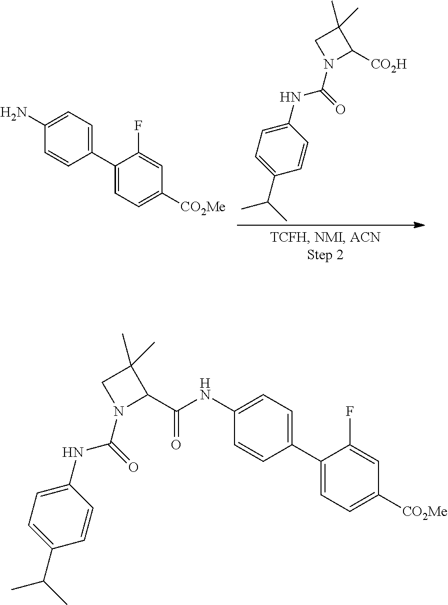 [SMILES: COC(=O)c1ccc(-c2ccc(N)cc2)c(F)c1 ; CC(C)c1ccc(NC(=O)N2CC(C)(C)C2C(=O)O)cc1 ; COC(=O)c1ccc(-c2ccc(NC(=O)C3N(C(=O)Nc4ccc(C(C)C)cc4)CC3(C)C)cc2)c(F)c1]

A solution of 1-[(4-isopropylphenyl)carbamoyl]-3,3-dimethylazetidine-2-carboxylic acid (50.0 mg, 0.17 mmol), 1-methylimidazole (42.4 mg, 0.510 mmol), methyl 4′-amino-2-fluoro-[1,1′-biphenyl]-4-carboxylate (46.4 mg, 0.180 mmol) and TCFH (72.4 mg, 0.250 mmol) in DMF (2.0 mL) was stirred at room temperature for 2 hours. Upon completion, the reaction mixture was concentrated under reduced pressure. The crude residue was directly purified by silica gel column chromatography, eluted with petroleum ether/ethyl acetate (1:1), to afford methyl 2-fluoro-4′-(1-((4-isopropylphenyl)carbamoyl)-3,3-dimethylazetidine-2-carboxamido)-[1,1′-biphenyl]-4-carboxylate (60 mg, 67%) as an off-white solid. LCMS (ESI) [M+H]⁺: 518.3.

### Step 3: 2-fluoro-4′-(1-((4-isopropylphenyl)carbamoyl)-3,3-dimethylazetidine-2-carboxamido)-[1,1′-biphenyl]-4-carboxylic acid

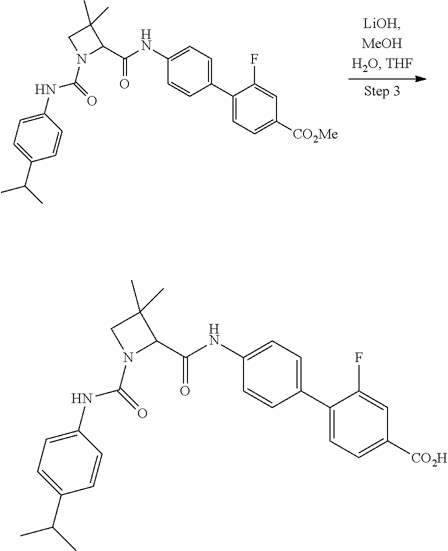 [SMILES: COC(=O)c1ccc(-c2ccc(NC(=O)C3N(C(=O)Nc4ccc(C(C)C)cc4)CC3(C)C)cc2)c(F)c1 ; CC(C)c1ccc(NC(=O)N2CC(C)(C)C2C(=O)Nc2ccc(-c3ccc(C(=O)O)cc3F)cc2)cc1]

A solution of methyl 2-fluoro-4′-(1-((4-isopropylphenyl)carbamoyl)-3,3-dimethylazetidine-2-carboxamido)-[1,1′-biphenyl]-4-carboxylate (55.0 mg, 0.10 mmol) and lithium hydroxide monohydrate (89.0 mg, 2.12 mmol) in methanol (1.0 mL), THF (1.0 mL) and water (1.0 mL) was stirred at 25° C. for 4 hours. Upon completion, the reaction mixture was concentrated under reduced pressure. The crude residue was directly purified by reversed phase chromatography (conditions: column: C18 silica gel; mobile phase A: water (0.1% formic acid); mobile phase B: MeCN; gradient (B %): 0% to 100% over 20 min; detector: UV 254 nm) to afford 2-fluoro-4′-(1-((4-isopropylphenyl)carbamoyl)-3,3-dimethylazetidine-2-carboxamido)-[1,1′-biphenyl]-4-carboxylic acid (45.0 mg, 84%) as a white solid. LCMS (ESI) [M+H]⁺: 504.2.

### Step 4: (S)-2-fluoro-4′-(1-((4-isopropylphenyl)carbamoyl)-3,3-dimethylazetidine-2-carboxamido)-[1,1′-biphenyl]-4-carboxylic acid and (R)-2-fluoro-4′-(1-((4-isopropylphenyl)carbamoyl)-3,3-dimethylazetidine-2-carboxamido)-[1,1′-biphenyl]-4-carboxylic acid

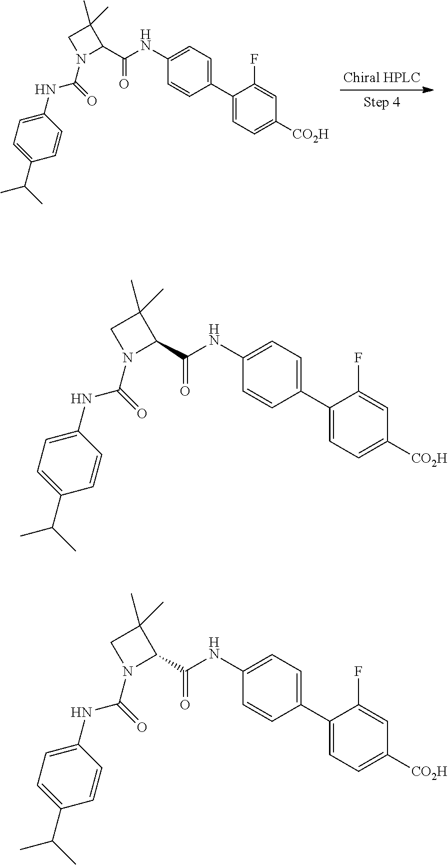 [SMILES: CC(C)c1ccc(NC(=O)N2CC(C)(C)C2C(=O)Nc2ccc(-c3ccc(C(=O)O)cc3F)cc2)cc1 ; CC(C)c1ccc(NC(=O)N2CC(C)(C)[C@H]2C(=O)Nc2ccc(-c3ccc(C(=O)O)cc3F)cc2)cc1 ; CC(C)c1ccc(NC(=O)N2CC(C)(C)[C@@H]2C(=O)Nc2ccc(-c3ccc(C(=O)O)cc3F)cc2)cc1]

2-fluoro-4′-(1-((4-isopropylphenyl)carbamoyl)-3,3-dimethylazetidine-2-carboxamido)-[1,1′-biphenyl]-4-carboxylic acid (45 mg) was resolved by preparative chiral HPLC (conditions: Column: CHIRAL ART Cellulose-SZ, 3*25 cm, 5 μm; mobile phase A: hexane (0.1% formic acid), mobile phase B: ethanol; flow rate: 40 mL/min; gradient (B %): isocratic, 50% B; detector: 220/254 nm; RT1 (min): 5.5; RT2 (min): 10.3) to afford (Compound 230a) (S)-2-fluoro-4′-(1-((4-isopropylphenyl)carbamoyl)-3,3-dimethylazetidine-2-carboxamido)-[1,1′-biphenyl]-4-carboxylic acid (1^{st} peak, 12.3 mg, 32%; Method BE, Peak 1, 0.73 min) as a white solid and (Compound 230b) (R)-2-fluoro-4′-(1-((4-isopropylphenyl)carbamoyl)-3,3-dimethylazetidine-2-carboxamido)-[1,1′-biphenyl]-4-carboxylic acid (2^{nd} peak, 11.7 mg, 30%; Method BE, Peak 2, 2.24 min) as a white solid.

| Cmpd # | Characterization Data |
|---|---|
| 230a | LCMS (ESI) [M + H]⁺: 504.2 |
| | ¹H NMR (400 MHz, methanol-d₄) δ 7.91-7.86 (m, 1H), 7.80-7.71 (m, 3H), 7.65-7.54 (m, 3H), |
| | 7.36 (d, J = 8.6 Hz, 2H), 7.14 (d, J = 8.6 Hz, 2H), 4.49 (s, 1H), 3.85 (d, J = 7.3 Hz, 1H), 3.78 (d, J = |
| | 7.4 Hz, 1H), 2.85 (p, J = 6.9 Hz, 1H), 1.47 (s, 3H), 1.27 (s, 3H), 1.23 (s, 3H), 1.21 (s, 3H). |
| 230b | LCMS (ESI) [M + H]⁺: 504.2 |
| | ¹H NMR (400 MHz, methanol-d₄) δ 7.89 (d, J = 8.1 Hz, 1H), 7.81-7.70 (m, 3H), 7.60 (t, J = 9.1 |
| | Hz, 3H), 7.36 (d, J = 8.3 Hz, 2H), 7.15 (d, J = 8.5 Hz, 2H), 4.49 (s, 1H), 3.85 (d, J = 7.4 Hz, 1H), |
| | 3.78 (d, J = 7.4 Hz, 1H), 2.85 (p, J = 6.9 Hz, 1H), 1.47 (s, 3H), 1.27 (s, 3H), 1.23 (s, 3H), 1.21 (s, |
| | 3H). |

### Example 231: (S)-4-(5-(1-((4-isopropylphenyl)carbamoyl)-3,3-dimethylazetidine-2-carboxamido)pyridin-2-yl)-3-methylbenzoic acid and (R)-4-(5-(1-((4-isopropylphenyl)carbamoyl)-3,3-dimethylazetidine-2-carboxamido)pyridin-2-yl)-3-methylbenzoic acid

### Step 1: methyl 4-(5-aminopyridin-2-yl)-3-methylbenzoate

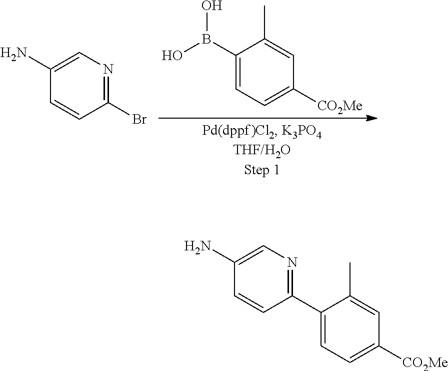 [SMILES: Nc1ccc(Br)nc1 ; COC(=O)c1ccc(B(O)O)c(C)c1 ; COC(=O)c1ccc(-c2ccc(N)cn2)c(C)c1]

A mixture of 4-(methoxycarbonyl)-2-methylphenylboronic acid (336 mg, 1.73 mmol), 6-bromopyridin-3-amine (300 mg, 1.73 mmol), tribasic potassium phosphate (1.1 g, 5.20 mmol) and Pd(dppf)Cl₂ (127 mg, 0.173 mmol) and in THF (5.0 mL) and water (1.0 mL) was stirred at 60° C. for 1 hour. Upon completion, the reaction mixture was concentrated under reduced pressure. The crude residue was directly purified by silica gel column chromatography, eluted with petroleum ether/ethyl acetate (1:1), to afford methyl 4-(5-aminopyridin-2-yl)-3-methylbenzoate (200 mg, 48%) as a yellow solid. LCMS (ESI) [M+H]⁺: 243.1.

### Step 2: methyl 4-(5-(1-((4-isopropylphenyl)carbamoyl)-3,3-dimethylazetidine-2-carboxamido)pyridin-2-yl)-3-methylbenzoate

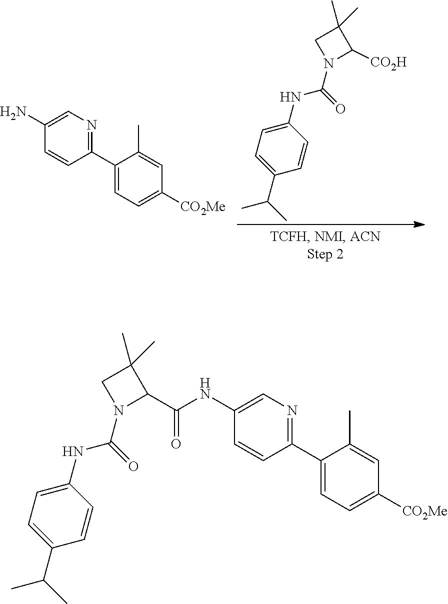 [SMILES: COC(=O)c1ccc(-c2ccc(N)cn2)c(C)c1 ; CC(C)c1ccc(NC(=O)N2CC(C)(C)C2C(=O)O)cc1 ; COC(=O)c1ccc(-c2ccc(NC(=O)C3N(C(=O)Nc4ccc(C(C)C)cc4)CC3(C)C)cn2)c(C)c1]

A solution of 1-[(4-isopropylphenyl)carbamoyl]-3,3-dimethylazetidine-2-carboxylic acid (50 mg, 0.172 mmol), 1-methylimidazole (70.7 mg, 0.860 mmol), methyl 4-(5-aminopyridin-2-yl)-3-methylbenzoate (41.7 mg, 0.172 mmol) and TCFH (96.6 mg, 0.344 mmol) in DMF (2.0 mL) was stirred at room temperature for 2 hours. Upon completion, the reaction mixture was concentrated under reduced pressure. The crude residue was directly purified by silica gel column chromatography, eluted with petroleum ether/ethyl acetate (1:1) to afford methyl 4-(5-(1-((4-isopropylphenyl)carbamoyl)-3,3-dimethylazetidine-2-carboxamido)pyridin-2-yl)-3-methylbenzoate (70 mg, 79%) as an off-white solid. LCMS (ESI) [M+H]⁺: 515.3.

### Step 3: 4-(5-(1-((4-isopropylphenyl)carbamoyl)-3,3-dimethylazetidine-2-carboxamido)pyridin-2-yl)-3-methylbenzoic acid

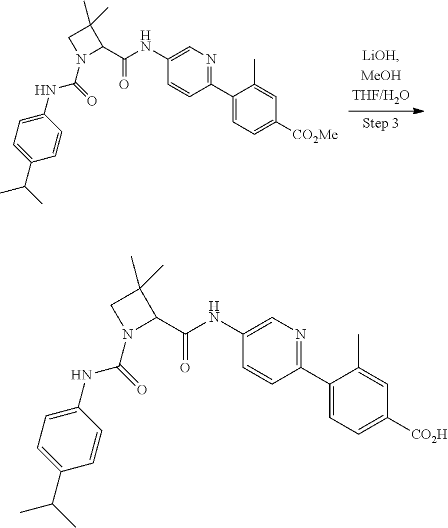 [SMILES: COC(=O)c1ccc(-c2ccc(NC(=O)C3N(C(=O)Nc4ccc(C(C)C)cc4)CC3(C)C)cn2)c(C)c1 ; Cc1cc(C(=O)O)ccc1-c1ccc(NC(=O)C2N(C(=O)Nc3ccc(C(C)C)cc3)CC2(C)C)cn1]

A solution of methyl 4-(5-(1-((4-isopropylphenyl)carbamoyl)-3,3-dimethylazetidine-2-carboxamido)pyridin-2-yl)-3-methylbenzoate (70 mg, 0.136 mmol) and lithium hydroxide monohydrate (41.0 mg, 0.970 mmol) in methanol (1.0 mL), THF (1.0 mL), and water (1.0 mL) was stirred at 25° C. for 4 hours. Upon completion, the mixture was concentrated under reduced pressure. The crude residue was directly purified by reversed phase chromatography (conditions: column: C18 silica gel; mobile phase A: water (0.1% formic acid); mobile phase B: MeCN; gradient (B %): 0% to 100% over 20 min; detector: UV 254 nm) to afford 4-(5-(1-((4-isopropylphenyl)carbamoyl)-3,3-dimethylazetidine-2-carboxamido)pyridin-2-yl)-3-methylbenzoic acid (60.0 mg, 85%) as a white solid. LCMS (ESI) [M+H]⁺: 501.2.

### Step 4: (S)-4-(5-(1-((4-isopropylphenyl)carbamoyl)-3,3-dimethylazetidine-2-carboxamido)pyridin-2-yl)-3-methylbenzoic acid and (R)-4-(5-(1-((4-isopropylphenyl)carbamoyl)-3,3-dimethylazetidine-2-carboxamido)pyridin-2-yl)-3-methylbenzoic acid

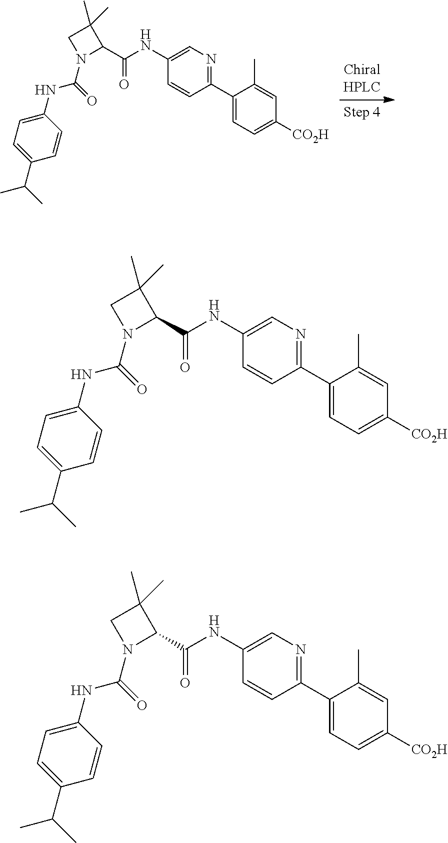 [SMILES: Cc1cc(C(=O)O)ccc1-c1ccc(NC(=O)C2N(C(=O)Nc3ccc(C(C)C)cc3)CC2(C)C)cn1 ; Cc1cc(C(=O)O)ccc1-c1ccc(NC(=O)[C@H]2N(C(=O)Nc3ccc(C(C)C)cc3)CC2(C)C)cn1 ; Cc1cc(C(=O)O)ccc1-c1ccc(NC(=O)[C@@H]2N(C(=O)Nc3ccc(C(C)C)cc3)CC2(C)C)cn1]

4-(5-(1-((4-isopropylphenyl)carbamoyl)-3,3-dimethylazetidine-2-carboxamido)pyridin-2-yl)-3-methylbenzoic acid (60 mg) was resolved by preparative chiral HPLC (conditions: Column: CHIRAL ART Cellulose-SZ, 3*25 cm, 5 μm; mobile phase A: hexane (0.1% formic acid), mobile phase B: ethanol; flow rate: 40 mL/min; gradient (B %): isocratic, 50% B; detector: 220/254 nm; RT1 (min): 5.4; RT2 (min): 8.5) to afford (Compound 231a) (S)-4-(5-(1-((4-isopropylphenyl)carbamoyl)-3,3-dimethylazetidine-2-carboxamido)pyridin-2-yl)-3-methylbenzoic acid (1^{st} peak, 28.6 mg, 47%; Method BE, Peak 1, 0.60 min) as a white solid and (Compound 231b) (R)-4-(5-(1-((4-isopropylphenyl)carbamoyl)-3,3-dimethylazetidine-2-carboxamido)pyridin-2-yl)-3-methylbenzoic acid (2^{nd} peak, 26.5 mg, 44%; Method BE, Peak 2, 0.96 min) as a white solid.

| Cmpd # | Characterization Data |
|---|---|
| 231a | LCMS (ESI) [M + H]⁺: 501.3 |
| | ¹H NMR (400 MHz, methanol-d₄) δ 8.89 (dd, J = 2.5, 0.7 Hz, 1H), 8.28 (dd, J = 8.5, 2.6 Hz, 1H), |
| | 7.97 (d, J = 1.7 Hz, 1H), 7.92 (dd, J = 8.0, 1.7 Hz, 1H), 7.52 (dd, J = 8.5, 0.9 Hz, 1H), 7.45 (d, J = |
| | 7.9 Hz, 1H), 7.36 (d, J = 8.6 Hz, 2H), 7.14 (d, J = 8.6 Hz, 2H), 4.52 (s, 1H), 3.86 (d, J = 7.4 |
| | Hz, 1H), 3.80 (d, J = 7.3 Hz, 1H), 2.85 (p, J = 6.9 Hz, 1H), 2.36 (s, 3H), 1.48 (s, 3H), 1.28 (s, |
| | 3H), 1.22 (s, 3H), 1.21 (s, 3H). |
| 231b | LCMS (ESI) [M + H]⁺: 501.3 |
| | ¹H NMR (400 MHz, methanol-d₄) δ 8.89 (dd, J = 2.6, 0.7 Hz, 1H), 8.28 (dd, J = 8.5, 2.6 Hz, 1H), |
| | 7.97 (d, J = 1.8 Hz, 1H), 7.92 (dd, J = 8.0, 1.8 Hz, 1H), 7.53 (dd, J = 8.5, 0.7 Hz, 1H), 7.45 (d, J = |
| | 8.0 Hz, 1H), 7.36 (d, J = 8.5 Hz, 2H), 7.14 (d, J = 8.6 Hz, 2H), 4.51 (s, 1H), 3.86 (d, J = 7.3 |
| | Hz, 1H), 3.80 (d, J = 7.3 Hz, 1H), 2.85 (p, J = 6.9 Hz, 1H), 2.36 (s, 3H), 1.48 (s, 3H), 1.28 (s, |
| | 3H), 1.22 (s, 3H), 1.21 (s, 3H). |

### Example 232: (S)-5-(3-fluoro-4-(1-((4-isopropylphenyl)carbamoyl)-3,3-dimethylazetidine-2-carboxamido)phenyl)picolinic acid and (R)-5-(3-fluoro-4-(1-((4-isopropylphenyl)carbamoyl)-3,3-dimethylazetidine-2-carboxamido)phenyl)picolinic acid

### Step 1: methyl 5-(4-amino-3-fluorophenyl)picolinate

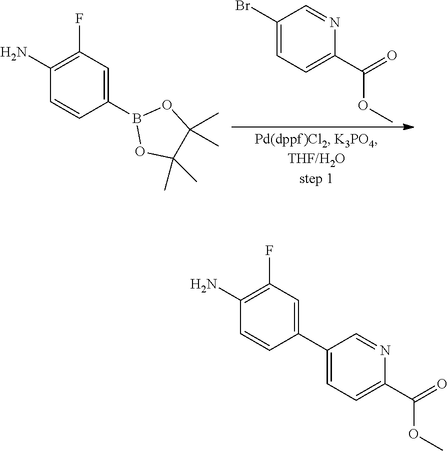 [SMILES: CC1(C)OB(c2ccc(N)c(F)c2)OC1(C)C ; COC(=O)c1ccc(Br)cn1 ; COC(=O)c1ccc(-c2ccc(N)c(F)c2)cn1]

Into a solution of methyl 5-bromopyridine-2-carboxylate (364 mg, 1.68 mmol), 2-fluoro-4-(4,4,5,5-tetramethyl-1,3,2-dioxaborolan-2-yl)aniline (400 mg, 1.68 mmol) and Pd(dppf)Cl₂ (123 mg, 0.169 mmol) in 1,4-dioxane (10 mL) was added a solution of K₂CO₃ (699 mg, 5.06 mmol) in H₂O (2 mL). The mixture was stirred at 80° C. for 2 hours. Upon completion, the mixture was concentrated under reduced pressure. The crude residue was directly purified by silica gel column chromatography, eluted with petroleum ether/ethyl acetate (1:1) to afford methyl 5-(4-amino-3-fluorophenyl)picolinate (400 mg, 96%) as a white solid. LCMS (ESI) [M+H]⁺: 247.

### Step 2: methyl 5-(3-fluoro-4-(1-((4-isopropylphenyl)carbamoyl)-3,3-dimethylazetidine-2-carboxamido)phenyl)picolinate

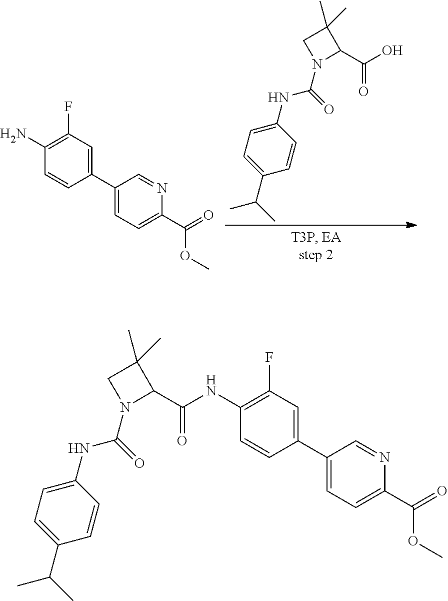

Propanephosphonic acid cyclic anhydride (0.18 mL, 0.234 mmol, 50% in ethyl acetate) was added into a solution of methyl 5-(4-amino-3-fluorophenyl)picolinate (50.0 mg, 0.203 mmol), and 1-[(4-isopropylphenyl)carbamoyl]-3,3-dimethylazetidine-2-carboxylic acid (59.0 mg, 0.203 mmol) in EtOAc (0.6 mL) and the mixture was stirred at 50° C. for 30 minutes. Upon completion, the resulting mixture was diluted with EtOAc (20 mL). The organic layer was washed with H₂O (3×20 mL) and concentrated under reduced pressure. The crude residue was purified by silica gel column chromatography, eluted with petroleum ether/ethyl acetate (1:1) to afford methyl 5-(3-fluoro-4-(1-((4-isopropylphenyl)carbamoyl)-3,3-dimethylazetidine-2-carboxamido)phenyl)picolinate (68 mg, 64%) as a white solid. LCMS (ESI) [M+H]⁺: 519.

### Step 3: 5-(3-fluoro-4-(1-((4-isopropylphenyl)carbamoyl)-3,3-dimethylazetidine-2-carboxamido)phenyl)picolinic acid

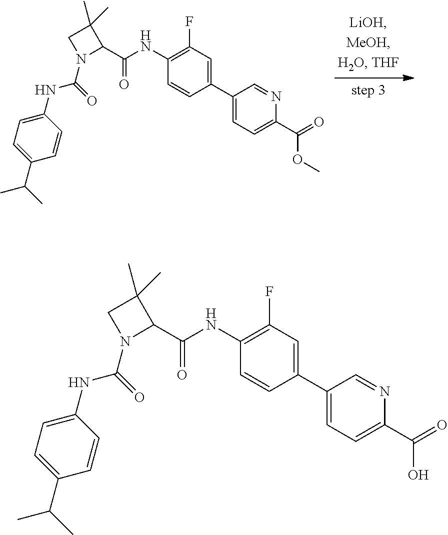

A solution of methyl 5-(3-fluoro-4-(1-((4-isopropylphenyl)carbamoyl)-3,3-dimethylazetidine-2-carboxamido)phenyl)picolinate (68 mg, 0.131 mmol) and lithium hydroxide monohydrate (27.5 mg, 0.655 mmol) in MeOH (1 mL), THF (1 mL) and H₂O (1 mL) was stirred at 25° C. for 4 hours. Upon completion, the reaction mixture was concentrated under reduced pressure. The crude residue was directly purified by reverse phase flash chromatography (conditions: column: C18 silica gel; mobile phase A: water (0.1% formic acid); mobile phase B: MeCN; gradient (B %): 0% to 100% over 20 min; detector: UV 254 nm) to afford 5-(3-fluoro-4-(1-((4-isopropylphenyl)carbamoyl)-3,3-dimethylazetidine-2-carboxamido)phenyl)picolinic acid (40 mg, 60%) as a white solid. LCMS (ESI) [M+H]⁺: 505.

### Step 4: (S)-5-(3-fluoro-4-(1-((4-isopropylphenyl)carbamoyl)-3,3-dimethylazetidine-2-carboxamido)phenyl)picolinic acid and (R)-5-(3-fluoro-4-(1-((4-isopropylphenyl)carbamoyl)-3,3-dimethylazetidine-2-carboxamido)phenyl)picolinic acid

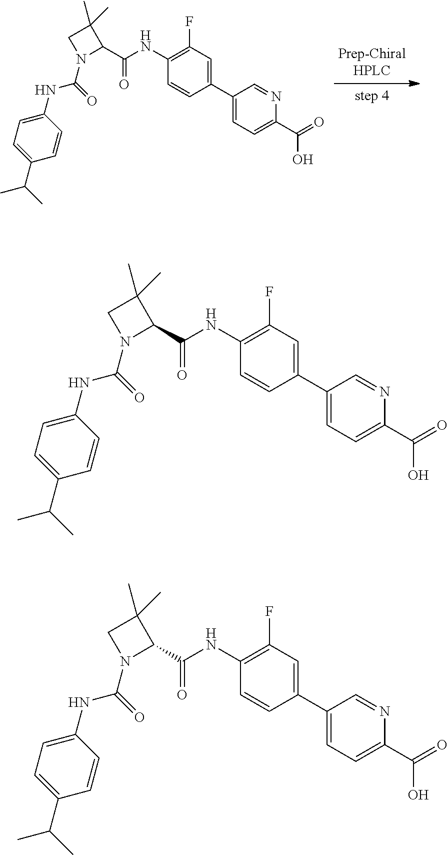 [SMILES: CC(C)c1ccc(NC(=O)N2CC(C)(C)C2C(=O)Nc2ccc(-c3ccc(C(=O)O)nc3)cc2F)cc1 ; CC(C)c1ccc(NC(=O)N2CC(C)(C)[C@H]2C(=O)Nc2ccc(-c3ccc(C(=O)O)nc3)cc2F)cc1 ; CC(C)c1ccc(NC(=O)N2CC(C)(C)[C@@H]2C(=O)Nc2ccc(-c3ccc(C(=O)O)nc3)cc2F)cc1]

5-(3-fluoro-4-(1-((4-isopropylphenyl)carbamoyl)-3,3-dimethylazetidine-2-carboxamido)phenyl)picolinic acid (40 mg) was resolved by preparative chiral HPLC (conditions: Column: CHIRAL ART Cellulose-SZ 3*25 cm, 5 μm; Mobile Phase A: MTBE (0.1% formic acid), Mobile Phase B: MEOH; Flow rate: 40 mL/min; Gradient (B %): isocratic, 50% B; Wave Length: 220/254 nm; RT1 (min): 8; RT2 (min): 19) to afford (Compound 232a) (S)-5-(3-fluoro-4-(1-((4-isopropylphenyl)carbamoyl)-3,3-dimethylazetidine-2-carboxamido)phenyl)picolinic acid (1^{st} peak, 13.0 mg, 43%; Method BL, Peak 1, 0.87 min) as a white solid and (Compound 232b) (R)-5-(3-fluoro-4-(1-((4-isopropylphenyl)carbamoyl)-3,3-dimethylazetidine-2-carboxamido)phenyl)picolinic acid (2^{nd} peak, 14.1 mg, 47%; Method BL, Peak 2, 2.40 min) as a white solid.

| Cmpd # | Characterization Data |
|---|---|
| 232a | LCMS (ESI) [M + H]⁺: 505.2 |
| | ¹H NMR (400 MHz, methanol-d₄) δ 8.99-8.88 (m, 1H), 8.32-8.19 (m, 2H), 8.16-8.10 (m, 1H), |
| | 7.71-7.52 (m, 2H), 7.36 (d, J = 8.6 Hz, 2H), 7.15 (d, J = 7.8 Hz, 2H), 4.56 (s, 1H), 3.90-3.74 |
| | (m, 2H), 2.85 (p, J = 7.0 Hz, 1H), 1.46 (s, 3H), 1.30 (s, 3H), 1.23 (d, J = 0.9 Hz, 3H), 1.21 (d, J = |
| | 0.9 Hz, 3H). |
| 232b | LCMS (ESI) [M + H]⁺: 505.3 |
| | ¹H NMR (400 MHz, methanol-d₄) δ 9.00-8.83 (m, 1H), 8.30-8.17 (m, 2H), 8.17-8.06 (m, 1H), |
| | 7.68-7.48 (m, 2H), 7.36 (d, J = 8.5 Hz, 2H), 7.20-7.07 (m, 2H), 4.56 (s, 1H), 3.87-3.76 (m, |
| | 2H), 2.85 (dt, J = 6.9, 3.4 Hz, 1H), 1.46 (s, 3H), 1.29 (s, 3H), 1.23 (d, J = 2.2 Hz, 3H), 1.21 (d, J = |
| | 2.3 Hz, 3H). |

### Example 233: (S)-5-(4-(1-((4-isopropylphenyl)carbamoyl)-3,3-dimethylazetidine-2-carboxamido)phenyl)-6-methoxypicolinic acid and (R)-5-(4-(1-((4-isopropylphenyl)carbamoyl)-3,3-dimethylazetidine-2-carboxamido)phenyl)-6-methoxypicolinic acid

### Step 1: Methyl 5-(4-aminophenyl)-6-methoxypicolinate

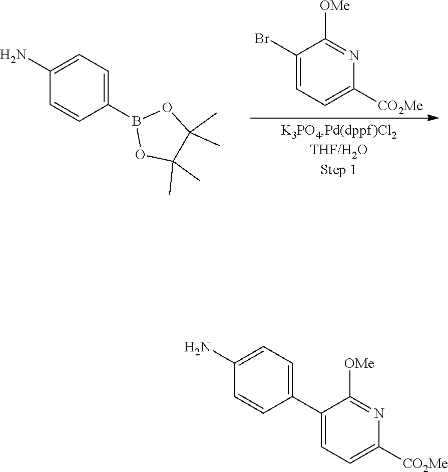 [SMILES: CC1(C)OB(c2ccc(N)cc2)OC1(C)C ; COC(=O)c1ccc(Br)c(OC)n1 ; COC(=O)c1ccc(-c2ccc(N)cc2)c(OC)n1]

A solution of 4-(4,4,5,5-tetramethyl-1,3,2-dioxaborolan-2-yl)aniline (200 mg, 0.913 mmol), methyl 5-bromo-6-methoxypyridine-2-carboxylate (224 mg, 0.913 mmol), tribasic potassium phosphate (581 mg, 2.74 mmol), and Pd(dppf)Cl₂ (66 mg, 0.091 mmol) in THF (4.0 mL) and water (0.20 mL) was heated at 80° C. for 3 hours. Upon completion, the reaction mixture was concentrated under reduced pressure. The crude residue was directly purified by silica gel column chromatography, eluted with petroleum ether/ethyl acetate (4:1) to afford methyl 5-(4-aminophenyl)-6-methoxypyridine-2-carboxylate (160 mg, 67%) as a white solid. LCMS (ESI) [M+H]⁺: 259.0.

### Step 2: methyl 5-(4-(1-((4-isopropylphenyl)carbamoyl)-3,3-dimethylazetidine-2-carboxamido)phenyl)-6-methoxypicolinate

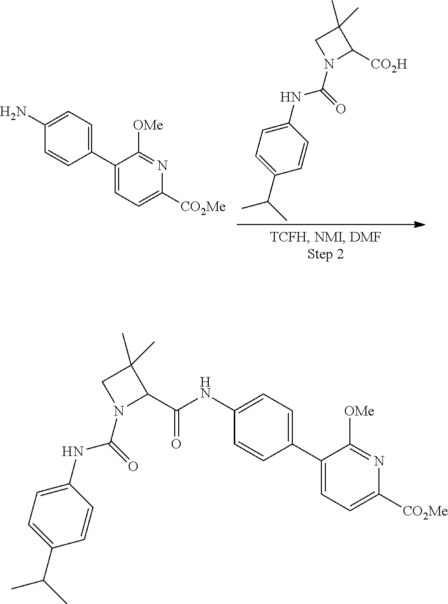 [SMILES: COC(=O)c1ccc(-c2ccc(N)cc2)c(OC)n1 ; CC(C)c1ccc(NC(=O)N2CC(C)(C)C2C(=O)O)cc1 ; COC(=O)c1ccc(-c2ccc(NC(=O)C3N(C(=O)Nc4ccc(C(C)C)cc4)CC3(C)C)cc2)c(OC)n1]

A solution of 1-[(4-isopropylphenyl)carbamoyl]-3,3-dimethylazetidine-2-carboxylic acid (179 mg, 0.619 mmol), 1-methylimidazole (152 mg, 1.86 mmol), methyl 5-(4-aminophenyl)-6-methoxypyridine-2-carboxylate (41.7 mg, 0.172 mmol) and TCFH (225 mg, 0.805 mmol) in DMF (4.0 mL) was stirred at room temperature for 2 hours. Upon completion, the reaction mixture was concentrated under reduced pressure. The crude residue was directly purified by silica gel column chromatography, eluted with petroleum ether/ethyl acetate (1:1), to afford methyl 5-(4-(1-((4-isopropylphenyl)carbamoyl)-3,3-dimethylazetidine-2-carboxamido)phenyl)-6-methoxypicolinate (120 mg, 36%) as an off-white solid. LCMS (ESI) [M+H]⁺: 531.2.

### Step 3: 5-(4-(1-((4-isopropylphenyl)carbamoyl)-3,3-dimethylazetidine-2-carboxamido)phenyl)-6-methoxypicolinic acid

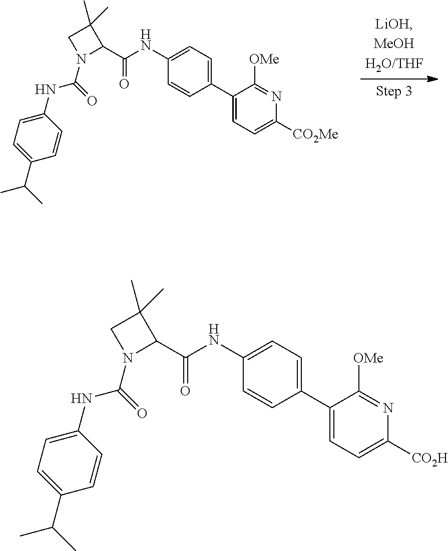 [SMILES: COC(=O)c1ccc(-c2ccc(NC(=O)C3N(C(=O)Nc4ccc(C(C)C)cc4)CC3(C)C)cc2)c(OC)n1 ; COc1nc(C(=O)O)ccc1-c1ccc(NC(=O)C2N(C(=O)Nc3ccc(C(C)C)cc3)CC2(C)C)cc1]

A solution of methyl 5-(4-(1-((4-isopropylphenyl)carbamoyl)-3,3-dimethylazetidine-2-carboxamido)phenyl)-6-methoxypicolinate (120 mg, 0.226 mmol) and lithium hydroxide monohydrate (47.5 mg, 1.13 mmol) in methanol (1.0 mL), THF (1.0 mL), and water (1.0 mL) was stirred at 25° C. for 4 hours. Upon completion, the mixture was concentrated under reduced pressure. The crude residue was directly purified by reversed phase chromatography (conditions: column: C18 silica gel; mobile phase A: water (0.1% formic acid); mobile phase B: MeCN; gradient (B %): 0% to 100% over 20 min; detector: UV 254 nm) to afford 5-(4-(1-((4-isopropylphenyl)carbamoyl)-3,3-dimethylazetidine-2-carboxamido)phenyl)-6-methoxypicolinic acid (60 mg, 51%) as a white solid. LCMS (ESI) [M+H]⁺: 517.2.

### Step 4: (S)-5-(4-(1-((4-isopropylphenyl)carbamoyl)-3,3-dimethylazetidine-2-carboxamido)phenyl)-6-methoxypicolinic acid and (R)-5-(4-(1-((4-isopropylphenyl)carbamoyl)-3,3-dimethylazetidine-2-carboxamido)phenyl)-6-methoxypicolinic acid

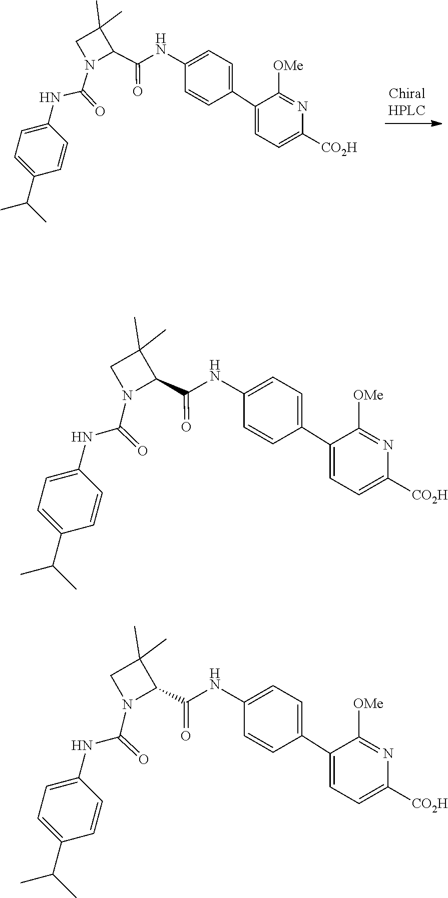 [SMILES: COc1nc(C(=O)O)ccc1-c1ccc(NC(=O)C2N(C(=O)Nc3ccc(C(C)C)cc3)CC2(C)C)cc1 ; COc1nc(C(=O)O)ccc1-c1ccc(NC(=O)[C@H]2N(C(=O)Nc3ccc(C(C)C)cc3)CC2(C)C)cc1 ; COc1nc(C(=O)O)ccc1-c1ccc(NC(=O)[C@@H]2N(C(=O)Nc3ccc(C(C)C)cc3)CC2(C)C)cc1]

5-(4-(1-((4-isopropylphenyl)carbamoyl)-3,3-dimethylazetidine-2-carboxamido)phenyl)-6-methoxypicolinic acid (60 mg) was resolved by preparative chiral HPLC (conditions: Column: CHIRAL ART Cellulose-SZ, 3*25 cm, 5 μm; mobile phase A: hexane (0.1% formic acid), mobile phase B: ethanol; flow rate: 40 mL/min; gradient (B %): isocratic, 50% B; detector: 220/254 nm; RT1 (min): 5.4; RT2 (min): 8.5) to afford (Compound 233a) (S)-5-(4-(1-((4-isopropylphenyl)carbamoyl)-3,3-dimethylazetidine-2-carboxamido)phenyl)-6-methoxypicolinic acid (1^{st} peak, 29.8 mg, 49%; Method BL, Peak 1, 0.67 min) as a white solid and (Compound 233b) (R)-5-(4-(1-((4-isopropylphenyl)carbamoyl)-3,3-dimethylazetidine-2-carboxamido)phenyl)-6-methoxypicolinic acid (2^{nd} peak, 23.3 mg, 38%; Method BL, Peak 2, 1.93 min) as a white solid.

| Cmpd # | Characterization Data |
|---|---|
| 233a | LCMS (ESI) [M + H]⁺: 517.3 |
| | ¹H NMR (400 MHz, methanol-d₄) δ 7.88-7.77 (m, 2H), 7.70 (d, J = 8.8 Hz, 2H), 7.60 (d, J = |
| | 8.7 Hz, 2H), 7.35 (d, J = 8.5 Hz, 2H), 7.14 (d, J = 8.5 Hz, 2H), 4.48 (s, 1H), 4.04 (s, 3H), 3.85 (d, |
| | J = 7.3 Hz, 1H), 3.78 (d, J = 7.3 Hz, 1H), 2.85 (p, J = 6.9 Hz, 1H), 1.47 (s, 3H), 1.27 (s, 3H), |
| | 1.23 (s, 3H), 1.21 (s, 3H). |
| 233b | LCMS (ESI) [M + H]⁺: 517.3 |
| | ¹H NMR (400 MHz, methanol-d₄) δ 7.89-7.79 (m, 2H), 7.74-7.68 (m, 2H), 7.63-7.57 (m, |
| | 2H), 7.39-7.33 (m, 2H), 7.18-7.11 (m, 2H), 4.48 (s, 1H), 4.04 (s, 3H), 3.85 (d, J = 7.3 Hz, 1H), |
| | 3.78 (d, J = 7.3 Hz, 1H), 2.85 (p, J = 6.9 Hz, 1H), 1.47 (s, 3H), 1.27 (s, 3H), 1.23 (s, 3H), 1.21 (s, |
| | 3H). |

### Example 234: (S)-6′-(1-((4-isopropylphenyl)carbamoyl)-3,3-dimethylazetidine-2-carboxamido)-[3,3′-bipyridine]-6-carboxylic acid and (R)-6′-(1-((4-isopropylphenyl)carbamoyl)-3,3-dimethylazetidine-2-carboxamido)-[3,3′-bipyridine]-6-carboxylic acid

### Step 1: methyl 6′-amino-[3,3′-bipyridine]-6-carboxylate

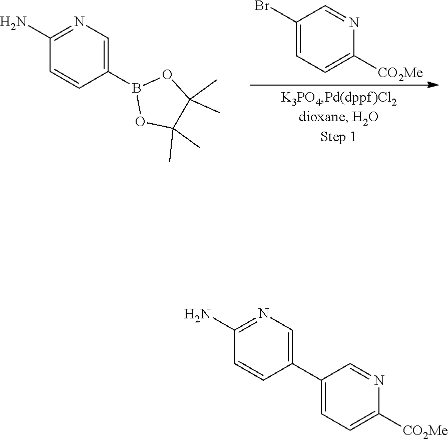 [SMILES: CC1(C)OB(c2ccc(N)nc2)OC1(C)C ; COC(=O)c1ccc(Br)cn1 ; COC(=O)c1ccc(-c2ccc(N)nc2)cn1]

A mixture of 5-(4,4,5,5-tetramethyl-1,3,2-dioxaborolan-2-yl)pyridin-2-amine (300 mg, 1.36 mmol), methyl 5-bromopyridine-2-carboxylate (295 mg, 1.36 mmol), Pd(dppf)Cl₂ (113 mg, 0.139 mmol), and tribasic potassium phosphate tribasic (884 mg, 4.17 mmol) in 1,4-dioxane (5.0 mL) and water (1.0 mL) was heated at 80° C. for 2 hours. Upon completion, the reaction mixture was concentrated under reduced pressure. The crude residue was directly purified by silica gel chromatography, eluted with (petroleum ether/ethyl acetate (20:1) to afford methyl 6′-amino-[3,3′-bipyridine]-6-carboxylate (260 mg, 82%) as a brown solid. LCMS (ESI) [M+H]⁺: 230.2.

### Step 2: Methyl 6′-(1-((4-isopropylphenyl)carbamoyl)-3,3-dimethylazetidine-2-carboxamido)-[3,3′-bipyridine]-6-carboxylate

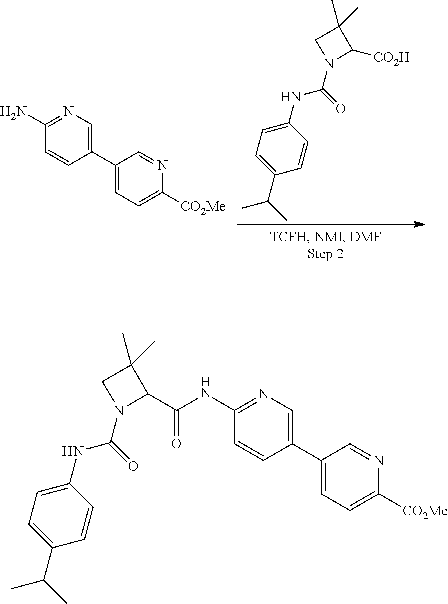 [SMILES: COC(=O)c1ccc(-c2ccc(N)nc2)cn1 ; CC(C)c1ccc(NC(=O)N2CC(C)(C)C2C(=O)O)cc1 ; COC(=O)c1ccc(-c2ccc(NC(=O)C3N(C(=O)Nc4ccc(C(C)C)cc4)CC3(C)C)nc2)cn1]

A solution of 1-[(4-isopropylphenyl)carbamoyl]-3,3-dimethylazetidine-2-carboxylic acid (50 mg, 0.172 mmol), 1-methylimidazole (84.8 mg, 1.03 mmol), methyl 6′-amino-[3,3′-bipyridine]-6-carboxylate (47.4 mg, 0.206 mmol) and TCFH (96.6 mg, 0.344 mmol) in DMF (4.0 mL) was stirred at room temperature for 2 hours. Upon completion, the reaction mixture was concentrated under reduced pressure. The crude residue was directly purified by silica gel column chromatography, eluted with petroleum ether/ethyl acetate (1:1), to afford methyl 6′-(1-((4-isopropylphenyl)carbamoyl)-3,3-dimethylazetidine-2-carboxamido)-[3,3′-bipyridine]-6-carboxylate (75 mg, 86%) as a tan solid. LCMS (ESI) [M+H]⁺: 502.6.

### Step 3: 6′-(1-((4-isopropylphenyl)carbamoyl)-3,3-dimethylazetidine-2-carboxamido)-[3,3′-bipyridine]-6-carboxylic acid

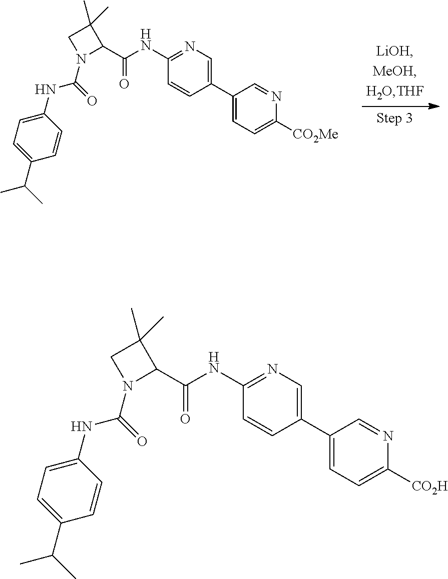 [SMILES: COC(=O)c1ccc(-c2ccc(NC(=O)C3N(C(=O)Nc4ccc(C(C)C)cc4)CC3(C)C)nc2)cn1 ; CC(C)c1ccc(NC(=O)N2CC(C)(C)C2C(=O)Nc2ccc(-c3ccc(C(=O)O)nc3)cn2)cc1]

To methyl 6′-(1-((4-isopropylphenyl)carbamoyl)-3,3-dimethylazetidine-2-carboxamido)-[3,3′-bipyridine]-6-carboxylate (70.0 mg, 0.140 mmol) in methanol (1.0 mL), THF (1.0 mL) and water (1.0 mL) was added lithium hydroxide monohydrate (29.4 mg, 0.70 mmol) at room temperature. After 4 hours, the reaction mixture was concentrated under reduced pressure. The crude residue was directly purified by reversed phase chromatography (conditions: column: C18 silica gel; mobile phase A: water (0.1% formic acid); mobile phase B: MeCN; gradient (B %): 0% to 100% over 20 min; detector: UV 254 nm) to afford 6′-(1-((4-isopropylphenyl)carbamoyl)-3,3-dimethylazetidine-2-carboxamido)-[3,3′-bipyridine]-6-carboxylic acid (45 mg, 66%) as a white solid. LCMS (ESI) [M+H]⁺: 488.6.

### Step 4: (S)-6′-(1-((4-isopropylphenyl)carbamoyl)-3,3-dimethylazetidine-2-carboxamido)-[3,3′-bipyridine]-6-carboxylic acid and (R)-6′-(1-((4-isopropylphenyl)carbamoyl)-3,3-dimethylazetidine-2-carboxamido)-[3,3′-bipyridine]-6-carboxylic acid

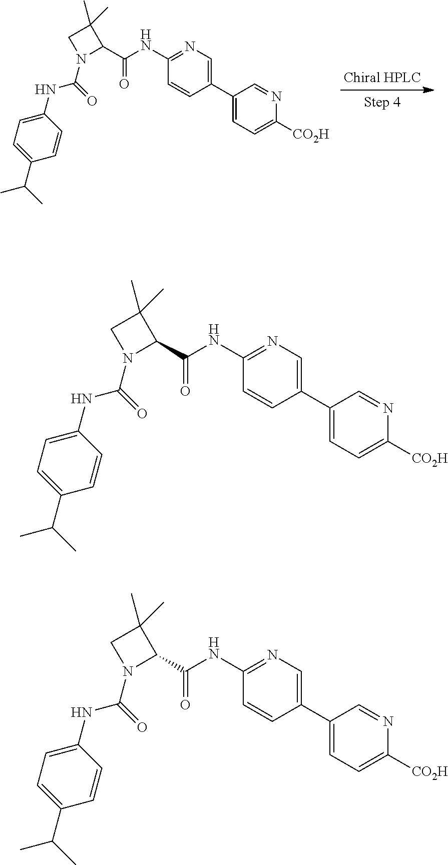 [SMILES: CC(C)c1ccc(NC(=O)N2CC(C)(C)C2C(=O)Nc2ccc(-c3ccc(C(=O)O)nc3)cn2)cc1 ; CC(C)c1ccc(NC(=O)N2CC(C)(C)[C@H]2C(=O)Nc2ccc(-c3ccc(C(=O)O)nc3)cn2)cc1 ; CC(C)c1ccc(NC(=O)N2CC(C)(C)[C@@H]2C(=O)Nc2ccc(-c3ccc(C(=O)O)nc3)cn2)cc1]

6′-(1-((4-isopropylphenyl)carbamoyl)-3,3-dimethylazetidine-2-carboxamido)-[3,3′-bipyridine]-6-carboxylic acid (45 mg) was resolved by preparative chiral HPLC (conditions: Column: CHIRAL ART Cellulose-SZ, 3*25 cm, 5 μm; mobile phase A: MTBE (0.1% formic acid), mobile phase B: methanol; flow rate: 40 mL/min; gradient (B %): isocratic, 50% B; detector: 220/254 nm; RT1 (min): 9; RT2 (min): 21) to afford (Compound 234a) (S)-6′-(1-((4-isopropylphenyl)carbamoyl)-3,3-dimethylazetidine-2-carboxamido)-[3,3′-bipyridine]-6-carboxylic acid (1^{st} peak, 7.7 mg, 18%; Method BL, Peak 1, 0.88 min) as a white solid and (Compound 234b) (R)-6′-(1-((4-isopropylphenyl)carbamoyl)-3,3-dimethylazetidine-2-carboxamido)-[3,3′-bipyridine]-6-carboxylic acid (2^{nd} peak, 7.1 mg, 17%; Method BL, Peak 2, 2.89 min) as a white solid.

| Cmpd # | Characterization Data |
|---|---|
| 234a | LCMS (ESI) [M + H]⁺: 488.2 |
| | ¹H NMR (400 MHz, methanol-d₄) δ 8.97 (s, 1H), 8.70 (s, 1H), 8.43-8.11 (m, 4H), 7.36 (d, J = |
| | 8.1 Hz, 2H), 7.14 (d, J = 8.1 Hz, 2H), 4.55 (s, 1H), 3.91-3.73 (m, 2H), 2.85 (p, J = 6.9 Hz, |
| | 1H), 1.47 (s, 3H), 1.25 (s, 2H), 1.22 (s, 2H), 1.21 (s, 2H). |
| 234b | LCMS (ESI) [M + H]⁺: 488.3 |
| | ¹H NMR (400 MHz, methanol-d₄) δ 9.02-8.89 (m, 1H), 8.74-8.65 (m, 1H), 8.43-8.17 (m, |
| | 4H), 7.36 (d, J = 8.5 Hz, 2H), 7.14 (d, J = 8.4 Hz, 2H), 4.55 (s, 1H), 3.90-3.75 (m, 2H), 2.85 |
| | (p, J = 6.9 Hz, 1H), 1.47 (s, 3H), 1.25 (s, 3H), 1.23 (s, 3H), 1.21 (s, 3H). |

### Example 235: (R)-4-(5-fluoro-6-(4-((4-(1-(trifluoromethyl)cyclopropyl)phenyl)carbamoyl)morpholin-3-yl)pyridin-3-yl)benzoic acid and (S)-4-(5-fluoro-6-(4-((4-(1-(trifluoromethyl)cyclopropyl)phenyl)carbamoyl)morpholin-3-yl)pyridin-3-yl)benzoic acid

### Step 1: 4-(5-fluoro-6-(4-((4-(1-(trifluoromethyl)cyclopropyl)phenyl)carbamoyl)morpholin-3-yl)pyridin-3-yl)benzoic acid

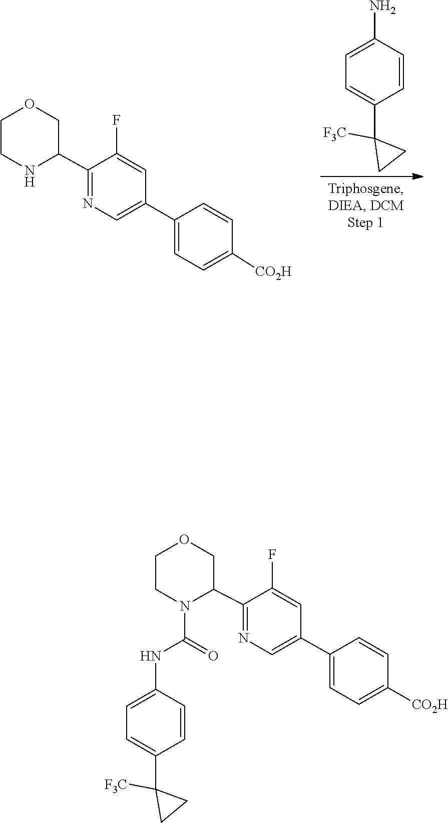 [SMILES: O=C(O)c1ccc(-c2cnc(C3COCCN3)c(F)c2)cc1 ; Nc1ccc(C2(C(F)(F)F)CC2)cc1 ; O=C(O)c1ccc(-c2cnc(C3COCCN3C(=O)Nc3ccc(C4(C(F)(F)F)CC4)cc3)c(F)c2)cc1]

A solution of 4-[1-(trifluoromethyl)cyclopropyl]aniline (34.6 mg, 0.172 mmol), 4-[5-fluoro-6-(morpholin-3-yl)pyridin-3-yl]benzoic acid (40.0 mg, 0.132 mmol), N,N-diisopropylethylamine (85.5 mg, 0.660 mmol) and triphosgene (15.7 mg, 0.053 mmol) in dichloromethane (2.0 mL, 0.070 mmol) was stirred at 25° C. for 1 hour. Upon completion, the reaction mixture was concentrated under reduced pressure. The crude residue was directly purified by reverse phase flash chromatography (conditions: column: C18 silica gel; mobile phase A: water (0.1% formic acid); mobile phase B: MeCN; gradient (B %): 0% to 100% over 25 min; detector: UV 254/220 nm) to give 4-(5-fluoro-6-(4-((4-(1-(trifluoromethyl)cyclopropyl)phenyl)carbamoyl)morpholin-3-yl)pyridin-3-yl)benzoic acid (50 mg, 71%) as an off-white solid. LCMS (ESI) [M+H]⁺: 530.5.

### Step 2: (R)-4-(5-fluoro-6-(4-((4-(1-(trifluoromethyl)cyclopropyl)phenyl)carbamoyl)morpholin-3-yl)pyridin-3-yl)benzoic acid and (S)-4-(5-fluoro-6-(4-((4-(1-(trifluoromethyl)cyclopropyl)phenyl)carbamoyl)morpholin-3-yl)pyridin-3-yl)benzoic acid

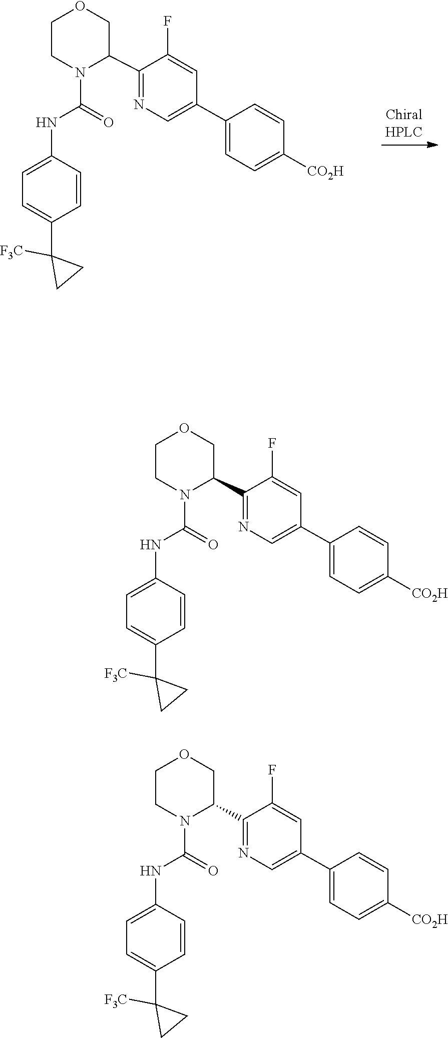 [SMILES: O=C(O)c1ccc(-c2cnc(C3COCCN3C(=O)Nc3ccc(C4(C(F)(F)F)CC4)cc3)c(F)c2)cc1 ; O=C(O)c1ccc(-c2cnc([C@@H]3COCCN3C(=O)Nc3ccc(C4(C(F)(F)F)CC4)cc3)c(F)c2)cc1 ; O=C(O)c1ccc(-c2cnc([C@H]3COCCN3C(=O)Nc3ccc(C4(C(F)(F)F)CC4)cc3)c(F)c2)cc1]

4-(5-fluoro-6-(4-((4-(1-(trifluoromethyl)cyclopropyl)phenyl)carbamoyl)morpholin-3-yl)pyridin-3-yl)benzoic acid (50 mg) was resolved by preparative chiral HPLC (conditions: Column: Lux 5 μm Cellulose-2 2*25 cm, 5 μm; mobile phase A: hexane (0.1% formic acid), mobile phase B: ethanol; flow rate: 20 mL/min; gradient (B %): isocratic, 35% B; detector: 220/254 nm; RT1 (min): 11.5; RT2 (min): 13.4) to afford (Compound 235a) (R)-4-(5-fluoro-6-(4-((4-(1-(trifluoromethyl)cyclopropyl)phenyl)carbamoyl)morpholin-3-yl)pyridin-3-yl)benzoic acid (1^{st} peak, 9.7 mg, 21%; Method BN, Peak 1, 1.09 min) as a white solid and (Compound 235b) (S)-4-(5-fluoro-6-(4-((4-(1-(trifluoromethyl)cyclopropyl)phenyl)carbamoyl)morpholin-3-yl)pyridin-3-yl)benzoic acid (2^{nd} peak, 7.6 mg, 17%; Method BN, Peak 2, 1.30 min) as a white solid.

| Cmpd # | Characterization Data |
|---|---|
| 235a | LCMS (ESI) [M + H]⁺: 530.3 |
| | ¹H NMR (400 MHz, DMSO-d₆) δ 8.74 (d, J = 5.1 Hz, 2H), 8.02-8.35 (m, 3H), 7.84 (d, J = 7.9 |
| | Hz, 2H), 7.44 (d, J = 8.2 Hz, 2H), 7.29 (d, J = 8.3 Hz, 2H), 5.47 (s, 1H), 4.26 (d, J = 11.8 Hz, |
| | 1H), 3.94 (t, J = 10.5 Hz, 5H), 1.25 (s, 2H), 1.03 (s, 2H). |
| 235b | LCMS (ESI) [M + H]⁺: 530.3 |
| | ¹H NMR (400 MHz, DMSO-d₆) δ 8.75 (s, 2H), 8.11 (d, J = 11.9 Hz, 1H), 8.03 (d, J = 8.1 Hz, |
| | 2H), 7.86 (d, J = 7.9 Hz, 2H), 7.44 (d, J = 8.7 Hz, 2H), 7.29 (d, J = 8.4 Hz, 2H), 5.47 (s, 1H), |
| | 4.34-4.08 (m, 2H), 3.93 (s, 4H), 1.25 (s, 2H), 1.03 (s, 2H). |

### Example 236: (R)-5-(4-(5-((3-fluoro-4-isopropylphenyl)carbamoyl)-5-azaspiro[2.3]hexane-4-carboxamido)phenyl)-6-methylpicolinic acid and (S)-5-(4-(5-((3-fluoro-4-isopropylphenyl)carbamoyl)-5-azaspiro[2.3]hexane-4-carboxamido)phenyl)-6-methylpicolinic acid

### Step 1: 5-azaspiro[2.3]hexane-4-carboxylic acid

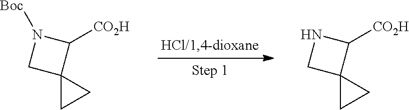 [SMILES: CC(C)(C)OC(=O)N1CC2(CC2)C1C(=O)O ; O=C(O)C1NCC12CC2]

A solution of hydrochloric acid (5.0 mL, 20.0 mmol, 4.0 M in 1,4-dioxane) was added to a solution of 5-(tert-butoxycarbonyl)-5-azaspiro[2.3]hexane-4-carboxylic acid (100 mg, 0.440 mmol) in 1,4-dioxane (2.0 mL) at room temperature. After 1 hour, the reaction mixture was concentrated under reduced pressure to afford crude 5-azaspiro[2.3]hexane-4-carboxylic acid (50 mg, crude) as a yellow oil, which was used directly in the next step without further purification. LCMS (ESI) [M+H]⁺: 128.1.

### Step 2: 5-((3-fluoro-4-isopropylphenyl)carbamoyl)-5-azaspiro[2.3]hexane-4-carboxylic acid

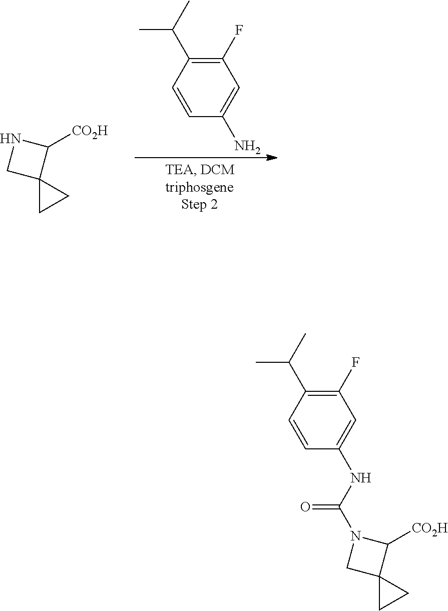 [SMILES: O=C(O)C1NCC12CC2 ; CC(C)c1ccc(N)cc1F ; CC(C)c1ccc(NC(=O)N2CC3(CC3)C2C(=O)O)cc1F]

A solution of 3-fluoro-4-isopropylaniline (90.4 mg, 0.590 mmol), triethylamine (119 mg, 1.18 mmol), triphosgene (46.7 mg, 0.157 mmol), and 5-azaspiro[2.3]hexane-4-carboxylic acid (50 mg, 0.393 mmol) in dichloromethane (5.0 mL) was stirred at room temperature for 1 hour. Upon completion, the reaction mixture was concentrated under reduced pressure. The crude residue was directly purified by silica gel column chromatography, eluted with dichloromethane/methanol (5:1), to afford 5-((3-fluoro-4-isopropylphenyl)carbamoyl)-5-azaspiro[2.3]hexane-4-carboxylic acid (60 mg, 49%) as a yellow oil. LCMS (ESI) [M+H]⁺: 307.1.

### Step 3: Methyl 5-(4-(5-((3-fluoro-4-isopropylphenyl)carbamoyl)-5-azaspiro[2.3]hexane-4-carboxamido)phenyl)-6-methylpicolinate

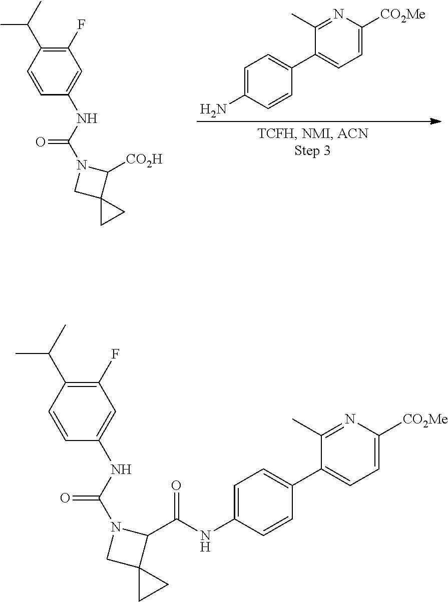 [SMILES: CC(C)c1ccc(NC(=O)N2CC3(CC3)C2C(=O)O)cc1F ; COC(=O)c1ccc(-c2ccc(N)cc2)c(C)n1 ; COC(=O)c1ccc(-c2ccc(NC(=O)C3N(C(=O)Nc4ccc(C(C)C)c(F)c4)CC34CC4)cc2)c(C)n1]

A solution of methyl 5-(4-aminophenyl)-6-methylpyridine-2-carboxylate (71.1 mg, 0.294 mmol), 5-[(3-fluoro-4-isopropylphenyl)carbamoyl]-5-azaspiro[2.3]hexane-4-carboxylic acid (60.0 mg, 0.196 mmol), 1-methylimidazole (80.4 mg, 0.980 mmol), and TCFH (110 mg, 0.392 mmol) in MeCN (3.0 mL) were stirred at room temperature for 1 hour. Upon completion, the reaction mixture was concentrated under reduced pressure. The crude residue was directly purified by silica gel column chromatography, eluted with petroleum ether/ethyl acetate (1:1) to afford methyl 5-(4-(5-((3-fluoro-4-isopropylphenyl)carbamoyl)-5-azaspiro[2.3]hexane-4-carboxamido)phenyl)-6-methylpicolinate (70 mg, 67%) as a yellow oil. LCMS (ESI) [M+H]⁺: 531.2.

### Step 4: 5-(4-(5-((3-fluoro-4-isopropylphenyl)carbamoyl)-5-azaspiro[2.3]hexane-4-carboxamido)phenyl)-6-methylpicolinic acid

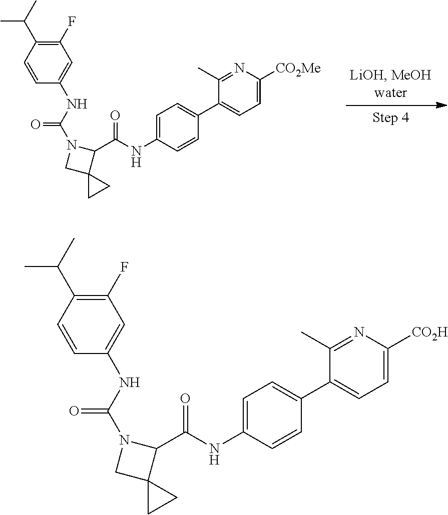 [SMILES: COC(=O)c1ccc(-c2ccc(NC(=O)C3N(C(=O)Nc4ccc(C(C)C)c(F)c4)CC34CC4)cc2)c(C)n1 ; Cc1nc(C(=O)O)ccc1-c1ccc(NC(=O)C2N(C(=O)Nc3ccc(C(C)C)c(F)c3)CC23CC3)cc1]

A solution of methyl 5-(4-(5-((3-fluoro-4-isopropylphenyl)carbamoyl)-5-azaspiro[2.3]hexane-4-carboxamido)phenyl)-6-methylpicolinate (70 mg, 0.132 mmol) and lithium hydroxide monohydrate (27.7 mg, 0.660 mmol) in methanol (1.0 mL) and water (3.0 mL) was stirred at room temperature for 1 hour. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 40 min; detector: UV 254 nm) to afford 5-(4-(5-((3-fluoro-4-isopropylphenyl)carbamoyl)-5-azaspiro[2.3]hexane-4-carboxamido)phenyl)-6-methylpicolinic acid (50 mg, 73%) as a white solid. LCMS (ESI) [M+H]⁺: 517.2.

### Step 5: (R)-5-(4-(5-((3-fluoro-4-isopropylphenyl)carbamoyl)-5-azaspiro[2.3]hexane-4-carboxamido)phenyl)-6-methylpicolinic acid and (S)-5-(4-(5-((3-fluoro-4-isopropylphenyl)carbamoyl)-5-azaspiro[2.3]hexane-4-carboxamido)phenyl)-6-methylpicolinic acid

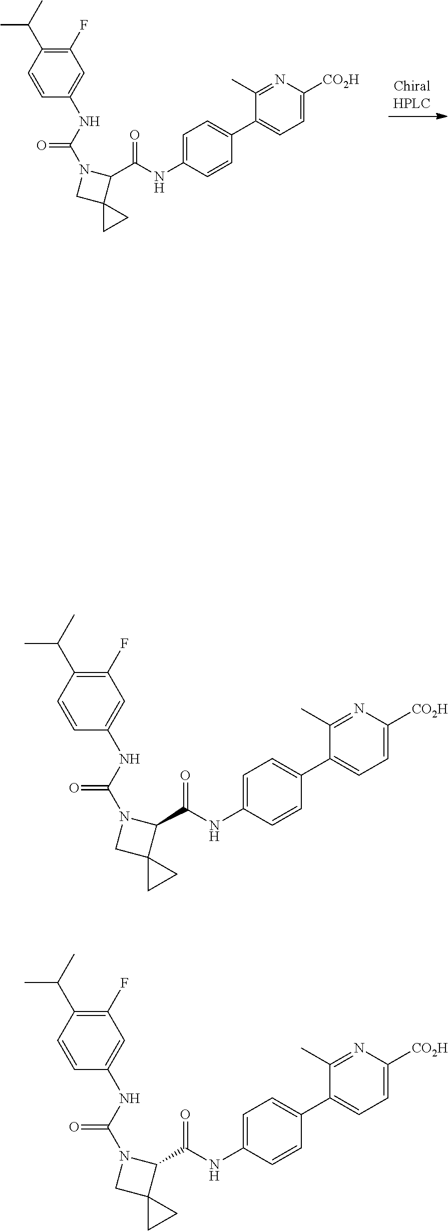 [SMILES: Cc1nc(C(=O)O)ccc1-c1ccc(NC(=O)C2N(C(=O)Nc3ccc(C(C)C)c(F)c3)CC23CC3)cc1 ; Cc1nc(C(=O)O)ccc1-c1ccc(NC(=O)[C@@H]2N(C(=O)Nc3ccc(C(C)C)c(F)c3)CC23CC3)cc1 ; Cc1nc(C(=O)O)ccc1-c1ccc(NC(=O)[C@H]2N(C(=O)Nc3ccc(C(C)C)c(F)c3)CC23CC3)cc1]

5-(4-(5-((3-fluoro-4-isopropylphenyl)carbamoyl)-5-azaspiro[2.3]hexane-4-carboxamido)phenyl)-6-methylpicolinic acid (50 mg) was resolved by preparative chiral HPLC (conditions: column: CHIRALPAK IA, 3*25 cm, 5 μm; mobile phase A: hexane/methyl-tert-butyl ether (1:1; 0.1% formic acid), mobile phase B: methanol; flow rate: 40 mL/min; gradient (B %): isocratic, 20% B; detector: 220/254 nm; RT1 (min): 9.8; RT2 (min): 14.3) to afford (Compound 236a) (R)-5-(4-(5-((3-fluoro-4-isopropylphenyl)carbamoyl)-5-azaspiro[2.3]hexane-4-carboxamido)phenyl)-6-methylpicolinic acid (1^{st} peak, 16.7 mg, 33%; Method BD, Peak 1, 0.95 min) as a white solid and (Compound 236b) (S)-5-(4-(5-((3-fluoro-4-isopropylphenyl)carbamoyl)-5-azaspiro[2.3]hexane-4-carboxamido)phenyl)-6-methylpicolinic acid (2^{nd} peak, 15.7 mg, 31%; Method BD, Peak 2, 1.40 min) as a white solid.

| Cmpd # | Characterization Data |
|---|---|
| 236a | LCMS (ESI) [M + H]⁺: 517.2 |
| | ¹H NMR (400 MHz, DMSO-d₆) δ 9.96 (s, 1H), 8.78 (s, 1H), 7.93 (d, J = 7.8 Hz, 1H), 7.80- |
| | 7.73 (m, 3H), 7.48-7.38 (m, 3H), 7.27-7.14 (m, 2H), 4.74 (s, 1H), 4.17 (d, J = 7.5 Hz, |
| | 1H), 4.07 (d, J = 7.5 Hz, 1H), 3.10-3.05 (m, 1H), 2.49-2.47 (m, 3H), 1.18 (d, J = 6.9 Hz, |
| | 6H), 0.88-0.74 (m, 2H), 0.67-0.50 (m, 2H). |
| 236b | LCMS (ESI) [M + H]⁺: 517.3 |
| | ¹H NMR (400 MHz, DMSO-d₆)) δ 9.96 (s, 1H), 8.78 (s, 1H), 7.93 (d, J = 7.9 Hz, 1H), 7.84- |
| | 7.73 (m, 3H), 7.49-7.38 (m, 3H), 7.25-7.15 (m, 2H), 4.74 (s, 1H), 4.17 (d, J = 7.5 Hz, |
| | 1H), 4.07 (d, J = 7.5 Hz, 1H), 3.08 (p, J = 6.9 Hz, 2H), 2.49-2.45 (m, 3H), 1.18 (d, J = 6.9 |
| | Hz, 6H), 0.89-0.77 (m, 2H), 0.66-0.49 (m, 2H). |

### Example 237: (S)-6-(4-(1-((4-isopropylphenyl)carbamoyl)-3,3-dimethylazetidine-2-carboxamido)phenyl)nicotinic acid and (R)-6-(4-(1-((4-isopropylphenyl)carbamoyl)-3,3-dimethylazetidine-2-carboxamido)phenyl)nicotinic acid

### Step 1: Methyl 6-(4-((tert-butoxycarbonyl)amino)phenyl)nicotinate

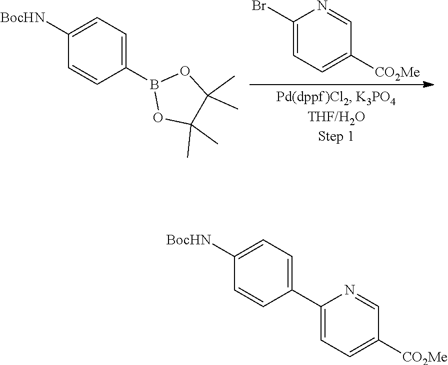 [SMILES: CC(C)(C)OC(=O)Nc1ccc(B2OC(C)(C)C(C)(C)O2)cc1 ; COC(=O)c1ccc(Br)nc1 ; COC(=O)c1ccc(-c2ccc(NC(=O)OC(C)(C)C)cc2)nc1]

A solution of tert-butyl (4-(4,4,5,5-tetramethyl-1,3,2-dioxaborolan-2-yl)phenyl)carbamate (1.0 g, 3.13 mmol), methyl 6-bromopyridine-3-carboxylate (744 mg, 3.44 mmol), Pd(dppf)Cl₂ (256 mg, 0.31 mmol), and tribasic potassium phosphate (1.9 g, 9.39 mmol) in THF (10.0 mL) and water (1.0 mL) was heated at 80° C. for 3 hours. Upon completion, the reaction mixture was concentrated under reduced pressure. The crude residue was directly purified by silica gel column chromatography, eluted with petroleum ether/ethyl acetate (3:1), to afford methyl 6-(4-((tert-butoxycarbonyl)amino)phenyl)nicotinate (400 mg, 38%) as a yellow solid. LCMS (ESI) [M+H]⁺: 329.1.

### Step 2: methyl 6-(4-aminophenyl)nicotinate

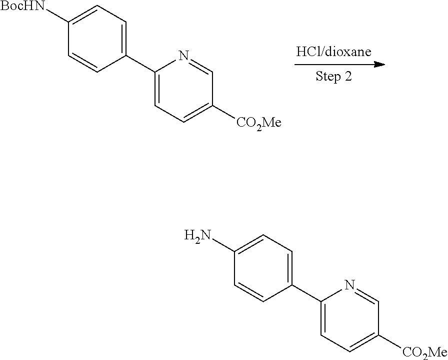 [SMILES: COC(=O)c1ccc(-c2ccc(NC(=O)OC(C)(C)C)cc2)nc1 ; COC(=O)c1ccc(-c2ccc(N)cc2)nc1]

A solution of hydrochloric acid (6.0 mL, 24.0 mmol, 4.0 M in 1,4-dioxane) was added to a solution of methyl 6-(4-((tert-butoxycarbonyl)amino)phenyl)nicotinate (395 mg, 1.20 mmol) in 1,4-dioxane (2 mL) at room temperature. After 3 hours, the reaction mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 40 min; detector: UV 254 nm) to afford methyl 6-(4-aminophenyl)nicotinate (200 mg, 72%) as a pale yellow solid. LCMS (ESI) [M+H]⁺: 229.1.

### Step 3: Methyl 6-(4-(1-((4-isopropylphenyl)carbamoyl)-3,3-dimethylazetidine-2-carboxamido)phenyl)nicotinate

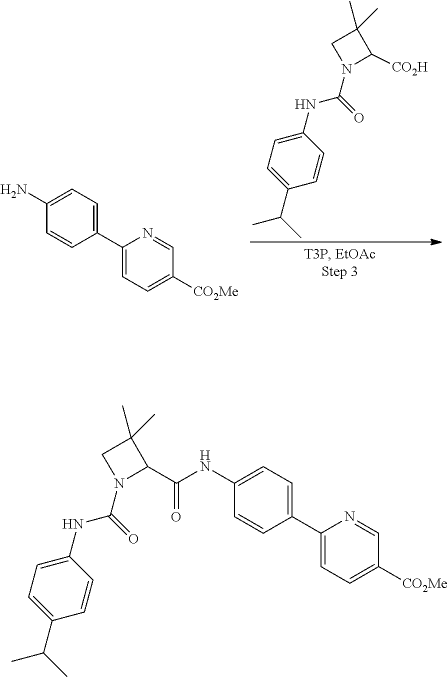 [SMILES: COC(=O)c1ccc(-c2ccc(N)cc2)nc1 ; CC(C)c1ccc(NC(=O)N2CC(C)(C)C2C(=O)O)cc1 ; COC(=O)c1ccc(-c2ccc(NC(=O)C3N(C(=O)Nc4ccc(C(C)C)cc4)CC3(C)C)cc2)nc1]

To a solution of methyl 6-(4-aminophenyl)nicotinate (100.0 mg, 0.42 mmol) and 1-((4-isopropylphenyl)carbamoyl)-3,3-dimethylazetidine-2-carboxylic acid (128 mg, 0.420 mmol) in ethyl acetate (3.0 mL) was added a solution of propane phosphonic acid cyclic anhydride (0.8 mL, 0.86 mmol, 50% in ethyl acetate), and the resultant mixture was stirred at 50° C. for 2 hours. Upon completion, the reaction mixture was concentrated under reduced pressure. The crude residue was directly purified by silica gel column chromatography, eluted with dichloromethane/methanol (10:1), to afford methyl 6-(4-(1-((4-isopropylphenyl)carbamoyl)-3,3-dimethylazetidine-2-carboxamido)phenyl)nicotinate (120 mg, 55%) as an off-white solid. LCMS (ESI) [M+H]⁺: 501.2.

### Step 4: 6-(4-(1-((4-isopropylphenyl)carbamoyl)-3,3-dimethylazetidine-2-carboxamido)phenyl)nicotinic acid

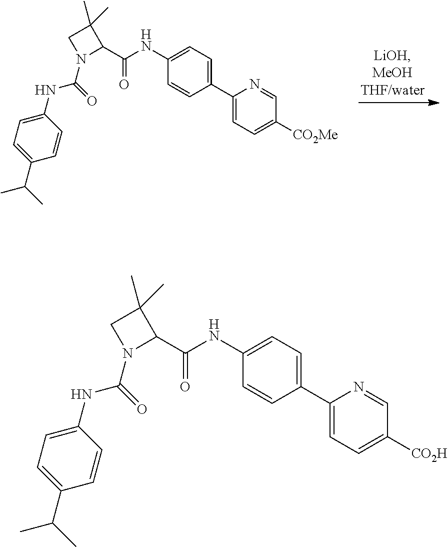 [SMILES: COC(=O)c1ccc(-c2ccc(NC(=O)C3N(C(=O)Nc4ccc(C(C)C)cc4)CC3(C)C)cc2)nc1 ; CC(C)c1ccc(NC(=O)N2CC(C)(C)C2C(=O)Nc2ccc(-c3ccc(C(=O)O)cn3)cc2)cc1]

A solution of methyl 6-(4-(1-((4-isopropylphenyl)carbamoyl)-3,3-dimethylazetidine-2-carboxamido)phenyl)nicotinate (120 mg, 0.240 mmol) and lithium hydroxide monohydrate (126 mg, 3.0 mmol) in methanol (1.0 mL), THF (2.0 mL) and water (3.0 mL) was stirred at room temperature for 1 hour. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 40 min; detector: UV 254 nm) to afford 6-(4-(1-((4-isopropylphenyl)carbamoyl)-3,3-dimethylazetidine-2-carboxamido)phenyl)nicotinic acid (90 mg, 77%) as a white solid. LCMS (ESI) [M+H]⁺: 487.2.

### Step 5: (S)-6-(4-(1-((4-isopropylphenyl)carbamoyl)-3,3-dimethylazetidine-2-carboxamido)phenyl)nicotinic acid and (R)-6-(4-(1-((4-isopropylphenyl)carbamoyl)-3,3-dimethylazetidine-2-carboxamido)phenyl)nicotinic acid

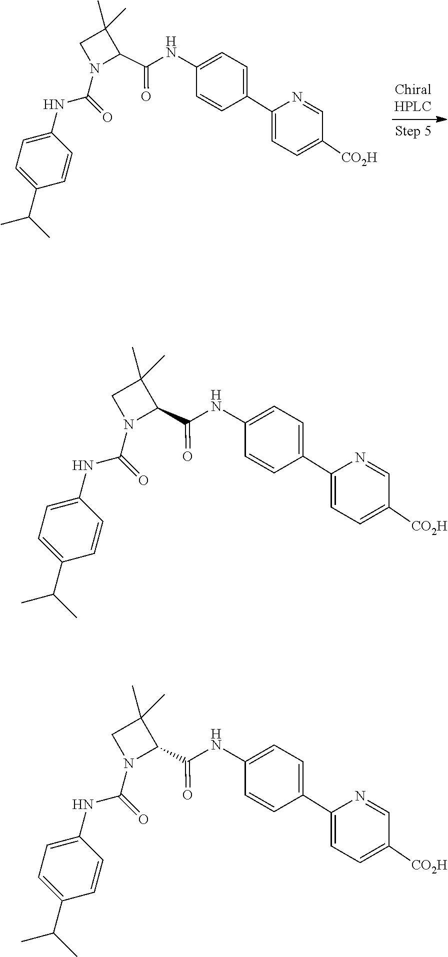 [SMILES: CC(C)c1ccc(NC(=O)N2CC(C)(C)C2C(=O)Nc2ccc(-c3ccc(C(=O)O)cn3)cc2)cc1 ; CC(C)c1ccc(NC(=O)N2CC(C)(C)[C@H]2C(=O)Nc2ccc(-c3ccc(C(=O)O)cn3)cc2)cc1 ; CC(C)c1ccc(NC(=O)N2CC(C)(C)[C@@H]2C(=O)Nc2ccc(-c3ccc(C(=O)O)cn3)cc2)cc1]

6-(4-(1-((4-isopropylphenyl)carbamoyl)-3,3-dimethylazetidine-2-carboxamido)phenyl)nicotinic acid (90 mg) was resolved by preparative chiral HPLC (conditions: Column: CHIRAL ART Cellulose-SZ, 3*25 cm, 5 μm; mobile phase A: MTBE (0.1% formic acid), mobile phase B: methanol; flow rate: 40 mL/min; gradient (B %): isocratic, 20% B; detector: 220/254 nm; RT1 (min): 7; RT2 (min): 11.0) to afford (Compound 237a) (S)-6-(4-(1-((4-isopropylphenyl)carbamoyl)-3,3-dimethylazetidine-2-carboxamido)phenyl)nicotinic acid (1^{st} peak, 36.6 mg, 41%; Method BJ, Peak 1, 0.53 min) as a white solid and (Compound 237b) (R)-6-(4-(1-((4-isopropylphenyl)carbamoyl)-3,3-dimethylazetidine-2-carboxamido)phenyl)nicotinic acid (2^{nd} peak, 32.2 mg, 35%; Method BJ, Peak 2, 0.88 min) as a white solid.

| Cmpd # | Characterization Data |
|---|---|
| 237a | LCMS (ESI) [M + H]⁺: 487.3 |
| | ¹H NMR (400 MHz, methanol-d₄) δ 9.16 (d, J = 2.1 Hz, 1H), 8.39 (dd, J = 8.3, 2.1 Hz, 1H), 8.09- |
| | 8.02 (m, 2H), 7.97 (d, J = 8.4 Hz, 1H), 7.80 (d, J = 8.6 Hz, 2H), 7.35 (d, J = 8.6 Hz, 2H), 7.14 |
| | (d, J = 8.4 Hz, 2H), 4.50 (s, 1H), 3.85 (d, J = 7.3 Hz, 1H), 3.78 (d, J = 7.3 Hz, 1H), 2.85 (p, J = |
| | 7.0 Hz, 1H), 1.47 (s, 3H), 1.26 (s, 3H), 1.23 (s, 3H), 1.21 (s, 3H). |
| 237b | LCMS (ESI) [M + H]⁺: 487.2 |
| | ¹H NMR (400 MHz, methanol-d₄) δ 9.18-9.13 (m, 1H), 8.39 (dd, J = 8.4, 2.2 Hz, 1H), 8.07 (d, J = |
| | 8.8 Hz, 2H), 7.97 (dd, J = 8.3, 0.9 Hz, 1H), 7.80 (d, J = 8.8 Hz, 2H), 7.35 (d, J = 8.6 Hz, 2H), |
| | 7.14 (d, J = 8.5 Hz, 2H), 4.50 (s, 1H), 3.85 (d, J = 7.3 Hz, 1H), 3.78 (d, J = 7.3 Hz, 1H), 2.85 (p, |
| | J = 6.9 Hz, 1H), 1.48 (s, 3H), 1.27 (s, 3H), 1.23 (s, 3H), 1.21 (s, 3H). |

### Example 238: 4-(6-((2S,3S)-4-((2,2-difluorobenzo[d][1,3]dioxol-5-yl)carbamoyl)-2-methylmorpholin-3-yl)-5-fluoropyridin-3-yl)benzoic acid

### Step 1: tert-butyl (2S,3S)-3-(3-fluoro-5-(4-(methoxycarbonyl)phenyl)pyridin-2-yl)-2-methylmorpholine-4-carboxylate

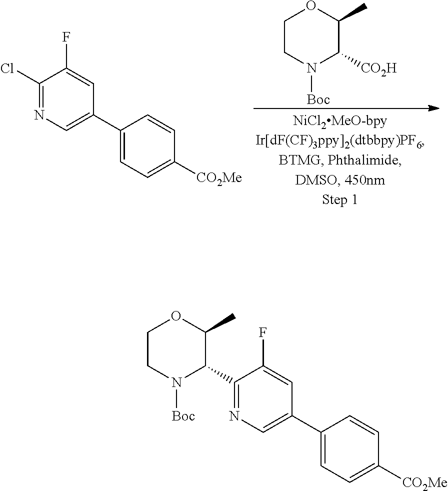 [SMILES: COC(=O)c1ccc(-c2cnc(Cl)c(F)c2)cc1 ; C[C@@H]1OCCN(C(=O)OC(C)(C)C)[C@H]1C(=O)O ; COC(=O)c1ccc(-c2cnc([C@H]3[C@H](C)OCCN3C(=O)OC(C)(C)C)c(F)c2)cc1]

A solution of (2S,3R)-4-(tert-butoxycarbonyl)-2-methylmorpholine-3-carboxylic acid (200 mg, 0.815 mmol), methyl 4-(6-chloro-5-fluoropyridin-3-yl)benzoate (217 mg, 0.815 mmol), NiCl₂·MeO-bpy (17.9 mg, 0.082 mmol), 4,4′-dimethoxy-2,2′-bipyridine (21.2 mg, 0.098 mmol), Ir(dFFppy)2(dtbbpy)PF6 (16.7 mg, 0.016 mmol), N″-tert-butyl-N,N,N′,N′-tetramethylguanidine (209 mg, 1.22 mmol) and 2,3-dihydro-1H-isoindole-1,3-dione (120 mg, 0.815 mmol) in DMSO (6.0 mL) at room temperature was stirred under 450 nm blue LED irradiation for 24 hours. Upon completion, the reaction mixture was poured into water (50 mL) and extracted with ethyl acetate (10×10 mL). The combined organic layers were concentrated under reduced pressure. The crude residue was purified by silica gel column chromatography, eluted with petroleum ether/ethyl acetate (5:1) to afford tert-butyl (2S,3S)-3-(3-fluoro-5-(4-(methoxycarbonyl)phenyl)pyridin-2-yl)-2-methylmorpholine-4-carboxylate (130 mg, 28%) as a yellow oil. LCMS (ESI) [M+H]⁺: 431.1.

### Step 2: Methyl 4-(5-fluoro-6-((2S,3S)-2-methylmorpholin-3-yl)pyridin-3-yl)benzoate

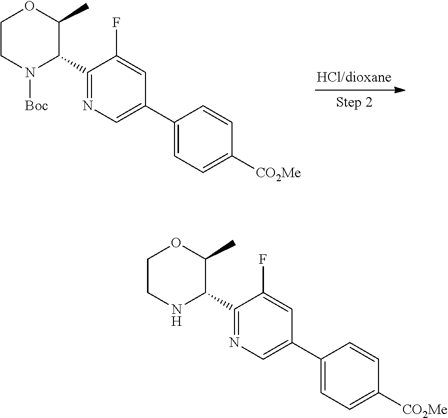 [SMILES: COC(=O)c1ccc(-c2cnc([C@H]3[C@H](C)OCCN3C(=O)OC(C)(C)C)c(F)c2)cc1 ; COC(=O)c1ccc(-c2cnc([C@@H]3NCCO[C@H]3C)c(F)c2)cc1]

To tert-butyl (2S,3S)-3-(3-fluoro-5-(4-(methoxycarbonyl)phenyl)pyridin-2-yl)-2-methylmorpholine-4-carboxylate (130 mg, 0.302 mmol) in 1,4-dioxane (2 mL) at 25° C. was added a solution of hydrochloric acid (2.0 mL, 8.0 mmol, 4.0 M in 1,4-dioxane). After 1 hour, the reaction mixture was concentrated under reduced pressure to afford methyl 4-(5-fluoro-6-((2S,3S)-2-methylmorpholin-3-yl)pyridin-3-yl)benzoate (90 mg, 90%) as a yellow solid, which was used directly in the next step without further purification. LCMS (ESI) [M+H]⁺: 331.1.

### Step 3: 4-(5-fluoro-6-((2S,3S)-2-methylmorpholin-3-yl)pyridin-3-yl)benzoic acid

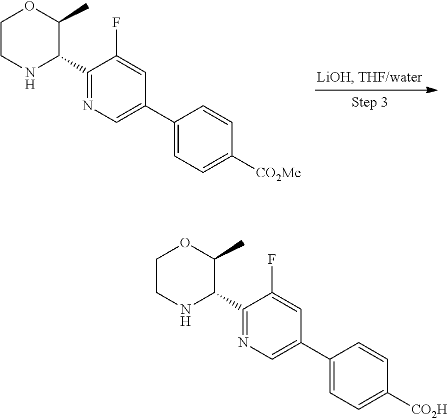 [SMILES: COC(=O)c1ccc(-c2cnc([C@@H]3NCCO[C@H]3C)c(F)c2)cc1 ; C[C@@H]1OCCN[C@H]1c1ncc(-c2ccc(C(=O)O)cc2)cc1F]

A solution of methyl 4-(5-fluoro-6-((2S,3S)-2-methylmorpholin-3-yl)pyridin-3-yl)benzoate (90 mg, 0.272 mmol) and lithium hydroxide monohydrate (57.2 mg, 1.36 mmol) in THF (2.0 mL) and water (0.30 mL) was stirred at 25° C. for 6 hours. Upon completion, the reaction mixture was concentrated under reduced pressure. The crude residue was directly purified by reversed phase chromatography (conditions: column: C18 silica gel; mobile phase A: water (0.1% formic acid); mobile phase B: MeCN; gradient (B %): 0% to 100% over 25 min; detector: UV 254 nm) to afford 4-(5-fluoro-6-((2S,3S)-2-methylmorpholin-3-yl)pyridin-3-yl)benzoic acid (65 mg, 75%) as a white solid. LCMS (ESI) [M+H]⁺: 317.2.

### Step 4: 4-(6-((2S,3S)-4-((2,2-difluorobenzo[d][1,3]dioxol-5-yl)carbamoyl)-2-methylmorpholin-3-yl)-5-fluoropyridin-3-yl)benzoic acid

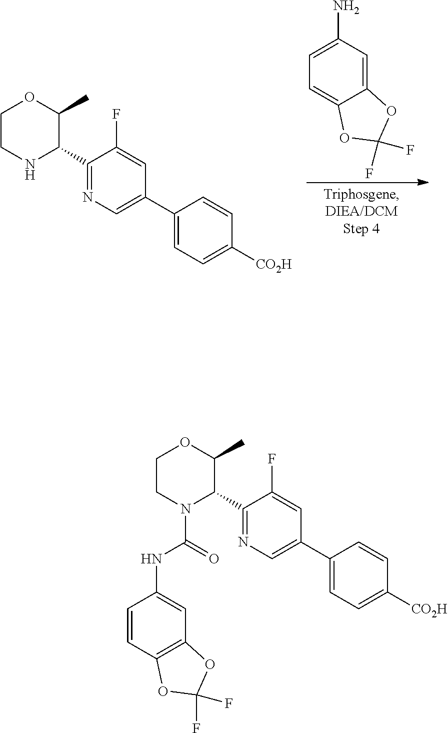 [SMILES: C[C@@H]1OCCN[C@H]1c1ncc(-c2ccc(C(=O)O)cc2)cc1F ; Nc1ccc2c(c1)OC(F)(F)O2 ; C[C@@H]1OCCN(C(=O)Nc2ccc3c(c2)OC(F)(F)O3)[C@H]1c1ncc(-c2ccc(C(=O)O)cc2)cc1F]

A solution of 4-(5-fluoro-6-((2S,3S)-2-methylmorpholin-3-yl)pyridin-3-yl)benzoic acid (65 mg, 0.205 mmol), 2,2-difluoro-1,3-benzodioxol-5-amine (35.6 mg, 0.205 mmol), N,N-Diisopropylethylamine (132 mg, 1.03 mmol) and triphosgene (24.3 mg, 0.082 mmol) in dichloromethane (1.0 mL) was stirred at room temperature for 2 hours. Upon completion, the reaction mixture was concentrated under reduced pressure. The crude residue was directly purified by reverse phase flash chromatography (conditions: column: C18 silica gel; mobile phase A: water (0.1% formic acid); mobile phase B: MeCN; gradient (B %): 0% to 100% over 30 min; detector: UV 254 nm) to afford (Compound 238) 4-(6-((2S,3S)-4-((2,2-difluorobenzo[d][1,3]dioxol-5-yl)carbamoyl)-2-methylmorpholin-3-yl)-5-fluoropyridin-3-yl)benzoic acid (25 mg, 23%) as a white solid.

| Cmpd # | Characterization Data |
|---|---|
| 238 | LCMS (ESI) [M + H]⁺: 516.1 |
| | ¹H NMR (400 MHz, methanol-d₄) δ 8.67 (s, 1H), 8.12 (d, J = 7.8 Hz, 2H), 7.96-7.83 (m, 1H), |
| | 7.78 (d, J = 8.0 Hz, 2H), 7.29 (d, J = 2.0 Hz, 1H), 7.13-6.94 (m, 2H), 4.97 (d, J = 6.2 Hz, 1H), |
| | 4.21 (p, J = 6.3 Hz, 1H), 4.15-3.98 (m, 2H), 3.92-3.81 (m, 1H), 3.72-3.62 (m, 1H), 1.30 (d, J = |
| | 6.6 Hz, 3H). |

### Example 239: 4-(6-((2R,3R)-1-((2,2-difluorobenzo[d][1,3]dioxol-5-yl)carbamoyl)-3-methylpyrrolidin-2-yl)-5-fluoropyridin-3-yl)benzoic acid

### Step 1: tert-butyl (2R,3R)-2-(5-(4-(tert-butoxycarbonyl)phenyl)-3-fluoropyridin-2-yl)-3-methylpyrrolidine-1-carboxylate

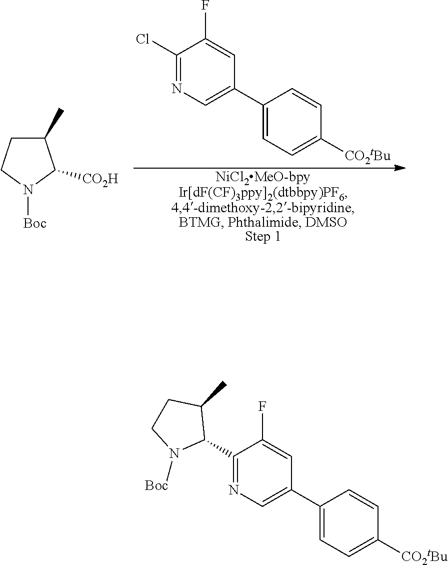

A solution of (2R,3R)-1-(tert-butoxycarbonyl)-3-methylpyrrolidine-2-carboxylic acid (1.00 g, 4.32 mmol), tert-butyl 4-(6-chloro-5-fluoropyridin-3-yl)benzoate (894 mg, 2.90 mmol), dichloronickel;1,2-dimethoxyethane (63.0 mg, 0.291 mmol), 4,4′-dimethoxy-2,2′-bipyridine (75.0 mg, 0.349 mmol), N″-tert-butyl-N,N,N′,N′-tetramethylguanidine (750 mg, 4.36 mmol), Ir[dF(CF3)ppy]2(dtbpy))PF6 (65 mg, 58.0 μmol), and 2,3-dihydro-1H-isoindole-1,3-dione (427 mg, 2.90 mmol) in DMSO (15.0 mL) at room temperature was stirred under 450 nm blue LED irradiation for 24 hours. Upon completion, the reaction mixture was diluted with water and extracted with ethyl acetate (3×30 mL). The combined organic layers were washed with brine, dried over anhydrous Na₂SO₄, filtered and concentrated under reduced pressure. The crude residue was purified by silica gel column chromatography, eluted with petroleum ether/ether (1:1) to afford tert-butyl (2R,3R)-2-(5-(4-(tert-butoxycarbonyl)phenyl)-3-fluoropyridin-2-yl)-3-methylpyrrolidine-1-carboxylate (450 mg, 33%) as a white solid. LCMS (ESI) [M+H]⁺: 457.2.

### Step 2: 4-(5-fluoro-6-((2R,3R)-3-methylpyrrolidin-2-yl)pyridin-3-yl)benzoic acid

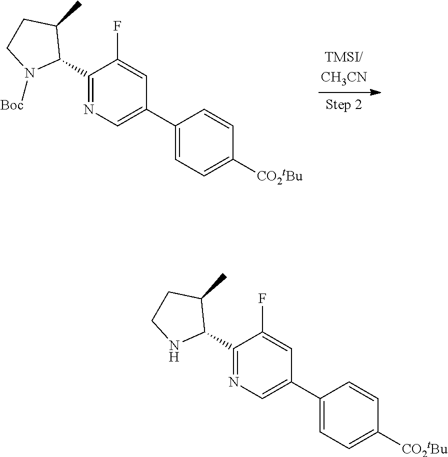 [SMILES: C[C@@H]1CCN(C(=O)OC(C)(C)C)[C@H]1c1ncc(-c2ccc(C(=O)OC(C)(C)C)cc2)cc1F ; C[C@@H]1CCN[C@H]1c1ncc(-c2ccc(C(=O)OC(C)(C)C)cc2)cc1F]

To a solution of tert-butyl (2R,3R)-2-(5-(4-(tert-butoxycarbonyl)phenyl)-3-fluoropyridin-2-yl)-3-methylpyrrolidine-1-carboxylate (300 mg, 0.657 mmol) in MeCN (5.0 mL) at 0° C. was added trimethylsilyl iodide (394 mg, 1.97 mmol). After 5 minutes, the reaction mixture was warmed to room temperature continued to stir for 2 hours. Upon completion, the reaction mixture was concentrated under reduced pressure to afford 4-(5-fluoro-6-((2R,3R)-3-methylpyrrolidin-2-yl)pyridin-3-yl)benzoic acid (280 mg, crude) as a tan solid, which was used directly in the next step without further purification. LCMS (ESI) [M+H]⁺: 301.1.

### Step 3: 4-(6-((2R,3R)-1-((2,2-difluorobenzo[d][1,3]dioxol-5-yl)carbamoyl)-3-methylpyrrolidin-2-yl)-5-fluoropyridin-3-yl)benzoic acid

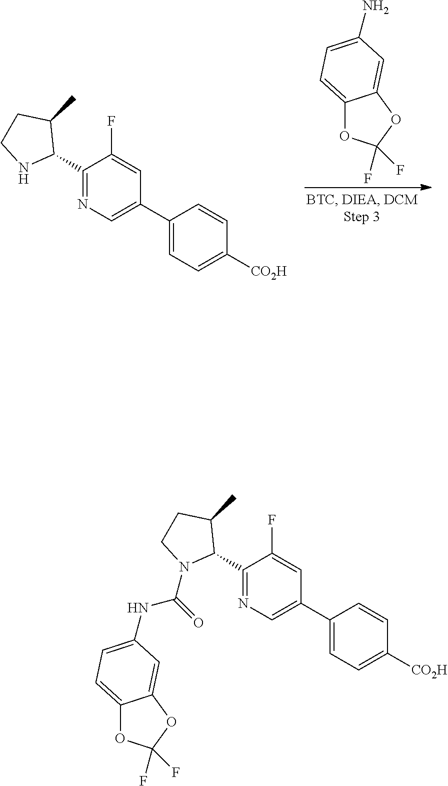 [SMILES: C[C@@H]1CCN[C@H]1c1ncc(-c2ccc(C(=O)O)cc2)cc1F ; Nc1ccc2c(c1)OC(F)(F)O2 ; C[C@@H]1CCN(C(=O)Nc2ccc3c(c2)OC(F)(F)O3)[C@H]1c1ncc(-c2ccc(C(=O)O)cc2)cc1F]

To a mixture of 2,2-difluorobenzo[d][1,3]dioxol-5-amine (155 mg, 0.899 mmol) and N, N-diisopropylethylamine (580 mg, 4.49 mmol) in dichloromethane (10.0 mL) at 0° C. was added triphosgene (106 mg, 0.360 mmol). After 30 minutes, the reaction was warmed to room temperature and 4-(5-fluoro-6-((2R,3R)-3-methylpyrrolidin-2-yl)pyridin-3-yl)benzoic acid (270 mg, 0.899 mmol) was added dropwise over 5 minutes. The reaction was allowed to stir for an additional 2 hours. Upon completion, the reaction mixture was cooled to 0° C. and quenched with ice water. The mixture was extracted with ethyl acetate (3×20 mL). The combined organic layers were dried over anhydrous Na₂SO₄, filtered and concentrated under reduced pressure. The crude residue was purified by reversed-phase flash chromatography (conditions: column: C18 silica gel; mobile phase A: water (0.1% formic acid); mobile phase B: MeCN; gradient (B %): 0% to 100% over 25 min; detector: UV 254 nm) to afford (Compound 239) 4-(6-((2R,3R)-1-((2,2-difluorobenzo[d][1,3]dioxol-5-yl)carbamoyl)-3-methylpyrrolidin-2-yl)-5-fluoropyridin-3-yl)benzoic acid (252 mg, 56%) as a white solid.

| Cmpd # | Characterization Data |
|---|---|
| 239 | LCMS (ESI) [M + H]⁺: 500.2 |
| | ¹H NMR (400 MHz, DMSO-d₆) δ 13.1 (bs, 1H), 8.75 (s, 1H), 8.51 (s, 1H), 8.10-8.02 (m, 3H), |
| | 7.90 (d, J = 8.4 Hz, 2H), 7.56 (s, 1H), 7.22 (d, J = 8.8 Hz, 1H), 7.17-7.14 (m, 1H), 4.84 (d, J = |
| | 6.0 Hz, 1H), 3.75-3.71 (m, 2H), 2.40-2.33 (m, 1H), 2.29-2.9 (m, 1H), 1.75-1.70 (m, 1H), |
| | 1.13 (d, J = 6.8 Hz, 3H). |

### Example 240: (S)-4-(6-(1-((4-isopropylphenyl)carbamoyl)-3,3-dimethylazetidine-2-carboxamido)pyridin-3-yl)-2-methylbenzoic acid and (R)-4-(6-(1-((4-isopropylphenyl)carbamoyl)-3,3-dimethylazetidine-2-carboxamido)pyridin-3-yl)-2-methylbenzoic acid

### Step 1: Methyl 4-(6-aminopyridin-3-yl)-2-methylbenzoate

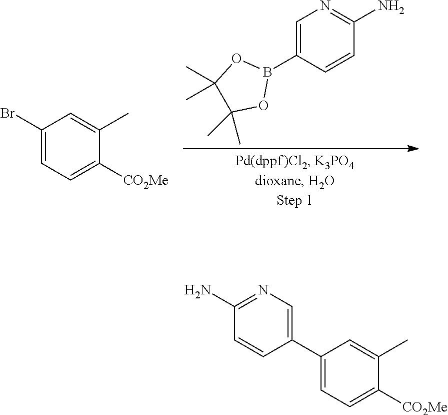 [SMILES: COC(=O)c1ccc(Br)cc1C ; CC1(C)OB(c2ccc(N)nc2)OC1(C)C ; COC(=O)c1ccc(-c2ccc(N)nc2)cc1C]

A solution of methyl 4-bromo-2-methylbenzoate (1.00 g, 4.36 mmol), 5-(4,4,5,5-tetramethyl-1,3,2-dioxaborolan-2-yl)pyridin-2-amine (1.15 g, 5.23 mmol), Pd(dppf)Cl₂ (319 mg, 0.437 mmol) and tribasic potassium phosphate (2.78 g, 13.0 mmol) in 1,4-dioxane (10.0 mL) and water (2.0 mL) was heated at 80° C. for 2 hours. Upon completion, the reaction mixture was extracted with ethyl acetate (3×20 mL). The combined organic layers were dried over anhydrous Na₂SO₄, filtered and concentrated under reduced pressure. The crude residue was purified by reversed-phase flash chromatography (conditions: column: C18 silica gel; mobile phase A: water (0.1% formic acid); mobile phase B: MeCN; gradient (B %): 0% to 100% over 25 min; detector: UV 254 nm) to afford methyl 4-(6-aminopyridin-3-yl)-2-methylbenzoate (900 mg, 85%) as a pale-yellow solid. LCMS (ESI) [M+H]⁺: 243.1.

### Step 2: methyl 4-(6-(1-((4-isopropylphenyl)carbamoyl)-3,3-dimethylazetidine-2-carboxamido)pyridin-3-yl)-2-methylbenzoate

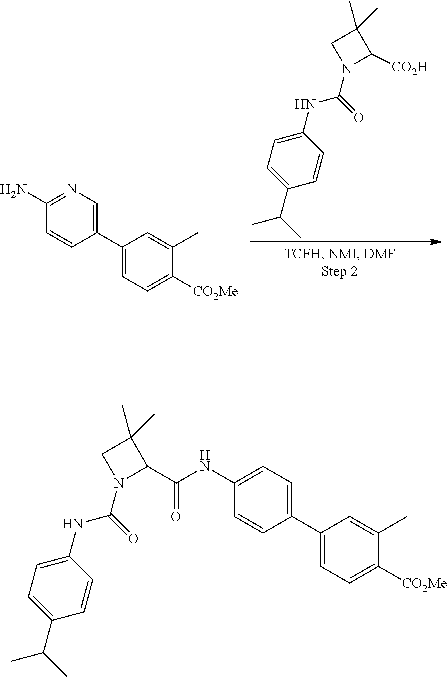 [SMILES: COC(=O)c1ccc(-c2ccc(N)nc2)cc1C ; CC(C)c1ccc(NC(=O)N2CC(C)(C)C2C(=O)O)cc1 ; COC(=O)c1ccc(-c2ccc(NC(=O)C3N(C(=O)Nc4ccc(C(C)C)cc4)CC3(C)C)cc2)cc1C]

To a mixture of 1-((4-isopropylphenyl)carbamoyl)-3,3-dimethylazetidine-2-carboxylic acid (119 mg, 0.413 mmol) in DMF (5.0 mL) at room temperature were added methyl 4-(6-aminopyridin-3-yl)-2-methylbenzoate (100 mg, 0.413 mmol), 1-methyl-1H-imidazole (101 mg, 1.23 mmol) and TCFH (173 mg, 0.619 mmol). After 2 hours, the reaction mixture was concentrated under reduced pressure. The crude residue was directly purified by reversed-phase flash chromatography (conditions: column: C18 silica gel; mobile phase A: water (0.1% formic acid); mobile phase B: MECN; gradient (B %): 0% to 100% over 25 min; detector: UV 254 nm) to afford methyl 4-(6-(1-((4-isopropylphenyl)carbamoyl)-3,3-dimethylazetidine-2-carboxamido)pyridin-3-yl)-2-methylbenzoate (60 mg, 28%) as a white solid. LCMS (ESI) [M+H]⁺: 515.2.

### Step 3: 4-(6-(1-((4-isopropylphenyl)carbamoyl)-3,3-dimethylazetidine-2-carboxamido)pyridin-3-yl)-2-methylbenzoic acid

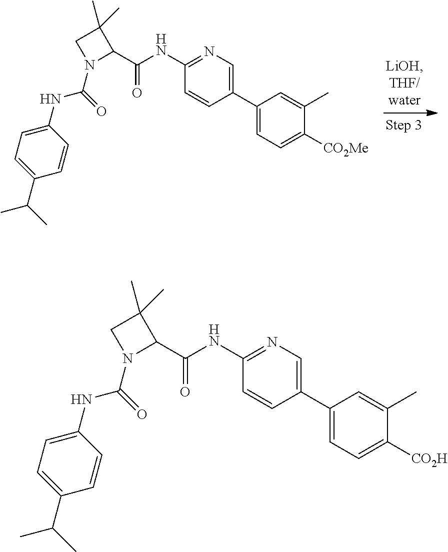 [SMILES: COC(=O)c1ccc(-c2ccc(NC(=O)C3N(C(=O)Nc4ccc(C(C)C)cc4)CC3(C)C)nc2)cc1C ; Cc1cc(-c2ccc(NC(=O)C3N(C(=O)Nc4ccc(C(C)C)cc4)CC3(C)C)nc2)ccc1C(=O)O]

A solution of methyl 4-(6-(1-((4-isopropylphenyl)carbamoyl)-3,3-dimethylazetidine-2-carboxamido)pyridin-3-yl)-2-methylbenzoate (50 mg, 97.0 μmol) and lithium hydroxide monohydrate (12 mg, 0.291 mmol) in THF (1.0 mL) and water (1.0 mL) was stirred at room temperature for 12 hours. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 25 min; detector: UV 254 nm) to afford 4-(6-(1-((4-isopropylphenyl)carbamoyl)-3,3-dimethylazetidine-2-carboxamido)pyridin-3-yl)-2-methylbenzoic acid (28 mg, 57%) as a white solid. LCMS (ESI) [M+H]⁺: 501.2.

### Step 4: (S)-4-(6-(1-((4-isopropylphenyl)carbamoyl)-3,3-dimethylazetidine-2-carboxamido)pyridin-3-yl)-2-methylbenzoic acid and (R)-4-(6-(1-((4-isopropylphenyl)carbamoyl)-3,3-dimethylazetidine-2-carboxamido)pyridin-3-yl)-2-methylbenzoic acid

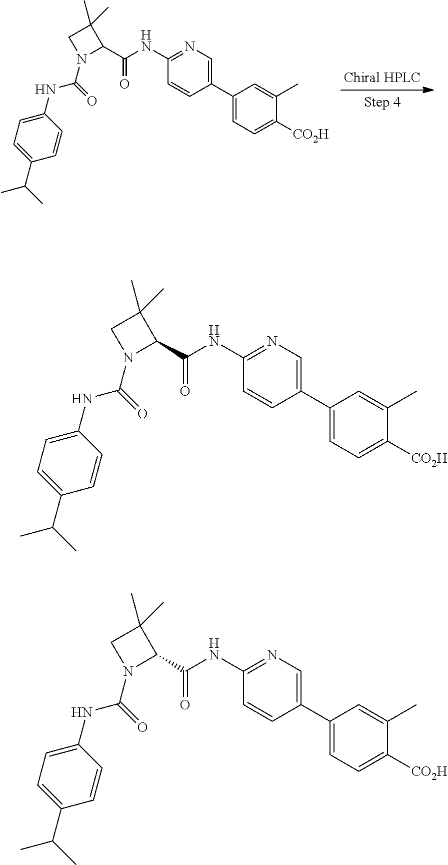 [SMILES: Cc1cc(-c2ccc(NC(=O)C3N(C(=O)Nc4ccc(C(C)C)cc4)CC3(C)C)nc2)ccc1C(=O)O ; Cc1cc(-c2ccc(NC(=O)[C@H]3N(C(=O)Nc4ccc(C(C)C)cc4)CC3(C)C)nc2)ccc1C(=O)O ; Cc1cc(-c2ccc(NC(=O)[C@@H]3N(C(=O)Nc4ccc(C(C)C)cc4)CC3(C)C)nc2)ccc1C(=O)O]

4-(6-(1-((4-isopropylphenyl)carbamoyl)-3,3-dimethylazetidine-2-carboxamido)pyridin-3-yl)-2-methylbenzoic acid (27 mg) was resolved by preparative chiral-HPLC (conditions: Column: CHIRAL ART Cellulose-SZ, 3*25 cm, 5 μm; mobile phase A: MTBE (0.1% formic acid), mobile phase B: methanol; flow rate: 40 mL/min; gradient (B %): isocratic, 50% B; detector: 220/254 nm; RT1 (min): 5.2; RT2 (min): 6.7) to afford (Compound 240a) (S)-4-(6-(1-((4-isopropylphenyl)carbamoyl)-3,3-dimethylazetidine-2-carboxamido)pyridin-3-yl)-2-methylbenzoic acid (1^{st} peak, 9.9 mg, 36%; Method BL, Peak 1, 0.56 min) as a white solid, and (Compound 240b) (R)-4-(6-(1-((4-isopropylphenyl)carbamoyl)-3,3-dimethylazetidine-2-carboxamido)pyridin-3-yl)-2-methylbenzoic acid (2^{nd} peak, 8.4 mg, 31%; Method BL, Peak 2, 0.84 min) as a white solid.

| Cmpd # | Characterization Data |
|---|---|
| 240a | LCMS (ESI) [M + H]⁺: 501.3 |
| | ¹H NMR (400 MHz, DMSO-d₆) δ 12.78 (bs, 1H), 10.53 (s, 1H), 8.73 (s, 1H), 8.47 (s, 1H), |
| | 8.24-8.16 (m, 2H), 7.91 (d, J = 8.0 Hz, 1H), 7.69 (s, 1H), 7.65-7.62 (m, 1H), 7.41 (d, J = |
| | 8.4 Hz, 2H), 7.12 (d, J = 8.4 Hz, 2H), 4.55 (s, 1H), 3.76-3.74 (m, 1H), 3.64-3.62 (m, 1H), |
| | 2.83-2.79 (m, 1H), 2.60 (s, 3H), 1.41 (s, 3H), 1.17 (d, J = 6.8 Hz, 6H), 1.12 (s, 3H). |
| 240b | LCMS (ESI) [M + H]⁺: 501.3 |
| | ¹H NMR (400 MHz, DMSO-d₆) δ 12.8 (bs, 1H), 10.53 (s, 1H), 8.73 (s, 1H), 8.47 (s, 1H), 8.24- |
| | 8.17 (m, 2H), 7.92 (d, J = 8.0 Hz, 1H), 7.69 (s, 1H), 7.65-7.63 (m, 1H), 7.40 (d, J = 8.4 |
| | Hz, 2H), 7.11 (d, J = 8.4 Hz, 2H), 4.55 (s, 1H), 3.76-3.74 (m, 1H), 3.64-3.62 (m, 1H), 2.82- |
| | 2.79 (m, 1H), 2.60 (s, 3H), 1.41 (s, 3H), 1.17 (d, J = 6.8 Hz, 6H), 1.12 (s, 3H). |

### Example 241: (S)—N²-(4-(1H-benzo[d][1,2,3]triazol-5-yl)phenyl)-N¹-(4-isopropylphenyl)-3,3-dimethylazetidine-1,2-dicarboxamide and (R)—N²-(4-(1H-benzo[d][1,2,3]triazol-5-yl)phenyl)-N¹-(4-isopropylphenyl)-3,3-dimethylazetidine-1,2-dicarboxamide

### Step 1: 5-bromo-1-((2-(trimethylsilyl)ethoxy)methyl)-1H-benzo[d][1,2,3]triazole

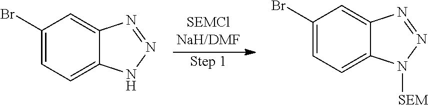

To a solution of 5-bromo-1H-benzo[d][1,2,3]triazole (2.00 g, 10.1 mmol) in DMF (20.0 mL) at 0° C. was added sodium hydride (60% in mineral oil, 363 mg, 15.1 mmol). After 30 minutes, 2-(trimethylsilyl)ethoxymethyl chloride (2.53 g, 15.1 mmol) was added dropwise. The resultant mixture was warmed to room temperature and stirred for 2 hours. Upon completion, the reaction mixture was cooled in an ice bath and quenched with saturated aqueous ammonium chloride (25 mL). The mixture was extracted with ethyl acetate (3×40 mL). The combined organic layers were washed with brine, dried over anhydrous Na₂SO₄, filtered and concentrated under reduced pressure. The crude residue was purified by silica gel column chromatography, eluted with petroleum ether/ethyl acetate (1:1) to afford 5-bromo-1-((2-(trimethylsilyl)ethoxy)methyl)-1H-benzo[d][1,2,3]triazole (2.00 g, 60%) as a pale-yellow solid. LCMS (ESI) [M+H]⁺: 328.0.

### Step 2: 4-(1-((2-(trimethylsilyl)ethoxy)methyl)-1H-benzo[d][1,2,3]triazol-5-yl)aniline

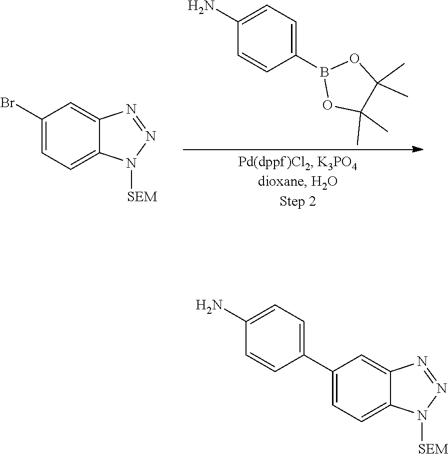 [SMILES: C[Si](C)(C)CCOCn1nnc2cc(Br)ccc21 ; CC1(C)OB(c2ccc(N)cc2)OC1(C)C ; C[Si](C)(C)CCOCn1nnc2cc(-c3ccc(N)cc3)ccc21]

A solution of 5-bromo-1-((2-(trimethylsilyl)ethoxy)methyl)-1H-benzo[d][1,2,3]triazole (2.00 g, 6.09 mmol), 4-(4,4,5,5-tetramethyl-1,3,2-dioxaborolan-2-yl)aniline (1.60 g, 7.31 mmol), Pd(dppf)Cl₂ (445 mg, 0.609 mmol), and tribasic potassium phosphate (3.88 g, 18.2 mmol) in 1,4-dioxane (20.0 mL) and water (4.0 mL) was heated at 80° C. for 2 hours. Upon completion, the reaction mixture was concentrated under reduced pressure. The crude residue was directly purified by silica gel column chromatography, eluted with petroleum ether/ethyl acetate (1:1) to afford 4-(1-((2-(trimethylsilyl)ethoxy)methyl)-1H-benzo[d][1,2,3]triazol-5-yl)aniline (1.80 g, 86%) as a pale-yellow solid. LCMS (ESI) [M+H]⁺: 341.2.

### Step 3: N¹-(4-isopropylphenyl)-3,3-dimethyl-N²-(4-(1-((2-(trimethylsilyl)ethoxy)methyl)-1H-benzo[d][1,2,3]triazol-5-yl)phenyl)azetidine-1,2-dicarboxamide

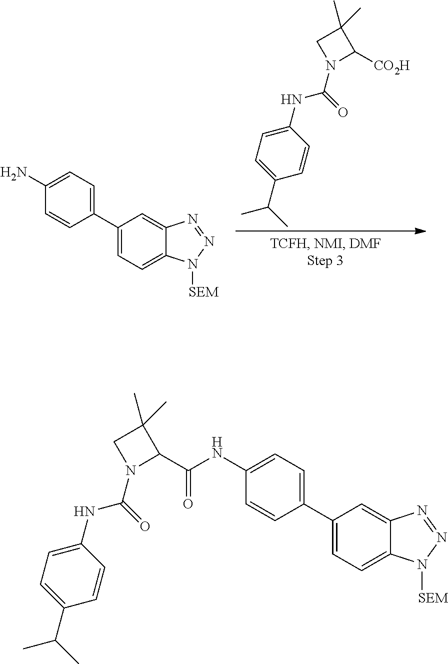

To a mixture of 1-((4-isopropylphenyl)carbamoyl)-3,3-dimethylazetidine-2-carboxylic acid (85.0 mg, 0.294 mmol) in DMF (5.0 mL) at room temperature was added 4-(1-((2-(trimethylsilyl)ethoxy)methyl)-1H-benzo[d][1,2,3]triazol-5-yl)aniline (100 mg, 0.294 mmol), 1-methyl-1H-imidazole (72.0 mg, 0.882 mmol), and TCFH (123 mg, 0.441 mmol). After 2 hours, the reaction mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 25 min; detector: UV 254 nm) to afford N¹-(4-isopropylphenyl)-3,3-dimethyl-N²-(4-(1-((2-(trimethylsilyl)ethoxy)methyl)-1H-benzo[d][1,2,3]triazol-5-yl)phenyl)azetidine-1,2-dicarboxamide (120 mg, 66%) as a pale-yellow solid. LCMS (ESI) [M+H]⁺: 613.3.

### Step 4: N²-(4-(1H-benzo[d][1,2,3]triazol-5-yl)phenyl)-N¹-(4-isopropylphenyl)-3,3-dimethylazetidine-1,2-dicarboxamide

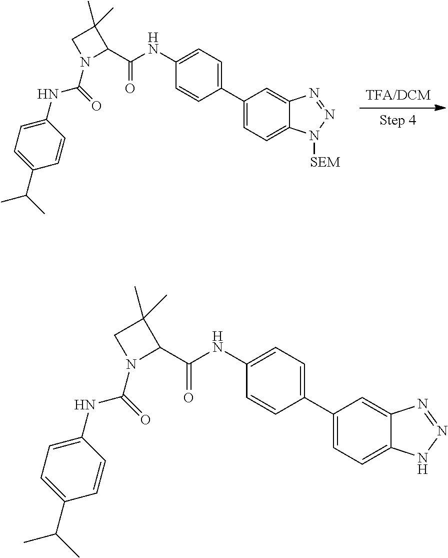 [SMILES: CC(C)c1ccc(NC(=O)N2CC(C)(C)C2C(=O)Nc2ccc(-c3ccc4c(c3)nnn4COCC[Si](C)(C)C)cc2)cc1 ; CC(C)c1ccc(NC(=O)N2CC(C)(C)C2C(=O)Nc2ccc(-c3ccc4[nH]nnc4c3)cc2)cc1]

To N¹-(4-isopropylphenyl)-3,3-dimethyl-N²-(4-(1-((2-(trimethylsilyl)ethoxy)methyl)-1H-benzo[d][1,2,3]triazol-5-yl)phenyl)azetidine-1,2-dicarboxamide (110 mg, 0.179 mmol) in dichloromethane (5.0 mL) at room temperature was added trifluoroacetic acid (1.0 mL). After 12 hours, the reaction mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 25 min; detector: UV 254 nm) to afford N²-(4-(1H-benzo[d][1,2,3]triazol-5-yl)phenyl)-N¹-(4-isopropylphenyl)-3,3-dimethylazetidine-1,2-dicarboxamide (26 mg, 30%) as a white solid. LCMS (ESI) [M+H]⁺: 483.2.

### Step 5: (S)—N²-(4-(1H-benzo[d][1,2,3]triazol-5-yl)phenyl)-N¹-(4-isopropylphenyl)-3,3-dimethylazetidine-1,2-dicarboxamide and (R)—N²-(4-(1H-benzo[d][1,2,3]triazol-5-yl)phenyl)-N¹-(4-isopropylphenyl)-3,3-dimethylazetidine-1,2-dicarboxamide

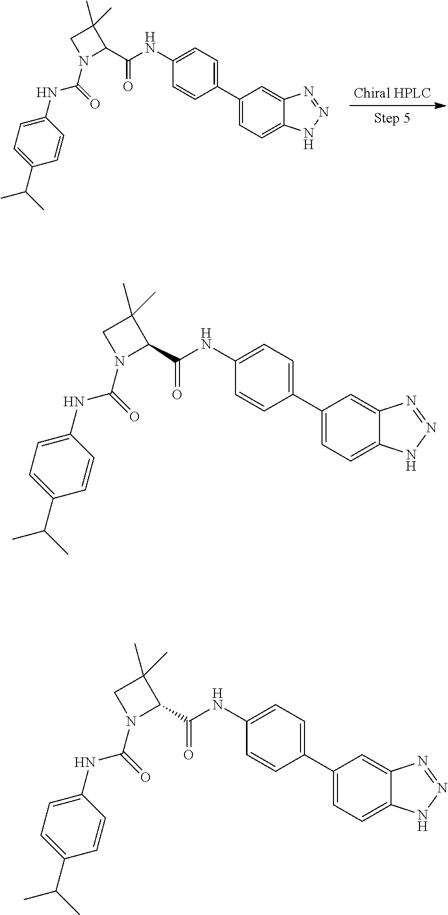 [SMILES: CC(C)c1ccc(NC(=O)N2CC(C)(C)C2C(=O)Nc2ccc(-c3ccc4[nH]nnc4c3)cc2)cc1 ; CC(C)c1ccc(NC(=O)N2CC(C)(C)[C@H]2C(=O)Nc2ccc(-c3ccc4[nH]nnc4c3)cc2)cc1 ; CC(C)c1ccc(NC(=O)N2CC(C)(C)[C@@H]2C(=O)Nc2ccc(-c3ccc4[nH]nnc4c3)cc2)cc1]

N²-(4-(1H-benzo[d][1,2,3]triazol-5-yl)phenyl)-N¹-(4-isopropylphenyl)-3,3-dimethylazetidine-1,2-dicarboxamide (26 mg) was resolved by preparative chiral-HPLC (conditions: column: CHIRALPAK IK 3*25 cm, 5 μm; mobile phase A: hexane (0.5% 2M NH₃-methanol), mobile phase B: ethanol; flow rate: 40 mL/min; gradient (B %): isocratic, 50% B; detector: 220/254 nm; RT1 (min): 5.5; RT2 (min): 11.4) to afford (Compound 241a) (S)—N²-(4-(1H-benzo[d][1,2,3]triazol-5-yl)phenyl)-N^{V}-(4-isopropylphenyl)-3,3-dimethylazetidine-1,2-dicarboxamide (1^{st} peak, 8.8 mg, 35%; Method AK, Peak 1, 0.71 min) as a white solid and (Compound 241b) (R)—N²-(4-(1H-benzo[d][1,2,3]triazol-5-yl)phenyl)-N¹-(4-isopropylphenyl)-3,3-dimethylazetidine-1,2-dicarboxamide (2^{nd} peak, 8.6 mg, 34%; Method AK, Peak 2, 1.40 min) as a white solid.

| Cmpd # | Characterization Data |
|---|---|
| 241a | LCMS (ESI) [M + H]⁺: 483.3 |
| | ¹H NMR (400 MHz, DMSO-d₆) δ 9.97 (s, 1H), 8.46 (s, 1H), 8.08 (s, 1H), 7.98 (d, J = 8.4 Hz, |
| | 1H), 7.82-7.78 (m, 2H), 7.75-7.70 (m, 3H), 7.42 (d, J = 8.4 Hz, 2H), 7.12 (d, J = 8.8 Hz, |
| | 2H), 4.40 (s, 1H), 3.75 (d, J = 7.2 Hz, 1H), 3.67 (d, J = 7.2 Hz, 1H), 2.84-2.78 (m, 1H), 1.40 |
| | (s, 3H), 1.17 (d, J = 6.8 Hz, 6H), 1.13 (s, 3H). |
| 241b | LCMS (ESI) [M + H]⁺: 483.3 |
| | ¹H NMR (400 MHz, DMSO-d₆) δ 15.60 (bs, 1H), 9.97 (s, 1H), 8.46 (s, 1H), 8.08 (s, 1H), 7.98 |
| | (d, J = 8.8 Hz, 1H), 7.80-7.76 (m, 2H), 7.75-7.70 (m, 3H), 7.42 (d, J = 8.8 Hz, 2H), 7.11 (d, J = |
| | 8.4 Hz, 2H), 4.40 (s, 1H), 3.75 (d, J = 7.2 Hz, 1H), 3.66 (d, J = 7.6 Hz, 1H), 2.83-2.79 (m, |
| | 1H), 1.40 (s, 3H), 1.17 (d, J = 6.8 Hz, 6H), 1.13 (s, 3H). |

### Example 242: (S)—N²-(4-(1H-[1,2,3]triazolo[4,5-b]pyridin-5-yl)phenyl)-N¹-(4-isopropylphenyl)-3,3-dimethylazetidine-1,2-dicarboxamide and (R)—N²-(4-(1H-[1,2,3]triazolo[4,5-b]pyridin-5-yl)phenyl)-N¹-(4-isopropylphenyl)-3,3-dimethylazetidine-1,2-dicarboxamide

### Step 1: 5-chloro-1-((2-(trimethylsilyl)ethoxy)methyl)-1H-[1,2,3]triazolo[4,5-b]pyridine

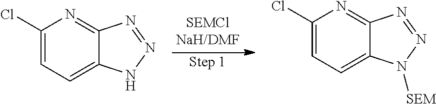

A solution of 5-chloro-1H-[1,2,3]triazolo[4,5-b]pyridine (200 mg, 1.29 mmol) in DMF (6.0 mL) at 0° C. was treated with sodium hydride (60% in mineral oil, 46 mg, 1.94 mmol). After 30 minutes, 2-(trimethylsilyl)ethoxymethyl chloride (323 mg, 1.94 mmol) was added dropwise. The resultant mixture was warmed to room temperature and stirred for 2 hours. Upon completion, the reaction mixture was cooled in an ice bath, diluted with saturated aqueous ammonium chloride (10 mL), and extracted with ethyl acetate (3×20 mL). The combined organic layers were washed with brine, dried over anhydrous Na₂SO₄, filtered and concentrated under reduced pressure. The crude residue was purified by silica gel column chromatography, eluted with petroleum ether/ethyl acetate (1:1), to afford 5-chloro-1-((2-(trimethylsilyl)ethoxy)methyl)-1H-[1,2,3]triazolo[4,5-b]pyridine (300 mg, 81%) as a yellow oil. LCMS (ESI) [M+H]⁺: 285.1.

### Step 2: 4-(1-((2-(trimethylsilyl)ethoxy)methyl)-1H-[1,2,3]triazolo[4,5-b]pyridin-5-yl)aniline

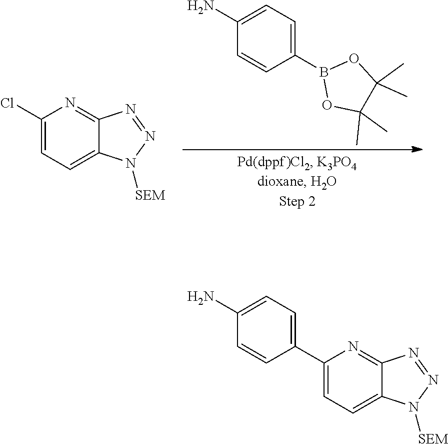 [SMILES: C[Si](C)(C)CCOCn1nnc2nc(Cl)ccc21 ; CC1(C)OB(c2ccc(N)cc2)OC1(C)C ; C[Si](C)(C)CCOCn1nnc2nc(-c3ccc(N)cc3)ccc21]

A solution of 5-chloro-1-((2-(trimethylsilyl)ethoxy)methyl)-1H-[1,2,3]triazolo[4,5-b]pyridine (280 mg, 0.983 mmol), 4-(4,4,5,5-tetramethyl-1,3,2-dioxaborolan-2-yl)aniline (258 mg, 1.18 mmol), Pd(dppf)Cl₂ (71.0 mg, 98.0 μmol) and tribasic potassium phosphate (626 mg, 2.94 mmol) in 1,4-dioxane (5.0 mL) and water (1.0 mL) was heated at 80° C. for 2 hours. Upon completion, the reaction mixture was concentrated under reduced pressure. The crude residue was directly purified by silica gel column chromatography, eluted with petroleum ether/ethyl acetate (1:1), to afford 4-(1-((2-(trimethylsilyl)ethoxy)methyl)-1H-[1,2,3]triazolo[4,5-b]pyridin-5-yl)aniline (310 mg, 92%) as a pale-yellow solid. LCMS (ESI) [M+H]⁺: 342.1.

### Step 3: N¹-(4-isopropylphenyl)-3,3-dimethyl-N²-(4-(1-((2-(trimethylsilyl)ethoxy)methyl)-1H-[1,2,3]triazolo[4,5-b]pyridin-5-yl)phenyl)azetidine-1,2-dicarboxamide

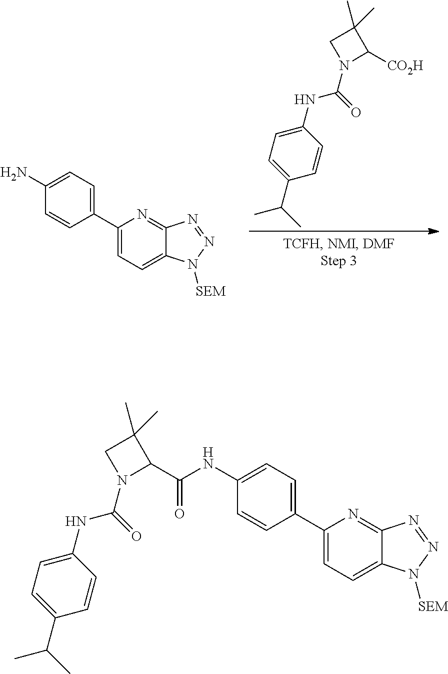 [SMILES: C[Si](C)(C)CCOCn1nnc2nc(-c3ccc(N)cc3)ccc21 ; CC(C)c1ccc(NC(=O)N2CC(C)(C)C2C(=O)O)cc1 ; CC(C)c1ccc(NC(=O)N2CC(C)(C)C2C(=O)Nc2ccc(-c3ccc4c(nnn4COCC[Si](C)(C)C)n3)cc2)cc1]

To a mixture of 1-((4-isopropylphenyl)carbamoyl)-3,3-dimethylazetidine-2-carboxylic acid (85.0 mg, 0.293 mmol) in DMF (6.0 mL) were added 4-(1-((2-(trimethylsilyl)ethoxy)methyl)-1H-[1,2,3]triazolo[4,5-b]pyridin-5-yl)aniline (100 mg, 0.293 mmol), 1-methyl-1H-imidazole (72 mg, 0.879 mmol), and TCFH (123 mg, 0.440 mmol), and the mixture was stirred at room temperature for 6 hours. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 25 min; detector: UV 254 nm) to afford N¹-(4-isopropylphenyl)-3,3-dimethyl-N²-(4-(1-((2-(trimethylsilyl)ethoxy)methyl)-1H-[1,2,3]triazolo[4,5-b]pyridin-5-yl)phenyl)azetidine-1,2-dicarboxamide (100 mg, 55%) as a pale-yellow solid. LCMS (ESI) [M+H]⁺: 614.3.

### Step 4: N²-(4-(1H-[1,2,3]triazolo[4,5-b]pyridin-5-yl)phenyl)-N¹-(4-isopropylphenyl)-3,3-dimethylazetidine-1,2-dicarboxamide

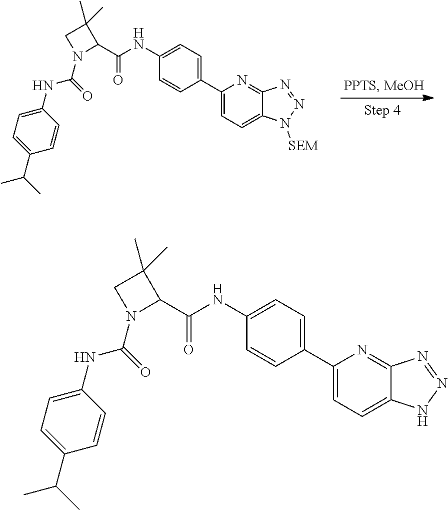 [SMILES: CC(C)c1ccc(NC(=O)N2CC(C)(C)C2C(=O)Nc2ccc(-c3ccc4c(nnn4COCC[Si](C)(C)C)n3)cc2)cc1 ; CC(C)c1ccc(NC(=O)N2CC(C)(C)C2C(=O)Nc2ccc(-c3ccc4[nH]nnc4n3)cc2)cc1]

A solution of N¹-(4-isopropylphenyl)-3,3-dimethyl-N²-(4-(1-((2-(trimethylsilyl)ethoxy)methyl)-1H-[1,2,3]triazolo[4,5-b]pyridin-5-yl)phenyl)azetidine-1,2-dicarboxamide (85 mg, 0.138 mmol) and pyridinium p-toluenesulfonate (104 mg, 0.414 mmol) in methanol (6.0 mL) was heated at 80° C. for 12 hours. Upon completion, the reaction mixture was cooled to room temperature and concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 25 min; detector: UV 254 nm) to afford N²-(4-(1H-[1,2,3]triazolo[4,5-b]pyridin-5-yl)phenyl)-N¹-(4-isopropylphenyl)-3,3-dimethylazetidine-1,2-dicarboxamide (42 mg, 62%) as a white solid. LCMS (ESI) [M+H]⁺: 484.2.

### Step 5: (S)—N²-(4-(1H-[1,2,3]triazolo[4,5-b]pyridin-5-yl)phenyl)-N¹-(4-isopropylphenyl)-3,3-dimethylazetidine-1,2-dicarboxamide and (R)—N²-(4-(1H-[1,2,3]triazolo[4,5-b]pyridin-5-yl)phenyl)-N¹-(4-isopropylphenyl)-3,3-dimethylazetidine-1,2-dicarboxamide

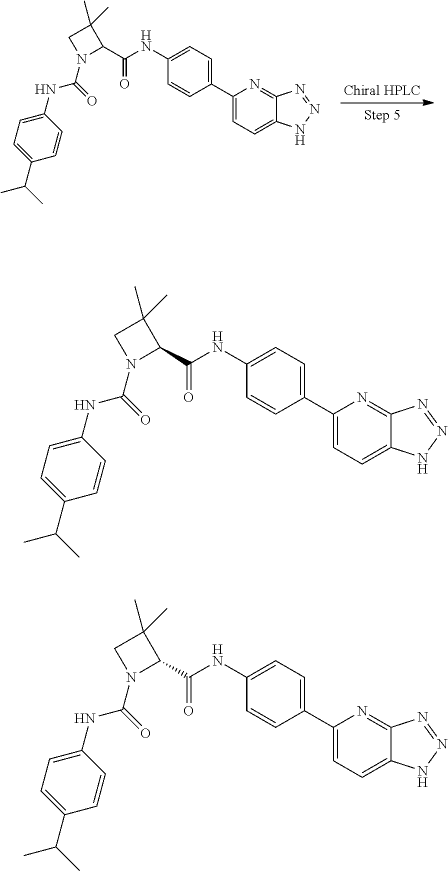 [SMILES: CC(C)c1ccc(NC(=O)N2CC(C)(C)C2C(=O)Nc2ccc(-c3ccc4[nH]nnc4n3)cc2)cc1 ; CC(C)c1ccc(NC(=O)N2CC(C)(C)[C@H]2C(=O)Nc2ccc(-c3ccc4[nH]nnc4n3)cc2)cc1 ; CC(C)c1ccc(NC(=O)N2CC(C)(C)[C@@H]2C(=O)Nc2ccc(-c3ccc4[nH]nnc4n3)cc2)cc1]

N²-(4-(1H-[1,2,3]triazolo[4,5-b]pyridin-5-yl)phenyl)-N¹-(4-isopropylphenyl)-3,3-dimethylazetidine-1,2-dicarboxamide (41 mg) was resolved by preparative chiral-HPLC (conditions: Column: CHIRAL ART Cellulose-SZ, 3*25 cm, 5 μm; mobile phase A: hexane (0.1% formic acid), mobile phase B: ethanol; flow rate: 40 mL/min; gradient (B %): isocratic, 30% B; detector: 220/254 nm; RT1 (min): 14; RT2 (min): 22) to afford (Compound 242a) (S)—N²-(4-(1H-[1,2,3]triazolo[4,5-b]pyridin-5-yl)phenyl)-N¹-(4-isopropylphenyl)-3,3-dimethylazetidine-1,2-dicarboxamide (1^{st} peak, 14.7 mg, 29%; Method BE, Peak 1, 0.88 min) as a white solid and (Compound 242b) (R)—N²-(4-(1H-[1,2,3]triazolo[4,5-b]pyridin-5-yl)phenyl)-N¹-(4-isopropylphenyl)-3,3-dimethylazetidine-1,2-dicarboxamide (2^{nd} peak, 14.1 mg, 28%; Method BE, Peak 2, 1.29 min) as a white solid.

| Cmpd # | Characterization Data |
|---|---|
| 242a | LCMS (ESI) [M + H]⁺: 484.3 |
| | ¹H NMR (400 MHz, DMSO-d₆) δ 10.05 (s, 1H), 8.47-8.45 (m, 2H), 8.17 (d, J = 8.8 Hz, 2H), 8.02 |
| | (d, J = 8.8 Hz, 1H), 7.84 (d, J = 8.8 Hz, 2H), 7.42 (d, J = 8.4 Hz, 2H), 7.11 (d, J = 8.8 Hz, 2H), |
| | 4.42 (s, 1H), 3.75 (d, J = 7.2 Hz, 1H), 3.66 (d, J = 7.2 Hz, 1H), 2.84-2.79 (m, 1H), 1.41 (s, 3H), |
| | 1.17 (d, J = 6.8 Hz, 6H), 1.14 (s, 3H). |
| 242b | LCMS (ESI) [M + H]⁺: 484.3 |
| | ¹H NMR (400 MHz, DMSO-d₆) δ 10.05 (s, 1H), 8.48-8.46 (m, 2H), 8.18 (d, J = 8.4 Hz, 2H), 8.03 |
| | (d, J = 8.8 Hz, 1H), 7.84 (d, J = 8.8 Hz, 2H), 7.42 (d, J = 8.4 Hz, 2H), 7.11 (d, J = 8.4 Hz, 2H), |
| | 4.41 (s, 1H), 3.75 (d, J = 7.2 Hz, 1H), 3.66 (d, J = 7.2 Hz, 1H), 2.84-2.79 (m, 1H), 1.41 (s, 3H), |
| | 1.17 (d, J = 6.8 Hz, 6H), 1.14 (s, 3H). |

### Examples 244-285

Compounds 244-285 in Table 1 were Synthesized Using the Following General Procedures:

### General Synthetic Method 4:

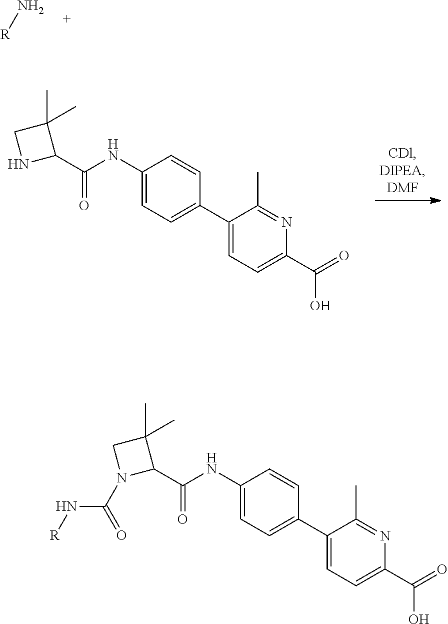

To a reaction vial was added amine building block (1.0 equiv.), followed by a solution of CDI (1.0 equiv.) in anhydrous DMF (1.0 mL) and DIPEA (2.0 equiv.). The mixture was cooled to 0˜5° C. and stirred for 1 hour. Then, 5-(4-(3,3-dimethylazetidine-2-carboxamido)phenyl)-6-methylpicolinic acid (30 mg, 0.088 mmol, 1.0 equiv.) was added to the mixture while stirring. The mixture was slowly warmed to ambient temperature and stirred for 10 hours. The progress of the reaction was analyzed by LCMS. Upon completion, the reaction mixture was filtered with a micropore filter and washed with DMF (1.0 mL). The filtrate was directly purified by prep-HPLC (conditions: Table 3) to afford the desired product.

### General Synthetic Method 5:

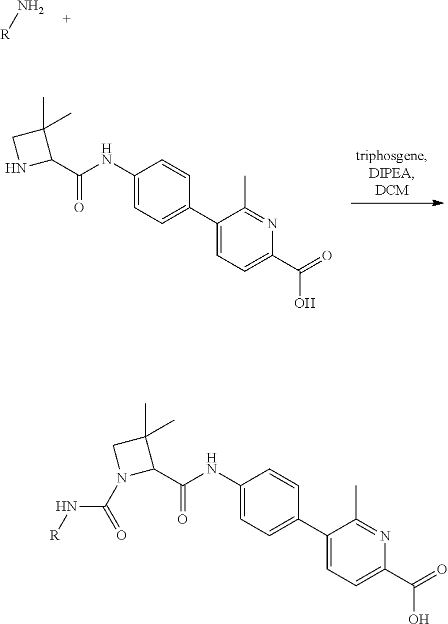

To a reaction vial was added amine building block (1.0 equiv.), DIPEA (1.0 equiv.) and DCM (1.0 mL), followed by a triphosgene (0.33 equiv.). The mixture was cooled to 0˜5° C. and stirred for 20 minutes. Then, a solution of 5-(4-(3,3-dimethylazetidine-2-carboxamido)phenyl)-6-methylpicolinic acid (30 mg, 0.088 mmol, 1.0 equiv.) and DIPEA (3.0 equiv.) in anhydrous DCM (1.0 mL) was added to the mixture while stirring. The reaction mixture was warmed to ambient temperature and stirred for 2 hours. The progress of the reaction was analyzed by LCMS. Upon completion, the reaction mixture was concentrated to dryness under a stream of nitrogen gas. The crude residue was dissolved in DMF (1.0 mL), filtered with a micropore filter and washed with DMF (1.0 mL). The filtrate was directly purified by prep-HPLC (conditions: Table 3) to afford the desired product.

### General Synthetic Method 6:

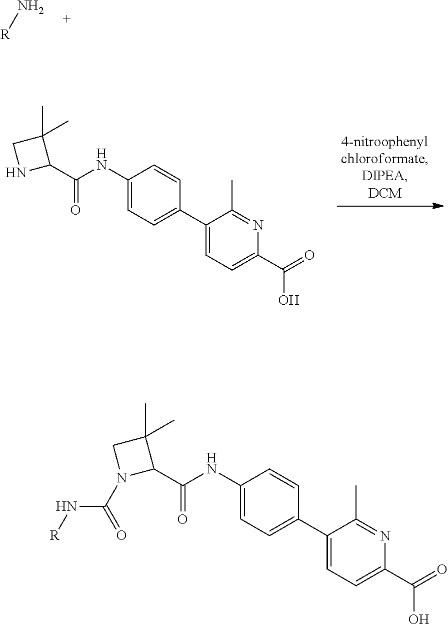

To a reaction vial was added amine building block (1.0 equiv.) and DIPEA (4.0 equiv), followed by a solution of 4-nitrochloroformate (1.1 equiv.) in anhydrous DCM (1.0 mL). The mixture was cooled to 0˜5° C. and stirred for 2 hour. Then, 5-(4-(3,3-dimethylazetidine-2-carboxamido)phenyl)-6-methylpicolinic acid (30 mg, 0.088 mmol, 1.0 equiv.) was added to the mixture while stirring. The mixture was slowly warmed to ambient temperature and stirred for 10 hours. The progress of the reaction was analyzed by LCMS. Upon completion, the reaction mixture was concentrated to dryness under a stream of nitrogen gas. The crude residue was dissolved in DMF (1.0 mL), filtered with a micropore filter and washed with DMF (1.0 mL). The filtrate was directly purified by prep-HPLC (conditions: Table 3) to afford the desired product.

TABLE 3
Synthetic Methods and Characterization Data for Compounds 244-285

| | | Amine Building | | |
|---|---|---|---|---|
| Compd | Method | Block Structure | Prep-HPLC Purification | ¹H NMR; Mass Spectrum m/z |
| # | # | and Name | Conditions | [M + H]⁺ (unless otherwise noted) |
| 244 | 5 | 5- | Column: ASA-AZZOTA- | ¹H NMR (400 MHz, Methanol-d₄) δ |
| | | (trifluoromethoxy)pyridin- | C18-SB-7 μm-30*150 mm, | 8.22 (d, J = 2.9 Hz, 1H), 8.05 (dd, J = |
| | | 2-amine | Mobile phase: A: 0.1% | 8.5, 2.9 Hz, 2H), 7.85 (d, J = 7.9 Hz, |
| | | | FA/H2O B: ACN, Gradient | 1H), 7.77 (d, J = 8.6 Hz, 2H), 7.72- |
| | | | (B %): 34 to 64 in 12 min, | 7.66 (m, 1H), 7.39 (d, J = 8.6 Hz, |
| | | | Flow rate: 35 mL/min, UV | 2H), 4.56 (s, 1H), 3.91 (d, J = 7.4 Hz, |
| | | | wavelength: 220 nm | 1H), 3.83 (d, J = 7.5 Hz, 1H), 2.57 (s, |
| | | | | 3H), 1.50 (s, 3H), 1.27 (s, 3H); 544.2 |
| 245 | 6 | 5-chloro-4- | Column: ASA-AZZOTA- | ¹H NMR (400 MHz, Methanol-d₄) δ |
| | | methylpyridin-2- | C18-SB-7 μm-30*150 mm, | 8.14 (s, 1H), 8.06 (d, J = 7.9 Hz, 1H), |
| | | amine | Mobile phase: A: 0.1% | 7.90 (s, 1H), 7.85 (d, J = 7.9 Hz, 1H), |
| | | | FA/H2O B: ACN, Gradient | 7.81-7.74 (m, 2H), 7.42-7.36 (m, |
| | | | (B %): 36 to 66 in 12 min, | 2H), 4.54 (s, 1H), 3.89 (d, J = 7.4 Hz, |
| | | | Flow rate: 35 mL/min, UV | 1H), 3.82 (d, J = 7.4 Hz, 1H), 2.57 (s, |
| | | | wavelength: 220 nm | 3H), 2.37 (s, 3H), 1.49 (s, 3H), 1.27 |
| | | | | (s, 3H); 508.2 |
| 246 | 4 | (3,3- | Column: Phenomenex-Luna- | ¹H NMR (400 MHz, Methanol-d₄) δ |
| | | dimethylcyclobutyl)methanamine | C18-10 μm-25*250 mm, | 8.06 (d, J = 7.9 Hz, 1H), 7.86 (d, J = |
| | | | Mobile phase: A: 0.1% | 7.9 Hz, 1H), 7.76 (d, J = 8.6 Hz, 2H), |
| | | | FA/H2O B: ACN, Gradient | 7.39 (d, J = 8.6 Hz, 2H), 4.36 (s, 1H), |
| | | | (B %): 34 to 64 in 12 min, | 3.68 (d, J = 7.2 Hz, 1H), 3.60 (d, J = |
| | | | Flow rate: 30 mL/min, UV | 7.2 Hz, 1H), 3.27-3.16 (m, 1H), 3.16- |
| | | | wavelength: 220 nm | 3.04 (m, 1H), 2.58 (s, 3H), 2.49- |
| | | | | 2.35 (m, 1H), 1.88-1.76 (m, 2H), |
| | | | | 1.59-1.47 (m, 2H), 1.41 (s, 3H), |
| | | | | 1.24 (s, 3H), 1.14 (s, 3H), 1.06 (s, |
| | | | | 3H); 479.2 |
| 250 | 5 | 4-(tert- | Column: Phenomenex-Luna- | ¹H NMR (400 MHz, Methanol-d₄) δ |
| | | butyl)aniline | C18-10 μm-25*250 mm, | 8.06 (d, J = 7.9 Hz, 1H), 7.85 (d, J = |
| | | | Mobile phase: A: 0.1% | 7.9 Hz, 1H), 7.77 (d, J = 8.5 Hz, 2H), |
| | | | FA/H2O B: ACN, Gradient | 7.41-7.34 (m, 4H), 7.34-7.28 (m, |
| | | | (B %): 41 to 71 in 12 min, | 2H), 4.50 (s, 1H), 3.89-3.76 (m, |
| | | | Flow rate: 30 mL/min, UV | 2H), 2.57 (s, 3H), 1.47 (s, 3H), 1.28 |
| | | | wavelength: 220 nm | (d, J = 7.2 Hz, 12H); 515.2 |
| 251 | 5 | 2-methyl-4- | Column: YMC-Triart-C18-10 | ¹H NMR (400 MHz, Methanol-d₄) δ |
| | | (trifluoromethyl)aniline | μm-30*250 mm, Mobile | 8.06 (d, J = 7.9 Hz, 1H), 7.86 (d, J = |
| | | | phase: A: 0.1% FA/H2O B: | 7.9 Hz, 1H), 7.81-7.73 (m, 2H), 7.62 |
| | | | ACN, Gradient (B %): 41 to | (d, J = 8.4 Hz, 1H), 7.52-7.48 (m, |
| | | | 71 in 12 min, Flow rate: 35 | 1H), 7.48-7.36 (m, 3H), 4.54 (s, |
| | | | mL/min, UV wavelength″: | 1H), 3.87 (d, J = 7.3 Hz, 1H), 3.81 (d, |
| | | | 220 nm | J = 7.3 Hz, 1H), 2.58 (s, 3H), 2.36 (s, |
| | | | | 3H), 1.51 (s, 3H), 1.28 (s, 3H); 541.2 |
| 252 | 5 | 2,2- | Column: Phenomenex-Luna- | ¹H NMR (400 MHz, Methanol-d₄) δ |
| | | dimethylbenzo[d][1,3]dioxol-5- | C18-10 μm-25*250 mm, | 8.06 (d, J = 7.9 Hz, 1H), 7.85 (d, J = |
| | | amine | Mobile phase: A: 0.1% | 7.9 Hz, 1H), 7.80-7.72 (m, 2H), 7.38 |
| | | | FA/H2O B: ACN, Gradient | (d, J = 8.6 Hz, 2H), 6.95 (d, J = 2.1 |
| | | | (B %): 31 to 61 in 12 min, | Hz, 1H), 6.76 (dd, J = 8.3, 2.1 Hz, |
| | | | Flow rate: 30 mL/min, UV | 1H), 6.62 (d, J = 8.3 Hz, 1H), 4.47 (s, |
| | | | wavelength: 220 nm | 1H), 3.82 (d, J = 7.3 Hz, 1H), 3.76 (d, |
| | | | | J = 7.3 Hz, 1H), 2.57 (s, 3H), 1.62 (s, |
| | | | | 6H), 1.47 (s, 3H), 1.26 (s, 3H); 531.2 |
| 254 | 5 | 4- | Column: Phenomenex-Luna- | ¹H NMR (400 MHz, Methanol-d₄) δ |
| | | (trifluoromethoxy)aniline | C18-10 μm-25*250 mm, | 8.06 (d, J = 7.9 Hz, 1H), 7.85 (d, J = |
| | | | Mobile phase: A: 0.1% | 7.9 Hz, 1H), 7.77 (d, J = 8.6 Hz, 2H), |
| | | | FA/H2O B: ACN, Gradient | 7.56 (d, J = 9.1 Hz, 2H), 7.39 (d, J = |
| | | | (B %): 37 to 67 in 12 min, | 8.5 Hz, 2H), 7.18 (d, J = 8.7 Hz, 2H), |
| | | | Flow rate: 30 mL/min, UV | 4.51 (s, 1H), 3.93-3.73 (m, 2H), |
| | | | wavelength: 220 nm | 2.57 (s, 3H), 1.49 (s, 3H), 1.27 (s, |
| | | | | 3H); 543.2 |
| 255 | 5 | 6-(tert- | Column: Phenomenex-Luna- | ¹H NMR (400 MHz, Methanol-d₄) δ |
| | | butyl)pyridin-3- | C18-10 μm-25*250 mm, | 8.57 (dd, J = 2.6, 0.7 Hz, 1H), 8.05 (d, |
| | | amine | Mobile phase: A: 0.1% | J = 7.9 Hz, 1H), 7.93 (dd, J = 8.7, 2.7 |
| | | | FA/H2O B: ACN, Gradient | Hz, 1H), 7.85 (d, J = 7.8 Hz, 1H), |
| | | | (B %): 13 to 43 in 12 min, | 7.81-7.72 (m, 2H), 7.45-7.35 (m, |
| | | | Flow rate: 30 mL/min, UV | 3H), 4.53 (s, 1H), 3.89 (d, J = 7.4 Hz, |
| | | | wavelength: 220 nm | 1H), 3.82 (d, J = 7.4 Hz, 1H), 2.57 (s, |
| | | | | 3H), 1.49 (s, 3H), 1.35 (d, J = 4.9 Hz, |
| | | | | 9H), 1.27 (s, 3H); 516.2 |
| 256 | 5 | 4- | Column: YMC-Triart-C18-10 | ¹H NMR (400 MHz, Methanol-d₄) δ |
| | | (difluoromethoxy) | μm-30*250 mm, Mobile | 8.06 (d, J = 7.9 Hz, 1H), 7.85 (d, J = |
| | | aniline | phase: A: 0.1% FA/H2O B: | 7.8 Hz, 1H), 7.77 (d, J = 8.2 Hz, 2H), |
| | | | ACN, Gradient (B %): 35 to | 7.53-7.44 (m, 2H), 7.38 (d, J = 8.3 |
| | | | 65 in 12 min, Flow rate: 35 | Hz, 2H), 7.09-6.88 (m, 2H), 6.72 (t, |
| | | | mL/min, UV wavelength: 220 | J = 74.4 Hz, 1H), 4.50 (s, 1H), 3.87 |
| | | | nm | (d, J = 7.3 Hz, 1H), 3.79 (d, J = 7.3 |
| | | | | Hz, 1H), 2.57 (s, 3H), 1.48 (s, 3H), |
| | | | | 1.27 (s, 3H); 525.2 |
| 258 | 5 | 6- | Column: Phenomenex-Luna- | ¹H NMR (400 MHz, DMSO-d₆) δ = |
| | | (trifluoromethoxy)pyridin- | C18-10 μm-25*250 mm, | 10.08 (s, 1H), 9.00 (s, 1H), 8.48 (d, J = |
| | | 3-amine | Mobile phase: A: 0.1% | 2.5 Hz, 1H), 8.16 (dd, J = 2.5, 8.9 |
| | | | FA/H2O B: ACN, Gradient | Hz, 1H), 7.94 (d, J = 7.9 Hz, 1H), |
| | | | (B %): 32 to 62 in 12 min, | 7.83-7.74 (m, 3H), 7.43 (br d, J = 8.5 |
| | | | Flow rate: 30 mL/min, UV | Hz, 2H), 7.25 (d, J = 8.9 Hz, 1H), |
| | | | wavelength: 220 nm | 4.46 (s, 1H), 3.80 (br d, J = 7.4 Hz, |
| | | | | 1H), 3.73 (br d, J = 7.3 Hz, 1H), 2.53- |
| | | | | 2.53 (m, 3H), 1.44 (s, 3H), 1.16 (s, |
| | | | | 3H); 544.2 |
| 259 | 5 | 3-methyl-4- | Column: Phenomenex-Luna- | ¹H NMR (400 MHz, Methanol-d₄) δ |
| | | (trifluoromethyl)aniline | C18-10 μm-25*250 mm, | 8.06 (d, J = 7.9 Hz, 1H), 7.86 (d, J = |
| | | | Mobile phase: A: 0.1% | 7.9 Hz, 1H), 7.83-7.73 (m, 2H), 7.53- |
| | | | FA/H2O B: ACN, Gradient | 7.45 (m, 3H), 7.44-7.33 (m, 2H), |
| | | | (B %): 39 to 69 in 12 min, | 4.53 (s, 1H), 3.96-3.73 (m, 2H), |
| | | | Flow rate: 30 mL/min, UV | 2.58 (s, 3H), 2.42 (d, J = 1.9 Hz, 3H), |
| | | | wavelength: 220 nm | 1.49 (s, 3H), 1.27 (s, 3H); 541.2 |
| 260 | 5 | 3-methoxy-4- | Column: YMC-Triart-C18-10 | ¹H NMR (400 MHz, Methanol-d₄) δ |
| | | (trifluoromethyl)aniline | μm-30*250 mm, Mobile | 8.04 (d, J = 7.7 Hz, 1H), 7.82 (d, J = |
| | | | phase: A: 0.1% FA/H2O B: | 7.7 Hz, 1H), 7.76 (d, J = 8.1 Hz, 2H), |
| | | | ACN, Gradient (B %): 40 to | 7.51 (s, 1H), 7.41 (d, J = 8.6 Hz, 1H), |
| | | | 70 in 12 min, Flow rate: 35 | 7.36 (d, J = 8.1 Hz, 2H), 7.11 (d, J = |
| | | | mL/min, UV wavelength: 220 | 8.3 Hz, 1H), 4.53 (s, 1H), 3.90 (d, J = |
| | | | nm | 7.3 Hz, 1H), 3.86 (s, 3H), 3.82 (d, J = |
| | | | | 7.4 Hz, 1H), 2.54 (s, 3H), 1.49 (s, |
| | | | | 3H), 1.27 (s, 3H); 557.2 |
| 262 | 5 | 4-(tert-butyl)-3- | Column: Phenomenex-Luna- | ¹H NMR (400 MHz, Methanol-d₄) δ |
| | | fluoroaniline | C18-10 μm-25*250 mm, | 8.06 (d, J = 7.9 Hz, 1H), 7.85 (d, J = |
| | | | Mobile phase: A: 0.1% | 7.9 Hz, 1H), 7.81-7.73 (m, 2H), 7.42- |
| | | | FA/H2O B: ACN, Gradient: | 7.35 (m, 2H), 7.31 (dd, J = 15.1, 2.2 |
| | | | 46 to 76 in 12 min, Flow rate: | Hz, 1H), 7.21 (t, J = 8.9 Hz, 1H), 7.13 |
| | | | 30 mL/min, UV wavelength: | (dd, J = 8.6, 2.2 Hz, 1H), 4.50 (s, 1H), |
| | | | 220 nm | 3.86 (d, J = 7.4 Hz, 1H), 3.79 (d, J = |
| | | | | 7.4 Hz, 1H), 2.57 (s, 3H), 1.48 (s, |
| | | | | 3H), 1.34 (d, J = 0.9 Hz, 9H), 1.27 (s, |
| | | | | 3H); 533.2 |
| 263 | 5 | 3-fluoro-4- | Column: YMC-Triart-C18-10 | ¹H NMR (400 MHz, Methanol-d₄) δ |
| | | (trifluoromethyl)aniline | μm-30*250 mm, Mobile | 8.06 (d, J = 7.9 Hz, 1H), 7.85 (d, J = |
| | | | phase: A: 0.1% FA/H2O B: | 7.8 Hz, 1H), 7.80-7.73 (m, 2H), 7.66 |
| | | | ACN, Gradient (B %): 48 to | (dd, J = 13.5, 2.0 Hz, 1H), 7.53 (t, J = |
| | | | 58 in 12 min Flow rate: 35 | 8.5 Hz, 1H), 7.39 (dd, J = 8.7, 2.1 Hz, |
| | | | mL/min, UV wavelength: 220 | 3H), 4.54 (s, 1H), 3.90 (d, J = 7.4 Hz, |
| | | | nm | 1H), 3.82 (d, J = 7.4 Hz, 1H), 2.57 (s, |
| | | | | 3H), 1.50 (s, 3H), 1.27 (s, 3H); 545.2 |
| 264 | 5 | 2-fluoro-4- | Column: Phenomenex-Luna- | ¹H NMR (400 MHz, Methanol-d₄) δ |
| | | (trifluoromethyl)aniline | C18-10 μm-25*250 mm, | 8.09-8.00 (m, 2H), 7.85 (d, J = 7.9 |
| | | | Mobile phase: A: 0.1% | Hz, 1H), 7.82-7.73 (m, 2H), 7.50- |
| | | | FA/H2O B: ACN, Gradient | 7.36 (m, 4H), 4.58 (s, 1H), 3.91 (d, J = |
| | | | (B %): 38 to 68 in 12 min, | 7.4 Hz, 1H), 3.84 (d, J = 7.4 Hz, |
| | | | Flow rate: 30 mL/min, UV | 1H), 2.58 (s, 3H), 1.50 (s, 3H), 1.28 |
| | | | wavelength: 220 nm | (s, 3H); 545.2 |
| 265 | 5 | 2-fluoro-3- | Column: Phenomenex-Luna- | ¹H NMR (400 MHz, DMSO-d₆) δ = |
| | | (trifluoromethoxy)aniline | C18-10 μm-25*250 mm, | 10.09 (s, 1H), 8.61 (s, 1H), 7.93 (d, J = |
| | | | Mobile phase: A: 0.1% | 7.9 Hz, 1H), 7.78 (t, J = 7.5 Hz, |
| | | | FA/H2O B: ACN, Gradient | 3H), 7.74-7.66 (m, 1H), 7.43 (br d, J = |
| | | | (B %): 38 to 68 in 12 min, | 8.5 Hz, 2H), 7.29-7.20 (m, 2H), |
| | | | Flow rate: 30 mL/min, UV | 4.50 (s, 1H), 3.80 (br d, J = 7.4 Hz, |
| | | | wavelength: 220 nm | 1H), 3.70 (br d, J = 7.4 Hz, 1H), 2.53- |
| | | | | 2.52 (m, 3H), 1.45 (s, 3H), 1.15 (s, |
| | | | | 3H); 561.2 |
| 266 | 4 | (S)-1-(4- | Column: ASA-AZZOTA- | ¹H NMR (400 MHz, Methanol-d₄) δ |
| | | (trifluoromethoxy)phenyl)ethan- | C18-7 μm-30*150 mm, | 8.06 (dd, J = 8.0, 3.4 Hz, 1H), 7.86 |
| | | 1-amine | Mobile phase: A: 0.1% | (dd, J = 7.8, 5.1 Hz, 1H), 7.76 (d, J = |
| | | | FA/H2O B: ACN, Gradient | 8.6 Hz, 1H), 7.71 (d, J = 8.5 Hz, 1H), |
| | | | (B %): 38 to 68 in 12 min, | 7.48 (d, J = 8.7 Hz, 1H), 7.44 (d, J = |
| | | | Flow rate: 35 mL/min, UV | 8.6 Hz, 1H), 7.38 (t, J = 8.7 Hz, 2H), |
| | | | wavelength: 220 nm | 7.21 (t, J = 8.8 Hz, 2H), 5.01-4.91 |
| | | | | (m, 1H), 4.39 (d, J = 12.0 Hz, 1H), |
| | | | | 3.75 (dd, J = 7.3, 3.6 Hz, 1H), 3.66 (d, |
| | | | | J = 7.3 Hz, 1H), 2.58 (d, J = 5.9 Hz, |
| | | | | 3H), 1.48 (d, J = 7.1 Hz, 3H), 1.43 (d, |
| | | | | J = 4.5 Hz, 3H), 1.24 (s, 3H); 571.2 |
| 267 | 4 | (2,2- | Column: Phenomenex-Luna- | ¹H NMR (400 MHz, Methanol-d₄) δ |
| | | difluorobenzo[d][1,3]dioxol- | C18-10 μm-25*250 mm, | 8.05 (d, J = 7.9 Hz, 1H), 7.85 (d, J = |
| | | 5-yl)methanamine | Mobile phase: A: 0.1% | 7.9 Hz, 1H), 7.80-7.71 (m, 2H), 7.44- |
| | | | FA/H2O B: ACN, Gradient | 7.35 (m, 2H), 7.22 (s, 1H), 7.12 (d, |
| | | | (B %): 32 to 62 in 12 min, | J = 1.8 Hz, 2H), 4.39 (d, J = 16.2 Hz, |
| | | | Flow rate: 30 mL/min, UV | 2H), 4.32 (d, J = 15.4 Hz, 1H), 3.73 |
| | | | wavelength: 220 nm | (d, J = 7.2 Hz, 1H), 3.65 (d, J = 7.2 |
| | | | | Hz, 1H), 2.57 (s, 3H), 1.45 (s, 3H), |
| | | | | 1.24 (s, 3H); 553.2 |
| 269 | 4 | 3- | Column: Phenomenex-Luna- | ¹H NMR (400 MHz, Methanol-d₄) δ |
| | | phenylcyclobutan- | C18-10 μm-25*250 mm, | 8.06 (d, J = 7.9 Hz, 1H), 7.86 (d, J = |
| | | 1-amine | Mobile phase: A: 0.1% | 7.8 Hz, 1H), 7.80-7.72 (m, 2H), 7.42- |
| | | | FA/H2O B: ACN, Gradient: | 7.35 (m, 2H), 7.32-7.21 (m, 4H), |
| | | | 33 to 63 in12 min, Flow rate: | 7.20-7.10 (m, 1H), 4.38 (d, J = 4.2 |
| | | | 30 mL/min, UV wavelength: | Hz, 1H), 4.23 (tt, J = 9.4, 7.3 Hz, 1H), |
| | | | 220 nm | 3.72 (dd, J = 11.6, 7.2 Hz, 1H), 3.65 |
| | | | | (dd, J = 11.4, 7.3 Hz, 1H), 3.21-3.07 |
| | | | | (m, 1H), 2.77-2.65 (m, 1H), 2.58 (s, |
| | | | | 3H), 2.50 (d, J = 8.0 Hz, 1H), 2.11 |
| | | | | (qd, J = 10.6, 5.4 Hz, 2H), 1.43 (d, J = |
| | | | | 4.3 Hz, 3H), 1.24 (d, J = 4.6 Hz, 3H); |
| | | | | 513.2 |
| 270 | 4 | (S)-2-methoxy-1- | Column: Phenomenex-Luna- | ¹H NMR (400 MHz, Methanol-d₄) δ |
| | | phenylethan-1- | C18-10 μm-25*250 mm, | 8.06 (dd, J = 7.9, 5.5 Hz, 1H), 7.85 |
| | | amine | Mobile phase: A: 0.1% | (dd, J = 9.0, 7.9 Hz, 1H), 7.80-7.74 |
| | | | FA/H2O B: ACN, Gradient | (m, 1H), 7.74-7.68 (m, 1H), 7.44- |
| | | | (B %): 24 to 54 in 12 min, | 7.32 (m, 4H), 7.36-7.28 (m, 2H), |
| | | | Flow rate: 30 mL/min, UV | 7.28-7.20 (m, 1H), 5.05 (ddd, J = |
| | | | wavelength: 220 nm | 13.1, 8.2, 5.2 Hz, 1H), 4.40 (d, J = |
| | | | | 18.4 Hz, 1H), 3.80-3.56 (m, 4H), |
| | | | | 3.38 (d, J = 12.5 Hz, 3H), 2.58 (d, J = |
| | | | | 8.8 Hz, 3H), 1.42 (d, J = 9.1 Hz, 3H), |
| | | | | 1.25 (d, J = 4.1 Hz, 3H); 517.2 |
| 271 | 4 | (4- | Column: YMC-Triart-C18-10 | ¹H NMR (400 MHz, Methanol-d₄) δ |
| | | (trifluoromethoxy)phenyl)methanamine | μm-30*250 mm, Mobile | 8.05 (d, J = 7.9 Hz, 1H), 7.85 (d, J = |
| | | | phase: A: 0.1% FA/H2O B: | 7.8 Hz, 1H), 7.75 (d, J = 8.3 Hz, 2H), |
| | | | ACN, Gradient (B %): 39 to | 7.43 (d, J = 8.5 Hz, 2H), 7.38 (d, J = |
| | | | 69 in 12 min, Flow rate: 35 | 8.2 Hz, 2H), 7.21 (d, J = 8.2 Hz, 2H), |
| | | | mL/min, UV wavelength: 220 | 4.41 (t, J = 7.7 Hz, 2H), 4.34 (d, J = |
| | | | nm | 15.5 Hz, 1H), 3.73 (d, J = 7.3 Hz, |
| | | | | 1H), 3.66 (d, J = 7.3 Hz, 1H), 2.56 (s, |
| | | | | 3H), 1.44 (s, 3H), 1.24 (s, 3H); 557.2 |
| 272 | 4 | (3- | Column: Phenomenex-Luna- | ¹H NMR (400 MHz, Methanol-d₄) δ |
| | | (trifluoromethoxy)phenyl)methanamine | C18-10 μm-25*250 mm, | 8.06 (d, J = 7.9 Hz, 1H), 7.86 (d, J = |
| | | | Mobile phase: A: 0.1% | 7.9 Hz, 1H), 7.76 (d, J = 8.6 Hz, 2H), |
| | | | FA/H2O B: ACN, Gradient | 7.45-7.33 (m, 4H), 7.30-7.11 (m, |
| | | | (B %): 34 to 64 in 12 min, | 2H), 4.50-4.31 (m, 3H), 3.78-3.59 |
| | | | Flow rate: 30 mL/min, UV | (m, 2H), 2.58 (s, 3H), 1.45 (s, 3H), |
| | | | wavelength: 220 nm | 1.24 (s, 3H); 557.2 |
| 273 | 4 | (S)-1-(5-phenyl- | Column: YMC-Triart-C18-10 | ¹H NMR (400 MHz, Methanol-d₄) δ |
| | | 1H-imidazol-2- | μm-30*250 mm″, Mobile | 8.01 (d, J = 8.0 Hz, 1H), 7.88-7.62 |
| | | yl)ethan-1-amine | phase: A: 10 mM | (m, 5H), 7.42-7.18 (m, 6H), 5.08 |
| | | | NH4HCO3/H2O B: ACN, | (dd, J = 7.1, 4.2 Hz, 1H), 4.47 (d, J = |
| | | | Gradient (B %): 17-47, 12, | 12.4 Hz, 1H), 3.86-3.65 (m, 2H), |
| | | | Flow rate: 35 mL/min, UV | 2.54 (d, J = 6.6 Hz, 3H), 1.61 (dd, J = |
| | | | wavelength: 220 nm | 7.1, 4.7 Hz, 3H), 1.47 (d, J = 3.5 Hz, |
| | | | | 3H), 1.25 (d, J = 1.8 Hz, 3H); 553.2 |
| 274 | 5 | 2- | Column: ASA-AZZOTA- | ¹H NMR (400 MHz, Methanol-d₄) δ |
| | | (trifluoromethyl)benzo[d]thiazol- | C18-SB-7 μm-30*150 mm, | 8.40 (d, J = 2.1 Hz, 1H), 8.05 (dd, J = |
| | | 5-amine | Mobile phase: A: 0.1% | 10.0, 8.3 Hz, 2H), 7.85 (d, J = 7.9 Hz, |
| | | | FA/H2O B: ACN, Gradient | 1H), 7.78 (dd, J = 8.8, 2.1 Hz, 3H), |
| | | | (B %): 39 to 69 in 12 min, | 7.42-7.36 (m, 2H), 4.56 (s, 1H), |
| | | | Flow rate: 35 mL/min, UV | 3.93 (d, J = 7.4 Hz, 1H), 3.85 (d, J = |
| | | | wavelength: 220 nm | 7.3 Hz, 1H), 2.58 (s, 3H), 1.51 (s, |
| | | | | 3H), 1.29 (s, 3H); 584.2 |
| 275 | 5 | 2- | Column: Phenomenex-Luna- | ¹H NMR (400 MHz, Methanol-d₄) δ |
| | | (difluoromethoxy) | C18-10 μm-25*250 mm, | 8.05 (d, J = 7.9 Hz, 1H), 7.98 (d, J = |
| | | pyridin-4-amine | Mobile phase: A: 0.1% | 5.8 Hz, 1H), 7.84 (d, J = 7.8 Hz, 1H), |
| | | | FA/H2O B: ACN, Gradient | 7.76 (d, J = 8.2 Hz, 2H), 7.46 (t, J = |
| | | | (B %): 29 to 59 in 12 min, | 73.2 Hz, 1H), 7.38 (d, J = 8.2 Hz, |
| | | | Flow rate: 30 mL/min, UV | 2H), 7.30 (dd, J = 5.9, 1.9 Hz, 1H), |
| | | | wavelength: 220 nm | 7.23 (d, J = 1.8 Hz, 1H), 4.54 (s, 1H), |
| | | | | 3.95-3.76 (m, 2H), 2.56 (s, 3H), |
| | | | | 1.49 (s, 3H), 1.26 (s, 3H); 526.2 |
| 277 | 4 | p- | Column: Phenomenex-Luna- | ¹H NMR (400 MHz, Methanol-d₄) δ |
| | | tolylmethanamine | C18-10 μm-25*250 mm, | 8.05 (d, J = 7.9 Hz, 1H), 7.84 (d, J = |
| | | | Mobile phase: A: 0.1% | 7.8 Hz, 1H), 7.74 (d, J = 8.4 Hz, 2H), |
| | | | FA/H2O B: ACN, Gradient | 7.38 (d, J = 8.3 Hz, 2H), 7.21 (d, J = |
| | | | (B %): 27 to 57 in 12 min, | 7.8 Hz, 2H), 7.11 (d, J = 7.9 Hz, 2H), |
| | | | Flow rate: 30 mL/min, UV | 4.42-4.23 (m, 3H), 3.75-3.58 (m, |
| | | | wavelength: 220 nm | 2H), 2.56 (s, 3H), 2.30 (s, 3H), 1.43 |
| | | | | (s, 3H), 1.24 (s, 3H); 487.2 |
| 278 | 4 | (2-fluoro-4- | Column: YMC-Triart-C18-10 | ¹H NMR (400 MHz, DMSO-d₆) δ = |
| | | (trifluoromethyl)phenyl)methanamine | μm-30*250 mm, Mobile | 10.00 (s, 1H), 7.94 (d, J = 7.9 Hz, |
| | | | phase: A: 0.1% FA/H2O B: | 1H), 7.79 (t, J = 8.3 Hz, 3H), 7.71- |
| | | | ACN, Gradient (B %): 38 to | 7.56 (m, 3H), 7.43 (d, J = 8.6 Hz, |
| | | | 68 in 12 min, Flow rate: 35 | 2H), 7.15 (t, J = 5.9 Hz, 1H), 4.43- |
| | | | mL/min, UV wavelength: 220 | 4.26 (m, 3H), 3.65 (d, J = 7.1 Hz, |
| | | | nm | 1H), 3.57 (d, J = 7.3 Hz, 4H), 2.53 (br |
| | | | | s, 3H), 1.39 (s, 3H), 1.14 (s, 3H); |
| | | | | 559.2 |
| 279 | 4 | 4-(tert- | Column: YMC-Triart-C18-10 | ¹H NMR (400 MHz, Methanol-d₄) δ |
| | | butyl)cyclohexan- | μm-30*250 mm, Mobile | 8.06 (d, J = 7.9 Hz, 1H), 7.85 (dd, J = |
| | | 1-amine | phase: A: 0.1% FA/H2O B: | 7.9, 3.6 Hz, 1H), 7.76 (dd, J = 8.9, 7.1 |
| | | | ACN, Gradient (B %): 50 to | Hz, 2H), 7.44-7.35 (m, 2H), 4.36 (s, |
| | | | 80 in 12 min, Flow rate: 35 | 1H), 3.76-3.65 (m, 1H), 3.65-3.56 |
| | | | mL/min, UV wavelength: 220 | (m, 1H), 3.52-3.40 (m, 1H), 2.58 (d, |
| | | | nm | J = 1.2 Hz, 3H), 1.95 (t, J = 13.6 Hz, |
| | | | | 2H), 1.83 (d, J = 12.7 Hz, 2H), 1.65- |
| | | | | 1.45 (m, 1H), 1.43 (s, 1H), 1.41 (s, |
| | | | | 2H), 1.31-1.12 (m, 4H), 1.09 (dd, J = |
| | | | | 12.7, 3.1 Hz, 1H), 1.04-0.95 (m, |
| | | | | 1H), 0.87 (s, 8H), 0.82 (s, 2H); 521.3 |
| 280 | 4 | (4-(tert- | Column: Phenomenex-Luna- | ¹H NMR (400 MHz, Methanol-d₄) δ |
| | | butyl)phenyl)methanamine | C18-10 μm-25*250 mm, | 8.06 (d, J = 7.9 Hz, 1H), 7.86 (d, J = |
| | | | Mobile phase: A: 0.1% | 7.9 Hz, 1H), 7.79-7.72 (m, 2H), 7.43- |
| | | | FA/H2O B: ACN, Gradient | 7.31 (m, 4H), 7.29-7.22 (m, 2H), |
| | | | (B %): 41 to 71 in 12 min, | 4.39 (s, 1H), 4.38-4.34 (m, 1H), |
| | | | Flow rate: 30 mL/min, UV | 4.28 (d, J = 15.1 Hz, 1H), 3.71 (d, J = |
| | | | wavelength: 220 nm | 7.3 Hz, 1H), 3.65 (d, J = 7.3 Hz, 1H), |
| | | | | 2.58 (s, 3H), 1.43 (s, 3H), 1.29 (s, |
| | | | | 9H), 1.24 (s, 3H); 529.2 |
| 281 | 4 | (4- | Column: YMC-Triart-C18-10 | ¹H NMR (400 MHz, Methanol-d₄) δ |
| | | (trifluoromethyl)phenyl)methanamine | μm-30*250 mm, Mobile | 8.13-8.03 (m, 1H), 7.86 (d, J = 7.8 |
| | | | phase: A: 0.1% FA/H2O B: | Hz, 1H), 7.75 (d, J = 8.4 Hz, 2H), |
| | | | ACN, Gradient (B %): 37 to | 7.61 (d, J = 8.2 Hz, 2H), 7.53 (d, J = |
| | | | 67 in 12 min, Flow rate: 35 | 8.1 Hz, 2H), 7.39 (d, J = 8.3 Hz, 2H), |
| | | | mL/min, UV wavelength: 220 | 4.48 (d, J = 15.7 Hz, 1H), 4.42 (s, |
| | | | nm | 2H), 3.75 (d, J = 7.2 Hz, 1H), 3.67 (d, |
| | | | | J = 7.2 Hz, 1H), 2.58 (s, 3H), 1.45 (s, |
| | | | | 3H), 1.25 (s, 3H); 541.2 |
| 282 | 4 | 4- | Column: YMC-Triart-C18-10 | ¹H NMR (400 MHz, Methanol-d₄) δ |
| | | phenylcyclohexan- | μm-30*250 mm, Mobile | 8.06 (d, J = 7.8 Hz, 1H), 7.89-7.82 |
| | | 1-amine | phase: A: 0.1% FA/H2O B: | (m, 1H), 7.77 (t, J = 8.0 Hz, 2H), 7.39 |
| | | | ACN, Gradient (B %): 44 to | (d, J = 8.3 Hz, 2H), 7.29-7.13 (m, |
| | | | 74 in 12 min, Flow rate: 35 | 4H), 7.17-7.05 (m, 1H), 4.47 (s, |
| | | | mL/min, UV wavelength: 220 | 1H), 3.93 (s, 1H), 3.78-3.58 (m, |
| | | | nm | 2H), 2.58 (d, J = 3.5 Hz, 4H), 1.99 |
| | | | | 1.63 (m, 7H), 1.44 (d, J = 7.2 Hz, |
| | | | | 4H), 1.25 (d, J = 3.2 Hz, 3H); 541.2 |
| 284 | 4 | 1-(4- | Column: YMC-Triart-C18-10 | ¹H NMR (400 MHz, Methanol-d₄) δ |
| | | (trifluoromethyl)phenyl)ethan- | μm-30*250 mm, Mobile | 8.06 (dd, J = 8.0, 4.0 Hz, 1H), 7.85 |
| | | 1-amine | phase: A: 0.1% FA/H2O B: | (dd, J = 7.9, 5.7 Hz, 1H), 7.79-7.72 |
| | | | ACN, Gradient (B %): 40 to | (m, 1H), 7.70 (d, J = 8.5 Hz, 1H), |
| | | | 70 in 12 min, Flow rate: 35 | 7.58 (dq, J = 15.6, 8.5 Hz, 4H), 7.42- |
| | | | mL/min, UV wavelength: 220 | 7.33 (m, 2H), 5.04-4.93 (m, 1H), |
| | | | nm | 4.39 (d, J = 13.4 Hz, 1H), 3.76 (dd, J = |
| | | | | 7.3, 2.0 Hz, 1H), 3.67 (dd, J = 7.3, |
| | | | | 1.7 Hz, 1H), 2.57 (d, J = 6.4 Hz, 3H), |
| | | | | 1.50 (d, J = 7.1 Hz, 3H), 1.44 (d, J = |
| | | | | 4.6 Hz, 3H), 1.24 (s, 3H); 555.2 |
| 285 | 4 | (3-chloro-4- | Column: YMC-Triart-C18-10 | ¹H NMR (400 MHz, DMSO-d₆) δ = |
| | | methylphenyl)methanamine | μm-30*250 mm, Mobile | 9.99 (s, 1H), 7.93 (d, J = 7.9 Hz, 1H), |
| | | | phase: A: 0.1% FA/H2O B: | 7.83-7.74 (m, 3H), 7.41 (d, J = 8.5 |
| | | | ACN, Gradient (B %): 37 to | Hz, 2H), 7.34 (s, 1H), 7.28 (d, J = 7.9 |
| | | | 67 in 12 min, Flow rate: 35 | Hz, 1H), 7.15 (d, J = 8.2 Hz, 1H), |
| | | | mL/min, UV wavelength: 220 | 7.05 (t, J = 6.0 Hz, 1H), 4.29 (s, 1H), |
| | | | nm | 4.19 (br dd, J = 6.0, 11.5 Hz, 2H), |
| | | | | 3.61 (br d, J = 7.1 Hz, 1H), 3.54- |
| | | | | 3.52 (m, 1H), 2.51 (br s, 3H), 2.29 (s, |
| | | | | 3H), 1.36 (s, 3H), 1.12 (s, 3H); 521.2 |

### Example 286: (R)-2-(5-(1H-[1,2,3]triazolo[4,5-b]pyridin-5-yl)-3-fluoropyridin-2-yl)-N-(2,2-difluorobenzo[d][1,3]dioxol-5-yl)pyrrolidine-1-carboxamide

### Step 1: (R)-5-chloro-3-fluoro-2-(pyrrolidin-2-yl)pyridine hydrochloride

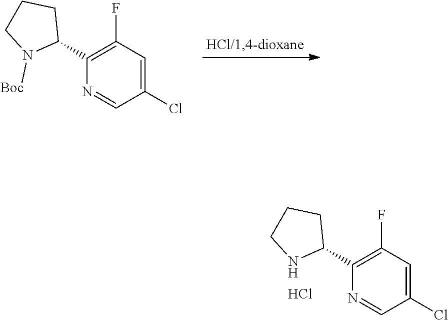 [SMILES: CC(C)(C)OC(=O)N1CCC[C@@H]1c1ncc(Cl)cc1F ; Fc1cc(Cl)cnc1[C@H]1CCCN1 ; Cl]

To a mixture of tert-butyl (R)-2-(5-chloro-3-fluoropyridin-2-yl)pyrrolidine-1-carboxylate (250 mg, 0.830 mmol) in 1,4-dioxane (1 mL) was added in HCl (1 mL, 4.00 mmol, 4 M in 1,4-dioxane) at room temperature. After 1 hour, the resulting mixture was concentrated under reduced pressure to afford (R)-5-chloro-3-fluoro-2-(pyrrolidin-2-yl)pyridine hydrochloride (188 mg, crude) as a yellow solid, which was used directly in the next step without further purification. LCMS (ESI) [M+H]⁺: 201.0.

### Step 2: (R)-2-(5-chloro-3-fluoropyridin-2-yl)-N-(2,2-difluorobenzo[d][1,3]dioxol-5-yl)pyrrolidine-1-carboxamide

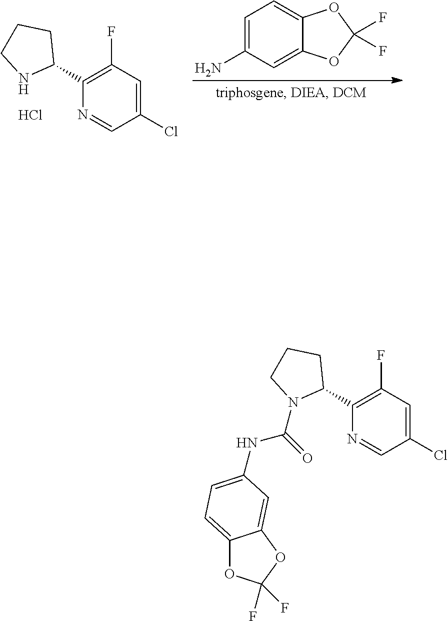 [SMILES: Fc1cc(Cl)cnc1[C@H]1CCCN1 ; Cl ; Nc1ccc2c(c1)OC(F)(F)O2 ; O=C(Nc1ccc2c(c1)OC(F)(F)O2)N1CCC[C@@H]1c1ncc(Cl)cc1F]

To a mixture of 2,2-difluorobenzo[d][1,3]dioxol-5-amine (186 mg, 1.07 mmol) in DCM (6 mL) were added DIEA (1.11 g, 8.58 mmol) and triphosgene (105 mg, 0.350 mmol) at 0° C. under nitrogen atmosphere. After 30 minutes, the reaction was warmed to room temperature and (R)-5-chloro-3-fluoro-2-(pyrrolidin-2-yl)pyridine hydrochloride (178 mg, 0.750 mmol) was added dropwise over 3 minutes. The resulting mixture was stirred at room temperature for an additional 2 hours. Upon completion, the reaction was quenched with ice water at 0° C. The resulting mixture was extracted with EtOAc (3×10 mL). The combined organic layers were dried over anhydrous Na₂SO₄, filtered and concentrated under reduced pressure. The crude residue was purified by silica gel column chromatography, eluted with PE/EA (1:1) to afford (R)-2-(5-chloro-3-fluoropyridin-2-yl)-N-(2,2-difluorobenzo[d][1,3]dioxol-5-yl)pyrrolidine-1-carboxamide (110 mg, 26% yield) as a yellow oil. LCMS (ESI) [M+H]⁺: 400.0.

### Step 3: (R)-(6-(1-((2,2-difluorobenzo[d][1,3]dioxol-5-yl)carbamoyl)pyrrolidin-2-yl)-5-fluoropyridin-3-yl)boronic acid

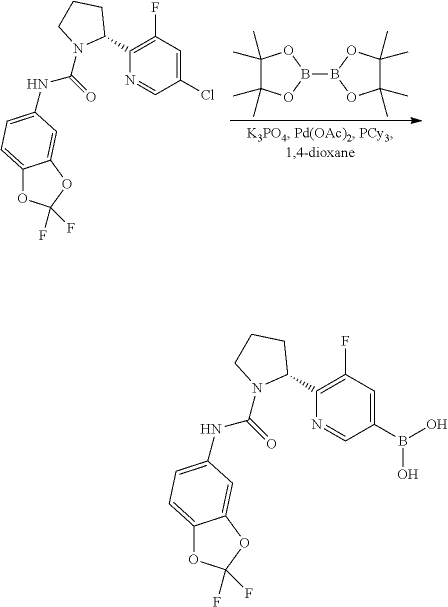

A solution of (R)-2-(5-chloro-3-fluoropyridin-2-yl)-N-(2,2-difluorobenzo[d][1,3]dioxol-5-yl)pyrrolidine-1-carboxamide (100 mg, 0.25 mmol), 4,4,4′,4′,5,5,5′,5′-octamethyl-2,2′-bi(1,3,2-dioxaborolane) (191 mg, 0.75 mmol), K₃PO₄ (159 mg, 0.75 mmol), Pd(OAc)₂ (6 mg, 20 μmol) and PCy₃ (14 mg, 50 μmol) in 1,4-dioxane (5 mL) was stirred at 80° C. overnight under nitrogen atmosphere. Upon completion, the reaction mixture was allowed to cool down to room temperature. The resulting mixture was concentrated under reduced pressure. The crude residue was directly purified by reversed-phase flash chromatography (conditions: column: C18 silica gel; mobile phase A: water (0.1% formic acid), mobile phase B: MeCN; Gradient (B %): 0% to 100% in 25 min; detector, UV 254 nm) to afford (R)-(6-(1-((2,2-difluorobenzo[d][1,3]dioxol-5-yl)carbamoyl)pyrrolidin-2-yl)-5-fluoropyridin-3-yl)boronic acid (50 mg, 49% yield) as a white solid. LCMS (ESI) [M+H]⁺: 410.1.

### Step 4: (R)—N-(2,2-difluorobenzo[d][1,3]dioxol-5-yl)-2-(3-fluoro-5-(1-((2-(trimethylsilyl)ethoxy)methyl)-1H-[1,2,3]triazolo[4,5-b]pyridin-5-yl)pyridin-2-yl)pyrrolidine-1-carboxamide

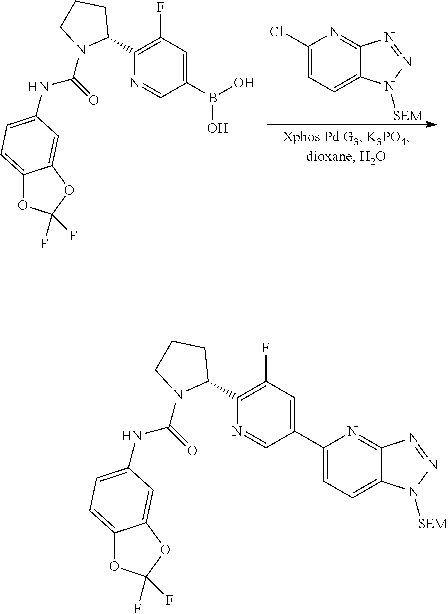 [SMILES: O=C(Nc1ccc2c(c1)OC(F)(F)O2)N1CCC[C@@H]1c1ncc(B(O)O)cc1F ; C[Si](C)(C)CCOCn1nnc2nc(Cl)ccc21 ; C[Si](C)(C)CCOCn1nnc2nc(-c3cnc([C@H]4CCCN4C(=O)Nc4ccc5c(c4)OC(F)(F)O5)c(F)c3)ccc21]

A solution of (R)-(6-(1-((2,2-difluorobenzo[d][1,3]dioxol-5-yl)carbamoyl)pyrrolidin-2-yl)-5-fluoropyridin-3-yl)boronic acid (45 mg, 0.110 mmol), 5-chloro-1-((2-(trimethylsilyl)ethoxy)methyl)-1H-[1,2,3]triazolo[4,5-b]pyridine (38 mg, 0.130 mmol), K₃PO₄ (47 mg, 0.22 mmol), Xphos Pd G₃ (9 mg, 10 μmol) and H₂O (0.4 mL) in 1,4-dioxane (2 mL) was stirred at 80° C. for overnight under nitrogen atmosphere. Upon completion, the mixture was cooled to room temperature and concentrated under reduced pressure. The crude residue was directly purified by silica gel column chromatography, eluted with PE/EA (1:1) to afford (R)—N-(2,2-difluorobenzo[d][1,3]dioxol-5-yl)-2-(3-fluoro-5-(1-((2-(trimethylsilyl)ethoxy)methyl)-1H-[1,2,3]triazolo[4,5-b]pyridin-5-yl)pyridin-2-yl)pyrrolidine-1-carboxamide (60 mg, 89% yield) as a yellow solid. LCMS (ESI) [M+H]⁺: 614.2.

### Step 5: (R)-2-(5-(1H-[1,2,3]triazolo[4,5-b]pyridin-5-yl)-3-fluoropyridin-2-yl)-N-(2,2-difluorobenzo[d][1,3]dioxol-5-yl)pyrrolidine-1-carboxamide

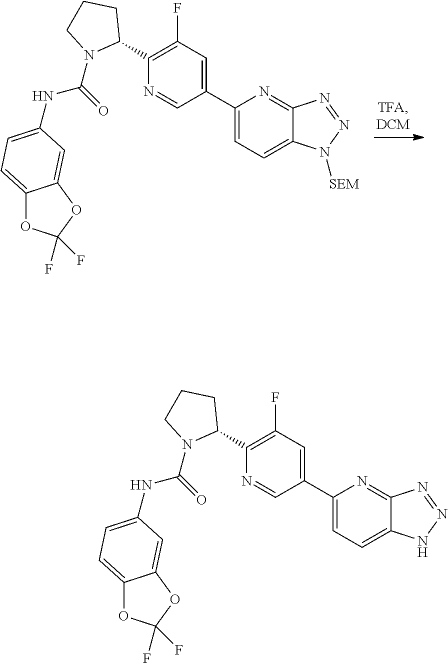 [SMILES: C[Si](C)(C)CCOCn1nnc2nc(-c3cnc([C@H]4CCCN4C(=O)Nc4ccc5c(c4)OC(F)(F)O5)c(F)c3)ccc21 ; O=C(Nc1ccc2c(c1)OC(F)(F)O2)N1CCC[C@@H]1c1ncc(-c2ccc3[nH]nnc3n2)cc1F]

To a mixture of (R)—N-(2,2-difluorobenzo[d][1,3]dioxol-5-yl)-2-(3-fluoro-5-(1-((2-(trimethylsilyl)ethoxy)methyl)-1H-[1,2,3]triazolo[4,5-b]pyridin-5-yl)pyridin-2-yl)pyrrolidine-1-carboxamide (60 mg, 90 μmol) in DCM (1 mL) at 0° C. was added TFA (1 mL) dropwise. The reaction mixture was warmed to room temperature and stirred for 2 hours. Upon completion, the resultant mixture was concentrated under reduced pressure. The crude residue was directly purified by Prep-HPLC (conditions: Column: Xselect CSH Prep C18, 30*150 mm 5 μm; Mobile Phase A: Water (0.1% FA), Mobile Phase B: ACN; Flow rate: 60 mL/min; Gradient (B %): 34% B to 47% B in 12 min; Wave Length: 254 nm/220 nm; RT1 (min): 11.13) to afford (R)-2-(5-(1H-[1,2,3]triazolo[4,5-b]pyridin-5-yl)-3-fluoropyridin-2-yl)-N-(2,2-difluorobenzo[d][1,3]dioxol-5-yl)pyrrolidine-1-carboxamide (Compound 286) (15.1 mg, 32% yield) as a white solid.

| Compound# | Characterization Data |
|---|---|
| 286 | LCMS (ESI) [M + H]⁺: 484.2 |
| | ¹H NMR (400 MHz, DMSO-d₆) δ 9.13 (s, 1H), 8.58 (d, J = 8.7 Hz, 1H), 8.50 (s, 1H), 8.35 (d, J = |
| | 11.2 Hz, 1H), 8.19-8.13 (m, 1H), 7.58 (s, 1H), 7.23-7.17 (m, 2H), 5.37-5.35 (m, 1H), 3.80- |
| | 3.78 (m, 1H), 3.76-3.62 (m, 1H), 2.40-2.35 (m, 1H), 2.33-2.15 (m, 2H), 2.07-1.94 (m, 1H). |

### Example 287: (S)-5-(4-(1-((5-fluoro-6-isopropylpyridin-3-yl)carbamoyl)-3,3-dimethylazetidine-2-carboxamido)phenyl)-6-methylpicolinic acid and (R)-5-(4-(1-((5-fluoro-6-isopropylpyridin-3-yl)carbamoyl)-3,3-dimethylazetidine-2-carboxamido)phenyl)-6-methylpicolinic acid

### Step 1: 5-fluoro-6-(prop-1-en-2-yl)pyridin-3-amine

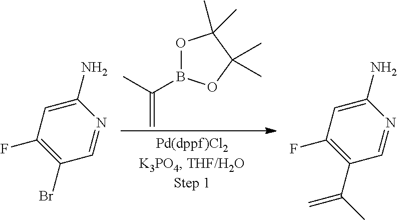

A solution of 6-bromo-5-fluoropyridin-3-amine (300 mg, 1.57 mmol), 4,4,5,5-tetramethyl-2-(prop-1-en-2-yl)-1,3,2-dioxaborolane (316 mg, 1.88 mmol), K₃PO₄ (1.00 g, 4.71 mmol), and Pd(dppf)Cl₂ (128 mg, 0.157 mmol) in THF (8.0 mL) and water (2.0 mL) was heated to 90° C. After 30 minutes, the mixture was concentrated under reduced pressure. The crude residue was directly purified by silica gel column chromatography, eluted with petroleum ether/ethyl acetate (2:1), to afford 5-fluoro-6-(prop-1-en-2-yl)pyridin-3-amine (180 mg, 75%) as a yellow oil. LCMS (ESI) [M+H]⁺: 153.1

### Step 2: 5-fluoro-6-isopropylpyridin-3-amine

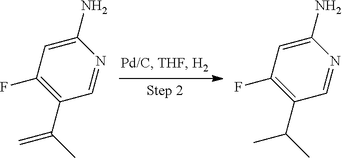

A solution of 5-fluoro-6-(prop-1-en-2-yl)pyridin-3-amine (180 mg, 1.18 mmol) and 10% palladium on carbon (150 mg) in THF (15.0 mL) was stirred at room temperature under an atmosphere of hydrogen. After 30 minutes, the mixture was filtered through celite. The filter cake was washed with dichloromethane (3×10 mL). The filtrate was concentrated under reduced pressure. The crude residue was purified by silica gel column chromatography, eluted with petroleum ether/ethyl acetate (2:1), to afford 5-fluoro-6-isopropylpyridin-3-amine (155 mg, 85%) as a yellow oil. LCMS (ESI) [M+H]⁺: 155.1

### Step 3: 5-(4-(1-((5-fluoro-6-isopropylpyridin-3-yl)carbamoyl)-3,3-dimethylazetidine-2-carboxamido)phenyl)-6-methylpicolinic acid

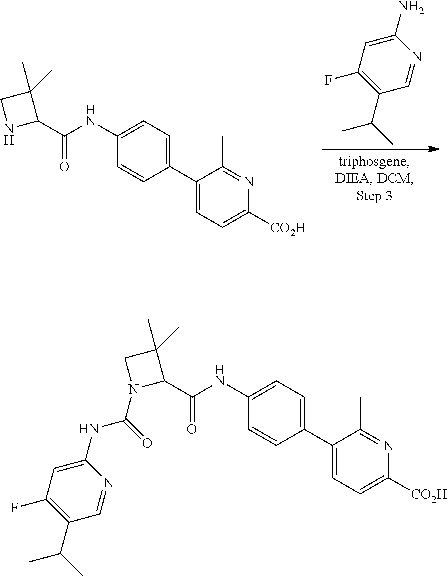 [SMILES: Cc1nc(C(=O)O)ccc1-c1ccc(NC(=O)C2NCC2(C)C)cc1 ; CC(C)c1cnc(N)cc1F ; Cc1nc(C(=O)O)ccc1-c1ccc(NC(=O)C2N(C(=O)Nc3cc(F)c(C(C)C)cn3)CC2(C)C)cc1]

To a stirred solution of 5-fluoro-6-isopropylpyridin-3-amine (150 mg, 0.973 mmol) and DIEA (628 mg, 4.86 mmol) in dichloromethane (6.0 mL) at 0° C. a solution of triphosgene (115 mg, 0.389 mmol) in dichloromethane (6.0 mL) was added dropwise over 10 minutes. After 5 min, the reaction was warmed to room temperature and 5-(4-(3,3-dimethylazetidine-2-carboxamido)phenyl)-6-methylpicolinic acid (330 mg, 0.973 mmol) was added. After stirring for an additional 2 hours, the reaction mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0%-100% over 30 min; detector: UV 254 nm) to afford 5-(4-(1-((5-fluoro-6-isopropylpyridin-3-yl)carbamoyl)-3,3-dimethylazetidine-2-carboxamido)phenyl)-6-methylpicolinic acid (82 mg, 16%) as an off-white solid. LCMS (ESI) [M+H]⁺: 520.2

### Step 4: (S)-5-(4-(1-((5-fluoro-6-isopropylpyridin-3-yl)carbamoyl)-3,3-dimethylazetidine-2-carboxamido)phenyl)-6-methylpicolinic acid and (R)-5-(4-(1-((5-fluoro-6-isopropylpyridin-3-yl)carbamoyl)-3,3-dimethylazetidine-2-carboxamido)phenyl)-6-methylpicolinic acid

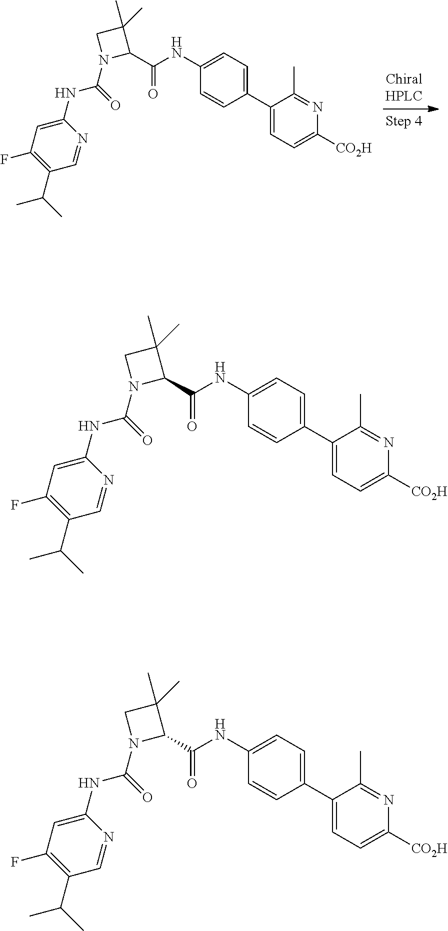 [SMILES: Cc1nc(C(=O)O)ccc1-c1ccc(NC(=O)C2N(C(=O)Nc3cc(F)c(C(C)C)cn3)CC2(C)C)cc1 ; Cc1nc(C(=O)O)ccc1-c1ccc(NC(=O)[C@H]2N(C(=O)Nc3cc(F)c(C(C)C)cn3)CC2(C)C)cc1 ; Cc1nc(C(=O)O)ccc1-c1ccc(NC(=O)[C@@H]2N(C(=O)Nc3cc(F)c(C(C)C)cn3)CC2(C)C)cc1]

5-(4-(1-((5-fluoro-6-isopropylpyridin-3-yl)carbamoyl)-3,3-dimethylazetidine-2-carboxamido)phenyl)-6-methylpicolinic acid (82 mg) was resolved by preparative chiral HPLC (conditions: column: CHIRAL ART Cellulose-SZ, 3*25 cm, 5 μm; mobile phase A: MTBE (0.1% formic acid), mobile phase B: methanol; flow rate: 40 mL/min; gradient (B %): isocratic, 50% B; detector: 220/254 nm; RT1 (min): 5.6; RT2 (min): 13.9) to afford (Compound 287a; Method BL, Peak 1, 0.63 min) (S)-5-(4-(1-((5-fluoro-6-isopropylpyridin-3-yl)carbamoyl)-3,3-dimethylazetidine-2-carboxamido)phenyl)-6-methylpicolinic acid (1^{st} peak, 26.2 mg, 32%) as a white solid, and (Compound 287b; Method BL, Peak 2, 1.63 min) (R)-5-(4-(1-((5-fluoro-6-isopropylpyridin-3-yl)carbamoyl)-3,3-dimethylazetidine-2-carboxamido)phenyl)-6-methylpicolinic acid (2^{nd} peak, 35.3 mg, 43%) as a white solid.

| Cmpd # | Characterization Data |
|---|---|
| 287a | LCMS (ESI) [M + H]⁺: 520.2 |
| | ¹H NMR (400 MHz, CD₃OD) δ 8.37 (d, J = 2.1 Hz, 1H), 8.06 (s, 1H), 7.90-7.81 (m, 2H), 7.77 (d, |
| | J = 8.0 Hz, 2H), 7.43-7.30 (m, 2H), 4.53 (d, J = 2.0 Hz, 1H), 3.89 (d, J = 7.4 Hz, 1H), 3.82 (d, J = |
| | 7.3 Hz, 1H), 3.32 (d, J = 4.2 Hz, 1H), 2.57 (s, 3H), 1.49 (s, 3H), 1.26 (dd, J = 6.9, 2.0 Hz, 9H). |
| 287b | LCMS (ESI) [M + H]⁺: 520.2 |
| | ¹H NMR (400 MHz, CD₃OD) δ 8.37 (d, J = 2.1 Hz, 1H), 8.06 (d, J = 7.9 Hz, 1H), 7.90-7.82 (m, |
| | 2H), 7.81-7.73 (m, 2H), 7.42-7.32 (m, 2H), 4.54 (d, J = 2.7 Hz, 1H), 3.89 (d, J = 7.4 Hz, 1H), |
| | 3.82 (d, J = 7.4 Hz, 1H), 3.31 (s, 1H), 2.57 (d, J = 5.0 Hz, 3H), 1.55-1.43 (m, 3H), 1.25 (dt, J = |
| | 7.0, 2.5 Hz, 9H). |

### Example 288: (S)-5-(4-(1-((6-isopropyl-5-methylpyridin-3-yl)carbamoyl)-3,3-dimethylazetidine-2-carboxamido)phenyl)-6-methylpicolinic acid and (R)-5-(4-(1-((6-isopropyl-5-methylpyridin-3-yl)carbamoyl)-3,3-dimethylazetidine-2-carboxamido)phenyl)-6-methylpicolinic acid

### Step 1: 5-methyl-6-(prop-1-en-2-yl)pyridin-3-amine

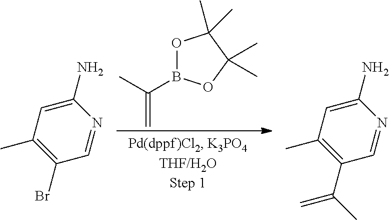

A solution of 6-bromo-5-methylpyridin-3-amine (500 mg, 2.67 mmol), 4,4,5,5-tetramethyl-2-(prop-1-en-2-yl)-1,3,2-dioxaborolane (674 mg, 4.01 mmol), Pd(dppf)Cl₂ (196 mg, 0.267 mmol) and K₃PO₄ (1.70 g, 8.02 mmol) in a solution of 1,4-dioxane (10.0 mL) and water (2.0 mL) was heated to 80° C. for 1 hour. Upon completion, the reaction mixture was diluted with water (60 mL) and extracted with ethyl acetate (3×80 mL). The combined organic layers were concentrated under reduced pressure. The crude residue was purified by silica gel column chromatography, eluted with petroleum ether/ethyl acetate (1:3), to afford 5-methyl-6-(prop-1-en-2-yl)pyridin-3-amine (310 mg, 78%) as a brown oil. LCMS (ESI) [M+H]⁺: 149.1

### Step 2: 6-isopropyl-5-methylpyridin-3-amine

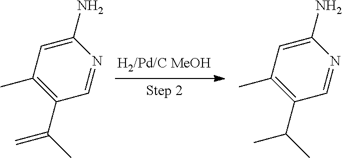

A solution of 5-methyl-6-(prop-1-en-2-yl)pyridin-3-amine (310 mg, 2.09 mmol) and 10% palladium on carbon (266 mg) in methanol (20 mL) was stirred under an atmosphere of hydrogen (1 atm) for 1 hour. Upon completion, the resultant mixture was filtered through a celite. The filtrate was concentrated under reduced pressure to afford 6-isopropyl-5-methylpyridin-3-amine (310 mg, crude) as a tan oil, which was used directly in the next step without further purification. LCMS (ESI) [M+H]⁺: 151.1

### Step 3: 5-(4-(1-((6-isopropyl-5-methylpyridin-3-yl)carbamoyl)-3,3-dimethylazetidine-2-carboxamido)phenyl)-6-methylpicolinic acid

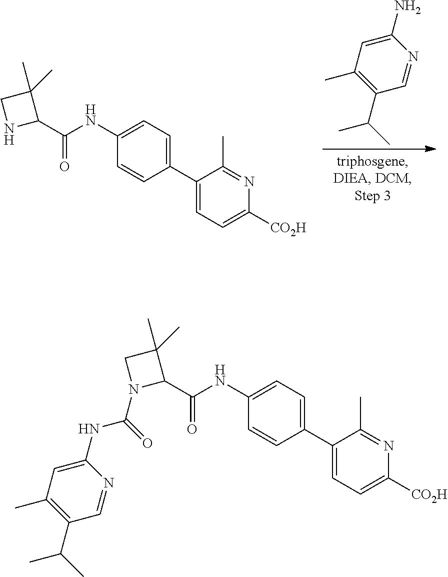 [SMILES: Cc1nc(C(=O)O)ccc1-c1ccc(NC(=O)C2NCC2(C)C)cc1 ; Cc1cc(N)ncc1C(C)C ; Cc1cc(NC(=O)N2CC(C)(C)C2C(=O)Nc2ccc(-c3ccc(C(=O)O)nc3C)cc2)ncc1C(C)C]

To a stirred solution of 6-isopropyl-5-methylpyridin-3-amine (120 mg, 0.799 mmol) and DIEA (275 mg, 2.13 mmol) in dichloromethane (1.0 mL) at 0° C. were added 5-(4-(3,3-dimethylazetidine-2-carboxamido)phenyl)-6-methylpicolinic acid (90.4 mg, 0.266 mmol) and triphosgene (79.0 mg, 0.266 mmol). The resultant mixture was warmed to room temperature and stirred for 1 hour. Upon completion, the mixture was concentrated under reduced pressure. The crude residue was directly purified by preparative HPLC (conditions: column: XSelect CSH Fluoro Phenyl 30*150 mm, 5 μm; mobile phase A: water (0.1% formic acid), mobile phase B: MeCN; flow rate: 60 mL/min; gradient (B %): 2%-15% B over 15 min; detector: 254 nm/220 nm; RT: 10.7 min) to afford 5-(4-(1-((6-isopropyl-5-methylpyridin-3-yl)carbamoyl)-3,3-dimethylazetidine-2-carboxamido)phenyl)-6-methylpicolinic acid (32 mg, 23%) as a white solid. LCMS (ESI) [M+H]⁺: 516.2.

### Step 4: (S)-5-(4-(1-((6-isopropyl-5-methylpyridin-3-yl)carbamoyl)-3,3-dimethylazetidine-2-carboxamido)phenyl)-6-methylpicolinic acid and (R)-5-(4-(1-((6-isopropyl-5-methylpyridin-3-yl)carbamoyl)-3,3-dimethylazetidine-2-carboxamido)phenyl)-6-methylpicolinic acid

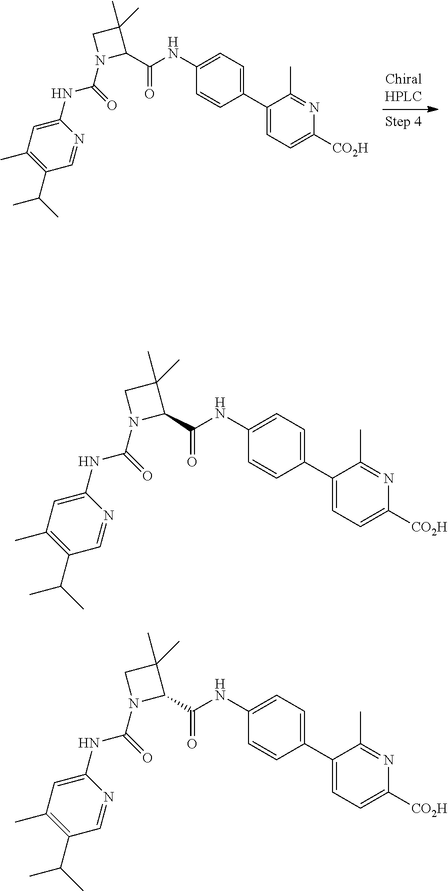 [SMILES: Cc1cc(NC(=O)N2CC(C)(C)C2C(=O)Nc2ccc(-c3ccc(C(=O)O)nc3C)cc2)ncc1C(C)C ; Cc1cc(NC(=O)N2CC(C)(C)[C@H]2C(=O)Nc2ccc(-c3ccc(C(=O)O)nc3C)cc2)ncc1C(C)C ; Cc1cc(NC(=O)N2CC(C)(C)[C@@H]2C(=O)Nc2ccc(-c3ccc(C(=O)O)nc3C)cc2)ncc1C(C)C]

5-(4-(1-((6-isopropyl-5-methylpyridin-3-yl)carbamoyl)-3,3-dimethylazetidine-2-carboxamido)phenyl)-6-methylpicolinic acid (32 mg) was resolved by preparative chiral HPLC (conditions: column: CHIRAL ART Cellulose-SZ, 3*25 cm, 5 μm; mobile phase A: MTBE (0.1% formic acid), mobile phase B: methanol; flow rate: 40 mL/min; gradient (B %): isocratic, 60% B; detector: 220/254 nm; RT1 (min): 6.2; RT2 (min): 26.7) to afford (Compound 288a; Method BO, Peak 1, 0.93 min) (S)-5-(4-(1-((6-isopropyl-5-methylpyridin-3-yl)carbamoyl)-3,3-dimethylazetidine-2-carboxamido)phenyl)-6-methylpicolinic acid (1^{st} peak, 10.5 mg, 33%) as a white solid, and (Compound 288b; Method BO, Peak 2, 2.13 min) (R)-5-(4-(1-((6-isopropyl-5-methylpyridin-3-yl)carbamoyl)-3,3-dimethylazetidine-2-carboxamido)phenyl)-6-methylpicolinic acid (2^{nd} peak, 8.7 mg, 27%) as a white solid.

| Cmpd # | Characterization Data |
|---|---|
| 288a | LCMS (ESI) [M + H]⁺: 516.3 |
| | ¹H NMR (400 MHz, CD₃OD) δ 8.51 (d, J = 2.4 Hz, 1H), 8.07 (d, J = 7.9 Hz, 1H), 7.86 (d, J = 7.8 |
| | Hz, 1H), 7.82 (d, J = 2.4 Hz, 1H), 7.77 (d, J = 8.2 Hz, 2H), 7.40 (d, J = 8.2 Hz, 2H), 4.53 (s, 1H), |
| | 3.89 (d, J = 7.3 Hz, 1H), 3.81 (d, J = 7.4 Hz, 1H), 3.29 (d, J = 8.2 Hz, 1H), 2.58 (s, 3H), 2.37 (s, |
| | 3H), 1.50 (s, 3H), 1.30-1.23 (m, 9H). |
| 288b | LCMS (ESI) [M + H]⁺: 516.3 |
| | ¹H NMR (400 MHz, CD₃OD) δ 8.44 (d, J = 2.5 Hz, 1H), 8.05 (s, 1H), 7.85 (d, J = 7.5 Hz, 1H), 7.81- |
| | 7.73 (m, 3H), 7.39 (d, J = 8.0 Hz, 2H), 4.52 (s, 1H), 3.88 (d, J = 7.3 Hz, 1H), 3.81 (d, J = 7.4 Hz, |
| | 1H), 3.27 (d, J = 6.8 Hz, 1H), 2.57 (s, 3H), 2.34 (s, 3H), 1.49 (s, 3H), 1.30-1.21 (m, 9H). |

### Example 289: (S)-5-(4-(3,3-dimethyl-1-((4-methyl-5-(trifluoromethyl)pyridin-2-yl)carbamoyl)azetidine-2-carboxamido)phenyl)-6-methylpicolinic acid and (R)-5-(4-(3,3-dimethyl-1-((4-methyl-5-(trifluoromethyl)pyridin-2-yl)carbamoyl)azetidine-2-carboxamido)phenyl)-6-methylpicolinic acid

### Step 1: 4-methyl-5-(trifluoromethyl)pyridin-2-amine

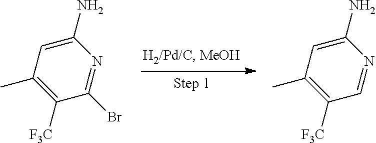

A mixture of 6-bromo-4-methyl-5-(trifluoromethyl)pyridin-2-amine (1.00 g, 3.91 mmol) and 10% palladium on carbon (280 mg) in methanol (10.0 mL) was stirred under hydrogen (1 atm) overnight. Upon completion, the mixture was filtered through celite. The filter cake was washed with methanol (3×45 mL). The filtrate was concentrated under reduced pressure to afford crude 4-methyl-5-(trifluoromethyl)pyridin-2-amine (0.300 g, crude) as a yellow oil, which was used directly in the next step without further purification. LCMS (ESI) [M+H]⁺: 177.0.

### Step 2: 5-(4-(3,3-dimethyl-1-((4-methyl-5-(trifluoromethyl)pyridin-2-yl)carbamoyl)azetidine-2-carboxamido)phenyl)-6-methylpicolinic acid

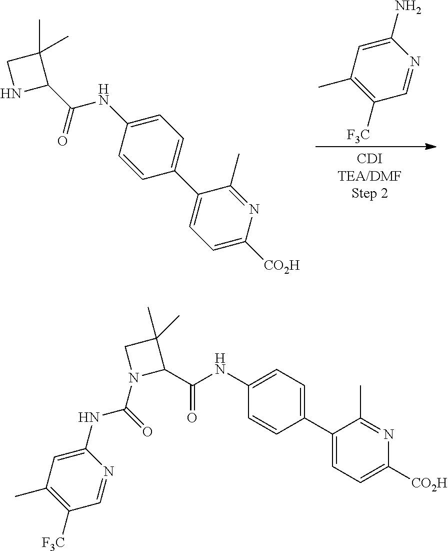 [SMILES: Cc1nc(C(=O)O)ccc1-c1ccc(NC(=O)C2NCC2(C)C)cc1 ; Cc1cc(N)ncc1C(F)(F)F ; Cc1cc(NC(=O)N2CC(C)(C)C2C(=O)Nc2ccc(-c3ccc(C(=O)O)nc3C)cc2)ncc1C(F)(F)F]

A solution of 4-methyl-5-(trifluoromethyl)pyridin-2-amine (300 mg, 1.70 mmol), 5-(4-(3,3-dimethylazetidine-2-carboxamido)phenyl)-6-methylpicolinic acid (578 mg, 1.70 mmol), carbonyldiimidazole (828 mg, 5.11 mmol), and triethylamine (517 mg, 5.11 mmol) in DMF (3.0 mL) was heated to 70° C. for 1 hour. Upon completion, the mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0%-100% over 40 min; detector: 254 nm) to afford 5-(4-(3,3-dimethyl-1-((4-methyl-5-(trifluoromethyl)pyridin-2-yl)carbamoyl)azetidine-2-carboxamido)phenyl)-6-methylpicolinic acid (70 mg, 8%) as a white solid. LCMS (ESI) [M+H]⁺: 542.1

### Step 3: (S)-5-(4-(3,3-dimethyl-1-((4-methyl-5-(trifluoromethyl)pyridin-2-yl)carbamoyl)azetidine-2-carboxamido)phenyl)-6-methylpicolinic acid and (R)-5-(4-(3,3-dimethyl-1-((4-methyl-5-(trifluoromethyl)pyridin-2-yl)carbamoyl)azetidine-2-carboxamido)phenyl)-6-methylpicolinic acid

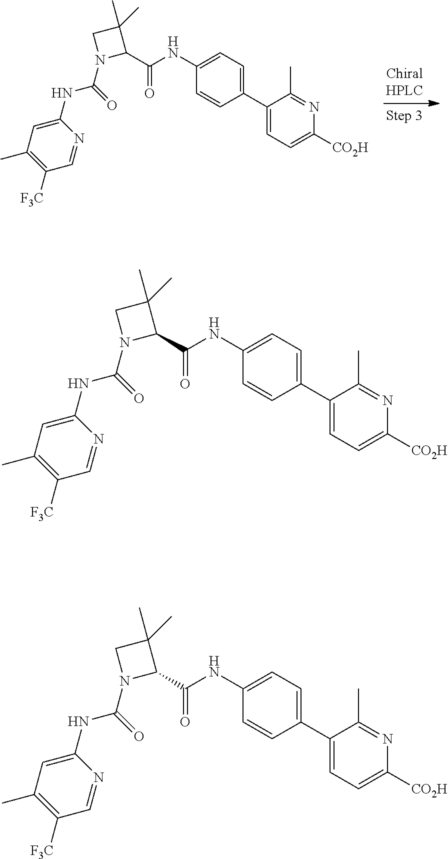 [SMILES: Cc1cc(NC(=O)N2CC(C)(C)C2C(=O)Nc2ccc(-c3ccc(C(=O)O)nc3C)cc2)ncc1C(F)(F)F ; Cc1cc(NC(=O)N2CC(C)(C)[C@H]2C(=O)Nc2ccc(-c3ccc(C(=O)O)nc3C)cc2)ncc1C(F)(F)F ; Cc1cc(NC(=O)N2CC(C)(C)[C@@H]2C(=O)Nc2ccc(-c3ccc(C(=O)O)nc3C)cc2)ncc1C(F)(F)F]

-(4-(3,3-dimethyl-1-((4-methyl-5-(trifluoromethyl)pyridin-2-yl)carbamoyl)azetidine-2-carboxamido)phenyl)-6-methylpicolinic acid (70 mg) was resolved by preparative chiral HPLC (conditions: column: CHIRAL ART Cellulose-SZ, 3*25 cm, 5 μm; mobile phase A: MTBE (0.1% formic acid), mobile phase B: methanol; flow rate: 40 mL/min; gradient (B %): isocratic, 50% B; detector: 220/254 nm; RT1 (min): 7.2; RT2 (min): 27.0) to afford (Compound 289a; Method BL, Peak 1, 0.61 min) (S)-5-(4-(3,3-dimethyl-1-((4-methyl-5-(trifluoromethyl)pyridin-2-yl)carbamoyl)azetidine-2-carboxamido)phenyl)-6-methylpicolinic acid (1^{st} peak, 10.4 mg, 15%) as a white solid, and (Compound 289b; Method BL, Peak 2, 3.18 min) (R)-5-(4-(3,3-dimethyl-1-((4-methyl-5-(trifluoromethyl)pyridin-2-yl)carbamoyl)azetidine-2-carboxamido)phenyl)-6-methylpicolinic acid (2^{nd} peak, 9.8 mg, 14%) as a white solid.

| Cmpd # | Characterization Data |
|---|---|
| 289a | LCMS (ESI) [M + H]⁺: 502.3 |
| | ¹H NMR (400 MHz, CD₃OD) δ 8.42 (s, 1H), 8.07 (d, J = 7.9 Hz, 1H), 7.99 (s, 1H), 7.87 (d, J = |
| | 7.8 Hz, 1H), 7.78 (d, J = 8.2 Hz, 2H), 7.40 (d, J = 8.3 Hz, 2H), 4.57 (s, 1H), 3.92 (d, J = 7.5 Hz, |
| | 1H), 3.84 (d, J = 7.5 Hz, 1H), 2.58 (s, 3H), 2.46 (s, 3H), 1.50 (s, 3H), 1.27 (s, 3H). |
| 289b | LCMS (ESI) [M + H]⁺: 502.2 |
| | ¹H NMR (400 MHz, CD₃OD) δ 8.42 (s, 1H), 8.07 (s, 1H), 7.99 (s, 1H), 7.86 (d, J = 7.4 Hz, 1H), |
| | 7.78 (d, J = 7.9 Hz, 2H), 7.40 (d, J = 8.1 Hz, 2H), 4.57 (s, 1H), 3.92 (d, J = 7.4 Hz, 1H), 3.84 (d, |
| | J = 7.5 Hz, 1H), 2.58 (s, 3H), 2.46 (s, 3H), 1.50 (s, 3H), 1.27 (s, 3H). |

### Example 290: (S)-5-(4-(1-((3-fluoro-4-(trifluoromethyl)phenyl)carbamoyl)-3,3-dimethylazetidine-2-carboxamido)phenyl)-6-methylpicolinic acid and (R)-5-(4-(1-((3-fluoro-4-(trifluoromethyl)phenyl)carbamoyl)-3,3-dimethylazetidine-2-carboxamido)phenyl)-6-methylpicolinic acid

### Step 1: 5-(4-(1-((3-fluoro-4-(trifluoromethyl)phenyl)carbamoyl)-3,3-dimethylazetidine-2-carboxamido)phenyl)-6-methylpicolinic acid

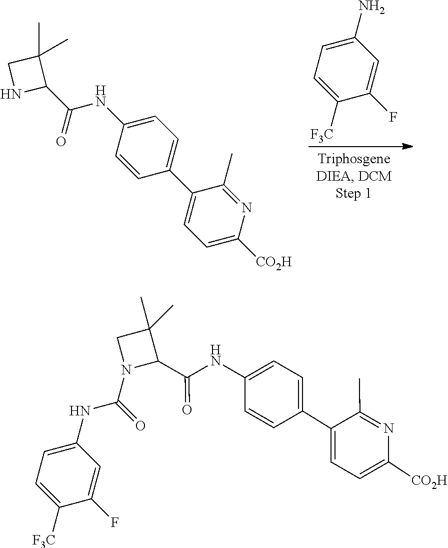 [SMILES: Cc1nc(C(=O)O)ccc1-c1ccc(NC(=O)C2NCC2(C)C)cc1 ; Nc1ccc(C(F)(F)F)c(F)c1 ; Cc1nc(C(=O)O)ccc1-c1ccc(NC(=O)C2N(C(=O)Nc3ccc(C(F)(F)F)c(F)c3)CC2(C)C)cc1]

A solution of 3-fluoro-4-(trifluoromethyl)aniline (39.6 mg, 0.220 mmol), 5-(4-(3,3-dimethylazetidine-2-carboxamido)phenyl)-6-methylpicolinic acid (50.0 mg, 0.147 mmol), DIEA (95.2 mg, 0.735 mmol) and triphosgene (21.9 mg, 0.073 mmol) in dichloromethane (2.0 mL) was stirred at 25° C. for 1 hour. Upon completion, the mixture was concentrated under reduced pressure. The crude residue was directly purified by reversed phase flash chromatography (conditions: column: C18 silica gel; mobile phase A: water (0.1% formic acid); mobile phase B: MeCN; gradient (B %): 0%-100% over 25 min; detector: UV 254/220 nm) to afford 5-(4-(1-((3-fluoro-4-(trifluoromethyl)phenyl)carbamoyl)-3,3-dimethylazetidine-2-carboxamido)phenyl)-6-methylpicolinic acid (52 mg, 65%) as an off-white solid. LCMS (ESI) [M+H]⁺: 545.5

### Step 2: (S)-5-(4-(1-((3-fluoro-4-(trifluoromethyl)phenyl)carbamoyl)-3,3-dimethylazetidine-2-carboxamido)phenyl)-6-methylpicolinic acid and (R)-5-(4-(1-((3-fluoro-4-(trifluoromethyl)phenyl)carbamoyl)-3,3-dimethylazetidine-2-carboxamido)phenyl)-6-methylpicolinic acid

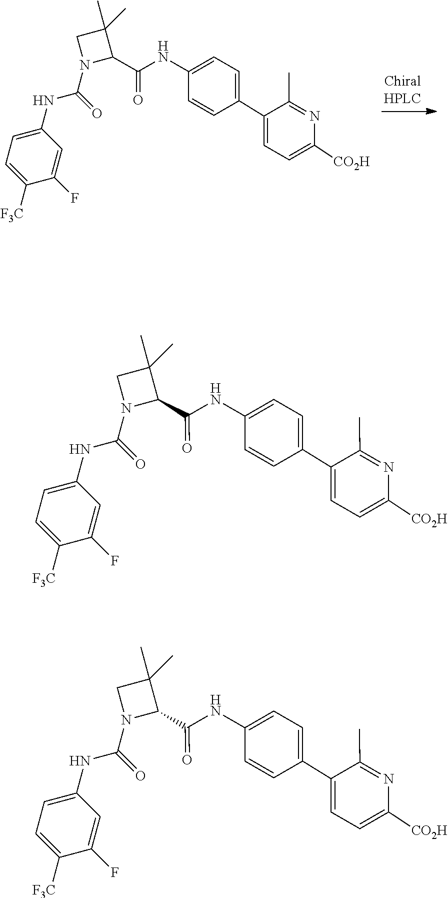 [SMILES: Cc1nc(C(=O)O)ccc1-c1ccc(NC(=O)C2N(C(=O)Nc3ccc(C(F)(F)F)c(F)c3)CC2(C)C)cc1 ; Cc1nc(C(=O)O)ccc1-c1ccc(NC(=O)[C@H]2N(C(=O)Nc3ccc(C(F)(F)F)c(F)c3)CC2(C)C)cc1 ; Cc1nc(C(=O)O)ccc1-c1ccc(NC(=O)[C@@H]2N(C(=O)Nc3ccc(C(F)(F)F)c(F)c3)CC2(C)C)cc1]

5-(4-(1-((3-fluoro-4-(trifluoromethyl)phenyl)carbamoyl)-3,3-dimethylazetidine-2-carboxamido)phenyl)-6-methylpicolinic acid (50 mg) was resolved by preparative chiral HPLC (conditions: column: CHIRAL ART Cellulose-SZ, 3*25 cm, 5 μm; mobile phase A: MTBE (0.1% formic acid), mobile phase B: methanol; flow rate: 40 mL/min; gradient (B %): isocratic, 50% B; detector: 220/254 nm; RT1 (min): 6.4; RT2 (min): 12.3) to afford (Compound 290a; Method BL, Peak 1, 0.58 min) (S)-5-(4-(1-((3-fluoro-4-(trifluoromethyl)phenyl)carbamoyl)-3,3-dimethylazetidine-2-carboxamido)phenyl)-6-methylpicolinic acid (1^{st} peak, 22.2 mg, 44%) as a white solid, and (Compound 290b; Method BL, Peak 2, 1.67 min) (R)-5-(4-(1-((3-fluoro-4-(trifluoromethyl)phenyl)carbamoyl)-3,3-dimethylazetidine-2-carboxamido)phenyl)-6-methylpicolinic acid (2^{nd} peak, 18.2 mg, 36%) as a white solid.

| Cmpd # | Characterization Data |
|---|---|
| 290a | LCMS (ESI) [M + H]⁺: 545.2 |
| | ¹H NMR (400 MHz, CD₃OD) δ 8.05 (d, J = 7.8 Hz, 1H), 7.85 (d, J = 7.8 Hz, 1H), 7.77 (d, J = 8.2 |
| | Hz, 2H), 7.66 (dd, J = 13.7, 2.0 Hz, 1H), 7.53 (t, J = 8.5 Hz, 1H), 7.38 (d, J = 8.6 Hz, 3H), 4.54 (s, |
| | 1H), 3.91 (d, J = 7.4 Hz, 1H), 3.82 (d, J = 7.4 Hz, 1H), 2.57 (s, 3H), 1.50 (s, 3H), 1.27 (s, 3H). |
| 290b | LCMS (ESI) [M + H]⁺: 545.2 |
| | ¹H NMR (400 MHz, CD₃OD) δ 8.06 (d, J = 7.9 Hz, 1H), 7.85 (d, J = 7.8 Hz, 1H), 7.77 (d, J = 8.4 |
| | Hz, 2H), 7.66 (dd, J = 13.5, 1.9 Hz, 1H), 7.53 (t, J = 8.5 Hz, 1H), 7.39 (d, J = 8.6 Hz, 3H), 4.54 (s, |
| | 1H), 3.91 (d, J = 7.4 Hz, 1H), 3.82 (d, J = 7.5 Hz, 1H), 2.58 (s, 3H), 1.50 (s, 3H), 1.27 (s, 3H). |

### Example 291: (S)-5-(4-(1-((5-isopropyl-4-methylpyridin-2-yl)carbamoyl)-3,3-dimethylazetidine-2-carboxamido)phenyl)-6-methylpicolinic acid and (R)-5-(4-(1-((5-isopropyl-4-methylpyridin-2-yl)carbamoyl)-3,3-dimethylazetidine-2-carboxamido)phenyl)-6-methylpicolinic acid

### Step 1: 4-methyl-5-(prop-1-en-2-yl)pyridin-2-amine

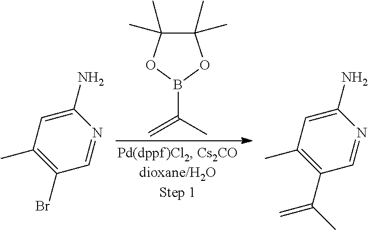

A solution of 5-bromo-4-methylpyridin-2-amine (1.00 g, 5.35 mmol), 4,4,5,5-tetramethyl-2-(prop-1-en-2-yl)-1,3,2-dioxaborolane (1.80 g, 10.6 mmol), Pd(dppf)Cl₂ (0.390 g, 0.535 mmol) and cesium carbonate (5.23 g, 16.0 mmol) in 1,4-dioxane (18.0 mL) and water (2.0 mL) was heated to 80° C. for 1 hour. Upon completion, the mixture was diluted with water (60 mL) and extracted with ethyl acetate (3×80 mL). The combined organic layers were concentrated under reduced pressure. The crude residue was purified by silica gel column chromatography, eluted with petroleum ether/ethyl acetate (1:3) to afford 4-methyl-5-(prop-1-en-2-yl)pyridin-2-amine (440 mg, 56%) as a brown oil. LCMS (ESI) [M+H]⁺: 149.1

### Step 2: 5-isopropyl-4-methylpyridin-2-amine

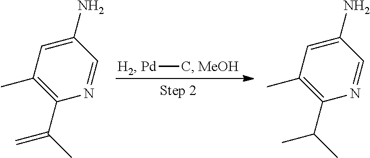

A solution of 4-methyl-5-(prop-1-en-2-yl)pyridin-2-amine (440 mg, 2.97 mmol) and 10% palladium on carbon (316 mg) in methanol (20 mL) was stirred under hydrogen atmosphere (1 atm) for 1 hour. Upon completion, the mixture was filtered through celite. The filtrate was concentrated under reduced pressure to afford 6-isopropyl-5-methylpyridin-3-amine (370 mg, crude) as a colorless oil, which was used directly in the next step without further purification. LCMS (ESI) [M+H]⁺: 151.1

### Step 3: 5-(4-(1-((5-isopropyl-4-methylpyridin-2-yl)carbamoyl)-3,3-dimethylazetidine-2-carboxamido)phenyl)-6-methylpicolinic acid

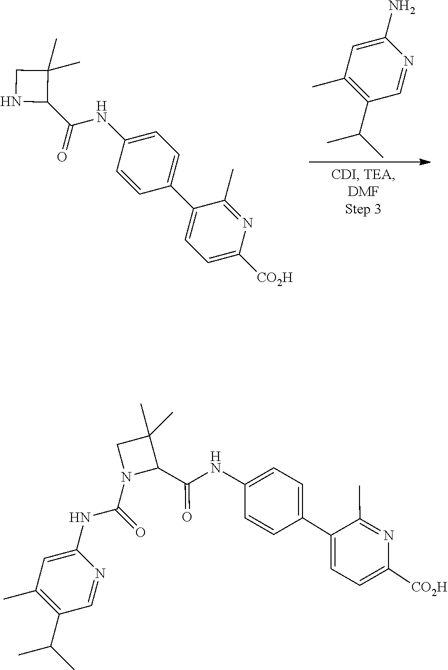 [SMILES: Cc1nc(C(=O)O)ccc1-c1ccc(NC(=O)C2NCC2(C)C)cc1 ; Cc1cc(N)ncc1C(C)C ; Cc1cc(NC(=O)N2CC(C)(C)C2C(=O)Nc2ccc(-c3ccc(C(=O)O)nc3C)cc2)ncc1C(C)C]

To a stirred solution of 5-isopropyl-4-methylpyridin-2-amine (100 mg, 0.666 mmol) carbonyldiimidazole (144 mg, 0.888 mmol) and triethylamine (89.8 mg, 0.888 mmol) in DMF (2.0 mL) at room temperature was added 5-(4-(3,3-dimethylazetidine-2-carboxamido)phenyl)-6-methylpicolinic acid (75.3 mg, 0.222 mmol). The reaction mixture was heated to 50° C. for 1 hour. Upon completion, the mixture was concentrated under reduced pressure. The crude residue was purified by reversed phase chromatography (conditions: column: Xbridge Phenyl OBD Column, 30*150 mm 5 μm; mobile phase A: water (0.1% formic acid); mobile phase B: MeCN; flow rate: 60 mL/min; gradient (B %): 5%-25% B over 15 min; detector: 254/220 nm; RT (min): 10.3 to afford 5-(4-(1-((5-isopropyl-4-methylpyridin-2-yl)carbamoyl)-3,3-dimethylazetidine-2-carboxamido)phenyl)-6-methylpicolinic acid (32 mg, 27%) as a white solid. LCMS (ESI) [M+H]⁺: 516.2.

### Step 4: (S)-5-(4-(1-((5-isopropyl-4-methylpyridin-2-yl)carbamoyl)-3,3-dimethylazetidine-2-carboxamido)phenyl)-6-methylpicolinic acid and (R)-5-(4-(1-((5-isopropyl-4-methylpyridin-2-yl)carbamoyl)-3,3-dimethylazetidine-2-carboxamido)phenyl)-6-methylpicolinic acid

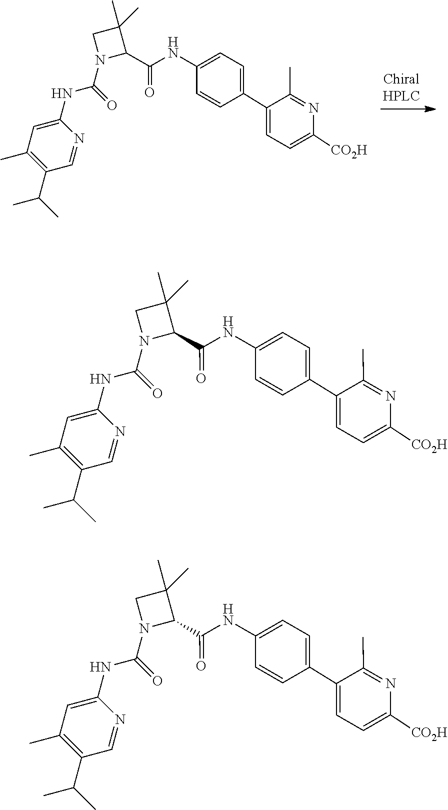 [SMILES: Cc1cc(NC(=O)N2CC(C)(C)C2C(=O)Nc2ccc(-c3ccc(C(=O)O)nc3C)cc2)ncc1C(C)C ; Cc1cc(NC(=O)N2CC(C)(C)[C@H]2C(=O)Nc2ccc(-c3ccc(C(=O)O)nc3C)cc2)ncc1C(C)C ; Cc1cc(NC(=O)N2CC(C)(C)[C@@H]2C(=O)Nc2ccc(-c3ccc(C(=O)O)nc3C)cc2)ncc1C(C)C]

5-(4-(1-((5-isopropyl-4-methylpyridin-2-yl)carbamoyl)-3,3-dimethylazetidine-2-carboxamido)phenyl)-6-methylpicolinic acid (32 mg) was resolved by preparative chiral HPLC (conditions: column: CHIRAL ART Cellulose-SZ, 3*25 cm, 5 μm; mobile phase A: MTBE (0.1% formic acid), mobile phase B: methanol; flow rate: 40 mL/min; gradient (B %): isocratic, 30% B; detector: 220/254 nm; RT1 (min): 10.2; RT2 (min): 42.5) to afford (Compound 291a; Method BP, Peak 1, 0.93 min) (S)-5-(4-(1-((5-isopropyl-4-methylpyridin-2-yl)carbamoyl)-3,3-dimethylazetidine-2-carboxamido)phenyl)-6-methylpicolinic acid (1^{st} peak, 10.5 mg, 32%) as a white solid, and (Compound 291b; Method BP, Peak 2, 2.54 min) (R)-5-(4-(1-((5-isopropyl-4-methylpyridin-2-yl)carbamoyl)-3,3-dimethylazetidine-2-carboxamido)phenyl)-6-methylpicolinic acid (2^{nd} peak, 16.6 mg, 51%) as a white solid.

| Cmpd # | Characterization Data |
|---|---|
| 291a | LCMS (ESI) [M + H]⁺: 516.3 |
| | ¹H NMR (400 MHz, CD₃OD) δ 8.12-8.00 (m, 2H), 7.86 (d, J = 7.8 Hz, 1H), 7.78 (d, J = 8.5 Hz, |
| | 2H), 7.50 (s, 1H), 7.40 (d, J = 8.3 Hz, 2H), 4.58 (s, 1H), 3.91 (d, J = 7.5 Hz, 1H), 3.85 (d, J = 7.5 |
| | Hz, 1H), 3.23-3.12 (m, 1H), 2.58 (s, 3H), 2.44 (s, 3H), 1.51 (s, 3H), 1.28 (d, J = 6.9 Hz, 9H). |
| 291b | LCMS (ESI) [M + H]⁺: 516.3 |
| | ¹H NMR (400 MHz, CD₃OD) δ 8.10-7.96 (m, 2H), 7.85 (dd, J = 15.6, 5.9 Hz, 1H), 7.81-7.73 |
| | (m, 2H), 7.69-7.64 (m, 1H), 7.45-7.33 (m, 2H), 4.54 (s, 1H), 3.87 (d, J = 7.3 Hz, 1H), 3.81 (d, J = |
| | 7.3 Hz, 1H), 3.17-3.08 (m, 1H), 2.57 (s, 3H), 2.35 (s, 3H), 1.49 (s, 3H), 1.26 (d, J = 6.7 Hz, 9H). |

### Example 292: (S)-4′-(1-((3-fluoro-4-(1-(trifluoromethyl)cyclopropyl)phenyl)carbamoyl)-3,3-dimethylazetidine-2-carboxamido)-[1,1′-biphenyl]-4-carboxylic acid and (R)-4′-(1-((3-fluoro-4-(1-(trifluoromethyl)cyclopropyl)phenyl)carbamoyl)-3,3-dimethylazetidine-2-carboxamido)-[1,1′-biphenyl]-4-carboxylic acid

### Step 1: methyl 4′-(3,3-dimethylazetidine-2-carboxamido)-[1,1′-biphenyl]-4-carboxylate

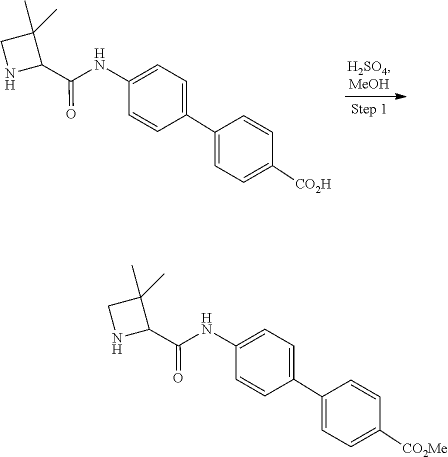 [SMILES: CC1(C)CNC1C(=O)Nc1ccc(-c2ccc(C(=O)O)cc2)cc1 ; COC(=O)c1ccc(-c2ccc(NC(=O)C3NCC3(C)C)cc2)cc1]

To a solution of 4′-(3,3-dimethylazetidine-2-carboxamido)-[1,1′-biphenyl]-4-carboxylic acid (300 mg, 0.92 mmol) in methanol (3.0 mL) at room temperature was added sulfuric acid (1.0 mL). After stirring overnight, the reaction mixture was cooled to 0° C., diluted with ice water (10 mL) and adjusted pH=7 by the portion-wise addition of solid sodium bicarbonate. The resulting mixture was extracted with dichloromethane (3×10 mL). The combined organic layers were concentrated under reduced pressure. The crude residue was purified by reversed phase column chromatography (conditions: column: C18 silica gel; mobile phase A: water (0.1% NH₄HCO₃); mobile phase B: MeCN; gradient (B %): 0%-100% over 25 min; detector: 254 nm) to afford methyl 4′-(3,3-dimethylazetidine-2-carboxamido)-[1,1′-biphenyl]-4-carboxylate (260 mg, 83%) as a white solid. LCMS (ESI) [M+H]⁺: 339.2

### Step 2: methyl 4′-(1-((3-fluoro-4-(1-(trifluoromethyl)cyclopropyl)phenyl)carbamoyl)-3,3-dimethylazetidine-2-carboxamido)-[1,1′-biphenyl]-4-carboxylate

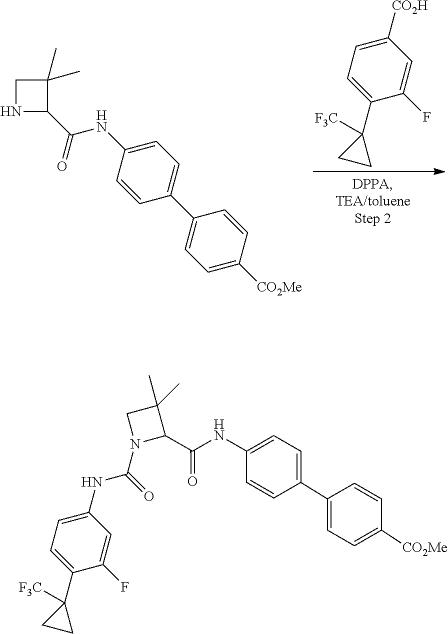 [SMILES: COC(=O)c1ccc(-c2ccc(NC(=O)C3NCC3(C)C)cc2)cc1 ; O=C(O)c1ccc(C2(C(F)(F)F)CC2)c(F)c1 ; COC(=O)c1ccc(-c2ccc(NC(=O)C3N(C(=O)Nc4ccc(C5(C(F)(F)F)CC5)c(F)c4)CC3(C)C)cc2)cc1]

To a solution of 3-fluoro-4-(1-(trifluoromethyl)cyclopropyl)benzoic acid (176 mg, 0.700 mmol) and triethylamine (179 mg, 1.77 mmol) in toluene (3 mL) was added diphenylphosphoryl azide (325 mg, 1.18 mmol) followed by methyl 4′-(3,3-dimethylazetidine-2-carboxamido)-[1,1′-biphenyl]-4-carboxylate (200 mg, 0.59 mmol). The resultant mixture was heated to 80° C. and stirred for 2 hours. Upon completion, the mixture was cooled to ambient temperature and concentrated under reduced pressure. The crude residue was directly purified by silica gel chromatography, eluted with dichloromethane/methanol (10:1) to afford methyl 4′-(1-((3-fluoro-4-(1-(trifluoromethyl)cyclopropyl)phenyl)carbamoyl)-3,3-dimethylazetidine-2-carboxamido)-[1,1′-biphenyl]-4-carboxylate (130 mg, 37%) as a yellow semi-solid. LCMS (ESI) [M+H]⁺: 584.3

### Step 3: 4′-(1-((3-fluoro-4-(1-(trifluoromethyl)cyclopropyl)phenyl)carbamoyl)-3,3-dimethylazetidine-2-carboxamido)-[1,1′-biphenyl]-4-carboxylic acid

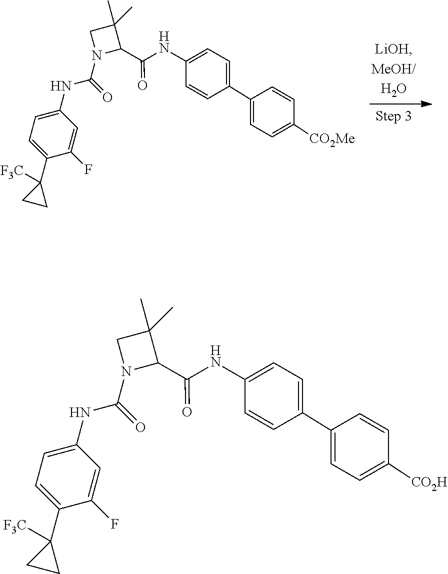 [SMILES: COC(=O)c1ccc(-c2ccc(NC(=O)C3N(C(=O)Nc4ccc(C5(C(F)(F)F)CC5)c(F)c4)CC3(C)C)cc2)cc1 ; CC1(C)CN(C(=O)Nc2ccc(C3(C(F)(F)F)CC3)c(F)c2)C1C(=O)Nc1ccc(-c2ccc(C(=O)O)cc2)cc1]

To a solution of methyl 4′-(1-((3-fluoro-4-(1-(trifluoromethyl)cyclopropyl)phenyl)carbamoyl)-3,3-dimethylazetidine-2-carboxamido)-[1,1′-biphenyl]-4-carboxylate (120 mg, 0.200 mmol) in methanol (2.0 mL) and water (2.0 mL) at room temperature was added lithium hydroxide monohydrate (173 mg, 4.12 mmol), and the resultant mixture was stirred for 2 hours. Upon completion, the mixture was concentrated under reduced pressure. The crude residue was directly purified by reversed-phase flash chromatography (conditions: column: C18 silica gel; mobile phase A: water (0.1% formic acid); mobile phase B: acetonitrile; gradient (B %): 0%-100% over 25 min; detector: 254 nm) to afford 4′-(1-((3-fluoro-4-(1-(trifluoromethyl)cyclopropyl)phenyl)carbamoyl)-3,3-dimethylazetidine-2-carboxamido)-[1,1′-biphenyl]-4-carboxylic acid (100 mg, 85%) as a white solid. LCMS (ESI) [M+H]⁺: 570.2

### Step 4: (S)-4′-(1-((3-fluoro-4-(1-(trifluoromethyl)cyclopropyl)phenyl)carbamoyl)-3,3-dimethylazetidine-2-carboxamido)-[1,1′-biphenyl]-4-carboxylic acid and (R)-4′-(1-((3-fluoro-4-(1-(trifluoromethyl)cyclopropyl)phenyl)carbamoyl)-3,3-dimethylazetidine-2-carboxamido)-[1,1′-biphenyl]-4-carboxylic acid

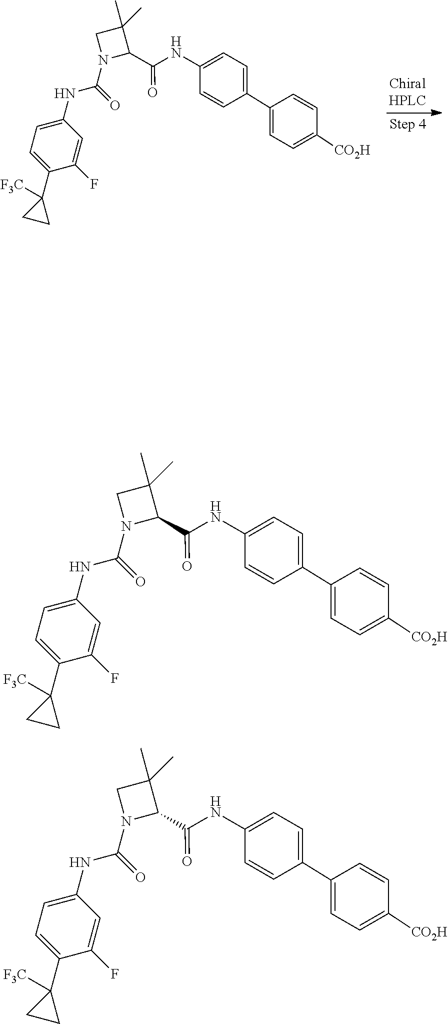 [SMILES: CC1(C)CN(C(=O)Nc2ccc(C3(C(F)(F)F)CC3)c(F)c2)C1C(=O)Nc1ccc(-c2ccc(C(=O)O)cc2)cc1 ; CC1(C)CN(C(=O)Nc2ccc(C3(C(F)(F)F)CC3)c(F)c2)[C@@H]1C(=O)Nc1ccc(-c2ccc(C(=O)O)cc2)cc1 ; CC1(C)CN(C(=O)Nc2ccc(C3(C(F)(F)F)CC3)c(F)c2)[C@H]1C(=O)Nc1ccc(-c2ccc(C(=O)O)cc2)cc1]

4′-(1-((3-fluoro-4-(1-(trifluoromethyl)cyclopropyl)phenyl)carbamoyl)-3,3-dimethylazetidine-2-carboxamido)-[1,1′-biphenyl]-4-carboxylic acid (100 mg) was resolved by preparative chiral HPLC (conditions: column: CHIRAL ART Cellulose-SB, 3*25 cm, 5 μm; mobile phase A: hexane (0.1% formic acid); mobile phase B: ethanol; flow rate: 40 mL/min; gradient (B %) isocratic, 30% B; detector: 220/254 nm; RT1 (min): 7.0; RT2 (min): 11.0) to afford (Compound 292a; Method BQ, Peak 1, 0.69 min) (S)-4′-(1-((3-fluoro-4-(1-(trifluoromethyl)cyclopropyl)phenyl)carbamoyl)-3,3-dimethylazetidine-2-carboxamido)-[1,1′-biphenyl]-4-carboxylic acid (1^{st} peak, 36.6 mg, 36%) as a white solid, and (Compound 292b; Method BQ, Peak 2, 1.08 min) (R)-4′-(1-((3-fluoro-4-(1-(trifluoromethyl)cyclopropyl)phenyl)carbamoyl)-3,3-dimethylazetidine-2-carboxamido)-[1,1′-biphenyl]-4-carboxylic acid (2^{nd} peak, 38.4 mg, 38%) as a white solid.

| Cmpd # | Characterization Data |
|---|---|
| 292a | LCMS (ESI) [M − H]⁻: 568.2 |
| | ¹H NMR (400 MHz, CD₃OD) δ 8.11-8.05 (m, 2H), 7.77-7.70 (m, 4H), 7.70-7.65 (m, 2H), |
| | 7.43 (dd, J = 12.8, 2.2 Hz, 1H), 7.34 (t, J = 8.3 Hz, 1H), 7.24 (dd, J = 8.5, 2.2 Hz, 1H), 4.51 (s, |
| | 1H), 3.87 (d, J = 7.4 Hz, 1H), 3.80 (d, J = 7.4 Hz, 1H), 1.48 (s, 3H), 1.39-1.32 (m, 2H), 1.26 (s, |
| | 3H), 1.04 (s, 2H). |
| 292b | LCMS (ESI) [M − H]⁻: 568.2 |
| | ¹H NMR (400 MHz, CD₃OD) δ 8.07 (d, J = 8.4 Hz, 2H), 7.77-7.71 (m, 4H), 7.68 (d, J = 8.8 Hz, |
| | 2H), 7.43 (dd, J = 12.8, 2.2 Hz, 1H), 7.34 (t, J = 8.3 Hz, 1H), 7.24 (dd, J = 8.5, 2.1 Hz, 1H), 4.51 |
| | (s, 1H), 3.87 (d, J = 7.4 Hz, 1H), 3.80 (d, J = 7.4 Hz, 1H), 1.48 (s, 3H), 1.39-1.32 (m, 2H), 1.26 |
| | (s, 3H), 1.04 (s, 2H). |

### Example 293: (S)-5-(4-(1-((3-fluoro-4-(1-(trifluoromethyl)cyclopropyl)phenyl)carbamoyl)-3,3-dimethylazetidine-2-carboxamido)phenyl)-6-methylpicolinic acid and (R)-5-(4-(1-((3-fluoro-4-(1-(trifluoromethyl)cyclopropyl)phenyl)carbamoyl)-3,3-dimethylazetidine-2-carboxamido)phenyl)-6-methylpicolinic acid

### Step 1: methyl 5-(4-(1-((3-fluoro-4-(1-(trifluoromethyl)cyclopropyl)phenyl)carbamoyl)-3,3-dimethylazetidine-2-carboxamido)phenyl)-6-methylpicolinate

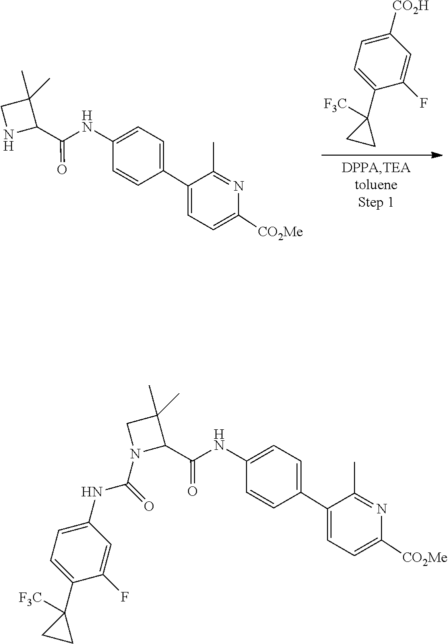 [SMILES: COC(=O)c1ccc(-c2ccc(NC(=O)C3NCC3(C)C)cc2)c(C)n1 ; O=C(O)c1ccc(C2(C(F)(F)F)CC2)c(F)c1 ; COC(=O)c1ccc(-c2ccc(NC(=O)C3N(C(=O)Nc4ccc(C5(C(F)(F)F)CC5)c(F)c4)CC3(C)C)cc2)c(C)n1]

To a solution of 3-fluoro-4-(1-(trifluoromethyl)cyclopropyl)benzoic acid (200 mg, 0.800 mmol) and diphenylphosphoryl azide (444 mg, 1.61 mmol) in toluene (2.0 mL) at room temperature was added sequentially triethylamine (245 mg, 2.41 mmol) and methyl 5-(4-(3,3-dimethylazetidine-2-carboxamido)phenyl)-6-methylpicolinate (285 mg, 0.800 mmol). The resultant mixture was heated to 80° C. and stirred for 2 hours. Upon completion, the reaction mixture was concentrated under reduced pressure. The crude residue was purified by silica gel column chromatography, eluted with petroleum ether/ethyl acetate (1:1) to afford methyl 5-(4-(1-((3-fluoro-4-(1-(trifluoromethyl)cyclopropyl)phenyl)carbamoyl)-3,3-dimethylazetidine-2-carboxamido)phenyl)-6-methylpicolinate (100 mg, 20%) as a pale-yellow oil. LCMS (ESI) [M+H]⁺: 599.2

### Step 2: 5-(4-(1-((3-fluoro-4-(1-(trifluoromethyl)cyclopropyl)phenyl)carbamoyl)-3,3-dimethylazetidine-2-carboxamido)phenyl)-6-methylpicolinic acid

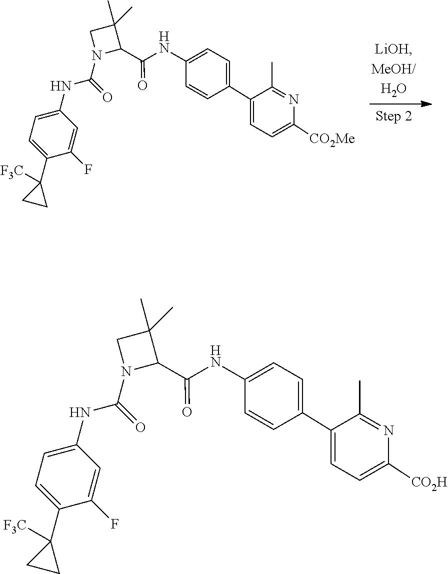 [SMILES: COC(=O)c1ccc(-c2ccc(NC(=O)C3N(C(=O)Nc4ccc(C5(C(F)(F)F)CC5)c(F)c4)CC3(C)C)cc2)c(C)n1 ; Cc1nc(C(=O)O)ccc1-c1ccc(NC(=O)C2N(C(=O)Nc3ccc(C4(C(F)(F)F)CC4)c(F)c3)CC2(C)C)cc1]

To a solution of methyl 5-(4-(1-((3-fluoro-4-(1-(trifluoromethyl)cyclopropyl)phenyl)carbamoyl)-3,3-dimethylazetidine-2-carboxamido)phenyl)-6-methylpicolinate (125 mg, 0.210 mmol) in methanol (2.0 mL) and water (2.0 mL) at room temperature was added lithium hydroxide monohydrate (140 mg, 3.34 mmol), and the resultant mixture was stirred for 2 hours. Upon completion, the mixture was concentrated under reduced pressure. The crude residue was purified by reversed-phase flash chromatography (conditions: column: C18 silica gel; mobile phase A: water (0.1% formic acid); mobile phase B: MeCN; gradient (B %): 0%-100% over 25 min; detector: UV 254 nm) to afford 5-(4-(1-((3-fluoro-4-(1-(trifluoromethyl)cyclopropyl)phenyl)carbamoyl)-3,3-dimethylazetidine-2-carboxamido)phenyl)-6-methylpicolinic acid (70 mg, 56%) as a white solid. LCMS (ESI) [M+H]⁺: 585.2

### Step 3: (S)-5-(4-(1-((3-fluoro-4-(1-(trifluoromethyl)cyclopropyl)phenyl)carbamoyl)-3,3-dimethylazetidine-2-carboxamido)phenyl)-6-methylpicolinic acid and (R)-5-(4-(1-((3-fluoro-4-(1-(trifluoromethyl)cyclopropyl)phenyl)carbamoyl)-3,3-dimethylazetidine-2-carboxamido)phenyl)-6-methylpicolinic acid

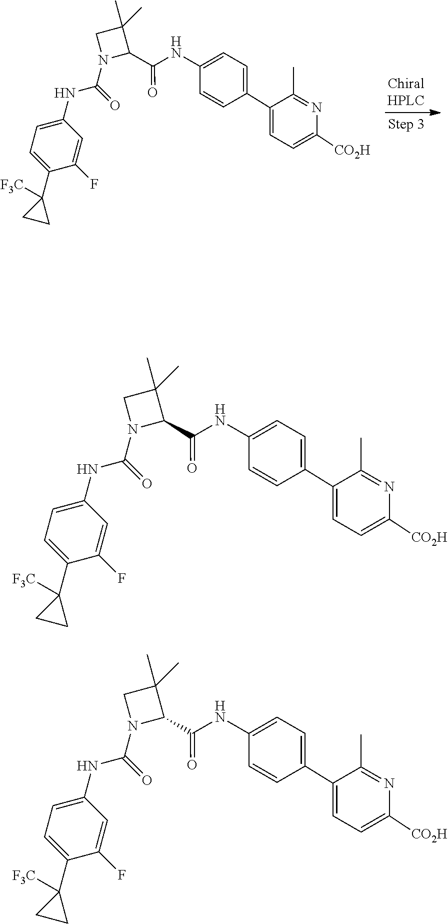 [SMILES: Cc1nc(C(=O)O)ccc1-c1ccc(NC(=O)C2N(C(=O)Nc3ccc(C4(C(F)(F)F)CC4)c(F)c3)CC2(C)C)cc1 ; Cc1nc(C(=O)O)ccc1-c1ccc(NC(=O)[C@H]2N(C(=O)Nc3ccc(C4(C(F)(F)F)CC4)c(F)c3)CC2(C)C)cc1 ; Cc1nc(C(=O)O)ccc1-c1ccc(NC(=O)[C@@H]2N(C(=O)Nc3ccc(C4(C(F)(F)F)CC4)c(F)c3)CC2(C)C)cc1]

5-(4-(1-((3-fluoro-4-(1-(trifluoromethyl)cyclopropyl)phenyl)carbamoyl)-3,3-dimethylazetidine-2-carboxamido)phenyl)-6-methylpicolinic acid (70 mg) was resolved by preparative chiral HPLC (conditions: column: CHIRAL ART Cellulose-SB, 3*25 cm, 5 μm; mobile phase A: hexane (0.1% formic acid), mobile phase B: ethanol; flow rate: 40 mL/min; gradient (B %): isocratic, 50% B; detector: 220/254 nm; RT1 (min): 5.9; RT2 (min): 11.5 to afford (Compound 293a; Method BI, Peak 1, 0.62 min) (S)-5-(4-(1-((3-fluoro-4-(1-(trifluoromethyl)cyclopropyl)phenyl)carbamoyl)-3,3-dimethylazetidine-2-carboxamido)phenyl)-6-methylpicolinic acid (1^{st} peak, 11.2 mg, 16%) as a white solid, and (Compound 293b; Method BI, Peak 2, 1.19 min) (R)-5-(4-(1-((3-fluoro-4-(1-(trifluoromethyl)cyclopropyl)phenyl)carbamoyl)-3,3-dimethylazetidine-2-carboxamido)phenyl)-6-methylpicolinic acid (2^{nd} peak, 22.9 mg, 32%) as a white solid.

| Cmpd # | Characterization Data |
|---|---|
| 293a | LCMS (ESI) [M − H]⁻: 583.2 |
| | ¹H NMR (400 MHz, CD₃OD) δ 8.05 (s, 1H), 7.84 (d, J = 7.8 Hz, 1H), 7.76 (d, J = 8.2 Hz, 2H), |
| | 7.46-7.30 (m, 4H), 7.24 (d, J = 7.6 Hz, 1H), 4.51 (s, 1H), 3.87 (d, J = 7.4 Hz, 1H), 3.80 (d, J = |
| | 7.4 Hz, 1H), 2.56 (s, 3H), 1.48 (s, 3H), 1.40-1.32 (m, 2H), 1.26 (s, 3H), 1.04 (s, 2H). |
| 293b | LCMS (ESI) [M + H]⁺: 585.2 |
| | ¹H NMR (400 MHz, CD₃OD) δ 8.02 (d, J = 7.9 Hz, 1H), 7.82 (d, J = 7.8 Hz, 1H), 7.76 (d, J = |
| | 8.2 Hz, 2H), 7.43 (dd, J = 12.7, 2.1 Hz, 1H), 7.41-7.35 (m, 2H), 7.33 (d, J = 8.3 Hz, 1H), 7.24 |
| | (dd, J = 8.5, 2.1 Hz, 1H), 4.51 (s, 1H), 3.88 (d, J = 7.4 Hz, 1H), 3.80 (d, J = 7.4 Hz, 1H), 2.56 (s, |
| | 3H), 1.48 (s, 3H), 1.38-1.33 (m, 2H), 1.28 (d, J = 8.8 Hz, 3H), 1.04 (s, 2H). |

### Example 294: (S)-4′-(3,3-dimethyl-1-((4-(trifluoromethyl)phenyl)carbamoyl)azetidine-2-carboxamido)-[1,1′-biphenyl]-4-carboxylic acid and (R)-4′-(3,3-dimethyl-1-((4-(trifluoromethyl)phenyl)carbamoyl)azetidine-2-carboxamido)-[1,1′-biphenyl]-4-carboxylic acid

### Step 1: 4′-(3,3-dimethyl-1-((4-(trifluoromethyl)phenyl)carbamoyl)azetidine-2-carboxamido)-[1,1′-biphenyl]-4-carboxylic acid

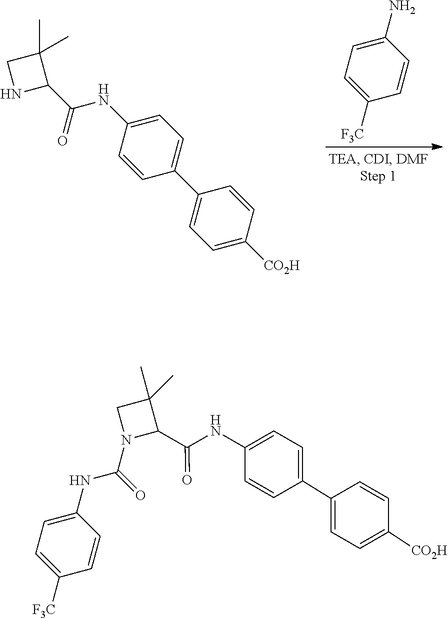 [SMILES: CC1(C)CNC1C(=O)Nc1ccc(-c2ccc(C(=O)O)cc2)cc1 ; Nc1ccc(C(F)(F)F)cc1 ; CC1(C)CN(C(=O)Nc2ccc(C(F)(F)F)cc2)C1C(=O)Nc1ccc(-c2ccc(C(=O)O)cc2)cc1]

A mixture of 4′-(3,3-dimethylazetidine-2-carboxamido)-[1,1′-biphenyl]-4-carboxylic acid (358 mg, 1.19 mmol), para-trifluoromethylaniline (60 mg, 0.372 mmol), triethylamine (113 mg, 1.12 mmol) and carbonyldiimidazole (121 mg, 0.744 mmol) in DMF (1.0 mL) was heated to 50° C. for 1 hour. Upon completion, the mixture was concentrated under reduced pressure. The crude residue was purified by reverse phase flash column chromatography (conditions: column: C18 silica gel; mobile phase A: water (0.1% NH₄HCO₃); mobile phase B: MeCN; gradient (B %): 0%-100% over 25 min; detector: UV 254 nm) to afford 4′-(3,3-dimethyl-1-((4-(trifluoromethyl)phenyl)carbamoyl)azetidine-2-carboxamido)-[1,1′-biphenyl]-4-carboxylic acid (60 mg, 31%) as a brown solid. LCMS (ESI) [M+H]⁺: 512

### Step 2: (S)-4′-(3,3-dimethyl-1-((4-(trifluoromethyl)phenyl)carbamoyl)azetidine-2-carboxamido)-[1,1′-biphenyl]-4-carboxylic acid and (R)-4′-(3,3-dimethyl-1-((4-(trifluoromethyl)phenyl)carbamoyl)azetidine-2-carboxamido)-[1,1′-biphenyl]-4-carboxylic

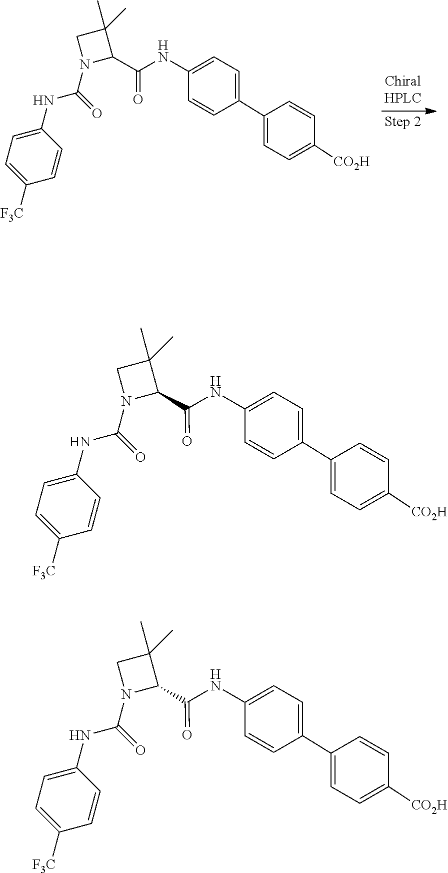 [SMILES: CC1(C)CN(C(=O)Nc2ccc(C(F)(F)F)cc2)C1C(=O)Nc1ccc(-c2ccc(C(=O)O)cc2)cc1 ; CC1(C)CN(C(=O)Nc2ccc(C(F)(F)F)cc2)[C@@H]1C(=O)Nc1ccc(-c2ccc(C(=O)O)cc2)cc1 ; CC1(C)CN(C(=O)Nc2ccc(C(F)(F)F)cc2)[C@H]1C(=O)Nc1ccc(-c2ccc(C(=O)O)cc2)cc1]

4′-(3,3-dimethyl-1-((4-(trifluoromethyl)phenyl)carbamoyl)azetidine-2-carboxamido)-[1,1′-biphenyl]-4-carboxylic acid (60 mg) was resolved by preparative chiral HPLC (conditions: column: CHIRAL ART Cellulose-SZ, 3*25 cm, 5 μm; mobile phase A: MTBE (0.1% formic acid); mobile phase B: methanol; flow rate: 40 mL/min; gradient (B %) isocratic, 30% B; detector: 220/254 nm; RT1 (min): 4.9; RT2 (min): 7.2) to afford (Compound 294a; Method BR, Peak 1, 0.99 min) (S)-4′-(3,3-dimethyl-1-((4-(trifluoromethyl)phenyl)carbamoyl)azetidine-2-carboxamido)-[1,1′-biphenyl]-4-carboxylic acid (1^{st} peak, 25.1 mg, 42%) as a white solid, and (Compound 294b; Method BR, Peak 2, 2.50 min) (R)-4′-(3,3-dimethyl-1-((4-(trifluoromethyl)phenyl)carbamoyl)azetidine-2-carboxamido)-[1,1′-biphenyl]-4-carboxylic acid (2^{nd} peak, 14.5 mg, 24%) as a white solid.

| Cmpd # | Characterization Data |
|---|---|
| 294a | LCMS (ESI) [M + H]⁺: 512.2 |
| | ¹H NMR (400 MHz, DMSO-d₆) δ |
| | 12.92 (s, 1H), 10.05 (s, 1H), 8.95 (s, 1H), 8.00 (d, J = |
| | 8.1 Hz, 2H), 7.84-7.68 (m, 8H), 7.60 (d, J = 8.9 Hz, 2H), |
| | 4.45 (s, 1H), 3.79 (d, J = 7.4 Hz, 1H), 3.71 |
| | (d, J = 7.4 Hz, 1H), 1.42 (s, 3H), 1.12 (s, 3H). |
| 294b | LCMS (ESI) [M + H]⁺: 512.2 |
| | ¹H NMR (400 MHz, DMSO-d₆) δ |
| | 12.94 (s, 1H), 10.06 (s, 1H), 8.95 (s, 1H), 8.01 (d, J = |
| | 8.3 Hz, 2H), 7.84-7.70 (m, 8H), 7.60 (d, J = 8.6 Hz, 2H), |
| | 4.46 (s, 1H), 3.80 (d, J = 7.4 Hz, 1H), 3.71 |
| | (d, J = 7.5 Hz, 1H), 1.43 (s, 3H), 1.13 (s, 3H). |

### Example 295: (S)-5-(4-(3,3-dimethyl-1-((4-(1-(trifluoromethyl)cyclopropyl)phenyl)carbamoyl)azetidine-2-carboxamido)phenyl)-6-methylpicolinic acid and (R)-5-(4-(3,3-dimethyl-1-((4-(1-(trifluoromethyl)cyclopropyl)phenyl)carbamoyl)azetidine-2-carboxamido)phenyl)-6-methylpicolinic acid

### Step 1: 5-(4-(3,3-dimethyl-1-((4-(1-(trifluoromethyl)cyclopropyl)phenyl)carbamoyl)azetidine-2-carboxamido)phenyl)-6-methylpicolinic acid

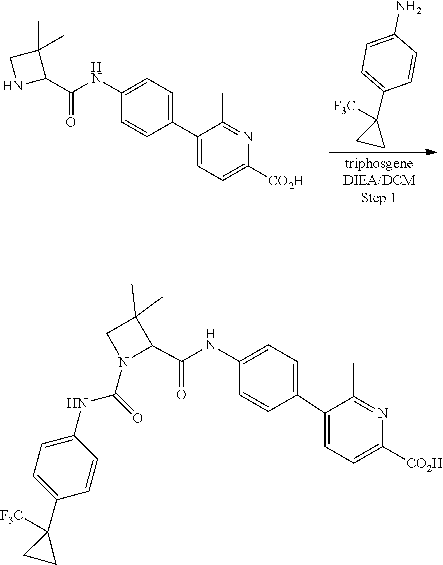 [SMILES: Cc1nc(C(=O)O)ccc1-c1ccc(NC(=O)C2NCC2(C)C)cc1 ; Nc1ccc(C2(C(F)(F)F)CC2)cc1 ; Cc1nc(C(=O)O)ccc1-c1ccc(NC(=O)C2N(C(=O)Nc3ccc(C4(C(F)(F)F)CC4)cc3)CC2(C)C)cc1]

A solution of 4-[1-(trifluoromethyl)cyclopropyl]aniline (50.0 mg, 0.249 mmol) and 5-(4-(3,3-dimethylazetidine-2-carboxamido)phenyl)-6-methylpicolinic acid (84.0 mg, 0.249 mmol), N,N-Diisopropylethylamine (160 mg, 1.25 mmol) and triphosgene (36.0 mg, 0.124 mmol) in dichloromethane (2.0 mL) was stirred at 25° C. for 1 hour. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0%-100% over 30 min; detector: UV 254 nm) to afford 5-(4-(3,3-dimethyl-1-((4-(1-(trifluoromethyl)cyclopropyl)phenyl)carbamoyl)azetidine-2-carboxamido)phenyl)-6-methylpicolinic acid (65 mg, 46%) as a white solid. LCMS (ESI) [M+H]⁺: 567.2

### Step 2: (S)-5-(4-(3,3-dimethyl-1-((4-(1-(trifluoromethyl)cyclopropyl)phenyl)carbamoyl)azetidine-2-carboxamido)phenyl)-6-methylpicolinic acid and (R)-5-(4-(3,3-dimethyl-1-((4-(1-(trifluoromethyl)cyclopropyl)phenyl)carbamoyl)azetidine-2-carboxamido)phenyl)-6-methylpicolinic acid

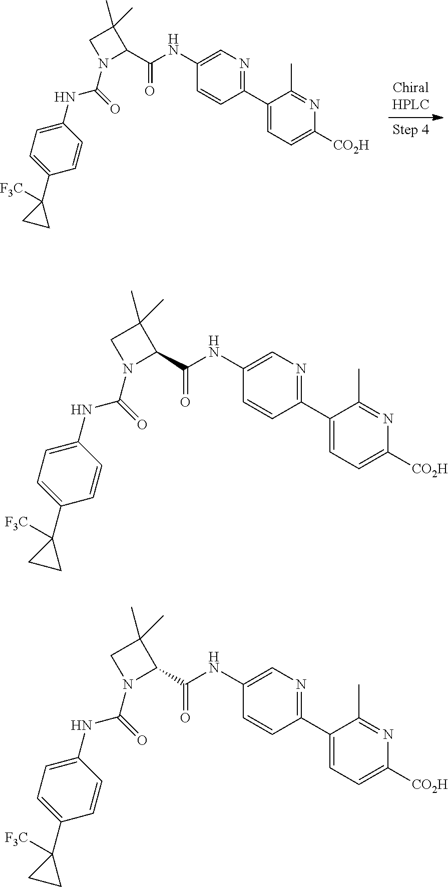 [SMILES: Cc1nc(C(=O)O)ccc1-c1ccc(NC(=O)C2N(C(=O)Nc3ccc(C4(C(F)(F)F)CC4)cc3)CC2(C)C)cn1 ; Cc1nc(C(=O)O)ccc1-c1ccc(NC(=O)[C@H]2N(C(=O)Nc3ccc(C4(C(F)(F)F)CC4)cc3)CC2(C)C)cn1 ; Cc1nc(C(=O)O)ccc1-c1ccc(NC(=O)[C@@H]2N(C(=O)Nc3ccc(C4(C(F)(F)F)CC4)cc3)CC2(C)C)cn1]

5-(4-(3,3-dimethyl-1-((4-(1-(trifluoromethyl)cyclopropyl)phenyl)carbamoyl)azetidine-2-carboxamido)phenyl)-6-methylpicolinic acid (65 mg) was resolved by preparative chiral HPLC (conditions: column: CHIRAL ART Cellulose-SZ, 3*25 cm, 5 μm; mobile phase A: MTBE (0.1% formic acid), mobile phase B: methanol; flow rate: 40 mL/min; gradient (B %): isocratic, 50% B; detector: 220/254 nm; RT1 (min): 7.1; RT2 (min): 17.9) to afford (Compound 295a; Method BL, Peak 1, 0.83 min) (S)-5-(4-(3,3-dimethyl-1-((4-(1-(trifluoromethyl)cyclopropyl)phenyl)carbamoyl)azetidine-2-carboxamido)phenyl)-6-methylpicolinic acid (1^{st} peak, 28.3 mg, 47%) as a white solid, and (Compound 295b; Method BL, Peak 2, 2.71 min) (R)-5-(4-(3,3-dimethyl-1-((4-(1-(trifluoromethyl)cyclopropyl)phenyl)carbamoyl)azetidine-2-carboxamido)phenyl)-6-methylpicolinic acid (2^{nd} peak, 27.7 mg, 46%) as a white solid.

| Cmpd # | Characterization Data |
|---|---|
| 295a | LCMS (ESI) [M + H]⁺: 567.3 |
| | ¹H NMR (400 MHz, CD₃OD) δ 8.08 |
| | (d, J = 7.9 Hz, 1H), 7.89 (d, J = 7.8 Hz, 1H), 7.77 (d, J = |
| | 8.4 Hz, 2H), 7.50-7.44 (m, 2H), 7.38 (dd, J = 11.4, 8.4 Hz, 4H), |
| | 4.51 (s, 1H), 3.87 (d, J = 7.3 Hz, 1H), 3.80 (d, J = 7.3 Hz, 1H), |
| | 2.59 (s, 3H), 1.48 (s, 3H), 1.35-1.20 (m, 5H), 1.02 (s, 2H). |
| 295b | LCMS (ESI) [M + H]⁺: 567.3 |
| | ¹H NMR (400 MHz, CD₃OD) δ 8.07 |
| | (d, J = 7.9 Hz, 1H), 7.86 (d, J = 7.8 Hz, 1H), 7.77 (d, J = |
| | 8.3 Hz, 2H), 7.47 (d, J = 8.7 Hz, 2H), 7.38 (t, J = 9.1 Hz, 4H), |
| | 4.51 (s, 1H), 3.87 (d, J = 7.3 Hz, 1H), 3.80 (d, J = 7.4 Hz, |
| | 1H), 2.58 (s, 3H), 1.48 (s, 3H), 1.35-1.24 (m, 5H), |
| | 1.06-1.00 (m, 2H). |

### Example 296: (S)-4′-(3,3-dimethyl-1-((4-(1-(trifluoromethyl)cyclopropyl)phenyl)carbamoyl)azetidine-2-carboxamido)-[1,1′-biphenyl]-4-carboxylic acid and (R)-4′-(3,3-dimethyl-1-((4-(1-(trifluoromethyl)cyclopropyl)phenyl)carbamoyl)azetidine-2-carboxamido)-[1,1′-biphenyl]-4-carboxylic acid

### Step 1: 4′-(3,3-dimethyl-1-((4-(1-(trifluoromethyl)cyclopropyl)phenyl)carbamoyl)azetidine-2-carboxamido)-[1,1′-biphenyl]-4-carboxylic acid

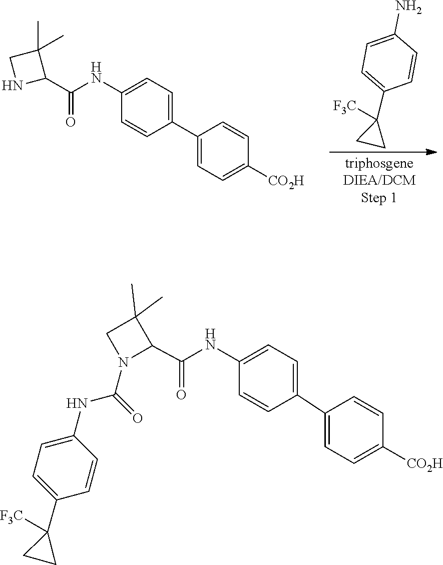 [SMILES: CC1(C)CNC1C(=O)Nc1ccc(-c2ccc(C(=O)O)cc2)cc1 ; Nc1ccc(C2(C(F)(F)F)CC2)cc1 ; CC1(C)CN(C(=O)Nc2ccc(C3(C(F)(F)F)CC3)cc2)C1C(=O)Nc1ccc(-c2ccc(C(=O)O)cc2)cc1]

A solution of 4-(1-(trifluoromethyl)cyclopropyl)aniline (50.0 mg, 0.249 mmol), 4′-(3,3-dimethylazetidine-2-carboxamido)-[1,1′-biphenyl]-4-carboxylic acid (84.0 mg, 0.249 mmol), N,N-Diisopropylethylamine (160 mg, 1.25 mmol), and triphosgene (36.0 mg, 0.124 mmol) in dichloromethane (2.0 mL) was stirred at 25° C. for 1 hour. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0%-100% over 30 min; detector: UV 254 nm) to afford 4′-(3,3-dimethyl-1-((4-(1-(trifluoromethyl)cyclopropyl)phenyl)carbamoyl)azetidine-2-carboxamido)-[1,1′-biphenyl]-4-carboxylic acid (102 mg, 66%) as a white solid. LCMS (ESI) [M+H]⁺: 552.2

### Step 2: (S)-4′-(3,3-dimethyl-1-((4-(1-(trifluoromethyl)cyclopropyl)phenyl)carbamoyl)azetidine-2-carboxamido)-[1,1′-biphenyl]-4-carboxylic acid and (R)-4′-(3,3-dimethyl-1-((4-(1-(trifluoromethyl)cyclopropyl)phenyl)carbamoyl)azetidine-2-carboxamido)-[1,1′-biphenyl]-4-carboxylic acid

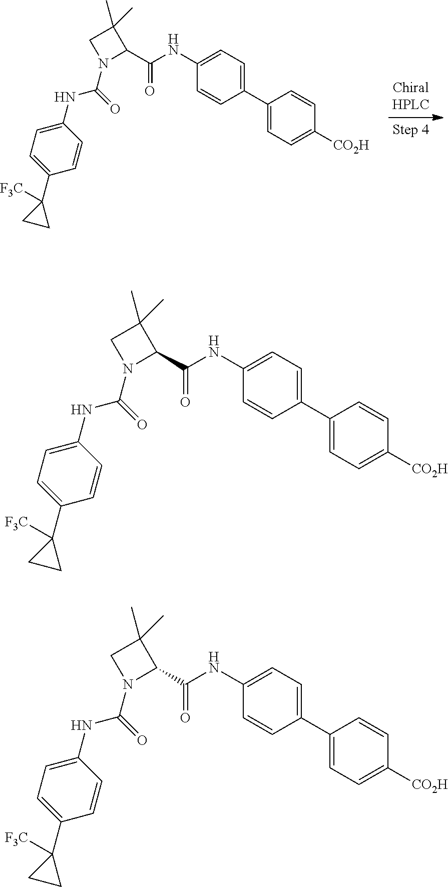 [SMILES: CC1(C)CN(C(=O)Nc2ccc(C3(C(F)(F)F)CC3)cc2)C1C(=O)Nc1ccc(-c2ccc(C(=O)O)cc2)cc1 ; CC1(C)CN(C(=O)Nc2ccc(C3(C(F)(F)F)CC3)cc2)[C@@H]1C(=O)Nc1ccc(-c2ccc(C(=O)O)cc2)cc1 ; CC1(C)CN(C(=O)Nc2ccc(C3(C(F)(F)F)CC3)cc2)[C@H]1C(=O)Nc1ccc(-c2ccc(C(=O)O)cc2)cc1]

4′-(3,3-dimethyl-1-((4-(1-(trifluoromethyl)cyclopropyl)phenyl)carbamoyl)azetidine-2-carboxamido)-[1,1′-biphenyl]-4-carboxylic acid (100 mg) was resolved by preparative chiral HPLC (conditions: column: CHIRAL ART Cellulose-SB, 3*25 cm, 5 μm; mobile phase A: hexane (0.1% formic acid); mobile phase B: ethanol; flow rate: 40 mL/min; gradient (B %) isocratic, 30% B; detector: 220/254 nm; RT1 (min): 7.8; RT2 (min): 15.1) to afford (Compound 296a; Method BS, Peak 1, 1.34 min) (S)-4′-(3,3-dimethyl-1-((4-(1-(trifluoromethyl)cyclopropyl)phenyl)carbamoyl)azetidine-2-carboxamido)-[1,1′-biphenyl]-4-carboxylic acid (1^{st} peak, 30.8 mg, 47%) as a white solid, and (Compound 296b; Method BS, Peak 2, 2.52 min) (R)-4′-(3,3-dimethyl-1-((4-(1-(trifluoromethyl)cyclopropyl)phenyl)carbamoyl)azetidine-2-carboxamido)-[1,1′-biphenyl]-4-carboxylic acid (2^{nd} peak, 30.7 mg, 47%) as a white solid.

| Cmpd # | Characterization Data |
|---|---|
| 296a | LCMS (ESI) [M + H]⁺: 552.3 |
| | ¹H NMR (400 MHz, CD₃OD) δ |
| | 8.08 (d, J = 8.5 Hz, 2H), 7.77-7.70 (m, 4H), 7.67 (d, J = |
| | 8.8 Hz, 2H), 7.47 (d, J = 8.6 Hz, 2H), 7.37 (d, J = 8.6 Hz, 2H), |
| | 4.50 (s, 1H), 3.87 (d, J = 7.4 Hz, 1H), 3.79 (d, J = 7.4 Hz, 1H), |
| | 1.48 (s, 3H), 1.35-1.22 (m, 5H), 1.07-0.93 (m, 2H). |
| 296b | LCMS (ESI) [M + H]⁺: 552.3 |
| | ¹H NMR (400 MHz, CD₃OD) δ 8.07 |
| | (d, J = 8.4 Hz, 2H), 7.77-7.70 (m, 4H), 7.67 (d, J = 8.8 Hz, |
| | 2H), 7.47 (d, J = 8.6 Hz, 2H), 7.36 (d, J = 8.6 Hz, 2H), |
| | 4.50 (s, 1H), 3.87 (d, J = 7.4 Hz, 1H), 3.79 |
| | (d, J = 7.4 Hz, 1H), 1.47 (s, 3H), 1.35-1.23 (m, 5H), |
| | 1.04-0.98 (m, 2H). |

### Example 297: (S)-4′-(1-((3-fluoro-4-(trifluoromethyl)phenyl)carbamoyl)-3,3-dimethylazetidine-2-carboxamido)-[1,1′-biphenyl]-4-carboxylic acid and (R)-4′-(1-((3-fluoro-4-(trifluoromethyl)phenyl)carbamoyl)-3,3-dimethylazetidine-2-carboxamido)-[1,1′-biphenyl]-4-carboxylic acid

### Step 1: 4′-(1-((3-fluoro-4-(trifluoromethyl)phenyl)carbamoyl)-3,3-dimethylazetidine-2-carboxamido)-[1,1′-biphenyl]-4-carboxylic acid

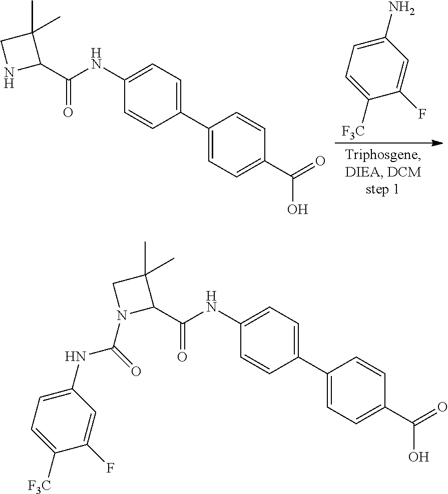 [SMILES: CC1(C)CNC1C(=O)Nc1ccc(-c2ccc(C(=O)O)cc2)cc1 ; Nc1ccc(C(F)(F)F)c(F)c1 ; CC1(C)CN(C(=O)Nc2ccc(C(F)(F)F)c(F)c2)C1C(=O)Nc1ccc(-c2ccc(C(=O)O)cc2)cc1]

A solution of 3-fluoro-4-(trifluoromethyl)aniline (82.8 mg, 0.462 mmol), 4′-(3,3-dimethylazetidine-2-carboxamido)-[1,1′-biphenyl]-4-carboxylic acid (100 mg, 0.308 mmol), N,N-Diisopropylethylamine (160 mg, 1.245 mmol) and triphosgene (45.7 mg, 0.154 mmol) in dichloromethane (2.0 mL) was stirred at 25° C. for 1 hour. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0%-100% over 30 min; detector: 254 nm) to afford 4′-(1-((3-fluoro-4-(trifluoromethyl)phenyl)carbamoyl)-3,3-dimethylazetidine-2-carboxamido)-[1,1′-biphenyl]-4-carboxylic acid (85.0 mg, 52%) as a white solid. LCMS (ESI) [M+H]⁺: 530.5

### Step 2: (S)-4′-(1-((3-fluoro-4-(trifluoromethyl)phenyl)carbamoyl)-3,3-dimethylazetidine-2-carboxamido)-[1,1′-biphenyl]-4-carboxylic acid and (R)-4′-(1-((3-fluoro-4-(trifluoromethyl)phenyl)carbamoyl)-3,3-dimethylazetidine-2-carboxamido)-[1,1′-biphenyl]-4-carboxylic acid

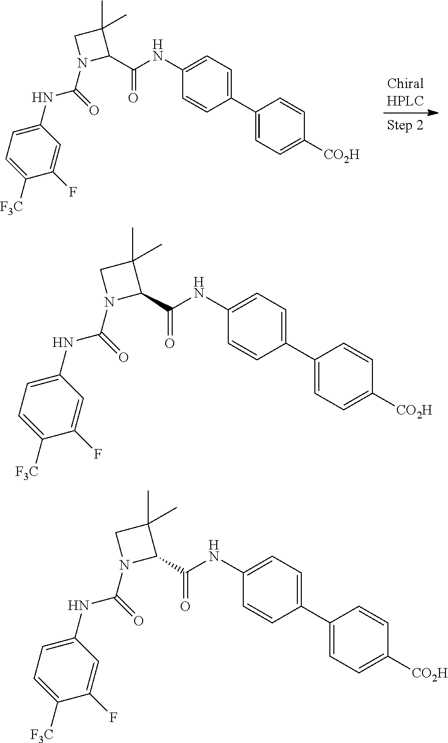 [SMILES: CC1(C)CN(C(=O)Nc2ccc(C(F)(F)F)c(F)c2)C1C(=O)Nc1ccc(-c2ccc(C(=O)O)cc2)cc1 ; CC1(C)CN(C(=O)Nc2ccc(C(F)(F)F)c(F)c2)[C@@H]1C(=O)Nc1ccc(-c2ccc(C(=O)O)cc2)cc1 ; CC1(C)CN(C(=O)Nc2ccc(C(F)(F)F)c(F)c2)[C@H]1C(=O)Nc1ccc(-c2ccc(C(=O)O)cc2)cc1]

4′-(1-((3-fluoro-4-(trifluoromethyl)phenyl)carbamoyl)-3,3-dimethylazetidine-2-carboxamido)-[1,1′-biphenyl]-4-carboxylic acid (80 mg) was resolved by preparative chiral HPLC (conditions: column: CHIRAL ART Cellulose-SB, 3*25 cm, 5 μm; mobile phase A: hexane (0.1% formic acid); mobile phase B: ethanol; flow rate: 40 mL/min; gradient (B %) isocratic, 30% B; detector: 220/254 nm; RT1 (min): 7.8; RT2 (min): 15.1) to afford (Compound 297a; Method BQ, Peak 1, 0.62 min) (S)-4′-(1-((3-fluoro-4-(trifluoromethyl)phenyl)carbamoyl)-3,3-dimethylazetidine-2-carboxamido)-[1,1′-biphenyl]-4-carboxylic acid (1^{st} peak, 31.9 mg, 40%) as a white solid and (Compound 297b; Method BQ, Peak 2, 1.01 min) (R)-4′-(1-((3-fluoro-4-(trifluoromethyl)phenyl)carbamoyl)-3,3-dimethylazetidine-2-carboxamido)-[1,1′-biphenyl]-4-carboxylic acid (2^{nd} peak, 24.7 mg, 31%) as a white solid.

| Cmpd # | Characterization Data |
|---|---|
| 297a | LCMS (ESI) [M + H]⁺: 530.2 |
| | ¹H NMR (400 MHz, CD₃OD) δ 8.07 |
| | (d, J = 8.1 Hz, 2H), 7.78-7.63 (m, 7H), 7.52 (t, J = 8.4 Hz, |
| | 1H), 7.38 (dd, J = 8.7, 2.0 Hz, 1H), 4.54 (s, 1H), 3.90 (d, J = |
| | 7.5 Hz, 1H), 3.82 (d, J = 7.4 Hz, 1H), 1.49 (s, 3H), 1.26 (s, 3H). |
| 297b | LCMS (ESI) [M + H]⁺: 530.2 |
| | ¹H NMR (400 MHz, CD₃OD) δ |
| | 8.07 (d, J = 8.1 Hz, 2H), 7.78-7.63 (m, 7H), 7.52 |
| | (t, J = 8.4 Hz, 1H), 7.38 (dd, J = 8.7, 2.0 Hz, 1H), |
| | 4.54 (s, 1H), 3.90 (d, J = 7.4 Hz, 1H), 3.82 (d, J = |
| | 7.3 Hz, 1H), 1.49 (s, 3H), 1.26 (s, 3H). |

### Example 298: (R)-4-(2-(1-((3-fluoro-4-(trifluoromethyl)phenyl)carbamoyl)pyrrolidin-2-yl)benzo[d]oxazol-6-yl)benzoic acid and (S)-4-(2-(1-((3-fluoro-4-(trifluoromethyl)phenyl)carbamoyl)pyrrolidin-2-yl)benzo[d]oxazol-6-yl)benzoic acid

### Step 1: tert-butyl 2-(6-(4-(tert-butoxycarbonyl)phenyl)benzo[d]oxazol-2-yl)pyrrolidine-1-carboxylate

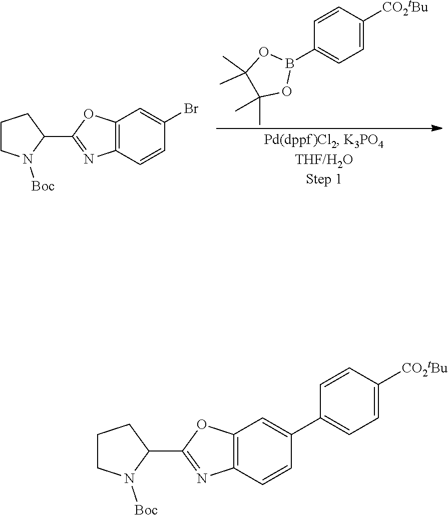

A solution of tert-butyl 2-(6-bromobenzo[d]oxazol-2-yl)pyrrolidine-1-carboxylate (500 mg, 1.36 mmol), tert-butyl 4-(4,4,5,5-tetramethyl-1,3,2-dioxaborolan-2-yl)benzoate (414 mg, 1.36 mmol), K₃PO₄ (866 mg, 4.08 mmol) and Pd(dppf)Cl₂ (99 mg, 0.136 mmol) in THF (3.0 mL) and water (0.30 mL) was heated at 80° C. for 2 hours. Upon completion, the reaction mixture was cooled to ambient temperature and concentrated under reduced pressure. The crude residue was directly purified by silica gel column chromatography, eluted with petroleum ether/ethyl acetate (1:1), to afford tert-butyl 2-(6-(4-(tert-butoxycarbonyl)phenyl)benzo[d]oxazol-2-yl)pyrrolidine-1-carboxylate (300 mg, 47%) as a white solid. LCMS (ESI) [M+H]⁺: 465.2

### Step 2: 4-(2-(pyrrolidin-2-yl)benzo[d]oxazol-6-yl)benzoic acid

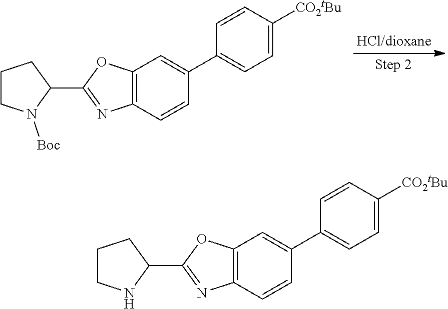 [SMILES: CC(C)(C)OC(=O)c1ccc(-c2ccc3nc(C4CCCN4C(=O)OC(C)(C)C)oc3c2)cc1 ; CC(C)(C)OC(=O)c1ccc(-c2ccc3nc(C4CCCN4)oc3c2)cc1]

To a solution of tert-butyl 2-(6-(4-(tert-butoxycarbonyl)phenyl)benzo[d]oxazol-2-yl)pyrrolidine-1-carboxylate (300 mg, 0.646 mmol) in 1,4-dioxane (3.0 mL) at room temperature was added a solution of hydrochloric acid (3.0 mL, 12 mmol, 4.0 M in 1,4-dioxane). After 2 hours, the reaction mixture was concentrated under reduced pressure to afford 4-(2-(pyrrolidin-2-yl)benzo[d]oxazol-6-yl)benzoic acid (160 mg, crude) as a white solid, which was used directly in the next step without further purification. LCMS (ESI) [M+H]⁺: 309.1

### Step 3: 4-(2-(1-((3-fluoro-4-(trifluoromethyl)phenyl)carbamoyl)pyrrolidin-2-yl)benzo[d]oxazol-6-yl)benzoic acid

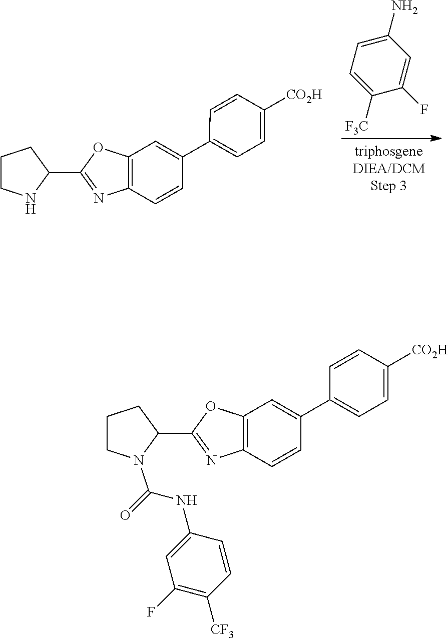 [SMILES: O=C(O)c1ccc(-c2ccc3nc(C4CCCN4)oc3c2)cc1 ; Nc1ccc(C(F)(F)F)c(F)c1 ; O=C(O)c1ccc(-c2ccc3nc(C4CCCN4C(=O)Nc4ccc(C(F)(F)F)c(F)c4)oc3c2)cc1]

A solution of 3-fluoro-4-(trifluoromethyl)aniline (92 mg, 0.519 mmol), DIEA (335 mg, 2.60 mmol), 4-(2-(pyrrolidin-2-yl)benzo[d]oxazol-6-yl)benzoic acid (160 mg, 0.519 mmol) and triphosgene (76.0 mg, 0.260 mmol) in dichloromethane (2.0 mL) was stirred at room temperature for 1 hour. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0%-100% over 30 min; detector: 254 nm) to afford 4-(2-(1-((3-fluoro-4-(trifluoromethyl)phenyl)carbamoyl)pyrrolidin-2-yl)benzo[d]oxazol-6-yl)benzoic acid (140 mg, 52%) as a white solid LCMS (ESI) [M+H]⁺: 514.1

### Step 4: (R)-4-(2-(1-((3-fluoro-4-(trifluoromethyl)phenyl)carbamoyl)pyrrolidin-2-yl)benzo[d]oxazol-6-yl)benzoic acid and (S)-4-(2-(1-((3-fluoro-4-(trifluoromethyl)phenyl)carbamoyl)pyrrolidin-2-yl)benzo[d]oxazol-6-yl)benzoic acid

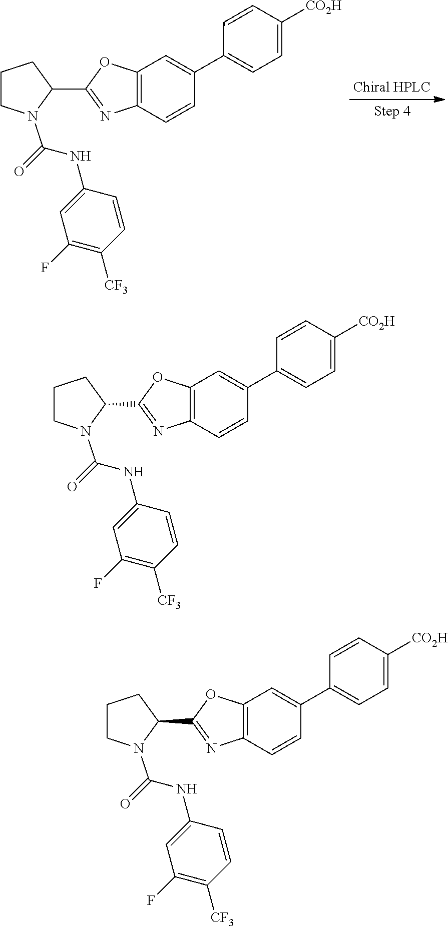 [SMILES: O=C(O)c1ccc(-c2ccc3nc(C4CCCN4C(=O)Nc4ccc(C(F)(F)F)c(F)c4)oc3c2)cc1 ; O=C(O)c1ccc(-c2ccc3nc([C@H]4CCCN4C(=O)Nc4ccc(C(F)(F)F)c(F)c4)oc3c2)cc1 ; O=C(O)c1ccc(-c2ccc3nc([C@@H]4CCCN4C(=O)Nc4ccc(C(F)(F)F)c(F)c4)oc3c2)cc1]

4-(2-(1-((3-fluoro-4-(trifluoromethyl)phenyl)carbamoyl)pyrrolidin-2-yl)benzo[d]oxazol-6-yl)benzoic acid (140 mg) was resolved by preparative chiral HPLC (conditions: column: CHIRALPAK IG, 3*25 cm, 5 μm; mobile phase A: hexane (0.1% formic acid); mobile phase B: 2-propanol; flow rate: 40 mL/min; gradient (B %) isocratic, 50% B; detector: 220/254 nm; RT1 (min): 7.6; RT2 (min): 13.9) to afford (Compound 298a; Method BT, Peak 1, 0.85 min) (R)-4-(2-(1-((3-fluoro-4-(trifluoromethyl)phenyl)carbamoyl)pyrrolidin-2-yl)benzo[d]oxazol-6-yl)benzoic acid (1^{st} peak, 122.4 mg, 87%) as a white solid, and (Compound 298b; Method BT, Peak 2, 1.44 min) (S)-4-(2-(1-((3-fluoro-4-(trifluoromethyl)phenyl)carbamoyl)pyrrolidin-2-yl)benzo[d]oxazol-6-yl)benzoic acid (2^{nd} peak, 10.6 mg, 7%) as a white solid.

| Cmpd # | Characterization Data |
|---|---|
| 298a | LCMS (ESI) [M + H]⁺: 514.2 |
| | ¹H NMR (400 MHz, CD₃OD) δ 8.11 |
| | (d, J = 8.4 Hz, 2H), 7.95 (s, 1H), 7.79 (d, J = 8.4 Hz, |
| | 2H), 7.77-7.69 (m, 2H), 7.64-7.55 (m, 1H), 7.51 (t, J = |
| | 8.4 Hz, 1H), 7.38 (d, J = 8.8 Hz, 1H), 5.37 (dd, J = 8.0, |
| | 2.7 Hz, 1H), 3.95-3.81 (m, 1H), 3.78-3.65 (m, 1H), 2.55-2.43 |
| | (m, 1H), 2.32-2.15 (m, 3H). |
| 298b | LCMS (ESI) [M + H]⁺: 514.2 |
| | ¹H NMR (400 MHz, CD₃OD) δ 8.11 |
| | (d, J = 8.3 Hz, 2H), 7.94 (s, 1H), 7.78 (d, J = 8.2 Hz, 2H), |
| | 7.76-7.69 (m, 2H), 7.59 (dd, J = 13.7, 2.0 Hz, 1H), |
| | 7.50 (t, J = 8.5 Hz, 1H), 7.41-7.35 (m, 1H), 5.37 (dd, J = |
| | 8.2, 2.7 Hz, 1H), 3.95-3.82 (m, 1H), 3.76-3.65 (m, 1H), |
| | 2.55-2.43 (m, 1H), 2.33-2.15 (m, 3H). |

### Example 299: (S)-4′-(1-((2,2-difluorobenzo[d][1,3]dioxol-5-yl)carbamoyl)-3,3-dimethylazetidine-2-carboxamido)-[1,1′-biphenyl]-4-carboxylic acid and (R)-4′-(1-((2,2-difluorobenzo[d][1,3]dioxol-5-yl)carbamoyl)-3,3-dimethylazetidine-2-carboxamido)-[1,1′-biphenyl]-4-carboxylic acid

### Step 1: 4′-(1-((2,2-difluorobenzo[d][1,3]dioxol-5-yl)carbamoyl)-3,3-dimethylazetidine-2-carboxamido)-[1,1′-biphenyl]-4-carboxylic acid

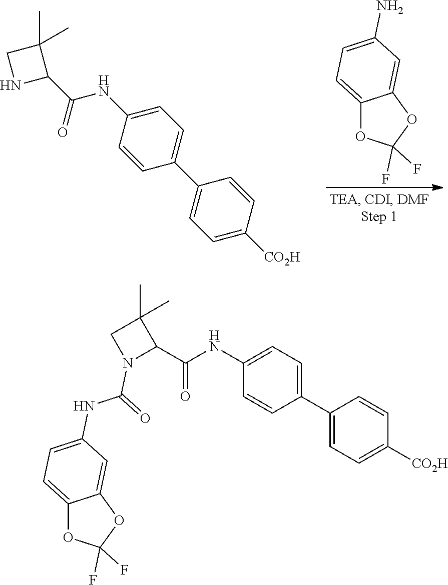 [SMILES: CC1(C)CNC1C(=O)Nc1ccc(-c2ccc(C(=O)O)cc2)cc1 ; Nc1ccc2c(c1)OC(F)(F)O2 ; CC1(C)CN(C(=O)Nc2ccc3c(c2)OC(F)(F)O3)C1C(=O)Nc1ccc(-c2ccc(C(=O)O)cc2)cc1]

A solution of 2,2-difluoro-1,3-benzodioxol-5-amine (300 mg, 1.73 mmol), carbonyldiimidazole (1.12 g, 6.93 mmol), 4′-(3,3-dimethylazetidine-2-carboxamido)-[1,1′-biphenyl]-4-carboxylic acid (562 mg, 1.73 mmol) and triethylamine (701 mg, 6.93 mmol) in DMF (3.0 mL) was stirred at 40° C. for 2 hours. Upon completion, the mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0%-100% over 30 min; detector: 254 nm) to afford 4′-(1-((2,2-difluorobenzo[d][1,3]dioxol-5-yl)carbamoyl)-3,3-dimethylazetidine-2-carboxamido)-[1,1′-biphenyl]-4-carboxylic acid (100 mg, 11%) as a white solid. LCMS (ESI) [M+H]⁺: 524.1

### Step 2: (S)-4′-(1-((2,2-difluorobenzo[d][1,3]dioxol-5-yl)carbamoyl)-3,3-dimethylazetidine-2-carboxamido)-[1,1′-biphenyl]-4-carboxylic acid and (R)-4′-(1-((2,2-difluorobenzo[d][1,3]dioxol-5-yl)carbamoyl)-3,3-dimethylazetidine-2-carboxamido)-[1,1′-biphenyl]-4-carboxylic acid

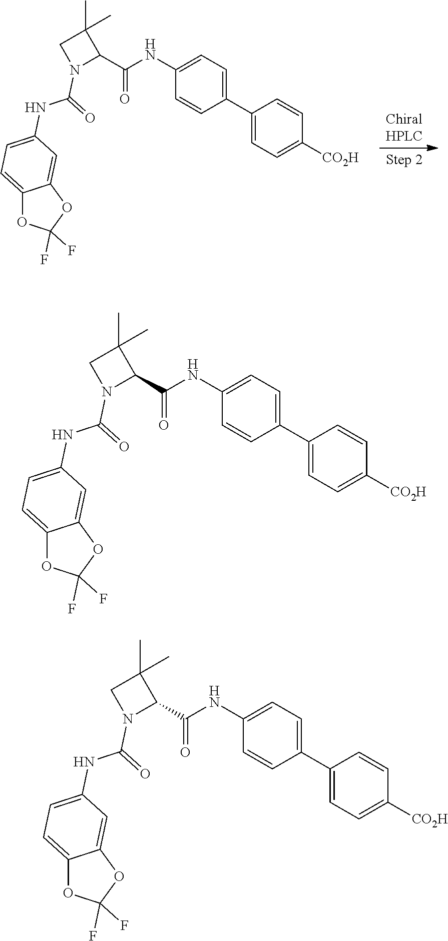 [SMILES: CC1(C)CN(C(=O)Nc2ccc3c(c2)OC(F)(F)O3)C1C(=O)Nc1ccc(-c2ccc(C(=O)O)cc2)cc1 ; CC1(C)CN(C(=O)Nc2ccc3c(c2)OC(F)(F)O3)[C@@H]1C(=O)Nc1ccc(-c2ccc(C(=O)O)cc2)cc1 ; CC1(C)CN(C(=O)Nc2ccc3c(c2)OC(F)(F)O3)[C@H]1C(=O)Nc1ccc(-c2ccc(C(=O)O)cc2)cc1]

4′-(1-((2,2-difluorobenzo[d][1,3]dioxol-5-yl)carbamoyl)-3,3-dimethylazetidine-2-carboxamido)-[1,1′-biphenyl]-4-carboxylic acid (100 mg) was resolved by preparative chiral HPLC (conditions: column: CHIRAL ART Cellulose-SC, 2*25 cm, 5 μm; mobile phase A: MTBE (0.1% formic acid), mobile phase B: methanol; flow rate: 40 mL/min; gradient (B %) isocratic 50% B; detector: 220/254 nm; RT1 (min): 5.7; RT2 (min): 7.9) to afford (Compound 299a; Method BR, Peak 1, 0.51 min) (S)-4′-(1-((2,2-difluorobenzo[d][1,3]dioxol-5-yl)carbamoyl)-3,3-dimethylazetidine-2-carboxamido)-[1,1′-biphenyl]-4-carboxylic acid (1^{st} peak, 37.8 mg, 38%) as a white solid and (Compound 299b; Method BR, Peak 2, 1.81 min) (R)-4′-(1-((2,2-difluorobenzo[d][1,3]dioxol-5-yl)carbamoyl)-3,3-dimethylazetidine-2-carboxamido)-[1,1′-biphenyl]-4-carboxylic acid (2^{nd} peak, 31.6 mg, 32%) as a white solid.

| Cmpd # | Characterization Data |
|---|---|
| 299a | LCMS (ESI) [M + H]⁺: 524.2 |
| | ¹H NMR (400 MHz, CD₃OD) δ 8.07 |
| | (d, J = 8.4 Hz, 2H), 7.78-7.65 (m, 6H), 7.51 (d, J = |
| | 2.1 Hz, 1H), 7.18-7.04 (m, 2H), 4.50 (s, 1H), 3.86 (d, |
| | J = 7.3 Hz, 1H), 3.78 (d, J = 7.4 Hz, 1H), 1.48 |
| | (s, 3H), 1.26 (s, 3H). |
| 299b | LCMS (ESI) [M + H]⁺: 524.2 |
| | ¹H NMR (400 MHz, CD₃OD) δ 8.08 (d, J = |
| | 8.2 Hz, 2H), 7.77-7.64 (m, 6H), 7.51 (d, J = 2.0 Hz, |
| | 1H), 7.16-7.05 (m, 2H), 4.50 (s, 1H), 3.86 (d, J = 7.3 Hz, 1H), |
| | 3.79 (d, J = 7.3 Hz, 1H), 1.48 (s, 3H), 1.26 (s, 3H). |

### Example 302: (S)-5-(4-(1-((3,4-dichlorobenzyl)carbamoyl)-3,3-dimethylazetidine-2-carboxamido)phenyl)-6-methylpicolinic acid and (R)-5-(4-(1-((3,4-dichlorobenzyl)carbamoyl)-3,3-dimethylazetidine-2-carboxamido)phenyl)-6-methylpicolinic acid

### Step 1: 5-(4-(1-((3,4-dichlorobenzyl)carbamoyl)-3,3-dimethylazetidine-2-carboxamido)phenyl)-6-methylpicolinic acid

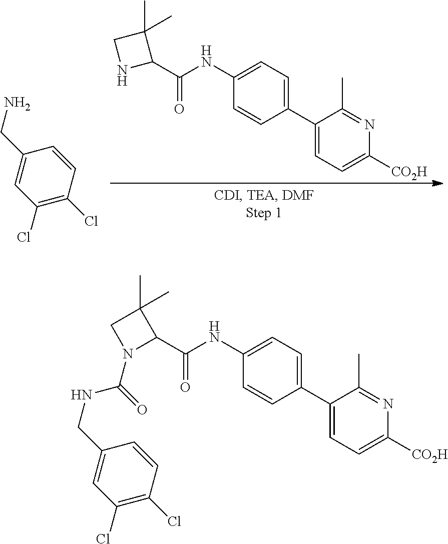 [SMILES: NCc1ccc(Cl)c(Cl)c1 ; Cc1nc(C(=O)O)ccc1-c1ccc(NC(=O)C2NCC2(C)C)cc1 ; Cc1nc(C(=O)O)ccc1-c1ccc(NC(=O)C2N(C(=O)NCc3ccc(Cl)c(Cl)c3)CC2(C)C)cc1]

A solution of (3,4-dichlorophenyl)methanamine (56.9 mg, 0.290 mmol), 5-(4-(3,3-dimethylazetidine-2-carboxamido)phenyl)-6-methylpicolinic acid (50.0 mg, 0.14 mmol), triethylamine (44.7 mg, 0.440 mmol) and CDI (47.7 mg, 0.290 mmol) in DMF (2.0 mL) was stirred at 25° C. for 1 hour. Upon completion, the mixture was concentrated under reduced pressure. The crude residue was directly purified by reverse phase flash chromatography (conditions: column: C18 silica gel; mobile phase A: water (0.1% formic acid); mobile phase B: MeCN; gradient (B %): 0%-100% over 25 min; detector: UV 254/220 nm) to give 5-(4-(1-((3,4-dichlorobenzyl)carbamoyl)-3,3-dimethylazetidine-2-carboxamido)phenyl)-6-methylpicolinic acid (60 mg, 72%) as an off-white solid. LCMS (ESI) [M+H]⁺: 541.2.

### Step 2: (S)-5-(4-(1-((3,4-dichlorobenzyl)carbamoyl)-3,3-dimethylazetidine-2-carboxamido)phenyl)-6-methylpicolinic acid and (R)-5-(4-(1-((3,4-dichlorobenzyl)carbamoyl)-3,3-dimethylazetidine-2-carboxamido)phenyl)-6-methylpicolinic acid

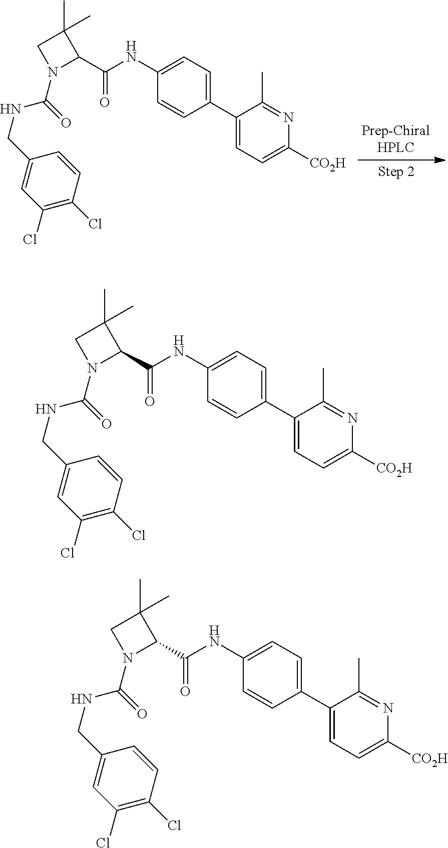

5-(4-(1-((3,4-dichlorobenzyl)carbamoyl)-3,3-dimethylazetidine-2-carboxamido)phenyl)-6-methylpicolinic acid (60 mg) was resolved by preparative chiral HPLC (conditions: column: CHIRAL ART Cellulose-SB, 3*25 cm, 5 μm; mobile phase A: hexane (0.1% formic acid); mobile phase B: ethanol: flow rate: 40 mL/min; gradient (B %): isocratic 50% B; detector: 220/254 nm; RT1 (min): 15.4; RT2 (min): 21.3) to afford (Compound 302a; Method BI, Peak 1, 1.30 min) (S)-5-(4-(1-((3,4-dichlorobenzyl)carbamoyl)-3,3-dimethylazetidine-2-carboxamido)phenyl)-6-methylpicolinic acid (1^{st} peak, 17.1 mg, 28%) as a white solid, and (Compound 302b; Method BI, Peak 2, 1.68 min) (R)-5-(4-(1-((3,4-dichlorobenzyl)carbamoyl)-3,3-dimethylazetidine-2-carboxamido)phenyl)-6-methylpicolinic acid (2^{nd} peak, 16.2 mg, 27%) as a white solid.

| Cmpd # | Characterization Data |
|---|---|
| 302a | LCMS (ESI) [M + H]⁺: 541.2 |
| | ¹H NMR (400 MHz, CD₃OD) δ 8.05 |
| | (d, J = 7.9 Hz, 1H), 7.85 (d, J = 7.9 Hz, 1H), 7.76 (d, J = |
| | 8.6 Hz, 2H), 7.52 (d, J = 2.1 Hz, 1H), 7.45 (d, J = |
| | 8.3 Hz, 1H), 7.39 (d, J = 8.6 Hz, 2H), 7.26 (dd, |
| | J = 8.3, 2.1 Hz, 1H), 4.41 (s, 1H), 4.33 (q, J = |
| | 15.7 Hz, 2H), 3.74 (d, J = 7.3 Hz, 1H), 3.66 (d, J = |
| | 7.2 Hz, 1H), 2.58 (s, 3H), 1.45 (s, 3H), 1.24 (s, 3H). |
| 302b | LCMS (ESI) [M + H]⁺: 541.2 |
| | ¹H NMR (400 MHz, CD₃OD) δ |
| | 8.04 (d, J = 7.9 Hz, 1H), 7.84 (d, J = 7.9 Hz, 1H), 7.75 (d, J = |
| | 8.5 Hz, 2H), 7.52 (d, J = 2.0 Hz, 1H), 7.45 (d, J = |
| | 8.3 Hz, 1H), 7.39 (d, J = 8.6 Hz, 2H), 7.26 (dd, |
| | J = 8.3, 2.0 Hz, 1H), 4.41 (s, 1H), 4.33 (q, J = 15.7 Hz, |
| | 2H), 3.74 (d, J = 7.2 Hz, 1H), 3.66 (d, J = |
| | 7.3 Hz, 1H), 2.57 (s, 3H), 1.45 (s, 3H), 1.24 (s, 3H). |

### Example 303: (S)-5-(4-(1-((4-(tert-butyl)benzyl)carbamoyl)-3,3-dimethylazetidine-2-carboxamido)phenyl)-6-methylpicolinic acid and (R)-5-(4-(1-((4-(tert-butyl)benzyl)carbamoyl)-3,3-dimethylazetidine-2-carboxamido)phenyl)-6-methylpicolinic acid

### Step 1: 5-(4-(1-((4-(tert-butyl)benzyl)carbamoyl)-3,3-dimethylazetidine-2-carboxamido)phenyl)-6-methylpicolinic acid

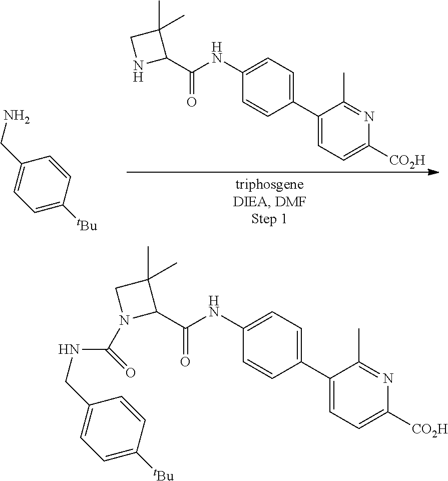 [SMILES: CC(C)(C)c1ccc(CN)cc1 ; Cc1nc(C(=O)O)ccc1-c1ccc(NC(=O)C2NCC2(C)C)cc1 ; Cc1nc(C(=O)O)ccc1-c1ccc(NC(=O)C2N(C(=O)NCc3ccc(C(C)(C)C)cc3)CC2(C)C)cc1]

A solution of (4-(tert-butyl)phenyl)methanamine (43.2 mg, 0.265 mmol), 5-(4-(3,3-dimethylazetidine-2-carboxamido)phenyl)-6-methylpicolinic acid (60.0 mg, 0.177 mmol), DIEA (68.5 mg, 0.531 mmol) and triphosgene (20.9 mg, 0.071 mmol) in DMF (2.0 mL) was stirred at 25° C. for 1 hour. Upon completion, the mixture was concentrated under reduced pressure. The crude residue was directly purified by reverse phase flash chromatography (conditions: column: C18 silica gel; mobile phase A: water (0.1% formic acid); mobile phase B: MeCN; gradient (B %): 0%-100% over 25 min; detector: UV 254/220 nm) to give 5-(4-(1-((4-(tert-butyl)benzyl)carbamoyl)-3,3-dimethylazetidine-2-carboxamido)phenyl)-6-methylpicolinic acid (60 mg, 64%) as an off-white solid. LCMS (ESI) [M+H]⁺: 529.2.

### Step 2: (S)-5-(4-(1-((4-(tert-butyl)benzyl)carbamoyl)-3,3-dimethylazetidine-2-carboxamido)phenyl)-6-methylpicolinic acid and (R)-5-(4-(1-((4-(tert-butyl)benzyl)carbamoyl)-3,3-dimethylazetidine-2-carboxamido)phenyl)-6-methylpicolinic acid

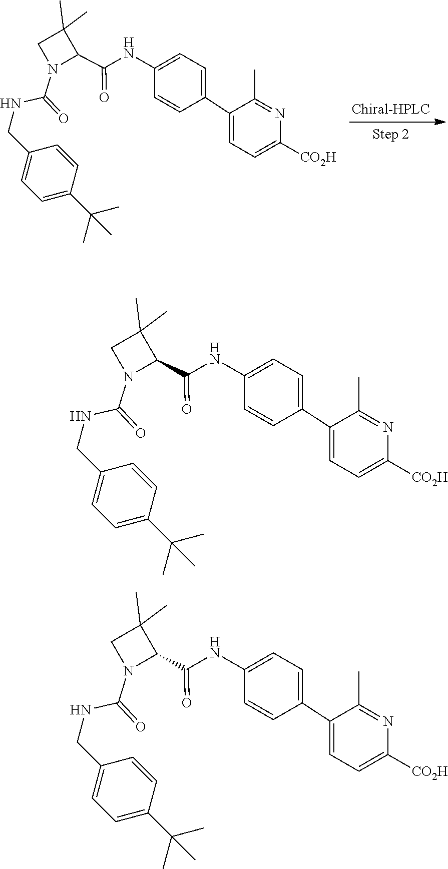

5-(4-(1-((4-(tert-butyl)benzyl)carbamoyl)-3,3-dimethylazetidine-2-carboxamido)phenyl)-6-methylpicolinic acid (60 mg) was resolved by preparative chiral HPLC (conditions: CHIRAL ART Cellulose-SB, 3*25 cm, 5 μm; mobile phase A: hexane (0.1% formic acid); mobile phase B: ethanol: flow rate: 40 mL/min; gradient (B %): isocratic, 50% B; detector: 220/254 nm; RT1 (min): 12.4; RT2 (min): 18.6) to afford (Compound 303a; Method BI, Peak 1, 1.16 min) (S)-5-(4-(1-((4-(tert-butyl)benzyl)carbamoyl)-3,3-dimethylazetidine-2-carboxamido)phenyl)-6-methylpicolinic acid (1^{st} peak, 14.2 mg, 24%) as a white solid, and (Compound 303b; Method BI, Peak 2, 1.68 min) (R)-5-(4-(1-((4-(tert-butyl)benzyl)carbamoyl)-3,3-dimethylazetidine-2-carboxamido)phenyl)-6-methylpicolinic acid (2^{nd} peak, 15.6 mg, 26%) as a white solid.

| Cmpd # | Characterization Data |
|---|---|
| 303a | LCMS (ESI) [M + H]⁺: 529.3 |
| | ¹H NMR (400 MHz, CD₃OD) δ 8.06 (d, J = |
| | 7.9 Hz, 1H), 7.86 (d, J = 7.8 Hz, 1H), 7.76 (d, J = |
| | 8.5 Hz, 2H), 7.39 (d, J = 8.4 Hz, 2H), 7.35 (d, J = |
| | 8.3 Hz, 2H), 7.25 (d, J = 8.3 Hz, 2H), 4.43- |
| | 4.23 (m, 3H), 3.71 (d, J = 7.2 Hz, 1H), 3.65 (d, J = |
| | 7.3 Hz, 1H), 2.58 (s, 3H), 1.43 (s, 3H), 1.29 |
| | (s, 9H), 1.24 (s, 3H). |
| 303b | LCMS (ESI) [M + H]⁺: 529.3 |
| | ¹H NMR (400 MHz, CD₃OD) δ |
| | 8.06 (d, J = 7.9 Hz, 1H), 7.86 (d, J = 7.6 Hz, 1H), 7.76 (d, J = |
| | 8.5 Hz, 2H), 7.38 (d, J = 8.2 Hz, 2H), 7.34 (d, J = |
| | 8.3 Hz, 2H), 7.25 (d, J = 8.4 Hz, 2H), 4.43-4.24 (m, 3H), |
| | 3.71 (d, J = 7.2 Hz, 1H), 3.65 (d, J = 7.3 Hz, 1H), |
| | 2.58 (s, 3H), 1.43 (s, 3H), 1.29 (s, 9H), 1.24 (s, 3H). |

### Example 304: (S)-5-(4-(1-((3-fluoro-4-(trifluoromethyl)benzyl)carbamoyl)-3,3-dimethylazetidine-2-carboxamido)phenyl)-6-methylpicolinic acid and (R)-5-(4-(1-((3-fluoro-4-(trifluoromethyl)benzyl)carbamoyl)-3,3-dimethylazetidine-2-carboxamido)phenyl)-6-methylpicolinic acid

### Step 1: 5-(4-(1-((3-fluoro-4-(trifluoromethyl)benzyl)carbamoyl)-3,3-dimethylazetidine-2-carboxamido)phenyl)-6-methylpicolinic acid

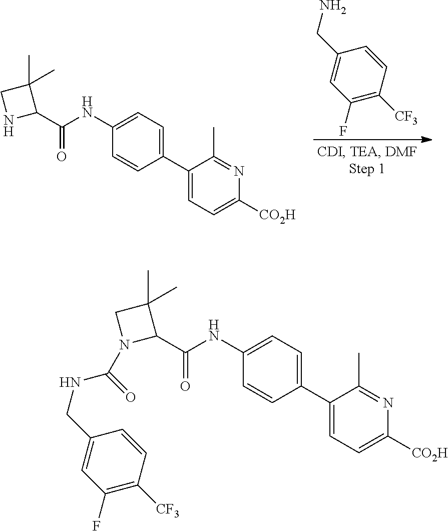 [SMILES: Cc1nc(C(=O)O)ccc1-c1ccc(NC(=O)C2NCC2(C)C)cc1 ; NCc1ccc(C(F)(F)F)c(F)c1 ; Cc1nc(C(=O)O)ccc1-c1ccc(NC(=O)C2N(C(=O)NCc3ccc(C(F)(F)F)c(F)c3)CC2(C)C)cc1]

A solution of (3-fluoro-4-(trifluoromethyl)phenyl)methanamine (91.0 mg, 0.472 mmol), 5-(4-(3,3-dimethylazetidine-2-carboxamido)phenyl)-6-methylpicolinic acid (80.0 mg, 0.236 mmol), triethylamine (71.5 mg, 0.708 mmol) and CDI (76.4 mg, 0.472 mmol) in DMF (3.0 mL) was stirred at 25° C. for 1 hour. Upon completion, the mixture was concentrated under reduced pressure. The crude residue was directly purified by reverse phase flash chromatography (conditions: column: C18 silica gel; mobile phase A: water (0.1% formic acid); mobile phase B: MeCN; gradient (B %): 0%-100% over 25 min; detector: UV 254/220 nm) to give 5-(4-(1-((3-fluoro-4-(trifluoromethyl)benzyl)carbamoyl)-3,3-dimethylazetidine-2-carboxamido)phenyl)-6-methylpicolinic acid (80 mg, 60%) as an off-white solid. LCMS (ESI) [M+H]⁺: 559.1.

### Step 2: (S)-5-(4-(1-((3-fluoro-4-(trifluoromethyl)benzyl)carbamoyl)-3,3-dimethylazetidine-2-carboxamido)phenyl)-6-methylpicolinic acid and (R)-5-(4-(1-((3-fluoro-4-(trifluoromethyl)benzyl)carbamoyl)-3,3-dimethylazetidine-2-carboxamido)phenyl)-6-methylpicolinic acid

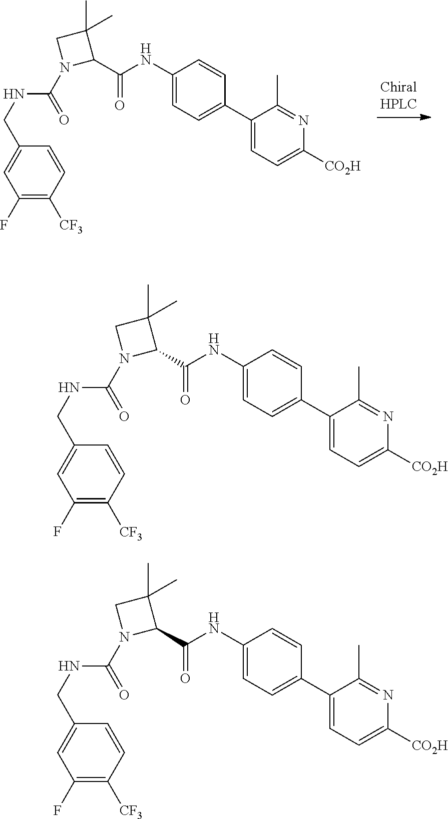

5-(4-(1-((3-fluoro-4-(trifluoromethyl)benzyl)carbamoyl)-3,3-dimethylazetidine-2-carboxamido)phenyl)-6-methylpicolinic acid (80 mg) was resolved by preparative chiral HPLC (conditions: column: CHIRAL ART Cellulose-SZ, 3*25 cm, 5 μm; mobile phase A: MTBE (0.1% formic acid), mobile phase B: methanol; flow rate: 40 mL/min; gradient (B %): isocratic, 50% B; detector: 220/254 nm; RT1 (min): 10.6; RT2 (min): 17.3) to afford (Compound 304a; Method BL, Peak 1, 1.02 min) (S)-5-(4-(1-((3-fluoro-4-(trifluoromethyl)benzyl)carbamoyl)-3,3-dimethylazetidine-2-carboxamido)phenyl)-6-methylpicolinic acid (1^{st} peak, 18.0 mg, 23%) as a white solid, and (Compound 304b; Method BL, Peak 2, 1.92 min) (R)-5-(4-(1-((3-fluoro-4-(trifluoromethyl)benzyl)carbamoyl)-3,3-dimethylazetidine-2-carboxamido)phenyl)-6-methylpicolinic acid (2^{nd} peak, 20.0 mg, 25%) as a white solid.

| Cmpd # | Characterization Data |
|---|---|
| 304a | LCMS (ESI) [M + H]⁺: 559.2 |
| | ¹H NMR (400 MHz, CD₃OD) δ 8.10 |
| | (d, J = 21.1 Hz, 1H), 7.86 (d, J = 7.6 Hz, 1H), 7.76 (d, |
| | J = 8.0 Hz, 2H), 7.62 (t, J = 7.8 Hz, 1H), 7.43-7.27 |
| | (m, 4H), 4.50-4.31 (m, 3H), 3.76 (d, J = 7.2 Hz, 1H), 3.68 |
| | (d, J = 7.2 Hz, 1H), 2.57 (s, 3H), 1.46 (s, 3H), 1.25 (s, 3H). |
| 304b | LCMS (ESI) [M + H]⁺: 559.2 |
| | ¹H NMR (400 MHz, CD₃OD) δ |
| | 8.07 (d, J = 7.8 Hz, 1H), 7.86 (d, J = 7.7 Hz, 1H), 7.76 |
| | (d, J = 8.2 Hz, 2H), 7.62 (t, J = 7.8 Hz, 1H), 7.39 |
| | (d, J = 8.1 Hz, 2H), 7.36-7.27 (m, 2H), 4.50-4.35 |
| | (m, 3H), 3.76 (d, J = 7.1 Hz, 1H), 3.68 |
| | (d, J = 7.2 Hz, 1H), 2.58 (s, 3H), 1.46 |
| | (s, 3H), 1.25 (s, 3H). |

### Example 305: (S)-5-(4-(1-((3-fluoro-4-methylbenzyl)carbamoyl)-3,3-dimethylazetidine-2-carboxamido)phenyl)-6-methylpicolinic acid and (R)-5-(4-(1-((3-fluoro-4-methylbenzyl)carbamoyl)-3,3-dimethylazetidine-2-carboxamido)phenyl)-6-methylpicolinic acid

### Step 1: 5-(4-(1-((3-fluoro-4-methylbenzyl)carbamoyl)-3,3-dimethylazetidine-2-carboxamido)phenyl)-6-methylpicolinic acid

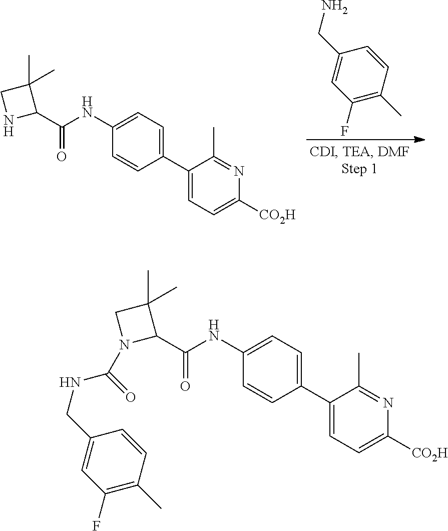

A solution of (3-fluoro-4-methylphenyl)methanamine (57.4 mg, 0.412 mmol), 5-(4-(3,3-dimethylazetidine-2-carboxamido)phenyl)-6-methylpicolinic acid (70.0 mg, 0.206 mmol), triethylamine (62.6 mg, 0.618 mmol) and CDI (66.8 mg, 0.412 mmol) in DMF (3.0 mL) was stirred at 25° C. for 1 hour. Upon completion, the mixture was concentrated under reduced pressure. The crude residue was directly purified by reverse phase flash chromatography (conditions: column: C18 silica gel; mobile phase A: water (0.1% formic acid); mobile phase B: MeCN; gradient (B %): 0%-100% over 25 min; detector: UV 254/220 nm) to give 5-(4-(1-((3-fluoro-4-methylbenzyl)carbamoyl)-3,3-dimethylazetidine-2-carboxamido)phenyl)-6-methylpicolinic acid (80 mg, 76%) as an off-white solid. LCMS (ESI) [M+H]⁺: 505.2.

### Step 2: (S)-5-(4-(1-((3-fluoro-4-methylbenzyl)carbamoyl)-3,3-dimethylazetidine-2-carboxamido)phenyl)-6-methylpicolinic acid and (R)-5-(4-(1-((3-fluoro-4-methylbenzyl)carbamoyl)-3,3-dimethylazetidine-2-carboxamido)phenyl)-6-methylpicolinic acid

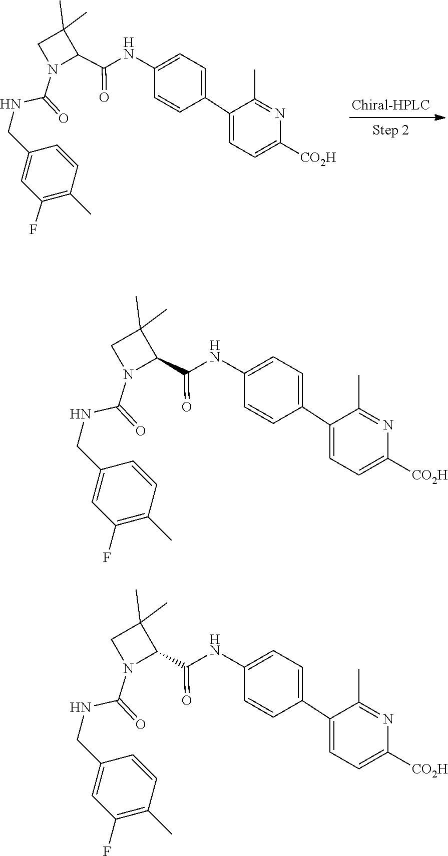

5-(4-(1-((3-fluoro-4-methylbenzyl)carbamoyl)-3,3-dimethylazetidine-2-carboxamido)phenyl)-6-methylpicolinic acid (80 mg) was resolved by preparative chiral HPLC (conditions: column: CHIRAL ART Cellulose-SZ, 3*25 cm, 5 μm; mobile phase A: MTBE (0.1% formic acid); mobile phase B: methanol; flow rate: 40 mL/min; gradient (B %): isocratic, 50% B; detector: 220/254 nm; RT1 (min): 13.7; RT2 (min): 17.5) to afford (Compound 305a; Method BU, Peak 1, 1.42 min) (S)-5-(4-(1-((3-fluoro-4-methylbenzyl)carbamoyl)-3,3-dimethylazetidine-2-carboxamido)phenyl)-6-methylpicolinic acid (1^{st} peak, 30.4 mg, 39%) as a white solid, and (Compound 305b; Method BU, Peak 2, 1.66 min) (R)-5-(4-(1-((3-fluoro-4-methylbenzyl)carbamoyl)-3,3-dimethylazetidine-2-carboxamido)phenyl)-6-methylpicolinic acid (2^{nd} peak, 26.5 mg, 33%) as a white solid.

| Cmpd # | Characterization Data |
|---|---|
| 305a | LCMS (ESI) [M + H]⁺: 505.3 |
| | ¹H NMR (400 MHz, CD₃OD) δ 8.06 |
| | (s, 1H), 7.86 (s, 1H), 7.76 (d, J = 7.5 Hz, 2H), 7.38 (d, J = |
| | 8.0 Hz, 2H), 7.15 (t, J = 7.8 Hz, 1H), 7.02 (d, J = |
| | 11.0 Hz, 2H), 4.41 (s, 1H), 4.40-4.23 (m, 2H), |
| | 3.73 (d, J = 7.2 Hz, 1H), 3.65 (d, J = 7.2 Hz, 1H), |
| | 2.58 (s, 3H), 2.22 (d, J = 1.9 Hz, 3H), 1.44 (s, |
| | 3H), 1.24 (s, 3H). |
| 305b | LCMS (ESI) [M + H]⁺: 505.2 |
| | ¹H NMR (400 MHz, CD₃OD) δ 8.06 |
| | (d, J = 7.8 Hz, 1H), 7.86 (d, J = 7.4 Hz, 1H), 7.76 (d, J = |
| | 8.1 Hz, 2H), 7.39 (d, J = 8.0 Hz, 2H), 7.15 (t, J = |
| | 7.8 Hz, 1H), 7.04-6.98 (m, 2H), 4.41 (s, 1H), 4.40-4.22 |
| | (m, 2H), 3.73 (d, J = 7.3 Hz, 1H), 3.65 (d, J = 7.2 Hz, |
| | 1H), 2.58 (s, 3H), 2.22 (s, 3H), 1.44 (s, 3H), 1.24 (s, 3H). |

### Example 306: (S)-5-(4-(1-((2-fluoro-4-(trifluoromethyl)benzyl)carbamoyl)-3,3-dimethylazetidine-2-carboxamido)phenyl)-6-methylpicolinic acid and (R)-5-(4-(1-((2-fluoro-4-(trifluoromethyl)benzyl)carbamoyl)-3,3-dimethylazetidine-2-carboxamido)phenyl)-6-methylpicolinic acid

### Step 1: 5-(4-(1-((2-fluoro-4-(trifluoromethyl)benzyl)carbamoyl)-3,3-dimethylazetidine-2-carboxamido)phenyl)-6-methylpicolinic acid

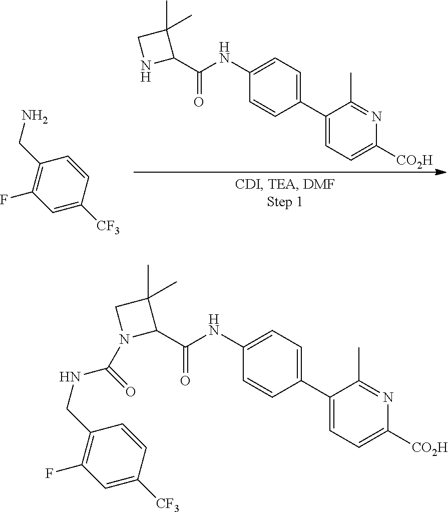 [SMILES: NCc1ccc(C(F)(F)F)cc1F ; Cc1nc(C(=O)O)ccc1-c1ccc(NC(=O)C2NCC2(C)C)cc1 ; Cc1nc(C(=O)O)ccc1-c1ccc(NC(=O)C2N(C(=O)NCc3ccc(C(F)(F)F)cc3F)CC2(C)C)cc1]

A solution of (2-fluoro-4-(trifluoromethyl)phenyl)methanamine (56.9 mg, 0.290 mmol), 5-(4-(3,3-dimethylazetidine-2-carboxamido)phenyl)-6-methylpicolinic acid (50.0 mg, 0.140 mmol), triethylamine (44.7 mg, 0.44 mmol) and CDI (47.7 mg, 0.290 mmol) in DMF (3.0 mL) was stirred at 25° C. for 1 hour. Upon completion, the mixture was concentrated under reduced pressure. The crude residue was directly purified by reverse phase flash chromatography (conditions: column: C18 silica gel; mobile phase A: water (0.1% formic acid); mobile phase B: MeCN; gradient (B %): 0%-100% over 25 min; detector: UV 254/220 nm) to give 5-(4-(1-((2-fluoro-4-(trifluoromethyl)benzyl)carbamoyl)-3,3-dimethylazetidine-2-carboxamido)phenyl)-6-methylpicolinic acid (60.0 mg, 72%) as an off-white solid. LCMS (ESI) [M+H]⁺: 559.2.

### Step 2: (S)-5-(4-(1-((2-fluoro-4-(trifluoromethyl)benzyl)carbamoyl)-3,3-dimethylazetidine-2-carboxamido)phenyl)-6-methylpicolinic acid and (R)-5-(4-(1-((2-fluoro-4-(trifluoromethyl)benzyl)carbamoyl)-3,3-dimethylazetidine-2-carboxamido)phenyl)-6-methylpicolinic acid

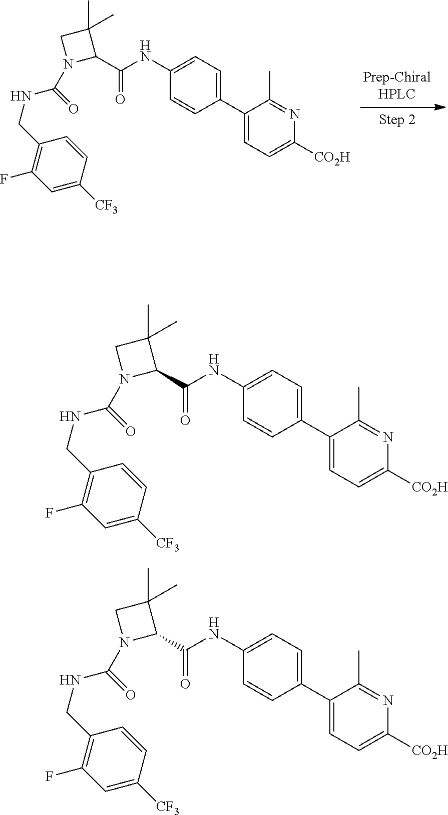

5-(4-(1-((2-fluoro-4-(trifluoromethyl)benzyl)carbamoyl)-3,3-dimethylazetidine-2-carboxamido)phenyl)-6-methylpicolinic acid (60 mg) was resolved by preparative chiral HPLC (conditions: column: CHIRAL ART Cellulose-SZ, 3*25 cm, 5 μm; mobile phase A: MtBE (0.1% formic acid), mobile phase B: methanol; flow rate: 40 mL/min; gradient (B %): isocratic, 50% B; detector: 220/254 nm; RT1 (min): 7.3; RT2 (min): 22.5) to afford (Compound 306a; Method BU, Peak 1, 0.93 min) (S)-5-(4-(1-((2-fluoro-4-(trifluoromethyl)benzyl)carbamoyl)-3,3-dimethylazetidine-2-carboxamido)phenyl)-6-methylpicolinic acid (1^{st} peak, 27.6 mg, 46%) as a white solid, and (Compound 306b; Method BU, Peak 2, 2.19 min) (R)-5-(4-(1-((2-fluoro-4-(trifluoromethyl)benzyl)carbamoyl)-3,3-dimethylazetidine-2-carboxamido)phenyl)-6-methylpicolinic acid (2^{nd} peak, 22.6 mg, 37%) as a white solid.

| Cmpd # | Characterization Data |
|---|---|
| 306a | LCMS (ESI) [M + H]⁺: 559.3 |
| | ¹H NMR (400 MHz, CD₃OD) δ 8.07 (d, J = |
| | 7.9 Hz, 1H), 7.86 (d, J = 7.9 Hz, 1H), 7.76 (d, J = 8.5 Hz, 2H), |
| | 7.66 (t, J = 7.7 Hz, 1H), 7.48 (d, J = 8.0 Hz, 1H), 7.40 (dd, |
| | J = 9.0, 2.4 Hz, 3H), 4.57-4.39 (m, 3H), 3.76 (d, J = 7.2 Hz, |
| | 1H), 3.68 (d, J = 7.3 Hz, 1H), 2.58 (s, 3H), |
| | 1.46 (s, 3H), 1.25 (s, 3H). |
| 306b | LCMS (ESI) [M + H]⁺: 559.3 |
| | ¹H NMR (400 MHz, CD₃OD) δ 8.06 |
| | (d, J = 7.9 Hz, 1H), 7.86 (d, J = 7.8 Hz, 1H), 7.76 (d, J = |
| | 8.4 Hz, 2H), 7.66 (t, J = 7.7 Hz, 1H), 7.48 (d, J = 8.1 Hz, |
| | 1H), 7.40 (dd, J = 9.1, 2.7 Hz, 3H), 4.57-4.38 |
| | (m, 3H), 3.76 (d, J = 7.2 Hz, 1H), 3.68 (d, J = |
| | 7.2 Hz, 1H), 2.58 (s, 3H), 1.46 (s, 3H), 1.25 (s, 3H). |

### Example 307: (S)-5-(4-(1-((4-chloro-2-fluorobenzyl)carbamoyl)-3,3-dimethylazetidine-2-carboxamido)phenyl)-6-methylpicolinic acid and (R)-5-(4-(1-((4-chloro-2-fluorobenzyl)carbamoyl)-3,3-dimethylazetidine-2-carboxamido)phenyl)-6-methylpicolinic acid

### Step 1: 5-(4-(1-((4-chloro-2-fluorobenzyl)carbamoyl)-3,3-dimethylazetidine-2-carboxamido)phenyl)-6-methylpicolinic acid

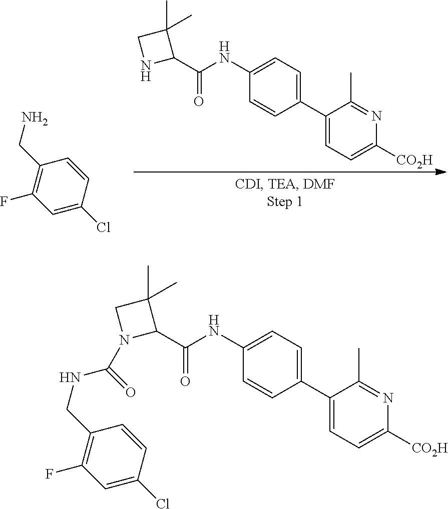 [SMILES: NCc1ccc(Cl)cc1F ; Cc1nc(C(=O)O)ccc1-c1ccc(NC(=O)C2NCC2(C)C)cc1 ; Cc1nc(C(=O)O)ccc1-c1ccc(NC(=O)C2N(C(=O)NCc3ccc(Cl)cc3F)CC2(C)C)cc1]

A solution of (4-chloro-2-fluorophenyl)methanamine (75.2 mg, 0.472 mmol), 5-(4-(3,3-dimethylazetidine-2-carboxamido)phenyl)-6-methylpicolinic acid (80.0 mg, 0.236 mmol), triethylamine (71.5 mg, 0.708 mmol) and CDI (76.4 mg, 0.472 mmol) in DMF (3.0 mL) was stirred at 25° C. for 1 hour. Upon completion, the mixture was concentrated under reduced pressure. The crude residue was purified by reversed phase flash chromatography (conditions: column: C18 silica gel; mobile phase A: water (0.1% formic acid); mobile phase B: MeCN; gradient (B %): 0%-100% over 25 min; detector: UV 254/220 nm) to give 5-(4-(1-((4-chloro-2-fluorobenzyl)carbamoyl)-3,3-dimethylazetidine-2-carboxamido)phenyl)-6-methylpicolinic acid (60 mg, 48%) as an off-white solid. LCMS (ESI) [M+H]⁺: 505.2.

### Step 2: (S)-5-(4-(1-((4-chloro-2-fluorobenzyl)carbamoyl)-3,3-dimethylazetidine-2-carboxamido)phenyl)-6-methylpicolinic acid and (R)-5-(4-(1-((4-chloro-2-fluorobenzyl)carbamoyl)-3,3-dimethylazetidine-2-carboxamido)phenyl)-6-methylpicolinic acid

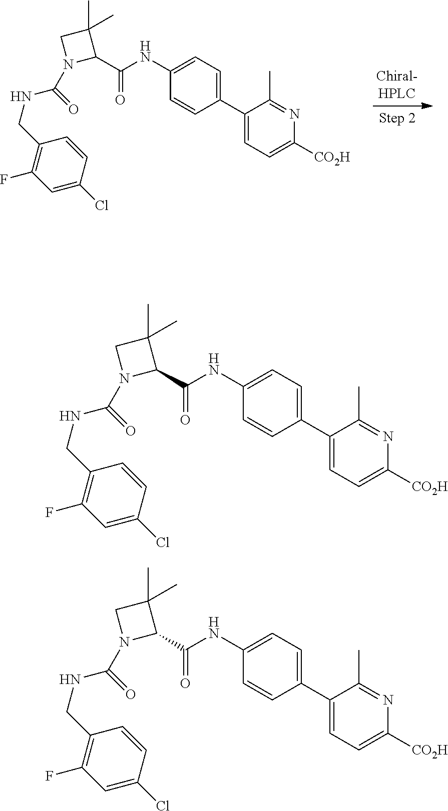

5-(4-(1-((4-chloro-2-fluorobenzyl)carbamoyl)-3,3-dimethylazetidine-2-carboxamido)phenyl)-6-methylpicolinic acid (60 mg) was resolved by preparative chiral HPLC (conditions: column: CHIRAL ART Cellulose-SB, 3*25 cm, 5 μm; mobile phase A: hexane (0.1% formic acid); mobile phase B: ethanol: flow rate: 40 mL/min; gradient (B %): isocratic, 50% B; detector: 220/254 nm; RT1 (min): 10.2; RT2 (min): 17.9) to afford (Compound 307a; Method BI, Peak 1, 0.98 min) (S)-5-(4-(1-((4-chloro-2-fluorobenzyl)carbamoyl)-3,3-dimethylazetidine-2-carboxamido)phenyl)-6-methylpicolinic acid (1^{st} peak, 21.2 mg, 42%) as a white solid, and (Compound 307b; Method BI, Peak 2, 1.71 min) (R)-5-(4-(1-((4-chloro-2-fluorobenzyl)carbamoyl)-3,3-dimethylazetidine-2-carboxamido)phenyl)-6-methylpicolinic acid 2^{nd} peak, 18.6 mg, 37%) as a white solid.

| Cmpd # | Characterization Data |
|---|---|
| 307a | LCMS (ESI) [M + H]⁺: 505.3 |
| | ¹H NMR (400 MHz, CD₃OD) δ 8.07 |
| | (d, J = 7.9 Hz, 1H), 7.86 (d, J = 7.8 Hz, 1H), 7.75 (d, J = |
| | 8.4 Hz, 2H), 7.47-7.35 (m, 3H), 7.21-7.10 (m, 2H), |
| | 4.49-4.30 (m, 3H), 3.74 (d, J = 7.3 Hz, 1H), 3.66 |
| | (d, J = 7.2 Hz, 1H), 2.58 (s, 3H), 1.44 (s, 3H), 1.24 (s, 3H). |
| 307b | LCMS (ESI) [M + H]⁺: 505.2 |
| | ¹H NMR (400 MHz, CD₃OD) δ |
| | 8.07 (d, J = 7.9 Hz, 1H), 7.86 (d, J = 7.8 Hz, 1H), 7.79-7.70 (m, |
| | 2H), 7.47-7.34 (m, 3H), 7.16 (ddd, J = 10.3, 8.8, 1.9 Hz, |
| | 2H), 4.49-4.30 (m, 3H), 3.74 (d, J = 7.2 Hz, 1H), 3.66 |
| | (d, J = 7.3 Hz, 1H), 2.58 (s, 3H), 1.44 (s, 3H), 1.24 (s, 3H). |

### Example 308: (S)-5-(4-(3,3-dimethyl-1-((4-(trifluoromethyl)benzyl)carbamoyl)azetidine-2-carboxamido)phenyl)-6-methylpicolinic acid and (R)-5-(4-(3,3-dimethyl-1-((4-(trifluoromethyl)benzyl)carbamoyl)azetidine-2-carboxamido)phenyl)-6-methylpicolinic acid

### Step 1: 5-(4-(3,3-dimethyl-1-((4-(trifluoromethyl)benzyl)carbamoyl)azetidine-2-carboxamido)phenyl)-6-methylpicolinic acid

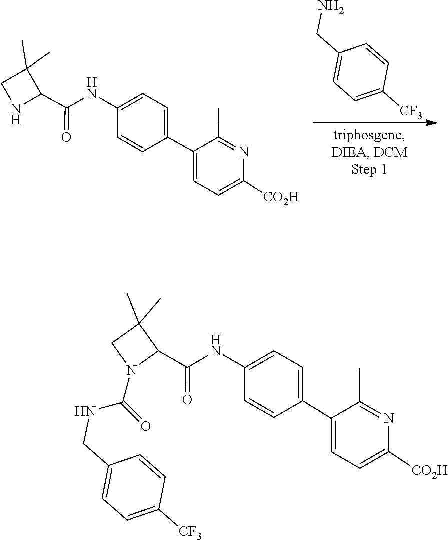 [SMILES: Cc1nc(C(=O)O)ccc1-c1ccc(NC(=O)C2NCC2(C)C)cc1 ; NCc1ccc(C(F)(F)F)cc1 ; Cc1nc(C(=O)O)ccc1-c1ccc(NC(=O)C2N(C(=O)NCc3ccc(C(F)(F)F)cc3)CC2(C)C)cc1]

A solution of (4-(trifluoromethyl)phenyl)methanamine (175 mg, 0.999 mmol), 5-(4-(3,3-dimethylazetidine-2-carboxamido)phenyl)-6-methylpicolinic acid (113 mg, 0.333 mmol), DIEA (430 mg, 3.33 mmol) and triphosgene (98.8 mg, 0.333 mmol) in DMF (3.0 mL) was stirred at 25° C. for 1 hour. Upon completion, the mixture was concentrated under reduced pressure. The crude residue was directly purified by reverse phase flash chromatography (conditions: column: C18 silica gel; mobile phase A: water (0.1% formic acid); mobile phase B: MeCN; gradient (B %): 0%-100% over 25 min; detector: UV 254/220 nm) to give 5-(4-(3,3-dimethyl-1-((4-(trifluoromethyl)benzyl)carbamoyl)azetidine-2-carboxamido)phenyl)-6-methylpicolinic acid (60 mg, 32%) as an off-white solid. LCMS (ESI) [M+H]⁺: 541.2.

### Step 2: (S)-5-(4-(3,3-dimethyl-1-((4-(trifluoromethyl)benzyl)carbamoyl)azetidine-2-carboxamido)phenyl)-6-methylpicolinic acid and (R)-5-(4-(3,3-dimethyl-1-((4-(trifluoromethyl)benzyl)carbamoyl)azetidine-2-carboxamido)phenyl)-6-methylpicolinic acid

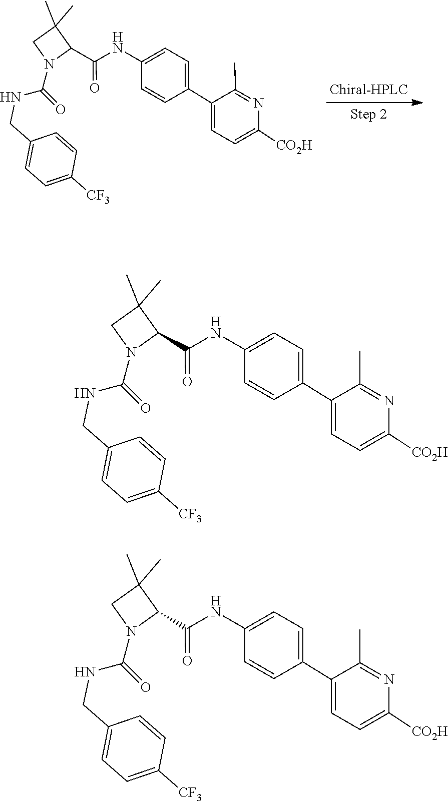 [SMILES: Cc1nc(C(=O)O)ccc1-c1ccc(NC(=O)C2N(C(=O)NCc3ccc(C(F)(F)F)cc3)CC2(C)C)cc1 ; Cc1nc(C(=O)O)ccc1-c1ccc(NC(=O)[C@H]2N(C(=O)NCc3ccc(C(F)(F)F)cc3)CC2(C)C)cc1 ; Cc1nc(C(=O)O)ccc1-c1ccc(NC(=O)[C@@H]2N(C(=O)NCc3ccc(C(F)(F)F)cc3)CC2(C)C)cc1]

5-(4-(3,3-dimethyl-1-((4-(trifluoromethyl)benzyl)carbamoyl)azetidine-2-carboxamido)phenyl)-6-methylpicolinic acid (60 mg) was resolved by preparative chiral HPLC (conditions: column: CHIRAL ART Cellulose-SZ, 3*25 cm, 5 μm; mobile phase A: MTBE (0.1% formic acid); mobile phase B: methanol; flow rate: 40 mL/min; gradient (B %): isocratic, 50% B; detector: 220/254 nm; RT1 (min): 11.4; RT2 (min): 17.3) to afford (Compound 308a; Method BL, Peak 1, 1.26 min) (S)-5-(4-(3,3-dimethyl-1-((4-(trifluoromethyl)benzyl)carbamoyl)azetidine-2-carboxamido)phenyl)-6-methylpicolinic acid (1^{st} peak, 18.0 mg, 30%) as a white solid, and (Compound 308b; Method BL, Peak 2, 1.99 min) (R)-5-(4-(1-((4-chloro-2-fluorobenzyl)carbamoyl)-3,3-dimethylazetidine-2-carboxamido)phenyl)-6-methylpicolinic acid (2^{nd} peak, 17.9 mg, 29%) as a white solid.

| Cmpd # | Characterization Data |
|---|---|
| 308a | LCMS (ESI) [M + H]⁺: 541.2 |
| | ¹H NMR (400 MHz, CD₃OD) δ 8.06 |
| | (d, J = 7.8 Hz, 1H), 7.86 (d, J = 7.6 Hz, 1H), 7.75 (d, J = |
| | 8.1 Hz, 2H), 7.61 (d, J = 8.1 Hz, 2H), 7.53 (d, J = 8.1 Hz, |
| | 2H), 7.39 (d, J = 8.1 Hz, 2H), 4.44 (d, J = 14.0 Hz, 3H), |
| | 3.75 (d, J = 7.2 Hz, 1H), 3.67 (d, J = 7.2 Hz, 1H), |
| | 2.58 (s, 3H), 1.45 (s, 3H), 1.25 (s, 3H). |
| 308b | LCMS (ESI) [M + H]⁺: 541.2 |
| | ¹H NMR (400 MHz, CD₃OD) δ 8.06 |
| | (d, J = 7.9 Hz, 1H), 7.86 (d, J = 7.8 Hz, 1H), 7.75 (d, J = |
| | 8.6 Hz, 2H), 7.61 (d, J = 8.2 Hz, 2H), 7.53 (d, J = |
| | 8.1 Hz, 2H), 7.39 (d, J = 8.6 Hz, 2H), 4.44 (d, J = 14.2 |
| | Hz, 3H), 3.75 (d, J = 7.2 Hz, 1H), 3.67 (d, J = |
| | 7.2 Hz, 1H), 2.58 (s, 3H), 1.45 (s, 3H), 1.25 (s, 3H). |

### Example 309: (S)-5-(4-(1-((4-isobutylbenzyl)carbamoyl)-3,3-dimethylazetidine-2-carboxamido)phenyl)-6-methylpicolinic acid and (R)-5-(4-(1-((4-isobutylbenzyl)carbamoyl)-3,3-dimethylazetidine-2-carboxamido)phenyl)-6-methylpicolinic acid

### Step 1: tert-butyl (4-isobutylbenzyl)carbamate

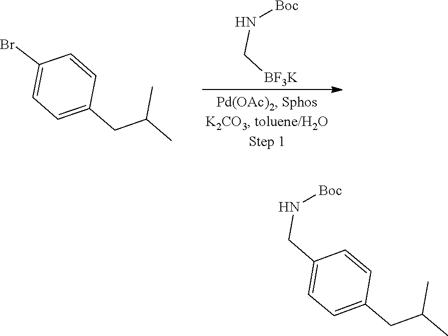

A mixture of 1-bromo-4-isobutylbenzene (3.00 g, 14.1 mmol), tert-butyl ((trifluoro-14-boraneyl)methyl)carbamate, potassium salt (3.30 g, 14.1 mmol), K₂CO₃ (5.80 g, 42.2 mmol), 2-(dicyclohexylphosphino)-2′-methylbiphenyl (578 mg, 1.41 mmol) and palladium (II) acetate (316 mg, 1.41 mmol) in toluene (20 mL) and water (6 mL) was heated to 80° C. for 12 hours. Upon completion, the mixture was concentrated under reduced pressure. The crude residue was directly purified by silica gel column chromatography, eluted with petroleum ether/ethyl acetate (5:1) to afford tert-butyl (4-isobutylbenzyl)carbamate (1.70 g, 48%) as a yellow solid. LCMS (ESI) [M+H]⁺: 264.2.

### Step 2: (4-isobutylphenyl)methanamine

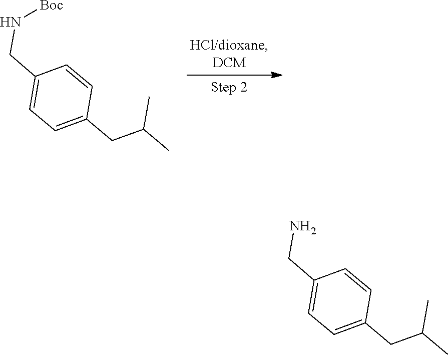 [SMILES: CC(C)Cc1ccc(CNC(=O)OC(C)(C)C)cc1 ; CC(C)Cc1ccc(CN)cc1]

A solution of hydrochloric acid (4.0 mL, 16 mmol, 4.0 M in dioxane) was added to a solution of tert-butyl (4-isobutylbenzyl)carbamate (1.70 g, 6.46 mmol) in dichloromethane (10 mL) at room temperature. After 3 hours, the mixture was concentrated under reduced pressure. The crude residue was purified by reversed-phase flash chromatography (conditions: column: C18 silica gel; mobile phase A: water (0.1% formic acid); mobile phase B: MeCN; gradient (B %): 0%-100% over 25 min; detector: UV 254/220 nm) to afford (4-isobutylphenyl)methanamine (750 mg, 71%) as a white solid. LCMS (ESI) [M+H]⁺: 147.3

### Step 3: 5-(4-(1-((4-isobutylbenzyl)carbamoyl)-3,3-dimethylazetidine-2-carboxamido)phenyl)-6-methylpicolinic acid

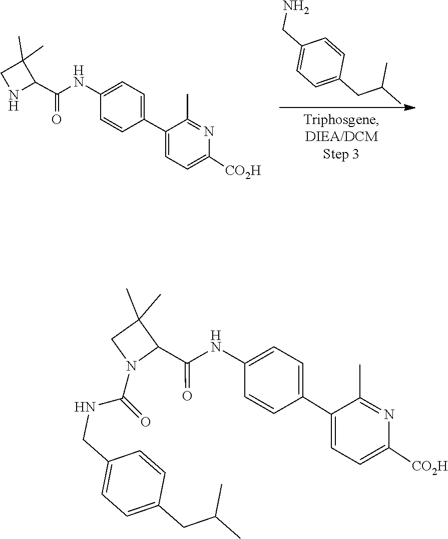 [SMILES: Cc1nc(C(=O)O)ccc1-c1ccc(NC(=O)C2NCC2(C)C)cc1 ; CC(C)Cc1ccc(CN)cc1 ; Cc1nc(C(=O)O)ccc1-c1ccc(NC(=O)C2N(C(=O)NCc3ccc(CC(C)C)cc3)CC2(C)C)cc1]

A solution of 5-(4-(3,3-dimethylazetidine-2-carboxamido)phenyl)-6-methylpicolinic acid (166 mg, 0.490 mmol), (4-isobutylphenyl)methanamine (80.0 mg, 0.490 mmol), DIEA (316 mg, 2.45 mmol) and triphosgene (58.0 mg, 0.196 mmol) in dichloromethane (5.0 mL) was stirred at 25° C. for 1 hour. Upon completion, the mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0%-100% over 25 min; detector: UV 254/220 nm) to afford 5-(4-(1-((4-isobutylbenzyl)carbamoyl)-3,3-dimethylazetidine-2-carboxamido)phenyl)-6-methylpicolinic acid (60.0 mg, 23%) as an off-white solid. LCMS (ESI) [M+H]⁺: 529.3.

### Step 4: (S)-5-(4-(1-((4-isobutylbenzyl)carbamoyl)-3,3-dimethylazetidine-2-carboxamido)phenyl)-6-methylpicolinic acid and (R)-5-(4-(1-((4-isobutylbenzyl)carbamoyl)-3,3-dimethylazetidine-2-carboxamido)phenyl)-6-methylpicolinic acid

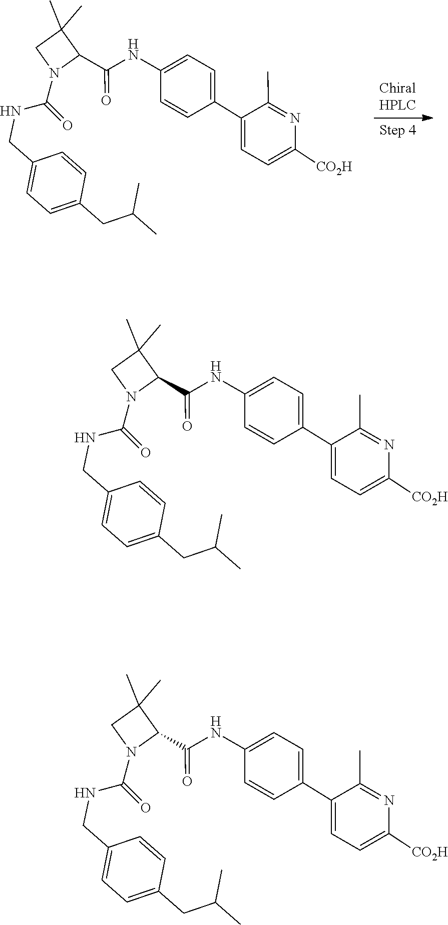 [SMILES: Cc1nc(C(=O)O)ccc1-c1ccc(NC(=O)C2N(C(=O)NCc3ccc(CC(C)C)cc3)CC2(C)C)cc1 ; Cc1nc(C(=O)O)ccc1-c1ccc(NC(=O)[C@H]2N(C(=O)NCc3ccc(CC(C)C)cc3)CC2(C)C)cc1 ; Cc1nc(C(=O)O)ccc1-c1ccc(NC(=O)[C@@H]2N(C(=O)NCc3ccc(CC(C)C)cc3)CC2(C)C)cc1]

5-(4-(1-((4-isobutylbenzyl)carbamoyl)-3,3-dimethylazetidine-2-carboxamido)phenyl)-6-methylpicolinic acid (60 mg) was resolved by preparative chiral HPLC (conditions: column: CHIRAL ART Cellulose-SC, 3*25 cm, 5 μm; mobile phase A: MTBE (0.1% formic acid), mobile phase B: methanol; flow rate: 40 mL/min; gradient (B %): isocratic, 50% B; detector: 220/254 nm; RT1 (min): 15.4; RT2 (min): 30.4) to afford (Compound 309a; Method BV, Peak 1, 1.88 min) (S)-5-(4-(1-((4-isobutylbenzyl)carbamoyl)-3,3-dimethylazetidine-2-carboxamido)phenyl)-6-methylpicolinic acid (1^{st} peak, 11.5 mg, 19%) as a white solid, and (Compound 309b; Method BV, Peak 2, 3.52 min) (R)-5-(4-(1-((4-isobutylbenzyl)carbamoyl)-3,3-dimethylazetidine-2-carboxamido)phenyl)-6-methylpicolinic acid (2^{nd} peak, 17.3 mg, 29%) as a white solid.

| Cmpd # | Characterization Data |
|---|---|
| 309a | LCMS (ESI) [M + H]⁺: 529.3 |
| | ¹H NMR (400 MHz, CD₃OD) δ 8.05 |
| | (d, J = 8.0 Hz, 1H), 7.84 (d, J = 7.8 Hz, 1H), 7.75 (d, J = |
| | 8.4 Hz, 2H), 7.38 (d, J = 8.2 Hz, 2H), 7.24 (d, J = 7.9 Hz, 2H), |
| | 7.09 (d, J = 7.9 Hz, 2H), 4.44-4.24 (m, 3H), 3.71 (d, J = 7.2 Hz, |
| | 1H), 3.65 (d, J = 7.3 Hz, 1H), 2.57 (s, 3H), 2.44 (d, J = 7.1 |
| | Hz, 2H), 1.82 (dq, J = 13.7, 6.8 Hz, 1H), 1.43 |
| | (s, 3H), 1.24 (s, 3H), 0.88 (d, J = 6.6 Hz, 6H). |
| 309b | LCMS (ESI) [M + H]⁺: 529.4 |
| | ¹H NMR (400 MHz, CD₃OD) δ |
| | 8.10-8.00 (m, 1H), 7.85 (d, J = 7.5 Hz, 1H), 7.75 (d, J = 8.0 |
| | Hz, 2H), 7.38 (d, J = 8.2 Hz, 2H), 7.24 (d, J = |
| | 7.8 Hz, 2H), 7.09 (d, J = 8.1 Hz, 2H), 4.42-4.24 |
| | (m, 3H), 3.71 (d, J = 7.2 Hz, 1H), 3.65 (d, J = |
| | 7.3 Hz, 1H), 2.58 (s, 3H), 2.44 (d, J = 7.2 Hz, |
| | 2H), 1.82 (dp, J = 13.8, 6.9 Hz, 1H), 1.43 (s, 3H), |
| | 1.24 (s, 3H), 0.88 (d, J = 6.6 Hz, 6H). |

### Example 310: (S)-4-(5-fluoro-6-(1-((4-isopropylphenyl)carbamoyl)-3,3-dimethylazetidine-2-carboxamido)pyridin-3-yl)benzoic acid and (R)-4-(5-fluoro-6-(1-((4-isopropylphenyl)carbamoyl)-3,3-dimethylazetidine-2-carboxamido)pyridin-3-yl)benzoic acid

### Step 1: 3,3-dimethyl-1-picolinoylazetidine-2-carboxamide

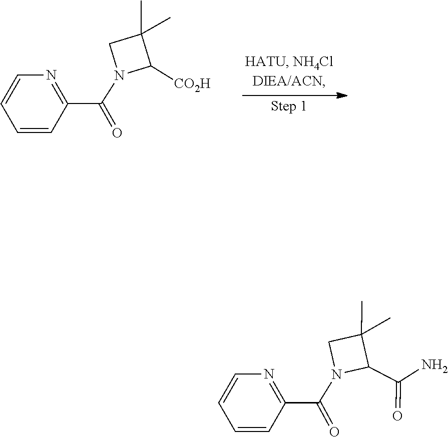 [SMILES: CC1(C)CN(C(=O)c2ccccn2)C1C(=O)O ; CC1(C)CN(C(=O)c2ccccn2)C1C(N)=O]

A solution of 3,3-dimethyl-1-picolinoylazetidine-2-carboxylic acid (1.00 g, 4.27 mmol), DIEA (3.31 g, 25.6 mmol), ammonium chloride (1.14 g, 21.3 mmol) and HATU (2.43 g, 6.40 mmol) in MeCN (15.0 mL) was stirred at 25° C. for 2 hours. Upon completion, the resultant mixture was concentrated under reduced pressure. The crude residue was directly purified by silica gel column chromatography, eluted with methanol/dichloromethane (1:10), to give 3,3-dimethyl-1-picolinoylazetidine-2-carboxamide (950 mg, 95%) as a yellow oil. LCMS (ESI) [M+H]⁺: 234.3.

### Step 2: N-(5-chloro-3-fluoropyridin-2-yl)-3,3-dimethyl-1-picolinoylazetidine-2-carboxamide

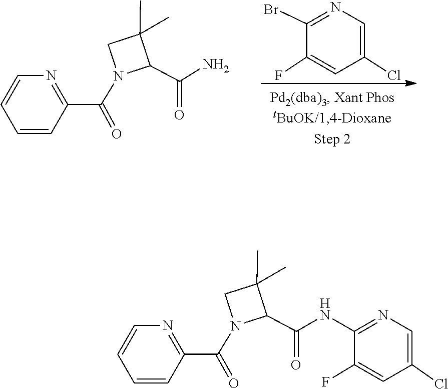 [SMILES: CC1(C)CN(C(=O)c2ccccn2)C1C(N)=O ; Fc1cc(Cl)cnc1Br ; CC1(C)CN(C(=O)c2ccccn2)C1C(=O)Nc1ncc(Cl)cc1F]

A mixture of 3,3-dimethyl-1-(pyridine-2-carbonyl)azetidine-2-carboxamide (500 mg, 2.14 mmol), 2-bromo-5-chloro-3-fluoropyridine (902 mg, 4.29 mmol), Pd₂(dba)₃ (196 mg, 0.214 mmol), XantPhos (248 mg, 0.429 mmol), and potassium tert-butoxide (481 mg, 4.29 mmol) in 1,4-dioxane (7 mL) was heated to 90° C. for 1 hours. Upon completion, the mixture was concentrated under reduced pressure. The crude residue was directly purified by silica gel column chromatography, eluted with petroleum ether/ethyl acetate (3:1) to afford N-(5-chloro-3-fluoropyridin-2-yl)-3,3-dimethyl-1-picolinoylazetidine-2-carboxamide (350 mg, 45%) as a yellow solid. LCMS (ESI) [M+H]⁺: 363.8

### Step 3: methyl 4-(6-(3,3-dimethyl-1-picolinoylazetidine-2-carboxamido)-5-fluoropyridin-3-yl)benzoate

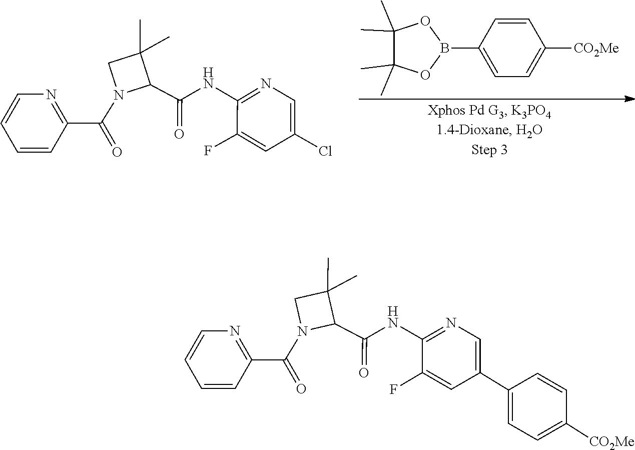 [SMILES: CC1(C)CN(C(=O)c2ccccn2)C1C(=O)Nc1ncc(Cl)cc1F ; COC(=O)c1ccc(B2OC(C)(C)C(C)(C)O2)cc1 ; COC(=O)c1ccc(-c2cnc(NC(=O)C3N(C(=O)c4ccccn4)CC3(C)C)c(F)c2)cc1]

A mixture of N-(5-chloro-3-fluoropyridin-2-yl)-3,3-dimethyl-1-picolinoylazetidine-2-carboxamide (350 mg, 0.965 mmol), methyl 4-(4,4,5,5-tetramethyl-1,3,2-dioxaborolan-2-yl)benzoate (722 mg, 1.93 mmol), XPhos Pd G3 (117 mg, 0.138 mmol) and tribasic potassium phosphate (878 mg, 4.13 mmol) in 1,4-dioxane (5 mL) and water (1 mL) was heated to 80° C. for 1 hour. Upon completion, the reaction mixture was concentrated under reduced pressure. The crude residue was directly purified by silica gel column chromatography, eluted with petroleum ether/ethyl acetate (4:1) to afford methyl 4-(6-(3,3-dimethyl-1-picolinoylazetidine-2-carboxamido)-5-fluoropyridin-3-yl)benzoate (380 mg, 85%) as a light brown solid. LCMS (ESI) [M+H]⁺: 463.5.

### Step 4: Methyl 4-(6-(3,3-dimethylazetidine-2-carboxamido)-5-fluoropyridin-3-yl)benzoate

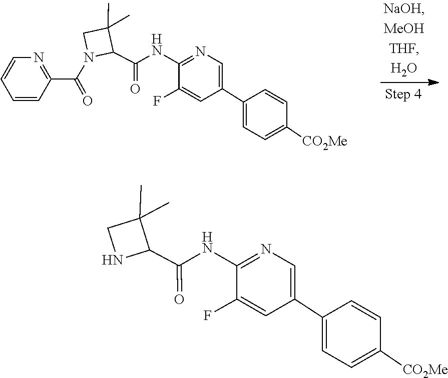 [SMILES: COC(=O)c1ccc(-c2cnc(NC(=O)C3N(C(=O)c4ccccn4)CC3(C)C)c(F)c2)cc1 ; COC(=O)c1ccc(-c2cnc(NC(=O)C3NCC3(C)C)c(F)c2)cc1]

A mixture of methyl 4-(6-(3,3-dimethyl-1-picolinoylazetidine-2-carboxamido)-5-fluoropyridin-3-yl)benzoate (380 mg, 0.822 mmol) and sodium hydroxide (164 mg, 4.11 mmol) in methanol (3 mL), tetrahydrofuran (3 mL), and water (3 mL) was stirred at room temperature for 2 hours. Upon completion, the organic solvent was removed under reduced pressure. The crude residue was directly purified by reverse phase flash chromatography (conditions: column: C18 silica gel; mobile phase A: water (0.1% formic acid); mobile phase B: MeCN; gradient (B %): 0%-100% over 20 min; detector: UV 254/220 nm) to give 4-(6-(3,3-dimethylazetidine-2-carboxamido)-5-fluoropyridin-3-yl)benzoic acid (60 mg, 21%) as an off-white solid. LCMS (ESI) [M+H]⁺: 344.4.

### Step 5: 4-(5-fluoro-6-(1-((4-isopropylphenyl)carbamoyl)-3,3-dimethylazetidine-2-carboxamido)pyridin-3-yl)benzoic acid

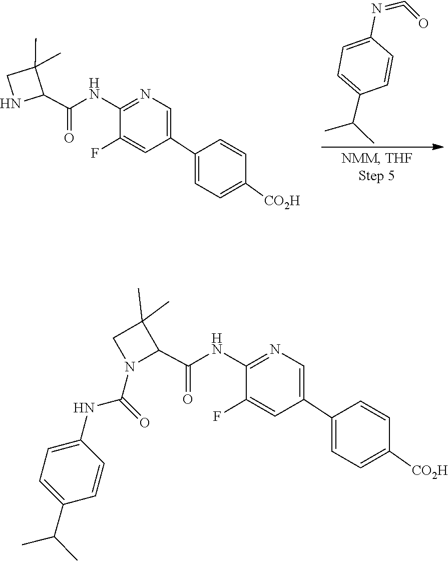 [SMILES: CC1(C)CNC1C(=O)Nc1ncc(-c2ccc(C(=O)O)cc2)cc1F ; CC(C)c1ccc(N=C=O)cc1 ; CC(C)c1ccc(NC(=O)N2CC(C)(C)C2C(=O)Nc2ncc(-c3ccc(C(=O)O)cc3)cc2F)cc1]

A mixture of 4-[6-(3,3-dimethylazetidine-2-amido)-5-fluoropyridin-3-yl]benzoic acid (60 mg, 0.175 mmol), 1-isocyanato-4-isopropylbenzene (56.3 mg, 0.350 mmol) and 4-methylmorpholine (53.0 mg, 0.525 mmol) in tetrahydrofuran (2 mL) was stirred at room temperature for 1 hour. Upon completion, the solvent was removed under reduced pressure. The crude residue was directly purified by reversed phase flash chromatography (conditions: column: C18 silica gel; mobile phase A: water (0.1% formic acid); mobile phase B: MeCN; gradient (B %): 0%-100% over 25 min; detector: UV 254 nm) to give 4-(5-fluoro-6-(1-((4-isopropylphenyl)carbamoyl)-3,3-dimethylazetidine-2-carboxamido)pyridin-3-yl)benzoic acid (26.0 mg, 29%) as an off-white solid. LCMS (ESI) [M+H]⁺: 504.6.

### Step 6: (S)-4-(5-fluoro-6-(1-((4-isopropylphenyl)carbamoyl)-3,3-dimethylazetidine-2-carboxamido)pyridin-3-yl)benzoic acid and (R)-4-(5-fluoro-6-(1-((4-isopropylphenyl)carbamoyl)-3,3-dimethylazetidine-2-carboxamido)pyridin-3-yl)benzoic acid

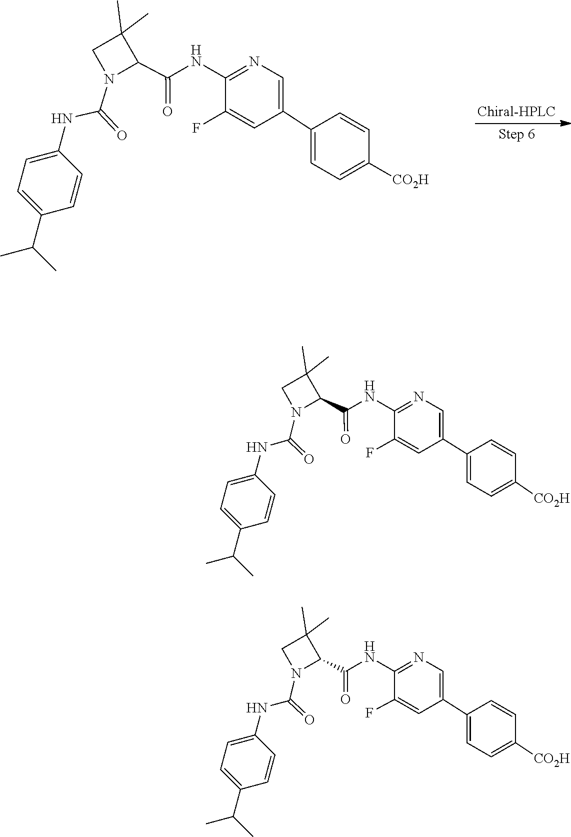 [SMILES: CC(C)c1ccc(NC(=O)N2CC(C)(C)C2C(=O)Nc2ncc(-c3ccc(C(=O)O)cc3)cc2F)cc1 ; CC(C)c1ccc(NC(=O)N2CC(C)(C)[C@H]2C(=O)Nc2ncc(-c3ccc(C(=O)O)cc3)cc2F)cc1 ; CC(C)c1ccc(NC(=O)N2CC(C)(C)[C@@H]2C(=O)Nc2ncc(-c3ccc(C(=O)O)cc3)cc2F)cc1]

4-(5-fluoro-6-(1-((4-isopropylphenyl)carbamoyl)-3,3-dimethylazetidine-2-carboxamido)pyridin-3-yl)benzoic acid (26 mg) was resolved by preparative chiral HPLC (conditions: column: CHIRAL ART Cellulose-SZ, 3*25 cm, 5 μm; mobile phase A: MTBE (0.1% formic acid), mobile phase B: methanol; flow rate: 40 mL/min; gradient (B %): isocratic, 50% B; detector: 220/254 nm; RT1 (min): 6.5; RT2 (min): 16.5) to afford (Compound 310a; Method BL, Peak 1, 0.67 min) (S)-4-(5-fluoro-6-(1-((4-isopropylphenyl)carbamoyl)-3,3-dimethylazetidine-2-carboxamido)pyridin-3-yl)benzoic acid (1^{st} peak, 6.6 mg, 29%) as a white solid, and (Compound 310b; Method BL, Peak 2, 2.02 min) (R)-4-(5-fluoro-6-(1-((4-isopropylphenyl)carbamoyl)-3,3-dimethylazetidine-2-carboxamido)pyridin-3-yl)benzoic acid (2^{nd} peak, 5.7 mg, 25%) as a white solid.

| Cmpd # | Characterization Data |
|---|---|
| 310a | LCMS (ESI) [M + H]⁺: 505.2 |
| | ¹H NMR (300 MHz, CD₃OD) δ |
| | 8.60 (s, 1H), 8.16 (d, J = 8.1 Hz, 2H), 8.06 (d, J = 10.7 |
| | Hz, 1H), 7.83 (d, J = 8.0 Hz, 2H), 7.37 (d, J = 8.2 Hz, 2H), |
| | 7.16 (d, J = 8.2 Hz, 2H), 4.66 (s, 1H), 3.95-3.78 (m, 2H), |
| | 2.98-2.76 (m, 1H), 1.51 (s, 3H), 1.37 (s, 3H), 1.24 |
| | (d, J = 6.9 Hz, 6H). |
| 310b | LCMS (ESI) [M + H]⁺: 505.2 |
| | ¹H NMR (300 MHz, CD₃OD) δ 8.58 |
| | (s, 1H), 8.14 (d, J = 8.1 Hz, 2H), 8.03 (d, J = 10.8, 1.9 Hz, |
| | 1H), 7.80 (d, J = 8.0 Hz, 2H), 7.35 (d, J = 8.2 Hz, |
| | 2H), 7.14 (d, J = 8.2 Hz, 2H), 4.64-4.54 (m, 1H), 3.92-3.76 |
| | (m, 2H), 2.85 (p, J = 6.9 Hz, 1H), 1.48 (s, 3H), 1.35 (s, 3H), |
| | 1.22 (d, J = 6.9 Hz, 6H). |

### Example 311: (S)-5-(4-(3,3-dimethyl-1-((4-(trifluoromethyl)phenyl)carbamoyl)azetidine-2-carboxamido)phenyl)-6-methylpicolinic acid and (R)-5-(4-(3,3-dimethyl-1-((4-(trifluoromethyl)phenyl)carbamoyl)azetidine-2-carboxamido)phenyl)-6-methylpicolinic acid

### Step 1: 5-(4-(3,3-dimethyl-1-((4-(trifluoromethyl)phenyl)carbamoyl)azetidine-2-carboxamido)phenyl)-6-methylpicolinic acid

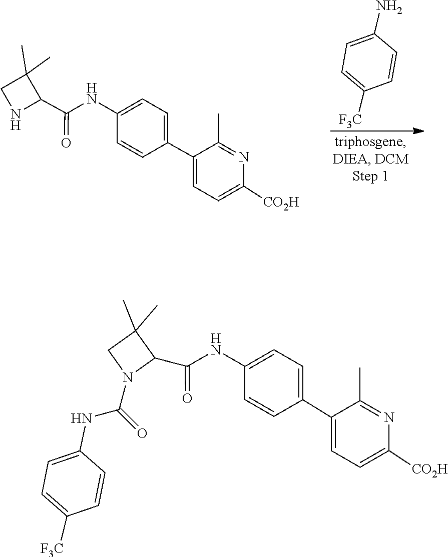 [SMILES: Cc1nc(C(=O)O)ccc1-c1ccc(NC(=O)C2NCC2(C)C)cc1 ; Nc1ccc(C(F)(F)F)cc1 ; Cc1nc(C(=O)O)ccc1-c1ccc(NC(=O)C2N(C(=O)Nc3ccc(C(F)(F)F)cc3)CC2(C)C)cc1]

A solution of 4-(trifluoromethyl)aniline (50.0 mg, 0.310 mmol), 5-(4-(3,3-dimethylazetidine-2-carboxamido)phenyl)-6-methylpicolinic acid (105 mg, 0.310 mmol), DIEA (200 mg, 1.55 mmol), and triphosgene (92 mg, 0.310 mmol) in dichloromethane (2.0 mL) was stirred at 25° C. for 1 hour. Upon completion, the mixture was concentrated under reduced pressure. The crude residue was directly purified by reverse phase flash chromatography (conditions: column: C18 silica gel; mobile phase A: water (0.1% formic acid); mobile phase B: MeCN; gradient (B %): 0%-100% over 25 min; detector: UV 254/220 nm) to give 5-(4-(3,3-dimethyl-1-((4-(trifluoromethyl)phenyl)carbamoyl)azetidine-2-carboxamido)phenyl)-6-methylpicolinic acid (36 mg, 22%) as an off-white solid. LCMS (ESI) [M+H]⁺: 527.1.

### Step 2: (S)-5-(4-(3,3-dimethyl-1-((4-(trifluoromethyl)phenyl)carbamoyl)azetidine-2-carboxamido)phenyl)-6-methylpicolinic acid and (R)-5-(4-(3,3-dimethyl-1-((4-(trifluoromethyl)phenyl)carbamoyl)azetidine-2-carboxamido)phenyl)-6-methylpicolinic acid

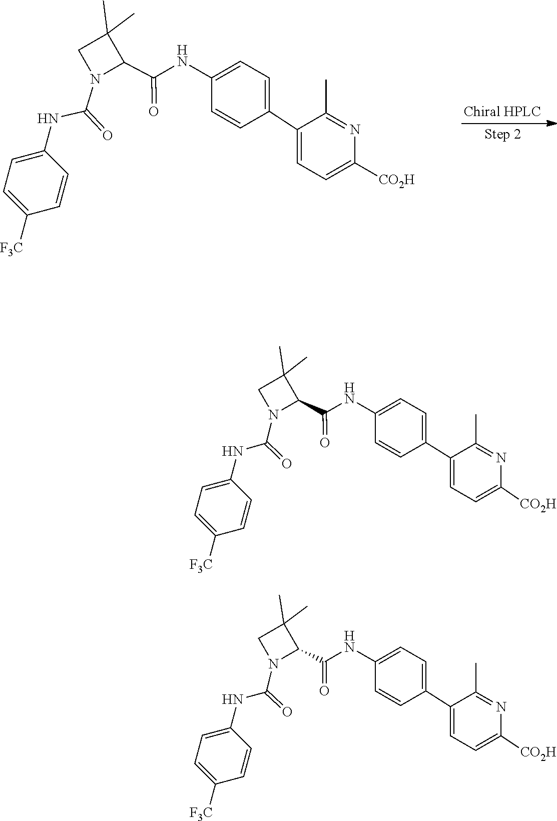 [SMILES: Cc1nc(C(=O)O)ccc1-c1ccc(NC(=O)C2N(C(=O)Nc3ccc(C(F)(F)F)cc3)CC2(C)C)cc1 ; Cc1nc(C(=O)O)ccc1-c1ccc(NC(=O)[C@H]2N(C(=O)Nc3ccc(C(F)(F)F)cc3)CC2(C)C)cc1 ; Cc1nc(C(=O)O)ccc1-c1ccc(NC(=O)[C@@H]2N(C(=O)Nc3ccc(C(F)(F)F)cc3)CC2(C)C)cc1]

5-(4-(3,3-dimethyl-1-((4-(trifluoromethyl)phenyl)carbamoyl)azetidine-2-carboxamido)phenyl)-6-methylpicolinic acid (36 mg) was resolved by preparative chiral HPLC (conditions: column: CHIRAL ART Cellulose-SB, 3*25 cm, 5 μm; mobile phase A: hexane (0.1% formic acid), mobile phase B: ethanol/MeCN (5:1); flow rate: 40 mL/min; gradient (B %): isocratic, 35% B; detector: 220/254 nm; RT1 (min): 6.4; RT2 (min): 16.4) to afford (Compound 311a; Method BW, Peak 1, 1.26 min) (S)-5-(4-(3,3-dimethyl-1-((4-(trifluoromethyl)phenyl)carbamoyl)azetidine-2-carboxamido)phenyl)-6-methylpicolinic acid (1st peak, 12.3 mg, 32%) as a white solid, and (Compound 311b; Method BW, Peak 2, 3.43 min) (R)-5-(4-(3,3-dimethyl-1-((4-(trifluoromethyl)phenyl)carbamoyl)azetidine-2-carboxamido)phenyl)-6-methylpicolinic acid (2^{nd} peak, 13.1 mg, 34%) as a white solid.

| Cmpd # | Characterization Data |
|---|---|
| 311a | LCMS (ESI) [M + H]⁺: 527.2 |
| | ¹H NMR (400 MHz, CD₃OD) δ 8.07 (d, J = 7.9 Hz, 1H), 7.87 (d, J = 7.9 Hz, 1H), 7.78 (d, J = |
| | 8.3 Hz, 2H), 7.69 (d, J = 8.5 Hz, 2H), 7.55 (d, J = 8.7 Hz, 2H), 7.40 (d, J = 8.3 Hz, 2H), 4.53 (s, |
| | 1H), 3.90 (d, J = 7.4 Hz, 1H), 3.82 (d, J = 7.4 Hz, 1H), 2.58 (s, 3H), 1.50 (s, 3H), 1.27 (s, 3H). |
| 311b | LCMS (ESI) [M + H]⁺: 527.2 |
| | ¹H NMR (400 MHz, CD₃OD) δ 8.08 (d, J = 7.9 Hz, 1H), 7.89 (d, J = 7.8 Hz, 1H), 7.78 (d, J = |
| | 8.5 Hz, 2H), 7.69 (d, J = 8.5 Hz, 2H), 7.55 (d, J = 8.6 Hz, 2H), 7.40 (d, J = 8.6 Hz, 2H), 4.53 (s, |
| | 1H), 3.90 (d, J = 7.4 Hz, 1H), 3.82 (d, J = 7.4 Hz, 1H), 2.59 (s, 3H), 1.50 (s, 3H), 1.27 (s, 3H). |

### Example 312: (S)-5-(4-(1-((4-cyclopropyl-3-(trifluoromethyl)phenyl)carbamoyl)-3,3-dimethylazetidine-2-carboxamido)phenyl)-6-methylpicolinic acid and (R)-5-(4-(1-((4-cyclopropyl-3-(trifluoromethyl)phenyl)carbamoyl)-3,3-dimethylazetidine-2-carboxamido)phenyl)-6-methylpicolinic acid

### Step 1: 4-cyclopropyl-3-(trifluoromethyl)aniline

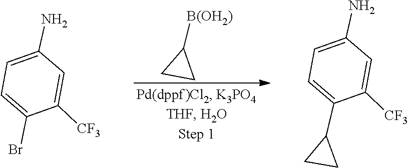

A solution of 4-bromo-3-(trifluoromethyl)aniline (200 mg, 0.833 mmol), cyclopropylboronic acid (143 mg, 1.66 mmol), K₃PO₄ (530 mg, 2.49 mmol) and Pd(dppf)Cl₂ (68.0 mg, 0.083 mmol) in THF (4 mL) and water (1 mL) was heated to 90° C. for 30 minutes. Upon completion, the mixture was concentrated under reduced pressure. The crude residue was directly purified by silica gel column chromatography, eluted with petroleum ether/ethyl acetate (2:1), to afford 4-cyclopropyl-3-(trifluoromethyl)aniline (150 mg, 89%) as a yellow oil. LCMS (ESI) [M+H]⁺: 202.1

### Step 2: 5-(4-(1-((4-cyclopropyl-3-(trifluoromethyl)phenyl)carbamoyl)-3,3-dimethylazetidine-2-carboxamido)phenyl)-6-methylpicolinic acid

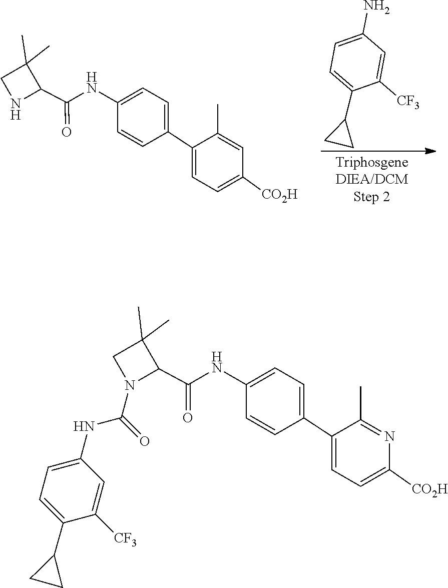 [SMILES: Cc1cc(C(=O)O)ccc1-c1ccc(NC(=O)C2NCC2(C)C)cc1 ; Nc1ccc(C2CC2)c(C(F)(F)F)c1 ; Cc1nc(C(=O)O)ccc1-c1ccc(NC(=O)C2N(C(=O)Nc3ccc(C4CC4)c(C(F)(F)F)c3)CC2(C)C)cc1]

A solution of 4-cyclopropyl-3-(trifluoromethyl)aniline (150 mg, 0.746 mmol), 5-(4-(3,3-dimethylazetidine-2-carboxamido)phenyl)-6-methylpicolinic acid (253 mg, 0.746 mmol), DIEA (481 mg, 3.73 mmol) and triphosgene (88.4 mg, 0.298 mmol) in dichloromethane (2.0 mL) was stirred at 25° C. for 1 hour. Upon completion, the mixture was concentrated under reduced pressure. The crude residue was directly purified by reversed phase flash chromatography (conditions: column: C18 silica gel; mobile phase A: water (0.1% formic acid); mobile phase B: MeCN; gradient (B %): 0%-100% over 25 min; detector: UV 254/220 nm) to give 5-(4-(1-((4-cyclopropyl-3-(trifluoromethyl)phenyl)carbamoyl)-3,3-dimethylazetidine-2-carboxamido)phenyl)-6-methylpicolinic acid (80 mg, 19%) as an off-white solid. LCMS (ESI) [M+H]⁺: 567.2.

### Step 3: (S)-5-(4-(1-((4-cyclopropyl-3-(trifluoromethyl)phenyl)carbamoyl)-3,3-dimethylazetidine-2-carboxamido)phenyl)-6-methylpicolinic acid and (R)-5-(4-(1-((4-cyclopropyl-3-(trifluoromethyl)phenyl)carbamoyl)-3,3-dimethylazetidine-2-carboxamido)phenyl)-6-methylpicolinic acid

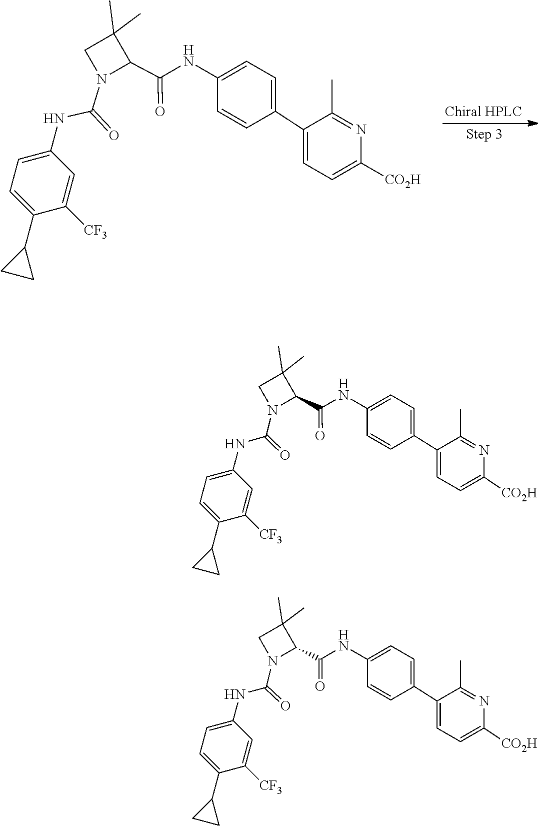 [SMILES: Cc1nc(C(=O)O)ccc1-c1ccc(NC(=O)C2N(C(=O)Nc3ccc(C4CC4)c(C(F)(F)F)c3)CC2(C)C)cc1 ; Cc1nc(C(=O)O)ccc1-c1ccc(NC(=O)[C@H]2N(C(=O)Nc3ccc(C4CC4)c(C(F)(F)F)c3)CC2(C)C)cc1 ; Cc1nc(C(=O)O)ccc1-c1ccc(NC(=O)[C@@H]2N(C(=O)Nc3ccc(C4CC4)c(C(F)(F)F)c3)CC2(C)C)cc1]

5-(4-(1-((4-cyclopropyl-3-(trifluoromethyl)phenyl)carbamoyl)-3,3-dimethylazetidine-2-carboxamido)phenyl)-6-methylpicolinic acid (80 mg) was accomplished by preparative chiral HPLC (conditions: column: CHIRAL ART Cellulose-SZ, 3*25 cm, 5 μm; mobile phase A: hexane (0.1% formic acid), mobile phase B: methanol; flow rate: 40 mL/min; gradient (B %): isocratic, 50% B; detector: 220/254 nm; RT1 (min): 8.3; RT2 (min): 20.5) to afford (Compound 312a; Method BL, Peak 1, 0.65 min) (S)-5-(4-(1-((4-cyclopropyl-3-(trifluoromethyl)phenyl)carbamoyl)-3,3-dimethylazetidine-2-carboxamido)phenyl)-6-methylpicolinic acid (1^{st} peak, 24.0 mg, 30%) as a white solid, and (Compound 312b; Method BL, Peak 2, 2.07 min) (R)-5-(4-(1-((4-cyclopropyl-3-(trifluoromethyl)phenyl)carbamoyl)-3,3-dimethylazetidine-2-carboxamido)phenyl)-6-methylpicolinic acid (2^{nd} peak, 26.3 mg, 33%) as a white solid.

| Cmpd # | Characterization Data |
|---|---|
| 312a | LCMS (ESI) [M + H]⁺: 567.2 |
| | ¹H NMR (400 MHz, CD₃OD) δ 8.06 (d, J = 8.0 Hz, 1H), 7.88-7.80 (m, 2H), 7.77 (d, J = 8.1 |
| | Hz, 2H), 7.61 (dd, J = 8.6, 2.6 Hz, 1H), 7.41-7.33 (m, 2H), 7.08-6.97 (m, 1H), 4.56-4.49 |
| | (m, 1H), 3.87 (d, J = 7.3 Hz, 1H), 3.80 (d, J = 7.3 Hz, 1H), 2.63-2.49 (m, 3H), 2.10 (s, 1H), |
| | 1.48 (s, 3H), 1.26 (d, J = 2.0 Hz, 3H), 1.02-0.92 (m, 2H), 0.78-0.64 (m, 2H). |
| 312b | LCMS (ESI) [M + H]⁺: 567.2 |
| | ¹H NMR (400 MHz, CD₃OD) δ 8.06 (d, J = 7.9 Hz, 1H), 7.88-7.80 (m, 2H), 7.77 (d, J = 7.8 |
| | Hz, 2H), 7.62 (dd, J = 8.1, 3.7 Hz, 1H), 7.38 (t, J = 6.9 Hz, 2H), 7.07-6.99 (m, 1H), 4.51 (s, |
| | 1H), 3.87 (d, J = 7.3 Hz, 1H), 3.79 (d, J = 7.3 Hz, 1H), 2.57 (d, J = 4.5 Hz, 3H), 2.10 (s, 1H), |
| | 1.48 (s, 3H), 1.26 (s, 3H), 1.01-0.93 (m, 2H), 0.77-0.67 (m, 2H). |

### Example 313: (S)-5-(4-(1-((2-fluoro-4-isopropylbenzyl)carbamoyl)-3,3-dimethylazetidine-2-carboxamido)phenyl)-6-methylpicolinic acid and (R)-5-(4-(1-((2-fluoro-4-isopropylbenzyl)carbamoyl)-3,3-dimethylazetidine-2-carboxamido)phenyl)-6-methylpicolinic acid

### Step 1: (2-fluoro-4-(prop-1-en-2-yl)phenyl)methanamine

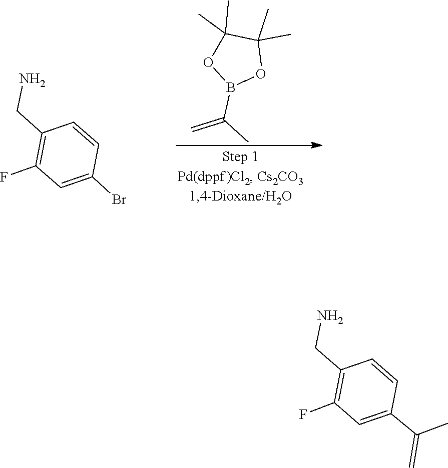

A solution of (4-bromo-2-fluorophenyl)methanamine (1.00 g, 4.90 mmol), Pd(dppf)Cl₂ (0.360 g, 0.490 mmol), 4,4,5,5-tetramethyl-2-(prop-1-en-2-yl)-1,3,2-dioxaborolane (1.65 g, 9.80 mmol) and cesium carbonate (4.79 g, 14.7 mmol) in 1,4-dioxane (20 mL) and water (1 mL) was heated to 100° C. for 1 hour. Upon completion, the mixture was diluted with water (60 mL) and extracted with ethyl acetate (3×80 mL). The combined organic layers were concentrated under reduced pressure. The crude residue was purified by silica gel column chromatography, eluted with petroleum ether/ethyl acetate (1:4) to afford (2-fluoro-4-(prop-1-en-2-yl)phenyl)methanamine (660 mg, 82%) as a brown oil. LCMS (ESI) [M+H]⁺: 166.1.

### Step 2: (2-fluoro-4-isopropylphenyl)methanamine

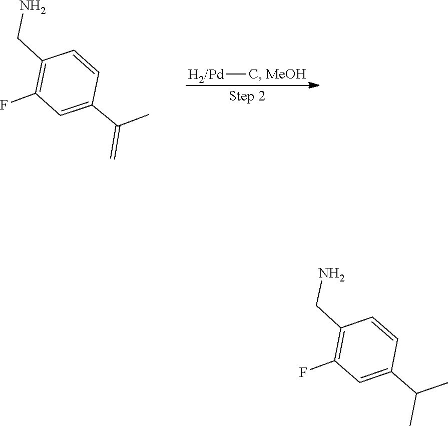

A solution of (2-fluoro-4-(prop-1-en-2-yl)phenyl)methanamine (660 mg, 3.99 mmol) and 10% palladium on carbon (425 mg) in methanol (20 mL) was stirred under an atmosphere (1 atm) of hydrogen at room temperature for 1 hour. Upon completion, the mixture was filtered through celite. The filtrate was concentrated under reduced pressure to afford 1-(2-fluoro-4-isopropylphenyl)methanamine (640 mg, crude) as a tan oil, which was used directly in the next step without further purification. LCMS (ESI) [M+H]⁺: 168.1

### Step 3: 5-(4-(1-((2-fluoro-4-isopropylbenzyl)carbamoyl)-3,3-dimethylazetidine-2-carboxamido)phenyl)-6-methylpicolinic acid

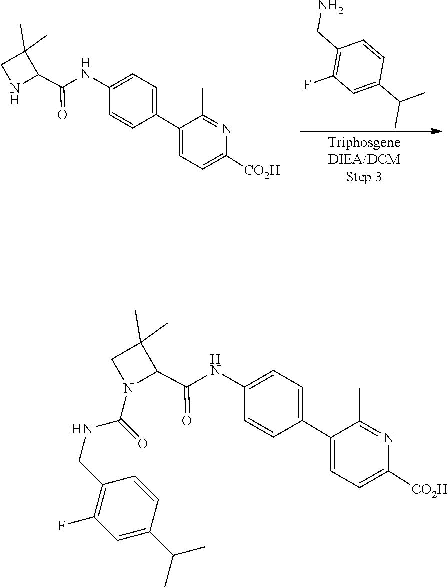 [SMILES: Cc1nc(C(=O)O)ccc1-c1ccc(NC(=O)C2NCC2(C)C)cc1 ; CC(C)c1ccc(CN)c(F)c1 ; Cc1nc(C(=O)O)ccc1-c1ccc(NC(=O)C2N(C(=O)NCc3ccc(C(C)C)cc3F)CC2(C)C)cc1]

A solution of 5-(4-(3,3-dimethylazetidine-2-carboxamido)phenyl)-6-methylpicolinic acid (81.2 mg, 0.239 mmol), 1-(2-fluoro-4-isopropylphenyl)methanamine (200 mg, 1.19 mmol), DIEA (309 mg, 2.39 mmol) and triphosgene (70.9 mg, 0.239 mmol) in dichloromethane (5.0 mL) was stirred at 25° C. for 1 hour. Upon completion, the mixture was concentrated under reduced pressure. The crude residue was directly purified by preparative HPLC (conditions: column: Xselect CSH Prep C18, 30*150 mm 5 μm; mobile phase A: water (0.1% formic acid), mobile phase B: MeCN; flow rate: 60 mL/min; gradient (B %): 31%-44% over 12 min; detector: 254/220 nm; RT1 (min): 11.5 min) to afford 5-(4-(1-((2-fluoro-4-isopropylbenzyl)carbamoyl)-3,3-dimethylazetidine-2-carboxamido)phenyl)-6-methylpicolinic acid (60 mg, 48%) as an off-white solid. LCMS (ESI) [M+H]⁺: 533.2.

### Step 4: (S)-5-(4-(1-((2-fluoro-4-isopropylbenzyl)carbamoyl)-3,3-dimethylazetidine-2-carboxamido)phenyl)-6-methylpicolinic acid and (R)-5-(4-(1-((2-fluoro-4-isopropylbenzyl)carbamoyl)-3,3-dimethylazetidine-2-carboxamido)phenyl)-6-methylpicolinic acid

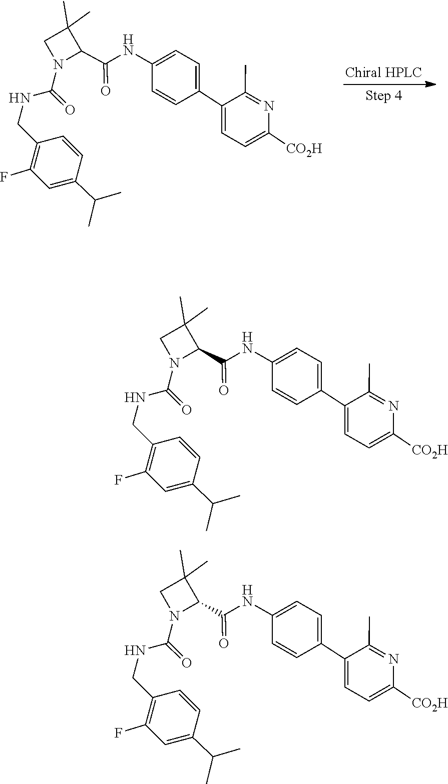

5-(4-(1-((2-fluoro-4-isopropylbenzyl)carbamoyl)-3,3-dimethylazetidine-2-carboxamido)phenyl)-6-methylpicolinic acid (60 mg) was resolved by preparative chiral HPLC (conditions: column: CHIRAL ART Cellulose-SB, 3*25 cm, 5 μm; mobile phase A: hexane (0.1% FA), mobile phase B: ethanol; flow rate: 40 mL/min; gradient (B %): isocratic, 50% B; detector: 220/254 nm; RT1 (min): 9.8; RT2 (min): 14.2) to afford (Compound 313a; Method AY, Peak 1, 1.50 min) (S)-5-(4-(1-((2-fluoro-4-isopropylbenzyl)carbamoyl)-3,3-dimethylazetidine-2-carboxamido)phenyl)-6-methylpicolinic acid (1^{st} peak, 27.7 mg, 58%) as a white solid, and (Compound 313b; Method AY, Peak 2, 2.37 min) (R)-5-(4-(1-((2-fluoro-4-isopropylbenzyl)carbamoyl)-3,3-dimethylazetidine-2-carboxamido)phenyl)-6-methylpicolinic acid (2^{nd} peak, 16.0 mg, 33%) as a white solid.

| Cmpd # | Characterization Data |
|---|---|
| 313a | LCMS (ESI) [M + H]⁺: 533.3 |
| | ¹H NMR (400 MHz, CD₃OD) δ 8.04 (d, J = 7.8 Hz, 1H), 7.83 (d, J = 7.6 Hz, 1H), 7.75 (d, J = |
| | 8.2 Hz, 2H), 7.38 (d, J = 8.1 Hz, 2H), 7.33 (t, J = 8.0 Hz, 1H), 7.01 (dd, J = 7.9, 1.7 Hz, 1H), |
| | 6.92 (dd, J = 11.7, 1.7 Hz, 1H), 4.47-4.31 (m, 3H), 3.72 (d, J = 7.3 Hz, 1H), 3.65 (d, J = 7.3 Hz, |
| | 1H), 2.95-2.82 (m, 1H), 2.57 (s, 3H), 1.43 (s, 3H), 1.31-1.11 (m, 9H). |
| 313b | LCMS (ESI) [M + H]⁺: 533.3 |
| | ¹H NMR (400 MHz, CD₃OD) δ 8.02 (dt, J = 12.7, 6.6 Hz, 1H), 7.82 (dt, J = 8.6, 4.8 Hz, 1H), |
| | 7.75 (d, J = 8.1 Hz, 2H), 7.38 (d, J = 8.0 Hz, 2H), 7.33 (t, J = 8.0 Hz, 1H), 7.01 (d, J = 7.8 Hz, |
| | 1H), 6.92 (dd, J = 11.6, 1.7 Hz, 1H), 4.48-4.27 (m, 3H), 3.72 (d, J = 7.3 Hz, 1H), 3.65 (d, J = |
| | 7.2 Hz, 1H), 2.88 (p, J = 6.9 Hz, 1H), 2.56 (s, 3H), 1.43 (s, 3H), 1.34-1.15 (m, 9H). |

### Example 314: 5-(4-((S)-3,3-dimethyl-1-(((1s,3R)-3-(4-(trifluoromethyl)phenyl)cyclobutyl)carbamoyl)azetidine-2-carboxamido)phenyl)-6-methylpicolinic acid, 5-(4-((S)-3,3-dimethyl-1-(((1r,3S)-3-(4-(trifluoromethyl)phenyl)cyclobutyl)carbamoyl)azetidine-2-carboxamido)phenyl)-6-methylpicolinic acid, 5-(4-((R)-3,3-dimethyl-1-(((1s,3S)-3-(4-(trifluoromethyl)phenyl)cyclobutyl)carbamoyl)azetidine-2-carboxamido)phenyl)-6-methylpicolinic acid and 5-(4-((R)-3,3-dimethyl-1-(((1r,3R)-3-(4-(trifluoromethyl)phenyl)cyclobutyl)carbamoyl)azetidine-2-carboxamido)phenyl)-6-methylpicolinic acid

### Step 1: 3-hydroxy-3-(4-(trifluoromethyl)phenyl)cyclobutane-1-carboxylic acid

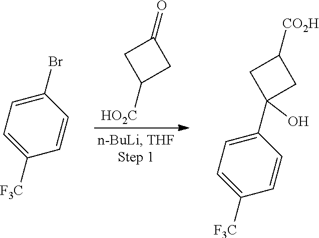 [SMILES: FC(F)(F)c1ccc(Br)cc1 ; O=C1CC(C(=O)O)C1 ; O=C(O)C1CC(O)(c2ccc(C(F)(F)F)cc2)C1]

n-BuLi (10.6 mL, 26.6 mmol, 2.0 M in hexane) was added into

To a solution of 1-bromo-4-(trifluoromethyl)benzene (3.00 g, 13.3 mmol) in THF (30 mL) at −78° C. was added a solution of n-BuLi (10.6 mL, 26.6 mmol, 2.0 M in hexane). After 5 minutes, 3-oxocyclobutane-1-carboxylic acid (2.30 g, 20.0 mmol) was added dropwise. The resultant mixture was warmed to room temperature and stirred overnight. Upon completion, the mixture was quenched with ice water and extracted with ethyl acetate (3×15 mL). The combined organic layers were concentrated under reduced pressure. The crude residue was purified by reversed-phase flash chromatography (conditions: column: C18 silica gel; mobile phase A: water (0.1% formic acid); mobile phase B: MeCN; gradient (B %): 0%-100% over 30 min; detector: UV 200 nm) to afford 3-hydroxy-3-(4-(trifluoromethyl)phenyl)cyclobutane-1-carboxylic acid (1.20 g, 34%) as a white solid. LCMS (ESI) [M−H]: 259.0.

### Step 2: 3-(4-(trifluoromethyl)phenyl)cyclobutane-1-carboxylic acid

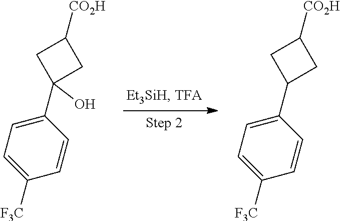 [SMILES: O=C(O)C1CC(O)(c2ccc(C(F)(F)F)cc2)C1 ; O=C(O)C1CC(c2ccc(C(F)(F)F)cc2)C1]

To a solution of 3-hydroxy-3-(4-(trifluoromethyl)phenyl)cyclobutane-1-carboxylic acid (1.20 g, 4.61 mmol) in TFA (7.0 mL) was added triethylsilane (2.0 mL), and the resultant mixture was heated to 90° C. overnight. Upon completion, the mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0%-100% over 30 min; detector: UV 200 nm) to afford 3-(4-(trifluoromethyl)phenyl)cyclobutane-1-carboxylic acid (600 mg, 53%) as a white solid. LCMS (ESI) [M−H]⁻: 243.0

### Step 3: methyl 5-(4-(3,3-dimethyl-1-((3-(4-(trifluoromethyl)phenyl)cyclobutyl)carbamoyl)azetidine-2-carboxamido)phenyl)-6-methylpicolinate

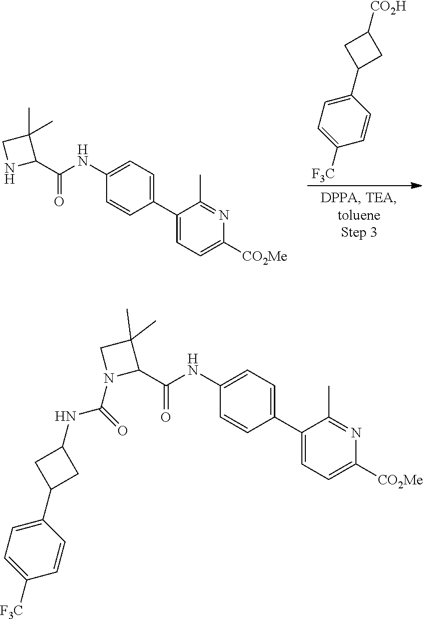 [SMILES: COC(=O)c1ccc(-c2ccc(NC(=O)C3NCC3(C)C)cc2)c(C)n1 ; O=C(O)C1CC(c2ccc(C(F)(F)F)cc2)C1 ; COC(=O)c1ccc(-c2ccc(NC(=O)C3N(C(=O)NC4CC(c5ccc(C(F)(F)F)cc5)C4)CC3(C)C)cc2)c(C)n1]

A solution of 3-(4-(trifluoromethyl)phenyl)cyclobutane-1-carboxylic acid (198 mg, 0.80 mmol), DPPA (343 mg, 1.24 mmol), methyl 5-(4-(3,3-dimethylazetidine-2-carboxamido)phenyl)-6-methylpicolinate (220 mg, 0.620 mmol) and triethylamine (189 mg, 1.86 mmol) in toluene (3.0 mL) was heated to 80° C. for 2 hours. Upon completion, the mixture was concentrated under reduced pressure. The crude residue was directly purified by silica gel column chromatography, eluted with petroleum ether/ethyl acetate (1:1), to afford a crude product (450 mg). The crude product was further purified by reversed-phase flash chromatography (conditions: column: C18 silica gel; mobile phase A: water (0.1% formic acid); mobile phase B: MeCN; gradient (B %): 30%-100% over 20 min; detector: UV 254 nm) to afford methyl 5-(4-(3,3-dimethyl-1-((3-(4-(trifluoromethyl)phenyl)cyclobutyl)carbamoyl)azetidine-2-carboxamido)phenyl)-6-methylpicolinate (150 mg, 40%) as a pale-yellow oil. LCMS (ESI) [M+H]⁺: 595.2.

### Step 4: 5-(4-(3,3-dimethyl-1-((3-(4-(trifluoromethyl)phenyl)cyclobutyl)carbamoyl)azetidine-2-carboxamido)phenyl)-6-methylpicolinic acid

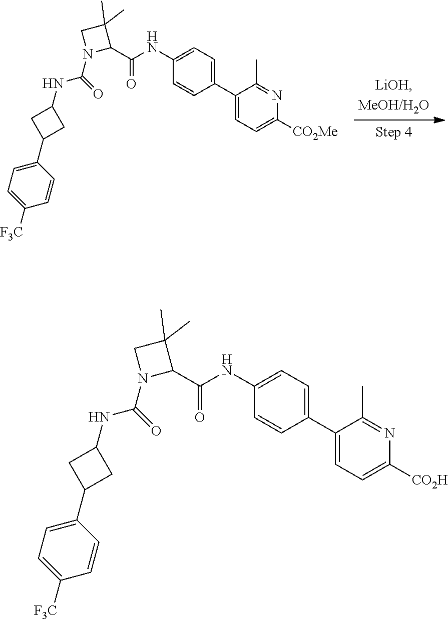 [SMILES: COC(=O)c1ccc(-c2ccc(NC(=O)C3N(C(=O)NC4CC(c5ccc(C(F)(F)F)cc5)C4)CC3(C)C)cc2)c(C)n1 ; Cc1nc(C(=O)O)ccc1-c1ccc(NC(=O)C2N(C(=O)NC3CC(c4ccc(C(F)(F)F)cc4)C3)CC2(C)C)cc1]

A solution of methyl 5-(4-(3,3-dimethyl-1-((3-(4-(trifluoromethyl)phenyl)cyclobutyl)carbamoyl)azetidine-2-carboxamido)phenyl)-6-methylpicolinate (150 mg, 0.25 mmol) and lithium hydroxide monohydrate (212 mg, 5.04 mmol) in methanol (2.0 mL) and water (2.0 mL) was stirred at room temperature for 2 hours. Upon completion, the mixture was concentrated under reduced pressure. The crude residue was purified by reversed-phase flash chromatography (conditions: column: C18 silica gel; mobile phase A: water (0.1% formic acid); mobile phase B: MeCN; gradient (B %): 25% to 100% over 20 min; detector: UV 254 nm) to afford 5-(4-(3,3-dimethyl-1-((3-(4-(trifluoromethyl)phenyl)cyclobutyl)carbamoyl)azetidine-2-carboxamido)phenyl)-6-methylpicolinic acid (103 mg, 70%) as a white solid. LCMS (ESI) [M+H]⁺: 581.2.

### Step 5: 5-(4-((S)-3,3-dimethyl-1-(((1s,3R)-3-(4-(trifluoromethyl)phenyl)cyclobutyl)carbamoyl)azetidine-2-carboxamido)phenyl)-6-methylpicolinic acid, 5-(4-((S)-3,3-dimethyl-1-(((1r,3S)-3-(4-(trifluoromethyl)phenyl)cyclobutyl)carbamoyl)azetidine-2-carboxamido)phenyl)-6-methylpicolinic acid, 5-(4-((R)-3,3-dimethyl-1-(((1s,3S)-3-(4-(trifluoromethyl)phenyl)cyclobutyl)carbamoyl)azetidine-2-carboxamido)phenyl)-6-methylpicolinic acid and 5-(4-((R)-3,3-dimethyl-1-(((1r,3R)-3-(4-(trifluoromethyl)phenyl)cyclobutyl)carbamoyl)azetidine-2-carboxamido)phenyl)-6-methylpicolinic acid

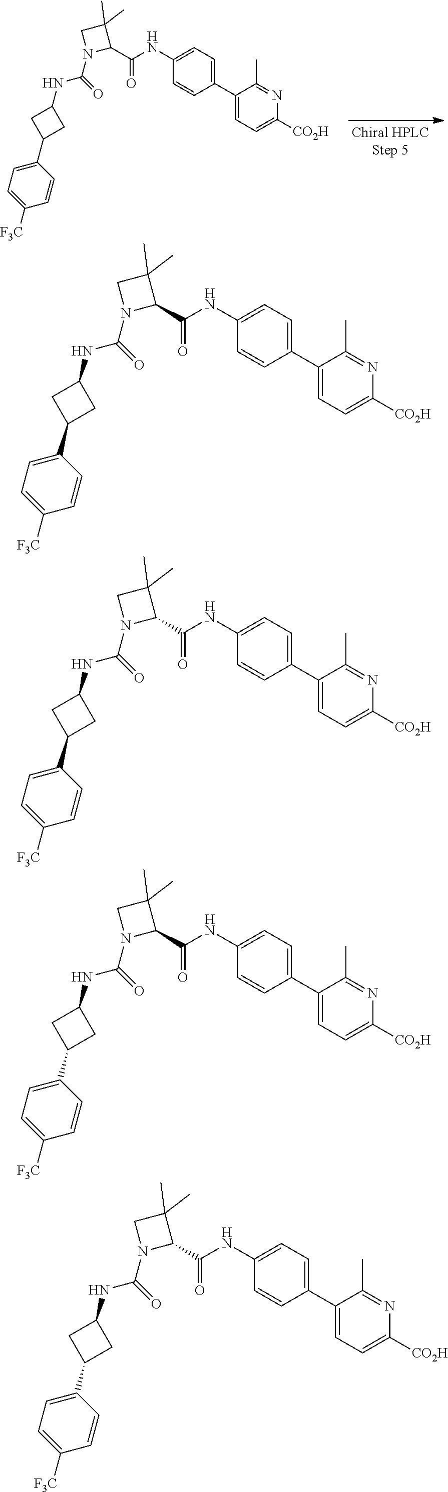 [SMILES: Cc1nc(C(=O)O)ccc1-c1ccc(NC(=O)C2N(C(=O)NC3CC(c4ccc(C(F)(F)F)cc4)C3)CC2(C)C)cc1 ; Cc1nc(C(=O)O)ccc1-c1ccc(NC(=O)[C@H]2N(C(=O)N[C@H]3C[C@@H](c4ccc(C(F)(F)F)cc4)C3)CC2(C)C)cc1 ; Cc1nc(C(=O)O)ccc1-c1ccc(NC(=O)[C@@H]2N(C(=O)N[C@H]3C[C@@H](c4ccc(C(F)(F)F)cc4)C3)CC2(C)C)cc1 ; Cc1nc(C(=O)O)ccc1-c1ccc(NC(=O)[C@H]2N(C(=O)N[C@H]3C[C@H](c4ccc(C(F)(F)F)cc4)C3)CC2(C)C)cc1 ; Cc1nc(C(=O)O)ccc1-c1ccc(NC(=O)[C@@H]2N(C(=O)N[C@H]3C[C@H](c4ccc(C(F)(F)F)cc4)C3)CC2(C)C)cc1]

5-(4-(3,3-dimethyl-1-((3-(4-(trifluoromethyl)phenyl)cyclobutyl)carbamoyl)azetidine-2-carboxamido)phenyl)-6-methylpicolinic acid (103 mg) was resolved by preparative chiral HPLC (conditions: column: CHIRAL ART Cellulose-SZ, 3*25 cm, 5 μm; mobile phase A: MTBE (0.1% formic acid), mobile phase B: methanol; flow rate: 40 mL/min; gradient (B %) 50% B; detector: 220/254 nm; RT1 (min): 6.9; RT2 (min): 8.6; RT3 (min): 22.4; RT4 (min): 30.4) to afford (Compound 314a; Method BL, Peak 1, 0.71 min) 5-(4-((S)-3,3-dimethyl-1-(((1s,3R)-3-(4-(trifluoromethyl)phenyl)cyclobutyl)carbamoyl)azetidine-2-carboxamido)phenyl)-6-methylpicolinic acid (1^{st} peak, 24.8 mg, 24%) as a white solid, (Compound 314b; Method BL, Peak 2, 0.90 min) 5-(4-((S)-3,3-dimethyl-1-(((1r,3S)-3-(4-(trifluoromethyl)phenyl)cyclobutyl)carbamoyl)azetidine-2-carboxamido)phenyl)-6-methylpicolinic acid (2^{nd} peak, 26.4 mg, 25%) as a white solid, (Compound 314c; Method BL, Peak 3, 2.52 min) 5-(4-((R)-3,3-dimethyl-1-(((1s,3S)-3-(4-(trifluoromethyl)phenyl)cyclobutyl)carbamoyl)azetidine-2-carboxamido)phenyl)-6-methylpicolinic acid (3^{rd} peak, 20.9 mg, 20%) as a white solid and (Compound 314d; Method BL, Peak 4, 3.15 min) 5-(4-((R)-3,3-dimethyl-1-(((1r,3R)-3-(4-(trifluoromethyl)phenyl)cyclobutyl)carbamoyl)azetidine-2-carboxamido)phenyl)-6-methylpicolinic acid (4^{th} peak, 16.2 mg, 15%) as a white solid.

| Cmpd # | Characterization Data |
|---|---|
| 314a | LCMS (ESI) [M + H]⁺: 581.3 |
| | ¹H NMR (400 MHz, CD₃OD) δ 8.06 (d, J = 7.8 Hz, 1H), 7.85 (d, J = 7.9 Hz, 1H), 7.76 (d, J = |
| | 8.2 Hz, 2H), 7.60 (d, J = 8.2 Hz, 2H), 7.49 (d, J = 8.1 Hz, 2H), 7.38 (d, J = 8.1 Hz, 2H), 4.40 |
| | (d, J = 3.9 Hz, 2H), 3.74 (d, J = 7.3 Hz, 1H), 3.67 (d, J = 7.2 Hz, 2H), 2.55 (d, J = 13.5 Hz, |
| | 7H), 1.44 (s, 3H), 1.25 (s, 3H). |
| 314b | LCMS (ESI) [M + H]⁺: 581.3 |
| | ¹H NMR (400 MHz, CD₃OD) δ 8.06 (dd, J = 7.9, 4.2 Hz, 1H), 7.86 (dd, J = 7.9, 4.3 Hz, 1H), |
| | 7.76 (dd, J = 8.5, 1.7 Hz, 2H), 7.58 (d, J = 8.1 Hz, 2H), 7.45 (d, J = 8.1 Hz, 2H), 7.39 (d, J = |
| | 8.3 Hz, 2H), 4.38 (d, J = 1.2 Hz, 1H), 4.33-4.20 (m, 1H), 3.71 (d, J = 7.3 Hz, 1H), 3.64 (d, J = |
| | 7.3 Hz, 1H), 3.28-3.18 (m, 1H), 2.83-2.68 (m, 2H), 2.58 (s, 3H), 2.22-2.08 (m, 2H), 1.43 |
| | (s, 3H), 1.24 (s, 3H). |
| 314c | LCMS (ESI) [M + H]⁺: 581.2 |
| | ¹H NMR (400 MHz, CD₃OD) δ 8.06 (d, J = 7.9 Hz, 1H), 7.86 (d, J = 7.8 Hz, 1H), 7.76 (d, J = |
| | 8.2 Hz, 2H), 7.58 (d, J = 8.1 Hz, 2H), 7.45 (d, J = 8.0 Hz, 2H), 7.39 (d, J = 8.3 Hz, 2H), 4.38 |
| | (s, 1H), 4.33-4.22 (m, 1H), 3.71 (d, J = 7.2 Hz, 1H), 3.64 (d, J = 7.3 Hz, 1H), 3.27-3.21 (m, |
| | 1H), 2.81-2.70 (m, 2H), 2.58 (s, 3H), 2.23-2.07 (m, 2H), 1.43 (s, 3H), 1.24 (s, 3H). |
| 314d | LCMS (ESI) [M + H]⁺: 581.2 |
| | ¹H NMR (400 MHz, CD₃OD) δ 8.06 (d, J = 7.8 Hz, 1H), 7.85 (d, J = 7.8 Hz, 1H), 7.76 (d, J = |
| | 8.2 Hz, 2H), 7.60 (d, J = 8.1 Hz, 2H), 7.49 (d, J = 8.1 Hz, 2H), 7.38 (d, J = 8.1 Hz, 2H), 4.44- |
| | 4.35 (m, 2H), 3.74 (d, J = 7.3 Hz, 1H), 3.69-3.59 (m, 2H), 2.63-2.47 (m, 7H), 1.44 (s, 3H), |
| | 1.25 (s, 3H). |

### Example 315: (R)-2-(5-(1H-[1,2,3]triazolo[4,5-b]pyridin-5-yl)-3-fluoropyridin-2-yl)-N-(2,2-difluoro-[1,3]dioxolo[4,5-b]pyridin-6-yl)pyrrolidine-1-carboxamide

### Step 1: (R)-(6-(1-(tert-butoxycarbonyl)pyrrolidin-2-yl)-5-fluoropyridin-3-yl)boronic acid

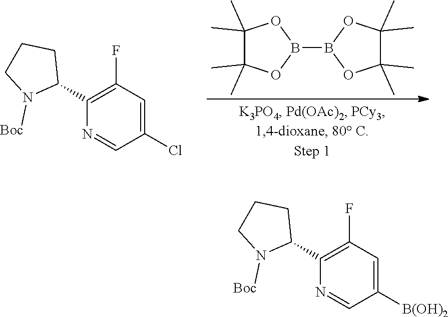

A solution of tert-butyl (R)-2-(5-chloro-3-fluoropyridin-2-yl)pyrrolidine-1-carboxylate (200 mg, 0.666 mmol), 4,4,4′,4′,5,5,5′,5′-octamethyl-2,2′-bi(1,3,2-dioxaborolane) (1.69 g, 6.65 mmol), K₃PO₄ (423 mg, 1.99 mmol), Pd(OAc)₂ (15.0 mg, 60 μmol) and PCy₃ (37.0 mg, 130 μmol) in 1,4-dioxane (20 mL) was heated to 80° C. overnight. Upon completion, the mixture was cooled to room temperature and concentrated under reduced pressure. The crude residue was directly purified by reverse phase flash chromatography (conditions: column: C18 silica gel; mobile phase A: water (0.1% formic acid), mobile phase B: MeCN; gradient (B %): 5%-95% over 15 min; detector: UV 254 nm) to afford (R)-(6-(1-(tert-butoxycarbonyl)pyrrolidin-2-yl)-5-fluoropyridin-3-yl)boronic acid (116 mg, 56%) as a white solid. LCMS (ESI) [M+H]⁺: 311.1.

### Step 2: tert-butyl (R)-2-(3-fluoro-5-(1-((2-(trimethylsilyl)ethoxy)methyl)-1H-[1,2,3]triazolo[4,5-b]pyridin-5-yl)pyridin-2-yl)pyrrolidine-1-carboxylate

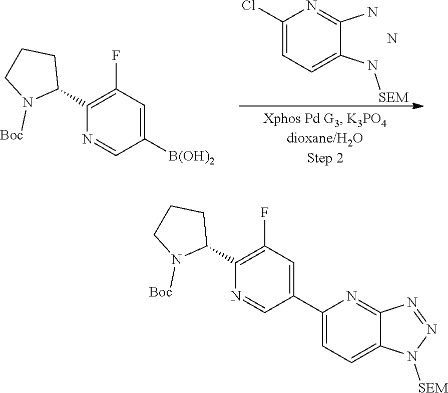

A solution of (R)-(6-(1-(tert-butoxycarbonyl)pyrrolidin-2-yl)-5-fluoropyridin-3-yl)boronic acid (110 mg, 350 μmol), 5-chloro-1-((2-(trimethylsilyl)ethoxy)methyl)-1H-[1,2,3]triazolo[4,5-b]pyridine (121 mg, 420 μmol), Xphos Pd G3 (30 mg, 30 μmol), K₃PO₄ (151 mg, 710 μmol) and water (1.0 mL) in 1,4-dioxane (5.0 mL) was stirred at 80° C. overnight. Upon completion, the mixture was cooled to room temperature and concentrated under reduced pressure. The crude residue was directly purified by silica gel column chromatography, eluted with petroleum ether/ethyl acetate (1:1) to afford tert-butyl (R)-2-(3-fluoro-5-(1-((2-(trimethylsilyl)ethoxy)methyl)-1H-[1,2,3]triazolo[4,5-b]pyridin-5-yl)pyridin-2-yl)pyrrolidine-1-carboxylate (181 mg, 31%) as a yellow solid. LCMS (ESI) [M+H]⁺: 515.2

### Step 3: (R)-5-(5-fluoro-6-(pyrrolidin-2-yl)pyridin-3-yl)-1H-[1,2,3]triazolo[4,5-b]pyridine

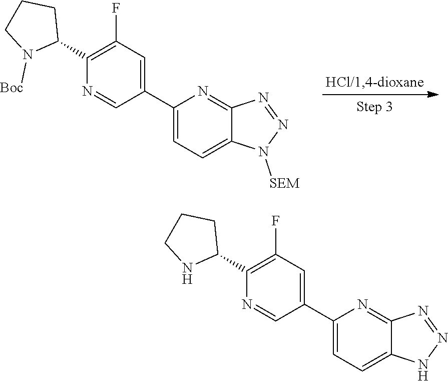

To a mixture of tert-butyl (R)-2-(3-fluoro-5-(1-((2-(trimethylsilyl)ethoxy)methyl)-1H-[1,2,3]triazolo[4,5-b]pyridin-5-yl)pyridin-2-yl)pyrrolidine-1-carboxylate (176 mg, 0.340 mmol) in 1,4-dioxane (2.0 mL) was added a solution of hydrochloric acid (2.00 mL, 8.00 mmol, 4.0 M in 1,4-dioxane) at room temperature. After 1 hour, the mixture was concentrated under reduced pressure to afford crude (R)-5-(5-fluoro-6-(pyrrolidin-2-yl)pyridin-3-yl)-1H-[1,2,3]triazolo[4,5-b]pyridine (120 mg, crude), which was used directly in next step without further purification. LCMS (ESI) [M+H]⁺: 285.1.

### Step 4: (R)-2-(5-(1H-[1,2,3]triazolo[4,5-b]pyridin-5-yl)-3-fluoropyridin-2-yl)-N-(2,2-difluoro-[1,3]dioxolo[4,5-b]pyridin-6-yl)pyrrolidine-1-carboxamide

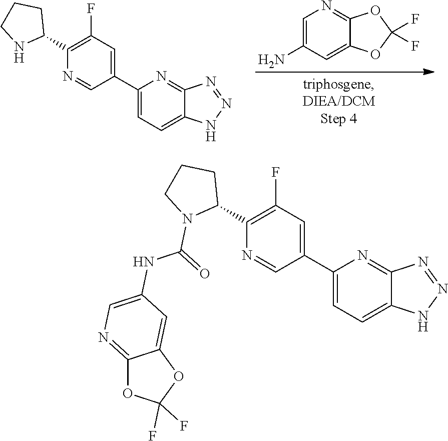

To a solution of triphosgene (12.4 mg, 40 μmol) in dichloromethane (1.0 mL) at 0° C. was added DIEA (90.4 mg, 0.700 mmol) and 2,2-difluoro-[1,3]dioxolo[4,5-b]pyridin-6-amine (21.9 mg, 0.120 mmol) at 0° C. After 30 minutes, (R)-5-chloro-3-fluoro-2-(pyrrolidin-2-yl)pyridine (50.0 mg, 0.140 mmol) was added. The resultant mixture was warmed to room temperature and stirred for 1 hour. Upon completion, the mixture was concentrated under reduced pressure. The crude residue was directly purified by preparative HPLC (conditions: column: Xselect CSH Prep C18 30*150 mm 5 μm; mobile phase A: water (0.1% formic acid), mobile phase B: MeCN; flow rate: 60 mL/min; gradient (B %): 27%-40% B over 17 min; detector: 254/220 nm; RT1 (min): 12.6 min) to afford (R)-2-(5-(1H-[1,2,3]triazolo[4,5-b]pyridin-5-yl)-3-fluoropyridin-2-yl)-N-(2,2-difluoro-[1,3]dioxolo[4,5-b]pyridin-6-yl)pyrrolidine-1-carboxamide (Compound 315) (10.2 mg, 30%) as a white solid.

| Compound# | Characterization Data |
|---|---|
| 315 | LCMS (ESI) [M + H]⁺: 485.2 |
| | ¹H NMR (400 MHz, DMSO-d₆) δ 9.12 (s, 1H), 8.74 (s, 1H), 8.52 (d, J = 8.8 Hz, 1H), 8.35 (d, J = |
| | 11.6 Hz, 1H), 8.14-8.11 (m, 1H), 8.01 (s, 2H), 5.41-5.33 (m, 1H), 3.84-3.75 (m, 1H), |
| | 3.72-3.63 (m, 1H), 2.45-2.31 (m, 1H), 2.19-1.95 (m, 2H), 1.94-1.88 (m, 1H). |

### Example 316: (R)-3-(5-(1H-[1,2,3]triazolo[4,5-b]pyridin-5-yl)-3-fluoropyridin-2-yl)-N-(2,2-difluorobenzo[d][1,3]dioxol-5-yl)morpholine-4-carboxamide and (S)-3-(5-(1H-[1,2,3]triazolo[4,5-b]pyridin-5-yl)-3-fluoropyridin-2-yl)-N-(2,2-difluorobenzo[d][1,3]dioxol-5-yl)morpholine-4-carboxamide

### Step 1: tert-butyl 3-(5-chloro-3-fluoropyridin-2-yl)morpholine-4-carboxylate

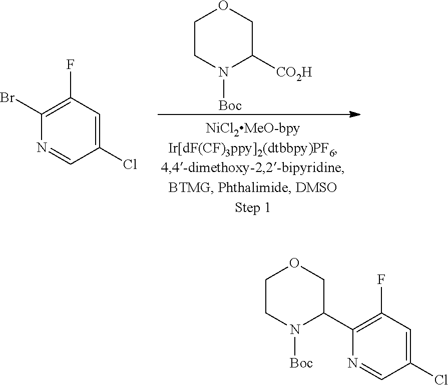 [SMILES: Fc1cc(Cl)cnc1Br ; CC(C)(C)OC(=O)N1CCOCC1C(=O)O ; CC(C)(C)OC(=O)N1CCOCC1c1ncc(Cl)cc1F]

A solution of 2-bromo-5-chloro-3-fluoropyridine (3.00 g, 14.2 mmol), 4-(tert-butoxycarbonyl)morpholine-3-carboxylic acid (4.95 g, 21.3 mmol), dichloronickel;1,2-dimethoxyethane (313 mg, 1.42 mmol), 4,4′-dimethoxy-2,2′-bipyridine (369 mg, 1.71 mmol), N″-tert-butyl-N,N,N′,N′-tetramethylguanidine (3.66 g, 21.3 mmol), Ir[dF(CF₃)ppy]₂(dtbpy))PF₆ (319 mg, 0.285 mmol) and 2,3-dihydro-1H-isoindole-1,3-dione (2.10 g, 14.2 mmol) in DMSO (90 mL) at room temperature was stirred under 450 nm irradiation for 24 hours. Upon completion, the mixture was diluted with water (100 mL) and extracted with EtOAc (3×150 mL). The combined organic layers were washed with brine, dried over anhydrous Na₂SO₄, filtered, and concentrated under reduced pressure. The crude residue was purified by reversed-phase flash chromatography (conditions: column: C18 silica gel; mobile phase A: water (0.1% formic acid); mobile phase B: MeCN; gradient (B %): 25%-100% over 20 min; detector: UV 254 nm) to afford tert-butyl 3-(5-chloro-3-fluoropyridin-2-yl)morpholine-4-carboxylate (1.80 g, 39%) as a pale-yellow solid. LCMS (ESI) [M+H]⁺: 317.0.

### Step 2: 3-(5-chloro-3-fluoropyridin-2-yl)morpholine

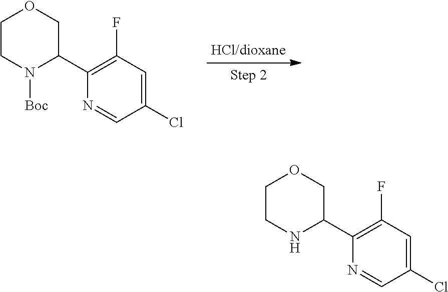 [SMILES: CC(C)(C)OC(=O)N1CCOCC1c1ncc(Cl)cc1F ; Fc1cc(Cl)cnc1C1COCCN1]

To a mixture of tert-butyl 3-(5-chloro-3-fluoropyridin-2-yl)morpholine-4-carboxylate (1.75 g, 5.52 mmol) in 1,4-dioxane (10.0 mL) was added a solution of hydrochloric acid (10 mL, 40.0 mmol, 4.0 M in 1,4-dioxane) and the resultant mixture was stirred at room temperature for 2 hours. Upon completion, the mixture was concentrated under reduced pressure. The crude residue was directly purified by reversed-phase flash chromatography (conditions: column: C18 silica gel; mobile phase A: water (0.1% ammonium bicarbonate); mobile phase B: MeCN; gradient (B %): 0%-100% over 20 min; detector: UV 254 nm) to afford 3-(5-chloro-3-fluoropyridin-2-yl)morpholine (1.10 g, 91%) as a white solid. LCMS (ESI) [M+H]⁺: 217.1.

### Step 3: 3-(5-chloro-3-fluoropyridin-2-yl)-N-(2,2-difluorobenzo[d][1,3]dioxol-5-yl)morpholine-4-carboxamide

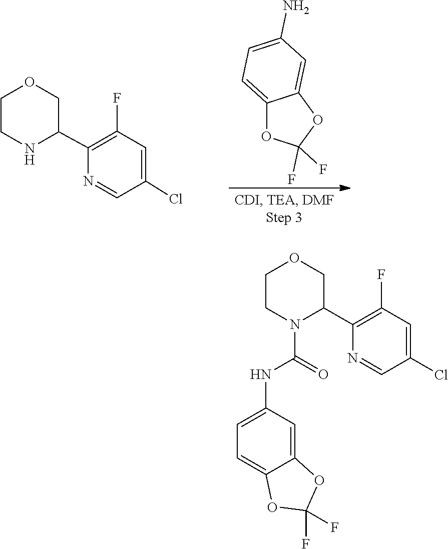 [SMILES: Fc1cc(Cl)cnc1C1COCCN1 ; Nc1ccc2c(c1)OC(F)(F)O2 ; O=C(Nc1ccc2c(c1)OC(F)(F)O2)N1CCOCC1c1ncc(Cl)cc1F]

A mixture of 2,2-difluoro-1,3-benzodioxol-5-amine (639 mg, 3.69 mmol), CDI (598 mg, 3.69 mmol), triethylamine (373 mg, 3.69 mmol), and 3-(5-chloro-3-fluoropyridin-2-yl)morpholine (200 mg, 0.923 mmol) in DMF (10.0 mL) was heated to 60° C. for 2 hours. Upon completion, the mixture was diluted with water (50 mL) and extracted with ethyl acetate (3×50 mL). The combined organic layers were washed with brine, dried over anhydrous Na₂SO₄, filtered, and concentrated under reduced pressure. The crude residue was purified by reversed-phase flash chromatography (conditions: column: C18 silica gel; mobile phase A: water (0.1% formic acid); mobile phase B: MeCN; gradient (B %): 0%-100% over 20 min; detector: UV 254 nm) to afford 3-(5-chloro-3-fluoropyridin-2-yl)-N-(2,2-difluorobenzo[d][1,3]dioxol-5-yl)morpholine-4-carboxamide (170 mg, 44%) as a pale-yellow solid. LCMS (ESI) [M+H]⁺: 416.1.

### Step 4: (6-(4-((2,2-difluorobenzo[d][1,3]dioxol-5-yl)carbamoyl)morpholin-3-yl)-5-fluoropyridin-3-yl)boronic acid

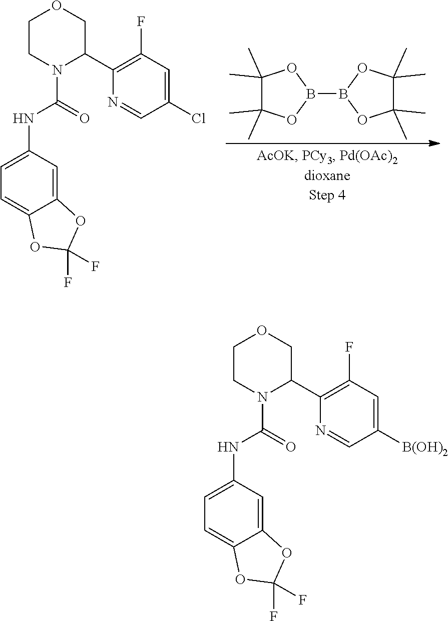

A solution of 3-(5-chloro-3-fluoropyridin-2-yl)-N-(2,2-difluorobenzo[d][1,3]dioxol-5-yl)morpholine-4-carboxamide (150 mg, 0.361 mmol), 4,4,4′,4′,5,5,5′,5′-octamethyl-2,2′-bi(1,3,2-dioxaborolane) (916 mg, 3.61 mmol), potassium acetate (106 mg, 1.08 mmol), Pd(OAc)₂ (8.00 mg, 36 μmol) and PCy₃ (20.0 mg, 72 μmol) in 1,4-dioxane (5.0 mL) was heated to 80° C. overnight. Upon completion, the mixture was cooled to room temperature and concentrated under reduced pressure. The crude residue was directly purified by reversed-phase flash chromatography (conditions: column: C18 silica gel; mobile phase A: water (0.1% formic acid); mobile phase B: MeCN; gradient (B %): 0%-100% over 25 min; detector: UV 254 nm) to afford (6-(4-((2,2-difluorobenzo[d][1,3]dioxol-5-yl)carbamoyl)morpholin-3-yl)-5-fluoropyridin-3-yl)boronic acid (50 mg, 32%) as a white solid. LCMS (ESI) [M+H]⁺: 426.1.

### Step 5: N-(2,2-difluorobenzo[d][1,3]dioxol-5-yl)-3-(3-fluoro-5-(1-((2-(trimethylsilyl)ethoxy)methyl)-1H-[1,2,3]triazolo[4,5-b]pyridin-5-yl)pyridin-2-yl)morpholine-4-carboxamide

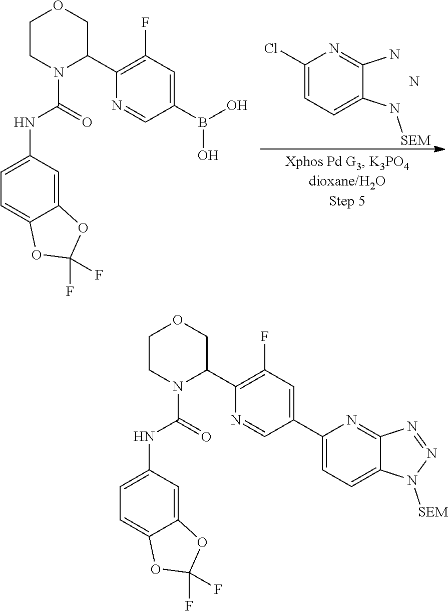

A solution of (6-(4-((2,2-difluorobenzo[d][1,3]dioxol-5-yl)carbamoyl)morpholin-3-yl)-5-fluoropyridin-3-yl)boronic acid (45.0 mg, 0.106 mmol), 5-chloro-1-((2-(trimethylsilyl)ethoxy)methyl)-1H-[1,2,3]triazolo[4,5-b]pyridine (30.0 mg, 0.106 mmol), Xphos Pd G3 (8.0 mg, 11 μmol), and K₃PO₄ (67.0 mg, 0.318 mmol) in 1,4-dioxane (5.0 mL) and water (1.0 mL) was stirred at 80° C. for 2 hours under an atmosphere of nitrogen. Upon completion, the mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0%-100% over 30 min; detector: UV 254 nm) to afford N-(2,2-difluorobenzo[d][1,3]dioxol-5-yl)-3-(3-fluoro-5-(1-((2-(trimethylsilyl)ethoxy) methyl)-1H-[1,2,3]triazolo[4,5-b]pyridin-5-yl)pyridin-2-yl)morpholine-4-carboxamide (49.0 mg, 73%) as a white solid. LCMS (ESI) [M+H]⁺: 630.3.

### Step 6: 3-(5-(1H-[1,2,3]triazolo[4,5-b]pyridin-5-yl)-3-fluoropyridin-2-yl)-N-(2,2-difluorobenzo[d][1,3]dioxol-5-yl)morpholine-4-carboxamide

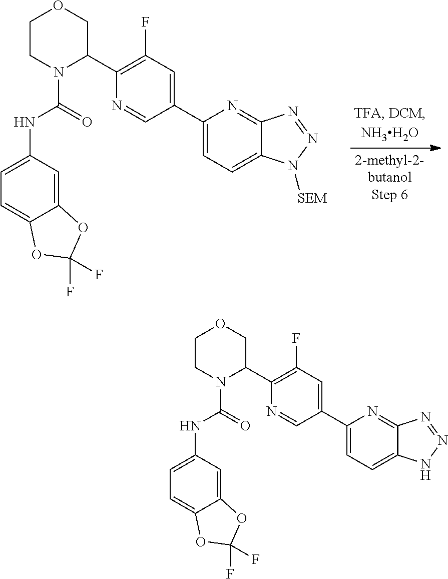 [SMILES: C[Si](C)(C)CCOCn1nnc2nc(-c3cnc(C4COCCN4C(=O)Nc4ccc5c(c4)OC(F)(F)O5)c(F)c3)ccc21 ; O=C(Nc1ccc2c(c1)OC(F)(F)O2)N1CCOCC1c1ncc(-c2ccc3[nH]nnc3n2)cc1F]

A solution of N-(2,2-difluorobenzo[d][1,3]dioxol-5-yl)-3-(3-fluoro-5-(1-((2-(trimethylsilyl)ethoxy)methyl)-1H-[1,2,3]triazolo[4,5-b]pyridin-5-yl)pyridin-2-yl)morpholine-4-carboxamide (44.0 mg, 0.070 mmol) in TFA (2.0 mL) and dichloromethane (2.0 mL) was stirred at room temperature. After 2 hours, the resultant mixture was concentrated under reduced pressure. Then, 2-methyl-2-butanol (3.0 mL) and concentrated ammonium hydroxide (1.0 mL) were added. The resultant mixture was stirred at room temperature for an additional 2 hours. Upon completion, the mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0%-100% over 30 min; detector: UV 254 nm) to afford 3-(5-(1H-[1,2,3]triazolo[4,5-b]pyridin-5-yl)-3-fluoropyridin-2-yl)-N-(2,2-difluorobenzo[d][1,3]dioxol-5-yl)morpholine-4-carboxamide (26 mg, 74%) as a pale-yellow solid. LCMS (ESI) [M+H]⁺: 500.1.

### Step 6: (R)-3-(5-(1H-[1,2,3]triazolo[4,5-b]pyridin-5-yl)-3-fluoropyridin-2-yl)-N-(2,2-difluorobenzo[d][1,3]dioxol-5-yl)morpholine-4-carboxamide and (S)-3-(5-(1H-[1,2,3]triazolo[4,5-b]pyridin-5-yl)-3-fluoropyridin-2-yl)-N-(2,2-difluorobenzo[d][1,3]dioxol-5-yl)morpholine-4-carboxamide

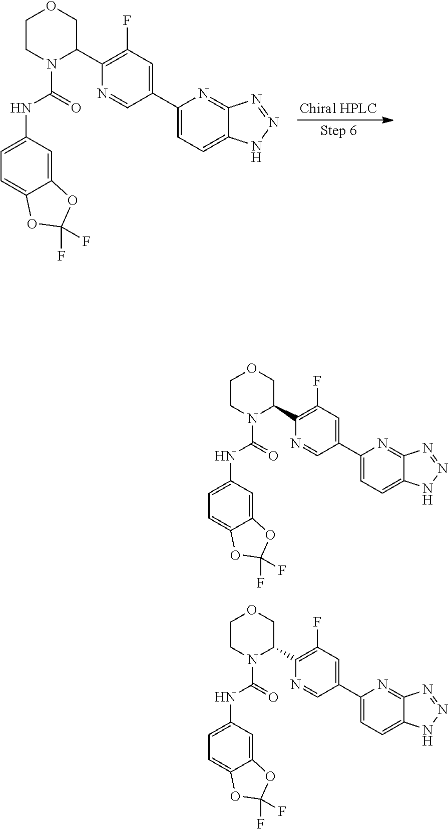

3-(5-(1H-[1,2,3]triazolo[4,5-b]pyridin-5-yl)-3-fluoropyridin-2-yl)-N-(2,2-difluorobenzo[d][1,3]dioxol-5-yl)morpholine-4-carboxamide (26 mg) was resolved by preparative chiral HPLC (conditions: column: CHIRAL ART Cellulose-SZ, 3*25 cm, 5 μm; mobile phase A: hexane (0.1% FA), mobile phase B: ethanol; flow rate: 40 mL/min; gradient (B %): isocratic, 45% B; detector: 220/254 nm; RT1 (min): 6.5; RT2 (min): 8) to afford (Compound 316a; Method BE, Peak 1, 0.71 min) (R)-3-(5-(1H-[1,2,3]triazolo[4,5-b]pyridin-5-yl)-3-fluoropyridin-2-yl)-N-(2,2-difluorobenzo[d][1,3]dioxol-5-yl)morpholine-4-carboxamide (1^{st} peak, 7.3 mg, 29%) as a white solid and (Compound 316b; Method BE, Peak 1, 0.91 min) (S)-3-(5-(1H-[1,2,3]triazolo[4,5-b]pyridin-5-yl)-3-fluoropyridin-2-yl)-N-(2,2-difluorobenzo[d][1,3]dioxol-5-yl)morpholine-4-carboxamide (2^{nd} peak, 7.2 mg, 28%) as a white solid.

| Compound# | Characterization Data |
|---|---|
| 316a | LCMS (ESI) [M + H]⁺: 500.1 |
| | ¹H NMR (400 MHz, DMSO-d₆) δ 9.17 (s, 1H), 8.84 (s, 1H), 8.58 (d, J = 8.8 Hz, 1H), 8.45- |
| | 8.41 (m, 1H), 8.20 (d, J = 8.8 Hz, 1H), 7.56 (s, 1H), 7.25 (d, J = 8.8 Hz, 1H), 7.18-7.15 (m, |
| | 1H), 5.48 (s, 1H), 4.29 (d, J = 11.6 Hz, 1H), 3.98-3.92 (m, 4H), 3.65-3.55 (m, 1H). |
| 316b | LCMS (ESI) [M + H]⁺: 500.1 |
| | ¹H NMR (400 MHz, DMSO-d₆) δ 9.17 (s, 1H), 8.84 (s, 1H), 8.58 (d, J = 8.8 Hz, 1H), 8.45- |
| | 8.42 (m, 1H), 8.21 (d, J = 8.8 Hz, 1H), 7.56 (s, 1H), 7.25 (d, J = 8.8 Hz, 1H), 7.17 (d, J = 8.8 |
| | Hz, 1H), 5.48 (s, 1H), 4.29 (d, J = 12.0 Hz, 1H), 3.97-3.92 (m, 4H), 3.63-3.61 (m, 1H). |

### Example 317: (S)-2-(5-(1H-[1,2,3]triazolo[4,5-b]pyridin-5-yl)-3-fluoropyridin-2-yl)-N-(5-fluoro-6-(1-(trifluoromethyl)cyclopropyl)pyridin-3-yl)pyrrolidine-1-carboxamide and (R)-2-(5-(1H-[1,2,3]triazolo[4,5-b]pyridin-5-yl)-3-fluoropyridin-2-yl)-N-(5-fluoro-6-(1-(trifluoromethyl)cyclopropyl)pyridin-3-yl)pyrrolidine-1-carboxamide

### Step 1: (1-(tert-butoxycarbonyl)pyrrolidin-2-yl)-5-fluoropyridin-3-yl)boronic acid

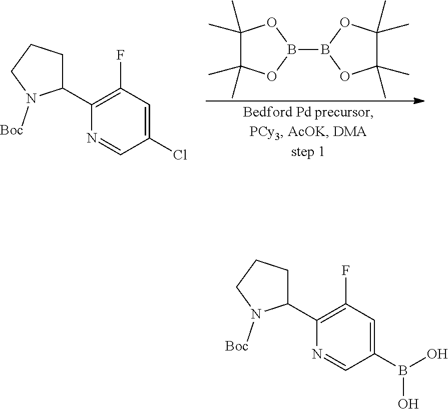

A solution of tert-butyl 2-(5-chloro-3-fluoropyridin-2-yl)pyrrolidine-1-carboxylate (2 g, 6.6 mmol), potassium acetate (1.9 g, 19.9 mmol), PCy₃ (0.5 g, 1.99 mmol), Bedford Pd precursor (0.10 g, 0.06 mmol) in DMA (10 mL) was stirred at 90° C. for 12 hours under an atmosphere of nitrogen. Upon completion, the resulting mixture was concentrated under reduced pressure. The crude residue was directly purified by reversed-phase flash (conditions: column: C18 silica gel; mobile phase A: water (0.1% formic acid); mobile phase B: MeCN; gradient (B %): 0% to 100% over 30 min; detector: UV 254 nm) to afford (1-(tert-butoxycarbonyl)pyrrolidin-2-yl)-5-fluoropyridin-3-yl)boronic acid (1.2 g, 58%) as a white solid. LCMS (ESI) [M+H]⁺: 311.1.

### Step 2: (5-fluoro-6-(pyrrolidin-2-yl)pyridin-3-yl)boronic acid

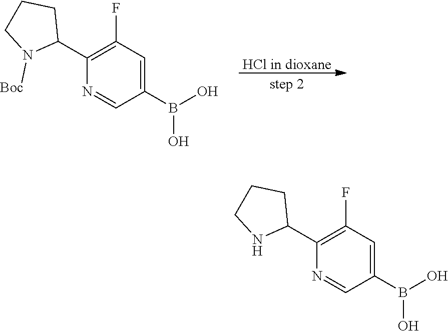 [SMILES: CC(C)(C)OC(=O)N1CCCC1c1ncc(B(O)O)cc1F ; "HCl in dioxane" ; OB(O)c1cnc(C2CCCN2)c(F)c1]

HCl (13 mL, 52 mmol, 4 M in 1,4-dioxane) was added into a solution of (6-(1-(tert-butoxycarbonyl)pyrrolidin-2-yl)-5-fluoropyridin-3-yl)boronic acid (0.800 g, 2.58 mmol) in 1,4-dioxane (10 mL) at room temperature. After 2 hours, the resulting mixture was concentrated under reduced pressure to afford crude (5-fluoro-6-(pyrrolidin-2-yl)pyridin-3-yl)boronic acid (500 mg, 92%) as a white solid, which was used directly in the next step without further purification. LCMS (ESI) [M+H]⁺: 210.9

### Step 3: 5-(5-fluoro-6-(pyrrolidin-2-yl)pyridin-3-yl)-1-((2-(trimethylsilyl)ethoxy)methyl)-1H-[1,2,3]triazolo[4,5-b]pyridine

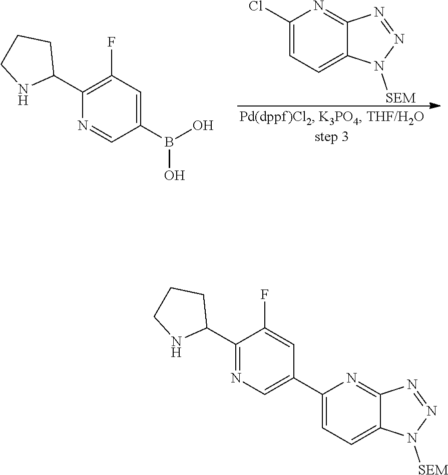 [SMILES: OB(O)c1cnc(C2CCCN2)c(F)c1 ; C[Si](C)(C)CCOCn1nnc2nc(Cl)ccc21 ; C[Si](C)(C)CCOCn1nnc2nc(-c3cnc(C4CCCN4)c(F)c3)ccc21]

A solution of (5-fluoro-6-(pyrrolidin-2-yl)pyridin-3-yl)boronic acid (500 mg, 2.38 mmol), 5-chloro-1-((2-(trimethylsilyl)ethoxy)methyl)-1H-[1,2,3]triazolo[4,5-b]pyridine (746 mg, 2.61 mmol), Pd(dppf)Cl₂ (348 mg, 0.47 mmol), K₃PO₄ (1.5 g, 7.14 mmol) in THF (10 mL) and H₂O (1 mL) was stirred at 85° C. for 5 hours under an atmosphere of nitrogen. Upon completion, the resulting mixture was concentrated under reduced pressure. The crude residue was directly purified by silica gel column chromatography, eluted with dichloromethane/methanol (5:1) to afford 5-(5-fluoro-6-(pyrrolidin-2-yl)pyridin-3-yl)-1-((2-(trimethylsilyl)ethoxy)methyl)-1H-[1,2,3]triazolo[4,5-b]pyridine (600 mg, 60%) as a black oil. LCMS (ESI) [M+H]⁺: 415.1.

### Step 4: 2-(3-fluoro-5-(1-((2-(trimethylsilyl)ethoxy)methyl)-1H-[1,2,3]triazolo[4,5-b]pyridin-5-yl)pyridin-2-yl)-N-(5-fluoro-6-(1-(trifluoromethyl)cyclopropyl)pyridin-3-yl)pyrrolidine-1-carboxamide

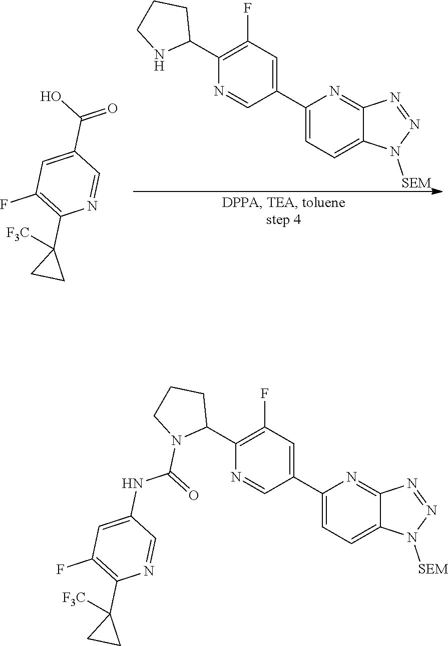 [SMILES: O=C(O)c1cnc(C2(C(F)(F)F)CC2)c(F)c1 ; C[Si](C)(C)CCOCn1nnc2nc(-c3cnc(C4CCCN4)c(F)c3)ccc21 ; C[Si](C)(C)CCOCn1nnc2nc(-c3cnc(C4CCCN4C(=O)Nc4cnc(C5(C(F)(F)F)CC5)c(F)c4)c(F)c3)ccc21]

A solution of 5-fluoro-6-[1-(trifluoromethyl)cyclopropyl]pyridine-3-carboxylic acid (59.5 mg, 0.23 mmol), DPPA (119 mg, 0.43 mmol), triethylamine (65.9 mg, 0.65 mmol) and 5-(5-fluoro-6-(pyrrolidin-2-yl)pyridin-3-yl)-1-((2-(trimethylsilyl)ethoxy)methyl)-1H-[1,2,3]triazolo[4,5-b]pyridine (90.0 mg, 0.21 mmol) in toluene (3.0 mL) was stirred at 80° C. for 1 hour. Upon completion, the resulting mixture was concentrated under reduced pressure. The crude residue was directly purified by silica gel column chromatography, eluted with dichloromethane/methanol (10:1) to afford 2-(3-fluoro-5-(1-((2-(trimethylsilyl)ethoxy)methyl)-1H-[1,2,3]triazolo[4,5-b]pyridin-5-yl)pyridin-2-yl)-N-(5-fluoro-6-(1-(trifluoromethyl)cyclopropyl)pyridin-3-yl)pyrrolidine-1-carboxamide (80 mg, 50%) as a yellow oil. LCMS (ESI) [M+H]⁺: 661.2.

### Step 5: 2-(5-(1H-[1,2,3]triazolo[4,5-b]pyridin-5-yl)-3-fluoropyridin-2-yl)-N-(5-fluoro-6-(1-(trifluoromethyl)cyclopropyl)pyridin-3-yl)pyrrolidine-1-carboxamide

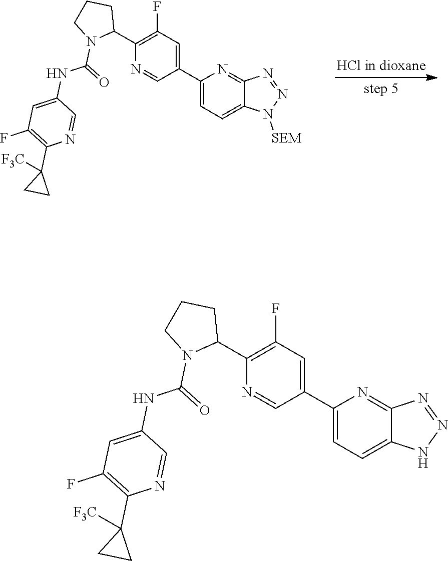 [SMILES: C[Si](C)(C)CCOCn1nnc2nc(-c3cnc(C4CCCN4C(=O)Nc4cnc(C5(C(F)(F)F)CC5)c(F)c4)c(F)c3)ccc21 ; O=C(Nc1cnc(C2(C(F)(F)F)CC2)c(F)c1)N1CCCC1c1ncc(-c2ccc3[nH]nnc3n2)cc1F]

HCl (2.2 mL, 8.8 mmol, 4 M in 1,4-dioxane) was added into a solution of 2-(3-fluoro-5-(1-((2-(trimethylsilyl)ethoxy)methyl)-1H-[1,2,3]triazolo[4,5-b]pyridin-5-yl)pyridin-2-yl)-N-(5-fluoro-6-(1-(trifluoromethyl)cyclopropyl)pyridin-3-yl)pyrrolidine-1-carboxamide (80.0 mg, 0.121 mmol) in 1,4-dioxane (2 mL) at room temperature. After 2 hours, the resulting mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 25% to 100% over 25 min; detector: UV 254 nm) to afford 2-(5-(1H-[1,2,3]triazolo[4,5-b]pyridin-5-yl)-3-fluoropyridin-2-yl)-N-(5-fluoro-6-(1-(trifluoromethyl)cyclopropyl)pyridin-3-yl)pyrrolidine-1-carboxamide (46 mg, 72%) as a white solid. LCMS (ESI) [M+H]⁺: 531.1.

### Step 6: (S)-2-(5-(1H-[1,2,3]triazolo[4,5-b]pyridin-5-yl)-3-fluoropyridin-2-yl)-N-(5-fluoro-6-(1-(trifluoromethyl)cyclopropyl)pyridin-3-yl)pyrrolidine-1-carboxamide and (R)-2-(5-(1H-[1,2,3]triazolo[4,5-b]pyridin-5-yl)-3-fluoropyridin-2-yl)-N-(5-fluoro-6-(1-(trifluoromethyl)cyclopropyl)pyridin-3-yl)pyrrolidine-1-carboxamide

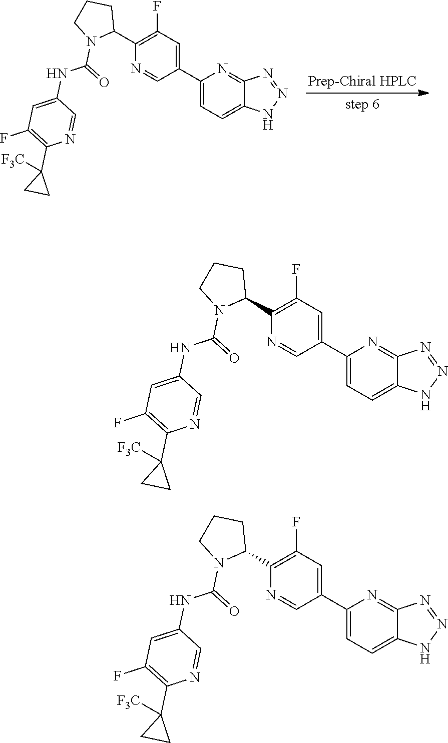 [SMILES: O=C(Nc1cnc(C2(C(F)(F)F)CC2)c(F)c1)N1CCCC1c1ncc(-c2ccc3[nH]nnc3n2)cc1F ; O=C(Nc1cnc(C2(C(F)(F)F)CC2)c(F)c1)N1CCC[C@H]1c1ncc(-c2ccc3[nH]nnc3n2)cc1F ; O=C(Nc1cnc(C2(C(F)(F)F)CC2)c(F)c1)N1CCC[C@@H]1c1ncc(-c2ccc3[nH]nnc3n2)cc1F]

2-(5-(1H-[1,2,3]triazolo[4,5-b]pyridin-5-yl)-3-fluoropyridin-2-yl)-N-(5-fluoro-6-(1-(trifluoromethyl)cyclopropyl)pyridin-3-yl)pyrrolidine-1-carboxamide (46 mg) was resolved by preparative chiral HPLC (conditions: Column: Lux 5 μm Cellulose-2 3*25 cm, 5 μm; Mobile Phase A: Hexane (0.1% formic acid), Mobile Phase B: EtOH; Flow rate: 40 mL/min; Gradient (B %): isocratic, 30% B; Wave Length: 220/254 nm; RT1 (min): 12.6; RT2(min): 14.8) to afford (Compound 317a) (S)-2-(5-(1H-[1,2,3]triazolo[4,5-b]pyridin-5-yl)-3-fluoropyridin-2-yl)-N-(5-fluoro-6-(1-(trifluoromethyl)cyclopropyl)pyridin-3-yl)pyrrolidine-1-carboxamide (1^{st} peak, 9.8 mg, 21%; Method BX, Peak 1, 2.20 min) as a white solid and (Compound 317b) (R)-2-(5-(1H-[1,2,3]triazolo[4,5-b]pyridin-5-yl)-3-fluoropyridin-2-yl)-N-(5-fluoro-6-(1-(trifluoromethyl)cyclopropyl)pyridin-3-yl)pyrrolidine-1-carboxamide (2^{nd} peak, 19.7 mg, 43%; Method BX, Peak 2, 2.66 min) as a white solid.

| Cmpd # | Characterization Data |
|---|---|
| 317a | LCMS (ESI) [M + H]⁺: 531.1 |
| | ¹H NMR (400 MHz, DMSO-d₆) δ 9.12 (s, 1H), 8.89 (s, 1H), 8.55 (d, J = 8.7 Hz, 1H), 8.49 (s, 1H), |
| | 8.37 (dd, J = 11.5, 1.8 Hz, 1H), 8.15 (d, J = 8.7 Hz, 1H), 7.90 (dd, J = 12.8, 2.1 Hz, 1H), 5.39 (d, J = |
| | 7.0 Hz, 1H), 3.84-3.77 (m, 1H), 3.75-3.62 (m, 1H), 2.43-2.37 (m, 1H), 2.23-1.92 (m, |
| | 3H), 1.36 (d, J = 2.3 Hz, 2H), 1.23 (s, 2H). |
| 317b | LCMS (ESI) [M + H]⁺: 531.1 |
| | ¹H NMR (400 MHz, DMSO-d₆) δ 9.12 (d, J = 1.5 Hz, 1H), 8.89 (s, 1H), 8.57 (d, J = 8.7 Hz, 1H), |
| | 8.49 (s, 1H), 8.38 (dd, J = 11.5, 1.8 Hz, 1H), 8.18 (d, J = 8.7 Hz, 1H), 7.90 (dd, J = 12.8, 2.0 Hz, |
| | 1H), 5.38 (t, J = 5.9 Hz, 1H), 3.86-3.74 (m, 1H), 3.68 (q, J = 7.7 Hz, 1H), 2.45-2.34 (m, 1H), |
| | 2.18-1.88 (m, 3H), 1.36 (d, J = 2.2 Hz, 2H), 1.15 (s, 2H). |

### Example 318: (S)-2-(5-(1H-[1,2,3]triazolo[4,5-b]pyridin-5-yl)-3-fluoropyridin-2-yl)-N-(4-(1-(trifluoromethyl)cyclopropyl)phenyl)pyrrolidine-1-carboxamide and (R)-2-(5-(1H-[1,2,3]triazolo[4,5-b]pyridin-5-yl)-3-fluoropyridin-2-yl)-N-(4-(1-(trifluoromethyl)cyclopropyl)phenyl)pyrrolidine-1-carboxamide

### Step 1: 2-(3-fluoro-5-(1-((2-(trimethylsilyl)ethoxy)methyl)-1H-[1,2,3]triazolo[4,5-b]pyridin-5-yl)pyridin-2-yl)-N-(4-(1-(trifluoromethyl)cyclopropyl)phenyl)pyrrolidine-1-carboxamide

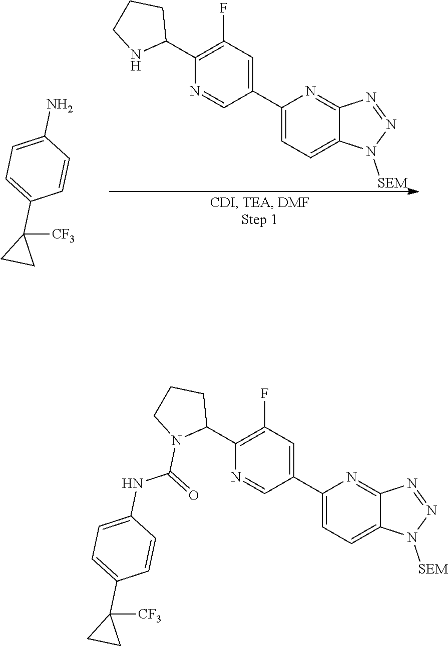 [SMILES: Nc1ccc(C2(C(F)(F)F)CC2)cc1 ; C[Si](C)(C)CCOCn1nnc2nc(-c3cnc(C4CCCN4)c(F)c3)ccc21 ; C[Si](C)(C)CCOCn1nnc2nc(-c3cnc(C4CCCN4C(=O)Nc4ccc(C5(C(F)(F)F)CC5)cc4)c(F)c3)ccc21]

A solution of 5-(5-fluoro-6-(pyrrolidin-2-yl)pyridin-3-yl)-1-((2-(trimethylsilyl)ethoxy)methyl)-1H-[1,2,3]triazolo[4,5-b]pyridine (100 mg, 0.241 mmol), triethylamine (73.2 mg, 0.723 mmol), CDI (78.2 mg, 0.482 mmol) and 4-(1-(trifluoromethyl)cyclopropyl)aniline (48.5 mg, 0.241 mmol) in DMF (2 mL) was stirred at 25° C. for 1 hour. Upon completion, the reaction mixture was concentrated under reduced pressure. The crude residue was directly purified by reverse phase flash chromatography (conditions: column: C18 silica gel; mobile phase A: water (0.1% formic acid); mobile phase B: MeCN; gradient (B %): 0% to 100% over 25 min; detector: UV 254 nm) to give 2-(3-fluoro-5-(1-((2-(trimethylsilyl)ethoxy)methyl)-1H-[1,2,3]triazolo[4,5-b]pyridin-5-yl)pyridin-2-yl)-N-(4-(1-(trifluoromethyl)cyclopropyl)phenyl)pyrrolidine-1-carboxamide (60 mg, 39%) as a yellow green solid. LCMS (ESI) [M+H]⁺: 642.2

### Step 2: 2-(5-(1H-[1,2,3]triazolo[4,5-b]pyridin-5-yl)-3-fluoropyridin-2-yl)-N-(4-(1-(trifluoromethyl)cyclopropyl)phenyl)pyrrolidine-1-carboxamide

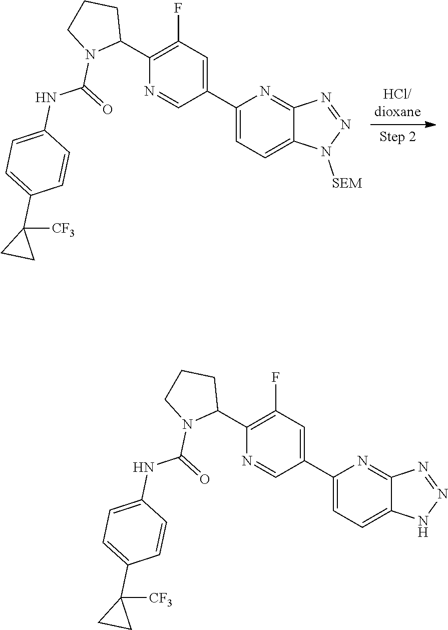 [SMILES: C[Si](C)(C)CCOCn1nnc2nc(-c3cnc(C4CCCN4C(=O)Nc4ccc(C5(C(F)(F)F)CC5)cc4)c(F)c3)ccc21 ; O=C(Nc1ccc(C2(C(F)(F)F)CC2)cc1)N1CCCC1c1ncc(-c2ccc3[nH]nnc3n2)cc1F]

HCl (2.2 mL, 8.8 mmol, 4 M in 1,4-dioxane) was added into a solution of 2-(3-fluoro-5-(1-((2-(trimethylsilyl)ethoxy)methyl)-1H-[1,2,3]triazolo[4,5-b]pyridin-5-yl)pyridin-2-yl)-N-(4-(1-(trifluoromethyl)cyclopropyl)phenyl)pyrrolidine-1-carboxamide (60.0 mg, 0.093 mmol) in 1,4-dioxane (2 mL) and the mixture was stirred at room temperature for 2 hours. Upon completion, the resulting mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 25% to 100% over 25 min; detector: UV 254 nm) to afford 2-(5-(1H-[1,2,3]triazolo[4,5-b]pyridin-5-yl)-3-fluoropyridin-2-yl)-N-(4-(1-(trifluoromethyl)cyclopropyl)phenyl)pyrrolidine-1-carboxamide (30 mg, 63%) as a white solid. LCMS (ESI) [M+H]⁺: 512.1.

### Step 3: (S)-2-(5-(1H-[1,2,3]triazolo[4,5-b]pyridin-5-yl)-3-fluoropyridin-2-yl)-N-(4-(1-(trifluoromethyl)cyclopropyl)phenyl)pyrrolidine-1-carboxamide and (R)-2-(5-(1H-[1,2,3]triazolo[4,5-b]pyridin-5-yl)-3-fluoropyridin-2-yl)-N-(4-(1-(trifluoromethyl)cyclopropyl)phenyl)pyrrolidine-1-carboxamide

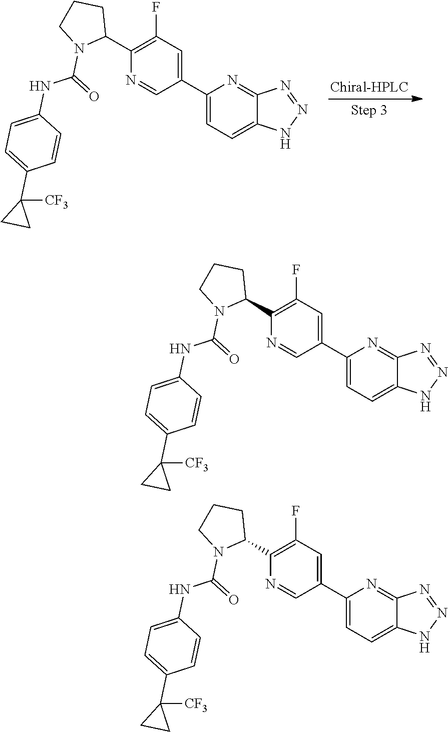 [SMILES: O=C(Nc1ccc(C2(C(F)(F)F)CC2)cc1)N1CCCC1c1ncc(-c2ccc3[nH]nnc3n2)cc1F ; O=C(Nc1ccc(C2(C(F)(F)F)CC2)cc1)N1CCC[C@H]1c1ncc(-c2ccc3[nH]nnc3n2)cc1F ; O=C(Nc1ccc(C2(C(F)(F)F)CC2)cc1)N1CCC[C@@H]1c1ncc(-c2ccc3[nH]nnc3n2)cc1F]

2-(5-(1H-[1,2,3]triazolo[4,5-b]pyridin-5-yl)-3-fluoropyridin-2-yl)-N-(4-(1-(trifluoromethyl)cyclopropyl)phenyl)pyrrolidine-1-carboxamide (30 mg) was resolved by preparative chiral HPLC (conditions: Column: CHIRAL ART Cellulose-SZ, 3*25 cm, 5 μm; Mobile Phase A: Hexane (0.1% FA), Mobile Phase B: EtOH; Flow rate: 40 mL/min; Gradient (B %): isocratic, 50% B; Wave Length: 220/254 nm; RT1 (min): 6.09; RT2 (min): 7.30) to afford (Compound 318a) (S)-2-(5-(1H-[1,2,3]triazolo[4,5-b]pyridin-5-yl)-3-fluoropyridin-2-yl)-N-(4-(1-(trifluoromethyl)cyclopropyl)phenyl)pyrrolidine-1-carboxamide (1^{st} peak, 6.9 mg, 23%; Method BE, Peak 1, 0.77 min) as a white solid and (Compound 318b) (R)-2-(5-(1H-[1,2,3]triazolo[4,5-b]pyridin-5-yl)-3-fluoropyridin-2-yl)-N-(4-(1-(trifluoromethyl)cyclopropyl)phenyl)pyrrolidine-1-carboxamide (2^{nd} peak, 18.1 mg, 60%; Method BE, Peak 2, 0.97 min) as a white solid.

| Cmpd # | Characterization Data |
|---|---|
| 318a | LCMS (ESI) [M + H]⁺: 512.2 |
| | ¹H NMR (400 MHz, DMSO-d₆) δ 9.12 (s, 1H), 8.54 (d, J = 7.3 Hz, 1H), 8.35 (d, J = |
| | 11.5 Hz, 2H), 8.14 (d, J = 9.3 Hz, 1H), 7.51-7.41 (m, 2H), 7.31-7.19 (m, 2H), 5.37 |
| | (s, 1H), 3.84-3.77 (m, 1H), 3.70-3.58 (m, 1H), 2.41-2.32 (m, 1H), 2.16-1.88 (m, |
| | 3H), 1.25 (s, 2H), 1.02 (s, 2H). |
| 318b | LCMS (ESI) [M + H]⁺: 512.1 |
| | ¹H NMR (400 MHz, DMSO-d₆) δ 9.12 (s, 1H), 8.56 (d, J = 8.6 Hz, 1H), 8.36 (d, J = |
| | 10.6 Hz, 2H), 8.16 (d, J = 8.7 Hz, 1H), 7.53-7.37 (m, 2H), 7.26 (d, J = 8.2 Hz, 2H), |
| | 5.41-5.32 (m, 1H), 3.82-3.74 (m, 1H), 3.65 (q, J = 8.6, 8.1 Hz, 1H), 2.42-2.31 (m, |
| | 1H), 2.20-1.86 (m, 3H), 1.31-1.20 (m, 2H), 1.02 (s, 2H). |

### Example 319: (R)-2-(5-(1H-[1,2,3]triazolo[4,5-b]pyridin-5-yl)-3-fluoropyridin-2-yl)-N-(4-cyclopropyl-3-fluorophenyl)pyrrolidine-1-carboxamide

### Step 1: 4-cyclopropyl-3-fluoroaniline

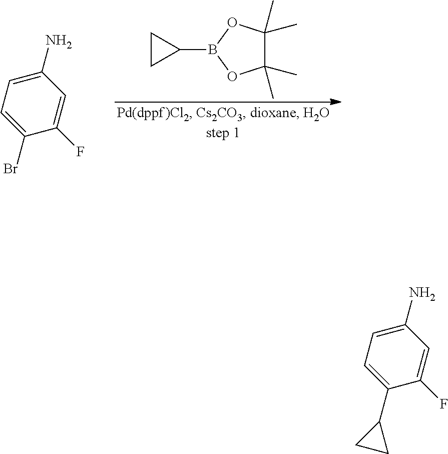

A solution of 4-bromo-3-fluoroaniline (3.0 g, 15.8 mmol), 2-cyclopropyl-4,4,5,5-tetramethyl-1,3,2-dioxaborolane (5.31 g, 31.6 mmol), Cs₂CO₃ (15.4 g, 47.4 mmol) and Pd(dppf)Cl₂ (1.16 g, 1.58 mmol) in 1,4-dioxane (30 mL) and H₂O (6 mL) was stirred at 100° C. for 16 hours under an atmosphere of nitrogen. Upon completion, the resulting mixture was diluted with water (60 mL) and extracted with ethyl acetate (3×80 mL). The combined organic layers were concentrated under reduced pressure. The crude residue was purified by silica gel column chromatography, eluted with petroleum ether/ethyl acetate (2:1) to afford 4-cyclopropyl-3-fluoroaniline (2.0 g, 84%) as a light-yellow oil. LCMS (ESI) [M+H]⁺: 152.1.

### Step 2: (R)-2-(5-chloro-3-fluoropyridin-2-yl)-N-(4-cyclopropyl-3-fluorophenyl)pyrrolidine-1-carboxamide

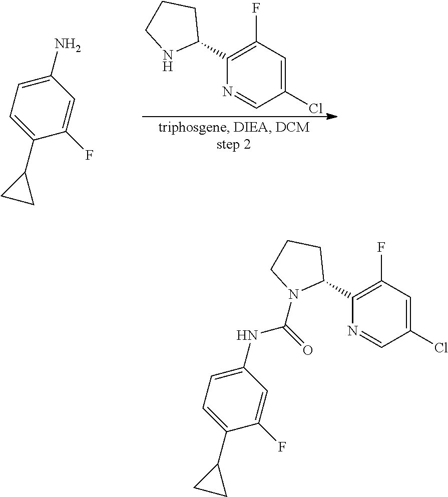 [SMILES: Nc1ccc(C2CC2)c(F)c1 ; Fc1cc(Cl)cnc1[C@H]1CCCN1 ; O=C(Nc1ccc(C2CC2)c(F)c1)N1CCC[C@@H]1c1ncc(Cl)cc1F]

To a stirred solution of 4-cyclopropyl-3-fluoroaniline (1 g, 6.61 mmol) and DIEA (2.56 g, 19.8 mmol) in DCM (20 mL) was added (R)-5-chloro-3-fluoro-2-(pyrrolidin-2-yl)pyridine (1.99 g, 9.92 mmol) and triphosgene (785 mg, 2.65 mmol) at 0° C. The resulting mixture was warmed to room temperature and stirred for 1 hour. Upon completion, the resulting mixture was poured into water (60 mL) and extracted with DCM (3×80 mL). The combined organic layers were concentrated under reduced pressure. The crude residue was purified by silica gel column chromatography, eluted with petroleum ether/ethyl acetate (3:1) to afford (R)-2-(5-chloro-3-fluoropyridin-2-yl)-N-(4-cyclopropyl-3-fluorophenyl)pyrrolidine-1-carboxamide (1.0 g, 40%) as a white solid. LCMS (ESI) [M+H]⁺: 378.1

### Step 3: (R)-(6-(1-((4-cyclopropyl-3-fluorophenyl)carbamoyl)pyrrolidin-2-yl)-5-fluoropyridin-3-yl)boronic acid

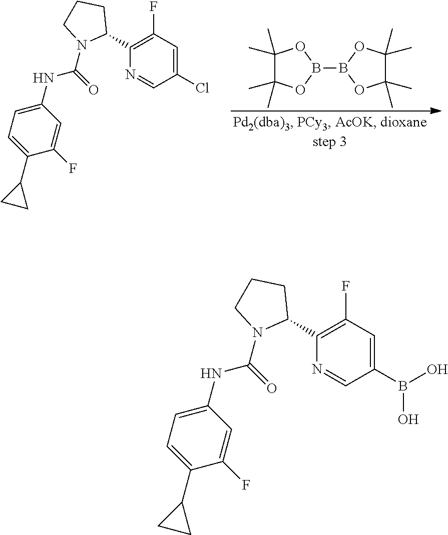

A stirred solution of (R)-2-(5-chloro-3-fluoropyridin-2-yl)-N-(4-cyclopropyl-3-fluorophenyl)pyrrolidine-1-carboxamide (1.0 g, 2.65 mmol), 4,4,4′,4′,5,5,5′,5′-octamethyl-2,2′-bi(1,3,2-dioxaborolane) (806 mg, 3.18 mmol), Pd₂(dba)₃ (242 mg, 0.265 mmol), tricyclohexylphosphane (297 mg, 1.06 mmol) and potassium acetate (779 mg, 7.94 mmol) in 1,4-dioxane (20 mL) was stirred at 110° C. for 6 hours. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 40 min; detector: UV 254 nm) to afford (R)-(6-(1-((4-cyclopropyl-3-fluorophenyl)carbamoyl)pyrrolidin-2-yl)-5-fluoropyridin-3-yl)boronic acid (500 mg, 40%) as a light yellow solid. LCMS (ESI) [M+H]⁺: 388.2.

### Step 4: (R)—N-(4-cyclopropyl-3-fluorophenyl)-2-(3-fluoro-5-(1-((2-(trimethylsilyl)ethoxy)methyl)-1H-[1,2,3]triazolo[4,5-b]pyridin-5-yl)pyridin-2-yl)pyrrolidine-1-carboxamide

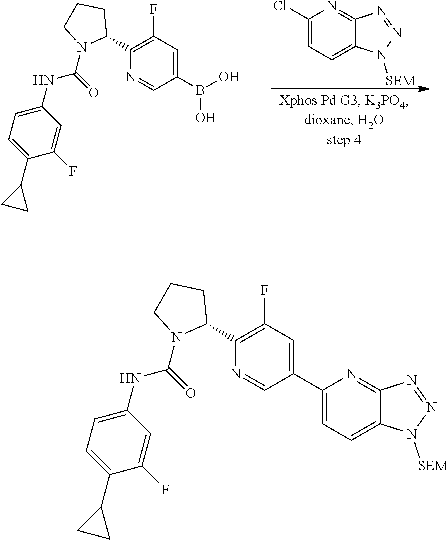 [SMILES: O=C(Nc1ccc(C2CC2)c(F)c1)N1CCC[C@@H]1c1ncc(B(O)O)cc1F ; C[Si](C)(C)CCOCn1nnc2nc(Cl)ccc21 ; C[Si](C)(C)CCOCn1nnc2nc(-c3cnc([C@H]4CCCN4C(=O)Nc4ccc(C5CC5)c(F)c4)c(F)c3)ccc21]

A solution of (R)-(6-(1-((4-cyclopropyl-3-fluorophenyl)carbamoyl)pyrrolidin-2-yl)-5-fluoropyridin-3-yl)boronic acid (100 mg, 0.258 mmol), 5-chloro-1-((2-(trimethylsilyl)ethoxy)methyl)-1H-[1,2,3]triazolo[4,5-b]pyridine (110 mg, 0.387 mmol), XPhos Pd G3 (21.9 mg, 0.026 mmol) and K₃PO₄ (164 mg, 0.774 mmol) in 1,4-dioxane (3 mL) and H₂O (0.6 mL) was stirred at 110° C. for 4 hours under an atmosphere of nitrogen. Upon completion, the reaction mixture was concentrated under reduced pressure. The crude residue was directly purified by silica gel column chromatography, eluted with petroleum ether/ethyl acetate (1:4) to afford (R)—N-(4-cyclopropyl-3-fluorophenyl)-2-(3-fluoro-5-(1-((2-(trimethylsilyl)ethoxy)methyl)-1H-[1,2,3]triazolo[4,5-b]pyridin-5-yl)pyridin-2-yl)pyrrolidine-1-carboxamide (120 mg, 78%) as a light yellow solid. LCMS (ESI) [M+H]⁺: 592.3.

### Step 5: (R)-2-(5-(1H-[1,2,3]triazolo[4,5-b]pyridin-5-yl)-3-fluoropyridin-2-yl)-N-(4-cyclopropyl-3-fluorophenyl)pyrrolidine-1-carboxamide

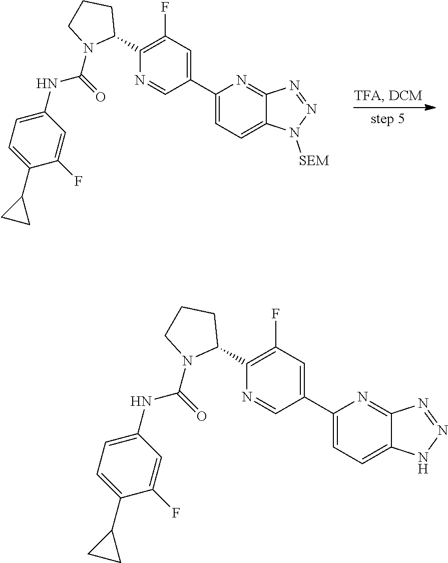 [SMILES: C[Si](C)(C)CCOCn1nnc2nc(-c3cnc([C@H]4CCCN4C(=O)Nc4ccc(C5CC5)c(F)c4)c(F)c3)ccc21 ; O=C(Nc1ccc(C2CC2)c(F)c1)N1CCC[C@@H]1c1ncc(-c2ccc3[nH]nnc3n2)cc1F]

To a stirred solution of (R)—N-(4-cyclopropyl-3-fluorophenyl)-2-(3-fluoro-5-(1-((2-(trimethylsilyl)ethoxy)methyl)-1H-[1,2,3]triazolo[4,5-b]pyridin-5-yl)pyridin-2-yl)pyrrolidine-1-carboxamide (100 mg, 0.169 mmol) in DCM (5 mL) was added TFA (1 mL) at 25° C. The resulting mixture was stirred at 25° C. for 2 hours. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 40 min; detector: UV 254 nm) to afford (Compound 319) (R)-2-(5-(1H-[1,2,3]triazolo[4,5-b]pyridin-5-yl)-3-fluoropyridin-2-yl)-N-(4-cyclopropyl-3-fluorophenyl)pyrrolidine-1-carboxamide (1^{st} peak, 52 mg, 66%) as a white solid.

| Cmpd # | Characterization Data |
|---|---|
| 319 | LCMS (ESI) [M + H]⁺: 462.2 |
| | ¹H NMR (400 MHz, DMSO-d₆) δ 9.13 (s, 1H), 8.58 (d, J = 8.8 Hz, 1H), 8.43-8.32 |
| | (m, 2H), 8.19 (d, J = 8.8 Hz, 1H), 7.38-7.34 (m, 1H), 7.15-7.12 (m, 1H), 6.80 (t, J = |
| | 8.8 Hz, 1H), 5.38-5.35 (m, 1H), 3.82-3.72 (m, 1H), 3.67-3.61 (m, 1H), 2.44-2.31 |
| | (m, 1H), 2.15-2.09 (m, 1H), 2.06-2.00 (m, 1H), 1.94-1.87 (m, 2H), 0.93-0.82 (m, |
| | 2H), 0.64-0.54 (m, 2H). |

### Example 320: (R)-2-(5-(1H-[1,2,3]triazolo[4,5-b]pyridin-5-yl)-3-fluoropyridin-2-yl)-N-(3-fluoro-4-(trifluoromethyl)phenyl)pyrrolidine-1-carboxamide

### Step 1: (R)-2-(5-chloro-3-fluoropyridin-2-yl)-N-(3-fluoro-4-(trifluoromethyl)phenyl)pyrrolidine-1-carboxamide

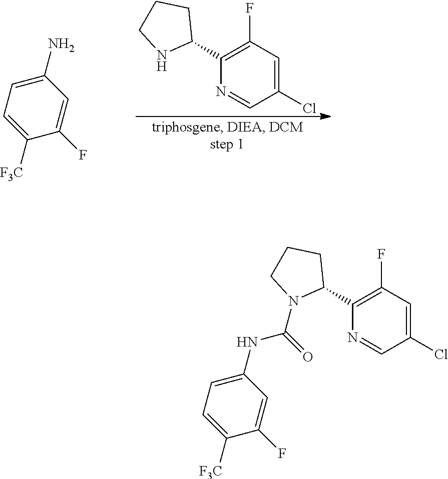 [SMILES: Nc1ccc(C(F)(F)F)c(F)c1 ; Fc1cc(Cl)cnc1[C@H]1CCCN1 ; O=C(Nc1ccc(C(F)(F)F)c(F)c1)N1CCC[C@@H]1c1ncc(Cl)cc1F]

A solution of 3-fluoro-4-(trifluoromethyl)aniline (2.0 g, 11.2 mmol), (R)-5-chloro-3-fluoro-2-(pyrrolidin-2-yl)pyridine (3.36 g, 16.7 mmol), triphosgene (1.33 g, 4.47 mmol) and N, N-diisopropylethylamine (4.33 g, 33.5 mmol) in dichloromethane (40.0 mL) was stirred at 25° C. for 3 hours. Upon completion, the mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 40 min; detector: UV 254 nm) to afford (R)-2-(5-chloro-3-fluoropyridin-2-yl)-N-(3-fluoro-4-(trifluoromethyl)phenyl)pyrrolidine-1-carboxamide (1.99 g, 44%) as a white solid. LCMS (ESI) [M+H]⁺: 406.1.

### Step 2: (R)-(5-fluoro-6-(1-((3-fluoro-4-(trifluoromethyl)phenyl)carbamoyl)pyrrolidin-2-yl)pyridin-3-yl)boronic acid

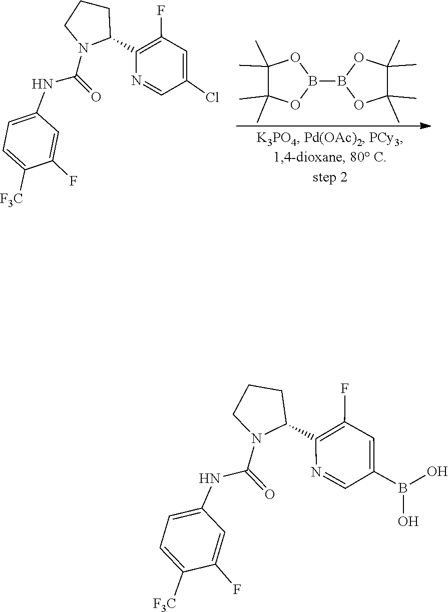

A solution of (R)-2-(5-chloro-3-fluoropyridin-2-yl)-N-(3-fluoro-4-(trifluoromethyl)phenyl)pyrrolidine-1-carboxamide (1.0 g, 2.47 mmol), 4,4,4′,4′,5,5,5′,5′-octamethyl-2,2′-bi(1,3,2-dioxaborolane) (938 mg, 3.70 mmol), Pd(II) acetate (55 mg, 0.246 mmol), tricyclohexylphosphine (207 mg, 0.739 mmol) and tribasic potassium phosphate (1.57 mg, 7.395 mmol) in 1,4-dioxane (15.0 mL) was stirred at 80° C. for 2 hours. Upon completion, the reaction mixture was concentrated under reduced pressure. The crude residue was directly purified by reversed phase column chromatography (conditions: column: C18 silica gel; mobile phase A: water (0.1% formic acid); mobile phase B: MeCN; gradient (B %): 0% to 100% over 40 min; detector: UV 254 nm) to afford (R)-(5-fluoro-6-(1-((3-fluoro-4-(trifluoromethyl)phenyl)carbamoyl)pyrrolidin-2-yl)pyridin-3-yl)boronic acid (680 mg, 66%) as a white solid. LCMS (ESI) [M+H]⁺: 416.0.

### Step 3: (R)—N-(3-fluoro-4-(trifluoromethyl)phenyl)-2-(3-fluoro-5-(1-((2-(trimethylsilyl)ethoxy)methyl)-1H-[1,2,3]triazolo[4,5-b]pyridin-5-yl)pyridin-2-yl)pyrrolidine-1-carboxamide

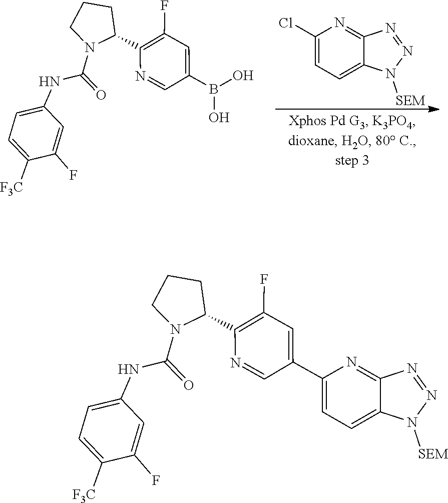 [SMILES: O=C(Nc1ccc(C(F)(F)F)c(F)c1)N1CCC[C@@H]1c1ncc(B(O)O)cc1F ; C[Si](C)(C)CCOCn1nnc2nc(Cl)ccc21 ; C[Si](C)(C)CCOCn1nnc2nc(-c3cnc([C@H]4CCCN4C(=O)Nc4ccc(C(F)(F)F)c(F)c4)c(F)c3)ccc21]

A solution of (R)-(5-fluoro-6-(1-((3-fluoro-4-(trifluoromethyl)phenyl)carbamoyl)pyrrolidin-2-yl)pyridin-3-yl)boronic acid (200 mg, 0.482 mmol), 5-chloro-1-((2-(trimethylsilyl)ethoxy)methyl)-1H-[1,2,3]triazolo[4,5-b]pyridine (165 mg, 0.578 mmol), tribasic potassium phosphate (307 mg, 1.45 mmol), and XPhos Pd G3 (40.8 mg, 0.048 mmol) in 1,4-dioxane (4.0 mL) and water (1.0 mL) was stirred at 80° C. for 3 hours. Upon completion, the reaction mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 40 min; detector: UV 254 nm) to afford (R)—N-(3-fluoro-4-(trifluoromethyl)phenyl)-2-(3-fluoro-5-(1-((2-(trimethylsilyl)ethoxy)methyl)-1H-[1,2,3]triazolo[4,5-b]pyridin-5-yl)pyridin-2-yl)pyrrolidine-1-carboxamide (210 mg, 70%) as a white solid. LCMS (ESI) [M+H]⁺: 620.1.

### Step 4: (R)-2-(5-(1H-[1,2,3]triazolo[4,5-b]pyridin-5-yl)-3-fluoropyridin-2-yl)-N-(3-fluoro-4-(trifluoromethyl)phenyl)pyrrolidine-1-carboxamide

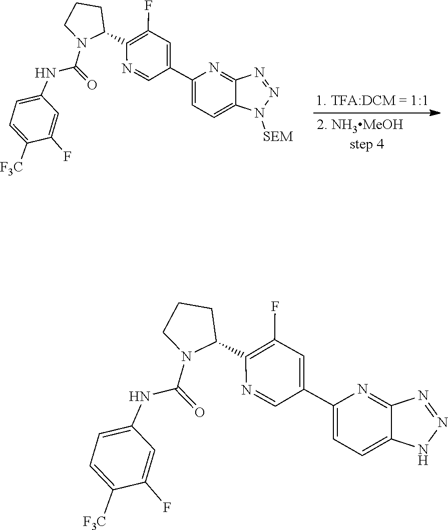 [SMILES: C[Si](C)(C)CCOCn1nnc2nc(-c3cnc([C@H]4CCCN4C(=O)Nc4ccc(C(F)(F)F)c(F)c4)c(F)c3)ccc21 ; O=C(Nc1ccc(C(F)(F)F)c(F)c1)N1CCC[C@@H]1c1ncc(-c2ccc3[nH]nnc3n2)cc1F]

To a solution of (R)—N-(3-fluoro-4-(trifluoromethyl)phenyl)-2-(3-fluoro-5-(1-((2-(trimethylsilyl)ethoxy)methyl)-1H-[1,2,3]triazolo[4,5-b]pyridin-5-yl)pyridin-2-yl)pyrrolidine-1-carboxamide (100 mg, 0.161 mmol) in dichloromethane (2.0 mL) were added TFA (2.0 mL) at room temperature under nitrogen atmosphere. After 2 hours, the resulting mixture was concentrated under reduced pressure. Then, NH₃ (2.0 mL, 4 M in MeOH) was added to the crude residue at room temperature. After stirring for 30 minutes, the resulting mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 40 min; detector: UV 254 nm) to afford (Compound 320) (R)-2-(5-(1H-[1,2,3]triazolo[4,5-b]pyridin-5-yl)-3-fluoropyridin-2-yl)-N-(3-fluoro-4-(trifluoromethyl)phenyl)pyrrolidine-1-carboxamide (1^{st} peak, 51.9 mg, 66%) as a white solid.

| Cmpd # | Characterization Data |
|---|---|
| 320 | LCMS (ESI) [M + H]⁺: 490.2 |
| | ¹H NMR (400 MHz, DMSO-d₆) δ 9.13 (s, 1H), 8.92 (s, 1H), 8.58 (d, J = 8.8 Hz, 1H), |
| | 8.40 (d, J = 1.6 Hz, 1H), 8.37 (d, J = 1.6 Hz, 1H), 7.69 (d, J = 14.4 Hz, 1H), 7.61- |
| | 7.49 (m, 1H), 7.45 (d, J = 8.8 Hz, 1H), 5.70-5.10 (m, 1H), 3.90-3.75 (m, 1H), 3.75- |
| | 3.59 (m, 1H), 2.46-2.35 (m, 1H), 2.21-2.00 (m, 2H), 2.00-1.87 (m, 1H). |

### Example 321: (S)-5-(4-(6-((4-isopropylphenyl)carbamoyl)-2-oxa-6-azaspiro[3.3]heptane-5-carboxamido)phenyl)-6-methylpicolinic acid and (R)-5-(4-(6-((4-isopropylphenyl)carbamoyl)-2-oxa-6-azaspiro[3.3]heptane-5-carboxamido)phenyl)-6-methylpicolinic acid

### Step 1: 2-oxa-6-azaspiro[3.3]heptane-5-carboxylic acid

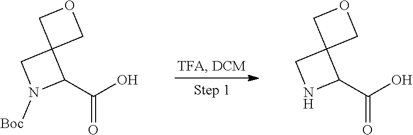 [SMILES: CC(C)(C)OC(=O)N1CC2(COC2)C1C(=O)O ; O=C(O)C1NCC12COC2]

A solution of 6-(tert-butoxycarbonyl)-2-oxa-6-azaspiro[3.3]heptane-5-carboxylic acid (90.0 mg, 0.370 mmol) and TFA (2 mL) in DCM (2 mL) was stirred at room temperature for 1 hour. Upon completion, the reaction mixture was concentrated under reduced pressure to afford 2-oxa-6-azaspiro[3.3]heptane-5-carboxylic acid (55 mg, crude product) as a yellow oil, which was used directly in the next step without further purification. LCMS (ESI) [M+H]⁺: 144.1.

### Step 2: 6-((4-isopropylphenyl)carbamoyl)-2-oxa-6-azaspiro[3.3]heptane-5-carboxylic acid

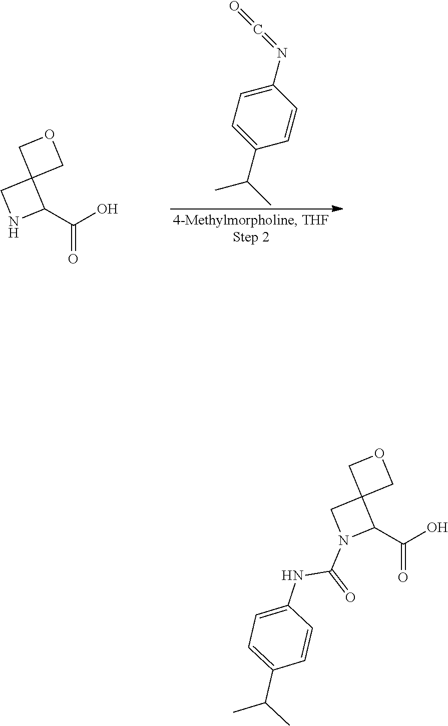 [SMILES: O=C(O)C1NCC12COC2 ; CC(C)c1ccc(N=C=O)cc1 ; CC(C)c1ccc(NC(=O)N2CC3(COC3)C2C(=O)O)cc1]

A solution of 1-isocyanato-4-isopropylbenzene (124 mg, 0.768 mmol), 2-oxa-6-azaspiro[3.3]heptane-5-carboxylic acid (55.0 mg, 0.384 mmol) and 4-methylmorpholine (117 mg, 1.15 mmol) in THF (3 mL) was stirred at 25° C. for 1 hour. Upon completion, the reaction mixture was directly purified by reversed-phase flash chromatography (conditions: column: C18 silica gel; mobile phase A: water (0.1% formic acid); mobile phase B: MeCN; gradient (B %): 0% to 100% over 30 min; detector: UV 254 nm) to afford 6-((4-isopropylphenyl)carbamoyl)-2-oxa-6-azaspiro[3.3]heptane-5-carboxylic acid (71 mg, 61%) as a white solid. LCMS (ESI) [M+H]⁺: 305.1.

### Step 3: methyl 5-(4-(6-((4-isopropylphenyl)carbamoyl)-2-oxa-6-azaspiro[3.3]heptane-5-carboxamido)phenyl)-6-methylpicolinate

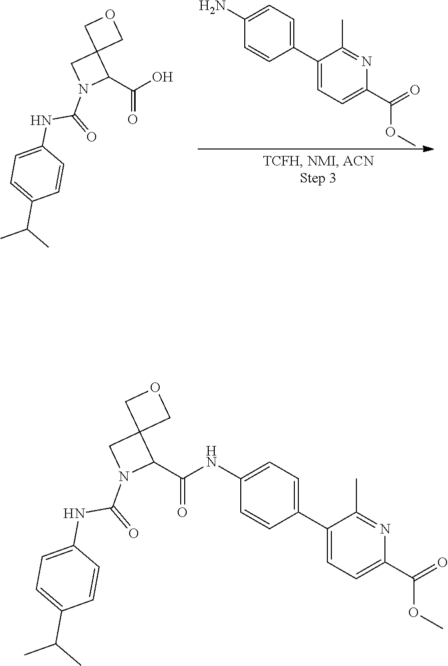 [SMILES: CC(C)c1ccc(NC(=O)N2CC3(COC3)C2C(=O)O)cc1 ; COC(=O)c1ccc(-c2ccc(N)cc2)c(C)n1 ; COC(=O)c1ccc(-c2ccc(NC(=O)C3N(C(=O)Nc4ccc(C(C)C)cc4)CC34COC4)cc2)c(C)n1]

A solution of methyl 5-(4-aminophenyl)-6-methylpicolinate (83.6 mg, 0.345 mmol), 1-methylimidazole (94.4 mg, 1.15 mmol), TCFH (129 mg, 0.460 mmol) and 6-((4-isopropylphenyl)carbamoyl)-2-oxa-6-azaspiro[3.3]heptane-5-carboxylic acid (70 mg, 0.230 mmol) in ACN (3 mL) was stirred at 25° C. for 1 hour. Upon completion, the reaction mixture was directly purified by reversed-phase flash chromatography (conditions: column: C18 silica gel; mobile phase A: water (0.1% formic acid); mobile phase B: MeCN; gradient (B %): 0% to 100% over 30 min; detector: UV 254 nm) to afford methyl 5-(4-(6-((4-isopropylphenyl)carbamoyl)-2-oxa-6-azaspiro[3.3]heptane-5-carboxamido)phenyl)-6-methylpicolinate (60 mg, 49%) as a white solid. LCMS (ESI) [M+H]⁺: 529.2.

### Step 4: 5-(4-(6-((4-isopropylphenyl)carbamoyl)-2-oxa-6-azaspiro[3.3]heptane-5-carboxamido)phenyl)-6-methylpicolinic acid

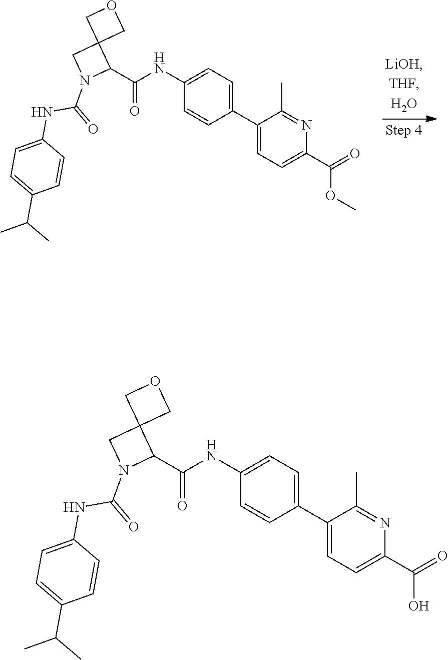

A solution of methyl 5-(4-(6-((4-isopropylphenyl)carbamoyl)-2-oxa-6-azaspiro[3.3]heptane-5-carboxamido)phenyl)-6-methylpicolinate (60.0 mg, 0.114 mmol) and lithium hydroxide monohydrate (23.9 mg, 0.570 mmol) in THF (2 mL) and water (0.5 mL) was stirred at 25° C. for 1 hour. Upon completion, the reaction mixture was directly purified by reversed-phase flash chromatography (conditions: column: C18 silica gel; mobile phase A: water (0.1% formic acid); mobile phase B: MeCN; gradient (B %): 0% to 100% over 30 min; detector: UV 254 nm) to afford 5-(4-(6-((4-isopropylphenyl)carbamoyl)-2-oxa-6-azaspiro[3.3]heptane-5-carboxamido)phenyl)-6-methylpicolinic acid (30 mg, 51%) as a white solid. LCMS (ESI) [M+H]⁺: 515.2.

### Step 5: (S)-5-(4-(6-((4-isopropylphenyl)carbamoyl)-2-oxa-6-azaspiro[3.3]heptane-5-carboxamido)phenyl)-6-methylpicolinic acid and (R)-5-(4-(6-((4-isopropylphenyl)carbamoyl)-2-oxa-6-azaspiro[3.3]heptane-5-carboxamido)phenyl)-6-methylpicolinic acid

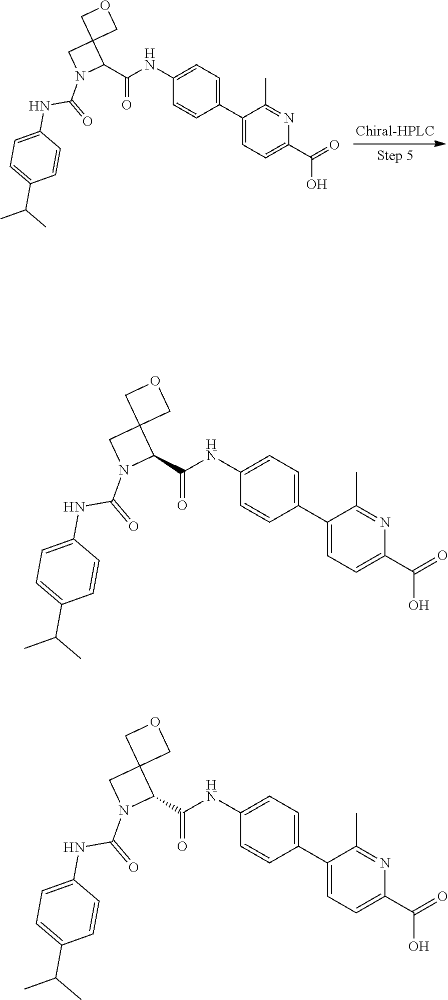 [SMILES: Cc1nc(C(=O)O)ccc1-c1ccc(NC(=O)C2N(C(=O)Nc3ccc(C(C)C)cc3)CC23COC3)cc1 ; Cc1nc(C(=O)O)ccc1-c1ccc(NC(=O)[C@H]2N(C(=O)Nc3ccc(C(C)C)cc3)CC23COC3)cc1 ; Cc1nc(C(=O)O)ccc1-c1ccc(NC(=O)[C@@H]2N(C(=O)Nc3ccc(C(C)C)cc3)CC23COC3)cc1]

5-(4-(6-((4-isopropylphenyl)carbamoyl)-2-oxa-6-azaspiro[3.3]heptane-5-carboxamido)phenyl)-6-methylpicolinic acid (30 mg) was resolved by preparative chiral HPLC (conditions: Column: CHIRAL ART Cellulose-SB, 3*25 cm, 5 μm; Mobile Phase A: Hexanes (0.1% FA), Mobile Phase B: EtOH; Flow rate: 40 mL/min; Gradient (B %): isocratic, 50% B; Wave Length: 220/254 nm; RT1 (min): 9.22; RT2 (min): 15.99) to afford (Compound 321a) (S)-5-(4-(6-((4-isopropylphenyl)carbamoyl)-2-oxa-6-azaspiro[3.3]heptane-5-carboxamido)phenyl)-6-methylpicolinic acid (1^{st} peak, 7.0 mg, 23%; Method BI, Peak 1, 1.39 min) as a white solid and (Compound 321b) (R)-5-(4-(6-((4-isopropylphenyl)carbamoyl)-2-oxa-6-azaspiro[3.3]heptane-5-carboxamido)phenyl)-6-methylpicolinic acid (2^{nd} peak, 7.9 mg, 26%; Method BI, Peak 2, 2.82 min) as a white solid.

| Cmpd # | Characterization Data |
|---|---|
| 321a | LCMS (ESI) [M + H]⁺: 515.3 |
| | ¹H NMR (400 MHz, Methanol-d₄) δ 8.05 (d, J = 7.9 Hz, 1H), 7.85 (d, J = 7.8 Hz, 1H), |
| | 7.81 (d, J = 8.3 Hz, 2H), 7.40 (d, J = 8.3 Hz, 2H), 7.37-7.28 (m, 2H), 7.19-7.08 (m, |
| | 2H), 4.96 (d, J = 7.2 Hz, 2H), 4.83 (s, 1H), 4.73 (q, J = 7.5 Hz, 2H), 4.36 (d, J = 8.4 |
| | Hz, 1H), 4.31 (d, J = 8.4 Hz, 1H), 2.83 (h, J = 7.0 Hz, 1H), 2.57 (s, 3H), 1.21 (d, J = |
| | 6.9 Hz, 6H). |
| 321b | LCMS (ESI) [M + H]⁺: 515.3 |
| | ¹H NMR (400 MHz, Methanol-d₄) δ 8.07 (d, J = 7.8 Hz, 1H), 7.85 (d, J = 7.8 Hz, 1H), |
| | 7.80 (d, J = 8.1 Hz, 2H), 7.38 (d, J = 8.0 Hz, 2H), 7.33 (d, J = 8.3 Hz, 2H), 7.13 (d, J = |
| | 8.7 Hz, 2H), 4.96 (d, J = 7.2 Hz, 2H), 4.84 (s, 1H), 4.73 (q, J = 7.5 Hz, 2H), 4.39- |
| | 4.27 (m, 2H), 2.83 (h, J = 6.8 Hz, 1H), 2.56 (s, 3H), 1.21 (d, J = 7.0 Hz, 6H). |

### Example 322: (S)-2-(5-(1H-[1,2,3]triazolo[4,5-b]pyridin-5-yl)-3-fluoropyridin-2-yl)-N-(2,2-difluoro-[1,3]dioxolo[4,5-b]pyridin-5-yl)pyrrolidine-1-carboxamide and (R)-2-(5-(1H-[1,2,3]triazolo[4,5-b]pyridin-5-yl)-3-fluoropyridin-2-yl)-N-(2,2-difluoro-[1,3]dioxolo[4,5-b]pyridin-5-yl)pyrrolidine-1-carboxamide

### Step 1: N-(2,2-difluoro-[1,3]dioxolo[4,5-b]pyridin-5-yl)-2-(3-fluoro-5-(1-((2-(trimethylsilyl)ethoxy)methyl)-1H-[1,2,3]triazolo[4,5-b]pyridin-5-yl)pyridin-2-yl)pyrrolidine-1-carboxamide

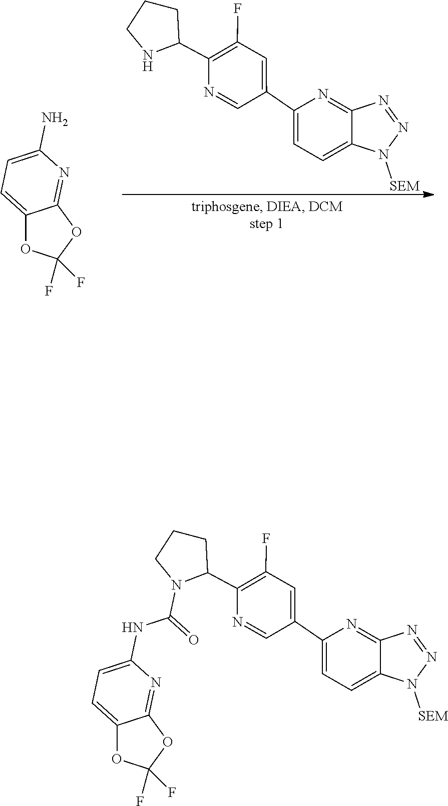 [SMILES: Nc1ccc2c(n1)OC(F)(F)O2 ; C[Si](C)(C)CCOCn1nnc2nc(-c3cnc(C4CCCN4)c(F)c3)ccc21 ; C[Si](C)(C)CCOCn1nnc2nc(-c3cnc(C4CCCN4C(=O)Nc4ccc5c(n4)OC(F)(F)O5)c(F)c3)ccc21]

A solution of 2,2-difluoro-[1,3]dioxolo[4,5-b]pyridin-5-amine (130 mg, 0.747 mmol), DIEA (96.5 mg, 0.747 mmol), 5-(5-fluoro-6-(pyrrolidin-2-yl)pyridin-3-yl)-1-((2-(trimethylsilyl)ethoxy)methyl)-1H-[1,2,3]triazolo[4,5-b]pyridine (309 mg, 0.747 mmol) and triphosgene (88.6 mg, 0.299 mmol) in DCM (2 mL) was stirred at 25° C. for 1 hour. Upon completion, the resulting mixture was concentrated under reduced pressure. The crude residue was directly purified by silica gel column chromatography, eluted with petroleum ether/ethyl acetate (1:1) to afford N-(2,2-difluoro-[1,3]dioxolo[4,5-b]pyridin-5-yl)-2-(3-fluoro-5-(1-((2-(trimethylsilyl)ethoxy)methyl)-1H-[1,2,3]triazolo[4,5-b]pyridin-5-yl)pyridin-2-yl)pyrrolidine-1-carboxamide (100 mg, 21%) as a yellow oil. LCMS (ESI) [M+H]⁺: 615.2.

### Step 2: 2-(5-(1H-[1,2,3]triazolo[4,5-b]pyridin-5-yl)-3-fluoropyridin-2-yl)-N-(2,2-difluoro-[1,3]dioxolo[4,5-b]pyridin-5-yl)pyrrolidine-1-carboxamide

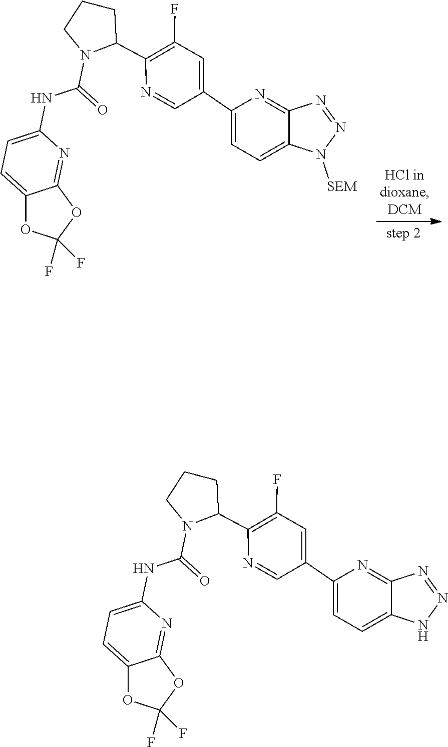 [SMILES: C[Si](C)(C)CCOCn1nnc2nc(-c3cnc(C4CCCN4C(=O)Nc4ccc5c(n4)OC(F)(F)O5)c(F)c3)ccc21 ; O=C(Nc1ccc2c(n1)OC(F)(F)O2)N1CCCC1c1ncc(-c2ccc3[nH]nnc3n2)cc1F]

HCl (1 mL, 4 mmol, 4.0 M in 1,4-dioxane) was added into a solution of N-(2,2-difluoro-[1,3]dioxolo[4,5-b]pyridin-5-yl)-2-(3-fluoro-5-(1-((2-(trimethylsilyl)ethoxy)methyl)-1H-[1,2,3]triazolo[4,5-b]pyridin-5-yl)pyridin-2-yl)pyrrolidine-1-carboxamide (100 mg, 0.163 mmol) in DCM (1 mL) and the resulting mixture was stirred at 25° C. for 1 hour. Upon completion, the mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 30 min; detector: UV 254 nm) to afford 2-(5-(1H-[1,2,3]triazolo[4,5-b]pyridin-5-yl)-3-fluoropyridin-2-yl)-N-(2,2-difluoro-[1,3]dioxolo[4,5-b]pyridin-5-yl)pyrrolidine-1-carboxamide (60 mg, 76%) as a white solid. LCMS (ESI) [M+H]⁺: 485.1.

### Step 3: (S)-2-(5-(1H-[1,2,3]triazolo[4,5-b]pyridin-5-yl)-3-fluoropyridin-2-yl)-N-(2,2-difluoro-[1,3]dioxolo[4,5-b]pyridin-5-yl)pyrrolidine-1-carboxamide and (R)-2-(5-(1H-[1,2,3]triazolo[4,5-b]pyridin-5-yl)-3-fluoropyridin-2-yl)-N-(2,2-difluoro-[1,3]dioxolo[4,5-b]pyridin-5-yl)pyrrolidine-1-carboxamide

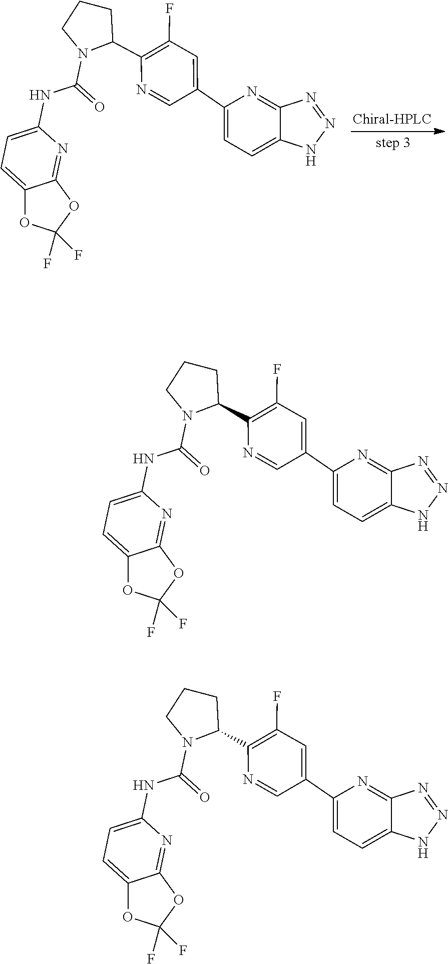 [SMILES: O=C(Nc1ccc2c(n1)OC(F)(F)O2)N1CCCC1c1ncc(-c2ccc3[nH]nnc3n2)cc1F ; O=C(Nc1ccc2c(n1)OC(F)(F)O2)N1CCC[C@H]1c1ncc(-c2ccc3[nH]nnc3n2)cc1F ; O=C(Nc1ccc2c(n1)OC(F)(F)O2)N1CCC[C@@H]1c1ncc(-c2ccc3[nH]nnc3n2)cc1F]

2-(5-(1H-[1,2,3]triazolo[4,5-b]pyridin-5-yl)-3-fluoropyridin-2-yl)-N-(2,2-difluoro-[1,3]dioxolo[4,5-b]pyridin-5-yl)pyrrolidine-1-carboxamide (60 mg) was resolved by preparative chiral HPLC (conditions: Column: CHIRAL ART Cellulose-SZ, 3*25 cm, 5 μm; Mobile Phase A: Hexanes (0.1% FA), Mobile Phase B: ETOH; Flow rate: 40 mL/min; Gradient (B %): isocratic, 30% B; Wave Length: 220/254 nm; RT1 (min): 12.5; RT2 (min): 15.5) to afford (Compound 322a) (S)-2-(5-(1H-[1,2,3]triazolo[4,5-b]pyridin-5-yl)-3-fluoropyridin-2-yl)-N-(2,2-difluoro-[1,3]dioxolo[4,5-b]pyridin-5-yl)pyrrolidine-1-carboxamide (1^{st} peak, 11.1 mg, 18%; Method BM, Peak 1, 1.53 min) as a white solid and (Compound 322b) (R)-2-(5-(1H-[1,2,3]triazolo[4,5-b]pyridin-5-yl)-3-fluoropyridin-2-yl)-N-(2,2-difluoro-[1,3]dioxolo[4,5-b]pyridin-5-yl)pyrrolidine-1-carboxamide (2^{nd} peak, 27.8 mg, 47%; Method BM, Peak 2, 1.96 min) as a white solid.

| Cmpd # | Characterization Data |
|---|---|
| 322a | LCMS (ESI) [M + H]⁺: 485.1 |
| | ¹H NMR (400 MHz, Methanol-d₄) δ 9.10 (t, J = 1.4 Hz, 1H), 8.45 (d, J = 8.7 Hz, 1H), 8.34 |
| | (dd, J = 11.3, 1.8 Hz, 1H), 8.10 (d, J = 8.7 Hz, 1H), 7.56 (d, J = 8.7 Hz, 1H), 7.47 (d, J = 8.7 |
| | Hz, 1H), 5.49 (dd, J = 8.1, 4.3 Hz, 1H), 3.95-3.83 (m, 1H), 3.80-3.67 (m, 1H), 2.49 (dd, J = |
| | 12.6, 7.3 Hz, 1H), 2.28-2.03 (m, 3H). |
| 322b | LCMS (ESI) [M + H]⁺: 485.0 |
| | ¹H NMR (400 MHz, Methanol-d₄) δ 9.10 (s, 1H), 8.46 (d, J = 8.7 Hz, 1H), 8.34 (dd, J = 11.3, |
| | 1.8 Hz, 1H), 8.10 (d, J = 8.6 Hz, 1H), 7.56 (d, J = 8.7 Hz, 1H), 7.47 (d, J = 8.8 Hz, 1H), 5.49 |
| | (dd, J = 8.2, 4.1 Hz, 1H), 3.94-3.83 (m, 1H), 3.79-3.69 (m, 1H), 2.48 (dt, J = 15.1, 7.6 Hz, |
| | 1H), 2.14 (ddq, J = 31.9, 19.8, 6.2 Hz, 3H). |

### Example 323: (S)-2-(5-(1H-[1,2,3]triazolo[4,5-b]pyridin-5-yl)-3-fluoropyridin-2-yl)-N-(4-methyl-5-(1-(trifluoromethyl)cyclopropyl)pyridin-2-yl)pyrrolidine-1-carboxamide and (R)-2-(5-(1H-[1,2,3]triazolo[4,5-b]pyridin-5-yl)-3-fluoropyridin-2-yl)-N-(4-methyl-5-(1-(trifluoromethyl)cyclopropyl)pyridin-2-yl)pyrrolidine-1-carboxamide

### Step 1: 2-(3-fluoro-5-(1-((2-(trimethylsilyl)ethoxy)methyl)-1H-[1,2,3]triazolo[4,5-b]pyridin-5-yl)pyridin-2-yl)-N-(4-methyl-5-(1-(trifluoromethyl)cyclopropyl)pyridin-2-yl)pyrrolidine-1-carboxamide

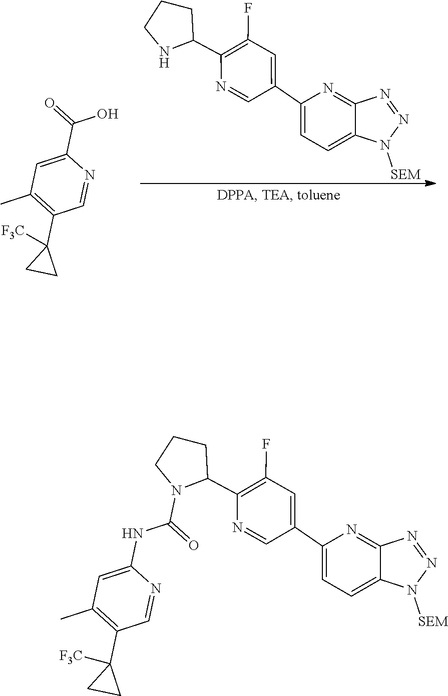 [SMILES: Cc1cc(C(=O)O)ncc1C1(C(F)(F)F)CC1 ; C[Si](C)(C)CCOCn1nnc2nc(-c3cnc(C4CCCN4)c(F)c3)ccc21 ; Cc1cc(NC(=O)N2CCCC2c2ncc(-c3ccc4c(nnn4COCC[Si](C)(C)C)n3)cc2F)ncc1C1(C(F)(F)F)CC1]

A solution of 4-methyl-5-[1-(trifluoromethyl)cyclopropyl]pyridine-2-carboxylic acid (100 mg, 0.408 mmol), DPPA (336 mg, 1.22 mmol), 5-(5-fluoro-6-(pyrrolidin-2-yl)pyridin-3-yl)-1-((2-(trimethylsilyl)ethoxy)methyl)-1H-[1,2,3]triazolo[4,5-b]pyridine (186 mg, 0.449 mmol) and triethylamine (124 mg, 1.22 mmol) in toluene (5.0 mL) was stirred at 80° C. for 1 hour. Upon completion, the resulting mixture was concentrated under reduced pressure. The crude residue was directly purified by silica gel column chromatography, eluted with petroleum ether/ethyl acetate (1:1) to afford 2-(3-fluoro-5-(1-((2-(trimethylsilyl)ethoxy)methyl)-1H-[1,2,3]triazolo[4,5-b]pyridin-5-yl)pyridin-2-yl)-N-(4-methyl-5-(1-(trifluoromethyl)cyclopropyl)pyridin-2-yl)pyrrolidine-1-carboxamide (90 mg, 34%) as a yellow solid. LCMS (ESI) [M+H]⁺: 657.2

### Step 2: 2-(5-(1H-[1,2,3]triazolo[4,5-b]pyridin-5-yl)-3-fluoropyridin-2-yl)-N-(4-methyl-5-(1-(trifluoromethyl)cyclopropyl)pyridin-2-yl)pyrrolidine-1-carboxamide

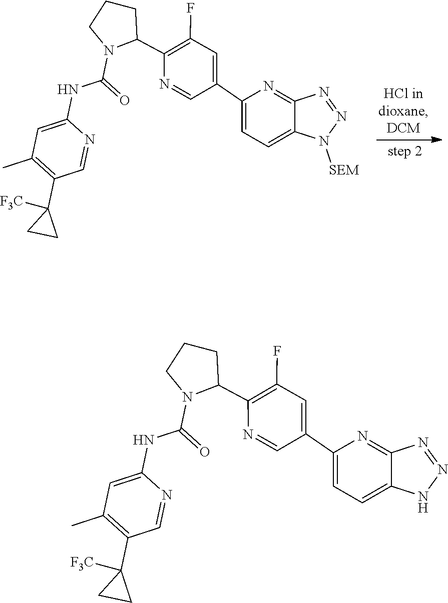 [SMILES: Cc1cc(NC(=O)N2CCCC2c2ncc(-c3ccc4c(nnn4COCC[Si](C)(C)C)n3)cc2F)ncc1C1(C(F)(F)F)CC1 ; Cc1cc(NC(=O)N2CCCC2c2ncc(-c3ccc4[nH]nnc4n3)cc2F)ncc1C1(C(F)(F)F)CC1]

HCl (1 mL, 4 mmol, 4.0 M in 1,4-dioxane) was added into a solution of 2-(3-fluoro-5-(1-((2-(trimethylsilyl)ethoxy)methyl)-1H-[1,2,3]triazolo[4,5-b]pyridin-5-yl)pyridin-2-yl)-N-(4-methyl-5-(1-(trifluoromethyl)cyclopropyl)pyridin-2-yl)pyrrolidine-1-carboxamide (90 mg, 0.137 mmol) in DCM (1 mL) and the resulting mixture was stirred at 25° C. for 1 hour. Upon completion, the mixture was concentrated under reduced pressure. The crude residue was directly purified by reversed-phase flash chromatography (conditions: column: C18 silica gel; mobile phase A: water (0.1% formic acid); mobile phase B: MeCN; gradient (B %): 0% to 100% over 30 min; detector: UV 254 nm) to afford 2-(5-(1H-[1,2,3]triazolo[4,5-b]pyridin-5-yl)-3-fluoropyridin-2-yl)-N-(4-methyl-5-(1-(trifluoromethyl)cyclopropyl)pyridin-2-yl)pyrrolidine-1-carboxamide (60 mg, 83%) as a white solid. LCMS (ESI) [M+H]⁺: 527.3.

### Step 3: (S)-2-(5-(1H-[1,2,3]triazolo[4,5-b]pyridin-5-yl)-3-fluoropyridin-2-yl)-N-(4-methyl-5-(1-(trifluoromethyl)cyclopropyl)pyridin-2-yl)pyrrolidine-1-carboxamide and (R)-2-(5-(1H-[1,2,3]triazolo[4,5-b]pyridin-5-yl)-3-fluoropyridin-2-yl)-N-(4-methyl-5-(1-(trifluoromethyl)cyclopropyl)pyridin-2-yl)pyrrolidine-1-carboxamide

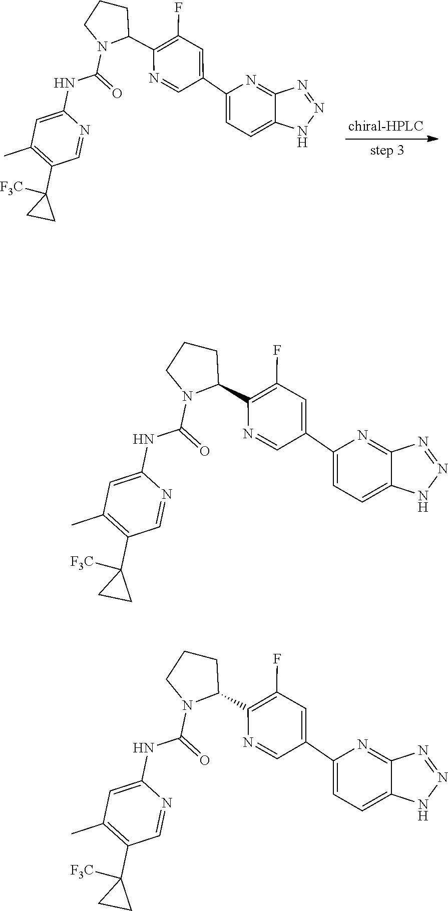 [SMILES: Cc1cc(NC(=O)N2CCCC2c2ncc(-c3ccc4[nH]nnc4n3)cc2F)ncc1C1(C(F)(F)F)CC1 ; Cc1cc(NC(=O)N2CCC[C@H]2c2ncc(-c3ccc4[nH]nnc4n3)cc2F)ncc1C1(C(F)(F)F)CC1 ; Cc1cc(NC(=O)N2CCC[C@@H]2c2ncc(-c3ccc4[nH]nnc4n3)cc2F)ncc1C1(C(F)(F)F)CC1]

2-(5-(1H-[1,2,3]triazolo[4,5-b]pyridin-5-yl)-3-fluoropyridin-2-yl)-N-(4-methyl-5-(1-(trifluoromethyl)cyclopropyl)pyridin-2-yl)pyrrolidine-1-carboxamide (60 mg) was resolved by preparative chiral HPLC (conditions: Column: Lux 5 μm Cellulose-2 3*25 cm, 5 μm; Mobile Phase A: Hexanes (0.1% FA), Mobile Phase B: EtOH; Flow rate: 40 mL/min; Gradient (B %): isocratic, 50% B; Wave Length: 220/254 nm; RT1 (min): 10.7; RT2 (min): 14.8) to afford (Compound 323a) (S)-2-(5-(1H-[1,2,3]triazolo[4,5-b]pyridin-5-yl)-3-fluoropyridin-2-yl)-N-(4-methyl-5-(1-(trifluoromethyl)cyclopropyl)pyridin-2-yl)pyrrolidine-1-carboxamide (1^{st} peak, 14.6 mg, 24%; Method BN, Peak 1, 1.31 min) as a white solid and (Compound 323b) (R)-2-(5-(1H-[1,2,3]triazolo[4,5-b]pyridin-5-yl)-3-fluoropyridin-2-yl)-N-(4-methyl-5-(1-(trifluoromethyl)cyclopropyl)pyridin-2-yl)pyrrolidine-1-carboxamide (2^{nd} peak, 4.0 mg, 7%; Method BN, Peak 2, 1.66 min) as a white solid.

| Cmpd # | Characterization Data |
|---|---|
| 323a | LCMS (ESI) [M + H]⁺: 527.2 |
| | ¹H NMR (400 MHz, DMSO-d₆) δ 9.09 (d, J = 2.0 Hz, 1H), 8.83 (s, 1H), 8.42-8.29 (m, 2H), |
| | 8.14 (s, 1H), 7.95 (d, J = 8.6 Hz, 1H), 7.67 (s, 1H), 5.39 (s, 1H), 3.88-3.77 (m, 1H), 3.74- |
| | 3.64 (m, 1H), 2.40-2.25 (m, 4H), 2.16-1.94 (m, 3H), 1.38 (s, 2H), 1.10 (s, 2H). |
| 323b | LCMS (ESI) [M + H]⁺: 527.2 |
| | ¹H NMR (400 MHz, DMSO-d₆) δ 9.10 (s, 1H), 8.82 (s, 1H), 8.42 (d, J = 8.6 Hz, 1H), 8.34 (d, J = |
| | 11.6 Hz, 1H), 8.14 (s, 1H), 8.00 (d, J = 8.5 Hz, 1H), 7.67 (s, 1H), 5.39 (bs, 1H), 3.86-3.78 |
| | (m, 1H), 3.72-3.64 (m, 1H), 2.42-2.24 (m, 4H), 2.17-1.87 (m, 3H), 1.38 (s, 2H), 1.10 (s, 2H). |

### Biological Examples

The utility of the compounds of the present invention and the salts of such compounds as medical agents in the treatment of the above described disease/conditions in mammals (e.g. humans, male or female) is demonstrated by the activity of the compounds of the present invention in one or more of the conventional assays noted below. Thus, the protocols described below can also be used to demonstrate the utility of the combinations of the compounds of the present invention. The assays and models may also demonstrate particular other property advantages e.g., side effect profile; half-life. In addition, such assays provide a means whereby the activities of the compounds of the present invention and the salts of such compounds (or the other agents described herein) can be compared to each other and with the activities of other known compounds. The results of these comparisons are useful for determining dosage levels in mammals, including humans, for the treatment of such diseases.

Absorption, Distribution, Metabolism and Excretion (ADME) and pharmacokinetics (PK) of compounds and exemplary assays are discussed in the on-line publication by Thomas D. Y. Chung, David B. Terry and Layton H. Smith “In Vitro and In Vivo Assessment of ADME and PK Properties During Lead Selection and Lead Optimization—Guidelines, Benchmarks and Rules of Thumb—(https://www.ncbi.nlm.nih.gov/books/NBK326710/)

Exemplary in vitro and in vivo models of obesity are described in “A Review on in-vivo and in-vitro Models of Obesity and Obesity-Associated Co-Morbidities” Digbijoy Nath, Pervej Alom Barbhuiya, Saikat Sen, Manash Pratim Pathak; Endocr Metab Immune Disord Drug Targets (2024 Aug. 12).

Exemplary in vivo models of autoimmune disease/conditions are included in Autoimmun Rev. 2018 May; 17(5): 473-479. Exemplary in vitro assays of cancer are described in Front Bioeng Biotechnol 2016; 4:12 “In Vitro Tumore Models: Advantages, Disadvantages, Variables, and Selecting the Right Platform.” Exemplary in vivo animal models of cancer are described in Front Oncol, 2019; 8: 429 “Next-Generation in vivo Modeling of Human Cancers. Exemplary in vivo animal models of pain are described in Journal of Neuroscience Methods Volume 348, January 2021, 108997, “Animal models of pain: Diversity and benefits” and J. Pain. 2013 November: 14(11): 10.1016/j.jpain.2013.06.008 “An overview of animal models of pain: disease models and outcome measures”.

The following protocols may of course be varied by those skilled in art.

Protocol 1: hGIPR Antagonist Assay:

hGIPR/CHO-K1 cell line stably transfected to express human GIPR (gene of interest) were maintained at 37° C. with 5% CO₂ and grown in Ham's F12 media supplemented with 10% FBS, 1% penicillin/streptomycin, and 10 μg/mL of puromycin.

On the day of experiment, cells were harvested and counted using a Countess II cell counter; only cells with viability >90% were used for assays. Cells were diluted to 1E⁵ cells/mL with cAMP assay buffer (1×HBSS with Ca²⁺ and Mg²+, 20 mM HEPES, 0.1% BSA, and 500 μM IBMX). Compounds were transferred into 384-well assay plates in determined concentration response range, with constant percentage of DMSO below 0.5%, 10 μL of cells were then seeded into plates, and briefly centrifuged for 1 min at 600 rpm. Compounds were allowed to incubate with cells for 30 min at 25° C., after which assay buffer containing GIP (Gastric inhibitory polypeptide; G2269-0.1MG, Sigma-Aldrich) at EC₈₀ was added to each well. Assay plate was once again briefly centrifuged for 1 min at 600 rpm, and then incubated for an additional 30 min at 25° C. Detection reagent was the LANCE Ultra cAMP kit from Revvity (TRF0264). 5 μL/well of Eu-cAMP tracer working solution and 5 μL/well of anti-cAMP working solution were added to each well and assay plate was centrifuged for 1 min at 600 rpm. Assay plate was allowed to incubate for 60 min at room temperature, and results were read with EnVision microplate reader ((λex=337 nm, λem=615 nm and 665 nm). The HTRF ratio was calculated from raw data (channel 1/channel 2×10,000), concentration response curve was plotted and IC₅₀ was calculated with 4 parameter nonlinear regression model.

### Protocol 1: HGCGR Antagonist Assay:

hGCGR/Flp in CHO-K1 cell line stably transfected to express human GCGR (gene of interest) were maintained at 37° C. with 5% CO₂ and grown in Ham's F12 media supplemented with 10% FBS, 1% penicillin/streptomycin, and 800 μg/mL of Hygromycin B.

On the day of experiment, cells were harvested and counted using a Countess II cell counter; only cells with viability >90% were used for assays. Cells were diluted to 2E⁵ cells/mL with cAMP assay buffer (1×HBSS with Ca²⁺ and Mg²+, 20 mM HEPES, 0.1% BSA, and 500 μM IBMX). Compounds were transferred into 384-well assay plates in determined concentration response range, with constant percentage of DMSO below 0.5%, 10 μL of cells were then seeded into plates, and briefly centrifuged for 1 min at 600 rpm. Compounds were allowed to incubate with cells for 30 min at 25° C., after which assay buffer containing glucagon at EC₈₀ was added to each well. Assay plate was once again briefly centrifuged for 1 min at 600 rpm, and then incubated for an additional 30 min at 25° C. Detection reagent was the LANCE Ultra cAMP kit from Revvity (TRF0264). 5 μL/well of Eu-cAMP tracer working solution and 5 μL/well of anti-cAMP working solution were added to each well and assay plate was centrifuged for 1 min at 600 rpm. Assay plate was allowed to incubate for 60 min at room temperature, and results were read with EnVision microplate reader ((λex=337 nm, λem=615 nm and 665 nm). The HTRF ratio was calculated from raw data (channel 1/channel 2×10,000), concentration response curve was plotted and IC₅₀ was calculated with 4 parameter nonlinear regression model. Table 5 shows compound activity characterized in hGIPR antagonist assay and hGCGR antagonist assay

### Protocol 2: HGIPR Antagonist Potency Assay:

hGIPR/CHO-K1 cell line (Eurofins, DiscoverX, Cat No. 95-0146C2) stably transfected to express human GIPR (gene of interest) were maintained at 37° C. with 5% CO₂ and grown in Eurofins Assay Complete Cell Culture Kit 107+800 μg/mL G418. Assay ready cells were expanded, frozen and aliquoted into screen ready vials at 1×10⁷ cells/vial.

Test articles were solubilized in 100% DMSO as 30 mM stock solutions, 11-point serial dilution was then generated using 1:4 dilution in 100% DMSO, top concentration of 8 mM in ‘serial dilution plate’. On day of experiment, cells were thawed and counted using a Countess II cell counter; only cells with viability >90% were used for assays. Assay cells were diluted to 4×10⁵ cells/mL with cAMP assay buffer (1×HBSS with Ca²⁺ and Mg²+, 20 mM HEPES, 0.1% BSA, and 200 μM IBMX). Test articles from serial dilution plate were transferred into 384-well assay plates in determined concentration response range, with constant percentage of DMSO below 0.5%, 10 μL of cells were then seeded into plates and briefly centrifuged for 1 minute at 600 rpm. Test articles were allowed to incubate with cells for 2 hours at 37° C., after which assay buffer containing GIP (Gastric inhibitory polypeptide; G2269-0.1MG, Sigma-Aldrich) at EC₈₀ was added to each well. Assay plate was once again briefly centrifuged for 1 minute at 600 rpm, and then incubated for an additional 30 minutes at 37° C. Detection reagent was the dynamic cAMP Revvity (62AM4PEB). 5 μL/well of Eu-cAMP tracer working solution and 5 μL/well of anti-cAMP working solution were added to each well and assay plate was centrifuged for 1 minute at 600 rpm. Assay plate was allowed to incubate for 60 minute at room temperature, and results were read with EnVision microplate reader ((λex=337 nm, λem=615 nm and 665 nm). The HTRF ratio was calculated from raw data (channel 1/channel 2×10,000), converted to cAMP concentration according to standard curve and concentration response curve was plotted with IC₅₀ determined using 4 parameter nonlinear regression model. Data was then normalized to EC₈₀ of GIP as 100% and DMSO/vehicle control as 0%. Experiment passed quality control if constant concentration of GIP used fell within EC₅₀ and EC₉₀ of the agonist response curve determined on day of experiment.

Protocol 2: hGCGR Antagonist Potency Assay

hGCGR/CHO-K1 cell line (Eurofins, DiscoverX, Cat No. 95-0042C2) stably transfected to express human GCGR (gene of interest) were maintained at 37° C. with 5% CO₂ and grown in Eurofins Assay Complete Cell Culture Kit 107+800 μg/mL G418. Assay ready cells were expanded, frozen and aliquoted into screen ready vials at 1×10⁷ cells/vial.

Test articles were solubilized in 100% DMSO as 30 mM stock solutions, 11-point serial dilution was then generated using 1:4 dilution in 100% DMSO, top concentration of 8 mM in ‘serial dilution plate’. On day of experiment, cells were thawed and counted using a Countess II cell counter; only cells with viability >90% were used for assays. Assay cells were diluted to 4×10⁵ cells/mL with cAMP assay buffer (1×HBSS with Ca²⁺ and Mg²+, 20 mM HEPES, 0.1% BSA, and 200 μM IBMX). Test articles from serial dilution plate were transferred into 384-well assay plates in determined concentration response range, with constant percentage of DMSO below 0.5%, 10 μL of cells were then seeded into plates and briefly centrifuged for 1 minute at 600 rpm. Test articles were allowed to incubate with cells for 2 hours at 37° C., after which assay buffer containing glucagon at EC₈₀ was added to each well. Assay plate was once again briefly centrifuged for 1 minute at 600 rpm, and then incubated for an additional 30 minutes at 37 C. Detection reagent was the dynamic cAMP Revvity (62AM4PEB). 5 μL/well of Eu-cAMP tracer working solution and 5 μL/well of anti-cAMP working solution were added to each well and assay plate was centrifuged for 1 minute at 600 rpm. Assay plate was allowed to incubate for 60 minutes at room temperature, and results were read with EnVision microplate reader ((λex=337 nm, λem=615 nm and 665 nm). The HTRF ratio was calculated from raw data (channel 1/channel 2×10,000), converted to cAMP concentration according to standard curve and concentration response curve was plotted with IC₅₀ determined using 4 parameter nonlinear regression model. Data was then normalized to EC₈₀ of glucagon as 100% and DMSO/vehicle control as 0%. Experiment passed quality control if constant concentration of GIP used fell within EC₅₀ and EC₉₀ of the agonist response curve determined on day of experiment.

Table 5 and Table 6 show compound activity characterized in hGIPR antagonist assays and hGCGR antagonist assays detailed above. A blank cell or “N/A” indicates the compound was not tested in that assay.

TABLE 5
Assay Results from Protocol 1

| | | hGIPR % | | hGCGR % |
|---|---|---|---|---|
| | hGIPR | Inhibition | hGCGR | Inhibition |
| Cmpd # | IC₅₀ (nM) | at 10 μM | IC₅₀ (nM) | at 10 μM |
| 6 | >10000 | 22.7 | >10000 | 7.7 |
| 7 | >10000 | 9.7 | >10000 | 4.9 |
| 8 | >10000 | 19.6 | >10000 | 7.2 |
| 9 | >10000 | 33.2 | >10000 | 0.5 |
| 10 | >10000 | 9.3 | >10000 | 3.6 |
| 25 | 222 | 42 | >10000 | 6.8 |
| 1a | >10000 | 11.8 | >10000 | 3.7 |
| 1b | 664 | 108 | >10000 | 23.6 |
| 2a | >10000 | −0.6 | >10000 | 6.1 |
| 2b | 2070 | 82.2 | >10000 | 9.5 |
| 11 | >10000 | 20.1 | >10000 | 4.2 |
| 12 | 6220 | 73.5 | >10000 | 9.2 |
| 13a | >10000 | 39.2 | >10000 | 5.6 |
| 13b | >10000 | 17.3 | >10000 | 2.1 |
| 14 | >10000 | 32.3 | >10000 | 12.2 |
| 15 | >10000 | 10.5 | >10000 | 8.6 |
| 16 | 8480 | 55.2 | >10000 | 5.7 |
| 17 | >10000 | 20.5 | >10000 | 12.5 |
| 18 | 4090 | 79.7 | >10000 | 21.2 |
| 19 | >10000 | 24.6 | >10000 | −4.8 |
| 22 | 8140 | 67.6 | >10000 | 14 |
| 23 | >10000 | 3 | >10000 | 10.7 |
| 20a | >10000 | −13.6 | >10000 | 9.4 |
| 20b | 238 | 104 | 6110 | 67.6 |
| 20c | >10000 | −11.5 | >10000 | 9.0 |
| 20d | 4120 | 91.8 | 5990 | 69.3 |
| 29a | >10000 | −9.1 | >10000 | 5.9 |
| 29b | 88 | 68 | >10000 | 1.3 |
| 54a | >10000 | 15.9 | >10000 | 32 |
| 54b | 87 | 102 | 5250 | 72.4 |
| 51 | 251 | 49.5 | 8910 | 52.3 |
| 52 | >10000 | −0.4 | >10000 | 6.7 |
| 31a | >10000 | 11.3 | >10000 | 10.8 |
| 31b | 563 | 106 | >10000 | 40.0 |
| 32a | >10000 | 11.5 | >10000 | 12.2 |
| 32b | 204 | 93.9 | 9659 | 46.6 |
| 57a | 1450 | 112 | >10000 | 47.5 |
| 57b | >10000 | 12.1 | >10000 | 12.2 |
| 49a | >10000 | 13.6 | >10000 | 18.0 |
| 49b | 422 | 108 | 5450 | 68.9 |
| 58a | >10000 | 12.8 | >10000 | 17.2 |
| 58b | 351 | 98.9 | 5070 | 72.7 |
| 40a | >10000 | 1.2 | >10000 | 15.4 |
| 40b | >10000 | 15.3 | >10000 | 6.6 |
| 53 | 4420 | 74.7 | 6490 | 63.8 |
| 59a | >10000 | 47.5 | >10000 | 21.1 |
| 59b | 75.9 | 94.6 | 6860 | 65.9 |
| 45a | >10000 | 3.0 | >10000 | 10.3 |
| 45b | 590 | 95 | >10000 | 26.7 |
| 37a | 8150 | 68.3 | >10000 | 41.1 |
| 37b | 2040 | 94.5 | 6190 | 64.3 |
| 38a | >10000 | −0.1 | >10000 | 9.0 |
| 38b | 4920 | 100 | 4970 | 73 |
| 69a | >10000 | −4.8 | >10000 | 28.5 |
| 69b | >10000 | 1 | >10000 | 24.5 |
| 44a | >10000 | 27.1 | >10000 | 24.8 |
| 44b | 392 | 101 | 4680 | 85 |
| 61a | >10000 | −0.7 | >10000 | 14.5 |
| 61b | 1940 | 81.6 | >10000 | 19.2 |
| 82 | >10000 | 8.2 | >10000 | 9.4 |
| 47 | >10000 | 12.5 | >10000 | 27.1 |
| 48 | 4670 | 84.3 | 2150 | 90.8 |
| 46a | >10000 | 1.5 | >10000 | 8.4 |
| 46b | 4300 | 81.2 | >10000 | 27.7 |

TABLE 6
Assay Results from Protocol 2

| | | hGIPR % | | hGCGR % |
|---|---|---|---|---|
| | hGIPR | Inhibition | hGCGR | Inhibition |
| Compd# | IC₅₀ (nM) | at 50 μM | IC₅₀ (nM) | at 50 μM |
| 6 | 10.1 | 93 | 6070 | 32 |
| 7 | 2880 | 62 | >50000 | 22 |
| 8 | 107 | 83 | >50000 | 0 |
| 9 | 33.8 | 87 | >50000 | 11 |
| 10 | >50000 | 66 | N/A | |
| 25 | 228 | 62 | 736 | 18 |
| 1a | >50000 | 0 | N/A | |
| 1b | 472 | 94 | >50000 | 52 |
| 2a | 5310 | 5 | N/A | |
| 2b | 388 | 80 | 4400 | 28 |
| 11 | 264 | 89 | >50000 | 0 |
| 12 | 22.3 | 96 | >50000 | 0 |
| 13a | 104 | 81 | 4620 | 30 |
| 13b | >50000 | 65 | N/A | |
| 14 | 80.4 | 91 | >50000 | 66 |
| 15 | >50000 | 66 | N/A | |
| 16 | 64 | 77 | >50000 | 0 |
| 17 | 12300 | 48 | N/A | |
| 18 | 312 | 90 | 5680 | 32 |
| 19 | 298 | 54 | >50000 | 10 |
| 22 | 41.4 | 80 | >50000 | 0 |
| 23 | >50000 | 14 | N/A | |
| 20a | 48500 | 36 | N/A | |
| 20b | 508 | 99 | 33000 | 96 |
| 20c | 5500 | 33 | N/A | |
| 20d | 123 | 100 | 11700 | 79 |
| 29a | >50000 | 36 | N/A | |
| 29b | 744 | 77 | 27000 | 30 |
| 54a | 29000 | 50 | N/A | |
| 54b | 142 | 100 | >50000 | 63 |
| 51 | 91.3 | 116 | >50000 | 96 |
| 52 | >50000 | 102 | N/A | |
| 31a | >50000 | 74 | N/A | |
| 31b | 164 | 102 | 19000 | 61 |
| 32a | >50000 | 62 | N/A | |
| 32b | 105 | 105 | 20900 | 73 |
| 57a | 98 | 110 | 16600 | 88 |
| 57b | >50000 | 72 | N/A | |
| 49a | >50000 | 63 | N/A | |
| 49b | 35.2 | 130 | 12700 | 93 |
| 58a | >50000 | 112 | N/A | |
| 58b | 121 | 116 | 16300 | 94 |
| 40a | >50000 | 0 | N/A | |
| 40b | 306 | 96 | 6600 | 22 |
| 53 | 285 | 97 | 13300 | 98 |
| 59a | >50000 | 83 | N/A | |
| 59b | 26 | 120 | 7690 | 96 |
| 45a | >50000 | 53.5 | N/A | |
| 45b | 199 | 104 | 5070 | 109 |
| 37a | 33100 | 109 | N/A | |
| 37b | 385 | 102 | 25200 | 100 |
| 38a | >50000 | 57.4 | N/A | |
| 38b | 146 | 113 | 7080 | 84 |
| 69a | 16500 | 36 | N/A | |
| 69b | 53.2 | 105 | >50000 | 0 |
| 44a | >50000 | 99 | N/A | |
| 44b | 66.2 | 116 | 19000 | 85 |
| 61a | >50000 | 68 | N/A | |
| 61b | 86 | 93 | >50000 | 52 |
| 82 | 10.6 | 100 | 13500 | 26 |
| 47 | 45100 | 63 | N/A | |
| 48 | 349 | 106 | 19200 | 109 |
| 46a | 18200 | 55 | N/A | |
| 46b | 108 | 105 | 24300 | 74 |
| 83 | 252 | 101 | >50000 | 0 |
| 72a | >50000 | 58 | N/A | |
| 72b | 132 | 103 | 11000 | 99 |
| 73a | >50000 | 101 | N/A | |
| 73b | 52 | 102 | 6760 | 100 |
| 66a | >50000 | 68 | N/A | |
| 66b | 148 | 101 | 12900 | 94 |
| 70a | >50000 | 22 | N/A | |
| 70b | 208 | 91 | 8650 | 101 |
| 67a | >50000 | 0 | N/A | |
| 67b | 192 | 88 | 29200 | 66 |
| 77a | 40900 | 95 | N/A | |
| 77b | 19.3 | 113 | >50000 | 61 |
| 64a | 7450 | 42 | N/A | |
| 64b | 246 | 86 | 13400 | 44 |
| 65a | 33400 | 54 | N/A | |
| 65b | 133 | 107 | 15200 | 86 |
| 68a | 311 | 102 | 5480 | 114 |
| 68b | 31400 | 77 | N/A | |
| 80 | 38.8 | 87 | >50000 | 29 |
| 71a | 7760 | 109 | 43000 | 83 |
| 71b | 53.7 | 120 | 6120 | 100 |
| 109a | 20700 | 54 | 2680 | 22 |
| 109b | 137 | 111 | 11100 | 53 |
| 118 | 41.1 | 100 | 11000 | 25 |
| 119 | 24.8 | 100 | >50000 | 41 |
| 110a | >50000 | 63 | >50000 | 0 |
| 110b | 308 | 110 | 13800 | 85 |
| 85 | 10.8 | 103 | 34300 | 67 |
| 115a | 11000 | 75 | >50000 | 0 |
| 115b | 424 | 89 | >50000 | 0 |
| 116a | 618 | 84 | 5120 | 28 |
| 116b | 69 | 85 | >50000 | 0 |
| 116c | 21800 | 72 | >50000 | 60 |
| 116d | 15100 | 56 | >50000 | 39 |
| 84a | 1040 | 64 | >50000 | 0 |
| 84b | 6.8 | 89 | 4770 | 44 |
| 121a | >50000 | 80 | 50000 | 101 |
| 121b | 39.1 | 101 | 5030 | 103 |
| 122a | 12800 | 89 | 21900 | 81 |
| 122b | 41.5 | 99 | 7570 | 106 |
| 124a | >50000 | 30 | >50000 | 0 |
| 124b | 175 | 95 | N/A | |
| 89a | >50000 | 74 | 5750 | 42 |
| 89b | 53 | 107 | 7900 | 100 |
| 90a | >50000 | 45 | 3190 | 30 |
| 90b | 473 | 77 | 9550 | 37 |
| 91a | 34800 | 70 | 18900 | 34 |
| 91b | 222 | 93 | 14100 | 38 |
| 96a | 19600 | 92 | >50000 | 71 |
| 96b | 37.9 | 99 | 5810 | 100 |
| 86a | 18000 | 63 | 11500 | 46 |
| 86b | 54.8 | 104 | 17500 | 72 |
| 92 | 17800 | 94 | >50000 | 51 |
| 93 | 56 | 115 | 14200 | 69 |
| 94 | 6070 | 89 | 15100 | 29 |
| 95 | 21.9 | 103 | 47000 | 43 |
| 125 | 465 | 87 | >50000 | 51 |
| 126 | 270 | 87 | >50000 | 26 |
| 128 | 99 | 93 | >50000 | 36 |
| 129 | 54 | 104 | 17500 | 66 |
| 130 | 407 | 85 | 26100 | 30 |
| 131 | 337 | 76 | 12500 | 47 |
| 132 | 158 | 79 | 18700 | 49 |
| 98 | 28 | 106 | 13800 | 19 |
| 99 | 81 | 97 | 22300 | 43 |
| 133 | 236 | 90 | >50000 | 31 |
| 97a | >50000 | 82 | >50000 | 52 |
| 97b | 125 | 89 | 21800 | 95 |
| 102 | 32 | 69 | >50000 | 0 |
| 103 | 12 | 84 | >50000 | 5 |
| 104 | 5.7 | 91 | 4590 | 26 |
| 105 | 415 | 87 | 20700 | 88 |
| 106 | 53 | 100 | 12500 | 35 |
| 150 | 141 | 97 | 12600 | 35 |
| 149 | 372 | 91 | 16700 | 92 |
| 147 | 264 | 81 | 26700 | 55 |
| 145 | 114 | 106 | 22400 | 92 |
| 144 | 22 | 106 | 9210 | 96 |
| 143 | 92 | 94 | 22200 | 94 |
| 142 | 5.8 | 83 | 3850 | 46 |
| 141 | 129 | 80 | 8190 | 39 |
| 140 | 7.5 | 48 | 21100 | 73 |
| 139 | 72 | 95 | >50000 | 69 |
| 138 | 260 | 92 | >50000 | 22 |
| 137 | 96 | 93 | >50000 | 62 |
| 156 | 36 | 86 | 28800 | 80 |
| 157 | 69 | 76 | 13600 | 43 |
| 154a | >50000 | 74 | >50000 | 73 |
| 154b | 92 | 101 | 30400 | 100 |
| 152a | 3.9 | 109 | >50000 | 71 |
| 152b | 1700 | 93 | 38800 | 47 |
| 155a | >50000 | 44 | 9010 | 28 |
| 155b | 15 | 84 | 5120 | 39 |
| 158 | 94 | 77 | 13200 | 44 |
| 159a | 16 | 110 | 12200 | 117 |
| 159b | 42100 | 90 | 16200 | 107 |
| 161a | >50000 | 38 | >50000 | 47 |
| 161c | >50000 | 0 | 3690 | 30 |
| 161d | 145 | 91 | 18800 | 47 |
| 161b | 201 | 85 | >50000 | 61 |
| 162a | 943 | 79 | 14000 | 41 |
| 162b | 4.5 | 91 | 4230 | 54 |
| 163a | 4510 | 47 | N/A | |
| 163b | 125 | 103 | 16500 | 48 |
| 166a | 9890 | 57 | 1370 | 22 |
| 166b | 117 | 87 | 32300 | 73 |
| 167a | 333 | 91 | >50000 | 93 |
| 167b | 3690 | 29 | >50000 | 0 |
| 165a | 7.2 | 98 | 3580 | 50 |
| 165b | >50000 | 0 | N/A | |
| 169a | 84.2 | 85 | >50000 | 40 |
| 169b | 590 | 63 | >50000 | 17 |
| 170a | >50000 | 38 | >50000 | 0 |
| 170b | 20 | 104 | 8750 | 87 |
| 174a | 99 | 77 | 11400 | 109 |
| 174b | 107 | 55 | N/A | |
| 164a | 6.8 | 90 | N/A | |
| 164b | 38300 | 59 | N/A | |
| 168a | 1250 | 89 | 10400 | 114 |
| 168b | 15 | 92 | 9510 | 88 |
| 197a | >21300 | 73 | 12500 | 36 |
| 197b | 4.2 | 116 | 11700 | 99 |
| 190a | 370 | 84 | >50000 | 25 |
| 190b | 18900 | 32 | >50000 | 20 |
| 187a | 3950 | 85 | >50000 | 0 |
| 187b | 40 | 91 | 27500 | 28 |
| 189a | 1230 | 83 | >50000 | 0 |
| 189b | 362 | 87 | >50000 | 16 |
| 196a | 16800 | 40 | >50000 | 0 |
| 196b | 322 | 88 | >50000 | 0 |
| 188a | >50000 | 66 | 2800 | 26 |
| 188b | 275 | 89 | >50000 | 25 |
| 193 | 281 | 104 | 4100 | 31 |
| 194 | 147 | 77 | >50000 | 0 |
| 195 | 40 | 88 | >23300 | 74 |
| 191 | 197 | 93 | 5120 | 36 |
| 192a | 42 | 105 | 50000 | 102 |
| 192b | 70 | 110 | 50000 | 105 |
| 200 | 103 | 87 | 12000 | 29 |
| 205a | 4230 | 104 | 50000 | 104 |
| 205b | 46 | 117 | 50000 | 113 |
| 201 | 333 | 94 | 3440 | 56 |
| 202 | 73 | 73 | 950 | 49 |
| 203 | 301 | 98 | 1850 | 38 |
| 208 | 68 | 114 | 23800 | 105 |
| 212 | 33 | 94 | 7190 | 35 |
| 215a | >50000 | 91.1 | N/A | |
| 215b | 32 | 111 | 16400 | 118 |
| 210 | 52 | 38 | >50000 | 28 |
| 209 | 21 | 93 | 2780 | 52 |
| 204 | 5.3 | 90 | 8540 | 58 |
| 211 | 26 | 105 | 18300 | 95 |
| 216a | >50000 | 24 | 13300 | 56 |
| 216b | 187 | 84 | 23000 | 52 |
| 206a | >50000 | 64 | 7160 | 38 |
| 206b | 83 | 91 | >50000 | 31 |
| 217a | 45 | 90 | 9040 | 29 |
| 217b | 5470 | 66 | 17900 | 48 |
| 207a | 184 | 88 | >50000 | 47 |
| 207b | 15600 | 45 | 5240 | 42 |
| 198 | >50000 | 68 | >50000 | 50 |
| 236a | 251 | 88 | >50000 | 0 |
| 236b | 6.2 | 100 | >50000 | 48 |
| 235a | 92 | 88 | 14000 | 67 |
| 235b | 3240 | 67 | 39300 | 72 |
| 238 | 15 | 89 | >50000 | 54 |
| 239 | 9.8 | 92 | 22100 | 82 |
| 223a | 10100 | 69 | >50000 | 0 |
| 223b | 21 | 89 | 2060 | 35 |
| 224a | 8600 | 71 | >50000 | 37 |
| 224b | 20.9 | 92 | >50000 | 44 |
| 225a | 35500 | 74 | 17100 | 30 |
| 225b | 15 | 105 | >50000 | 39 |
| 226a | 171 | 71 | >50000 | 0 |
| 226b | 26 | 97 | >50000 | 37 |
| 230a | 6340 | 87 | >50000 | 58 |
| 230b | 9.8 | 113 | >50000 | 74 |
| 240a | 6810 | 77 | 6070 | 37 |
| 240b | 10 | 90 | >50000 | 44 |
| 285 | 249 | 92 | >50000 | 36 |
| 284 | 426 | 83 | 6150 | 32 |
| 282 | 273 | 80 | 3830 | 35 |
| 281 | 76 | 82 | >50000 | 34 |
| 280 | 257 | 111 | >50000 | 60 |
| 279 | 70 | 103 | 9780 | 55 |
| 237a | >50000 | 42 | >50000 | 0 |
| 237b | 129 | 87 | >50000 | 6 |
| 227a | 12220 | 63 | >50000 | 49 |
| 227b | 5.0 | 104 | >50000 | 0 |
| 221a | 21900 | 51 | >50000 | 0 |
| 221b | 5.3 | 98 | >50000 | 32 |
| 229a | 36400 | 35 | >50000 | 25 |
| 229b | 12 | 90 | >50000 | 56 |
| 231a | 19400 | 37 | >50000 | 0 |
| 231b | 40 | 83 | >50000 | 0 |
| 233a | 39000 | 34 | 20700 | 16 |
| 233b | 37 | 82 | >50000 | 16 |
| 241a | >50000 | 0 | >50000 | 20 |
| 241b | 36 | 68 | 75700 | 16 |
| 278 | 179 | 94 | >50000 | 7 |
| 277 | 227 | 90 | >50000 | 9 |
| 275 | 455 | 75 | 5670 | 25 |
| 274 | 408 | 97 | >50000 | 0 |
| 273 | 43 | 31 | >50000 | 23 |
| 272 | 352 | 91 | >50000 | 0 |
| 271 | 43 | 87 | >50000 | 18 |
| 270 | 216 | 15 | >50000 | 0 |
| 269 | 94 | 86 | >50000 | 0 |
| 267 | 100 | 87 | >50000 | 0 |
| 266 | 449 | 94 | >50000 | 0 |
| 220a | 9910 | 69 | >50000 | 0 |
| 220b | 50 | 83 | >50000 | 0 |
| 222a | 43000 | 24 | >50000 | 43 |
| 222b | 26 | 96 | >50000 | 67 |
| 228a | 49500 | 57 | >50000 | 26 |
| 228b | 6.4 | 99 | >50000 | 0 |
| 232a | 1450 | 59 | >50000 | 0 |
| 232b | 41 | 81 | >50000 | 32 |
| 234a | 21700 | 53 | >50000 | 38 |
| 234b | 44 | 88 | >50000 | 44 |
| 218 | 117 | 114 | >50000 | 73 |
| 242a | 5020 | 85 | >50000 | 34 |
| 242b | 3.7 | 99 | >50000 | 0 |
| 265 | 100 | 86 | >50000 | 35 |
| 264 | 116 | 87 | >50000 | 0 |
| 263 | 19 | 96 | >50000 | 40 |
| 262 | 27 | 89 | N/A | |
| 260 | 63 | 95 | N/A | |
| 259 | 33 | 100 | N/A | |
| 258 | 373 | 70 | >50000 | 18 |
| 256 | 220 | 81 | 8620 | 30 |
| 255 | 394 | 67 | >50000 | 0 |
| 254 | 53 | 101 | >50000 | 0 |
| 252 | 98 | 85 | >50000 | 36 |
| 251 | 431 | 89 | >50000 | 0 |
| 250 | 68 | 91 | >50000 | 45 |
| 246 | 84 | 77 | >50000 | 0 |
| 311b | 18 | 100 | >50000 | 23 |
| 312a | 16000 | 97 | >50000 | 44 |
| 312b | 8.4 | 102 | >50000 | 63 |
| 316a | 5.4 | 101 | 20000 | 37 |
| 316b | 3150 | 91 | 26600 | 53 |
| 245 | 466 | 97 | >50000 | 0 |
| 244 | 92 | 84 | >50000 | 0 |
| 310a | 9600 | 36 | >50000 | 16 |
| 310b | 35 | 89 | >50000 | 16 |
| 302a | 46 | 95 | >50000 | 0 |
| 302b | 1630 | 92 | >50000 | 60 |
| 303a | 19 | 95 | >50000 | 68 |
| 303b | 4790 | 64 | >50000 | 19 |
| 305a | 15900 | 79 | >50000 | 21 |
| 305b | 58 | 90 | >50000 | 20 |
| 306a | 1780 | 81 | 1100 | 27 |
| 306b | 32 | 89 | >50000 | 20 |
| 307a | 24600 | 77 | >50000 | 0 |
| 307b | 66 | 95 | 10000 | 34 |
| 309a | 1480 | 89 | >50000 | 58 |
| 309b | 4.1 | 90 | >50000 | 33 |
| 308a | 1410 | 70 | >50000 | 0 |
| 308b | 14 | 99 | >50000 | 0 |
| 286 | 0.9 | 105 | 35600 | 49 |
| 304a | 3700 | 83 | >50000 | 0 |
| 304b | 32 | 90 | >50000 | 0 |
| 288a | 9950 | 60 | >50000 | 0 |
| 288b | 100 | 88 | >50000 | 0 |
| 289a | 11900 | 49 | >50000 | 0 |
| 289b | 7.1 | 94 | >50000 | 0 |
| 287a | 5630 | 71 | >50000 | 0 |
| 287b | 11 | 87 | >50000 | 0 |
| 290a | 11100 | 73 | >50000 | 23 |
| 290b | 6.0 | 96 | 17500 | 49 |
| 291a | 5880 | 84 | >50000 | 0 |
| 291b | 10 | 98 | >50000 | 0 |
| 293a | 1040 | 100 | >50000 | 52 |
| 293b | 12 | 106 | 46600 | 45 |
| 294a | >50000 | 88 | >50000 | 32 |
| 294b | 4.5 | 105 | >50000 | 36 |
| 295a | 11600 | 90 | >50000 | 70 |
| 295b | 11 | 107 | >50000 | 70 |
| 296a | >50000 | 93 | >50000 | 38 |
| 296b | 11 | 104 | 33000 | 39 |
| 292a | >50000 | 98 | >50000 | 49 |
| 292b | 11 | 110 | 50000 | 102 |
| 297a | 9310 | 94 | >50000 | 24 |
| 297b | 4.1 | 104 | >50000 | 52 |
| 314a | >50000 | 95 | >50000 | 0 |
| 314b | 50000 | 121 | >50000 | 17 |
| 314c | 27 | 118 | >50000 | 51 |
| 314d | 141 | 120 | >50000 | 0 |
| 313a | 1040 | 109 | >50000 | 0 |
| 313b | 35 | 111 | >50000 | 0 |
| 315 | 7.9 | 99 | >50000 | 41 |
| 299a | 10300 | 64 | >50000 | 0 |
| 299b | 2.7 | 100 | >50000 | 60 |
| 298a | 2.0 | 98 | 50000 | 111 |
| 298b | 106 | 96 | 50000 | 106 |
| 317a | 783 | 75 | >50000 | 0 |
| 317b | 10 | 89 | >50000 | 0 |
| 318a | 2930 | 84 | >50000 | 0 |
| 318b | 2.4 | 110 | >50000 | 23 |
| 319 | 2.7 | 99 | 6530 | 53 |
| 320 | 10 | 76 | 2990 | 64 |
| 321a | >50000 | 0 | >50000 | 0 |
| 321b | 56 | 82 | >50000 | 0 |

### In Vivo Body Weight Test

Forty 6-7-week-old male B6-hGIPR (hgipr)KI/KI (Strain: T054143, C57BL/6JGpt-Giprem2Cin(hGIPR)/Gpt) mice (approximately 24-25 weeks old, approximately 51-52 g) were housed 4 per cage in a 12 h light: 12 hr dark cycle (6 am-6 pm) at standard temperature (22° C.) with ad libitum access to 60% high fat diet (Research diets: D12492) and water. Mice were placed on HFD at weaning and led in for 18 weeks prior to study start. Body weight and food intake were measured using a Sartorius BP 6100 Precision Balance scale. Mice were acclimated by PO sham dosing with 100 μL of water for three days prior to study start. Baseline body weight was used to randomize mice into 5 dosing groups.

From days 0-21, body weights were measured daily and food intake was measured daily using the hopper method. Food was replenished on (Mondays and Thursdays). Mice were co-administered with an oral dose QD with vehicle 1 (0.2% Tween80:98% (0.5% w/v Methylcellulose in deionized water) (v/v)) and Compound 85 at 10 mg/kg or 20 mg/kg and a subcutaneous dose QD with vehicle 2 (1.35% Propylene Glycol/98.65% Dulbecco's 0.01 M PBS pH=7.4 (v/v)) or 0.05 mg/kg liraglutide.

On Day 21, to measure exposure of Compound 85 at all doses and liraglutide after chronic dosing, approximately 20 μL blood samples were collected 1, 2, 4, 8, 14 and 24 hours post dosing. A subset of mice (n=4) were euthanized post blood collection and tissues (brain, hypothalamus, liver and epidydimal fat pad) were harvested at 2 h and 14h. Blood samples were placed into dipotassium ethylenediaminetetraacetic acid (EDTA) tubes and centrifuged at 10,000×g for 10 minutes. Plasma samples were aliquoted to 1.7 mL Eppendorf tubes, and stored in −20° C.

Pharmacokinetics: Blood was collected from all animals on study into EDTA tubes at each timepoint (1 and 24 HPD) and kept on wet ice until centrifuging at 10,000×g for 10 minutes at 4° C. Resulting plasma was collected and kept at −80° C. until analysis.

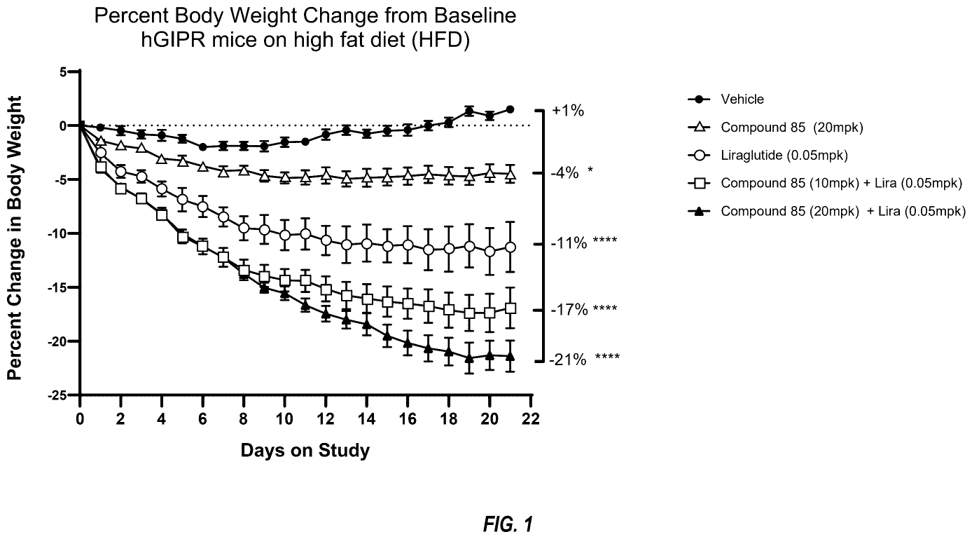

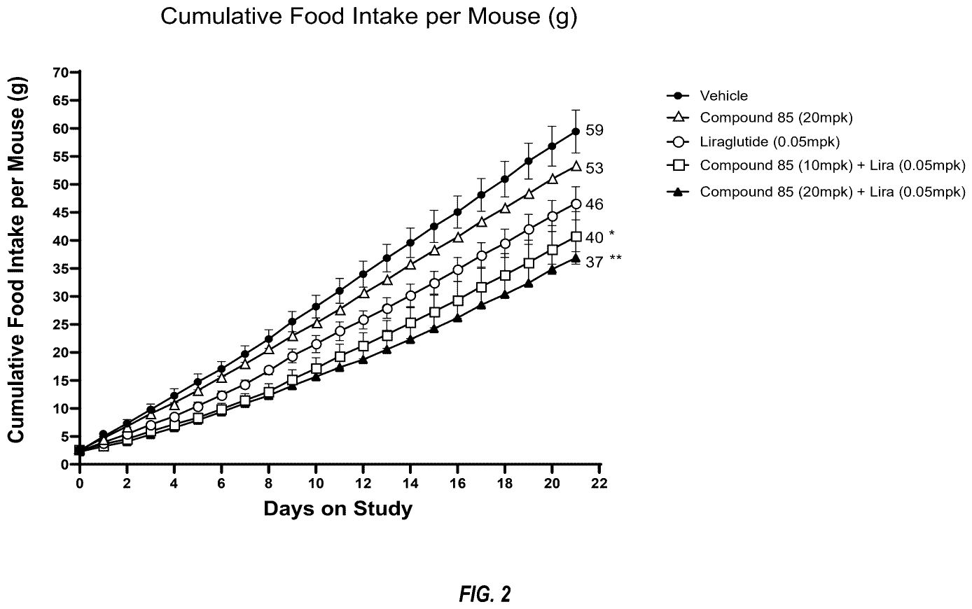

FIG. 1 and FIG. 2 show weight loss in mice on a high fact diet treated with Compound 85 as a monotherapy and in combination with Lira ((0.05 mpk). Mice showed less cumulative food intake and greater weight loss using the combination therapy compared with either the inventive compound or Lira alone.

All publications, including but not limited to, issued patents, patent applications, and journal articles, cited in this application are each herein incorporated by reference in their entirety.

Although the invention has been described above with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

1. A compound of Formula (I) or a pharmaceutically acceptable salt thereof: 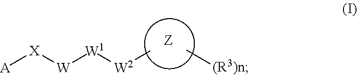
wherein:
A is absent, —(C₀-C₄)alkyl-C(O)OR², OR², or 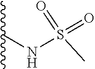 [SMILES: *NS(C)(=O)=O]
wherein the —N(H)S(O)₂—CH₃ is optionally substituted with one to three halogens;
R² is H or (C₁-C₆)alkyl;
X is —W³-L-W⁴—, wherein L is absent, —C(O)—N(H)—, or —N(H)—C(O)—, wherein W³ is bonded to A and W⁴ is bonded to W;
W³ is absent, phenyl, phenyl fused to a 5 to 6 membered heteroaryl, a 5 to 6 membered heteroaryl, or a 5 to 6 membered heteroaryl fused to a 5 to 6 membered heteroaryl, said phenyl fused to a 5 to 6 membered heteroaryl, 5 to 6 membered heteroaryl fused to a 5 to 6 membered heteroaryl, and a 5 to 6 membered heteroaryl having 1 to 3 heteroatoms selected independently from nitrogen, sulfur and oxygen;
wherein W³ is optionally mono-, di-, or tri-substituted independently with hydroxy, halo, oxo, (C₁-C₄)alkyl or (C₁-C₄)alkoxy;
W⁴ is absent, phenyl, 5 to 6 membered heteroaryl, phenyl fused to a (C₃-C₇)cycloalkyl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl, or a phenyl fused to a 5 to 7 membered heteroaryl; wherein said 5 to 6 membered heteroaryl, phenyl fused to a 5 to 7 membered heterocycloalkyl, 5 to 6 membered heteroaryl fused to a (C₃-C₇)cycloalkyl, 5 to 6 membered heteroaryl fused to a 5 to 7 membered heterocycloalkyl, 5 to 6 membered heteroaryl fused to a 5 to 6 membered heteroaryl and phenyl fused to a 5 to 7 membered heteroaryl have 1 to 4 heteroatoms independently selected from nitrogen, sulfur and oxygen;
wherein when W is absent and W¹ is a ring system, W⁴ optionally shares one atom with W¹ in a spiro linkage;
wherein W⁴ is optionally mono-, di-, or tri-substituted independently with halo, oxo, (C₁-C₄)alkyl or (C₁-C₄)alkoxy;
W is absent, —*C(O)—N(H)—, —*N(H)—C(O)—, or —*N(H)S(O)₂—, wherein the “*” represents the point of attachment of W to X;
W¹ is —CH(Y)—, —C(R¹⁰)(R¹¹)—, (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl;
wherein adjacent ring atoms of each of said (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl are bonded to W and W²;
wherein each of said 4 to 7 membered heterocyclic, 4 to 8 membered bridged heterocyclic, or 4 to 7 membered heterocycloalkenyl have 1 to 3 heteroatoms selected independently from nitrogen, oxygen and sulfur;
wherein R¹⁰ and R¹¹ are each independently H or (C₁-C₄)alkyl; or R¹⁰ and R¹¹ are taken together with the carbon to which they are attached to form a 3 to 6 membered cycloalkyl ring; or R¹⁰ and R¹¹ can be taken together with the carbon to which they are attached to form a (C₅-C₆)cycloalkyl ring fused to a phenyl ring;
wherein each of said (C₃-C₇)cycloalkyl, (C₅-C₆)cycloalkyl ring fused to a phenyl ring, 4 to 7 membered heterocyclic, 4 to 8 membered bridged heterocyclic, or 4 to 7 membered heterocycloalkenyl is optionally mono- or di-substituted independently with (C₁-C₄)alkyl, halo, (C₁-C₄)alkoxy, or trifluoromethyl, or is optionally mono-substituted with phenyl, or are spiro linked to a cyclopropyl or oxetanyl;
wherein Y is (C₁-C₆)alkyl, phenyl or (C₃-C₇)cycloalkyl) each optionally mono-substituted with phenyl or trifluoromethyl;
W² is —*C(O)—N(H)—, —*C(O)—N(H)—(C₁-C₃)alkyl-, —*NH—C(O)NH—, —*O—C(O)—NH—, —*C(O)—O—, —*S(O)₂—, —*S(O)₂—N(H)—, —*S(O)₂—C(H)₂—, or —*N(R⁴)—C(O)NH—, wherein the (C₁-C₃)alkyl is optionally substituted with (C₁-C₃)alkyl, halo, (C₁-C₃)alkoxy or (C₁-C₃)alkoxy(C₁-C₃)alkyl and the “*” represents the point of attachment to W¹;
Z is 5 to 7 membered heterocycloalkyl, phenyl, 5 to 6 membered heteroaryl, C₃-C₆ cycloalkyl, 5-10 membered bridged cycloalkyl, phenyl fused to a 5 to 6 membered heteroaryl, a 5 to 6 membered heteroaryl fused to a 5 to 7 membered cycloalkyl, a 5 to 6 membered heteroaryl fused to a 5 to 7 membered heterocycloalkyl, phenyl fused to a 5 to 7 membered heterocycloalkyl or phenyl fused to a 5 to 7 membered cycloalkyl, wherein said 5 to 7 membered heterocycloalkyl, 5 to 6 membered heteroaryl, phenyl fused to a 5 to 6 membered heteroaryl, 5 to 6 membered heteroaryl fused to a 5 to 7 membered cycloalkyl, 5 to 6 membered heteroaryl fused to a 5 to 7 membered heterocycloalkyl and phenyl fused to a 5 to 7 membered heterocycloalkyl have 1 to 3 heteroatoms selected independently from nitrogen, oxygen and sulfur;
each R³ is independently (C₁-C₆)alkyl, (C₁-C₆)alkoxy, halo, phenyl, mono-, di- or tri-halo(C₁-C₄)alkyl, —SCF₃, —OCF₃, —OCHF₂, (C₃-C₇)cycloalkyl, said (C₃-C₇)cycloalkyl optionally substituted independently with 1 or 2 (C₁-C₄)alkyl, or mono-, di- or tri-halo(C₁-C₄)alkyl;
R⁴ is (C₁-C₄)alkyl or phenyl; and
n is 0, 1, 2, or 3;
with the proviso that W⁴—W—W¹—W²—Z is not

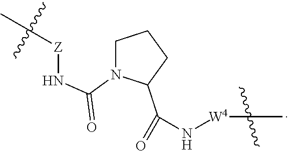

2. The compound of claim 1, wherein A is absent or —(C₀-C₄)alkyl-C(O)OR²; W³ is phenyl; W⁴ is phenyl; and wherein W³ and W⁴ are each independently optionally mono-, di-, or tri-substituted independently with halo, (C₁-C₄)alkyl or (C₁-C₄)alkoxy.

3. The compound of claim 1, wherein A is absent or —(C₀-C₄)alkyl-C(O)OR²; W³ is a 5 to 6 membered heteroaryl; and W⁴ is phenyl; and wherein W³ and W⁴ are each independently optionally mono-, di-, or tri-substituted independently with halo, (C₁-C₄)alkyl or (C₁-C₄)alkoxy.

4. The compound of claim 1, wherein A is absent or —(C₀-C₄)alkyl-C(O)OR²; W³ is absent; and W⁴ is a 5 to 6 membered heteroaryl fused to a 5 to 6 membered heteroaryl having 1 to 4 heteroatoms selected independently from nitrogen, sulfur and oxygen; and wherein W⁴ is optionally mono-, di-, or tri-substituted independently with halo, oxo, (C₁-C₄)alkyl or (C₁-C₄)alkoxy.

5. The compound of claim 1, wherein A is absent or —(C₀-C₄)alkyl-C(O)OR²; W³ is phenyl; W⁴ is phenyl fused to a 5 to 7 membered heteroaryl having 1 to 3 heteroatoms selected independently from nitrogen, sulfur and oxygen; and wherein W³ and W⁴ are each independently optionally mono-, di-, or tri-substituted independently with halo, oxo, (C₁-C₄)alkyl or (C₁-C₄)alkoxy.

6. The compound of claim 1, wherein A is absent or —(C₀-C₄)alkyl-C(O)OR²; W³ is phenyl; and W⁴ is a 5 to 6 membered heteroaryl having 1 to 4 heteroatoms selected independently from nitrogen, sulfur and oxygen; and wherein W⁴ is optionally mono-, di-, or tri-substituted independently with halo, oxo, (C₁-C₄)alkyl or (C₁-C₄)alkoxy.

7. The compound of claim 2, wherein Z is phenyl.

8. The compound of claim 2, wherein Z is pyridinyl.

9. The compound of claim 2, wherein Z—(R³)ₙ is

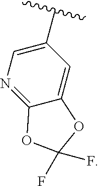 [SMILES: *c1cnc2c(c1)OC(F)(F)O2]

10. The compound of claim 2, wherein Z is

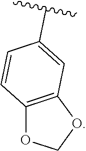 [SMILES: *c1ccc2c(c1)OCO2]

11. The compound of claim 2, wherein W is —N(H)—C(O)—, wherein the X—W—W¹ is X—N(H)—C(O)—W¹.

12. The compound of claim 2, wherein W is —N(H)S(O)₂—, wherein the X—W—W¹ is X—N(H)—S(O)₂—W¹.

13. The compound of claim 2, wherein W is absent.

14. The compound of claim 2, wherein R³ is (C₁-C₃)alkyl and n is 1.

15. The compound of claim 2, wherein W² is —*C(O)—N(H)—.

16. The compound of claim 2, wherein W¹ is pyrrolidinyl.

17. The compound of claim 2, wherein W¹ is —C(R¹⁰)(R¹¹)— and R¹⁰ and R¹¹ are taken together with the carbon to which they are attached to form a 3 to 6 membered cycloalkyl ring.

18. The compound of claim 2, wherein W¹ is —C(R¹⁰)(R¹¹)— and R¹⁰ is (C₁-C₄)alkyl and R¹¹ is H.

19. The compound of claim 2, wherein W¹ is cyclopentyl.

20. The compound of claim 2, wherein W¹ is cyclohexyl.

21. The compound of claim 2, wherein W¹ is cyclohexenyl.

22. The compound of claim 2, wherein W¹ is azetidinyl.

23. The compound of claim 2, wherein W¹ is piperidinyl.

24. The compound of claim 2, wherein W¹ is a 4 to 8 membered bridged heterocyclic.

25. The compound of claim 2, wherein W¹ is azabicyclo[2.1.1]hexanyl.

26. The compound of claim 2, wherein W—W¹—W² is 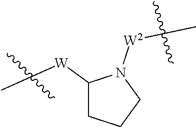
and W is absent.

27. The compound of claim 2, wherein W—W¹—W² is

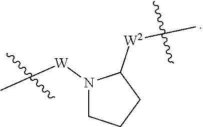

28. The compound of claim 2, wherein W¹ is imidazolinyl.

29. The compound of claim 2, wherein W¹ is pyrazolinyl.

30. The compound of claim 2, wherein W³ is mono substituted with fluoro.

31. The compound of claim 1, wherein A is absent or —(C₀-C₄)alkyl-C(O)OR²; W³ is phenyl; W is —N(H)—C(O)—, wherein the X—W—W¹ is X—N(H)—C(O)—W¹; W² is —*C(O)—N(H)—; R³ is (C₁-C₃)alkyl; n is 1; and W³ is optionally mono-substituted with fluoro or methyl.

32. The compound of claim 31, wherein Z is phenyl.

33. The compound of claim 31, wherein Z is pyridinyl.

34. The compound of claim 31, wherein Z—(R³)ₙ is

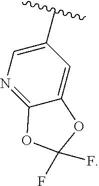 [SMILES: *c1cnc2c(c1)OC(F)(F)O2]

35. The compound of claim 31, wherein Z is

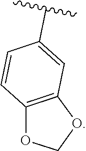 [SMILES: *c1ccc2c(c1)OCO2]

36. The compound of claim 31, wherein W¹ is cyclopentyl.

37. The compound of claim 31, wherein W¹ is cyclohexyl.

38. The compound of claim 31, wherein W¹ is cyclohexenyl.

39. The compound of claim 31, wherein W¹ is azetidinyl.

40. The compound of claim 31, wherein W¹ is piperidinyl.

41. The compound of claim 31, wherein W¹ is a 4 to 8 membered bridged heterocyclic.

42. The compound of claim 31, wherein W¹ is azabicyclo[2.1.1]hexanyl.

43. The compound of claim 31, wherein W—W¹—W² is

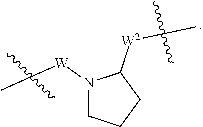

44. The compound of claim 1, wherein W¹ is chosen from imidazolinyl, pyrazolinyl, cyclopentyl, cyclohexyl, cyclohexenyl, azetidinyl, piperidinyl, and azabicyclo[2.1.1]hexanyl.

45.-52. (canceled)

53. The compound of claim 1, wherein W—W¹—W² is 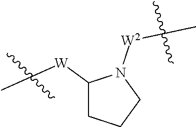
and W is absent.

54. The compound of claim 1, wherein W—W¹—W² is 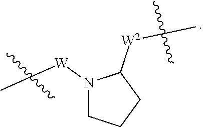
and W is —N(H)—C(O)—, wherein the X—W—W¹ is X—N(H)—C(O)—W¹.

55. The compound of claim 1, wherein W² is —*NH—C(O)NH—, —*O—C(O)—NH—, —*C(O)—O—, —*S(O)₂—, —*S(O)₂—N(H)— or —*S(O)₂—C(H)₂— or —*N(R⁴)—C(O)NH—, wherein the “*” represents the point of attachment to W¹.

56.-62. (canceled)

63. The compound of claim 55, wherein W is —N(H)—C(O)—, wherein the X—W—W¹ is X—N(H)—C(O)—W¹.

64. The compound of claim 1, wherein W¹ is (C₃-C₇)cycloalkyl, 4 to 7 membered heterocyclic, 4 to 8 membered bridged heterocyclic, or 4 to 7 membered heterocycloalkenyl having the formula 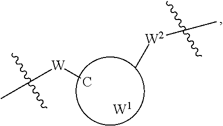
wherein W is bonded to a W¹ ring carbon and said W¹ ring carbon is a stereocenter in an R configuration.

65. The compound of claim 1, wherein W¹ is (C₃-C₇)cycloalkyl, 4 to 7 membered heterocyclic, 4 to 8 membered bridged heterocyclic, or 4 to 7 membered heterocycloalkenyl having the formula 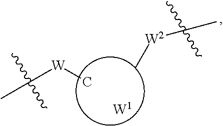
wherein W is bonded to a W¹ ring carbon and said W¹ ring carbon is a stereocenter in an S configuration.

66. The compound of claim 1, wherein W¹ is (C₃-C₇)cycloalkyl, 4 to 7 membered heterocyclic, 4 to 8 membered bridged heterocyclic, or 4 to 7 membered heterocycloalkenyl having the formula 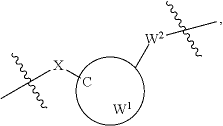
wherein W is absent, X is bonded to a W¹ ring carbon and said W¹ ring carbon is a stereocenter in an R configuration.

67. The compound of claim 1, wherein W¹ is (C₃-C₇)cycloalkyl, 4 to 7 membered heterocyclic, 4 to 8 membered bridged heterocyclic, or 4 to 7 membered heterocycloalkenyl having the formula 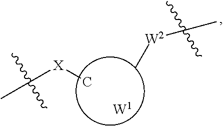
wherein W is absent, X is bonded to a W¹ ring carbon and said W¹ ring carbon is a stereocenter in an S configuration.

68. The compound of claim 1, wherein W¹ is (C₃-C₇)cycloalkyl, 4 to 7 membered heterocyclic, 4 to 8 membered bridged heterocyclic, or 4 to 7 membered heterocycloalkenyl having the formula 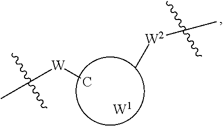
wherein W² is bonded to a W¹ ring carbon and said W¹ ring carbon is a stereocenter in an R configuration.

69. The compound of claim 1, wherein W¹ is (C₃-C₇)cycloalkyl, 4 to 7 membered heterocyclic, 4 to 8 membered bridged heterocyclic, or 4 to 7 membered heterocycloalkenyl having the formula 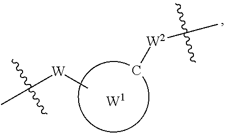
wherein W² is bonded to a W¹ ring carbon and said W¹ ring carbon is a stereocenter in an S configuration.

70. The compound of claim 1, wherein W¹ is (C₃-C₇)cycloalkyl, 4 to 7 membered heterocyclic, 4 to 8 membered bridged heterocyclic, or 4 to 7 membered heterocycloalkenyl having the formula 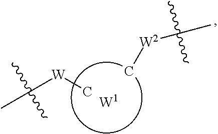
wherein W is bonded to a W¹ ring carbon and said W¹ ring carbon is a stereocenter in an R configuration; and wherein W² is bonded to a W¹ ring carbon and said W¹ ring carbon is a stereocenter in an R configuration.

71. The compound of claim 1, wherein W¹ is (C₃-C₇)cycloalkyl, 4 to 7 membered heterocyclic, 4 to 8 membered bridged heterocyclic, or 4 to 7 membered heterocycloalkenyl having the formula 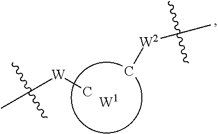
wherein W is bonded to a W¹ ring carbon and said W¹ ring carbon is a stereocenter in an S configuration; and wherein W² is bonded to a W¹ ring carbon and said W¹ ring carbon is a stereocenter in an S configuration.

72. The compound of claim 1, wherein W¹ is (C₃-C₇)cycloalkyl, 4 to 7 membered heterocyclic, 4 to 8 membered bridged heterocyclic, or 4 to 7 membered heterocycloalkenyl having the formula 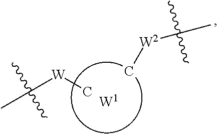
wherein W is absent, X is bonded to a W¹ ring carbon and said W¹ ring carbon is a stereocenter in an R configuration; and wherein W² is bonded to a W¹ ring carbon and said W¹ ring carbon is a stereocenter in an R configuration.

73. The compound of claim 1, wherein W¹ is (C₃-C₇)cycloalkyl, 4 to 7 membered heterocyclic, 4 to 8 membered bridged heterocyclic, or 4 to 7 membered heterocycloalkenyl having the formula 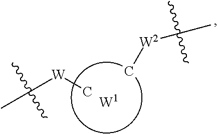
wherein W is absent, X is bonded to a W¹ ring carbon and said W¹ ring carbon is a stereocenter in an S configuration; and wherein W² is bonded to a W¹ ring carbon and said W¹ ring carbon is a stereocenter in an S configuration.

74.-75. (canceled)

76. The compound of claim 26, wherein W—W¹—W² is 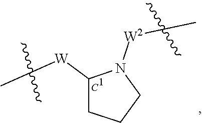
W is absent, and the C¹ carbon is a stereocenter in an R configuration.

77. The compound of claim 26, wherein W—W¹—W² is 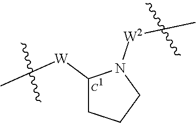
and the C¹ carbon is a stereocenter in an S configuration.

78.-79. (canceled)

80. A pharmaceutical composition comprising a therapeutically effective amount of a compound and/or a pharmaceutically acceptable salt of claim 1 and a pharmaceutically acceptable excipient.

81. A pharmaceutical combination composition comprising: a therapeutically effective amount of a composition comprising:
a first compound, said first compound being a compound of claim 1 or a pharmaceutically acceptable salt of said compound;
a second compound, said second compound being a GLP-1 inhibitor, a GCGR agonist or a GIPR agonist; and
a pharmaceutically acceptable excipient.

82. A pharmaceutical combination composition comprising: a therapeutically effective amount of a composition comprising:
a first compound, said first compound being a compound of claim 1 or a pharmaceutically acceptable salt of said compound;
a second compound, said second compound being a GLP-1 inhibitor; and
a third compound, said third compound being a GCGR agonist or a GIPR agonist; and
a pharmaceutically acceptable excipient.

83. A kit comprising:
a. a first compound, said first compound being a compound of claim 1, a prodrug thereof, or a pharmaceutically acceptable salt of said compound or of said prodrug and a pharmaceutically acceptable excipient in a first unit dosage form;
b. a second compound, said second compound being a GLP-1 inhibitor, a GCGR agonist or a GIPR agonist and a pharmaceutically acceptable excipient in a second unit dosage form; and
c. means for containing said first and second dosage forms wherein the amounts of first and second compounds result in a therapeutic effect.

84. (canceled)

85. A method of modulating GIPR activity, the method comprising administering an effective amount of a compound of claim 1.

86. A method of treating, preventing or ameliorating a GIPR-mediated disease or condition in a subject in need thereof comprising administering to the subject an effective amount of a compound of claim 1.

87. The method of claim 86, wherein the disease is chosen from Type II diabetes, elevated glucose levels, elevated insulin levels, dyslipidemia, metabolic syndrome (Syndrome X or insulin resistance syndrome), glucosuria, metabolic acidosis, Type 1 diabetes, obesity, conditions exacerbated by obesity, pancreatitis, cirrhosis and Turner's & Cushing's syndrome.

88. A method of treating a disease or condition selected from Type 2 diabetes, elevated glucose levels, elevated insulin levels, dyslipidemia, metabolic syndrome (Syndrome X or insulin resistance syndrome), glucosuria, metabolic acidosis, Type 1 diabetes, obesity, conditions exacerbated by obesity, pancreatitis, cirrhosis and Turner's & Cushing's syndrome, comprising administering to the subject an effective amount of a compound of claim 1.

89. The method of claim 88, wherein the condition is obesity.

90. The method of claim 88, wherein the condition is type II diabetes.

91. A method of treating a disease or condition chosen from Type 2 diabetes, elevated glucose levels, elevated insulin levels, dyslipidemia, metabolic syndrome (Syndrome X or insulin resistance syndrome), glucosuria, metabolic acidosis, Type 1 diabetes, obesity, conditions exacerbated by obesity, pancreatitis, cirrhosis and Turner's & Cushing's syndrome, comprising administering to the subject an effective amount of a compound of claim 1 or a pharmaceutical composition thereof, and
a second compound, said second compound being a GLP-1 inhibitor, a GCGR agonist or a GIPR agonist.

92. The method of claim 91, wherein the second compound is a GLP-1 inhibitor.

93. The method of claim 91, wherein the condition is obesity.

94. A compound selected from (R)-4′-(1-((2,2-difluorobenzo[d][1,3]dioxol-5-yl)carbamoyl)-3,3-dimethylazetidine-2-carboxamido)-[1,1′-biphenyl]-4-carboxylic acid, and pharmaceutically acceptable salts thereof.

95. A compound selected from (R)-2-(5-(1H-[1,2,3]triazolo[4,5-b]pyridin-5-yl)-3-fluoropyridin-2-yl)-N-(2,2-difluoro-[1,3]dioxolo[4,5-b]pyridin-6-yl)pyrrolidine-1-carboxamide, and pharmaceutically acceptable salts thereof.

96. A compound selected from (R)-4′-(3,3-dimethyl-1-((4-(trifluoromethyl)phenyl)carbamoyl)azetidine-2-carboxamido)-[1,1′-biphenyl]-4-carboxylic acid, and pharmaceutically acceptable salts thereof.

97. A compound selected from (R)-2-(5-(1H-[1,2,3]triazolo[4,5-b]pyridin-5-yl)-3-fluoropyridin-2-yl)-N-(2,2-difluorobenzo[d][1,3]dioxol-5-yl)pyrrolidine-1-carboxamide, and pharmaceutically acceptable salts thereof.

98. A compound selected from (R)—N²-(4-(1H-[1,2,3]triazolo[4,5-b]pyridin-5-yl)phenyl)-N¹-(4-isopropylphenyl)-3,3-dimethylazetidine-1,2-dicarboxamide, and pharmaceutically acceptable salts thereof.

99. A compound selected from (R)-4′-(1-((4-isopropylphenyl)carbamoyl)-3,3-dimethylazetidine-2-carboxamido)-[1,1′-biphenyl]-4-carboxylic acid, and pharmaceutically acceptable salts thereof.

100. A compound selected from 4-(6-((2S,3S)-1-((2,2-difluorobenzo[d][1,3]dioxol-5-yl)carbamoyl)-3-methylpyrrolidin-2-yl)-5-fluoropyridin-3-yl)benzoic acid, and pharmaceutically acceptable salts thereof.

101. A compound selected from (S)-5-(4-(1-((4-isopropylphenyl)carbamoyl)-3,3-dimethylazetidine-2-carboxamido)phenyl)-6-methylpicolinic acid, and pharmaceutically acceptable salts thereof.

102. A compound selected from (R)-4-(6-(1-((2,2-difluorobenzo[d][1,3]dioxol-5-yl)carbamoyl)pyrrolidin-2-yl)-5-fluoropyridin-3-yl)benzoic acid, and pharmaceutically acceptable salts thereof.

103. A compound selected from 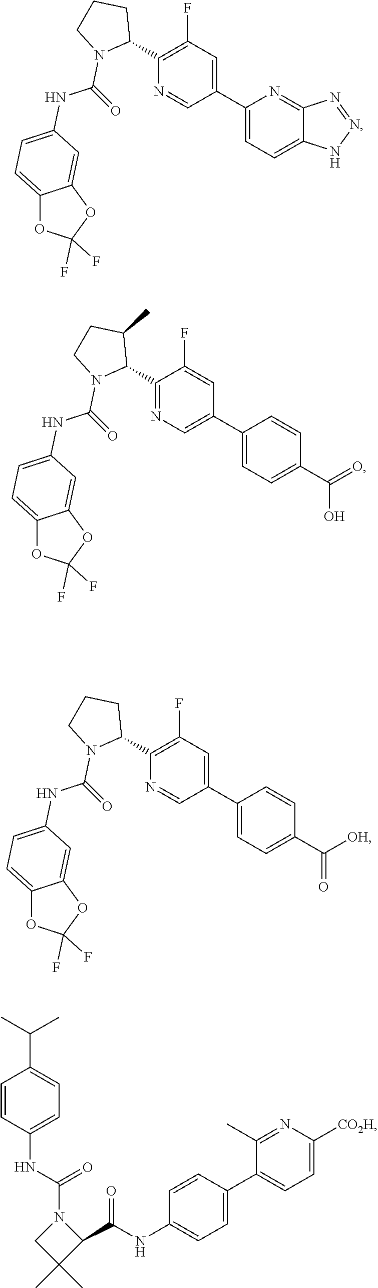 [SMILES: O=C(Nc1ccc2c(c1)OC(F)(F)O2)N1CCC[C@@H]1c1ncc(-c2ccc3[nH]nnc3n2)cc1F ; C[C@@H]1CCN(C(=O)Nc2ccc3c(c2)OC(F)(F)O3)[C@H]1c1ncc(-c2ccc(C(=O)O)cc2)cc1F ; O=C(O)c1ccc(-c2cnc([C@H]3CCCN3C(=O)Nc3ccc4c(c3)OC(F)(F)O4)c(F)c2)cc1 ; Cc1nc(C(=O)O)ccc1-c1ccc(NC(=O)[C@@H]2N(C(=O)Nc3ccc(C(C)C)cc3)CC2(C)C)cc1] 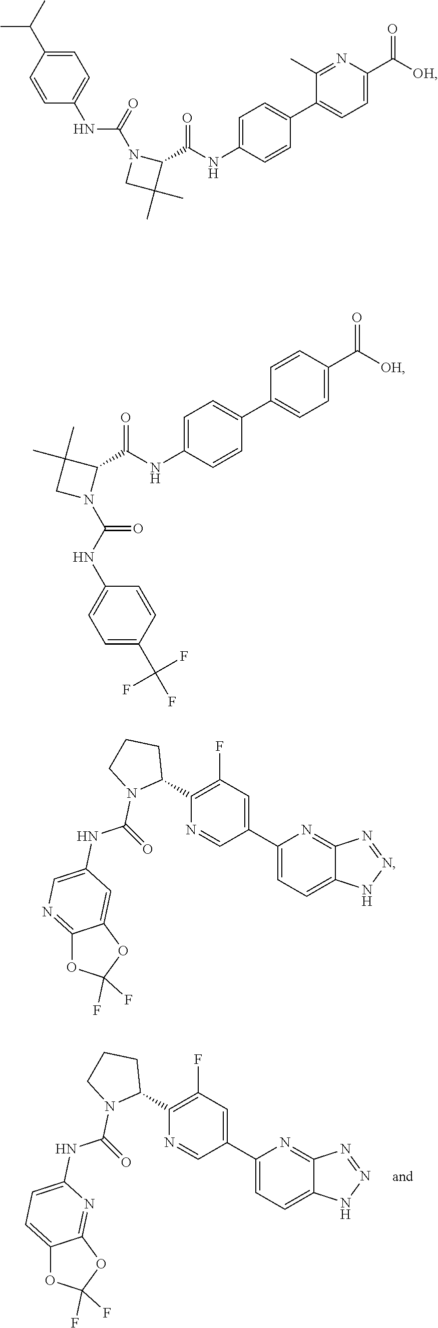 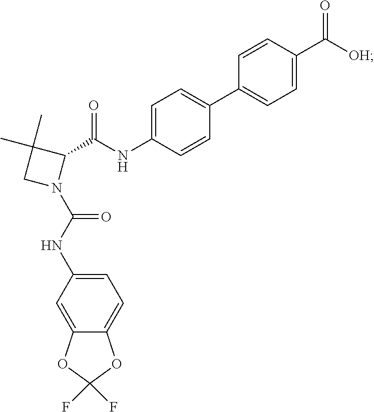 [SMILES: CC1(C)CN(C(=O)Nc2ccc3c(c2)OC(F)(F)O3)[C@H]1C(=O)Nc1ccc(-c2ccc(C(=O)O)cc2)cc1]
or a pharmaceutically acceptable salt thereof.

104. A compound having the structure 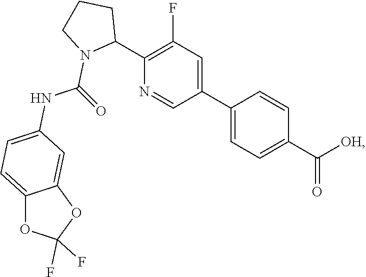 [SMILES: O=C(O)c1ccc(-c2cnc(C3CCCN3C(=O)Nc3ccc4c(c3)OC(F)(F)O4)c(F)c2)cc1]
or a pharmaceutically acceptable salt thereof.

105. A compound having the structure 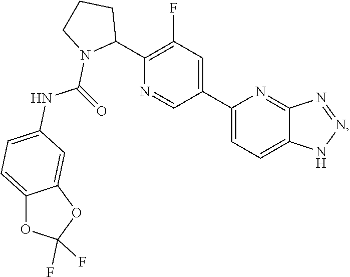 [SMILES: O=C(Nc1ccc2c(c1)OC(F)(F)O2)N1CCCC1c1ncc(-c2ccc3[nH]nnc3n2)cc1F]
or a pharmaceutically acceptable salt thereof.

106. The compound of claim 1, wherein
A is (C₀-C₄)alkyl-C(O)OR²;
R² is H or (C₁-C₆)alkyl;
W³ is phenyl optionally mono- or di-substituted independently with halo, (C₁-C₄)alkyl or (C₁-C₄)alkoxy;
W⁴ is pyridinyl optionally mono- or di-substituted independently with halo, (C₁-C₄)alkyl or (C₁-C₄)alkoxy;
W—W¹—W² is 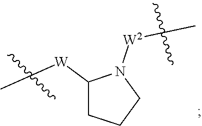
and W is absent;
W² is —*C(O)NH—, wherein the “*” represents the point of attachment to W¹;
Z is phenyl or pyridinyl;
R³ is (C₁-C₆)alkyl, halo, mono-, di- or tri-halo(C₁-C₄)alkyl or (C₃-C₇)cycloalkyl; and
n is 1 or 2.

107. The compound of claim 106, wherein W—W¹—W² is 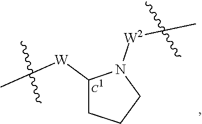
and the C¹ carbon is a stereocenter in an R configuration.

108. The compound of claim 106, wherein
W³ is phenyl optionally mono-substituted independently with halo or (C₁-C₄)alkyl;
W⁴ is pyridinyl optionally mono-substituted independently with halo or (C₁-C₄)alkyl;
Z is phenyl or pyridinyl;
R³ is (C₁-C₆)alkyl, halo or mono-, di- or tri-halo(C₁-C₄)alkyl; and
n is 1 or 2.

109. The compound of claim 1, wherein
A is (C₀-C₄)alkyl-C(O)OR²;
R² is H or (C₁-C₆)alkyl;
W³ is phenyl optionally mono- or di-substituted independently with halo, (C₁-C₄)alkyl or (C₁-C₄)alkoxy;
W⁴ is pyridinyl optionally mono- or di-substituted independently with halo, (C₁-C₄)alkyl or (C₁-C₄)alkoxy;
W—W¹—W² is 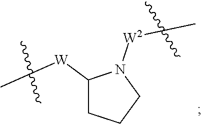
and W is absent;
W² is —*C(O)NH—, wherein the “*” represents the point of attachment to W¹;
Z is phenyl fused to a 5 to 7 membered heterocycloalkyl;
R³ is (C₁-C₆)alkyl, halo, mono-, di- or tri-halo(C₁-C₄)alkyl or (C₃-C₇)cycloalkyl; and
n is 1 or 2.

110. The compound of claim 109, wherein W² is bonded to the Z phenyl.

111. The compound of claim 1, wherein
A is (C₀-C₄)alkyl-C(O)OR²;
R² is H or (C₁-C₆)alkyl;
W³ is pyridyl optionally mono- or di-substituted independently with halo, (C₁-C₄)alkyl or (C₁-C₄)alkoxy;
W⁴ is phenyl optionally mono- or di-substituted independently with halo, (C₁-C₄)alkyl or (C₁-C₄)alkoxy;
W—W¹—W² is 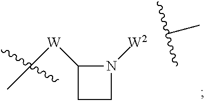
and W is absent;
W² is —*C(O)NH—, wherein the “*” represents the point of attachment to W¹;
Z is phenyl or pyridinyl;
R³ is (C₁-C₆)alkyl, halo, mono-, di- or tri-halo(C₁-C₄)alkyl or (C₃-C₇)cycloalkyl; and
n is 1 or 2.

112. The compound of claim 1, wherein
A is —C(O)OR², or absent;
R² is H;
W³ is phenyl, pyridinyl or 1H-[1,2,3]triazolo[4,5-b]pyridin-5-yl optionally mono- or di-substituted independently with (C₁-C₃)alkyl or halo;
W⁴ is phenyl or pyridinyl optionally mono- or di-substituted independently with (C₁-C₄)alkyl or halo;
W—W¹—W² is 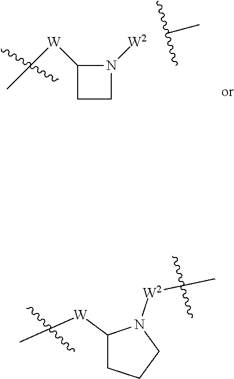
and wherein W¹ is optionally mono- or di-substituted with methyl; and W is absent;
W² is —*C(O)NH—, wherein the “*” represents the point of attachment to W¹;
Z is phenyl, pyridinyl or 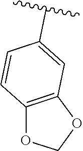 [SMILES: *c1ccc2c(c1)OCO2]
or;
R³ is (C₁-C₄)alkyl or halo; and
n is 1 or 2.

113. The compound of claim 112, wherein
A is —C(O)OR²;
R² is H;
W³ is phenyl or pyridinyl optionally mono- or di-substituted independently with (C₁-C₃)alkyl, fluoro or chloro;
W⁴ is phenyl or pyridinyl optionally mono- or di-substituted independently with fluoro or chloro;
W—W¹—W² is 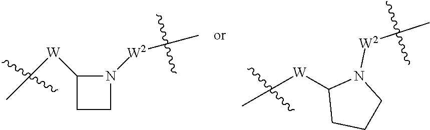
and wherein W¹ is optionally mono- or di-substituted with methyl; and W is absent;
W² is —*C(O)NH—, wherein the “*” represents the point of attachment to W¹;
Z is phenyl, 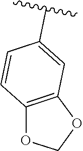 [SMILES: *c1ccc2c(c1)OCO2]
or;
R³ is (C₁-C₄)alkyl, fluoro or chloro; and
n is 1 or 2.

114. The compound of claim 112, wherein
A is absent;
W³ is 1H-[1,2,3]triazolo[4,5-b]pyridin-5-yl;
W⁴ is phenyl or pyridinyl optionally mono- or di-substituted independently with fluoro or chloro;
W—W¹—W² is 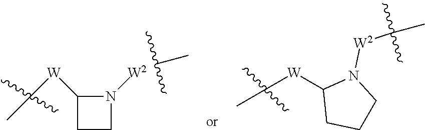
and wherein W¹ is optionally mono- or di-substituted with methyl; and W is absent;
W² is —*C(O)NH—, wherein the “*” represents the point of attachment to W¹;
Z is phenyl, 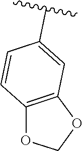 [SMILES: *c1ccc2c(c1)OCO2]
or;
R³ is (C₁-C₄)alkyl, fluoro or chloro; and
n is 1 or 2.

115. The compound of claim 1, wherein
A is —C(O)OR², or absent;
R² is H;
W³ is phenyl, pyridinyl or 1H-[1,2,3]triazolo[4,5-b]pyridin-5-yl optionally mono- or di-substituted independently with (C₁-C₃)alkyl or halo;
W⁴ is phenyl or pyridinyl optionally mono- or di-substituted independently with (C₁-C₄)alkyl or halo;
W—W¹—W² is 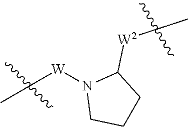
or;
W is absent or —*NHC(O)—, wherein the “*” represents the point of attachment to X;
W¹ is optionally mono- or di-substituted with methyl;
W² is —*C(O)NH—, wherein the “*” represents the point of attachment to W¹;
Z is phenyl, pyridinyl, 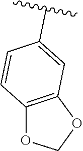 [SMILES: *c1ccc2c(c1)OCO2]
or;
R³ is (C₁-C₄)alkyl or halo; and
n is 1 or 2.

116. The compound of claim 115, wherein
A is C(O)OR²;
R² is H;
W³ is phenyl or pyridinyl optionally mono- or di-substituted independently with (C₁-C₃)alkyl, fluoro or chloro;
W⁴ is phenyl or pyridinyl optionally mono- or di-substituted independently with fluoro or chloro;
W—W¹—W² is 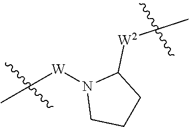
or;
W is absent;
W¹ is optionally mono- or di-substituted with methyl;
W² is —*C(O)NH—, wherein the “*” represents the point of attachment to W¹;
Z is phenyl, 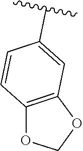 [SMILES: *c1ccc2c(c1)OCO2]
or;
R³ is (C₁-C₄)alkyl, fluoro or chloro; and
n is 1 or 2.

117. The compound of claim 115, wherein
A is C(O)OR²;
R² is H;
W³ is phenyl or pyridinyl optionally mono- or di-substituted independently with (C₁-C₃)alkyl, fluoro or chloro;
W⁴ is phenyl or pyridinyl optionally mono- or di-substituted independently with fluoro or chloro;
W—W¹—W² is 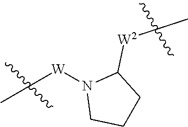
or;
W is —*NHC(O)—, wherein the “*” represents the point of attachment to X;
W¹ is optionally mono- or di-substituted with methyl;
W² is —*C(O)NH—, wherein the “*” represents the point of attachment to W¹;
Z is phenyl, 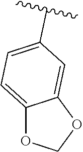 [SMILES: *c1ccc2c(c1)OCO2]
or;
R³ is (C₁-C₄)alkyl, fluoro or chloro; and
n is 1 or 2.

118. The compound of claim 115, wherein
A is absent;
W³ is 1H-[1,2,3]triazolo[4,5-b]pyridin-5-yl;
W⁴ is phenyl or pyridinyl optionally mono- or di-substituted independently with fluoro or chloro;
W—W¹—W² is 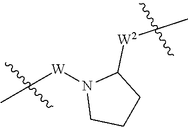
or;
W is absent;
W¹ is optionally mono- or di-substituted with methyl;
W² is —*C(O)NH—, wherein the “*” represents the point of attachment to W¹;
Z is phenyl, 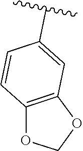 [SMILES: *c1ccc2c(c1)OCO2]
or;
R³ is (C₁-C₄)alkyl, fluoro or chloro; and
n is 1 or 2.

119. The compound of claim 115, wherein
A is absent;
W³ is 1H-[1,2,3]triazolo[4,5-b]pyridin-5-yl;
W⁴ is phenyl or pyridinyl optionally mono- or di-substituted independently with fluoro or chloro;
W—W¹—W² is 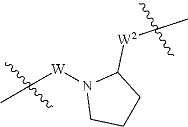
or;
W is —*NHC(O)—, wherein the “*” represents the point of attachment to X;
W¹ is optionally mono- or di-substituted with methyl;
W² is —*C(O)NH—, wherein the “*” represents the point of attachment to W¹;
Z is phenyl, 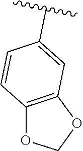 [SMILES: *c1ccc2c(c1)OCO2]
or;
R³ is (C₁-C₄)alkyl, fluoro or chloro; and
n is 1 or 2.

120. A compound having the structure, 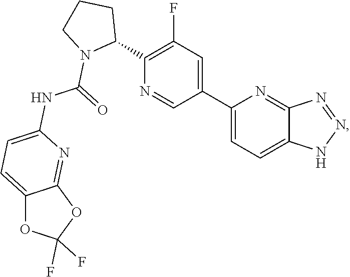 [SMILES: O=C(Nc1ccc2c(n1)OC(F)(F)O2)N1CCC[C@@H]1c1ncc(-c2ccc3[nH]nnc3n2)cc1F]
or a pharmaceutically acceptable salt thereof.

121. A compound selected from (R)-2-(5-(1H-[1,2,3]triazolo[4,5-b]pyridin-5-yl)-3-fluoropyridin-2-yl)-N-(2,2-difluoro-[1,3]dioxolo[4,5-b]pyridin-5-yl)pyrrolidine-1-carboxamide, and pharmaceutically acceptable salts thereof.
